(19) United States
(12) Patent Application Publication
(10) Pub. No.: US 2025/0032623 A1
(43) Pub. Date: Jan. 30, 2025

(54) HETEROBIFUNCTIONAL COMPOUNDS AND THEIR USE IN TREATING DISEASE

(71) Applicant: HALDA THERAPEUTICS OPCO, INC., New Haven, CT (US)

(72) Inventors: Samuel W. GERRITZ, Guilford, CT (US); Katherine J. KAYSER-BRICKER, Branford, CT (US); Kyle J. EASTMAN, Killingworth, CT (US); Taavi NEKLESA, Orange, CT (US); Kanak Shail RAINA, Branford, CT (US)

(21) Appl. No.: 18/696,722

(22) PCT Filed: Oct. 4, 2022
(86) PCT No.: PCT/US2022/045606

§ 371 Date: Mar. 28, 2024

Related U.S. Application Data
(60) Provisional application No. 63/251,716, filed on Oct. 4, 2021.

(51) Int. Cl. A61K 47/55; A61K 47/54; A61K 47/60; A61P 35/00

(52) U.S. Cl. CPC A61K 47/55; A61K 47/545; A61K 47/60; A61P 35/00

(57) ABSTRACT

The invention provides heterobifunctional compounds comprising an effector protein binding moiety selected from mTor, PLK1, CDK1, CDK2, CDK9, BRD4, AURKA, AURKB, MEK, Src, c-KIT, KIF11, HSP90, tubulin, proteasome, topoisomerase or HD AC which is linked to a moiety that binds to a target protein selected from BTK, androgen receptor or IDH1. Pharmaceutical compositions and their use in treating disease, such as cancer, are also disclosed.

### CROSS-REFERENCE TO RELATED APPLICATION

This application claims priority to U.S. provisional patent application No. 63/251,716, filed on Oct. 4, 2021, the contents of which are hereby incorporated by reference in their entirety.

### FIELD OF THE INVENTION

The invention provides heterobifunctional compounds, pharmaceutical compositions, and their use in treating disease, such as cancer.

### BACKGROUND

Cancer continues to be a significant health problem despite the substantial research efforts and scientific advances reported in the literature for treating this disease. Solid tumors, including prostate cancer, breast cancer, and lung cancer remain highly prevalent among the world population. The incidence of prostate cancer increases with age, and with increasing longevity of human subjects, there continues to be a corresponding rise in the number of patients suffering from prostate cancer. Breast cancer is one of the most common cancers among women and is a leading cause of death for women between ages 50-55. Lung cancer is a leading cause of death among cancer patients, where over 85% of lung cancers are non-small cell lung cancer (NSCLC). Many lung cancers are attributed to tobacco smoking. Current treatment options for these cancers are not effective for all patients and/or can have substantial adverse side effects.

New therapies are needed to address this unmet need in cancer therapy. In particular, new therapies are needed that achieve an anti-cancer effect through a different mechanism than commonly available therapies. Exemplary mechanisms for common anti-cancer therapies include (a) alkylation of DNA which limits ability of the cell to reproduce, (b) topoisomerase inhibition, in which the therapeutic agent inhibits the activity of a topoisomerases thereby limiting separation of strands of DNA, and (c) mitotic inhibition, where the therapeutic agent reduces ability of the cell to divide. New therapies that achieve an anti-cancer effect through a different mechanism present an opportunity to treat cancers more effectively and/or to treat cancers that have become resistant to currently available medicines.

The present invention addresses the foregoing needs and provides other related advantages.

### SUMMARY

The invention provides heterobifunctional compounds, pharmaceutical compositions, and their use in treating disease, such as cancer. In particular, one aspect of the invention provides a collection of heterobifunctional compounds, such as a compound represented by Formula I:

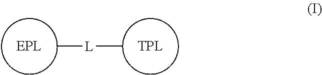

or a pharmaceutically acceptable salt thereof, where the variables are as defined in the detailed description. Further description of additional collections of heterobifunctional compounds are described in the detailed description. The compounds may be part of a pharmaceutical composition comprising a pharmaceutically acceptable carrier.

Another aspect of the invention provides a method of treating cancer. The method comprises administering to a patient in need thereof a therapeutically effective amount of a compound described herein, such as a compound of Formula I to treat the cancer.

Another aspect of the invention provides a method of treating hepatitis. The method comprises administering to a patient in need thereof a therapeutically effective amount of a compound described herein, such as a compound of Formula I, to treat the hepatitis.

Another aspect of the invention provides a method of causing death of a cancer cell. The method comprises contacting a cancer cell with an effective amount of a compound described herein, such as a compound of Formula I, to cause death of the cancer cell.

### DETAILED DESCRIPTION

The invention provides heterobifunctional compounds, pharmaceutical compositions, and their use in treating disease, such as cancer. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

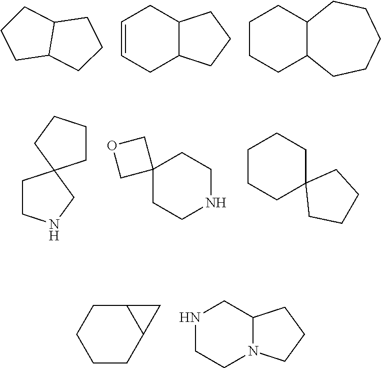

Exemplary bridged bicyclics include:

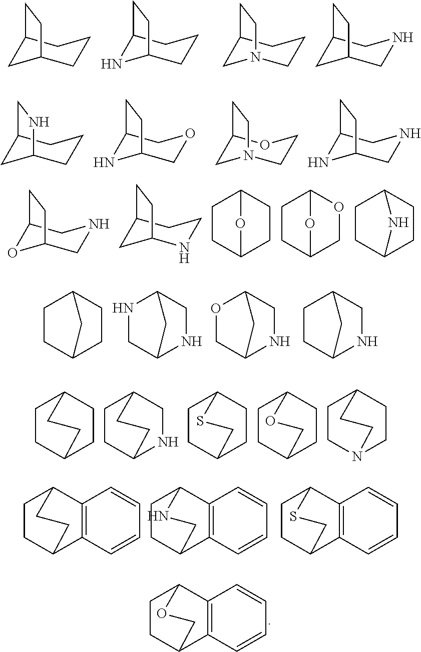

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)-group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “haloaryl” refers to an aryl group that is substituted with at least one halogen. Exemplary haloaryl groups include chlorophenyl (e.g., 3-chlorophenyl, 4-chlorophenyl), fluorophenyl, and the like. The term “phenylene” refers to a bivalent phenyl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 Tc electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted. The term “haloheteroaryl” refers to a heteroaryl group that is substituted with at least one halogen. Exemplary haloheteroaryl groups include chloropyridine, fluoropyridine, chloropyrazole, fluoropyrazole, and the like. The term “heteroarylene” refers to a bivalent heteroaryl group. Similarly, the terms “pyrazolylene”, “imidazolylene”, and “pyrrolylene”, respectively refer to bivalent pyrazolyl, imidazolyl, and pyrrolyl groups. Similarly, the terms “pyridinylene” and “pyrimidinylene”, respectively refer to bivalent pyridinyl and pyrimidinyl groups.

As used herein, the terms “heterocycle,” “heterocyclyl,” “heterocyclic radical,” and “heterocyclic ring” are used interchangeably and refer to a stable 5-to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or *NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “heterocyclylene” refers to a bivalent heterocyclyl group.

As used herein, the term “heterocycloakyl” refers to a saturated heterocyclyl. The term “heterocycloakyl” refers to a bivalent heterocycloakyl group.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}2; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}2; —P(O)₂R^{∘}; —P(O)R^{∘}2; —OP(O)R^{∘}2; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{●}, -(haloR^{●}), —(CH₂)₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}3, —OSiR^{●}3, —C(O)SR^{●}, —(C₁₋₄ straight or branched alkylene)C(O)OR^{●}, or —SSR^{●}.

Each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(Rt)S(O)₂Rt; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋aliphatic, R^{†} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}2, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich:Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N+(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “chloroalkyl” refers to an alkyl group that is substituted with at least one chloro. The term “bromoalkyl” refers to an alkyl group that is substituted with at least one bromo. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The term “heteroalkyl” refers to an alkyl group in which one or more carbon atoms has been replaced by a heteroatom (e.g., N, O, or S). Exemplary heteroalkyl groups include —OCH₃, —CH₂OCH₃, —CH₂CH₂N(CH₃)₂, and —CH₂CH₂OH. The heteroalkyl group may contain, for example, from 2-4, 2-6, or 2-8 atoms selected from the group consisting of carbon and a heteroatom (e.g., N, O, or S). The phrase 3-8 membered heteroalkyl refers to a heteroalkyl group having from 3 to 8 atoms selected from the group consisting of carbon and a heteroatom. The term “heteroalkylene” refers to a bivalent heteroalkyl group.

The terms “alkenyl” and “alkynyl” are art-recognized and refer to unsaturated aliphatic groups analogous in length and possible substitution to the alkyls described above, but that contain at least one double or triple bond respectively. The term “haloalkenyl” refers to an alkenyl group that is substituted with at least one halogen. The term “fluoroalkenyl” refers to an alkenyl group that is substituted with at least one fluoro. The term “nitroalkenyl” refers to an alkenyl group that is substituted with at least one nitro.

The term “carbocyclylene” refers to a bivalent cycloaliphatic group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “═O” substituent. For example, a cyclopentane susbsituted with an oxo group is cyclopentanone.

The term “amino” is art-recognized and refers to both unsubstituted and substituted amines, e.g., a moiety that may be represented by the general formulas:

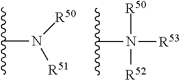

wherein R⁵⁰, R⁵¹, R⁵² and R⁵³ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a 3-7 membered cycloalkyl, a 4-7 membered cycloalkenyl, 5-10 membered heteroaryl, or 3-10 membered heterocyclyl; and m is zero or an integer in the range of 1 to 8.

The term “amido” is art-recognized and refers to both unsubstituted and substituted amides, e.g., a moiety that may be represented by the general formulas:

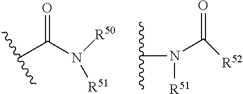

wherein R⁵⁰ and R⁵¹ each independently represent a hydrogen, an alkyl, an alkenyl, —(CH₂)ₘ-R⁶¹, or R⁵⁰ and R⁵¹, taken together with the N atom to which they are attached complete a heterocycle having from 4 to 8 atoms in the ring structure; R⁶¹ represents an aryl, a 3-7 membered cycloalkyl, a 4-7 membered cycloalkenyl, 5-10 membered heteroaryl, or 3-10 membered heterocyclyl; and m is zero or an integer in the range of 1 to 8; and R⁵² is an alkyl, an alkenyl, or —(CH₂)ₘ-R⁶¹.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Heterobifunctional Compounds

The invention provides heterobifunctional compounds. The compounds are generally represented by the following formula:

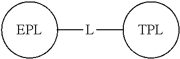

or a pharmaceutically acceptable salt thereof, wherein EPL is a moiety that binds to an effector protein selected from mTOR, PLK1, CDK1, CDK2, CDK9, BRD4, AURKA, AURKB, MEK, Src, c-KIT, KIF11, HSP90, tubulin, proteasome, topoisomerase, or HDAC; L is a linker; and TPL is a moiety that binds to a target protein selected from KRAS, HER2, or EGFR.

The compounds may be used in the pharmaceutical compositions and therapeutic methods described herein. Exemplary compounds are described in the following sections, along with exemplary procedures for making the compounds.

### Part A: Compounds of Formula I

Another aspect of the invention provides a compound represented by Formula I:

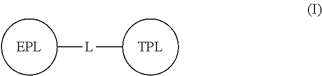

or a pharmaceutically acceptable salt thereof; wherein:
EPL is a moiety that binds to an effector protein selected from mTOR, PLK1, CDK1, CDK2, CDK9, BRD4, AURKA, AURKB, MEK, Src, c-KIT, KIF11, HSP90, tubulin, proteasome, topoisomerase, or HDAC;
L is a linker; and
TPL is a moiety that binds to a target protein selected from KRAS, HER2, or EGFR.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

The compound may be further characterized according to, for example, the identity of L and/or TPL. Exemplary further embodiments for L and TPL are provided in Part C below.

As generally defined above, EPL is a moiety that binds to an effector protein selected from mTOR, PLK1, CDK1, CDK2, CDK9, BRD4, AURKA, AURKB, MEK, Src, c-KIT, KIF11, HSP90, Tubulin, Proteasome, Topoisomerase, or HDAC. In certain embodiments, the EPL is a moiety that binds to mTOR, PLK1, CDK1, CDK2, CDK9, or BRD4. In certain embodiments, the EPL is a moiety that binds to mTOR. In certain embodiments, the EPL is a moiety that binds to PLK1. In certain embodiments, the EPL is a moiety that binds to CDK1. In certain embodiments, the EPL is a moiety that binds to CDK2. In certain embodiments, the EPL is a moiety that binds to CDK9. In certain embodiments, the EPL is a moiety that binds to BRD4. In certain embodiments, the EPL is a moiety that binds to AURKA. In certain embodiments, the EPL is a moiety that binds to AURKB. In certain embodiments, the EPL is a moiety that binds to MEK. In certain embodiments, the EPL is a moiety that binds to Src. In certain embodiments, the EPL is a moiety that binds to c-KIT. In certain embodiments, the EPL is a moiety that binds to KIF11. In certain embodiments, the EPL is a moiety that binds to HSP90. In certain embodiments, the EPL is a moiety that binds to tubulin. In certain embodiments, the EPL is a moiety that binds to proteasome. In certain embodiments, the EPL is a moiety that binds to topoisomerase. In certain embodiments, the EPL is a moiety that binds to HDAC.

A. Moiety for mTOR

In certain embodiments, the EPL is a moiety that binds to Mammalian Target of Rapamycin (mTOR). Exemplary moieties that bind mTOR are reported in the literature, including:

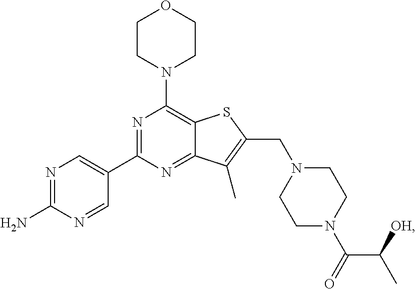 [SMILES: Cc1c(CN2CCN(C(=O)[C@H](C)O)CC2)sc2c(N3CCOCC3)nc(-c3cnc(N)nc3)nc12]

as described in Derynck, M. K. et al., WO 2012/164060;

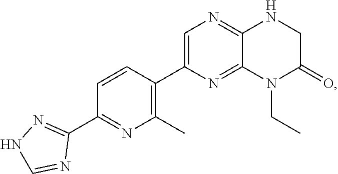 [SMILES: CCN1C(=O)CNc2ncc(-c3ccc(-c4nc[nH]n4)nc3C)nc21]

as described in Xu, S. et al., WO 2013/138557, Raymon, H. et al., WO 2014/172424 and WO 2014/172425;

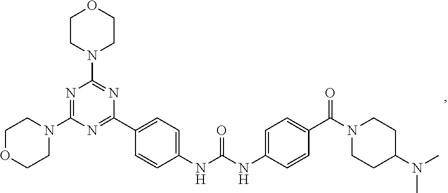 [SMILES: CN(C)C1CCN(C(=O)c2ccc(NC(=O)Nc3ccc(-c4nc(N5CCOCC5)nc(N5CCOCC5)n4)cc3)cc2)CC1]

as described in Venkatesan, A. M. et al., US2017/224696;

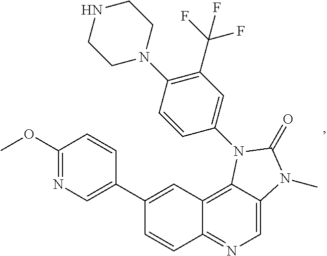

as described in Wu, F. et al., WO 2013/071698;

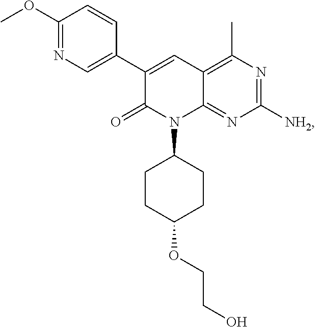 [SMILES: COc1ccc(-c2cc3c(C)nc(N)nc3n([C@H]3CC[C@H](OCCO)CC3)c2=O)cn1]

as described in Yu, C. et al., US2014/038991;

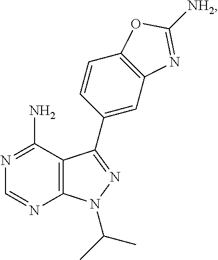 [SMILES: CC(C)n1nc(-c2ccc3oc(N)nc3c2)c2c(N)ncnc21]

as described in Conejo-Garcia, J. R. et al., WO 2017/062426;

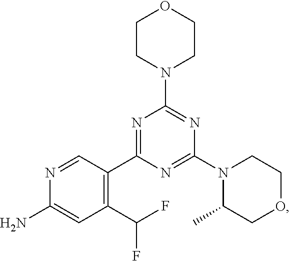 [SMILES: C[C@H]1COCCN1c1nc(-c2cnc(N)cc2C(F)F)nc(N2CCOCC2)n1]

as described in Rageot, D. et al., US2019/284178;

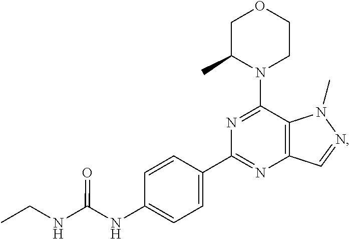 [SMILES: CCNC(=O)Nc1ccc(-c2nc(N3CCOC[C@@H]3C)c3c(cnn3C)n2)cc1]

as described in Pei, Z. et al., WO 2011/058025;

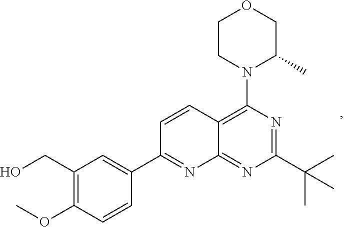 [SMILES: COc1ccc(-c2ccc3c(N4CCOC[C@@H]4C)nc(C(C)(C)C)nc3n2)cc1CO]

as described in Li, X. et al., WO 2013/016999;

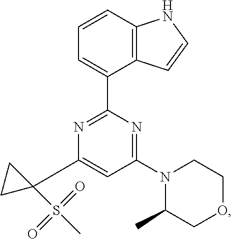 [SMILES: C[C@@H]1COCCN1c1cc(C2(S(C)(=O)=O)CC2)nc(-c2cccc3[nH]ccc23)n1]

as described in Foote, K. M. et al., WO 2010/073034;

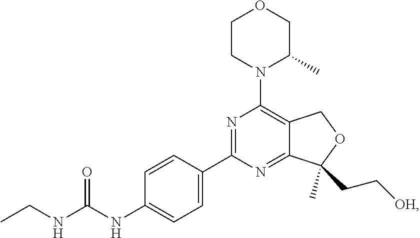 [SMILES: CCNC(=O)Nc1ccc(-c2nc(N3CCOC[C@@H]3C)c3c(n2)[C@](C)(CCO)OC3)cc1]

as described in Bergeron, P. et al., WO

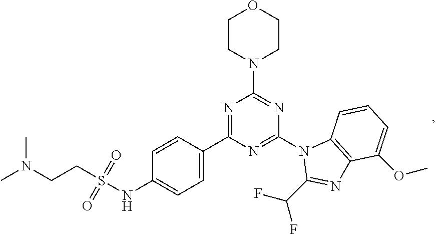

as described in Chen, L. et al., CN106806948.

In certain embodiments, the EPL is a radical of one of the above compounds, which is attached to L through a modifiable oxygen, nitrogen, or carbon atom.

In certain embodiments, the EPL has the following formula:

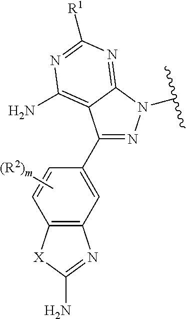

wherein:
R¹ and each R² represent independently for each occurrence halo, C₁-C₆ alkyl, C₁-C₆ haloalkyl, C₃-C₆ cycloalkyl, hydroxyl, C₁-C₆ alkoxy, or cyano;
R³ is hydrogen, C₁-C₆ alkyl, or C₃-C₆ cycloalkyl;
X is O, S, or N(R³); and
m is 0, 1, 2, or 3.

In certain embodiments, the EPL has the following formula:

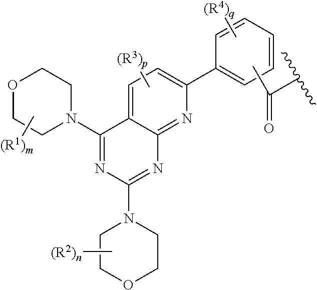

wherein:
R¹ and R² each represent independently for each occurrence halo, C₁-C₆ alkyl, C₁-C₆ haloalkyl, or C₃-C₆ cycloalkyl;
R³ and R⁴ each represent independently for each occurrence halo, C₁-C₆ alkyl, C₁-C₆ haloalkyl, C₃-C₆ cycloalkyl, hydroxyl, C₁-C₆ alkoxy, or cyano;
m, n, and q each represent independently 0, 1, 2, or 3; and
p is 0, 1, or 2.

In certain embodiments, the EPL is one of the following:

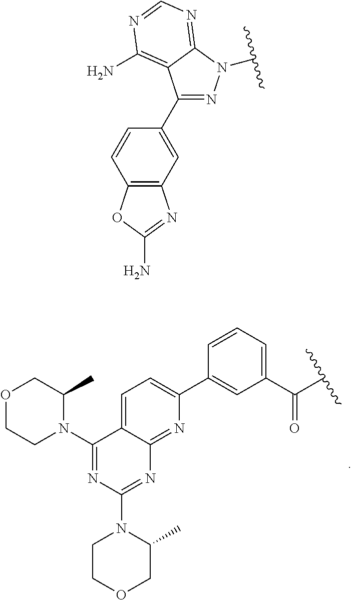 [SMILES: *n1nc(-c2ccc3oc(N)nc3c2)c2c(N)ncnc21 ; *C(=O)c1cccc(-c2ccc3c(N4CCOC[C@H]4C)nc(N4CCOC[C@H]4C)nc3n2)c1]

### B. Moiety for PLK1

In certain embodiments, the EPL is a moiety that binds to Polo Like Kinase 1 (PLK1). Exemplary compounds that bind to PLK1 are reported in the literature, including:

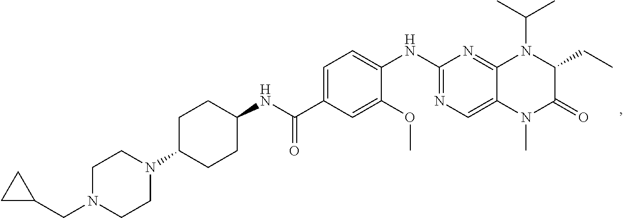 [SMILES: CC[C@@H]1C(=O)N(C)c2cnc(Nc3ccc(C(=O)N[C@H]4CC[C@H](N5CCN(CC6CC6)CC5)CC4)cc3OC)nc2N1C(C)C]

as described by Grauert, M., et al. in WO2004/076454;

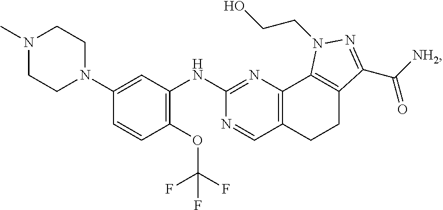 [SMILES: CN1CCN(c2ccc(OC(F)(F)F)c(Nc3ncc4c(n3)-c3c(c(C(N)=O)nn3CCO)CC4)c2)CC1]

as described by Caruso, M, et al. in WO2008/074788;

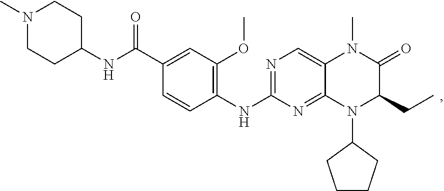 [SMILES: CC[C@@H]1C(=O)N(C)c2cnc(Nc3ccc(C(=O)NC4CCN(C)CC4)cc3OC)nc2N1C1CCCC1]

as described Grauert, M., et al. in WO2004/076454;

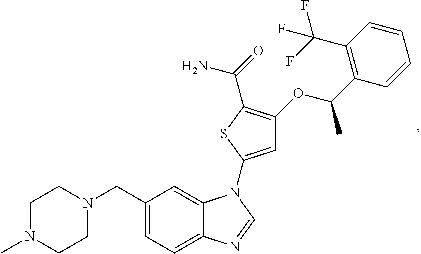 [SMILES: C[C@@H](Oc1cc(-n2cnc3ccc(CN4CCN(C)CC4)cc32)sc1C(N)=O)c1ccccc1C(F)(F)F]

N as described by Cheung, M., et al. in WO2007/030361;

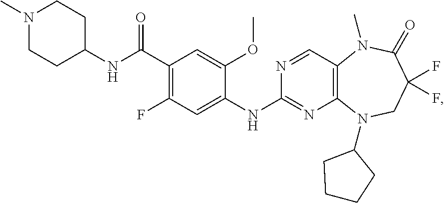 [SMILES: COc1cc(C(=O)NC2CCN(C)CC2)c(F)cc1Nc1ncc2c(n1)N(C1CCCC1)CC(F)(F)C(=O)N2C]

as described by Cao, S. X., et al. in WO2009/042711;

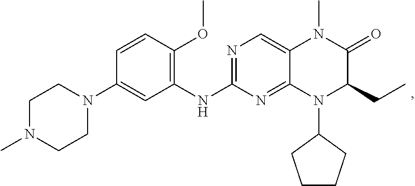 [SMILES: CC[C@@H]1C(=O)N(C)c2cnc(Nc3cc(N4CCN(C)CC4)ccc3OC)nc2N1C1CCCC1]

as described by Caruso, M., et al. in WO2009/071480;

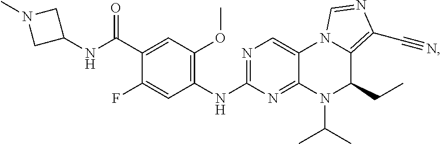 [SMILES: CC[C@@H]1c2c(C#N)ncn2-c2cnc(Nc3cc(F)c(C(=O)NC4CN(C)C4)cc3OC)nc2N1C(C)C]

as described by Kiryanov, A., et al. in Bioorg Med Chem Lett 2017, 27(5): 1311;

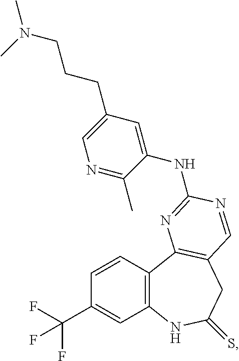 [SMILES: Cc1ncc(CCCN(C)C)cc1Nc1ncc2c(n1)-c1ccc(C(F)(F)F)cc1NC(=S)C2]

as described by Bharathan, I. T., et al. in WO2010/065134.

In certain embodiments, the EPL is a radical of one of the above compounds, which is attached to L through a modifiable oxygen, nitrogen, or carbon atom.

In certain embodiments, the EPL has the following formula:

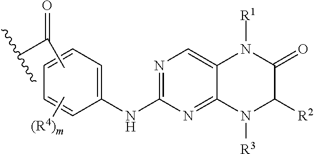

wherein:
R¹ and R² each represent independently C₁-C₆ alkyl, C₁-C₆ haloalkyl, or hydrogen;
R³ is C₃-C₆ cycloalkyl, C₁-C₆ alkyl, C₁-C₆ haloalkyl, or hydrogen;
R⁴ represents independently for each occurrence halo, C₁-C₆ alkyl, C₁-C₆ haloalkyl, C₃-C₆ cycloalkyl, hydroxyl, C₁-C₆ alkoxy, or cyano; and
m is 0, 1, 2, or 3.

In certain embodiments, the EPL

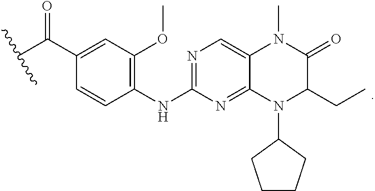 [SMILES: *C(=O)c1ccc(Nc2ncc3c(n2)N(C2CCCC2)C(CC)C(=O)N3C)c(OC)c1]

In certain embodiments, the EPL is

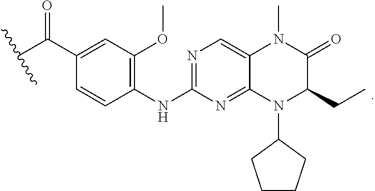 [SMILES: *C(=O)c1ccc(Nc2ncc3c(n2)N(C2CCCC2)[C@H](CC)C(=O)N3C)c(OC)c1]

In certain embodiments, the EPL has the following formula:

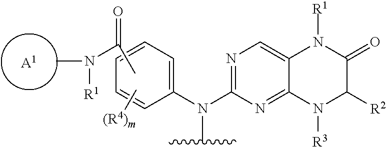

wherein:
R¹ and R² each represent independently for each occurrence C₁-C₆ alkyl, C₁-C₆ haloalkyl, or hydrogen;
R³ is C₃-C₆ cycloalkyl, C₁-C₆ alkyl, C₁-C₆ haloalkyl, or hydrogen;
R⁴ represents independently for each occurrence halo, C₁-C₆ alkyl, C₁-C₆ haloalkyl, C₃-C₆ cycloalkyl, hydroxyl, C₁-C₆ alkoxy, or cyano;
A¹ is a 3-7 membered saturated heterocyclyl containing 1, 2, or 3 heteroatoms independently selected from nitrogen, oxygen, and sulfur, wherein the heterocyclyl is optionally substituted with 1 or 2 occurrences of R⁴; and
m is 0, 1, 2, or 3.

In certain embodiments, the EPL has the following formula:

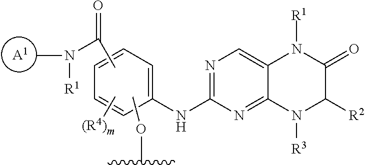

wherein:
R¹ and R² each represent independently for each occurrence C₁-C₆ alkyl, C₁-C₆ haloalkyl, or hydrogen;
R³ is C₃-C₆ cycloalkyl, C₁-C₆ alkyl, C₁-C₆ haloalkyl, or hydrogen;
R⁴ represents independently for each occurrence halo, C₁-C₆ alkyl, C₁-C₆ haloalkyl, C₃-C₆ cycloalkyl, hydroxyl, C₁-C₆ alkoxy, or cyano;
A¹ is a 3-7 membered saturated heterocyclyl containing 1, 2, or 3 heteroatoms independently selected from nitrogen, oxygen, and sulfur, wherein the heterocyclyl is optionally substituted with 1 or 2 occurrences of R⁴; and
m is 0, 1, 2, or 3.

In certain embodiments, the EPL is one of the following:

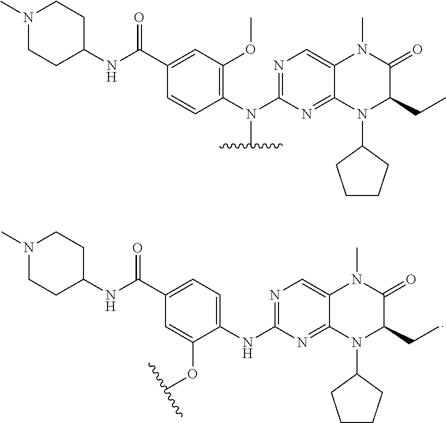

### C. Moiety for CDK1

In certain embodiments, the EPL is a moiety that binds to Cyclin-Dependent Kinase 1 (CDK1). Exemplary compounds that bind to CDK1 are reported in the literature, including:

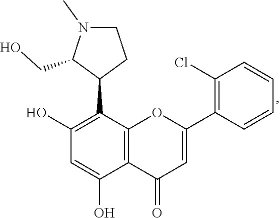 [SMILES: CN1CC[C@@H](c2c(O)cc(O)c3c(=O)cc(-c4ccccc4Cl)oc23)[C@@H]1CO]

as described by Sivakumar, M., et al. in WO2007/148158;

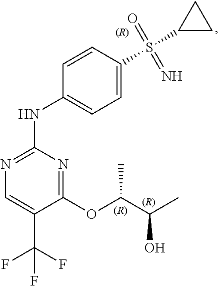

as described by Lucking, U., et al. in WO2005/037800;

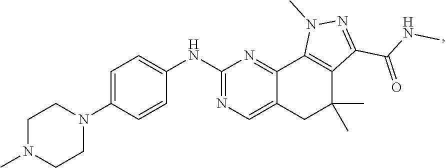

as described by D'Alessio, R., et al. in WO2004/104007;

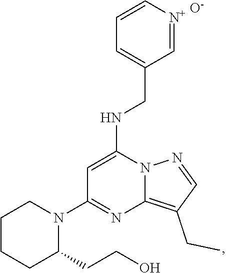

as described by Guzi, T. J., et al. in WO2005/077954;

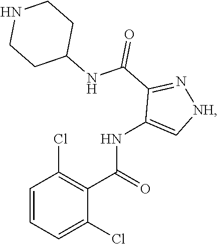 [SMILES: O=C(NC1CCNCC1)c1n[nH]cc1NC(=O)c1c(Cl)cccc1Cl]

as described by Wyatt, P. G., et al. in WO2005/012256;

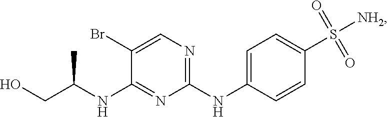 [SMILES: C[C@H](CO)Nc1nc(Nc2ccc(S(N)(=O)=O)cc2)ncc1Br]

as described by Brumby, T., et al. in WO2002/096888;

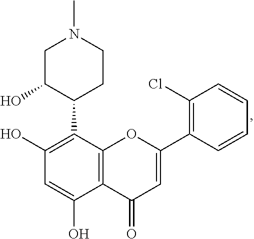 [SMILES: CN1CC[C@H](c2c(O)cc(O)c3c(=O)cc(-c4ccccc4Cl)oc23)[C@H](O)C1]

as described by Dumont, J. A., et al. in WO2000/044362;

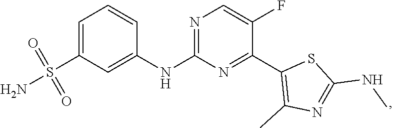 [SMILES: CNc1nc(C)c(-c2nc(Nc3cccc(S(N)(=O)=O)c3)ncc2F)s1]

as described by Wang, S., et al. in WO2013/156780;

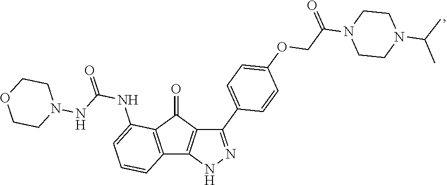 [SMILES: CC(C)N1CCN(C(=O)COc2ccc(-c3n[nH]c4c3C(=O)c3c(NC(=O)NN5CCOCC5)cccc3-4)cc2)CC1]

as described by Wang, Z., et al. in WO2004/092139;

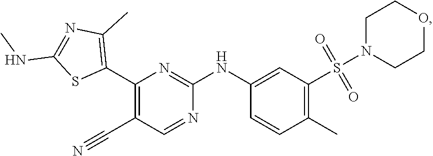 [SMILES: CNc1nc(C)c(-c2nc(Nc3ccc(C)c(S(=O)(=O)N4CCOCC4)c3)ncc2C#N)s1]

as described by Wang, S., et al. in WO2009/118567;

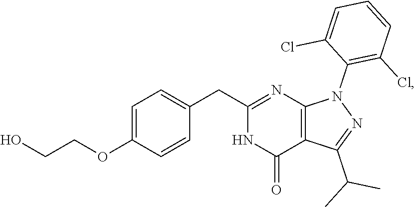 [SMILES: CC(C)c1nn(-c2c(Cl)cccc2Cl)c2nc(Cc3ccc(OCCO)cc3)[nH]c(=O)c12]

as described by Caligiuri, M., et al. in Chem Biol (London) 2005, 12(10): 1103.

In certain embodiments, the EPL is a radical of one of the above compounds, which is attached to L through a modifiable oxygen, nitrogen, or carbon atom.

In certain embodiments, the EPL has the following formula:

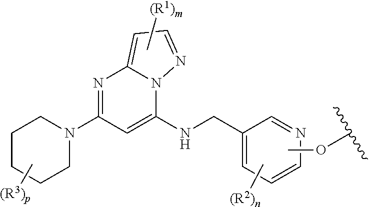

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

In certain embodiments, the EPL has the following formula:

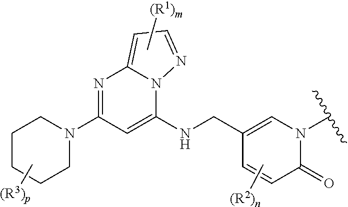

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

In certain embodiments, the EPL has the following formula:

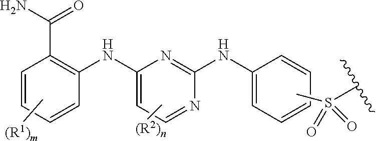

wherein:
R¹, R² and R³ each represent independently for each occurrence halo, C₁-C₆ alkyl, C₁-C₆ haloalkyl, C₃-C₆ cycloalkyl, hydroxyl, C₁-C₆ alkoxy, or cyano; m and p each represent independently 0, 1, or 2; and
n is 0, 1 or 2.

In certain embodiments, the EPL is one of the following:

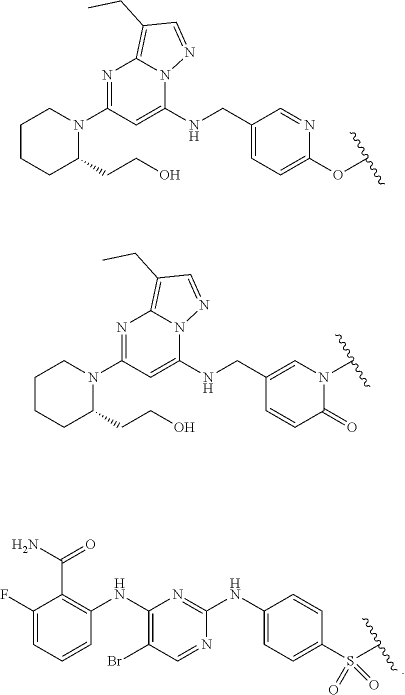 [SMILES: *Oc1ccc(CNc2cc(N3CCCC[C@H]3CCO)nc3c(CC)cnn23)cn1 ; *n1cc(CNc2cc(N3CCCC[C@H]3CCO)nc3c(CC)cnn23)ccc1=O ; *S(=O)(=O)c1ccc(Nc2ncc(Br)c(Nc3cccc(F)c3C(N)=O)n2)cc1]

In certain embodiments, the EPL is one of the following:

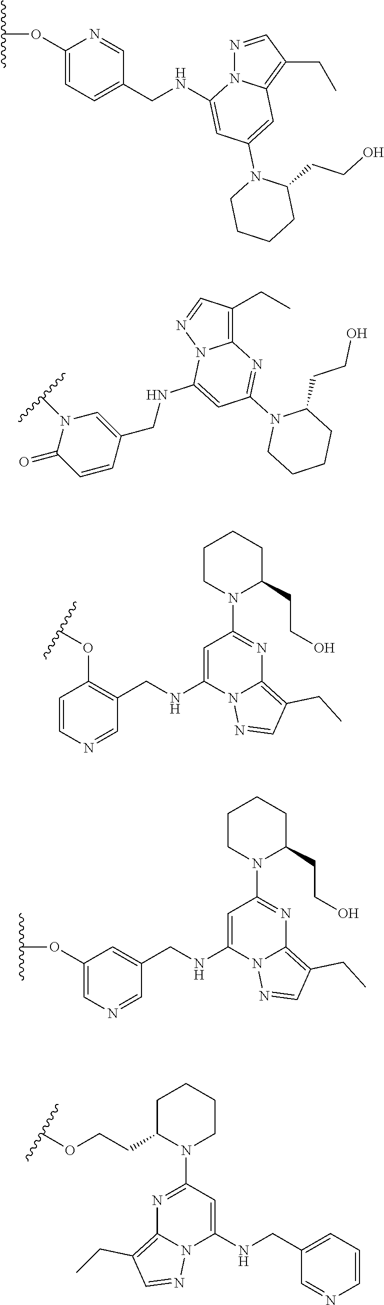 [SMILES: *Oc1ccc(CNc2cc(N3CCCC[C@H]3CCO)cc3c(CC)cnn23)cn1 ; *n1cc(CNc2cc(N3CCCC[C@H]3CCO)nc3c(CC)cnn23)ccc1=O ; *Oc1ccncc1CNc1cc(N2CCCC[C@H]2CCO)nc2c(CC)cnn12 ; *Oc1cncc(CNc2cc(N3CCCC[C@H]3CCO)nc3c(CC)cnn23)c1 ; *OCC[C@@H]1CCCCN1c1cc(NCc2cccnc2)n2ncc(CC)c2n1]

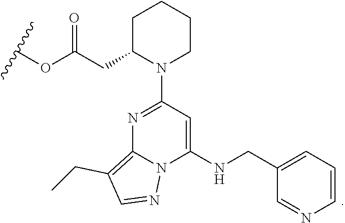 [SMILES: *OC(=O)C[C@@H]1CCCCN1c1cc(NCc2cccnc2)n2ncc(CC)c2n1]

### D. Moiety for CDK2

In certain embodiments, the EPL is a moiety that binds to Cyclin-Dependent Kinase 2 (CDK2). Exemplary compounds that bind to CDK2 are reported in the literature, including:

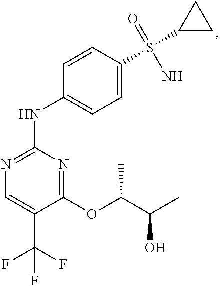

as described by Lucking, U., et al. in WO2005/037800;

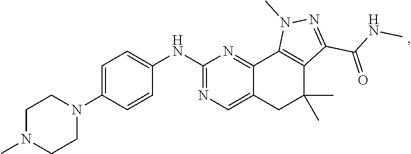

as described by D'Alessio, R., et al. in WO2004/104007;

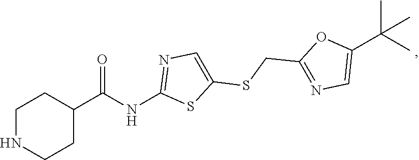 [SMILES: CC(C)(C)c1cnc(CSc2cnc(NC(=O)C3CCNCC3)s2)o1]

as described by Misra, R. N., et al. in WO2001/044242;

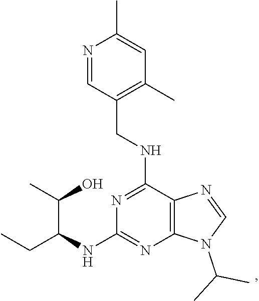 [SMILES: CC[C@H](Nc1nc(NCc2cnc(C)cc2C)c2ncn(C(C)C)c2n1)[C@@H](C)O]

as described bySheldrake, P. W., et al. in WO2008/122767;

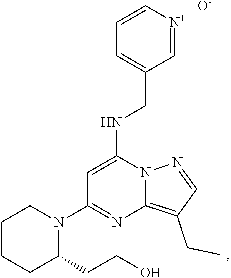

as described by Guzi, T. J., et al. in WO2005/077954;

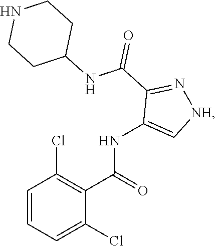 [SMILES: O=C(NC1CCNCC1)c1n[nH]cc1NC(=O)c1c(Cl)cccc1Cl]

as described by Wyatt, P. G., et al. in WO2005/012256;

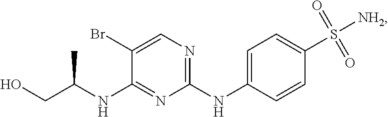 [SMILES: C[C@H](CO)Nc1nc(Nc2ccc(S(N)(=O)=O)cc2)ncc1Br]

as described by Brumby, T., et al. in WO2002/096888;

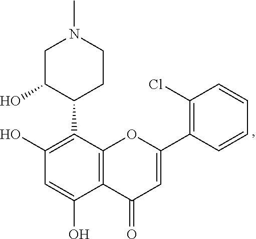 [SMILES: CN1CC[C@H](c2c(O)cc(O)c3c(=O)cc(-c4ccccc4Cl)oc23)[C@H](O)C1]

as described by Dumont, J. A., et al. in WO2000/044362;

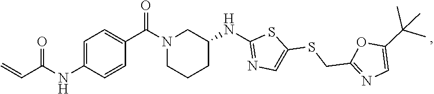 [SMILES: C=CC(=O)Nc1ccc(C(=O)N2CCC[C@@H](Nc3ncc(SCc4ncc(C(C)(C)C)o4)s3)C2)cc1]

as described by Hao, M., et al. in WO2017/044858.

In certain embodiments, the EPL is a radical of one of the above compounds, which is attached to L through a modifiable oxygen, nitrogen, or carbon atom.

In certain embodiments, the EPL has the following formula:

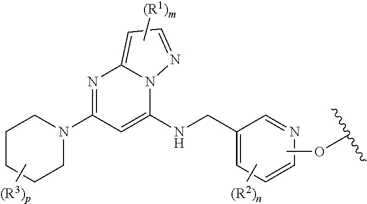

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

In certain embodiments, the EPL has the following formula:

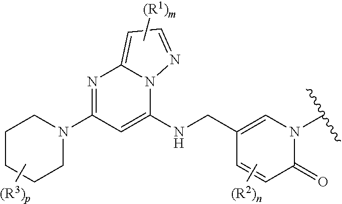

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

In certain embodiments, the EPL is one of the following:

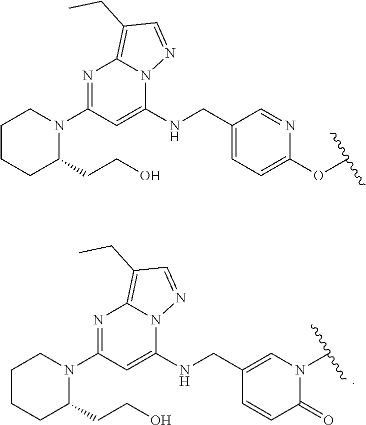 [SMILES: *Oc1ccc(CNc2cc(N3CCCC[C@H]3CCO)nc3c(CC)cnn23)cn1 ; *n1cc(CNc2cc(N3CCCC[C@H]3CCO)nc3c(CC)cnn23)ccc1=O]

### E Moiety for CDK9

In certain embodiments, the EPL is a moiety that binds to Cyclin-Dependent Kinase 9 (CDK9). Exemplary compounds that bind to CDK9 are reported in the literature, including:

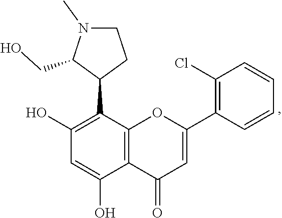 [SMILES: CN1CC[C@@H](c2c(O)cc(O)c3c(=O)cc(-c4ccccc4Cl)oc23)[C@@H]1CO]

as described by Sivakumar, M., et al. in WO2007/148158;

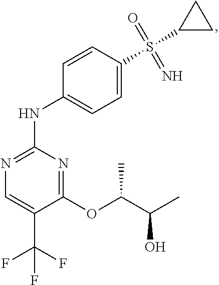 [SMILES: C[C@@H](O)[C@@H](C)Oc1nc(Nc2ccc([S@](=N)(=O)C3CC3)cc2)ncc1C(F)(F)F]

as described by Lucking, U., et al. in WO2005/037800;

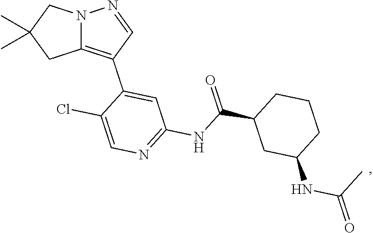 [SMILES: CC(=O)N[C@@H]1CCC[C@H](C(=O)Nc2cc(-c3cnn4c3CC(C)(C)C4)c(Cl)cn2)C1]

as described by Pike, K. G., et al. in WO2017001354;

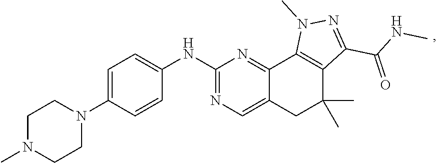

as described by D'Alessio, R., et al. in WO2004/104007;

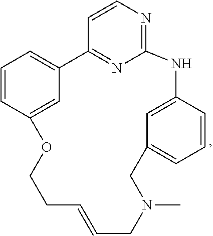 [SMILES: CN1C/C=C/CCOc2cccc(c2)-c2ccnc(n2)Nc2cccc(c2)C1]

as described by Blanchard, S., et al. in WO2007/058628;

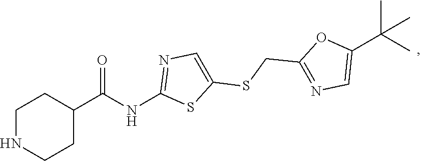 [SMILES: CC(C)(C)c1cnc(CSc2cnc(NC(=O)C3CCNCC3)s2)o1]

as described by Misra, R. N., et al. in WO2001/044242;

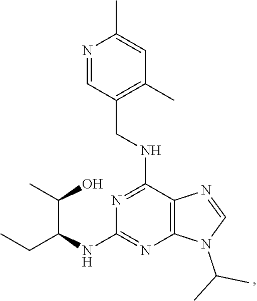 [SMILES: CC[C@H](Nc1nc(NCc2cnc(C)cc2C)c2ncn(C(C)C)c2n1)[C@@H](C)O]

as described by Sheldrake, P. W. et al. in WO2008/122767;

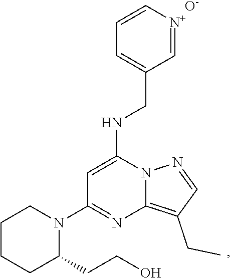

as described by Guzi, T. J., et al. in WO2005/077954;

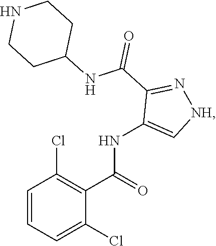 [SMILES: O=C(NC1CCNCC1)c1n[nH]cc1NC(=O)c1c(Cl)cccc1Cl]

as described by Wyatt, P. G., et al. in WO2005/012256;

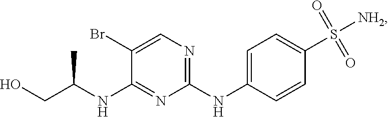 [SMILES: C[C@H](CO)Nc1nc(Nc2ccc(S(N)(=O)=O)cc2)ncc1Br]

as described by Brumby, T., et al. in WO2002/096888;

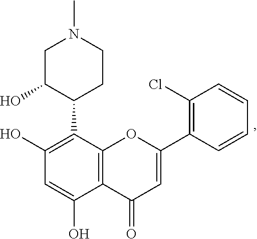 [SMILES: CN1CC[C@H](c2c(O)cc(O)c3c(=O)cc(-c4ccccc4Cl)oc23)[C@H](O)C1]

as described by Dumont, J. A., et al. in WO2000/044362;

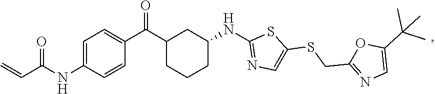 [SMILES: C=CC(=O)Nc1ccc(C(=O)C2CCC[C@@H](Nc3ncc(SCc4ncc(C(C)(C)C)o4)s3)C2)cc1]

as described by Hao, M., et al. in WO2017/044858;

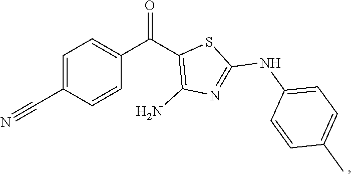 [SMILES: Cc1ccc(Nc2nc(N)c(C(=O)c3ccc(C#N)cc3)s2)cc1]

as described by Smith, C. D., et al. in WO2018/089902;

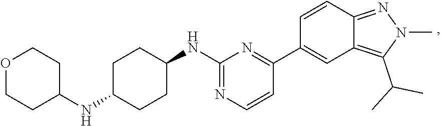 [SMILES: CC(C)c1c2cc(-c3ccnc(N[C@H]4CC[C@H](NC5CCOCC5)CC4)n3)ccc2nn1C]

as described by Gao, Q., et al. in CN105111191.

In certain embodiments, the EPL is a radical of one of the above compounds, which is attached to L through a modifiable oxygen, nitrogen, or carbon atom.

In certain embodiments, the EPL has the following formula:

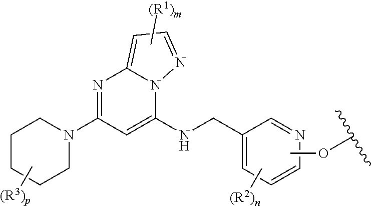

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

In certain embodiments, the EPL has the following formula:

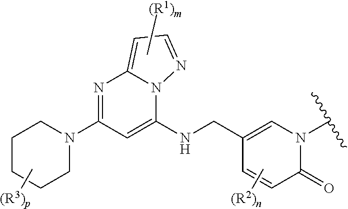

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

In certain embodiments, the EPL is one of the following:

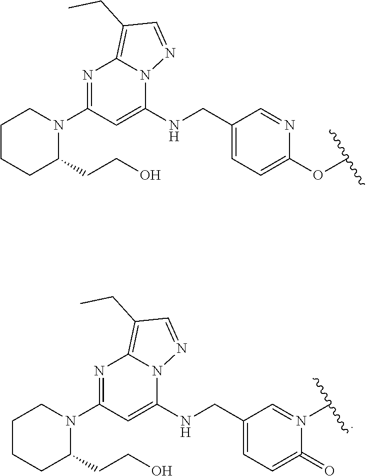

### F. Moiety for BRD4

In certain embodiments, the EPL is a moiety that binds to bromodomain-containing protein 4 (BRD4). Exemplary compounds that bind to BRD4 are reported in the literature, including:

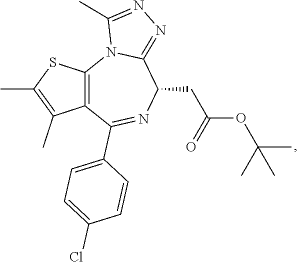 [SMILES: Cc1sc2c(c1C)C(c1ccc(Cl)cc1)=N[C@@H](CC(=O)OC(C)(C)C)c1nnc(C)n1-2]

as described by Bradner, J. E., et al. in WO 2011/143669;

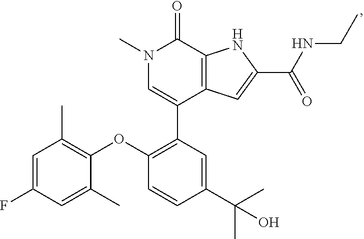 [SMILES: CCNC(=O)c1cc2c(-c3cc(C(C)(C)O)ccc3Oc3c(C)cc(F)cc3C)cn(C)c(=O)c2[nH]1]

as described by Fidanze, S. D., et al. in WO 2017/177955;

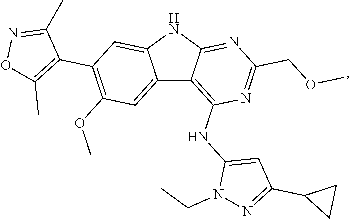 [SMILES: CCn1nc(C2CC2)cc1Nc1nc(COC)nc2[nH]c3cc(-c4c(C)noc4C)c(OC)cc3c12]

as described by Wang, S., et al. in WO 2016/138332;

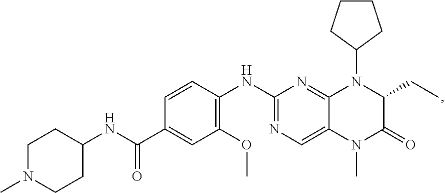 [SMILES: CC[C@@H]1C(=O)N(C)c2cnc(Nc3ccc(C(=O)NC4CCN(C)CC4)cc3OC)nc2N1C1CCCC1]

as described by Chen, L., et al. in ACS Med Chem Lett 2015, vol. 6(7), page 764;

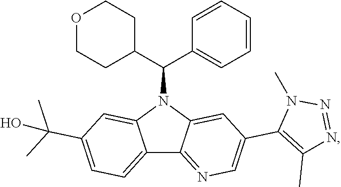 [SMILES: Cc1nnn(C)c1-c1cnc2c3ccc(C(C)(C)O)cc3n([C@H](c3ccccc3)C3CCOCC3)c2c1]

N as described by Norris, D. J., et al. in WO 2015/100282.

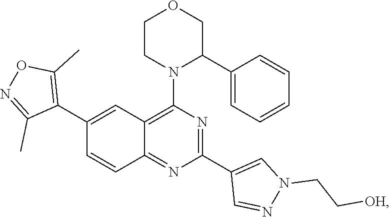 [SMILES: Cc1noc(C)c1-c1ccc2nc(-c3cnn(CCO)c3)nc(N3CCOCC3c3ccccc3)c2c1]

as described by Yang, S. M., et al. in Bioorg Med Chem Lett 2018, vol. 28(21), page 3483;

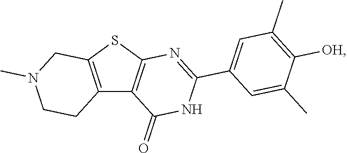 [SMILES: Cc1cc(-c2nc3sc4c(c3c(=O)[nH]2)CCN(C)C4)cc(C)c1O]

as described by Ouyang, L., et al. in J Med Chem 2017, vol. 60(24), page 9990;

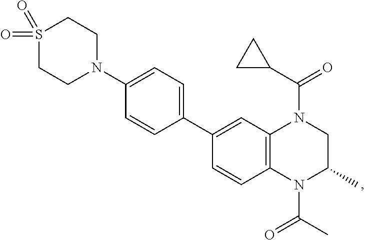 [SMILES: CC(=O)N1c2ccc(-c3ccc(N4CCS(=O)(=O)CC4)cc3)cc2N(C(=O)C2CC2)C[C@@H]1C]

as described by Millan, D. S., et al. in ACS Med Chem Lett 2017, vol. 8(8), page 847;

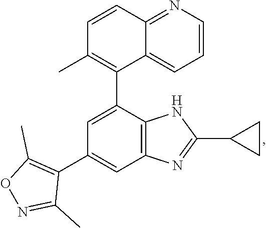 [SMILES: Cc1ccc2ncccc2c1-c1cc(-c2c(C)noc2C)cc2nc(C3CC3)[nH]c12]

as described by Aktoudianakis, E, et al. in WO 2014/182929;

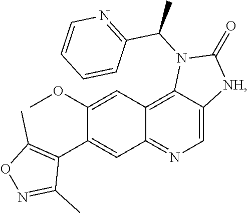 [SMILES: COc1cc2c(cc1-c1c(C)noc1C)ncc1[nH]c(=O)n([C@H](C)c3ccccn3)c12]

as described by Chekler, E. L. P., et al. in WO 2017/037567;

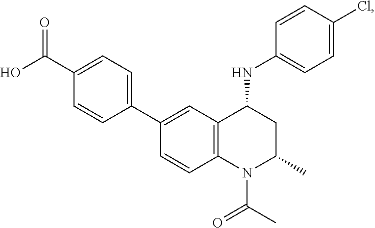 [SMILES: CC(=O)N1c2ccc(-c3ccc(C(=O)O)cc3)cc2[C@H](Nc2ccc(Cl)cc2)C[C@@H]1C]

as described by Demont, E. H., et al. in WO 2011/054848;

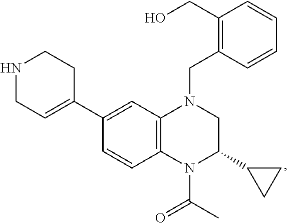 [SMILES: CC(=O)N1c2ccc(C3=CCNCC3)cc2N(Cc2ccccc2CO)C[C@@H]1C1CC1]

as described in Law, R. P., et al., et al. in J Med Chem 2018, vol. 61(10), page 4317;

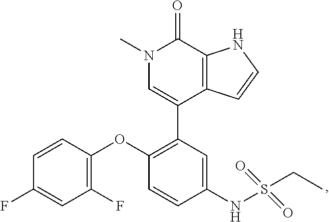 [SMILES: CCS(=O)(=O)Nc1ccc(Oc2ccc(F)cc2F)c(-c2cn(C)c(=O)c3[nH]ccc23)c1]

as described by Hu, Y., et al., et al. in WO 2018/086605;

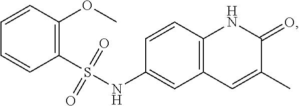 [SMILES: COc1ccccc1S(=O)(=O)Nc1ccc2[nH]c(=O)c(C)cc2c1]

as described by Fish, P. V., et al. in WO 2013/027168;

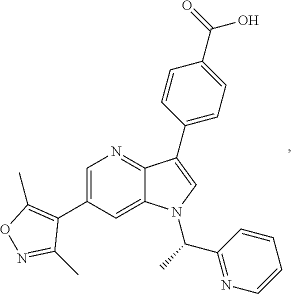 [SMILES: Cc1noc(C)c1-c1cnc2c(-c3ccc(C(=O)O)cc3)cn([C@@H](C)c3ccccn3)c2c1]

as described by Ozer, H. G., et al. in Cancer Discov 2018, vol. 8(4), page 458;

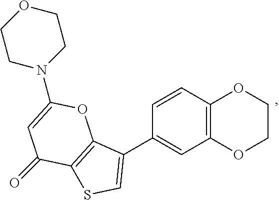 [SMILES: O=c1cc(N2CCOCC2)oc2c(-c3ccc4c(c3)OCCO4)csc12]

as described by Andrews, F. H., et al. in Proc Natl Acad Sci USA (PNAS) 2017, vol. 114(7), page E1072;

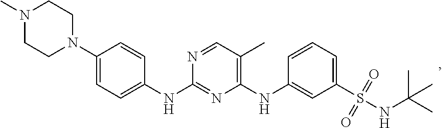 [SMILES: Cc1cnc(Nc2ccc(N3CCN(C)CC3)cc2)nc1Nc1cccc(S(=O)(=O)NC(C)(C)C)c1]

as described by Embe, et al. in ACS Chem Biol 2014, vol. 9(5), page 1160;

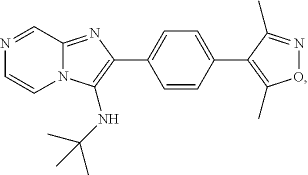 [SMILES: Cc1noc(C)c1-c1ccc(-c2nc3cnccn3c2NC(C)(C)C)cc1]

as described by Marineau, J. J., et al. in WO 2015/013635;

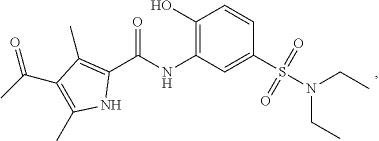 [SMILES: CCN(CC)S(=O)(=O)c1ccc(O)c(NC(=O)c2[nH]c(C)c(C(C)=O)c2C)c1]

as described by Huegle, M., et al. in J Med Chem 2016, vol. 59(4), page 1518;

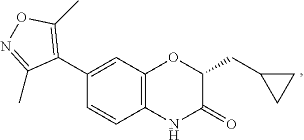 [SMILES: Cc1noc(C)c1-c1ccc2c(c1)O[C@H](CC1CC1)C(=O)N2]

as described by Xue, X. Q., et al. in Eur J Med Chem 2018, vol. 152, page 542;

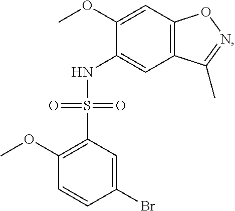 [SMILES: COc1cc2onc(C)c2cc1NS(=O)(=O)c1cc(Br)ccc1OC]

as described by Zhang, M., et al. in J Med Chem 2018, vol. 61(7), page 3037;

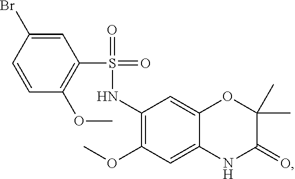 [SMILES: COc1cc2c(cc1NS(=O)(=O)c1cc(Br)ccc1OC)OC(C)(C)C(=O)N2]

as described by Xiang. Q., et al. in ACS Med Chem Lett 2018, vol. 9(3), page 262

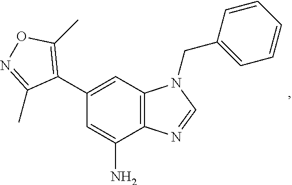 [SMILES: Cc1noc(C)c1-c1cc(N)c2ncn(Cc3ccccc3)c2c1]

as described by Kharenko, O. A., et al. in J Med Chem 2018, vol. 61(18), page 8202.

In certain embodiments, the EPL is a radical of one of the above compounds, which is attached to L through a modifiable oxygen, nitrogen, or carbon atom.

In certain embodiments, the EPL has the following formula:

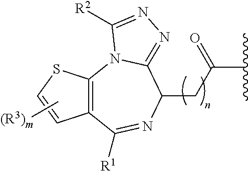

wherein:
R¹ is phenyl, C₃-C₈ cycloalkyl, or 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano;
R² and each R³ represent independently for each occurrence halo, C₁-C₆ alkyl, C₁-C₆ haloalkyl, C₃-C₆ cycloalkyl, hydroxyl, C₁-C₆ alkoxy, or cyano;
m is 0, 1, or 2; and
n is 0, 1, 2, 3, or 4.

In certain embodiments, the EPL is

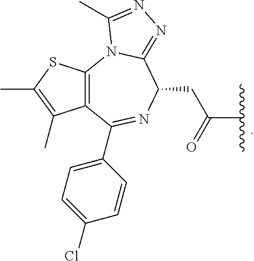 [SMILES: *C(=O)C[C@@H]1N=C(c2ccc(Cl)cc2)c2c(sc(C)c2C)-n2c(C)nnc21]

### G. Moiety for AURKA

In certain embodiments, the EPL is a moiety that binds to Aurora Kinase A (AURKA). Exemplary compounds that bind to AURKA are reported in the literature, including:

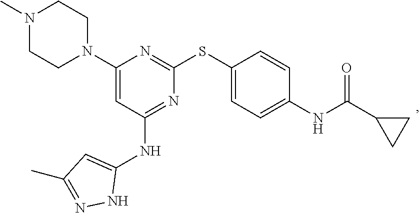 [SMILES: Cc1cc(Nc2cc(N3CCN(C)CC3)nc(Sc3ccc(NC(=O)C4CC4)cc3)n2)[nH]n1]

as described by Miller, A., et al. in WO2004/000833;

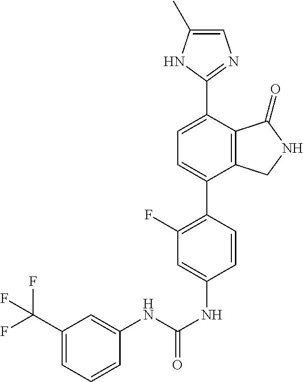 [SMILES: Cc1cnc(-c2ccc(-c3ccc(NC(=O)Nc4cccc(C(F)(F)F)c4)cc3F)c3c2C(=O)NC3)[nH]1]

as described by Hong, Y. R., et al. in WO2012/047017;

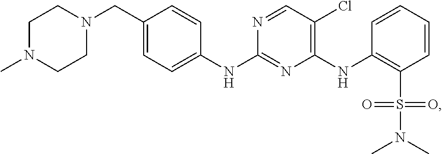 [SMILES: CN1CCN(Cc2ccc(Nc3ncc(Cl)c(Nc4ccccc4S(=O)(=O)N(C)C)n3)cc2)CC1]

as described by Bearss, D. J., et al. in WO2012/135800;

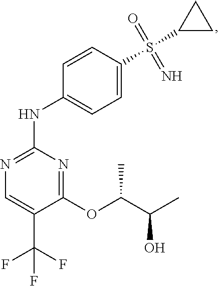 [SMILES: C[C@@H](O)[C@@H](C)Oc1nc(Nc2ccc([S@](=N)(=O)C3CC3)cc2)ncc1C(F)(F)F]

as described by Lucking, U., et al. in WO2005/037800;

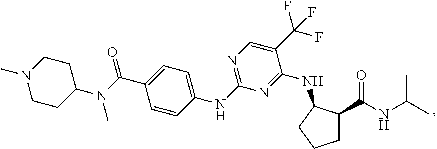 [SMILES: CC(C)NC(=O)[C@H]1CCC[C@H]1Nc1nc(Nc2ccc(C(=O)N(C)C3CCN(C)CC3)cc2)ncc1C(F)(F)F]

as described by Zahn, S. K., et al. in WO2007/132010;

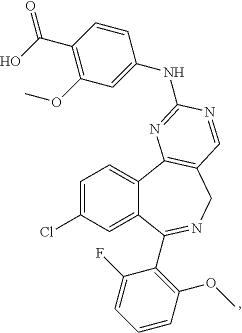 [SMILES: COc1cc(Nc2ncc3c(n2)-c2ccc(Cl)cc2C(c2c(F)cccc2OC)=NC3)ccc1C(=O)O]

as described by Claiborne, C. F., et al. in WO2005/111039;

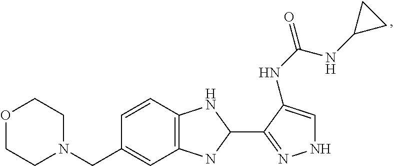 [SMILES: O=C(Nc1c[nH]nc1C1Nc2ccc(CN3CCOCC3)cc2N1)NC1CC1]

as described by Berdini, V., et al. in WO2006/070195;

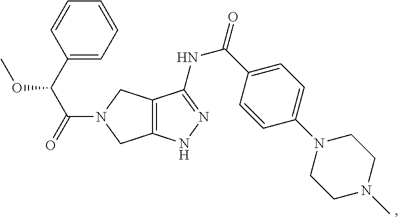

as described by Varasi, M., et al. in WO2005/005427;

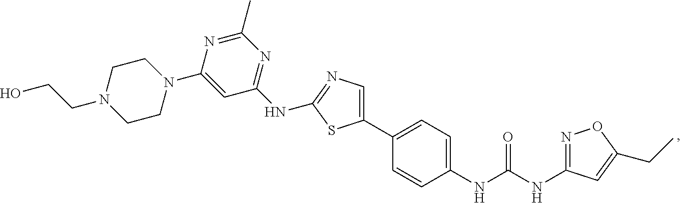 [SMILES: CCc1cc(NC(=O)Nc2ccc(-c3cnc(Nc4cc(N5CCN(CCO)CC5)nc(C)n4)s3)cc2)no1]

as described by Jiaang, W.-T., et al. in US2012/225880;

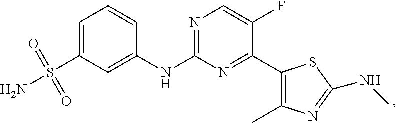 [SMILES: CNc1nc(C)c(-c2nc(Nc3cccc(S(N)(=O)=O)c3)ncc2F)s1]

as described by Wang, S., et al. in WO2013/156780;

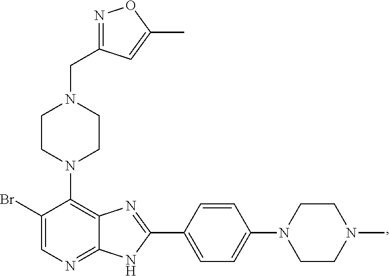 [SMILES: Cc1cc(CN2CCN(c3c(Br)cnc4[nH]c(-c5ccc(N6CCN(C)CC6)cc5)nc34)CC2)no1]

as described by Bavetsias, V., et al. in WO2007/072017;

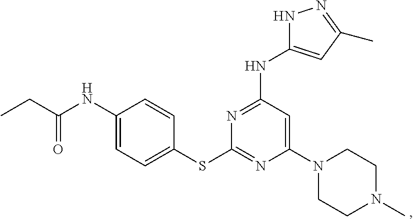 [SMILES: CCC(=O)Nc1ccc(Sc2nc(Nc3cc(C)n[nH]3)cc(N3CCN(C)CC3)n2)cc1]

as described by Miller, A., et al. in WO2004/000833;

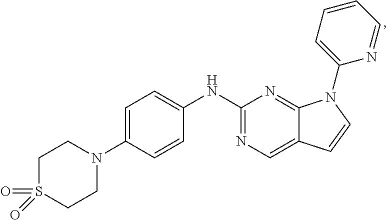 [SMILES: O=S1(=O)CCN(c2ccc(Nc3ncc4ccn(-c5ccccn5)c4n3)cc2)CC1]

as described by Moriarty, K. J., et al. in Bioorg Med Chem Lett 2006, 16(22): 5778.

In certain embodiments, the EPL is a radical of one of the above compounds, which is attached to L through a modifiable oxygen, nitrogen, or carbon atom.

In certain embodiments, the EPL has the following formula:

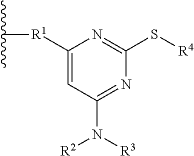

wherein:
R¹ is 4-7 membered, saturated heterocyclylene containing 1, 2, or 3 heteroatoms independently selected from nitrogen, oxygen, and sulfur;
R² is a 5-6 membered heteroaryl containing 1, 2, or 3 heteroatoms independently selected from nitrogen, oxygen, and sulfur; wherein the heteroaryl is optionally substituted with 1 or 2 substituents independently selected from halo, C₁-C₆ alkyl, C₁-C₆ haloalkyl, C₃-C₆ cycloalkyl, hydroxyl, C₁-C₆ alkoxy, or cyano;
R³ represents independently for each occurrence H or C₁-C₆ alkyl; and
R⁴ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from —N(R³)C(O)—(C₃-C₆ cycloalkyl), —N(R³)C(O)—(C₁-C₆ alkyl), halo, C₁-C₆ alkyl, C₁-C₆ haloalkyl, C₃-C₆ cycloalkyl, hydroxyl, C₁-C₆ alkoxy, or cyano.

In certain embodiments, the EPL has the following formula:

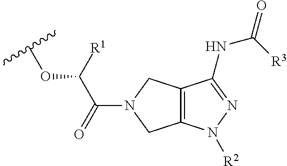

wherein:
R¹ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano.
R² is -(phenylene)-(4-7 membered, saturated heterocyclyl containing 1, 2, or 3 heteroatoms independently selected from nitrogen, oxygen, and sulfur); wherein the heterocyclyl is optionally substituted with 1 or 2 substituents independently selected from halo, C₁-C₆ alkyl, C₁-C₆ haloalkyl, C₃-C₆ cycloalkyl, hydroxyl, C₁-C₆ alkoxy, or cyano; and
R³ represents independently for each occurrence H or C₁-C₆ alkyl.

In certain embodiments, the EPL is one of the following:

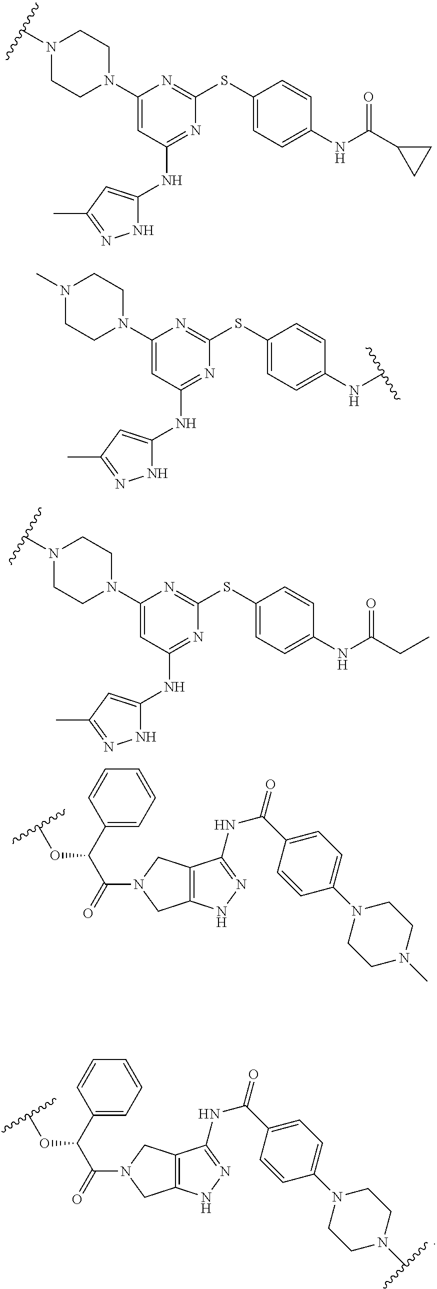

### H. Moiety for AURKB

In certain embodiments, the EPL is a moiety that binds to Aurora Kinase B (AURKB). Exemplary compounds that bind to AURKB are reported in the literature, including:

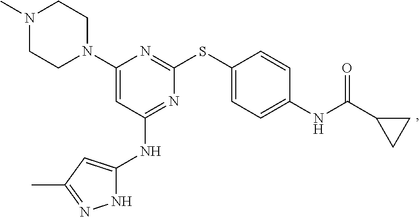 [SMILES: Cc1cc(Nc2cc(N3CCN(C)CC3)nc(Sc3ccc(NC(=O)C4CC4)cc3)n2)[nH]n1]

as described by Miller, A., et al. in WO2004/000833;

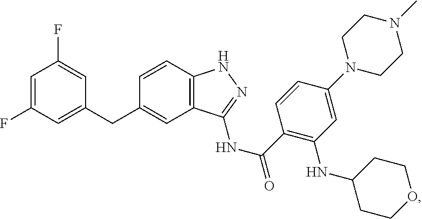 [SMILES: CN1CCN(c2ccc(C(=O)Nc3n[nH]c4ccc(Cc5cc(F)cc(F)c5)cc34)c(NC3CCOCC3)c2)CC1]

as described by Lombardi Borgia, A., et al. in WO2009/013126;

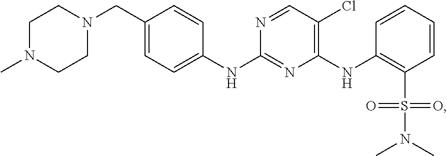 [SMILES: CN1CCN(Cc2ccc(Nc3ncc(Cl)c(Nc4ccccc4S(=O)(=O)N(C)C)n3)cc2)CC1]

as described by Bearss, D. J., et al. in WO2012/135800;

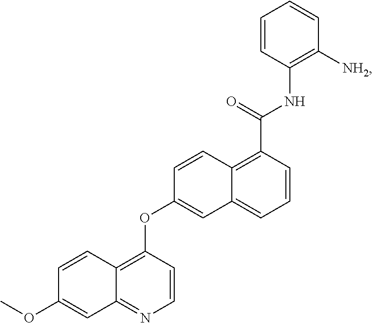 [SMILES: COc1ccc2c(Oc3ccc4c(C(=O)Nc5ccccc5N)cccc4c3)ccnc2c1]

as described by Lu, X., et al. in WO2010/139180;

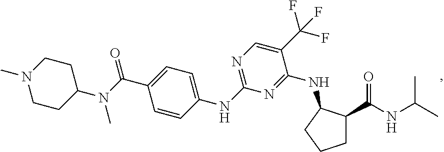 [SMILES: CC(C)NC(=O)[C@H]1CCC[C@H]1Nc1nc(Nc2ccc(C(=O)N(C)C3CCN(C)CC3)cc2)ncc1C(F)(F)F]

as described by Zahn, S. K., et al. in WO2007/132010;

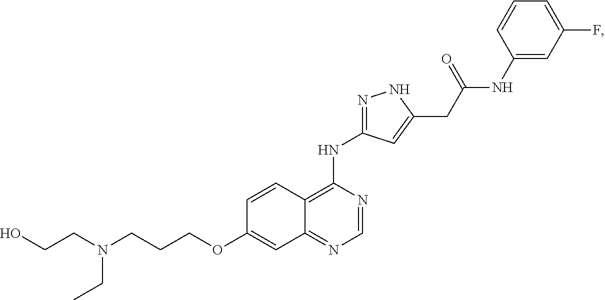 [SMILES: CCN(CCO)CCCOc1ccc2c(Nc3cc(CC(=O)Nc4cccc(F)c4)[nH]n3)ncnc2c1]

as described by Juhng F. H., et al. in WO2004/058781;

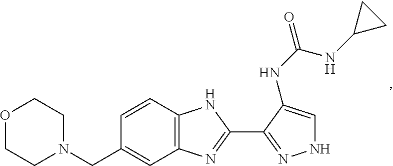 [SMILES: O=C(Nc1c[nH]nc1-c1nc2cc(CN3CCOCC3)ccc2[nH]1)NC1CC1]

as described by Berdini, V., et al. in WO2006/070195;

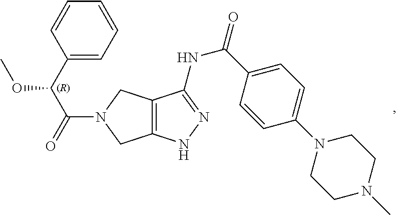

as described by Varasi, M., et al. in WO2005/005427;

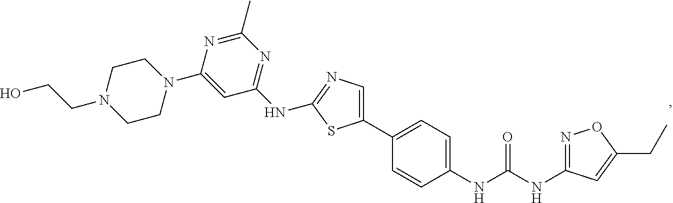 [SMILES: CCc1cc(NC(=O)Nc2ccc(-c3cnc(Nc4cc(N5CCN(CCO)CC5)nc(C)n4)s3)cc2)no1]

as described by Jiaang, W.-T., et al. in US2012/225880;

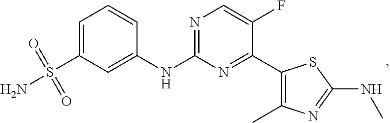 [SMILES: CNc1nc(C)c(-c2nc(Nc3cccc(S(N)(=O)=O)c3)ncc2F)s1]

as described by Wang, S., et al. in WO2013/156780;

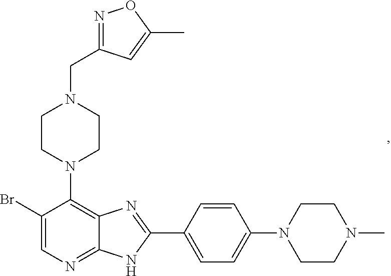 [SMILES: Cc1cc(CN2CCN(c3c(Br)cnc4[nH]c(-c5ccc(N6CCN(C)CC6)cc5)nc34)CC2)no1]

as described by Bavetsias, V., et al. in WO2007/072017;

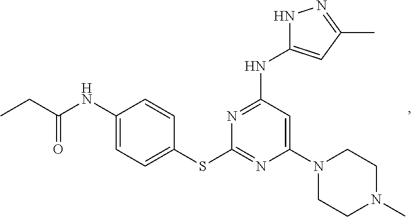 [SMILES: CCC(=O)Nc1ccc(Sc2nc(Nc3cc(C)n[nH]3)cc(N3CCN(C)CC3)n2)cc1]

as described by Miller, A., et al. in WO2004/000833;

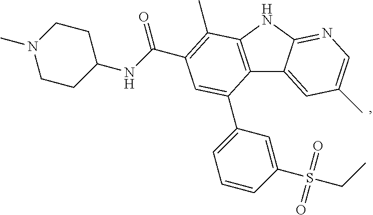 [SMILES: CCS(=O)(=O)c1cccc(-c2cc(C(=O)NC3CCN(C)CC3)c(C)c3[nH]c4ncc(C)cc4c23)c1]

as described by Brown, J W., et al. in WO2008/045834.

In certain embodiments, the EPL is a radical of one of the above compounds, which is attached to L through a modifiable oxygen, nitrogen, or carbon atom.

In certain embodiments, the EPL has the following formula:

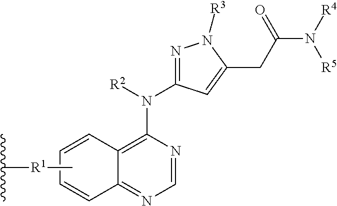

wherein:
R¹ is 4-10 membered heteroalkylene;
R², R³, and R⁵ are independently H or C₁-C₆ alkyl; and
R⁴ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano.

In certain embodiments, the EPL is one of the following:

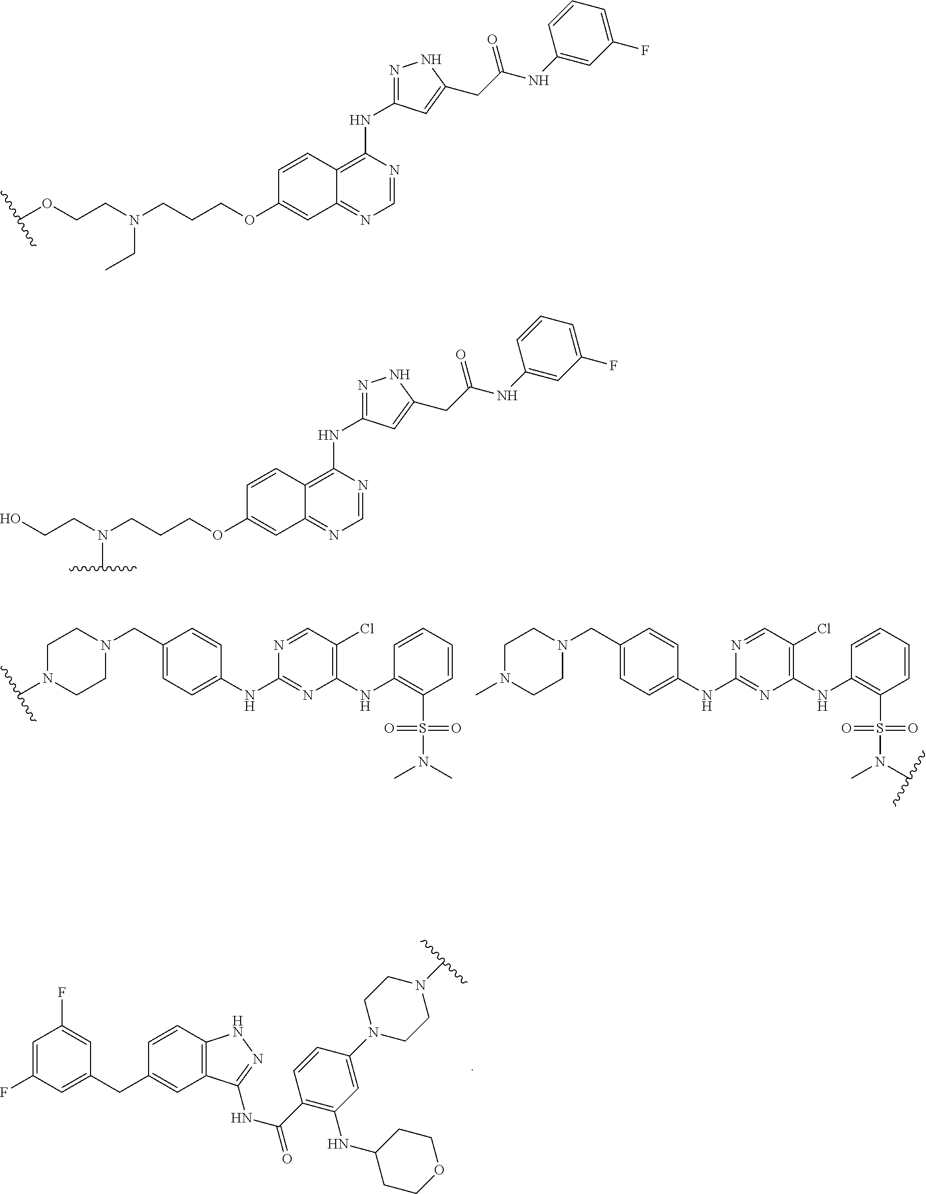

### L Moiety for MEK

In certain embodiments, the EPL is a moiety that binds to and inhibits Mitogen-activated protein kinase kinase (MEK). Exemplary compounds that bind to and inhibit MEK are reported in the literature, including:

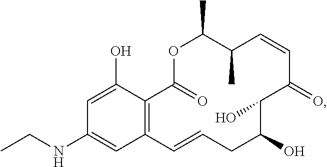 [SMILES: CCNc1cc(O)c2c(c1)/C=C/C[C@H](O)[C@H](O)C(=O)/C=C\[C@@H](C)[C@H](C)OC2=O]

as described in Narita, Y. et al., Mol Cancer Ther 2014, vol 13(4), page 823;

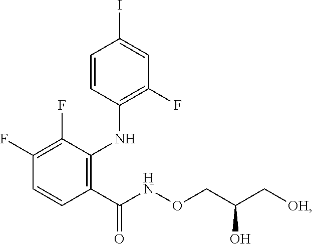 [SMILES: O=C(NOC[C@H](O)CO)c1ccc(F)c(F)c1Nc1ccc(I)cc1F]

as described in Berger, D. M. et al., Bioorg Med Chem 2008, vol 16(20), page 9202;

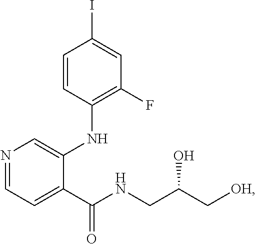 [SMILES: O=C(NC[C@H](O)CO)c1ccncc1Nc1ccc(I)cc1F]

as described in Haq, R. et al., WO 2014/138338;

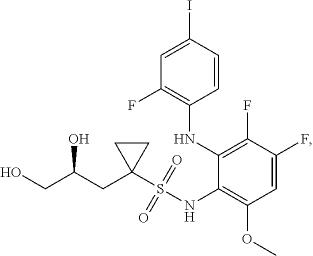 [SMILES: COc1cc(F)c(F)c(Nc2ccc(I)cc2F)c1NS(=O)(=O)C1(C[C@H](O)CO)CC1]

as described in Iverson, C. et al., Cancer Res 2009, vol 69(17), page 6839;

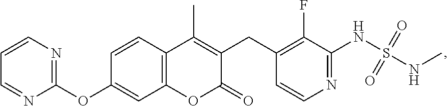 [SMILES: CNS(=O)(=O)Nc1nccc(Cc2c(C)c3ccc(Oc4ncccn4)cc3oc2=O)c1F]

as described in Aoki, T. et al., ACS Med Chem Lett 2014, vol 5(4), page 309;

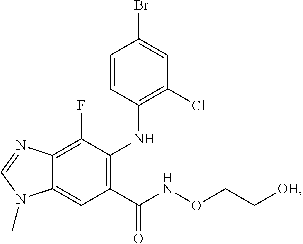 [SMILES: Cn1cnc2c(F)c(Nc3ccc(Br)cc3Cl)c(C(=O)NOCCO)cc21]

as described in Haq, R. et al., WO 2014/138338;

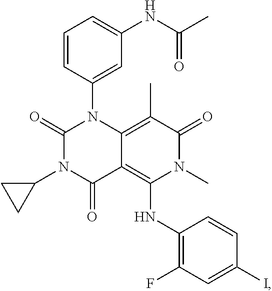 [SMILES: CC(=O)Nc1cccc(-n2c(=O)n(C3CC3)c(=O)c3c(Nc4ccc(I)cc4F)n(C)c(=O)c(C)c32)c1]

as described in Haq, R. et al., WO 2014/138338

In certain embodiments, the EPL is a radical of one of the above compounds, which is attached to L through a modifiable oxygen, nitrogen, or carbon atom.

In certain embodiments, the EPL is a moiety that binds to and inhibits MEK1. In certain embodiments, the EPL is a moiety that binds to and inhibits MEK2. In certain embodiments, the EPL is a moiety that binds to and inhibits both MEK1 and MEK2.

In certain embodiments, the EPL has the formula:

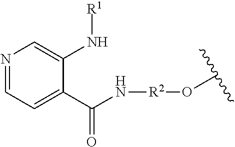

wherein:
R¹ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano; and
R² is —(C₂₋₆ alkylene optionally substituted by one hydroxyl).

In certain embodiments, the EPL is one of the following:

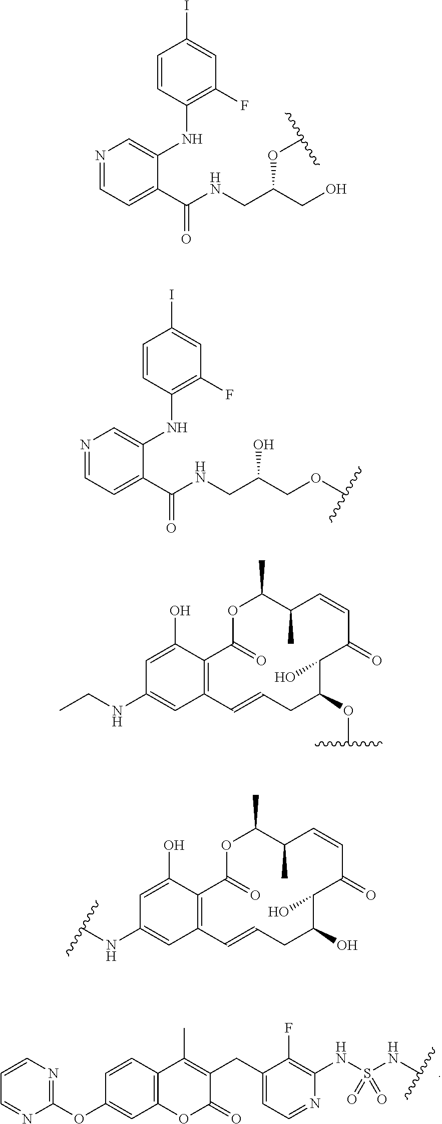 [SMILES: *O[C@H](CO)CNC(=O)c1ccncc1Nc1ccc(I)cc1F ; *OC[C@@H](O)CNC(=O)c1ccncc1Nc1ccc(I)cc1F ; *O[C@H]1C/C=C/c2cc(NCC)cc(O)c2C(=O)O[C@@H](C)[C@H](C)/C=C\C(=O)[C@H]1O ; *Nc1cc(O)c2c(c1)/C=C/C[C@H](O)[C@H](O)C(=O)/C=C\[C@@H](C)[C@H](C)OC2=O ; *NS(=O)(=O)Nc1nccc(Cc2c(C)c3ccc(Oc4ncccn4)cc3oc2=O)c1F]

### J. Moiety for Src

In certain embodiments, the EPL is a moiety that binds to and inhibits proto-oncogene tyrosine-protein kinase (Src). Exemplary compounds that bind to and inhibit Src are reported in the literature, including:

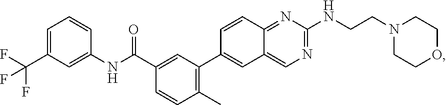 [SMILES: Cc1ccc(C(=O)Nc2cccc(C(F)(F)F)c2)cc1-c1ccc2nc(NCCN3CCOCC3)ncc2c1]

as described in DiMauro, E. et al., in J Med Chem 2006, 49(19): 5671.

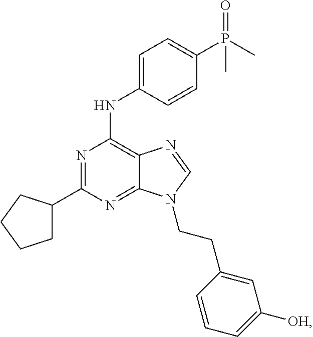 [SMILES: CP(C)(=O)c1ccc(Nc2nc(C3CCCC3)nc3c2ncn3CCc2cccc(O)c2)cc1]

as described in Verones, V. et al., in Eur J Med Chem 2010, 45(12): 5678.

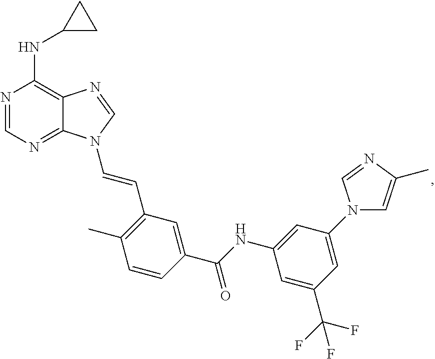 [SMILES: Cc1cn(-c2cc(NC(=O)c3ccc(C)c(/C=C/n4cnc5c(NC6CC6)ncnc54)c3)cc(C(F)(F)F)c2)cn1]

as described in Huang, W. et al., in J Med Chem 2009, 52(15): 4743.

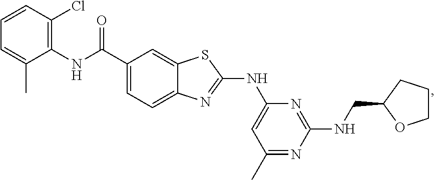 [SMILES: Cc1cc(Nc2nc3ccc(C(=O)Nc4c(C)cccc4Cl)cc3s2)nc(NC[C@H]2CCCO2)n1]

as described in Das, J. et al., in Bioorg Med Chem Lett 2003, 13(15): 2587.

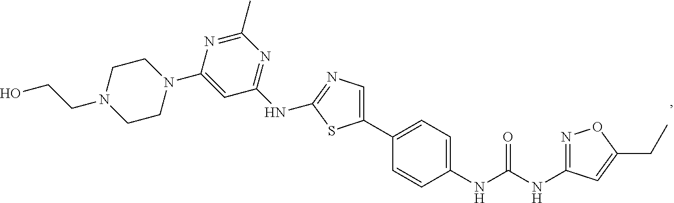 [SMILES: CCc1cc(NC(=O)Nc2ccc(-c3cnc(Nc4cc(N5CCN(CCO)CC5)nc(C)n4)s3)cc2)no1]

as described in Tsai, H. et al., in Cancer Sci 2018, 109(11): 3591.

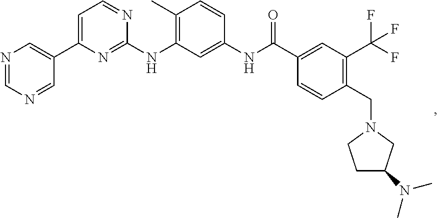 [SMILES: Cc1ccc(NC(=O)c2ccc(CN3CC[C@H](N(C)C)C3)c(C(F)(F)F)c2)cc1Nc1nccc(-c2cncnc2)n1]

as described in Kimura, S. et al., in Blood 2005, 106(11), Abst 1522.

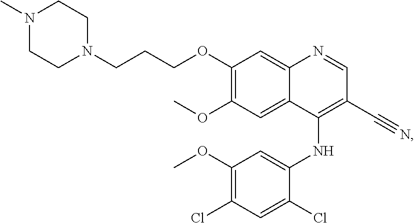 [SMILES: COc1cc(Nc2c(C#N)cnc3cc(OCCCN4CCN(C)CC4)c(OC)cc23)c(Cl)cc1Cl]

as described in Boschelli, D. et al., in Bioorg Med Chem Lett 2003, 13(21): 3797.

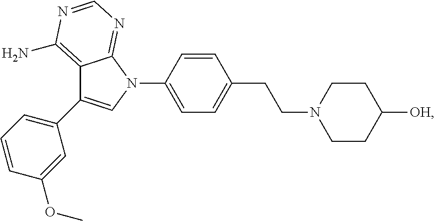 [SMILES: COc1cccc(-c2cn(-c3ccc(CCN4CCC(O)CC4)cc3)c3ncnc(N)c23)c1]

as described in Missbach, M. et al., in Bone 1999, 24(5): 437.

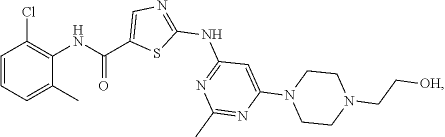 [SMILES: Cc1nc(Nc2ncc(C(=O)Nc3c(C)cccc3Cl)s2)cc(N2CCN(CCO)CC2)n1]

as described in Zhang, C. et al., in J Med Chem 2015, 58(9): 3957.

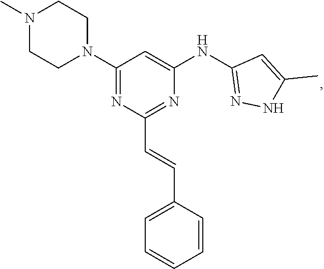

as described in Fletcher, G. et al., in Mol Cancer Ther 2011, 10(1): 126.

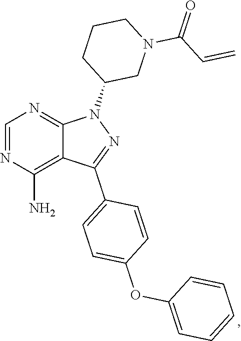 [SMILES: C=CC(=O)N1CCC[C@@H](n2nc(-c3ccc(Oc4ccccc4)cc3)c3c(N)ncnc32)C1]

as described in Crawford, J. et al., in J Med Chem 2018, 61(6): 2227.

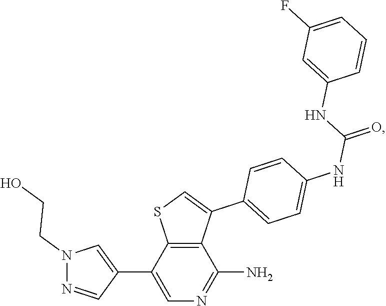 [SMILES: Nc1ncc(-c2cnn(CCO)c2)c2scc(-c3ccc(NC(=O)Nc4cccc(F)c4)cc3)c12]

as described in Glaser, K. et al., in AACR-NCI-EORTC Int Conf Mol Targets Cancer Ther 2011-11-12/2011-11-16-San Francisco, United States Abst A239, Mol Cancer Ther 2011, 10 (Suppl. 1).

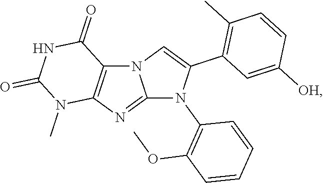 [SMILES: COc1ccccc1-n1c(-c2cc(O)ccc2C)cn2c3c(=O)[nH]c(=O)n(C)c3nc12]

as described in Lafleur, K. et al., in J Med Chem 2013, 56(1): 84.

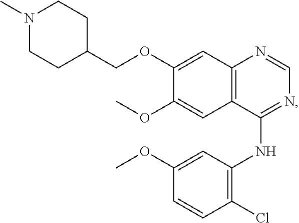 [SMILES: COc1ccc(Cl)c(Nc2ncnc3cc(OCC4CCN(C)CC4)c(OC)cc23)c1]

as described in Ple, P. et al., in J Med Chem 2004, 47(4): 871.

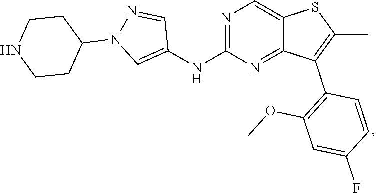 [SMILES: COc1cc(F)ccc1-c1c(C)sc2cnc(Nc3cnn(C4CCNCC4)c3)nc12]

as described in Zhang, N. et al., in WO 2017/012559.

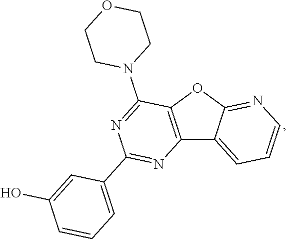 [SMILES: Oc1cccc(-c2nc(N3CCOCC3)c3oc4ncccc4c3n2)c1]

as described in Zhong, Y. et al., in Bioorg Med Chem 2013, 21(7): 1724.

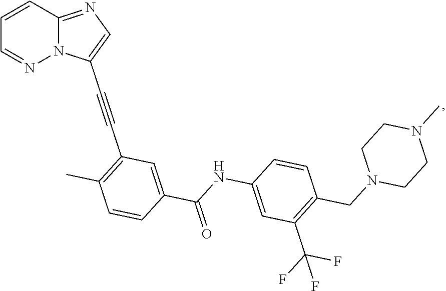 [SMILES: Cc1ccc(C(=O)Nc2ccc(CN3CCN(C)CC3)c(C(F)(F)F)c2)cc1C#Cc1cnc2cccnn12]

as described in Zhang, C. et al., in J Med Chem 2015, 58(9): 3957.

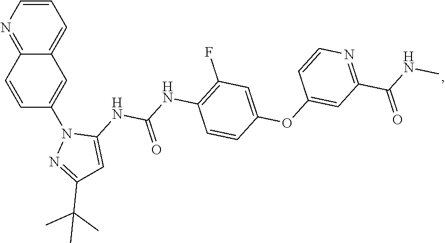 [SMILES: CNC(=O)c1cc(Oc2ccc(NC(=O)Nc3cc(C(C)(C)C)nn3-c3ccc4ncccc4c3)c(F)c2)ccn1]

as described in Wise, C. et al., in Proc Am Assoc Cancer Res (AACR) 2008, 49, Abst 4869.

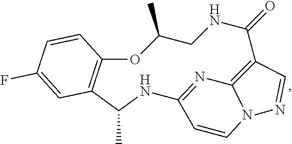 [SMILES: C[C@H]1CNC(=O)c2cnn3ccc(nc23)N[C@H](C)c2cc(F)ccc2O1]

as described in Drilon, A. et al., in Cancer Discov 2018, 8(10): 1227.

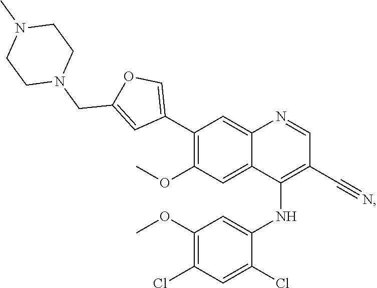 [SMILES: COc1cc(Nc2c(C#N)cnc3cc(-c4coc(CN5CCN(C)CC5)c4)c(OC)cc23)c(Cl)cc1Cl]

as described in Boschelli, D. et al., in Proc Am Assoc Cancer Res (AACR) 2006, 47, Abst 4751.

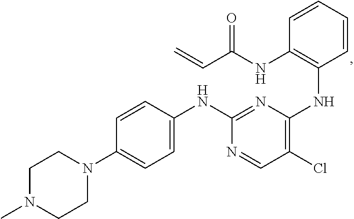 [SMILES: C=CC(=O)Nc1ccccc1Nc1nc(Nc2ccc(N3CCN(C)CC3)cc2)ncc1Cl]

as described in Du, G. et al., in J Med Chem 2020, 63(4): 1624.

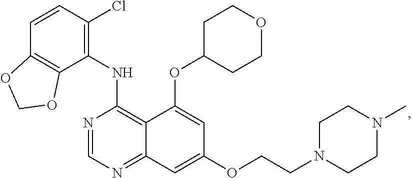 [SMILES: CN1CCN(CCOc2cc(OC3CCOCC3)c3c(Nc4c(Cl)ccc5c4OCO5)ncnc3c2)CC1]

as described in Hiscox, S. et al., in Eur J Cancer Suppl 2004, 2(8), Abst 406.

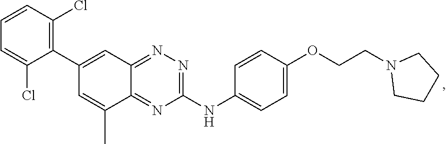 [SMILES: Cc1cc(-c2c(Cl)cccc2Cl)cc2nnc(Nc3ccc(OCCN4CCCC4)cc3)nc12]

as described in Noronha, G. et al., in Bioorg Med Chem Lett 2007, 17(3): 602.

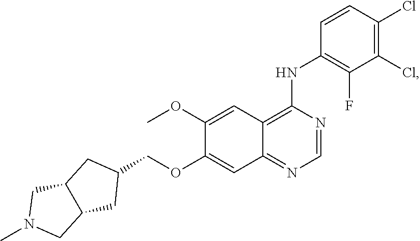

as described in Frost, P. et al., in WO 2012/027537.

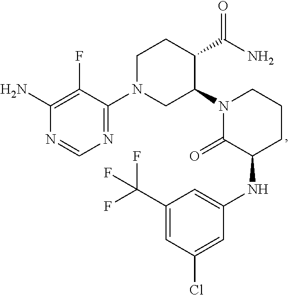 [SMILES: NC(=O)[C@H]1CCN(c2ncnc(N)c2F)C[C@@H]1N1CCC[C@@H](Nc2cc(Cl)cc(C(F)(F)F)c2)C1=O]

as described in Fabian, C. et al., in 108th Annu Meet Am Assoc Cancer Res (AACR) 2017-04-01/2017-04-05 Washington, D.C., United States-Abst 1207, Cancer Res 2017, 77(13).

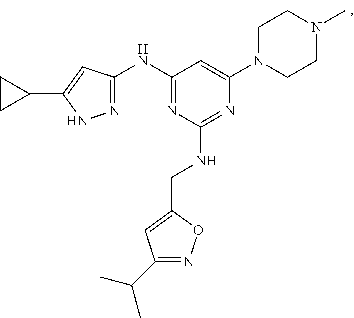 [SMILES: CC(C)c1cc(CNc2nc(Nc3cc(C4CC4)[nH]n3)cc(N3CCN(C)CC3)n2)on1]

as described in Britten, C. et al., in Eur J Cancer Suppl 2008, 6(12), Abst 390.

In certain embodiments, the EPL is a radical of one of the above compounds, which is attached to L through a modifiable oxygen, nitrogen, or carbon atom.

In certain embodiments, the EPL is one of the following:

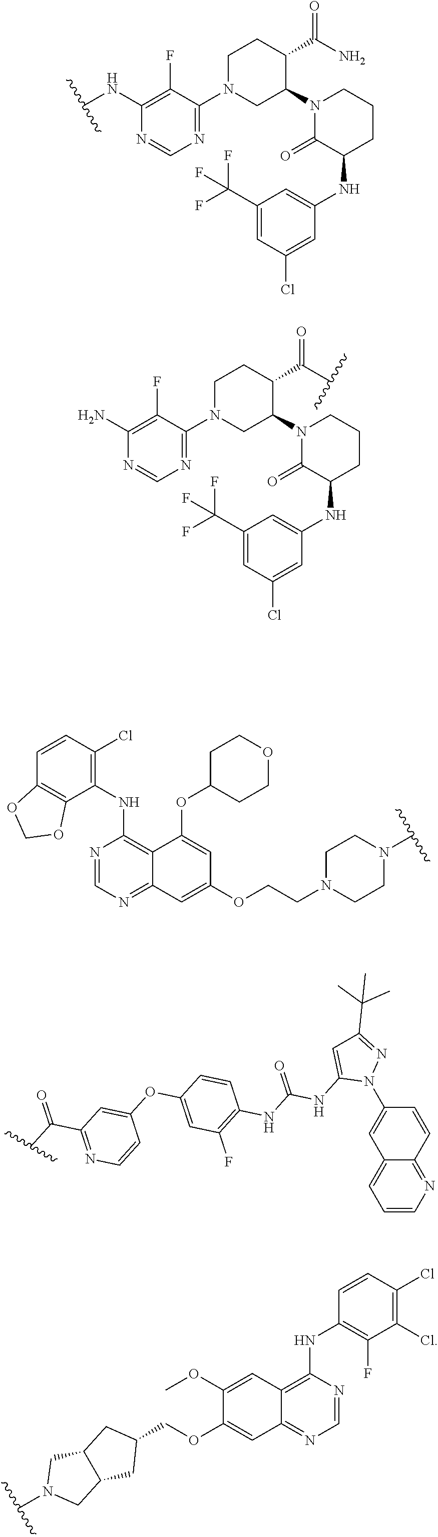

K Moiety for c-KIT

In certain embodiments, the EPL is a moiety that binds to c-KIT. Exemplary compounds that bind to c-KIT are reported in the literature, including:

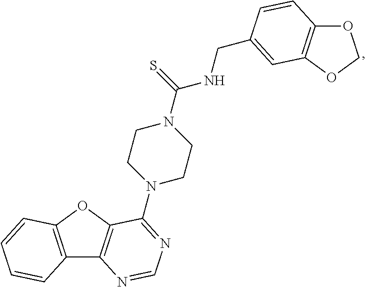 [SMILES: S=C(NCc1ccc2c(c1)OCO2)N1CCN(c2ncnc3c2oc2ccccc23)CC1]

as described by Mahadevan, D. et al., in Oncogene 2007, vol 26(27), page.

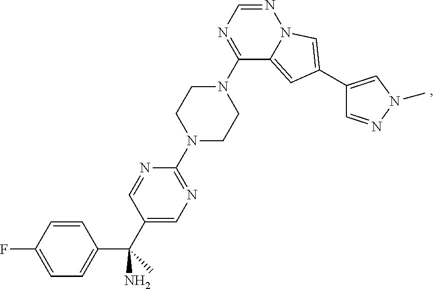 [SMILES: Cn1cc(-c2cc3c(N4CCN(c5ncc([C@@](C)(N)c6ccc(F)cc6)cn5)CC4)ncnn3c2)cn1]

as described by An, Q. et al., in WO2019034128.

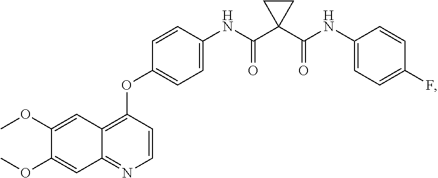 [SMILES: COc1cc2nccc(Oc3ccc(NC(=O)C4(C(=O)Nc5ccc(F)cc5)CC4)cc3)c2cc1OC]

as described by Zhao, Y. et al., in Bioorg. Med. Chem. 2017, vol 25(12), page 3195.

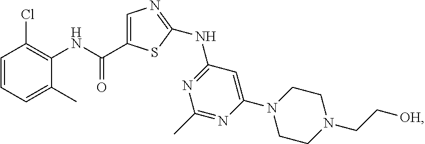 [SMILES: Cc1nc(Nc2ncc(C(=O)Nc3c(C)cccc3Cl)s2)cc(N2CCN(CCO)CC2)n1]

as described by Yao, G. et al., in CN106749223.

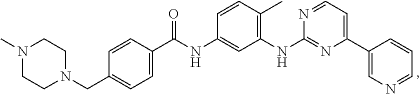 [SMILES: Cc1ccc(NC(=O)c2ccc(CN3CCN(C)CC3)cc2)cc1Nc1nccc(-c2cccnc2)n1]

as described by Wang, J. et al., in CN102675289.

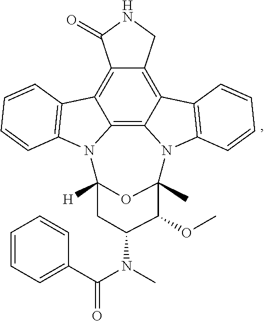 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4]

as described by Jain, V. K. et al., in US2019183880.

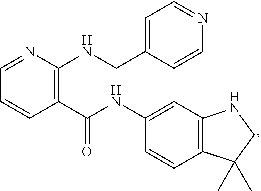 [SMILES: CC1(C)CNc2cc(NC(=O)c3cccnc3NCc3ccncc3)ccc21]

as described by Jain, V. K. et al., in US2019183880.

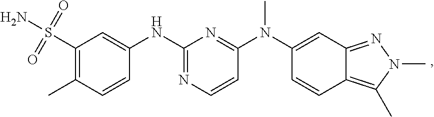 [SMILES: Cc1ccc(Nc2nccc(N(C)c3ccc4c(C)n(C)nc4c3)n2)cc1S(N)(=O)=O]

as described by Min, K. H. et al., in KR2016147170.

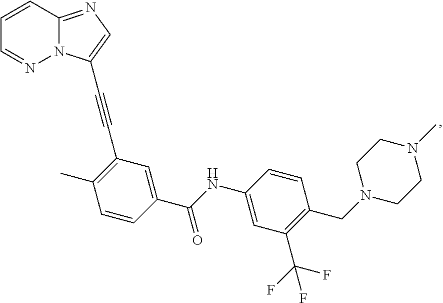 [SMILES: Cc1ccc(C(=O)Nc2ccc(CN3CCN(C)CC3)c(C(F)(F)F)c2)cc1C#Cc1cnc2cccnn12]

as described by Huang, W.-S. et al., in WO2011053938.

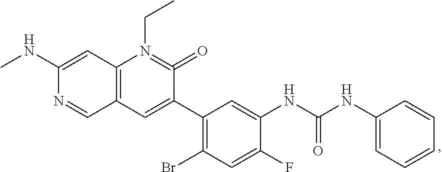

as described by Flynn, D. L. et al., in U.S. Pat. No. 8,461,179.

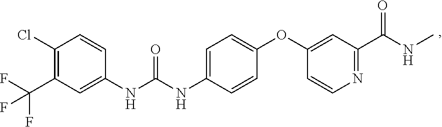 [SMILES: CNC(=O)c1cc(Oc2ccc(NC(=O)Nc3ccc(Cl)c(C(F)(F)F)c3)cc2)ccn1]

as described by Murphy, E. A. et al., in WO2011097594.

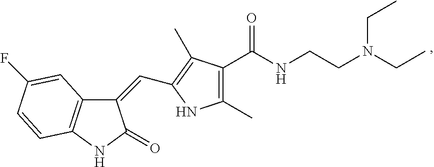 [SMILES: CCN(CC)CCNC(=O)c1c(C)[nH]c(/C=C2\C(=O)Nc3ccc(F)cc32)c1C]

as described by Flynn, D. L. et al., in U.S. Pat. No. 8,461,179.

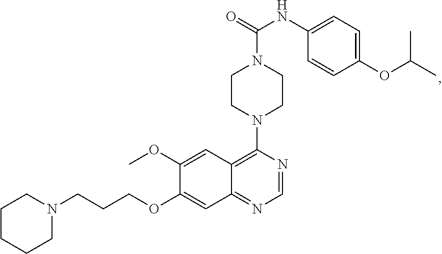 [SMILES: COc1cc2c(N3CCN(C(=O)Nc4ccc(OC(C)C)cc4)CC3)ncnc2cc1OCCCN1CCCCC1]

as described by Russu, W. A. et al., in WO2012027495.

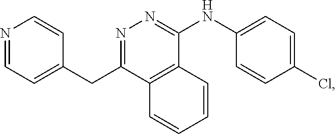 [SMILES: Clc1ccc(Nc2nnc(Cc3ccncc3)c3ccccc23)cc1]

as described by Sun, P. et al., in WO2010031266

In certain embodiments, the EPL is a radical of one of the above compounds, which is attached to L through a modifiable oxygen, nitrogen, or carbon atom.

In certain embodiments, the EPL is a radical of dasatinib, imatinib mesylate (STI571), sunitinib, regorafenib (BAY 73-4506), pazopanib HCl (GW786034 HCl), dovitinib (TKI-258), masitinib (AB1010), tivozanib (AV-951), motesanib diphosphate (AMG-706), amuvatinib (MP-470), levatinib (E7080), osi-930, Ki8751, telatinib, pozopanib, dovitinib (TKI-258), ripretinib (DCC-2618), sunitinib, Ki20227, avapritinib (BLU-285), AZD3229, AZD2932, regorafenib monohydrate, dovitinib (TKI258), pexidartinib (PLX3397), PDGFR inhibitor 1, or sitravatinib (MGCD516).

In certain embodiments, the EPL is one of the following:

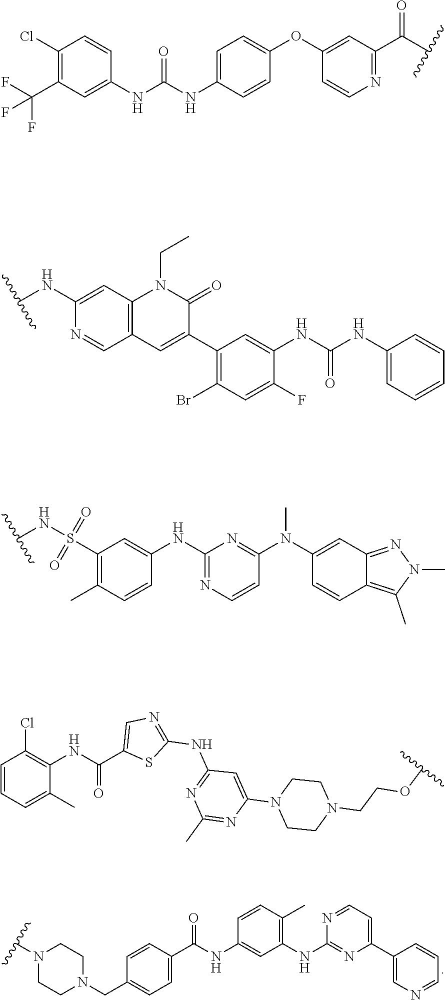 [SMILES: *C(=O)c1cc(Oc2ccc(NC(=O)Nc3ccc(Cl)c(C(F)(F)F)c3)cc2)ccn1 ; *Nc1cc2c(cn1)cc(-c1cc(NC(=O)Nc3ccccc3)c(F)cc1Br)c(=O)n2CC ; *NS(=O)(=O)c1cc(Nc2nccc(N(C)c3ccc4c(C)n(C)nc4c3)n2)ccc1C ; *OCCN1CCN(c2cc(Nc3ncc(C(=O)Nc4c(C)cccc4Cl)s3)nc(C)n2)CC1 ; *N1CCN(Cc2ccc(C(=O)Nc3ccc(C)c(Nc4nccc(-c5cccnc5)n4)c3)cc2)CC1]

### L. Moiety for KIF11

In certain embodiments, the EPL is a moiety that binds to Kinesin Family Member 11 (KIF11). Exemplary compounds that bind to KIFI1 are reported in the literature, including:

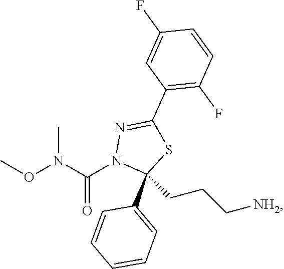 [SMILES: CON(C)C(=O)N1N=C(c2cc(F)ccc2F)S[C@@]1(CCCN)c1ccccc1]

as described by Hans, J., et al. in WO2006/44825;

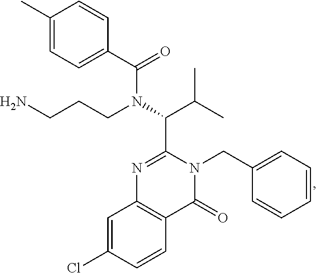 [SMILES: Cc1ccc(C(=O)N(CCCN)[C@@H](c2nc3cc(Cl)ccc3c(=O)n2Cc2ccccc2)C(C)C)cc1]

as described by Bergnes, G., et al. in WO2003/070701;

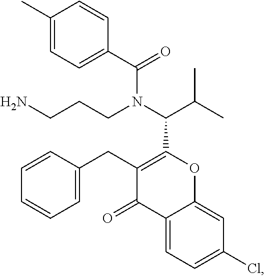 [SMILES: Cc1ccc(C(=O)N(CCCN)[C@@H](c2oc3cc(Cl)ccc3c(=O)c2Cc2ccccc2)C(C)C)cc1]

as described by Wood, K. W., et al. in WO2007/067752;

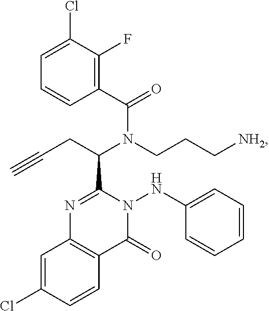 [SMILES: C#CC[C@H](c1nc2cc(Cl)ccc2c(=O)n1Nc1ccccc1)N(CCCN)C(=O)c1cccc(Cl)c1F]

as described by Liu, J., et al. in WO2009/002808;

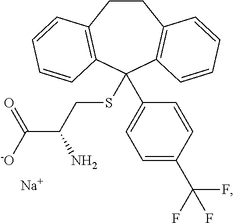

as described by Liu, J., et al. in WO2013/141264

In certain embodiments, the EPL is a radical of one of the above compounds, which is attached to L through a modifiable oxygen, nitrogen, or carbon atom.

In certain embodiments, the EPL has the following formula:

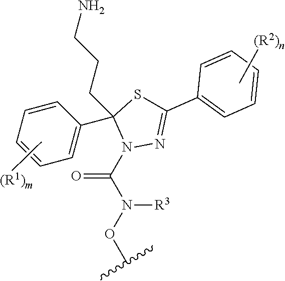

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ is H, C₁-C₆ alkyl, C₁-C₆ haloalkyl, or C₃-C₆ cycloalkyl; and m and n each represent independently 0, 1, 2, or 3.

In certain embodiments, the EPL has the following formula:

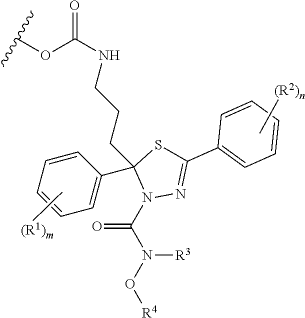

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ and R⁴ each represent independently H, C₁-C₆ alkyl, C₁-C₆ haloalkyl, or C₃-C₆ cycloalkyl; and
m and n each represent independently 0, 1, 2, or 3.

In certain embodiments, the EPL has the following formula:

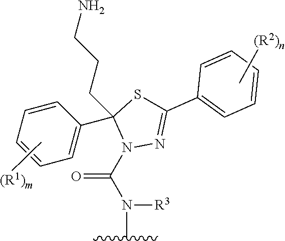

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ is H, C₁-C₆ alkyl, C₁-C₆ haloalkyl, or C₃-C₆ cycloalkyl; and
m and n each represent independently 0, 1, 2, or 3.

In certain embodiments, the EPL is one of the following:

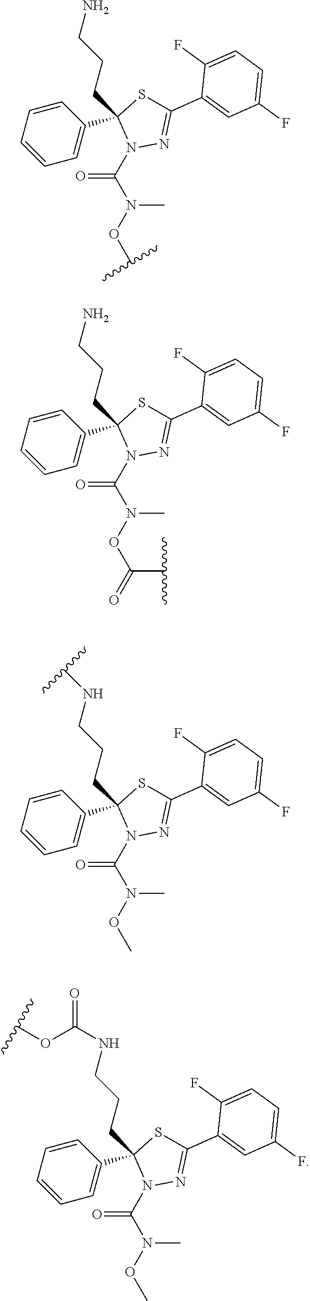 [SMILES: *ON(C)C(=O)N1N=C(c2cc(F)ccc2F)S[C@@]1(CCCN)c1ccccc1 ; *C(=O)ON(C)C(=O)N1N=C(c2cc(F)ccc2F)S[C@@]1(CCCN)c1ccccc1 ; *NCCC[C@@]1(c2ccccc2)SC(c2cc(F)ccc2F)=NN1C(=O)N(C)OC ; *OC(=O)NCCC[C@@]1(c2ccccc2)SC(c2cc(F)ccc2F)=NN1C(=O)N(C)OC]

In certain embodiments, the EPL is

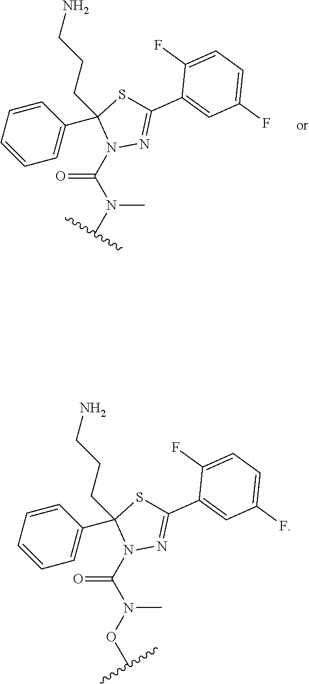 [SMILES: *N(C)C(=O)N1N=C(c2cc(F)ccc2F)SC1(CCCN)c1ccccc1 ; *ON(C)C(=O)N1N=C(c2cc(F)ccc2F)SC1(CCCN)c1ccccc1]

In certain embodiments, the EPL is one of the following:

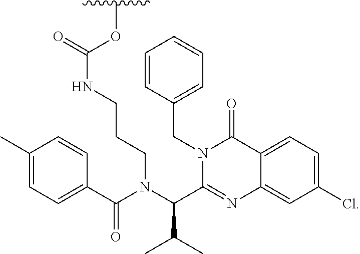 [SMILES: *OC(=O)NCCCN(C(=O)c1ccc(C)cc1)[C@@H](c1nc2cc(Cl)ccc2c(=O)n1Cc1ccccc1)C(C)C]

### M. Moiety for HSP90

In certain embodiments, the EPL is a moiety that binds to HSP90. Exemplary compounds that bind to HSP90 are reported in the literature, including:

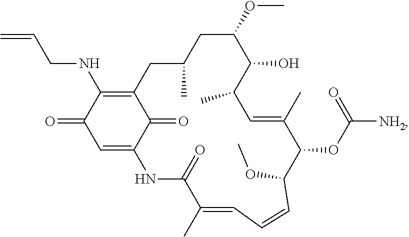 [SMILES: C=CCNC1=C2C[C@@H](C)C[C@H](OC)[C@H](O)[C@@H](C)/C=C(\C)[C@H](OC(N)=O)[C@@H](OC)/C=C\C=C(/C)C(=O)NC(=CC1=O)C2=O]

as described by Gallaschun, R. J., et al. in WO1995/001342;

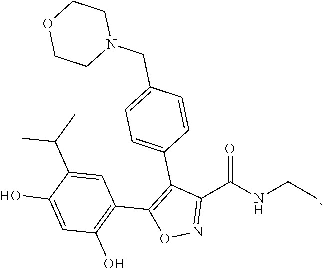 [SMILES: CCNC(=O)c1noc(-c2cc(C(C)C)c(O)cc2O)c1-c1ccc(CN2CCOCC2)cc1]

as described by Finch, H., et al. in WO2004/072051;

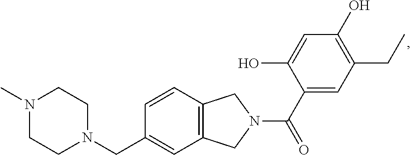 [SMILES: CCc1cc(C(=O)N2Cc3ccc(CN4CCN(C)CC4)cc3C2)c(O)cc1O]

as described by Chessari, G., et al. in WO2006/109085;

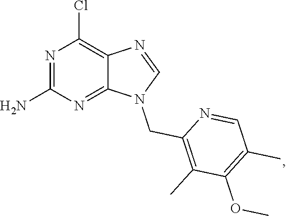 [SMILES: COc1c(C)cnc(Cn2cnc3c(Cl)nc(N)nc32)c1C]

as described by Kasibhatla, S. R., et al. in WO2005/028434;

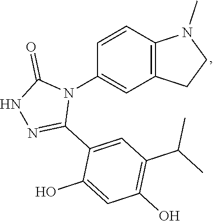 [SMILES: CC(C)c1cc(-c2n[nH]c(=O)n2-c2ccc3c(c2)CCN3C)c(O)cc1O]

as described by Ying, W., et al. in WO2006/055760;

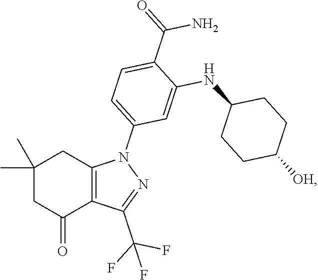 [SMILES: CC1(C)CC(=O)c2c(C(F)(F)F)nn(-c3ccc(C(N)=O)c(N[C@H]4CC[C@H](O)CC4)c3)c2C1]

as described by Huang, K. H., et al. in WO2006/091963;

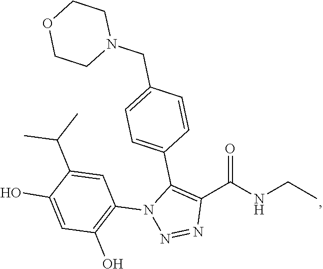 [SMILES: CCNC(=O)c1nnn(-c2cc(C(C)C)c(O)cc2O)c1-c1ccc(CN2CCOCC2)cc1]

as described by Giannini, G., et al. in WO2012/084602.

In certain embodiments, the EPL is a radical of one of the above compounds, which is attached to L through a modifiable oxygen, nitrogen, or carbon atom.

In certain embodiments, the EPL is one of the following:

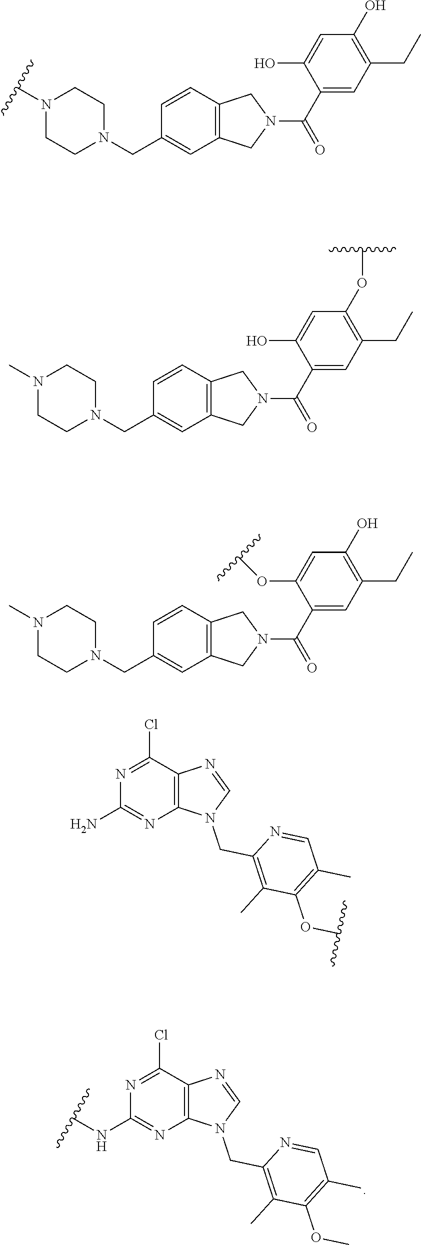

### N. Moiety for Tubulin

In certain embodiments, the EPL is a moiety that binds to tubulin. Exemplary compounds that bind to tubulin are reported in the literature, including:

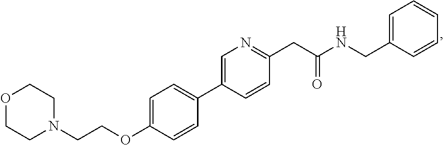 [SMILES: O=C(Cc1ccc(-c2ccc(OCCN3CCOCC3)cc2)cn1)NCc1ccccc1]

as described by Hangauer, D. G., et at. in WO2006/071960;

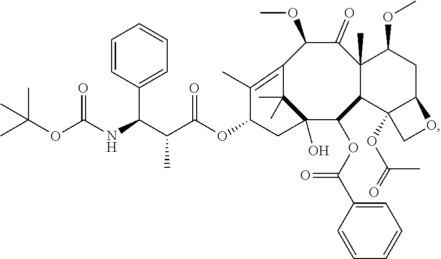

as described by Bouchard, H., et al. in WO1996/030355;

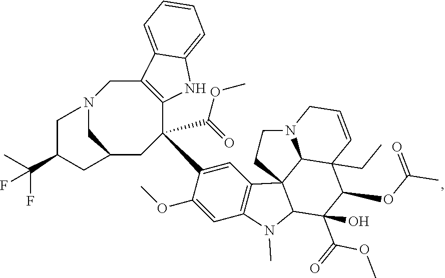

as described by Duflos, A., et at. in WO1998/045301;

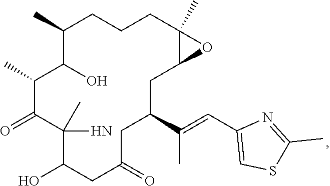

as described by Vite, G. D., et at. in WO1999/002514;

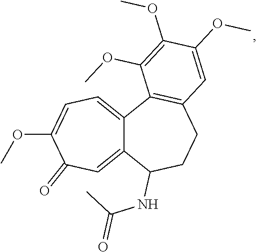

as described by Brown, D. M., et al. in WO2002/056872;

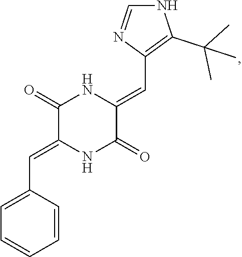 [SMILES: CC(C)(C)c1[nH]cnc1/C=c1\[nH]c(=O)/c(=C/c2ccccc2)[nH]c1=O]

as described by Hayashi, Y., et al. in WO2004/054498;

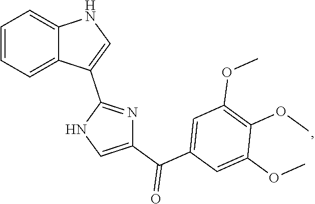 [SMILES: COc1cc(C(=O)c2c[nH]c(-c3c[nH]c4ccccc34)n2)cc(OC)c1OC]

as described by Dalton, J. T., et al. in WO2012/027481;

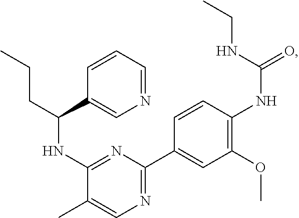 [SMILES: CCC[C@H](Nc1nc(-c2ccc(NC(=O)NCC)c(OC)c2)ncc1C)c1cccnc1]

as described by Burns, C. J., et al. in WO2005/054199;

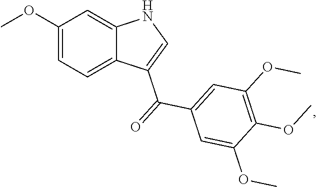

as described by Hsieh, H.-P., et al. in US2003/195244;

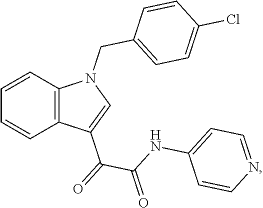 [SMILES: O=C(Nc1ccncc1)C(=O)c1cn(Cc2ccc(Cl)cc2)c2ccccc12]

as described by Lebaut, G., et al. in WO1998/009946;

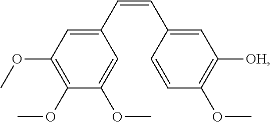 [SMILES: COc1ccc(/C=C\c2cc(OC)c(OC)c(OC)c2)cc1O]

as described by Lorenz, G., et al. in US2005/065595.

In certain embodiments, the EPL is a radical of one of the above compounds, which is attached to L through a modifiable oxygen, nitrogen, or carbon atom.

In certain embodiments, the EPL is one of the following:

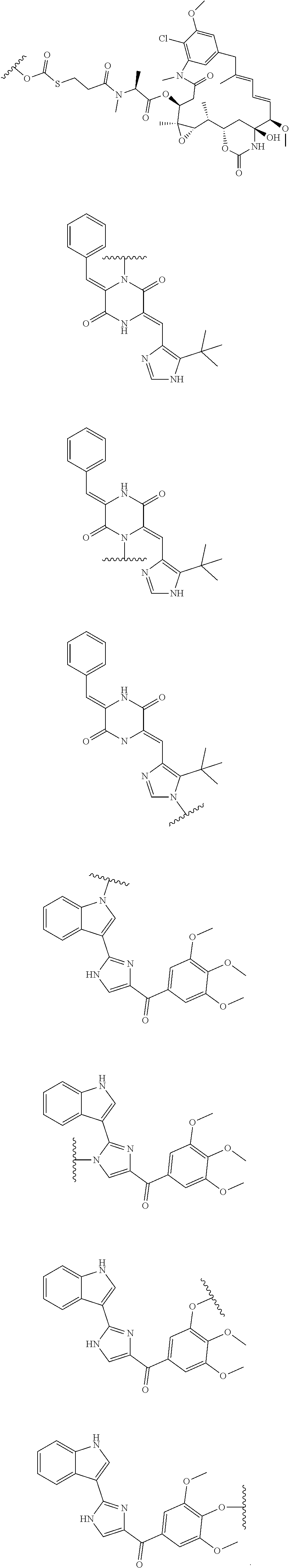

In certain embodiments, the EPL is one of the following:

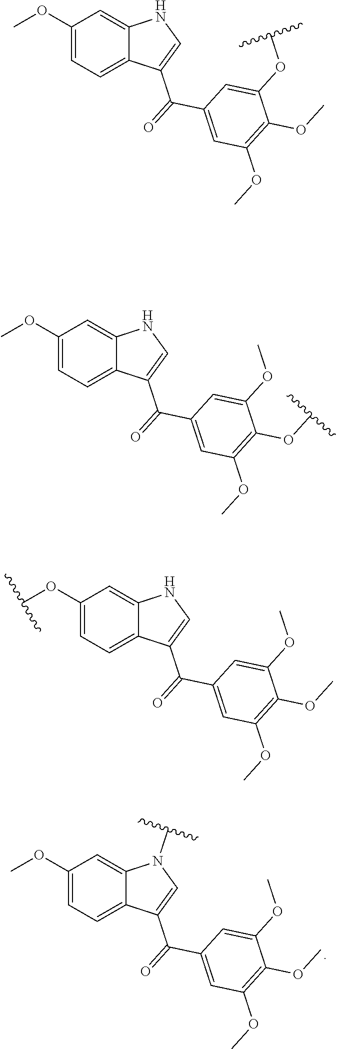

### O. Moiety for Proteasome

In certain embodiments, the EPL is a moiety that binds to and/or inhibits the proteasome. Exemplary compounds that bind to and/or inhibit the proteasome are reported in the literature, including:

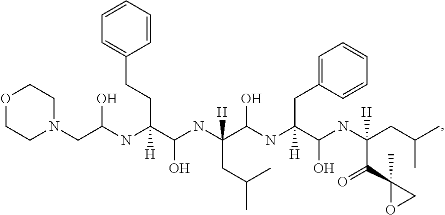 [SMILES: CC(C)C[C@H](NC(O)[C@H](Cc1ccccc1)NC(O)[C@H](CC(C)C)NC(O)[C@H](CCc1ccccc1)NC(O)CN1CCOCC1)C(=O)[C@@]1(C)CO1]

as described by Smyth, M. S., et al. in WO2005/105827;

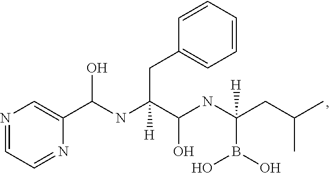 [SMILES: CC(C)C[C@H](NC(O)[C@H](Cc1ccccc1)NC(O)c1cnccn1)B(O)O]

as described by Adams, J., et al. in WO1996/013266;

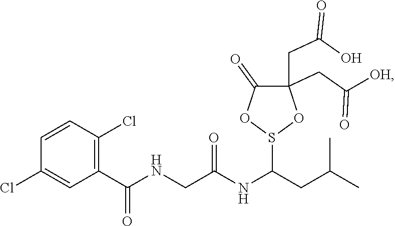

as described by Zhan, F., et al. in WO2010/151731;

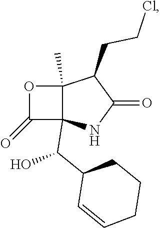 [SMILES: C[C@@]12OC(=O)[C@]1([C@@H](O)[C@@H]1C=CCCC1)NC(=O)[C@@H]2CCCl]

as described by Fenical, W., et al. in WO2002/047610;

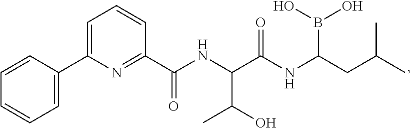 [SMILES: CC(C)CC(NC(=O)C(NC(=O)c1cccc(-c2ccccc2)n1)C(C)O)B(O)O]

as described by Chatterjee, S., et al. in WO2005/021558;

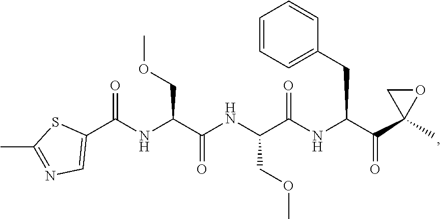

as described by Zhou, H.-J., et al. in US2007/105786;

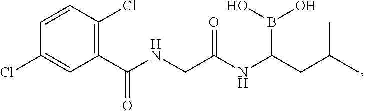 [SMILES: CC(C)CC(NC(=O)CNC(=O)c1cc(Cl)ccc1Cl)B(O)O]

as described by Olhava, E. J., et al. in WO2009/020448;

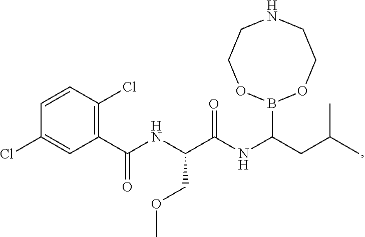 [SMILES: COC[C@H](NC(=O)c1cc(Cl)ccc1Cl)C(=O)NC(CC(C)C)B1OCCNCCO1]

as described by Qin, Y., et al. in WO2019/228299.

In certain embodiments, the EPL is a radical of one of the above compounds, which is attached to L through a modifiable oxygen, nitrogen, or carbon atom.

In certain embodiments, the EPL is one of the following:

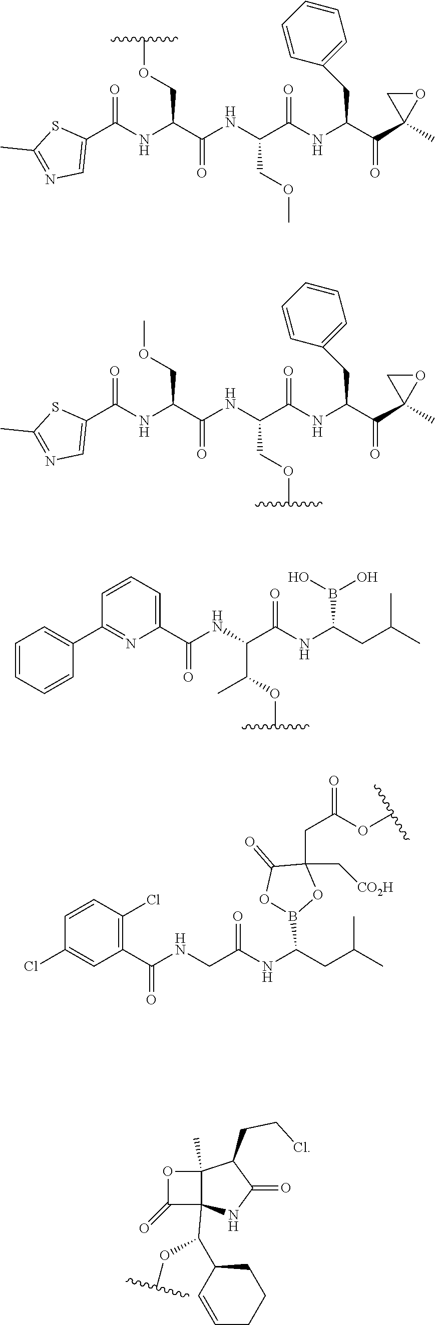

### P. Moiety for Topoisomerase

In certain embodiments, the EPL is a moiety that binds to topoisomerase.

In certain embodiments, the EPL is a moiety that binds to DNA Topoisomerase I (TOP1). Exemplary compounds that bind to TOP1 are reported in the literature, including:

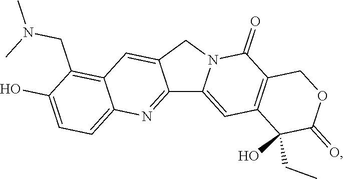 [SMILES: CC[C@@]1(O)C(=O)OCc2c1cc1n(c2=O)Cc2cc3c(CN(C)C)c(O)ccc3nc2-1]

as described by Johnson, R. K., et al. in WO1992/014471;

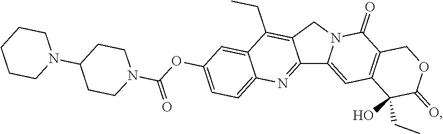 [SMILES: CCc1c2c(nc3ccc(OC(=O)N4CCC(N5CCCCC5)CC4)cc13)-c1cc3c(c(=O)n1C2)COC(=O)[C@]3(O)CC]

as described by Caruso, M., et al. in WO1999/048503;

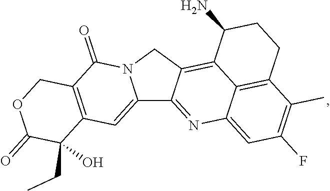 [SMILES: CC[C@@]1(O)C(=O)OCc2c1cc1n(c2=O)Cc2c-1nc1cc(F)c(C)c3c1c2[C@@H](N)CC3]

as described by Terasawa, H., et al. in EP0495432;

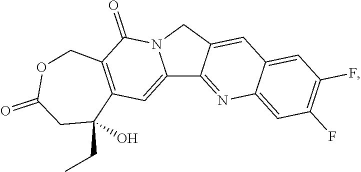 [SMILES: CC[C@@]1(O)CC(=O)OCc2c1cc1n(c2=O)Cc2cc3cc(F)c(F)cc3nc2-1]

as described by Bigg, D., et al. in WO1999/011646;

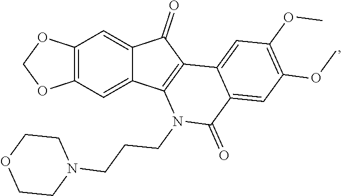 [SMILES: COc1cc2c3c(n(CCCN4CCOCC4)c(=O)c2cc1OC)-c1cc2c(cc1C3=O)OCO2]

as described by Cushman, M. S., et al. in WO2007/059008;

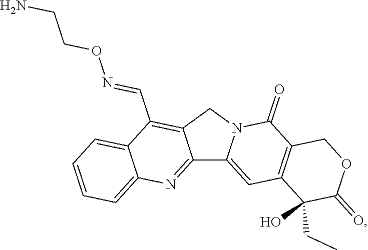 [SMILES: CC[C@@]1(O)C(=O)OCc2c1cc1n(c2=O)Cc2c-1nc1ccccc1c2/C=N/OCCN]

as described by Penco, S., et al. in WO2000/053607;

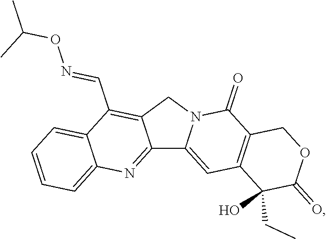 [SMILES: CC[C@@]1(O)C(=O)OCc2c1cc1n(c2=O)Cc2c-1nc1ccccc1c2/C=N/OC(C)C]

as described by Penco. S, et al. in WO2000/053607;

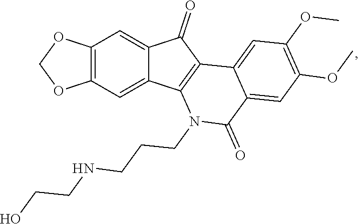 [SMILES: COc1cc2c3c(n(CCCNCCO)c(=O)c2cc1OC)-c1cc2c(cc1C3=O)OCO2]

as described by Cushman, M., et al. in J Med Chem 2000, 43(20): 3688;

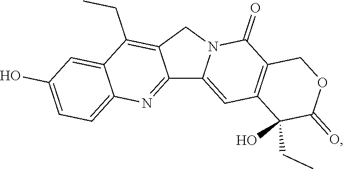 [SMILES: CCc1c2c(nc3ccc(O)cc13)-c1cc3c(c(=O)n1C2)COC(=O)[C@]3(O)CC]

as described by Rahman, A., et al. in WO2002/058622.

In certain embodiments, the EPL is a radical of one of the above compounds, which is attached to L through a modifiable oxygen, nitrogen, or carbon atom.

In certain embodiments, the EPL is is a moiety that bind to topoisomerase I (TOP1).

In certain embodiments, the EPL is one of the following:

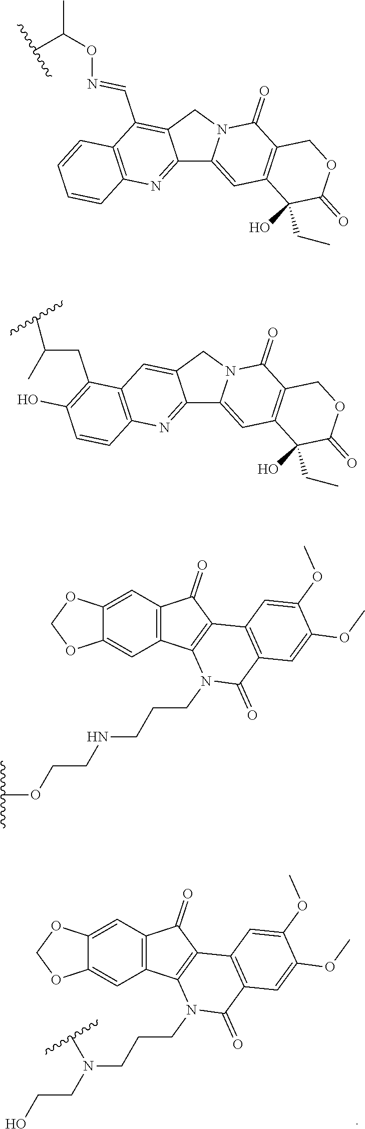 [SMILES: *C(C)O/N=C/c1c2c(nc3ccccc13)-c1cc3c(c(=O)n1C2)COC(=O)[C@]3(O)CC ; *C(C)Cc1c(O)ccc2nc3c(cc12)Cn1c-3cc2c(c1=O)COC(=O)[C@]2(O)CC ; *OCCNCCCn1c2c(c3cc(OC)c(OC)cc3c1=O)C(=O)c1cc3c(cc1-2)OCO3 ; *N(CCO)CCCn1c2c(c3cc(OC)c(OC)cc3c1=O)C(=O)c1cc3c(cc1-2)OCO3]

In certain embodiments, the EPL is the following:

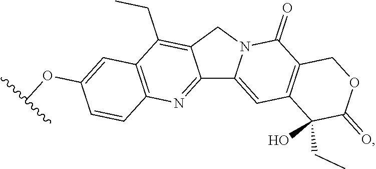 [SMILES: *Oc1ccc2nc3c(c(CC)c2c1)Cn1c-3cc2c(c1=O)COC(=O)[C@]2(O)CC]

### O. Moiety for HDAC

In certain embodiments, the EPL is a moiety that binds to Histone Deacetylase (HDAC). Exemplary compounds that bind to HDAC are reported in the literature, including:

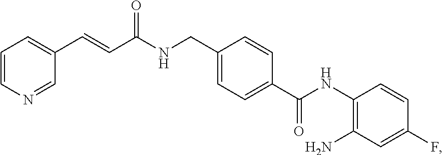 [SMILES: Nc1cc(F)ccc1NC(=O)c1ccc(CNC(=O)/C=C/c2cccnc2)cc1]

as described by Lu, X.-P., et al. in WO2004/071400;

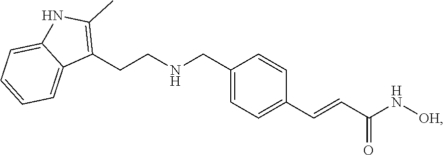 [SMILES: Cc1[nH]c2ccccc2c1CCNCc1ccc(/C=C/C(=O)NO)cc1]

as described by Bair, K. W., et al. in WO2002/022577;

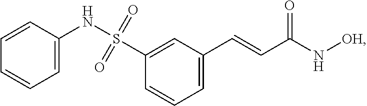 [SMILES: O=C(/C=C/c1cccc(S(=O)(=O)Nc2ccccc2)c1)NO]

as described by Harris, C. J., et al. in WO2002/030879;

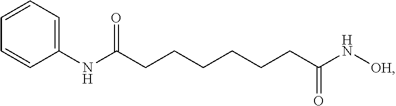 [SMILES: O=C(CCCCCCC(=O)Nc1ccccc1)NO]

as described by Breslow, R., et al. in WO1993/007148;

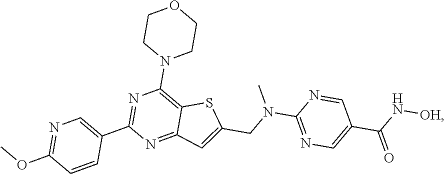 [SMILES: COc1ccc(-c2nc(N3CCOCC3)c3sc(CN(C)c4ncc(C(=O)NO)cn4)cc3n2)cn1]

as described by Cai, X., et al. in WO2012/135571;

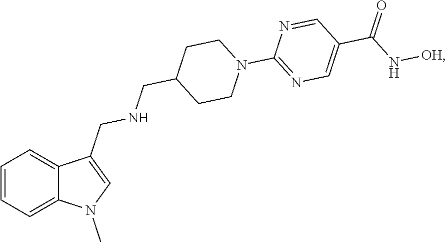 [SMILES: Cn1cc(CNCC2CCN(c3ncc(C(=O)NO)cn3)CC2)c2ccccc21]

as described by Verdonck, M. G. C., et al. in WO2006/010750;

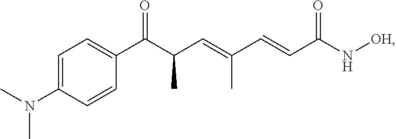 [SMILES: CC(/C=C/C(=O)NO)=C\[C@@H](C)C(=O)c1ccc(N(C)C)cc1]

as described by Shibai, H., et al. in JP1985/149520;

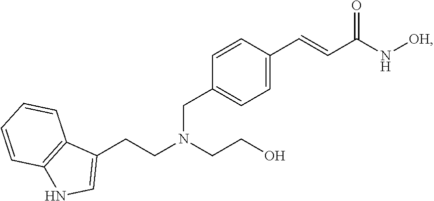 [SMILES: O=C(/C=C/c1ccc(CN(CCO)CCc2c[nH]c3ccccc23)cc1)NO]

as described by Bair, K. W., et al. in WO2002/022577;

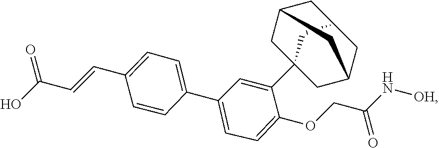 [SMILES: O=C(O)/C=C/c1ccc(-c2ccc(OCC(=O)NO)c([C@]34C[C@H]5C[C@H](C[C@H](C5)C3)C4)c2)cc1]

as described by Pisano, C., et al. in WO2018/060354.

In certain embodiments, the EPL is a radical of one of the above compounds, which is attached to L through a modifiable oxygen, nitrogen, or carbon atom.

In certain embodiments, the EPL is one of the following:

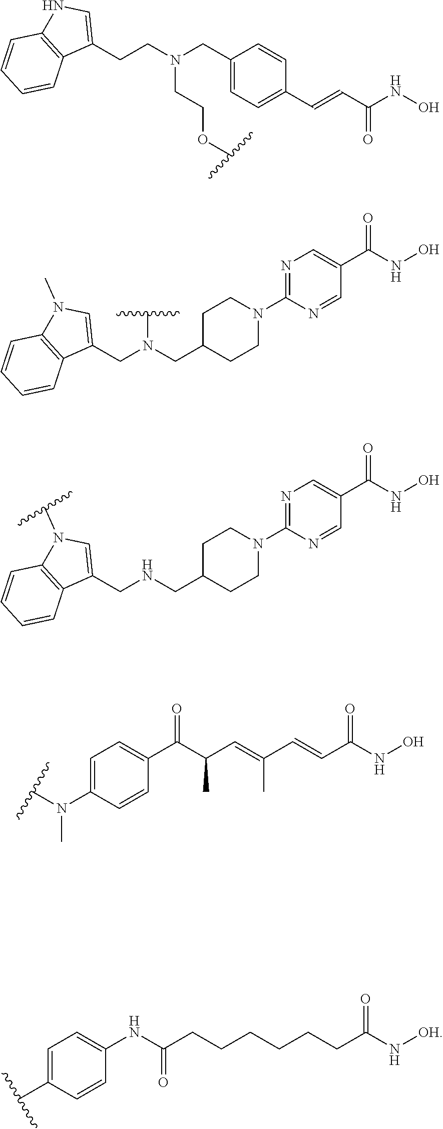 [SMILES: *OCCN(CCc1c[nH]c2ccccc12)Cc1ccc(/C=C/C(=O)NO)cc1 ; *N(Cc1cn(C)c2ccccc12)CC1CCN(c2ncc(C(=O)NO)cn2)CC1 ; *n1cc(CNCC2CCN(c3ncc(C(=O)NO)cn3)CC2)c2ccccc21 ; *N(C)c1ccc(C(=O)[C@H](C)/C=C(C)/C=C/C(=O)NO)cc1 ; *c1ccc(NC(=O)CCCCCCC(=O)NO)cc1]

### Part B: Exemplary Further Description of TPL Component of Compounds of Formula I

Compounds of Formula I may be further characterized according to, for example, the identity of the TPL component. As generally described above, the TPL is a moiety that binds to a target protein selected from KRAS, HER2, or EGFR. In certain embodiments, TPL is a moiety that binds KRAS. In certain embodiments, TPL is a moiety that binds HER2. In certain embodiments, TPL is a moiety that binds EGFR.

Exemplariy moieties for the TPL component are described in more detail below.

### Moiety for HER2

In certain embodiments, the TPL is a moiety that binds to HER2. Exemplary compounds that bind to HER2 are reported in the literature. A radical of such compounds reported in the literature that bind HER2 are amenable for use in the present invention.

In certain embodiments, the TPL is one of the following:

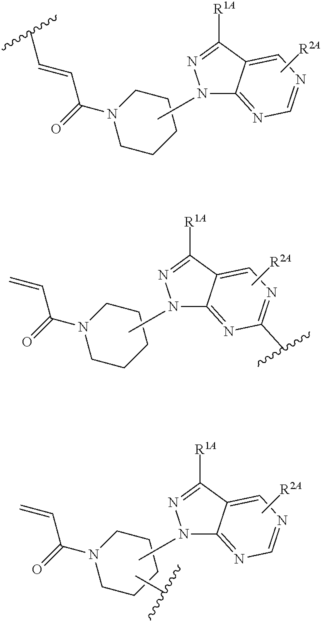

wherein:
R^{1A} is —C(O)(NRS^{A})-(phenyl optionally substituted with 1, 2, 3, or 5 substituents independently selected from halo, hydroxyl, C₁₋₄ alkyl, C₁₋₄ alkoxyl, and —(C₁₋₄ alkylene-C(O)N(R⁵)(R⁶))
R^{2A} is hydrogen, halo, hydroxyl, C₁₋₄ alkyl, C₁₋₄ alkoxyl, or —N(R^{5A})(R^{6A}); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

In certain embodiments, the TPL is one of the following:

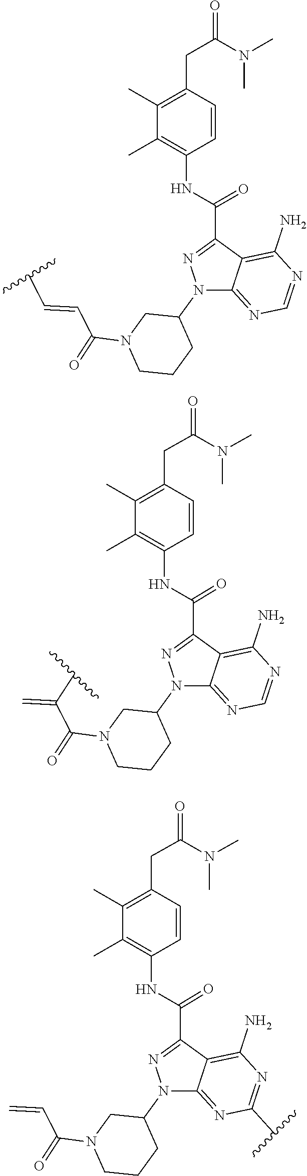

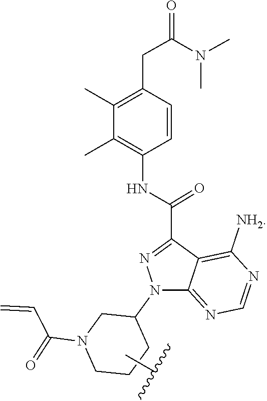

In certain embodiments, the TPL is

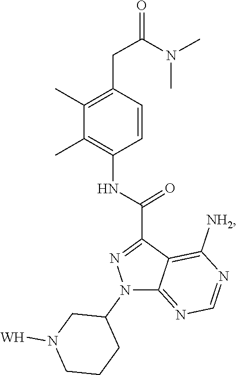

where WH is a group that reacts with HER2 to form a covalent linkage.

In certain embodiments, the TPL is one of the following:

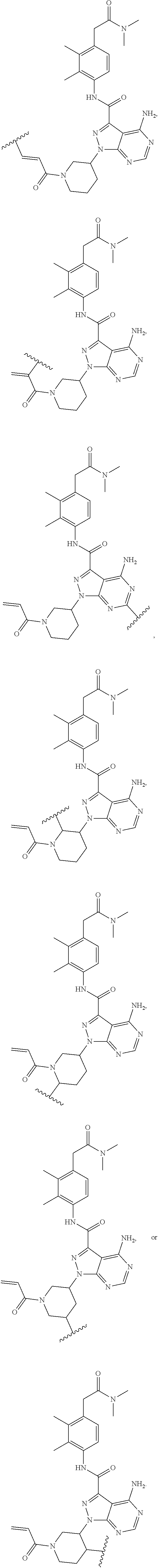

In certain embodiments, the TPL is a moiety that inhibits and/or binds human epidermal growth factor receptor 2 (HER2). Compounds that inhibit and/or bind to HER2 are reported in the literature, which include:

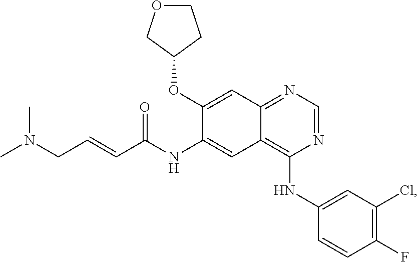 [SMILES: CN(C)C/C=C/C(=O)Nc1cc2c(Nc3ccc(F)c(Cl)c3)ncnc2cc1O[C@H]1CCOC1]

as described in Chen, J. et al., WO 2015/023703;

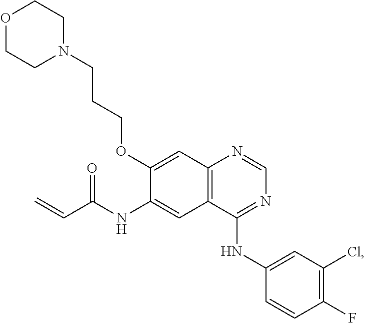 [SMILES: C=CC(=O)Nc1cc2c(Nc3ccc(F)c(Cl)c3)ncnc2cc1OCCCN1CCOCC1]

as described in Huang, Z. et al., WO 2012/027960;

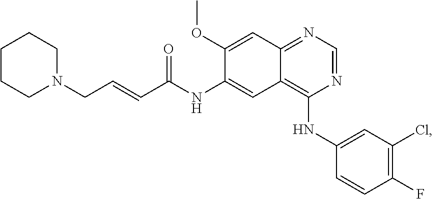 [SMILES: COc1cc2ncnc(Nc3ccc(F)c(Cl)c3)c2cc1NC(=O)/C=C/CN1CCCCC1]

as described in Wu, F. et al., WO 2012/159457;

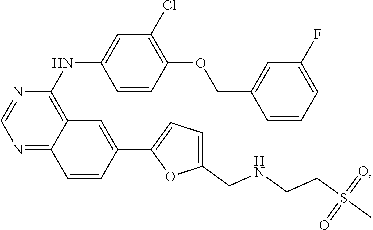 [SMILES: CS(=O)(=O)CCNCc1ccc(-c2ccc3ncnc(Nc4ccc(OCc5cccc(F)c5)c(Cl)c4)c3c2)o1]

as described in Wu, F. et al., WO 2012/159457;

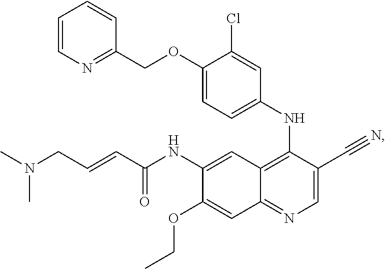 [SMILES: CCOc1cc2ncc(C#N)c(Nc3ccc(OCc4ccccn4)c(Cl)c3)c2cc1NC(=O)/C=C/CN(C)C]

as described in Wissner, A. et al., WO 2005/034955;

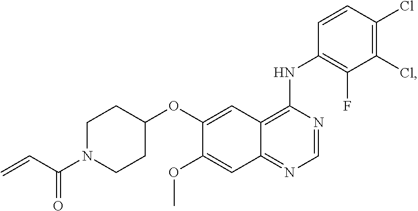 [SMILES: C=CC(=O)N1CCC(Oc2cc3c(Nc4ccc(Cl)c(Cl)c4F)ncnc3cc2OC)CC1]

as described in Li, Z. et al., WO 2019/149164;

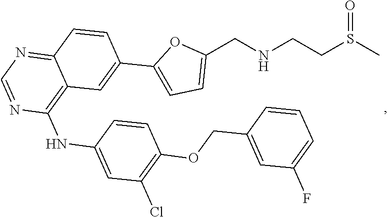 [SMILES: CS(=O)CCNCc1ccc(-c2ccc3ncnc(Nc4ccc(OCc5cccc(F)c5)c(Cl)c4)c3c2)o1]

as described in Wang, J. et al., WO 2011/035540;

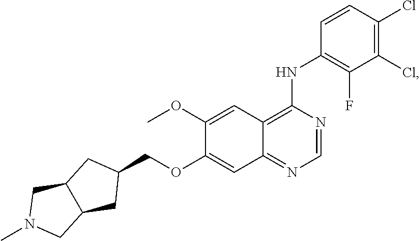 [SMILES: COc1cc2c(Nc3ccc(Cl)c(Cl)c3F)ncnc2cc1OC[C@@H]1C[C@@H]2CN(C)C[C@@H]2C1]

as described in Frost, P. et al., WO 2012/027537;

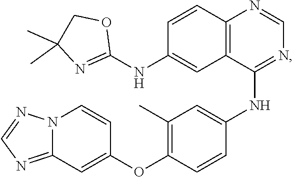 [SMILES: Cc1cc(Nc2ncnc3ccc(NC4=NC(C)(C)CO4)cc23)ccc1Oc1ccn2ncnc2c1]

as described in Xia, G. et al., WO 2017/148391;

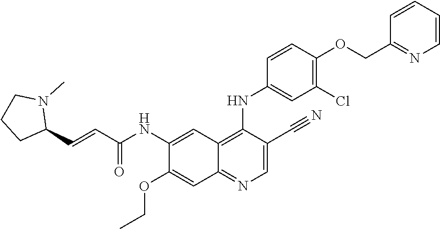 [SMILES: CCOc1cc2ncc(C#N)c(Nc3ccc(OCc4ccccn4)c(Cl)c3)c2cc1NC(=O)/C=C/[C@H]1CCCN1C]

as described in Li, X. et al., WO 2012/122865.

In certain embodiments, the TPL is a radical of one of the above compounds, which is attached to L through a modifiable oxygen, nitrogen, or carbon atom.

### Moiety for EGFR

In certain embodiments, the TPL is a moiety that binds to EGFR. Exemplary compounds that bind to EGFR are reported in the literature, such as Osimertinib and mavelertinib. A radical of such compounds reported in the literature that bind EGFR are amenable for use in the present invention.

In certain embodiments, the TPL is one of the following:

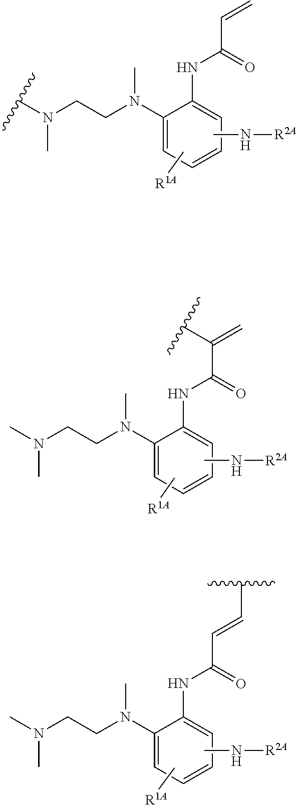

wherein:
R^{1A} is hydrogen, halo, hydroxyl, C₁₋₄ alkyl, C₁₋₄ alkoxyl, or N(R^{5A})(R^{6A});
R^{2A} is -(5-12 membered heteroaryl containing 1, 2, or 3 heteroatoms independently selected from nitrogen, oxygen and sulfur, wherein said heteroaryl is optionally substituted with 1, 2, 3, or 5 substituents independently selected from halo, hydroxyl, C₁₋₄ alkyl, and C₁₋₄ alkoxyl)-(5-12 membered heteroaryl containing 1, 2, or 3 heteroatoms independently selected from nitrogen, oxygen and sulfur, wherein said heteroaryl is optionally substituted with 1, 2, 3, or 5 substituents independently selected from halo, hydroxyl, C₁₋₄ alkyl, and C₁₋4 alkoxyl);
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

In certain embodiments, the TPL is one of the following:

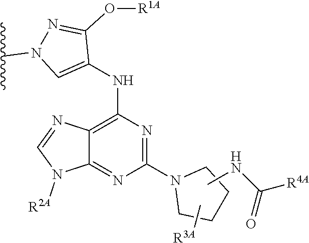

wherein: R^{1A} is C₁₋₄ alkyl; R^{2A} is hydrogen or C₁₋₄ alkyl; R^{3A} is halo; and R^{4A} is C₂₋₆ alkenyl.

In certain embodiments, TPL is

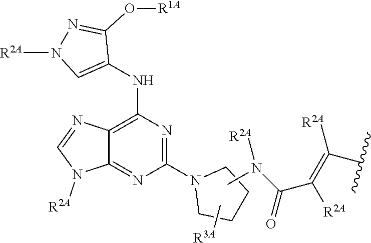

wherein R^{1A} is C₁₋₄ alkyl; R^{2A} represents independently for each occurrence hydrogen or C₁₋₄ alkyl; and R^{3A} is halo.

In certain embodiments, the TPL is one of the following:

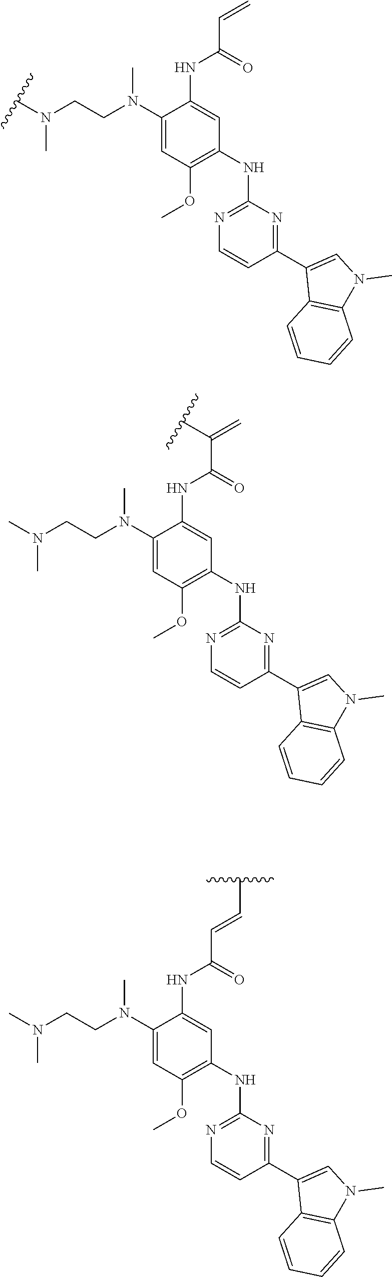

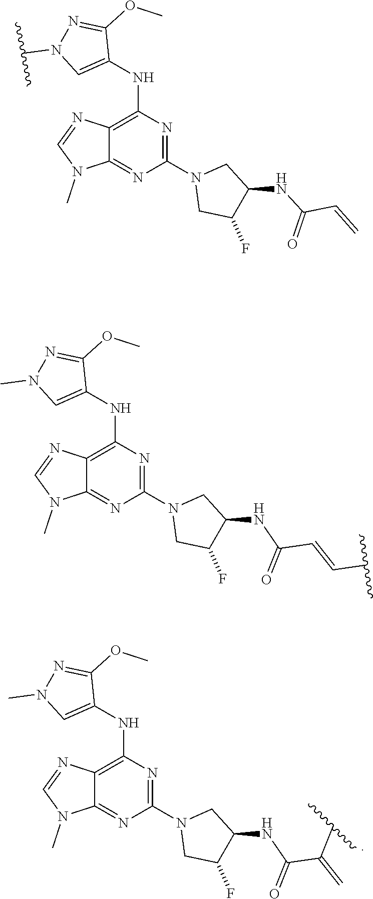

In certain embodiments, the TPL is one of the following:

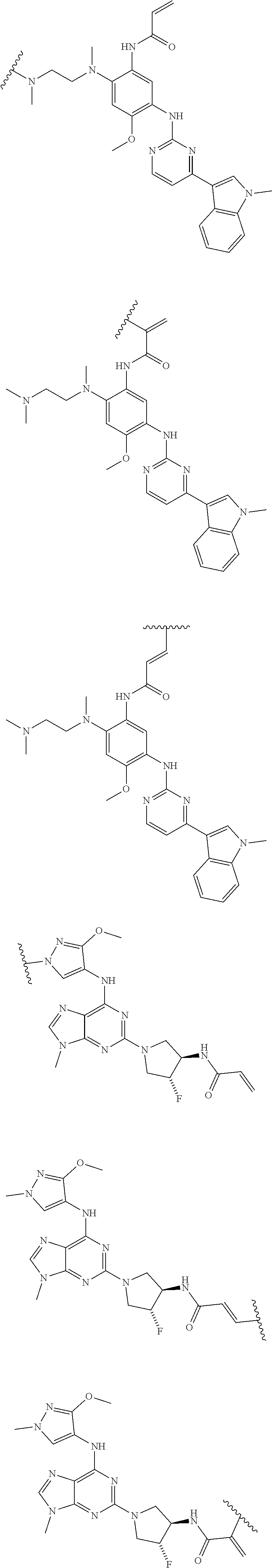

In certain embodiments, the TPL is

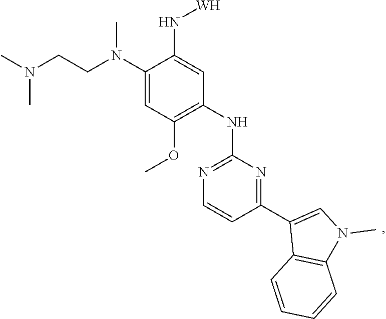

wherein WH is a group that reacts with EGFR to form a covalent linkage. In certain embodiments, the TPL is one of the following:

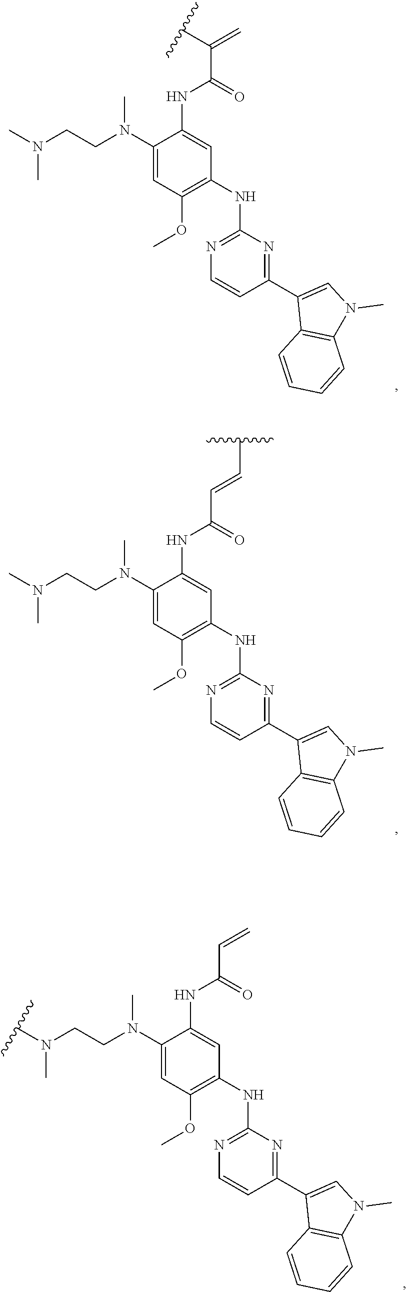

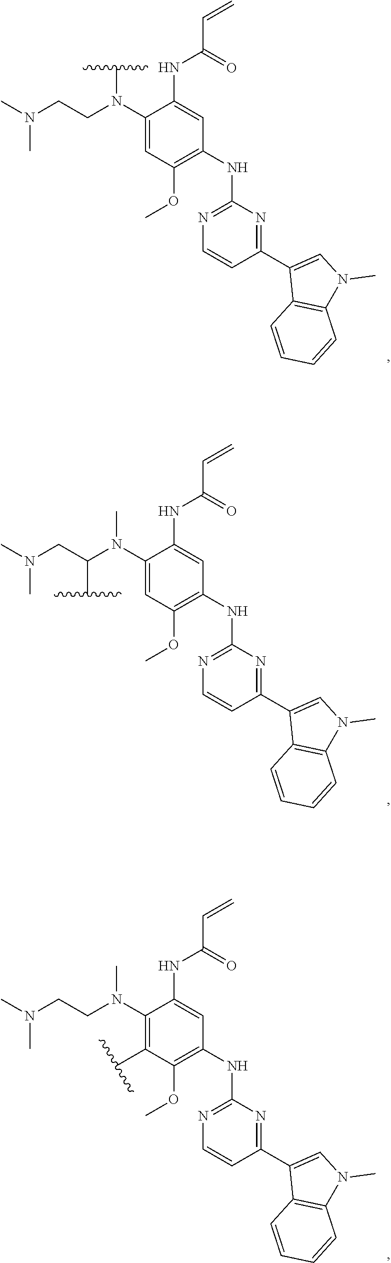

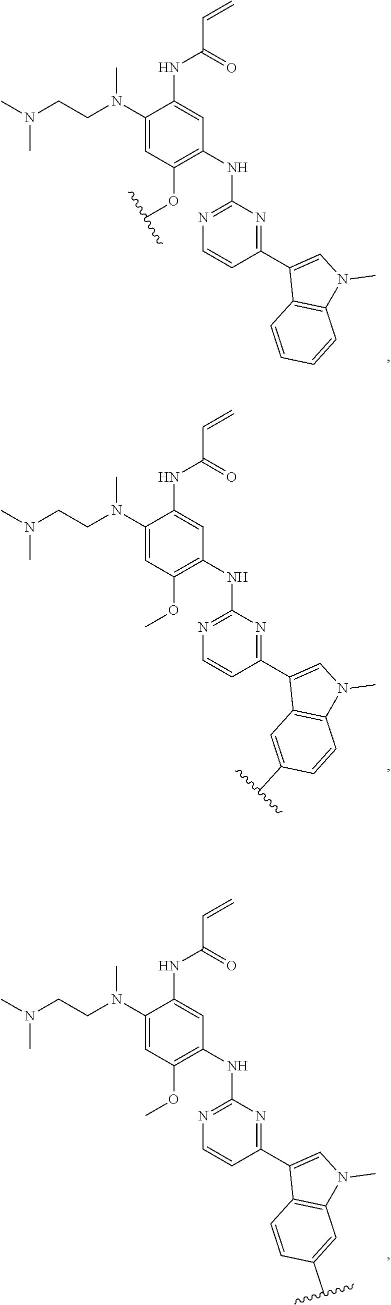

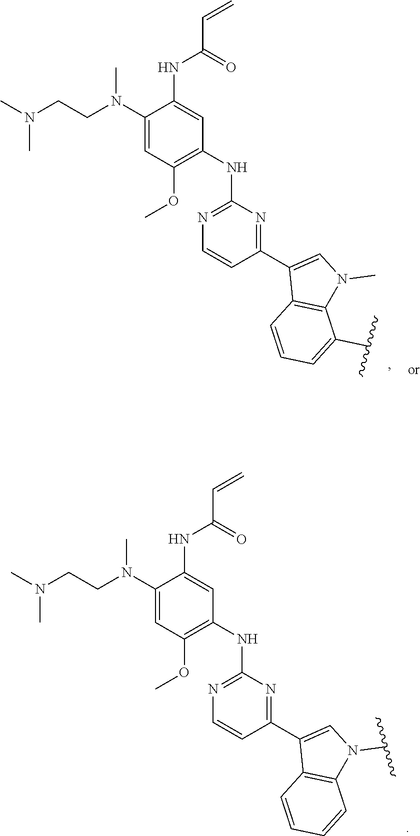

In certain embodiments, the TPL is

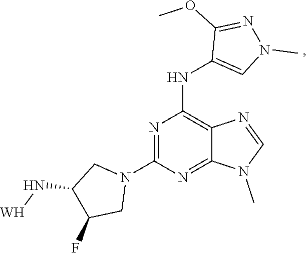

wherein WH is a group that reacts with EGFR to form a covalent linkage. In certain embodiments, the TPL is one of the following:

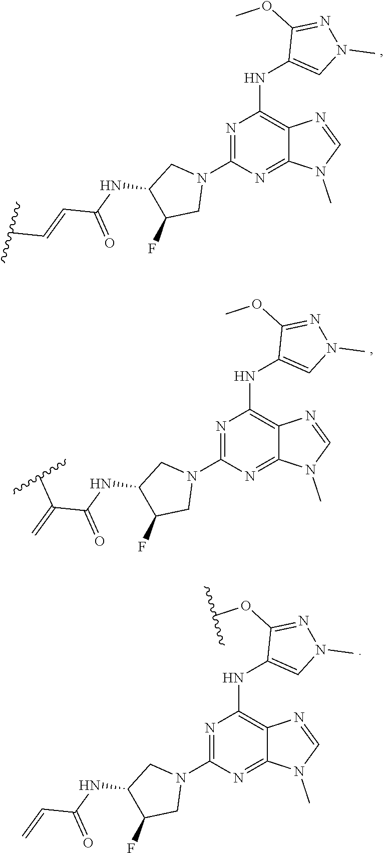

In certain embodiments, the TPL is

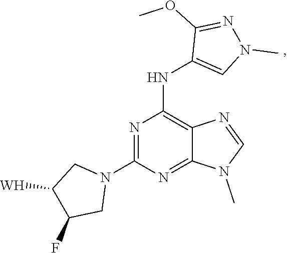

wherein WH is a group that reacts with EGFR to form a covalent linkage.

In certain embodiments, the TPL is a moiety that inhibits or binds to epidermal growth factor receptor (EGFR). Compounds that inhibitor or bind to EGFR are reported in the literature, which include:

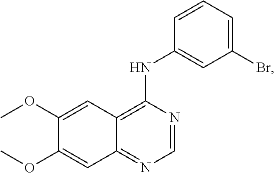 [SMILES: COc1cc2ncnc(Nc3cccc(Br)c3)c2cc1OC]

as described in Gangjee, A. et al., WO 2012/106522;

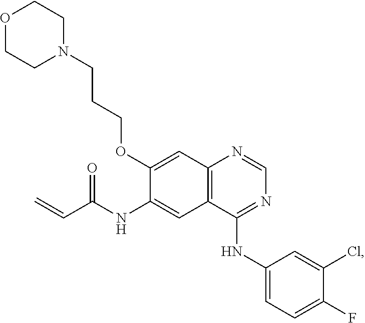 [SMILES: C=CC(=O)Nc1cc2c(Nc3ccc(F)c(Cl)c3)ncnc2cc1OCCCN1CCOCC1]

as described in Huang, Z. et al., WO 2012/027960;

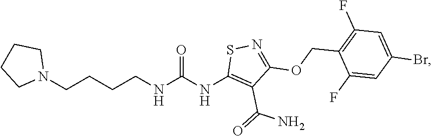 [SMILES: NC(=O)c1c(OCc2c(F)cc(Br)cc2F)nsc1NC(=O)NCCCCN1CCCC1]

as described in Bingaman, D. P. et al., WO 2014/152661;

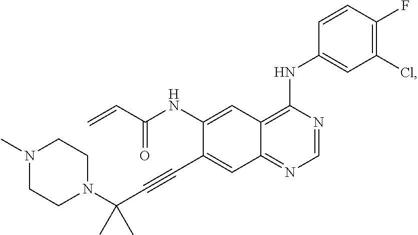 [SMILES: C=CC(=O)Nc1cc2c(Nc3ccc(F)c(Cl)c3)ncnc2cc1C#CC(C)(C)N1CCN(C)CC1]

as described in Kitano, Y. et al., WO 2002/066445;

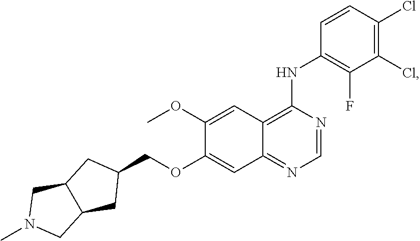 [SMILES: COc1cc2c(Nc3ccc(Cl)c(Cl)c3F)ncnc2cc1OC[C@@H]1C[C@@H]2CN(C)C[C@@H]2C1]

as described in Frost, P. et al., WO 2012/027537;

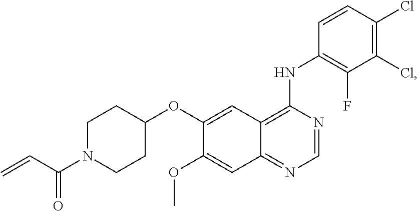 [SMILES: C=CC(=O)N1CCC(Oc2cc3c(Nc4ccc(Cl)c(Cl)c4F)ncnc3cc2OC)CC1]

as described in Lee, K.-O. et al., WO 2008/150118;

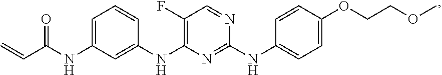 [SMILES: C=CC(=O)Nc1cccc(Nc2nc(Nc3ccc(OCCOC)cc3)ncc2F)c1 ; ","]

as described in Kluge, A. F. et al., WO 2009/158571;

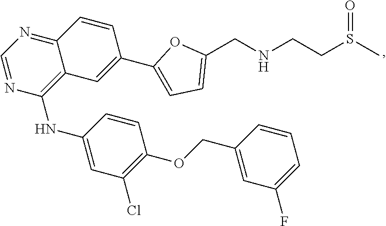 [SMILES: CS(=O)CCNCc1ccc(-c2ccc3ncnc(Nc4ccc(OCc5cccc(F)c5)c(Cl)c4)c3c2)o1 ; ","]

as described in Wang, J. et al., WO 2011/035540;

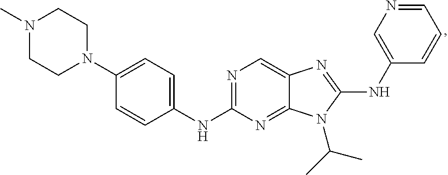 [SMILES: CC(C)n1c(Nc2cccnc2)nc2cnc(Nc3ccc(N4CCN(C)CC4)cc3)nc21 ; ","]

as described in Yang, S. et al., WO 2011/147066;

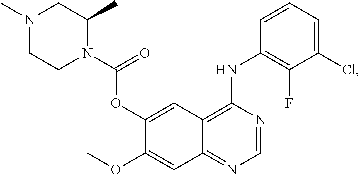 [SMILES: COc1cc2ncnc(Nc3cccc(Cl)c3F)c2cc1OC(=O)N1CCN(C)C[C@H]1C]

as described in Li, D. Y. et al., WO 2014/135876;

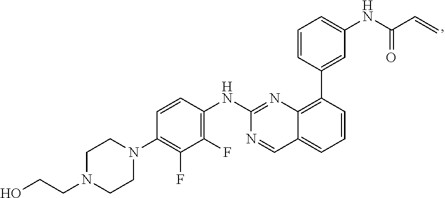 [SMILES: C=CC(=O)Nc1cccc(-c2cccc3cnc(Nc4ccc(N5CCN(CCO)CC5)c(F)c4F)nc23)c1 ; ","]

as described in Qian, X. et al., WO 2015/027222;

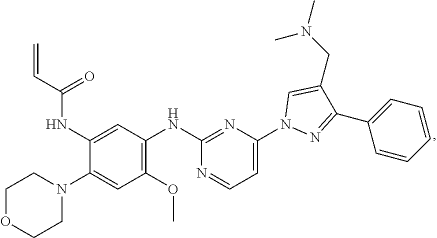 [SMILES: C=CC(=O)Nc1cc(Nc2nccc(-n3cc(CN(C)C)c(-c4ccccc4)n3)n2)c(OC)cc1N1CCOCC1 ; ","]

as described in Suh, B.-C. et al., WO 2016/060443;

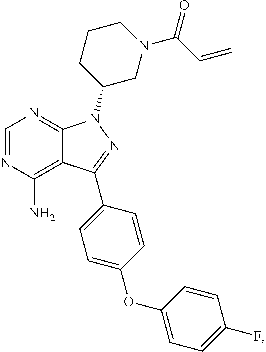 [SMILES: C=CC(=O)N1CCC[C@@H](n2nc(-c3ccc(Oc4ccc(F)cc4)cc3)c3c(N)ncnc32)C1]

as described in Zhang, D. et al., WO 2014/187319;

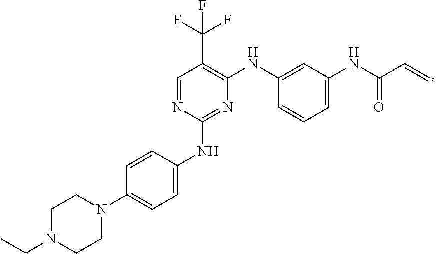 [SMILES: C=CC(=O)Nc1cccc(Nc2nc(Nc3ccc(N4CCN(CC)CC4)cc3)ncc2C(F)(F)F)c1 ; ","]

as described in Zhang, D. et al., WO 2015/117547;

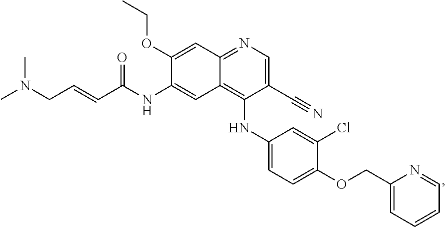 [SMILES: CCOc1cc2ncc(C#N)c(Nc3ccc(OCc4ccccn4)c(Cl)c3)c2cc1NC(=O)/C=C/CN(C)C ; ","]

as described in Wissner, A. et al., WO 2005/059678;

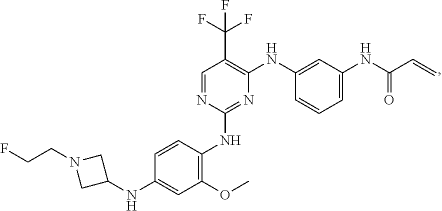 [SMILES: C=CC(=O)Nc1cccc(Nc2nc(Nc3ccc(NC4CN(CCF)C4)cc3OC)ncc2C(F)(F)F)c1 ; ","]

as described in Lee, K. et al., WO 2012/064706.

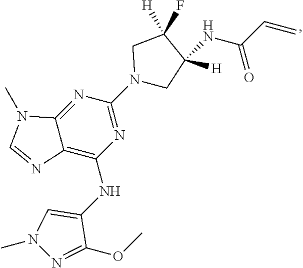 [SMILES: C=CC(=O)N[C@@H]1CN(c2nc(Nc3cn(C)nc3OC)c3ncn(C)c3n2)C[C@H]1F ; ","]

as described in Behenna, D. et al., WO 2015/075598.

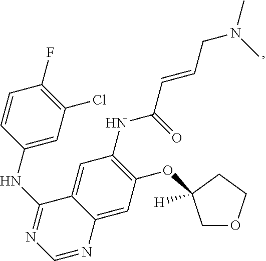 [SMILES: CN(C)C/C=C/C(=O)Nc1cc2c(Nc3ccc(F)c(Cl)c3)ncnc2cc1O[C@H]1CCOC1 ; ","]

as described in Himmelsbach, F. et al., WO 2002/050043.

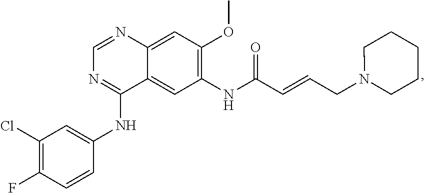 [SMILES: COc1cc2ncnc(Nc3ccc(F)c(Cl)c3)c2cc1NC(=O)/C=C/CN1CCCCC1 ; ","]

as described in Fakhoury, S. et al., WO 2005/107758.

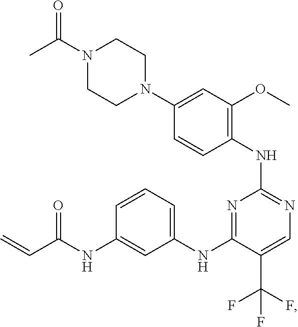 [SMILES: C=CC(=O)Nc1cccc(Nc2nc(Nc3ccc(N4CCN(C(C)=O)CC4)cc3OC)ncc2C(F)(F)F)c1]

as described in Lee, K. et al., WO 2012/061299.

In certain embodiments, the TPL is a radical of one of the above compounds, which is attached to L through a modifiable oxygen, nitrogen, or carbon atom.

### Moiety for KRas

In certain embodiments, the TPL is a moiety that binds to KRas. Exemplary compounds that bind to KRas are reported in the literature, such as MRTX849 and AMG510. A radical of such compounds reported in the literature that bind KRas are amenable for use in the present invention.

In certain embodiments, the TPL is one of the following:

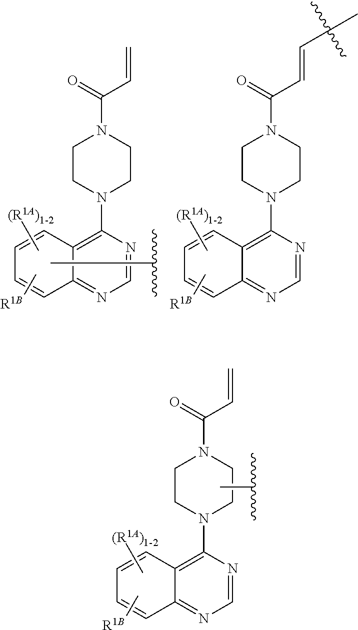

wherein:
R^{1A} represents independently for each occurrence hydrogen, halo, hydroxyl, C₁₋₄ alkyl, or C₁₋₄ alkoxyl; and
R^{1B} is C₆-12 aryl optionally substituted by 1, 2, or 3 substituents independently selected from halo, hydroxyl, C₁₋₄ alkyl, or C₁₋₄ alkoxyl.

In certain embodiments, the TPL is one of the following:

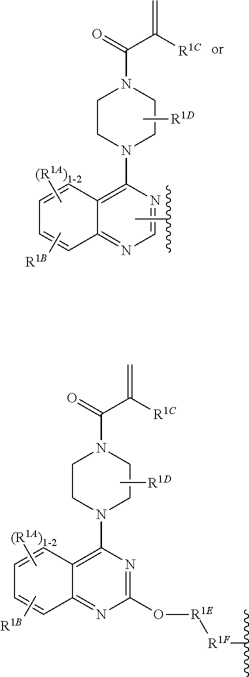

wherein:
R^{1A} represents independently for each occurrence hydrogen, halo, hydroxyl, C₁₋₄ alkyl, or C₁₋₄ alkoxyl;
R^{1B} is C₆₋₁₂ aryl optionally substituted by 1, 2, or 3 substituents independently selected from halo, hydroxyl, C₁₋₄ alkyl, or C₁₋₄ alkoxyl;
R^{1C} represents independently for each occurrence hydrogen, halo, or C₁₋₄ alkyl;
R^{1D} represents independently for each occurrence (C₁₋₆ alkylene)-CN;
R^{1E} is C₁₋₆ alkylene; and
R^{1F} is 3-6 membered saturated, monocyclic heterocyclylene containing 1, 2, or 3 heteroatoms independently selected from oxygen, nitrogen, and sulfur.

In certain embodiments, the TPL is one of the following:

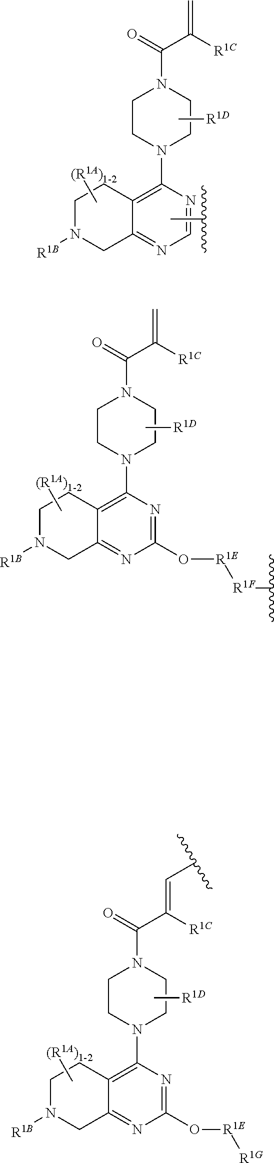

wherein:
R^{1A} represents independently for each occurrence hydrogen, halo, hydroxyl, C₁₋₄ alkyl, or C₁₋₄ alkoxyl;
R^{1B} is C₆-12 aryl optionally substituted by 1, 2, or 3 substituents independently selected from halo, hydroxyl, C₁₋₄ alkyl, or C₁₋₄ alkoxyl.
R^{1C} represents independently for each occurrence hydrogen, halo, or C₁₋₄ alkyl;
R^{1D} represents independently for each occurrence (C₁₋₆ alkylene)-CN;
R^{1E} is C₁₋₆ alkylene; and
R^{1F} is 3-6 membered saturated, monocyclic heterocyclylene containing 1, 2, or 3 heteroatoms independently selected from oxygen, nitrogen, and sulfur, wherein the heterocyclylene is substituted with 1 or 2 occurrences of R^{1A};
R^{1G} is 3-6 membered saturated, monocyclic heterocyclyl containing 1, 2, or 3 heteroatoms independently selected from oxygen, nitrogen, and sulfur, wherein the heterocyclyl is substituted with 1 or 2 occurrences of R^{1A}.

In certain embodiments, the TPL is

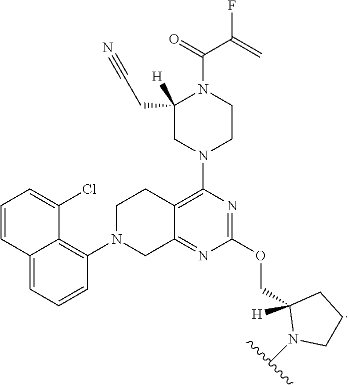 [SMILES: *N1CCC[C@H]1COc1nc2c(c(N3CCN(C(=O)C(=C)F)[C@@H](CC#N)C3)n1)CCN(c1cccc3cccc(Cl)c13)C2 ; "."]

In certain embodiments, the the TPL is one of the following:

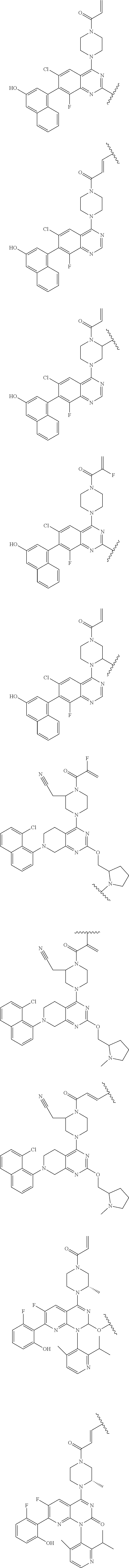

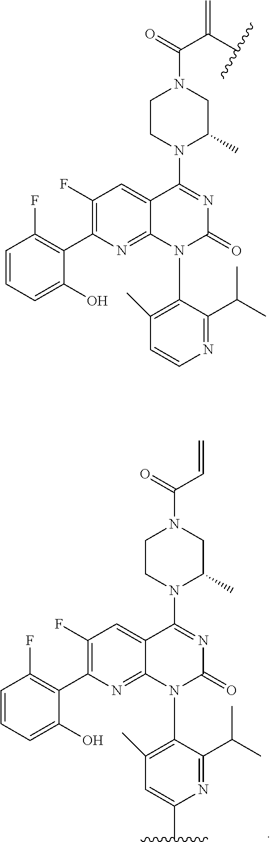 [SMILES: *C(=C)C(=O)N1CCN(c2nc(=O)n(-c3c(C)ccnc3C(C)C)c3nc(-c4c(O)cccc4F)c(F)cc23)[C@@H](C)C1 ; *c1cc(C)c(-n2c(=O)nc(N3CCN(C(=O)C=C)C[C@@H]3C)c3cc(F)c(-c4c(O)cccc4F)nc32)c(C(C)C)n1]

In certain embodiments, the the TPL is one of the following:

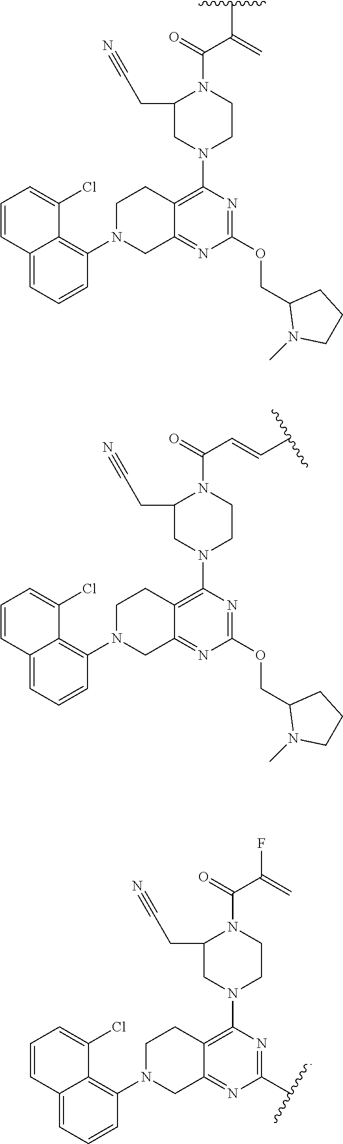 [SMILES: *C(=C)C(=O)N1CCN(c2nc(OCC3CCCN3C)nc3c2CCN(c2cccc4cccc(Cl)c24)C3)CC1CC#N ; */C=C/C(=O)N1CCN(c2nc(OCC3CCCN3C)nc3c2CCN(c2cccc4cccc(Cl)c24)C3)CC1CC#N ; *c1nc2c(c(N3CCN(C(=O)C(=C)F)C(CC#N)C3)n1)CCN(c1cccc3cccc(Cl)c13)C2 ; "."]

In certain embodiments, the TPL is

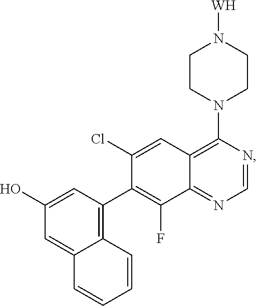

wherein WH is a group that reacts with KRas to form a covalent linkage. In certain embodiments, the TPL is one of the following:

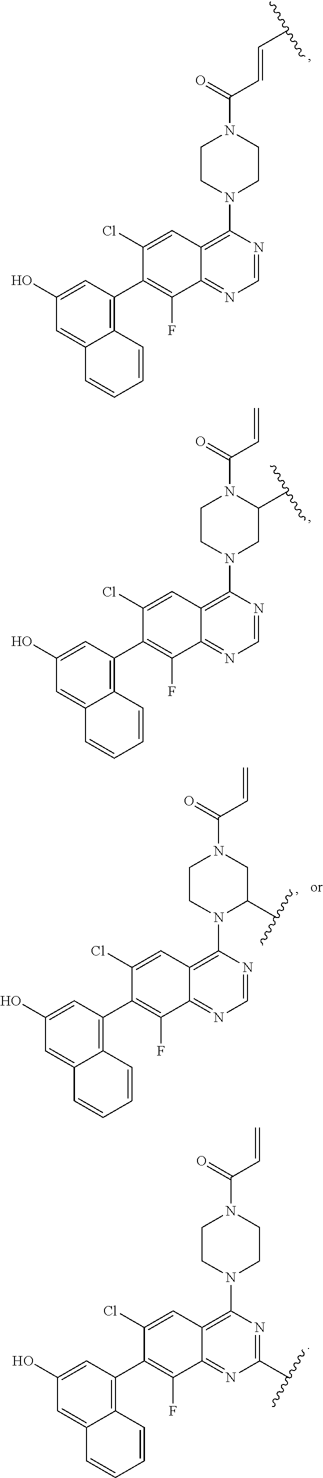

In certain embodiments, the TPL is

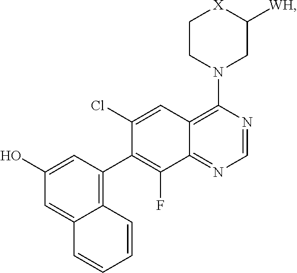

wherein X═NH, NR^{a}, CH₂, CHR^{a}, or C(R^{a})₂, R^{a} is C₁₋₆ alkyl, C₂₋₆ alkenyl, amido, amino, aminoalky, or C₁₋₆ alkoxy, and WH is a group that reacts with KRas to form a covalent linkage.

In certain embodiments, the TPL is

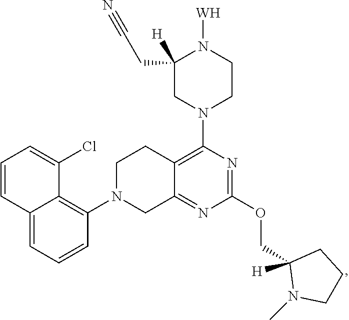

wherein WH is a group that reacts with KRas to form a covalent linkage.

In certain embodiments, the TPL is

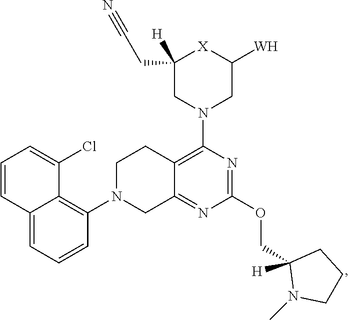

wherein WH is a group that reacts with KRas to form a covalent linkage; X is NH, NR^{a}, CH₂, CHR^{a}, or C(R^{a})₂; and R^{a} is C₁₋₆ alkyl, C₂₋₆ alkenyl, amido, amino, aminoalky, or C₁₋₆ alkoxy.

In certain embodiments, the TPL is

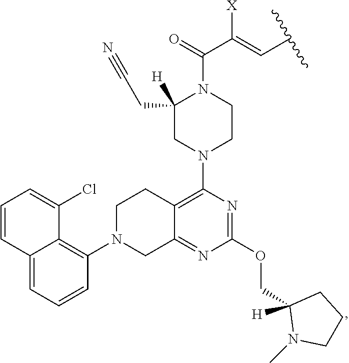

wherein WH is a group that reacts with KRas to form a covalent linkage; X is hydrogen or halo.

In certain embodiments, the TPL is one of the following:

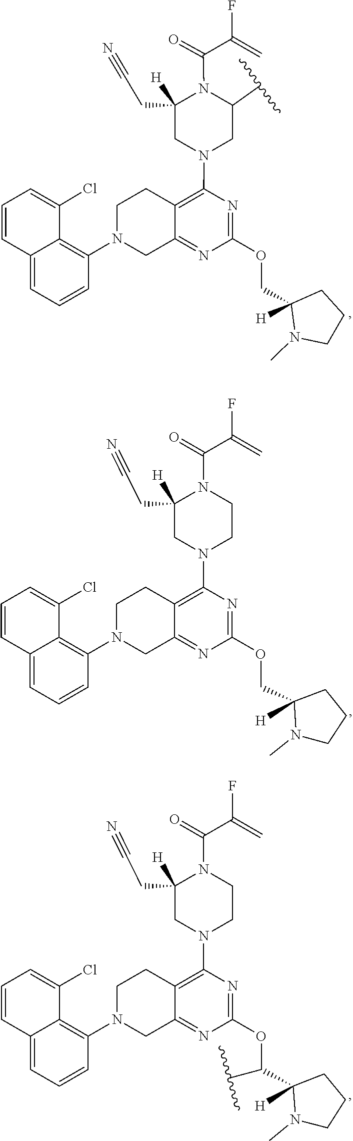 [SMILES: *C1CN(c2nc(OC[C@@H]3CCCN3C)nc3c2CCN(c2cccc4cccc(Cl)c24)C3)C[C@H](CC#N)N1C(=O)C(=C)F ; C=C(F)C(=O)N1CCN(c2nc(OC[C@@H]3CCCN3C)nc3c2CCN(c2cccc4cccc(Cl)c24)C3)C[C@@H]1CC#N ; *C(Oc1nc2c(c(N3CCN(C(=O)C(=C)F)[C@@H](CC#N)C3)n1)CCN(c1cccc3cccc(Cl)c13)C2)[C@@H]1CCCN1C]

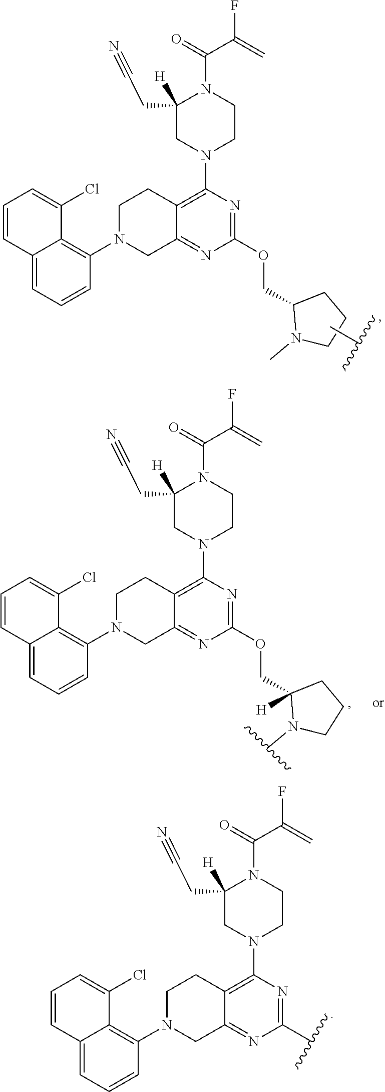

In certain embodiments, the TPL is

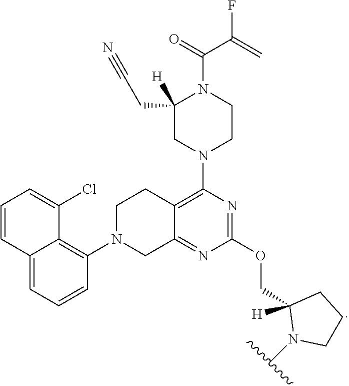 [SMILES: *N1CCC[C@H]1COc1nc2c(c(N3CCN(C(=O)C(=C)F)[C@@H](CC#N)C3)n1)CCN(c1cccc3cccc(Cl)c13)C2 ; "."]

In certain embodiments, the TPL is a radical of

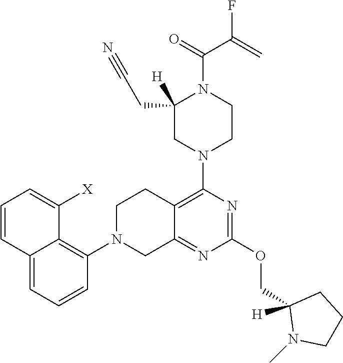

wherein X is hydrogen, halo, C₁₋₆ alkyl, amino or C₁₋₆ alkoxy; and the TPL is attached to through a modifiable oxygen, nitrogen, or carbon atom.

In certain embodiments, the TPL is

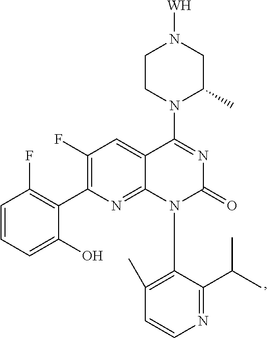

wherein WH is a group that reacts with KRas to form a covalent linkage.

In certain embodiments, the TPL is

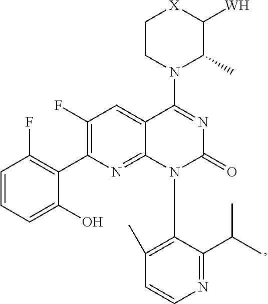

wherein WH is a group that reacts with KRas to form a covalent linkage; X is NH, NR^{a}, CH₂, CHR^{a}, or C(R^{a})₂; and R^{a} is C₁₋₆ alkyl, C₂₋₆ alkenyl, amido, amino, aminoalkyl, or C₁₋₆ alkoxy.

In certain embodiments, the TPL is

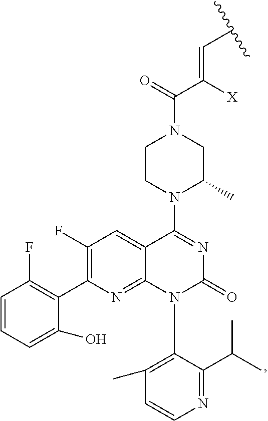

wherein X is hydrogen or halo. In certain embodiments, the TPL is

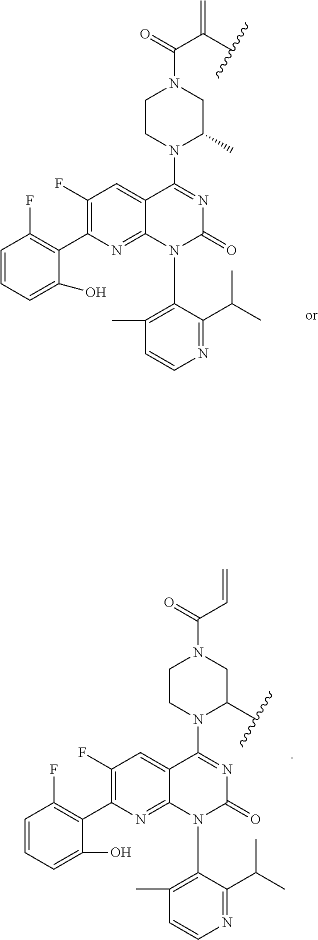 [SMILES: *C(=C)C(=O)N1CCN(c2nc(=O)n(-c3c(C)ccnc3C(C)C)c3nc(-c4c(O)cccc4F)c(F)cc23)[C@@H](C)C1 ; *C1CN(C(=O)C=C)CCN1c1nc(=O)n(-c2c(C)ccnc2C(C)C)c2nc(-c3c(O)cccc3F)c(F)cc12]

In certain embodiments, the TPL is one of the following:

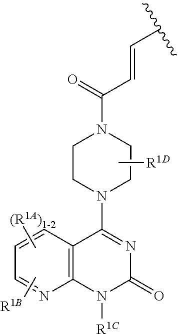

wherein:
R^{1A} represents independently for each occurrence hydrogen, halo, hydroxyl, C₁₋₄ alkyl, or C₁₋₄ alkoxyl;
R^{1B} and R^{1C} are independently phenyl optionally substituted by 1, 2, or 3 substituents independently selected from halo, hydroxyl, C₁₋₄ alkyl, or C₁₋₄ alkoxyl;
R^{1D} is hydrogen or C₁₋₄ alkyl;

In certain embodiments, the TPL is

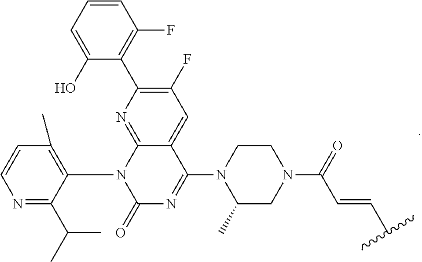 [SMILES: */C=C/C(=O)N1CCN(c2nc(=O)n(-c3c(C)ccnc3C(C)C)c3nc(-c4c(O)cccc4F)c(F)cc23)[C@@H](C)C1]

In certain embodiments, the TPL is

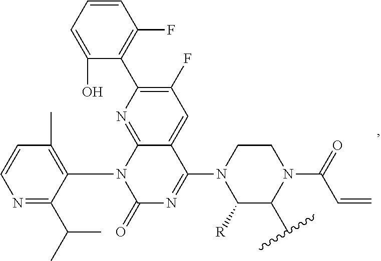

wherein R═H, Me, Et, CH₂OH, CH₂NH₂, CH₂NHR′, OH, or NH₂; and R′ is alkyl, alkenyl, amido, amino, aminoalky, or alkoxy. In certain embodiments, the TPL is

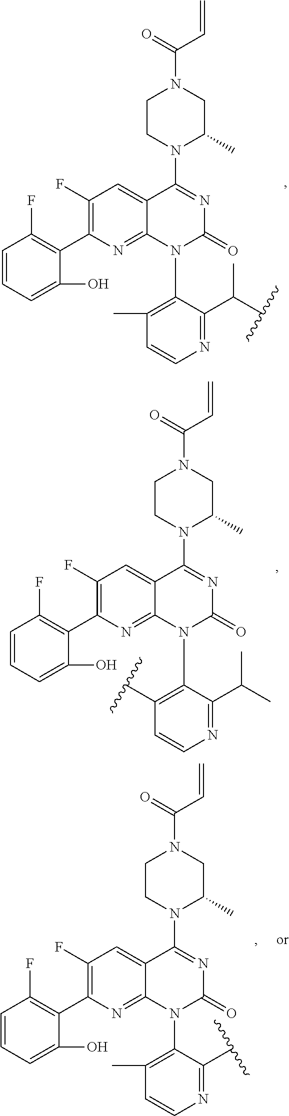

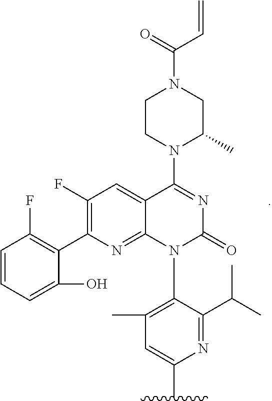 [SMILES: *c1cc(C)c(-n2c(=O)nc(N3CCN(C(=O)C=C)C[C@@H]3C)c3cc(F)c(-c4c(O)cccc4F)nc32)c(C(C)C)n1]

In certain embodiments, the TPL is

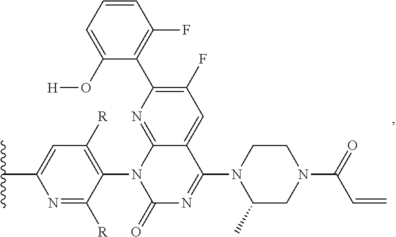

wherein R is H, a linker (e.g., alkyl); and R′ is H or a linker (e.g., alkyl). In certain embodiments, the TPL is

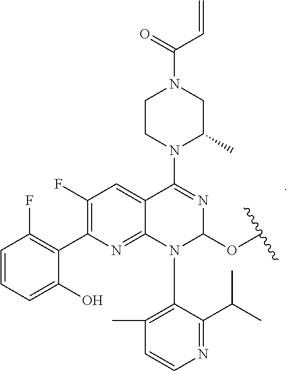 [SMILES: *OC1N=C(N2CCN(C(=O)C=C)C[C@@H]2C)c2cc(F)c(-c3c(O)cccc3F)nc2N1c1c(C)ccnc1C(C)C]

In certain embodiments, the TPL is

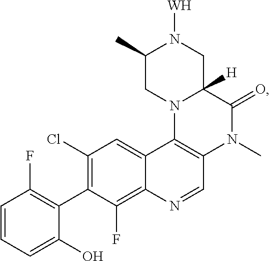

wherein WH is a group that reacts with KRas to form a covalent linkage. In certain embodiments, the TPL is

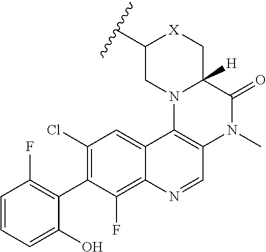

wherein X is NH, NR^{a}, CH₂, CHR^{a}, or C(R^{a})₂; and R^{a} is alkyl, alkenyl, amido, amino, aminoalky, or alkoxy.

In certain embodiments, the TPL is

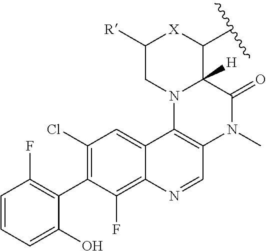

wherein X is NH, NR^{a}, CH₂, CHR^{a}, or C(R^{a})₂; R^{a} is alkyl, alkenyl, amido, amino, aminoalky, or alkoxy; and R′ is H, Me, or Et. In certain embodiments, the TPL is

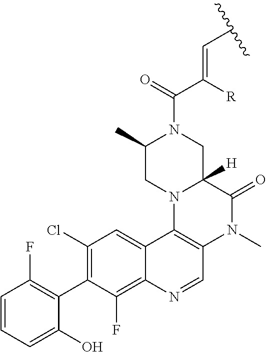

wherein R is hydrogen or halo. In certain embodiments, the TPL is

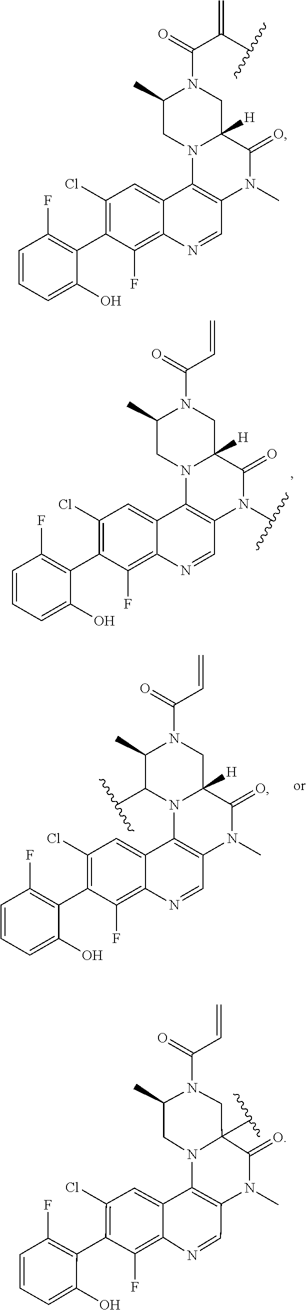 [SMILES: *C(=C)C(=O)N1C[C@@H]2C(=O)N(C)c3cnc4c(F)c(-c5c(O)cccc5F)c(Cl)cc4c3N2C[C@H]1C ; *N1C(=O)[C@H]2CN(C(=O)C=C)[C@H](C)CN2c2c1cnc1c(F)c(-c3c(O)cccc3F)c(Cl)cc21 ; *C1[C@@H](C)N(C(=O)C=C)C[C@@H]2C(=O)N(C)c3cnc4c(F)c(-c5c(O)cccc5F)c(Cl)cc4c3N12 ; *C12CN(C(=O)C=C)[C@H](C)CN1c1c(cnc3c(F)c(-c4c(O)cccc4F)c(Cl)cc13)N(C)C2=O]

In certain embodiments, the TPL is a moiety that binds to a mutated Kirsten rat sarcoma 2 viral oncogene homolog. Compounds that bind mutated Kirsten rat sarcoma 2 viral oncogene homolog are reported in the literature, which include:

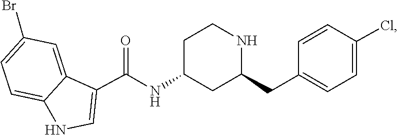 [SMILES: O=C(N[C@@H]1CCN[C@@H](Cc2ccc(Cl)cc2)C1)c1c[nH]c2ccc(Br)cc12]

as described in Jansen, J. M. et al., 24th Int Symp Med Chem (August 28-September 1, Manchester) 2016, Abstract LE007;

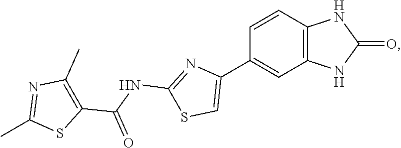 [SMILES: Cc1nc(C)c(C(=O)Nc2nc(-c3ccc4[nH]c(=O)[nH]c4c3)cs2)s1]

as described in Rabizadeh, S. et al., WO 2016/161361;

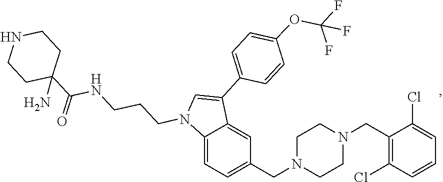 [SMILES: NC1(C(=O)NCCCn2cc(-c3ccc(OC(F)(F)F)cc3)c3cc(CN4CCN(Cc5c(Cl)cccc5Cl)CC4)ccc32)CCNCC1]

as described in Welsch, M. E. et al., Cell 2017, vol. 168(5), page 878;

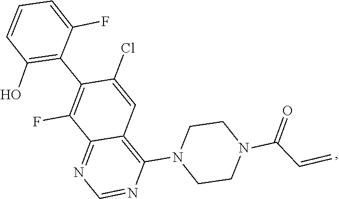 [SMILES: C=CC(=O)N1CCN(c2ncnc3c(F)c(-c4c(O)cccc4F)c(Cl)cc23)CC1]

as described in Wijeratne, A. et al., ACS Med Chem Lett 2018, vol. 9(6), page 557.

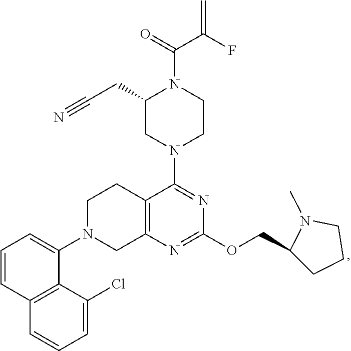 [SMILES: C=C(F)C(=O)N1CCN(c2nc(OC[C@@H]3CCCN3C)nc3c2CCN(c2cccc4cccc(Cl)c24)C3)C[C@@H]1CC#N]

as described in Blake, J. et al., WO 2019/099524

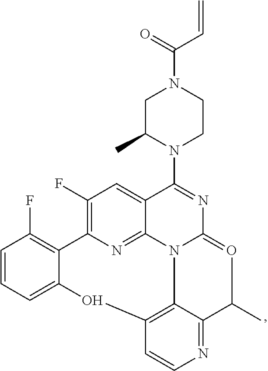

as described in Lanman, B. et al., WO 2018/217651

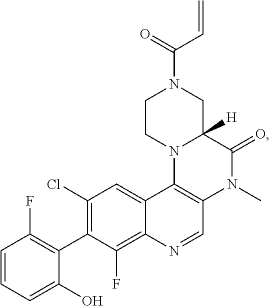 [SMILES: C=CC(=O)N1CCN2c3c(cnc4c(F)c(-c5c(O)cccc5F)c(Cl)cc34)N(C)C(=O)[C@H]2C1]

as described in Kettle, J. et al., WO 2019/110751.

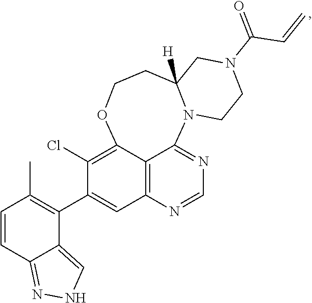 [SMILES: C=CC(=O)N1CCN2c3ncnc4cc(-c5c(C)ccc6n[nH]cc56)c(Cl)c(c34)OCC[C@@H]2C1]

as described in Kettle, J. et al., WO 2018/206539.

In certain embodiments, the TPL is a radical of one of the above compounds, which is attached to L through a modifiable oxygen, nitrogen, or carbon atom.

### Additional Features

Certain embodiments above describe compounds and/or moieties that contain a warhead (WH) group. In certain embodiments, the WH group is R^{WH}, which is an electrophilic group capable of reacting with a protein, such as reacting with a nucleophilic functional group of a protein, such as a sulfhydryl group of a cysteine residue or an amino group of a lysine residue.

In certain embodiments, R^{WH} is —L^{W}-Y^{W}, wherein:
L^{W} is a covalent bond or a bivalent C₁₋₈ saturated or unsaturated, straight or branched, hydrocarbon chain, wherein one, two, or three methylene units of L^{W} are optionally and independently replaced by cyclopropylene, —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, —C(═S)—, —C(═NH)—, —N═N—, or —C(═N₂); and
Y^{W} is —C(O)—(C₂₋₆ alkenyl), —C(O)—(C₂₋₆ fluoroalkenyl), —C(O)—(C₂₋₆ alkynyl), —S(O)₂—(C₂₋₆ alkenyl), —S(O)₂—(C₂₋₆ fluoroalkenyl), —S(O)₂—(C₂₋₆ alkynyl), —S(O)₂—F, C₁₋₆ chloroalkyl, C₁₋₆ bromoalkyl, —(C₂₋₆ nitroalkenyl), or chloroacetyl, each of which is optionally substituted.

In certain embodiments, R^{WH} is —L^{W}-Y^{W}, wherein:
L^{W} is a covalent bond or a bivalent C₁₋₈ saturated or unsaturated, straight or branched, hydrocarbon chain, wherein one, two, or three methylene units of L^{W} are optionally and independently replaced by cyclopropylene, —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, —C(═S)—, —C(═NH)—, —N═N—, or —C(═N₂); and Y^{W} is

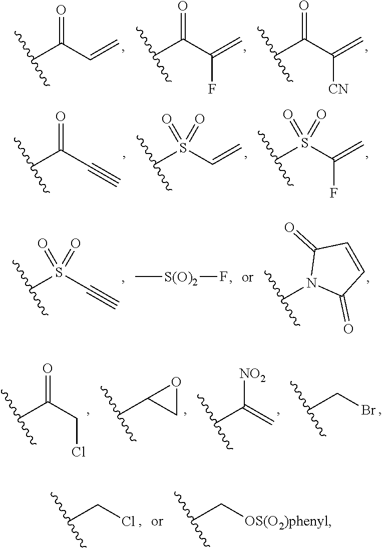

each of which is optionally substituted.

In certain embodiments, R^{WH} is —C(O)—(C₂₋₆ alkenyl), —C(O)—(C₂₋₆ fluoroalkenyl), —C(O)—(C₂₋₆ alkynyl), —S(O)₂—(C₂₋₆ alkenyl), —S(O)₂—(C₂₆ fluoroalkenyl), —S(O)₂—(C₂₋₆ alkynyl), —S(O)₂—F, C₁₋₆ chloroalkyl, C₁₋₆ bromoalkyl, —(C₂₋₆ nitroalkenyl), or chloroacetyl, each of which is optionally substituted. In certain embodiments, R^{WH} is

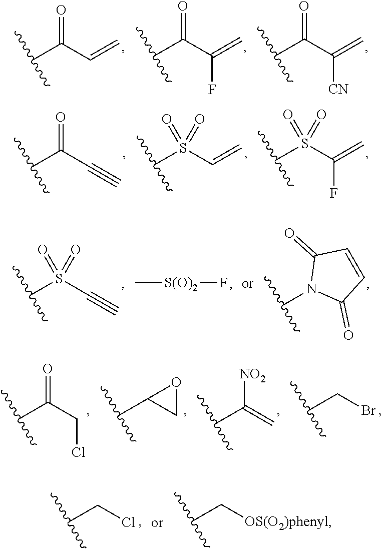

each of which is optionally substituted. In certain embodiments, R^{W} is

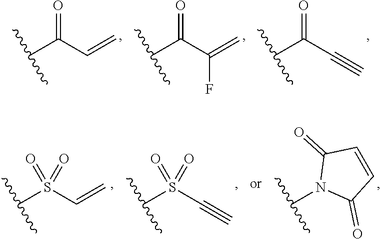

each of which is optionally substituted. In certain embodiments, R^{WH} is

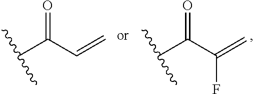

each of which is optionally substituted. In certain embodiments, R^{WH} is

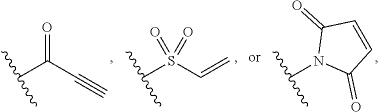

each of which is optionally substituted.

In certain embodiments, R^{WH} is —C(O)—(C₂₋₆ alkenyl), —C(O)—(C₂₋₆ fluoroalkenyl), —C(O)—(C₂₋₆ alkynyl), —S(O)₂—(C₂₋₆ alkenyl), —S(O)₂—(C₂₆ fluoroalkenyl), —S(O)₂—(C₂₋₆ alkynyl), —S(O)₂—F, C₁₋₆ chloroalkyl, C₁₋₆ bromoalkyl, —(C₂₋₆ nitroalkenyl), or chloroacetyl. In certain embodiments, R^{WH} is

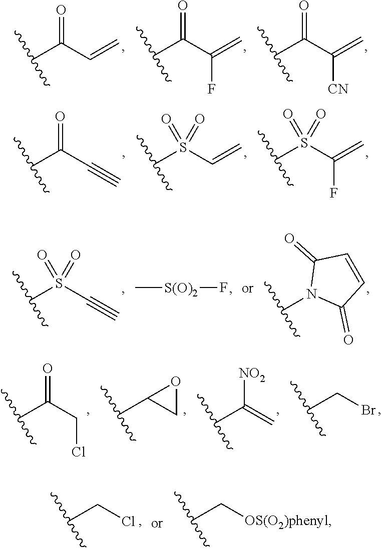

In certain embodiments, R^{WH} is

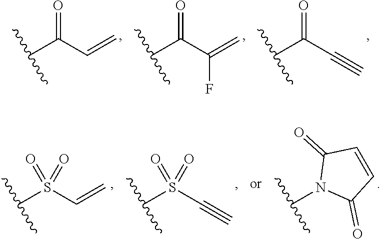

In certain embodiments, R^{WH} is

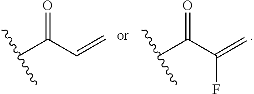

In certain embodiments, R^{W} is

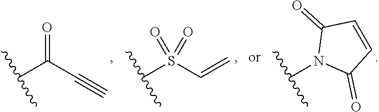

Compounds of Formula I may be further characterized according to the molecular weight of the TPL. In certain embodiments, the TPL has a molecular weight of less than 1500 Da, 1200 Da, 1000 Da, 800 Da, 600 Da, 400 Da, 300 Da, 200 Da, 150 Da, or 100 Da. Compounds of Formula II may be further characterized according to the molecular weight of the EPL. In certain embodiments, the EPL has a molecular weight of less than 1500 Da, 1200 Da, 1000 Da, 800 Da, 600 Da, 400 Da, 300 Da, 200 Da, 150 Da, or 100 Da.

### Part C: Exemplary Further Description of Linker (L) Component of Compounds of Formula I

Compounds of Formula I may be further characterized according to, for example, the identity of the linker (L) component. A variety of linkers are known to one of skill in the art and may be used in the heterobifunctional compounds described herein. For example, in certain embodiments, L comprises one or more optionally substituted groups selected from amino acids, polyether chains, aliphatic groups, and any combinations thereof. In certain embodiments, L consists of one or more optionally substituted groups selected from amino acids, polyether chains, aliphatic groups, and any combinations thereof. In certain embodiments, L consists of one or more groups selected from amino acids, polyether chains, aliphatic groups, and any combinations thereof.

In some embodiments, L is symmetrical. In some embodiments, L is asymmetric. In certain embodiments, L is a bond.

In certain embodiments, L is a covalent bond or a bivalent C₁-3₀ saturated or unsaturated, straight or branched, hydrocarbon chain, wherein 1-15 methylene units of L are optionally and independently replaced by cyclopropylene, —N(H)—, —N(C₁-4 alkyl)-, —N(C₃₋₅ cycloalkyl)-, —O—, —C(O)—, —OC(O)—, —C(O)O—, —S—, —S(O)—, —S(O)₂—, —S(O)₂N(H)—, —S(O)₂N(C₁₋₄ alkyl)-, —S(O)₂N(C₃₋₅ cycloalkyl)-, —N(H)C(O)—, —N(C₁₋₄ alkyl)C(O)—, —N(C₃₋₅ cycloalkyl)C(O)—, —C(O)N(H)—, —C(O)N(C₁₋₄ alkyl)-, —C(O)N(C₃₋₅ cycloalkyl)-, phenylene, an 8-10 membered bicyclic arylene, a 4-7 membered saturated or partially unsaturated carbocyclylene, an 8-10 membered bicyclic saturated or partially unsaturated carbocyclylene, a 3-7 membered saturated or partially unsaturated heterocyclylene having 1-2 heteroatoms independently selected from nitrogen, oxygen, and sulfur, an 8-10 membered bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur, wherein R** represents independently for each occurrence hydrogen, C₁₋₆ alkyl, or C₃₋₆ cycloalkyl.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

In certain embodiments, L is a bivalent, saturated or unsaturated, straight or branched C₁₋₆₀ hydrocarbon chain, wherein (i) 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur, and (ii) 0-1 methylene units of the hydrocarbon are independently replaced with —C(O)—(C₂₋₆ alkenylene)-, —C(O)—(C₂₋₆ fluoroalkenylene)-, —C(O)—(C₂₋₆ alkynylene)-, —S(O)₂—(C₂₋₆ alkenylene)-, —S(O)₂—(C₂₋₆ fluoroalkenylene)-, —S(O)₂—(C₂₋₆ alkynylene)-, or —(C₁₋₆ alkylene substituted with one R^{WH})—, wherein R^{WH}

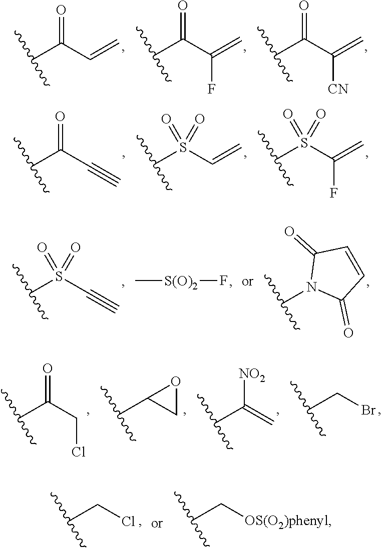

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, —C(O)N(C₁₋₆ alkyl)-, 3-10 membered carbocyclyl, or 3-10 membered heterocyclyl containing 1, 2, or 3 heteroatoms independently selected from nitrogen, oxygen, and sulfur.

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, or —C(O)N(C₁₋₆ alkyl)-.

In yet other embodiments, L comprises a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units. In yet other embodiments, L is a diradical of a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units.

In certain embodiments, L is a heteroalkylene having from 4 to 30 atoms selected from carbon, oxygen, nitrogen, and sulfur. In certain embodiments, L is a heteroalkylene having from 4 to 20 atoms selected from carbon, oxygen, nitrogen, and sulfur. In certain embodiments, L is a heteroalkylene having from 4 to 10 atoms selected from carbon, oxygen, nitrogen, and sulfur. In certain embodiments, L is a heteroalkylene having from 4 to 30 atoms selected from carbon, oxygen, and nitrogen. In certain embodiments, L is a heteroalkylene having from 4 to 20 atoms selected from carbon, oxygen, and nitrogen. In certain embodiments, L is a heteroalkylene having from 4 to 10 atoms selected from carbon, oxygen, and nitrogen. In certain embodiments, L is a heteroalkylene having from 4 to 30 atoms selected from carbon and oxygen. In certain embodiments, L is a heteroalkylene having from 4 to 20 atoms selected from carbon and oxygen. In certain embodiments, L is a heteroalkylene having from 4 to 10 atoms selected from carbon and oxygen.

In additional embodiments, the L is an optionally substituted (poly)ethyleneglycol having between 1 and about 100 ethylene glycol units, between about 1 and about 50 ethylene glycol units, between 1 and about 25 ethylene glycol units, between about 1 and about 10 ethylene glycol units, between 1 and about 8 ethylene glycol units, between 1 and about 6 ethylene glycol units, between 2 and about 4 ethylene glycol units, or optionally substituted alkyl groups interdispersed with optionally substituted, O, N, S, P or Si atoms. In certain embodiments, L is substituted with an aryl, phenyl, benzyl, alkyl, alkylene, or heterocycle group.

In certain embodiments, L is a bivalent, saturated or unsaturated, straight or branched C₁₋₄₅ hydrocarbon chain, wherein 0-10 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted carbocyclyl, or optionally substituted heterocyclyl, wherein R** represents independently for each occurrence hydrogen, C₁₋₆ alkyl, or C₃₋₆ cycloalkyl.

In certain embodiments, L is a bivalent, saturated or unsaturated, straight or branched C₁₋₄₅ hydrocarbon chain, wherein 0-10 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted 3-10 membered carbocyclyl, or optionally substituted 3-10 membered heterocyclyl containing 1, 2, 3, or 4 heteroatoms selected from nitrogen, oxygen, and sulfur, wherein R** represents independently for each occurrence hydrogen, C₁₋₆ alkyl, or C₃₋₆ cycloalkyl.

In certain embodiments, L has the formula —N(R)-(optionally substituted 3-20 membered heteroalkylene)ₚ-CH₂—C(O)—, wherein R is hydrogen or optionally substituted C₁-C₆ alkyl, and p is 0 or 1.

In certain embodiments, L has the formula —N(R)-(3-20 membered heteroalkylene)ₚ-CH₂—C(O)—; wherein the 3-20 membered heteroalkylene is optionally substituted with 1, 2, 3, or 4 substituents independently selected from halogen, C₁-C₆ haloalkyl, C₃-C₆ cycloalkyl, hydroxyl, and cyano; R is hydrogen or optionally substituted C₁-C₆ alkyl; and p is 0 or 1.

In certain embodiments, L has the formula —N(R)-(3-20 membered heteroalkylene)ₚ-CH₂—C(O)—; wherein the 3-20 membered heteroalkylene is optionally substituted with 1, 2, or 3 substituents independently selected from halogen and C₁-C₆ haloalkyl; R is hydrogen or C₁-C₆ alkyl; and p is 0 or 1.

In certain embodiments, L is —N(H)—(C₂₋₉ alkylene)-O—(C₁₋₆ alkylene)-C(O)—***, —N(H)—(C₁₀₋₂₀ alkylene)-O—(C₁₋₆ alkylene)-C(O)—***, —N(H)—[(C₂₋₄ alkylene)-O—]₂₋₆—(C₁₋₆ alkylene)-C(O)—***, —N(H)—[(C₂₋₄ alkylene)-O—]₇₋₁₅—(C₁₋₆ alkylene)-C(O)—***, —N(H)—(C₁₋₆ alkylene)-C(O)—***, —N(H)—(C₇₋₁₅ alkylene)-C(O)—***, —N(H)—[(C₂₋₄ alkylene)-O—]₂₋₆—(C₁₋₆ alkylene)-***, —N(H)—[(C₂₋₄ alkylene)-O—]₇₋₁₅—(C₁₋₆ alkylene)-***, —N(H)—(C₂₋₉ alkylene)-O—(C₁₋₆ alkylene)-C(O)N(C₁₋₆ alkyl)-(C₁₋₆ alkylene)-***, —N(H)—(C₂₋₉ alkylene)-O—(C₁₋₆ alkylene)-C(O)N(H)—(C₁₋₆ alkylene)-***, —N(H)—[(C₂₋₄ alkylene)-O—]₂₋₆—(C₁₋₆ alkylene)-N(H)—(C₁₋₆ alkylene)-***, —N(H)—[(C₂₋₄ alkylene)-O—]₇₋₁₅—(C₁₋₆ alkylene)-N(H)—(C₁₋₆ alkylene)-***, —N(H)—[(C₂₋₄ alkylene)-O—]₂₋₆—(C₁₋₆ alkylene)-N(C₁₋₆ alkyl)-(C₁₋₆ alkylene)-***, or —N(H)—[(C₂₋₄ alkylene)-O—]₇₋₁₅—(C₁₋₆ alkylene)-N(C₁₋₆ alkyl)-(C₁₋₆ alkylene)-***, where *** is a point of attachment to TPL.

In certain embodiments, L is —N(H)—(C₂₋₉ alkylene)-O—(C₁₋₆ alkylene)-C(O)—***, —N(H)—(C₁₀₋₂₀ alkylene)-O—(C₁₋₆ alkylene)-C(O)—***, —N(H)—[CH₂CH₂—O-]₂₋₆—(C₁₋₆ alkylene)-C(O)—***, —N(H)—[CH₂CH₂—O-]₇₋₁₅—(C₁₋₆ alkylene)-C(O)—***, —N(H)—(C₁₋₆ alkylene)-C(O)—***, —N(H)—(C₇₋₁₅ alkylene)-C(O)—***, —N(H)—[CH₂CH₂—O-]₂₋₆—(C₁₋₆ alkylene)-***, —N(H)—[CH₂CH₂-0-]₇₋₁₅—(C₁₋₆ alkylene)-***, —N(H)—(C₂₋₉ alkylene)-O—(C₁₋₆ alkylene)-C(O)N(C₁₋₆ alkyl)-(C₁₋₆ alkylene)-***, —N(H)—(C₂₋₉ alkylene)-O—(C₁₋₆ alkylene)-C(O)N(H)—(C₁₋₆ alkylene)-***, —N(H)—[CH₂CH₂—O-]₂₋₆—(C₁₋₆ alkylene)-N(H)—(C₁₋₆ alkylene)-***, —N(H)—[CH₂CH₂—O-]₇₋₁₅—(C₁₋₆ alkylene)-N(H)—(C₁₋₆ alkylene)-***, —N(H)—[CH₂CH₂—O-]₂₋₆—(C₁₋₆ alkylene)-N(C₁₋₆ alkyl)-(C₁₋₆ alkylene)-***, or —N(H)—[CH₂CH₂—O-]₇₋₁₅—(C₁₋₆ alkylene)-N(C₁₋₆ alkyl)-(C₁₋₆ alkylene)-***, where *** is a point of attachment to TPL.

In certain embodiments, L is —N(H)—[(C₂₋₄ alkylene)-O—]₂₋₆—(C₁₋₆ alkylene)-C(O)—***, —N(H)—[(C₂₋₄ alkylene)-O—]₇₋₁₅—(C₁₋₆ alkylene)-C(O)—***, —N(H)—(C₁₋₆ alkylene)-N(C₁₋₆ alkyl)C(O)—(C₁₋₆ alkylene)***, —N(H)—(C₁₋₆ alkylene)-N(H)C(O)—(C₁₋₆ alkylene)***, —N(H)—(C₂₋₆ alkylene)-***, —N(H)—(C₇₋₁₅ alkylene)-***, —N(C₁₋₆ alkyl)-(C₂₋₆ alkylene)-***, —N(C₁₋₆ alkyl)-(C₇₋₁₅ alkylene)-***, —N(H)—[(C₂₋₄ alkylene)-O—]₂₋₆—(C₁₋₆ alkylene)-***, —N(H)—[(C₂₋₄ alkylene)-O—]₇₋₁₅—(C₁₋₆ alkylene)-***, —N(H)—(C₁₋₆ alkylene)-(3-6 membered heterocycloalkylene)-(C₁₋₆ alkylene)-N(C₁₋₆ alkyl)-(C₁₋₆ alkylene)-***, —N(H)—(C₁₋₆ alkylene)-(3-6 membered heterocycloalkylene)-(C₁₋₆ alkylene)-N(H)—(C₁₋₆ alkylene)-***, —N(H)—(C₂₋₆ alkylene)-N(H)—(C₁₋₆ alkylene)-***, or —N(H)—(C₂₋₆ alkylene)-N(C₁₋₆ alkyl)-(C₁₋₆ alkylene)-***, where *** is a point of attachment to TPL.

In certain embodiments, L is —N(H)—[CH₂CH₂—O—]₂₋₆—(C₁₋₆ alkylene)-C(O)—***, —N(H)—[CH₂CH₂—O—]₇₋₁₅—(C₁₋₆ alkylene)-C(O)—***, —N(H)—(C₁₋₆ alkylene)-N(C₁₋₆ alkyl)C(O)—(C₁₋₆ alkylene)***, —N(H)—(C₁₋₆ alkylene)-N(H)C(O)—(C₁₋₆ alkylene)***, —N(H)—(C₂₋₆ alkylene)-***, —N(H)—(C₇₋₁₅ alkylene)-***, —N(C₁₋₆ alkyl)-(C₂₋₆ alkylene)-***, —N(C₁₋₆ alkyl)-(C₇₋₁₅ alkylene)-***, —N(H)—[CH₂CH₂—O-]₂₋₆—(C₁₋₆ alkylene)-***, —N(H)—[CH₂CH₂—O-]₇₋₁₅—(C₁₋₆ alkylene)-***, —N(H)—(C₁₋₆ alkylene)-(3-6 membered heterocycloalkylene)-(C₁₋₆ alkylene)-N(C₁₋₆ alkyl)-(C₁₋₆ alkylene)-***, —N(H)—(C₁₋₆ alkylene)-(3-6 membered heterocycloalkylene)-(C₁₋₆ alkylene)-N(H)—(C₁₋₆ alkylene)-***, —N(H)—(C₂₋₆ alkylene)-N(H)—(C₁₋₆ alkylene)-***, or —N(H)—(C₂₋₆ alkylene)-N(C₁₋₆ alkyl)-(C₁₋₆ alkylene)-***, where *** is a point of attachment to TPL.

In certain embodiments, L is —[(C₂₋₄ alkylene)-O—]₂₋₆—(C₁₋₆ alkylene)-***, —[(C₂₋₄ alkylene)-O—]₇₋₁₅—(C₁₋₆ alkylene)-***, —[(C₂₋₄ alkylene)-O—]₂₋₆—(C₁₋₆ alkylene)-N(C₁₋₆ alkyl)(C₁₋₆ alkylene)-***, —[(C₂₋₄ alkylene)-O—]₇-1₁—(C₁₋₆ alkylene)-N(C₁₋₆ alkyl)(C₁₋₆ alkylene)-***, —[(C₂₋₄ alkylene)-O—]₂₋₆—(C₁₋₆ alkylene)-N(H)(C₁₋₆ alkylene)-***, —[(C₂₋₄ alkylene)-O—]₇₋₁₅—(C₁₋₆ alkylene)-N(H)(C₁₋₆ alkylene)-***, —(C₁₋₉ alkylene)-C(O)N(H)—(C₁₋₆ alkylene)-***, —(C₁₋₉ alkylene)-N(H)C(O)—(C₁₋₆ alkylene)-***, —(C₁₋₉ alkylene)-C(O)N(H)—[(C₂₋₄ alkylene)-O—]₂₋₆—(C₁₋₆ alkylene)-***, —(C₁₋₉ alkylene)-N(H)C(O)—[(C₂₋₄ alkylene)-O—]₂₋₆—(C₁₋₆ alkylene)-***, —(C₁₋₉ alkylene)-C(O)N(H)—[(C₂₋₄ alkylene)-O—]₇₋₁₅—(C₁₋₆ alkylene)-***, —(C₁₋₉ alkylene)-N(H)C(O)—[(C₂₋₄ alkylene)-O—]₇₋₁₅—(C₁₋₆ alkylene)-***, —(C₁-9 alkylene)-C(O)N(H)—[(C₂₋₄ alkylene)-O—]2-6-(C₁₋₆ alkylene)-N(C₁₋₆ alkyl)-(C₁₋₆ alkylene)-***, —(C₁₋₉ alkylene)-N(H)C(O)—[(C₂₋₄ alkylene)-O-]₂₋₆—(C₁₋₆ alkylene)-N(C₁₋₆ alkyl)-(C₁₋₆ alkylene)-***, —(C₁-9 alkylene)-C(O)N(H)—[(C₂₋₄ alkylene)-O—]₇₋₁₅—(C₁₋₆ alkylene)-N(C₁₋₆ alkyl)-(C₁₋₆ alkylene)-***, or —(C₁₋₉ alkylene)-N(H)C(O)—[(C₂₋₄ alkylene)-O—]₇₋₁₅—(C₁₋₆ alkylene)-N(C₁₋₆ alkyl)-(C₁₋₆ alkylene)-***, where *** is a point of attachment to TPL.

In certain embodiments, L is —[CH₂CH₂—O—]₂₋₆—(C₁₋₆ alkylene)-***, —[CH₂CH₂—O—]₇₋₁₅—(C₁₋₆ alkylene)-***, —[CH₂CH₂—O—]₂₋₆—(C₁₋₆ alkylene)-N(C₁₋₆ alkyl)(C₁₋₆ alkylene)-***, —[CH₂CH₂—O—]₇₋₁₅—(C₁₋₆ alkylene)-N(C₁₋₆ alkyl)(C₁₋₆ alkylene)-***, —[CH₂CH₂—O—]₂₋₆—(C₁₋₆ alkylene)-N(H)(C₁₋₆ alkylene)-***, —[CH₂CH₂—O—]₇₋₁₅—(C₁₋₆ alkylene)-N(H)(C₁₋₆ alkylene)-***, —(C₁₋₉ alkylene)-C(O)N(H)—(C₁₋₆ alkylene)-***, —(C₁₋₉ alkylene)-N(H)C(O)—(C₁₋₆ alkylene)-***, —(C₁₋₉ alkylene)-C(O)N(H)—[CH₂CH₂—O—]₂₋₆—(C₁₋₆ alkylene)-***, —(C₁₋₉ alkylene)-N(H)C(O)—[CH₂CH₂—O—]₂₋₆—(C₁₋₆ alkylene)-***, —(C₁₋₉ alkylene)-C(O)N(H)—[CH₂CH₂—O—]₇₋₁₅—(C₁₋₆ alkylene)-***, —(C₁₋₉ alkylene)-N(H)C(O)—[CH₂CH₂—O—]₇₋₁₅—(C₁₋₆ alkylene)-***, —(C₁₋₉ alkylene)-C(O)N(H)—[CH₂CH₂—O—]₂₋₆—(C₁₋₆ alkylene)-N(C₁₋₆ alkyl)-(C₁₋₆ alkylene)-***, —(C₁₋₉ alkylene)-N(H)C(O)—[CH₂CH₂—O—]₂₋₆—(C₁₋₆ alkylene)-N(C₁₋₆ alkyl)-(C₁₋₆ alkylene)-***, —(C₁₋₉ alkylene)-C(O)N(H)—[CH₂CH₂—O—]₇₋₁₅—(C₁₋₆ alkylene)-N(C₁₋₆ alkyl)-(C₁₋₆ alkylene)-***, or —(C₁₋₉ alkylene)-N(H)C(O)—[(CH₂CH₂—O—]₇₋₁₅—(C₁₋₆ alkylene)-N(C₁₋₆ alkyl)-(C₁₋₆ alkylene)-***, where *** is a point of attachment to TPL.

In certain embodiments, L is —N(H)—[(C₂₋₄ alkylene)-O—]₂₋₆—(C₁₋₆ alkylene)-N(H)—***, —N(H)—[(C₂₋₄ alkylene)-O—]₇₋₁₅—(C₁₋₆ alkylene)-N(H)—***, —N(C₁₋₆ alkyl)-[(C₂₋₄ alkylene)-O—]₂₋₆—(C₁₋₆ alkylene)-N(H)—***, —N(C₁₋₆alkyl)-[(C₂₋₄ alkylene)-O—]₇₋₁₅—(C₁₋₆ alkylene)-N(H)—***, —N(C₁₋₆ alkyl)-[(C₂₋₄ alkylene)-O—]₂₋₆—(C₁₋₆ alkylene)-N(C₁₋₆ alkyl)-***, or —N(C₁₋₆ alkyl)-[(C₂₋₄ alkylene)-O—]₇₋₁₅—(C₁₋₆ alkylene)-N(C₁₋₆ alkyl)-***, where *** is a point of attachment to TPL.

In certain embodiments, L is —N(H)—[CH₂CH₂—O—]₂₋₆—(C₁₋₆ alkylene)-N(H)—***, —N(H)—[CH₂CH₂—O—]₇₋₁₅—(C₁₋₆ alkylene)-N(H)—***, —N(C₁₋₆ alkyl)-[CH₂CH₂—O—]₂₋₆—(C₁₋₆ alkylene)-N(H)—***, —N(C₁₋₆ alkyl)-[CH₂CH₂—O—]₇₋₁₅—(C₁₋₆ alkylene)-N(H)—***, —N(C₁₋₆ alkyl)-[CH₂CH₂—O—]₂₋₆—(C₁₋₆ alkylene)-N(C₁₋₆ alkyl)-***, or —N(C₁₋₆ alkyl)-[CH₂CH₂—O—]₇₋₁₅—(C₁₋₆ alkylene)-N(C₁₋₆ alkyl)-***, where *** is a point of attachment to TPL.

In some embodiments, L is one of the following:

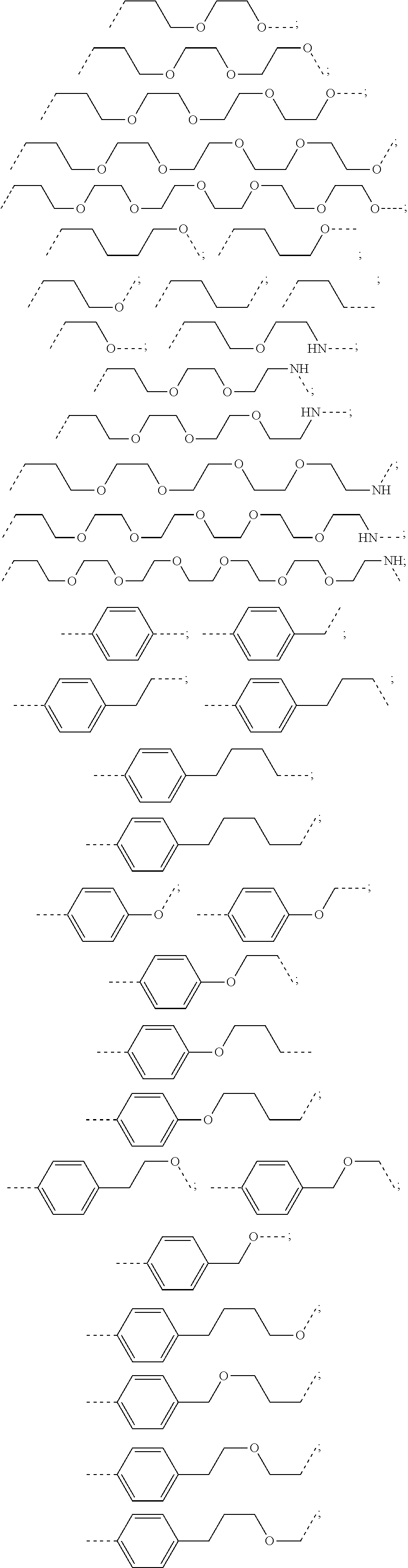

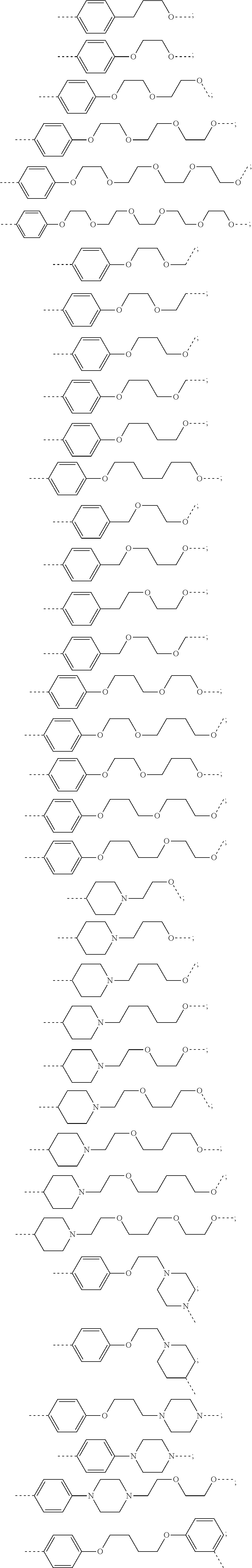

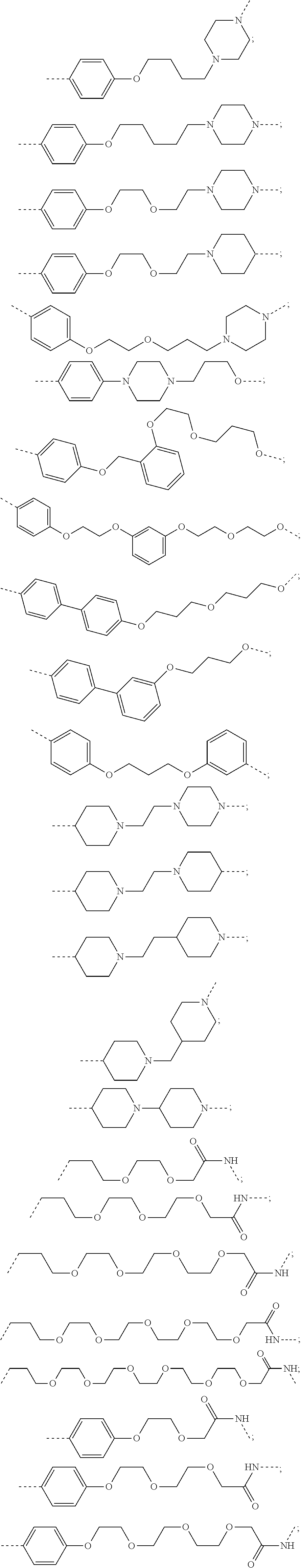

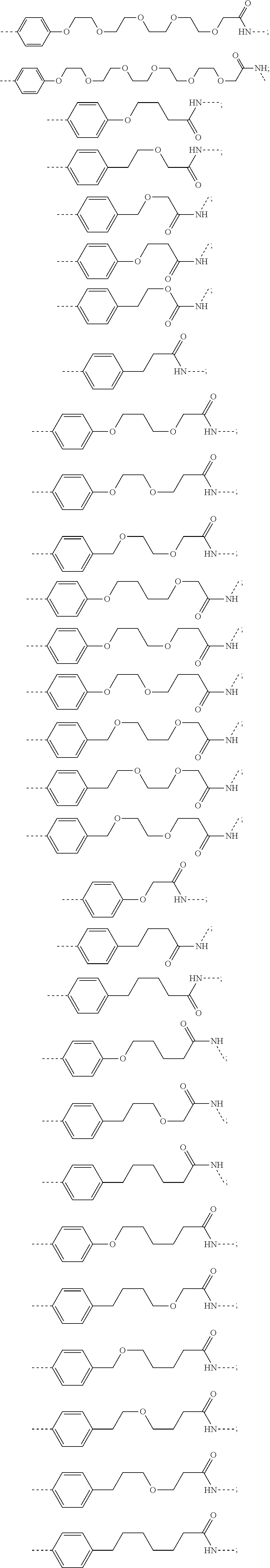

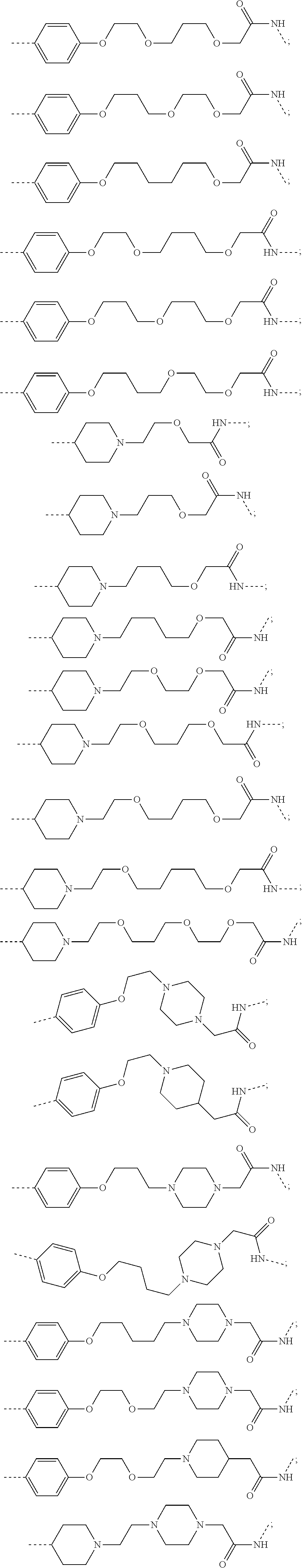

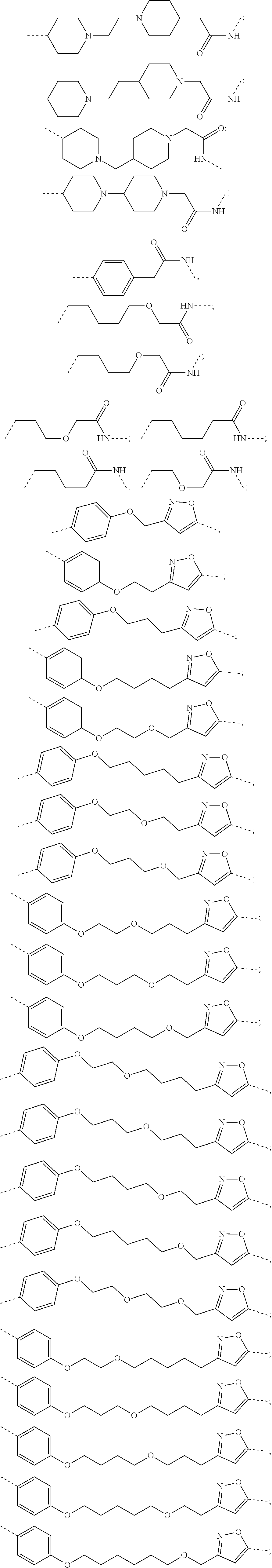

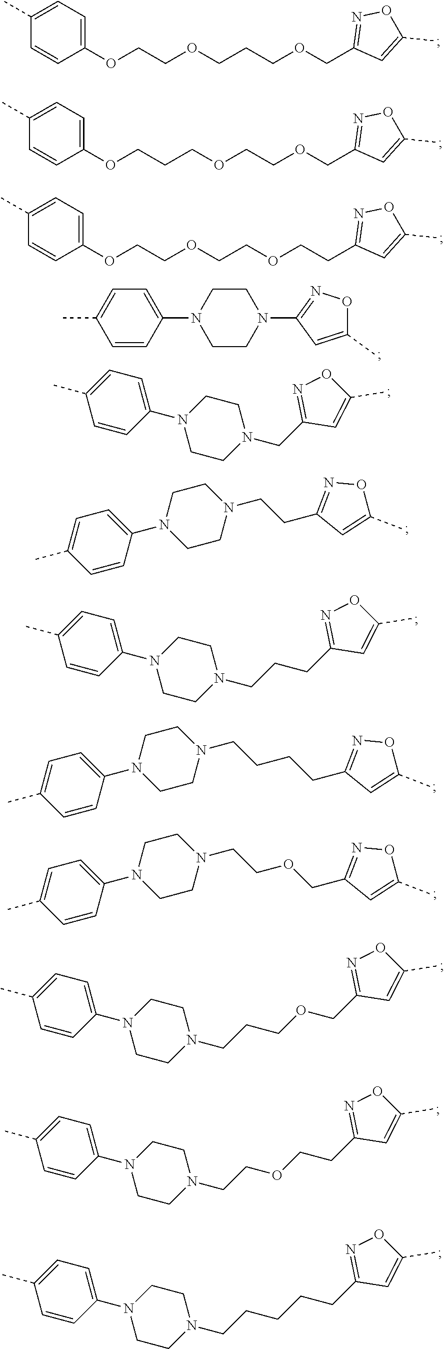

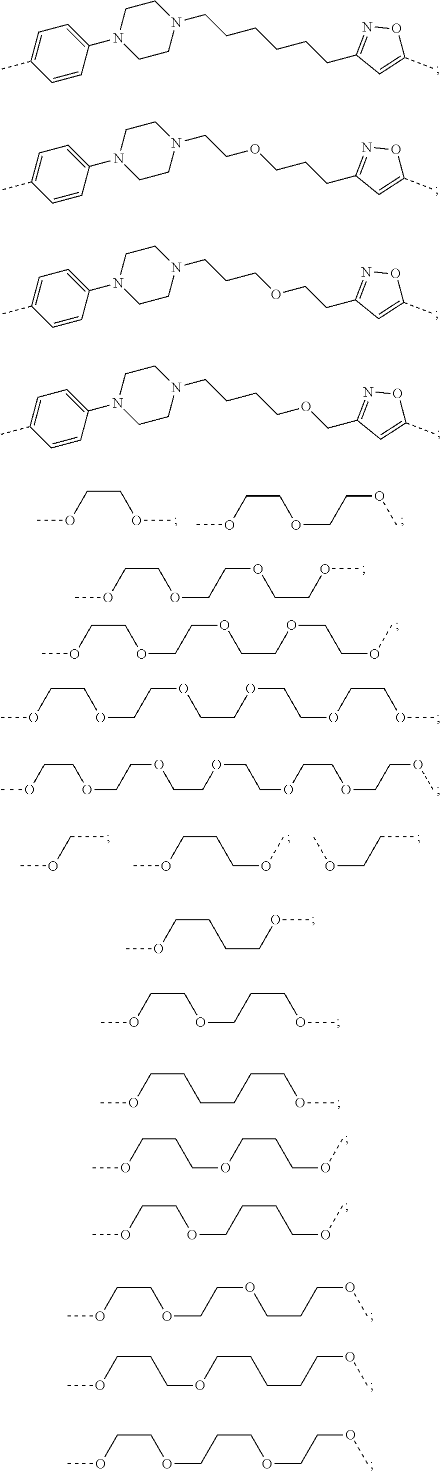

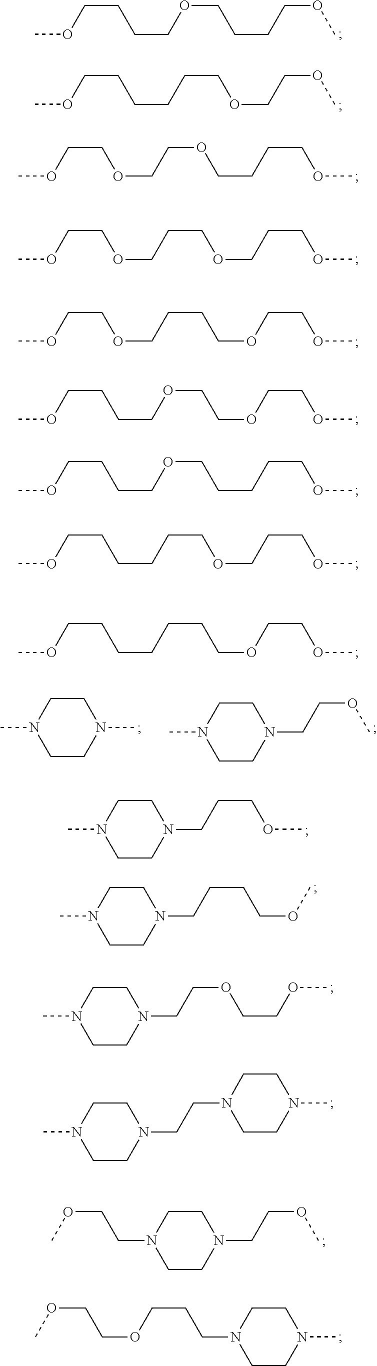

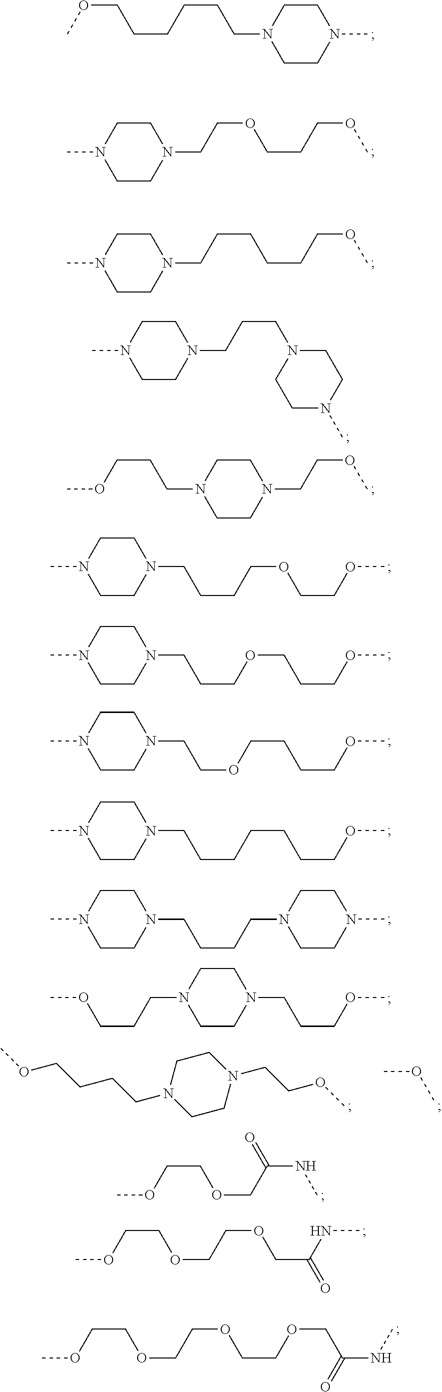

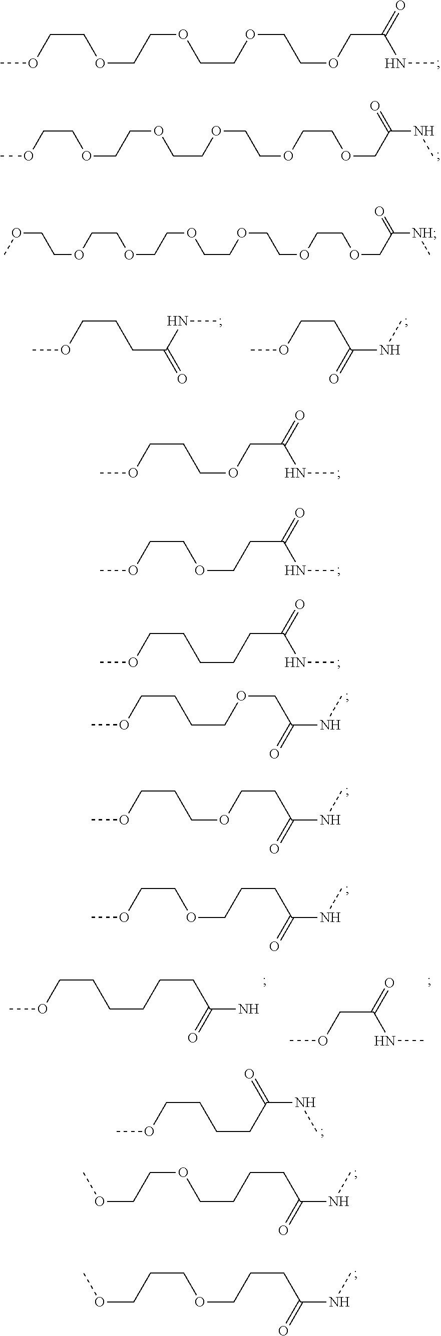

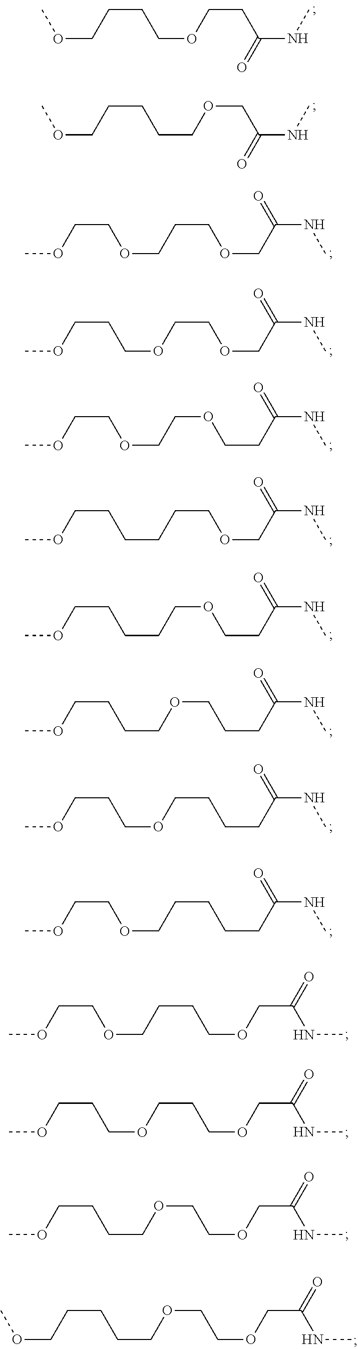

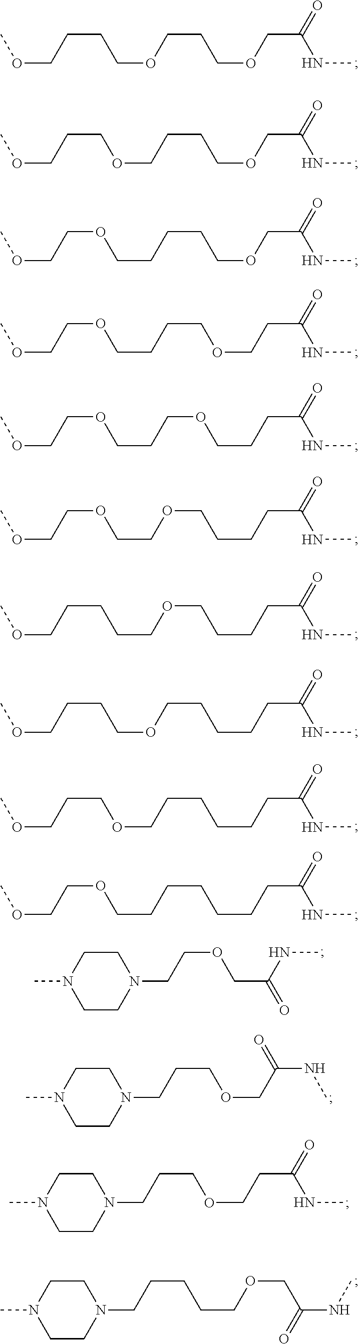

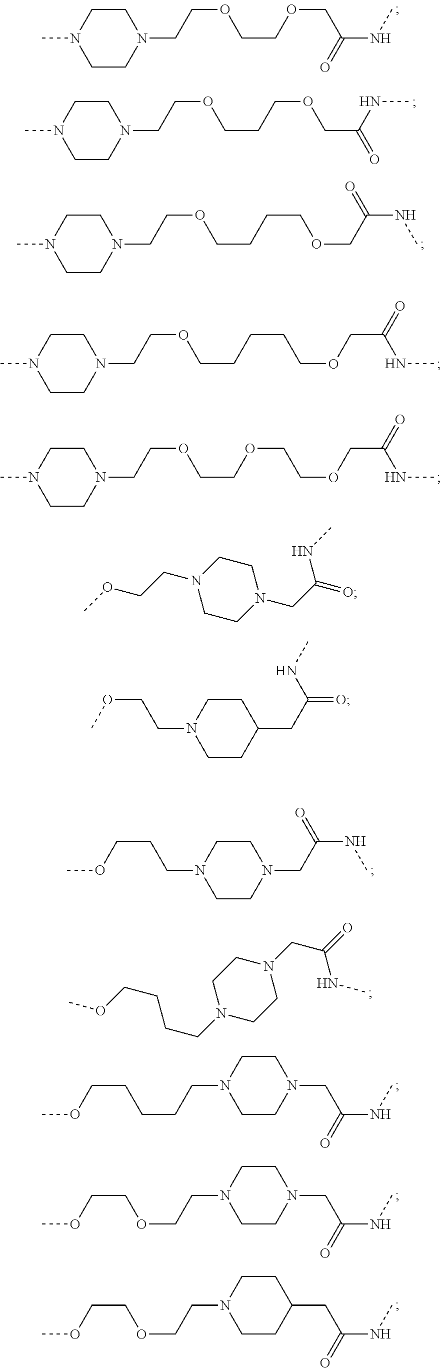

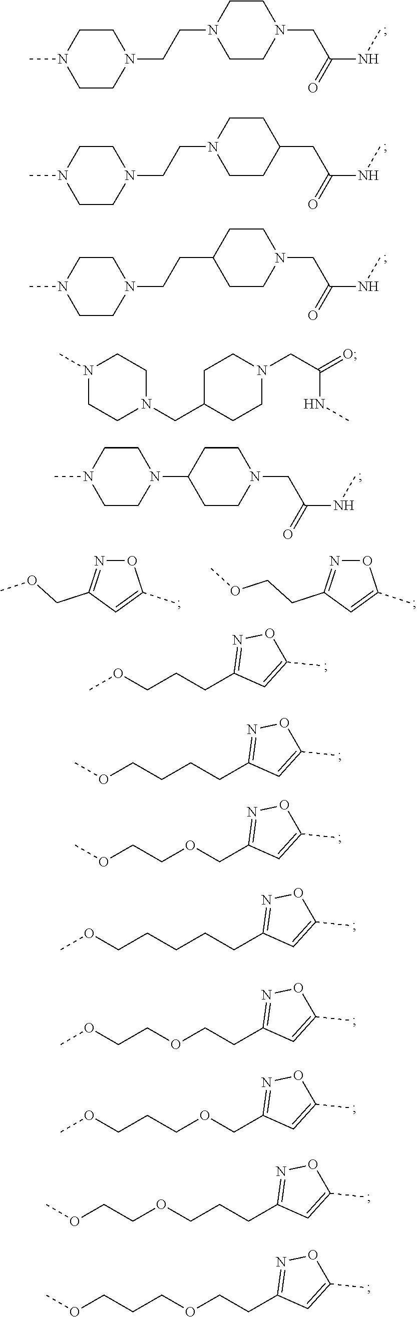

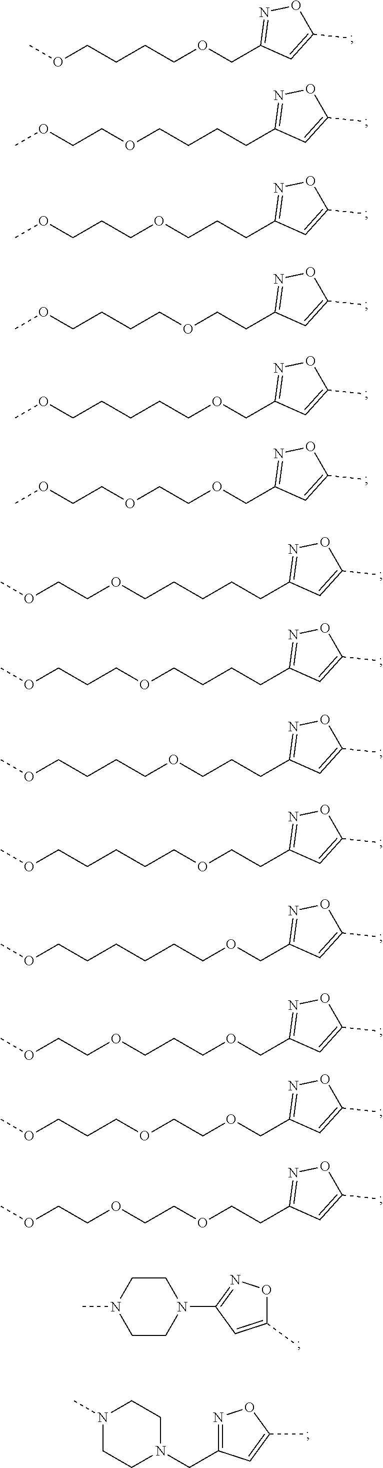

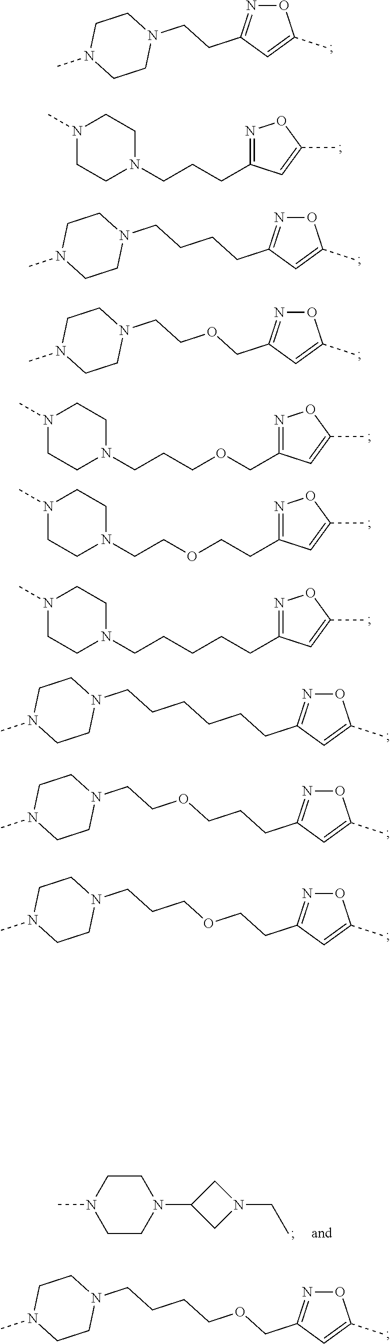

wherein a dashed bond indicates a point of attachment.

In certain embodiments, L has the formula —(C₀₋₁₂ alkylene)-(optionally substituted 3-40 membered heteroalkylene)-(C₀₋₁₂ alkylene)-. In certain embodiments, L is C₄₋₁₄ alkylene. In certain embodiments, L is —(CH₂)₆₋₁₀—

In certain embodiments, L is —Ch₂CH₂(OCH₂CH₂)—***, —CH₂CH₂(OCH₂CH₂)₂—***, —CH₂CH₂(OCH₂CH₂)₃—***, —CH₂CH₂(OCH₂CH₂)₄***, —CH₂CH₂(OCH₂CH₂)₅—***, —CH₂CH₂(OCH₂CH₂)₆—***, —CH₂CH₂(OCH₂CH₂)₇—***, —CH₂CH₂(OCH₂CH₂)₈—***, —CH₂CH₂(OCH₂CH₂)₉—***, —CH₂CH₂(OCH₂CH₂)₁₀—***, —CH₂CH₂(OCH₂CH₂)₁₁—***, —CH₂CH₂(OCH₂CH₂)₁₂—***, —CH₂CH₂(OCH₂CH₂)₁₃—***, —CH₂CH₂(OCH₂CH₂)₁₄—***, —CH₂CH₂(OCH₂CH₂)₁₅—***, or —CH₂CH₂(OCH₂CH₂)₁₆₋₂₀—***, where *** is a point of attachment to TPL.

In certain embodiments, L is —(C₂₋₂₀ alkylene)-(OCH₂CH₂)₂₋₄—(C₀₋₄ alkylene)-***, —(C₂₋₂₀ alkylene)-(OCH₂CH₂)₅₋₇—(C₀₋₄ alkylene)-***, —(C₂₋₂₀ alkylene)-(OCH₂CH₂)₈₋₁₀—(C₀₋₄ alkylene)-***, —(C₂₋₂₀ alkylene)-(OCH₂CH₂)₁₁₋₁₃—(C₀₋₄ alkylene)-***, —(C₂₋₂₀ alkylene)-(OCH₂CH₂)₁₄₋₁₆—(C₀₋₄ alkylene)-***, —(C₂₋₂₀ alkylene)-(OCH₂CH₂)₁₇₋₂₀—(C₀₋₄ alkylene)-***, —(C₁₋₂₀ alkylene)-(OCH₂CH₂)₁₋₁₀—(C₀₋₄ alkylene)-C(O)—***, or —(C₁₋₂₀ alkylene)-(OCH₂CH₂)₁₁₋₂₀—(C₀₋₄ alkylene)-C(O)—***, where *** is a point of attachment to TPL.

In certain embodiments, L is —O(CH₂CH₂O)₂₋₄—(C₀₋₄ alkylene)-***, —O(CH₂CH₂O)₅₋₇—(C₀₋₄ alkylene)-***, —O(CH₂CH₂O)₈₋₁₀—(C₀₋₄ alkylene)-***, —O(CH₂CH₂O)₁₁₋₁₃—(C₀₋₄ alkylene)-***, —O(CH₂CH₂O)₁₄₋₁₆—(C₀₋₄ alkylene)-***, —O(CH₂CH₂O)₁₆₋₂₀—(C₀₋₄ alkylene)-***, —O(CH₂CH₂O)₂₋₁₀—(C₀₋₄ alkylene)C(O)—***, or —O(CH₂CH₂O)₁₁₋₂₀—(C₀₋₄ alkylene)C(O)—***, where *** is a point of attachment to TPL.

In certain embodiments, L is —(C₀₋₂₀ alkylene)-(OCH₂CH₂)₁₋₁₀—(N(C₁₋₄ alkyl))-***, —(C₀₋₂₀ alkylene)-(OCH₂CH₂)₁₋₂₀—(N(C₁₋₄ alkyl))-***, —(C₀₋₂₀ alkylene)-(CH₂CH₂O)₁₋₁₀—(C₂₋₁₀ alkylene)-(N(C₁₋₄ alkyl))—(C₀₋₁₀ alkylene)-***, or —(C₀₋₂₀ alkylene)-(CH₂CH₂O)₁₁₋₂₀—(C₂₋₁₀ alkylene)-(N(C₁₋₄ alkyl))-(C₀₋₁₀ alkylene)-***, where *** is a point of attachment to TPL.

In certain embodiments, L is —(C₂₋₁₀ alkylene)-(OCH₂CH₂)₂₋₄—O-(3-6 membered saturated heterocyclylene containing 1 or 2 heteroatoms independently selected from nitrogen and oxygen)-(C₁₋₅ alkylene)-***, —(C₂₋₁₀ alkylene)-(3-6 membered saturated heterocyclylene containing 1 or 2 heteroatoms independently selected from nitrogen and oxygen)-(C₁₋₅ alkylene)-***, —(C₂₋₁₀ alkylene)-N(H)—(C₁₋₅alkylene)-***, —(C₂₋₁₀ alkylene)-N(C₁₋₆ alkyl)-(C₁₋₅ alkylene)-***, —N(H)—(C₁₋₅ alkylene)-***, —(CH₂CH₂O)₁₋₄—(C₁₋₄ alkylene)-***, —(CH₂CH₂O)₁₋₄—(C₁₋₄ alkylene)-N(C₁₋₆ alkyl)-(C₁₋₆ alkylene)-***, —N(H)—(C₂₋₆ alkylene)-(3-6 membered saturated heterocyclylene containing 1 or 2 heteroatoms independently selected from nitrogen and oxygen)-(C₁₋₅alkylene)-***, —N(H)—(C₂₋₆ alkylene)-(OCH₂CH₂)₁₋₄—O-(3-6 membered saturated heterocyclylene containing 1 or 2 heteroatoms independently selected from nitrogen and oxygen)-(C₁₋₅alkylene)-***, —N(H)—[—CH₂CH₂O—]₂₋₆—(C₁₋₆ alkylene)-N(C₁₋₆ alkyl)-(C₁₋₆ alkylene)-***, —(C₁₋₆ alkylene)-(3-6 membered saturated heterocyclylene containing 1 or 2 heteroatoms independently selected from nitrogen and oxygen)-(C₁₋₆ alkylene)-***, —N(H)—(C₂-10 alkylene)-***, —(C₁₋₆ alkylene)-(3-6 membered saturated heterocyclylene containing 1 or 2 heteroatoms independently selected from nitrogen and oxygen)-(C₁₋₆ alkylene)-N(C₁₋₆ alkyl)-(C₁₋₆ alkylene)-***, —N(H)—[CH₂CH₂—O—]₂₋₆—(C₁₋₆ alkylene)-N(C₁₋₆ alkyl)-(C₁₋₆ alkylene)-***, —N(H)—[CH₂CH₂—O—]₂₋₁₀—(C₁₋₆ alkylene)-***, —N(H)—[CH₂CH₂—O—]₂₋₆—(C₁₋₆ alkylene)-N(C₁₋₆ alkyl)C(O)—(C₁₋₆ alkylene)-***, —[CH₂CH₂—O—]₁₋₆—(C₁₋₆ alkylene)-N(C₁₋₆ alkyl)-***, —[CH₂CH₂—O—]₁₋₆—(C₁₋₆ alkylene)-N(H)—***, or —(C₂₋₁₀ alkylene)-(OCH₂CH₂)₂₋₆—(C₃₋₆ cycloalkylene)-***, where *** is a point of attachment to TPL.

In certain embodiments, L is selected from those depicted in the compounds in Table 3, below.

### Exemplary Specific Compounds

In certain embodiments, the compound is a compound in Table 1, or a pharmaceutically acceptable salt thereof. In certain embodiments, the compound is a compound in Table 1. In certain embodiments, the compound is a compound in Table 1-A, or a pharmaceutically acceptable salt thereof. In certain embodiments, the compound is a compound in Table 1-A.

TABLE 1
Compound No. from Working Examples

I-1
I-2
I-3
I-4
I-5
I-6
I-7
I-8
I-9
I-10
I-11
I-12
I-13
I-14
I-15
I-16
I-17
I-18
I-19
I-20
I-21
I-22
I-23
I-24
I-25
I-26
I-27
I-28
I-29
I-30
I-31
I-32
I-33
I-34
I-35
I-36
I-37
I-38
I-39
I-40
I-41
I-42
I-43
I-44
I-45
I-46
I-47
I-48
I-49
I-50
I-51
I-52
I-53
I-54
I-55
I-56
I-57
I-58
I-59
I-60
I-61
I-62
I-63
I-64
I-65
I-66
I-67
I-68
I-69
I-70
I-71
I-72
I-73
I-74
I-75
I-76
I-77
I-78
I-79
I-80
I-81
I-82

TABLE 1-A
Compound No. from Working Examples

I-83
I-84
I-85
I-86
I-87
I-88
I-89
I-90
I-91
I-92
I-93
I-94
I-95
I-96
I-97
I-98
I-99
I-100
I-101
I-102
I-103
I-104
I-105
I-106
I-107
I-108
I-109
I-110
I-111
I-112
I-113
I-114
I-115
I-116
I-117
I-118
I-119
I-120
I-121
I-122
I-123
I-124
I-125
I-126
I-127
I-128
I-129
I-130
I-131
I-132
I-133
I-134
I-135
I-136
I-137
I-138
I-139
I-140
I-141
I-142
I-143
I-144
I-145
I-146
I-147
I-148
I-149
I-150
I-151
I-152

### Synthetic Methods

Methods for preparing compounds described herein are illustrated in the following synthetic Schemes. The Schemes are given for the purpose of illustrating the invention, and are not intended to limit the scope or spirit of the invention. Starting materials shown in the Schemes can be obtained from commercial sources or can be prepared based on procedures described in the literature.

In the Schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in “Protecting Groups in Organic Synthesis”, T. W. Greene and P. G. M. Wuts, 3^{rd} edition, John Wiley & Sons, 1999, the entire contents of which are hereby incorporated by reference.

The synthetic route illustrated in Scheme 1 is a general method for preparing heterobifunctional compounds D. Coupling compound A (a precursor of TPL, for example, a discrete compound that is a target protein ligand) with L′ (a precursor to linker L, containing functionality for coupling to the precursors of both TPL and EPL) affords intermediate B (wherein L is a precursor to linker L that contains functionality for coupling to the EPL precursor). Coupling intermediate B with compound C (a precursor of EPL) affords heterobifunctional compound D. Alternatively, the order of coupling compounds A and C to L′ may be reversed, such that L′ is first coupled with compound C, before being coupled to compound A.

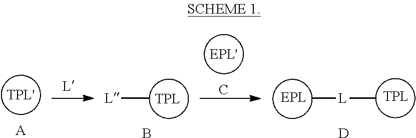

The coupling of compound A with L′, and the coupling of intermediate B with compound C, can be accomplished with a wide variety of strategies. For example, amide coupling conditions can be employed when compound A (or compound C) is to be attached at a modifiable nitrogen atom and L′ (or L) contains a carboxylic acid group, or vice versa (i.e. compound A contains a carboxylic acid group and L′ contains a nucleophilic amine nitrogen atom). Alternatively, reductive amination conditions can be employed when compound A (or compound C) is to be attached at a modifiable nitrogen atom and L′ (or L) contains an aldehyde group, or vice versa. Alternatively, nucleophilic substitution conditions can be employed when compound A (or compound C) is to be attached at a modifiable oxygen, nitrogen, or sulfur atom and L′ (or L″) contains a leaving group (such as an alkyl triflate, α-bromoketone, or aryl chloride), or vice versa. As yet another option, transition-metal-mediated coupling conditions can be employed when compound A (or compound C) is to be attached at a modifiable carbon, oxygen, or nitrogen atom (where the carbon atom may be activated, for example, with a bromide or sulfonate) and L′ (or L″) contains a suitable coupling partner (for example, an olefin for a Heck coupling, a trialkylstannane for a Stille coupling, or a boronic acid or boronate ester for a Suzuki coupling, Buchwald-Hartwig amination, or Chan-Lam coupling), or vice versa.

It is understood by one skilled in the art of organic synthesis that protecting group strategies may be employed as necessary, for example, if L′ contains two of the same functional group that are to be selectively coupled to compound A and compound C. For example, L′ may contain, for example, both an unprotected carboxylic acid for coupling to compound A, and a carboxylic acid group that is protected (for example, as a methyl or benzyl ester) during the coupling with compound A and subsequently deprotected (for example, via basic hydrolysis of a methyl ester or hydrogenolysis of a benzyl ester) prior to coupling with compound C.

### II. Therapeutic Applications

The heterobifunctional compounds described herein, such as a compound of Formula I, or other compounds in Section I, provide therapeutic benefits to patients suffering from cancer and/or hepatitis. Accordingly, one aspect of the invention provides a method of treating cancer. The method comprises administering to a patient in need thereof a therapeutically effective amount of a compound described herein, such as a compound of Formula I, or other compounds in Section I, to treat the cancer. In certain embodiments, the particular compound of Formula I is a compound defined by one of the embodiments described above.

Another aspect of the invention provides a method of treating hepatitis. The method comprises administering to a patient in need thereof a therapeutically effective amount of a compound described herein, such as a compound of Formula I, or other compounds in Section I, to treat the hepatitis. In certain embodiments, the particular compound of Formula I is a compound defined by one of the embodiments described above.

### Cancer

In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia.

In certain embodiments, the cancer is squamous cell cancer, lung cancer including small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer. In certain embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma myeloproliferative diseases, large B cell lymphoma, or B cell Lymphoma.

In certain embodiments, the cancer is a solid tumor or leukemia. In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, espophagus cancer, liver cancer, an acoustic neuroma, oligodendroglioma, meningioma, neuroblastoma, or retinoblastoma. In certain other embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain other embodiments, the cancer is colon cancer, small-cell lung cancer, non-small cell lung cancer, renal cancer, ovarian cancer, renal cancer, or melanoma.

In certain embodiments, the cancer is a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, or hemangioblastoma.

In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In certain embodiments, the cancer is hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In certain embodiments, the cancer is hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is a solid tumor, such as a sarcoma, carcinoma, or lymphoma. In certain embodiments, the cancer is kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In certain embodiments, the cancer is renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

### Hepatitis

In certain embodiments, the disease to be treated is hepatitis. In certain embodiments, the hepatitis is hepatitis A, B, or C.

### Causing Death of Cancer Cell

Another aspect of the invention provides a method of causing death of a cancer cell. The method comprises contacting a cancer cell with an effective amount of a compound described herein, such as a compound of Formula I or II, or other compounds in Section I, to cause death of the cancer cell. In certain embodiments, the particular compound of Formula I or II is a compound defined by one of the embodiments described above.

In certain embodiments, the cancer cell is selected from ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia. In certain embodiments, the cancer cell is one or more of the cancers recited in the section above entitled “Cancer.”

### Combination Therapies

The compounds useful within the methods of the invention may be used in combination with one or more additional therapeutic agents useful for treating any disease contemplated herein. These additional therapeutic agents may comprise compounds that are commercially available or synthetically accessible to those skilled in the art. These additional therapeutic agents are known to treat, prevent, or reduce the symptoms, of a disease or disorder contemplated herein.

Accordingly, in certain embodiments, the method further comprises administering to the subject an additional therapeutic agent that treats the disease contemplated herein.

In certain embodiments, administering the compound of the invention to the subject allows for administering a lower dose of the additional therapeutic agent as compared to the dose of the additional therapeutic agent alone that is required to achieve similar results in treating the disease contemplated herein. For example, in certain embodiments, the compound of the invention enhances the therapeutic activity of the additional therapeutic compound, thereby allowing for a lower dose of the additional therapeutic compound to provide the same effect.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In certain embodiments, the compound of the invention and the therapeutic agent are co-administered to the subject. In other embodiments, the compound of the invention and the therapeutic agent are coformulated and co-administered to the subject.

In certain embodiments, the compound is administered in combination with a second therapeutic agent having activity against cancer. In certain embodiments, the second therapeutic agent is mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

In certain embodiments, the second therapeutic agent is an mTOR inhibitor, which inhibits cell proliferation, angiogenesis and glucose uptake. Approved mTOR inhibitors useful in the present invention include everolimus (Afinitor®, Novartis); temsirolimus (Torisel®, Pfizer); and sirolimus (Rapamune®, Pfizer).

In certain embodiments, the second therapeutic agent is a Poly ADP ribose polymerase (PARP) inhibitor. Approved PARP inhibitors useful in the present invention include olaparib (Lynparza®, AstraZeneca); rucaparib (Rubraca®, Clovis Oncology); and niraparib (Zejula®, Tesaro). Other PARP inhibitors being studied which may be used in the present invention include talazoparib (MDV3800/BMN 673/LT00673, Medivation/Pfizer/Biomarin); veliparib (ABT-888, AbbVie); and BGB-290 (BeiGene, Inc.).

In certain embodiments, the second therapeutic agent is a phosphatidylinositol 3 kinase (PI3K) inhibitor. Approved PI3K inhibitors useful in the present invention include idelalisib (Zydelig®, Gilead). Other PI3K inhibitors being studied which may be used in the present invention include alpelisib (BYL719, Novartis); taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In certain embodiments, the second therapeutic agent is a proteasome inhibitor. Approved proteasome inhibitors useful in the present invention include bortezomib (Velcade®, Takeda); carfilzomib (Kyprolis®, Amgen); and ixazomib (Ninlaro®, Takeda).

In certain embodiments, the second therapeutic agent is a histone deacetylase (HDAC) inhibitor. Approved HDAC inhibitors useful in the present invention include vorinostat (Zolinza®, Merck); romidepsin (Istodax®, Celgene); panobinostat (Farydak®, Novartis); and belinostat (Beleodaq®, Spectrum Pharmaceuticals). Other HDAC inhibitors being studied which may be used in the present invention include entinostat (SNDX-275, Syndax Pharmaceuticals) (NCT00866333); and chidamide (Epidaza®, HBI-8000, Chipscreen Biosciences, China).

In certain embodiments, the second therapeutic agent is a CDK inhibitor, such as a CDK 4/6 inhibitor. Approved CDK 4/6 inhibitors useful in the present invention include palbociclib (Ibrance®, Pfizer); and ribociclib (Kisqali®, Novartis). Other CDK 4/6 inhibitors being studied which may be used in the present invention include abemaciclib (Ly2835219, Eli Lilly); and trilaciclib (G1T28, G1 Therapeutics).

In certain embodiments, the second therapeutic agent is an indoleamine (2,3)-dioxygenase (IDO) inhibitor. IDO inhibitors being studied which may be used in the present invention include epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); and an enzyme that breaks down kynurenine (Kynase, Kyn Therapeutics).

In certain embodiments, the second therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

In certain embodiments, the second therapeutic agent is an aromatase inhibitor. Approved aromatase inhibitors which may be used in the present invention include exemestane (Aromasin®, Pfizer); anastazole (Arimidex®, AstraZeneca) and letrozole (Femara®, Novartis).

In certain embodiments, the second therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In certain embodiments, the second therapeutic agent is a folic acid inhibitor. Approved folic acid inhibitors useful in the present invention include pemetrexed (Alimta®, Eli Lilly).

In certain embodiments, the second therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In certain embodiments, the second therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In certain embodiments, the second therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In certain embodiments, the second therapeutic agent is a glutaminase inhibitor. Glutaminase inhibitors being studied which may be used in the present invention include CB-839 (Calithera Biosciences).

In certain embodiments, the second therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin@, Spectrum Pharmaceuticals); daratumumab (anti-CD38, Darzalex@, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla@, Genentech); and pertuzumab (anti-HER2, Perjeta®, Genentech); and brentuximab vedotin (anti-CD30-drug conjugate, Adcetris®, Seattle Genetics).

In certain embodiments, the second therapeutic agent is a topoisomerase inhibitor. Approved topoisomerase inhibitors useful in the present invention include irinotecan (Onivyde@, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In certain embodiments, the second therapeutic agent is a nucleoside inhibitor, or other therapeutic that interfere with normal DNA synthesis, protein synthesis, cell replication, or will otherwise inhibit rapidly proliferating cells. Such nucleoside inhibitors or other therapeutics include trabectedin (guanidine alkylating agent, Yondelis@, Janssen Oncology), mechlorethamine (alkylating agent, Valchlor@, Aktelion Pharmaceuticals); vincristine (Oncovin@, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine@, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo@; Teva Pharmaceuticals); asparaginase Erwinia chrysanthemi (enzyme for depletion of asparagine, Elspar@, Lundbeck; Erwinaze®, EUSA Pharma); eribulin mesylate (microtubule inhibitor, tubulin-based antimitotic, Halaven@, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In certain embodiments, the second therapeutic agent is a platinum-based therapeutic, also referred to as platins. Platins cause cross-linking of DNA, such that they inhibit DNA repair and/or DNA synthesis, mostly in rapidly reproducing cells, such as cancer cells. Approved platinum-based therapeutics which may be used in the present invention include cisplatin (Platinol®, Bristol-Myers Squibb); carboplatin (Paraplatin®, Bristol-Myers Squibb; also, Teva; Pfizer); oxaliplatin (Eloxitin® Sanofi-Aventis); and nedaplatin (Aqupla®, Shionogi). Other platinum-based therapeutics which have undergone clinical testing and may be used in the present invention include picoplatin (Poniard Pharmaceuticals); and satraplatin (JM-216, Agennix).

In certain embodiments, the second therapeutic agent is a taxane compound, which causes disruption of microtubules, which are essential for cell division. Approved taxane compounds which may be used in the present invention include paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), and cabazitaxel (Jevtana®, Sanofi-Aventis). Other taxane compounds which have undergone clinical testing and may be used in the present invention include SID530 (SK Chemicals, Co.) (NCT00931008).

In certain embodiments, the second therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In certain embodiments, the second therapeutic agent is a selective estrogen receptor modulator (SERM), which interferes with the synthesis or activity of estrogens. Approved SERMs useful in the present invention include raloxifene (Evista®, Eli Lilly).

In certain embodiments, the second therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In certain embodiments, the second therapeutic agent is an inhibitor of transforming growth factor-beta (TGF-beta or TGFβ). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA-formerly MSB0011459X), which is a bispecific, anti-PD-L1/TGFβ trap compound (NCT02699515); and (NCT02517398). M7824 is comprised of a fully human IgGI antibody against PD-L1 fused to the extracellular domain of human TGF-beta receptor II, which functions as a TGFβ “trap.”

In certain embodiments, the second therapeutic agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the additional therapeutic agent is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In certain embodiments, the second therapeutic agent is an immune checkpoint inhibitor selected from a PD-1 antagonist, a PD-L1 antagonist, or a CTLA-4 antagonist. In some embodiments, a compound disclosed herein or a pharmaceutically acceptable salt thereof is administered in combination with nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); or atezolizumab (anti-PD-L1 antibody, Tecentriq®, Genentech). Other immune checkpoint inhibitors suitable for use in the present invention include REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgGI anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; and PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

Another aspect of the invention provides for the use of a compound described herein (such as a compound of Formula I, Formula II, or other compounds in Section I) in the manufacture of a medicament. In certain embodiments, the medicament is for treating a disease described herein, such as cancer.

Another aspect of the invention provides for the use of a compound described herein (such as a compound of Formula I, Formula II, or other compounds in Section I) for treating a medical disease, such a disease described herein (e.g., cancer).

### III. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (e.g., a compound of Formula I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a heterobifunctional substituted phenylpyrimidinone or related compound described herein in a therapeutically effective amount for the treatment of a medical disorder described herein.

### IV. Medical Kits

Another aspect of this invention is a kit comprising (i) a compound described herein, such as a compound of Formula I, and (ii) instructions for use, such as treating cancer.

### V. ASSAYS

Activity of compounds described herein can be evaluated using the following assays.

### Part I—Assay for Binding Affinity to CDK9

Compounds may be tested for ability to bind to CDK9, according to the following experimental procedure.

Kinase-tagged T7 phage strains are prepared in an E. coli host derived from the BL21 strain. E. coli are grown to log-phase and infected with T7 phage and incubated with shaking at 32° C. until lysis. The lysates are centrifuged and filtered to remove cell debris. The remaining kinases are produced in HEK-293 cells and subsequently tagged with DNA for qPCR detection. Streptavidin-coated magnetic beads are treated with biotinylated small molecule ligands for 30 minutes at room temperature to generate affinity resins for kinase assays. The liganded beads are blocked with excess biotin and washed with blocking buffer (SeaBlock (Pierce), 1% BSA, 0.05% Tween 20, 1 mM DTT) to remove unbound ligand and to reduce non-specific binding. Binding reactions are assembled by combining kinases, liganded affinity beads, and test compounds in 1× binding buffer (20% SeaBlock, 0.17×PBS, 0.05% Tween 20, 6 mM DTT). Test compounds are prepared as 111X stocks in 100% DMSO. Kd values are determined using an 11-point 3-fold compound dilution series with three DMSO control points. All compounds for Kd measurements are distributed by acoustic transfer (non-contact dispensing) in 100% DMSO. The compounds are then diluted directly into the assays such that the final concentration of DMSO is 0.9%. All reactions are performed in polypropylene 384-well plates. Each has a final volume of 0.02 mL. The assay plates are incubated at room temperature with shaking for 1 hour and the affinity beads are washed with wash buffer (1× PBS, 0.05% Tween 20). The beads are then re-suspended in elution buffer (1× PBS, 0.05% Tween 20, 0.5 μM nonbiotinylated affinity ligand) and incubated at room temperature with shaking for 30 minutes. The kinase concentration in the eluates is measured by qPCR.

### Part II—Assay for Binding Affinity to PLK1

Compounds may be tested for ability to bind to PLK1, according to the following experimental procedure.

Kinase-tagged T7 phage strains are prepared in an E. coli host derived from the BL21 strain. E. coli are grown to log-phase and infected with T7 phage and incubated with shaking at 32° C. until lysis. The lysates are centrifuged and filtered to remove cell debris. The remaining kinases are produced in HEK-293 cells and subsequently tagged with DNA for qPCR detection. Streptavidin-coated magnetic beads are treated with biotinylated small molecule ligands for 30 minutes at room temperature to generate affinity resins for kinase assays. The liganded beads are blocked with excess biotin and washed with blocking buffer (SeaBlock (Pierce), 1% BSA, 0.05% Tween 20, 1 mM DTT) to remove unbound ligand and to reduce non-specific binding. Binding reactions are assembled by combining kinases, liganded affinity beads, and test compounds in 1× binding buffer (20% SeaBlock, 0.17×PBS, 0.05% Tween 20, 6 mM DTT). Test compounds are prepared as 111X stocks in 100% DMSO. Kd values are determined using an 11-point 3-fold compound dilution series with three DMSO control points. All compounds for Kd measurements are distributed by acoustic transfer (non-contact dispensing) in 100% DMSO. The compounds are then diluted directly into the assays such that the final concentration of DMSO is 0.9%. All reactions are performed in polypropylene 384-well plates. Each has a final volume of 0.02 mL. The assay plates are incubated at room temperature with shaking for 1 hour and the affinity beads are washed with wash buffer (1×PBS, 0.05% Tween 20). The beads are then re-suspended in elution buffer (1×PBS, 0.05% Tween 20, 0.5 μM nonbiotinylated affinity ligand) and incubated at room temperature with shaking for 30 minutes. The kinase concentration in the eluates is measured by qPCR.

### EXAMPLES

The invention now being generally described, will be more readily understood by reference to the following examples, which are included merely for purposes of illustration of certain aspects and embodiments of the present invention, and is not intended to limit the invention.

### General Methods

All reactions were carried out under an atmosphere of dry nitrogen or argon. Glassware was oven-dried prior to use. Unless otherwise indicated, common reagents or materials were obtained from commercial sources and used without further purification. N,N-Diisopropylethylamine (DIPEA) was obtained anhydrous by distillation over potassium hydroxide. Tetrahydrofuran (THF), Dichloromethane (CH₂Cl₂), and dimethylformamide (DMF) was dried by a PureSolv™ solvent drying system. PTLC refers to preparatory thin layer chromatographic separation. Abbreviations: HFIP (hexafluoroisopropanol), HEPES (4-(2-hydroxyethyl)-1-piperazineethanesulfonic acid. Flash column chromatography was performed using silica gel 60 (230-400 mesh). Analytical thin layer chromatography (TLC) was carried out on Merck silica gel plates with QF-254 indicator and visualized by UV or KMnO₄.

¹H and ¹³C NMR spectra were recorded on an Agilent DD₂ 500 (500 MHz ¹H; 125 MHz ¹³C) or Agilent DD₂ 600 (600 MHz ¹H; 150 MHz ¹³C) or Agilent DD₂ 400 (400 MHz ¹H; 100 MHz ¹³C) spectrometer at room temperature. Chemical shifts were reported in ppm relative to the residual CDCl₃ (δ 7.26 ppm ¹H; δ 77.0 ppm ¹³C), CD₃OD (δ 3.31 ppm ¹H; δ 49.00 ppm ¹³C), or d₆-DMSO (δ 2.50 ppm ¹H; δ 39.52 ppm ¹³C). NMR chemical shifts were expressed in ppm relative to internal solvent peaks, and coupling constants were measured in Hz. (bs=broad signal). In most cases, only peaks of the major rotamer are reported.

Mass spectra were obtained using Agilent 1100 series LC/MSD spectrometers. Analytical HPLC analyses were carried out on 250×4.6 mm C-18 column using gradient conditions (10-100% B, flow rate=1.0 mL/min, 20 min), or as as described in the LC-MS Method tables.

Unless indicated otherwise, preparative HPLC was carried out on 250×21.2 mm C-18 column using gradient conditions (10-100% B, flow rate=10.0 mL/min, 20 min). The eluents used were: solvent A (H₂O with 0.1% TFA) and solvent B (CH₃CN with 0.1% TFA). Final products were typically purified via reversed-phase HPLC, PTLC, or flash column chromatography. The abbreviation TFA refers to trifluoroacetic acid.

LC-MS Method 01

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 1 | 0.8 |
| | | 0.40 | 1 | 0.8 |
| | | 3.40 | 90 | 0.8 |
| | | 3.90 | 100 | 0.8 |
| | | 3.91 | 1 | 0.8 |
| | | 4.00 | 1 | 1.0 |
| | | 4.50 | 1 | 1.0 |

| | | |
|---|---|---|
| | Post time(min) | 0 |
| | Column Temp | 50° C. |
| | Detector | DAD |
| MS | Ionization source | ESI |
| | Drying Gas | N₂ |
| | Drying Gas Flow | 10(L/min) |
| | Nebulizer Pressure | 40(psi) |
| | Drying Gas | 350° C. |
| | Temperature | |
| | Capillary Voltage | 2500(V) |
| | MS Polarity | Positive |
| | MS Mode | Scan |
| | Mass Range | 100-1500 |

LC-MS Method 10

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 10 | 0.8 |
| | | 0.40 | 10 | 0.8 |
| | | 3.40 | 100 | 0.8 |
| | | 3.90 | 100 | 0.8 |
| | | 3.91 | 10 | 0.8 |
| | | 4.00 | 10 | 1.0 |
| | | 4.50 | 10 | 1.0 |

| | | |
|---|---|---|
| | Post time(min) | 0 |
| | Column Temp | 50° C. |
| | Detector | DAD |
| MS | Ionization source | ESI |
| | Drying Gas | N₂ |
| | Drying Gas Flow | 10(L/min) |
| | Nebulizer Pressure | 40(psi) |
| | Drying Gas | 350° C. |
| | Temperature | |
| | Capillary Voltage | 2500(V) |
| | MS Polarity | Positive |
| | MS Mode | Scan |
| | Mass Range | 100-1500 |

LC-MS Method 25

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 25 | 0.8 |
| | | 0.40 | 25 | 0.8 |
| | | 3.40 | 100 | 0.8 |
| | | 3.90 | 100 | 0.8 |
| | | 3.91 | 25 | 0.8 |
| | | 4.00 | 25 | 1.0 |
| | | 4.50 | 25 | 1.0 |

| | | |
|---|---|---|
| | Post time(min) | 0 |
| | Column Temp | 50° C. |
| | Detector | DAD |
| MS | Ionization source | ESI |
| | Drying Gas | N₂ |
| | Drying Gas Flow | 10(L/min) |
| | Nebulizer Pressure | 40(psi) |
| | Drying Gas | 350° C. |
| | Temperature | |
| | Capillary Voltage | 2500(V) Positive |
| | MS Polarity | Positive |
| | MS Mode | Scan |
| | Mass Range | 100-1500 |

LC-MS METHOD 40

| Instrument | | Agilent 1100 LC & Agilent G1956A | | |
|---|---|---|---|---|
| Software | | Agilent Chemstation Rev. B. 04.03[16] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 40 | 0.8 |
| | | 0.40 | 40 | 0.8 |
| | | 3.40 | 100 | 0.8 |
| | | 3.90 | 100 | 0.8 |
| | | 3.91 | 40 | 0.8 |
| | | 4.00 | 40 | 1.0 |
| | | 4.50 | 40 | 1.0 |

| | | |
|---|---|---|
| | Post time(min) | 0 |
| | Column Temp | 50° C. |
| | Detector | DAD(Agilent 1100)/ELSD(Agilent 1260 Infinity) |
| MS | Ionization source | ESI |
| | Drying Gas | N₂ |
| | Drying Gas Flow | 10(L/min) |
| | Nebulizer Pressure | 2070(Torr) |
| | Drying Gas | 350° C. |
| | Temperature | |
| | Capillary Voltage | 2500(V) Positive |

### Example 1—Synthesis of Compound I-1

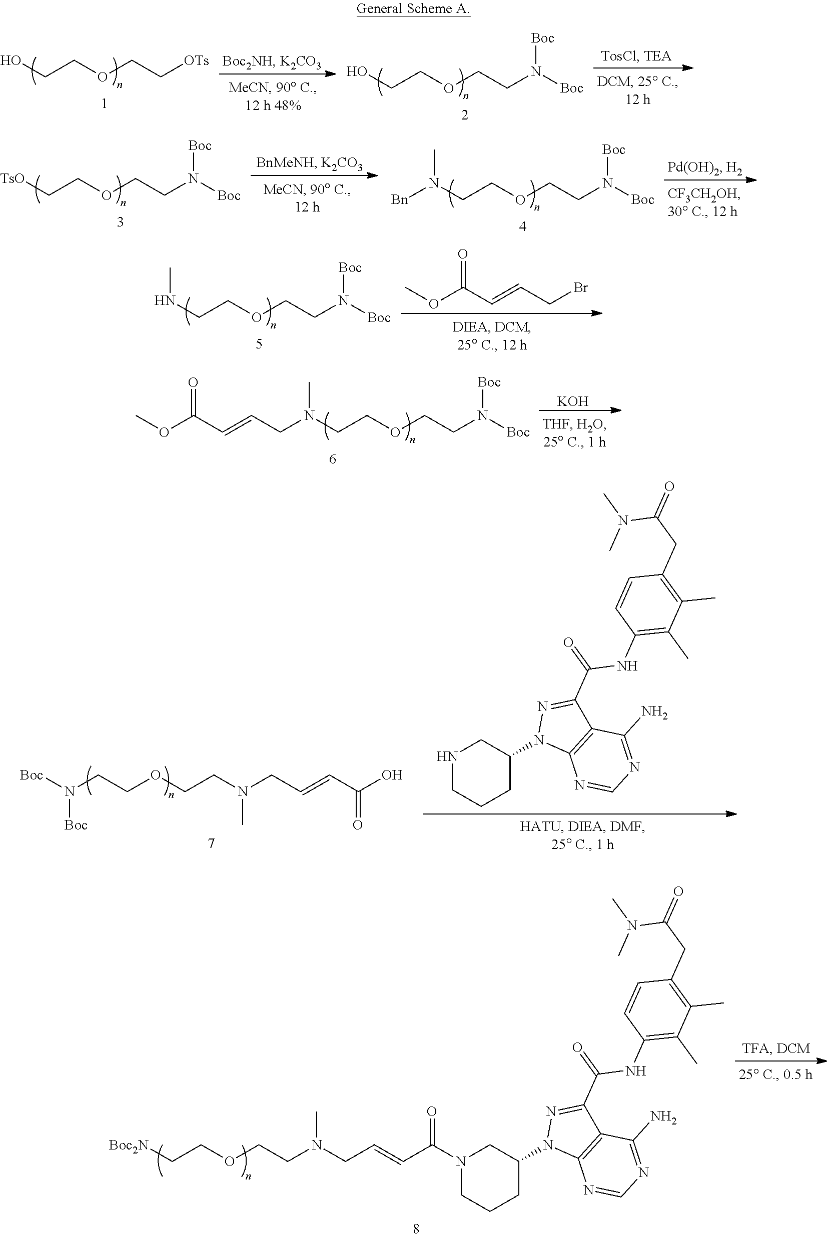

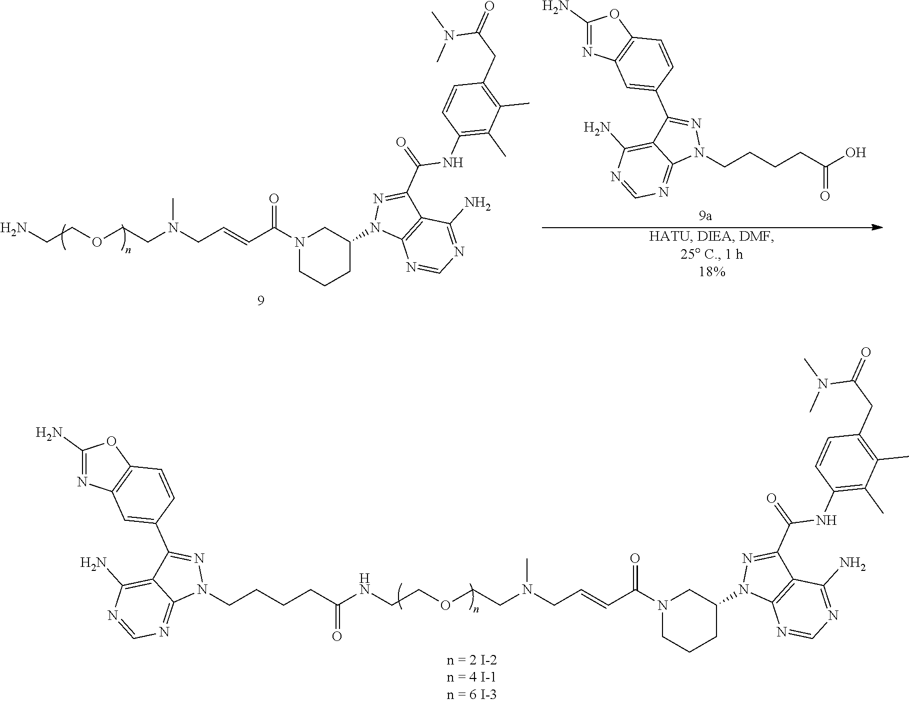

### 1.1 Preparation of Compound 2

To a solution of 2-[2-[2-[2-(2-hydroxyethoxy)ethoxy]ethoxy]ethoxy]ethyl 4-methylbenzenesulfonate (15 g, 38 mmol, 1.0 equiv) and tert-butyl N-tert-butoxy-carbonylcarbamate (8.3 g, 38 mmol, 1.0 equiv) in MeCN (200 mL) was added K₂CO₃ (10 g, 76 mmol, 2.0 equiv). The mixture was stirred at 90° C. for 12 h. The mixture was purified by prep-HPLC (column: Kromasil Eternity XT 250*80 mm*10 um; mobile phase: [water(10 mM NH₄HCO₃)-ACN]; B%: 25%-55%, 20 min) to give the desired product tert-butyl N-tert-butoxycarbonyl-N-[2-[2-[2-[2-(2-hydroxyethoxy)ethoxy]ethoxy]ethoxy]ethyl]carbamate (8.0 g, 18 mmol, 48% yield) as a yellow oil. LC-MS: MS (ES⁺): m/z=238.3 [M+H⁺-200].

### 1.2 Preparation of Compound 3

To a solution of tert-butyl-N-tert-butoxycarbonyl-N-[2-[2-[2-[2-(2-hydroxyethoxy)ethoxy]ethoxy]ethoxy]ethyl]carbamate (5.5 g, 13 mmol, 1.0 equiv) in DCM (20 mL) were added 4-methylbenzenesulfonyl chloride (3.6 g, 19 mmol, 1.5 equiv) and TEA (3.8 g, 38 mmol, 3.0 equiv). The mixture was stirred at 25° C. for 12 h. The mixture was purified by silica gel column chromatography (Dichloromethane: Methanol=100/1 to 30/1) to give desired product 2-1-[2-[2-1[2-[2-1[bis(tert-butoxycarbonyl)amino]ethoxy]ethoxy]ethoxy]ethoxy]ethyl-4-methylbenzenesulfonate (6.0 g, 10 mmol) as a yellow oil. LC-MS: MS (ES+): m/z=392.0 [M+H⁺-200].

### 1.3 Preparation of Compound 4

To a solution of 2-[2-[2-[2-[2-[bis(tert-butoxycarbonyl)amino]ethoxy]ethoxy]ethoxy]ethoxy]ethyl-4-methylbenzenesulfonate (9.0 g, 15 mmol, 1.0 equiv) in MeCN (100 mL) were added N-methyl-1-phenyl-methanamine (1.8 g, 15 mmol, 1.0 equiv), K₂CO₃ (2.3 g, 17 mmol, 1.1 equiv). The mixture was stirred at 90° C. for 12 h. The mixture was purified by silica column chromatography on silica gel (Dichloromethane: Methanol=100/1 to 30/1) to give desired product tert-butyl-N-[2-1[2-[2-1[2-1[2-[benzyl(methyl)amino]ethoxy]ethoxy]ethoxy]ethoxy]ethyl]-N-tert-butoxycarbonyl-carbamate (8.0 g, 15 mmol) as a yellow oil. LC-MS: MS (ES+): m/z=541.5 [M+H⁺].

### 1.4 Preparation of Compound 5

To a solution of tert-butyl-N-[2-[2-1[2-[2-1[2-[benzyl(methyl)amino]ethoxy]ethoxy]ethoxy]ethoxy]ethyl]-N-tert-butoxycarbonyl-carbamate (8.0 g, 15 mmol, 1.0 equiv) in CF₃CH₂OH (80 mL) was added Pd(OH)₂ (0.8 g, 0.6 mmol, 10% purity). The mixture solution was purged with H₂ for 3 times. The mixture was stirred at 30° C. for 12 h under H₂ atmosphere (50 psi). The reaction mixture was filtered. The filtrate was concentrated under reduced pressure to give a desired product tert-butyl-N-tert-butoxycarbonyl-N-[2-[2-[2-[2-[2-(methylamino)ethoxy]ethoxy]ethoxy]ethoxy]ethyl]carbamate (6.0 g, 13 mmol) as a yellow oil. LC-MS: MS (ES+): m/z=451.5 [M+H⁺].

### 1.5 Preparation of Compound 6

To a solution of tert-butyl-N-tert-butoxycarbonyl-N-[2-[2-[2-[2-[2-(methylamino) ethoxy]ethoxy]ethoxy]ethoxy]ethyl]carbamate (6.0 g, 13 mmol, 1.0 equiv) in DCM (60 mL) were added methyl (E)-4-bromobut-2-enoate (2.2 g, 12 mmol, 0.9 equiv) and DIEA (2.6 g, 20 mmol, 3.5 mL, 1.5 equiv). The mixture was stirred at 25° C. for 12 h. The reaction mixture was purified by prep-HPLC (column: Phenomenex luna C18 (250*70 mm, 10 um); mobile phase: [water(1 OmM NH₄HCO₃)-ACN]; B%: 50%-80%, 18 min) to give a desired product methyl-(E)-4-[2-[2-[2-[2-[2-[bis(tert-butoxycarbonyl)amino]ethoxy]ethoxy]ethoxy]ethoxy]ethyl-methyl-amino]but-2-enoate (4.4 g, 8.0 mmol, 60% yield) as a yellow oil. ¹H NMR (400 MHz, CHLOROFORM-d): δ 6.98 (td, 1H, J=6.2, 15.8 Hz), 6.01 (d, 1H, J=15.8 Hz), 3.81-3.76 (m, 2H), 3.75-3.73 (m, 3H), 3.65-3.59 (m, 16H), 3.25 (d, 2H, J=5.6 Hz), 2.65 (t, 2H, J=5.6 Hz), 2.32 (s, 3H), 1.50 (s, 18H). LC-MS: MS (ES⁺): m/z=549.3 [M+H⁺].

### 1.6 Preparation of Compound 7

To a solution of methyl-(E)-4-[2-[2-[2-[2-[2-[bis(tert-butoxycarbonyl)amino]ethoxy]ethoxy]ethoxy]ethoxy]ethyl-methyl-amino]but-2-enoate (300 mg, 547 μmol, 1.0 equiv) in H₂O (2 mL) and THF (2 mL) was added KOH (46 mg, 0.82 mmol, 1.5 equiv). The mixture was stirred at 25° C. for 1 h. The reaction mixture was concentrated under reduced pressure to give crude product (E)-4-[2-[2-[2-[2-[2-[bis(tert-butoxycarbonyl)amino]ethoxy]ethoxy]ethoxy]ethoxy]ethyl-methyl-amino]but-2-enoic acid (290 mg, 542 μmol) as a yellow oil. LC-MS: MS (ES+): m/z=535.5 [M+H⁺].

### 1.7 Preparation of Compound 8

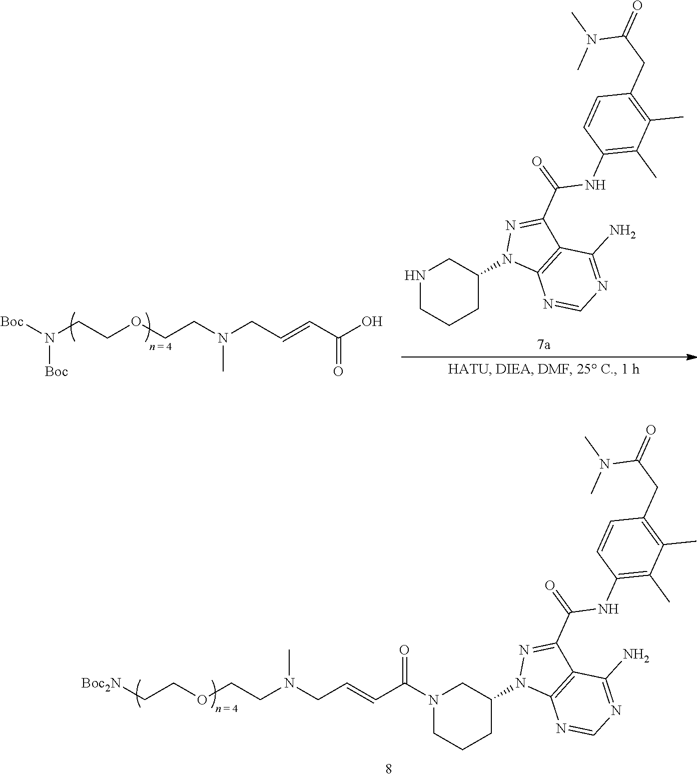

To a solution of 4-amino-N-[4-[2-(dimethylamino)-2-oxo-ethyl]-2,3-dimethyl-phenyl]-1-[(3R)-3-piperidyl]pyrazolo[3,4-d]pyrimidine-3-carboxamide (255 mg, 452 μmol, 1.0 equiv, TFA salt) in DMF (4 mL) were added HATU (240 mg, 633 μmol, 1.4 equiv), (E)-4-[2-[2-[2-[2-[2-[bis(tert-butoxycarbonyl)amino]ethoxy]ethoxy]ethoxy]ethoxy]ethyl-methyl-amino]but-2-enoic acid (290 mg, 542 μmol, 1.2 equiv) and DIEA (175 mg, 1.36 mmol, 3.0 equiv). The mixture was stirred at 25° C. for 1 h. The mixture was purified by prep-HPLC (column: Waters Xbridge C18 150*50 mm* 10 um; mobile phase: [water(10 mM NH₄HCO₃)-ACN]; B%: 32%-62%, 10 min) to give tert-butyl-N-[2-[2-[2-[2-[2-[[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-methyl-amino]ethoxy]ethoxy]ethoxy]ethoxy]ethyl]-N-tert-butoxycarbonyl-carbamate (410 mg, 414 μmol) as a yellow oil. LC-MS: MS (ES⁺): m/z=867.8 [M+H⁺-100].

### 1.8 Preparation of Compound 9

To a solution of tert-butyl-N-[2-[2-[2-[2-[2-[[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-methyl-amino]ethoxy]ethoxy]ethoxy]ethoxy]ethyl]-N-tert-butoxycarbonyl-carbamate (135 mg, 139 μmol, 1.0 equiv) in DCM (1 mL) was added TFA (462 mg, 4.10 mmol, 29.0 equiv). The mixture was stirred at 25° C. for 0.5 h. The reaction mixture was concentrated under reduced pressure to give desired product 4-amino-1-[(3R)-1-[(E)-4-[2-[2-[2-[2-(2-aminoethoxy)ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (120 mg, 136 μmol, TFA salt) as a yellow oil.

### 1.9 Preparation of Compound I-1

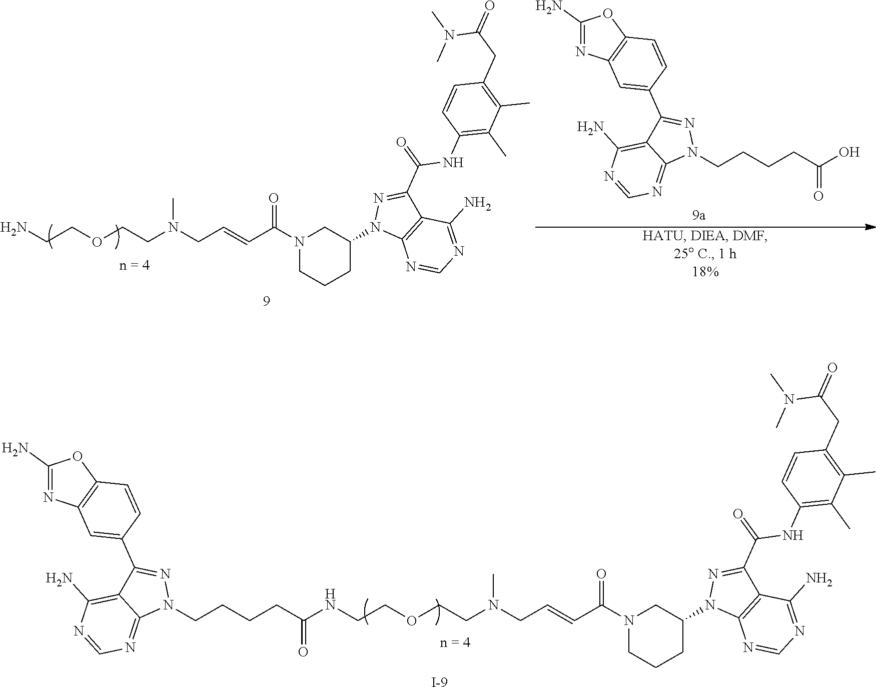

To a solution of 4-amino-1-[(3R)-1-[(E)-4-[2-[2-[2-[2-(2-aminoethoxy)ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (120 mg, 136 μmol, 1.0 equiv, TFA salt) in DMF (2 mL) were added HATU (67 mg, 177 μmol, 1.3 equiv), 5-[4-amino-3-(2-amino-1,3-benzoxazol-5-yl)pyrazolo[3,4-d]pyrimidin-1-yl]pentanoic acid (50 mg, 136 mol, 1.0 equiv) and DIEA (53 mg, 408 μmol, 71 μL, 3.0 equiv). The mixture was stirred at 25° C. for 1 h. The mixture was purified by prep-HPLC (column: Phenomenex luna C18 150*25 mm* 10 um; mobile phase: [water(0.225% FA)-ACN]; B%: 7%-37%, 10 min) to give 4-amino- [(3R)-1-[(E)-4-[2-[2-[2-[2-[2-[5-[4-amino-3-(2-amino-1,3-benzoxazol-5-yl)pyrazolo[3,4-d]pyrimidin-1-yl]pentanoylamino]ethoxy]ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (27 mg, 24 μmol, 18% yield, 100% purity) as a white solid. ¹H NMR (400 MHz, METHANOL-d₄): δ8.26-8.19 (m, 2H), 7.50 (s, 1H), 7.44-7.39 (m, 1H), 7.38-7.30 (m, 2H), 6.99 (d, 1H, J=8.0 Hz), 6.82-6.54 (m, 2H), 4.62-4.52 (m, 1H), 4.39 (t, 2H, J=6.4 Hz), 4.26-4.16 (m, 1H), 4.14-3.86 (m, 2H), 3.78 (s, 2H), 3.68-3.39 (m, 20H), 3.11 (s, 3H), 2.99 (s, 3H), 2.83-2.65 (m, 2H), 2.44 (s, 1H), 2.35 (s, 3H), 2.30-2.22 (m, 6H), 2.20 (s, 4H), 2.15-1.97 (m, 1H), 1.94 (t, 2H, J=7.0 Hz), 1.79-1.67 (m, 1H), 1.65-1.55 (m, 2H). LC-MS: MS (ES⁺): RT=1.948 min, m/z=1116.4 [M+H⁺], LC-MS METHOD 10.

### Example 2—Synthesis of Compound I-2 (Synthesized According to General Scheme a in a Similar Manner to Compound I-1)

4-amino-1-[(3R)-1-[(E)-4-[2-[2-[2-[5-[4-amino-3-(2-amino-1,3-benzoxazol-5-yl)pyrazolo[3,4-d]pyrimidin-1-yl]pentanoylamino]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (62 mg, 58 μmol, 43% yield, 95% purity) as a white solid. ¹H NMR (400 MHz, MeOD): δ8.29-8.15 (m, 2H), 7.52-7.27 (m, 4H), 7.03-6.94 (m, 1H), 6.84-6.38 (m, 2H), 4.46-4.30 (m, 2H), 4.24-3.88 (m, 2H), 3.80-3.76 (m, 2H), 3.76-3.62 (m, 2H), 3.55 (s, 3H), 3.49-3.41 (m, 5H), 3.25-3.16 (m, 2H), 3.14-3.08 (m, 4H), 3.04-2.89 (m, 4H), 2.65-2.55 (m, 1H), 2.53-2.45 (m, 1H), 2.30-2.23 (m, 6H), 1.98-1.85 (m, 2H), 1.66-1.56 (m, 2H), 1.38-1.34 (m, 10H). LC-MS: MS (ES+): RT=1.924 min, m/z=1028.4 [M+H⁺], LC-MS METHOD 10.

### Example 3—Synthesis of Compound I-3 (Synthesized According to General Scheme a in a Similar Manner to Compound I-1)

4-amino-1-[(3R)-1-[(E)-4-[2-[2-[2-[2-[2-[2-[2-[5-[4-amino-3-(2-amino-1,3-benzoxazol-5-yl) pyrazolo[3,4-d]pyrimidin-1-yl]pentanoylamino]ethoxy]ethoxy]ethoxy]ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (86 mg, 72 μmol, 53% yield, 100% purity) as an off-white solid. ¹H NMR (400 MHz, CD₃OD): δ8.29-8.20 (m, 2H), 7.51 (d, J=1.2 Hz, 1H), 7.43 (d, J=8.0 Hz, 1H), 7.39-7.31 (m, 2H), 7.00 (d, J=8.0 Hz, 1H), 6.82-6.62 (m, 2H), 6.58-6.47 (m, 1H), 4.41 (t, J=6.8 Hz, 2H), 4.13-3.89 (m, 2H), 3.79 (s, 2H), 3.63-3.58 (m, 8H), 3.55 (d, J=8.0 Hz, 14H), 3.51-3.46 (m, 6H), 3.17 (s, 1H), 3.11 (s, 3H), 3.03-2.98 (m, 4H), 2.66 (s, 1H), 2.54 (d, J=3.5 Hz, 1H), 2.44-2.30 (m, 3H), 2.29-2.15 (m, 13H), 2.01-1.91 (m, 2H), 1.79-1.60 (m, 3H). LC-MS: MS (ES+): RT=2.060 min, m/z=1205.5 [M+H⁺], LC-MS METHOD 10.

### Example 4—Synthesis of Compound I-4

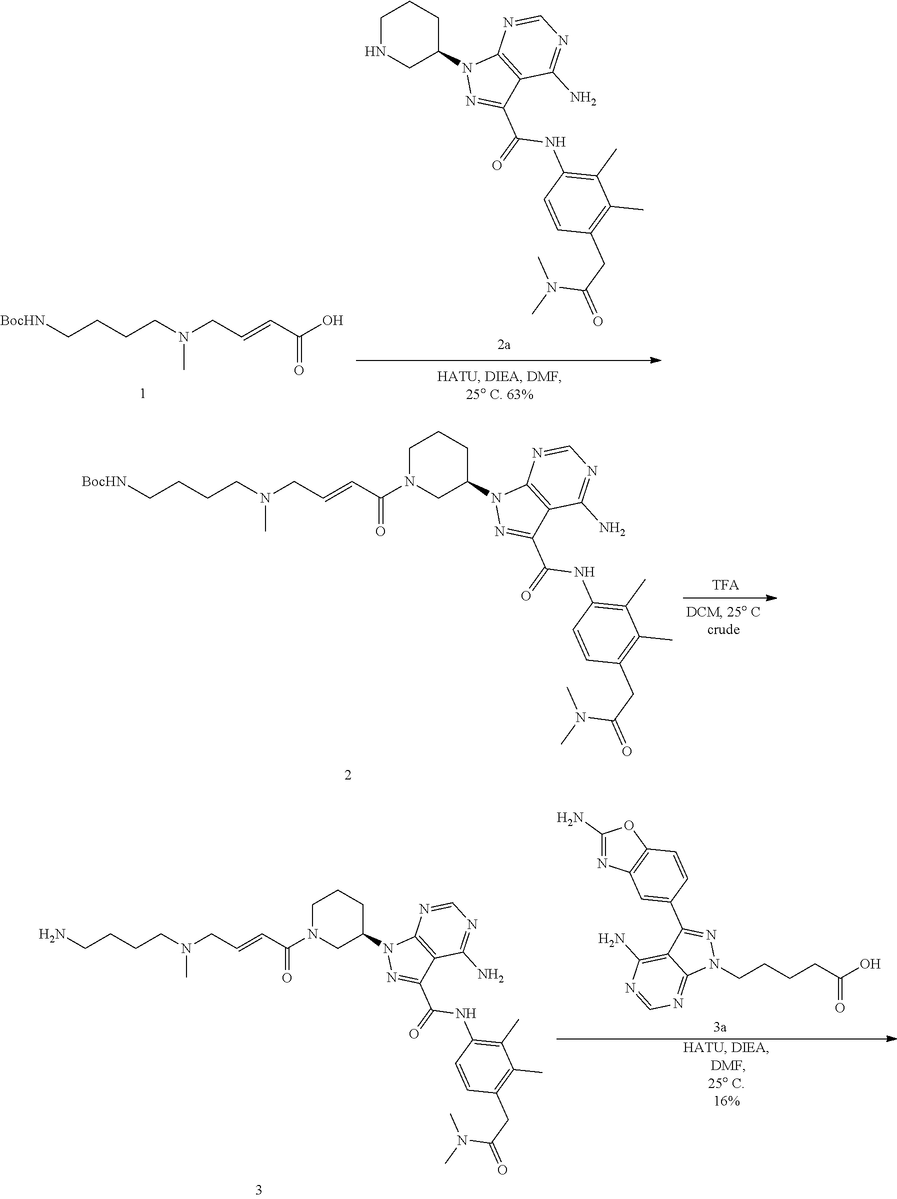 [SMILES: CN(C/C=C/C(=O)O)CCCCNC(=O)OC(C)(C)C ; Cc1c(CC(=O)N(C)C)ccc(NC(=O)c2nn([C@@H]3CCCNC3)c3ncnc(N)c23)c1C ; Cc1c(CC(=O)N(C)C)ccc(NC(=O)c2nn([C@@H]3CCCN(C(=O)/C=C/CN(C)CCCCNC(=O)OC(C)(C)C)C3)c3ncnc(N)c23)c1C ; Cc1c(CC(=O)N(C)C)ccc(NC(=O)c2nn([C@@H]3CCCN(C(=O)/C=C/CN(C)CCCCN)C3)c3ncnc(N)c23)c1C ; Nc1nc2cc(-c3nn(CCCCC(=O)O)c4ncnc(N)c34)ccc2o1]

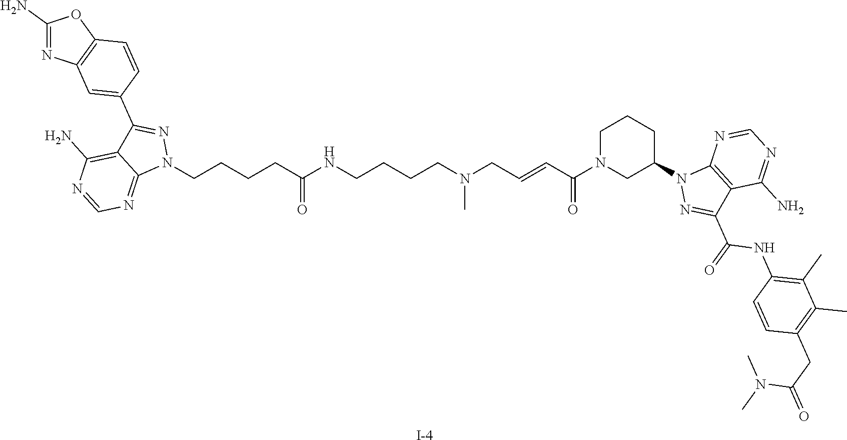

### 4.1 Preparation of Compound 2

To a solution of (E)-4-[4-(tert-butoxycarbonylamino)butyl-methyl-amino]but-2-enoic acid (180 mg, 628 μmol, 1 equiv), 4-amino-N-[4-[2-(dimethylamino)-2-oxo-ethyl]-2,3-dimethyl-phenyl]-1-[(3R)-3-piperidyl]pyrazolo[3,4-d]pyrimidine-3-carboxamide (354 mg, 628 mol, 1 equiv, TFA salt) and DIEA (243 mg, 1.89 mmol, 3 equiv) in DMF (3 mL) was added HATU (286 mg, 754 μmol, 1.2 equiv). The mixture solution was stirred at 25° C. for 1 h. The solution was purified by prep-HPLC (column: Waters Xbridge 150*25 mm* 5 um; mobile phase: [water(1 OmM NH₄HCO₃)-ACN]; B%: 35%-56%, 6 min) to give tert-butyl N-[4-[[(E)-4-[(3R)-3-[4-amino-3-[1[4-[2-2(dimethylamino)-2-oxo-ethyl]-2,3-dimethyl-phenyl]carbamoyl]pyrazolo [3,4-d]pyrimidin-1-yl]-1-piperidyl]-4-oxo-but-2-enyl]-methyl-amino]butyl]carbamate (285 mg, 396 μmol, 63% yield) as a yellow solid. ¹H NMR (400 MHz, CDCl₃) δ8.87 (m, 2H), 8.35 (s, 1H), 7.64-7.47 (m, 1H), 7.05 (d, J=8.4 Hz, 1H), 6.90-6.73 (m, 1H), 6.72-6.25 (m, 1H), 5.98-5.79 (m, 1H), 5.08-4.61 (m, 2H), 4.28-3.81 (m, 1H), 3.75-3.57 (s, 2H), 3.48-3.09 (m, 3H), 3.06 (s, 3H), 3.02 (s, 3H), 2.57-2.34 (m, 2H), 2.33-2.18 (m, 1OH), 2.18-1.93 (m, 2H), 1.89-1.50 (m, 8H), 1.49-1.26 (m, 13H). LC-MS: MS (ES+): m/z=719.5 [M+H⁺].

### 4.2 Preparation of Compound 3.

To a solution of tert-butyl N-[4-[[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-methyl-amino]butyl]carbamate (285 mg, 396 μmol, 1 equiv) in DCM (4 mL) was added TFA (2 mL, 68 equiv). The mixture solution was stirred at 25° C. for 0.5 h. Then the solution was concentrated to give 4-amino-1-[(3R)-1-[(E)-4-[4-aminobutyl(methyl)amino]but-2-enoyl]-3-piperidyl]-N-[4-1[2-(dimethylamino)-2-oxo-ethyl]-2,3-dimethyl-phenyl]pyrazolo[3,4-d]pyrimidine-3-carboxamide (360 mg, crude, 2 TFA) as a yellow oil. LC-MS: MS (ES+): m/z=619.3 [M+H⁺].

### 4.3 Preparation of Compound I-4

To a solution of 4-amino-1-[(3R)-1-[(E)-4-[4-aminobutyl(methyl)amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (115 mg, 136 μmol, 1 equiv, 2TFA salt) and 5-[4-amino-3-(2-amino-1,3-benzoxazol-5-yl)pyrazolo[3,4-d]pyrimidin-1-yl]pentanoic acid (50 mg, 136 μmol, 1 equiv) in DMF (3 mL) was added DIEA (53 mg, 0.41 mmol, 3 equiv) and HATU (62 mg, 0.16 mmol, 1.2 equiv). The mixture solution was stirred at 25° C. for 1 h. The solution was purified by prep-HPLC (column: Phenomenex luna C18 150*25 mm* 10 um; mobile phase: [water(0.225% FA)-ACN]; B%: 8%-38%, 10 min) to give 4-amino-[(3R)-1-[(E)-4-[4-[5-[4-amino-3-(2-amino-1,3-benzoxazol-5-yl)pyrazolo[3,4-d]pyrimidin-1-yl]pentanoylamino]butyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (23 mg, 22 μmol, 16% yield, 100% purity, FA salt) as a yellow solid. ¹H NMR (400 MHz, MeOH-a₄) δ 8.48 (s, 1H), 8.29-8.17 (m, 2H), 7.50 (s, 1H), 7.43 (d, J=8.0 Hz, 1H), 7.38-7.28 (m, 2H), 6.99 (d, J=8.8 Hz, 1H), 6.86-6.53 (m, 2H), 4.66-4.48 (m, 1H), 4.45-4.35 (m, 2H), 4.28-4.16 (m, 1H), 4.20-4.15 (m, 1H), 4.17-3.89 (m, 1H), 3.79 (s, 2H), 3.51-3.36 (m, 2H), 3.20-3.06 (m, 5H), 2.99 (s, 3H), 2.74-2.54 (m, 2H), 2.47-2.32 (m, 4H), 2.32-2.11 (m, 9H), 2.00-1.85 (m, 2H), 1.74-1.37 (m, 7H). LC-MS: MS (ES⁺): RT=2.254 min, m/z=968.5 [M+H⁺], LC-MS METHOD 01.

### Example 5—Synthesis of Compound I-5

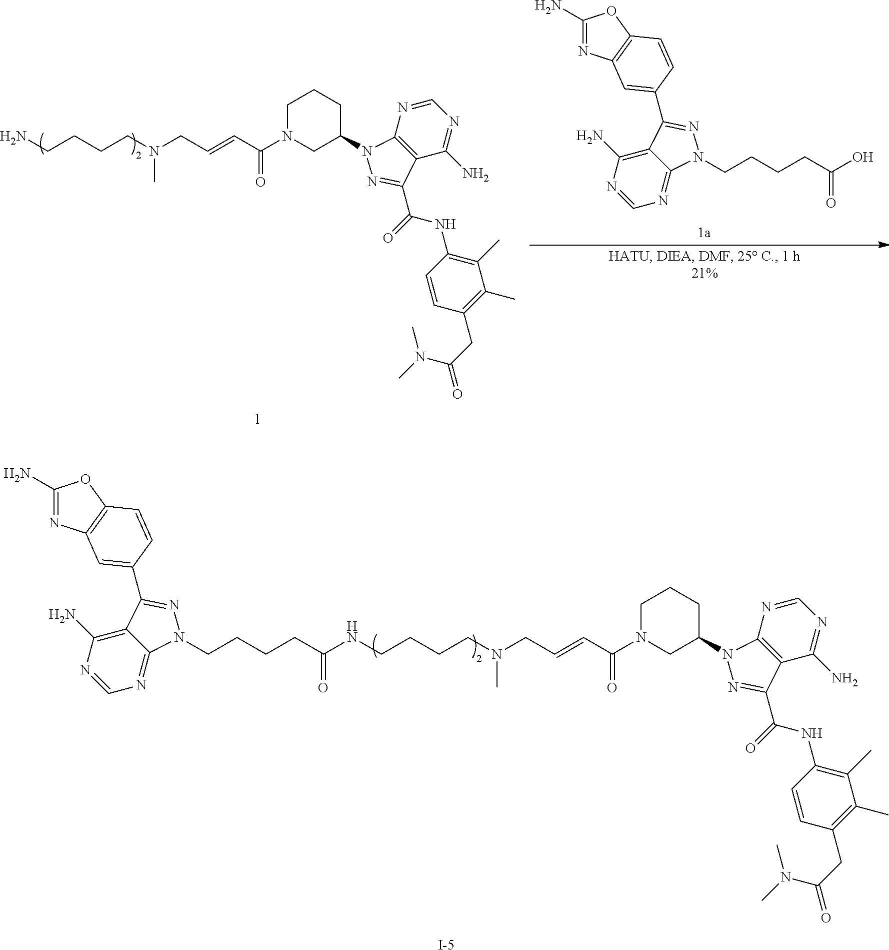

To a solution of 5-[4-amino-3-(2-amino-1,3-benzoxazol-5-yl)pyrazolo[3,4-d]pyrimidin-1-yl]pentanoic acid (52 mg, 0.14 mmol, 1.0 equiv) in DMF (2 mL) were added HATU (70 mg, 0.18 mmol, 1.3 equiv), 4-amino-1-[(3R)-1-[(E)-4-[8-aminooctyl(methyl) amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (128 mg, 142 μmol, 1.0 equiv, 2 TFA salt) and DIEA (55 mg, 0.42 mmol, 3.0 equiv). The mixture was stirred at 25° C. for 1 h. The mixture was purified by prep-HPLC (column: Phenomenex luna C18 150*25 mm* 10 um; mobile phase: [water(0.225% FA)-ACN]; B%: 8%-44%, 12 min) to give 4-amino-1-[(3R)-1-[(E)-4-[8-[5-[4-amino-3-(2-amino-1,3-benzoxazol-5-yl)pyrazolo[3,4-d]pyrimidin-1-yl]pentanoylamino]octyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (30 mg, 29 μmol, 21% yield, 100% purity) as a white solid. ¹H NMR (400 MHz, METHANOL-d₄): δ8.52 (s, 1H), 8.26-8.17 (m, 2H), 7.49 (s, 1H), 7.42-7.28 (m, 3H), 6.97 (d, 1H, J=8.4 Hz), 6.85-6.55 (m, 2H), 4.39 (t, 2H, J=6.6 Hz), 4.24-3.88 (m, 2H), 3.76 (s, 2H), 3.71-3.32 (m, 4H), 3.16-3.03 (m, 5H), 2.98 (s, 3H), 2.72-2.50 (m, 2H), 2.50-2.33 (m, 4H), 2.30-2.12 (m, 10H), 2.11-1.81 (m, 3H), 1.76-1.51 (m, 4H), 1.50-1.36 (m, 3H), 1.30-1.13 (m, 8H). LC-MS: MS (ES′): RT=2.395 min, m/z=1024.3 [M+H⁺], LC-MS METHOD 01.

### Example 6—Synthesis of Compound I-6

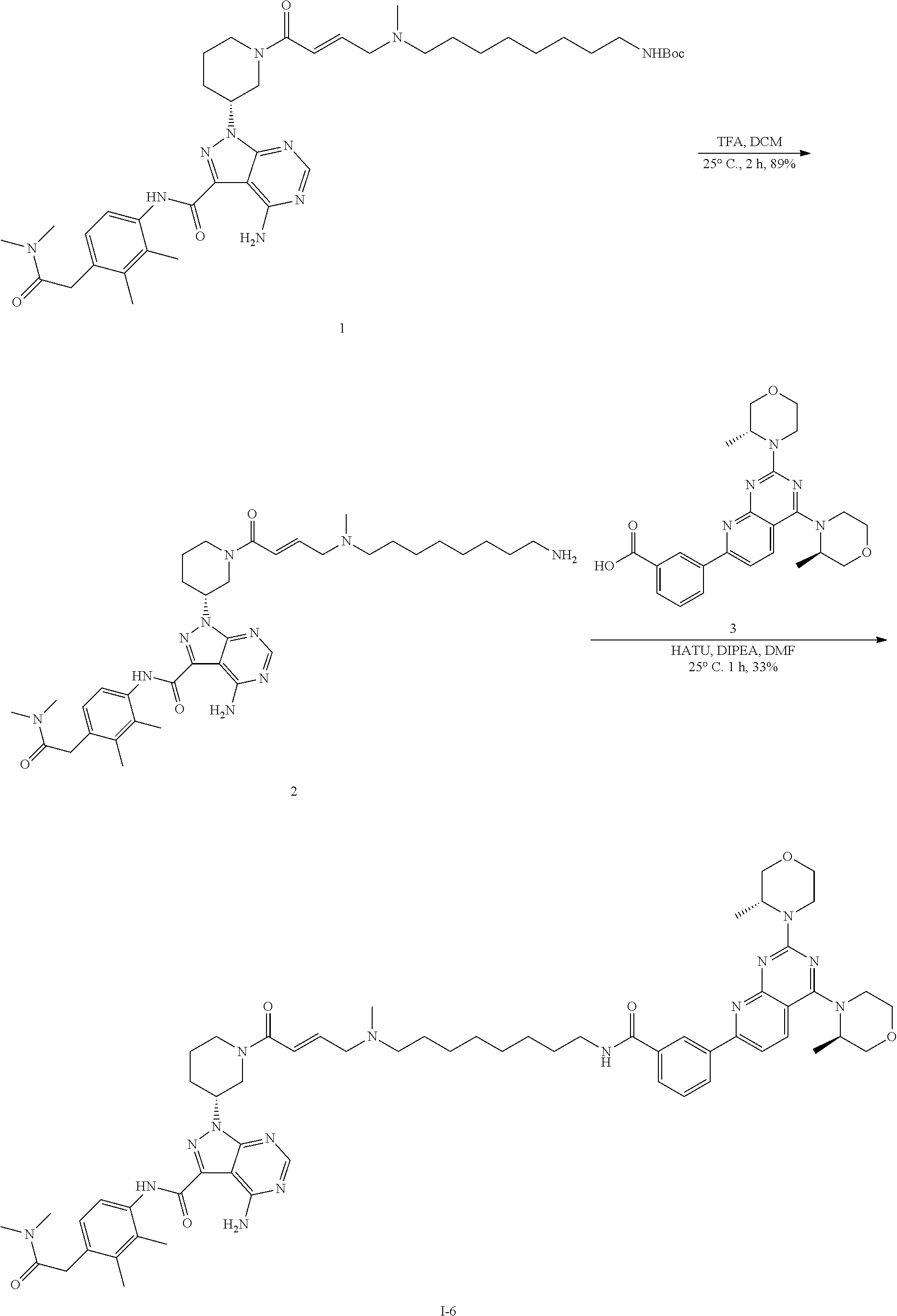

### 6.1 Preparation of Compound 2

A mixture of tert-butyl N-[8-[[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-methyl-amino]octyl]carbamate (230 mg, 296 μmol, 1.0 equiv) in TFA (1 mL) and DCM (3 mL) was degassed and purged with N₂ for 3 times and then the mixture was stirred at 25° C. for 2 h. The mixture was concentrated to give 4-amino-1-[(3R)-1-[(E)-4-[8-aminooctyl (methyl)amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (210 mg, 89% yield, TFA salt) as a colorless oil.

### 6.2 Preparation of Compound I-9

A mixture of 3-[2,4-bis[(3R)-3-methylmorpholin-4-yl]pyrido[2,3-d]pyrimidin-7-yl]benzoic acid (50 mg, 111 μmol, 1.0 equiv), 4-amino-1-[(3R)-1-[(E)-4-[8-aminooctyl (methyl)amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (87 mg, 111 μmol, 1.0 equiv, TFA), DIPEA (71 mg, 556 μmol, 5.0 equiv) and HATU (46 mg, 122 μmol, 1.1 equiv) in DMF (1 mL) was degassed and purged with N₂ for 3 times and then the mixture was stirred at 25° C. for 1 h. The mixture was concentrated and purified by the prep-HPLC (Waters Xbridge 150*25 mm* 5 um; mobile phase: [water(10 mM NH4HCO3)-ACN]; B%: 40%-73%, 9 min) to give 4-amino-1-[(3R)-1-[(E)-4-1[8-1[[3-[2,4-bis [(3R)-3-methylmorpholin-4-yl]pyrido [2,3-d]pyrimidin-7-yl]benzoyl]amino]octyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (43 mg, 33% yield). ¹H NMR (400 MHz, DMSO-d₆): δ8.62 (r s, 3H), 8.36-8.19 (m, 3H), 8.10 (s, 1H), 7.98-7.91 (m, 1H), 7.76-7.67 (m, 1H), 7.65-7.53 (m, 1H), 7.20-7.08 (m, 1H), 7.01-6.90 (m, 1H), 6.73-6.40 (m, 2H), 4.92-4.65 (m, 2H), 4.60-4.42 (m, 2H), 4.05 (s, 1H), 3.96-3.86 (m, 3H), 3.84-3.57 (m, 9H), 3.49-3.39 (m, 2H), 3.31-3.15 (m, 4H), 3.04 (s, 4H), 2.95 (s, 1H), 2.85 (s, 3H), 2.30-1.88 (m, 14H), 1.53 (s, 3H), 1.42-1.16 (m, 16H). LC-MS: MS (ES′): RT=2.568 min, m/z=1106.4 [M+H⁺].

### Example 7—Synthesis of Compound I-7 (Synthesized According to General Scheme B)

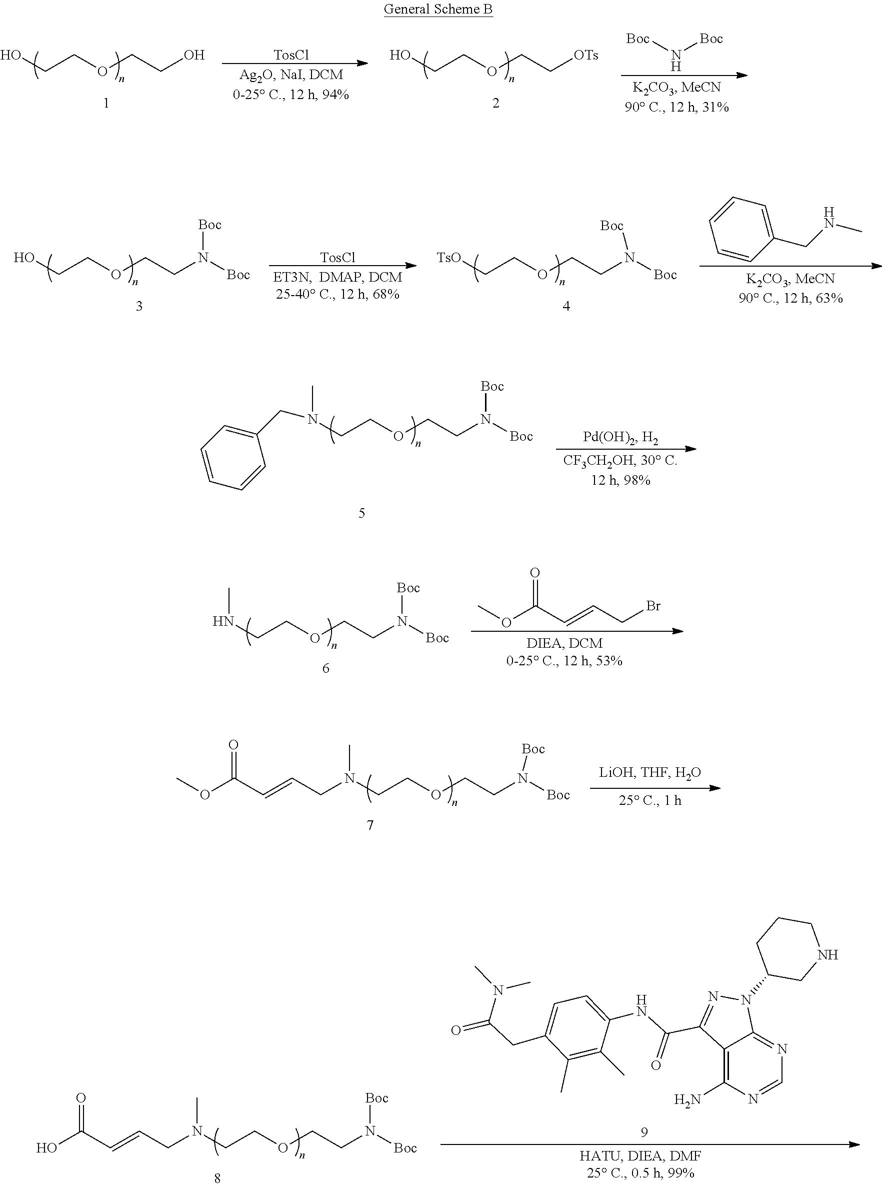

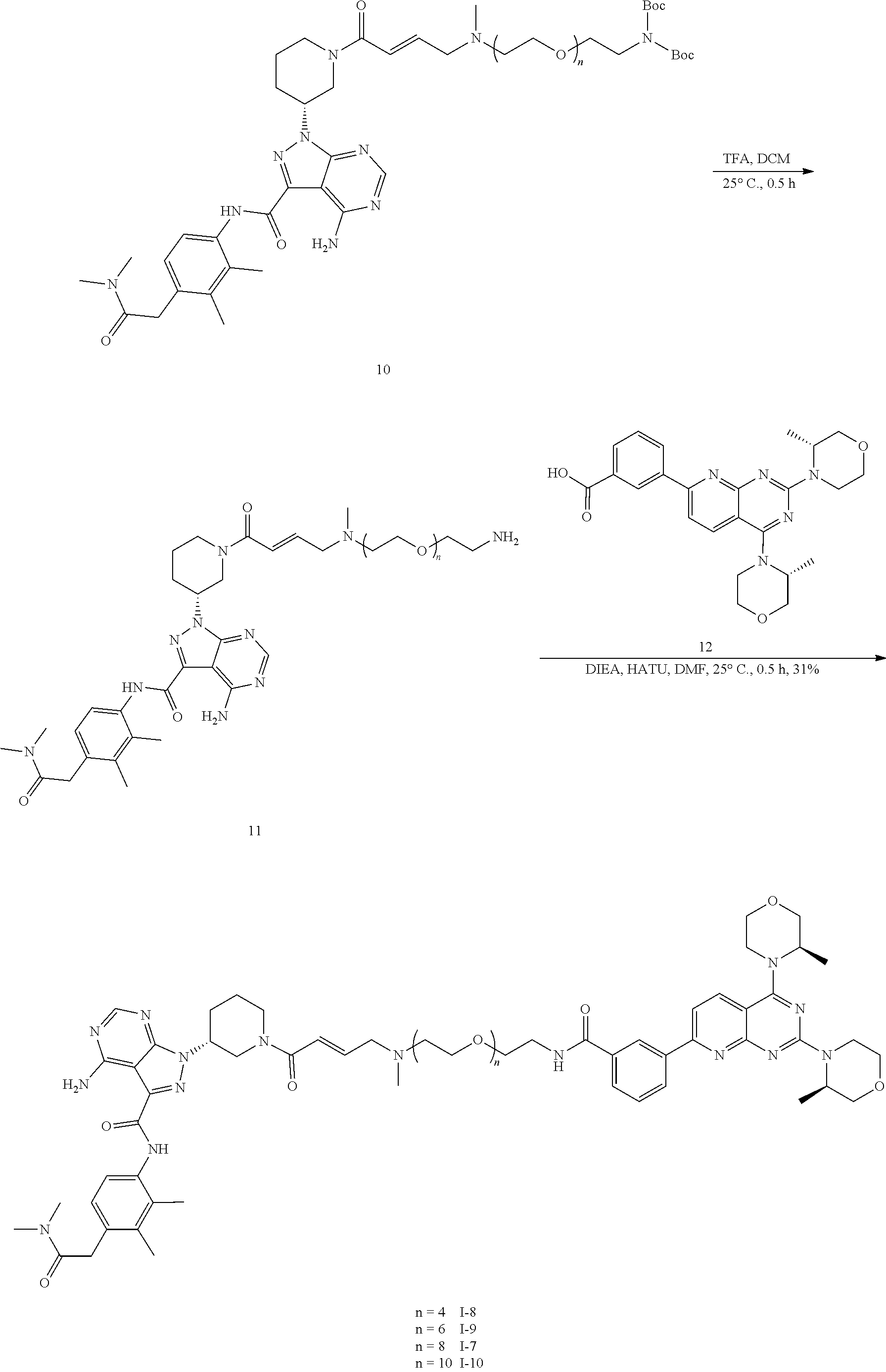

### 7.1 Preparation of Compound 2

To a solution of 2-[[2[2-[2-[2-[2-[2-(2-hydroxyethoxy)ethoxy]ethoxy]ethoxy]ethoxy]ethoxy]ethoxy]ethoxy]ethanol (7 g, 17 mmol, 1 equiv) in DCM (70 mL) was added Ag₂O (5.9 g, 25 mmol, 1.5 equiv), NaI (2.8 g, 18 mmol, 1.1 equiv) and 4-methylbenzenesulfonyl chloride (3.4 g, 18 mmol, 1 equiv) at 0° C. The reaction mixture was stirred at 25° C. for 12 h and concentrated. The residue was diluted with H₂O (100 mL) and extracted with EtOAc (3×100 mL). The combined organic layer was dried, filtered and concentrated. The residue was purified by column chromatography (SiO₂, Petroleum ether/Ethyl acetate=1/1 to 0/1) to give 2-[2-[2-[2-[2-[2-[2-[2-(2-hydroxyethoxy) ethoxy]ethoxy]ethoxy]ethoxy]ethoxy]ethoxy]ethoxy]ethyl 4-methylbenzenesulfonate (9 g, 94% yield) as a colorless oil. ¹H NMR (400 MHz, CDCl₃): δ7.8 (d, 2H, J=8 Hz), 7.34 (d, 2H, J=8 Hz), 3.72-3.58 (m, 36H), 2.45 (s, 3H).

### 7.2 Preparation of Compound 3

To a solution of [2[2-[2-121-[21[2-[2-(2-hydroxyethoxy)ethoxy]ethoxy]ethoxy]ethoxy]ethoxy]ethoxy]ethoxy]ethyl 4-methylbenzenesulfonate (9 g, 16 mmol, 1 equiv), tert-butyl N-tert-butoxycarbonylcarbamate (3.4 g, 16 mmol, 1 equiv) in MeCN (100 mL) was added K₂CO₃ (4.4 g, 32 mmol, 2 equiv). The mixture was stirred at 90° C. for 12 h and concentrated. The residue was purified by prep-HPLC (column: Waters Xbridge 150*25 mm*5 um; mobile phase: [water (10 mM NH4HCO3)-ACN]; B%: 30%-60%, 10 min) to give tert-butyl N-tert-butoxycarbonyl-N-[2-[2-[2-[2-[2-[2-[2-[2-(2-hydroxyethoxy) ethoxy]ethoxy]ethoxy]ethoxy]ethoxy]ethoxy]ethoxy]ethyl]carbamate (3 g, 31% yield) as a colorless oil. ¹H NMR (400 MHz, CDCl3): δ3.79-3.62 (m, 34H), 2.06 (s, 3H), 1.51-1.45 (m, 18H). LC-MS: MS (ES⁺): m/z=631.3[M+18H⁺].

### 7.3 Preparation of Compound 4

To a solution of tert-butyl N-tert-butoxycarbonyl-N-[2-2-[2-2-[2-[2-[2-[2-[2-(2-hydroxyethoxy) ethoxy]ethoxy]ethoxy]ethoxy]ethoxy]ethoxy]ethoxy]ethyl]carbamate (2 g, 3 mmol, 1 equiv), Et₃N (989 mg, 9.8 mmol, 3 equiv) and DMAP (20 mg, 164 μmol, 0.1 equiv) in DCM (20 mL) was added TosCl (1.2 g, 6.5 mmol, 2 equiv) at 25° C. The mixture was stirred at 40° C. for 12 h and concentrated. The residue was purified by prep-HPLC (column: Waters Xbridge C18 150*50 mm*l0 um; mobile phase: [water (10 mM NH4HCO3)-ACN]; B%: 4%-34%, min) to give 2-[[2[2-[2-[2-[2-[2-[2-[bis(tert-butoxycarbonyl)amino]ethoxy]ethoxy]ethoxy]ethoxy]ethoxy]ethoxy]ethoxy]ethoxy]ethyl 4-methylbenzenesulfonate (1.7 g, 68% yield) as a colorless oil. LC-MS: MS (ES′): RT=0.95 min, m/z=785.2 [M+18H⁺].

### 7.4 Preparation of Compound 5

To a solution of [2-[2-[2-[2-[2-[2-[2-[2-[bis(tert-butoxycarbonyl)amino]ethoxy]ethoxy]ethoxy]ethoxy]ethoxy]ethoxy]ethoxy]ethoxy]ethyl 4-methylbenzenesulfonate (1.3 g, 1.7 mmol, 1 equiv), N-methyl-1-phenyl-methanamine (410 mg, 3.4 mmol, 437 μL, 2 equiv) in MeCN (15 mL) was added K₂CO₃ (702 mg, 5 mmol, 3 equiv). The mixture was stirred at 90° C. for 12 h and concentrated. The residue was purified by prep-HPLC (column: Waters Xbridge C18 150*50 mm* 10 um; mobile phase: [water (10 mM NH₄HCO₃)-ACN]; B%: 51%-81%, min) to give tert-butyl N-[2-[2-[2-[2-[2-[2-[2-[2-[2-[benzyl(methyl)amino]ethoxy]ethoxy]ethoxy]ethoxy]ethoxy]ethoxy]ethoxy]ethoxy]ethyl]-N-tert-butoxycarbonyl-carbamate (770 mg, 63% yield) as a colorless oil. ¹H NMR (400 MHz, CD₃OD): δ7.33-7.32 (m, 5H), 3.66-3.58 (m, 38H), 2.26 (s, 3H), 1.51(s, 18H). LC-MS: MS (ES′): m/z=717.3 [M+H⁺].

### 7.5 Preparation of Compound 6

To a solution of tert-butyl N-1[2-[2-[2-[2-1[2-1[2-[2-[2-1[2-[benzyl(methyl)amino]ethoxy]ethoxy]ethoxy]ethoxy]ethoxy]ethoxy]ethoxy]ethoxy]ethyl]-N-tert-butoxycarbonyl-carbamate (770 mg, 1 mmol, 1 equiv) in CF₃CH₂OH (10 mL) was added Pd(OH)₂ (150 mg, 107 μmol, 10% purity, 0.1 equiv) and the mixture was stirred at 30° C. for 12 h under H₂ (50 Psi) atmosphere. The reaction mixture was filtered and concentrated under reduced pressure to give crude tert-butyl N-tert-butoxycarbonyl-N-[2-[2-[2-[2-[2-[2-[2-[2-[2-(methylamino)ethoxy]ethoxy]ethoxy]ethoxy]ethoxy]ethoxy]ethoxy]ethoxy]ethyl]carbamate (660 mg) as a colorless oil which was used directly for next step. ¹H NMR (400 MHz, CD₃OD): δ 3.79-3.76 (m, 2H), 3.64-3.59 (m, 32H), 2.76-2.73 (m, 2H), 2.41(s, 3H), 1.51(s, 18H). LC-MS: MS (ES⁺): m/z=627.3 [M+H⁺].

### 7.6 Preparation of Compound 7

To a solution of tert-butyl N-tert-butoxycarbonyl-N-[2-[2-[2-[2-[2-[2-[2-[2-[2-(methylamino)ethoxy]ethoxy]ethoxy]ethoxy]ethoxy]ethoxy]ethoxy]ethoxy]ethyl]carbamate (560 mg, 893 μmol, 1 equiv) in DCM (3 mL) was added DIEA (173 mg, 1.3 mmol, 1.5 equiv) and methyl (E)-4-bromobut-2-enoate (160 mg, 893 μmol, 105 μL, 1 equiv) at 0° C. The mixture was stirred at 25° C. for 12 h and concentrated. The residue was purified by prep-HPLC (column: Waters Atlantis T3 150*30 mm*5 um; mobile phase: [water (10 mM NH4HCO₃)-ACN]; B%: 40%-70%, 10 min) to give methyl (E)-4-[2-[2-[2-[2-[2-[2-[2-[2-[2-[bis(tert-butoxycarbonyl) amino]ethoxy]ethoxy]ethoxy]ethoxy]ethoxy]ethoxy]ethoxy]ethoxy]ethyl-methyl-amino]but-2-enoate (340 mg, 53% yield) as a colorless oil. ¹H NMR (400 MHz, CDCl₃): δ7.06-6.99 (m, 1H), 6.13-6.09 (m, 1H), 3.88-3.62 (m, 44H), 1.52 (s, 18H). LC-MS: MS (ES⁺): m/z=725.6 [M+H⁺].

### 7.7 Preparation of Compound 8

To a solution of methyl (E)-4-[2-[2-[2-[2-[2-[2-[2-[2-[2-[bis(tert-butoxycarbonyl)amino]ethoxy]ethoxy]ethoxy]ethoxy]ethoxy]ethoxy]ethoxy]ethoxy]ethyl-methyl-amino]but-2-enoate (200 mg, 276 μmol, 1 equiv) in THF (1 mL) and H₂O (1 mL) was added LiOH.H₂O (46 mg, 1 mmol, 4 equiv). The mixture was stirred at 25° C. for 1 h and concentrated to give crude (E)-4-[[21[2-1[2-1[2-[2-1[2-1[2-1[2-1[bis(tert-butoxycarbonyl)amino]ethoxy]ethoxy]ethoxy]ethoxy]ethoxy]ethoxy]ethoxy]ethoxy]ethyl-methyl-amino]but-2-enoic acid (200 mg) as a white solid which was used directly for next step. LC-MS: MS (ES⁺): m/z=711.5 [M+H⁺].

### 7.8 Preparation of Compound 10

To a solution of (E)-4-[2-[2-[2-[2-[2-[2-[2-[2-[2-[bis(tert-butoxycarbonyl) amino]ethoxy]ethoxy]ethoxy]ethoxy]ethoxy]ethoxy]ethoxy]ethoxy]ethyl-methyl-amino]but-2-enoic acid (170 mg, 239 μmol, 1 equiv) and 4-amino-N-[4-[2-(dimethylamino)-2-oxo-ethyl]-2,3-dimethyl-phenyl]-1-[(3R)-3-piperidyl]pyrazolo [3,4-d]pyrimidine-3-carboxamide (135 mg, 299 μmol, 1.3 equiv) in DMF (1 mL) was added DIEA (93 mg, 717 μmol, 3 equiv) and HATU (100 mg, 263 μmol, 1.1 equiv). The mixture was stirred at 25° C. for 0.5 h and concentrated. The residue was purified by prep-HPLC (column: Phenomenex Gemini-NX C18 75*30 mm*3 um; mobile phase: [water (10 mM NH4HCO₃)-ACN]; B%: 34%-64%, 8 min) to give tert-butyl N-[2-[2-[2-[2-[2-[2-[2-[2-[2-[[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-methyl-amino]ethoxy]ethoxy]ethoxy]ethoxy]ethoxy]ethoxy]ethoxy]ethoxy]ethyl]-N-tert-butoxycarbonyl-carbamate (270 mg, 99% yield) as a colorless oil. ¹H NMR (400 MHz, CD₃OD): δ8.27-8.25 (m, 1H), 7.40-7.32 (m, 1H), 7.02 (d, 1H, J=8 Hz), 6.81-6.66 (m, 2H), 6.51-6.47 (m, 1H), 4.11-3.97 (m, 2H), 3.82-3.76 (m, 5H), 3.63-3.58 (m, 35H), 3.27-3.26 (m, 1H), 3.13-3.12 (m, 4H), 3.00 (s, 3H), 2.63-2.38 (m, 3H), 2.30-2.20 (m, 12H), 2.03(s, 1H), 1.50 (s, 18H). LC-MS: MS (ES⁺): m/z=1143.8 [M+H⁺].

### 7.9 Preparation of Compound 11

A mixture of tert-butyl N—[[2[2-[2-[2-[2-[2-[2-[2-[[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-methyl-amino]ethoxy]ethoxy]ethoxy]ethoxy]ethoxy]ethoxy]ethoxy]ethoxy]ethyl]-N-tert-butoxycarbonyl-carbamate (135 mg, 118 μmol, 1 equiv) in DCM (5 mL) and TFA (1 mL) was stirred at 25° C. for 0.5 h. The reaction mixture was concentrated under reduced pressure to give 4-amino-1[(3R)-1-[(E)-4-[2-[2-[2-[2-[2-[2-[2-[2-(2-aminoethoxy) ethoxy]ethoxy]ethoxy]ethoxy]ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (110 mg, TFA salt) as a colorless oil which was used directly for next step. LC-MS: MS (ES⁺): m/z=943.2 [M+H⁺].

### 7.10 Preparation of Compound I-7.

To a solution of 3-[2-[(3R)-3-methylmorpholin-4-yl]-4-(3-methylmorpholin-4-yl)pyrido[2,3-d]pyrimidin-7-yl]benzoic acid (50 mg, 111 μmol, 1 equiv), 4-amino-1-[(3R)-1-[(E)-4-[2-[2-[2-[2-[2-[2-[2-[2-(2-aminoethoxy)ethoxy]ethoxy]ethoxy]ethoxy]ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (105 mg, 99 μmol, 1 equiv, TFA salt) in DMF (2 mL) was added DIEA (43 mg, 334 μmol, 3 equiv) and HATU (47 mg, 122 μmol, 1.1 equiv). The mixture was stirred at 25° C. for 0.5 h and purified by prep-HPLC (column: Waters Xbridge 150*25 mm*5 um; mobile phase: [water (10 mM NH4HCO₃)-ACN]; B%: 33%-57%, 7 min) to give 4-amino-N-[4-[2-(dimethylamino)-2-oxo-ethyl]-2,3-dimethyl-phenyl]-1-[(3R)—1-[(E)-4-[methyl-[[2[2-[2-[21-2-[2-[2-2-[2-[[3-[2-[(3R)-3-methylmorpholin-4-yl]-4-(3-methylmorpholin-4-yl)pyrido [2,3-d]pyrimidin-7-yl]benzoyl]amino]ethoxy]ethoxy]ethoxy]ethoxy]ethoxy]ethoxy]ethoxy]ethoxy]ethyl]amino]but-2-enoyl]-3-piperidyl]pyrazolo[3,4-d]pyrimidine-3-carboxamide (50 mg, 31% yield) as a yellow solid. ¹H NMR (400 MHz, CD₃OD): δ8.61 (s, 1H), 8.36-8.21 (m, 3H), 7.95 (d, 1H, J=6.8 Hz), 7.71-7.56 (m, 2H), 7.39-7.31 (m, 1H), 7.01-6.99 (m, 1H), 6.82-6.43 (m, 2H), 4.59-4.41 (m, 3H), 4.00-3.44 (m, 55H), 3.11 (s, 3H), 2.99 (s, 3H), 2.49-2.45 (m, 2H), 2.38-2.18 (m, 13H), 1.80-1.64 (m, 1H), 1.47 (d, 3H, J=6.8 Hz), 1.34 (d, 3H, J=6.8 Hz). LC-MS: MS (ES⁺): RT=2.65 min, m/z=688.2 [M/2+H⁺], LC-MS METHOD 01.

### Example 8—Synthesis of Compond I-8 (Synthesized According to General Scheme B in a Similar Manner to Compound I-7)

4-amino-1[(3R)-1-[(E)-4-[2-[2-[2-[2-[2-[[3-[2,4-bis[(3R)-3-methylmorpholin-4-yl]pyrido[2,3-d]pyrimidin-7-yl]benzoyl]amino]ethoxy]ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (90 mg, 64% yield) as a yellow solid. ¹H NMR (400 MHz, DMSO-d₆): δ10.33-9.95 (m, 1H), 8.80-8.57 (m, 3H), 8.37-8.21 (m, 3H), 8.08 (s, 1H), 7.96 (d, 1H, J=7.6 Hz), 7.71 (d, 1H, J=8.4 Hz), 7.62 (t, 1H, J=7.6 Hz), 7.15 (s, 1H), 6.96 (d, 1H, J=8.0 Hz), 6.75-6.40 (m, 2H), 4.78 (s, 2H), 4.58-4.20 (m, 3H), 4.12-4.01 (m, 1H), 3.96-3.88 (m, 3H), 3.85-3.41 (m, 31H), 3.26-3.02 (m, 6H), 2.87 (s, 3H), 2.28-1.88 (m, 12H), 1.59 (s, 1H), 1.38 (d, 3H, J=6.8 Hz), 1.25 (d, 3H, J=6.8 Hz). LC-MS: MS (ES⁺): RT=2.456 min, m/z=1198.6 [M+H⁺], LC-MS METHOD 01.

### Example 9—Synthesis of Compound I-9 (Synthesized According to General Scheme B in a Similar Manner to Compound I-7)

4-amino-[(3R)-1-[(E)-4-[2-[2-[2-[2-[2-[2-[2-[[3-[2,4-bis[(3R)-3-methylmorpholin-4-yl]pyrido[2,3-d]pyrimidin-7-yl]benzoyl]amino]ethoxy]ethoxy]ethoxy]ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (50 mg, 42% yield) as a yellow solid. ¹H NMR (400 MHz, DMSO-d₆): δ10.27-10.01 (m, 1H), 8.73 (t, 1H, J=5.6 Hz), 8.67-8.56 (m, 2H), 8.34-8.21 (m, 3H), 8.08 (s, 1H), 7.97 (d, 1H, J=7.6 Hz), 7.71 (d, 1H, J=8.4 Hz), 7.62 (t, 1H, J=7.6 Hz), 7.16 (s, 1H), 6.96 (d, 1H, J=8.0 Hz), 6.72-6.44 (m, 2H), 4.78 (s, 2H), 4.60-4.19 (m, 3H), 4.08 (s, 1H), 3.97-3.88 (m, 3H), 3.77-3.43 (m, 37H), 3.26-2.96 (m, 8H), 2.87 (s, 3H), 2.29-1.90 (m, 12H), 1.59 (s, 1H), 1.38 (d, 3H, J=6.8 Hz), 1.25 (d, 3H, J=6.8 Hz). LC-MS: MS (ES⁺): RT=2.493 min, m/z=1286.5 [M+H⁺], LC-MS METHOD 01.

### Example 10—Synthesis of Compound I-10 (Synthesized According to General Scheme B in a Similar Manner to Compound I-7)

4-amino-N-[4-[2-(dimethylamino)-2-oxo-ethyl]-2,3-dimethyl-phenyl]-1-[(3R)-1-[(E)-4-[methyl-[[2-[2-[2-[2-[2-[2-[2-[2-[2-[2-[[3-[2-[(3R)-3-methylmorpholin-4-yl]-4-(3-methylmorpholin-4-yl)pyrido [2,3-d]pyrimidin-7-yl]benzoyl]amino]ethoxy]ethoxy]ethoxy]ethoxy]ethoxy]ethoxy]ethoxy]ethoxy]ethoxy]ethoxy]ethyl]amino]but-2-enoyl]-3-piperidyl]pyrazolo[3,4-d]pyrimidine-3-carboxamide (81 mg, 28% yield) as a yellow solid. ¹H NMR (400 MHz, MeOD): δ8.61 (s, 1H), 8.37-8.18 (m, 3H), 8.01-7.86 (m, 1H), 7.70-7.51 (m, 2H), 7.43-7.30 (m, 1H), 7.06-6.92 (m, 1H), 6.87-6.43 (m, 2H), 4.86 (s, 2H), 4.69-4.17 (m, 3H), 4.15-3.88 (m, 5H), 3.85-345 (m, 54H), 3.30-3.19 (m, 1H), 3.17-2.94 (m, 7H), 2.66-2.44 (m, 2H), 2.41-1.97 (m, 11H), 1.81-1.63 (m, 1H), 1.63-1.61 (m, 1H), 1.51-1.41 (m, 3H), 1.37-1.27 (m, 3H). LC-MS: MS (ES⁺): RT=2.481 min, m/z=732.2 [M/2+H⁺], LC-MS METHOD 10.

### Example 11—Synthesis of Compound I-11

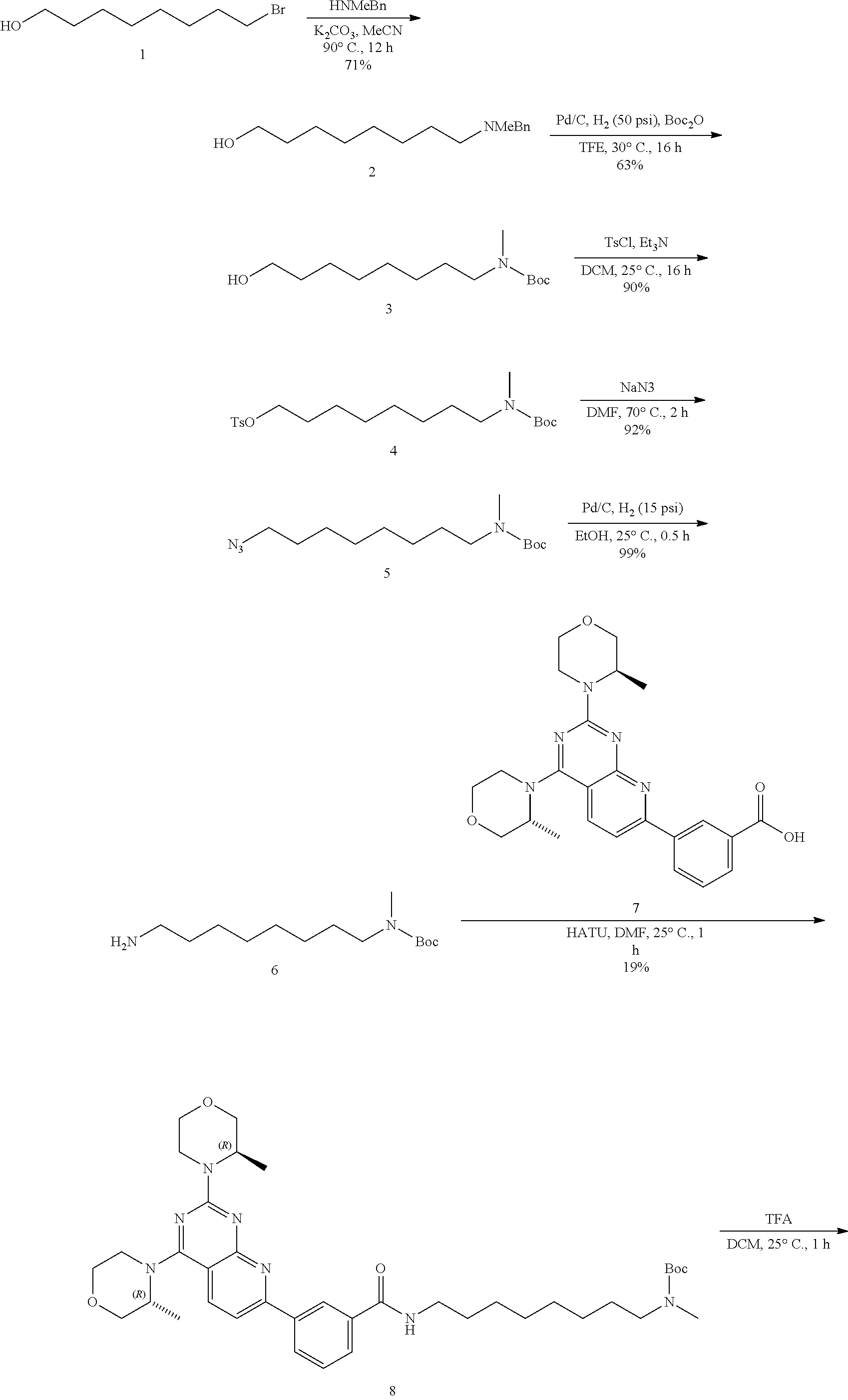

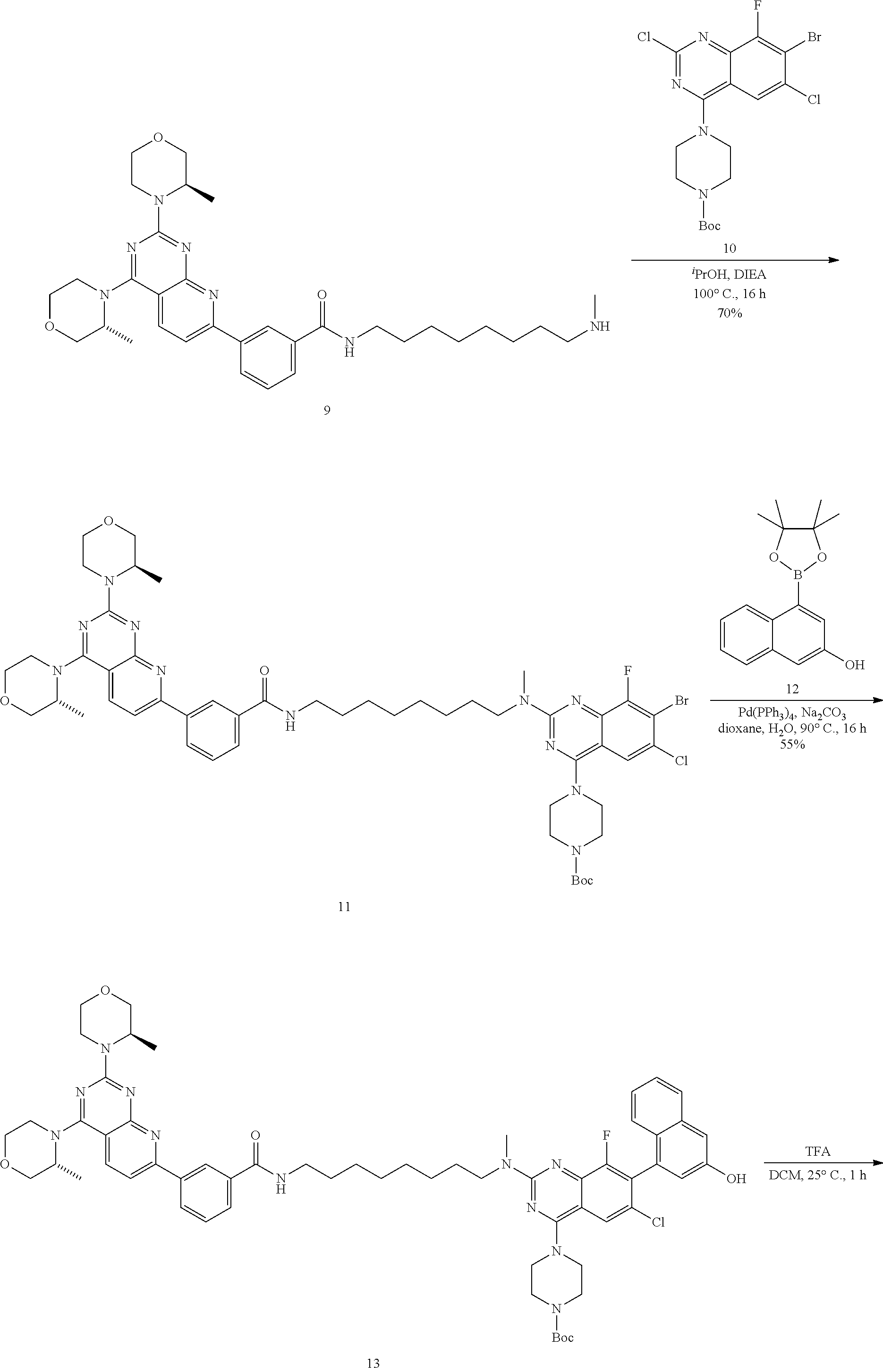

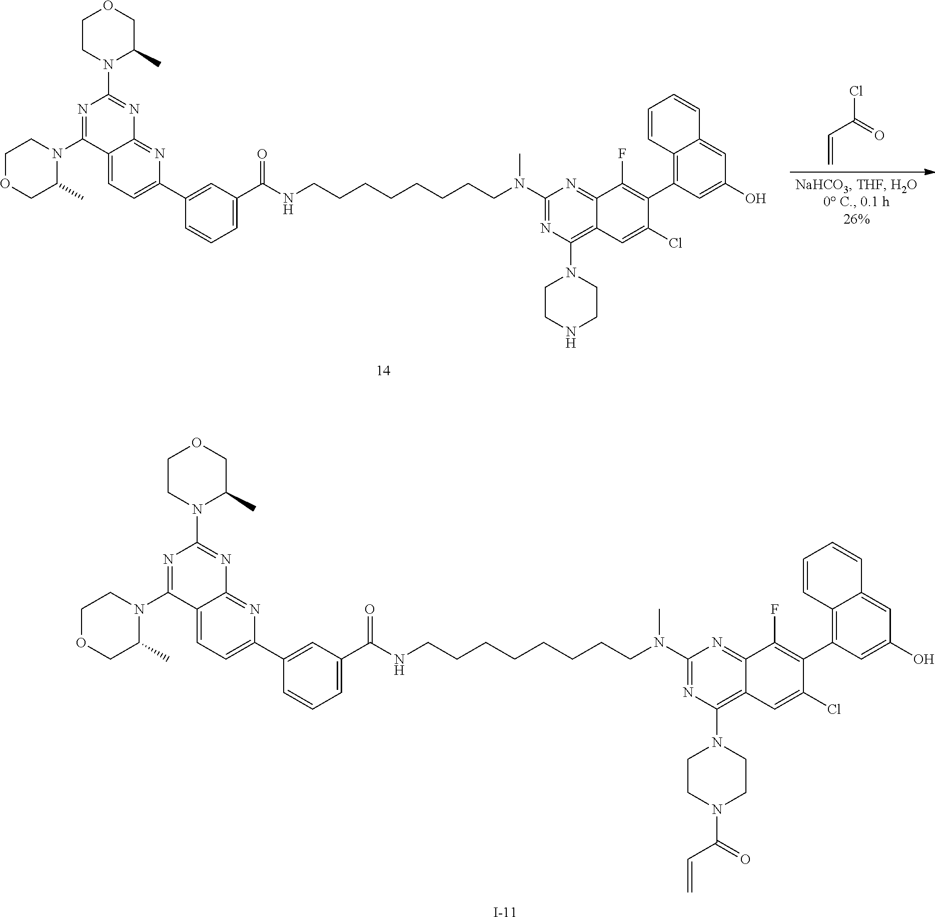

### 11.1 Preparation of Compound 2

To a solution of 8-bromooctan-1-ol (10.0 g, 47.8 mmol, 1.0 equiv) and N-methyl-1-phenyl-methanamine (5.79 g, 47.8 mmol, 1.0 equiv) in MeCN (100 mL) was added K₂CO3 (7.27 g, 52.6 mmol, 1.1 equiv). The mixture was stirred at 90° C. for 12 h. The mixture was purified by column chromatography (SiO₂, Petroleum ether: Ethyl acetate=100: 1 to 10: 1) to give 8-[benzyl(methyl)amino]octan-1-ol (8.50 g, 34.1 mmol, 71% yield) as a light yellow oil. LC-MS: MS (ES⁺): RT=0.602 min, m/z=250.5 [M+H⁺].

### 11.2 Preparation of Compound 3

To a mixture of 8-[benzyl(methyl)amino]octan-1-ol (8.50 g, 34.1 mmol, 1.0 equiv) and Boc₂O (14.9 g, 68.2 mmol, 2.0 equiv) in TFE (100 mL) was added Pd(OH)₂/C (900 mg, 10% purity) under N₂. The solution was stirred under H₂ (50 psi) at 30° C. for 16 h. The solution was filtered. The filtrate was concentrated to give a residue. The residue was purified by column chromatography (SiO₂, Petroleum ether: Ethyl acetate=50: 1 to 5: 1) to give the desired product tert-butyl N-(8-hydroxyoctyl)-N-methyl-carbamate (6.00 g, 21.5 mmol, 63% yield, 93% purity) as a colorless oil. LC-MS: MS (ES⁺): RT=0.732 min, m/z=282.0 [M+Na+].

### 11.3 Preparation of Compound 4

To a mixture of tert-butyl N-(8-hydroxyoctyl)-N-methyl-carbamate (2.00 g, 7.71 mmol, 1.0 equiv) and TEA (2.34 g, 23.1 mmol, 3.0 equiv) in DCM (30 mL) were added 4-methylbenzenesulfonyl chloride (2.94 g, 15.4 mmol, 2.0 equiv) at 0° C. The mixture was stirred 25° C. for 16 h. The solution was concentrated to give a residue. The residue was purified by column chromatography (SiO₂, Petroleum ether: Ethyl acetate=50: 1 to 2: 1) to give the desired product 8-[tert-butoxycarbonyl(methyl)amino]octyl 4-methylbenzenesulfonate (3.00 g, 6.96 mmol, 90% yield) as a colorless oil. LC-MS: MS (ES⁺): RT=0.895 min, m/z=414.1 [M+H⁺]. ¹H NMR (400 MHz, CDCl₃): δ7.79 (d, J=8.4 Hz, 2H), 7.34 (d, J=8.0 Hz, 2H), 4.01 (t, J=6.8 Hz, 2H), 3.17 (t, J=7.2 Hz, 2H), 2.82 (s, 3H), 2.45 (s, 3H), 1.68-1.58 (m, 2H), 1.49-1.42 (m, 11H), 1.24 (s, 8H).

### 11.4 Preparation of Compound 5

To a mixture of 8-[tert-butoxycarbonyl(methyl)amino]octyl 4-methylbenzenesulfonate (3.00 g, 7.25 mmol, 1.0 equiv) in DMF (15 mL) was added NaN₃ (1.47 g, 22.6 mmol, 3.1 equiv). The mixture was stirred at 70° C. for 2 h. To the reaction mixture was added water (50 mL). The mixture was extracted with EtOAc (30×3 mL). The combined organic phase was washed with brine (50 mL×3), dried over by anhydrous Na₂SO₄ and filtered. The filtrate was concentrated to give tert-butyl-N-(8-azidooctyl)-N-methyl-carbamate (1.90 g, 6.68 mmol, 92% yield) as a colorless oil. ¹H NMR (400 MHz, CDCl₃): δ3.26 (t, J=7.2 Hz, 2H), 3.19 (t, J=6.4 Hz, 2H), 2.83 (s, 3H), 1.62-1.44 (m, 13H), 1.39-1.24 (m, 8H).

### 11.5 Preparation of Compound 6

To a mixture of tert-butyl N-(8-azidooctyl)-N-methyl-carbamate (1.00 g, 3.52 mmol, 1.0 equiv) in EtOH (20 mL) was added Pd/C (100 mg, 10% purity) under N₂. The solution was stirred under H₂ (15 psi, balloon) at 25° C. for 30 min. The mixture was filtered. The filtrate was concentrated to give crude product tert-butyl N-(8-aminooctyl)-N-methyl-carbamate (900 mg, 3.48 mmol, 99% yield) as a colorless oil. ¹H NMR (400 MHz, CDCl₃): δ 3.21-3.03 (m, 2H), 2.76 (s, 3H), 2.64-2.21 (m, 2H), 1.63-1.31 (m, 15H), 1.31-1.06 (m, 8H).

### 11.6 Preparation of Compound 8

To a mixture of 3-[2,4-bis[(3R)-3-methylmorpholin-4-yl]pyrido[2,3-d]pyrimidin-7-yl]benzoic acid (400 mg, 890 μmol, 1.0 equiv) and tert-butyl N-(8-aminooctyl)-N-methyl-carbamate (230 mg, 890 μmol, 1.0 equiv) in DMF (2.0 mL) and DCM (2.0 mL) were added DIEA (345 mg, 2.67 mmol, 3.0 equiv) and HATU (406 mg, 1.07 mmol, 1.2 equiv). The mixture was stirred at 25° C. for 1 h. The mixture was purified by prep-HPLC (column: Phenomenex luna C18 150*40 mm* 15 um; mobile phase: [water(0.225% FA)-ACN]; B%: 28% 58%, 10 min) to give tert-butyl-N-[8-[[3-[2,4-bis[(3R)-3-methylmorpholin-4-yl]pyrido[2,3-d]pyrimidin-7-yl]benzoyl]amino]octyl]-N-methyl-carbamate (124 mg, 173 μmol, 19% yield) as a yellow gum. LC-MS: MS (ES⁺): RT=0.675 min, m/z=690.4 [M+H⁺].

### 11.7 Preparation of Compound 9

To a mixture of tert-butyl-N-[8-[[3-[2,4-bis[(3R)-3-methylmorpholin-4-yl]pyrido[2,3-d]pyrimidin-7-yl]-benzoyl]amino]octyl]-N-methyl-carbamate (124 mg, 180 mol, 1.0 equiv) in DCM (2 mL) was added TFA (1.54 g, 13.5 mmol, 1 mL, 75.1 equiv). The mixture was stirred at 25° C. for 1 h. The mixture was concentrated to give 3-[2,4-bis[(3R)-3-methylmorpholin-4-yl]pyrido[2,3-d]-pyrimidin-7-yl]-N-[8-(methylamino)octyl]benzamide (126 mg, crude, TFA salt) as a light yellow gum. LC-MS: MS (ES⁺): RT=0.775 min, m/z=590.4 [M+H⁺].

### 11.8 Preparation of Compound 11

To a mixture of 3-[2,4-bis[(3R)-3-methylmorpholin-4-yl]pyrido[2,3-d]pyrimidin-7-yl]-N-[8-(methylamino)octyl]benzamide (126 mg, 179 μmol, 1.0 equiv, TFA salt) and tert-butyl-4-(7-bromo-2,6-dichloro-8-fluoro-quinazolin-4-yl)piperazine-1-carboxylate (86 mg, 0.18 mmol, 1.0 equiv) in i-PrOH (4 mL) was added DIEA (116 mg, 895 μmol, 5.0 equiv). The mixture was stirred at 100° C. for 16 h. The mixture was purified by prep-TLC (SiO₂, DCM: MeOH=10: 1) to give tert-butyl-4-[2-[8-[[3-[2,4-bis[(3R)-3-methylmorpholin-4-yl]pyrido[2,3-d]pyrimidin-7-yl]benzoyl]amino]octyl-methyl-amino]-7-bromo-6-chloro-8-fluoro-quinazolin-4-yl]piperazine-1-carboxylate (130 mg, 126 μmol, 70% yield) as a yellow gum. LC-MS: MS (ES⁺): RT=0.964 min, m/z=1034.4 [M+H⁺].

### 11.9 Preparation of Compound 13

To a solution of tert-butyl-4-[2-[8-[[3-[2,4-bis[(3R)-3-methylmorpholin-4-yl]pyrido[2,3-d]pyrimidin-7-yl]benzoyl]amino]octylmethyl-amino]-7-bromo-6-chloro-8-fluoro-quinazolin-4-yl]piperazine-1-carboxylate (120 mg, 116 μmol, 1.0 equiv), 4-(4,4,5,5-tetramethyl-1,3,2-dioxaborolan-2-yl)naphthalen-2-ol (62 mg, 0.23 mmol, 2.0 equiv) and Na₂CO₃ (30.8 mg, 290 μmol, 2.5 equiv) in dioxane (3 mL) and H₂O (0.6 mL) was added Pd(PPh₃)₄(26.8 mg, 23.2 μmol, 0.2 equiv). The mixture solution was heated to 90° C. and stirred under N₂ for 4 h. The mixture was purified by prep-TLC (Dichloromethane: Methanol=10: 1) to give tert-butyl-4-[2-[8-[[3-[2,4-bis[(3R)-3-methylmorpholin-4-yl]pyrido[2,3-d]pyrimidin-7-yl]benzoyl]-amino]-octyl-methyl-amino]-6-chloro-8-fluoro-7-(3-hydroxy-1-naphthyl)quinazolin-4-yl]piperazine-1-carboxylate (70 mg, 64 μmol, 55% yield) as a yellow oil. LC-MS: MS (ES⁺): RT=0.848 min, m/z=1095.8 [M+H⁺].

### 11.10 Preparation of Compound 14

To a solution of tert-butyl-4-[2-[8-[[3-[2,4-bis[(3R)-3-methylmorpholin-4-yl]pyrido[2,3-d]pyrimidin-7-yl]benzoyl]amino]octylmethyl-amino]-6-chloro-8-fluoro-7-(3-hydroxy-1-naphthyl)quinazolin-4-yl]piperazine-1-carboxylate (70 mg, 64 μmol, 1.0 equiv) in DCM (2 mL) was added TFA (1.54 g, 13.5 mmol, 211 equiv). The mixture solution was stirred at 25° C. for 1 h. The solution was concentrated to give the crude product 3-[2,4-bis[(3R)-3-methylmorpholin-4-yl]pyrido[2,3-d]pyrimidin-7-yl]-N-[8-[[6-chloro-8-fluoro-7-(3-hydroxy-1-naphthyl)-4-piperazin-1-yl-quinazolin-2-yl]-methyl-amino]octyl]benzamide (70 mg, 63 μmol, 99% yield, TFA salt) as a yellow oil. LC-MS: MS (ES⁺): m/z=995.8 [M+H⁺].

### 11.11 Preparation of Compound I-11

To a solution of 3-[2,4-bis[(3R)-3-methylmorpholin-4-yl]pyrido[2,3-d]pyrimidin-7-yl]-N-[8-[[6-chloro-8-fluoro-7-(3-hydroxy-1-naphthyl)-4-piperazin-1-yl-quinazolin-2-yl]-methyl-amino]octyl]benzamide (70 mg, 70 μmol, 1.0 equiv) in NaHCO₃ (1 M, aq., 0.6 mL, 8.5 equiv) and THF (3.0 mL) was added prop-2-enoyl chloride (6.4 mg, 70 μmol, 1.0 equiv) at 0° C. The mixture solution was stirred at 0° C. for 0.1 h. The mixture was purified by prep-HPLC(column: Unisil 3-100 C18 Ultra 150*50 mm*3 um; mobile phase: [water(0.225% FA)-ACN]; B%: 30%-60%, 10 min) to give the desired product 3-[2,4-bis[(3R)-3-methylmorpholin-4-yl]pyrido[2,3-d]pyrimidin-7-yl]-N-[8-[[6-chloro-8-fluoro-7-(3-hydroxy-1-naphthyl)-4-(4-prop-2-enoylpiperazin-1-yl)quinazolin-2-yl]-methyl-amino]octyl]benzamide (19 mg, 18 μmol, 26% yield) as a yellow solid. ¹H NMR (400 MHz, MeOD): δ8.59 (s, 1H), 8.29-8.21 (m, 2H), 7.92 (d, J=8.0 Hz, 1H), 7.75-7.69 (m, 2H), 7.64-7.57 (m, 2H), 7.41-7.34 (m, 1H), 7.28-7.22 (m, 2H), 7.20-7.14 (m, 1H), 7.02 (d, J=2.4 Hz, 1H), 6.83-6.73 (m, 1H), 6.24 (dd, J=16.4, 1.8 Hz, 1H), 5.77 (dd, J=10.8, 1.8 Hz, 1H), 4.55-4.44 (m, 2H), 4.01-3.91 (m, 3H), 3.91-3.62 (m, 17H), 3.58-3.48 (m, 1H), 3.41-3.35 (m, 2H), 3.18 (s, 3H), 1.73-1.56 (m, 4H), 1.49-1.28 (m, 15H). LC-MS: MS (ES⁺): RT=2.910 min, m/z=1051.4 [M+H⁺], LC-MS METHOD 01.

### Example 12—Synthesis of Compound I-20 (Synthesized According to General Scheme C)

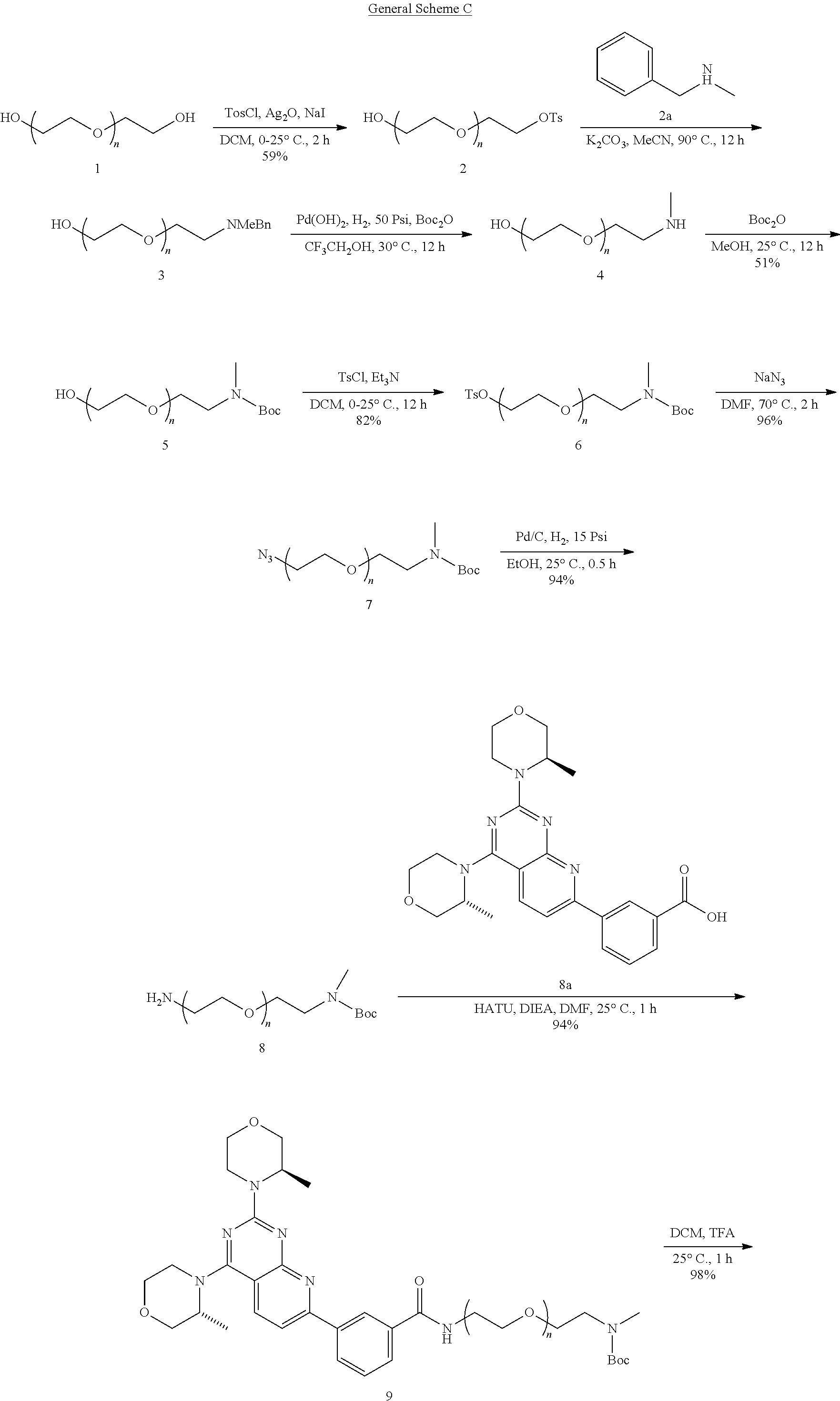

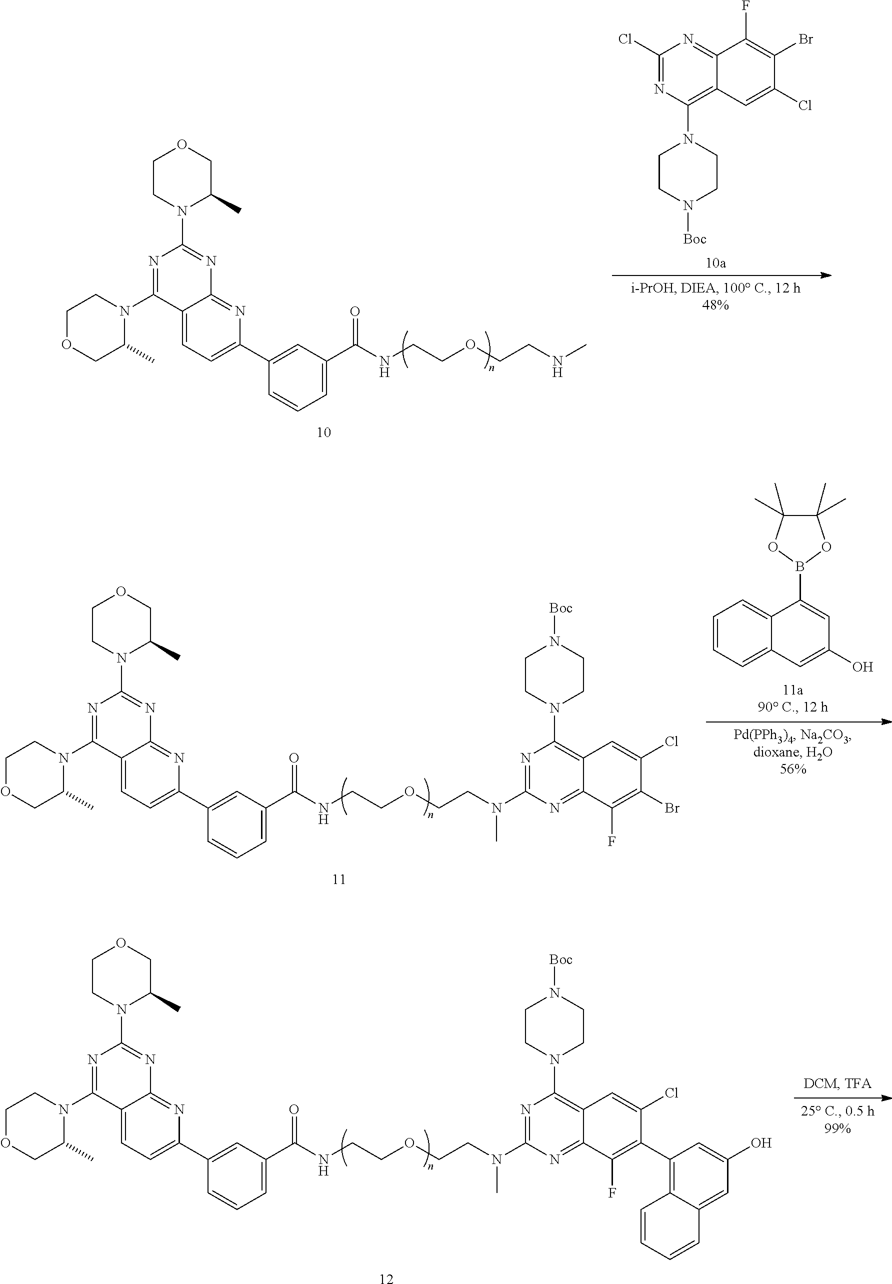

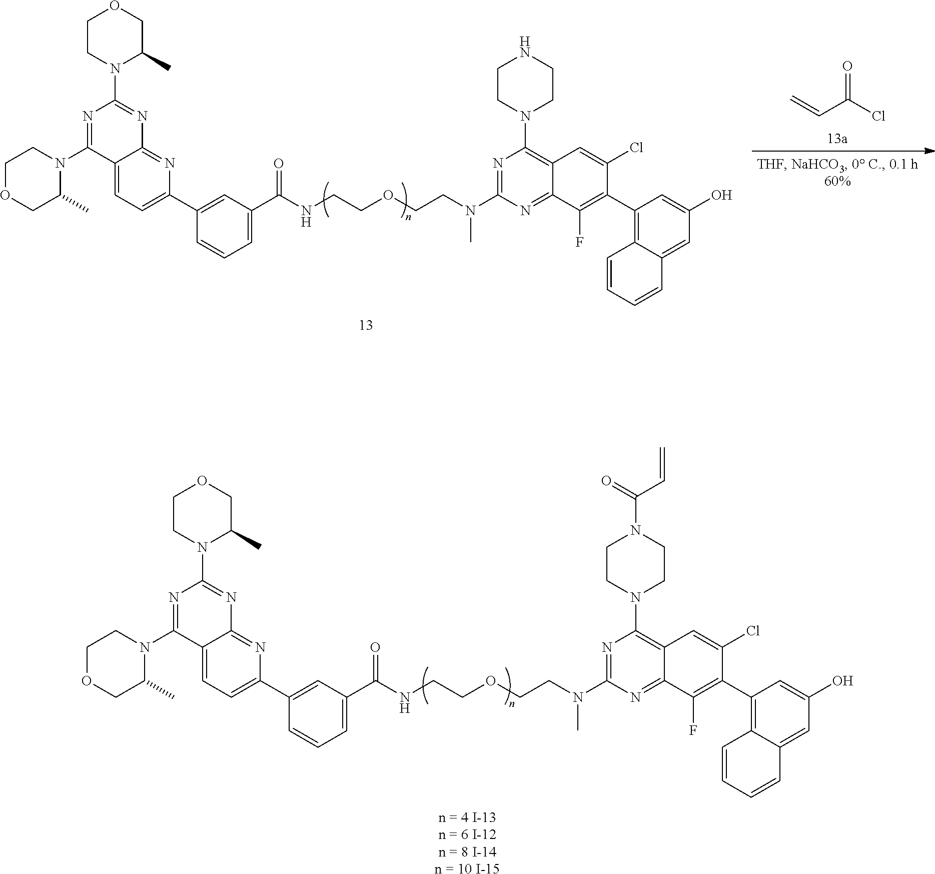

### 12.1 Preparation of Compound 2

To a stirred solution of 2-[2-[2-[2-[2-[2-(2-hydroxyethoxy)ethoxy]ethoxy]ethoxy]ethoxy]ethoxy]ethanol (10.0 g, 30.6 mmol, 1.0 equiv) in DCM (200 mL) at 0° C. were added Ag₂0 (10.7 g, 46.0 mmol, 1.5 equiv), NaI (5.05 g, 33.7 mmol, 1.1 equiv) and TosCl (6.13 g, 32.2 mmol, 1.1 equiv). The reaction mixture was stirred at 25° C. for 12 h. The mixture was filtered and concentrated to give a residue. The residue was purified by silica column chromatography on silica gel (Dichloromethane: Methanol=100/1 to 50/1) to give the desired product 2-[2-[2-[2-[2-[2-(2-hydroxyethoxy)ethoxy]ethoxy]ethoxy]ethoxy]ethoxy]ethyl 4-methylbenzenesulfonate (17.5 g, 36.4 mmol, 59% yield) as a colorless oil. LC-MS: MS (ES⁺): RT=0.571 min, m/z=481.1 [M+H⁺].

### 12.2 Preparation of Compound 3

To a solution of 2-[2-[2-[2-[2-[2-(2-hydroxyethoxy)ethoxy]ethoxy]ethoxy]ethoxy]ethoxy]ethyl 4-methylbenzenesulfonate (17.5 g, 36.4 mmol, 1.0 equiv) in MeCN (150 mL) were added K₂CO₃ (7.55 g, 54.6 mmol, 1.5 equiv) and N-methyl-1-phenyl-methanamine (4.41 g, 36.4 mmol, 4.7 mL, 1.0 equiv). The mixture was stirred at 90° C. for 12 h. The mixture was filtered and concentrated to give a residue. The residue was purified by silica column chromatography on silica gel (Dichloromethane: Methanol from 100/1 to 10/1) to give the crude product 2-[2-[2-[2-[2-[2-[2-[benzyl(methyl)amino]ethoxy]ethoxy]ethoxy]ethoxy]ethoxy]ethoxy]ethanol (20.0 g, crude) as a light yellow oil. LC-MS: MS (ES⁺): RT=0.842 min, m/z=430.1 [M+H⁺].

### 12.3 Preparation of Compound 4

To a solution of 2-[2-[2-1[2-1[2-[2-1[2-[benzyl(methyl)amino]ethoxy]ethoxy]ethoxy]ethoxy]ethoxy]ethoxy]ethanol (10.0 g, 23.3 mmol, 1.0 equiv) in CF₃CH₂OH (120 mL) were added Pd(OH)₂/C (1.50 g, 10% purity) and Boc₂0 (10.2 g, 46.6 mmol, 10.7 mL, 2.0 equiv) under N₂. The suspension was degassed under vacuum and purged with H₂ several times. The mixture was stirred under H₂ (50 psi) at 30° C. for 12 h. The mixture was filtered and concentrated to give the crude product 2-[2-[2-[2-[2-[2-[2-(methylamino)ethoxy]ethoxy]ethoxy]ethoxy]ethoxy]ethoxy]ethanol (15.0 g, crude) as a colorless oil. LC-MS: MS (ES⁺): RT=0.900 min, m/z=340.0 [M+H⁺].

### 12.4 Preparation of Compound 5

To a solution of 2-[2-[2-[2-[2-[2-[2-(methylamino)ethoxy]ethoxy]ethoxy]ethoxy]ethoxy]ethoxy]ethanol (15.0 g, 44.2 mmol, 1.0 equiv) in MeOH (200 mL) were added Boc₂0 (9.64 g, 44.2 mmol, 1.0 equiv). The mixture was stirred at 25° C. for 12 h. The mixture was concentrated to give a residue. The residue was purified by silica column chromatography on silica gel (Dichloromethane: Methanol=100/1 to 20/1) to give the desired product tert-butyl N-[2-[2-[2-[2-[2-[2-(2-hydroxyethoxy)ethoxy]ethoxy]ethoxy]-ethoxy]ethoxy]ethyl]-N-methyl-carbamate (10.0 g, 22.8 mmol, 51% yield) as a colorless oil.LC-MS: MS (ES⁺): RT=0.998 min, m/z=457.2 [M+18+].

### 12.5 Preparation of Compound 6

To a solution of tert-butyl N—[[2[2-[2-[2-[2-(2-hydroxyethoxy)ethoxy]ethoxy]ethoxy]ethoxy]ethoxy]-ethyl]-N-methylcarbamate (3.00 g, 6.83 mmol, 1.0 equiv) in DCM (50 mL) were added TEA (1.38 g, 13.7 mmol, 1.9 mL, 2.0 equiv) and TosCl (1.95 g, 10.2 mmol, 1.5 equiv) at 0° C. The mixture was stirred at 25° C. for 12 h. The mixture was concentrated to give a residue. The residue was purified by silica column chromatography on silica gel (Dichloromethane: Methanol=100/1 to 20/1) to give the desired product 2-[2-[2-[2-[2-[2-[2-[tert-butoxycarbonyl(methyl)amino]ethoxy]ethoxy]ethoxy]ethoxy]ethoxy]-ethoxy]ethyl 4-methylbenzenesulfonate (3.34 g, 5.63 mmol, 82% yield) as a light yellow oil.

### 12.6 Preparation of Compound 7

To a solution of 2-[2-[2-[2-[2-[2-[2-[tert-butoxycarbonyl(methyl)amino]ethoxy]ethoxy]ethoxy]ethoxy]-ethoxy]ethoxy]ethyl 4-methylbenzenesulfonate (3.34 g, 5.63 mmol, 1.0 equiv) in DMF (20 mL) was added NaN₃ (1.17 g, 18.0 mmol, 3.2 equiv). The mixture was stirred at 70° C. for 2 h. To the reaction mixture was added water (50 mL) and the mixture was extracted with ethyl acetate (3×50 mL). The combined organic phase was washed with brine (3×50 mL), dried over by Na₂SO₄ and filtered. The filtrate was concentrated to give the desired product tert-butylN-[2-[2-[2-[2-[2-[2-(2-azidoethoxy)ethoxy]ethoxy]-ethoxy]ethoxy]ethoxy]ethyl]-N-methyl-carbamate (2.50 g, 5.38 mmol, 96% yield) as a colorless oil. ¹H NMR (400 MHz, CDCl₃): δ3.71-3.63 (m, 24H), 3.40 (d, J=5.1 Hz, 4H), 2.91 (s, 3H), 1.45 (s, 9H). LC-MS: MS (ES′): RT=0.900 min, m/z=465.1 [M+H⁺].

### 12.7 Preparation of Compound 8

To a solution of tert-butyl N-[2-[2-[2-[2-[2-[2-(2-azidoethoxy)ethoxy]ethoxy]ethoxy]ethoxy]ethoxy]-ethyl]-N-methyl-carbamate (1.30 g, 2.80 mmol, 1.0 equiv) in EtOH (20 mL) was added Pd/C (0.50 g, 10% purity). The mixture solution was stirred under H₂ (15 psi, balloon) for 0.5 h at 25° C. The mixture was filtered and concentrated to give the desired product tert-butyl N—[[2[2-[2-[2-[2-(2-aminoethoxy)ethoxy]ethoxy]ethoxy]ethoxy]ethoxy]ethyl]-N-methyl-carbamate (1.15 g, 2.62 mmol, 94% yield) as a colorless oil. ¹H NMR (400 MHz, CDCl₃): δ3.70-3.60 (m, 24H), 3.38 (s, 2H), 2.94-2.88 (m, 3H), 2.88-2.85 (m, 1H), 2.80 (t, J=5.4 Hz, 1H), 1.45 (s, 9H).

### 12.8 Preparation of Compound 9

To a solution of tert-butyl N-[2-[2-[2-[2-[2-[2-(2-aminoethoxy)ethoxy]ethoxy]ethoxy]ethoxy]ethoxy]-ethyl]-N-methyl-carbamate (293 mg, 667 μmol, 1.0 equiv) in DMF (6 mL) were added HATU (305 mg, 801 μmol, 1.2 equiv), 3-[2,4-bis[(3R)-3-methylmorpholin-4-yl]pyrido[2,3-d]pyrimidin-7-yl]benzoic acid (300 mg, 667 μmol, 1.0 equiv) and DIEA (173 mg, 1.33 mmol, 0.2 mL, 2.0 equiv). The mixture was stirred at 25° C. for 1 h. The solution was purified by prep-HPLC (column: Phenomenex luna C18 150*40 mm*15 um; mobile phase: [water (0.1% TFA)-ACN]; B%: 26%-56%, 11 min) to give the desired product tert-butyl N-[2-[2-[2-[2-[2-[2-[2-[[3-[2,4-bis[(3R)-3-methylmorpholin-4-yl]pyrido[2,3-d]pyrimidin-7-yl]-benzoyl]amino]ethoxy]ethoxy]ethoxy]ethoxy]ethoxy]ethoxy]ethyl]-N-methyl-carbamate (195 mg, 224 μmol, 34% yield) as a yellow oil. ¹H NMR (400 MHz, DMSO-d₆): δ8.80-8.66 (m, 2H), 8.46 (d, J=8.8 Hz, 1H), 8.36 (d, J=7.6 Hz, 1H), 8.10-7.96 (m, 2H), 7.69 (t, J=7.6 Hz, 1H), 4.84-4.67 (m, 2H), 4.38 (d, J=16.8 Hz, 2H), 4.03-3.91 (m, 2H), 3.87-3.61 (m, 20H), 3.60-3.51 (m, 10H), 3.41 (s, 4H), 3.32-3.26 (m, 2H), 2.78 (s, 3H), 1.52 (d, J=7.2 Hz, 3H), 1.37 (s, 9H), 1.34 (d, J=6.8 Hz, 3H). LC-MS: MS (ES⁺): RT=1.017 min, m/z=870.5 [M+H⁺].

### 12.9 Preparation of Compound 10

To a solution of tert-butyl N-[2-[2-[2-[2-[2-[2-[2-[[3-[2,4-bis[(3R)-3-methylmorpholin-4-yl]pyrido[2,3-d]-pyrimidin-7-yl]benzoyl]amino]ethoxy]ethoxy]ethoxy]ethoxy]ethoxy]ethoxy]ethyl]-N-methyl-carbamate (145 mg, 167 μmol, 1.0 equiv) in DCM (2 mL) was added TFA (616 mg, 5.40 mmol, 0.4 mL, 32.4 equiv). The mixture was stirred at 25° C. for 1 h. The mixture was concentrated to give the desired product 3-[2,4-bis[(3R)-3-methylmorpholin-4-yl]pyrido[2,3-d]pyrimidin-7-yl]-N-[2-[2-[2-[2-[2-[2-[2-(methylamino) ethoxy]ethoxy]ethoxy]ethoxy]ethoxy]ethoxy]ethyl]benzamide (145 mg, 164 μmol, 98% yield, TFA salt) as a yellow oil. LC-MS: MS (ES⁺): RT=0.914 min, m/z=770.4 [M+H⁺].

### 12.10 Preparation of Compound 11

To a solution of 3-[2,4-bis[(3R)-3-methylmorpholin-4-yl]pyrido[2,3-d]pyrimidin-7-yl]-N-[2-[2-[2-[2-[2-[2-[2-(methylamino)ethoxy]ethoxy]ethoxy]ethoxy]ethoxy]ethoxy]ethyl]benzamide (145 mg, 188 μmol, 1.0 equiv) in i-PrOH (3 mL) were added DIEA (122 mg, 942 μmol, 5.0 equiv) and tert-butyl 4-(7-bromo-2,6-dichloro-8-fluoro-quinazolin-4-yl)piperazine-1-carboxylate (90 mg, 0.19 mmol, 1.0 equiv). The mixture was stirred at 100° C. for 12 h. The mixture was concentrated to give a residue. The residue was purified by prep-TLC (Dichloromethane: Methanol=10/1) to give the desired product tert-butyl 4-[2-[2-[2-[2-[2-[2-[2-[2-[[3-[2,4-bis[(3R)-3-methylmorpholin-4-yl]pyrido[2,3-d]pyrimidin-7-yl]benzoyl]amino]ethoxy]ethoxy]ethoxy]ethoxy]ethoxy]ethoxy]ethyl-methyl-amino]-7-bromo-6-chloro-8-fluoro-quinazolin-4-yl]piperazine-1-carboxylate (110 mg, 90.6 μmol, 48% yield) as a yellow oil. LC-MS: MS (ES⁺): RT=0.667 min, m/z=1214.4 [M+H⁺].

### 12.11 Preparation of Compound 12

to a solution of tert-butyl 4-[2-[2-[2-[2-[2-[2-[2-[2-[[3-[2,4-bis[(3R)-3-methylmorpholin-4-yl]pyrido[2,3-d]pyrimidin-7-yl]benzoyl]amino]ethoxy]ethoxy]ethoxy]ethoxy]ethoxy]ethoxy]ethyl-methyl-amino]-7-bromo-6-chloro-8-fluoro-quinazolin-4-yl]piperazine-1-carboxylate (110 mg, 90.6 μmol, 1.0 equiv), 4-(4,4,5,5-tetramethyl-1,3,2-dioxaborolan-2-yl)naphthalen-2-ol (49 mg, 0.18 mmol, 2.0 equiv), Na₂CO₃ (24 mg, 0.23 μmol, 2.5 equiv) in dioxane (3 mL) and H₂O (0.6 mL) was added Pd(PPh₃)₄(21 mg, 0.018 mmol, 0.2 equiv). The mixture solution was stirred at 90° C. under N₂ for 12 h. The mixture was filtered and concentrated to give a residue. The residue was purified by prep-TLC (Dichloromethane: Methanol=10/1) to give the desired product tert-butyl 4-[2-[2-[2-[2-[2-[2-[2-[2-[[3-[2,4-bis[(3R)-3-methylmorpholin-4-yl]pyrido[2,3-d]pyrimidin-7-yl]benzoyl]amino]ethoxy]ethoxy]ethoxy]ethoxy]ethoxy]ethoxy]ethyl-methyl-amino]-6-chloro-8-fluoro-7-(3-hydroxy-1-naphthyl)quinazolin-4-yl]piperazine-1-carboxylate (65 mg, 51 μmol, 56% yield) as a yellow oil. LC-MS: MS (ES⁺): RT=0.650 min, m/z=1277.6 [M+H⁺].

### 12.12 Preparation of Compound 13

To a solution of tert-butyl 4-[2-[2-[2-[2-[2-[2-[2-[2-[[3-[2,4-bis[(3R)-3-methylmorpholin-4-yl]pyrido[2,3-d]pyrimidin-7-yl]benzoyl]amino]ethoxy]ethoxy]ethoxy]ethoxy]ethoxy]ethoxy]ethyl-methyl-amino]-6-chloro-8-fluoro-7-(3-hydroxy-1-naphthyl)quinazolin-4-yl]piperazine-1-carboxylate (65 mg, 51 μmol, 1.0 equiv) in DCM (0.5 mL) was added TFA (154 mg, 1.4 mmol, 0.1 mL, 26.5 equiv). The mixture was stirred at 25° C. for 0.5 h. The mixture was concentrated to give the desired product 3-[2,4-bis[(3R)-3-methylmorpholin-4-yl]pyrido [2,3-d]pyrimidin-7-yl]-N-[2-[2-[2-[2-[2-[2-[2-[[6-chloro-8-fluoro-7-(3-hydroxy-1-naphthyl)-4-piperazin-1-yl-quinazolin-2-yl]-methyl-amino]ethoxy]ethoxy]ethoxy]ethoxy]ethoxy]ethoxy]ethyl]benzamide (65 mg, 50 μmol, 99% yield, TFA salt) as a yellow oil. LC-MS: MS (ES⁺): RT=0.558 min, m/z=1176.4 [M+H⁺].

### 12.13 Preparation of Compound I-12

To a solution of 3-[2,4-bis[(3R)-3-methylmorpholin-4-yl]pyrido[2,3-d]pyrimidin-7-yl]-N-[2-[2-[2-[2-[2-[2-[2-[[6-chloro-8-fluoro-7-(3-hydroxy-1-naphthyl)-4-piperazin-1-yl-quinazolin-2-yl]-methylamino]ethoxy]ethoxy]ethoxy]ethoxy]ethoxy]ethoxy]ethyl]benzamide (65 mg, 50 μmol, 1.0 equiv, TFA salt) in NaHCO₃ (1 M, 0.6 mL, 11.9 equiv) and THF (3 mL) was added prop-2-enoyl chloride (4.6 mg, 50 μmol, 1.0 equiv) at 0° C. The mixture solution wa stirred at 0° C. for 0.1 h. The mixture was adjusted pH to 6 With AcOH, diluted with water (10 mL) and extracted with DCM (3×15 mL). The combined organic layers were washed with brine (20 mL), dried over by sodium sulfate and filtered. The filtrate was concentrated under reduced pressure to give a residue. The residue was purified by prep-HPLC (column: Unisil 3-100 Cis Ultra 150*50 mm*3 um; mobile phase: [water (0.225% FA)-ACN]; B%: 18%-48%, 10 min) to give the desired product 3-[2,4-bis[(3R)-3-methylmorpholin-4-yl]pyrido[2,3-d]pyrimidin-7-yl]-N—[[21[2-[2-[2-[2-[12-[[6-chloro-8-fluoro-7-(3-hydroxy-1-naphthyl)-4-(4-prop-2-enoylpiperazin-1-yl)quinazolin-2-yl]-methylamino]ethoxy]ethoxy]ethoxy]ethoxy]ethoxy]ethoxy]ethyl]benzamide (37 mg, 30 μmol, 60% yield, 100% purity) as a yellow solid. ¹H NMR (400 MHz, DMSO-d₆): δ10.00 (s, 1H), 8.72 (t, J=5.4 Hz, 1H), 8.64 (s, 1H), 8.29 (d, J=7.8 Hz, 1H), 8.22 (d, J=8.4 Hz, 1H), 7.96 (d, J=7.8 Hz, 1H), 7.82-7.75 (m, 2H), 7.69 (d, J=8.4 Hz, 1H), 7.60 (t, J=7.8 Hz, 1H), 7.42 (td, J=4.0, 8.4 Hz, 1H), 7.26 (d, J=2.4 Hz, 1H), 7.21 (d, J=4.0 Hz, 2H), 7.03 (d, J=2.4 Hz, 1H), 6.83 (dd, J=10.4, 16.8 Hz, 1H), 6.16 (dd, J=2.4, 16.6 Hz, 1H), 5.84-5.67 (m, 1H), 4.76 (s, 1H), 4.42 (d, J=13.8 Hz, 2H), 3.94-3.86 (m, 3H), 3.84-3.71 (m, 12H), 3.67-3.59 (m, 6H), 3.56-3.50 (m, 8H), 3.49-3.40 (m, 18H), 3.19 (s, 3H), 1.37 (d, J=6.8 Hz, 3H), 1.24 (d, J=6.8 Hz, 3H). LC-MS: MS (ES⁺): RT=2.006 min, m/z=1231.5 [M+H⁺], LC-MS METHOD 25.

### Example 13—Synthesis of Compound I-13 (Synthesized According to General Scheme C in a Similar Manner to Compound I-12)

3-[2,4-bis[(3R)-3-methylmorpholin-4-yl]pyrido[2,3-d]pyrimidin-7-yl]-N-[2-[2-[2-[2-[2-[ [6-chloro-8-fluoro-7-(3-hydroxy-1-naphthyl)-4-(4-prop-2-enoylpiperazin-1-yl)quinazolin-2-yl]-methyl-amino]ethoxy]ethoxy]ethoxy]ethoxy]ethyl]benzamide (32 mg, 26 μmol, 46% yield, 95% purity) as a yellow solid. ¹H NMR (400 MHz, METHANOL-d₄): δ8.57 (s, 1H), 8.22 (d, J=7.8 Hz, 1H), 8.15 (d, J=8.4 Hz, 1H), 7.92 (d, J=8.0 Hz, 1H), 7.75-7.66 (m, 2H), 7.60-7.50 (m, 2H), 7.37 (t, J=7.6 Hz, 1H), 7.26-7.20 (m, 2H), 7.19-7.12 (m, 1H), 7.02 (d, J=2.0 Hz, 1H), 6.79 (dd, J=10.6, 16.8 Hz, 1H), 6.25 (dd, J=1.6, 16.8 Hz, 1H), 5.78 (dd, J=1.6, 10.6 Hz, 1H), 4.50 (d, J=14.0 Hz, 1H), 4.45 (d, J=7.2 Hz, 1H), 3.96 (dd, J=3.2, 11.4 Hz, 1H), 3.93-3.45 (m, 40H), 3.20 (s, 3H), 1.41 (d, J=6.8 Hz, 3H), 1.32 (d, J=6.8 Hz, 3H). LC-MS: MS (ES⁺): RT=1.977 min, m/z=1142.4 [M+H⁺], LC-MS METHOD 25.

### Example 14—Synthesis of Compound I-14 (Synthesized According to General Scheme C in a Similar Manner to Compound I-14)

3-[2,4-bis[(3R)-3-methylmorpholin-4-yl]pyrido[2,3-d]pyrimidin-7-yl]-N-[2-[2-[2-[2-[2-[2-[2-[2-[2-[[6-chloro-8-fluoro-7-(3-hydroxy-1-naphthyl)-4-(4-prop-2-enoylpiperazin-1-yl)quinazolin-2-yl]-methyl-amino]ethoxy]ethoxy]ethoxy]ethoxy]ethoxy]ethoxy]ethoxy]ethoxy]ethyl]benzamide (24 mg, 18 μmol, 34% yield, 100% purity) as a yellow solid. ¹H NMR (400 MHz, Methanol-d₄): δ8.62 (s, 1H), 8.37-8.13 (m, 2H), 7.97 (d, J=8.0 Hz, 1H), 7.82-7.70 (m, 2H), 7.67-7.56 (m, 2H), 7.40 (t, J=7.2 Hz, 1H), 7.30-7.23 (m, 2H), 7.23-7.16 (m, 1H), 7.04 (s, 1H) 6.84 (dd, J=16.8, 10.4 Hz, 1H), 6.28 (dd, J=16.8, 2.0 Hz, 1H), 5.81 (dd, J=10.4, 1.6 Hz, 1H), 4.63 (s, 2H), 4.58-4.46 (m, 2H), 4.04-3.87 (m, 9H), 3.87-3.78 (m, 6H), 3.77-3.59 (m, 8H), 3.66-3.47 (m, 32H), 3.27 (s, 3H), 1.46 (d, J=6.8 Hz, 3H), 1.36 (d, J=6.4 Hz, 3H). LC-MS: MS (ES⁺): RT=1.999 min, m/z=1320.0 [M+H⁺], LC-MS METHOD 25.

### Example 15—Synthesis of Compound I-15 (Synthesized According to General Scheme C in a Similar Manner to Compound I-12)

3-[2,4-bis[(3R)-3-methylmorpholin-4-yl]pyrido[2,3-d]pyrimidin-7-yl]-N-[2-[2-[2-[2-[2[2[2-[2-[2-[2-[2-[[6-chloro-8-fluoro-7-(3-hydroxy-1-naphthyl)-4-(4-prop-2-enoylpiperazin-1-yl)quinazolin-2-yl]-methyl-amino]ethoxy]ethoxy]ethoxy]ethoxy]ethoxy]ethoxy]ethoxy]ethoxy]ethoxy]ethoxy]ethyl]benzamide (43 mg, 31 μmol, 57% yield, 100% purity) as a yellow solid. LC-MS: MS (ES⁺): RT=2.754 min, m/z=1406.7 [M+H⁺]; LC-MS METHOD 01. ¹H NMR (400 MHz, MeOD): δ8.60 (s, 1H), 8.27 (d, J=7.6 Hz, 1H), 8.19 (d, J=8.4 Hz, 1H), 7.95 (d, J=7.0 Hz, 1H), 7.77-7.66 (m, 2H), 7.63-7.55 (m, 2H), 7.37 (t, J=7.0 Hz, 1H), 7.28-7.11 (m, 3H), 7.03 (s, 1H), 6.80 (dd, J=16.8, 10.8 Hz, 1H), 6.26 (dd, J=16.8, 2.0 Hz, 1H), 5.78 (dd, J=10.8, 2.0 Hz, 1H), 4.56-4.40 (m, 2H), 4.00-3.81 (m, 10H), 3.81-3.66 (m, 14H), 3.63-3.46 (m, 40H), 3.25 (s, 3H), 1.43 (d, J=6.8 Hz, 3H), 1.33 (d, J=6.8 Hz, 3H).

### Example 16—Synthesis of Compound I-16

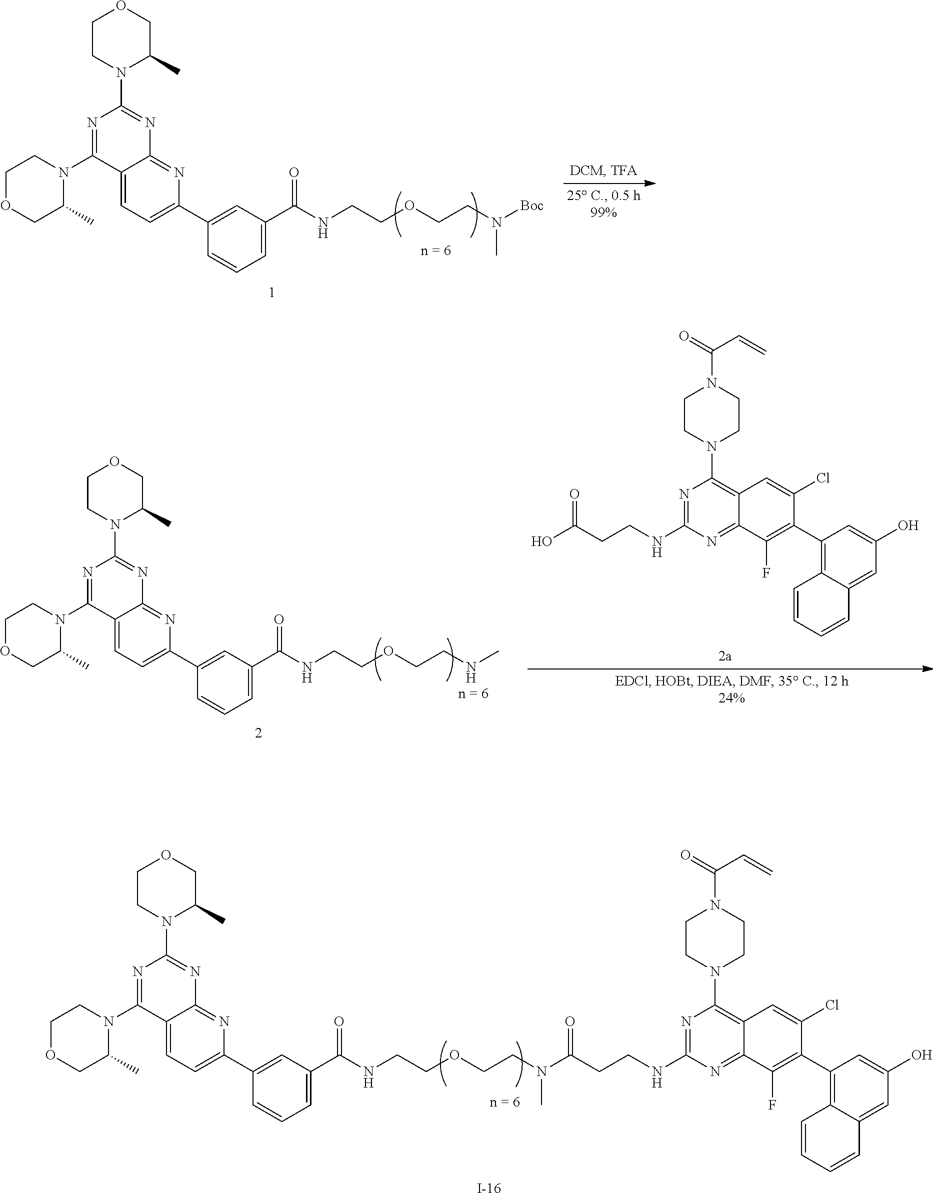

### 16.1 Preparation of Compound 2

To a solution of tert-butyl N-[2-[2-[2-[2-[2-[2-[2-[[3-[2,4-bis[(3R)-3-methylmorpholin-4-yl]pyrido[2,3-d]pyrimidin-7-yl]benzoyl]amino]ethoxy]ethoxy]ethoxy]ethoxy]ethoxy]ethoxy]ethyl]-N-methyl-carbamate (240 mg, 0.28 mmol, 1.0 equiv) in DCM (2 mL) was added TFA (1.02 g, 8.94 mmol, 0.6 mL, 32.4 equiv). The mixture was stirred at 25° C. for 0.5 h. The mixture was concentrated to give the desired product 3-[2,4-bis[(3R)-3-methylmorpholin-4-yl]pyrido[2,3-d]pyrimidin-7-yl]-N-[2-[2-[2-[2-[2-[2-[2-(methylamino)ethoxy]ethoxy]ethoxy]ethoxy]ethoxy]ethoxy]ethyl]benzamide (242 mg, 0.27 mmol, 99% yield, TFA salt) as a yellow oil. LC-MS: MS (ES⁺): RT=0.633 min, m/z=770.3 [M+H⁺].

### 16.2 Preparation of Compound I-16

To a solution of 3-[[6-chloro-8-fluoro-7-(3-hydroxy-1-naphthyl)-4-(4-prop-2-enoylpiperazin-1-yl)quinazolin-2-yl]amino]propanoic acid (40 mg, 73 μmol, 1.0 equiv) in DMF (1 mL) was added EDCI (28 mg, 0.10 mmol, 2.0 equiv), DIEA (47 mg, 0.4 mmol, 5.0 equiv), 3-[2,4-bis[(3R)-3-methylmorpholin-4-yl]pyrido[2,3-d]pyrimidin-7-yl]-N-[2-[2-[2-[2-[2-[2-[2-(methylamino)ethoxy]ethoxy]ethoxy]ethoxy]ethoxy]ethoxy]ethyl]benzamide (64 mg, 73 μmol, 1.0 equiv, TFA salt) and HOBt (20 mg, 0.1 mmol, 2.0 equiv). The mixture was stirred at 35° C. for 12 h. The mixture was concentrated to give a residue. The residue was purified by prep-HPLC (column: Unisil 3-100 C₁₈ Ultra 150*50 mm*3 um; mobile phase: [water (0.225% FA)-ACN]; B%: 25%-55%, 10 min) to give the desired product 3-[2,4-bis[(3R)-3-methylmorpholin-4-yl]pyrido[2,3-d]pyrimidin-7-yl]-N—[ [2[2-2-1[2-[2-[2-[13-[[6-chloro-8-fluoro-7-(3-hydroxy-1-naphthyl)-4-(4-prop-2-enoylpiperazin-1-yl)quinazolin-2-yl]amino]propanoyl-methylamino]ethoxy]ethoxy]ethoxy]ethoxy]ethoxy]ethoxy]ethyl]benzamide (22 mg, 17 μmol, 24% yield, 100% purity) as a yellow solid. ¹H NMR (400 MHz, DMSO-d₆): δ10.28-9.78 (m, 1H), 8.72 (t, J=5.4 Hz, 1H), 8.64 (s, 1H), 8.29 (d, J=7.8 Hz, 1H), 8.22 (d, J=8.4 Hz, 1H), 7.96 (d, J=7.6 Hz, 1H), 7.81-7.76 (m, 2H), 7.70 (d, J=8.4 Hz, 1H), 7.61 (t, J=7.8 Hz, 1H), 7.42 (td, J=4.0, 8.4 Hz, 1H), 7.26 (d, J=2.4 Hz, 1H), 7.21 (d, J=3.8 Hz, 2H), 7.04 (s, 1H), 6.84 (dd, J=10.4, 16.6 Hz, 1H), 6.17 (dd, J=2.4, 16.6 Hz, 1H), 5.85-5.64 (m, 1H), 4.76 (d, J=4.9 Hz, 1H), 4.43 (d, J=13.2 Hz, 2H), 3.98-3.87 (m, 3H), 3.84-3.68 (m, 10H), 3.67-3.60 (m, 4H), 3.58-3.49 (m, 10H), 3.49-3.38 (m, 24H), 2.99 (s, 1H), 2.80 (s, 1H), 2.67-2.58 (m, 2H), 1.37 (d, J=6.8 Hz, 3H), 1.24 (d, J=6.8 Hz, 3H). LC-MS: MS (ES⁺): RT=2.037 min, m/z=1302.6 [M+H⁺], LC-MS METHOD 25.

### Example 17—Synthesis of Compound I-17

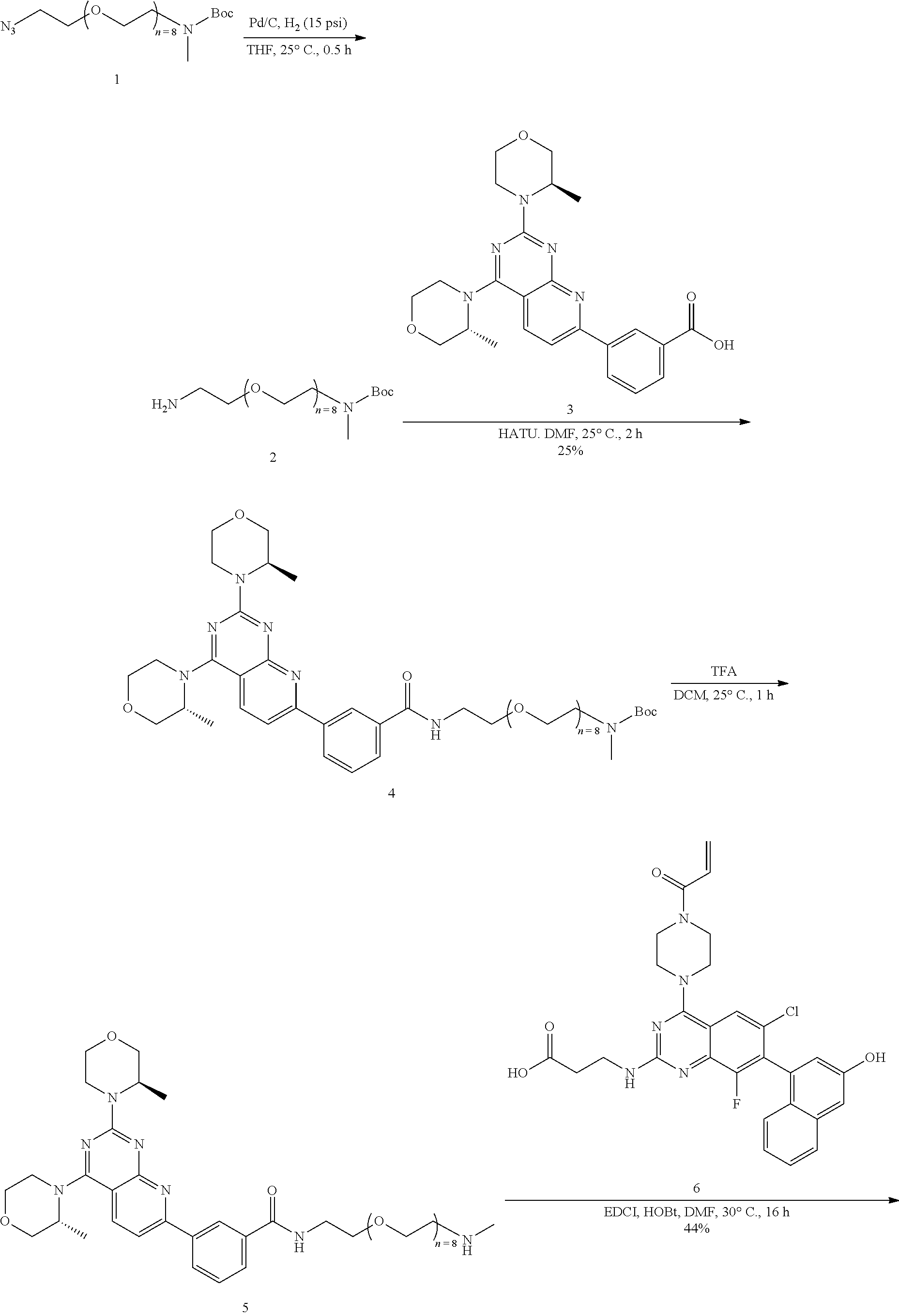

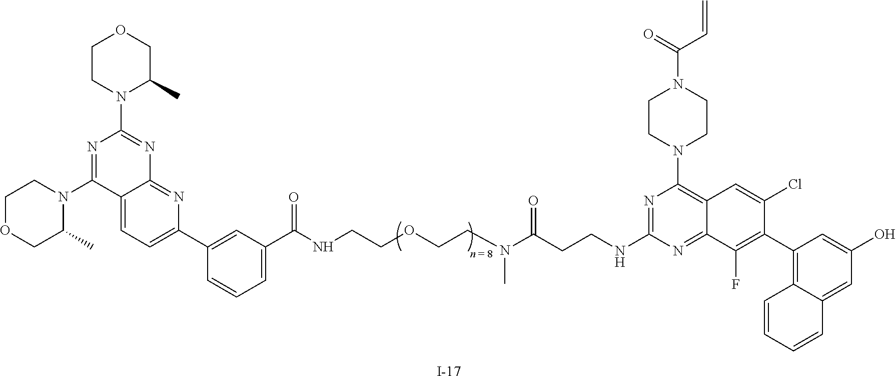

### 17.1 Preparation of Compound 2

To a solution of tert-butyl-N-[2-[2-[2-[2-[2-[2-[2-[2-(2-azidoethoxy)ethoxy]ethoxy]ethoxy]ethoxy]ethoxy]-ethoxy]ethoxy]ethyl]-N-methyl-carbamate (400 mg, 724 μmol, 1.0 equiv) in THF (4 mL) was added Pd/C (50 mg, 10% purity) under N₂. The suspension was degassed under vacuum and purged with H₂ for several times. The mixture was stirred under H₂ (15 psi, balloon) at 25° C. for 30 min. The mixture was filtered. The filtrate was concentrated to give crude tert-butylN-[2-[2-[2-[2-[2-[2-[2-[2-(2-aminoethoxy)ethoxy]ethoxy]ethoxy]ethoxy]ethoxy]ethoxy]-ethoxy]ethyl]-N-methyl-carbamate (350 mg, crude) as a colorless oil.

### 17.2 Preparation of Compound 4

To a solution of and 3-[2,4-bis[(3R)-3-methylmorpholin-4-yl]pyrido[2,3-d]pyrimidin-7-yl]benzoic acid (299 mg, 665 μmol, 1.0 equiv) and tert-butyl-N-[2-[2-[2-[2-[2-[2-[2-[2-(2-aminoethoxy)-ethoxy]ethoxy]ethoxy]ethoxy]ethoxy]ethoxy]ethoxy]ethyl]-N-methyl-carbamate (350 mg, 665 μmol, 1.0 equiv) in DMF (3 mL) were added DIEA (257 mg, 1.99 mmol, 3.0 equiv) and HATU (303 mg, 797 μmol, 1.2 equiv). The mixture was stirred at 25° C. for 2 h. The solution was purified by prep-HPLC (column: Phenomenex luna C18 150*40 mm* 15 um; mobile phase: [water(0.1% TFA)-ACN]; B%: 26%-56%, 11 min) to give tert-butyl-N-[2-[2-[2-[2-[2-[2-[2-[2-[2-[[3-[2,4-bis [(3R)-3-methylmorpholin-4-yl]pyrido[2,3-d]pyrimidin-7-yl]benzoyl]amino]ethoxy]ethoxy]ethoxy]ethoxy]ethoxy]ethoxy]ethoxy]ethoxy]ethyl]-N-methyl-carbamate (160 mg, 167 μmol, 25% yield) as a yellow gum. LC-MS: MS (ES⁺): RT=0.686 min, m/z=958.5 [M+H⁺].

### 17.3 Preparation of Compound 5

To a solution of tert-butyl-N-[2-2-[2-[2-[2-[2-[2-[2-[2-[-[[3-[2,4-bis[(3R)-3-methylmorpholin-4-yl]pyrido[2,3-d]pyrimidin-7-yl]benzoyl]amino]ethoxy]ethoxy]ethoxy]ethoxy]ethoxy]ethoxy]ethoxy]ethoxy]ethyl]-N-methyl-carbamate (83 mg, 86 μmol, 1.0 equiv) in DCM (1 mL) was added TFA (770 mg, 6.75 mmol, 0.5 mL, 78.0 equiv). The mixture was stirred at 25° C. for 1 h. The mixture was concentrated to give crude 3-[2,4-bis[(3R)-3-methylmorpholin-4-yl]pyrido[2,3-d]pyrimidin-7-yl]-N-[2-[2-[2-[2-[2-[2-[2-[2-[2-(methylamino)ethoxy]ethoxy]ethoxy]ethoxy]ethoxy]ethoxy]ethoxy]ethoxy]ethyl]benzamide (84 mg, crude, TFA salt) as a yellow gum. LC-MS: MS (ES⁺): RT=0.783 min, m/z=858.7 [M+H⁺].

### 17.4 Preparation of Compound I-17

To a solution of 3-[2,4-bis[(3R)-3-methylmorpholin-4-yl]pyrido[2,3-d]pyrimidin-7-yl]-N-[2-[2-[2-[2-[2-[2-[2-[2-[2-(methylamino)ethoxy]ethoxy]ethoxy]ethoxy]ethoxy]ethoxy]ethoxy]ethoxy]ethyl]benzamide (84 mg, 86 μmol, 1.0 equiv, TFA salt), EDCI (33 mg, 0.17 mmol, 2.0 equiv), HOBt (23 mg, 0.17 mmol, 2.0 equiv) and 3-[[6-chloro-8-fluoro-7-(3-hydroxy-1-naphthyl)-4-(4-prop-2-enoylpiperazin-1-yl)quinazolin-2-yl]amino]propanoic acid (47 mg, 86 μmol, 1.0 equiv) in DMF (1.5 mL) was added DIEA (56 mg, 0.43 mmol, 5.0 equiv). The mixture solution was stirred at 30° C. for 16 h. The solution was purified by prep-HPLC (column: Unisil 3-100 C18 Ultra 150*50 mm*3 um; mobile phase: [water(0.225% FA)-ACN]; B%: 18%-48%, 10 min) to give 3-[2,4-bis[(3R)-3-methylmorpholin-4-yl]pyrido[2,3-d]pyrimidin-7-yl]-N—[[2[2-[2-[2-[2-[2-[2-[2-2-[3-[[6-chloro-8-fluoro-7-(3-hydroxy-1-naphthyl)-4-(4-prop-2-enoylpiperazin-1-yl)quinazolin-2-yl]amino]propanoyl-methyl-amino]ethoxy]ethoxy]ethoxy]ethoxy]ethoxy]ethoxy]ethoxy]ethoxy]ethyl]benzamide (53 mg, 38 μmol, 44% yield, 100% purity) as a yellow solid. LC-MS: MS (ES⁺): RT=2.029 min, m/z=695.5 [1/2M+H⁺], LC-MS METHOD 25. ¹H NMR (400 MHz, MeOD): δ8.60 (s, 1H), 8.33-8.15 (m, 2H), 7.95 (d, J=8.0 Hz, 1H), 7.83-7.68 (m, 2H), 7.65-7.55 (m, 2H), 7.38 (t, J=7.6 Hz, 1H), 7.30-7.13 (m, 3H), 7.03 (s, 1H), 6.87-6.73 (m, 1H), 6.26 (d, J=16.8 Hz, 1H), 5.79 (d, J=10.4 Hz, 1H), 4.50 (d, J=13.6 Hz, 2H), 3.99-3.66 (m, 22H), 3.64-3.43 (m, 37H), 3.12-3.09 (m, 1H), 2.91-2.90 (m, 1H), 2.85-2.68 (m, 2H), 1.44 (d, J=6.4 Hz, 3H), 1.33 (d, J=6.4 Hz, 3H).

### Example 18—Synthesis of Compound I-18

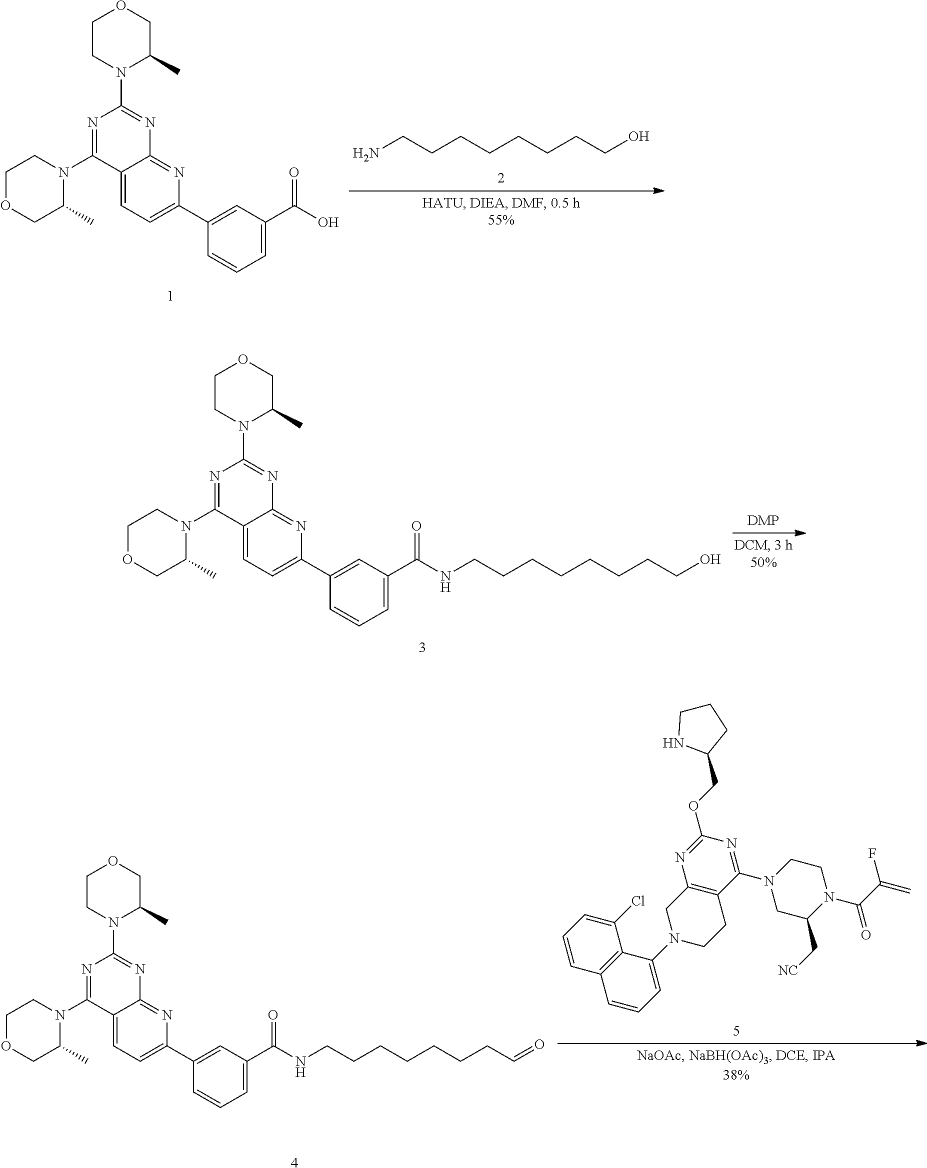

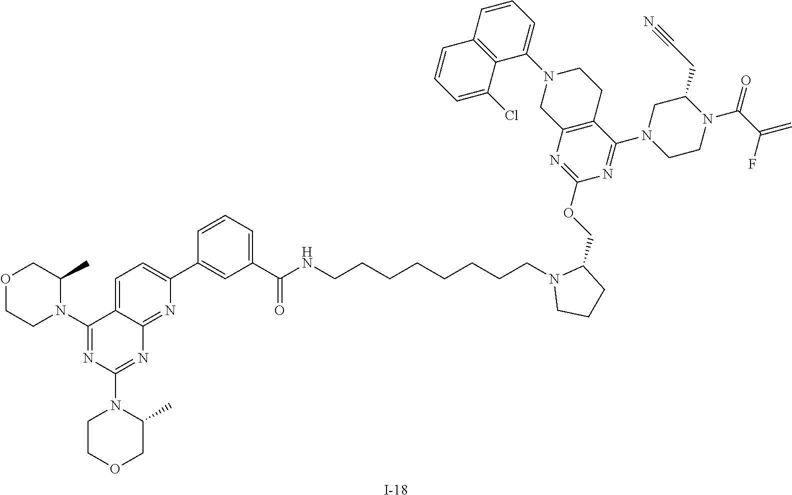

### 18.1 Preparation of Compound 3

To a solution of 8-aminooctan-1-ol (100 mg, 688 μmol, 1.0 equiv) and DIEA (267 mg, 2 mmol, 3.0 equiv) in DMF (4 mL) was added HATU (314 mg, 826 μmol, 1.2 equiv) and 3-[2,4-bis[(3R)-3-methylmorpholin-4-yl]pyrido[2,3-d]pyrimidin-7-yl]benzoic acid (309 mg, 688 μmol, 1.0 equiv). The solution was purified by prep-HPLC (column: Waters Xbridge C18 150*50 mm* 10 um; mobile phase: [water(l0 mM NH₄HCO₃)-ACN]; B%: 34%-64%,min) to give 3-[2,4-bis[(3R)-3-methylmorpholin-4-yl]pyrido[2,3-d]pyrimidin-7-yl]-N-(8-hydroxyoctyl)benzamide (220 mg, 55% yield) as a yellow solid. LC-MS: MS (ES⁺): m/z=577.4 [M+H⁺].

### 18.2 Preparation of Compound 4

To a mixture of 3-[2,4-bis[(3R)-3-methylmorpholin-4-yl]pyrido[2,3-d]pyrimidin-7-yl]-N-(8-hydroxyoctyl)benzamide (100 mg, 173 μmol, 1.0 equiv) in DCM (3 mL) was added Dess-Martin (110 mg, 260 μmol, 1.5 equiv). The mixture was stirred at 20° C. for 3 h and purified by prep-TLC (DCM/MeOH=10/1) to give 3-[2,4-bis[(3R)-3-methylmorpholin-4-yl]pyrido[2,3-d]pyrimidin-7-yl]-N-(8-oxooctyl)benzamide (50 mg, 50% yield) as a yellow gum. LC-MS: MS (ES⁺): m/z=575.3 [M+H⁺].

### 18.3 Preparation of Compound I-18

To a mixture of 3-[2,4-bis[(3R)-3-methylmorpholin-4-yl]pyrido[2,3-d]pyrimidin-7-yl]-N-(8-oxooctyl) benzamide (50 mg, 87 μmol, 1.0 equiv), NaOAc (21 mg, 261 μmol, 3.0 equiv) and NaBH(OAc)₃ (92 mg, 435 μmol, 5.0 equiv) in DCE (5 mL) and IPA (5 mL) was added 2-[(2S)-4-[7-(8-chloro-1-naphthyl)-2-[[(2S)-pyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (61 mg, 87 mol, 1.0 equiv, TFA salt). The mixture was stirred at 20° C. for 12 h and purified by prep-HPLC (column: Phenomenex luna C18 150*25 mm* 10 um; mobile phase: [water(0.225% FA)-ACN]; B%: 17%-47%, 10 min) to give 3-[2,4-bis[(3R)-3-methylmorpholin-4-yl]pyrido[2,3-d]pyrimidin-7-yl]-N-[8-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido [3,4-d]pyrimidin-2-yl]oxymethyl]pyrrolidin-1-yl]octyl]benzamide (45 mg, 38% yield) as a yellow solid. ¹H NMR (400 MHz, DMSO-d₆): δ8.68-8.58 (m, 2H), 8.30 (d, 1H, J=8.0 Hz), 8.24 (d, 1H, J=8.4 Hz), 7.99-7.94 (m, 2H), 7.77-7.67 (m, 2H), 7.63-7.49 (m, 3H), 7.47-7.41 (m, 1H), 7.38-7.31 (m, 1H), 5.42-5.19 (m, 2H), 4.78 (s, 2H), 4.44 (d, 2H, J=13.4 Hz), 4.29-4.13 (m, 2H), 4.05-3.89 (m, 6H), 3.78-3.56 (m, 8H), 3.48-3.41 (m, 1H), 3.30-3.22 (m, 4H), 3.15-2.91 (m, 6H), 2.83-2.65 (m, 3H), 2.28 (s, 1H), 2.16 (d, 1H, J=7.2 Hz), 1.91-1.81 (m, 1H), 1.70-1.57 (m, 3H), 1.56-1.33 (m, 8H), 1.29-1.22 (m, 12H). LC-MS: MS (ES⁺): RT=2.186 min, m/z=574.8 [M/2+H⁺], LC-MS METHOD 25.

### Example 19—Synthesis of Compound I-19 (synthesized according to General Scheme D)

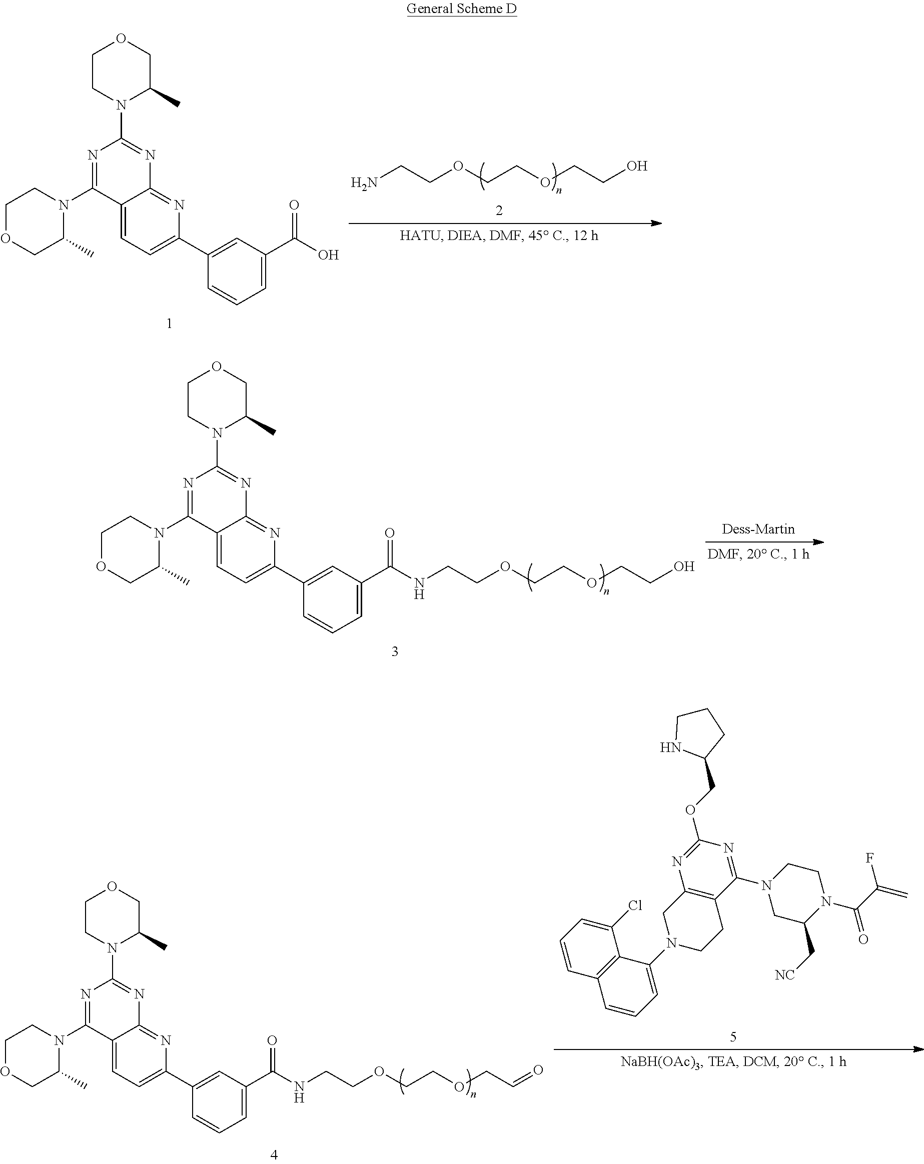

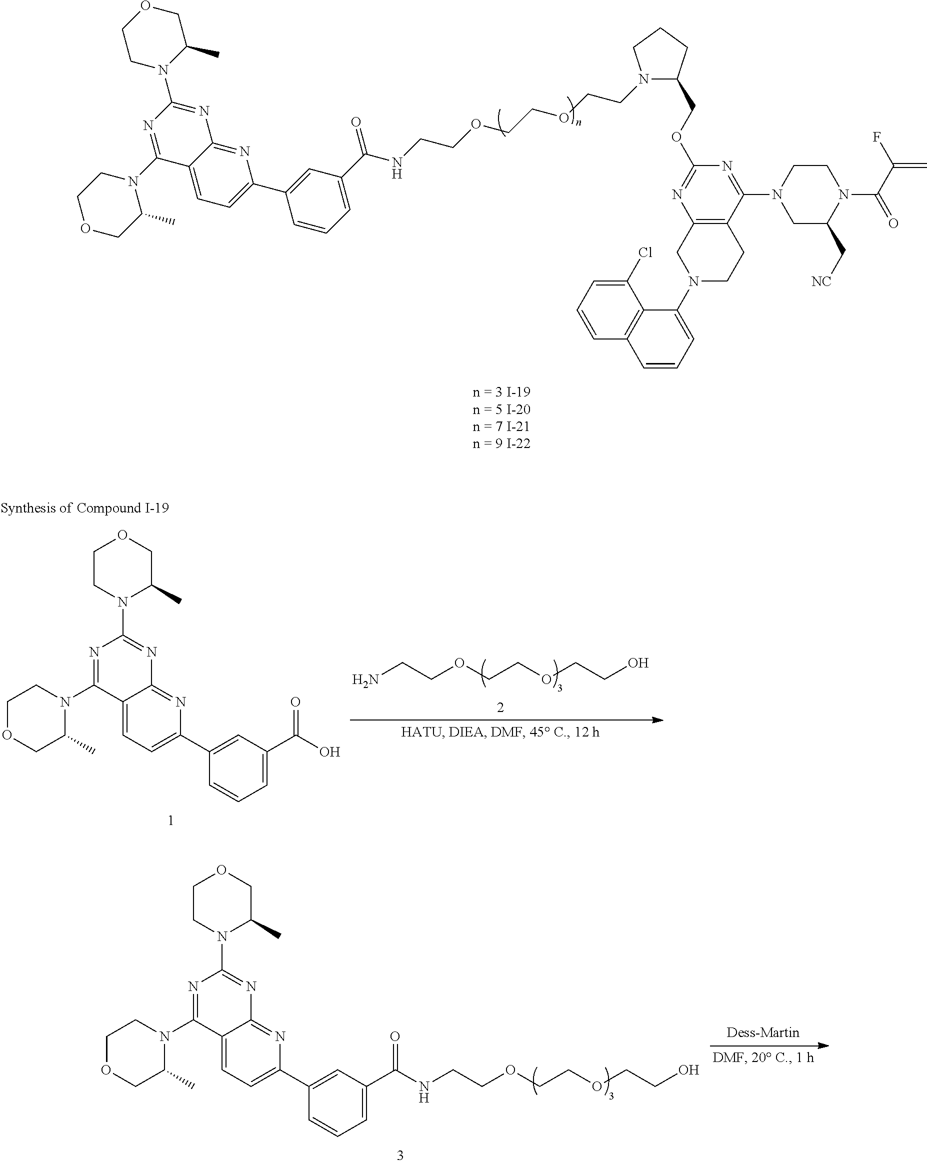

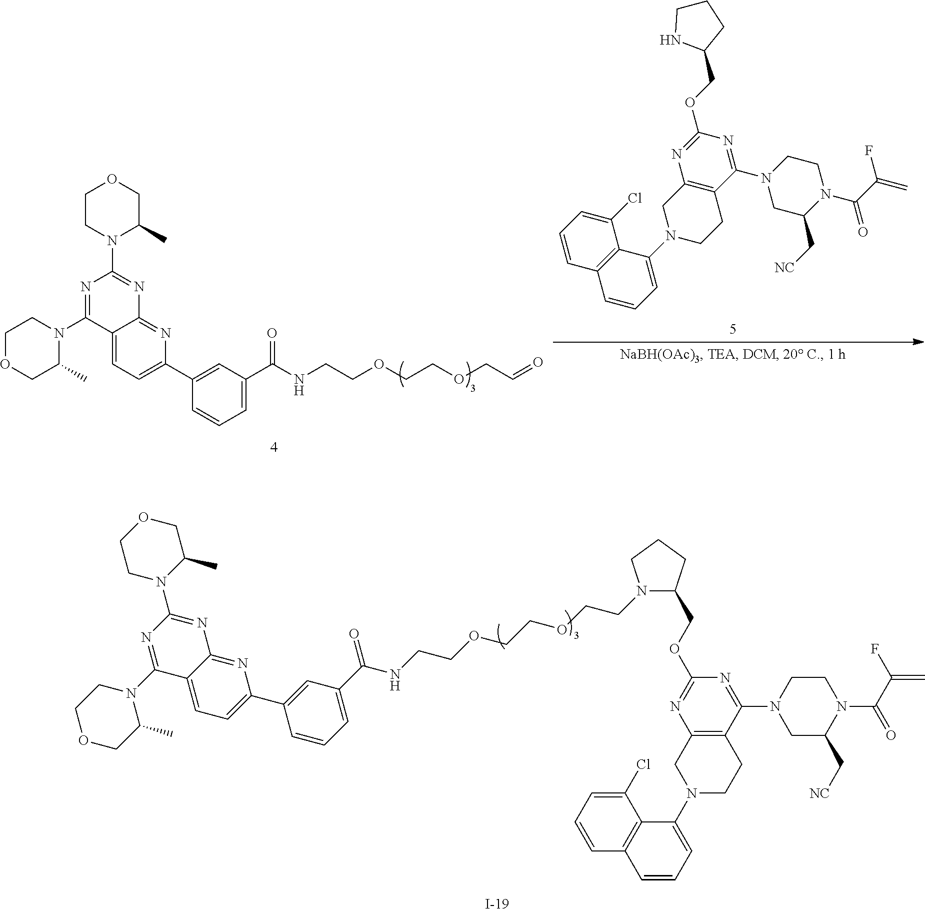

### 19.1 Preparation of Compound 3

To a solution of 3-[2,4-bis[(3R)-3-methylmorpholin-4-yl]pyrido[2,3-d]pyrimidin-7-yl]benzoic acid (200 mg, 444 μmol, 1 equiv) and 2-[2-[2-[2-(2-aminoethoxy)ethoxy]ethoxy]ethoxy]ethanol (156 mg, 444 μmol, 1 equiv) in DMF (1 mL) was added DIEA (181 mg, 1.41 mmol, 3.2 equiv) and HATU (203 mg, 533 μmol, 1.2 equiv). The mixture was stirred at 45° C. for 12 h and purified by prep-HPLC (column: Phenomenex Synergi C18 150*25 mm* 10 um; mobile phase: [water(0.1% TFA)-ACN]; B%: 22%-52%, 9 min) to give 3-[2,4-bis[(3R)-3-methylmorpholin-4-yl]pyrido [2,3-d]pyrimidin-7-yl]-N-[2-[2-[2-[2-(2-hydroxyethoxy)ethoxy]ethoxy]ethoxy]ethyl]benzamide (120 mg, 40% yield) as a yellow gum. ¹H NMR (400 MHz, DMSO-d₆) δ8.76-8.66 (m, 2H), 8.48-8.31 (m, 2H), 8.11-7.95 (m, 2H), 7.78-7.61 (m, 1H), 4.01-3.92 (m, 3H), 3.81-3.69 (m, 5H), 3.68-3.51 (m, 20H), 3.41-3.37 (m, 7H), 1.29-1.22 (m, 6H). LC-MS: MS (ES⁺): m/z=669.3 [M+H⁺].

### 19.2 Preparation of Compound 4

To a solution of 3-[2,4-bis[(3R)-3-methylmorpholin-4-yl]pyrido[2,3-d]pyrimidin-7-yl]-N-[2-[2-[2-[2-(2-hydroxyethoxy)ethoxy]ethoxy]ethoxy]ethyl]benzamide (61 mg, 91 μmol, 1 equiv) in DMF (2 mL) was added Dess-Martin (96 mg, 228 μmol, 2.5 equiv). The mixture was used directly for next step without further purification. LC-MS: MS (ES⁺): m/z=667.2 [M+H⁺].

### 19.3 Preparation of Compound I-19

To a mixture of 3-[2,4-bis[(3R)-3-methylmorpholin-4-yl]pyrido[2,3-d]pyrimidin-7-yl]-N-[2-[2-[2-[2-(2-oxoethoxy)ethoxy]ethoxy]ethoxy]ethyl]benzamide (60 mg, 89 μmol, 1 equiv), TEA (91 mg, 899 μmol, 10 equiv) and NaBH(OAc)₃ (190 mg, 899 μmol, 10 equiv) in DCM (1 mL) was added 2-[(2S)-4-[7-(8-chloro-1-naphthyl)-2-[[(2S)-pyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (63 mg, 89 μmol, 1.0 equiv, TFA salt). The mixture was stirred at 20° C. for 1 h and purified by prep-HPLC (column: Phenomenex luna C18 150*25 mm* 10 um; mobile phase: [water(0.225% FA)-ACN]; B%: 21%-51%, 10 min) to give 3-[2,4-bis[(3R)-3-methylmorpholin-4-yl]pyrido [2,3-d]pyrimidin-7-yl]-N-[2-[2-[2-[2-[2-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]ethoxy]ethoxy]ethoxy]ethoxy]ethyl]benzamide (6.08 mg, 5% yield) as a yellow solid. ¹H NMR (400 MHz, CD₃OD) δ8.68-8.49 (m, 2H), 8.35-8.18 (m, 2H), 8.02-7.90 (m, 1H), 7.85-7.76 (m, 1H), 7.68-7.56 (m, 3H), 7.50-7.23 (m, 3H), 5.42-5.21 (m, 2H), 4.55-4.08 (m, 1H), 4.04-3.81 (m, 1H), 3.70-3.50 (m, 28H), 3.46-3.32 (m, 4H), 3.23-2.78 (m, 10H), 2.73-2.49 (m, 2H), 2.19-2.03 (m, 1H), 1.80-1.72 (m, 3H), 1.47-1.43 (m, 3H), 1.35-1.26(m, 3H). LC-MS: MS (ES⁺): RT=2.20 min, m/z=620.5 [M/2+H⁺], LC-MS METHOD 25.

### Example 20—Synthesis of Compound I-20 (Synthesized According to General Scheme D in a Similar Manner to Compound I-19)

3-[2,4-bis[(3R)-3-methylmorpholin-4-yl]pyrido[2,3-d]pyrimidin-7-yl]-N-[2-[2-[2-[2-[2-[2-[2-1[(2S)-2-[[7-(8-chloro-1-naphthyl)-4-1[(3S)-3-(cyanomethyl)-4-(2-fluoroprop-2-enoyl)piperazin-1-yl]-6,8-dihydro-SH-pyrido[3,4-d]pyrimidin-2-yl]oxymethyl]pyrrolidin-1-yl]ethoxy]ethoxy]ethoxy]ethoxy]ethoxy]ethoxy]ethyl]benzamide (9 mg, 5% yield) as a yellow solid. ¹H NMR (400 MHz, CD₃OD): δ8.65 (s, 1H), 8.36-8.23 (m, 2H), 7.97 (d, J=8.0 Hz, 1H), 7.83 (d, J=8.0 Hz, 1H), 7.72-7.60 (m, 3H), 7.55-7.46 (m, 2H), 7.40-7.26 (m, 2H), 5.45-5.23 (m, 2H), 4.68-4.46 (m, 5H), 4.41-4.14 (m, 4H), 3.86-3.49 (m, 46H), 3.26-3.09 (m, 6H), 2.75-2.62 (m, 1H), 2.31 (s, 4H), 1.48 (d, J=6.4 Hz, 3H), 1.36 (d, J=6.8 Hz, 3H). LC-MS: MS (ES⁺): RT=2.074 min, m/z=1328.4 [M+H⁺], LC-MS METHOD 25.

### Example 21—Synthesis of Compound 1-21 (synthesized according to General Scheme D in a similar manner to Compound I-19)

3-[2,4-bis[(3R)-3-methylmorpholin-4-yl]pyrido[2,3-d]pyrimidin-7-yl]-N-[2-[2-[2-[2-[2-[2-[2-[2-[2-[(2S)-2-[[7-(8-chloro-1-naphthyl)-4-[(3S)-3-(cyanomethyl)-4-(2-fluoroprop-2-enoyl)piperazin-1-yl]-6,8-dihydro-SH-pyrido[3,4-d]pyrimidin-2-yl]oxymethyl]pyrrolidin-1-yl]ethoxy]ethoxy]ethoxy]ethoxy]ethoxy]ethoxy]ethoxy]ethoxy]ethyl]benzamide (12 mg, 12% yield) as a yellow solid. ¹H NMR (400 MHz, CD₃OD) δ8.70-8.47 (m, 2H), 8.35-8.21 (m, 2H), 8.01-7.92 (m, 1H), 7.85-7.77 (m, 1H), 7.68-7.61 (m, 2H), 7.55-7.43 (m, 2H), 7.40-7.23 (m, 2H), 5.52-5.18 (m, 2H), 4.61-4.25 (m, 6H), 4.22-4.09 (m, 2H), 4.06-3.89 (m, 4H), 3.89-3.44 (m, 43H), 3.43-3.32 (m, 6H), 3.27-3.09 (m, 4H), 2.99-2.83 (m, 2H), 2.78-2.56 (m, 2H), 2.20-2.03 (m, 1H), 1.99-1.72 (m, 3H), 1.51-1.40 (m, 3H), 1.38-1.30 (m, 3H). LC-MS: MS (ES⁺): RT=2.879 min, m/z=708.9 [M/2+H⁺], LC-MS METHOD 01.

### Example 22—Synthesis of Compound I-22 (synthesized according to General Scheme D in a similar manner to Compound I-19)

3-[2,4-bis[(3R)-3-methylmorpholin-4-yl]pyrido[2,3-d]pyrimidin-7-yl]-N—[2-[2-[2-[2-[2-[2-[2-[2-[2-2-[2-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]ethoxy]ethoxy]ethoxy]ethoxy]ethoxy]ethoxy]ethoxy]ethoxy]ethoxy]ethoxy]ethyl]benzamide (22.33 mg, 23% yield) as a yellow solid. ¹H NMR (400 MHz, CD₃OD): δ8.65 (s, 1H), 8.37-8.21 (m, 2H), 7.98 (d, J=7.6 Hz, 1H), 7.83 (d, J=8.0 Hz, 1H), 7.71-7.60 (m, 3H), 7.55-7.46 (m, 2H), 7.40-7.27 (m, 2H), 5.47-5.24 (m, 2H), 4.62-4.15 (m, 8H), 4.14-3.94 (m, 4H), 3.89-3.48 (m, 58H), 3.28-2.97 (m, 8H), 2.76-2.63 (m, 1H), 2.35-2.20 (m, 1H), 2.13-1.95 (m, 3H), 1.50-1.33 (m, 6H). LC-MS: MS (ES⁺): RT=2.816 min, m/z=752.9 [M/2+H⁺], LC-MS METHOD 01.

### Example 23—Synthesis of Compound I-23 (Synthesized according to General Scheme E)

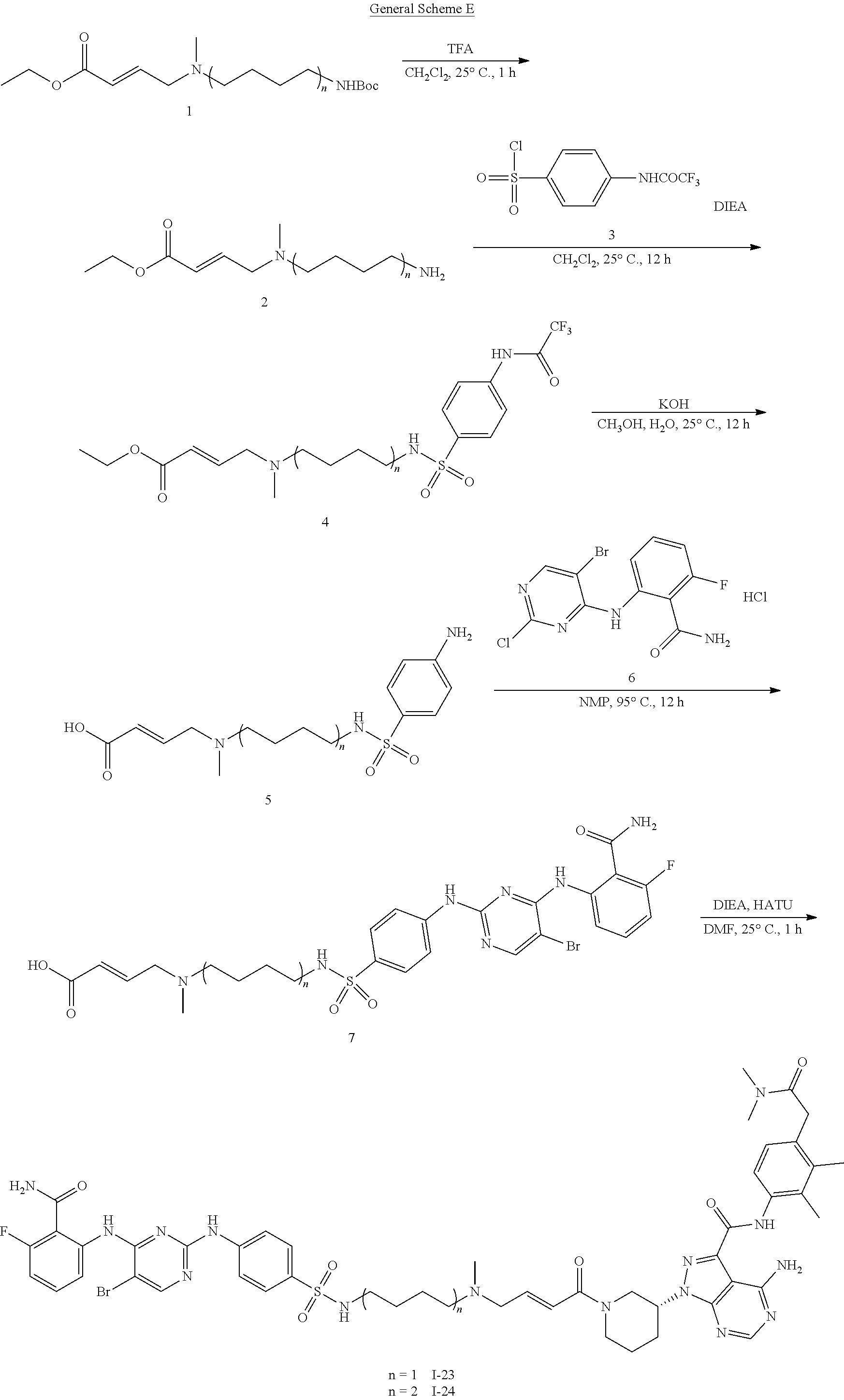

### 23.1 Preparation of Compound 2

To a solution of ethyl (E)-4-[4-(tert-butoxycarbonylamino)butyl-methyl-amino]but-2-enoate (500 mg, 1.59 mmol, 1.0 equiv) in CH₂Cl₂ (2 mL) was added Et₃N (1 mL), and it was stirred at 25° C. for 1 h. The mixture was concentrated to give the crude product ethyl (E)-4-[4-aminobutyl(methyl)amino]but-2-enoate (515 mg, crude, TFA salt) as a yellow oil.

### 23.2 Preparation of Compound 4

To a solution of ethyl €-4-[4-aminobutyl(methyl)amino]but-2-enoate (515 mg, 1.57 mmol, 1.0 equiv, TFA salt) and 4-[(2,2,2-trifluoroacetyl)amino]benzenesulfonyl chloride (677 mg, 2.35 mmol, 1.5 equiv) in CH₂Cl₂ (5 mL) was added DIEA (608 mg, 4.71 mmol, 820 μL, 3.0 equiv), and it was stirred at 25° C. for 2 h. To the reaction mixture was added water (50 mL) and the mixture was extracted with CH₂Cl₂ (50 mL). The combined organic phase was washed with brine (50 mL), dried over anhydrous Na₂SO₄, filtered and concentrated in vacuum. Then it was purified by prep-HPLC (column: Phenomenex luna C18 150*40 mm* 15 um; mobile phase: [water (0.1% TFA)-€]; B%: 14%-44%,llmin) to give ethyl €-4-[methyl-[4-[[4-[(2,2,2-trifluoroacetyl)amino]phenyl]sulfonylamino]butyl]amino]but-2-enoate (304 mg, 525 μmol, 33.4% yield, TFA salt) as a yellow gum. ¹H NMR (400 MHz, CD₃OD-d₄) δ 7.94-7.79 (m, 4H), 6.95-6.80 (m, 1H), 6.33 (d, J=15.6 Hz, 1H), 4.30-4.20 (m, 2H), 4.10-3.86 (m, 2H), 3.25-3.06 (m, 2H), 3.00-2.78 (m, 5H), 1.90-1.70 (m, 2H), 1.65-1.50 (m, 2H), 1.30 (t, J=7.2 Hz, 3H). LC-MS: MS (ES′): m/z=466.3 [M+H⁺].

### 23.3 Preparation of Compound 5

To a solution of ethyl €-4-[methyl-[4-[[4-[(2,2,2-trifluoroacetyl)amino]phenyl]sulfonylamino]butyl]amino]but-2-enoate (304 mg, 653 μmol, 1.0 equiv) in H₂O (2 mL) and CH₃OH (4 mL) was added KOH (183 mg, 3.27 mmol, 5.0 equiv), and it was stirred at 25° C. for 12 h. The pH was adjusted to 6-7 by 1 N HCl at 0° C., and then it was freeze-dried to afford crude product. THF (20 ml) was added and stirred for 0.5 h. After filtration, the filtrate was concentrated to afford crude product (E)-4-[4-[(4-aminophenyl)sulfonylamino]butyl-methyl-amino]but-2-enoic acid (220 mg, 644 μmol, crude) as a yellow gum. LC-MS: MS (ES⁺): m/z=342.2 [M+H⁺].

### 23.4 Preparation of Compound 7

To a solution of €-4-[4-[(4-aminophenyl)sulfonylamino]butyl-methyl-amino]but-2-enoic acid (220 mg, 644 μmol, 1.0 equiv) and 2-[(5-bromo-2-chloro-pyrimidin-4-yl)amino]-6-fluoro-benzamide (223 mg, 644 μmol, 1.0 equiv) in NMP (4 mL) was added HCl (64.0 mg, 644 mol, 62.0 μL, 37% purity, 1.0 equiv), and it was stirred at 95° C. for 12 h. H₂O (0.1 mL) was added to quench this reaction. It was purified by prep-HPLC (column: Phenomenex luna C18 150*40 mm* 15 um; mobile phase: [water (0.1% TFA)]; B%: 15%-45%, 11 min) to afford €-4-[4-[[4-[[5-bromo-4-(2-carbamoyl-3-fluoro-anilino)pyrimidin-2-yl]amino]phenyl]sulfonylamino]butyl-methyl-amino]but-2-enoic acid (80.0 mg, 105 μmol, 16.2% yield, TFA salt) as a yellow solid. LC-MS: MS (ES⁺): m/z=650.1 [M+H⁺].

### 23.5 Preparation of Compound I-23

To a solution of (E)-4-[4-[[4-[[5-bromo-4-(2-carbamoyl-3-fluoro-anilino)pyrimidin-2-yl]amino]phenyl]sulfonylamino]butyl-methyl-amino]but-2-enoic acid (80.0 mg, 104.6 μmol, 1.0 equiv, TFA salt), 4-amino-N-[4-[2-(dimethylamino)-2-oxo-ethyl]-2,3-dimethyl-phenyl]-1-[(3R)-3-piperidyl]pyrazolo[3,4-d]pyrimidine-3-carboxamide (59.0 mg, 105 μmol, 1.0 equiv, TFA salt) and DIEA (40.0 mg, 314 μmol, 54.7 μL, 3.0 equiv) in DMF (2 mL) was added HATU (47.0 mg, 126 μmol, 1.2 equiv), and it was stirred at 25° C. for 1 h. H₂O (0.1 mL) was added to quench this reaction. It was purified by prep-HPLC (column: Phenomenex luna C18 150*25 mm* 10 um; mobile phase: [water (0.05% HCl)-ACN]; B%: 17%-47%, 10 min) to afford 4-amino-1-[(3R)-1-[(E)-4-[4-[[4-[[5-bromo-4-(2-carbamoyl-3-fluoro-anilino)pyrimidin-2-yl]amino]phenyl]sulfonyl-amino]butyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (63 mg, 52 μmol, 49% yield, 92% purity, HCl salt) as a yellow solid. ¹H NMR (400 MHz, CD₃OD-d₄) δ 8.52-8.41 (m, 1H), 8.34 (d, J=8 Hz, 1H), 8.00-7.88 (m, 1H), 7.85-7.46 (m, 5H), 7.36-6.98 (m, 4H), 6.85-6.65 (m, 1H), 5.15-4.97 (m, 1H), 4.70-4.38 (m, 1H), 4.16-3.89 (m, 3H), 3.86-3.67 (m, 3H), 3.58-3.41 (m, 1H), 3.22-3.06 (m, 5H), 3.01 (s, 3H), 2.95-2.79 (m, 5H), 2.54-2.18 (m, 8H), 2.29-2.18 (m, 1H), 2.16-2.02 (m, 1H), 1.92-1.73 (m, 3H), 1.64-1.47 (m, 2H). LC-MS: MS (ES′): RT=2.270 min, m/z=1082.2 [M+H⁺]; LC-MS METHOD 10.

### Example 24—Synthesis of Compound I-24 (synthesized according to General Scheme E in a similar manner to Compound I-23)

4-amino-1-[(3R)-1-[(E)-4-[8-[[4-[[5-bromo-4-(2-carbamoyl-3-fluoro-anilino)pyrimidin-2-yl]amino]phenyl]sulfonylamino]octyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (22 mg, 19 μmol, 9.1% yield) as a white solid. ¹H NMR (400 MHz, CD₃OD-d₄) δ 8.53-8.39 (m, 1H), 8.35-8.25 (m, 1H), 8.11-7.97 (m, 1H), 7.79-7.71 (m, 2H), 7.70-7.62 (m, 2H), 7.56-7.44 (m, 1H), 7.37-7.23 (m, 1H), 7.18-6.95 (m, 3H), 6.76-6.61 (m, 1H), 5.16-4.97 (m, 1H), 4.73-4.28 (m, 1H), 4.19-3.67 (m, 6H), 3.64-3.38 (m, 1H), 3.22-3.05 (m, 5H), 3.03-2.94 (m, 3H), 2.92-2.78 (m, 5H), 2.53-2.30 (m, 2H), 2.29-2.17 (m, 6H), 2.16-2.01 (m, 1H), 1.89-1.61 (m, 3H), 1.55-1.41 (m, 2H), 1.40-1.22 (m, 8H). LC-MS: MS (ES′): RT=2.022 min, m/z=1139.3 [M+H⁺]; LC-MS METHOD 25.

### Example 25—Synthesis of Compound I-25 (Synthesized from 8a below according to General Scheme F)

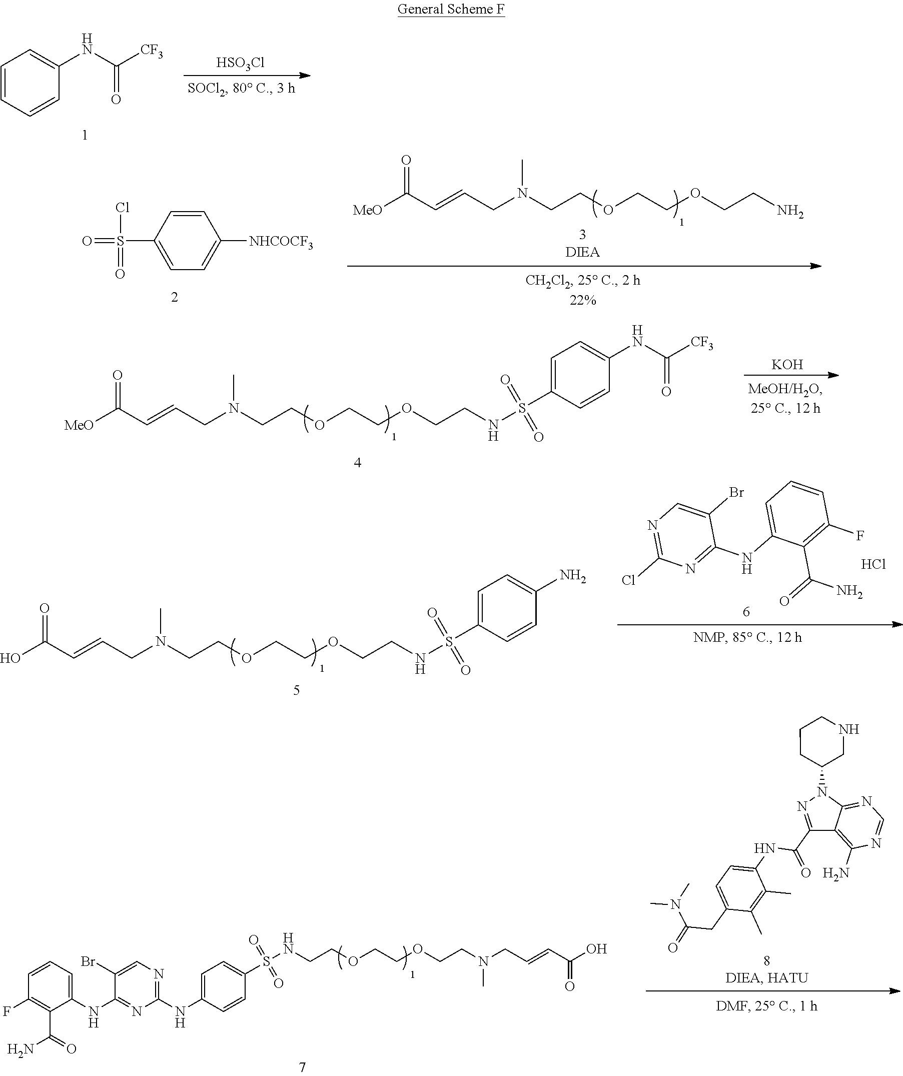

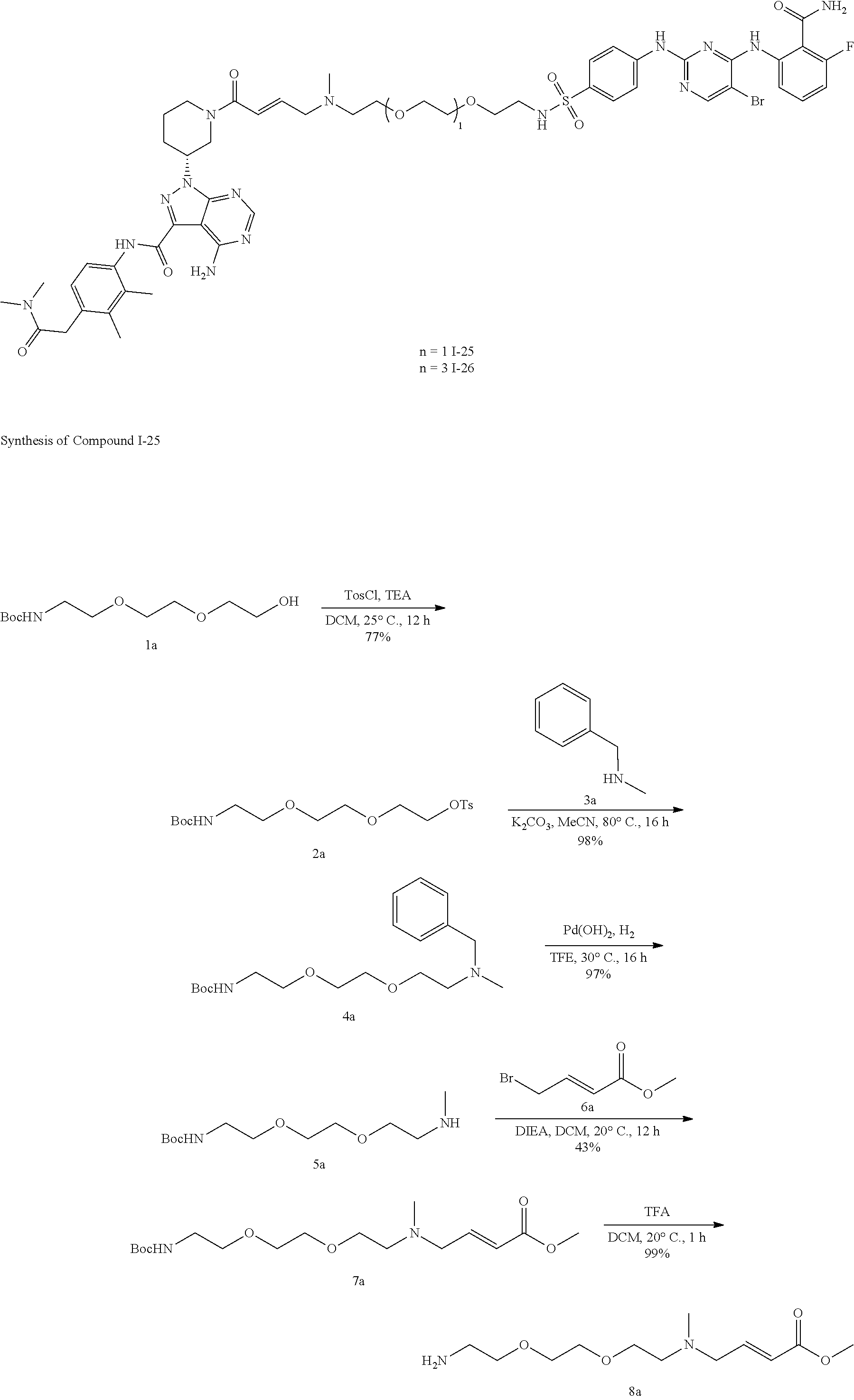

### 25.1 Preparation of Compound 2a

To a solution of compound 1a (2.00 g, 8.02 mmol, 1.0 equiv) in DCM (20 mL) was added TosCl (2.29 g, 12.03 mmol, 1.5 equiv) and TEA (2.44 g, 24.07 mmol, 3.35 mL, 3.0 equiv). The mixture was stirred at 25° C. for 12 h. The reaction mixture was concentrated under reduced pressure to give a residue. The residue was purified by column chromatography (SiO₂, Petroleum ether/Ethyl acetate=10/1 to 2/1). Compound 2a (2.50 g, 6.20 mmol, 77% yield) was obtained as a colorless oil. ¹HNMR (400 MHz, CDCl₃): δ7.81 (d, J=8.2 Hz, 2H), 7.35 (d, J=8.1 Hz, 2H), 5.09-4.82 (m, 1H), 4.25-4.12 (m, 2H), 3.76-3.67 (m, 2H), 3.61-3.53 (m, 4H), 3.53-3.47 (m, 2H), 3.30 (s, 2H), 2.46 (s, 3H), 1.44 (s, 9H). LC-MS: MS (ES⁺): m/z=426.2 [M+Na⁺].

### 25.2 Preparation of Compound 4a

To a solution of compound 2a (2.50 g, 6.20 mmol, 1.0 equiv) and compound 3a (750 mg, 6.20 mmol, 799 μL, 1.0 equiv) in MeCN (20 mL) was added K₂CO₃ (1.71 g, 12.4 mmol, 2.0 equiv). The mixture was stirred at 80° C. for 16 h. The mixture was filtered and concentrated to give a residue. The residue was purified by column chromatography (SiO₂, Petroleum ether/Ethyl acetate=2/1 to 0/1). Compound 4a (2.15 g, 6.10 mmol, 98% yield) was obtained as a colorless oil. ¹HNMR (400 MHz, CDCl₃): δ7.27 (m, 4H), 7.21-7.16 (m, 1H), 5.12-4.93 (m, 1H), 3.61-3.42 (m, 10H), 3.34-3.17 (m, 2H), 2.65-2.53 (m, 2H), 2.23 (s, 3H), 1.39 (s, 9H). LC-MS: MS (ES⁺): m/z=353.3 [M+H⁺].

### 25.3 Preparation of Compound 5a

To a solution of compound 4a (2.15 g, 6.10 mmol, 1.0 equiv) in TFE (30 mL) was added Pd(OH)₂ (200 mg, 10% purity) under N₂ atmosphere. The suspension was degassed and purged with H₂ for 3 times. The mixture was stirred under H₂ (50 Psi) at 30° C. for 16 h. The reaction mixture was filtered and concentrated under reduced pressure to give crude compound 5a (1.57 g, 5.97 mmol, 97% yield) as a colorless oil. The crude product was used directly for next step. LC-MS: MS (ES⁺): m/z=263.0 [M+H⁺].

### 25.4 Preparation of Compound 7a

To a solution of compound 5a (500 mg, 1.91 mmol, 1.0 equiv) and DIEA (493 mg, 3.82 mmol, 665 μL, 2.0 equiv) in DCM (8 mL) was added compound 6a (410 mg, 2.29 mmol, 269 μL, 1.2 equiv). The mixture was stirred at 20° C. for 12 h. The reaction mixture was concentrated under reduced pressure. The residue was purified by column chromatography (SiO₂, Petroleum ether/Ethyl acetate=1/1 to Ethyl acetate/Methanol=10/1). Compound 7a (300 mg, 832 μmol, 43% yield) was obtained as a yellow oil. ¹HNMR (400 MHz, CDCl₃): δ7.05-6.91 (m, 1H), 6.09-5.91 (d, J=16.0 Hz, 1H), 3.75 (s, 3H), 3.66-3.49 (m, 8H), 3.37-3.28 (m, 2H), 3.26-3.19 (m, 2H), 2.67-2.58 (m, 2H), 2.31 (s, 3H), 1.45 (s, 9H). LC-MS: MS (ES⁺): m/z=361.2 [M+H⁺].

### 25.5 Preparation of Compound 8a

To a solution of compound 7a (300 mg, 832 μmol, 1.0 equiv) in DCM (4 mL) was added TFA (1 mL). The mixture was stirred at 20° C. for 1 h. The reaction mixture was concentrated under reduced pressure to give crude compound 8a (311 mg, 830 μmol, crude, TFA salt). The crude product was used directly for next step. LC-MS: MS (ES⁺): m/z=260.9 [M+H⁺].

### 25.6 Preparation of Compound 2 (General Scheme F)

To a solution of 2,2,2-trifluoro-N-phenyl-acetamide (5.00 g, 26.4 mmol, 1.0 equiv) in SOCl₂ (30 mL) was added chlorosulfonic acid (9.24 g, 79.3 mmol, 5.30 mL, 3.0 equiv), and then it was stirred at 80° C. for 3 h. The reaction mixture was concentrated and 20 mL CH₂Cl₂ was added. The resulting solution was slowly added to 500 mL sat.NaHCO₃ at 0° C. and stirred for 30 min. After extracted with EtOAc (3×80 mL), the organic layers were dried over anhydrous Na₂SO₄, filtered and concentrated to afford crude product. The residue was purified by silica chromatography (Petroleum ether/Ethyl acetate=5:1-3:1) to afford 4-[(2,2,2-trifluoroacetyl)amino]benzenesulfonyl chloride (6.0 g, 21 mmol, 79% yield) as an off-white solid. ¹H NMR: (400 MHz, CDCl₃) δ8.19-8.13 (brs, 1H), 8.11 (d, J=8.9 Hz, 2H), 7.89 (d, J=8.8 Hz, 2H).

### 25.7 Preparation of Compound 4

To a solution of compound 2 (261 mg, 1.00 mmol, TFA salt, 1.0 equiv) in DCM (4 mL) was added DIEA (388 mg, 3.01 mmol, 523 μL, 3.0 equiv) and compound 3 (576 mg, 2.01 mmol, 2.0 equiv). The mixture was stirred at 20° C. for 2 h. The reaction mixture was concentrated under reduced pressure to give a residue. The residue was purified by column chromatography (SiO₂, Petroleum ether/Ethyl acetate=2/1 to Ethyl acetate/Methanol=100/1). Compound 4 (200 mg, 391 mmol, 39% yield) was obtained as a yellow oil. LC-MS: MS (ES⁺): m/z=512.4 [M+H⁺].

### 25.8 Preparation of Compound 5

To a solution of compound 4 (200 mg, 391 μmol, 1.0 equiv) in MeOH (2 mL) was added KOH (44 mg, 782 μmol, 2.0 equiv) in H₂O (2 mE). The mixture was stirred at 20° C. for 10 h. The mixture was adjusted with 2 N HCl to pH=5-6. Then the mixture was concentrated under reduced pressure to give a residue. The crude product was triturated with DCM/Methanol=10/1 at 20° C. for 0.5 h. The mixture was filtered and the filtrate was concentrated under reduced pressure. Compound 5 (156 mg, 388 μmol, 99% yield) was obtained as a yellow oil. LC-MS: MS (ES⁺): m/z=402.0 [M+H⁺].

### 25.9 Preparation of Compound 7

To a solution of compound 5 (144 mg, 358 μmol, 1.0 equiv) and compound 6 (123 mg, 358 μmol, 1.0 equiv) in NMP (2 mL) was added HCl (12 M, 33 μL, 1.1 equiv). The mixture was stirred at 90° C. for 10 h. The reaction mixture was concentrated under reduced pressure to give a residue. The residue was purified by prep-HPLC (column: Phenomenex luna C18 150*40 mm* 15 um; mobile phase: [water (0.1%TFA)-ACN]; B%: 15%-45%, 11 min). Compound 7 (70 mg, 98 μmol, 27% yield, TFA salt) was obtained as a yellow solid. LC-MS: MS (ES⁺): m/z=712.1 [M+H⁺].

### 25.10 Preparation of Compound I-25

To a solution of compound 7 (120 mg, 98 μmol, 1.0 equiv, TFA salt) and compound 8 (55 mg, 98 μmol, 1.0 equiv, TFA salt) in DMF (1 mL) was added DIEA (38 mg, 295 μmol, 51 μL, 3.0 equiv) and HATU (56 mg, 147 μmol, 1.5 equiv). The mixture was stirred at 20° C. for 0.5 h. The reaction mixture was quenched by addition H₂O (0.1 mL), and concentrated under reduced pressure to give a residue. The residue was purified by prep-HPLC (column: Waters Xbridge 150*25 mm* Sum; mobile phase: [water(1OmM NH₄HCO₃)-ACN]; B%: 31%-61%, 9 min) to give desired compound (R,E)-4-amino-1-(1-(4-((2-(2-(2-(4-((5-bromo-4-((2-carbamoyl-3-fluorophenyl)amino)pyrimidin-2-yl)amino)phenylsulfonamido)ethoxy)ethoxy)ethyl) methylamino)but-2-enoyl)piperidin-3-yl)-N-(4-(2-(dimethylamino)-2-oxoethyl)-2,3-dimethylphenyl)-1H-pyrazolo[3,4-d]pyrimidine-3-carboxamide (42 mg, 31 μmol, 32% yield, 85% purity) as a white solid. ¹H NMR (400 MHz, CD₃OD): δ8.33 (d, J=8.6 Hz, 1H), 8.28-8.17 (m, 2H), 7.85-7.74 (m, 2H), 7.72-7.61 (m, 2H), 7.51-7.43 (m, 1H), 7.42-7.27 (m, 1H), 7.02-6.92 (m, 2H), 6.81 (m, 2H), 4.64-3.91 (m, 3H), 3.84-3.74 (m, 2H), 3.71-3.37 (m, 11H), 3.26-3.20 (m, 1H), 3.17-2.96 (m, 10H), 2.65-2.54 (m, 1H), 2.50-2.42 (m, 1H), 2.29-2.16 (m, 10H), 1.77-1.63 (m, 1H). LC-MS: MS (ES⁺): RT=2.277 min, m/z=1144.3 [M+H⁺]; LC-MS METHOD 10.

### Example 26—Synthesis of Compound I-26 (Synthesized from 2 below according to General Scheme F in a similar manner to Compound I-25)

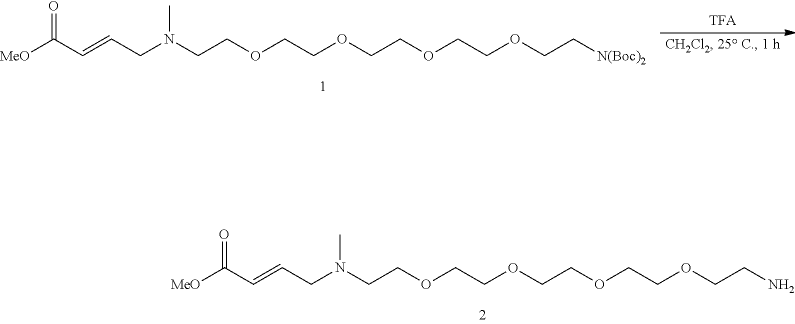

### 26.1 Preparation of Compound 2

To a solution of methyl E-4-[2-[2-[2-[2-[2-1[bis(tert-butoxycarbonyl)amino]ethoxy]ethoxy]ethoxy]ethoxy]ethyl-methyl-amino]but-2-enoate (612 mg, 1.12 mmol, 1.0 equiv) in CH₂Cl₂ (2 mL) was added TFA (1 mL), and then it was stirred at 25° C. for 1 h. The reaction mixture was concentrated to afford methyl €-4-[2-[2-[2-[2-(2-aminoethoxy)ethoxy]ethoxy]ethoxy]ethyl-methyl-amino]but-2-enoate (0.52 g, crude, TFA salt) was obtained as a colorless oil and used for the next step directly.

### 26.2 Preparation of Compound I-26

4-amino-[(3R)-1-[(E)-4-[2-[2-[2-[2-[2-[[4-[[5-bromo-4-(2-carbamoyl-3-fluoro-anilino) pyrimidin-2-yl]amino]phenyl]sulfonylamino]ethoxy]ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (23 mg, 18 μmol, 21% yield, HCl salt) as a yellow solid. LC-MS: MS (ES′): RT=2.639 min, m/z=1230.2, 1232.2 [M+H⁺], LC-MS METHOD 01. ¹H NMR: (400 MHz, CD₃OD) δ8.50-8.43 (m, 1H), 8.39-8.29 (m, 1H), 7.90 (d, J=8.6 Hz, 1H), 7.79 (d, J=8.6 Hz, 1H), 7.69 (d, J=8.3 Hz, 1H), 7.64-7.57 (m, 2H), 7.55-7.44 (m, 1H), 7.37-7.27 (m, 1H), 7.25-7.15 (m, 1H), 7.14-7.07 (m, 1H), 7.06-6.98 (m, 1H), 6.82-6.68 (m, 1H), 5.09-4.40 (m, 3H), 4.18-3.90 (m, 3H), 3.89-3.80 (m, 4H), 3.74-3.45 (m, 16H), 3.16 (s, 3H), 3.05-3.02 (m, 1H), 3.01 (s, 3H), 2.97 (t, J=4.6 Hz, 1H), 2.94 (s, 3H), 2.52-2.29 (m, 2H), 2.23 (d, J=10.4 Hz, 6H), 2.15-2.01 (m, 1H), 1.89-1.67 (m, 1H).

### Example 27—Synthesis of Compound I-27

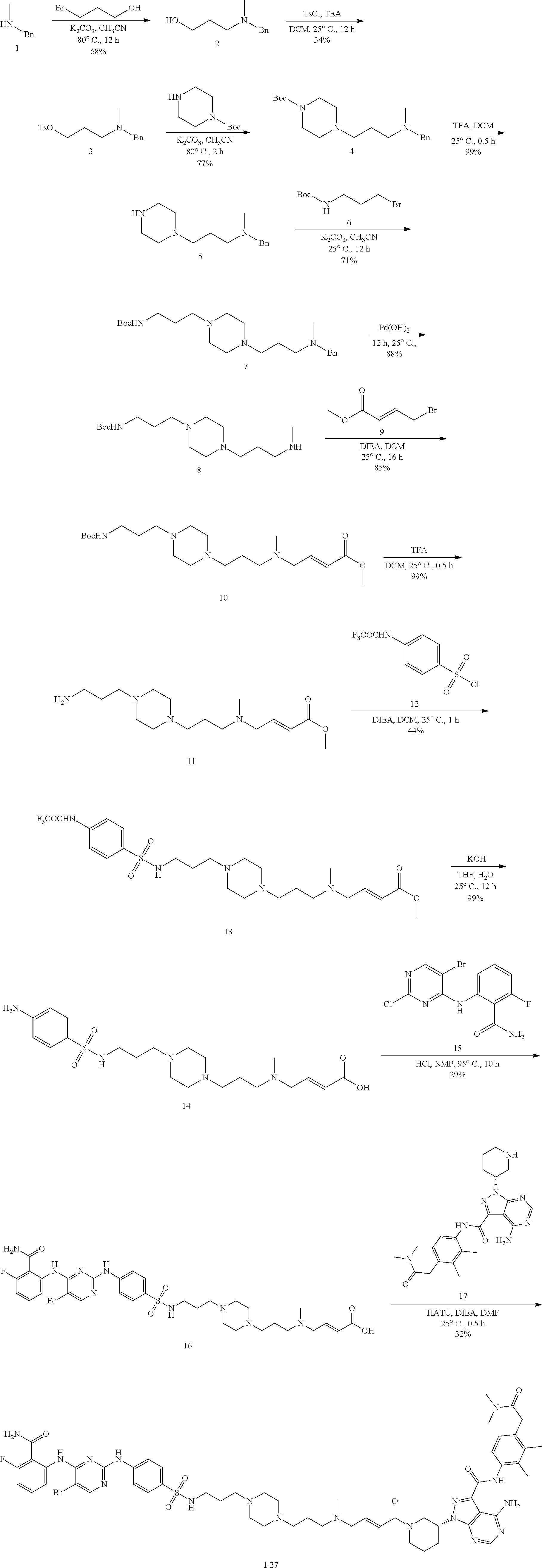

### 27.1 Preparation of Compound 2

To a solution of N-methyl-1-phenyl-methanamine (15.0 g, 124 mmol, 16.0 mL, 1.0 equiv) in MeOH (100 mL) was added 3-bromopropan-1-ol (22.37 g, 160.92 mmol, 14.52 mL, 1.3 equiv), K₂CO₃ (22.24 g, 160.92 mmol, 1.3 equiv). The mixture was stirred at 80° C. for 12 h. The reaction mixture was filtered and the filtrate was concentrated to afford crude product. The crude product was purified by was purified by chromatography (SiO₂, PE: EA=1:1 to 0:1, EA: MeOH=10:1 to 5:1) to give desired product 3-[benzyl(methyl)amino]propan-1-ol (15 g, 83.68 mmol, 68% yield) as a colorless oil. LC-MS: MS (ES⁺): m/z=180.0 [M+H⁺].

### 27.2 Preparation of Compound 3

To a solution of 3-[benzyl(methyl)amino]propan-1-ol (9.35 g, 52.2 mmol, 1.0 equiv) in DCM (100 mL) was added TEA (10.56 g, 104.3 mmol, 14.52 mL, 2.0 equiv), TsCl (7.36 g, 104 mmol, 2.0 equiv) at 0° C. The mixture was stirred at 25° C. for 12 h. The mixture was purified by column chromatography (SiO₂, PE: EA=5:1 to 1:1) to give the desired product 3-[benzyl(methyl)amino]propyl 4-methylbenzenesulfonate (5.91 g, 28.4 mmol, 34% yield) as a colorless oil. LC-MS: MS (ES⁺): m/z=334.2 [M+H⁺].

### 27.3 Preparation of Compound 4

To a solution of 3-[benzyl(methyl)amino]propyl 4-methylbenzenesulfonate (5.82 g, 17.4 mmol, 1.0 equiv), tert-butyl piperazine-1-carboxylate (3.25 g, 17.4 mmol, 1.0 equiv) in CH₃CN (20 mL) was added K₂CO₃ (7.24 g, 52.4 mmol, 3.0 equiv). The mixture was stirred at 80° C. for 2 h. The mixture was purified by column chromatography (SiO₂, PE: EA=2:1 to 1:1, EA:MeOH=10:1 to 4:1) to give a product tert-butyl 4-[3-[benzyl(methyl)amino]propyl]piperazine-1-carboxylate (4.70 g, 13.5 mmol, 77% yield) as a colorless oil. LC-MS: MS (ES⁺): m/z=348.3 [M+H⁺]. ¹H NMR (400 MHz, CDCl₃) δ7.33 (m, 5H), 3.64-3.49 (m, 2H), 3.47-3.38 (m, 4H), 2.52-2.33 (m, 8H), 2.30-2.19 (m, 3H), 1.83-1.69 (m, 2H), 1.48 (s, 9H).

### 27.4 Preparation of Compound 5

To a solution of tert-butyl 4-[3-[benzyl(methyl)amino]propyl]piperazine-1-carboxylate (2.3 g, 6.6 mmol, 1.0 equiv) in DCM (20 mL) was added TFA (15.4 g, 135 mmol, 10.0 mL, 20.4 equiv), it was stirred at 25° C. for 0.5 h. The reaction mixture was concentrated to afford desired product N-benzyl-N-methyl-3-piperazin-1-yl-propan-1-amine (4.78 g, 13.2 mmol, 99% yield, TFA salt) as a colorless oil. LC-MS: MS (ES⁺): m/z=248.2 [M+H⁺].

### 27.5 Preparation of Compound 7

To a solution of N-benzyl-N-methyl-3-piperazin-1-yl-propan-1-amine (2.39 g, 6.61 mmol, 1.0 equiv, TFA salt), tert-butyl N-(3-bromopropyl) arbamate (3.94 g, 16.5 mmol, 2.5 equiv) in CH₃CN (20 mL) was added K₂CO₃ (4.57 g, 33.1 mmol, 5.0 equiv), it was stirred at 25° C. for 12 h. The reaction mixture was diluted with water (100 mL), extracted with EtOAc (2×100 mL). The combined organic layers were dried over anhydrous Na₂SO₄, filtered and concentrated to give the crude product. The crude product was purified by column chromatography (SiO₂, EA: MeOH=10:1 to 4:1) first, then it was purified by prep-HPLC(column: Phenomenex Synergi Max-RP 250*50 mm*10 um; mobile phase: [water(0.225% FA)-ACN]; B%: 1OACN %-40ACN %, 11 min) to give the desired product tert-butyl N-[3-[4-[3-[benzyl(methyl)amino]propyl]piperazin-1-yl]propyl]carbamate (3.8 g, 9.4 mmol, 71.% yield) as a colorless oil. ¹H NMR (400 MHz, CDCl3) δ (ppm) 7.28-7.19 (m, 5H), 3.42 (s, 2H), 3.12 (brd, J=5.6 Hz, 2H), 2.54-2.24 (m, 14H), 2.13 (s, 3H), 1.71-1.59 (m, 4H), 1.37 (s, 9H). LC-MS: MS (ES⁺): m/z=348.3 [M+H⁺].

### 27.6 Preparation of Compound 8

To a solution of tert-butyl N-[3-[4-[3-[benzyl(methyl)amino]propyl]piperazin-1-yl]propyl]carbamate (1.9 g, 4.7 mmol, 1.0 equiv) in TFE (20 mL) was added Pd(OH)₂ (380.0 mg, 270.6 μmol, 10% purity, 13.6 equiv). The mixture was stirred under hydrogen balloon at 25° C. for 12 h. The reaction mixture was filtered and the filtrate was concentrated to give desired product tert-butyl N-[3-[4-[3-(methylamino)propyl]piperazin-1-yl]propyl]carbamate (1.3 g, 4.1 mmol, 88% yield) as a black brown oil. ¹H NMR (400 MHz, CDCl₃) δ3.95 (q, J=8.7 Hz, 2H), 3.19 (br d, J=5.4 Hz, 2H), 2.69-2.29 (m, 15H), 1.77-1.66 (m, 4H), 1.44 (s, 9H).

### 27.7 Preparation of Compound 10

To a solution of tert-butyl N-[3-[4-[3-(methylamino)propyl]piperazin-1-yl]propyl]carbamate (800 mg, 2.50 mmol, 1.0 equiv), methyl (E)-4-bromobut-2-enoate (409.8 mg, 2.290 mmol, 269.6 μL, 0.9 equiv) in DCM (12 mL) was added DIEA (493 mg, 3.82 mmol, 664 μL, 1.5 equiv), it was stirred at 25° C. for 16 h. The reaction mixture was purified by column chromatography (SiO₂, EA: MeOH=1:0 to 5:1) to give the desired mass product methyl (E)-4-[3-[4-[3-(tert-butoxycarbonylamino)propyl]piperazin-1-yl]propyl-methyl-amino]but-2-enoate (893 mg, 2.16 mmol, 85% yield) as a yellow oil. LC-MS: MS (ES⁺): m/z=413.6 [M+H⁺].

### 27.8 Preparation of Compound 11

To a solution of methyl (E)-4-[3-[4-[3-(tert-butoxycarbonylamino)propyl]piperazin-1-yl]propyl-methyl-amino]but-2-enoate (250 mg, 605 μmol, 1.0 equiv) in DCM (1 mL) was added TFA (1 mL), it was stirred at 25° C. for 0.5 h. The reaction mixture was concentrated to afford a product methyl (E)-4-[3-[4-(3-aminopropyl)piperazin-1-yl]propyl-methyl-amino]but-2-enoate (258.4 mg, 605.9 μmol, 99% yield, TFA salt) as a black brown oil. LC-MS: MS (ES⁺): m/z=313.3 [M+H⁺].

### 27.9 Preparation of Compound 13

To a solution of methyl (E)-4-[3-[4-(3-aminopropyl)piperazin-1-yl]propyl-methyl-amino]but-2-enoate (258 mg, 604 μmol, 1.0 equiv TFA salt) in DCM (5 mL) was added DIEA (234 mg, 1.81 mmol, 316 μL, 3.0 equiv) and 4-[(2,2,2-trifluoroacetyl)amino]benzenesulfonyl chloride (261.02 mg, 907.44 μmol, 1.5 equiv), it was stirred at 25° C. for 2 h. The mixture was diluted with water (50 mL), extracted with EtOAc (2×50 mL), dried, filtered and concentrated to afford a crude product. The crude product was purified by prep-HPLC (column: Phenomenex Gemini-NX C18 75*30 mm*3 um; mobile phase: [water(0.225% FA)-ACN]; B%: 1%-25%, 7 min) to afford desired product methyl (E)-4-[methyl-[3-[4-[3-[[4-[(2,2,2-trifluoroacetyl)amino]phenyl]sulfonylamino]propyl]piperazin-1-yl]propyl]amino]but-2-enoate (153 mg, 271 μmol, 44% yield) as a light yellow solid. LC-MS: MS (ES⁺): m/z=564.2 [M+H⁺].

### 27.10 Preparation of Compound 14

To a mixture of methyl (E)-4-[methyl-[3-1[4-[3-[1[4-1[(2,2,2-trifluoroacetyl)amino]phenyl]sulfonylamino]propyl]piperazin-1-yl]propyl]amino]but-2-enoate (110 mg, 195 μmol, 1.0 equiv) in THF (2 mL) and H₂O (2 mL) was added KOH (1.0 M, 1.9 mL, 10.0 equiv). The mixture was stirred at 20° C. for 12 h. 1 N HCl was added to acidify the solution (pH=5-6). The mixture was concentrated to give a residue. The residue was diluted with DCM/MeOH=10/1 (30 mL) and the solution was stirred for 0.5 h. Then the solution was filtered and the filtrate was concentrated to give the desired product (E)-4-[3-[4-[3-[(4-aminophenyl)sulfonylamino]propyl]piperazin-1-yl]propyl-methyl-amino]but-2-enoic acid (100 mg,crude) as a yellow solid. LC-MS: MS (ES⁺): m/z=454.2 [M+H⁺].

### 27.11 Preparation of Compound 16

To a solution of (E)-4-[3-[4-[3-[(4-aminophenyl)sulfonylamino]propyl]piperazin-1-yl]propyl-methyl-amino]but-2-enoic acid (100 mg, 220 μmol, 1.0 equiv), 2-[(5-bromo-2-chloro-pyrimidin-4-yl)amino]-6-fluoro-benzamide (76.18 mg, 220.4 μmol, 1.0 equiv) in NMP (3 mL) was added HCl (267.94 mg, 881.84 μmol, 262.69 μL, 12% purity, 4.0 equiv), it was stirred at 95° C. for 10 h. The reaction mixture was purified by prep-HPLC(column: 3_Phenomenex Luna C18 75*30 mm*3 um; mobile phase: [water(0.1%TFA)-ACN]; B%: 18%-38%, 7 min) to give the desired product (E)-4-[3-[4-[3-[[4-[[5-bromo-4-(2-carbamoyl-3-fluoro-anilino)pyrimidin-2-yl]amino]phenyl]sulfonylamino]propyl]piperazin-1-yl]propyl-methyl-amino]but-2-enoic acid (50 mg, 65 μmol, 29% yield) as a yellow solid. LC-MS: MS (ES⁺): m/z=764.3 [M+H⁺], 382.6[M/2+H⁺].

### 27.12 Preparation of Compound I-27

To a solution of (E)-4-[3-[4-[3-[[4-[[5-bromo-4-(2-carbamoyl-3-fluoro-anilino)pyrimidin-2-yl]amino]phenyl]sulfonylamino]propyl]piperazin-1-yl]propyl-methyl-amino]but-2-enoic acid (43.50 mg, 57.04 μmol, 1.0 equiv) and 4-amino-N-[4-[2-(dimethylamino)-2-oxo-ethyl]-2,3-dimethyl-phenyl]-1-[(3R)-3-piperidyl]pyrazolo[3,4-d]pyrimidine-3-carboxamide (32.2 mg, 57.0 μmol, 1.0 equiv, TFA) in DMF (1 mL) was added DIEA (36.86 mg, 285.1 μmol, 49.67 μL, 5.0 equiv) and HATU (32.53 mg, 85.55 μmol, 1.5 equiv), it was stirred at 25° C. for 0.5 h. The reaction mixture was quenched by 0.1 mL water and it was purified by prep-HPLC(column: Phenomenex Gemini-NX C18 75*30 mm*3 um; mobile phase: [water(0.225% FA)-ACN]; B%: 12%-42%, 7 min) to give a desired product (1S)-4-amino-1-[1-[(E)-4-[3-[4-[3-[[4-[[5-bromo-4-(2-carbamoyl-3-fluoro-anilino)pyrimidin-2-yl]amino]phenyl]sulfonylamino]propyl]piperazin-1-yl]propyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (23.35 mg, 18.81 μmol, 32% yield, FA) as a white solid. ¹H NMR (400 MHz, CD₃OD) δ (ppm) 8.38 (d, J=8.4 Hz, 1H), 8.31-8.21 (m, 2H), 7.85 (br d, J=8.8 Hz, 2H), 7.76-7.69 (m, 2H), 7.56-7.48 (m, 1H), 7.45-7.34 (m, 1H), 7.06-6.95 (m, 2H), 6.76 (br s, 1H), 6.69-6.56 (m, 1H), 6.51-6.38 (m, 1H), 6.70-6.38 (m, 1H), 6.43 (br d, J=15.2 Hz, 1H), 5.08-5.01 (m, 1H), 4.70-3.88 (m, 1H), 3.76-3.61 (m, 1H), 3.59-3.36 (m, 1H), 3.20-2.87 (m, 1H), 2.79-2.02 (m, 1H), 1.85-1.56 (m, 1H). LC-MS: MS (ES⁺): RT=2.173 min, m/z=1196.4 [M+H⁺]. LCMS METHOD 10.

### Example 28—Synthesis of Compound I-28

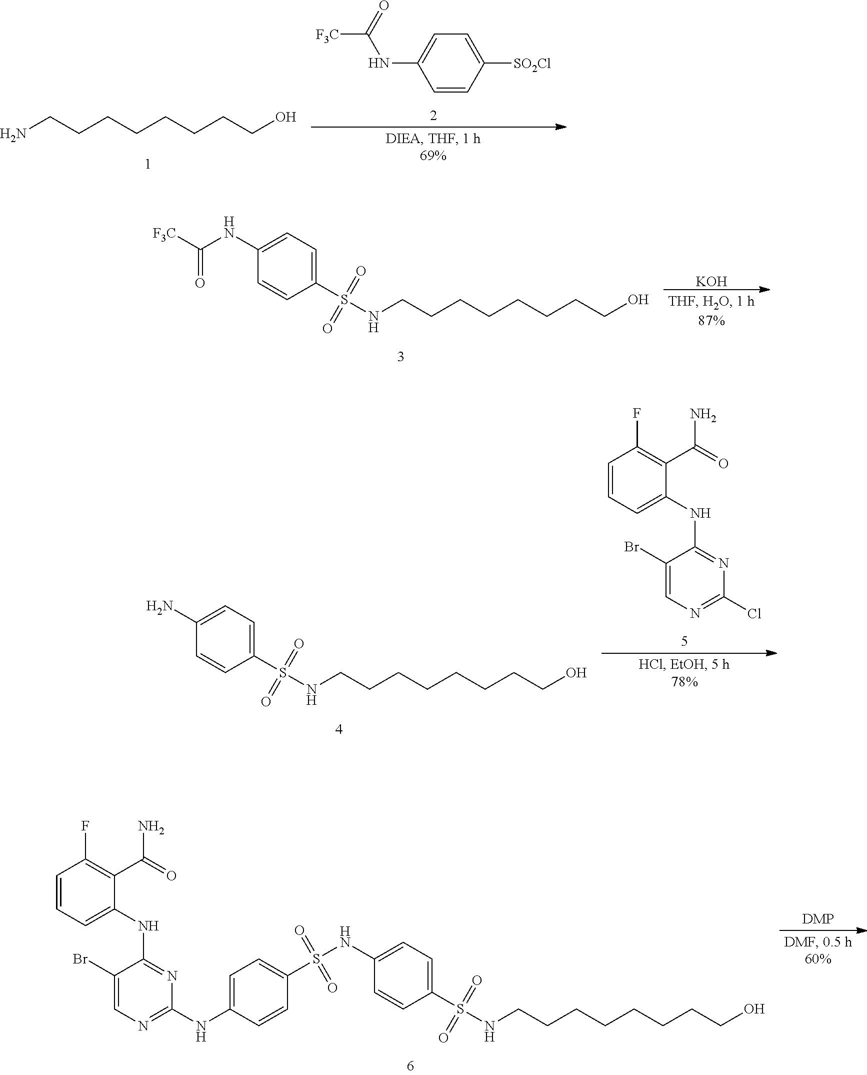

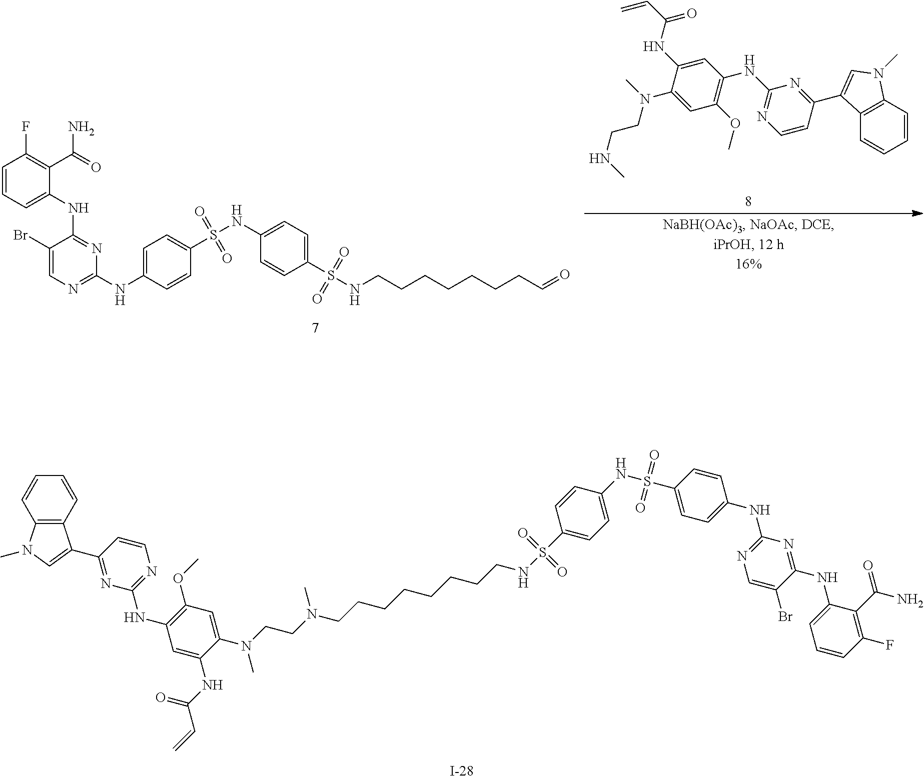

### 28.1 Preparation of Compound 3

To a solution of 8-aminooctan-1-ol (1.0 g, 6.9 mmol, 1.0 equiv) in THF (20 mL) was added DIEA (2.7 g, 21 mmol, 3.0 equiv) and 4-[(2,2,2-trifluoroacetyl)amino]benzenesulfonyl chloride (2.0 g, 6.9 mmol, 1.0 equiv). The mixture was stirred at 20° C. for 1 h and concentrated. The residue was purified by column chromatography (SiO₂, Petroleum ether/Ethyl acetate=5/1 to 0/1) to give 2,2,2-trifluoro-N-[4-(8-hydroxyoctylsulfamoyl)phenyl]acetamide (1.9 g, 69% yield) as an off-white solid.

### 28.2 Preparation of Compound 4

To a solution of 2,2,2-trifluoro-N-[4-(8-hydroxyoctylsulfamoyl)phenyl]acetamide (1.5 g, 3.8 mmol, 1.0 equiv) in H₂O (7 mL) and THF (15 mL) was added KOH (2.1 g, 38 mmol, 10.0 equiv). The mixture was stirred at 20° C. for 1 h and quenched with water (20 mL). The mixture was extracted with EtOAc (3×30 mL), dried over Na₂SO₄, filtered and concentrated to give the desired product 4-amino-N-(8-hydroxyoctyl)benzenesulfonamide (1.0 g, 87% yield) as a white solid. LC-MS: MS (ES⁺): m/z=301.4 [M+H⁺].

### 28.3 Preparation of Compound 6

To a solution of 4-amino-N-(8-hydroxyoctyl)benzenesulfonamide (500 mg, 2 mmol, 1.0 equiv) and 2-[(5-bromo-2-chloro-pyrimidin-4-yl)amino]-6-fluoro-benzamide (575 mg, 2 mmol, 1.0 equiv) in EtOH (10 mL) was added aq.HCl (12 M, 69 μL, 0.5 equiv). The mixture was stirred at 80° C. for 6 h and concentrated. The residue was triturated with MeOH (30 mL) at 20° C. for 30 min to give 2-[[5-bromo-2-[4-(8-hydroxyoctylsulfamoyl)anilino]pyrimidin-4-yl]amino]-6-fluoro-benzamide (800 mg, 78% yield) as a white solid. ¹H NMR (400 MHz, DMSO-d₆): δ10.16 (s, 1H), 10.01 (s, 1H), 8.41 (s, 1H), 8.25-8.13 (m, 3H), 7.84 (d, 2H, J=8.8 Hz), 7.63 (d, 2H, J=8.8 Hz), 7.56-7.49 (m, 1H), 7.39 (s, 1H), 7.13-7.08 (m, 1H), 3.34 (t, 2H, J=6.4 Hz), 2.69-2.65 (m, 2H), 1.39-1.30 (m, 4H), 1.23-1.15 (m, 8H).

### 28.4 Preparation of Compound 7

To a solution of 2-[[5-bromo-2-[4-(8-hydroxyoctylsulfamoyl)anilino]pyrimidin-4-yl]amino]-6-fluoro-benzamide (100 mg, 164 μmol, 1.0 equiv) in DMF (1.5 mL) was added Dess-Martin reagent (104 mg, 246 μmol, 1.5 equiv). The mixture was stirred at 20° C. for 0.5 h and purified by prep-TLC (DCM/MeOH=10/1) to give 2-[[5-bromo-2-[4-(8-hydroxyoctylsulfamoyl)anilino]pyrimidin-4-yl]amino]-6-fluoro-benzamide (60 mg, 60% yield) as a yellow solid. LC-MS: MS (ES⁺): m/z=609.0 [M+H⁺].

### 28.5 Preparation of Compound I-28

To a solution 2-[[5-bromo-2-[4-(8-oxooctylsulfamoyl)anilino]pyrimidin-4-yl]amino]-6-fluoro-benzamide (60 mg, 98 μmol, 1.0 equiv), N-[4-methoxy-5-[[4-(1-methylindol-3-yl)pyrimidin-2-yl]amino]-2-[methyl-[2-(methylamino)ethyl]amino]phenyl]prop-2-enamide (88 mg, 148 μmol, 1.5 equiv, TFA salt) in DCE (5 mL) and IPA (5 mL) was added NaOAc (24 mg, 296 μmol, 3.0 equiv) and NaBH(OAc)₃ (104 mg, 493 μmol, 5.0 equiv). The mixture was stirred at 20° C. for 12 h and purified by prep-HPLC (column: Phenomenex Synergi C18 150*25 mm* 10%m; mobile phase: [water(0.225% FA)-ACN]; B%: 20%-50%, 10 min) to give the desired product 2-[[5-bromo-2-[4-[8-[2-[5-methoxy-N-methyl-4-[[4-(1-methylindol-3-yl)pyrimidin-2-yl]amino]-2-(prop-2-enoylamino)anilino]ethyl-methyl-amino]octylsulfamoyl]anilino]pyrimidin-4-yl]amino]-6-fluoro-benzamide (18 mg, 16% yield) as a light yellow solid. ¹H NMR (400 MHz, DMSO-d₆): δ10.40-10.10 (m, 1H), 9.95-9.84 (m, 2H), 9.09 (s, 1H), 8.65 (s, 1H), 8.39 (s, 1H), 8.33-8.30 (m, 2H), 8.27-8.22 (m, 2H), 8.17 (s, 1H), 8.11 (s, 1H), 7.89-7.82 (m, 3H), 7.64-7.61 (m, 2H), 7.54-7.49 (m, 2H), 7.33 (t, 1H, J=5.6 Hz), 7.22 (d, 2H, J=5.2 Hz), 7.16-7.06 (m, 2H), 7.00 (s, 1H), 6.51-6.41 (m, 1H), 6.28-6.21 (m, 1H), 5.74 (d, 1H, J=10.0 Hz), 3.91-3.85 (m, 7H), 2.69-2.67 (m, 5H), 2.36 (d, 2H, J=5.6 Hz), 2.32-2.28 (m, 2H), 2.16 (s, 3H), 1.37-1.26 (m, 4H), 1.20-1.12 (m, 8H). LC-MS: MS (ES⁺): RT=2.119 min, m/z=1078.4 [M+H⁺], LC-MS METHOD 25.

### Example 29—Synthesis of Compound I-29

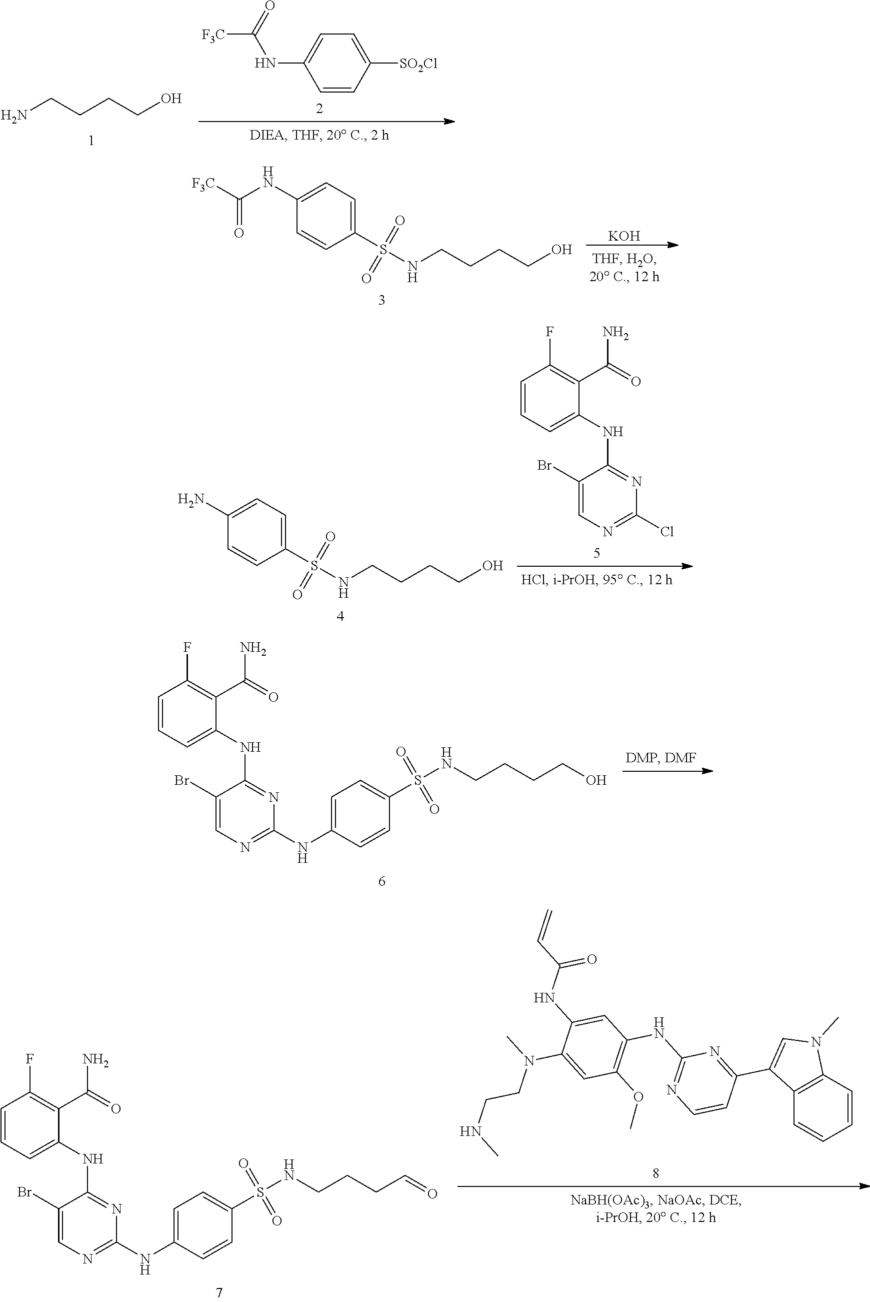

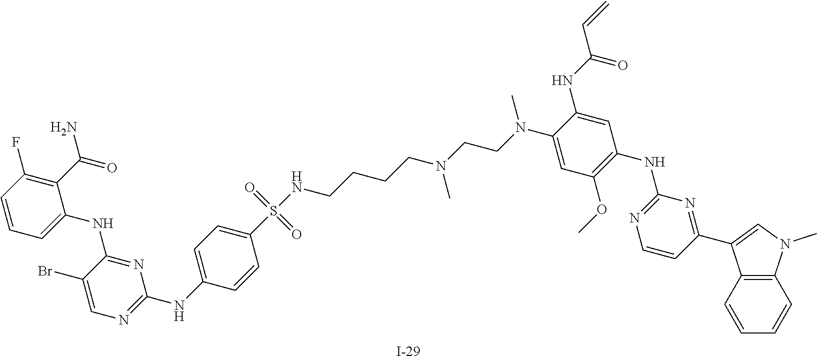

### 29.1 Preparation of Compound 3

To a solution of 4-aminobutan-1-ol (500 mg, 5.6 mmol, 1 equiv) and DIEA (2.17 g, 16.8 mmol, 3 equiv) in THF (50 mL) was added 4-[(2,2,2-trifluoroacetyl)amino]benzenesulfonyl chloride (1.77 g, 6.2 mmol, 1.1 equiv). The mixture was stirred at 20° C. for 2 h and purified by column chromatography (SiO₂, Petroleum ether/Ethyl acetate=10/1 to 1/1) to give 2, 2, 2-trifluoro-N-[4-(4-hydroxybutylsulfamoyl) phenyl]acetamide (1.5 g, 78% yield) as a white solid. ¹H NMR (400 MHz, DMSO-d₆) δ11.60 (s, 1H), 7.95-7.73 (m, 4H), 7.63-7.52 (m, 1H), 4.39-4.36 (m, 1H), 3.34 (s, 2H), 2.79-2.67 (m, 2H), 1.44-1.31 (m, 4H). LC-MS: MS (ES⁺): m/z=341.3[M+H⁺].

### 29.2 Preparation of Compound 4

To a solution of 2, 2, 2-trifluoro-N-[4-(4-hydroxybutylsulfamoyl) phenyl]acetamide (1.5 g, 4.4 mmol, 1 equiv) in THF (10 mL) and H₂O (5 mL) was added KOH (2.47 g, 44 mmol, 10 equiv). The mixture was stirred at 20° C. for 12 h. The mixture was added 3 M (HCl, 5 ml) to adjusted the pH=5 and extracted with EtOAc (3×200 mL). The organic layer was concentrated and purified by column chromatography (SiO₂, Petroleum ether/Ethyl acetate=10/1 to 1/1) to give 4-amino-N-(4-hydroxybutyl)benzenesulfonamide (1 g, 92% yield) as a brown oil. LC-MS: MS (ES⁺): m/z=283.1 [M+K+].

### 29.3 Preparation of 6

To a solution of 4-amino-N-(4-hydroxybutyl)benzenesulfonamide (400 mg, 1.64 mmol, 1 equiv) and 2-[(5-bromo-2-chloro-pyrimidin-4-yl)amino]-6-fluoro-benzamide (565 mg, 1.64 mmol, 1 equiv) in i-PrOH (5 mL) was added HCl (12 M, 136 μL, 1 equiv). The mixture was stirred at 95° C. for 12 h and purified by prep-HPLC (column: Phenomenex luna C18 150*40 mm* 15 um; mobile phase: [water (0.225% FA) -ACN]; B%: 26%-56%, 10 min, column: Unisil 3-100 C18 Ultra 150*50 mm*3 um; mobile phase: [water (0.225% FA) -ACN]; B%: 30%-60%, 10 min) to give the desired product 2-[[5-bromo-2-[4-(4-hydroxybutylsulfamoyl) anilino]pyrimidin-4-yl]amino]-6-fluoro-benzamide (400 mg, 44% yield) as a white solid. ¹H NMR (400 MHz, DMSO-d₆) δ10.10 (s, 1H), 9.95 (s, 1H), 8.39 (s, 1H), 8.24-8.08 (m, 3H), 7.85-7.82 (m, 2H), 7.62-7.60 (m, 2H), 7.57-7.47 (m, 1H), 7.37-7.34 (m, 1H), 7.11-7.09 (m, 1H), 4.41-4.30 (m, 2H), 3.52-3.45 (m, 2H), 1.37-1.32 (m, 4H). LC-MS: MS (ES⁺): m/z=555.1 [M+H⁺].

### 29.4 Preparation of 7

To a solution of 2-[[5-bromo-2-[4-(4-hydroxybutylsulfamoyl) anilino]pyrimidin-4-yl]amino]-6-fluoro-benzamide (100 mg, 180 μmol, 1 equiv) in DMF (2 mL) was added Dess-Martin reagent (114 mg, 271 μmol, 1.5 equiv). The mixture was stirred at 20° C. for 1 h. The mixture was diluted with water (20 mL) and extracted with DCM (3×20 mL). The combined organic layers were washed with brine (20 mL), dried over Na₂SO₄, filtered and concentrated. The residue was purified by prep-TLC (SiO₂, DCM: MeOH=10:1) to give the desired product 2-[[5-bromo-2-[4-(4-oxobutylsulfamoyl) anilino]pyrimidin-4-yl]amino]-6-fluoro-benzamide (50 mg, 50% yield) as a yellow oil. LC-MS: MS (ES⁺): m/z=555.1 [M+H⁺].

### 29.5 Preparation of Compound I-29

To a solution of 2-[[5-bromo-2-[4-(4-oxobutylsulfamoyl)anilino]pyrimidin-4-yl]amino]-6-fluoro-benzamide (50 mg, 90 μmol, 1 equiv) and N-[4-methoxy-5-[[4-(1-methylindol-3-yl)pyrimidin-2-yl]amino]-2-[methyl-[2-(methylamino)ethyl]amino]phenyl]prop-2-enamide (54 mg, 90 μmol, 1.00 equiv, TFA salt) in DCE (1 mL) and i-PrOH (1 mL) was added NaBH(OAc)₃ (96 mg, 453 μmol, 5 equiv) and NaOAc (22 mg, 272 μmol, 3 equiv). The mixture was stirred at 20° C. for 12 h and purified by prep-HPLC (column: Unisil 3-100 C18 Ultra 150*50 mm*3 um; mobile phase: [water (0.225% FA) -ACN]; B%: 16%-46%, 10 min) to give 2-[[5-bromo-2-[4-[4-[2-[5-methoxy-N-methyl-4-[[4-(1-methylindol-3-yl)pyrimidin-2-yl]amino]-2-(prop-2-enoylamino) anilino]ethyl-methyl-amino]butylsulfamoyl]anilino]pyrimidin-4-yl]amino]-6-fluoro-benzamide (17 mg, 18% yield) as a yellow solid. ¹H NMR (400 MHz, DMSO-d₆) δ10.12 (s, 1H), 9.93 (s, 1H), 9.77 (s, 1H), 9.08 (s, 1H), 8.63 (s, 1H), 8.37-8.14 (m, 5H), 7.93-7.76 (m, 3H), 7.66-7.37 (m, 5H), 7.25-6.94 (m, 5H), 6.56-6.39 (m, 1H), 6.29-6.16 (m, 1H), 5.80-5.66 (m, 1H), 3.93-3.81 (m, 6H), 2.84-2.83 (m, 2H), 2.68 (s, 4H), 2.41-2.29 (m, 6H), 2.14 (s, 3H), 1.38-1.25 (m, 4H). LC-MS: MS (ES⁺): RT=2.505 min, m/z=1022.2, 1023.2 [M+H⁺], LC-MS METHOD 01.

### Example 30—Synthesis of Compound I-30

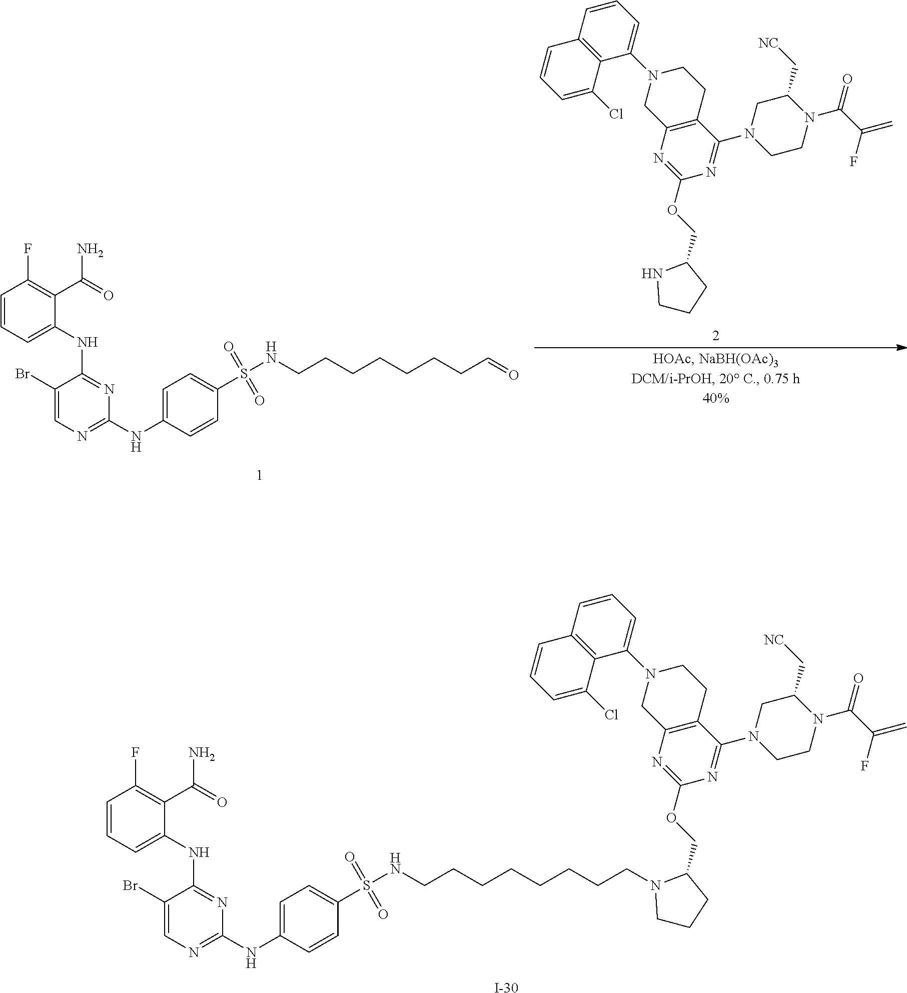

A mixture of compound 2 (62 mg, 88 μmol, 1.0 equiv, TFA salt) and NaOAc (36 mg, 438 μmol, 5.0 equiv) in DCM (2 mL) and i-PrOH (2 mL) was stirred at 20° C. for 15 min. Compound 1 (53 mg, 87 μmol, 1.0 equiv) was added. Then NaBH(OAc)₃ (93 mg, 438 μmol, 5.0 equiv) was added. The mixture was stirred at 20° C. for 0.5 h. The reaction mixture was partitioned between brine (20 mL) and DCM (80 mL). The organic phase was separated, dried over [Na₂SO₄], filtered and concentrated under reduced pressure to give a residue, The residue was purified by prep-TLC (SiO₂, DCM: MeOH=10:1) to give desired compound 2-((5-bromo-2-((4-(N-(8-((S)-2-(((7-(8-chloronaphthalen-1-yl)-4-((S)-3-(cyanomethyl)-4-(2-fluoroacryloyl)piperazin-1-yl)-5,6,7,8-tetrahydropyrido [3,4-d]pyrimidin-2-yl)oxy)methyl) pyrrolidin-1-yl)octyl)sulfamoyl)phenyl)-amino)pyrimidin-4-yl)amino)-6-fluorobenzamide (42 mg, 35 μmol, 40% yield, 97% purity, CH₃COOH salt) was obtained as a yellow solid. ¹HNMR (400 MHz, CD₃OD): δ8.46-8.30 (m, 1H), 8.25-8.10 (m, 1H), 7.86-7.74 (m, 3H), 7.71-7.59 (m, 3H), 7.51-7.40 (m, 3H), 7.37-7.29 (t, J=7.8 Hz, 1H), 7.28-7.20 (t, J=6.4 Hz, 1H), 7.01-6.91 (t, J=12.2 Hz 1H), 5.41-5.23 (m, 2H), 4.64-4.06 (m, 6H), 3.69-3.40 (m, 5H), 3.30-2.92 (m, 8H), 2.90-2.60 (m, 5H), 2.29-2.13 (m, 1H), 2.09-1.81 (m, 4H), 1.69-1.51 (m, 2H), 1.43-1.08 (m, 12H). LC-MS: MS (ES⁺): RT=1.995 min, m/z=1180.3, 1182.3 [M+H⁺]; LC-MS METHOD 40.

### Example 31—Synthesis of Compound I-31

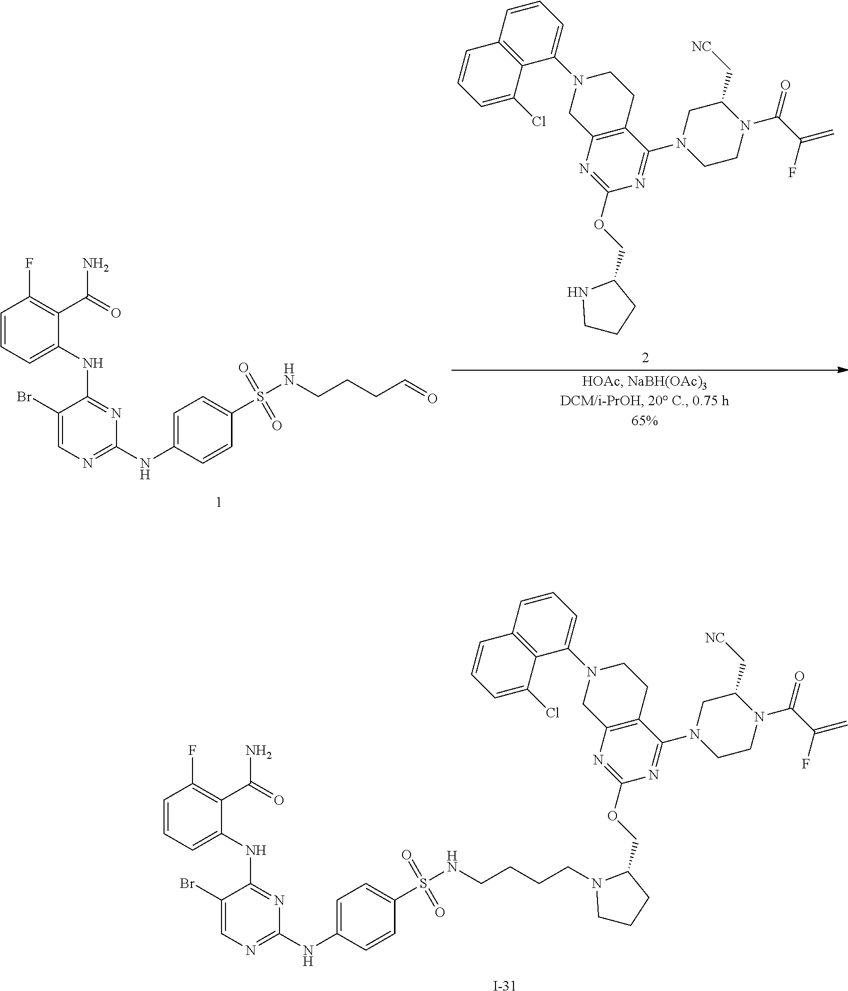

A mixture of compound 2 (40 mg, 56 μmol, 1.0 equiv, TFA salt) and NaOAc (24 mg, 292 μmol, 5.1 equiv) in DCM (1 mL) and i-PrOH (1 mL) was stirred at 20° C. for 15 min. Compound 1 (37 mg, 67 μmol, 1.1 equiv) was added, and hen NaBH(OAc)₃ (60 mg, 283 μmol, 4.9 equiv) was added. The mixture was stirred at 20° C. for 0.5 h. The reaction mixture was partitioned between brine (20 mL) and DCM (80 mL). The organic phase was separated, dried over Na₂SO₄, filtered and concentrated under reduced pressure to give a residue. The residue was purified by prep-HPLC (column: Phenomenex Synergi C18 150*25 mm* 10 um; mobile phase: [water(0.225% FA)-ACN]; B%: 24%-54%, 10 min) to give desired product 2-((5-bromo-2-((4-(N-(4-((S)-2-(((7-(8-chloronaphthalen-1-yl)-4-((S)-3-(cyanomethyl)-4-(2-fluoroacryloyl)piperazin-1-yl)-5,6,7,8-tetrahydropyrido [3,4-d]pyrimidin-2-yl)oxy)methyl)pyrrolidin-1-yl)butyl)sulfamoyl)phenyl)amino)-pyrimidin-4-yl)amino)-6-fluorobenzamide (43 mg, 37 μmol, 65% yield, 99% purity, FA salt) as a white solid. ¹HNMR (400 MHz, CD₃OD): δ8.56-8.43 (m, 1H), 8.39-8.29 (m, 1H), 8.27-8.17 (m, 1H), 7.79 (m, 3H), 7.66 (m, 3H), 7.55-7.41 (m, 3H), 7.39-7.21 (m, 2H), 7.03-6.89 (m, 1H), 5.40-5.23 (m, 2H), 4.63-4.00 (m, 6H), 3.77-3.34 (m, 8H), 3.29-3.05 (m, 5H), 3.04-2.57 (m, 5H), 2.33-2.13 (m, 1H), 2.09-1.83 (m, 3H), 1.80-1.62 (m, 2H), 1.61-1.42 (m, 2H). LC-MS: MS (ES⁺): RT=1.758 min, m/z=1126.3 [M+H⁺]; LC-MS METHOD 40.

### Example 32—Synthesis of Compound I-32 (Synthesized according to General Scheme G)

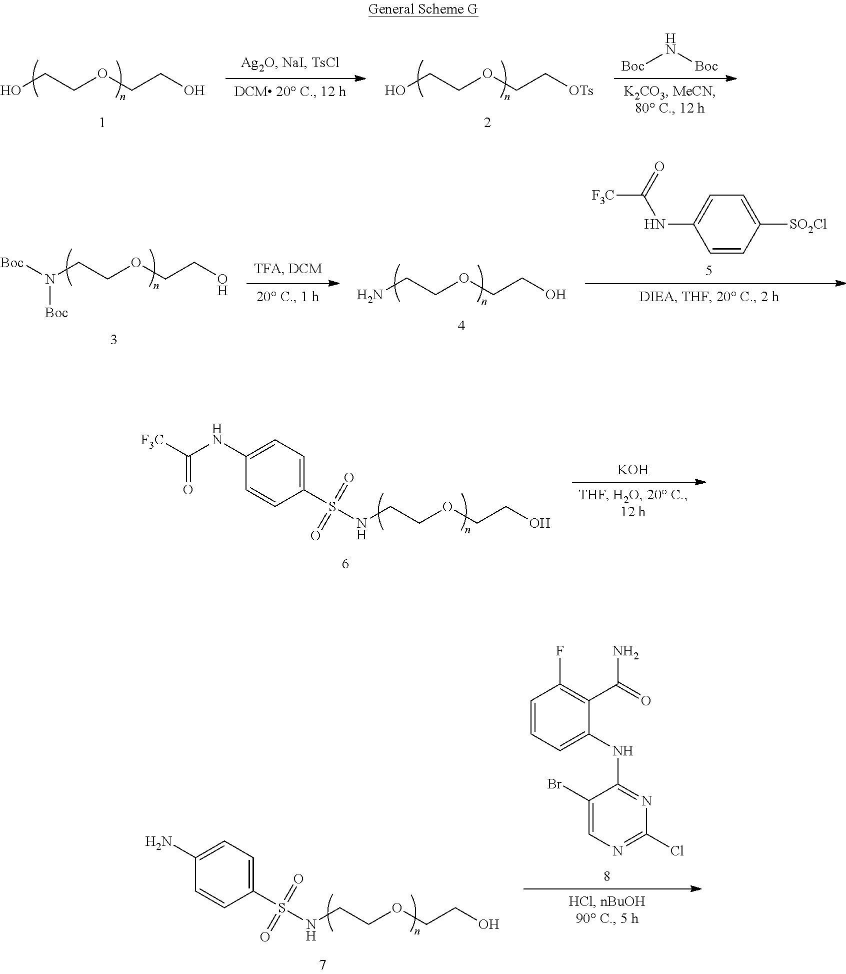

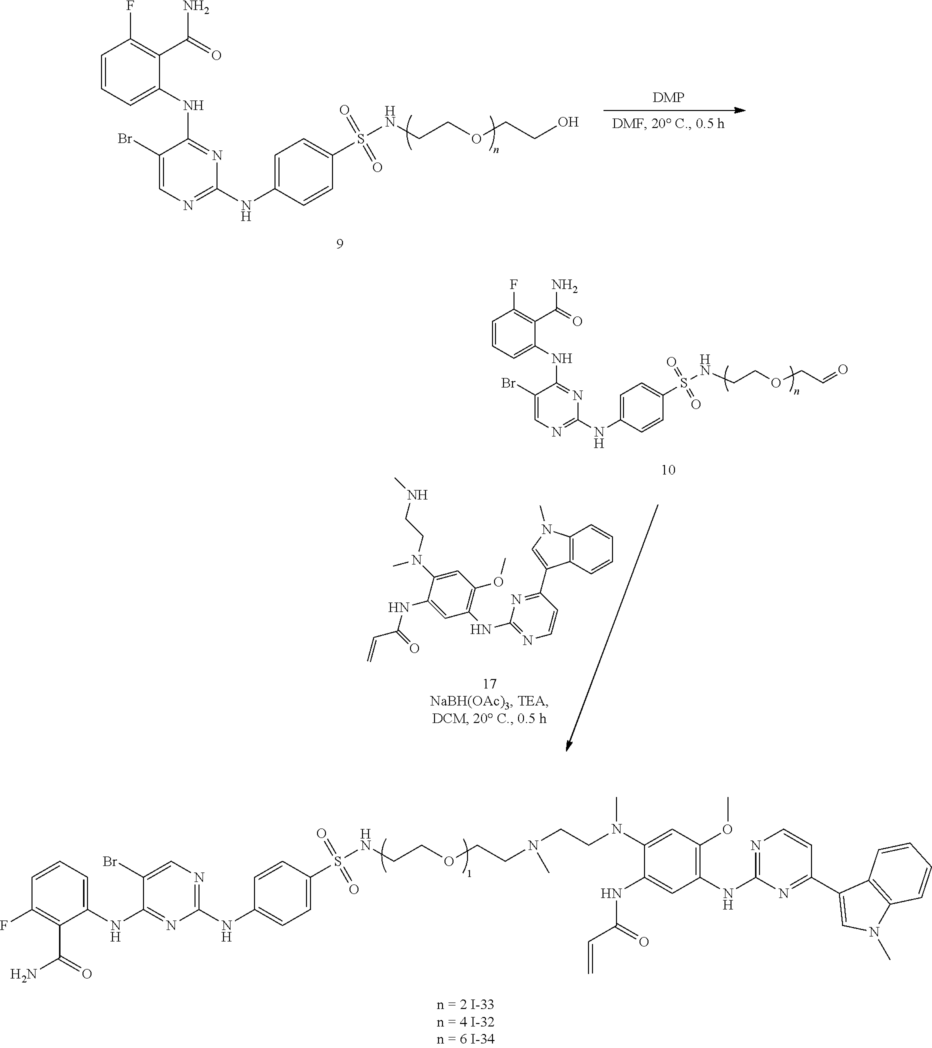

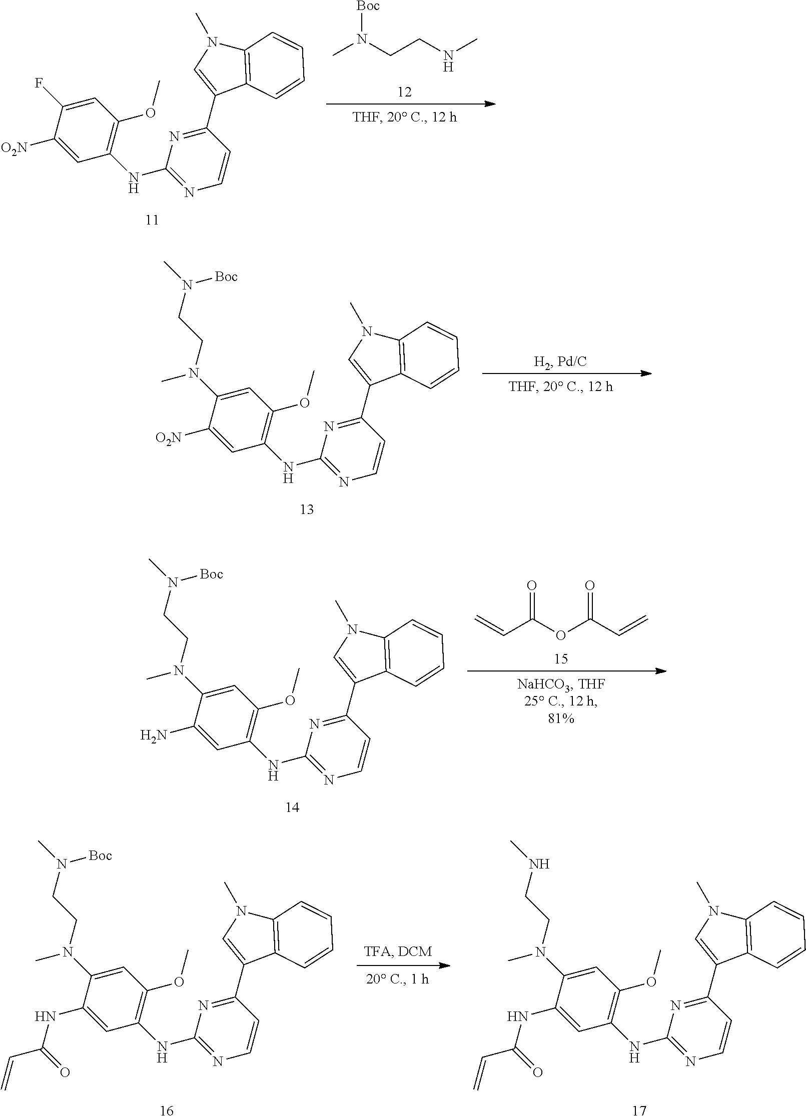

### 32.1 Preparation of Compound 2

To a solution of 2-[2-[2-[2-(2-hydroxyethoxy)ethoxy]ethoxy]ethoxy]ethanol (5 g, 21 mmol, 1 equiv) and TsCl (4.0 g, 21 mmol, 1 equiv) in DCM (50 mL) at 0° C. was added Ag₂O (7.29 g, 31.5 mmol, 1.5 equiv) and NaI (3.46 g, 23.1 mmol, 1.1 equiv). The mixture was stirred at 20° C. for 12 h and concentrated. The residue was purified by prep-HPLC (column: Phenomenex luna C18 250*50 mm*10 um; mobile phase: [water (0.225% FA)-ACN]; B%: 20%-50%, 20 min) to give the desired compound 2-[2-[2-[2-(2-hydroxyethoxy)ethoxy]ethoxy]ethoxy]ethyl 4-methylbenzenesulfonate (1.8 g, 21% yield) as a yellow oil. 1H NMR (400 MHz, CDCl₃): δ7.82 (d, J=8.4 Hz, 2H), 7.36 (d, J=8.0 Hz, 2H), 4.25-4.14 (m, 2H), 3.72-3.63 (m, 18H), 2.47 (s, 3H).

### 32.2 Preparation of Compound 3

To a solution of 2-[2-[2-[2-(2-hydroxyethoxy)ethoxy]ethoxy]ethoxy]ethyl 4-methylbenzenesulfonate (1.8 g, 4.6 mmol, 1 equiv) and tert-butyl N-tert-butoxy-carbonylcarbamate (996 mg, 4.6 mmol, 1 equiv) in ACN (20 mL) was added K₂CO₃ (1.9 g, 13.8 mmol, 3 equiv). The mixture was stirred at 80° C. for 12 h and quenched by addition of water (30 mL). The mixture was extracted with EtOAc (3×50 mL). The combined organic layer was washed with brine 100 mL, dried over anhydrous sodium sulfate, filtered and concentrated. The residue was purified by prep-HPLC (column: Phenomenex Gemini-NX C18 75*30 mm*3 um; mobile phase: [water(1OmM NH₄HCO₃)-ACN]; B%: 26%-56%, 8 min) to tert-butyl N-tert-butoxycarbonyl-N-[2-[2-[2-[2-(2-hydroxyethoxy)ethoxy]ethoxy]ethoxy]ethyl]carbamate (1.2 g, 59% yield) as a colorless oil. ¹H NMR (400 MHz, CDCl₃): δ3.74-3.60 (m, 20H), 1.55-1.46 (m, 18H).

### 32.3 Preparation of Compound 4

A solution of tert-butyl N-tert-butoxycarbonyl-N-[2-[2-[2-[2-(2-hydroxyethoxy) ethoxy]ethoxy]ethoxy]ethyl]carbamate (4 g, 9.14 mmol, 1 equiv) in DCM (20 mL) and TFA (12 mL) was stirred at 20° C. for 1 h. The reaction mixture was concentrated to give crude 2-[2-[2-[2-(2-aminoethoxy)ethoxy]ethoxy]ethoxy]ethanol (3.2 g, TFA salt) as a yellow oil which was used directly for next step. ¹H NMR (400 MHz, CD₃OD): δ 4.63-4.44 (m, 2H), 3.86-3.64 (m, 16H), 3.20-3.07 (m, 2H).

### 32.4 Preparation of Compound 6

To a solution of 2-[2-[2-[2-(2-aminoethoxy)ethoxy]ethoxy]ethoxy]ethanol (3.2 g, 9.1 mmol, 1 equiv, TFA salt) in THF (20 mL) was added DIEA (3.53 g, 27.3 mmol, 3 equiv) and 4-[(2,2,2-trifluoroacetyl)amino]benzenesulfonyl chloride (2.62 g, 9.1 mmol, 1 equiv). The mixture was stirred at 20° C. for 2 h and concentrated. The residue was purified by column chromatography (SiO₂, Petroleum ether/Ethyl acetate=10:1 to 1:10) to give 2,2,2-trifluoro-N-[4-[2-[2-[2-[2-(2-hydroxyethoxy)ethoxy]ethoxy]ethoxy]ethylsulfamoyl]phenyl]acetamide (2.2 g, 49% yield) as a yellow oil. ¹H NMR (400 MHz, DMSO-d₆): δ9.55 (s, 2H), 7.95-7.88 (m, 2H), 3.61-3.57 (m, 21H).

### 32.5 Preparation of Compound 7

To a solution of 2,2,2-trifluoro-N-[4-[2-[2-[2-[2-(2-hydroxyethoxy)ethoxy]ethoxy]ethoxy]ethylsulfamoyl]phenyl]acetamide (2.2 g, 4.5 mmol, 1 equiv) in THF (10 mL) and H₂O (5 mL) was added KOH (2.53 g, 45 mmol, 10 equiv). The mixture was stirred at 20° C. for 12 h. The organic phase was concentrated in vacuum and then pH value of the aqueous phase was adjusted to about 3 by adding HCl solution (2 M). The mixture was concentrated. The residue was triturated with EtOH (10 mL) at 15° C. for 10 min. The solid was filtered off and the filtrate was concentrated to give the desired compound 4-amino-N-[2-[2-[2-[2-(2-hydroxyethoxy) ethoxy]ethoxy]ethoxy]ethyl]benzenesulfonamide (1.7 g, 96% yield) as a yellow oil. ¹H NMR (400 MHz, DMSO-d₆): δ7.42 (d, J=8.8 Hz, 2H), 6.70-6.55 (m, 2H), 3.67-3.56 (m, 20H).

### 32.6 Preparation of Compound 9

To a solution of 4-amino-N-[2-[2-[2-[2-(2-hydroxyethoxy)ethoxy]ethoxy]ethoxy]ethyl]benzenesulfonamide (600 mg, 1.53 mmol, 1 equiv) and 2-[(5-bromo-2-chloro-pyrimidin-4-yl)amino]-6-fluoro-benzamide (528 mg, 1.53 mmol, 1 equiv) in n-BuOH (10 mL) was added HCl (12 M, 127 μL, 1 equiv). The mixture was stirred at 90° C. for 5 h and concentrated. The residue was purified by prep-HPLC (column: Phenomenex luna C18 150*40 mm* 15 um; mobile phase: [water(0.1%TFA)-ACN]; B%: 24%-54%, 11 min) to give 2-[[5-bromo-2-[4-[2-[2-[2-[2-(2-hydroxyethoxy)ethoxy]ethoxy]ethoxy]ethylsulfamoyl]anilino]pyrimidin-4-yl]amino]-6-fluoro-benzamide (400 mg, 37% yield) as a white solid. ¹H NMR (400 MHz, CD₃OD): δ8.40-8.21 (m, 2H), 7.86-7.69 (m, 4H), 7.55-7.49 (m, 1H), 7.09-7.04 (m, 1H), 3.67-3.48 (m, 18H), 3.10-3.03 (m, 2H).

### 32.7 Preparation of Compound 10

To a solution of 2-[[5-bromo-2-[4-[2-[2-[2-[2-(2-hydroxyethoxy)ethoxy]ethoxy]ethoxy]ethylsulfamoyl]anilino]pyrimidin-4-yl]amino]-6-fluoro-benzamide (100 mg, 142 μmol, 1 equiv) in DMF (1 mL) was added Dess-Martin reagent (120 mg, 285 μmol, 2 equiv). The mixture was stirred at 20° C. for 0.5 h. The reaction mixture was filtered and concentrated under reduced pressure to give the desired compound 2-[[5-bromo-2-[4-[2-[2-[2-[2-(2-oxoethoxy)ethoxy]ethoxy]ethoxy]ethylsulfamoyl]anilino]pyrimidin-4-yl]amino]-6-fluoro-benzamide (99 mg, crude) as a yellow oil which was used directly for next step. LC-MS: MS (ES′): RT=0.822 min, m/z=701.1 [M−H⁺].

### 32.8 Preparation of Compound 13

To a solution of N-(4-fluoro-2-methoxy-5-nitro-phenyl)-4-(1-methylindol-3-yl)pyrimidin-2-amine (8.3 g, 21 mmol, 1 equiv) and tert-butyl N-methyl-N-[2-(methylamino)ethyl]carbamate (4.4 g, 23 mmol, 1.1 equiv) in DMAC (100 mL) was added DIEA (6.8 g, 52.75 mmol, 2.5 equiv). The mixture was stirred at 85° C. for 3 h and diluted with 200 mL water. The suspension was filtered and the filter cake was dried under reduced pressure to give crude compound tert-butyl N-[2-[5-methoxy-N-methyl-4-[[4-(1-methylindol-3-yl)pyrimidin-2-yl]amino]-2-nitro-anilino]ethyl]-N-methyl-carbamate (11 g, crude) as a yellow solid. ¹H NMR (400 MHz, DMSO-d₆): δ8.71 (d, J=14.8 Hz, 1H), 8.41-8.27 (m, 3H), 8.09 (s, 1H), 7.53 (d, J=8.4 Hz, 1H), 7.30-7.11 (m, 3H), 6.93-6.73 (m, 1H), 3.98 (s, 3H), 3.92-3.84 (m, 3H), 3.46-3.38 (m, 2H), 3.34-3.26 (m, 2H), 2.88 (s, 3H), 2.78 (s, 3H), 1.37 (s, 9H).

### 32.9 Preparation of Compound 14

To a solution of tert-butyl N-[2-[5-methoxy-N-methyl-4-[[4-(1-methylindol-3-yl)pyrimidin-2-yl]amino]-2-nitro-anilino]ethyl]-N-methyl-carbamate (5.5 g, 9.8 mmol, 1 equiv) in THF (100 mL) was added Pd/C (1 g, 10% purity, 1.0 equiv) under N₂. The mixture was degassed and purged with H₂ for several times and then stirred under H₂ (15 psi) at 20° C. for 12 h. The mixture was filtered and concentrated to give the desired compound tert-butyl N-[2-[2-amino-5-methoxy-N-methyl-4-[[4-(1-methylindol-3-yl)pyrimidin-2-yl]amino]anilino]ethyl]-N-methyl-carbamate (5 g, crude) as a yellow gum. LC-MS: MS (ES′): m/z=532.3 [M+H⁺].

### 32.10 Preparation of Compound 16

To a solution of tert-butyl N-[2-[2-amino-5-methoxy-N-methyl-4-[[4-(1-methylindol-3-yl)pyrimidin-2-yl]amino]anilino]ethyl]-N-methyl-carbamate (10 g, 18.8 mmol, 1 equiv) in THF (100 mL) was added aq.NaHCO₃ (100 mL, 137 equiv). Then a solution of prop-2-enoyl prop-2-enoate (2.61 g, 20.7 mmol, 1.1 equiv) in THF (10 mL) was added and the mixture was stirred at 25° C. for 12 h. The mixture was quenched by addition water (300 mL) and extracted with EA (3×500 mL). The combined organic layers were washed with brine 300 mL, dried over anhydrous sodium sulfate, filtered and concentrated. The residue was purified by column chromatography (SiO₂, Petroleum ether/Ethyl acetate=10:1 to 1:1) to give the desired compound tert-butyl N-[2-[5-methoxy-N-methyl-4-[[4-(1-methylindol-3-yl)pyrimidin-2-yl]amino]-2-(prop-2-enoylamino)anilino]ethyl]-N-methyl-carbamate (9 g, 81% yield) as a yellow gum. ¹H NMR (400 MHz, CDCl₃): δ 9.74 (s, 1H), 9.05 (s, 1H), 8.91-8.58 (m, 1H), 8.24 (s, 1H), 7.97 (d, J=8.0 Hz, 1H), 7.39-7.29 (m, 1H), 7.27-7.20 (m, 2H), 7.15 (d, J=5.6 Hz, 1H), 6.72 (s, 1H), 6.49-6.24 (m, 2H), 5.78-5.57 (m, 1H), 3.98-3.79 (m, 6H), 3.39-3.19 (m, 2H), 2.92 (s, 2H), 2.78 (s, 3H), 2.62 (s, 3H), 1.39 (s, 9H).

### 32.11 Preparation of Compound 17

A solution of tert-butyl N-[2-[5-methoxy-N-methyl-4-[[4-(1-methylindol-3-yl)pyrimidin-2-yl]amino]-2-(prop-2-enoylamino)anilino]ethyl]-N-methyl-carbamate (2 g, 3.4 mmol, 1 equiv) in DCM (20 mL) and TFA (10 mL) was stirred at 20° C. for 1 h. The reaction mixture was concentrated under reduced pressure to give the desired compound N-[4-methoxy-5-[[4-(1-methylindol-3-yl)pyrimidin-2-yl]amino]-2-[methyl-[2-(methylamino)ethyl]amino]phenyl]prop-2-enamide (2 g, crude, TFA salt) as a white solid. ¹H NMR (400 MHz, CD₃OD): δ8.57 (s, 1H), 8.35 (s, 1H), 8.02 (d, J=6.8 Hz, 2H), 7.64-7.19 (m, 4H), 7.07 (s, 1H), 6.70-6.52 (m, 1H), 6.51-6.36 (m, 1H), 5.88-8.81 (dd, J=1.7, 10.1 Hz, 1H), 3.97 (d, J=9.6 Hz, 6H), 3.50 (t, J=5.6 Hz, 2H), 3.25 (t, J=5.6 Hz, 2H), 2.79 (d, J=10.0 Hz, 6H).

### 32.12 Preparation of Compound I-32

To a solution of N-[4-methoxy-5-[[4-(1-methylindol-3-yl)pyrimidin-2-yl]amino]-2-[methyl-[2-(methylamino)ethyl]amino]phenyl]prop-2-enamide (84 mg, 141 μmol, 1 equiv, TFA salt) in DCM (5 mL) was added TEA (143 mg, 1.42 mmol, 10 equiv) and NaBH(OAc)₃ (299 mg, 1.42 mmol, 10 equiv). Then a solution of 2-[[5-bromo-2-[4-[2-[2-[2-[2-(2-oxoethoxy)ethoxy]ethoxy]ethoxy]ethylsulfamoyl]anilino]-pyrimidin-4-yl]amino]-6-fluoro-benzamide (99 mg, 141 μmol, 1 equiv) in DCM (1 mL) was added. The mixture was stirred at 20° C. for 1 h and concentrated. The residue was purified by prep-HPLC (column: Phenomenex luna C18 150*25 mm* 10 um; mobile phase: [water(0.225% FA)-ACN]; B %: 18%-48%, 10 min) to give desired compound 2-[[5-bromo-2-[4-[2-[2-[2-[2-[2-[2-[5-methoxy-N-methyl-4-[[4-(1-methylindol-3-yl)pyrimidin-2-yl]amino]-2-(prop-2-enoylamino)anilino]ethyl-methyl-amino]ethoxy]-ethoxy]ethoxy]ethoxy]ethylsulfamoyl]anilino]pyrimidin-4-yl]amino]-6-fluoro-benzamide (15 mg, 9% yield, Formate salt) as a yellow solid. ¹H NMR (400 MHz, CD₃OD): δ 9.27-7.72 (m, 6H), 7.68-7.48 (m, 4H), 7.36-7.24 (m, 2H), 7.15-7.03 (m, 3H), 6.88-6.76 (m, 2H), 6.51-6.42 (m, 1H), 6.39-6.29 (m, 1H), 5.72 (d, J=11.2 Hz, 1H), 3.85 (s, 3H), 3.78 (s, 3H), 3.52 (s, 3H), 3.37-3.22 (m, 16H), 3.11 (s, 2H), 2.86 (t, J=5.2 Hz, 4H), 2.60 (s, 3H), 2.50 (s, 2H). LC-MS: MS (ES′): RT=1.973 min, m/z=1170.4 [M+H⁺], LC-MS METHOD 25.

### Example 33—Synthesis of Compound I-33 (synthesized according to General Scheme G in a similar manner to Compound I-32)

2-[[5-bromo-2-[4-[2-[2-[2-[2-[5-methoxy-N-methyl-4-[[4-(1-methylindol-3-yl)pyrimidin-2-yl]amino]-2-(prop-2-enoylamino)anilino]ethyl-methyl-amino]ethoxy]ethoxy]ethyl-sulfamoyl]anilino]pyrimidin-4-yl]amino]-6-fluoro-benzamide (16 mg, 18% yield) as a yellow solid. ¹H NMR (400 MHz, DMSO-d₆) δ10.19 (s, 1H), 10.08 (m, 2H), 9.18 (s, 1H), 8.66 (s, 1H), 8.41-8.28 (m, 3H), 8.23-8.09 (m, 3H), 7.92-7.77 (m, 3H), 7.62 (d, J=8.2 Hz, 2H), 7.55-7.39 (m, 3H), 7.03 (s, 5H), 6.60-6.45 (m, 1H), 6.24 (d, J=17.2 Hz, 1H), 5.78-5.64 (m, 1H), 3.92-3.84 (m, 6H), 3.47 (d, J=4.4 Hz, 8H), 2.86-2.80 (m, 4H), 2.69 (s, 3H), 2.55 (s, 2H), 2.42-2.38 (m, 2H), 2.20 (s, 3H). LC-MS: MS (ES⁺): RT=1.912 min, m/z=1082.3, 1084.3 [M+H⁺], LC-MS METHOD 25.

### Example 34—Synthesis of Compound I-34 (synthesized according to General Scheme G in a similar manner to Compound I-32)

2-[[5-bromo-2-[4-[2-[2-[2-[2-[2-[2-[2-[2-[5-methoxy-N-methyl-4-[[4-(1-methylindol-3-yl)pyrimidin-2-yl]amino]-2-(prop-2-enoylamino)anilino]ethyl-methyl-amino]ethoxy]ethoxy]ethoxy]-ethoxy]ethoxy]ethoxy]ethylsulfam oyl]anilino]pyrimidin-4-yl]amino]-6-fluoro-benzamide (19.65 mg, 11% yield, Formate salt) as a light yellow solid. ¹H NMR (400 MHz, DMSO-d₆): δ10.13 (s, 1H), 10.00-9.80 (m, 2H), 9.17 (s, 1H), 8.69 (s, 1H), 8.41-8.10 (m, 7H), 7.89-7.82 (m, 3H), 7.64 (d, J=8.8 Hz, 2H), 7.55-7.46 (m, 3H), 7.24-7.01 (m, 5H), 6.60-6.53 (m, 1H), 6.26 (d, J=16.0 Hz, 1H), 5.75 (d, J=10.8 Hz, 1H), 3.92 (s, 3H), 3.86 (s, 3H), 3.55-3.39 (m, 28H), 2.86-2.85 (m, 4H), 2.71 (s, 3H), 2.23 (s, 3H). LC-MS: MS (ES⁺): RT=2.009 min, m/z=629.7 [M/2+H⁺], LC-MS METHOD 25.

### Example 35—Synthesis of Compound I-35 (Synthesized according to General Scheme H)

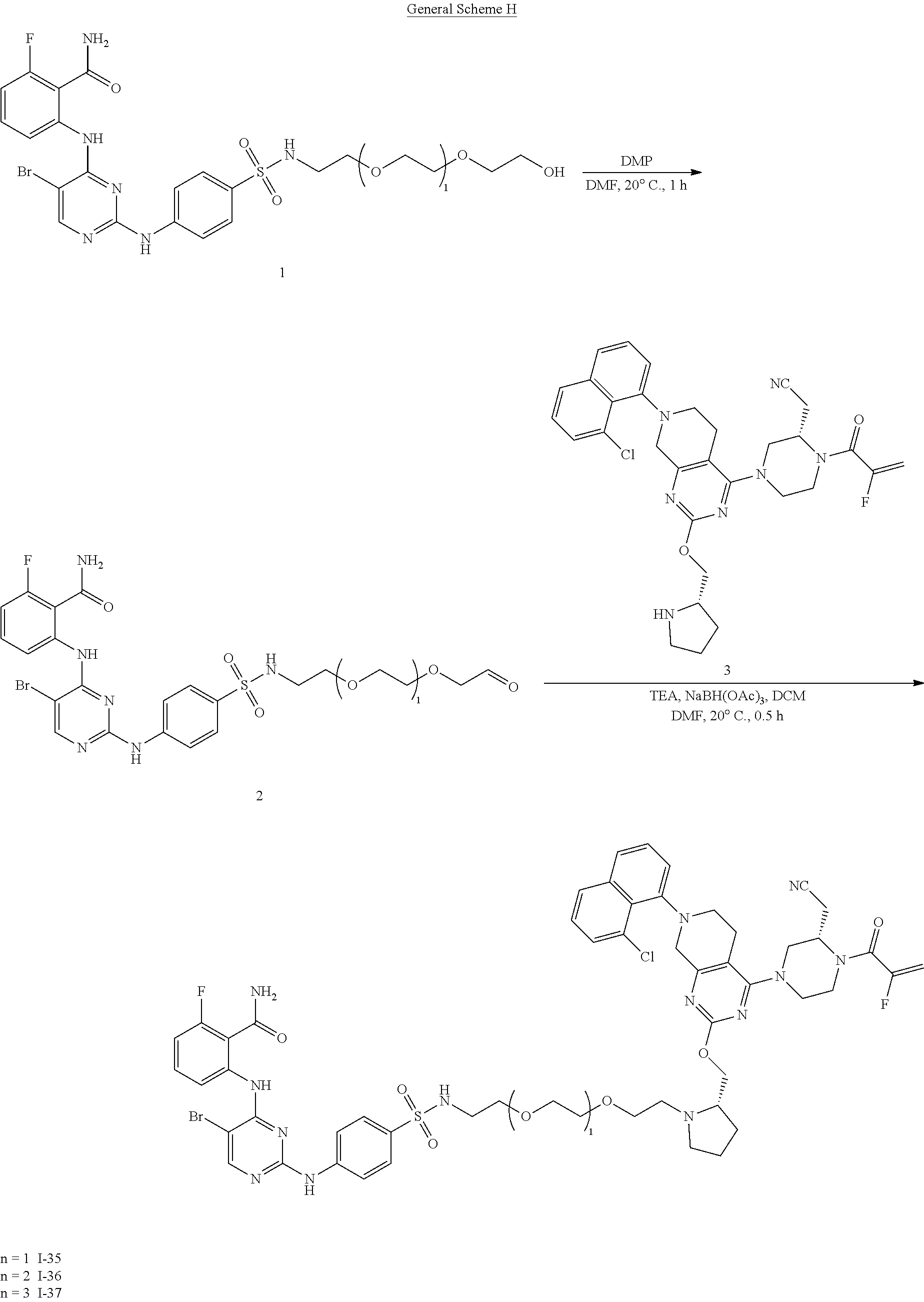

### 35.1 Preparation of Compound 2

To a solution of compound 1 (50 mg, 68 μmol, 1 equiv, TFA salt) in DMF (1 mL) was added DMP (43 mg, 103 μmol, 32 μL, 1.5 equiv). The mixture was stirred at 20° C. for 1 h. The reaction mixture was used for next step without purification. LC-MS: MS (ES⁺): RT=0.800 min, m/z=613.0 [M+H⁺].

### 35.2 Preparation of Compound I-35

To a solution of compound 3 (40 mg, 56 μmol, 1 equiv, TFA salt) in DCM (4 mL) was added TEA (57 mg, 568 μmol, 79 μL, 10 equiv) and NaBH(OAc)₃ (120 mg, 568 μmol, 10 equiv). Then a solution of compound 2 (41 mg, 68 μmol, 1.2 equiv) in DMF (1 mL) was added drop-wise. The mixture was stirred at 20° C. for 0.5 h. The mixture was purified by prep-HPLC (column: Phenomenex Gemini-NX C18 75*30 mm*3 um; mobile phase: [water(10 mM NH4HCO₃)-ACN]; B%: 48%-78%, 8 min) to give the desired product 2-((5-bromo-2-((4-(N-(2-(2-(2-((S)-2-(((7-(8-chloronaphthalen-1-yl)-4-((S)-3-(cyanomethyl)-4-(2-fluoroacryloyl)-piperazin-1-yl)-5,6,7,8-tetrahydropyrido [3,4-d]pyrimidin-2-yl)oxy)methyl)pyrrolidin-1-yl)ethoxy)ethoxy)ethyl)sulfamoyl)phenyl)amino)pyrimidin-4-yl)amino)-6-fluorobenzamide (15 mg, 12 μmol, 97% yield) as an off-white solid. ¹H NMR (400 MHz, CD₃OD) δ8.38-8.31 (m, 1H), 8.22 (d, J=5.3 Hz, 1H), 7.83-7.75 (m, 3H), 7.68 (d, J=8.8 Hz, 2H), 7.65-7.61 (m, 1H), 7.50-7.41 (m, 3H), 7.36-7.30 (m, 1H), 7.28-7.20 (m, 1H), 7.00-6.92 (m, 1H), 5.41-5.21 (m, 2H), 4.41-3.97 (m, 7H), 3.63-3.38 (m, 11H), 3.20-2.86 (m, 11H), 2.69-2.52 (m, 2H), 2.41-2.30 (m, 1H), 2.07-1.93 (m, 1H), 1.84-1.64 (m, 3H). LC-MS: MS (ES⁺): RT=0.732 min, m/z=1186.2 [M+H⁺], LCMS method METHOD 40.

### Example 36—Synthesis of Compound I-36 (synthesized according to General Scheme H in a similar manner to Compound I-35)

2-[[5-bromo-2-[4-[2-[2-[2-[2-[2-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]ethoxy]ethoxy]ethoxy]ethoxy]ethylsulfamoyl]anilino]pyrimidin-4-yl]amino]-6-fluoro-benzamide (18 mg, 14 μmol, 27.62% yield) as a white solid. ¹H NMR (400 MHz, CD₃OD-d₄) 6 8.34 (m, 1H), 8.22 (d, J=2.7 Hz, 1H), 8.18 (s, 1H), 7.86-7.75 (m, 3H), 7.74-7.59 (m, 3H), 7.54-7.41 (m, 3H), 7.38-7.30 (m, 1H), 7.29-7.17 (m, 1H), 7.00-6.92 (m, 1H), 5.42-5.22 (m, 2H), 4.43-3.99 (m, 6H), 3.71-3.49 (m, 15H), 3.48-3.38 (m, 5H), 3.27-2.96 (m, 10H), 2.94-2.51 (m, 4H), 2.12-2.01 (m, 1H), 1.90-1.70 (m, 3H). LC-MS: MS (ES⁺): RT=1.799 min, m/z=1274.4 [M+H⁺], LCMS method LC-MS METHOD 40.

### Example 37—Synthesis of Compound I-37 (synthesized according to General Scheme H in a similar manner to Compoud I-35)

2-[[5-bromo-2-[4-[22[2-[2-[2-[2-[2-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]ethoxy]ethoxy]ethoxy]ethoxy]ethoxy]ethoxy]ethylsulfamoyl]anilino]pyrimidin-4-yl]amino]-6-fluoro-benzamide (11.3 mg, 8.05 μmol, 14.17% yield, 97.03% purity) as a yellow solid. ¹H NMR (400 MHz, CD₃OD) δ8.33 (m, 1H), 8.22 (d, J=2.4 Hz, 1H), 7.80 (m, 3H), 7.80-7.78 (d, J=8.4 Hz, 2H), 7.71-7.69 (d, J=8.0 Hz, 1H), 7.50-7.48 (m, 3H), 7.45 (m, 1H), 7.34 (m, 1H), 6.97 (m, 1H), 5.38-5.28 (m, 2H), 4.35-4.16 (m, 6H), 3.59-3.45 (m, 29H), 3.30-3.03 (m, 10H), 2.61-2.57 (m, 2H), 2.41-2.30 (m, 1H), 2.00-1.80 (m, 1H), 1.78-1.69 (m, 3H). LC

### Example 38—Synthesis of Compound I-38 (Synthesized according to General Scheme J)

The compound had the following MS (ES⁺) data: RT=1.826 min, m/z=1362.4 [M+H⁺]; LC-MS METHOD 40.

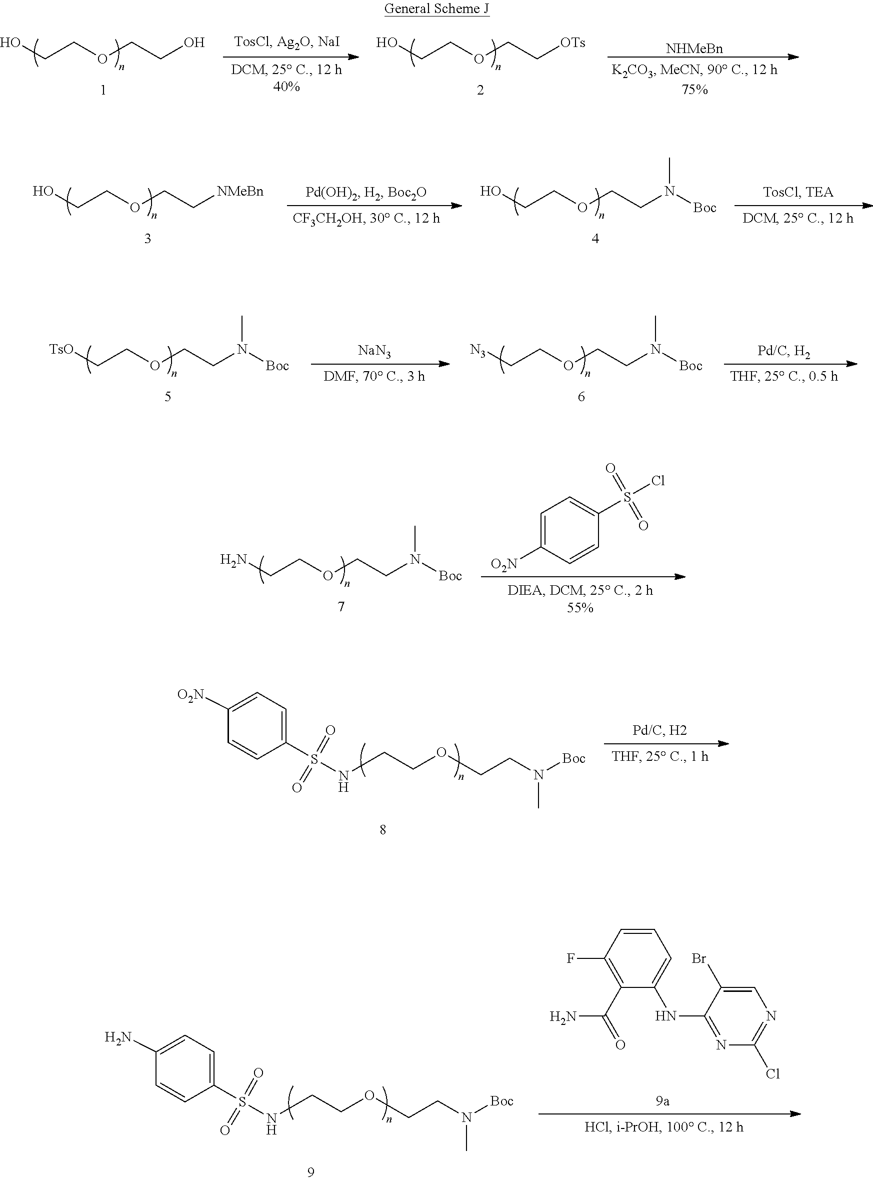

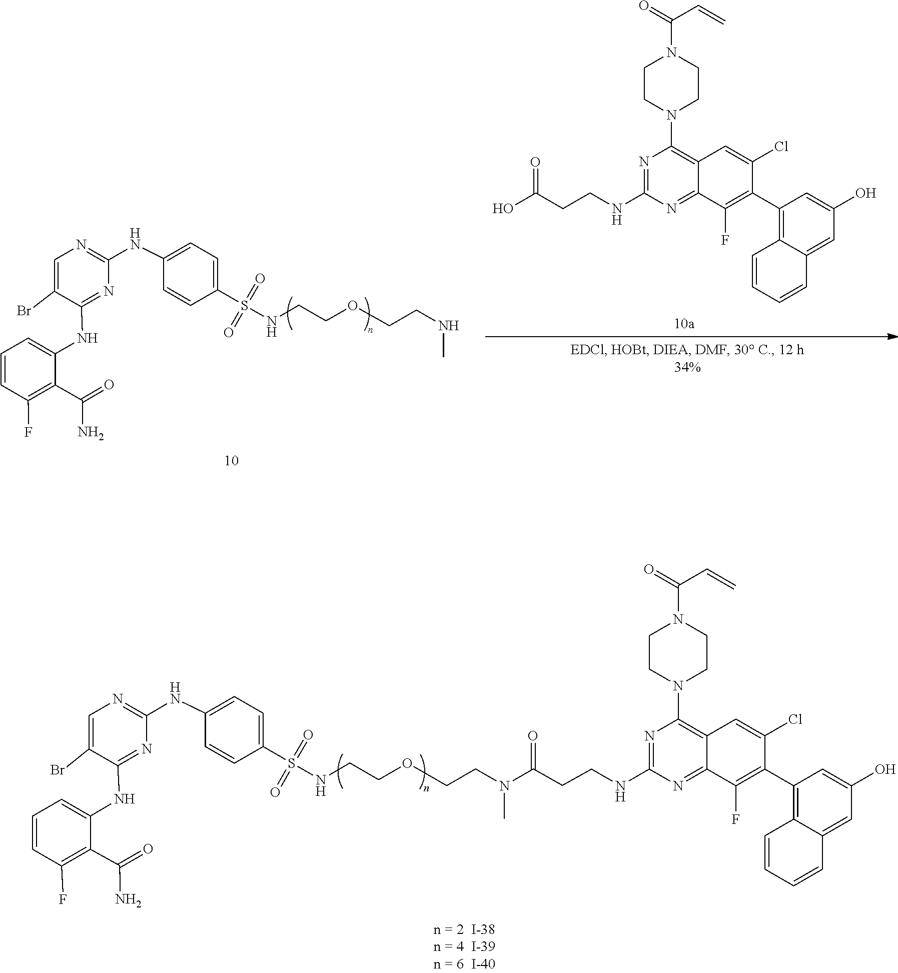

### 38.1 Preparation of Compound 2

To a solution of 2-[2-(2-hydroxyethoxy)ethoxy]ethanol (20 g, 66 mmol, 1.0 equiv) in DCM (200 mL) were added NaI (21 g, 73 mmol, 1.1 equiv), Ag₂0 (46 g, 100 mmol, 1.5 equiv) and 4-methylbenzenesulfonyl chloride (23 g, 60 mmol, 0.9 equiv) at 0° C. The mixture was stirred at 25° C. for 12 h. The solution was filtered. The filtrate was concentrated to give a residue. The residue was purified by silica gel column chromatography (SiO₂, petroleum ether: ethyl acetate=1/1 to 1/2) to give 2-[2-(2-hydroxyethoxy)ethoxy]ethyl 4-methylbenzenesulfonate (16 g, 53 mmol, 40% yield) as a white oil. LC-MS: MS (ES⁺): m/z=305.1 [M+H⁺].

### 38.2 Preparation of Compound 3

To a solution of 2-[2-(2-hydroxyethoxy)ethoxy]ethyl 4-methylbenzenesulfonate (16 g, 53 mmol, 1.0 equiv) and N-methyl-1-phenyl-methanamine (6.4 g, 53 mmol, 1.0 equiv) in MeCN (200 mL) was added K2CO3 (8.7 g, 63 mmol, 1.2 equiv). The mixture was stirred at 90° C. for 12 h. The solution was filtered. The filtrate was concentrated to give a residue. The residue was purified by column chromatography (SiO2, petroleum ether: ethyl acetate=3/1 to 1/2) to give 2-[2-[2-1[benzyl(methyl)amino]ethoxy]ethoxy]ethanol (10 g, 39 mmol, 75% yield) as a white oil. LC-MS: MS (ES⁺): m/z=254.4 [M+H+].

### 38.3 Preparation of Compound 4

To a solution of 2-[2-[2-[benzyl(methyl)amino]ethoxy]ethoxy]ethanol (10 g, 39 mmol, 1.0 equiv) in CF3CH₂OH (100 mL) was added Pd(OH)2/C (1.0 g, 0.7 mmol, 10% purity) and Boc₂O (17 g, 79 mmol, 2.0 equiv) with N₂ for 3 times. The mixture was stirred at 30° C. for 12 h under H2 atmosphere (50 Psi). The reaction mixture was filtered. The filtrate was concentrated under reduced pressure to give tert-butyl-N-[2-[2-(2-hydroxyethoxy)ethoxy]ethyl]-N-methyl-carbamate (8.0 g, 30 mmol) as a white oil. ¹H NMR (400 MHz, CHLOROFORM-d): δ 3.74-3.68 (m, 2H), 3.65-3.55 (m, 8H), 3.40-3.35 (m, 2H), 2.89 (s, 3H), 2.61 (s, 1H), 1.43 (s, 9H). LC-MS: MS (ES⁺): m/z=164.0 [M+H⁺-100].

### 38.4 Preparation of Compound 5

To a solution of tert-butyl N-[2-[2-(2-hydroxyethoxy)ethoxy]ethyl]-N-methyl-carbamate (8.0 g, 30 mmol, 1.0 equiv) and 4-methylbenzenesulfonyl chloride (12 g, 60 mmol, 2.0 equiv) in DCM (150 mL) was added TEA (9.2 g, 91 mmol, 3.0 equiv). The mixture was stirred at 25° C. for 12 h. The solution was concentrated to give a residue. The residue was purified by column chromatography (SiO₂, petroleum ether: ethyl acetate=3/1 to 1/2) to give 2-[2-[2-[tert-butoxycarbonyl(methyl)amino]ethoxy]ethoxy]ethyl-4-methylbenzenesulfonate (6.0 g, 14 mmol) as a white oil. LC-MS: MS (ES⁺): m/z=318.1 [M+H⁺-100].

### 38.5 Preparation of Compound 6

To a solution of 2-[2-[2-[tert-butoxycarbonyl(methyl)amino]ethoxy]ethoxy]ethyl 4-methylbenzenesulfonate (6.0 g, 14 mmol, 1.0 equiv) in DMF (50 mL) was added NaN₃ (2.7 g, 42 mmol, 2.9 equiv). The mixture was stirred at 70° C. for 3 h. To the solution was added water (500 mL). The mixture was extracted with ethyl acetate (2×400 mL). The combined organic phase was washed with brine (400 mL), dried over by anhydrous Na₂SO₄ and filtered. The filtrate was concentrated to give crude tert-butyl-N-1[2-[2-(2-azidoethoxy)ethoxy]ethyl]-N-methyl-carbamate (4.2 g, crude) as a yellow oil. LC-MS: MS (ES⁺): m/z=189.4 [M+H⁺−100].

### 38.6 Preparation of Compound 7

To a solution of tert-butyl N-[2-[2-(2-azidoethoxy)ethoxy]ethyl]-N-methyl-carbamate (1.2 g, 4.2 mmol, 1.0 equiv) in THF (15 mL) was added Pd/C (0.2 g, 10% purity). The mixture solution was stirred under H₂ (15 Psi, balloon) at 25° C. for 0.5 h. The reaction mixture was filtered. The filtrate was concentrated to give crude tert-butyl N-[2-[2-(2-aminoethoxy) ethoxy]ethyl]-N-methyl-carbamate (1.0 g, 3.8 mmol) as a yellow oil.

### 38.7 Preparation of Compound 8

To a solution of tert-butyl N-[2-[2-(2-aminoethoxy)ethoxy]ethyl]-N-methyl-carbamate (1.0 g, 3.8 mmol, 1.0 equiv) in DCM (20 mL) was added 4-nitrobenzenesulfonyl chloride (0.8 g, 3.8 mmol, 1.0 equiv) and DIEA (1.0 g, 7.6 mmol, 1.3 mL, 2.0 equiv). The mixture was stirred at 25° C. for 2 h. The reaction mixture was concentrated under reduced pressure to give a residue. The residue was purified by silica gel column chromatography (SiO₂, petroleum ether: ethyl acetate=3/1 to 1/2) to give tert-butyl-N-methyl-N-[2-[2-[2-[(4-nitrophenyl)sulfonylamino]ethoxy]ethoxy]ethyl]carbamate (0.9 g, 2.1 mmol, 55% yield) as a yellow solid. LC-MS: MS (ES⁺): m/z=348.2 [M+H⁺-100].

### 38.8 Preparation of Compound 9

To a solution of tert-butyl-N-methyl-N-[2-[2-[2-[(4-nitrophenyl)sulfonylamino]ethoxy]ethoxy]ethyl]carbamate (0.5 g, 1.1 mmol, 1.0 equiv) in THF (8 mL) was added Pd/C (0.1 g, 10% purity). The mixture solution was stirred under H₂ (15 Psi, balloon) at 25° C. for 1 h. The reaction mixture was filtered. The filtrate was concentrated to give crude tert-butyl-N-2-[[2-[2-[(4-aminophenyl)sulfonylamino]ethoxy]ethoxy]ethyl]-N-methyl-carbamate (0.4 g, 1.0 mmol) as a light yellow oil.

### 38.9 Preparation of Compound 10

To a solution of tert-butyl-N-[2-[2-[2-[(4-aminophenyl)sulfonylamino]ethoxy]ethoxy]ethyl]-N-methyl-carbamate (0.3 g, 0.7 mmol, 1.0 equiv) and 2-[(5-bromo-2-chloro-pyrimidin-4-yl)amino]-6-fluoro-benzamide (0.2 g, 0.7 mmol, 1.0 equiv) in i-PrOH (3 mL) was added HCl (0.1 M in i-PrOH, 7.2 mL, 1.0 equiv). The mixture was stirred at 100° C. for 12 h. The solution was concentrated to give a residue. The residue was purified by prep-HPLC (column: Phenomenex luna C18 150*40 mm* 15 um; mobile phase: [water(0.1%TFA)-ACN]; B%: 13%-43%, 11 min) to give 2-[[5-bromo-2-[4-[2-[2-[2-(methylamino)ethoxy]ethoxy]ethylsulfamoyl]anilino]pyrimidin-4-yl]amino]-6-fluoro-benzamide (0.1 g, 0.2 mmol, 33% yield, TFA salt) as a white solid. LC-MS: MS (ES⁺): m/z=628.4 [M+H⁺].

### 38.10 Preparation of Compound I-38

To a solution of 2-[[5-bromo-2-[4-[2-[2-[2-(methylamino)ethoxy]ethoxy]ethylsulfamoyl]anilino]pyrimidin-4-yl]amino]-6-fluoro-benzamide (54 mg, 73 μmol, 1.0 equiv, TFA salt) and 3-[[6-chloro-8-fluoro-7-(3-hydroxy-1-naphthyl)-4-(4-prop-2-enoylpiperazin-1-yl)quinazolin-2-yl]amino]propanoic acid (40 mg, 73 μmol, 1.0 equiv) in DMF (2 mL) were added HOBt (20 mg, 145 μmol, 2.0 equiv), EDCI (28 mg, 145 μmol, 2.0 equiv) and DIEA (47 mg, 364 μmol, 63 μL, 5.0 equiv). The mixture was stirred at 30° C. for 12 h. The solution was purified by prep-HPLC (column: Unisil 3-100 C18 Ultra 150*50 mm*3 um; mobile phase: [water(0.225% FA)-ACN]; B%: 25%-55%, 10 min to give 2-[[5-bromo-2-[4-[2-[2-[2-[3-[[6-chloro-8-fluoro-7-(3-hydroxy-1-naphthyl)-4-(4-prop-2-enoylpiperazin-1-yl)quinazolin-2-yl]amino]propanoyl-methyl-amino]ethoxy]ethoxy]ethylsulfamoyl]anilino]pyrimidin-4-yl]amino]-6-fluoro-benzamide (29 mg, 25 μmol, 34% yield, 99% purity) as a white solid. ¹H NMR (400 MHz, METHANOL-d₄): δ8.37-8.24 (m, 2H), 8.19 (d, J=6.2 Hz, 1H,), 7.81-7.74 (m, 3H), 7.74-7.70 (m, 1H), 7.65 (d, J=7.8 Hz, 2H), 7.49-7.41 (m, 1H), 7.38 (t, J=7.2 Hz, 1H,), 7.28-7.21 (m, 2H), 7.20-7.12 (m, 1H), 7.03 (s, 1H), 6.98-6.88 (m, 1H), 6.84-6.71 (m, 1H), 6.26 (d, J=16.4 Hz, 1H), 5.79 (d, J=11.0 Hz, 1H), 3.86 (s, 6H), 3.79-3.60 (m, 4H), 3.59-3.36 (m, 11H), 3.07 (s, 1H), 3.01-2.84 (m, 4H), 2.82-2.69 (m, 2H). LC-MS: MS (ES′): RT=2.882 min, m/z=1157.5 [M+H⁺]; LC-MS METHOD 10.

### Example 39—Synthesis of Compound I-39 (synthesized according to General Scheme J in a similar manner to Compound I-38)

2-[[5-bromo-2-[4-[22[2-[2-[2-[3-[[6-chloro-8-fluoro-7-(3-hydroxy-1-naphthyl)-4-(4-prop-2-enoylpiperazin-1-yl)quina-zolin-2-yl]amino]propanoyl-methyl-amino]ethoxy]ethoxy]ethoxy]ethoxy]ethylsulfamoyl]anilino]pyrimidin-4-yl]amino]-6-fluoro-benzamide (38 mg, 40 μmol, 41% yield, 97% purity) as a white solid. ¹H NMR (400 MHz, METHANOL-d₄): 6 8.38-8.30 (m, 1H), 8.30-8.18 (m, 2H), 7.84-7.76 (m, 3H), 7.75-7.61 (m, 3H), 7.52-7.35 (m, 2H), 7.30-7.21 (m, 2H), 7.21-7.10 (m, 1H), 7.07-7.00 (m, 1H), 6.99-6.89 (m, 1H), 6.86-6.72 (m, 1H), 6.26 (d, J=16.4 Hz, 1H), 5.85-5.72 (m, 1H), 3.94-3.80 (m, 6H), 3.79-3.66 (m, 3H), 3.61-3.36 (m, 20H), 3.09 (s, 1H), 2.99 (s, 2H), 2.90 (s, 2H), 2.81 (d, J=5.8 Hz, 1H), 2.75-2.67 (m, 1H). LC-MS: MS (ES′): RT=2.892 min, m/z=1247.2 [M+H⁺]; LC-MS METHOD 10.

### Example 40—Synthesis of Compound I-40 (synthesized according to General Scheme J in a similar manner to Compound I-38)

2-[[5-bromo-2-[4-[21[2-[2-[2-[2-[12-[3-[[6-chloro-8-fluoro-7-(3-hydroxy-1-naphthyl)-4-(4-prop-2-enoyl-piperazin-1-yl)quinazolin-2-yl]amino]propanoyl-methylamino]ethoxy]ethoxy]ethoxy]ethoxy]ethoxy]-ethoxy]ethylsulfamoyl]anilino]pyrimidin-4-yl]amino]-6-fluoro-benzamide (50 mg, 37 μmol, 51% yield, 100% purity) as an off-white solid. ¹H NMR (400 MHz, DMSO-d₆): δ10.12 (s, 1H), 10.00 (s, 1H), 9.93 (s, 1H), 8.38 (s, 1H), 8.30-8.05 (m, 3H), 7.88-7.72 (m, 4H), 7.63 (d, J=8.8 Hz, 2H), 7.56-7.46 (m, 2H), 7.42 (td, J=4.0, 8.4 Hz, 1H), 7.26 (d, J=2.4 Hz, 1H), 7.22 (d, J=3.8 Hz, 2H), 7.07 (d, J=8.8 Hz, 1H), 7.04 (s, 1H), 6.84 (dd, J=10.4, 16.6 Hz, 1H), 6.17 (dd, J=2.4, 16.6 Hz, 1H), 5.81-5.66 (m, 1H), 3.84-3.66 (m, 7H), 3.53-3.35 (m, 31H), 3.00 (s, 1H), 2.85 (q, J=6.0 Hz, 2H), 2.80 (s, 1H), 2.67-2.56 (m, 2H). LC-MS: MS (ES′): RT=1.600 min, m/z=1357.1 [M+Na⁺]; LC-MS METHOD 40.

### Example 41—Synthesis of Compound I-41 (Synthesized according to General Scheme K)

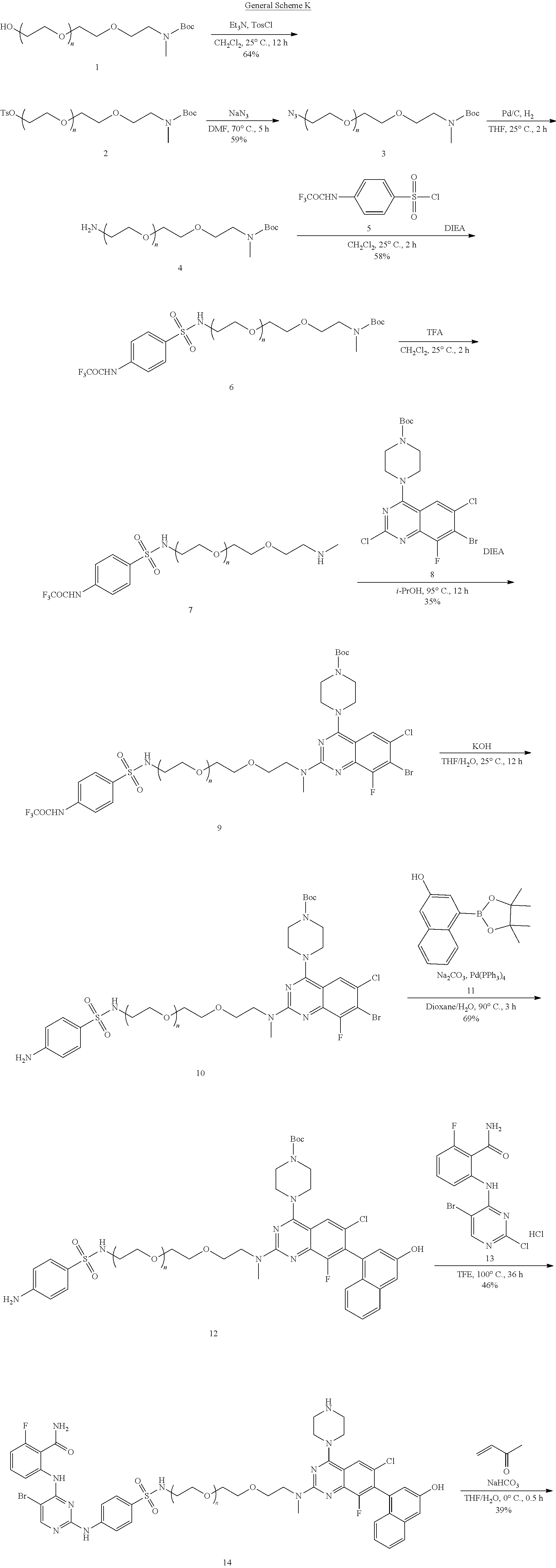

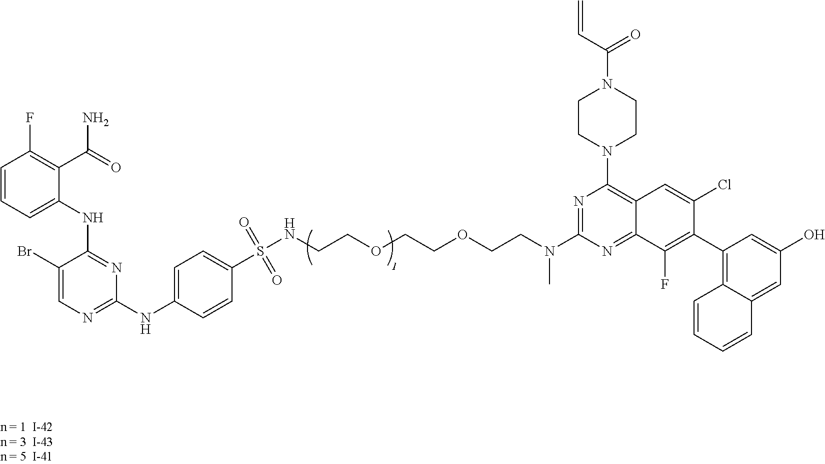

### 41.1 Preparation of Compound 2

To a solution of tert-butyl N-[2-[2-[2-[2-[2-[2-(2-hydroxyethoxy)ethoxy]ethoxy]ethoxy]ethoxy]ethoxy]ethyl]-N-methyl-carbamate (1.5 g, 3.4 mmol, 1.0 equiv) in CH₂Cl₂ (20 mL) was added TosCl (1.30 g, 6.83 mmol, 2.0 equiv) and Et₃N (1.04 g, 10.2 mmol, 1.4 mL, 3.0 equiv), and it was stirred at 25° C. for 12 h. To the reaction mixture was added water (50 mL) and the mixture was extracted with CH₂Cl₂ (50 mL). The combined organic phase was washed with brine (50 mL), dried over anhydrous Na₂SO₄, filtered and concentrated in vacuum. The crude product was purified by silica chromatography (petroleum ether: ethyl acetate=2:1-0:1) to afford 2-[2-[2-[2[2[2-[[2-2[tert-butoxycarbonyl(methyl)amino]ethoxy]ethoxy]ethoxy]ethoxy]ethoxy]ethoxy]ethyl 4-methylbenzenesulfonate (1.3 g, 2.2 mmol, 64% yield) as a light yellow oil. ¹H NMR: (400 MHz, CDCl₃) δ7.81-7.79 (d, J=8.4 Hz, 2H), 7.35-7.33 (d, J=8.0 Hz, 2H), 3.69-3.58 (m, 28H), 2.91 (s, 3H), 2.45 (s, 3H), 1.45 (s, 9H).

### 41.2 Preparation of Compound 3

To a solution of 2-[2-1[2-[2-1[2-[2-[2-1[tert-butoxycarbonyl(methyl)amino]ethoxy]ethoxy]ethoxy]ethoxy]ethoxy]ethoxy]ethyl 4-methylbenzenesulfonate (1.3 g, 2.2 mmol, 1.0 equiv) in DMF (10 mL) was added NaN₃ (1.5 g, 23 mmol, 10.5 equiv), and then it was stirred for 70° C. for 5 hr. The reaction mixture was poured into 50 mL water and extracted with EtOAc (2×20 mL). The organic layers were dried over anhydrous Na₂SO₄, filtered and concentrated to afford crude product. The residue was purified by silica chromatography (PE: EtOAc=2:1-1:1) to afford tert-butyl N-[2-[2-[2-[2-[2-[2-(2-azidoethoxy)ethoxy]ethoxy]ethoxy]ethoxy]ethoxy]ethyl]-N-methyl-carbamate (600 mg, 1.29 mmol, 59% yield) as a colorless oil. ¹H NMR: (400 MHz, CDCl₃) δ3.76-3.51 (m, 24H), 3.45-3.32 (m, 4H), 2.91 (s, 3H), 1.46 (s, 9H).

### 41.3 Preparation of Compound 4

To a solution of tert-butyl N-[2-[2-[2-[2-[2-[2-(2-azidoethoxy)ethoxy]ethoxy]ethoxy]ethoxy]ethoxy]ethyl]-N-methyl-carbamate (600 mg, 1.29 mmol, 1.0 equiv) in THF (20 mL) was added Pd/C (0.2 g, 10% purity), and then it was degassed and purged with H₂. The reaction mixture was stirred at 25° C. for 2 h under 15 psi pressure. The reaction mixture was filtered and the filtrate was concentrated to afford tert-butyl N-[2-[2-[2-[2-[2-[2-(2-aminoethoxy)ethoxy]ethoxy]ethoxy]ethoxy]ethoxy]ethyl]-N-methyl-carbamate (0.57 g, crude) as a colorless oil. ¹H NMR: (400 MHz, CDCl₃) δ3.74-3.49 (m, 28H), 2.91 (s, 3H), 1.46 (s, 9H).

### 41.4 Preparation of Compound 6

To a solution of tert-butyl N-[2-[2-[2-[2-[2-[2-(2-aminoethoxy)ethoxy]ethoxy]ethoxy]ethoxy]ethoxy]ethyl]-N-methyl-carbamate (0.57 g, 1.3 mmol, 1.0 equiv) and DIEA (504 mg, 3.90 mmol, 679 μL, 3.0 equiv) in CH₂Cl₂ (10 mL) was added 4-[(2,2,2-trifluoroacetyl)amino]benzenesulfonyl chloride (449 mg, 1.56 mmol, 1.2 equiv), and then it was stirred at 25° C. for 2 h. The reaction mixture was quenched by 2 mL MeOH, and then it was concentrated to afford crude product. The residue was purified by silica chromatography (PE: EtOAc=1:1-0:1) to afford tert-butyl N-methyl-N-[2-[2-[2-[2-[2-[2-[2-[[4-[(2,2,2-trifluoroacetyl)amino]-phenyl]sulfonylamino]ethoxy]ethoxy]ethoxy]ethoxy]ethoxy]ethoxy]ethyl]carbamate (520 mg, 754 μmol, 58% yield) as a yellow oil. LC-MS: MS (ES⁺): m/z=590.5 [M -99]+. ¹H NMR: (400 MHz, CDCl₃) δ9.45 (m, 1H), 8.00-7.82 (m, 4H), 5.60-5.43 (m, 1H), 3.73-3.30 (m, 26H), 3.18 (q, J=5.3 Hz, 2H), 2.89 (d, J=16.4 Hz, 3H), 1.45 (d, J=4.5 Hz, 9H).

### 41.5 Preparation of Compound 7

To a solution of tert-butyl N-methyl-N-[2-[2-[2-[2-[2-[2-[2-[[4-[(2,2,2-trifluoroacetyl)amino]phenyl]sulfonylamino]ethoxy]ethoxy]ethoxy]ethoxy]ethoxy]ethoxy]ethyl]carbamate (520 mg, 754 mol, 1.0 equiv) in CH₂Cl₂ (3 mL) was added TFA (1 mL), and then it was stirred at 25° C. for 1 h. The reaction mixture was concentrated to afford 2,2,2-trifluoro-N-[4-[2-[2-[2-[2-[2-[2-[2-(methylamino)ethoxy]ethoxy]ethoxy]ethoxy]ethoxy]ethoxy]ethylsulfamoyl]phenyl]acetamide (530 mg, 753 μmol, crude, TFA salt) as a yellow oil and used for the next step directly. LC-MS: MS (ES⁺): m/z=590.5 [M+H⁺].

### 41.6 Preparation of Compound 9

To a solution of 2,2,2-trifluoro-N-[4-[2-[2-[2-[2-[2-[2-[2-(methylamino)ethoxy]ethoxy]ethoxy]-ethoxy]ethoxy]ethoxy]ethylsulfamoyl]phenyl]acetamide (530 mg, 753 μmol, 1.0 equiv, TFA salt) and tert-butyl 4-(7-bromo-2,6-dichloro-8-fluoro-quinazolin-4-yl)piperazine-1-carboxylate (362 mg, 753 μmol, 1.0 equiv) in i-PrOH (8 mL) was added DIEA (487 mg, 3.77 mmol, 656 μL, 5.0 equiv), and then it was stirred at 95° C. for 12 h. The reaction mixture was concentrated to afford crude product. The residue was purified by silica chromatography (PE: EtOAc=1:1-0:1) to afford tert-butyl 4-[7-bromo-6-chloro-8-fluoro-2-[methyl-[2-[2-[2-[2-[2-[2-[2-[[4-[(2,2,2-trifluoroacetyl)amino]phenyl]sulfonylamino]ethoxy]ethoxy]ethoxy]-ethoxy]-ethoxy]ethoxy]ethyl]amino]quinazolin-4-yl]piperazine-1-carboxylate (270 mg, 261 μmol, 35% yield) as a yellow oil. LC-MS: MS (ES⁺): m/z=1034.3 [M+H⁺].

### 41.7 Preparation of Compound 10

To a solution of tert-butyl 4-[7-bromo-6-chloro-8-fluoro-2-[methyl-[2-[2-[2-[2-[2-[2-[2-[[4-[(2,2,2-trifluoroacetyl)amino]phenyl]sulfonylamino]ethoxy]ethoxy]ethoxy]ethoxy]ethoxy]ethoxy]ethyl]amino]quinazolin-4-yl]piperazine-1-carboxylate (270 mg, 261 mol, 1.0 equiv) in THF (1 mL) was added KOH (147 mg, 2.61 mmol, 10.0 equiv) in H₂O (1 mL), and then it was stirred at 25° C. for 12 h. The reaction mixture was added 20 mL water, and then pH was adjusted to 7-8 by 2N HCl at 0° C. After extracted with EtOAc (2×20 mL), the organic layers were dried over anhydrous Na₂SO₄, filtered and concentrated to afford tert-butyl 4-[2-[2-[2-[2-[2-[2-[2-[2-[(4-aminophenyl)sulfonylamino]ethoxy]ethoxy]ethoxy]ethoxy]ethoxy]ethoxy]ethyl-methyl-amino]-7-bromo-6-chloro-8-fluoro-quinazolin-4-yl]piperazine-1-carboxylate (0.25 g, crude) as a yellow oil and used for the next step directly. LC-MS: MS (ES′): m/z=937.6 [M+H⁺].

### 41.8 Preparation of Compound 12

To a solution of tert-butyl 4-[[2[2-[2-[2-[2-[2-[2-[(4-aminophenyl)sulfonylamino]ethoxy]ethoxy]ethoxy]-ethoxy]ethoxy]ethoxy]ethyl-methyl-amino]-7-bromo-6-chloro-8-fluoro-quinazolin-4-yl]piperazine-1-carboxylate (250 mg, 267 μmol, 1.0 equiv) and 4-(4,4,5,5-tetramethyl-1,3,2-dioxaborolan-2-yl)naphthalen-2-ol (144 mg, 533 μmol, 2.0 equiv) in dioxane (2.5 mL) and H₂O (0.5 mL) was added Na₂CO₃ (71 mg, 0.67 mmol, 2.5 equiv) and Pd(PPh₃)₄ (62 mg, 53 μmol, 0.2 equiv), and then it was stirred at 90° C. for 3 h under N₂. The reaction mixture was concentrated to afford crude product. The residue was purified by silica chromatography (PE:EtOAc=1:1-0:1) to afford tert-butyl 4-[2-[2-[2-[2-[2-[2-[2-[2-[(4-aminophenyl)-sulfonylamino]ethoxy]ethoxy]ethoxy]ethoxy]ethoxy]ethoxy]ethyl-methyl-amino]-6-chloro-8-fluoro-7-(3-hydroxy-1-naphthyl)quinazolin-4-yl]piperazine-1-carboxylate (230 mg, 184 μmol, 69% yield) as a yellow oil. LC-MS: MS (ES′): m/z=1000.2 [M+H⁺].

### 41.9 Preparation of Compound 14

To a solution of tert-butyl 4-[2-[2-[2-[2-[2-[2-[2-[2-[(4-aminophenyl) sulfonylamino]ethoxy]ethoxy]-ethoxy]ethoxy]ethoxy]ethoxy]ethyl-methyl-amino]-6-chloro-8-fluoro-7-(3-hydroxy-1-naphthyl)quinazolin-4-yl]piperazine-1-carboxylate (63 mg, 63 μmol, 1.0 equiv) and 2-[(5-bromo-2-chloro-pyrimidin-4-yl)amino]-6-fluoro-benzamide (26 mg, 76 μmol, 1.2 equiv) in TFE (6 mL) was added HCl (12 M, 39 μL, 7.5 equiv), and then it was stirred at 100° C. under MW irradiation for 18 h. LCMS showed -50% of 12 remained, so it was stirred for another 18 h.. The reaction mixture was concentrated to afford crude product. The residue was purified by prep-HPLC (column: 3_Phenomenex Luna C18 75*30 mm*3 um; mobile phase: [water(0.05%HCl)-ACN]; B%: 27%-47%, 6.5 min) to afford 2-[[5-bromo-2-[4-[2-[2-[2-[2-[2-[2-[2-1[[6-chloro-8-fluoro-7-(3-hydroxy-1-naphthyl)-4-piperazin-1-yl-quinazolin-2-yl]-methyl-amino]ethoxy]-ethoxy]ethoxy]ethoxy]ethoxy]ethoxy]ethylsulfamoyl]anilino]pyrimidin-4-yl]amino]-6-fluoro-benzamide (36 mg, 29 μmol, 46% yield, HCl salt) as a yellow solid. LC-MS: MS (ES′): m/z=1210.0 [M+H]+.

### 41.10 Preparation of Compound Compound I-41

To a solution of 2-[[5-bromo-2-[4-[2-[2-[2-[2-[2-[2-[2-[[6-chloro-8-fluoro-7-(3-hydroxy-1-naphthyl)-4-piperazin-1-yl-quinazolin-2-yl]-methyl-amino]ethoxy]ethoxy]ethoxy]ethoxy]ethoxy]ethoxy]-ethylsulfamoyl]anilino]pyrimidin-4-yl]amino]-6-fluoro-benzamide (40 mg, 32 μmol, 1.0 equiv, HCl salt) in THF (2 mL) was added NaHCO₃ (27 mg, 0.32 mmol, 10.0 equiv) in H₂O (1 mL). Prop-2-enoyl chloride (2.9 mg, 32 μmol, 2.6 μL, 1.0 equiv) was added at 0° C. and stirred for 0.5 h. The reaction mixture was poured into 20 mL water, and then it was extracted with EtOAc (2×20 mL). The organic layers were dried over anhydrous Na₂SO₄, filtered and concentrated to afford crude product. The residue was purified by prep-HPLC (column: Phenomenex Gemini-NX C18 75*30 mm*3 um; mobile phase: [water(0.225% FA)-ACN]; B%: 30%-60%, 7 min) to afford 2-[[5-bromo-2-[4-[2-[2-[2-[2-[2-[2-[2-[[6-chloro-8-fluoro-7-(3-hydroxy-1-naphthyl)-4-(4-prop-2-enoylpiperazin-1-yl)quinazolin-2-yl]-methyl-amino]ethoxy]ethoxy]ethoxy]ethoxy]-ethoxy]ethoxy]ethylsulfamoyl]anilino]pyrimidin-4-yl]amino]-6-fluoro-benzamide (16 mg, 13 μmol, 39% yield) as a white solid. LC-MS: MS (ES⁺): RT=2.409 min, m/z=1264.3 [M+H⁺]; LC-MS METHOD 25. ¹H NMR: (400 MHz, CD₃OD) δ8.34 (d, J=8.4 Hz, 1H), 8.21 (s, 1H), 7.81 (d, J=8.8 Hz, 2H), 7.77-7.65 (m, 4H), 7.52-7.43 (m, 1H), 7.42-7.32 (m, 2H), 7.29-7.22 (m, 2H), 7.21-7.14 (m, 1H), 7.03 (d, J=2.3 Hz, 1H), 6.96 (m, 1H), 6.81 (m, 1H), 6.26 (m, 1H), 5.79 (m, 1H), 3.92-3.70 (m, 10H), 3.67-3.36 (m, 22H), 3.24 (s, 3H), 3.04-2.95 (m, 2H).

### Example 42—Synthesis of Compound I-42 (synthesized according to General Scheme K in a similar manner to Compound I-41)

2-[[5-bromo-2-[4-[2-[2-[2-[[6-chloro-8-fluoro-7-(3-hydroxy-1-naphthyl)-4-(4-prop-2-enoylpiperazin-1-yl)quinazolin-2-yl]-methyl-amino]ethoxy]ethoxy]ethylsulfamoyl]-anilino]pyrimidin-4-yl]amino]-6-fluoro-benzamide (23 mg, 21 μmol, 55% yield, 98% purity) as a yellow solid. LC-MS: MS (ES⁺): RT=2.186 min, m/z=1088.2 [M+H⁺], LC-MS METHOD 25. ¹H NMR (400 MHz, CD₃OD) δ8.42-8.27 (m, 2H), 8.19 (s, 1H), 7.84-7.61 (m, 6H), 7.51-7.34 (m, 2H), 7.29-7.21 (m, 2H), 7.19-7.12 (m, 1H), 7.02 (d, J=2.4 Hz, 1H), 6.96-6.88 (m, 1H), 6.84-6.70 (m, 1H), 6.31-6.19 (m, 1H), 5.84-5.74 (m, 1H), 3.87-3.71 (m, 11H), 3.60-3.54 (m, 2H), 3.50-3.44 (m, 2H), 3.42-3.36 (m, 3H), 3.23 (s, 3H), 3.01-2.95 (m, 2H).

### Example 43—Synthesis of Compound I-43 (synthesized according to General Scheme K in a similar manner to I-41)

2-[[5-bromo-2-[4-[1[2[2-[2-[12-[[6-chloro-8-fluoro-7-(3-hydroxy-1-naphthyl)-4-(4-prop-2-enoylpiperazin-1-yl)quinazolin-2-yl]-methyl-amino]ethoxy]ethoxy]ethoxy]ethoxy]ethylsulfamoyl]anilino]pyrimidin-4-yl]amino]-6-fluoro-benzamide (13 mg, 11 μmol, 61% yield, 95.5% purity) as a yellow solid. LC-MS: MS (ES⁺): RT=2.212 min, m/z=1176.2 [M+H⁺], LC-MS METHOD 25; ¹H NMR (400 MHz, CD₃OD) δ8.33 (d, J=9.2 Hz, 1H), 8.20 (s, 1H), 7.79 (d, J=8.8 Hz, 2H), 7.75-7.65 (m, 4H), 7.50-7.36 (m, 2H), 7.28-7.22 (m, 2H), 7.20-7.15 (m, 1H), 7.03 (d, J=2.4 Hz, 1H), 6.98-6.90 (m, 1H), 6.84-6.73 (m, 1H), 6.30-6.20 (m, 1H), 5.83-5.76 (m, 1H), 3.90-3.82 (m, 6H), 3.79-3.71 (m, 6H), 3.62-3.48 (m, 11H), 3.42-3.37 (m, 3H), 3.22 (s, 3H), 2.99 (s, 2H).

### Example 44—Synthesis of Compound I-44

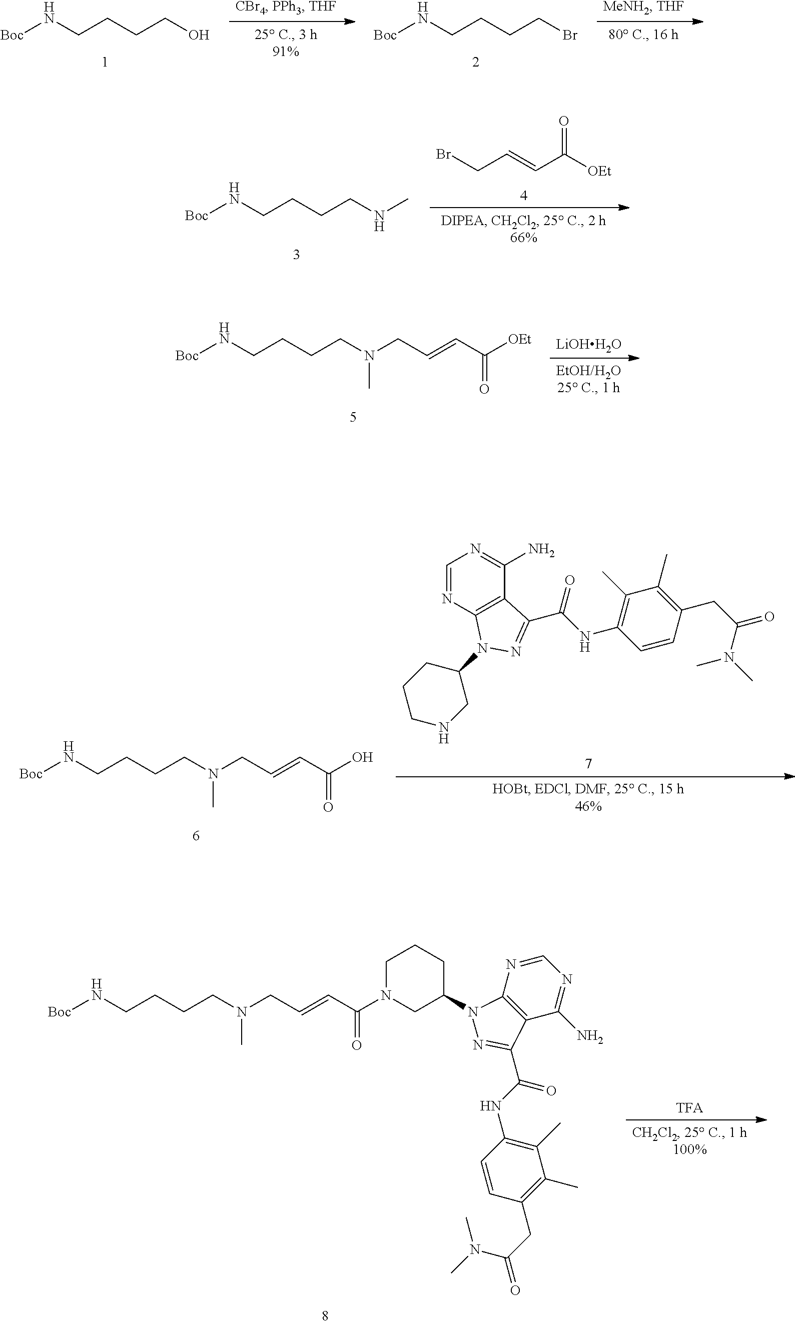

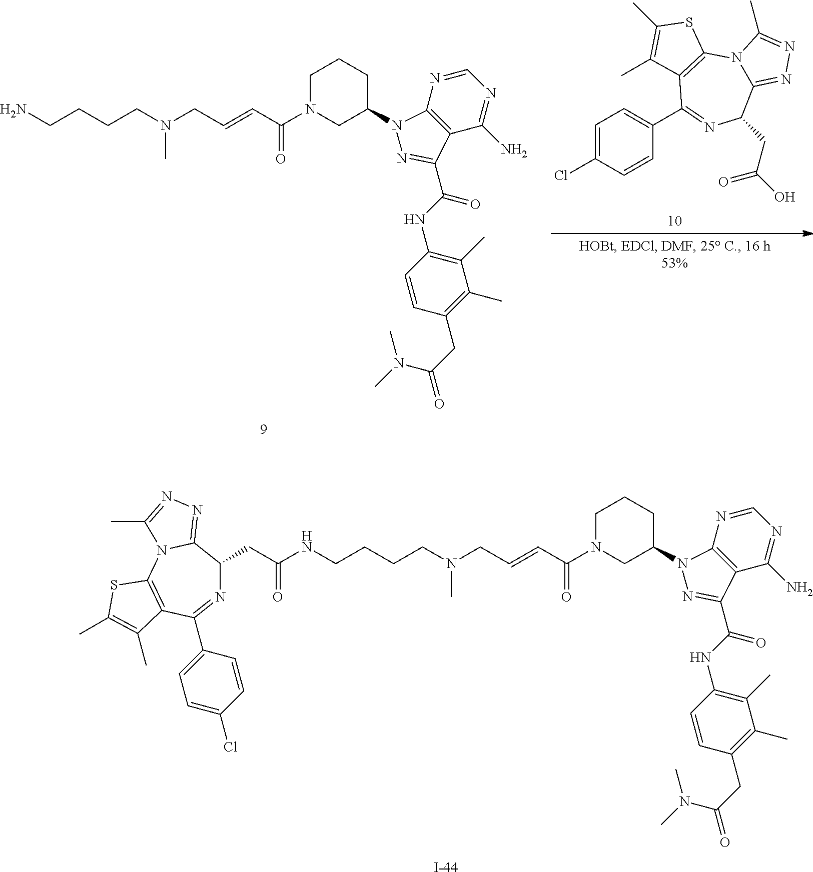

### 44.1 Preparation of Compound 2

To a solution of tert-butyl N-(4-hydroxybutyl)carbamate (9.50 g, 50.2 mmol, 1.0 equiv)) in THF (500 mL) were added PPh₃ (25.0 g, 95.4 mmol, 1.9 equiv) and added CBr₄ (31.6 g, 95.4 mmol, 1.9 equiv) at 25° C. under N₂. The mixture was stirred at 25° C. for 3 h. The reaction mixture was filtered through a pad of celite and the filtrate was concentrated in vacuo. The residue was purified by column chromatography on silica gel (petroleum ether:ethyl acetate=100:1 to 3:1) to afford tert-butyl N-(4-bromobutyl)carbamate (11.5 g, 45.4 mmol, 91% yield) as a yellow oil. ¹H NMR (400 MHz, CDCl₃): δ 4.54 (s, 1H), 3.42 (t, 2H, J=6.8 Hz), 3.15 (q, 2H, J=6.4 Hz), 2.02-1.83 (m, 2H), 1.76-1.72 (m, 2H), 1.44 (s, 9H).

### 44.2 Preparation of Compound 3

A mixture of tert-butyl N-(4-bromobutyl)carbamate (11.5 g, 45.4 mmol, 1.0 equiv) in MeNH₂ (2 M in THF, 227 mL, 10.0 equiv) was stirred at 80° C. for 16 h in a sealed tube. The reaction mixture was diluted with water (100 mL) and the mixture was extracted with ethyl acetate (200 mL*3). The combined organic phase was washed with brine (200 mL), dried with anhydrous Na₂SO₄, filtered and concentrated in vacuo to afford tert-butyl N-[4-(methylamino)butyl]carbamate (7.00 g, 60% purity) as a yellow oil. ¹H NMR (400 MHz, CDCl₃): δ 4.90-4.62 (m, 1H), 3.13 (s, 2H), 2.62 (s, 1H), 2.62-2.60 (m, 1H), 2.44 (s, 2H), 2.50-2.46 (m, 1H), 1.55-1.52 (m, 4H), 1.44 (s, 9H).

### 44.3 Preparation of Compound 5

To a solution of tert-butyl N-[4-(methylamino)butyl]carbamate (1.00 g, 2.97 mmol, 60% purity, 1.0 equiv) and ethyl (E)-4-bromobut-2-enoate (630 mg, 3.26 mmol, 1.1 equiv) in DCM (10 mL) was added DIPEA (767 mg, 5.93 mmol, 1.03 mL, 2.0 equiv) at 25° C. under N₂. The mixture was stirred at 25° C. for 2 h. The reaction mixture was diluted with water (20 mL) and the mixture was extracted with DCM (20 mL*3). The combined organic phase was washed with brine (20 mL), dried with anhydrous Na₂SO₄, filtered and concentrated in vacuo. The residue was purified by column chromatography on silica gel (Dichloromethane:Methanol=100:1 to 5:1) to afford ethyl (E)-4-[4-(tert-butoxycarbonylamino)butyl-methyl-amino]but-2-enoate (614 mg, 1.95 mmol, 66% yield) as a yellow oil. ¹H NMR (400 MHz, CDCl₃): δ 6.98-6.91 (m, 1H), 5.98-5.94 (m, 1H), 4.89 (brs, 1H), 4.20 (q, 2H, J=7.2 Hz), 3.12 (dd, 4H, J=1.2, 6.0 Hz), 2.36 (t, 2H, J=6.8 Hz), 2.21 (s, 3H), 1.53-1.51 (m, 4H), 1.44 (s, 9H), 1.29 (t, 3H, J=7.2 Hz).

### 44.4 Preparation of Compound 6

To a solution of ethyl (E)-4-[4-(tert-butoxycarbonylamino)butyl-methyl-amino]but-2-enoate (300 mg, 954 μmol, 1.0 equiv) in EtOH (3 mL) and H₂O (0.3 mL) was added LiOH.H₂O (120 mg, 2.86 mmol, 3.0 equiv). The mixture was stirred at 25° C. for 1 h. The reaction mixture was concentrated in vacuo and the residue was diluted with H₂O (10 mL). The pH of the mixture was adjusted to 6 by addition of 1 N aq. HCl. The mixture was concentrated in vacuo to afford (E)-4-[4-(tert-butoxycarbonylamino)butyl-methyl-amino]but-2-enoic acid (400 mg, 67% purity) as a yellow oil. ¹H NMR (400 MHz, DMSO-d₆): δ6.72-6.6 (m, 1H), 5.90-5.88 (m, 1H), 3.11-3.08 (m, 2H), 2.91-2.89 (m, 2H), 2.2-2.3 (m, 2H), 2.11 (s, 3H), 1.39-1.35 (m, 4H), 1.36 (s, 9H).

### 44.5 Preparation of Compound 8

To a solution of (E)-4-[4-(tert-butoxycarbonylamino)butyl-methyl-amino]but-2-enoic acid (154 mg, 361 μmol, 67% purity, 2.0 equiv) and 4-amino-N-[4-[2-(dimethylamino)-2-oxo-ethyl]-2,3-dimethyl-phenyl]-1-[(3R)-3-piperidyl]pyrazolo[3,4-d]pyrimidine-3-carboxamide (102 mg, 181 μmol, 1.0 equiv, TFA salt) in DMF (1 mL) were added EDCI (52.0 mg, 271 μmol, 1.5 equiv), HOBt (36.6 mg, 271 μmol, 1.5 equiv) and DIPEA (117 mg, 903 mol, 157 μL, 5.0 equiv) at 25° C. under N₂. The mixture was stirred at 25° C. for 15 h. To the reaction mixture was added water (10 mL) and the mixture was extracted with ethyl acetate (20 mL*3). The combined organic phase was washed with brine (20 mL), dried with anhydrous Na₂SO₄, filtered and concentrated in vacuo. The residue was purified by column chromatography on silica gel (Dichloromethane:Methanol=100:1 to 1:1) to afford tert-butyl N-[4-[[(E)-4-[(3R)-3-[4-amino-3-[[4-[2-(dimethylamino)-2-oxo-ethyl]-2,3-dimethyl-phenyl]carbamoyl]pyrazolo [3,4-d]pyrimidin-1-yl]-1-piperidyl]-4-oxo-but-2-enyl]-methyl-amino]butyl]carbamate (60 mg, 83.5 μmol, 46% yield) as a yellow solid. LC-MS: MS (ES⁺): RT=0.808 min, m/z=719.2 [M+H⁺].

### 44.6 Preparation of Compound 9

A mixture of tert-butyl N-[4-[[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-methyl-amino]butyl]carbamate (50.0 mg, 69.6 μmol, 1.0 equiv) in DCM (1 mL) and TFA (0.5 mL) was stirred at 25° C. or 1 h. The reaction mixture was concentrated in vacuo to afford 4-amino-1-[(3R)-1-[(E)-4-[4-aminobutyl(methyl)amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (58.9 mg, 68.5 μmol, 100% yield, bis-TFA salt) as a yellow oil. LC-MS: MS (ES⁺): RT=0.725 min, m/z=619.6 [M+H⁺].

### 44.7 Preparation of Compound I-44

To a mixture of 2-[(9S)-7-(4-chlorophenyl)-4,5,13-trimethyl-3-thia-1,8,11,12-tetrazatricyclo[8.3.0.02,6]-trideca-2(6), 4,7,10,12-pentaen-9-yl]acetic acid (34.2 mg, 85.3 μmol, 1.0 equiv) and 4-amino-1-[(3R)-1-[(E)-4-[4-aminobutyl(methyl)amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (72.3 mg, 85.3 μmol, 1.0 equiv, 2TFA salt) in DMF (1 mL) were added EDCI (24.5 mg, 128 μmol, 1.5 eq), HOBt (17.3 mg, 128 μmol, 1.5 equiv) and DIPEA (110 mg, 853 μmol, 149 μL, 10.0 equiv) at 25° C. under N₂. The mixture was stirred at 25° C. for 16 h. The mixture was purified by preparative HPLC (column: Phenomenex luna C18 150*25 mm* 10 um; mobile phase: [water(0.225% FA)-ACN]; B%: 18%-48%, 10 min) to afford 4-amino-1-[(3R)-1-[(E)-4-[4-[[2-[(9S)-7-(4-chlorophenyl)-4,5,13-trimethyl-3-thia-1,8,11,12-tetrazatricyclo[8.3.0.02,6]trideca-2(6), 4,7,10,12-pentaen-9-yl]acetyl]amino]butyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (47.5 mg, 45.3 μmol, 53% yield, 100% purity, FA salt) as a white solid. ¹H NMR (400 MHz, DMSO-d₆): δ10.31-10.02 (m, 1H), 8.59 (s, 1H), 8.42-7.83 (m, 4H), 7.54-7.45 (m, 4H), 7.15 (s, 1H), 6.96 (d, 1H, J=8.0 Hz), 6.75-6.43 (m, 2H), 496-4.73 (m, 1H), 4.61-4.23 (m, 2H), 4.10-4.01 (m, 1H), 3.93-3.77 (m, 3H), 3.23-3.01 (m, 1OH), 2.86 (s, 3H), 2.58 (s, 3H), 2.39 (s, 3H), 2.44-2.35 (m, 2H), 2.23-2.11 (m, 1OH), 2.05-2.03 (m, 2H), 1.63-1.57 (m, 4H), 1.53-1.33 (m, 4H). LC-MS: MS (ES⁺): RT=2.322 min, m/z=1001.4 [M+H⁺]; LC-MS method 10

### Example 45—Synthesis of Compound I-45

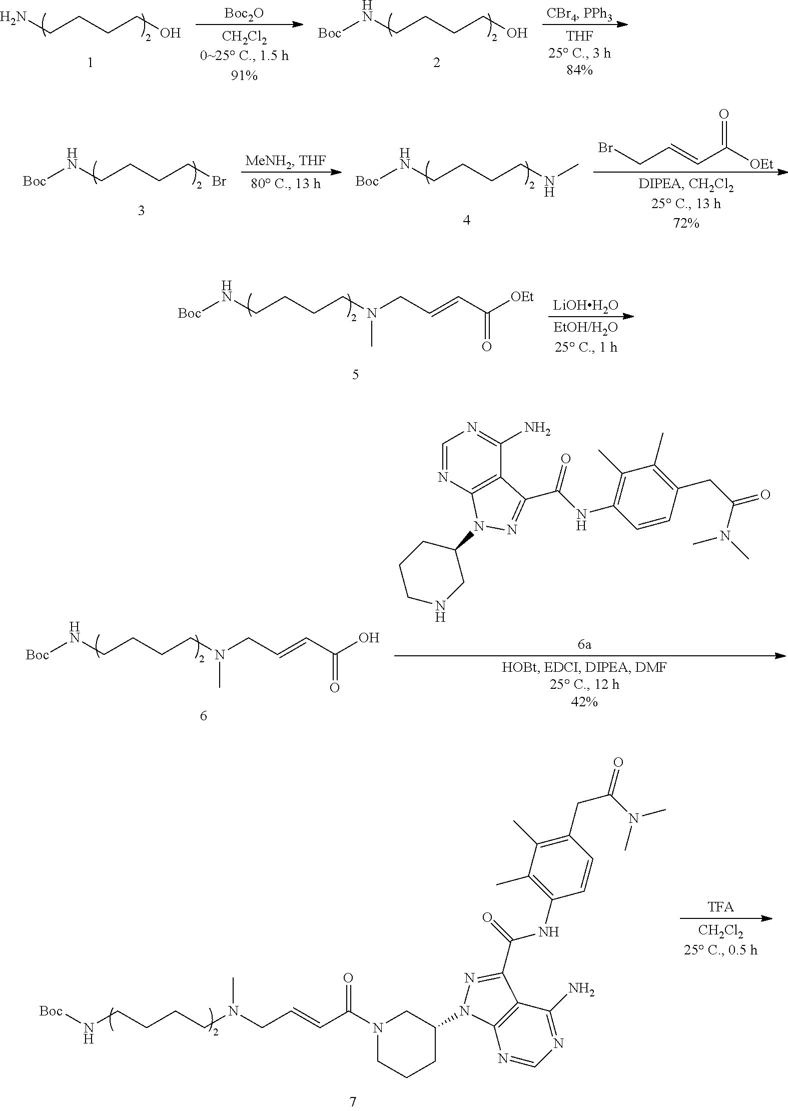

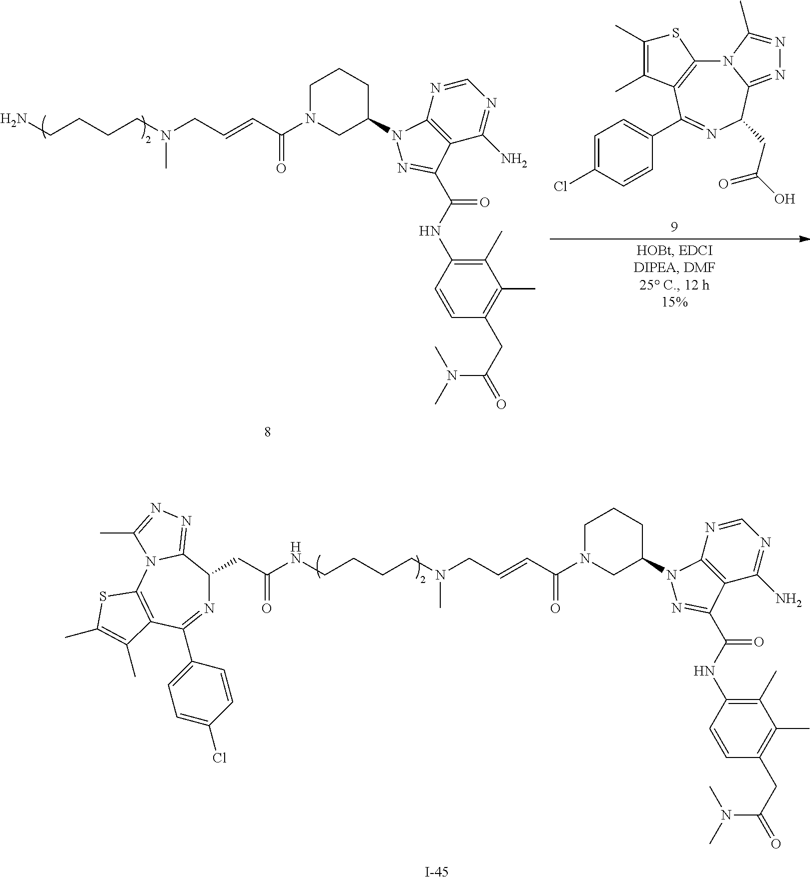

### 45.1 Preparation of Compound 2

To a mixture of 8-aminooctan-1-ol (5.00 g, 27.5 mmol, 1.0 equiv) in CH₂Cl₂ (50 mL) was added Boc₂0 (9.01 g, 33.0 mmol, 9.49 mL, 1.2 equiv) at 0° C. The reaction mixture was stirred at 25° C. for 1.5 h. The reaction mixture was filtered and the filtrate was concentrated in vacuo. The residue was purified by column chromatography on silica gel (Petroleum ether/Ethyl acetate=1/1) to afford tert-butyl N-(8-hydroxyoctyl)carbamate (7.83 g, 25.1 mmol, 91% yield) as a white solid. ¹HNMR (400 MHz, CDCl₃): δ 4.52 (brs, 1H), 3.62 (t, 2H, J=6.4 Hz), 3.15-3.02 (m, 2H), 1.58-1.47 (m, 13H), 1.31-1.23 (m, 9H).

### 45.2 Preparation of Compound 3

Prepared similarly to compound 2 in Compound I-44. The product was purified by column chromatography on silica gel (Petroleum ether/Ethyl acetate=3/1) to afford tert-butyl N-(8-bromooctyl) carbamate (8.28 g, 21.0 mmol, 84% yield) as a colorless oil. ¹ HNMR (400 MHz, CDCl₃): δ 4.53 (brs, 1H), 3.38 (t, 2H, J=6.8 Hz), 3.12-3.03 (m, 2H), 1.87-1.78 (m, 2H), 1.42 (m, 13H), 1.29 (m, 6H).

### 45.3 Preparation of Compound 4

Prepared similarly to compound 3 in Compound I-44 (6.26 g, 24.2 mmol, crude) as a yellow oil. ¹H NMR (400 MHz, CDCl₃): δ3.14-3.01 (m, 2H), 2.52 (t, 2H, J=7.2 Hz), 2.40 (s, 3H), 1.44-1.38 (m, 14H), 1.29-1.25 (m, 9H).

### 45.4 Preparation of Compound 5

Prepared similarly to compound 5 in Compound I-44. The product was purified by column chromatography on silica gel (Dichloromethane/Methanol=10/1) to afford ethyl (E)-4-[8-(tert-butoxycarbonylamino)octyl-methyl-amino]but-2-enoate (6.48 g, 17.5 mmol, 72% yield) as a yellow oil. ¹H NMR (400 MHz, CDCl₃): δ7.00-6.89 (m, 1H), 5.95 (d, 1H, J=15.6 Hz), 4.54 (brs, 1H), 3.13-3.05 (m, 4H), 2.34-2.28 (m, 2H), 2.20 (s, 3H), 1.45-1.39 (m, 13H), 1.32-1.22 (m, 13H).

### 45.5 Preparation of Compound 6

Prepared similarly to compound 6 in Compound I-44 (462 mg, crude) as a yellow oil. ¹H NMR (400 MHz, CDCl₃): δ 6.86-6.62 (m, 1H), 6.08 (d, 1H, J=15.6 Hz), 3.48-3.44 (m, 2H), 3.10-3.04 (m, 2H), 2.80-2.76 (m, 2H), 2.55 (s, 3H), 1.65-1.61 (m, 2H), 1.45-1.39 (m, 15H), 1.31-1.27 (m, 9H).

### 45.6 Preparation of Compound 7

Prepared similarly to compound 8 in Compound I-44. The product was purified by column chromatography on silica gel (Dichloromethane/Methanol=10/1) to afford tert-butyl N-[8-[[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-methyl-amino]octyl]carbamate (220 mg, 0.28 mmol, 42% yield) as a yellow oil. ¹H NMR (400 MHz, CDCl₃): δ8.86 (s, 1H), 8.34 (s, 1H), 7.60-7.48 (m, 1H), 7.04 (d, 1H, J=8.4 Hz), 6.90-6.40 (m, 2H), 6.09-5.80 (m, 1H), 4.97-4.84 (m, 1H), 4.70-4.53 (m, 1H), 4.35-4.10 (m, 1H), 3.80-3.61 (m, 3H), 3.43-3.28 (m, 2H), 3.15-2.94 (m, 9H), 2.75-2.63 (m, 1H), 2.59-2.41 (m, 3H), 2.38-2.17 (m, 1OH), 1.52-1.35 (m, 13H), 1.34-1.07 (m, 11H).

### 45.7 Preparation of Compound 8

Prepared similarly to compound 9 in Compound I-44 to afford 4-amino-1-[(3R)-1-[(E)-4-[8-aminooctyl(methyl)amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (256 mg, crude, 2TFA salt) as a yellow oil. LC-MS: MS (ES⁺): RT=0.680 min, m/z=675.3 [M+H⁺].

### 45.8 Preparation of Compound I-45

To a mixture of 4-amino-[(3R)-1-[(E)-4-[8-aminooctyl(methyl)amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (128 mg, 0.14 mmol, 1.1 equiv, 2TFA salt) and 2-[(9S)-7-(4-chlorophenyl)-4,5,13-trimethyl-3-thia-1,8,11,12-tetrazatricyclo[8.3.0.02,6]trideca-2(6), 4,7,10,12-pentaen-9-yl]acetic acid (52 mg, 0.13 mmol, 1.0 equiv) in DMF (1 mL) were added HOBt (26.3 mg, 0.19 mmol, 1.5 equiv), DIPEA (83.8 mg, 0.65 mmol, 113 μL, 5.0 equiv) and EDCI (37.3 mg, 0.19 mmol, 1.5 equiv) at 25° C. The reaction mixture was stirred at 25° C. for 12 h. The reaction mixture was diluted with water (5 mL) and the mixture was extracted with DCM/MeOH (10/1, 3 mL*3). The combined organic phase was washed with brine (5 mL), dried with anhydrous Na₂SO₄, filtered and concentrated in vacuo. The residue was purified by prep-HPLC (column: Shim-pack C18 150*25*10 um; mobile phase: [water(0.225% FA)-ACN]; B%: 20%-50%, 10 min) to afford 4-amino-[(3R)-1-[(E)-4-[8-[[2-[(9S)-7-(4-chlorophenyl)-4,5,13-trimethyl-3-thia-1,8,11,12-tetrazatricyclo[8.3.0.02,6]trideca-2(6), 4,7,10,12-pentaen-9-yl]acetyl]amino]octyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (20.7 mg, 0.02 mmol, 15% yield, 99% purity) as an off-white solid. ¹H NMR (400 MHz, DMSO-d₆): δ10.23-10.00 (m, 1H), 8.59 (brs, 1H), 8.33 (s, 1H), 8.25 (s, 1H), 8.20-8.00 (m, 2H), 7.56-7.33 (m, 4H), 7.20-7.10 (m, 1H), 6.95 (d, 1H, J=8.0 Hz), 6.66-6.62 (m, 2H), 4.87-4.65 (m, 1H), 4.58-4.15 (m, 2H), 4.12-4.00 (m, 1H), 3.73-3.68 (m, 3H), 3.23-2.96 (m, 10H), 2.86 (s, 3H), 2.58 (s, 3H), 2.40 (s, 3H), 2.36-2.24 (m, 2H), 2.22-2.06 (m, 10H), 2.04-2.00 (m, 2H), 1.63-1.59 (m, 4H), 1.45-1.11 (m, 12H). LC-MS: MS (ES⁺): RT=2.068 min, m/z=1057.5 [M+H⁺]; LC-MS method 25.

### Example 46—Synthesis of Compound I-46

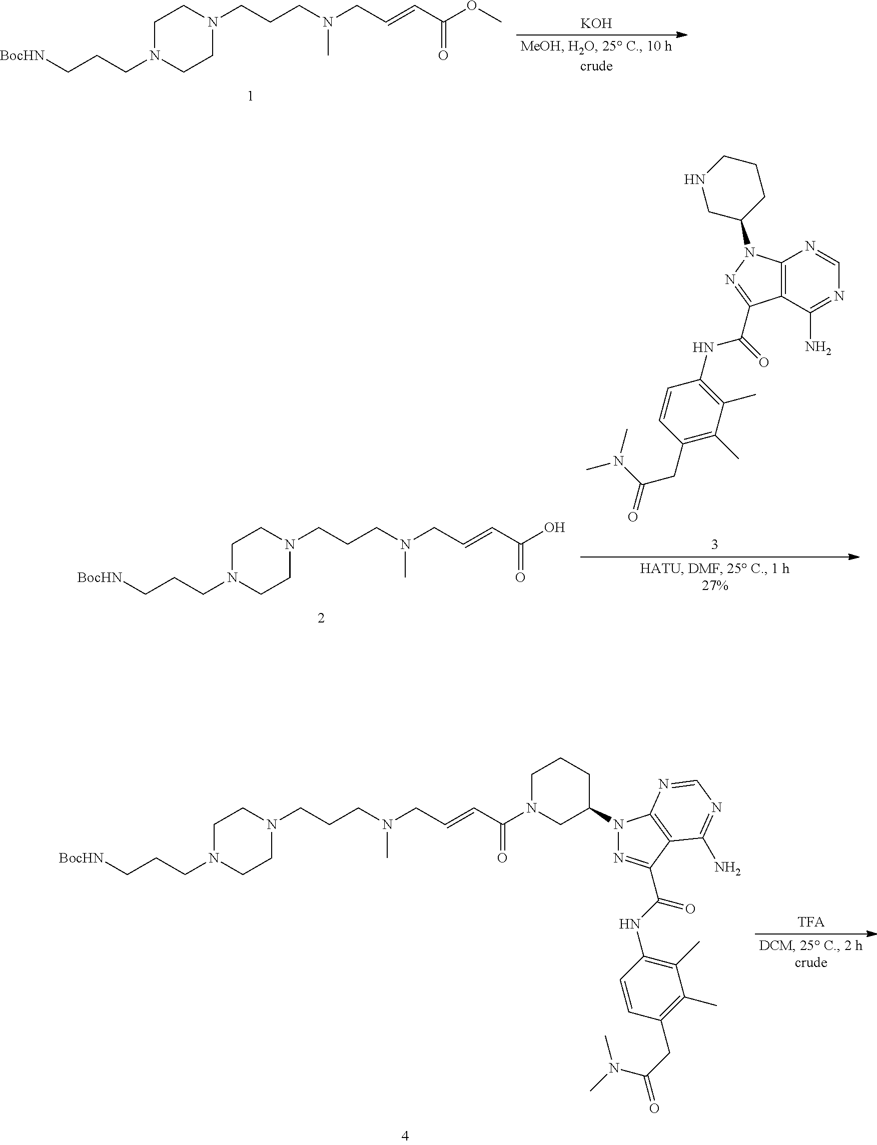 [SMILES: COC(=O)/C=C/CN(C)CCCN1CCN(CCCNC(=O)OC(C)(C)C)CC1 ; "KOH" ; Cc1c(CC(=O)N(C)C)ccc(NC(=O)c2nn([C@@H]3CCCNC3)c3ncnc(N)c23)c1C ; CN(C/C=C/C(=O)O)CCCN1CCN(CCCNC(=O)OC(C)(C)C)CC1 ; Cc1c(CC(=O)N(C)C)ccc(NC(=O)c2nn([C@@H]3CCCN(C(=O)/C=C/CN(C)CCCN4CCN(CCCNC(=O)OC(C)(C)C)CC4)C3)c3ncnc(N)c23)c1C]

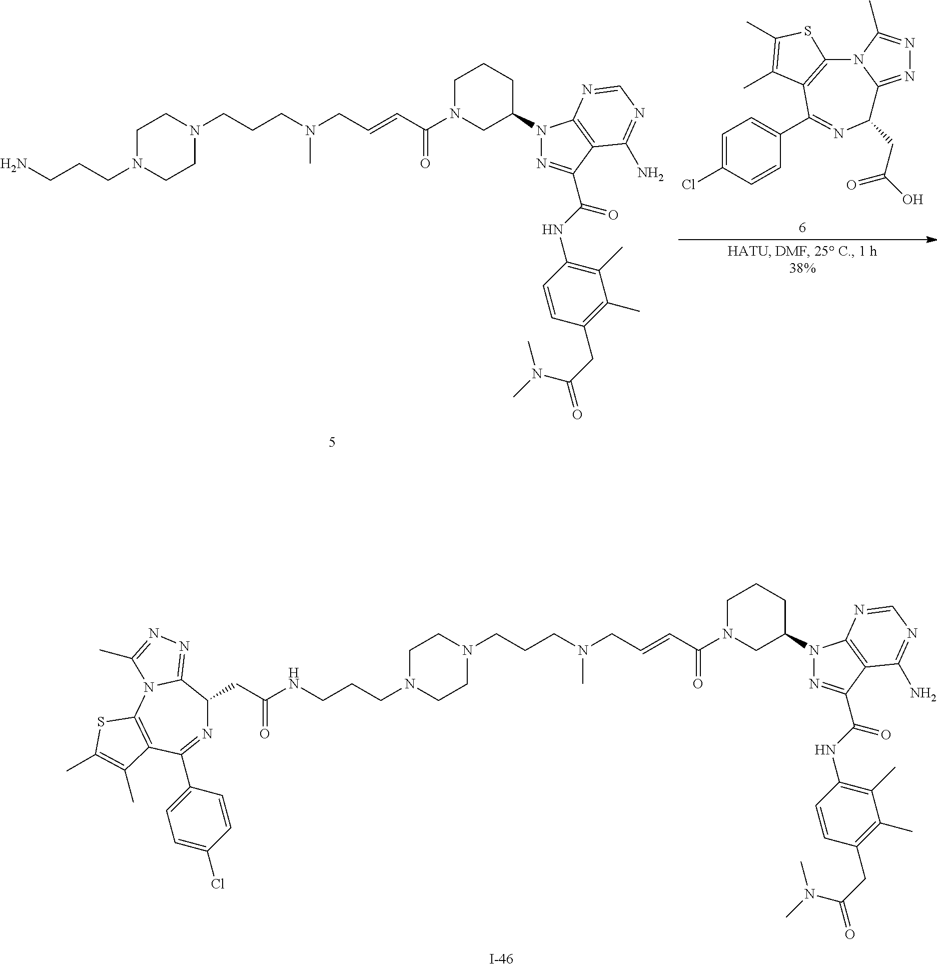

### 46.1 Preparation of Compound 2

To a solution of methyl-(E)-4-[3-[4-[3-(tert-butoxycarbonylamino)propyl]piperazin-1-yl]propyl-methyl-amino]but-2-enoate (200 mg, 432 μmol, 89% purity, 1.0 equiv) in MeOH (2 mL) and H₂O (0.5 mL) was added KOH (72 mg, 1.29 mmol, 3.0 equiv). The mixture was stirred at 25° C. for 10 h. The mixture was concentrated to give crude (E)-4-[3-[4-[3-(tert-butoxycarbonylamino)propyl]piperazin-1-yl]propyl-methyl-amino]but-2-enoic acid (171 mg, crude) as a light yellow solid. LC-MS: MS (ES⁺): RT=0.720 min, m/z=399.3 [M+H⁺].

### 46.2 Preparation of Compound 4

To a solution of (E)-4-[3-[4-[3-(tert-butoxycarbonylamino)propyl]piperazin-1-yl]-propyl-methyl-amino]but-2-enoic acid (171 mg, 429 μmol, 1.0 equiv) and 4-amino-N-[4-[2-(dimethylamino)-2-oxo-ethyl]-2,3-dimethyl-phenyl]-1-[(3R)-3-piperidyl]pyrazolo[3,4-d]pyrimidine-3-carboxamide (246 mg, 436 μmol, 1.0 equiv, TFA salt) in DMF (2 mL) were added DIEA (222 mg, 1.72 mmol, 298.9 μL, 4.0 equiv) and HATU (196 mg, 515 μmol, 1.2 equiv). The mixture was stirred at 25° C. for 1 h. The mixture was purified by prep-HPLC (column: Waters Xbridge 150*25 mm* Sum; mobile phase: [water(10 mM NH4HCO₃)-ACN]; B%: 26%-50%, 7 min) and prep-HPLC (column: Waters Xbridge 150*25 mm* Sum; mobile phase: [water (0.05% ammonia hydroxide v/v)-ACN]; B%: 20%-50%, 10 min) to give tert-butyl-N-[3-[4-[3-[[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-methyl-amino]propyl]piperazin-1-yl]propyl]carbamate (100 mg, 118 μmol, 27% yield, 98% purity) as a white solid. LC-MS: MS (ES⁺): RT=0.917 min, m/z=831.5 [M+H⁺].

### 46.3 Preparation of Compound 5

Prepared similarly to procedures described herein above. The mixture was concentrated to give 4-amino-1-[(3R)-1-[(E)-4-[3-[4-(3-aminopropyl)piperazin-1-yl]propyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (101 mg, crude, TFA salt) as a yellow gum. LC-MS: MS (ES⁺): RT=0.569 min, m/z=731.4 [M+H⁺].

### 46.4 Preparation of Compound I-46

To a solution of 4-amino-1-[(3R)-1-[(E)-4-[3-[4-(3-aminopropyl)piperazin-1-yl]propyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (101 mg, 120 μmol, 1.0 equiv, TFA salt) and 2-[(9S)-7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetic acid (48 mg, 0.12 mmol, 1.0 equiv) in DMF (2 mL) was added DIEA (77 mg, 0.60 mmol, 5.0 equiv) and HATU (54 mg, 0.14 mmol, 1.2 equiv). The mixture was stirred at 25° C. for 1 h. The mixture was purified by prep-HPLC (column: Phenomenex Gemini-NX C18 75*30 mm*3 um; mobile phase: [water(1OmM NH₄HCO₃)-ACN]; B%: 28%-58%, 8 min) to give 4-amino-1-[(3R)-1-[(E)-4-[3-[4-[3-[ [2-[(9S)-7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetyl]amino]propyl]piperazin-1-yl]propyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (50 mg, 45 μmol, 38% yield, 100% purity) as a white solid. ¹H NMR (400 MHz, MeOD): δ8.24 (d, J=10.8 Hz, 1H), 7.46-7.31 (m, 5H), 7.01 (d, J=8.0 Hz, 1H), 6.84-6.37 (m, 2H), 5.04-4.88 (m, 1H), 4.74-3.89 (m, 4H), 3.80 (s, 2H), 3.73-3.47 (m, 1H), 3.46-3.33 (m, 2H), 3.29-3.18 (m, 2H), 3.11 (s, 3H), 3.08-3.02 (m, 1H), 2.99 (s, 3H), 2.69 (s, 3H), 2.66-1.98 (m, 30H), 1.81-1.67 (m, 7H), 1.65-1.58 (m, 1H). LC-MS: MS (ES⁺): RT=2.171 min, m/z=1113.5 [M+H⁺]; LC-MS method 10.

### Example 47—Synthesis of Compound I-47

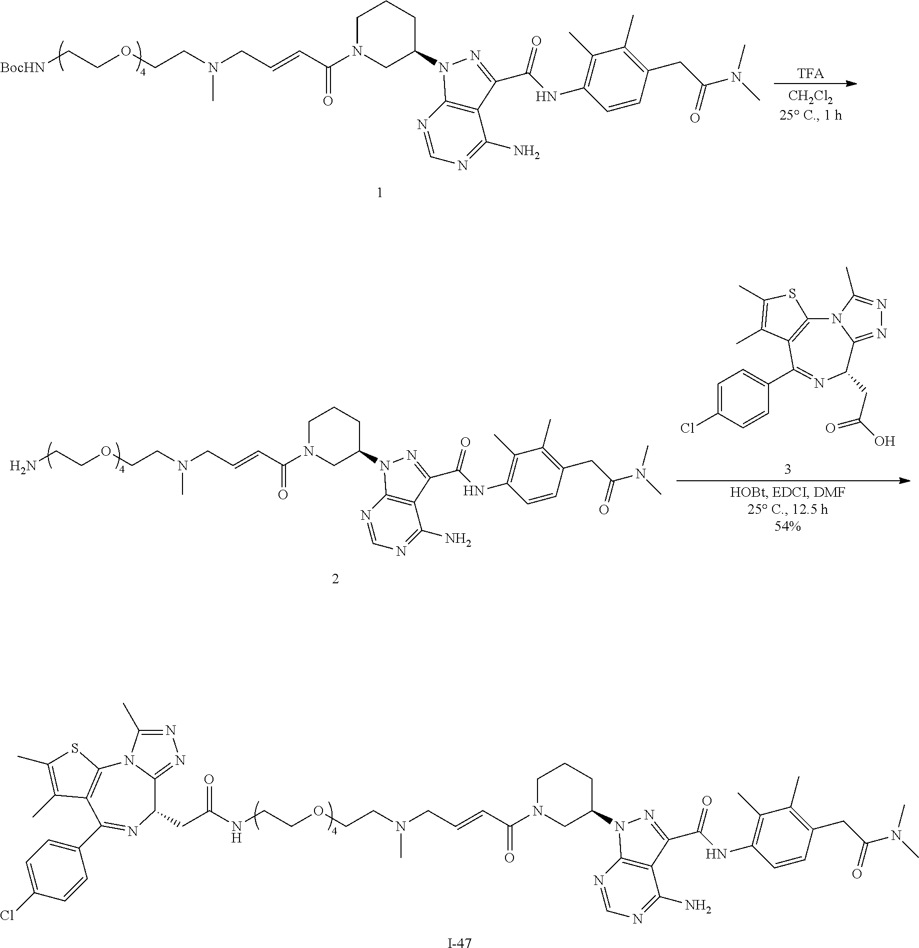

### 47.1 Preparation of Compound 2

Prepared similarly to compound 9 in Compound I-73. The reaction mixture was concentrated in vacuo to afford 4-amino-[(3R)-1-[(E)-4-[2-[2-[2-[2-(2-aminoethoxy) ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (118 mg, 134 mol, 99% yield, TFA) as a yellow solid. LC-MS: MS (ES⁺): RT=0.443 min, m/z=767.3 [M+H⁺].

### 47.2 Preparation of Compound I-47

A mixture of 2-[(9S)-7-(4-chlorophenyl)-4,5,13-trimethyl-3-thia-1,8,11,12-tetrazatricyclo[8.3.0.02,6]trideca-2(6), 4,7,10,12-pentaen-9-yl]acetic acid (45.5 mg, 114 μmol, 1.0 equiv) in DMF (0.2 mL) was added DIPEA (73.4 mg, 568 μmol, 98.9 μL, 5.0 equiv), EDCI (32.6 mg, 170 μmol, 1.5 equiv) and HOBt (23.0 mg, 170 μmol, 1.5 equiv) was stirred at 25° C. for 0.5 h. To the mixture was added 4-amino-1-[(3R)-1-[(E)-4-[2-[2-[2-[2-(2-aminoethoxy) ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (100 mg, 113.51 mol, 1.0 equiv, TFA). The mixture was stirred at 25° C. for 12 h. The reaction mixture was diluted with water (20 mL) and extracted with EtOAc (20 mL*3). The combined organic phase was washed with brine (20 mL), dried over anhydrous Na₂SO₄, filtered and concentrated in vacuo. The residue was purified by prep-HPLC (column: Phenomenex luna C18 150*25 mm* 10 um; mobile phase: [water(0.225% FA)-ACN]; B%: 23%-43%, 10 min) to afford 4-amino-1-[(3R)-1-[(E) -4-[21[2-[2-[2-[[2-[(9S)-7-(4-chlorophenyl)-4,5,13-trimethyl-3-thia-1,8,11,12-tetrazatricyclo [8.3.0.02,6]trideca-2(6), 4,7,10,12-pentaen-9-yl]acetyl]amino]ethoxy]ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (70 mg, 60.9 μmol, 54% yield, 100% purity) as a yellow solid. ¹HNMR (400 MHz, MeOD-d₄): S 8.37 (brs, 1H), 8.27-8.18 (m, 1H), 7.48-7.29 (m, 5H), 7.04-6.59 (m, 3H), 5.01-4.91 (m, 1H), 4.70-4.04 (m, 3H), 4.04-3.55 (m, 22H), 3.49-3.38 (m, 4H), 3.30-3.20 (m, 2H), 3.19-3.11 (m, 4H), 3.00 (s, 3H), 2.82-2.72 (m, 3H), 2.71-2.64 (m, 3H), 2.48-2.33 (m, 4H), 2.31-2.20 (m, 7H), 2.02-1.99 (m, 1H), 1.70-1.66 (m, 4H). The signal of the NH₂ proton was not observed. LC-MS: MS (ES⁺): RT=2.353 min, m/z=1151.5 [M+H⁺]; LC-MS method 10.

### Example 48—Synthesis of Compound I-48

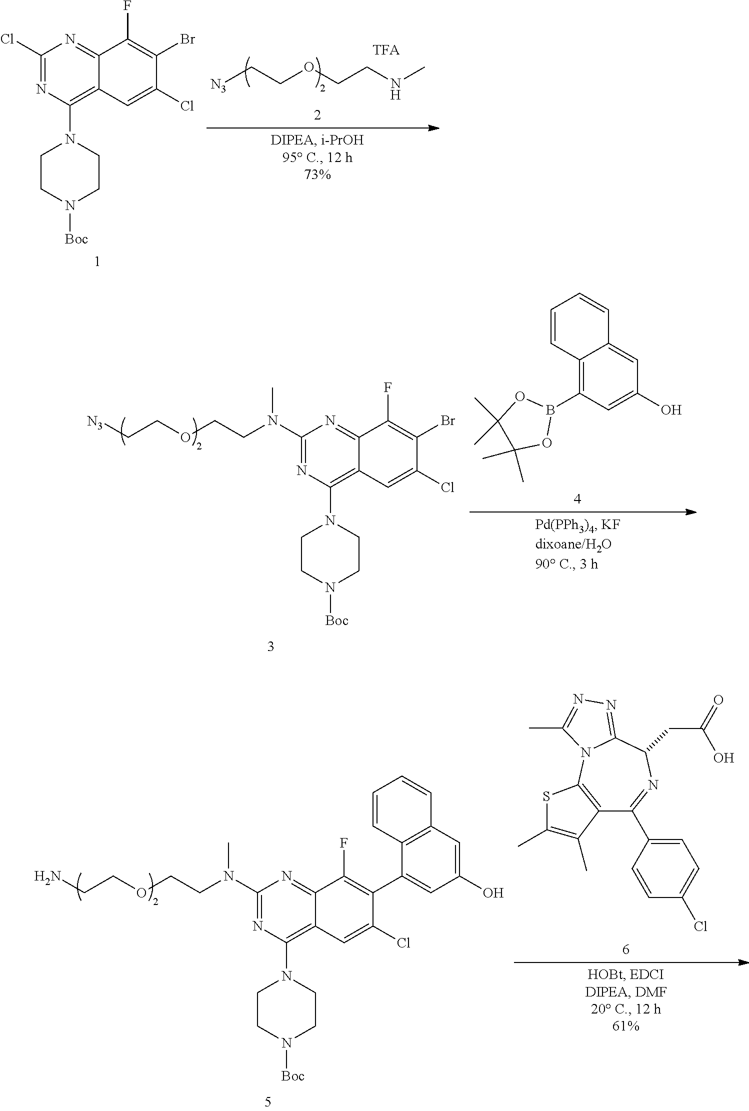

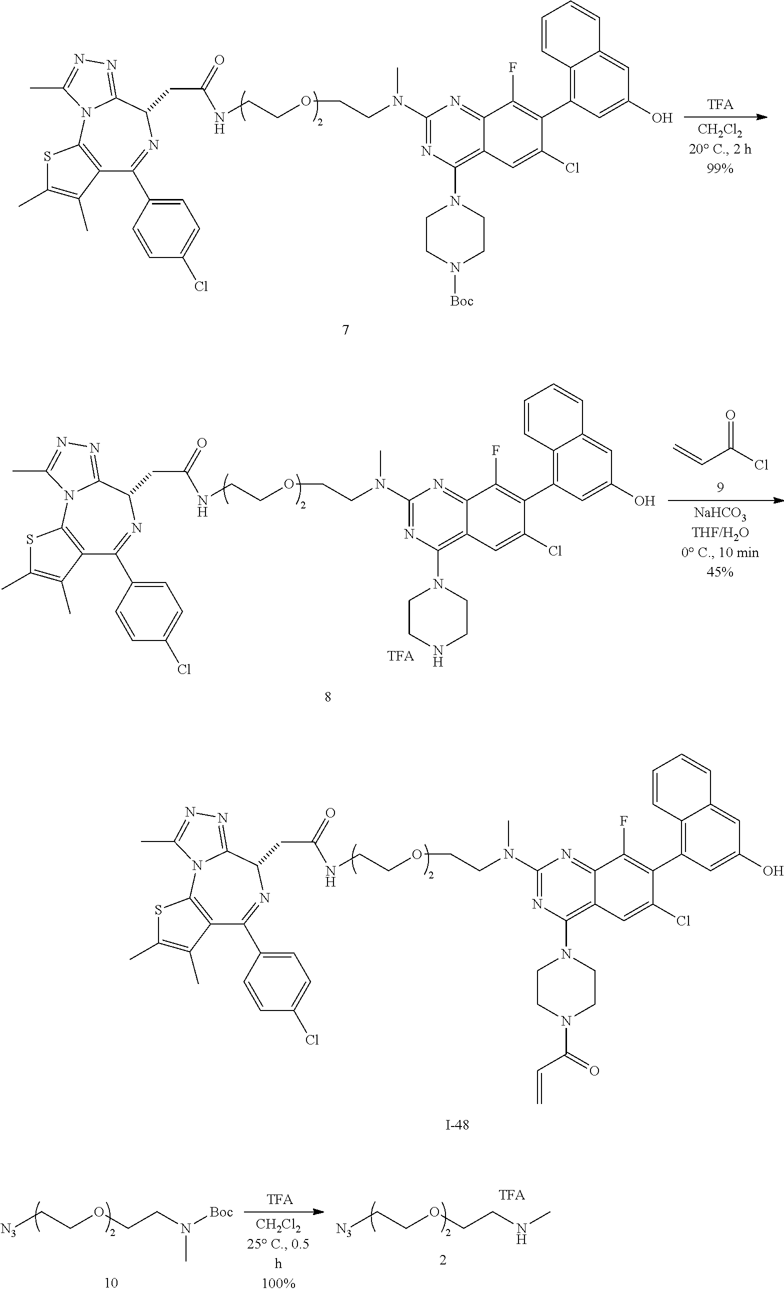

### 48.1 Preparation of Compound 2

A mixture of tert-butyl N-[2-[2-(2-azidoethoxy)ethoxy]ethyl]-N-methyl-carbamate (330 mg, 1.14 mmol, 1.0 equiv) in TFA (2 mL) and CH₂Cl₂ (4 mL) was stirred at 20° C. for 0.5 h. The reaction mixture was concentrated in vacuo to afford 2-[2-(2-azidoethoxy)ethoxy]-N-methyl-ethanamine (345 mg, 1.14 mmol, 100% yield) as a yellow oil. ¹HNMR (400 MHz, DMSO-d₆) δ8.53-8.40 (brs, 2H), 3.66-3.64 (m, 2H), 3.62-3.56 (m, 6H), 3.42-3.39 (m, 2H), 3.12-3.06 (m, 2H), 2.59-2.56 (m, 3H).

### 48.2 Preparation of Compound 3

A mixture of tert-butyl 4-(7-bromo-2,6-dichloro-8-fluoro-quinazolin-4-yl)piperazine-1-carboxylate (450 mg, 0.94 mmol, 1.0 equiv), 2-[2-(2-azidoethoxy)ethoxy]-N-methyl-ethanamine (340 mg, 1.12 mmol, 1.2 equiv) and DIPEA (606 mg, 4.69 mmol, 5.0 equiv) in i-PrOH (3 mL) was stirred at 95° C. for 12 h. The reaction mixture was diluted with water (20 mL) and extracted with EtOAc (10 mL*2). The combined organic phase was washed with brine (10 mL), dried over anhydrous Na₂SO₄, filtered and concentrated in vacuo. The residue was purified by column chromatography on silica gel (Petroleum ether/Ethyl acetate=10/1 to 4/1) to afford tert-butyl 4-[2-[2-[2-(2-azidoethoxy)ethoxy]ethyl-methyl-amino]-7-bromo-6-chloro-8-fluoro-quinazolin-4-yl]piperazine-1-carboxylate (440 mg, 682 μmol, 73% yield, 98% purity) as a yellow oil. ¹H NMR (400 MHz, CDCl₃) δ7.57 (d, 1H, J=1.6 Hz), 3.97-3.85 (m, 2H), 3.83-3.73 (m, 2H), 3.72-3.59 (m, 14H), 3.42-3.36 (m, 2H), 3.32 (brs, 3H), 1.52 (s, 9H). LC-MS: MS (ES′): RT=0.881 min, m/z=631.1 [M+H⁺].

### 48.3 Preparation of Compound 5

To a mixture of tert-butyl 4-[2-[2-[2-(2-azidoethoxy)ethoxy]ethyl-methyl-amino]-7-bromo-6-chloro-8-fluoro-quinazolin-4-yl]piperazine-1-carboxylate (440 mg, 696 μmol, 1.0 equiv), 4-(4,4,5,5-tetramethyl-1,3,2-dioxaborolan-2-yl)naphthalen-2-ol (226 mg, 835 μmol, 1.2 equiv), Pd(PPh₃)₄(402 mg, 348 μmol, 0.5 equiv) and KF (101 mg, 1.74 mmol, 2.5 equiv) in H₂O (2 mL) and dioxane (10 mL) stirred at 90° C. for 3 h under N₂. The reaction mixture was poured into H₂O (30 mL) and extracted with ethyl acetate (20 mL*2). The combined organic phase was washed with brine (15 mL), dried over anhydrous Na₂SO₄, filtered and concentrated in vacuo. The residue was purified by column chromatography on silica gel (Dichloromethane/Methanol=50/1 to 10/1) to afford tert-butyl 4-[2-[2-[2-(2-aminoethoxy)ethoxy]ethyl-methyl-amino]-6-chloro-8-fluoro-7-(3-hydroxy-1-naphthyl)quinazolin-4-yl]piperazine-1-carboxylate (220 mg, 87% purity) as a brown oil. ¹H NMR (400 MHz, CDCl₃) δ7.65 (d, 1H, J=8.0 Hz), 7.54 (s, 1H), 7.36-7.28 (m, 2H), 7.19-7.10 (m, 2H), 7.07 (d, 1H, J=2.4 Hz), 3.74-3.30 (m, 20H), 3.19 (s, 3H), 2.83-2.79 (m, 2H), 1.44 (s, 9H). LC-MS: MS (ES⁺): RT=0.833 min, m/z=699.3 [M+H⁺];

### 48.4 Preparation of Compound 7

A mixture of tert-butyl 4-[2-[2-[2-(2-aminoethoxy)ethoxy]ethyl-methyl-amino]-6-chloro-8-fluoro-7-(3-hydroxy-1-naphthyl)quinazolin-4-yl]piperazine-1-carboxylate (220 mg, 286 μmol, 87% purity, 1.0 equiv), 2-[(9S)-7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetic acid (103 mg, 257 mol, 0.9 equiv), EDCI (164 mg, 858 μmol, 3.0 equiv), HOBt (77.3 mg, 572 μmol, 2.0 equiv) and DIEA (222 mg, 1.72 mmol, 6.0 equiv) in DMF (5 mL) was stirred at 20° C. for 12 h under N₂. The reaction mixture was diluted with brine (20 mL) and extracted with EtOAc (15 mL*3). The combined organic phase was dried over anhydrous Na₂SO₄, filtered and concentrated in vacuo. The residue was purified by prep-HPLC (column: Phenomenex Gemini-NX C18 75*30 mm*3 um; mobile phase: [water(0.225% FA)-ACN]; B%: 42%-72%, 7 min) to afford tert-butyl 4-[6-chloro-2-[2-[2-1[2-[[2-[(9S)-7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetyl]amino]ethoxy]ethoxy]ethyl-methyl-amino]-8-fluoro-7-(3-hydroxy-1-naphthyl)quinazolin-4-yl]piperazine-1-carboxylate (200 mg, 175 μmol, 61% yield) as a yellow solid. ¹H NMR (400 MHz, CDCl₃) δ7.63 (t, 1H, J=8.0 Hz), 7.56 (brs, 1H), 7.34-7.18 (m, 9H), 7.15-7.07 (m, 1H), 6.84-6.62 (m, 1H), 4.63-4.51 (m, 1H), 3.77-3.18 (m, 25H), 2.60 (d, 3H, J=4.4 Hz), 2.30 (d, 3H, J=7.6 Hz), 1.57 (d, 3H, J=3.6 Hz), 1.43 (s, 9H). LC-MS: MS (ES⁺): RT=0.722 min, m/z=526.8 [M/2+H⁺].

### 48.5 Preparation of Compound 8

Prepared similarly to compound 9 in Compound I-44. The reaction mixture was concentrated in vacuo to afford N-[2-[2-[2-[[6-chloro-8-fluoro-7-(3-hydroxy-1-naphthyl)-4-piperazin-1-yl-quinazolin-2-yl]-methyl-amino]ethoxy]ethoxy]ethyl]-2-[(9S)-7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetamide (100 mg, 93.8 μmol, 99% yield, TFA salt) as a yellow oil. LC-MS: MS (ES⁺): RT=0.773 min, m/z=951.1 [M+H⁺].

### 48.6 Preparation of Compound I-48

To a mixture of N-[2-[2-[2-[[6-chloro-8-fluoro-7-(3-hydroxy-1-naphthyl)-4-piperazin-1-yl-quinazolin-2-yl]-methyl-amino]ethoxy]ethoxy]ethyl]-2-[(9S)-7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetamide (100 mg, 93.8 μmol, 1.0 equiv, TFA salt) and NaHCO₃ (78.8 mg, 938 μmol, 10 equiv) in H₂O (1 mL) and THF (5 mL) was added a solution of prop-2-enoyl chloride (8.49 mg, 93.8 μmol, 1.0 equiv) in THF (0.5 mL) at 0° C. The reaction mixture was stirred at 0° C. for 10 min. The reaction mixture was diluted with water (8 mL) and extracted with ethyl acetate (6 mL*3). The combined organic phase was washed with brine (5 mL), dried with anhydrous Na₂SO₄, filtered and concentrated in vacuo. The residue was purified by prep-HPLC (column: Phenomenex Gemini-NX C18 75*30 mm*3 um; mobile phase: [water(0.225% FA)-ACN]; B%: 32%-62%, 7 min) to afford N-[2-[2-[2-[[6-chloro-8-fluoro-7-(3-hydroxy-1-naphthyl)-4-(4-prop-2-enoylpiperazin-1-yl)quinazolin-2-yl]-methyl-amino]ethoxy]ethoxy]ethyl]-2-[(9S)-7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetamide (42.0 mg, 41.7 μmol, 45% yield, 100% purity) as a white solid. ¹HNMR (400 MHz, CDCl3) δ7.77-7.63 (m, 2H), 7.47-7.35 (m, 4H), 7.34-7.27 (m, 5H), 7.24-7.15 (m, 1H), 7.03-6.81 (m, 1H), 6.66-6.55 (m, 1H), 6.43-6.33 (m, 1H), 5.82-5.74 (m, 1H), 4.72-4.61 (m, 1H), 3.98-3.72 (m, 12H), 3.67-3.34 (m, 10H), 3.33-3.25 (m, 3H), 2.75-2.64 (m, 3H), 2.45-2.35 (m, 3H), 1.71-1.63 (m, 3H). LC-MS: MS (ES′): RT=1.604 min, m/z=1005.5 [M+H⁺]; LC-MS method 40.

### Example 49—Synthesis of Compound I-49

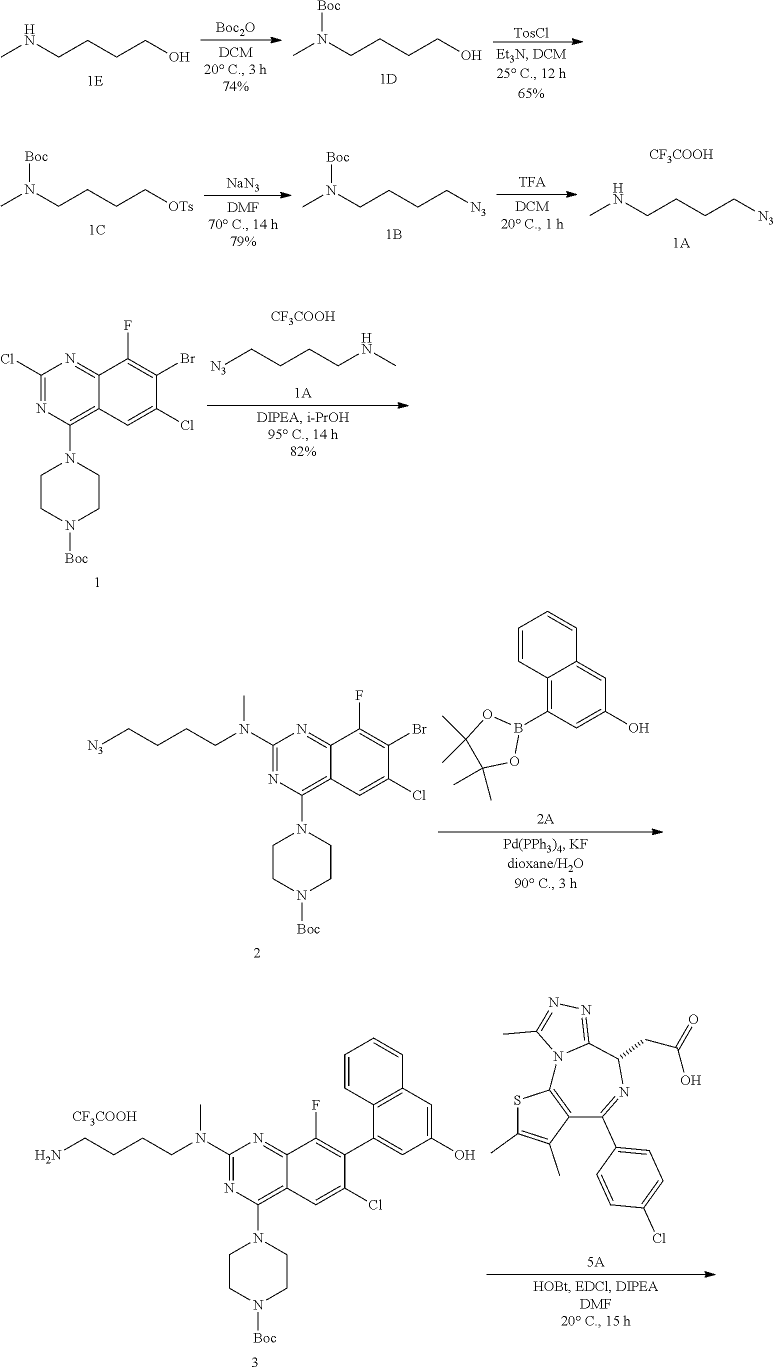

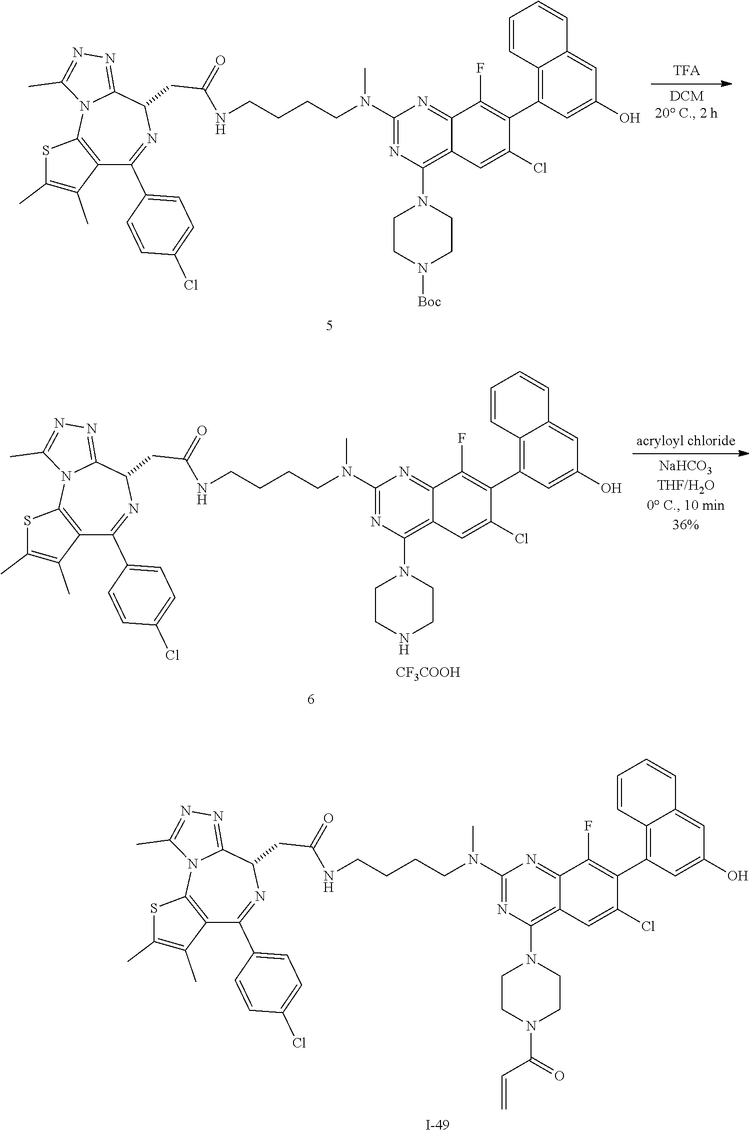

### 49.1 Preparation of Compound 1D

To a solution of 4-(methylamino)butan-1-ol (0.73 g, 7.11 mmol, 1.0 equiv) in DCM (20 mL) was added Boc₂O (1.55 g, 7.11 mmol, 1.0 equiv). The mixture was stirred at 20° C. for 3 h. The reaction mixture was concentrated in vacuo. The residue was purified by column chromatography on silica gel (Dichloromethane/Methanol=I/O to 10/1) to afford tert-butyl N-(4-hydroxybutyl)-N-methyl-carbamate (1.07 g, 5.26 mmol, 74% yield) as a colorless oil. ¹H NMR (400 MHz, DMSO-d₆): δ4.38 (t, 1H, J=5.2 Hz), 3.39 (q, 2H, J=6.0 Hz), 3.14 (t, 2H, J=7.2 Hz), 2.74 (brs, 3H), 1.50-1.43 (m, 2H), 1.38 (s, 9H), 1.37-1.30 (m, 2H).

### 49.2 Preparation of Compound 1C

To a solution of tert-butyl N-(4-hydroxybutyl)-N-methyl-carbamate (1.07 g, 5.26 mmol, 1.0 equiv) in DCM (10 mL) were added Et₃N (1.60 g, 15.8 mmol, 3.0 equiv) and TosCl (1.51 g, 7.90 mmol, 1.5 equiv). The mixture was stirred at 25° C. for 12 h. The reaction mixture was diluted with water (10 mL) and extracted with DCM (15 mL*3). The combined organic phase was washed with brine (15 mL), dried over anhydrous Na₂SO₄, filtered and concentrated in vacuo. The residue was purified by column chromatography on silica gel (Dichloromethane/Methanol=I/O to 20/1) to afford 4-[tert-butoxycarbonyl(methyl)amino]butyl 4-methylbenzenesulfonate (1.23 g, 3.44 mmol, 65% yield) as a colorless oil. ¹H NMR (400 MHz, CDCl₃): δ7.79 (d, 2H, J=8.4 Hz), 7.35 (d, 2H, J=8.0 Hz), 4.05 (t, 2H, J=6.0 Hz), 3.18 (t, 2H, J=6.8 Hz), 2.79 (s, 3H), 2.46 (s, 3H), 1.69-1.62 (m, 2H), 1.58-1.50 (m, 2H), 1.43 (s, 9H). LC-MS: MS (ES′): RT=0.983 min, m/z=380.1 [M+Na+].

### 49.3 Preparation of Compound 1B

To a solution of 4-[tert-butoxycarbonyl(methyl)amino]butyl 4-methyl-benzenesulfonate (1.13 g, 3.16 mmol, 1.0 equiv) in DMF (10 mL) was added NaN₃ (616 mg, 9.48 mmol, 3.0 equiv). The mixture was stirred at 70° C. for 14 h. The pH of the reaction mixture was adjusted to 9 by addition saturated aqueous Na₂CO₃ solution and the mixture was extracted with DCM/MeOH (10/1, 25 mL*3). The combined organic phase was washed with brine (20 mL), dried over anhydrous Na₂SO₄, filtered and concentrated in vacuo to afford tert-butyl N-(4-azidobutyl)-N-methyl-carbamate (570 mg, 2.50 mmol, 79% yield) as a yellow oil. ¹H NMR (400 MHz, CDCl₃): δ3.30-3.28 (m, 2H), 3.27-3.21 (m, 2H), 2.84 (s, 3H), 1.61-1.55 (m, 4H), 1.46 (s, 9H).

### 49.4 Preparation of Compound 1A

Prepared similarly to compound 9 in Compound I-44. The reaction mixture was concentrated in vacuo to afford 4-azido-N-methyl-butan-1-amine (320 mg, crude, TFA salt) as a yellow oil. ¹H NMR (400 MHz, DMSO-d₆): δ8.41 (brs, 2H), 3.37 (t, 2H, J=6.4 Hz), 3.00-2.80 (m, 2H), 2.55 (t, 3H, J=5.6 Hz), 1.64-1.55 (m, 4H).

### 49.5 Preparation of Compound 2

To a solution of tert-butyl 4-(7-bromo-2,6-dichloro-8-fluoro-quinazolin-4-yl)piperazine-1-carboxylate (500 mg, 1.04 mmol, 1.0 equiv) and 4-azido-N-methyl-butan-1-amine (303 mg, 1.25 mmol, 1.2 equiv, TFA salt) in i-PrOH (5 mL) was added DIPEA (673 mg, 5.21 mmol, 5.0 equiv). The mixture was stirred at 95° C. for 14 h. The reaction mixture was concentrated in vacuo. The residue was purified by column chromatography on silica gel (petroleum ether/ethyl acetate=10/1 to 3/1) to afford tert-butyl 4-[2-[4-azidobutyl (methyl)amino]-7-bromo-6-chloro-8-fluoro-quinazolin-4-yl]piperazine-1-carboxylate (490 mg, 0.86 mmol, 82% yield) as a yellow oil. ¹H NMR (400 MHz, CDCl₃): δ7.55-7.54 (m, 1H), 3.75 (brs, 2H), 3.63-3.61 (m, 8H), 3.36 (brs, 2H), 3.23 (brs, 3H), 1.50 (s, 9H), 0.92-0.83 (m, 4H). LC-MS: MS (ES⁺): RT=0.758 min, m/z=573.0 [M+H⁺].

### 49.6 Preparation of Compound 3

Prepared similarly to compound 5 in Compound I-48. The residue was purified by prep-HPLC (TFA condition:column: Phenomenex luna C18 150*40 mm* 15 um; mobile phase: [water(0.1%TFA)-ACN]; B%: 18%-48%, 11 min) to afford tert-butyl 4-[2-[4-aminobutyl(methyl)amino]-6-chloro-8-fluoro-7-(3-hydroxy-1-naphthyl)quinazolin-4-yl]piperazine-1-carboxylate (400 mg, crude, TFA salt) as a yellow oil. ¹H NMR (400 MHz, DMSO-d₆): δ7.81-7.79 (m, 2H), 7.65 (brs, 3H), 7.46-7.42 (m, 1H), 7.27 (d, 1H, J=2.4 Hz), 7.22-7.21 (m, 2H), 7.04 (d, 1H, J=2.4 Hz), 3.76-3.72 (m, 5H), 3.61-3.57 (m, 4H), 3.17 (s, 3H), 2.87-2.79 (m, 2H), 2.55-2.54 (m, 2H), 1.71-1.61 (m, 2H), 1.55-1.53 (m, 2H), 1.44 (s, 9H). LC-MS: MS (ES⁺): RT=0.575 min, m/z=609.2 [M+H⁺].

### 49.7 Preparation of Compound 5

A mixture of 2-[7-(4-chlorophenyl)-4,5,13-trimethyl-3-thia-1,8,11,12-tetrazatricyclo[8.3.0.02,6]trideca-2(6), 4,7,10,12-pentaen-9-yl]acetic acid (132 mg, 0.33 mmol, 1.0 equiv), tert-butyl 4-[2-[4-aminobutyl(methyl)amino]-6-chloro-8-fluoro-7-(3-hydroxy-1-naphthyl)quinazolin-4-yl]piperazine-1-carboxylate (238 mg, 0.33 mmol, 1.0 equiv, TFA salt), EDCI (158 mg, 0.82 mmol, 2.5 equiv), HOBt (111 mg, 0.82 mmol, 2.5 equiv) and DIPEA (426 mg, 3.29 mmol, 0.57 mL, 10.0 equiv) in DMF (3 mL) was stirred at 20° C. for 15 h under N₂. The reaction mixture was diluted with water (5 mL) and extracted with ethyl acetate (10 mL*3). The combined organic phase was washed with brine (15 mL), dried over anhydrous Na₂SO₄, filtered and concentrated in vacuo. The residue was purified by prep-HPLC (FA condition:column: Phenomenex Luna C18 150*25 mm*10 um; mobile phase: [water(0.225% FA)-ACN]; B%: 55%-85%, 2 min) to afford tert-butyl 4-[6-chloro-2-[4-[[2-[7-(4-chlorophenyl)-4,5,13-trimethyl-3-thia-1,8,11,12-tetrazatricyclo [8.3.0.02,6]trideca-2(6), 4,7,10,12-pentaen-9-yl]acetyl]amino]butyl-methyl-amino]-8-fluoro-7-(3-hydroxy-1-naphthyl)quinazolin-4-yl]piperazine-1-carboxylate (118 mg, crude) as a yellow oil. LC-MS: MS (ES⁺): RT=0.735 min, m/z=991.2 [M+H⁺].

### 49.8 Preparation of Compound 6

Prepared similarly to compound 9 in Compound I-44. The reaction mixture was concentrated in vacuo to afford N-[4-[[6-chloro-8-fluoro-7-(3-hydroxy-1-naphthyl)-4-piperazin-1-yl-quinazolin-2-yl]-methyl-amino]butyl]-2-[7-(4-chlorophenyl)-4,5,13-trimethyl-3-thia-1,8,11,12-tetrazatricyclo[8.3.0.02,6]trideca-2(6), 4,7,10,12-pentaen-9-yl]acetamide (120 mg, crude, TFA salt) as a yellow oil. LC-MS: MS (ES⁺): RT=0.847 min, m/z=891.4 [M+H⁺].

### 49.9 Preparation of Compound I-49

To a solution of N-[4-[[6-chloro-8-fluoro-7-(3-hydroxy-1-naphthyl)-4-piperazin-1-yl-quinazolin-2-yl]-methyl-amino]butyl]-2-[(9S)-7-(4-chlorophenyl)-4,5,13-trimethyl-3-thia-1,8,11,12-tetrazatricyclo[8.3.0.02,6]trideca-2(6), 4,7,10,12-pentaen-9-yl]acetamide (106 mg, 0.12 mmol, 1.0 equiv, TFA salt) and NaHCO₃ (100 mg, 1.19 mmol, 10.0 equiv) in THF (4 mL) and H₂O (0.8 mL) was added a solution of prop-2-enoyl chloride (10.8 mg, 0.12 mmol, 1.0 equiv) at 0° C. The mixture was stirred at 0° C. for 10 min. The reaction mixture was diluted with water (5 mL) and extracted with ethyl acetate (10 mL*3). The combined organic phase was washed with brine (15 mL), dried over anhydrous Na₂SO₄, filtered and concentrated in vacuo. The residue was purified by prep-HPLC (FA condition:column: Phenomenex luna C18 150*25 mm* 10 um; mobile phase: [water(0.225% FA)-ACN]; B%: 31%-61%, 2 min) to afford N-[4-[[6-chloro-8-fluoro-7-(3-hydroxy-1-naphthyl)-4-(4-prop-2-enoylpiperazin-1-yl)quinazolin-2-yl]-methylamino]butyl]-2-[(9S)-7-(4-chlorophenyl)-4,5,13-trimethyl-3-thia-1,8,11,12-tetrazatricyclo[8.3.0.02,6]trideca-2(6), 4,7,10,12-pentaen-9-yl]acetamide (40.1 mg, 0.04 mmol, 36% yield, 100% purity) as a yellow solid. ¹H NMR (400 MHz, CDCl₃) δ7.84-7.43 (m, 3H), 7.40-7.30 (m, 4H), 7.26-6.80 (m, 4H), 6.63-6.48 (m, 1H), 6.40-6.35 (m, 1H,), 5.79-5.76 (m, 1H), 4.76-4.63 (m, 1H), 4.27-3.56 (m, 10H), 3.51-3.26 (m, 4H), 3.25-2.87 (m, 4H), 2.70-2.66 (m, 3H), 2.40-2.32 (m, 3H), 1.97-1.67 (m, 3H), 1.64-1.43 (m, 3H). LC-MS: MS (ES⁺): RT=1.509 min, m/z=945.2 [M+H⁺]; LC-MS method 40.

### Example 50—Synthesis of Compound I-50

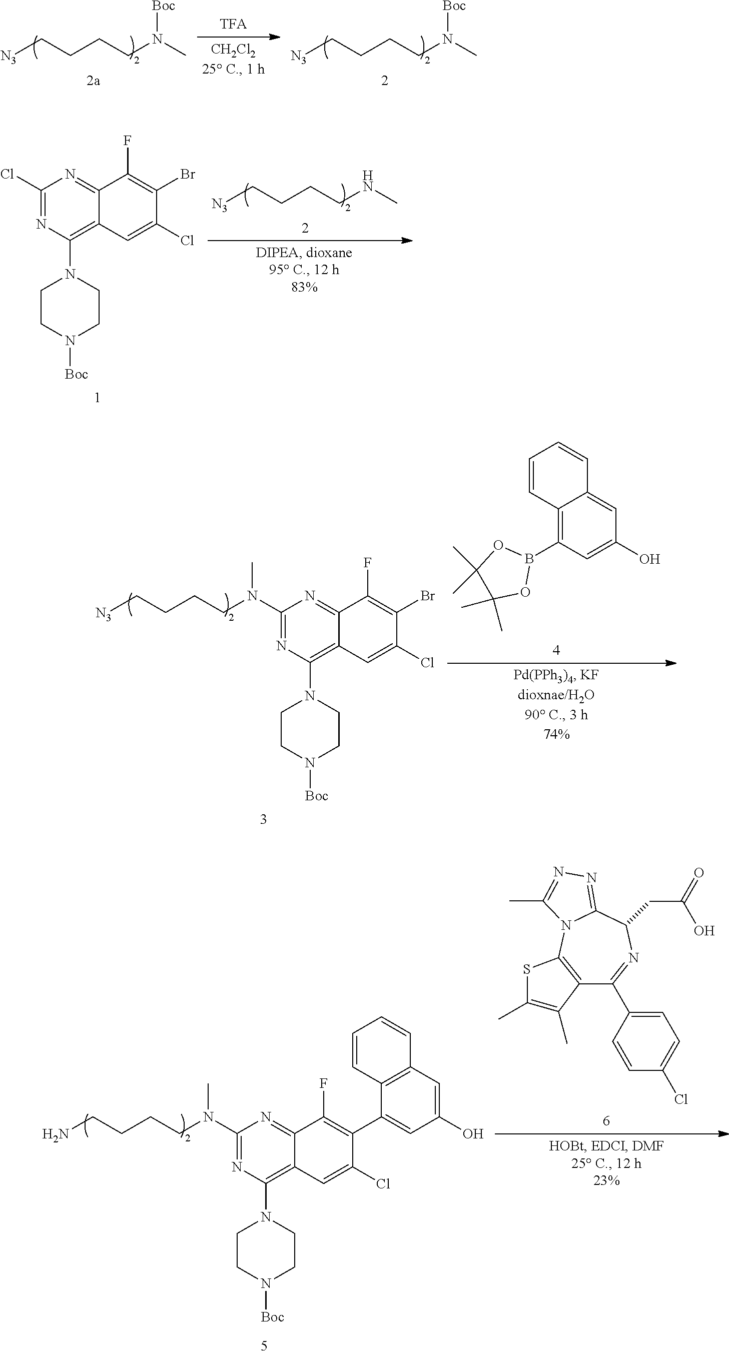

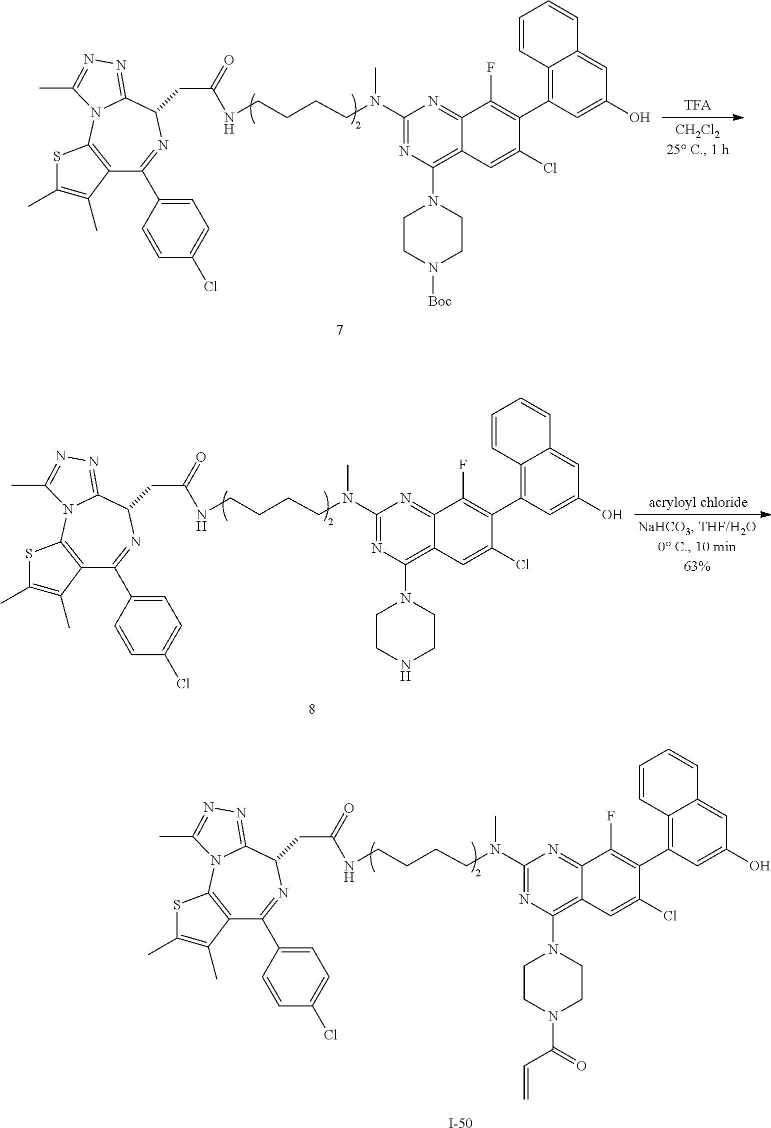

### 50.1 Preparation of Compound 2

A mixture of tert-butyl N-(8-azidooctyl)-N-methyl-carbamate (320 mg, 1.13 mmol, 1.0 equiv) in DCM (4 mL) and TFA (2 mL) was stirred at 25° C. for 1 h. The reaction mixture was concentrated in vacuo to afford 8-azido-N-methyl-octan-1-amine (335 mg, 1.12 mmol, 100% yield, TFA salt) as a brown oil.

### 50.2 Preparation of Compound 2

To a solution of tert-butyl 4-(7-bromo-2,6-dichloro-8-fluoro-quinazolin-4-yl)piperazine-1-carboxylate (450 mg, 937 μmol, 1.0 equiv) in i-PrOH (3 mL) were added DIPEA (606 mg, 4.69 mmol, 0.82 mL, 5.0 equiv) and 8-azido-N-methyl-octan-1-amine (335 mg, 1.12 mmol, 1.2 equiv, TFA salt). The mixture was stirred at 95° C. for 12 h under N₂. The reaction mixture was diluted with water (5 mL) and extracted with DCM/MeOH (10/1.5 mL*5). The combined organic phase was dried with anhydrous Na₂SO₄, filtered and concentrated in vacuo. The residue was purified by column chromatography on silica gel (Petroleum ether/Ethyl acetate=100/1 to 10/1) to afford tert-butyl4-[2-[8-azidooctyl(methyl)amino]-7-bromo-6-chloro-8-fluoro-quinazolin-4-yl]piperazine-1-carboxylate (490 mg, 780 μmol, 83% yield) as a yellow oil. ¹HNMR (400 MHz, CDCl₃-d): S 7.57-7.52 (m, 1H), 3.65-3.57 (m, 8H), 3.28-3.22 (m, 4H), 1.69-1.60 (m, 3H), 1.59-1.55 (m, 1H), 1.50 (s, 9H), 1.37-1.31 (m, 8H), 0.92-0.84 (m, 3H). LC-MS: MS (ES⁺): RT=1.103 min, m/z=629.4 [M+H⁺].

### 50.3 Preparation of Compound 5

Prepared similarly to compound 5 in Compound I-48. The residue was purified by column chromatography on silica gel (DCM/MeOH=100/1 to 10/1 with 0.3% NH₃—H₂O) to afford tert-butyl 4-[2-[8-aminooctyl(methyl)amino]-6-chloro-8-fluoro-7-(3-hydroxy-1-naphthyl)quinazolin-4-yl]piperazine-1-carboxylate (400 mg, 578 μmol, 74% yield) as a white solid. ¹HNMR (400 MHz, DMSO-d₆): δ7.82-7.74 (m, 1H), 7.58 (brs, 1H), 7.46-7.32 (m, 2H), 7.29-7.18 (m, 2H), 7.13-6.95 (m, 1H), 3.82-3.42 (m, 13H), 3.19-3.16 (m, 3H), 3.15-3.13 (m, 2H), 1.60-1.58 (m, 1H), 1.50-1.37 (m, 9H), 1.37-0.78 (m, 11H).

### 50.4 Preparation of Compound 7

Prepared similarly to compound 5 in Compound I-49. The residue was purified by prep-HPLC (column: Phenomenex luna C18 150*25 mm* 10 um; mobile phase: [water(0.225% FA)-ACN]; B%: 62%-82%, 10 min) to afford tert-butyl 4-[6-chloro-2-[8-[[2-[7-(4-chlorophenyl)-4,5,13-trimethyl-3-thia-1,8,11,12-tetrazatricyclo[8.3.0.02,6]trideca-2(6), 4,7,10,12-pentaen-9-yl]acetyl]amino]octyl-methyl-amino]-8-fluoro-7-(3-hydroxy-1-naphthyl)quinazolin-4-yl]piperazine-1-carboxylate (80.0 mg, 76.3 μmol, 23% yield) as a white solid.

¹HNMR (400 MHz, DMSO-d₆): S 8.15 (brs, 1H), 7.82-7.75 (m, 2H), 7.48-7.40 (m, 5H), 7.29-7.19 (m 3H), 7.04 (s, 1H), 4.52-4.49 (m, 1H), 3.72-3.68 (m, 4H), 3.66-3.63 (m, 2H), 3.59-3.55 (m, 4H), 3.28-3.06 (m, 11H), 2.59 (s, 3H), 2.39 (s, 3H), 1.62-1.57 (m, 4H), 1.44 (s, 9H), 1.30-1.26 (m, 8H). LC-MS: MS (ES⁺): RT=0.775 min, m/z=1047.3 [M+H⁺].

### 50.5 Preparation of Compound 8

Prepared similarly to compound 9 in Compound I-44. The reaction mixture was concentrated in vacuo to afford N-[8-[[6-chloro-8-fluoro-7-(3-hydroxy-1-naphthyl)-4-piperazin-1-yl-quinazolin-2-yl]-methyl-amino]octyl]-2-[7-(4-chlorophenyl)-4,5,13-trimethyl-3-thia-1,8,11,12-tetrazatricyclo[8.3.0.02,6]trideca-2(6), 4,7,10,12-pentaen-9-yl]acetamide (75.0 mg, 76.2 μmol, 99% yield, TFA salt) as a brown solid. LC-MS: MS (ES⁺): RT=0.911 min, m/z=947.6 [M+H⁺].

### 50.6 Preparation of Compound I-50

To a solution of N-[8-[[6-chloro-8-fluoro-7-(3-hydroxy-1-naphthyl)-4-piperazin-1-yl-quinazolin -2-yl]-methyl-amino]octyl]-2-[7-(4-chlorophenyl)-4,5,13-trimethyl-3-thia-1,8,11,12-tetrazatricyclo [8.3.0.02,6]trideca-2(6), 4,7,10,12-pentaen-9-yl]acetamide (75.0 mg, 70.6 μmol, 1.0 equiv, TFA salt) in THF (4 mL) and H₂O (0.8 mL) were added NaHCO₃ (59.3 mg, 706 μmol, 27.5 μL, 10 equiv) and prop-2-enoyl chloride (6.39 mg, 70.6 μmol, 5.76 μL, 1.0 equiv) in THF (0.5 mL). The mixture was stirred at 0° C. for 10 min. The reaction mixture was diluted with water (5 mL) and extracted with ethyl acetate (5 mL*3). The combined organic phase was washed with brine (5 mL), dried with anhydrous Na₂SO₄, filtered and concentrated in vacuo. The residue was purified by prep-HPLC (column: Phenomenex luna C18 150*25 mm* 10 um; mobile phase: [water(0.225% FA)-ACN]; B%: 45%-75%, 2 min) to afford N-[8-[1[6-chloro-8-fluoro-7-(3-hydroxy-1-naphthyl)-4-(4-prop-2-enoylpiperazin-1-yl)quinazolin-2-yl]-methyl-amino]octyl]-2-[7-(4-chlorophenyl)-4,5,13-trimethyl-3-thia-1,8,11,12-tetrazatricyclo[8.3.0.02,6]trideca-2(6), 4,7,10,12-pentaen-9-yl]acetamide (45.0 mg, 44.6 μmol, 63% yield, 99% purity) as a yellow solid. ‘HNMR (400 MHz, DMSO-d₆): S 8.15 (brs, 1H), 7.88-7.73 (m, 2H), 7.49-7.39 (m, 5H), 7.29-7.19 (m, 3H), 7.10-6.99 (m, 1H), 6.94-6.77 (m, 1H), 6.26-6.10 (m, 1H), 5.80-5.66 (m, 1H), 4.56-4.44 (m, 1H), 3.88-3.73 (m, 7H), 3.70-3.58 (m, 3H), 3.23-3.05 (m, 8H), 2.59 (s, 3H), 2.39 (s, 3H), 1.66-1.52 (m, 5H), 1.50-1.34 (m, 3H), 1.33-1.23 (m, 7H). LC-MS: MS (ES⁺): RT=2.855 min, m/z=1001.2 [M+H⁺].

### Example 51—Synthesis of Compound I-51

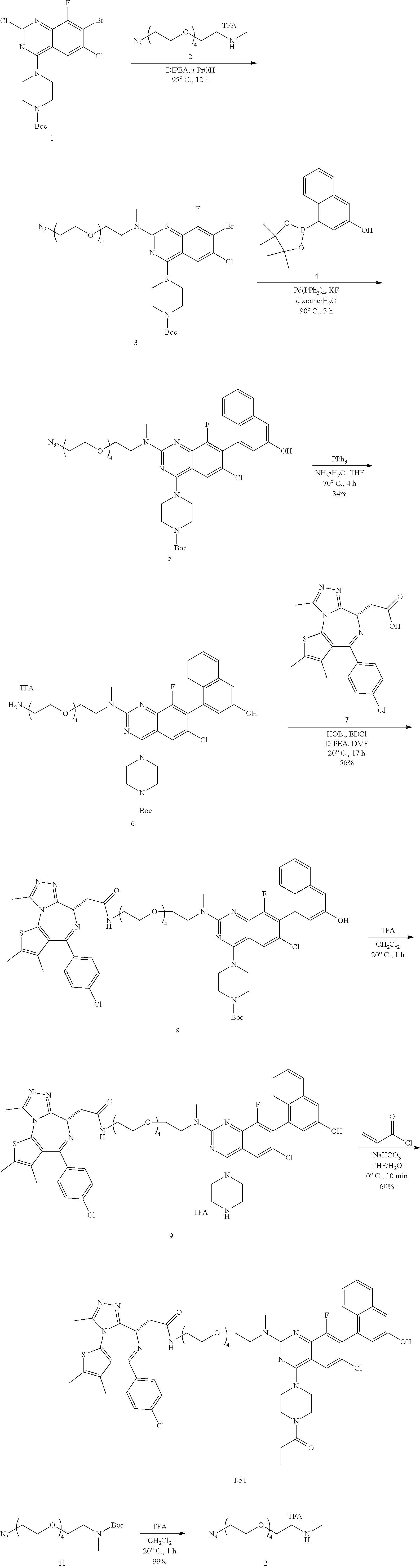

### 51.1 Preparation of Compound 2

Prepared similarly to compound 9 in Compound I-44. The reaction mixture was concentrated in vacuo to afford 2-[2-[2-[2-(2-azidoethoxy)ethoxy]ethoxy]ethoxy]-N-methyl-ethanamine (144 mg, 0.37 mmol, 99% yield, TFA salt) as a colorless oil. ¹HNMR (400 MHz, DMSO-d₆): δ8.40 (brs, 2H), 3.76-3.38 (m, 18H), 3.12-3.08 (m, 2H), 2.59-2.56 (m, 3H).

### 51.2 Preparation of Compound 3

Prepared similarly to compound 2 in Compound I-50. The residue was purified by column chromatography on silica gel (Petroleum ether/EtOAc=4/1 to 2/1) to afford tert-butyl 4-[2-[2-[2-[2-1[2-(2-azidoethoxy)ethoxy]ethoxy]ethoxy]ethyl-methyl-amino]-7-bromo-6-chloro-8-fluoro-quinazolin-4-yl]piperazine-1-carboxylate (270 mg, crude) as a yellow oil. ¹HNMR (400 MHz, CDCl₃): δ7.56 (s, 1H), 3.94-3.87 (m, 2H), 3.80-3.56 (m, 24H), 3.41-3.37 (m, 2H), 3.33 (brs, 3H), 1.50 (s, 9H).

### 51.3 Preparation of Compound 5

A mixture tert-butyl 4-[2-[2-[2-[2-[2-(2-azidoethoxy)ethoxy]ethoxy]ethoxy]ethyl-methyl-amino]-7-bromo-6-chloro-8-fluoro-quinazolin-4-yl]piperazine-1-carboxylate (270 mg, 0.37 mmol, 1.0 equiv), 4-(4,4,5,5-tetramethyl-1,3,2-dioxaborolan-2-yl)naphthalen-2-ol (152 mg, 0.56 mmol, 1.5 equiv), Pd(PPh₃)₄(217 mg, 0.19 mmol, 0.5 equiv) and KF (54.5 mg, 0.94 mmol, 2.5 equiv) in dioxane (5 mL) and H₂O (1 mL) was stirred at 90° C. for 3 h. The reaction mixture was diluted with water (15 mL) and the mixture was extracted with ethyl acetate (15 mL*3). The combined organic phase was washed with brine (15 mL), dried with anhydrous Na₂SO₄, filtered and concentrated in vacuo to afford tert-butyl 4-[2-[2-[2-[2-[2-(2-azidoethoxy)ethoxy]ethoxy]ethoxy]ethyl-methyl-amino]-6-chloro-8-fluoro-7-(3-hydroxy-1-naphthyl)quinazolin-4-yl]piperazine-1-carboxylate (0.54 g, 54% purity) as a brown solid. LC-MS: MS (ES⁺): RT=0.911 min, m/z=783.3 [M+H⁺].

### 51.4 Preparation of Compound 6

To a mixture of tert-butyl 4-[2-[2-[2-[2-[2-(2-azidoethoxy)ethoxy]ethoxy]ethoxy]ethyl-methyl-amino]-6-chloro-8-fluoro-7-(3-hydroxy-1-naphthyl)quinazolin-4-yl]piperazine-1-carboxylate (500 mg, 0.34 mmol, 54% purity, 1.0 equiv) in THF (8 mL) and 28% NH₃.H₂O (2 mL) was added PPh₃ (181 mg, 0.69 mmol, 2.0 equiv) at 20° C. The reaction mixture was stirred at 70° C. for 4 h. The reaction mixture was concentrated in vacuo. The residue was purified by prep-HPLC (column: Phenomenex luna C18 150*40 mm*15 um; mobile phase: [water(0.1%TFA)-ACN]; B%: 18%-48%, 11 min) to afford tert-butyl 4-[2-[2-[2-[2-[2-(2-aminoethoxy)ethoxy]ethoxy]ethoxy]ethyl-methyl-amino]-6-chloro-8-fluoro-7-(3-hydroxy-1-naphthyl)quinazolin-4-yl]piperazine-1-carboxylate (115 mg, 0.12 mmol, 34% yield, TFA salt) as a yellow solid. LC-MS: MS (ES⁺): RT=0.581 min, m/z=757.2 [M+H⁺].

### 51.5 Preparation of Compound 8

Prepared similarly to compound 5 in Compound I -49. The residue was purified by prep-HPLC (column: Phenomenex Gemini-NX C18 75*30 mm*3 um; mobile phase: [water(0.225% FA)-ACN]; B%: 42%-72%, 7 min) to afford tert-butyl 4-[6-chloro-2-[2-[2-[2-[2-[2-[[2-1[(9S)-7-(4-chlorophenyl)-4,5,13-trimethyl-3-thia-1,8,11,12-tetrazatricyclo[8.3.0.02,6]trideca-2(6), 4,7,10,12-pentaen-9-yl]acetyl]amino]ethoxy]ethoxy]ethoxy]ethoxy]ethyl-methyl-amino]-8-fluoro-7-(3-hydroxy-1-naphthyl)quinazolin-4-yl]piperazine-1-carboxylate (80.0 mg, 66.0 μmol, 56% yield) as a yellow solid. ¹HNMR (400 MHz, CDCl₃): δ7.73-7.68 (m, 1H), 7.65-7.63 (m, 1H), 7.43-7.38 (m, 3H), 7.36-7.30 (m, 5H), 7.22-7.18 (m, 2H), 4.74-4.68 (m, 1H), 3.83-3.64 (m, 14H), 3.62-3.44 (m, 16H), 3.31-3.28 (m, 3H), 2.71-2.68 (m, 3H), 2.40 (3H, s), 1.68-1.65 (m, 3H), 1.52 (9H, s). LC-MS: MS (ES′): RT=0.987 min, m/z=1139.8 [M+H⁺].

### 51.6 Preparation of Compound 9

Prepared similarly to compound 9 in Comopund I-44. The reaction mixture was concentrated in vacuo to afford N—[[2[2-[2-[2-2-[[6-chloro-8-fluoro-7-(3-hydroxy-1-naphthyl)-4-piperazin-1-yl-quinazolin-2-yl]-methyl-amino]ethoxy]ethoxy]ethoxy]ethoxy]ethyl]-2-[(9S)-7-(4-chlorophenyl)-4,5,13-trimethyl-3-thia-1,8,11,12-tetrazatricyclo[8.3.0.02,6]trideca-2(6), 4,7,10,12-pentaen-9-yl]acetamide (76.0 mg, crude, TFA salt) as a yellow oil. LC-MS: MS (ES′): RT=0.874 min, m/z=1039.7 [M+H⁺].

### 51.7 Preparation of Compound I-51

To a mixture of N-[2-[2-[2-[2-[2-[[6-chloro-8-fluoro-7-(3-hydroxy-1-naphthyl)-4-piperazin-1-yl-quinazolin-2-yl]-methyl-amino]ethoxy]ethoxy]ethoxy]ethoxy]ethyl]-2-[(9S)-7-(4-chlorophenyl)-4,5,13-trimethyl-3-thia-1,8,11,12-tetrazatricyclo[8.3.0.02,6]trideca-2(6), 4,7,10,12-pentaen-9-yl]acetamide (76.0 mg, 65.9 μmol, 1.0 equiv, TFA salt) and NaHCO₃ (55.3 mg, 659 μmol, 10.0 equiv) in THF (4 mL) and H₂O (0.8 mL) was added a solution of prop-2-enoyl chloride (5.96 mg, 65.9 μmol, 1.0 equiv) in THF (0.5 mL) at 0° C. The reaction mixture was stirred at 0° C. for 10 min. The reaction mixture was diluted with water (5 mL) and the mixture was extracted with ethyl acetate (5 mL*3). The combined organic phase was washed with brine (5 mL), dried with anhydrous Na₂SO₄, filtered and concentrated in vacuo. The residue was purified by prep-HPLC (column: Phenomenex Gemini-NX C18 75*30 mm*3 um; mobile phase: [water(0.225% FA)-ACN]; B %: 32%-62%, 7 min) to afford N-(2-(4-(4-acryloylpiperazin-1-yl)-6-chloro-8-fluoro-7-(3-hydroxynaphthalen-1-yl)quinazolin-2-yl)-5,8,11,14-tetraoxa-2-azahexadecan-16-yl)-2-((S)-4-(4-chlorophenyl)-2,3,9-trimethyl-6H-thieno[3,2-f][1,2,4]triazolo[4,3-a][1,4]diazepin-6-yl)acetamide (45.0 mg, 40.3 μmol, 61% yield, 98.2% yield) as an off-white solid. ¹HNMR (400 MHz, CDCl₃): δ 7.73-7.68 (m, 1H), 7.64-7.62 (m, 1H), 7.43-7.30 (m, 7H), 7.24-7.18 (m, 2H), 7.07-7.00 (m, 1H), 6.67-6.60 (m, 1H), 6.41-6.36 (m, 1H), 5.80-5.77 (m, 1H), 4.74-4.68 (m, 1H), 3.96-3.91 (m, 2H), 3.85-3.69 (m, 9H), 3.64-3.35 (m, 19H), 3.32-3.30 (m, 3H), 2.71-2.69 (m, 3H), 2.40 (3H, s), 1.68-1.64 (m, 3H). LC-MS: MS (ES′): RT=0.987 min, m/z=1139.8 [M+H⁺]; LC-MS method 40.

### Example 52—Synthesis of Compound I-52

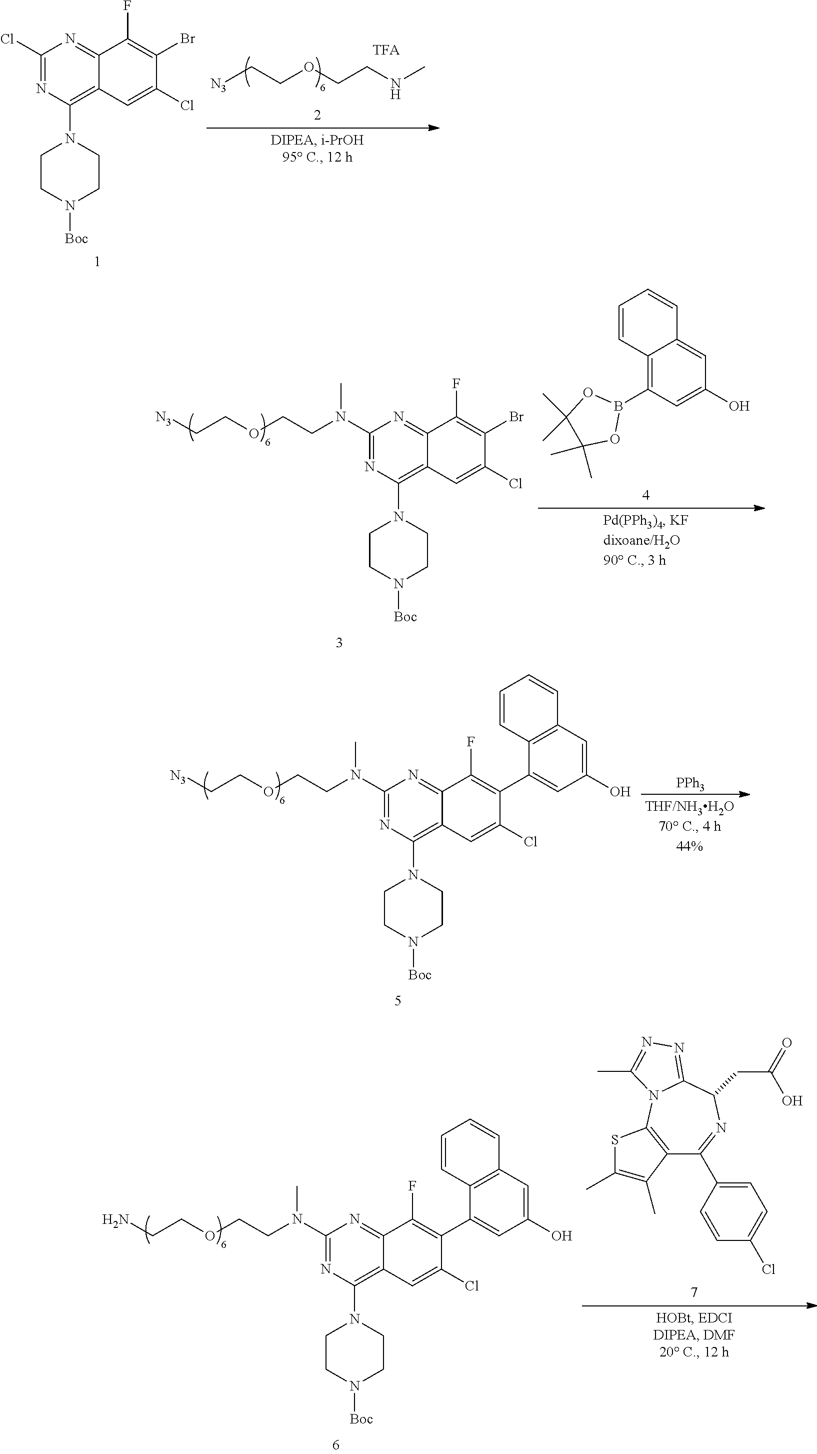

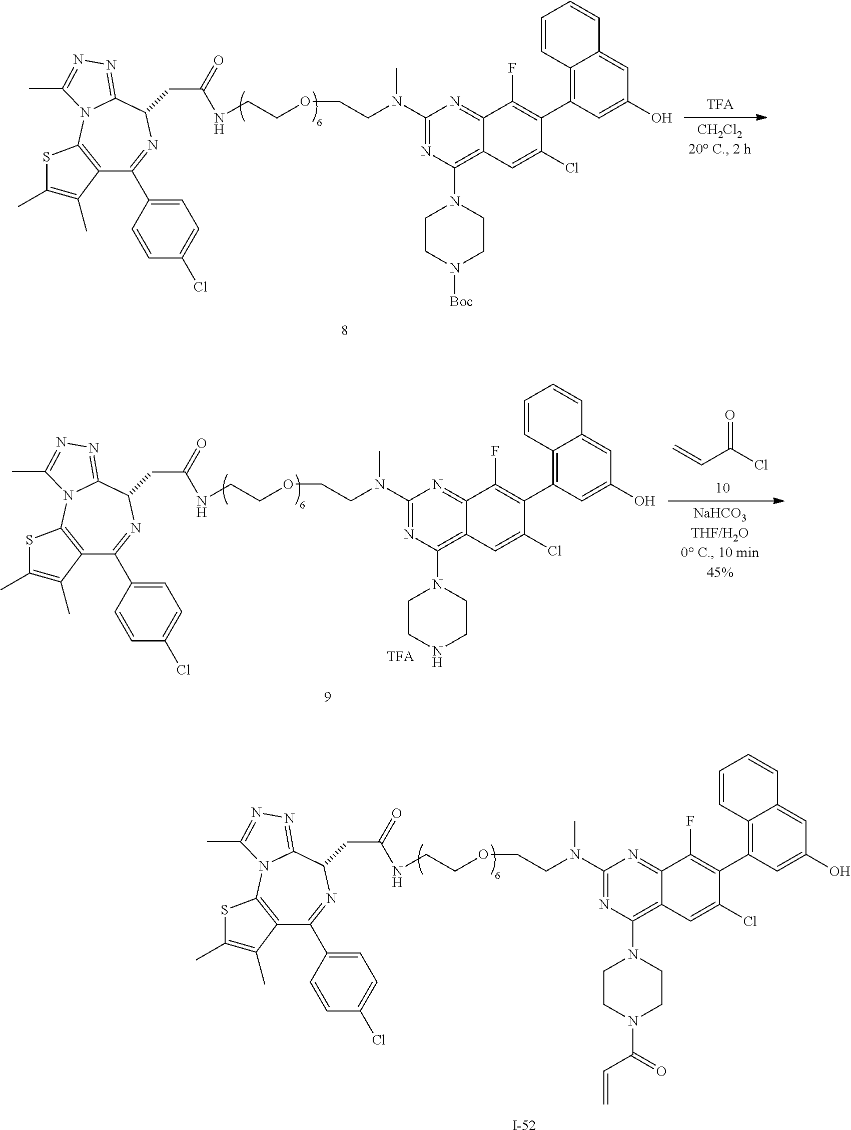

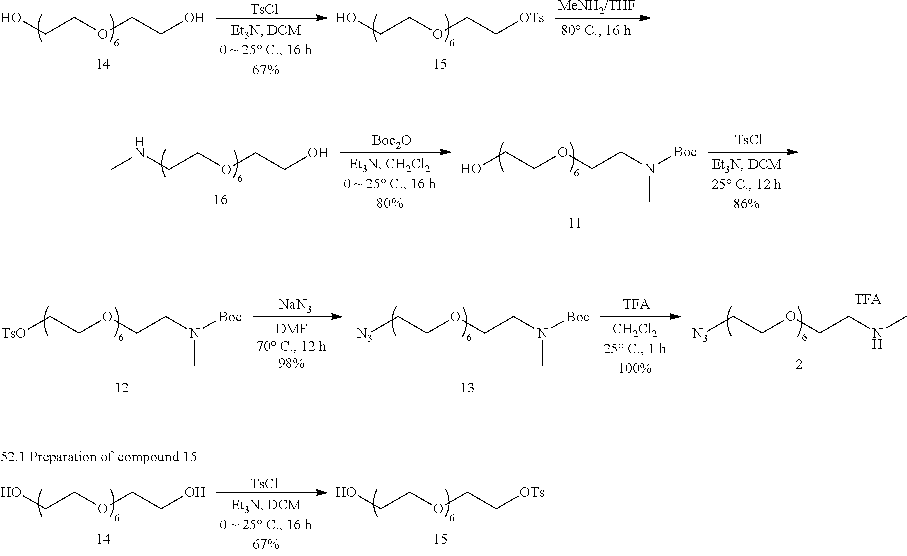

Prepared similarly to compound 1C in Compound I-49. The residue was purified by column chromatography on silica gel (Dichloromethane/Methanol=100/1 to 20/1) to afford 2-1[2-[2-[2-[2-2[2-(2-hydroxyethoxy)ethoxy]ethoxy]ethoxy]ethoxy]ethoxy]ethyl 4-methylbenzenesulfonate (2.86 g, 5.95 mmol, 67% yield) as a colorless oil. ¹H NMR (400 MHz, CDCl₃) δ7.83-7.76 (m, 2H), 7.38-7.31 (m, 2H), 4.19-4.13 (m, 2H), 3.74-3.55 (m, 27H), 2.45 (s, 3H).

### 52.2 Preparation of Compound 16

A mixture of 2-[2-[2-[2-[2-[2-(2-hydroxyethoxy)ethoxy]ethoxy]ethoxy]ethoxy]ethoxy]ethyl 4-methylbenzenesulfonate (3.70 g, 7.70 mmol, 1.0 equiv) in MeNH₂ (7.25 g, 77.0 mmol, 33% purity in THF, 10.0 equiv) was heated to 80° C. and stirred for 16 h. The reaction mixture was concentrated in vacuo to afford 2-[[2[2-[2-[2-[2-(methylamino)ethoxy]ethoxy]ethoxy]ethoxy]ethoxy]ethoxy]ethanol (2.61 g, crude) as a yellow oil. ¹H NMR (400 MHz, CDCl₃) δ3.77-3.71 (m, 4H), 3.65-3.56 (m, 24H), 3.13-3.05 (m, 2H), 2.65 (s, 3H).

### 52.3 Preparation of Compound 11

Prepared similarly to compound 1D in Compound I-49. The residue was purified by column chromatography on silica gel (Dichloromethane/Methanol=100/1 to 10/1) to afford tert-butyl N-[21[2-[2-[12-[2-(2-hydroxyethoxy)ethoxy]ethoxy]ethoxy]ethoxy]ethoxy]ethyl]-N-methyl-carbamate (2.71 g, 6.17 mmol, 80% yield) as a yellow oil. ¹H NMR (400 MHz, CDCl₃) δ3.76-3.53 (m, 26H), 3.43-3.34 (m, 2H), 3.17-2.94 (m, 1H), 2.91 (s, 3H), 1.45 (s, 9H).

### 52.4 Preparation of Compound 12

A mixture of tert-butyl N-[2-[2-[2-[2-[2-[2-(2-hydroxyethoxy)ethoxy]ethoxy]ethoxy]ethoxy]ethoxy]ethyl]-N-methyl-carbamate (1.08 g, 2.46 mmol, 1.0 equiv), Et₃N (746 mg, 7.37 mmol, 3.0 equiv) and TsCl (937 mg, 4.91 mmol, 2.0 equiv) in DCM (15 mL) was stirred at 25° C. for 12 h. To the reaction mixture was added water (30 mL) and the mixture was extracted with DCM (25 mL*2). The combined organic phase was washed with brine (50 mL), dried over anhydrous Na₂SO₄, filtered and concentrated in vacuo. The residue was purified by column chromatography on silica gel (Dichloromethane/Methanol=100/1 to 15/1) to afford 2-[21[2-1[2-1[2-1[2-2[tert-butoxycarbonyl(methyl)amino]ethoxy]ethoxy]ethoxy]ethoxy]ethoxy]ethoxy]ethyl 4-methylbenzene sulfonate (1.25 g, 2.11 mmol, 86% yield) as a colorless oil. ¹H NMR (400 MHz, CDCl₃) δ7.82 (d, 2H, J=8.4 Hz), 7.36 (d, 2H, J=8.4 Hz), 4.20-4.16 (m, 2H), 3.73-3.69 (m, 2H), 3.68-3.63 (m, 18H), 3.45-3.38 (m, 2H), 2.94-2.92 (m, 3H), 2.47 (s, 3H), 1.47 (s, 9H).

### 52.5 Preparation of Compound 13

Prepared similarly to compound 1B in Compound I-49 to afford tert-butyl N-[2-[2-[2-[2-[2-[2-(2-azidoethoxy)ethoxy]ethoxy]-ethoxy]ethoxy]ethoxy]ethyl]-N-methyl-carbamate (960 mg, 2.07 mmol, 98% yield) as a brown oil. ¹H NMR (400 MHz, CDCl₃) δ3.74-3.56 (m, 24H), 3.47-3.35 (m, 4H), 2.93 (s, 3H), 1.47 (s, 9H).

### 52.6 Preparation of Compound 2

Prepared similarly to compound 9 in Compound I-44. The reaction mixture was concentrated in vacuo to afford 2-[2-[2-[2-[2-[2-(2-azidoethoxy)ethoxy]ethoxy]ethoxy]ethoxy]ethoxy]-N-methyl-ethanamine (617 mg, 1.29 mmol, 100% yield, TFA salt) as a yellow oil.

### 52.7 Preparation of Compound 3

A mixture of tert-butyl 4-(7-bromo-2,6-dichloro-8-fluoro-quinazolin-4-yl)piperazine-1-carboxylate (500 mg, 1.04 mmol, 1.0 equiv), 2-[2-[2-[2-[2-[2-(2-azidoethoxy)ethoxy]ethoxy]ethoxy]ethoxy]ethoxy]-N-methyl-ethanamine (598 mg, 1.25 mmol, 1.2 equiv, TFA salt) and DIPEA (1.35 g, 10.4 mmol, 10 equiv) in i-PrOH (3 mL) in a sealed tube was stirred at 95° C. for 12 h. The reaction mixture was diluted with water (20 mL) and extracted with EtOAc (10 mL*2). The combined organic phase was washed with brine (10 mL), dried over anhydrous Na₂SO₄, filtered and concentrated in vacuo. The residue was purified by column chromatography on silica gel (Petroleum ether/Ethyl acetate=5/1 to 1/1) to afford tert-butyl4-[2-[2-[2-[2-[2-[2-[2-(2-azidoethoxy)ethoxy]ethoxy]ethoxy]ethoxy]ethoxy]ethyl-methyl-amino]-7-bromo-6-chloro-8-fluoro-quinazolin-4-yl]piperazine-1-carboxylate (650 mg, 86% purity) as a yellow oil. ¹H NMR (400 MHz, CDCl₃) δ7.57 (s, 1H), 3.93-3.83 (m, 2H), 3.80-3.57 (m, 33H), 3.44-3.38 (m, 2H), 3.31 (s, 3H), 1.51 (s, 9H). LC-MS: MS (ES′): RT=0.932 min, m/z=807.0 [M+H⁺].

### 52.8 Preparation of Compound 5

Prepared similarly to compound 5 in Compound I-51 to afford tert-butyl 4-[2-[2-[2-[2-[2-[2-[2-(2-azidoethoxy)ethoxy]ethoxy]ethoxy]ethoxy]ethoxy]ethyl-methyl-amino]-6-chloro-8-fluoro-7-(3-hydroxy-1-naphthyl)quinazolin-4-yl]piperazine-1-carboxylate (1.35 g, 44% purity) as a brown oil. LC-MS: MS (ES′): RT=0.931 min, m/z=871.3 [M+H⁺].

### 52.9 Preparation of Compound 6

Prepared similarly to compound 6 in Compound I-51. The residue was purified by column chromatography on silica gel (Dichloromethane/Methanol=20/1 to 5/1) to afford tert-butyl 4-[21[2-[2-[2-[2-[2-(2-aminoethoxy)ethoxy]ethoxy]ethoxy]ethoxy]ethoxy]ethyl-methyl-amino]-6-chloro-8-fluoro-7-(3-hydroxy-1-naphthyl)quinazolin-4-yl]piperazine-1-carboxylate (280 mg, 308 μmol, 44% yield) as a yellow oil. ¹H NMR (400 MHz, CDCl₃) δ 7.78-7.73 (m, 1H), 7.64 (s, 1H), 7.45-7.36 (m, 2H), 7.26-7.17 (m, 2H), 7.15-7.11 (m, 1H), 3.78-3.64 (m, 14H), 3.62-3.53 (m, 22H), 3.30 (s, 3H), 2.95 (t, 2H J=4.8 Hz), 1.53 (s, 9H). LC-MS: MS (ES′): RT=0.845 min, m/z=845.4 [M+H⁺].

### 52.10 Preparation of Compound 8

Prepared similarly to compound 5 in Compound I-49. The residue was purified by prep-HPLC (column: Phenomenex Gemini-NX C18 75*30 mm*3 um; mobile phase: [water(0.225% FA)-ACN]; B %: 42%-72%, 7 min) to afford tert-butyl4-[6-chloro-2-[2-[2-[2-[2-[2-[2-[2-[[2-[7-(4-chlorophenyl)-4,5,13-trimethyl-3-thia-1,8,11,12-tetrazatricyclo [8.3.0.0^{2,6}]trideca-2(6), 4,7,10,12-pentaen-9-yl]acetyl]amino]ethoxy]ethoxy]ethoxy]ethoxy]ethoxy]ethoxy]ethyl-methyl-amino]-8-fluoro-7-(3-hydroxy-1-naphthyl)quinazolin-4-yl]piperazine-1-carboxylate (160 mg, crude) as a yellow solid. ¹H NMR (400 MHz, CDCl₃) δ7.75-7.63 (m, 2H), 7.50-7.29 (m, 8H), 7.25-7.16 (m, 2H), 4.78-4.68 (m, 1H), 3.86-3.37 (m, 40H), 3.36-3.30 (m, 3H), 2.71 (s, 3H), 2.41 (s, 3H), 1.68-1.66 (m, 3H), 1.53 (s, 9H). LC-MS: MS (ES⁺): RT=0.892 min, m/z=1229.2 [M+3H⁺].

### 52.11 Preparation of Compound 9

A mixture of tert-butyl 4-[6-chloro-2-[2-[2-[2-[2-[2-[2-[2-[[2-[7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetyl]amino]ethoxy]ethoxy]ethoxy]ethoxy]ethoxy]ethoxy]ethyl-methyl-amino]-8-fluoro-7-(3-hydroxy-1-naphthyl)quinazolin-4-yl]piperazine-1-carboxylate (160 mg, 130 μmol, 1.0 equiv) in DCM (3 mL) and TFA (2 mL) was stirred at 20° C. for 2 h. The reaction mixture was concentrated in vacuo to afford N-[2-[2-[2-[2-[2-[2-[2-[[6-chloro-8-fluoro-7-(3-hydroxy-1-naphthyl)-4-piperazin-1-yl-quinazolin-2-yl]-methyl-amino]ethoxy]ethoxy]ethoxy]ethoxy]ethoxy]ethoxy]ethyl]-2-[7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetamide (161 mg, crude, TFA salt) as a yellow oil. LC-MS: MS (ES⁺): RT=0.774 min, m/z=1127.2 [M+H⁺].

### 52.12 Preparation of Compound I-52

To a mixture of N-[2-[2-[2-[2-[2-[2-[2-[[6-chloro-8-fluoro-7-(3-hydroxy-1-naphthyl)-4-piperazin-1-yl-quinazolin-2-yl]-methyl-amino]ethoxy]ethoxy]ethoxy]ethoxy]ethoxy]ethoxy]ethyl]-2-[7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetamide (160 mg, 129 μmol, 1.0 equiv, TFA salt) and NaHCO₃ (108 mg, 1.29 mmol, 10.0 equiv) in THF (5 mL) and H₂O (1 mL) was added a solution of prop-2-enoyl chloride (11.7 mg, 129 μmol, 1.0 equiv) in THF (0.5 mL) at 0° C. The reaction mixture was stirred at 0° C. for 10 min. The reaction mixture was diluted with water (5 mL) and extracted with ethyl acetate (5 mL*3). The combined organic phase was washed with brine (5 mL), dried with anhydrous Na₂SO₄, filtered and concentrated in vacuo. The residue was purified by prep-HPLC (column: Phenomenex Gemini-NX C18 75*30 mm*3 um; mobile phase: [water(0.225% FA)-ACN]; B %: 32%-62%, 7 min) to afford N-[2-[22[2-[2-[2-[2-[[6-chloro-8-fluoro-7-(3-hydroxy-1-naphthyl)-4-(4-prop-2-enoylpiperazin-1-yl)quinazolin-2-yl]-methyl-amino]ethoxy]ethoxy]ethoxy]ethoxy]ethoxy]ethoxy]ethyl]-2-[7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetamide (74.0 mg, 62.6 μmol, 49% yield, 100% purity) as a white solid. ¹HNMR (400 MHz, CDCl3) δ7.74-7.60 (m, 2H), 7.48-7.28 (m, 8H), 7.18 (s, 2H), 6.71-6.50 (m, 1H), 6.343-6.33 (m, 1H), 5.81-5.74 (m, 1H), 4.77-4.66 (m, 1H), 3.98-3.41 (m, 40H), 3.30 (s, 3H), 2.68 (s, 3H), 2.39 (s, 3H), 1.66 (d, J=5.6 Hz, 3H). LC-MS: MS (ES⁺): RT=1.604 min, m/z=1181.3 [M+H⁺]; LC-MS method 25.

### Example 53—Synthesis of Compound I-53

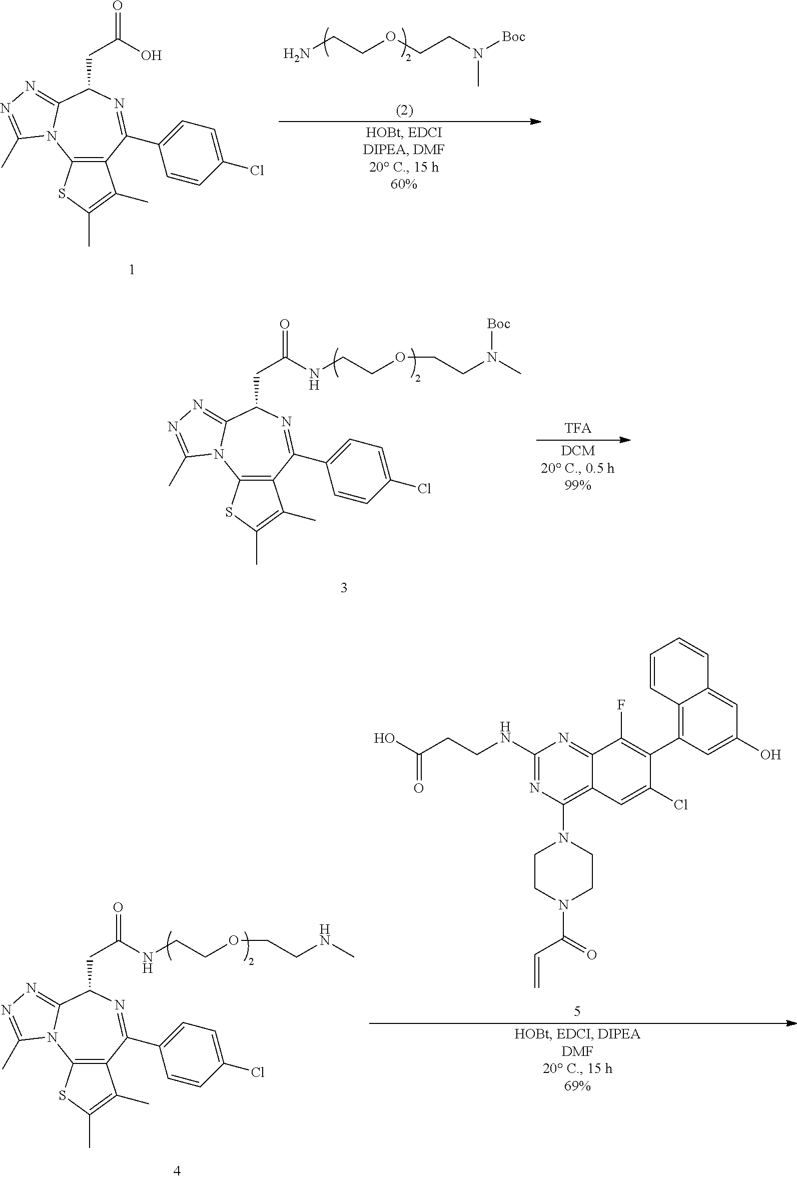

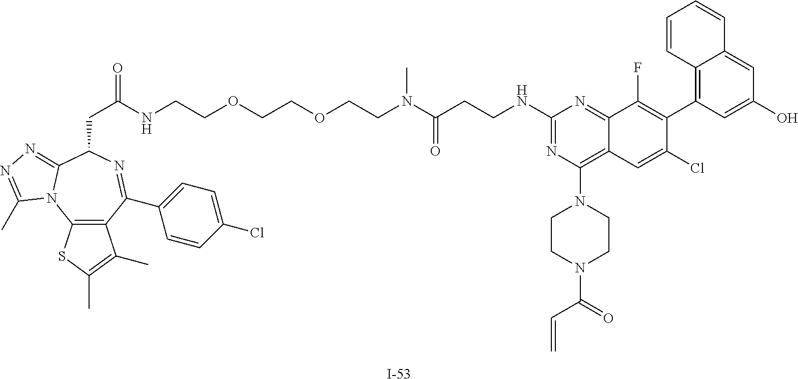

### 53.1 Preparation of Compound 3

A mixture of 2-[(9S)-7-(4-chlorophenyl)-4,5,13-trimethyl-3-thia-1,8,11,12-tetrazatricyclo[8.3.0.02,6]trideca-2(6), 4,7,10,12-pentaen-9-yl]acetic acid (131 mg, 327 μmol, 1.0 equiv), tert-butyl N-[2-[2-(2-aminoethoxy)ethoxy]ethyl]-N-methyl-carbamate (94.3 mg, 360 μmol, 1.1 equiv), HOBt (88.3 mg, 654 μmol, 2.0 equiv), EDCI (125 mg, 654 μmol, 2.0 equiv) and DIPEA (253 mg, 1.96 mmol, 342 μL, 6.0 equiv) in DMF (2 mL) was stirred at 20° C. for 15 h. The reaction mixture was diluted with water (5 mL) and extracted with ethyl acetate (5 mL*3). The combined organic phase was washed with brine (5 mL), dried with anhydrous Na₂SO₄, filtered and concentrated in vacuo. The residue was purified by prep-TLC on silica gel (DCM/MeOH=10/1) to afford tert-butyl N-[2-[2-[2-[[2-[(9S)-7-(4-chlorophenyl)-4,5,13-trimethyl-3-thia-1,8,11,12-tetrazatricyclo [8.3.0.02,6]trideca-2(6), 4,7,10,12-pentaen-9-yl]acetyl]amino]ethoxy]ethoxy]ethyl]-N-methyl-carbamate (130 mg, 195 μmol, 60% yield) as a yellow solid. ¹H NMR (400 MHz, CDCl₃) δ7.46-7.42 (m, 2H), 7.37-7.34 (m, 2H), 6.88 (brs, 1H), 4.73-4.71 (m, 1H), 3.68-3.56 (m, 10H), 3.53-3.49 (m, 2H), 3.47-3.40 (m, 3H), 2.92 (s, 3H), 2.76 (s, 3H), 2.43 (s, 3H), 1.69 (s, 3H), 1.46 (s, 9H). LC-MS: MS (ES⁺): RT=0.932 min, m/z=645.1 [M+H⁺].

### 53.2 Preparation of Compound 4

Prepared similarly to compound 9 in Compound I-44. The reaction mixture was concentrated in vacuo to afford 2-[(9S)-7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]-N-[2-[2-[2-(methylamino) ethoxy]ethoxy]ethyl]acetamide (43.5 mg, 66.0 μmol, 99% yield, TFA salt) as a yellow oil. LC-MS: MS (ES⁺): RT=0.558 min, m/z=545.1 [M+H⁺].

### 53.3 Preparation of Compound I-53

A mixture of 2-[(9S)-7-(4-chlorophenyl)-4,5,13-trimethyl-3-thia-1,8,11,12-tetrazatricyclo [8.3.0.0^{2,6}]trideca-2(6), 4,7,10,12-pentaen-9-yl]-N-[2-[2-[2-(methylamino) ethoxy]ethoxy]ethyl]acetamide (43.1 mg, 65.5 μmol, 1.2 equiv, TFA salt), 3-[[6-chloro-8-fluoro-7-(3-hydroxy-1-naphthyl)-4-(4-prop-2-enoylpiperazin-1-yl)quinazolin-2-yl]amino]propanoic acid (30.0 mg, 54.6 μmol, 1.0 equiv), HOBt (14.7 mg, 109 μmol, 2.0 equiv), EDCI (20.9 mg, 109 μmol, 2.0 equiv) and DIPEA (56.4 mg, 436 μmol, 76.0 μL, 8.0 equiv) in DMF (1 mL) was stirred at 20° C. for 15 h. The reaction mixture was diluted with water (5 mL) and the mixture was extracted with ethyl acetate (5 mL*3). The combined organic phase was washed with brine (5 mL), dried with anhydrous Na₂SO₄, filtered and concentrated in vacuo. The residue was purified by prep-HPLC (column: Unisil 3-100 C18 Ultra 150*50 mm*3 um; mobile phase: [water(0.225% FA)-ACN]; B %: 26%-56%, 10 min) to afford 3-[[6-chloro-8-fluoro-7-(3-hydroxy-1-naphthyl)-4-(4-prop-2-enoylpiperazin-1-yl)quinazolin-2-yl]amino]-N-[2-[2-[2-[[2-[(9S)-7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetyl]amino]ethoxy]ethoxy]ethyl]-N-methyl-propanamide (45.0 mg, 37.6 μmol, 69% yield, 90% purity) as a yellow solid. ¹H NMR (400 MHz, CDCl₃) δ8.97-8.86 (m, 1H), 7.80-7.75 (m, 1H), 7.68-7.60 (m, 2H), 7.44-7.28 (m, 6H), 7.25-7.14 (m, 3H), 6.57-6.50 (m, 1H), 6.37-6.33 (m, 1H), 5.77-5.74 (m, 1H), 4.78-4.74 (m, 1H), 4.27-4.13 (m, 4H), 3.93-3.79 (m, 6H), 3.65-3.43 (m, 15H), 2.99-2.93 (m, 3H), 2.72-2.66 (m, 4H), 2.38 (s, 3H), 1.65 (s, 3H). LC-MS: MS (ES⁺): RT=1.505 min, m/z=1077.3 [M+H⁺]; LC-MS method 40.

### Example 54—Synthesis of Compound I-54

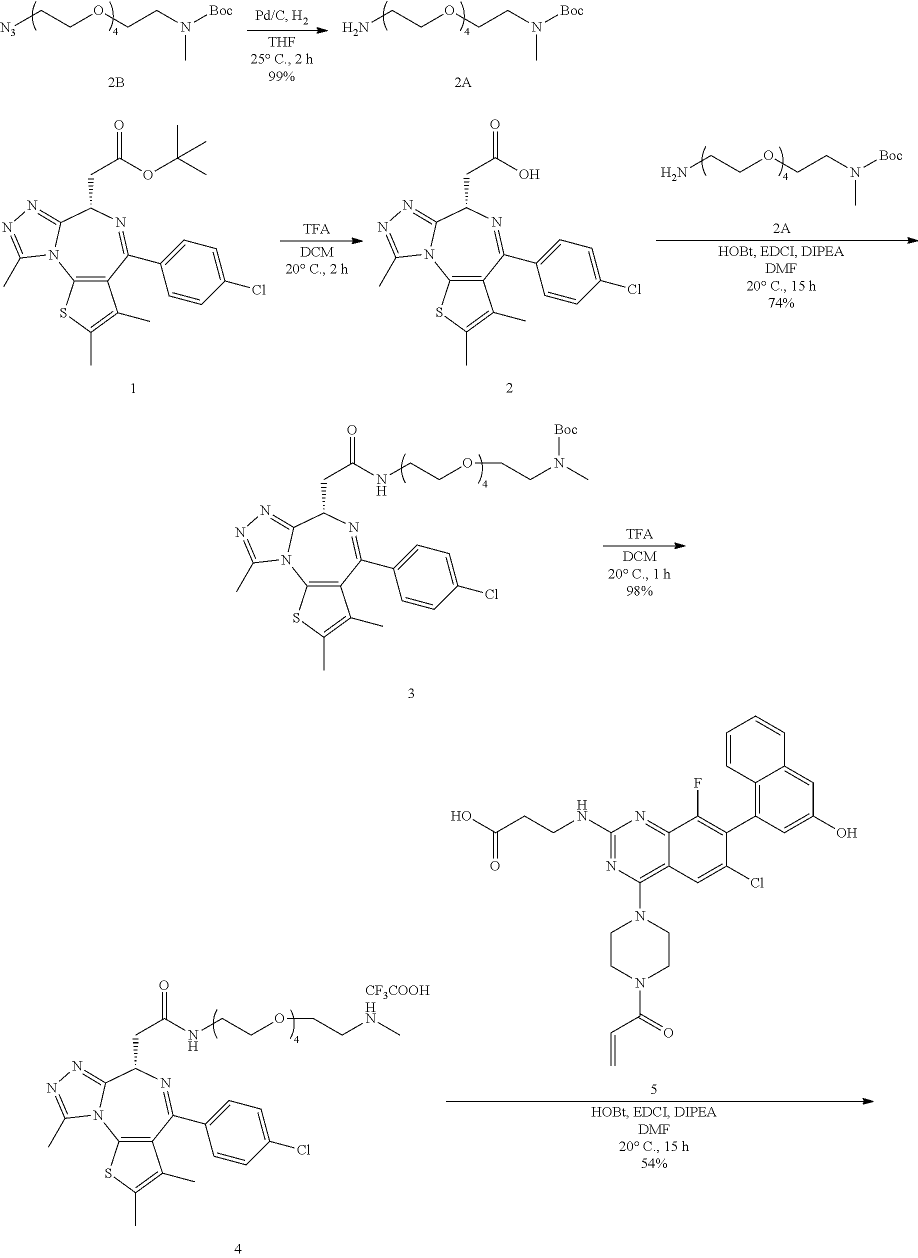

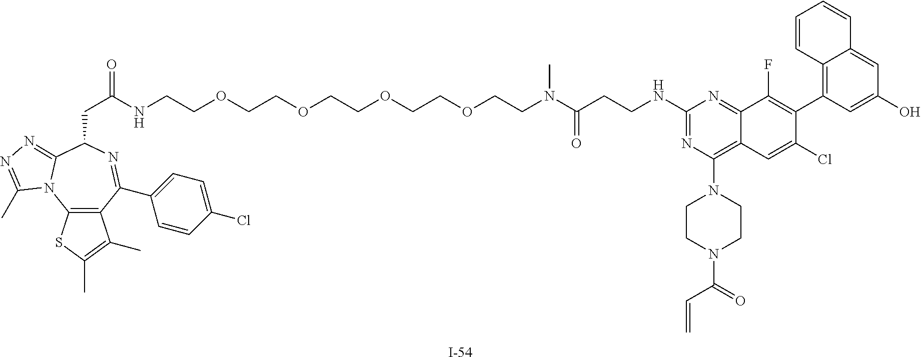

### 54.1 Preparation of Compound 2A

To a solution of tert-butyl N-[2-[2-[2-[2-(2-azidoethoxy)ethoxy]ethoxy]ethoxy]ethyl]-N-methyl-carbamate (130 mg, 0.34 mmol, 1.0 equiv) in THF (10 mL) was added wet Pd/C (20 mg, 10% purity). The mixture was stirred at 25° C. for 2 h under H₂ (15 psi). The reaction mixture was filtered and the filtrate was concentrated in vacuo to afford tert-butyl N-[2-[2-[2-[2-(2-aminoethoxy)ethoxy]ethoxy]ethoxy]ethyl]-N-methyl-carbamate (120 mg, 0.34 mmol, 99% yield) as a yellow oil. ¹H NMR (400 MHz, DMSO-d₆): δ3.51-3.43 (m, 14H), 3.35-3.27 (m, 6H), 2.81-2.78 (m, 3H), 1.38 (s, 9H).

### 54.2 the Preparation of Compound 2

A mixture of tert-butyl 2-[7-(4-chlorophenyl)-4,5,13-trimethyl-3-thia-1,8,11,12-tetrazatricyclo[8.3.0.02,6]trideca-2(6), 4,7,10,12-pentaen-9-yl]acetate (150 mg, 0.33 mmol, 1.0 equiv) in DCM (2 mL) and TFA (1 mL) was stirred at 20° C. for 2 h. The reaction mixture was concentrated in vacuo to afford 2-[7-(4-chlorophenyl)-4,5,13-trimethyl-3-thia-1,8,11,12-tetrazatricyclo[8.3.0.02,6]trideca-2(6), 4,7,10,12-pentaen-9-yl]acetic acid (132 mg, crude) as a yellow oil. LC-MS: MS (ES⁺): RT=0.630 min, m/z=401.0 [M+H⁺].

### 54.3 the Preparation of Compound 3

Prepared similarly to compound 3 in Compound I-53. The residue was purified by prep-TLC on silica gel (Dichloromethane:Methanol=10:1) to afford tert-butyl N-[2-[2-[2-[2-[2-[[2-[7-(4-chlorophenyl)-4,5,13-trimethyl-3-thia-1,8,11,12-tetrazatricyclo [8.3.0.02,6]trideca-2(6), 4,7,10,12-pentaen-9-yl]acetyl]amino]ethoxy]ethoxy]ethoxy]ethoxy]ethyl]-N-methylcarbamate (180 mg, 0.24 mmol, 74% yield) as a yellow oil. ¹H NMR (400 MHz, CDCl₃) δ7.45-7.43 (m, 2H), 7.37-7.34 (m, 2H), 7.01-7.00 (m, 1H), 4.74 (t, 1H, J=6.8 Hz), 3.69-3.68 (m, 7H), 3.66-3.58 (m, 9H), 3.41-3.38 (m, 2H), 2.98-2.94 (m, 5H), 2.91-2.88 (m, 5H), 2.43 (s, 3H), 1.69 (s, 3H), 1.45 (s, 9H). LC-MS: MS (ES⁺): RT=0.727 min, m/z=733.1 [M+H⁺].

### 54.4 the Preparation of Compound 4

Prepared similarly to compound 9 in Compound I-44. The reaction mixture was concentrated in vacuo to afford 2-[7-(4-chlorophenyl)-4,5,13-trimethyl-3-thia-1,8,11,12-tetrazatricyclo[8.3.0.02,6]trideca-2(6), 4,7,10,12-pentaen-9-yl]-N-[2-[2-[2-[2-[2-(methylamino)ethoxy]ethoxy]ethoxy]ethoxy]ethyl]acetamide (75 mg, 0.10 mmol, 98% yield, TFA salt) as a yellow oil. LC-MS: MS (ES⁺): RT=0.918 min, m/z=633.2 [M+H⁺].

### 54.5 the Preparation of Compound I-54

A mixture of 3-[[6-chloro-8-fluoro-7-(3-hydroxy-1-naphthyl)-4-(4-prop-2-enoylpiperazin-1-yl)quinazolin-2-yl]amino]propanoic acid (40 mg, 72.7 μmol, 1.0 equiv), 2-[7-(4-chlorophenyl)-4,5,13-trimethyl-3-thia-1,8,11,12-tetrazatricyclo[8.3.0.02,6]trideca-2(6), 4,7,10,12-pentaen-9-yl]-N-[2-[2-[2-[2-[2-(methylamino)ethoxy]ethoxy]ethoxy]ethoxy]ethyl]acetamide (65.2 mg, 87.3 μmol, 1.2 equiv, TFA salt), HOBt (24.6 mg, 182 μmol, 2.5 equiv), EDCI (34.9 mg, 182 μmol, 2.5 equiv) and DIPEA (47.0 mg, 364 μmol, 5.0 equiv) in DMF (2 mL) was stirred at 20° C. for 15 h under N₂. The reaction mixture was diluted with water (5 mL) and extracted with ethyl acetate (10 mL*3). The combined organic phase was washed with brine (15 mL), dried over anhydrous Na₂SO₄, filtered and concentrated in vacuo. The residue was purified by prep-HPLC (FA condition:column: Phenomenex Luna C18 150*25 mm*l0 um; mobile phase: [water(0.225% FA)-ACN]; B %: 24%-54%, 10 min) to afford 3-[[6-chloro-8-fluoro-7-(3-hydroxy-1-naphthyl)-4-(4-prop-2-enoylpiperazin-1-yl)quinazolin-2-yl]amino]-N-[2-[2-[2-[2-[2-[[2-1[7-(4-chlorophenyl)-4,5,13-trimethyl-3-thia-1,8,11,12-tetrazatricyclo1[8.3.0.02,6]trideca-2(6), 4,7,10,12-pentaen-9-yl]acetyl]amino]ethoxy]ethoxy]ethoxy]ethoxy]ethyl]-N-methyl-propanamide (46.4 mg, 39.2 μmol, 54% yield, 98.6% purity) as a off-white solid. ¹H NMR (400 MHz, CDCl₃) δ7.74-7.67 (m, 2H), 7.43-7.38 (m, 8H), 7.22-7.20 (m, 1H), 6.67-6.58 (m, 1H), 6.40-6.36 (m, 1H), 5.80-5.78 (m, 1H), 4.76-4.70 (m, 1H), 3.95-3.80 (m, 8H), 3.63-3.43 (m, 23H), 3.11-3.08 (m, 1H), 2.97-2.93 (m, 2H), 2.86-2.76 (m, 1H), 2.70 (s, 3H), 2.40 (s, 3H), 1.71-1.66 (m, 8H). LC-MS: MS (ES′): RT=1.570 min, m/z=1188.3 [M+Na⁺]; LC-MS method 40.

### Example 55—Synthesis of Compound I-55

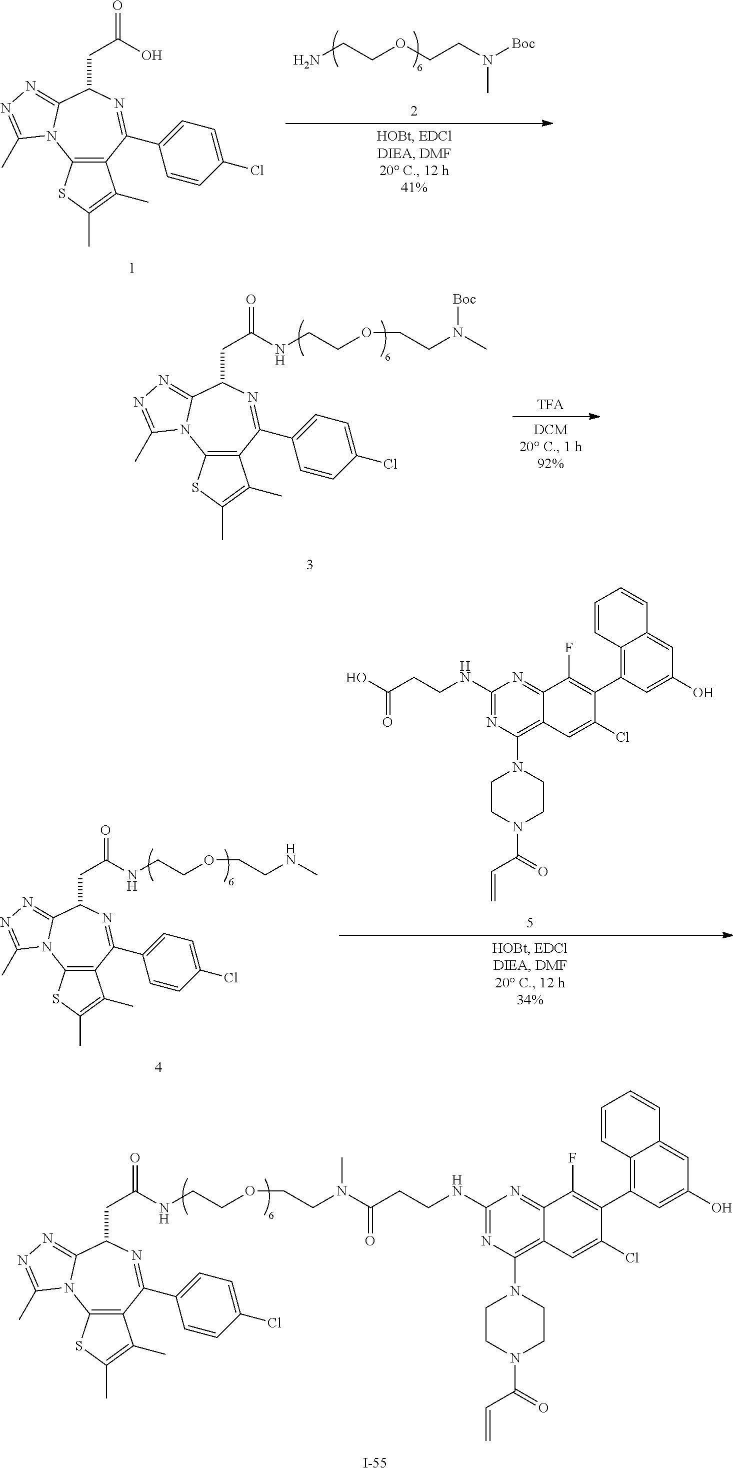

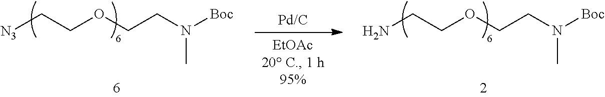

### 55.1 Preparation of Compound 2

To a solution of tert-butyl N-[2-[2-[2-[2-[2-[2-(2-azidoethoxy)ethoxy]ethoxy]ethoxy]ethoxy]ethoxy]ethyl]-N-methyl-carbamate (200 mg, 431 μmol, 1.0 equiv) in THF (8 mL) was added Pd/C (40.0 mg, 10% purity) under N₂. The mixture was degassed and stirred at 20° C. for 1 h under H₂ (15 psi). The reaction mixture was filtered and the filtrate was concentrated in vacuo to afford tert-butyl N-[2-[2-[2-[2-[2-[2-(2-aminoethoxy)ethoxy]ethoxy]ethoxy]ethoxy]ethoxy]ethyl]-N-methyl-carbamate (180 mg, 410 μmol, 95% yield) as a yellow oil. ¹H NMR (400 MHz, CDCl₃) δ3.92-3.86 (m, 1H), 3.75-3.48 (m, 25H), 3.35-3.29 (m, 2H), 2.84 (s, 3H), 1.38 (s, 9H).

### 55.2 Preparation of Compound 3

A mixture of 2-[7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetic acid (130 mg, 324 μmol, 1.0 equiv), tert-butyl N-[2-[2-[2-[2-[2-[2-(2-aminoethoxy)ethoxy]ethoxy]ethoxy]ethoxy]ethoxy]ethyl]-N-methyl-carbamate (178 mg, 405 μmol, 1.25 equiv), HOBt (87.6 mg, 648 μmol, 2.0 equiv), EDCI (249 mg, 1.30 mmol, 4.0 equiv) and DIEA (251 mg, 1.95 mmol, 6.0 equiv) in DMF (8 mL) was stirred at 20° C. for 12 h under N₂. The reaction mixture was diluted with water (20 mL) and the mixture was extracted with EtOAc (10 mL*2), the combined organic was washed with brine (10 mL*3), dried over anhydrous Na₂SO₄, the mixture was filtered and the filtrate was concentrated in vacuo. The residue was purified by preparative TLC on silica gel (Dichloromethane/Methanol=10/1) to afford tert-butyl N-[2-[2-[2-[2-[2-[2-[2-[[2-[(9S)-7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetyl]amino]ethoxy]ethoxy]ethoxy]ethoxy]ethoxy]ethoxy]ethyl]-N-methyl-carbamate (110 mg, 134 μmol, 41% yield) as a yellow oil. ¹H NMR (400 MHz, CDCl₃) δ7.57-7.32 (m, 5H), 5.05-4.87 (m, 1H), 3.79-3.46 (m, 29H), 3.44-3.36 (m, 2H), 2.97-2.94 (m, 2H), 2.94-2.92 (m, 3H), 2.52-2.45 (m, 3H), 1.76-1.66 (m, 3H), 1.47 (s, 9H).

### 55.3 Preparation of Compound 5

Prepared similarly to compound 9 in Compound I-44. The reaction mixture was concentrated in vacuo to afford 2-[(9S)-7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]-N-[2-[2-[2-[2-[2-[2-[2-(methylamino)ethoxy]ethoxy]ethoxy]ethoxy]ethoxy]ethoxy]ethyl]acetamide (55.0 mg, 61.9 mol, 92% yield, TFA salt) as a yellow oil. LC-MS: MS (ES⁺): RT=0.760 min, m/z=721.1 [M+H⁺].

### 55.4 Preparation of Compound I-55

A mixture of 2-[7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]-N-[2-[2-[2-[2-[2-[2-[2-(methylamino)ethoxy]ethoxy]ethoxy]ethoxy]ethoxy]ethoxy]ethyl]acetamide (54.7 mg, 65.5 mol, 1.2 equiv, TFA salt), 3-[[6-chloro-8-fluoro-7-(3-hydroxy-1-naphthyl)-4-(4-prop-2-enoylpiperazin-1-yl)quinazolin-2-yl]amino]propanoic acid (30.0 mg, 54.6 μmol, 1.0 equiv), HOBt (14.7 mg, 109 μmol, 2.0 equiv), EDCI (31.4 mg, 164 μmol, 3.0 equiv) and DIEA (42.3 mg, 327 μmol, 6.0 equiv) in DMF (2 mL) was stirred at 20° C. for 12 h under N₂ atmosphere. The reaction mixture was filtered. The filtrate was diluted with water (20 mL) and the mixture was extracted with EtOAc (10 mL*2). The combined organic was washed with brine (10 mL *3), dried over anhydrous Na₂SO₄, filtered and concentrated in vacuo. The residue was purified by prep-HPLC (column: Phenomenex Gemini-NX C18 75*30 mm*3 um; mobile phase: [water(0.225% FA)-ACN]; B %: 28%-58%, 7 min) to afford 3-[[6-chloro-8-fluoro-7-(3-hydroxy-1-naphthyl)-4-(4-prop-2-enoylpiperazin-1-yl)quinazolin-2-yl]amino]-N-[2-[2-[2-[2-[2-[2-[2-[[2-[7-(4-chlorophenyl)-4,5,13-trimethyl-3-thia-1,8,11,12-tetrazatricyclo[8.3.0.0^{2,6}]trideca-2(6), 4,7,10,12-pentaen-9-yl]acetyl]amino]ethoxy]ethoxy]ethoxy]ethoxy]ethoxy]ethoxy]ethyl]-N-methyl-propanamide (24.0 mg, 18.4 μmol, 34% yield, 96% purity) as a white solid. ¹H NMR (400 MHz, CDCl₃) δ7.77-7.63 (m, 2H), 7.48-7.30 (m, 8H), 7.25-7.02 (m, 2H), 6.69-6.57 (m, 1H), 6.44-6.35 (m, 1H), 5.83-5.78 (m, 1H), 4.79-4.68 (m, 1H), 4.00-3.75 (m, 10H), 3.68-3.42 (m, 32H), 3.18-2.94 (m, 3H), 2.86-2.72 (m, 2H), 2.71 (s, 3H), 2.41 (s, 3H), 1.69 (s, 3H). LC-MS: MS (ES⁺): RT=2.326 min, m/z=627.0 [M/2+H⁺]; LC-MS method 25.

### Example 56—Synthesis of Compound I-56

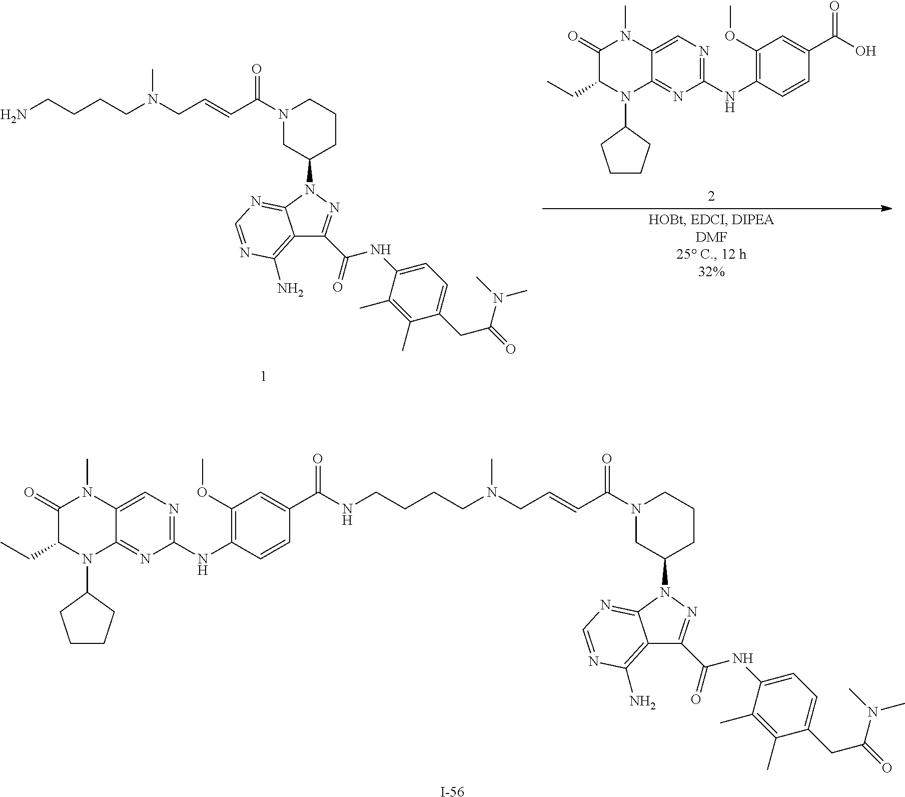

A mixture of 4-amino-[(3R)-1-[(E)-4-[4-aminobutyl(methyl)amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (55.0 mg, 66.8 μmol, 1 equiv), 4-amino-1-[(3R)-1-[(E)-4-[8-aminooctyl(methyl)amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (28.4 mg, 66.8 μmol, 1 equiv, TFA salt), HOBt (13.5 mg, 100 μmol, 1.5 equiv), EDCI (38.4 mg, 200 μmol, 3 equiv) and DIPEA (69.1 mg, 534 μmol, 8 equiv) in DMF (0.6 mL) was stirred at 25° C. for 11 h under N₂. The reaction mixture was diluted with water (2 mL) and extracted with EtOAc (3 mL*5). The combined organic phase was washed with brine (3 mL), dried over anhydrous Na₂SO₄, filtered and concentrated in vacuo. The residue was purified by prep-HPLC (column: Phenomenex Gemini-NX C18 75*30 mm*3 um; mobile phase: [water (0.225% TFA) -ACN]; B%: 12%-42%, 7 min) to afford 4-amino-1-[(3R)-1-[(E)-4-[4-[[4-[(8-cyclopentyl-7-ethyl-5-methyl-6-oxo-7H-pteridin-2-yl)amino]-3-methoxy-benzoyl]amino]butyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (22.0 mg, 21.2 μmol, 32% yield, 99% purity) as a white solid. ¹H NMR (400 MHz, DMSO-d₆): δ10.23-9.95 (m, 1H), 8.68-8.47 (m, 1H), 8.43-8.37 (m, 1H), 8.32-8.02 (m, 3H), 7.85-7.81 (m, 1H), 7.59 (s, 1H), 7.50-7.43 (m, 2H), 7.23-7.08 (m, 1H), 6.95 (d, 1H, J=8.0 Hz), 6.97-6.93 (m, 1H), 6.68-6.43 (m, 2H), 4.35-4.03 (m, 5H), 3.92 (s, 3H), 3.71 (s, 3H), 3.24 (s, 3H), 3.08-2.96 (m, 6H), 2.86 (s, 3H), 2.18-1.35 (m, 31H), 0.76 (t, 3H, J=7.6 Hz). LC-MS: MS (ES′): RT=2.102 min, m/z=1026.5 [M+H⁺]; LC-MS Method 10.

### Example 57—Synthesis of Compound I-57

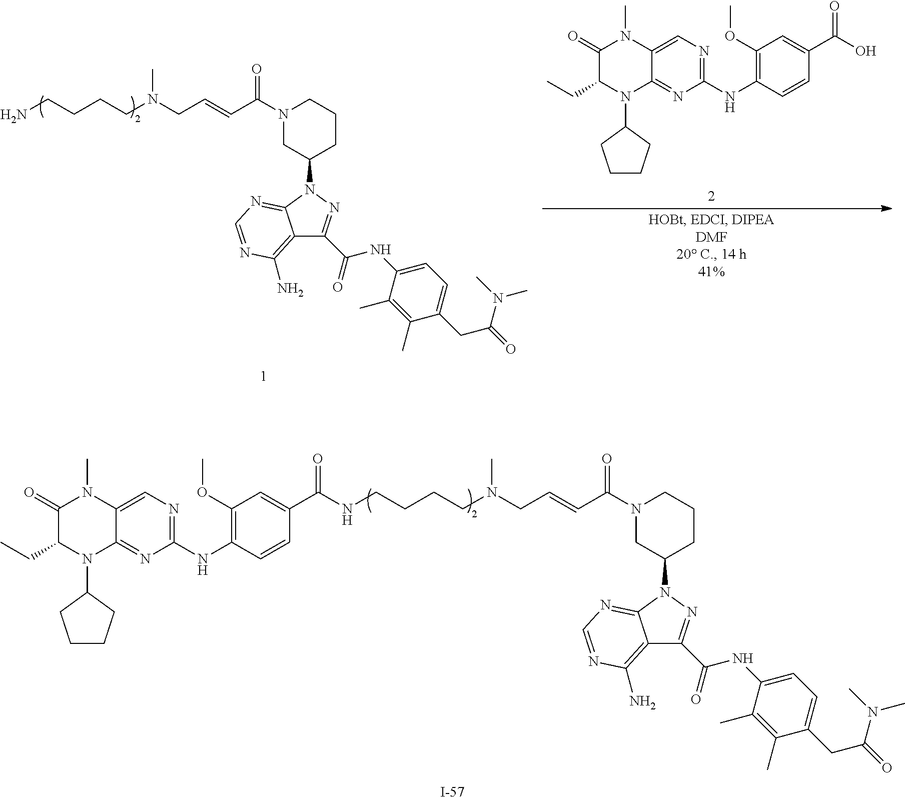

A mixture of 4-[(8-cyclopentyl-7-ethyl-5-methyl-6-oxo-7H-pteridin-2-yl)amino]-3-methoxy-benzoic acid (40.0 mg, 94.0 μmol, 1 equiv), 4-amino-1-[(3R)-1-[(E)-4-[8-aminooctyl(methyl)amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (74.2 mg, 94.0 μmol, 1 equiv, TFA salt), HOBt (31.8 mg, 235 μmol, 2.5 equiv), EDCI (45.1 mg, 235 μmol, 2.5 equiv) and DIPEA (60.8 mg, 470 μmol, 81.9 μL, 5 equiv) in DMF (1 mL) was stirred at 20° C. for 14 h under N₂. The reaction mixture was diluted with water (10 mL) and extracted with DCM/MeOH (10/1, 15 mL*3). The combined organic phase was washed with brine (20 mL), dried over anhydrous Na₂SO₄, filtered and concentrated in vacuo. The residue was purified by prep-HPLC (FA condition:column: Phenomenex luna C18 150*25 mm* 10 um; mobile phase: [water(0.225% FA)-ACN]; B %: 12%-42%, 10 min) to afford 4-amino-[(3R)-1-[(E)-4-[8-[[4-[(8-cyclopentyl-7-ethyl-5-methyl-6-oxo-7H-pteridin-2-yl)-amino]-3-methoxy-benzoyl]amino]octyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (42.2 mg, 38.4 μmol, 41% yield, 98% purity) as a white solid. ¹H NMR (400 MHz, DMSO-d₆): δ10.22-10.20 (m, 1H), 8.85-8.62 (m, 1H), 8.41-8.39 (m, 1H), 8.32-8.29 (m, 1H), 8.26-8.24 (m, 1H), 8.10 (brs, 1H), 7.84 (s, 1H), 7.60 (s, 1H), 7.50-7.46 (m, 2H), 7.22-7.12 (m, 1H), 6.96-6.94 (m, 1H), 6.64-6.63 (m, 1H), 6.53-6.44 (m, 1H), 4.83-4.51 (m, 2H), 4.33 (t, 1H, J=8.4 Hz), 4.25-4.10 (m, 2H), 4.08-4.03 (m, 1H), 3.92 (s, 3H), 3.85-3.78 (m, 1H), 3.73-3.69 (m, 2H), 3.26-3.22 (m, 6H), 3.09-3.08 (m, 1H), 3.05 (s, 3H), 2.97-2.95 (m, 1H), 2.86 (s, 3H), 2.28-2.27 (m, 1H), 2.16-2.10 (m, 10H), 2.03-2.00 (m, 3H), 1.90-1.86 (m, 2H), 1.80-1.70 (m, 4H), 1.65-1.60 (m, 4H), 1.52-1.46 (m, 2H), 1.40-1.38 (m, 1H), 1.30-1.10 (m, 9H), 0.75 (t, 3H, J=7.6 Hz). LC-MS: MS (ES′): RT=1.788 min, m/z=1082.7 [M+H⁺]; LC-MS Method 25.

### Example 58—Synthesis of Compound I-58

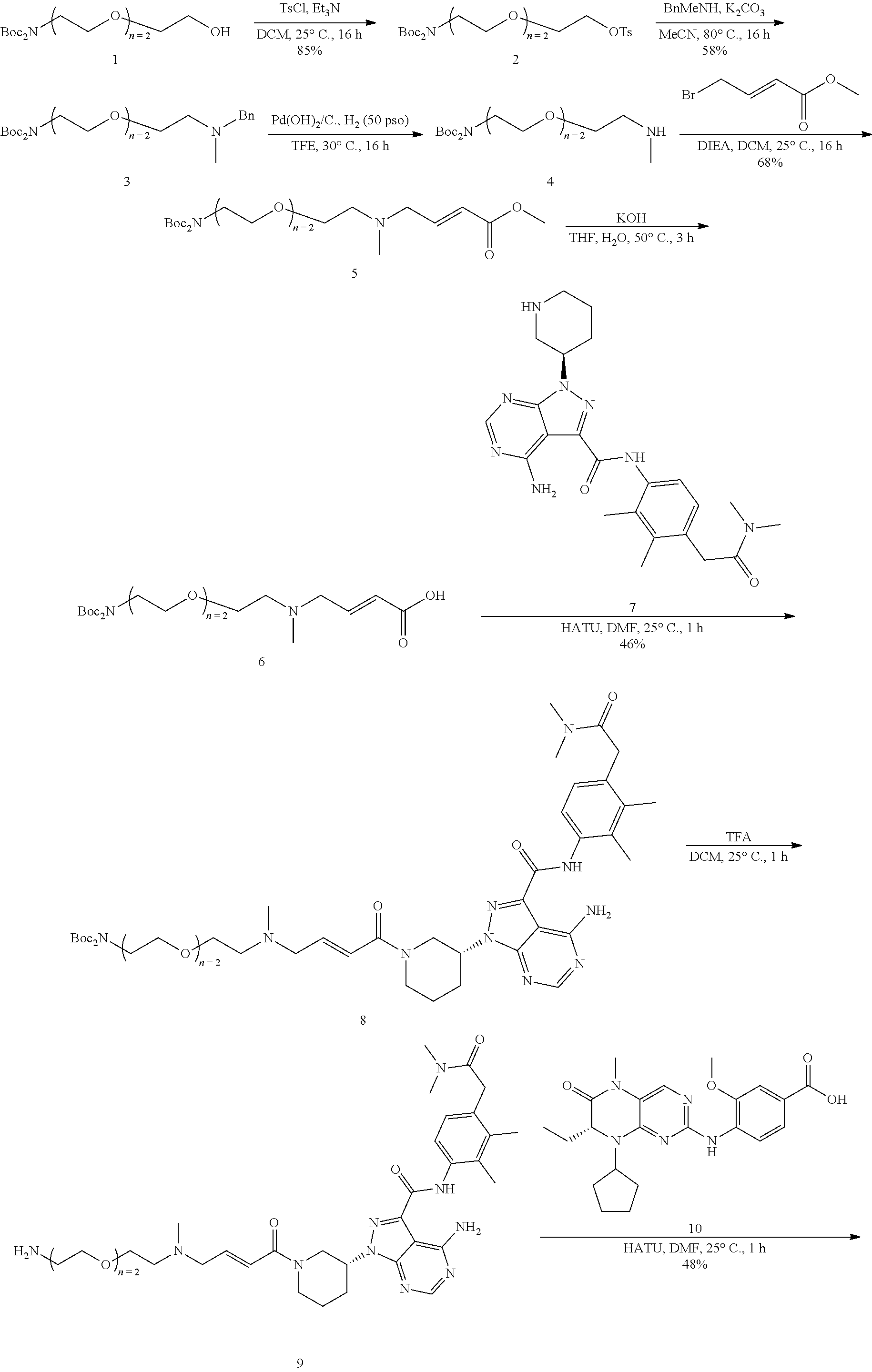

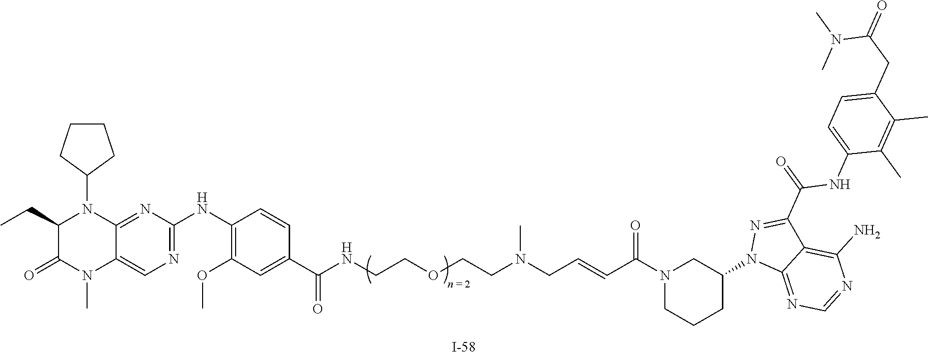

### 58.1 Preparation of Compound 2

To a solution of tert-butyl N-tert-butoxycarbonyl-N-[2-[2-(2-hydroxyethoxy) ethoxy]ethyl]carbamate (10.0 g, 28.6 mmol, 1.0 eq) and TEA (8.69 g, 85.9 mmol, 3.0 eq) in DCM (100 mL) was added 4-methylbenzenesulfonyl chloride (10.9 g, 57.2 mmol, 2.0 eq) at 0° C. The mixture was stirred at 25° C. for 16 h. The reaction mixture was diluted with water (80 mL), extracted with DCM (40 mL*3). The combined organic layers were dried over Na₂SO₄ and filtered. The filtrate was concentrated under to give a residue. The residue was purified by column chromatography (SiO₂, Petroleum ether: Ethyl acetate=20: 1 to 1: 3) to give the desired product 2-[2-[2-[bis(tert-butoxycarbonyl)amino]ethoxy]ethoxy]ethyl-4-methylbenzenesulfonate (12.6 g, 24.3 mmol, 85% yield, 97% purity) as a light yellow oil.

### 58.2 Preparation of Compound 3

To a solution of 2-[2-[2-[bis(tert-butoxycarbonyl)amino]ethoxy]ethoxy]ethyl 4-methylbenzenesulfonate (12.6 g, 25.0 mmol, 1.0 eq) and N-methyl-1-phenyl-methanamine (3.03 g, 25.0 mmol, 1.0 eq) in MeCN (100 mL) was added K₂CO₃ (6.92 g, 50.0 mmol, 2.0 eq). The mixture was stirred at 80° C. for 16 h. The mixture was filtered. The filtrate was concentrated to give a residue. The residue was purified by column chromatography (SiO₂, Petroleum ether: Ethyl acetate=20: 1 to 0: 1) to give the desired product tert-butyl N-[2-[2-[2-[benzyl(methyl) amino]ethoxy]ethoxy]ethyl]-N-tert-butoxycarbonyl-carbamate (6.70 g, 14.5 mmol, 56% yield, 98% purity) as a yellow oil. LC-MS: MS (ES⁺): m/z=543.2 [M+H⁺].

### 58.3 Preparation of Compound 4

To a solution of tert-butyl-N-[2-[2-[2-[benzyl(methyl)amino]ethoxy]ethoxy]ethyl]-N-tert-butoxycarbonyl-carbamate (6.70 g, 14.8 mmol, 1.0 eq) in TFE (70 mL) was added Pd(OH)₂/C (700 mg, 10% purity). The suspension was stirred under H₂ (50 psi) at 30° C. for 16 h. The mixture was filtered. The filtrate was concentrated to give tert-butyl-N-tert-butoxycarbonyl-N-[2-[2-[2-(methylamino)ethoxy]ethoxy]ethyl]-carbamate (5.40 g, crude) as a light yellow oil. LC-MS: MS (ES⁺): m/z=363.1 [M+H⁺].
58.4 Preparation of Compound 5

To a solution of tert-butyl N-tert-butoxycarbonyl-N-[2-[2-[2-(methylamino)ethoxy]ethoxy]ethyl]carbamate (5.40 g, 14.9 mmol, 1.0 eq) and methyl (E)-4-bromobut-2-enoate (2.40 g, 13.4 mmol, 0.9 eq) in DCM (100 mL) was added DIEA (2.89 g, 22.4 mmol, 1.5 eq). The mixture was stirred at 25° C. for 16 h. The mixture was concentrated to give a residue. The residue was purified by prep-HPLC (column: Phenomenex luna C18 (250*70 mm, 15 um); mobile phase: [water(1OmM NH₄HCO₃)-ACN]; B%: 55%-85ACN %, 20 min) to give methyl-(E)-4-[2-[2-[2-[bis(tert-butoxycarbonyl)amino]ethoxy]ethoxy]ethyl-methyl-amino]but-2-enoate (4.70 g, 10.2 mmol, 68% yield, 100% purity) as a light yellow oil. LC-MS: MS (ES⁺): RT=0.992 min, m/z=461.1 [M+H⁺]. ¹H NMR (400 MHz, CDCl₃): δ7.03-6.90 (m, 1H), 6.05-5.93 (m, 1H), 3.82-3.76 (m, 2H), 3.74 (s, 3H), 3.63-3.56 (m, 8H), 3.26-3.15 (m, 2H), 2.60 (t, J=6.0 Hz, 2H), 2.29 (s, 3H), 1.50 (s, 18H).
58.5 Preparation of Compound 6

To a solution of methyl-(E)-4-[2-[2-[2-[bis(tert-butoxycarbonyl)amino]ethoxy]ethoxy]ethyl-methyl-amino]but-2-enoate (150 mg, 326 μmol, 1.0 equiv) in THF (2 mL) and H₂O (1 mL) was added KOH (24 mg, 0.42 mmol, 1.3 equiv). The mixture was stirred at 50° C. for 3 h. The mixture was concentrated to give crude (E)-4-[2-[2-[2-[bis(tert-butoxycarbonyl)amino]ethoxy]ethoxy]ethyl-methyl-amino]but-2-enoic acid (145 mg, crude) as a light yellow solid. LC-MS: MS (ES⁺): RT=0.658 min, m/z=447.3 [M+H⁺].
58.6 Preparation of Compound 8

To a solution of (E)-4-[2-[2-[2-[bis(tert-butoxycarbonyl)amino]ethoxy]ethoxy]ethyl-methyl-amino]but-2-enoic acid (145 mg, 325 μmol, 1.0 equiv) and 4-amino-N-[4-[2-(dimethylamino)-2-oxo-ethyl]-2,3-dimethyl-phenyl]-1-[(3R)-3-piperidyl]pyrazolo[3,4-d]pyrimidine-3-carboxamide (184 mg, 326 μmol, 1.0 equiv, TFA salt) in DMF (3.0 mL) were added DIEA (168 mg, 1.30 mmol, 4.0 equiv) and HATU (148 mg, 390 μmol, 1.2 equiv). The mixture was stirred at 25° C. for 1 h. The mixture was concentrated to give a residue. The residue was purified by prep-TLC (SiO₂, DCM: MeOH=10: 1) to give the desired product tert-butyl-N-[2-[2-[2-[[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-methyl-amino]ethoxy]ethoxy]ethyl]-N-tert-butoxycarbonyl-carbamate (130 mg, 148 μmol, 46% yield) as a colorless gum. LC-MS: MS (ES⁺): m/z=879.7 [M+H⁺].
58.7 Preparation of Compound 9

To a solution of tert-butyl-N-[2-[2-[2-[[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-methyl-amino]ethoxy]ethoxy]ethyl]-N-tert-butoxycarbonyl-carbamate (130 mg, 148 μmol, 1.0 equiv) in DCM (2 mL) was added TFA (1.54 g, 13.5 mmol, 1.0 mL, 91 equiv). The mixture was stirred at 25° C. for 1 h. The mixture was concentrated to give crude 4-amino-11-[(3R)-1-[(E)-4-[2-[2-(2-aminoethoxy)ethoxy]ethyl-methyl-amino]but-2-enoyl]-3-piperidyl]-N-[4-[2-(dimethylamino)-2-oxo-ethyl]-2,3-dimethyl-phenyl]-pyrazolo[3,4-d]pyrimidine-3-carboxamide (117 mg, crude, TFA salt) as a light yellow gum. LC-MS: MS (ES⁺): m/z=679.6 [M+H⁺].
58.8 Preparation of Compound I-58

To a solution of 4-amino-1-[(3R)-1-[(E)-4-[2-[2-(2-aminoethoxy)ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (117 mg, 148 μmol, 1.0 equiv, TFA salt) and 4-[[(7R)-8-cyclopentyl-7-ethyl-5-methyl-6-oxo-7H-pteridin-2-yl]amino]-3-methoxy-benzoic acid (62 mg, 0.15 mmol, 1.0 equiv) in DMF (1.5 mL) were added DIEA (76 mg, 0.59 mmol, 4.0 equiv) and HATU (67 mg, 0.18 mmol, 1.2 equiv). The mixture was stirred at 25° C. for 1 h. The mixture was purified by prep-HPLC (column: Phenomenex luna C18 150*25 mm* 10 um; mobile phase: [water(0.225% FA)-ACN]; B%: 9%-39%, 10 min) to give 4-amino-1-[(3R)-1-[(E)-4-[22[2-2-[[4-[[(7R)-8-cyclopentyl-7-ethyl-5-methyl-6-oxo-7H-pteridin-2-yl]amino]-3-methoxy-benzoyl]amino]ethoxy]ethoxy]ethyl-methyl-amino]but-2-enoyl]-3-piperidyl]-N—[4-[2-(dimethylamino)-2-oxo-ethyl]-2,3-dimethyl-phenyl]pyrazolo[3,4-d]pyrimidine-3-carboxamide (77 mg, 71 μmol, 48% yield, 100% purity) as a white solid. LC-MS: MS (ES′): RT=2.119 min, m/z=1086.6 [M+H⁺]; LC-MS Method 10. ¹H NMR (400 MHz, MeOD): δ8.48-8.42 (m, 1H), 8.29-8.16 (m, 1H), 7.80-7.66 (m, 1H), 7.52-7.29 (m, 3H), 7.06-6.92 (m, 1H), 6.81-6.47 (m, 2H), 4.96-4.88 (m, 1H), 4.84-4.74 (m, 1H), 4.60-3.85 (m, 7H), 3.84-3.74 (m, 2H), 3.72-3.50 (m, 11H), 3.49-3.32 (m, 3H), 3.10 (s, 3H), 2.99 (s, 3H), 2.89-2.67 (m, 2H), 2.53-2.09 (m, 13H), 2.01-1.61 (m, 11H), 0.91-0.75 (m, 3H).

### Example 59—Synthesis of Compound I-59

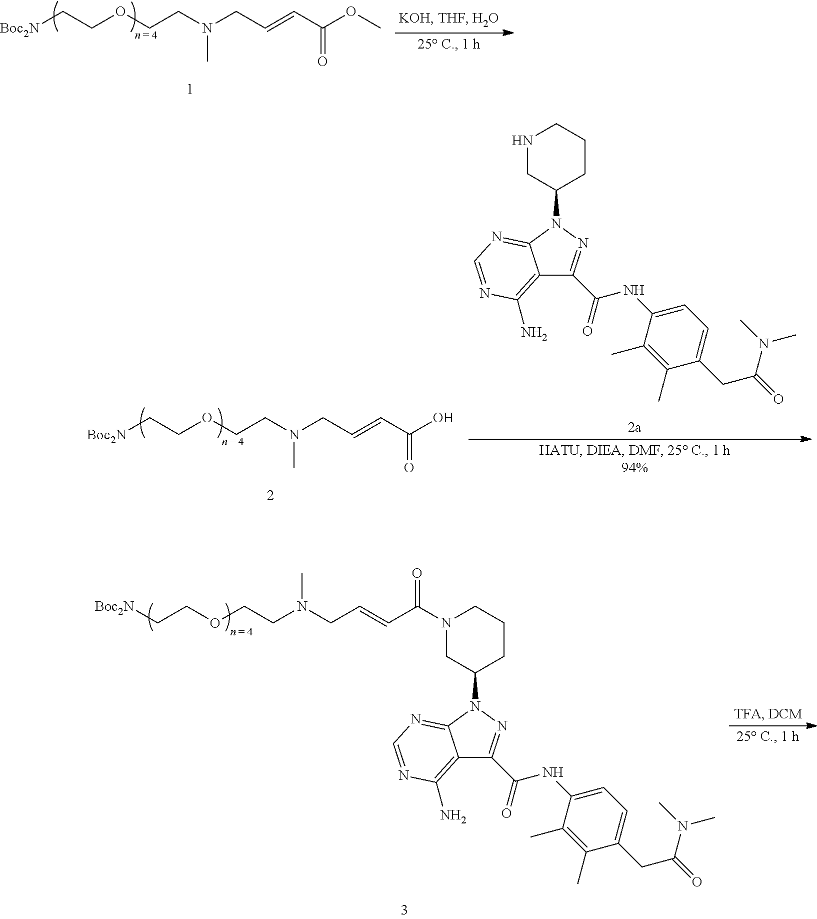

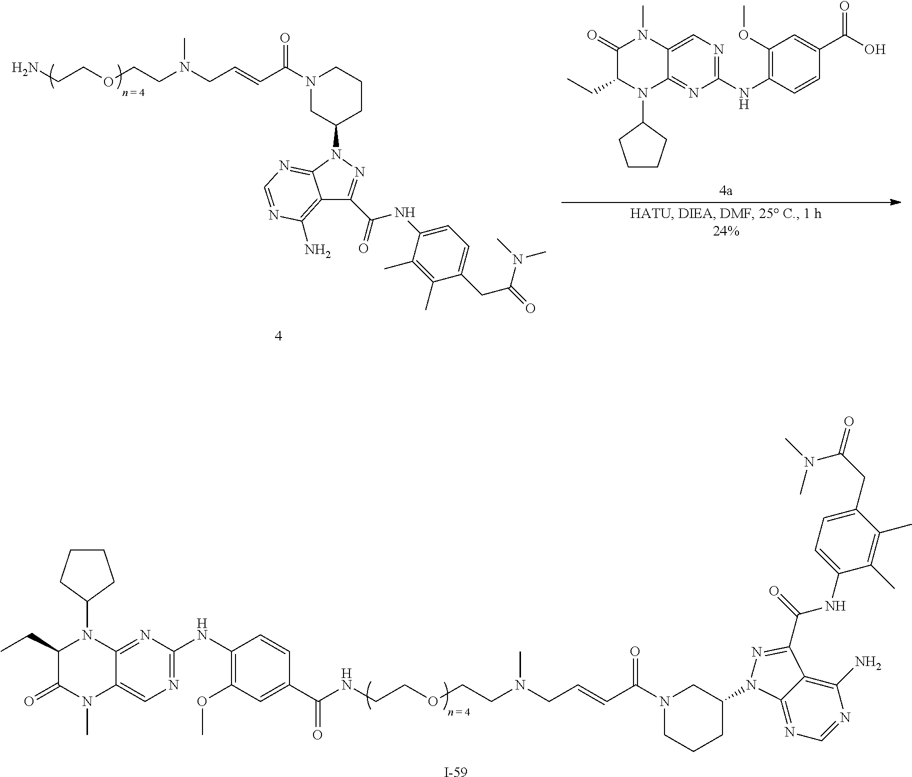

### 59.1 Preparation of Compound 2

To a solution of methyl-(E)-4-[2-[2-[2-[2-[2-[bis(tert-butoxycarbonyl)amino]ethoxy]ethoxy]ethoxy]ethoxy]-ethyl-methyl-amino]but-2-enoate (0.30 g, 0.55 mmol, 1.0 equiv) in THF (2 mL) and H₂O (1 mL) was added KOH (90 mg, 1.6 mmol, 3.0 equiv). The mixture was stirred at 25° C. for 1 h. The solution was concentrated to give (E)-4-[2-[2-[2-[2-[2-[bis(tert-butoxycarbonyl)amino]ethoxy]ethoxy]ethoxy]ethoxy]ethyl-methyl-amino]but-2-enoic acid (0.29 g, 0.54 mmol) as a yellow oil. LC-MS: MS (ES⁺): m/z=535.3 [M+H⁺].

### 59.2 Preparation of Compound 3

To a solution of 4-amino-N-[4-[2-(dimethylamino)-2-oxo-ethyl]-2,3-dimethyl-phenyl]-1-[(3R)-3-piperidyl]pyrazolo[3,4-d]pyrimidine-3-carboxamide (0.26 g, 0.57 mmol, 1.3 equiv, TFA salt) and (E)-4-[2-[2-[2-[2-[2-[bis(tert-butoxycarbonyl)amino]ethoxy]ethoxy]ethoxy]ethoxy]ethyl-methyl-amino]but-2-enoic acid (0.29 g, 0.54 mmol, 1.2 equiv) in DMF (4 mL) were added HATU (0.24 g, 0.63 mmol, 1.4 equiv) and DIEA (0.18 g, 1.4 mmol, 3.0 equiv). The mixture was stirred at 25° C. for 1 h. The solution was purified by prep-HPLC (column: Waters Xbridge C18 150*50 mm* 10 um; mobile phase: [water(1OmM NH₄HCO₃)-ACN]; B%: 40%-70%, 10 min) to give tert-butyl-N-[2-[2-[2-[2-[2-[[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-methyl-amino]ethoxy]ethoxy]ethoxy]ethoxy]ethyl]-N-tert-butoxycarbonyl-carbamate (0.41 g, 0.42 mmol, 94% yield) as a yellow oil. LC-MS: MS (ES⁺): RT=0.617 min, m/z=967.4 [M+H⁺].

### 59.3 Preparation of Compound 4

To a solution of tert-butyl-N-[2-[2-[2-[2-[2-[[(E)-4-[(3R)-3-[4-amino-3-[[4-[2-(dimethylamino)-2-oxo-ethyl]-2,3-dimethyl-phenyl]carbamoyl]pyrazolo[3,4-d]pyrimidin-1-yl]-1-piperidyl]-4-oxo-but-2-enyl]-methyl-amino]ethoxy]ethoxy]ethoxy]ethoxy]ethyl]-N-tert-butoxycarbonyl-carbamate (100 mg, 103 μmol, 1.0 equiv) in DCM (2 mL) was added TFA (770 mg, 6.75 mmol, 65 equiv). The mixture was stirred at 25° C. for 1 h. The solution was concentrated to give crude 4-amino-1-[(3R)-1-[(E)-4-[2-[2-[2-[2-(2-aminoethoxy)ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (90 mg, 0.10 mmol, TFA salt) as a yellow oil. LC-MS: MS (ES⁺): RT=0.643 min, m/z=767.2 [M+H⁺].

### 59.4 Preparation of Compound I-59

To a solution of 4-amino-1-[(3R)-1-[(E)-4-[2-[2-[2-[2-(2-aminoethoxy)ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (90 mg, 0.10 mmol, 1.0 equiv, TFA salt) and 4-[[(7R)-8-cyclopentyl-7-ethyl-5-methyl-6-oxo-7H-pteridin-2-yl]amino]-3-methoxy-benzoic acid (55 mg, 0.13 mmol, 1.3 equiv) in DMF (2 mL) were added HATU (58 mg, 0.15 mmol, 1.5 equiv) and DIEA (40 mg, 0.31 mmol, 3.0 equiv). The mixture was stirred at 25° C. for 1 h. The solution was purified by prep-HPLC (column: Phenomenex luna C18 150*25 mm* 10 um; mobile phase: [water(0.225% FA)-ACN]; B%: 11%-41%, 10 min) to give 4-amino-1-[(3R)-1-[(E)-4-[2-[2-1[2-[2-[2-1[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (30 mg, 25 μmol, 24% yield, 98% purity) as a yellow oil. ¹H NMR (400 MHz, METHANOL-d₄): δ8.44 (d, J=9.0 Hz, 1H), 8.21 (d, J=10.4 Hz, 1H), 7.71 (s, 1H), 7.48-7.33 (m, 3H), 6.98 (d, J=8.2 Hz, 1H), 6.84-6.52 (m, 2H), 4.80 (s, 1H), 4.56 (d, J=13.0 Hz, 1H), 4.43 (t, J=7.6 Hz, 1H), 4.24 (dd, J=3.4, 7.6 Hz, 1H), 4.19 (d, J=13.2 Hz, 1H), 4.05 (dd, J=15.0, 18.2 Hz, 1H), 3.95 (s, 3H), 3.93-3.81 (m, 1H), 3.76 (s, 2H), 3.69-3.45 (m, 20H), 3.43-3.33 (m, 2H), 3.28 (s, 3H), 3.10 (s, 3H), 2.98 (s, 3H), 2.86 (s, 1H), 2.74 (s, 1H), 2.54-2.31 (m, 4H), 2.24 (d, J=4.8 Hz, 4H), 2.19 (s, 3H), 2.16-1.93 (m, 3H), 1.93-1.72 (m, 6H), 1.71-1.63 (m, 3H), 0.82 (t, J=7.4 Hz, 3H). LC-MS: MS (ES⁺): RT=2.153 min, m/z=1174.6 [M+H⁺]; LC-MS Method 10.

### Example 60—Synthesis of Compound I-60

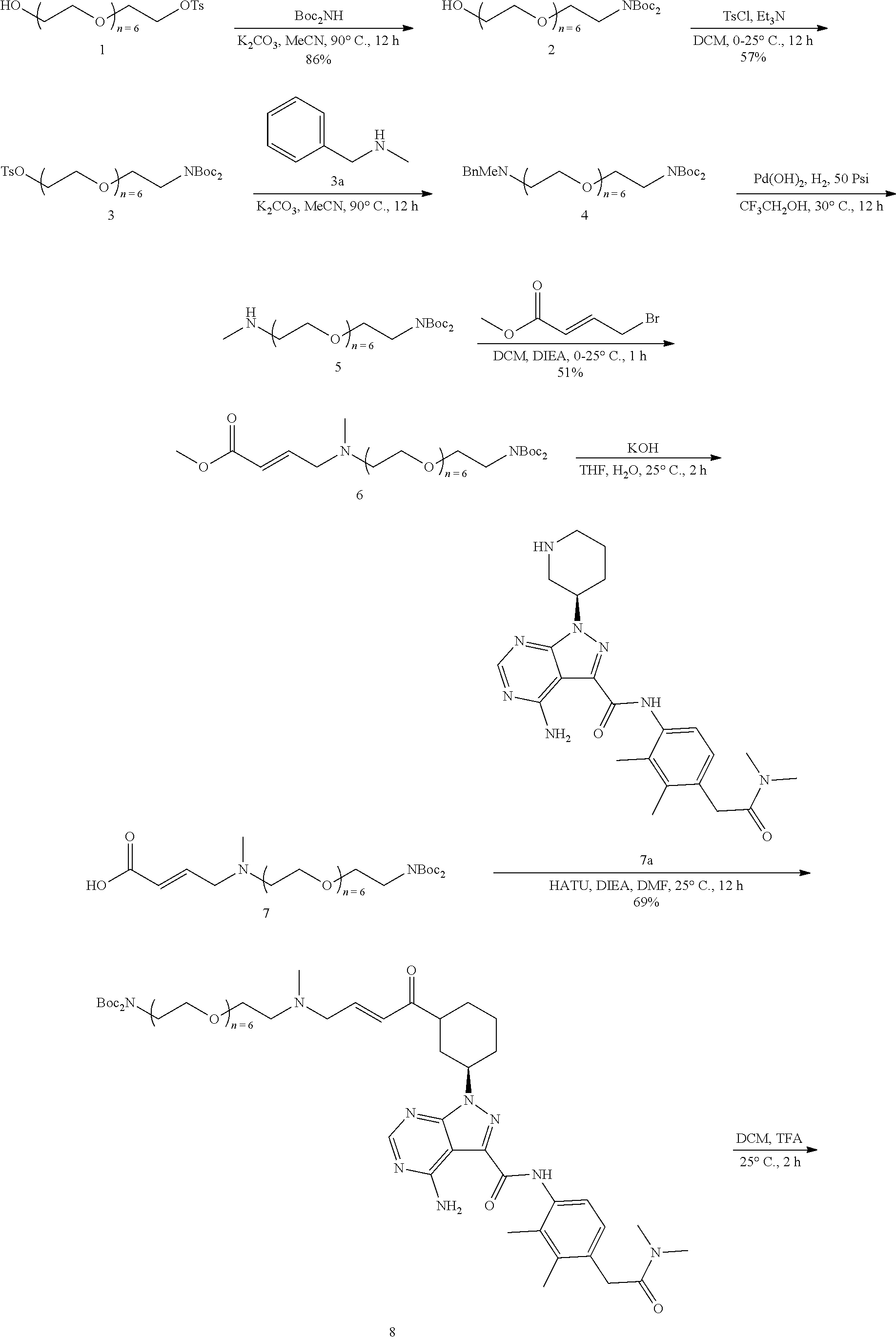

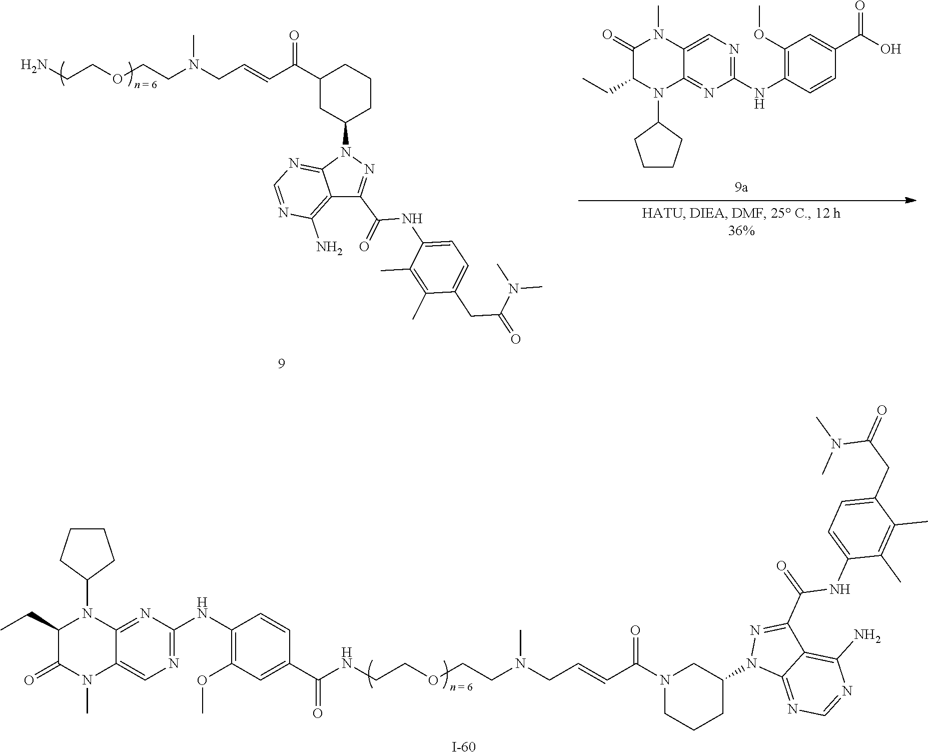

Synthetic route of compound 1 is described in the preparation of Compound I-12.

### 60.1 Preparation of Compound 2

To a solution of 2-[2-[2-[2-[2-[2-(2-hydroxyethoxy)ethoxy]ethoxy]ethoxy]ethoxy]ethoxy]ethyl 4-methylbenzenesulfonate (4.80 g, 9.99 mmol, 1.0 equiv) in MeCN (50 mL) was added K₂CO₃ (4.14 g, 30.0 mmol, 3.0 equiv) and tert-butyl N-tertbutoxycarbonylcarbamate (3.26 g, 15.0 mmol, 1.5 equiv). The mixture was stirred at 90° C. for 12 h. The mixture was filtered. The filtrate was concentrated to give a residue. The residue was purified by silica column chromatography on silica gel (Dichloromethane: Methanol from 100/1 to 10/1) to give the desired product tert-butyl-N-tert-butoxycarbonyl-N-[2-[2-[2-[2-[2-[2-(2-hydroxyethoxy) ethoxy]ethoxy]ethoxy]ethoxy]ethoxy]ethyl]carbamate (4.50 g, 8.56 mmol, 86% yield) as a colorless oil. LC-MS: MS (ES⁺): RT=0.853 min, m/z=543.3 [M+18⁺].

### 60.2 Preparation of Compound 3

To a solution of tert-butyl-N-tert-butoxycarbonyl-N-[2-[2-[2-[2-[2-[2-(2-hydroxyethoxy)ethoxy]ethoxy]ethoxy]ethoxy]ethoxy]ethyl]carbamate (4.50 g, 8.56 mmol, 1.0 equiv) in DCM (60 mL) were added TEA (1.73 g, 17.1 mmol, 2.0 equiv) and TosCl (2.45 g, 12.8 mmol, 1.5 equiv) at 0° C. The mixture was stirred at 25° C. for 12 h. The reaction mixture was washed with brine (3×50 mL), dried over by sodium sulfate and filtered. The filtrate was concentrated under reduced pressure to give a residue. The residue was purified by silica column chromatography on silica gel (Petroleum ether: Ethyl acetate from 10/1 to 0/1) to give the desired product 2-[21[2-[2-[2-[2-[bis(tert-butoxycarbonyl)amino]ethoxy]ethoxy]ethoxy]ethoxy]ethoxy]ethoxy]ethyl-4-methylbenzenesulfonate (3.30 g, 4.85 mmol, 57% yield) as a colorless oil. ¹H NMR (400 MHz, CDCl₃): δ7.80 (d, J=8.4 Hz, 2H), 7.35 (d, J=8.0 Hz, 2H), 4.19-4.14 (m, 2H), 3.82-3.75 (m, 2H), 3.71-3.68 (m, 2H), 3.66-3.60 (m, 18H), 3.59 (s, 4H), 2.45 (s, 3H), 1.50 (s, 17H).

### 60.3 Preparation of Compound 4

To a solution of 2-[2-[2-[2-[2-[2-[2-[bis(tert-butoxycarbonyl)amino]ethoxy]ethoxy]ethoxy]ethoxy]ethoxy]ethoxy]ethyl4-methylbenzenesulfonate (3.30 g, 4.85 mmol, 1.0 equiv) in MeCN (50 mL) were added K₂CO₃ (1.01 g, 7.28 mmol, 1.5 equiv) and Nmethyl-1-phenyl-methanamine (0.71 g, 5.83 mmol, 1.2 equiv). The mixture was stirred at 90° C. for 12 h. The mixture was filtered. The filtrate was concentrated to give a residue. The residue was purified by silica gel column chromatography (Dichloromethane: Methanol=100/1 to 10/1) to give the crude product tert-butyl-N-[2-[2-[2-[2-[2-[2-[2-[benzyl(methyl)amino]ethoxy]ethoxy]ethoxy]ethoxy]ethoxy]ethoxy]ethyl]-N-tertbutoxycarbonyl-carbamate (2.80 g, crude) as a yellow oil. LC-MS: MS (ES⁺): RT=0.625 min, m/z=629.3 [M+H⁺].

### 60.4 Preparation of Compound 5

To a solution of tert-butyl-N—[2-[2-[2-[2-[2-[2-[2-[benzyl(methyl)amino]ethoxy]ethoxy]ethoxy]ethoxy]ethoxy]ethoxy]ethyl]-Ntert-tbutoxycarbonyl-carbamate (2.80 g, 4.45 mmol, 1.0 equiv) in CF₃CH₂OH (30 mL) was added Pd(OH)₂/C (0.30 g, 10% purity) under N₂. The suspension was degassed under vacuum and purged with H₂ for several times. The mixture was stirred under H₂ (50 psi) at 30° C. for 12 h. The mixture was filtered. The filtrate was concentrated to give the crude product tert-butyl-N-tert-butoxycarbonyl-N-[2-[2-[2-[2-[2-[2-[2-(methylamino)ethoxy]ethoxy]ethoxy]ethoxy]ethoxy]ethoxy]ethyl]carbamate (2.60 g, crude) as a colorless oil.

### 60.5 Preparation of Compound 6

To a solution of tert-butyl-N-tert-butoxycarbonyl-N-[2-[2-[2-[2-[2-[2-[2-(methylamino)ethoxy]ethoxy]ethoxy]ethoxy]ethoxy]ethoxy]ethyl]carbamate (2.50 g, 4.64 mmol, 1.0 equiv) in DCM (30 mL) were added DIEA (1.20 g, 9.28 mmol, 1.6 mL, 2.0 equiv) and methyl €-4-bromobut-2-enoate (0.75 g, 4.18 mmol, 0.5 mL, 0.9 equiv) at 0° C. The mixture was stirred at 25° C. for 1 h. The mixture was filtered. The filtrate was concentrated to give a residue. The residue was purified by prep-HPLC (column: Waters Xbridge 150*25 mm* 5 um; mobile phase: [water(10 mM NH₄HCO₃)-ACN]; B%: 40%-70%, 9 min) to give the desired product methyl (E)-4-[2-[2-[2-[2-[2-[2-[2-[bis(tert-butoxycarbonyl)amino]ethoxy]ethoxy]ethoxy]ethoxy]ethoxy]ethoxy]ethyl-methylamino]but-2-enoate (1.50 g, 2.36 mmol, 51% yield) as a yellow oil. ¹H NMR (400 MHz, CDCl₃): δ7.04-6.90 (m, 1H), 6.01 (d, J=15.6 Hz, 1H), 3.81-3.77 (m, 2H), 3.74 (d, J=1.6 Hz, 3H), 3.67-3.63 (m, 16H), 3.62-3.59 (m, 8H), 3.35-3.21 (m, 2H), 2.66 (d, J=5.6 Hz, 2H), 2.32 (s, 3H), 1.50 (s, 18H). LC-MS: MS (ES⁺): RT=0.992 min, m/z=637.4 [M+H⁺].

### 60.6 Preparation of Compound 7

To a solution of methyl-(E)-4-[2-[2-[2-[2-[2-1[2-1[2-[bis(tertbutoxycarbonyl)amino]ethoxy]ethoxy]ethoxy]ethoxy]ethoxy]ethoxy]ethyl-methyl-amino]but-2-enoate (350 mg, 0.55 mmol, 1.0 equiv) in THF (3 mL) and H₂O (3 mL) was added KOH (46 mg, 0.8 mmol, 1.5 equiv). The mixture was stirred at 25° C. for 2 h. The mixture was concentrated to give the crude product (E)-4-[[21[2-[2-[2-[12-[2-[bis(tert-butoxycarbonyl)amino]ethoxy]ethoxy]ethoxy]ethoxy]ethoxy]ethoxy]ethyl-methylamino]but-2-enoic acid (350 mg, crude) as a yellow oil. LC-MS: MS (ES⁺): RT=0.762 min, m/z=623.3 [M+H⁺].

### 60.7 Preparation of Compound 8

To a solution of (E)-4-[2-[2-[2-[2-[2-[2-[2-[bis(tert-butoxycarbonyl)amino]ethoxy]ethoxy]ethoxy]ethoxy]ethoxy]ethoxy]ethylmethyl-amino]but-2-enoic acid (350 mg, 562 μmol, 1.0 equiv) in DMF (4 mL) was added 4-amino-N-[4-[2-(dimethylamino)-2-oxo-ethyl]-2,3-dimethyl-phenyl]-1-[(3R)-3-piperidyl]pyrazolo[3,4-d]pyrimidine-3-carboxamide (310 mg, 549 mol, 1.0 equiv, TFA salt), HATU (256 mg, 674 μmol, 1.2 equiv) and DIEA (145 mg, 1.12 mmol, 0.2 mL, 2.0 equiv). The mixture was stirred at 25° C. for 12 h. The mixture was filtered. The filtrate was concentrated to give a residue. The residue was purified by prep-HPLC (column: Waters Xbridge 150*25 mm* 5 um; mobile phase: [water(10 mM NH4HCO₃)-ACN]; B%: 41%-71%, 10 min) to give the desired product tert-butyl-N-[2-[2-[2-[2-[2-[2-[2-[[(E)-4-[(3R)-3-[4-amino-3-[[4-[2-(dimethylamino)-2-oxo-ethyl]-2,3-dimethylphenyl]carbamoyl]pyrazolo1[3,4-d]pyrimidin-1-yl]-1-piperidyl]-4-oxo-but-2-enyl]-methylamino]ethoxy]ethoxy]ethoxy]ethoxy]ethoxy]ethoxy]ethyl]-N-tert-butoxycarbonyl-carbamate (410 mg, 389 μmol, 69% yield) as a yellow oil. ¹H NMR (400 MHz, DMSO-d₆): δ 10.23-9.96 (m, 1H), 8.59 (s, 1H), 8.26 (s, 1H), 8.08 (s, 1H), 7.15 (s, 1H), 6.96 (d, J=8.0 Hz, 1H), 6.74-6.49 (m, 2H), 4.90-4.64 (m, 1H), 4.09 (s, 1H), 3.72 (s, 2H), 3.67-3.62 (m, 2H), 3.53-3.40 (m, 26H), 3.06 (s, 6H), 2.86 (s, 3H), 2.39 (s, 2H), 2.23-2.05 (m, 11H), 1.99 (s, 1H), 1.58 (s, 1H), 1.43 (s, 18H). LC-MS: MS (ES⁺): RT=1.008 min, m/z=1055.8 [M+H⁺].

### 60.8 Preparation of Compound 9

To a solution of tert-butyl-N-[2-[2-[2-[2-[2-[2-[2-[[(E)-4-[(3R)-3-[4-amino-3-[[4-[2-(dimethylamino)-2-oxo-ethyl]-2,3-dimethylphenyl]carbamoyl]pyrazolo[3,4-d]pyrimidin-1-yl]-1-piperidyl]-4-oxo-but-2-enyl]-methylamino]ethoxy]ethoxy]ethoxy]ethoxy]ethoxy]ethoxy]ethyl]-N-tert-butoxycarbonyl-carbamate (155 mg, 147 μmol, 1.0 equiv) in DCM (1.5 mL) was added TFA (494 mg, 4.33 mmol, 0.3 mL, 29.5 equiv). The mixture was stirred at 25° C. for 2 h. The mixture was concentrated to give crude product 4-amino-[(3R)-1-[(E)-4-[2-2-[2-[2-[2-[2-(2-aminoethoxy)ethoxy]ethoxy]ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (150 mg, crude, TFA salt) as a brown oil. LC-MS: MS (ES⁺): RT=0.733 min, m/z=856.5 [M+H⁺].

### 609 Preparation of Compound I-60

To a solution of 4-amino-1-[(3R)-1-[(E)-4-[2-[2-[2-[2-[2-[2-(2-aminoethoxy) ethoxy]ethoxy]ethoxy]ethoxy]ethoxy]ethyl-methylamino]but-2-enoyl]-3-piperidyl]-N-[4-[2-(dimethylamino)-2-oxo-ethyl]-2,3-dimethyl-phenyl]pyrazolo [3,4-d]pyrimidine-3-carboxamide (75 mg, 77 μmol, 1.0 equiv, TFA salt) in DMF (1 mL) were added 4-[(8-cyclopentyl-7-ethyl-5-methyl-6-oxo-7H-pteridin-2-yl)amino]-3-methoxy-benzoic acid (33 mg, 77 μmol, 1.0 equiv), HATU (35.3 mg, 92.9 μmol, 1.2 equiv) and DIEA (20.0 mg, 155 μmol, 2.0 equiv). The mixture was stirred at 25° C. for 12 h. The mixture was filtered. The filtrate was concentrated to give a residue. The residue was purified by prep-HPLC (column: Unisil 3-100 C₁₈ Ultra 150*50 mm*3 um; mobile phase: [water (0.225% FA)-ACN]; B%: 18%-48%, 10 min) to give the desired product 4-amino-1-[(3R)-1-[(E)-4-[2-[2-[2-[2-[2-[2-[2-[[4-[(8-cyclopentyl-7-ethyl-5-methyl-6-oxo-7H-pteridin-2-yl)amino]-3-methoxybenzoyl]amino]ethoxy]ethoxy]ethoxy]ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (35 mg, 28 μmol, 36% yield, 100% purity) as a off-white solid. ¹H NMR (400 MHz, DMSO-d₆): δ10.15 (d, J=11.2 Hz, 1H), 9.58 (s, 1H), 8.62 (s, 1H), 8.46 (s, 1H), 8.27 (d, J=4.6 Hz, 1H), 8.19 (s, 1H), 8.13 (s, 1H), 7.79 (s, 1H), 7.54-7.49 (m, 2H), 7.14 (d, J=4.4 Hz, 1H), 7.02-6.84 (m, 2H), 6.71-6.54 (m, 1H), 4.87-4.69 (m, 1H), 4.38-4.21 (m, 3H), 3.92 (s, 6H), 3.77-3.68 (m, 4H), 3.58-3.45 (m, 24H), 3.44-3.40 (m, 3H), 3.23 (s, 3H), 3.06 (s, 3H), 2.86 (s, 3H), 2.75 (d, J=20.0 Hz, 3H), 2.13 (d, J=13.6 Hz, 8H), 1.98 (s, 2H), 1.85 (dd, J=7.6, 20.0 Hz, 4H), 1.76-1.46 (m, 7H), 0.75 (t, J=7.4 Hz, 3H). LC-MS: MS (ES⁺): RT=2.203 min, m/z=1263.1 [M+H⁺]; LC-MS Method 10.

### Example 61—Synthesis of Compound I-61

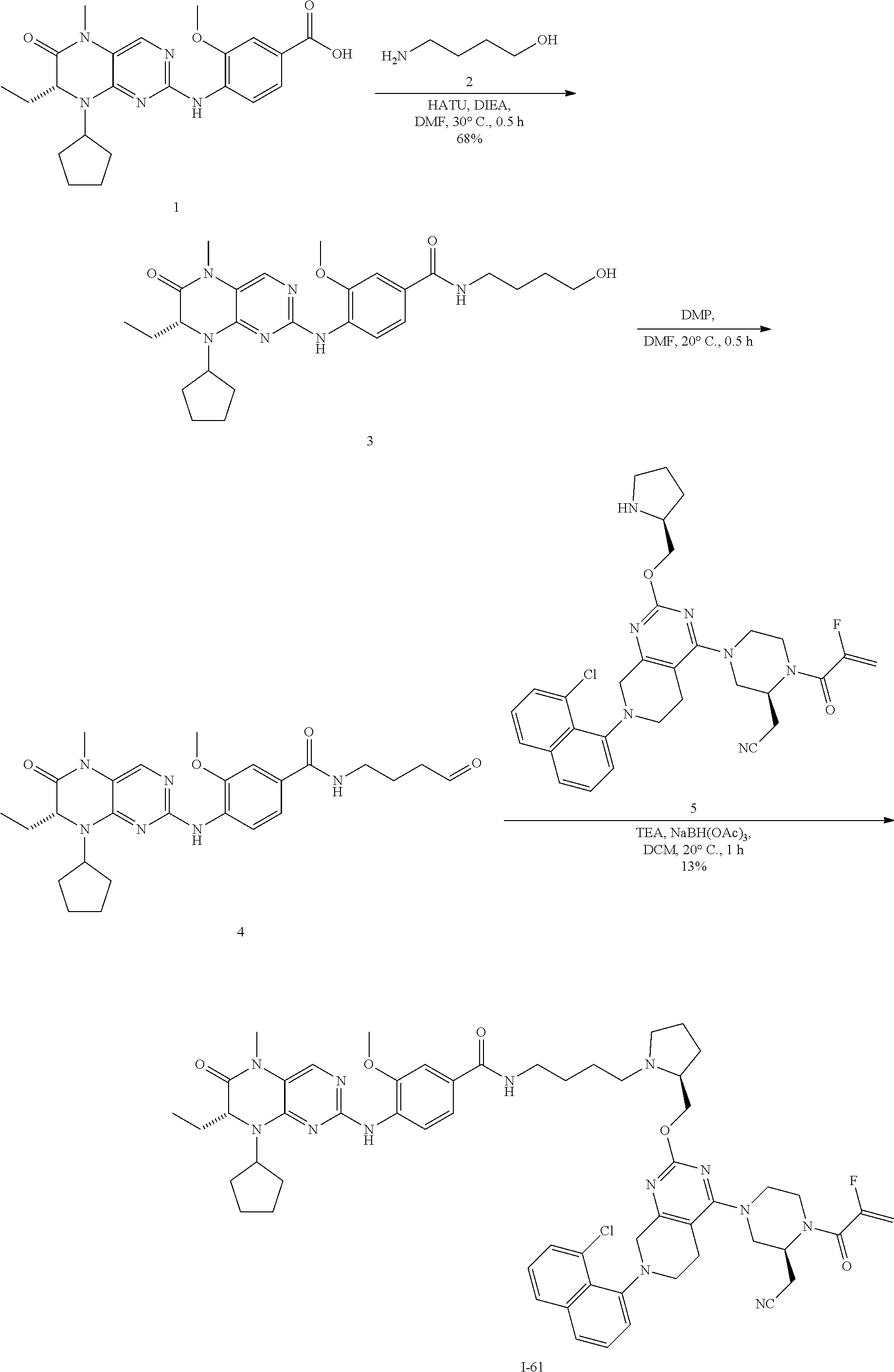

61.1 The preparation of compound 3

To a solution of compound 1 (100 mg, 235 μmol, 1.0 equiv) and compound 2 (26 mg, 291 μmol, 27 μL, 1.2 equiv) in DMF (1 mL) was added HATU (134 mg, 352 μmol, 1.5 equiv) and DIEA (89 mg, 688 μmol, 120 μL, 2.9 equiv). The mixture was stirred at 30° C. for 0.5 h. The reaction mixture was filtered and concentrated under reduced pressure to give a residue. The residue was purified by prep-HPLC (column: Waters Xbridge 150*25 mm* 5 um; mobile phase: [water (10 mM NH₄HCO₃)-ACN]; B%: 30%-60%, 9 min). Compound 3 (80 mg, 161 μmol, 68% yield) was obtained as a white solid. LC-MS: MS (ES⁺): RT=0.775 min, m/z=497.2 [M+1]+.

61.2 The preparation of compound 4

To a solution of compound 3 (40 mg, 80 μmol, 1.0 equiv) in DMF (1 mL) was added DMP (85 mg, 200 μmol, 62 μL, 2.4 equiv). The mixture was stirred at 20° C. for 0.5 h. The crude product 4 (40 mg, crude) was used into the next step without further purification. LC-MS: MS (ES⁺): RT=0.792 min, m/z=495.1 [M+1].

61.3 The preparation of Compound I-61

To a solution of compound 5 (40 mg, 67 μmol, 1.0 equiv, TFA salt) in DCM (1 mL) was added TEA (68 mg, 677 μmol, 94 μL, 10.0 equiv) and NaBH(OAc)₃ (143 mg, 677 μmol, 10.0 equiv). Then 4 (40 mg, crude) was added. The mixture was stirred at 20° C. for 0.5 h. The reaction mixture was partitioned between brine (20 mL) and DCM (20 mL). The organic phase was separated, dried over Na₂SO₄, filtered and concentrated under reduced pressure to give a residue. The residue was purified by column chromatography by prep-TLC (SiO₂, DCM: MeOH=9:1) and prep-HPLC (column: Phenomenex Gemini-NX C18 75*30 mm*3 um; mobile phase: [water(0.225% FA)-ACN]; B %: 30%-40%, 7 min) to give the desired compound N-(4-((S)-2-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidin-1-yl)butyl)-4-(((R)-8-cyclopentyl-7-ethyl-5-methyl-6-oxo-5,6,7,8-tetrahydropteridin-2-yl)amino)-3-methoxybenzamide (10 mg, 9 μmol, 13% yield, 97% purity, FA salt) as an off-white solid. ¹HNMR (400 MHz, CD₃OD): δ8.53 (s, 1H), 8.45 (d, J=9.3 Hz, 1H), 7.82-7.76 (m, 2H), 7.64 (d, J=8.4 Hz, 1H), 7.51-7.38 (m, 4H), 7.37-7.30 (m, 1H), 7.24 (t, J=8.5 Hz, 1H), 5.42-5.22 (m, 2H), 4.54-4.36 (m, 3H), 4.35-3.99 (m, 5H), 3.98-3.89 (m, 3H), 3.70-3.33 (m, 8H), 3.29-2.85 (m, 10H), 2.83-2.46 (m, 3H), 2.26-2.06 (m, 2H), 1.99-1.65 (m, 16H), 0.84 (t, J=7.4 Hz, 3H). LC-MS: MS (ES⁺): RT=2.146 min, m/z=535.0 [M/2+H⁺]; LC-MS Method 25.

### Example 62—Synthesis of Compound I-62

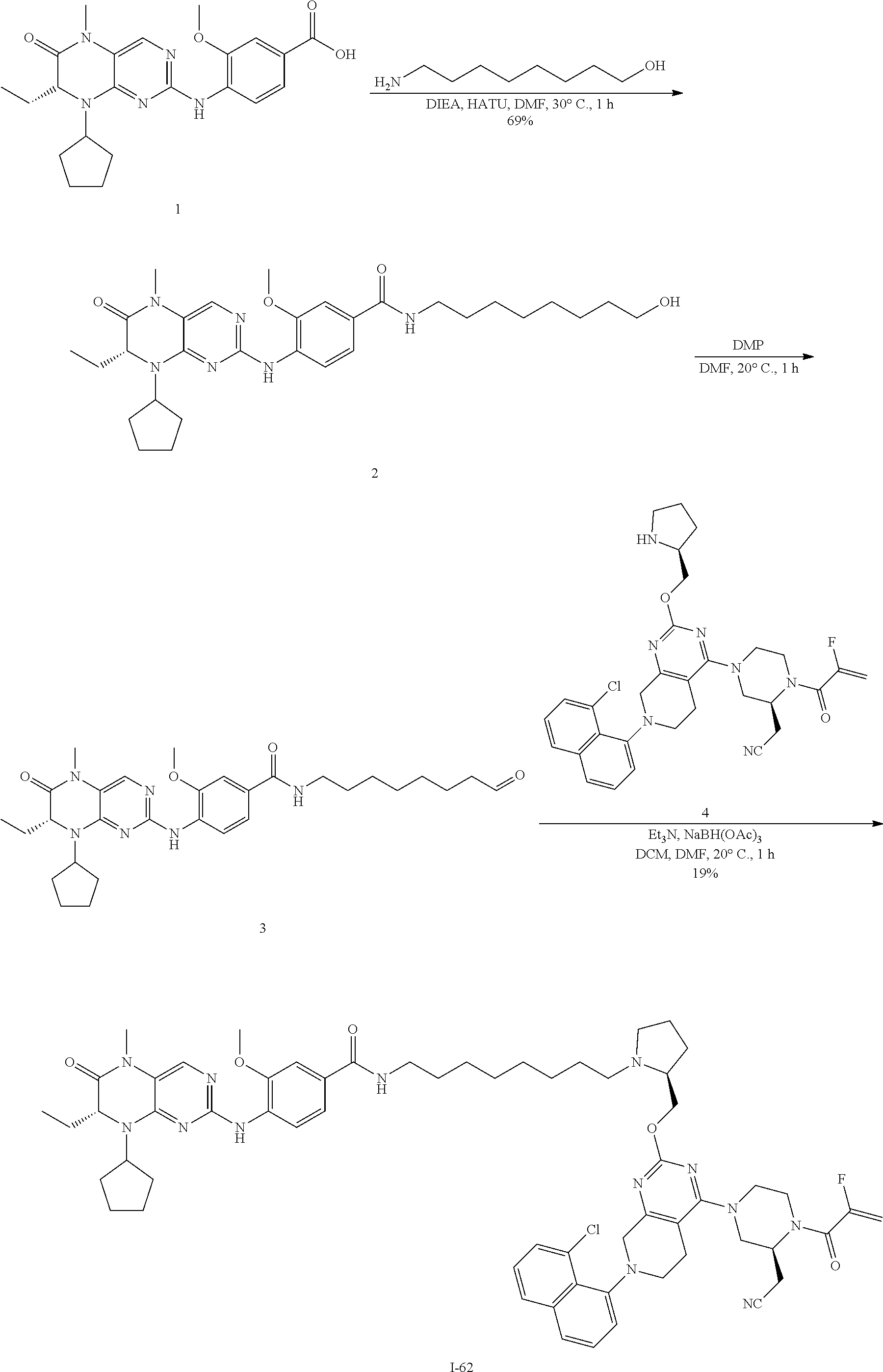

### 62.1 Preparation of Compound 2

To a solution of 4-[(8-cyclopentyl-7-ethyl-5-methyl-6-oxo-7H-pteridin-2-yl)amino]-3-methoxy-benzoic acid (100 mg, 235 μmol, 1.0 equiv), 8-aminooctan-1-ol (41 mg, 0.28 mmol, 1.2 equiv) and DIEA (152 mg, 1.18 mmol, 205 μL, 5.0 equiv) in DMF (2 mL) was added HATU (107 mg, 282 μmol, 1.2 equiv), and it was stirred at 30° C. for 1 h. H₂O (0.1 mL) was added to quench this reaction. The residue was purified by prep-HPLC (column: Waters Xbridge 150*25 mm*5 um; mobile phase: [water(10 mM NH₄HCO₃)-ACN]; B%: 42%-72%, 9 min) to afford 4-[[(7R)-8-cyclopentyl-7-ethyl-5-methyl-6-oxo-7H-pteridin-2-yl]amino]-N-(8-hydroxyoctyl)-3-methoxy-benzamide (89 mg, 0.16 mmol, 69% yield) as a white solid. LC-MS: MS (ES⁺): RT=0.831 min, m/z=553.3 [M+H⁺].

### 62.2 Preparation of Compound 3

To a solution of 4-[[(7R)-8-cyclopentyl-7-ethyl-5-methyl-6-oxo-7H-pteridin-2-yl]amino]-N-(8-hydroxyoctyl)-3-methoxy-benzamide (67 mg, 0.12 mmol,, 1.0 equiv) in DMF (1 mL) was added DMP (77 mg, 0.18 mmol, 56 μL, 1.5 equiv), and it was stirred at 20° C. for 1 h. The crude product 4-[[(7R)-8-cyclopentyl-7-ethyl-5-methyl-6-oxo-7H-pteridin-2-yl]amino]-3-methoxy-N-(8-oxooctyl)benzamide (60 mg, crude) was given in solution as a yellow liquid. LC-MS: MS (ES⁺): RT=0.775 min, m/z=551.2 [M+H⁺].

### 62.3 Preparation of Compound I-62

To a solution of 4-[[(7R)-8-cyclopentyl-7-ethyl-5-methyl-6-oxo-7H-pteridin-2-yl]amino]-3-methoxy-N-(8-oxooctyl)benzamide (60 mg, 0.11 mmol, 1.2 equiv) and 2-[(2S)-4-[7-(8-chloro-1-naphthyl)-2-[ [(2S)-pyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (64 mg, 91 μmol, 1.0 equiv, TFA salt) in DCM (2 mL) and DMF (1 mL) was added Et₃N (92 mg, 0.91 mmol, 0.1 mL, 10.0 equiv) and NaBH(OAc)₃ (192 mg, 910 μmol, 10.0 equiv), and then it was stirred at 20° C. for 1 h. To the reaction mixture was added water (50 mL) and the mixture was extracted with EtOAc (50 mL). The organic phase was washed with brine (3×50 mL), dried over anhydrous Na₂SO₄, filtered and concentrated in vacuum. The residue was purified by preparative TLC on silica gel (DCM: MeOH=8:1) to give a crude product, and then it was purified by prep-HPLC (column: Phenomenex Gemini-NX C18 75*30 mm*3 um; mobile phase: [water(0.225% FA)-ACN]; B%: 25%-55%, 7 min) to afford N-[8-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]octyl]-4-[[(7R)-8-cyclopentyl-7-ethyl-5-methyl-6-oxo-7H-pteridin-2-yl]amino]-3-methoxy-benzamide (21 mg, 18 μmol, 19% yield, 98% purity, FA salt) as a white solid. LC-MS: MS (ES′): RT=2.150 min, m/z=1124.4 [M+H⁺]. ¹H NMR (400 MHz, CD₃OD) δ8.46 (d, J=8.4 Hz, 1H), 7.87-7.75 (m, 2H), 7.65 (d, J=8 Hz, 1H), 7.54-7.41 (m, 4H), 7.37-7.25 (m, 2H), 5.44-5.23 (m, 2H), 4.55-4.40 (m, 3H), 4.37-3.94 (m, 8H), 3.74-3.34 (m, 9H), 3.28-3.08 (m, 5H), 3.07-2.57 (m, 5H), 2.23-2.10 (m, 2H), 2.01-1.54 (m, 16H), 1.45-1.25 (m, 8H), 0.84 (t, J=7.6 Hz, 3H).

### Example 63—Synthesis of Compound I-63

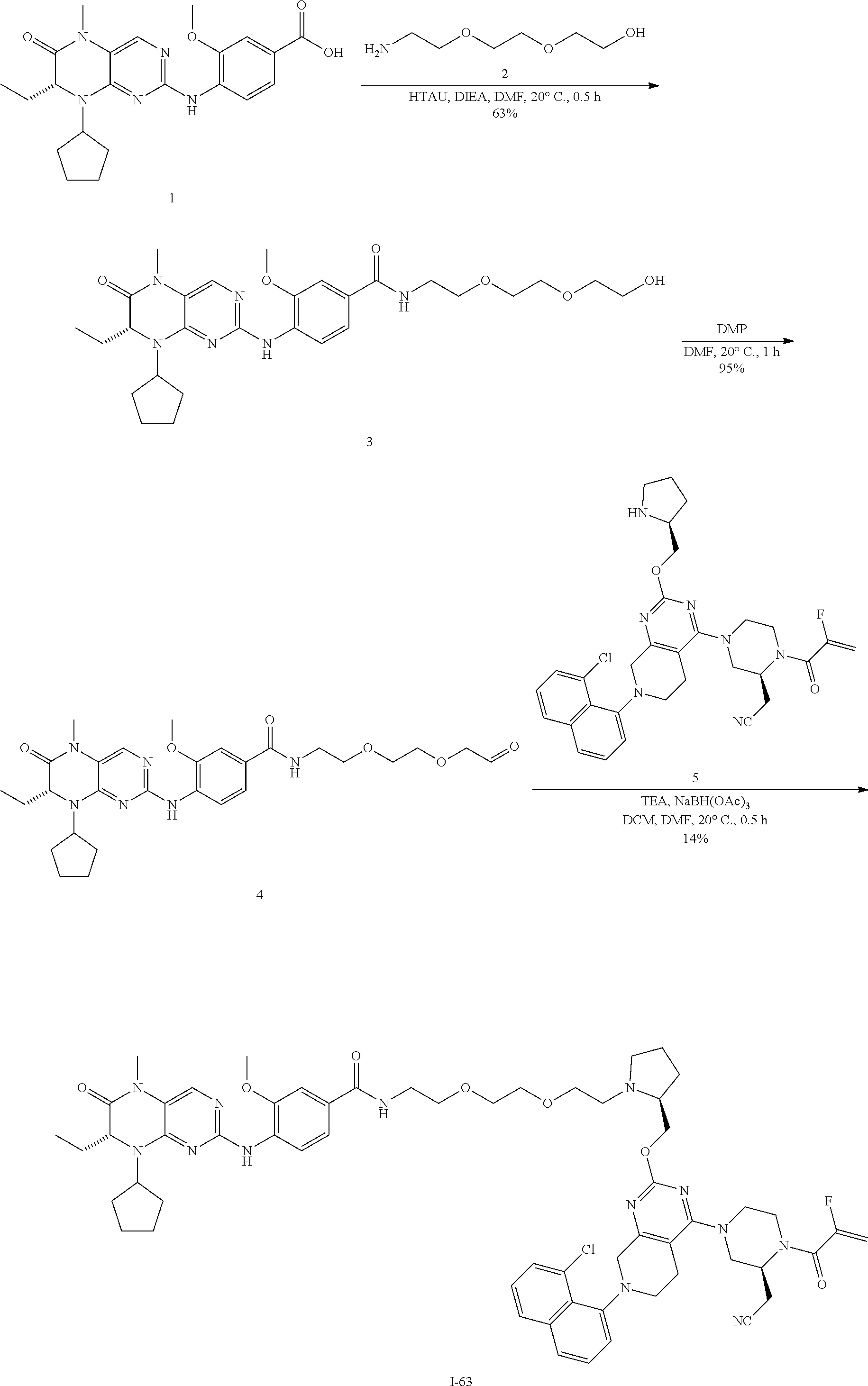

63.1 The preparation of compound 3

To a solution of compound 1 (110 mg, 259 μmol, 1.0 equiv) and compound 2 (38.6 mg, 259 μmol, 1.0 equiv) in DMF (1 mL) was added HATU (147 mg, 388 μmol, 1.5 equiv) and DIEA (100 mg, 776 μmol, 135 μL, 3.0 equiv). The mixture was stirred at 20° C. for 0.5 h. The reaction mixture was concentrated under reduced pressure to give a residue. The residue was purified by prep-HPLC (column: 3_Phenomenex Luna C18 75*30 mm*3 um; mobile phase: [water (0.1%TFA)-ACN]; B%: 20%-50%, 7 min). Compound 3 (90.0 mg, 162 μmol, 63% yield) was obtained as a white solid. LC-MS: MS (ES⁺): RT=0.781 min, m/z=557.3 [M+H⁺].

63.2 The preparation of compound 4

To a solution of compound 3 (90.0 mg, 162 μmol, 1.0 equiv) in DMF (1 mL) was added DMP (137 mg, 323 μmol, 100 μL, 2.0 equiv). The mixture was stirred at 20° C. for 1 h. The reaction mixture was used for next step without purification. LC-MS: MS (ES⁺): RT=0.789 min, m/z=555.4 [M+H⁺].

63.3 The preparation of Compound I-63

To a solution of compound 5 (70 mg, 99 μmol, 1.0 equiv, TFA) in DCM (4 mL) was added TEA (100 mg, 994 μmol, 138 μL, 10.0 equiv) and NaBH(OAc)₃ (210 mg, 994 μmol, 10.0 equiv). Then a solution of compound 4 (82 mg, 149 μmol, 1.5 equiv) in DMF (1.5 mL) was added drop-wise. The mixture was stirred at 20° C. for 0.5 h. The mixture was purified by prep-HPLC (column: Phenomenex Gemini-NX C18 75*30 mm*3 um; mobile phase: [water (0.225% FA)-ACN]; B %: 30%-40%, 7 min) to give the desired product N-(2-(2-(2-((S)-2-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidin-1-yl)ethoxy)ethoxy)ethyl)-4-(((R)-8-cyclopentyl-7-ethyl-5-methyl-6-oxo-5,6,7,8-tetrahydropteridin-2-yl)amino)-3-methoxybenzamide (16 mg, 13 μmol, 14% yield, 95% purity) as a yellow solid. ¹HNMR (400 MHz, CD₃OD): 6 8.56-8.50 (m, 1H), 8.49-8.44 (m, 1H), 7.83-7.75 (m, 2H), 7.65 (d, J=8.1 Hz, 1H), 7.52-7.41 (m, 4H), 7.38-7.31 (m, 1H), 7.28-7.20 (m, 1H), 5.42-5.21 (m, 2H), 4.53-4.21 (m, 6H), 4.18-3.99 (m, 2H), 3.96 (s, 3H), 3.78-3.58 (m, 1OH), 3.58-3.44 (m, 4H), 3.37 (m, 4H), 3.30-2.80 (m, 9H), 2.80-2.51 (m, 2H), 2.19-2.05 (m, 2H), 2.01-1.62 (m, 12H), 0.89-0.77 (m, 3H). LC-MS: MS (ES⁺): RT=2.149 min, m/z=1129.4 [M+H⁺]; LC-MS Method 25.

### Example 64—Synthesis of Compound I-64

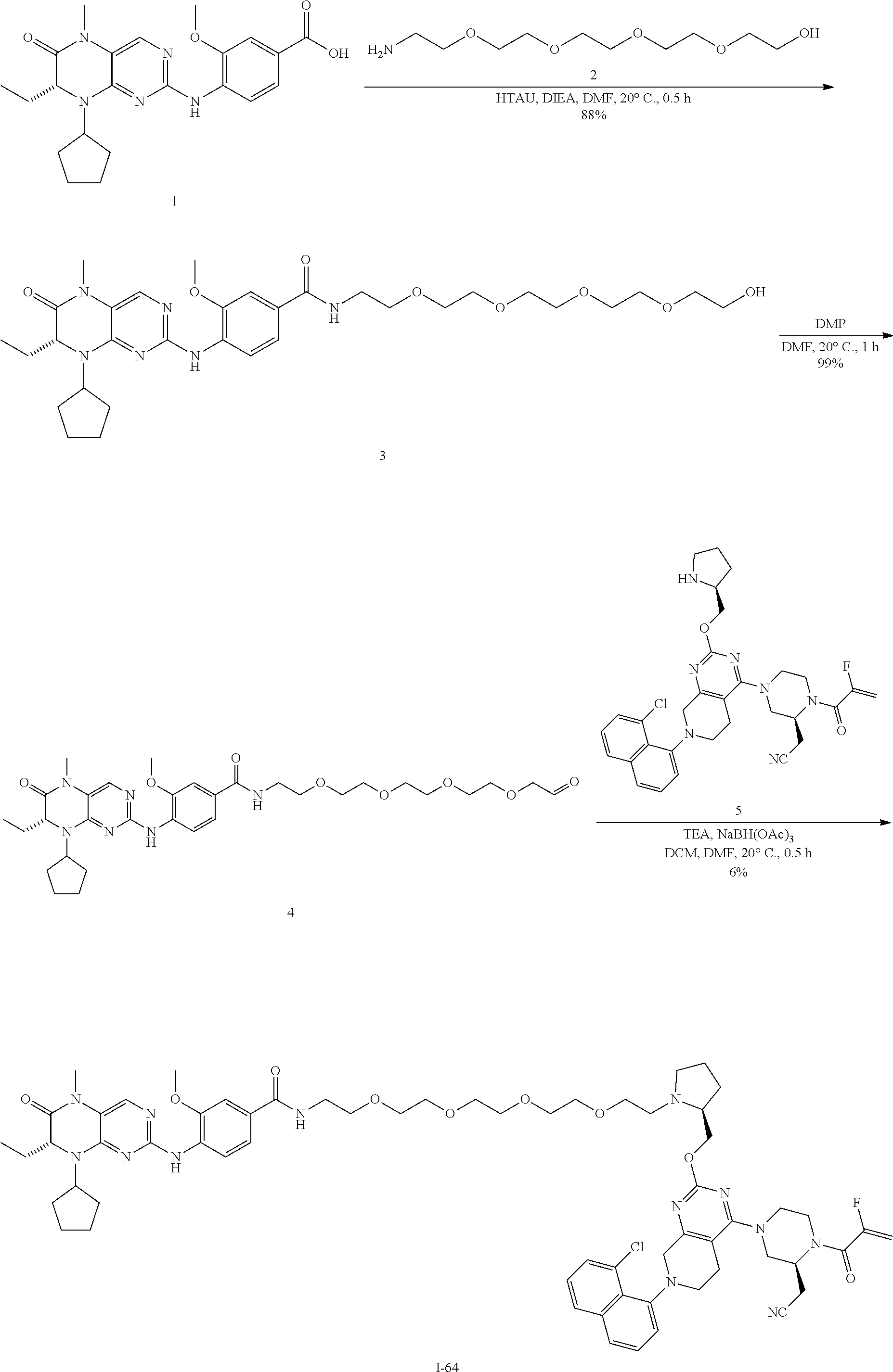

64.1 The preparation of compound 3

To a solution of compound 1 (120 mg, 282 μmol, 1.0 equiv) and compound 2 (67 mg, 282 μmol, 1.0 equiv) in DMF (2 mL) was added HATU (161 mg, 423 μmol, 1.5 equiv) and DIEA (109 mg, 846 μmol, 147 μL, 3.0 equiv). The mixture was stirred at 20° C. for 0.5 h. The reaction mixture was concentrated under reduced pressure to give a residue. The residue was purified by prep-HPLC (column: 3_Phenomenex Luna C18 75*30 mm*3 um; mobile phase: [water (0.1%TFA)-ACN]; B%: 19%-49%, 7 min). Compound 3 (160 mg, 248 μmol, 88% yield) was obtained as a colorless oil. LC-MS: MS (ES′): RT=0.762 min, m/z=645.4 [M+H⁺].

### 64.2 the Preparation of Compound 4

To a solution of compound 3 (80 mg, 124 μmol, 1 equiv) in DMF (1 mL) was added DMP (105 mg, 248 μmol, 77 μL, 2.0 equiv). The mixture was stirred at 20° C. for 1 h. The reaction mixture was used for next step without purification. LC-MS: MS (ES⁺): RT=0.789 min, m/z=643.4 [M+H⁺].

### 64.3 the Preparation of Compound I-64

To a solution of compound 5 (66 mg, 94 μmol, 1.0 equiv, TFA) in DCM (4 mL) was added TEA (95 mg, 937 μmol, 130 μL, 10.0 equiv) and NaBH(OAc)₃ (199 mg, 937 μmol, 10.0 equiv). Then a solution of compound 4 (78 mg, 122 μmol, 1.3 equiv) in DMF (1.5 mL) was added dropwise. The mixture was stirred at 20° C. for 1 h. The mixture was purified by prep-HPLC (column: Unisil 3-100 C18 Ultra 150*50 mm*3 um; mobile phase: [water(0.225% FA)-ACN]; B %: 20%-50%, 10 min) to give the desired product N-(14-((S)-2-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidin-1-yl)-3,6,9,12-tetraoxatetradecyl)-4-(((R)-8-cyclopentyl-7-ethyl-5-methyl-6-oxo-5,6,7,8-tetrahydropteridin-2-yl)amino)-3-methoxybenzamide (7.56 mg, 5.56 μmol, 6% yield, 93% purity, FA) as a yellow solid. ¹HNMR (400 MHz, CD₃OD): 6 8.48 (d, J=8.2 Hz, 1H), 7.82-7.75 (m, 2H), 7.67-7.63 (m, 1H), 7.53-7.42 (m, 4H), 7.38-7.32 (m, 1H), 7.30-7.23 (m, 1H), 5.41-5.22 (m, 2H), 4.60-4.33 (m, 3H), 4.32-4.19 (m, 4H), 4.18-4.07 (m, 2H), 4.06-3.94 (m, 4H), 3.73-3.46 (m, 23H), 3.25-2.86 (m, 9H), 2.77-2.47 (m, 3H), 2.04-1.64 (m, 14H), 0.87-0.81 (m, 3H). LC-MS: MS (ES⁺): RT=2.190 min, m/z=609.0 [M/2+H⁺]; LC-MS Method 25.

### Example 65—Synthesis of Compound I-65

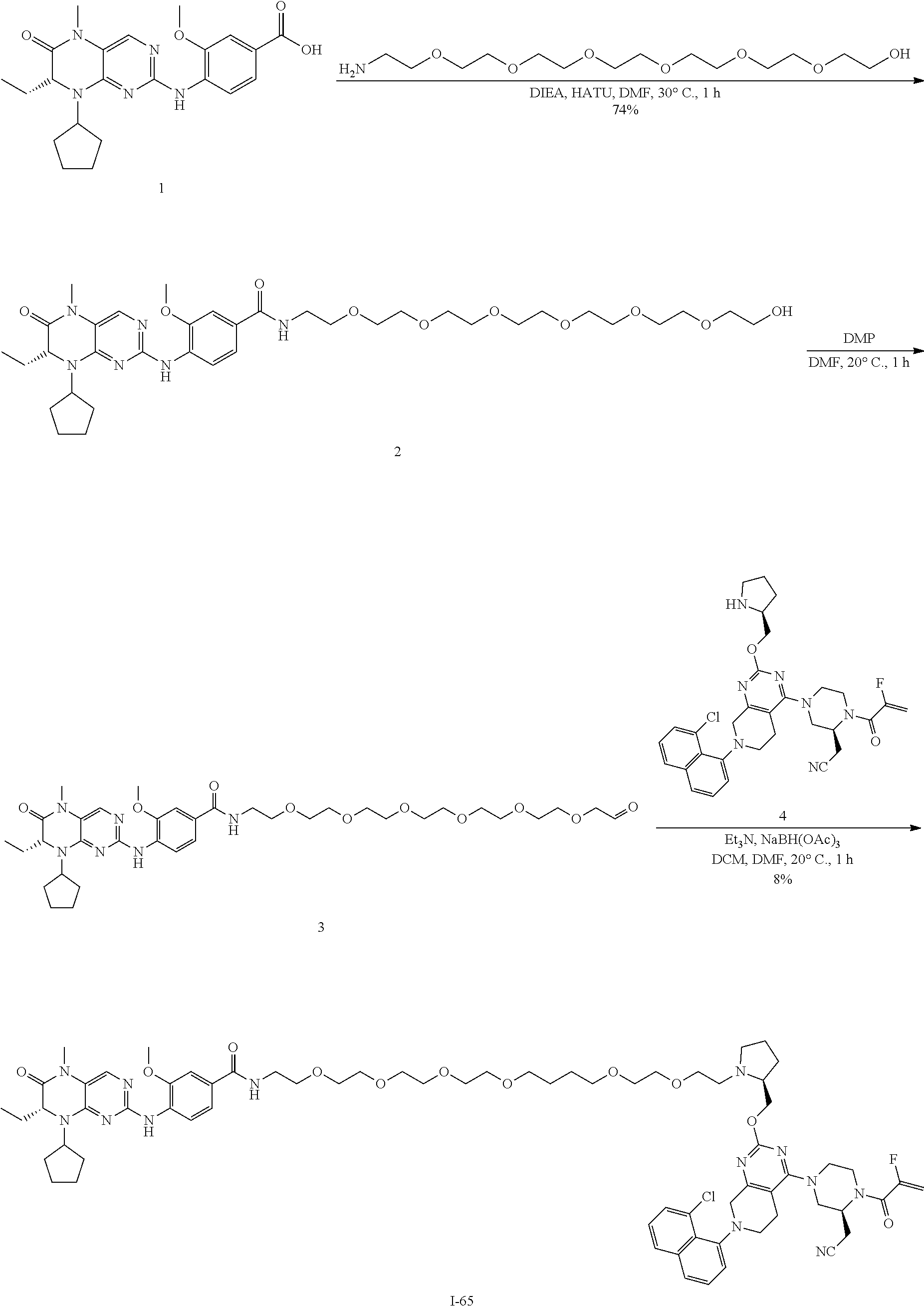

### 65.1 Preparation of Compound 2

To a solution of 4-[(8-cyclopentyl-7-ethyl-5-methyl-6-oxo-7H-pteridin-2-yl)amino]-3-methoxy-benzoic acid (100 mg, 235 μmol, 1.0 equiv), 2-[2-[2-[2-[2-[2-(2-aminoethoxy)ethoxy]ethoxy]ethoxy]ethoxy]ethoxy]ethanol (92 mg, 0.28 mmol, 1.2 equiv) and DIEA (152 mg, 1.18 mmol, 205 μL, 5.0 equiv) in DMF (2 mL) was added HATU (107 mg, 282 mol, 1.2 equiv), and it was stirred at 30° C. for 1 h. H₂O (0.1 mL) was added to quench this reaction. The residue was purified by prep-HPLC (column: Waters Xbridge 150*25 mm*5 um; mobile phase: [water(1OmM NH₄HCO₃)-ACN]; B%: 32%-62%, 9 min) to afford 4-[(8-cyclopentyl-7-ethyl-5-methyl-6-oxo-7H-pteridin-2-yl)amino]-N-[2-[2-[2-[2-[2-[2-(2-hydroxyethoxy)ethoxy]ethoxy]ethoxy]ethoxy]ethoxy]ethyl]-3-methoxy-benzamide (127 mg, 173 μmol, 74% yield) as a colorless oil. LC-MS: MS (ES⁺): RT=0.525 min, m/z=733.2 [M+H⁺].

### 65.2 Preparation of Compound 3

To a solution of 4-[(8-cyclopentyl-7-ethyl-5-methyl-6-oxo-7H-pteridin-2-yl)amino]-N-[22[2-[2-[2-[2-(2-hydroxyethoxy)ethoxy]ethoxy]ethoxy]ethoxy]ethoxy]ethyl]-3-methoxy-benzamide (107 mg, 146 μmol, 1.0 equiv) in DCM (2 mL) was added DMP (124 mg, 292 mol, 1.5 equiv), and it was stirred at 20° C. for 1 h. 4-[(8-cyclopentyl-7-ethyl-5-methyl-6-oxo-7H-pteridin-2-yl)amino]-3-methoxy-N-[2-[2-[2-[2-[2-[2-(2-oxoethoxy)ethoxy]ethoxy]ethoxy]ethoxy]ethoxy]ethyl]benzamide (100 mg, crude) was afford in solution as a yellow liquid. LC-MS: MS (ES⁺): RT=0.810 min, m/z=731.7 [M+H⁺].

### 65.3 Preparation of Compound I-65

To a solution of4-[(8-cyclopentyl-7-ethyl-5-methyl-6-oxo-7H-pteridin-2-yl)amino]-3-methoxy-N-[2-[2-[2-[2-[2-[2-(2-oxoethoxy)ethoxy]ethoxy]ethoxy]ethoxy]ethoxy]ethyl]benzamide (100 mg, 137 μmol, 1.0 equiv) and 2-[(2S)-4-[7-(8-chloro-1-naphthyl)-2-[[(2S)-pyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (77 mg, 0.11 mmol, 0.8 equiv, TFA salt) in DCM (2 mL) and DMF (0.5 mL) was added Et₃N (138 mg, 1.37 mmol, 190 μL, 10.0 equiv) and NaBH(OAc)₃ (290 mg, 1.37 mmol, 10.0 equiv), and it was stirred at 20° C. for 1 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by preparative TLC on silica gel (DCM: MeOH=3:1) to afford a crude product, and then it was purified by prep-HPLC (column: Phenomenex Gemini-NX C18 75*30 mm*3 um; mobile phase: [water(0.225% FA)-ACN]; B%: 25%-55%, 7 min) to afford N-[2-[2-[2-[2-[2-[2-[2-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido1[3,4-d]pyrimidin-2-yl]oxymethyl]pyrrolidin-1-yl]ethoxy]ethoxy]ethoxy]ethoxy]ethoxy]ethoxy]ethyl]-4-[1[(7R)-8-cyclopentyl-7-ethyl-5-methyl-6-oxo-7H-pteridin-2-yl]amino]-3-methoxy-benzamide (14 mg, 10 μmol, 8% yield, FA salt) as a yellow solid. LC-MS: MS (ES⁺): RT=2.131 min, m/z=1304.6 [M+H⁺]. ¹H NMR (400 MHz, CD₃OD) δ8.49 (d, J=8.4 Hz, 1H), 7.87-7.61 (m, 3H), 7.55-7.42 (m, 4H), 7.40-7.24 (m, 2H), 5.44-5.23 (m, 2H), 4.64-4.36 (m, 3H), 4.35-4.03 (m, 6H), 4.02-3.94 (m, 3H), 3.81-3.44 (m, 33H), 3.27-2.50 (m, 11H), 2.20-1.64 (m, 15H), 0.84 (t, J=7.6 Hz, 3H).

### Example 66—Synthesis of Additional Compounds

Additional compounds set forth in the table below were prepared based on procedures described herein. Physical characterization data is also provided in the table, where retention time refers to retention time in the LC-MS analysis.

TABLE 2

| | | | | Retention | |
|---|---|---|---|---|---|
| Compound | | | Observ. | Time | LCMS |
| No. | Structure | MW Exact | Mass | (min) | Method |
| I-66 | 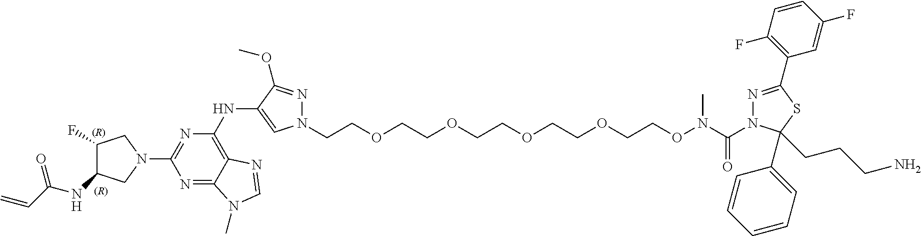 | 1009.4 | 505.8 | 220-2.332 | 10 |
| I-67 | 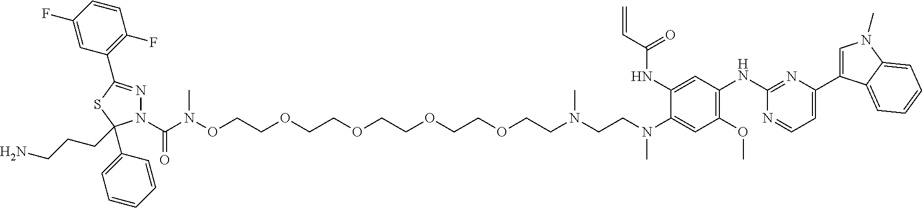 [SMILES: C=CC(=O)Nc1cc(Nc2nccc(-c3cn(C)c4ccccc34)n2)c(OC)cc1N(C)CCN(C)CCOCCOCCOCCOCCON(C)C(=O)N1N=C(c2cc(F)ccc2F)SC1(CCCN)c1ccccc1] | 1093.5 | 547.8, 1094.5 | 220-2.254 | 10 |
| I-68 | 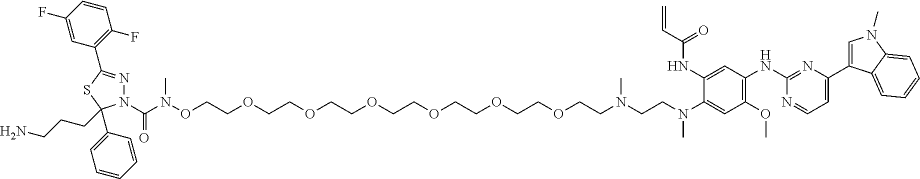 [SMILES: C=CC(=O)Nc1cc(Nc2nccc(-c3cn(C)c4ccccc34)n2)c(OC)cc1N(C)CCN(C)CCOCCOCCOCCOCCOCCOCCON(C)C(=O)N1N=C(c2cc(F)ccc2F)SC1(CCCN)c1ccccc1] | 1181.6 | 591.9, 1182.6 | 220-1.830 | 25 |
| I-69 | 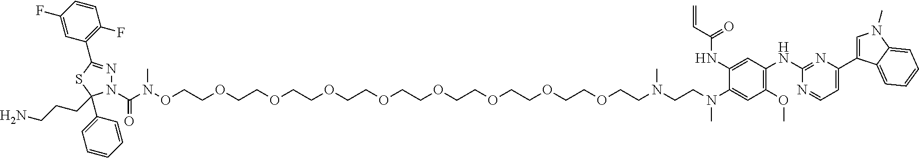 [SMILES: C=CC(=O)Nc1cc(Nc2nccc(-c3cn(C)c4ccccc34)n2)c(OC)cc1N(C)CCN(C)CCOCCOCCOCCOCCOCCOCCOCCOCCON(C)C(=O)N1N=C(c2cc(F)ccc2F)SC1(CCCN)c1ccccc1] | 1269.6 | 635.9, 1270.7 | 220-1.862 | 25 |
| I-70 | 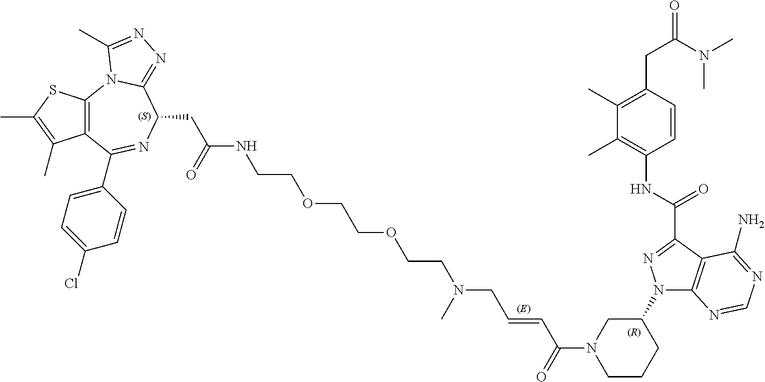 | 1060.5 | 354.7, 531.4 | 220-0.775 | 1 |
| I-71 | 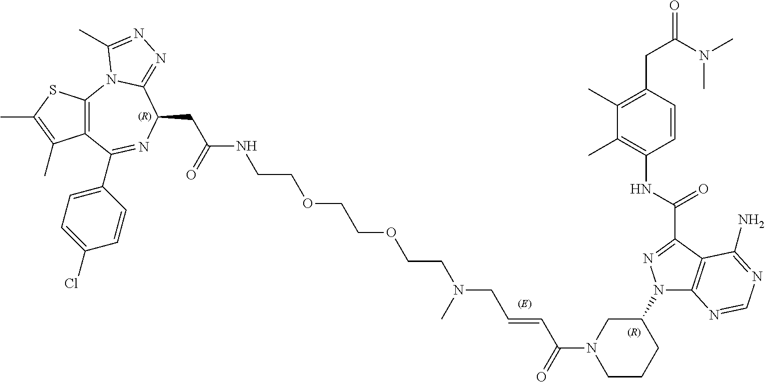 | 1060.5 | 531.2, 1061.3 | 220-2.358 | 10 |
| I-72 | 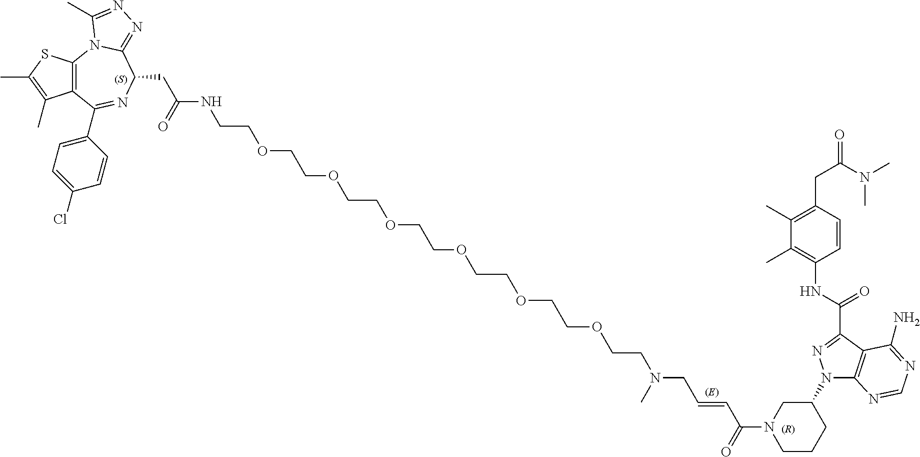 | 1236.6 | 619.4 | | 01 |
| I-73 | 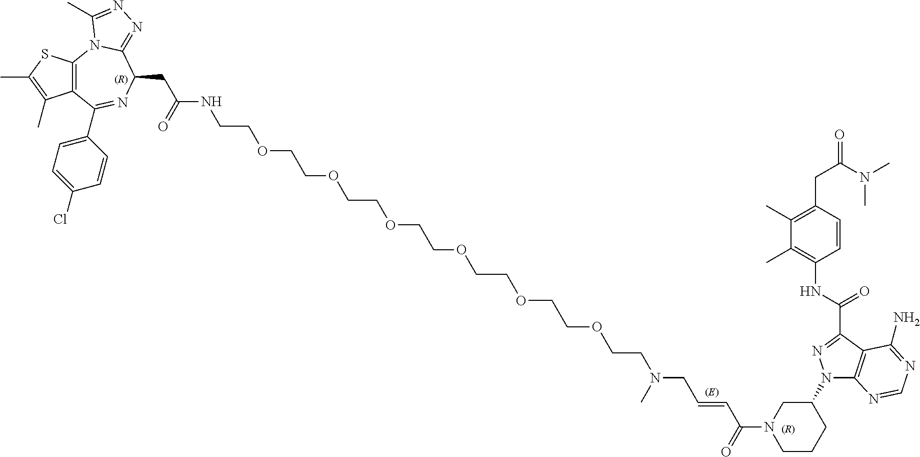 | 1236.6 | 619.3 | | 01 |
| I-74 | 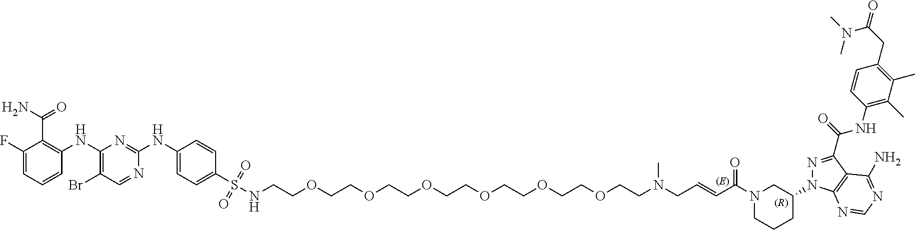 | 1317.5 | 440.8, 660.8 | 220-2.373 | 10 |
| I-75 | 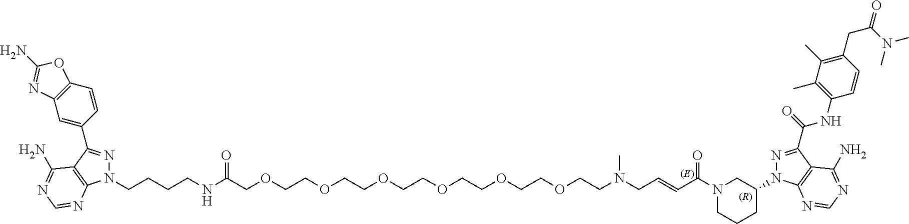 | 1189.6 | 595.8, 1190.5 | 220-2.324 | 01 |
| I-76 | 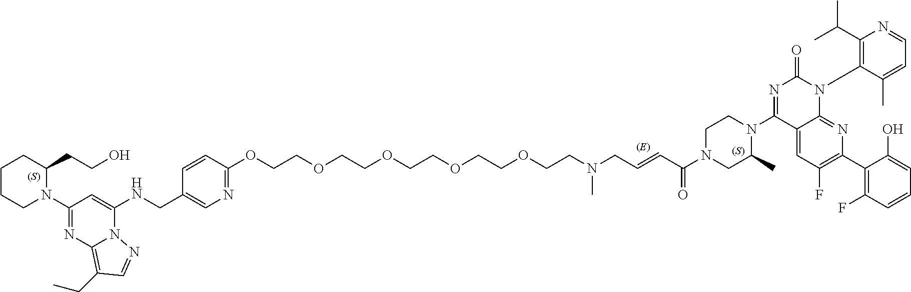 | 1201.6 | 601.9, 1202.7 | 220-1.613 | 25 |
| I-77 | 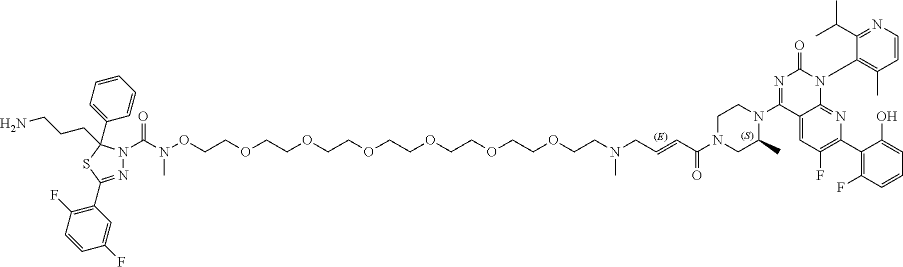 | 1299.6 | 434.3, 650.8 | 220-2.529 | 01 |
| I-78 | 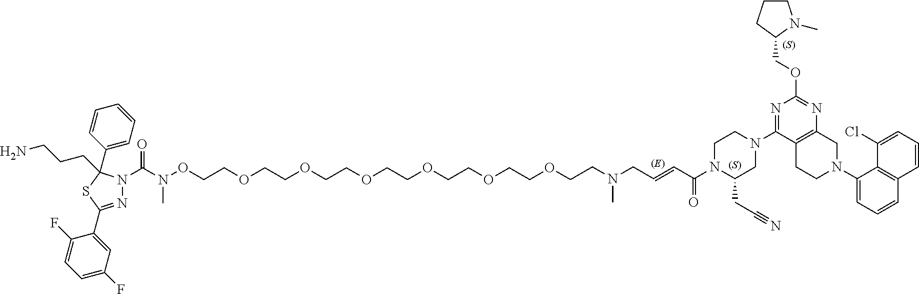 | 1324.6 | 442.7, 663.4 | 220-1.844 | 25 |
| I-79 | 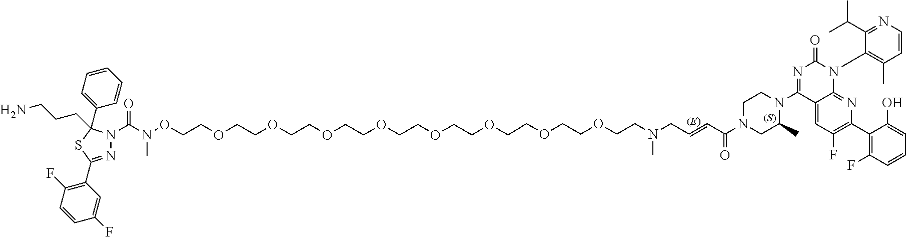 | 1387.6 | 463.6, 694.8 | 220-1.787 | 25 |
| I-80 | 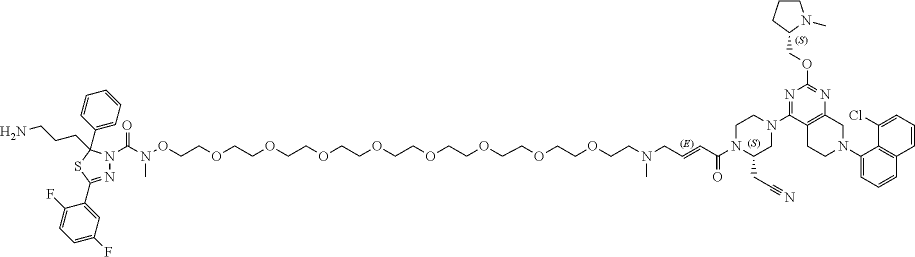 | 1412.7 | 472.1, 707.4 | 220-1.879 | 25 |
| I-81 | 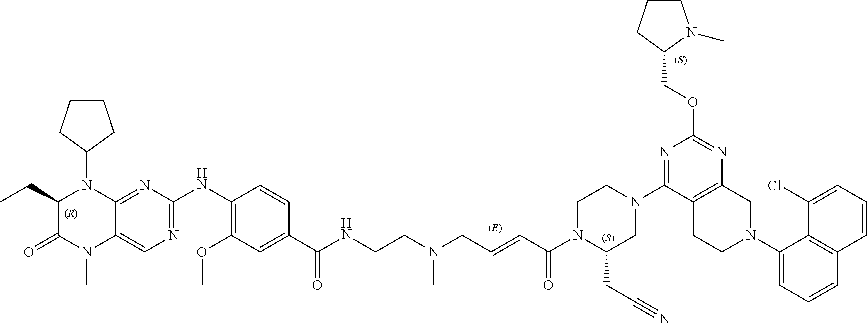 | 1078.5 | 540.3, 1079.4 | 220-1.590 | 25 |
| I-82 | 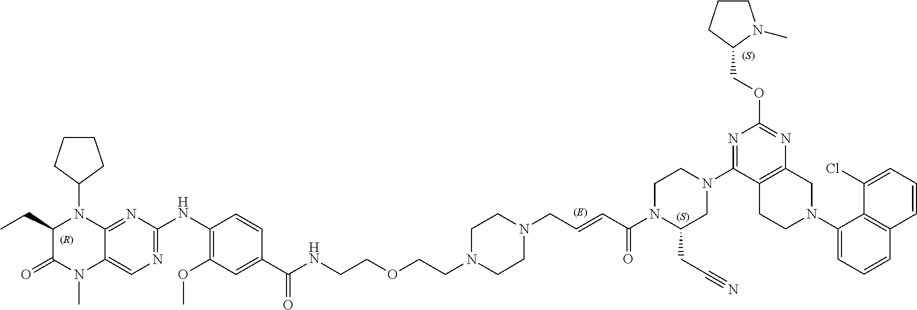 | 1177.6 | 589.9 | 220-1.649 | 25 |

### Example 67—Synthesis of Compound I-83

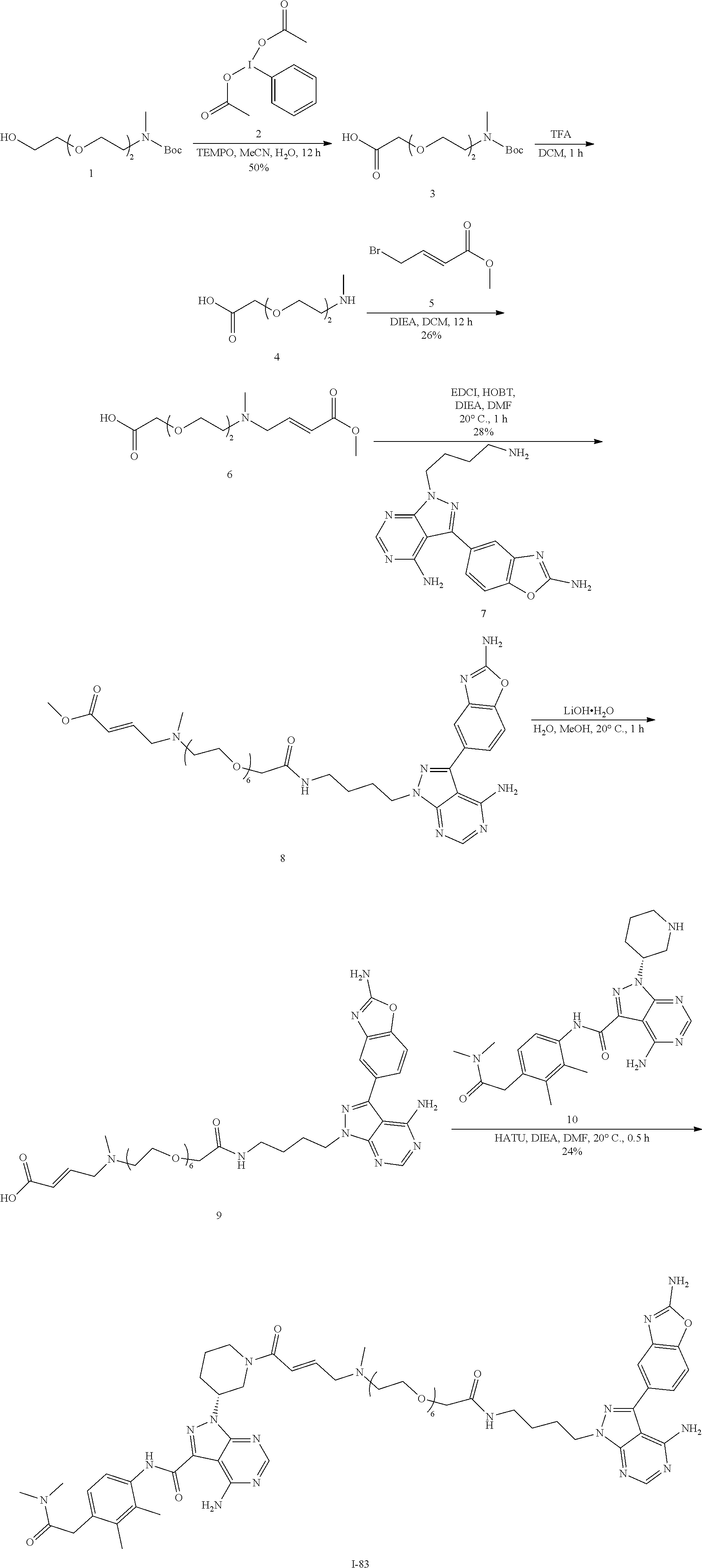

General Information: Compound 1 can be prepared according to procedures described in WO 2013/116682.

Step 1. To a solution of tert-butyl N-[2-[2-(2-hydroxyethoxy)ethoxy]ethyl]-N-methyl-carbamate (1.5 g, 5.7 mmol, 1.0 equiv) in MeCN (15 mL) and H₂O (15 mL) was added TEMPO (197 mg, 1.3 mmol, 0.2 equiv) and [acetoxy(phenyl)-k3-iodanyl]acetate (4.0 g, 12.5 mmol, 2.2 equiv) at 0° C. The mixture was stirred at 20° C. for 12 h. The reaction mixture was purified by column chromatography (SiO₂, Petroleum ether/Ethyl acetate=10/1 to 0/1) to give the desired product 2-[2-[2-[tert-butoxycarbonyl(methyl)amino]ethoxy]ethoxy]acetic acid (0.8 g, 50% yield) as a yellow oil. ¹H NMR (400 MHz, CDCl₃): δ 4.18 (s, 2H), 3.80-3.75 (m, 2H), 3.69 (d, J=4.8 Hz, 4H), 3.45-3.43 (m, 1H), 2.93 (s, 3H), 1.48 (s, 9H).

Step 2. To a solution 2-[2-[2-[tert-butoxycarbonyl(methyl)amino]ethoxy]ethoxy]acetic acid (0.8 g, 2.9 mmol, 1.0 equiv) in DCM (5 mL) was added TFA (2 mL). The mixture was stirred at 20° C. for 1 h. The reaction mixture was concentrated to give the desired product 2-[2-[2-(methylamino)ethoxy]ethoxy]acetic acid (0.8 g, 2.8 mmol, TFA salt) as a yellow oil.

Step 3. To a solution of 2-[2-[2-(methylamino)ethoxy]ethoxy]acetic acid (0.8 g, 2.7 mmol, 1.0 equiv, TFA salt) in DCM (10 mL) was added DIEA (1.0 g, 8.2 mmol, 1.4 mL, 3.0 equiv) and methyl (E)-4-bromobut-2-enoate (492 mg, 2.7 mmol, 323 μL, 1.0 equiv). The mixture was stirred at 20° C. for 12 h. The solution was purified by prep-HPLC (column: Shim-pack C18 150*25*10 μm; mobile phase: [water(0.1%TFA)-ACN]; B%: 1%-25%, 10 min) to give the desired product 2-[2-[2-[[(E)-4-methoxy-4-oxo-but-2-enyl]-methyl-amino]ethoxy]ethoxy]acetic acid (200 mg, 26% yield) as a yellow gum. ¹H NMR (400 MHz, CDCl₃): δ 9.45 (s, 1H) 6.86-6.82 (m, 1H), 5.97 (d, J=15.6 Hz, 1H), 4.19 (s, 2H), 3.84-3.78 (m, 2H), 3.72-3.65 (m, 7H), 3.64-3.61 (m, 2H), 3.17-3.13 (m, 2H), 2.71 (s, 3H).

Step 4. To a solution of 5-[4-amino-1-(4-aminobutyl)pyrazolo[3,4-d]pyrimidin-3-yl]-1,3-benzoxazol-2-amine (160 mg, 353 μmol, 1 equiv, TFA) and 2-[2-[2-[[(E)-4-methoxy-4-oxo-but-2-enyl]-methyl-amino]ethoxy]ethoxy]acetic acid (97 mg, 353 μmol, 1 equiv) in DMF (2 mL) was added DIEA (228 mg, 1.77 mmol, 308 μL, 5 equiv), EDCI (74 mg, 389 pmol, 1.1 equiv) and HOBt (52 mg, 389 μmol, 1.1 equiv). The mixture was stirred at 20° C. for 1 h. The reaction mixture was filtered and concentrated under reduced pressure to give a residue. The residue was purified by prep-HPLC (column: Phenomenex Gemini-NX C18 75*30 mm*3 m; mobile phase: [water (10 mM NH₄HCO₃) -ACN]; B%: 8% -38%, 8 min) to give the desired compound methyl (E)-4-[2-[2-[2-[4[44-amino-3-(2-amino-1,3-benzoxazol-5-yl)pyrazolo[3,4-d]pyrimidin-1-yl]butylamino]-2-oxo-ethoxy]ethoxy]ethyl-methyl-amino]but-2-enoate (60 mg, 28% yield) as a white solid.

Step 5. To a solution of methyl (E)-4-[2-[2-[2-[4-[4-amino-3-(2-amino-1,3-benzoxazol-5-yl)pyrazolo[3,4-d]pyrimidin-1-yl]butylamino]-2-oxo-ethoxy]ethoxy]ethyl-methyl-amino]but-2-enoate (55 mg, 92 μmol, 1 equiv) in H₂O (0.5 mL) and MeOH (2 mL) was added LiOH.H₂O (11 mg, 277 μmol, 3 equiv). The mixture was stirred at 20° C. for 1 h. The reaction mixture was filtered and concentrated under reduced pressure to give the desired compound (E)-4-[2-[2-[2-[4-[4-amino-3-(2-amino-1,3-benzoxazol-5-yl)pyrazolo[3,4-d]pyrimidin-1-yl]butylamino]-2-oxo-ethoxy]ethoxy]ethyl-methyl-amino]but-2-enoic acid (53 mg, crude) as a white solid.

Step 6. To a solution of (E)-4-[2-[2-[2-[4-[4-amino-3-(2-amino-1,3-benzoxazol-5-yl)pyrazolo[3,4-d]pyrimidin-1-yl]butylamino]-2-oxo-ethoxy]ethoxy]ethyl-methyl-amino]but-2-enoic acid (50 mg, 85 mmol, 1 equiv) and 4-amino-N-[4-[2-(dimethylamino)-2-oxo-ethyl]-2,3-dimethyl-phenyl]-1-[(3R)-3-piperidyl]pyrazolo[3,4-d]pyrimidine-3-carboxamide (48 mg, 85 μmol, 1 equiv, TFA) in DMF (2 mL) was added DIEA (55 mg, 429 μmol, 74 mL, 5 equiv) and HATU (32 mg, 85 μmol, 1 equiv). The mixture was stirred at 20° C. for 0.5 h. The reaction mixture was filtered and concentrated under reduced pressure to give a residue. The residue was purified by prep-HPLC (column: 3_Phenomenex Luna C18 75*30 mm*3 m; mobile phase: [water (0.05%HCl)-ACN]; B%: 12%-32%, 6.5 min) to give the compound 4-amino-1-[(3R)-1-[(E)-4-[2-1[2-[2-[4-[4-amino-3-(2-amino-1,3-benzoxazol-5-yl)pyrazolo[3,4-d]pyrimidin-1-yl]butylamino]-2-oxo-ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (23 mg, 24% yield, 4HCl) as a white solid. ¹H NMR (400 MHz, CD₃OD) δ8.60-8.38 (m, 2H), 7.85-7.63 (m, 3H), 7.31 (d, J=7.6 Hz, 1H), 7.18-6.96 (m, 2H), 6.86-6.68 (m, 1H), 4.76-4.26 (m, 4H), 4.15-3.66 (m, 14H), 3.59-3.43 (m, 2H), 3.31-2.82 (m, 12H), 2.45-2.25 (m, 8H), 2.15-1.95 (m, 3H), 1.80 (s, 1H), 1.59-1.57 (m, 2H). LC-MS: MS (ES⁺): RT=2.218 min, m/z=1014.3 [M+H⁺]. LCMS method: 01.

### Example 68—Synthesis of Compound I-84

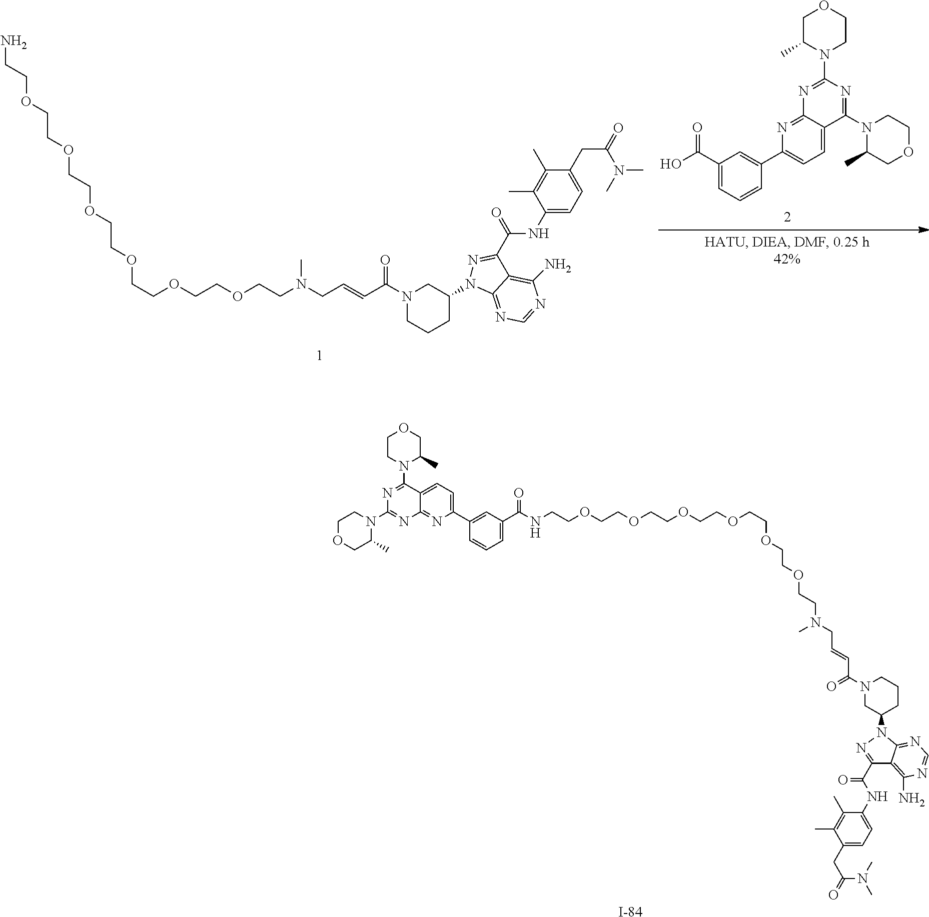

To a solution of 4-amino-1-[(3R)-1-[(E)-4-[2-[2-[2-[2-[2-[2-(2-aminoethoxy)ethoxy]ethoxy]ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (86 mg, 89 μmol, 1.0 equiv, TFA salt), DIEA (35 mg, 267 μmol, 3.0 equiv) in DMF (2 mL) was added HATU (40 mg, 106 μmol, 1.2 equiv) and 3-[2,4-bis[(3R)-3-methylmorpholin-4-yl]pyrido[2,3-d]pyrimidin-7-yl]benzoic acid (40 mg, 89 μmol, 1.0 equiv). The mixture was stirred at 20° C. for 0.25 h. The solution was purified by prep-HPLC (column: Waters Xbridge 150*25 mm*5 m; mobile phase: [water (10 mM NH₄HCO₃)-ACN]; B%: 30%-63%, 9 min) to give 4-amino-[(3R)-1-[(E)-4-[2-[2-[2-[2-[2-[2-[2-[[3-[2,4-bis[(3R)-3-methylmorpholin-4-yl]pyrido[2,3-d]pyrimidin-7-yl]benzoyl]amino]ethoxy]ethoxy]ethoxy]ethoxy]ethoxy]ethoxy]ethyl-methyl-amino]but-2-enoyl]-3-piperidyl]-N-[4-[2-(dimethylamino)-2-oxo-ethyl]-2,3-dimethyl-phenyl]pyrazolo[3,4-d]pyrimidine-3-carboxamide (50 mg, 42% yield). ¹H NMR (400 MHz, DMSO-d₆): δ10.27-10.01 (m, 1H), 8.73 (t, 1H, J=5.6 Hz), 8.67-8.56 (m, 2H), 8.34-8.21 (m, 3H), 8.08 (s, 1H), 7.97 (d, 1H, J=7.6 Hz), 7.71 (d, 1H, J=8.4 Hz), 7.62 (t, 1H, J=7.6 Hz), 7.16 (s, 1H), 6.96 (d, 1H, J=8.0 Hz), 6.72-6.44 (m, 2H), 4.78 (s, 2H), 4.60-4.19 (m, 3H), 4.08 (s, 1H), 3.97-3.88 (m, 3H), 3.77-3.43 (m, 37H), 3.26-2.96 (m, 8H), 2.87 (s, 3H), 2.29-1.90 (m, 12H), 1.59 (s, 1H), 1.38 (d, 3H, J=6.8 Hz), 1.25 (d, 3H, J=6.8 Hz). LC-MS: MS (ES′): RT=2.493 min, m/z=1286.5 [M+H⁺]; LCMS method: 01.

### Example 69—Synthesis of Compound I-85

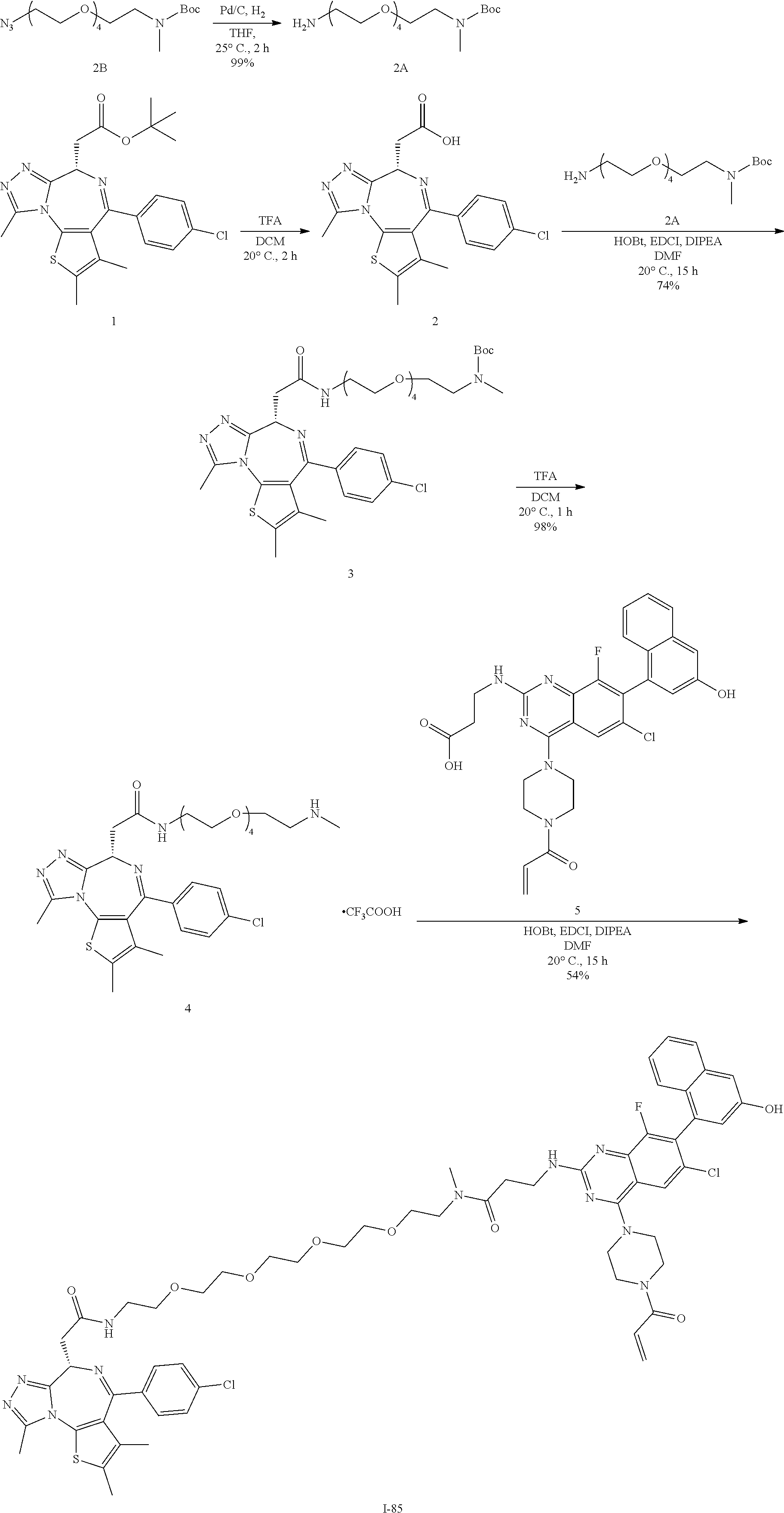

Step 1. To a solution of tert-butyl N-[2-[2-[2-[2-(2-azidoethoxy)ethoxy]ethoxy]ethoxy]ethyl]-N-methyl-carbamate (130 mg, 0.34 mmol, 1.0 equiv) in THF (10 mL) was added wet Pd/C (20 mg, 10% purity). The mixture was stirred at 25° C. for 2 h under H₂ (15 psi). The reaction mixture was filtered and the filtrate was concentrated in vacuo to afford tert-butyl N-[2-[2-[2-[2-(2-aminoethoxy)ethoxy]ethoxy]ethoxy]ethyl]-N-methyl-carbamate (120 mg, 0.34 mmol, 99% yield) as a yellow oil. ¹H NMR (400 MHz, DMSO-d₆): δ3.51-3.43 (m, 14H), 3.35-3.27 (m, 6H), 2.81-2.78 (m, 3H), 1.38 (s, 9H).

Step 2. A mixture of tert-butyl 2-[7-(4-chlorophenyl)-4,5,13-trimethyl-3-thia-1,8,11,12-tetrazatricyclo[8.3.0.02,6]trideca-2(6), 4,7,10,12-pentaen-9-yl]acetate (150 mg, 0.33 mmol, 1.0 equiv) in DCM (2 mL) and TFA (1 mL) was stirred at 20° C. for 2 h. The reaction mixture was concentrated in vacuo to afford 2-[7-(4-chlorophenyl)-4,5,13-trimethyl-3-thia-1,8,11,12-tetrazatricyclo[8.3.0.02,6]trideca-2(6), 4,7,10,12-pentaen-9-yl]acetic acid (132 mg, crude) as a yellow oil.

Step 3. A mixture of 2-[7-(4-chlorophenyl)-4,5,13-trimethyl-3-thia-1,8,11,12-tetrazatricyclo[8.3.0.02,6]trideca-2(6), 4,7,10,12-pentaen-9-yl]acetic acid (132 mg, 0.33 mmol, 1.0 equiv), tert-butyl N-[2-[2-[2-[2-(2-aminoethoxy)ethoxy]ethoxy]ethoxy]ethyl]-N-methylcarbamate (115 mg, 0.33 mmol, 1.0 equiv), EDCI (158 mg, 0.82 mmol, 2.5 equiv), HOBt (111 mg, 0.82 mmol, 2.5 equiv) and DIPEA (213 mg, 1.65 mmol, 5.0 equiv) in DMF (3 mL) was stirred at 20° C. for 15 h under N₂. The reaction mixture was diluted with water (5 mL) and extracted with DCM/MeOH (10/1, 10 mL*3). The combined organic phase was washed with brine (15 mL), dried over anhydrous Na₂SO₄, filtered and concentrated in vacuo. The residue was purified by prep-TLC on silica gel (Dichloromethane:Methanol=10:1) to afford tert-butyl N-[2-[2[2-[2-[[2-[7-(4-chlorophenyl)-4,5,13-trimethyl-3-thia-1,8,11,12-tetrazatricyclo[8.3.0.02,6]trideca-2(6), 4,7,10,12-pentaen-9-yl]acetyl]amino]ethoxy]ethoxy]ethoxy]ethoxy]ethyl]-N-methylcarbamate (180 mg, 0.24 mmol, 74% yield) as a yellow oil. ¹H NMR (400 MHz, CDCl₃) δ7.45-7.43 (m, 2H), 7.37-7.34 (m, 2H), 7.01-7.00 (m, 1H), 4.74 (t, 1H, J=6.8 Hz), 3.69-3.68 (m, 7H), 3.66-3.58 (m, 9H), 3.41-3.38 (m, 2H), 2.98-2.94 (m, 5H), 2.91-2.88 (m, 5H), 2.43 (s, 3H), 1.69 (s, 3H), 1.45 (s, 9H).

Step 4. A mixture of tert-butyl N-[2-[2-[2-[2-[2-[[2-[7-(4-chlorophenyl)-4,5,13-trimethyl-3-thia-1,8,11,12-tetrazatricyclo[8.3.0.02,6]trideca-2(6), 4,7,10,12-pentaen-9-yl]acetyl]amino]ethoxy]ethoxy]ethoxy]ethoxy]ethyl]-N-methylcarbamate (75.0 mg, 0.10 mmol, 1.0 equiv) in DCM (1.5 mL) and TFA (0.5 mL) was stirred at 20° C. for 1 h. The reaction mixture was concentrated in vacuo to afford 2-[7-(4-chlorophenyl)-4,5,13-trimethyl-3-thia-1,8,11,12-tetrazatricyclo[8.3.0.02,6]trideca-2(6), 4,7,10,12-pentaen-9-yl]-N-[2-2-[2-[2-[2-(methylamino) ethoxy]ethoxy]ethoxy]ethoxy]ethyl]acetamide (75 mg, 0.10 mmol, 98% yield, TFA salt) as a yellow oil.

Step. 5. A mixture of 3-[[6-chloro-8-fluoro-7-(3-hydroxy-1-naphthyl)-4-(4-prop-2-enoylpiperazin-1-yl)quinazolin-2-yl]amino]propanoic acid (40 mg, 72.7 μmol, 1.0 equiv), 2-[7-(4-chlorophenyl)-4,5,13-trimethyl-3-thia-1,8,11,12-tetrazatricyclo[8.3.0.02,6]trideca-2(6), 4,7,10,12-pentaen-9-yl]-N-[2-[2-[2-[2-[2-(methylamino)ethoxy]ethoxy]ethoxy]ethoxy]ethyl]acetamide (65.2 mg, 87.3 μmol, 1.2 equiv, TFA salt), HOBt (24.6 mg, 182 μmol, 2.5 equiv), EDCI (34.9 mg, 182 μmol, 2.5 equiv) and DIPEA (47.0 mg, 364 μmol, 5.0 equiv) in DMF (2 mL) was stirred at 20° C. for 15 h under N₂. The reaction mixture was diluted with water (5 mL) and extracted with ethyl acetate (10 mL*3). The combined organic phase was washed with brine (15 mL), dried over anhydrous Na₂SO₄, filtered and concentrated in vacuo. The residue was purified by prep-HPLC (FA condition:column: Phenomenex Luna C18 150*25 mm*10 μm; mobile phase: [water(0.225% FA)-ACN]; B%: 24%-54%, 10 min) to afford 3-[[6-chloro-8-fluoro-7-(3-hydroxy-1-naphthyl)-4-(4-prop-2-enoylpiperazin-1-yl)quinazolin-2-yl]amino]-N-[2-[2-[2-[2-[2-[[2-[7-(4-chlorophenyl)-4,5,13-trimethyl-3-thia-1,8,11,12-tetrazatricyclo[8.3.0.02,6]trideca-2(6), 4,7,10,12-pentaen-9-yl]acetyl]amino]ethoxy]ethoxy]ethoxy]ethoxy]ethyl]-N-methyl-propanamide (46.4 mg, 39.2 μmol, 54% yield, 98.6% purity) as a off-white solid. ¹H NMR (400 MHz, CDCl₃) δ7.74-7.67 (m, 2H), 7.43-7.38 (m, 8H), 7.22-7.20 (m, 1H), 6.67-6.58 (m, 1H), 6.40-6.36 (m, 1H), 5.80-5.78 (m, 1H), 4.76-4.70 (m, 1H), 3.95-3.80 (m, 8H), 3.63-3.43 (m, 23H), 3.11-3.08 (m, 1H), 2.97-2.93 (m, 2H), 2.86-2.76 (m, 1H), 2.70 (s, 3H), 2.40 (s, 3H), 1.71-1.66 (m, 8H). LC-MS: MS (ES′): RT=1.570 min, m/z=1188.3 [M+Na⁺]; LCMS method: 40.

### Example 70—Synthesis of Compound I-86

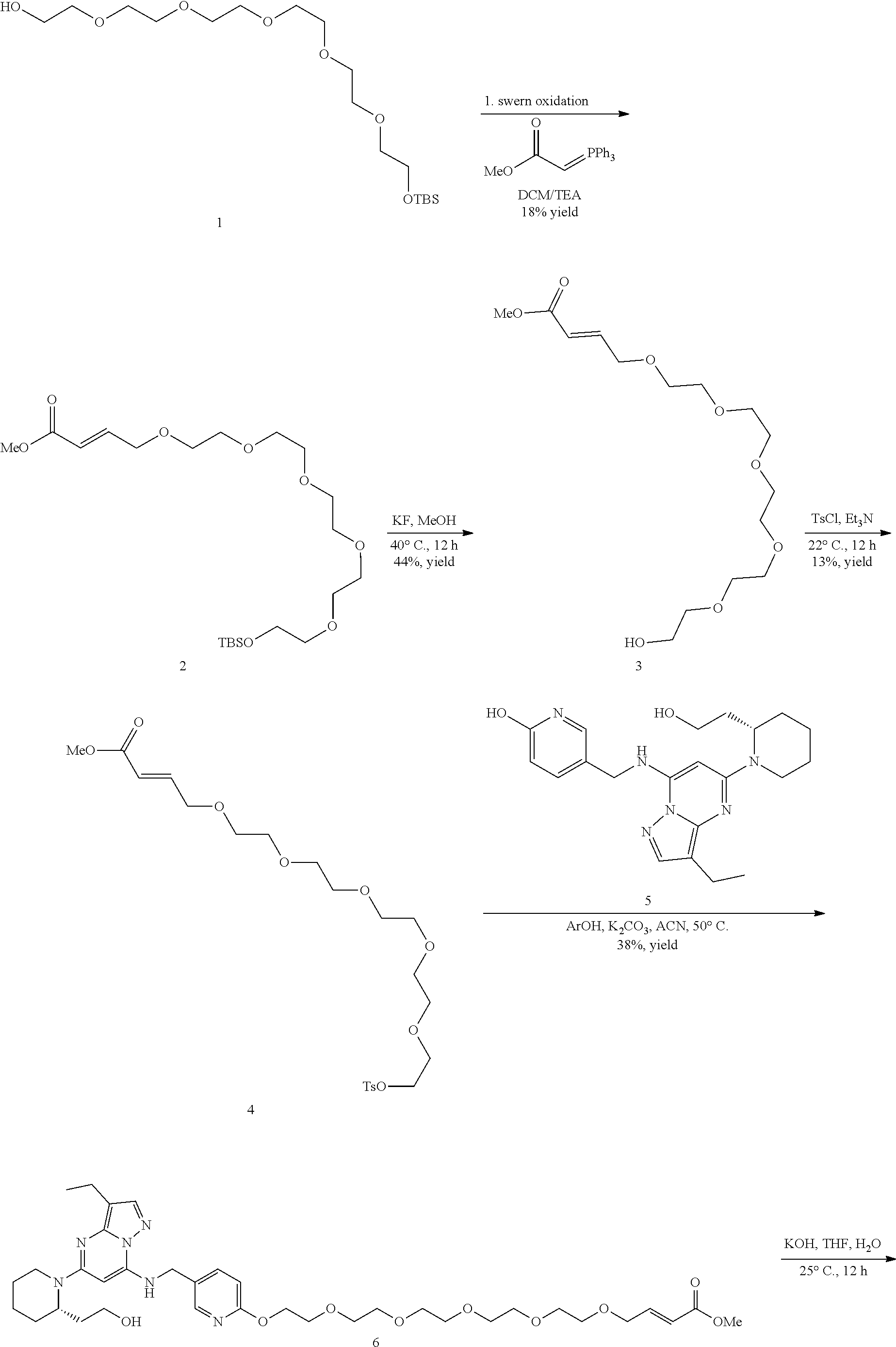

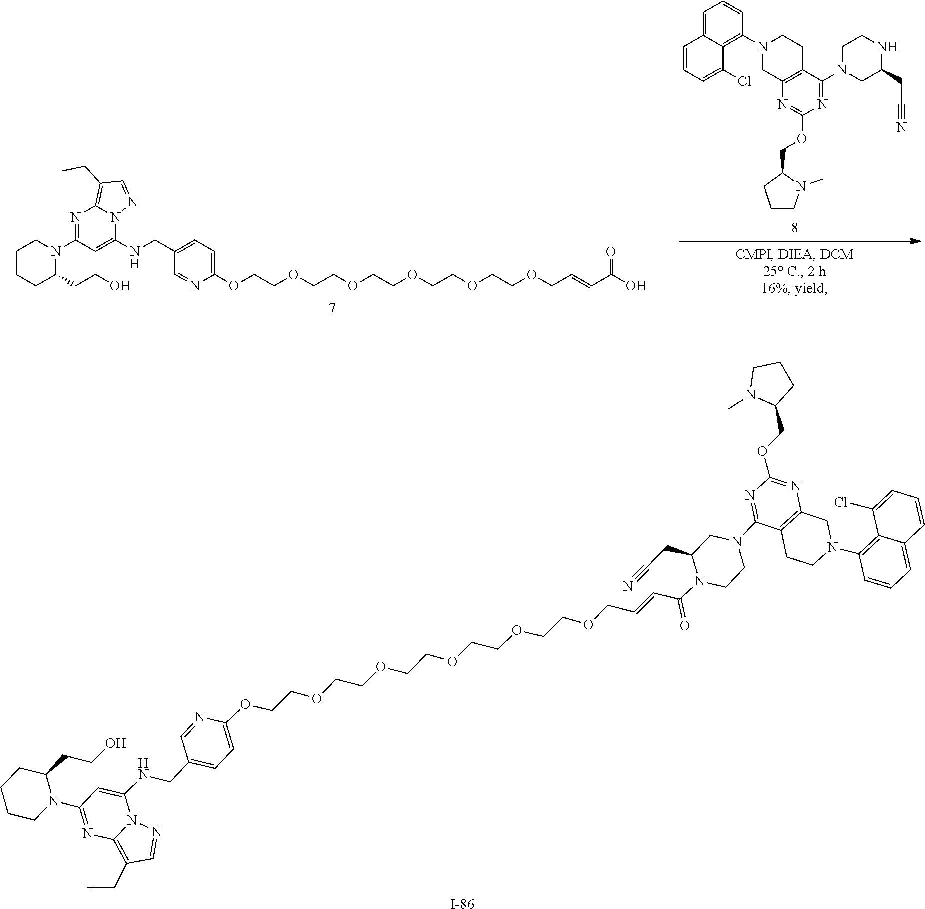

General Information: Synthetic route of compound 8 is reported in WO2021/108683.

Step 1. To a solution of oxalyl dichloride (5.7 g, 44.6 mmol, 3.9 mL, 2.0 equiv) in DCM (120 mL) was added dropwise methylsulfinylmethane (6.9 g, 89.3 mmol, 6.9 mL, 4.0 equiv) at −78° C. The mixture was stirred at −78° C. for 0.5 h. Then to the reaction mixture was added dropwise a solution of 2,2,3,3-tetramethyl-4,7,10,13,16,19-hexaoxa-3-silahenicosan-21-ol (10.8 g, 22.0 mmol, 1.0 equiv) in DCM (30 mL) and the mixture was stirred at −78° C. for 0.5 h. To the reaction mixture was added dropwise TEA (18.0 g, 119 mmol, 15.2 mL, 8.0 equiv) at −78° C. under N₂ for 0.5 h. Then the resulting mixture was stirred at 22° C. under N₂ for 2 h. The reaction mixture was added methyl 2-(triphenyl-k5-phosphanylidene)acetate (10.5 g, 31.5 mmol, 1.5 equiv) in DCM (120 mL). Then the resulting mixture was stirred at 23° C. under N₂ for 12 h. The reaction mixture was filtered and concentrated under reduced pressure to give a residue. The crude product was purified by reversed-phase HPLC (column: Phenomenex luna C18 250*50 mm*15 m; mobile phase: [water(NH₄HCO₃)-ACN]; B%: 50%-80%, 20 min) to give (E)-methyl 2,2,3,3-tetramethyl-4,7,10,13,16,19-hexaoxa-3-silatricos-21-en-23-oate (1.2 g, 2.7 mmol, 18% yield) as yellow oil.

Step 2. A mixture of methyl (E)-4-[2-[2-[2-[2-[2-[tert-butyl(dimethyl)silyl]oxyethoxy]ethoxy]ethoxy]ethoxy]ethoxy]but-2-enoate (1.5 g, 3.3 mmol, 1.0 equiv), KF (966 mg, 16.6 mmol, 389 μL, 5.0 equiv) in MeOH (10 mL) was degassed and purged with N₂ for 3 times, and then the mixture was stirred at 40° C. for 12 h under N₂ atmosphere. The crude reaction mixture was concentrated under reduced pressure to remove solvent. The residue was diluted with H₂O (30 mL) and extracted with DCM (30 mL×3). The combined organic layers were washed with brine (50 mL×2), dried over Na₂SO₄, filtered and concentrated under reduced pressure to give a residue and was purified by prep-HPLC (column: YMC Triart C18 250*50 mm*7 m; mobile phase: [water(NH₄HCO₃)-ACN]; B%: 23%-53%, 10 min) to give the methyl (E)-4-[2-[2-[2-[2-(2-hydroxyethoxy)ethoxy]ethoxy]ethoxy]ethoxy]but-2-enoate (500 mg, 1.49 mmol, 44% yield) as a colorless oil. ¹H NMR: (400 MHz, CDCl₃) δ=6.07 (d, J=16 Hz, 1H), 6.06-6.03 (m, 1H), 4.55-4.43 (m, 1H), 4.22-4.12 (m, 1H), 3.88-3.82 (m, 2H), 3.63-3.56 (m, 20H), 3.15-3.04 (m, 2H).

Step 3. A mixture of methyl (E)-4-[2-[2-[2-[2-(2-hydroxyethoxy) ethoxy]ethoxy]ethoxy]ethoxy]but-2-enoate (400 mg, 1.2 mmol, 1.0 equiv), TosCl (453 mg, 2.4 mmol, 2.0 equiv), TEA (360 mg, 3.6 mmol, 496 μL, 3.0 equiv) in DCM (5 mL) was degassed and purged with N₂ for 3 times, and then the mixture was stirred at 25° C. for 12 h under N₂ atmosphere. The mixture was concentrated to get the residue. The residue was purified by the prep-TLC (Petroleum ether: Ethyl acetate=1:1) give methyl (E)-4-[2-[2-[2-[2-[2-(p-tolylsulfonyloxy)ethoxy]ethoxy]ethoxy]ethoxy]ethoxy]but-2-enoate (300 mg, 611 μmol, 51% yield) as a colorless oil.

Step 4. A mixture of 5-[[[3-ethyl-5-[2-(2-hydroxyethyl)-1-piperidyl]pyrazolo[1,5-a]pyrimidin-7-yl]amino]methyl]pyridin-2-ol (20 mg, 50 μmol, 1.0 equiv), methyl (E)-4-[2-[2-[2-[2-[2-(p-tolylsulfonyloxy)ethoxy]ethoxy]ethoxy]ethoxy]ethoxy]but-2-enoate (29 mg, 60 μmol, 1.2 equiv), K₂CO₃ (20 mg, 151 μmol, 3.0 equiv) in DMF (1 mL) was degassed and purged with N₂ for 3 times, and then the mixture was stirred at 50° C. for 12 h under N₂ atmosphere. The mixture was filtered and concentrated to get the residue. The residue was purified by the prep-HPLC (column: Waters x-bridge 150*25 mm*10 m; mobile phase: [water(NH₄HCO₃)-ACN]; B%: 33%-63%, 11 min) to give methyl (E)-4-[2-[2-[2-[2-[2-[[5-[[[3-ethyl-5-[(2S)-2-(2-hydroxyethyl)-1-piperidyl]pyrazolo[ 1,5-a]pyrimidin-7-yl]amino]methyl]-2-pyridyl]oxy]ethoxy]ethoxy]ethoxy]ethoxy]ethoxy]but-2-enoate (70 mg, 97 μmol, 38% yield) as a yellow oil. ¹H NMR: (400 MHz, CDCl₃) δ=8.17 (d, J=2.4 Hz, 1H), 7.65 (s, 1H), 7.64-7.60 (m, 1H), 6.82 (d, J=8.4 Hz, 1H), 6.34-6.25 (m, 1H), 6.17-6.12 (m, 1H), 5.16-5.05 (m, 1H), 4.65-4.55 (m, 1H), 4.54-4.43 (m, 4H), 4.00-3.86 (m, 4H), 3.77-3.64 (m, 20H), 3.42-3.29 (m, 1H), 3.20-3.14 (m, 2H), 3.13-2.99 (m, 1H), 2.73-2.59 (m, 2H), 2.17-2.02 (m, 1H), 1.86-1.66 (m, 6H), 1.29-1.25 (m, 3H).

Step 5. A mixture of methyl (E)-4-[2-[2-[2-[2-[2-[[5-[[[3-ethyl-5-[2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]but-2-enoate (53 mg, 74 μmol, 1.0 equiv), KOH (1 M, 148 μL, 2.0 equiv) in THF (1 mL) was degassed and purged with N₂ for 3 times, and then the mixture was stirred at 25° C. for 1 h under N₂ atmosphere. The mixture was concentrated to get the residue. The residue was used for next step without further purification. Compound (E)-4-[2-[2-[2-[2-[2-[[5-[[[3-ethyl-5-[2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]but-2-enoic acid (50 mg, 71 μmol) was obtained as a white solid.

Step 6. A mixture of (E)-4-[2-[2-[2-[2-[2-[[5-[[[3-ethyl-5-[2-(2-hydroxyethyl)-1-piperidyl]pyrazolo1[1,5-a]pyrimidin-7-yl]amino]methyl]-2-pyridyl]oxy]ethoxy]ethoxy]ethoxy]ethoxy]ethoxy]but-2-enoic acid (50 mg, 71 μmol, 1.0 equiv), 2-[(2S)-4-[7-(8-chloro-1-naphthyl)-2-[[(2S)-1-methylpyrrolidin-2-yl]methoxy]-6,8-dihydro-SH-pyrido[3,4-d]pyrimidin-4-yl]piperazin-2-yl]acetonitrile (45 mg, 85 μmol, 1.2 equiv), HATU (32 mg, 85 μmol, 1.2 equiv), DIPEA (27 mg, 214 μmol, 37 μL, 3.0 equiv) in DMA (2 mL) was degassed and purged with N₂ for 3 times, and then the mixture was stirred at 25° C. for 2 h under N₂ atmosphere. The mixture was filtered and concentrated to get the residue. The residue was purified by the prep-HPLC(column: Waters xbridge 150*25 mm*10 m; mobile phase: [water(NH₄HCO₃)-ACN]; B%: 58%-88%, 8 min) to give the 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-[(E)-4-[2-[2-[2-[2-[2-[[5-[[[3-ethyl-5-[2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]but-2-enoyl]piperazin-2-yl]acetonitrile (15 mg, 11 μmol, 16% yield, 96% purity) as a white solid. LC-MS: MS (ES⁺): RT=2.576 min, m/z=652.1 [1/2M+H⁺]. LCMS Method: 05. ¹H NMR: (400 MHz, MeOD) δ==8.20-8.14 (m, 1H), 7.79 (d, J=7.2 Hz, 1H), 7.75-7.70 (m, 1H), 7.67-7.60 (m, 2H), 7.56-7.20 (m, 5H), 6.77 (d, J=8.4 Hz, 1H), 6.26-6.17 (m, 1H), 5.52 (s, 1H), 4.51 (s, 3H), 4.41-4.33 (m, 5H), 4.12-4.08 (m, 1H), 4.05-3.97 (m, 3H), 3.95-3.89 (m, 2H), 3.83-3.75 (m, 3H), 3.66-3.52 (m, 19H), 3.16-3.13 (m, 3H), 3.03-2.92 (m, 4H), 2.88-2.83 (m, 1H), 2.57-2.48 (m, 6H), 2.14-2.01 (m, 3H), 1.86-1.77 (m, 3H), 1.75-1.62 (m, 8H), 1.33-1.27 (m, 2H), 1.25-1.20 (m, 3H).

### Example 71—Synthesis of Compound I-87

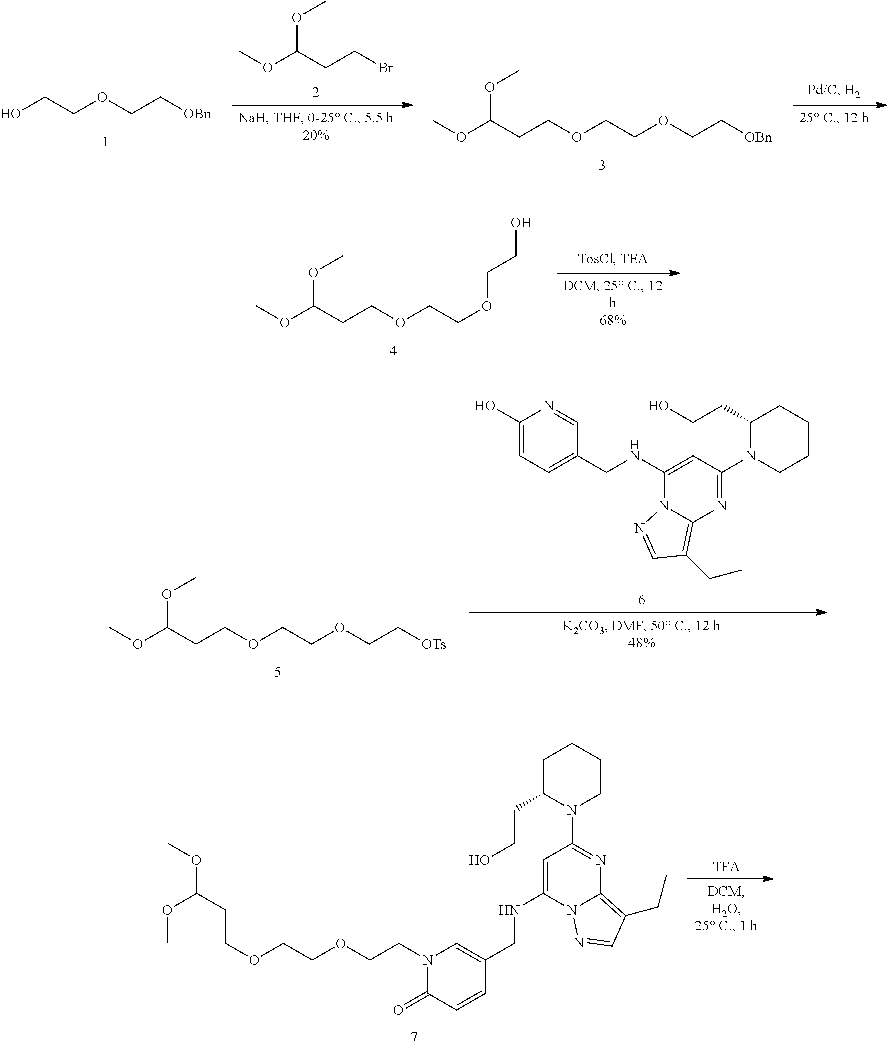

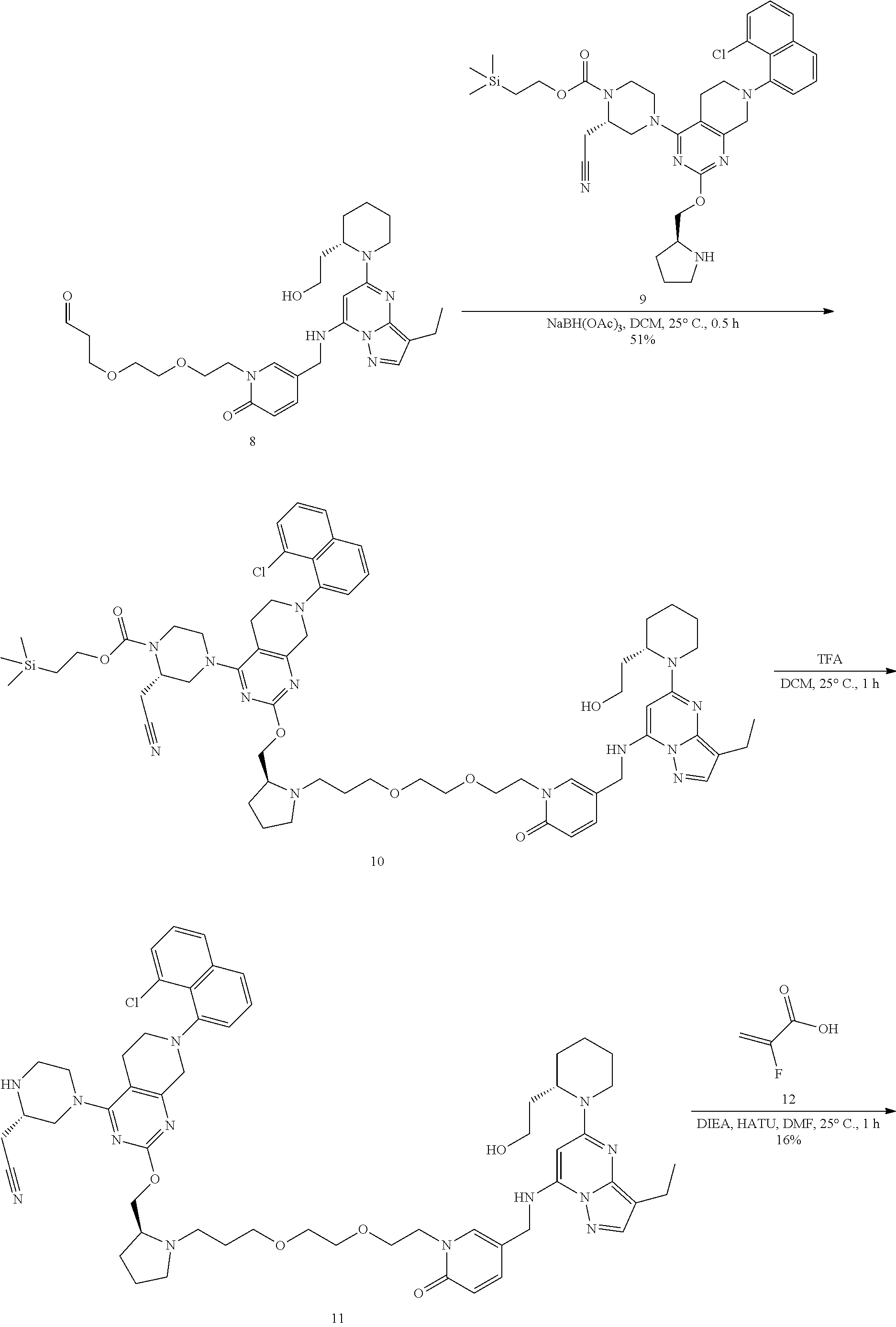

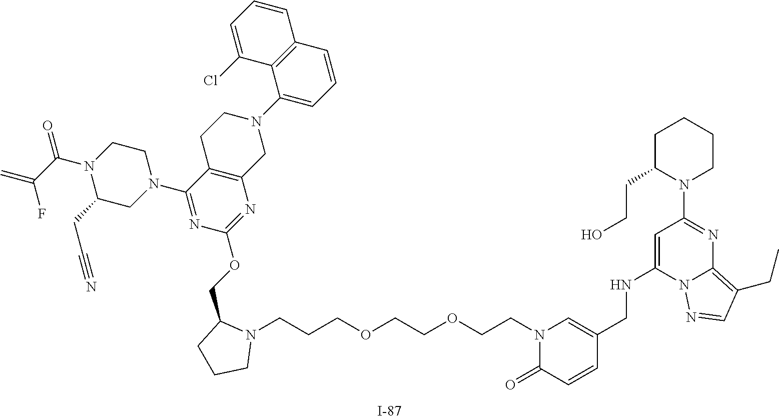

Step 1. To a solution of 2-(2-benzyloxyethoxy)ethanol (1.61 g, 8.19 mmol, 2.68 mL, 1.5 equiv) in THF (20 mL) was added NaH (327. mg, 8.19 mmol, 60% purity, 1.5 equiv) at 0° C., 3-bromo-1,1-dimethoxy-propane (1.00 g, 5.46 mmol, 746 μL, 1.0 equiv) was added and then stirred at 20° C. for 12 h. This reaction was quenched by aq. NH₄Cl (1.0 mL) and concentrated. The residue was purified by prep-HPLC (column: Waters Xbridge C18 150*50 mm*1l0m; mobile phase:[water (NH₄HCO₃)-ACN]; B%: 31%-61%, 11 min) to afford 2-[2-(3,3-dimethoxypropoxy)ethoxy]ethoxymethylbenzene (330 mg, 1.11 mmol, 20% yield) as a colorless oil.

Step 2. To a solution of 2-[2-(3,3-dimethoxypropoxy)ethoxy]ethoxymethylbenzene (165 mg, 553 μmol, 1.0 equiv) in THF (10 mL) was added Pd/C (50 mg, 10% purity) under N₂ atmosphere. The suspension was degassed and purged with H₂ for 3 times. The mixture was stirred under H₂ (15 psi) at 25° C. for 12 h. After filtered, the crude product in filtrate and concentrated. 2-[2-(3,3-dimethoxypropoxy)ethoxy]ethanol (110 mg, 528 μmol) was obtained as a colorless oil and it was used by next step directly.

Step 3. To a solution of 2-[2-(3,3-dimethoxypropoxy)ethoxy]ethanol (110 mg, 528 μmol, 1.0 equiv) in DCM (2 mL) was added TEA (160 mg, 1.58 mmol, 220 μL, 3.0 equiv) and TosCl (201 mg, 1.06 mmol, 2.0 equiv). The mixture was stirred at 25° C. for 12 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The crude product was purified by column chromatography on silica gel (petroleum ether: ethyl acetate=5:1 to 1:) to give 2-[2-(3,3-dimethoxypropoxy)ethoxy]ethyl 4-methylbenzenesulfonate (130 mg, 358 μmol, 68% yield) as a yellow oil.

Step 4. To a solution of 2-[2-(3,3-dimethoxypropoxy)ethoxy]ethyl 4-methylbenzenesulfonate (130 mg, 358 μmol, 1.0 equiv) and 5-[[[3-ethyl-5-[(2S)-2-(2-hydroxyethyl)-1-piperidyl]pyrazolo[1,5-a]pyrimidin-7-yl]amino]methyl]pyridin-2-ol (142 mg, 358 μmol, 1.0 equiv) in DMF (3 mL) was added K₂CO₃ (49.57 mg, 358.6 μmol, 1.0 equiv), the mixture was stirred at 50° C. for 12 h. After filtered, the filtrate was purified by prep-HPLC (column: Waters Xbridge 150*25 mm*5 m; mobile phase: [water(NH₄HCO₃)-ACN]; B%: 45%-75%, 7 min) to afford 1-[2-[2-(3,3-dimethoxypropoxy)ethoxy]ethyl]-5-[[[3-ethyl-5-[(2S)-2-(2-hydroxyethyl)-1-piperidyl]pyrazolo[1,5-a]pyrimidin-7-yl]amino]methyl]pyridin-2-one (100 mg, 17.0 μmol, 48% yield) as a yellow oil.

Step 5. To a solution of 1-[2-[2-(3,3-dimethoxypropoxy)ethoxy]ethyl]-5-[[[3-ethyl-5-[(2S)-2-(2-hydroxyethyl)-1-piperidyl]pyrazolo[ 1,5-a]pyrimidin-7-yl]amino]methyl]pyridin-2-one (80.0 mg, 136 μmol, 1.0 equiv) in DCM (1.5 mL) and H₂O (0.03 mL) was added TFA (0.15 mL). The mixture was stirred at 25° C. for 1 h. The reaction mixture was adjusted with Et₃N to pH=7-8 at 0° C. The crude product 3-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]propanal (72.0 mg, 133 μmol) in DCM was used into the next step without further purification as colorless liquid.

Step 6. To a solution of 2-trimethylsilylethyl (2S)-4-[7-(8-chloro-1-naphthyl)-2-[[(2S)-pyrrolidin-2-yl]methoxy]-6,8-dihydro-SH-pyrido1[3,4-d]pyrimidin-4-yl]-2-(cyanomethyl)piperazine-1-carboxylate (103 mg, 133 μmol, 1.0 equiv, TFA salt) in DCM (2 mL) was added NaBH(OAc)₃ (282 mg, 1.33 mmol, 10.0 equiv) and 3-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]propanal (72.0 mg, 133 μmol, 1.0 equiv). The mixture was stirred at 25° C. for 0.5 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by prep-HPLC (column: 3_Phenomenex Luna C18 75*30 mm*3 m; mobile phase: [water(TFA)-ACN]; B%: 41%-71%, 7 min) to afford 2-trimethylsilylethyl (2S)-4-[7-(8-chloro-1-naphthyl)-2-[[(2S)-1-[3-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]propyl]pyrrolidin-2-yl]methoxy]-6,8-dihydro-5H-pyrido[3,4-d]pyrimidin-4-yl]-2-(cyanomethyl)piperazine-1-carboxylate (80 mg, 67 μmol, 51% yield) as an off-white solid.

Step 7. To a solution of 2-trimethylsilylethyl (2S)-4-[7-(8-chloro-1-naphthyl)-2-[[(2S)-1-[3-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]propyl]pyrrolidin-2-yl]methoxy]-6,8-dihydro-5H-pyrido[3,4-d]pyrimidin-4-yl]-2-(cyanomethyl)piperazine-1-carboxylate (80 mg, 67 μmol, 1.0 equiv) in DCM (1 mL) was added TFA (0.5 mL). The mixture was stirred at 25° C. for 1 h. The reaction mixture was concentrated in vacuum to give 2-[(2S)-4-[7-(8-chloro-1-naphthyl)-2-[[(2S)-1-[3-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]propyl]pyrrolidin-2-yl]methoxy]-6,8-dihydro-5H-pyrido[3,4-d]pyrimidin-4-yl]piperazin-2-yl]acetonitrile (77 mg, 66 μmol, TFA salt) as a yellow oil and it was used by next step directly.

Step 8. To a solution of 2-[(2S)-4-[7-(8-chloro-1-naphthyl)-2-[[(2S)-1-[3-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]propyl]pyrrolidin-2-yl]methoxy]-6,8-dihydro-5H-pyrido[3,4-d]pyrimidin-4-yl]piperazin-2-yl]acetonitrile (77 mg, 66 μmol, 1.0 equiv, TFA salt) and 2-fluoroprop-2-enoic acid (8.99 mg, 99.8 μmol, 1.5 equiv) in DMF (1 mL) was added DIEA (25.8 mg, 199 μmol, 34.7 μL, 3.0 equiv) and HATU (50.62 mg, 133.1 μmol, 2.0 equiv). The mixture was stirred at 25° C. for 1 h. The residue was purified by prep-HPLC (column: Phenomenex C18 75*30 mm*3 m; mobile phase: [water(FA)-ACN]; B%: 22%-52%, 7 min) to afford 2-[(2S)-4-[7-(8-chloro-1-naphthyl)-2-[[(2S)-1-[3-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]propyl]pyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (12.5 mg, 10.5 μmol, 16% yield, 93.66% purity) as a yellow solid. ¹H NMR (400 MHz, CD₃OD) δ8.56-8.42 (m, 1H), 7.81 (d, J=8.0 Hz, 1H), 7.71-7.56 (m, 4H), 7.52-7.42 (m, 2H), 7.38-7.26 (m, 2H), 6.52 (d, J=9.0 Hz, 1H), 5.54 (s, 1H), 5.41-5.23 (m, 2H), 4.50-4.05 (m, 1OH), 3.75-3.35 (m, 16H), 3.25-2.90 (m, 8H), 2.79-2.43 (m, 4H), 2.35-1.58 (m, 14H), 1.56-1.26 (m, 3H), 1.22 (t, J=7.6 Hz, 3H). LC-MS: MS (ES⁺): RT=2.020 min, m/z=557.8 [M/2+H⁺]; LCMS Method: 25.

### Example 72—Synthesis of Compound I-88

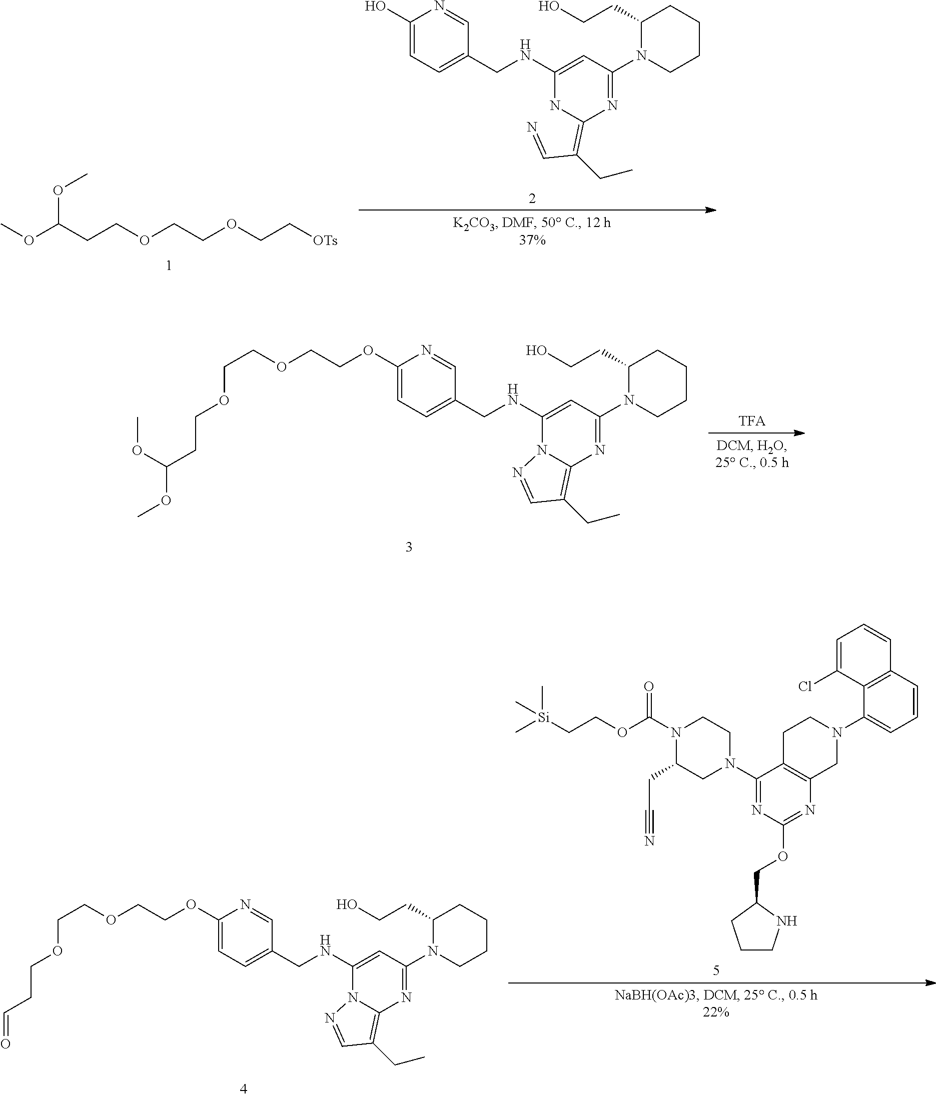

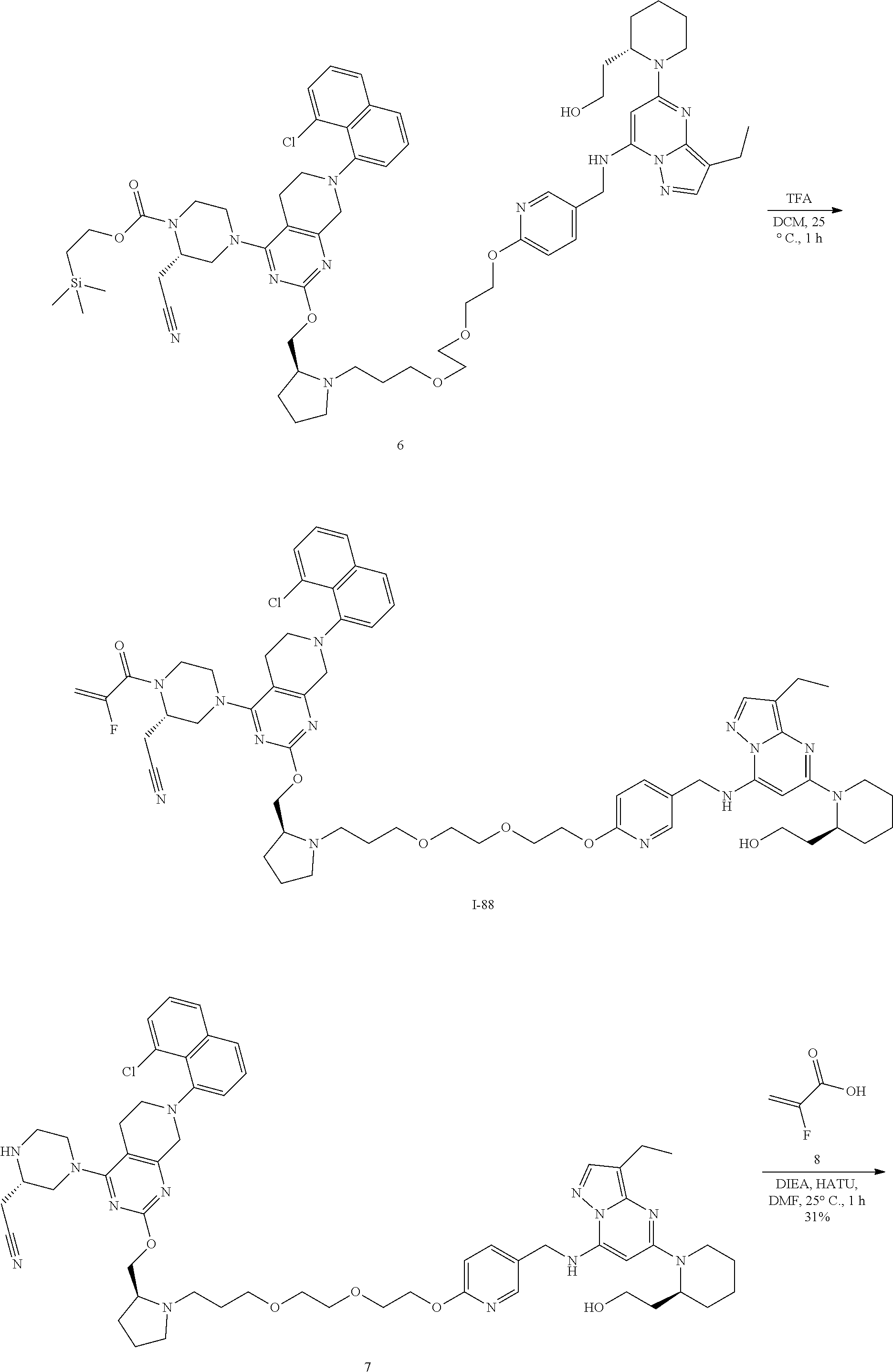

Step 1. To a solution of 2-[2-(3,3-dimethoxypropoxy)ethoxy]ethyl 4-methylbenzenesulfonate (130 mg, 358 μmol, 1.0 equiv) and 5-[[[3-ethyl-5-[(2S)-2-(2-hydroxyethyl)-1-piperidyl]pyrazolo[1,5-a]pyrimidin-7-yl]amino]methyl]pyridin-2-ol (142 mg, 358 μmol, 1.0 equiv) in DMF (3 mL) was added K₂CO₃ (49.57 mg, 358.6 μmol, 1.0 equiv), the mixture was stirred at 50° C. for 12 h. The mixture was filtered and concentrated to give a residue. The residue was purified by prep-HPLC (column: Waters Xbridge 150*25 mm*5 m; mobile phase: [water(NH4HCO₃)-ACN]; B%: 45%-75%, 7 min) to afford 2-[(2S)-1-[7-[[6-[2-[2-(3,3-dimethoxypropoxy)ethoxy]ethoxy]-3-pyridyl]methylamino]-3-ethyl-pyrazolo [1,5-a]pyrimidin-5-yl]-2-piperidyl]ethanol (78 mg, 132.94 μmol, 37.06% yield) as a yellow oil.

Step 2. To a solution of 2-[(2S)-1-[7-[[6-[2-[2-(3,3-dimethoxypropoxy)ethoxy]ethoxy]-3-pyridyl]methylamino]-3-ethyl-pyrazolo[1,5-a]pyrimidin-5-yl]-2-piperidyl]ethanol (68.0 mg, 115 μmol, 1.0 equiv) in DCM (1.5 mL) was added TFA (0.15 mL) and H₂O (0.03 mL). The mixture was stirred at 20° C. for 0.5 h. The reaction mixture was adjusted with Et₃N to pH=7-8 at 0° C. The crude product 3-[2-[2-[[5-[[[3-ethyl-5-[(2S)-2-(2-hydroxyethyl)-1-piperidyl]pyrazolo[ 1,5-a]pyrimidin-7-yl]amino]methyl]-2-pyridyl]oxy]ethoxy]ethoxy]propanal (61.0 mg, 112 μmol) in DCM was used into the next step without further purification as colorless liquid.

Step 3. To a solution of 2-trimethylsilylethyl (2S)-4-[7-(8-chloro-1-naphthyl)-2-[[(2S)-pyrrolidin-2-yl]methoxy]-6,8-dihydro-5H-pyrido[3,4-d]pyrimidin-4-yl]-2-(cyanomethyl) piperazine-1-carboxylate (87.5 mg, 112 μmol, 10.0 equiv, TFA salt) in DCM (2 mL) was added NaBH(OAc)₃ (239 mg, 1.13 mmol, 10.0 equiv) and 3-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]propanal (61.0 mg, 112 μmol, 1.0 equiv). The mixture was stirred at 20° C. for 0.5 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by prep-HPLC (column: 3_Phenomenex Luna C18 75*30 mm*3 m; mobile phase: [water(TFA)-ACN]; B%: 43%-73%, 7 min) to afford 2-trimethylsilylethyl (2S)-4-[7-(8-chloro-1-naphthyl)-2-[[(2S)-1-[3-[2-[2-[[5-[[[3-ethyl-5-[(2S)-2-(2-hydroxyethyl)-1-piperidyl]pyrazolo[ 1,5-a]pyrimidin-7-yl]amino]methyl]-2-pyridyl]oxy]ethoxy]ethoxy]propyl]pyrrolidin-2-yl]methoxy]-6,8-dihydro-5H-pyrido[3,4-d]pyrimidin-4-yl]-2-(cyanomethyl)piperazine-1-carboxylate (30 mg, 25 μmol, 22% yield) as an off-white solid.

Step 4. To a solution of 2-trimethylsilylethyl (2S)-4-[7-(8-chloro-1-naphthyl)-2-[[(2S)-1-[3-[2-[2-[[5-[[[3-ethyl-5-[(2S)-2-(2-hydroxyethyl)-1-piperidyl]pyrazolo [1,5-a]pyrimidin-7-yl]amino]methyl]-2-pyridyl]oxy]ethoxy]ethoxy]propyl]pyrrolidin-2-yl]methoxy]-6,8-dihydro-5H-pyrido[3,4-d]pyrimidin-4-yl]-2-(cyanomethyl) piperazine-1-carboxylate (25 mg, 21 μmol, 1.0 equiv) in DCM (1 mL) was added TFA (0.5 mL). The mixture was stirred at 25° C. for 1 h. After concentrated, the residue was concentrated in vacuum to give 2-[(2S)-4-[7-(8-chloro-1-naphthyl)-2-[[(2S)-1-[3-[2-[2-[[5-[[[3-ethyl-5-[(2S)-2-(2-hydroxyethyl)-1-piperidyl]pyrazolo[ 1,5-a]pyrimidin-7-yl]amino]methyl]-2-pyridyl]oxy]ethoxy]ethoxy]propyl]pyrrolidin-2-yl]methoxy]-6,8-dihydro-5H-pyrido[3,4-d]pyrimidin-4-yl]piperazin-2-yl]acetonitrile (24 mg, 20 μmol, crude, TFA salt) as a yellow oil and it was used by next step.

Step 5. To a solution of 2-[(2S)-4-[7-(8-chloro-1-naphthyl)-2-[[(2S)-1-[3-[2-[2-[[5-[[[3-ethyl-5-[(2S)-2-(2-hydroxyethyl)-1-piperidyl]pyrazolo[ 1,5-a]pyrimidin-7-yl]amino]methyl]-2-pyridyl]oxy]ethoxy]ethoxy]propyl]pyrrolidin-2-yl]methoxy]-6,8-dihydro-5H-pyrido[3,4-d]pyrimidin-4-yl]piperazin-2-yl]acetonitrile (24 mg, 20 μmol, 1.0 equiv, TFA salt) and 2-fluoroprop-2-enoic acid (2.80 mg, 31.1 μmol, 1.5 equiv) in DMF (2 mL) was added DIEA (8.04 mg, 62.2 μmol, 10.8 μL, 3.0 equiv) and HATU (15.78 mg, 41.50 μmol, 2.0 equiv). The residue was purified by prep-HPLC (column: Phenomenex C18 75*30 mm*3 m; mobile phase: [water(FA)-ACN]; B%: 22%-52%, 7 min) to give 2-[(2S)-4-[7-(8-chloro-1-naphthyl)-2-[[(2S)-1-[3-[2-[2-S[[5-[[[3-ethyl-5-[(2S)-2-(2-hydroxyethyl)-1-piperidyl]pyrazolo[ 1,5-a]pyrimidin-7-yl]amino]methyl]-2-pyridyl]oxy]ethoxy]ethoxy]propyl]pyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (7.52 mg, 6.53 μmol, 31% yield, 96.85% purity) as a yellow solid. ¹H NMR (400 MHz, CD₃OD) δ8.48-8.36 (m, 1H), 8.19-8.11 (m, 1H), 7.79 (d, J=8.2 Hz, 1H), 7.73-7.67 (m, 1H), 7.64 (s, 1H), 7.52-7.42 (m, 2H), 7.37-7.24 (m, 2H), 6.72 (m, 1H), 5.52 (d, J=3.3 Hz, 1H), 5.41-5.24 (m, 2H), 4.51 (m, 2H), 4.37-4.33 (m, 2H), 4.24-3.95 (m, 4H), 3.90-3.82 (m, 1H), 3.80-3.46 (m, 14H), 3.43-3.35 (m, 2H), 3.26-3.20 (m, 3H), 3.17-3.07 (m, 3H), 3.01-2.91 (m, 2H), 2.74-2.64 (m, 1H), 2.61-2.50 (m, 2H), 2.34-2.20 (m, 1H), 2.14-1.89 (m, 6H), 1.78-1.56 (m, 6H), 1.53-1.42 (m, 1H), 1.40-1.36 (m, 4H), 1.32-1.28 (m, 1H), 1.25-1.19 (m, 3H). LC-MS: MS (ES′): RT=2.239 min, m/z=557.8 [M/2+H⁺]; LCMS Method: 25.

### Example 73—Synthesis of Compound I-89

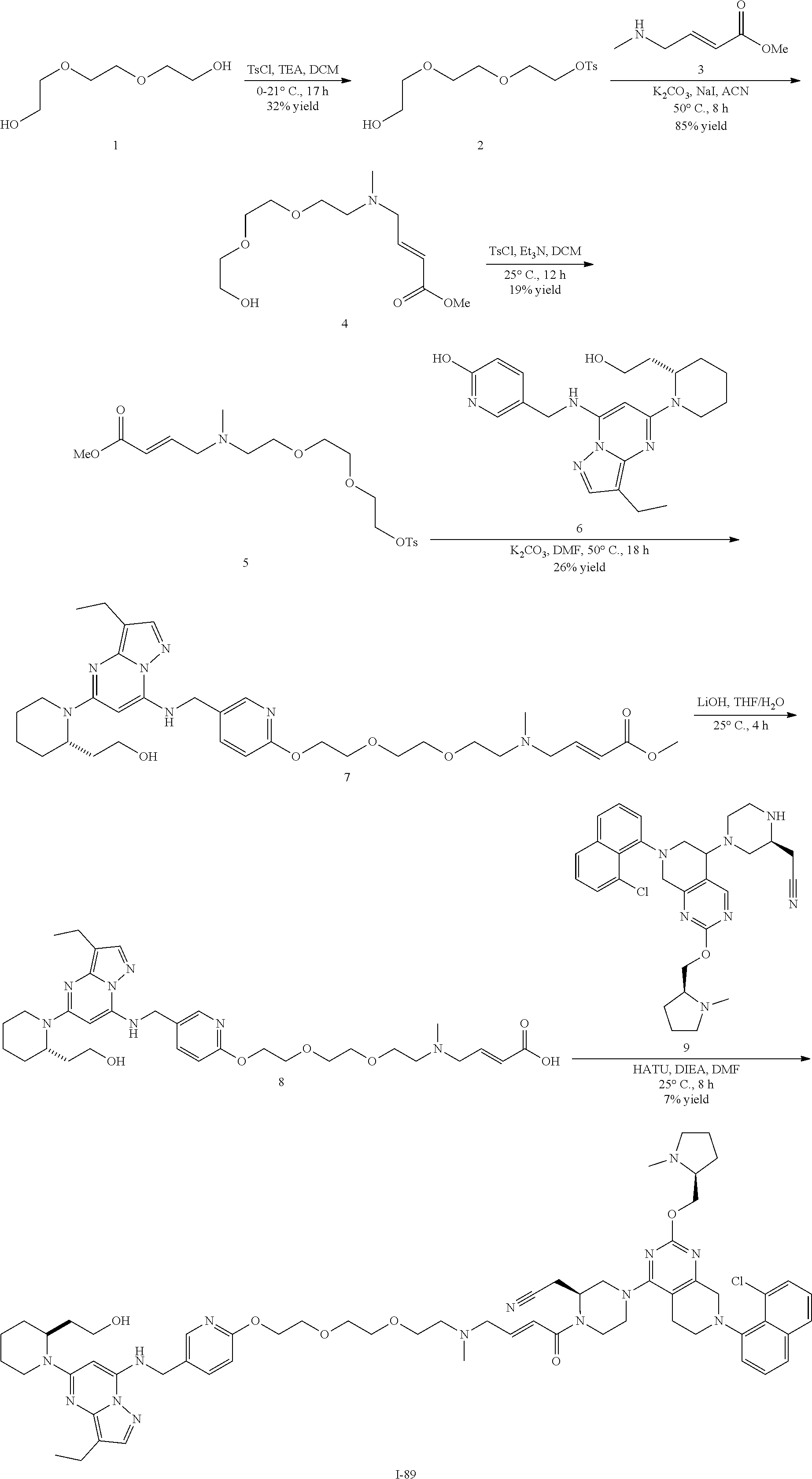

General Information: Synthetic route of compound 3 is reported in patent WO2017/100668. Synthetic route of compound 9 is reported in patent WO2021/108683.

Step 1. To a solution of 2-[2-(2-hydroxyethoxy)ethoxy]ethanol (20.0 g, 133 mmol, 18 mL, 1.0 equiv) and Et₃N (40.4 g, 400 mmol, 56 mL, 3.0 equiv) in DCM (200 mL) was stirred at 0° C. for 1 h, then the TosCl (25.4 g, 133 mmol, 1.0 equiv) in DCM (100 mL) was added to the mixture and stirred at 21° C. for 16 h. The reaction mixture was filtered and concentrated under reduced pressure to give a residue. The residue was purified by flash silica gel chromatography (ISCO®; 330 g SepaFlash® Silica Flash Column, Eluent of 5˜50% Ethylacetate/Petroleum ethergradient @ 100 mL/min) to afford 2-(2-(2-hydroxyethoxy)ethoxy)ethyl 4-methylbenzenesulfonate (13.0 g, 42.0 mmol, 32% yield,) as a light yellow liquid.

Step 2. To a solution of 2-(2-(2-hydroxyethoxy)ethoxy)ethyl 4-methylbenzenesulfonate (1.5 g, 4.9 mmol, 1.0 equiv) in ACN (20 mL) was added K₂CO₃ (2.1 g, 14.8 mmol, 3.0 equiv), NaI (738 mg, 4.9 mmol, 1.0 equiv), (E)-methyl 4-(methylamino)but-2-enoate (636 mg, 4.9 mmol, 1.0 equiv). The mixture was stirred at 50° C. for 8 h. The reaction mixture was partitioned between H₂O (100 mL) and EA (100 mL). The organic phase was separated, washed with water (50 mL×3), dried over Na₂SO₄, filtered and concentrated under reduced pressure to give a residue. The residue was purified by column chromatography (SiO₂, DCM: MeOH=20/1 to 10/1). Compound (E)-methyl 4-((2-(2-(2-hydroxyethoxy)ethoxy)ethyl)(methyl)amino)but-2-enoate (1.10 g, 4.21 mmol, 85% yield) was obtained as a light yellow oil. ¹H NMR: (400 MHz, CDCl3) δ=7.00-6.78 (t, J=15.6 Hz, 6.4 Hz, 1H), 6.00-5.82 (d, J=15.6 Hz, 1H), 3.67 (s, 3H), 3.66-3.49 (m, 1OH), 3.16 (d, J=6.4 Hz, 2H), 2.55 (t, J=5.6 Hz, 2H), 2.23 (s, 3H).

Step 3. To a solution of (E)-methyl 4-((2-(2-(2-hydroxyethoxy)ethoxy) ethyl)(methyl)amino)but-2-enoate (1.0 g, 3.8 mmol, 1.0 equiv) in DCM (10 mL) was added TsCl (540 mg, 7.6 mmol, 2.0 equiv) and TEA (1.2 g, 11.5 mmol, 1.6 mL, 3.0 equiv). The mixture was stirred at 25° C. for 12 h. The reaction mixture was concentrated under reduced pressure to remove solvent. The residue was diluted with water 50 mL and extracted with EA (50 mL×3). The combined organic layers were washed with water (50 mL×2), dried over Na₂SO₄ filtered and concentrated under reduced pressure to give a residue. The residue was purified by column chromatography (SiO₂, DCM/MeOH=20/1 to 10/1) to give (E)-methyl 4-(methyl(2-(2-(2-(tosyloxy)ethoxy)ethoxy)ethyl)amino)but-2-enoate (300 mg, 722 μmol, 19% yield) was obtained as a yellow oil. ¹H NMR: (400 MHz, CDCl₃) δ=7.76-7.70 (d, J=8.4 Hz, 2H), 7.30-7.25 (d, J=8.0 Hz, 2H), 6.89 (m, 1H), 5.94-5.90 (d, J=15.6 Hz, 1.2 Hz, 1H), 4.12-4.05 (m, 2H), 3.68-3.65 (m, 3H), 3.64-3.61 (m, 2H), 3.57-3.54 (m, 2H), 3.51 (d, J=1.7, 3.4 Hz, 2H), 3.49-3.47 (m, 2H), 3.15-3.11 (m, 2H), 2.55-2.48 (m, 2H), 2.36 (m, 3H), 2.23-2.16 (s, 3H).

Step 4. To a solution of (E)-methyl 4-(methyl(2-(2-(2-(tosyloxy)ethoxy)ethoxy)ethyl)amino)but-2-enoate (150 mg, 361 μmol, 1.5 equiv) in DMF (1.0 mL) was added K₂CO₃ (100 mg, 722 μmol, 3.0 equiv) and (S)-5-(((3-ethyl-5-(2-(2-hydroxyethyl)piperidin-1-yl)pyrazolo[1,5-a]pyrimidin-7-yl)amino)methyl)pyridin-2-ol (95 mg, 241 μmol, 1.0 equiv). The mixture was stirred at 50° C. for 18 h. The reaction mixture was diluted with H₂O (20 mL) and extracted with EA (20 mL×3). The combined organic layers were washed with brine (25 mL×2), dried over Na₂SO₄, filtered and concentrated under reduced pressure to give a residue. The crude product was purified by reversed-phase HPLC(column: Waters xbridge 150*25 mm 10 μm; mobile phase: [water(NH4HCO₃)-ACN]; B%: 40%-70%, 11 min) to give methyl (S,E)-methyl 4-((2-(2-(2-(5-(((3-ethyl-5-(2-(2-hydroxyethyl)piperidin-1-yl)pyrazolo [ 1,5-a]pyrimidin-7-yl)amino)methyl)pyridin-2-yl)ethoxy)ethoxy)ethyl)(methyl)amino)but-2-enoate (40 mg, 62 μmol, 26% yield) as a light yellow oil. ¹H NMR: (400 MHz, CDCl₃) δ=8.10-8.04 (d, J=2.4 Hz, 1H), 7.57-7.50 (m, 2H), 6.94-6.85 (m, 1H), 6.75-6.69 (d, J=8.4 Hz, 1H), 6.24-6.15 (m, 1H), 5.94-5.93 (m, 1H), 5.22 (s, 1H), 5.07-4.99 (m, 1H), 4.43-4.39 (m, 2H), 4.38-4.33 (m, 2H), 3.80-3.77 (m, 2H), 3.66-3.65 (m, 3H), 3.65-3.63 (m, 2H), 3.59-3.56 (m, 2H), 3.56-3.50 (m, 4H), 3.30-3.23 (m, 1H), 3.15-3.12 (m, 2H), 3.01-2.93 (m, 1H), 2.56-2.51 (m, 4H), 2.21 (s, 3H), 2.05-1.97 (m, 1H), 1.60 (s, 4H), 1.20-1.16 (m, 7H).

Step 5. To a solution of (S,E)-methyl 4-((2-(2-(2-(5-(((3-ethyl-5-(2-(2-hydroxyethyl)piperidin-1-yl)pyrazolo[ 1,5-a]pyrimidin-7-yl)amino)methyl)pyridin-2-yl)ethoxy)ethoxy)ethyl)(methyl)amino)but-2-enoate (30 mg, 46 μmol, 1.0 equiv) in THF (1 mL) and H₂O (0.3 mL), was added LiOH·H₂O (2.0 mg, 47 μmol, 1.0 equiv). The mixture was stirred at 25° C. for 4 h. The reaction mixture was quenched by addition H₂O (20 mL), added HCl (1 mol) to the mixture, ended the PH to 8, and extracted with EA (20 mL×3). The combined organic layers were washed with water (20 mL×3), dried over Na₂SO₄, filtered and concentrated under reduced pressure to give a residue. The residue was used for the next step without further purification. Compound (S,E)-4-((2-(2-(2-(5-(((3-ethyl-5-(2-(2-hydroxyethyl)piperidin-1-yl)pyrazolo[ 1,5-a]pyrimidin-7-yl)amino)methyl)pyridin-2-yl)ethoxy)ethoxy)ethyl)(methyl)amino)but-2-enoic acid (27 mg, 43 μmol, crude) was obtained as a yellow oil.

Step 5. To a solution of (S,E)-4-((2-(2-(2-(5-(((3-ethyl-5-(2-(2-hydroxyethyl)piperidin-1-yl)pyrazolo[ 1,5-a]pyrimidin-7-yl)amino)methyl)pyridin-2-yl)ethoxy)ethoxy)ethyl)(methyl) amino)but-2-enoic acid (27 mg, 43 μmol, 1.0 equiv) in DMF (1 mL) was added HATU (19 mg, 51 μmol, 1.2 equiv) and DIEA (16 mg, 129 μmol, 22 μL, 3.0 equiv), 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (28 mg, 43 μmol, 1.0 equiv). The mixture was stirred at 25° C. for 8 h. The reaction mixture was concentrated under reduced pressure to give a residue and was purified by the HPLC (column: Waters xbridge 150*25 mm*10 m; mobile phase: [water(NH₄HCO₃)-ACN]; B%: 54%-84%, 8 min) to give the 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((E)-4-((2-(2-(2-(5-(((3-ethyl-5-((S)-2-(2-hydroxyethyl)piperidin-1-yl)pyrazolo[ 1,5-a]pyrimidin-7-yl)amino)methyl)pyridin-2-yl)ethoxy)ethoxy)ethyl)(methyl)amino) but-2-enoyl)piperazin-2-yl)acetonitrile (4 mg, 3 μmol, 7% yield) as a yellow oil. LC-MS: MS (ES⁺): RT=2.494 min, m/z=570.5 [M/2+H⁺], LCMS method: 05. ¹H NMR: (400 MHz, MeOD) δ=8.22 (s, 1H), 7.87-7.82 (d, J=8.4 Hz, 1H), 7.81-7.76 (d, J=9.2 Hz, 1H), 7.73-7.66 (m, 2H), 7.57-7.49 (m, 2H), 7.42-7.32 (m, 2H), 7.13-6.92 (m, 1H), 6.86-6.75 (m, 2H), 5.58 (s, 1H), 5.13-5.00 (m, 1H), 4.78-4.70 (m, 4H), 4.61-4.54 (m, 3H), 4.48-4.44 (m, 2H), 4.40-4.29 (m, 2H), 4.24-4.18 (m, 1H), 4.09 (s, 4H), 3.88-3.83 (m, 4H), 3.77-3.69 (m, 6H), 3.63-3.58 (m, 2H), 3.23-3.17 (m, 2H), 3.12-2.99 (m, 6H), 2.93-2.89 (s, 3H), 2.77-2.51 (m, 4H), 2.44-2.35 (m, 1H), 2.23-1.93 (m, 6H), 1.79-1.49 (m, 9H), 1.32-1.29 (m, 2H), 1.27-1.22 (m, 3H), 0.98-0.85 (m, 1H).

### Example 74—Synthesis of Compound I-90

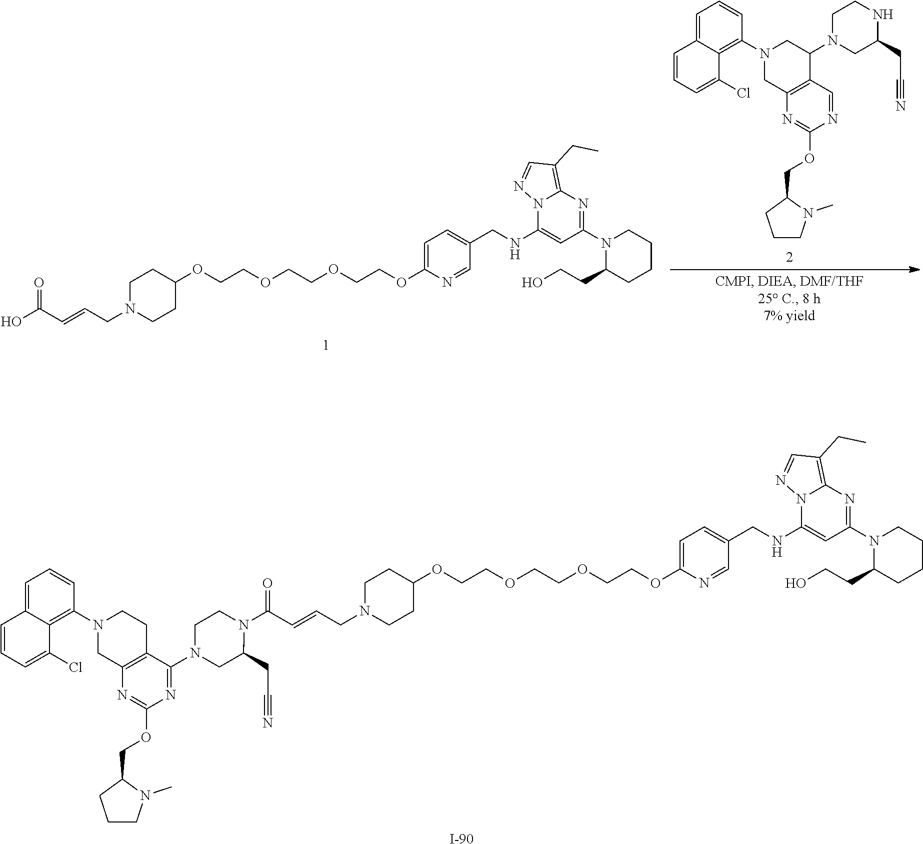

General Information: Synthetic route of compound 2 is provided in final report WO2021/108683.

To a solution of (S,E)-4-(4-(2-(2-(2-((5-(((3-ethyl-5-(2-(2-hydroxyethyl)piperidin-1-yl)pyrazolo[ 1,5-a]pyrimidin-7-yl)amino)methyl)pyridin-2-yl)oxy)ethoxy)ethoxy)ethoxy) piperidin-1-yl)but-2-enoic acid (30 mg, 57 μmol, 1.0 equiv) in THF (2 mL) and DMF (1 mL), was added CMPI (22 mg, 86 μmol, 1.5 equiv) and DIEA (22 mg, 172 μmol, 30 μL, 3.0 equiv), 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (40 mg, 57 μmol, 1.0 equiv). The mixture was stirred at 25° C. for 8 h to give a brown solution. The reaction mixture was partitioned between ethyl acetate (50 mL) and water (50 mL). The organic phase was separated, washed with water (30 mL×3), dried over Na₂SO₄, filtered and concentrated under reduced pressure to give a residue. The crude product was purified by reversed-phase HPLC (column: Phenomenex luna C18 150*25 mm*10 m; mobile phase: [water(FA)-ACN]; B%: 14%-44%, 10.5 min) to give the 2-((S)-4-(7-(8-chloronaphthalen-1-yl)-2-(((S)-1-methylpyrrolidin-2-yl)methoxy)-5,6,7,8-tetrahydropyrido [3,4-d]pyrimidin-4-yl)-1-((E)-4-(4-(2-(2-(2-((5-(((3-ethyl-5-((S)-2-(2-hydroxyethyl)piperidin-1-yl)pyrazolo[ 1,5-a]pyrimidin-7-yl)amino)methyl)pyridin-2-yl)oxy)ethoxy)ethoxy)ethoxy)piperidin-1-yl)but-2-enoyl)piperazin-2-yl)acetonitrile (5 mg, 4 μmol, 7% yield) as a yellow oil. LC-MS: MS (ES⁺): RT=2.710 min, m/z=605.6 [M/2+H⁺], LCMS method05. ¹H NMR: (400 MHz, CD₃OD) 6=8.57-8.42 (m, 1H), 8.21-8.20 (d, J=1.6 Hz, 1H), 7.87-7.82 (m, 1H), 7.81-7.75 (m, 1H), 7.72-7.68 (m, 1H), 7.67-7.64 (m, 1H), 7.56-7.48 (m, 2H), 7.41-7.30 (m, 2H), 6.87-6.78 (m, 2H), 5.57-5.52 (m, 1H), 5.12-5.07 (m, 1H), 4.85-4.78 (m, 4H), 4.73-4.66 (m, 2H), 4.59-4.46 (m, 4H), 4.45-4.41 (m, 2H), 4.39-4.31 (m, 1H), 4.26-4.18 (m, 1H), 4.14-4.01 (m, 2H), 3.86-3.82 (m, 2H), 3.79-3.72 (m, 2H), 3.70-3.68 (m, 2H), 3.71-3.63 (m, 3H), 3.63-3.58 (m, 5H), 3.56-3.47 (m, 3H), 3.47-3.44 (m, 1H), 3.38-3.37 (d, J=3.6 Hz, 2H), 3.29-3.24 (m, 1H), 3.22-3.14 (m, 2H), 3.13-3.03 (m, 2H), 3.02 (m, 6H), 2.78-2.69 (m, 1H), 2.69-2.64 (m, 1H), 2.60-2.54 (m, 2H), 2.31 (s, 1H), 2.14-2.06 (m, 2H), 1.99-1.91 (m, 2H), 1.82-1.61 (m, 8H), 1.57-1.46 (m, 1H), 1.29-1.17 (m, 3H).

### Example 75—Synthesis of Compound I-91

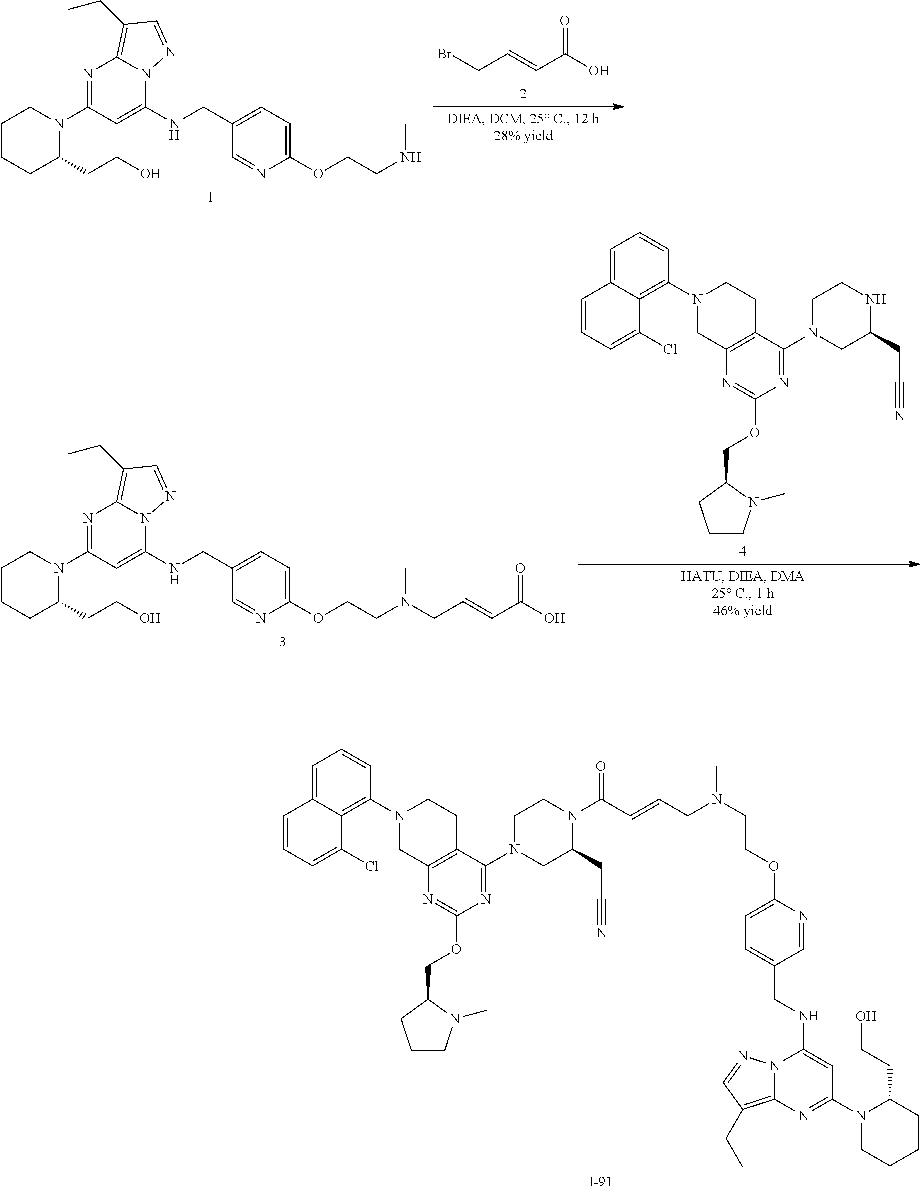

General Information: Synthetic route of compound 4 is reported in WO2021/108683.

Step 1. To a solution of 2-[(2S)-1-[3-ethyl-7-[[6-[2-(methylamino)ethoxy]-3-pyridyl]methylamino]pyrazolo[1,5-a]pyrimidin-5-yl]-2-piperidyl]ethanol (1.0 g, 1.7 mmol, 1.0 equiv, TFA) in THF (10 mL) was added (E)-4-bromobut-2-enoic acid (233 mg, 1.4 mmol, 33 μL, 0.8 equiv) and DIEA (683 mg, 5.3 mmol, 921 μL, 3 equiv). The mixture was stirred at 25° C. for 12 h. The reaction mixture was concentrated under reduced pressure to remove solvent. The residue was purified by the prep-HPLC (column: Phenomenex C18 250*50 mm*10%μm; mobile phase: [water (NH₄HCO₃)-ACN]; B%: 17%-47%, 8 min) to give the (E)-4-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethyl-methyl-amino]but-2-enoic acid (270 mg, 497 μmol, 28% yield) as a white solid. ¹H NMR: (400 MHz, CDCl₃) δ=8.13 (d, J=2.0 Hz, 1H), 7.55-7.72 (m, 2H), 6.91 (d, J=8.4 Hz, 2H), 6.31-6.53 (m, 1H), 5.72 (d, J=16.0 Hz, 1H), 5.07-5.24 (m, 1H), 4.41-4.64 (m, 2H), 4.32 (d, J=4.0 Hz, 2H), 3.52-3.92 (m, 2H), 3.25-3.48 (m, 1H), 2.95-3.18 (m, 3H), 2.67-2.83 (m, 2H), 2.56-2.64 (m, 2H), 2.45 (s, 3H), 2.09-2.20 (m, 1H), 1.64-1.85 (m, 9H), 1.15-1.30 (m, 3H).

Step 2. To a mixture of (E)-4-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethyl-methyl-amino]but-2-enoic acid (40 mg, 74 μmol, 1.0 equiv) and 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]piperazin-2-yl]acetonitrile (40 mg, 61 μmol, 1.1 equiv, TFA) and DIEA (29 mg, 223 μmol, 39 L, 3.0 equiv) in DMA (2 mL) was added HATU (34 mg, 89 μmol, 1.2 equiv) in one portion at 25° C. for 1 h under N₂. The reaction mixture was concentrated under reduced pressure to remove solvent. The residue was purified by the prep-HPLC(column: Phenomenex C18 150*25 mm*10%μm; mobile phase: [water(NH4HCO₃)-ACN]; B%: 60%-90%, 5 min) to give the 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-[(E)-4-[2-[[5-[[[3-ethyl-5-[(2S)-2-(2-hydroxyethyl)-1-piperidyl]pyrazolo [1,5-a]pyrimidin-7-yl]amino]methyl]-2-pyridyl]oxy]ethyl-methyl-amino]but-2-enoyl]piperazin-2-yl]acetonitrile (37 mg, 34 μmol, 46% yield, 98% purity) as a white solid. LC-MS: MS (ES⁺): RT=2.301 min, m/z=1051.7 [M+H⁺]. LCMS Method: 05. ¹H NMR: (400 MHz, MeOH) δ=8.21 (s, 1H), 7.74-7.84 (m, 2H), 7.64-7.71 (m, 1H), 7.61 (s, 1H), 7.45-7.55 (m, 2H), 7.27-7.39 (m, 2H), 6.61-6.94 (m, 3H), 5.51 (s, 1H), 4.73-4.82 (m, 2H), 4.50-4.66 (m, 4H), 4.41-4.47 (m, 2H), 4.22-4.40 (m, 4H), 3.92-4.22 (m, 4H), 3.63-3.77 (m, 2H), 3.43-3.61 (m, 4H), 3.19 (s, 2H), 3.05-3.12 (m, 2H), 2.99 (d, J=12.0 Hz, 1H), 2.81-2.91 (m, 3H), 2.62-2.77 (m, 2H), 2.48-2.59 (m, 5H), 2.30-2.43 (m, 4H), 1.98-2.14 (m, 2H), 1.78-1.85 (m, 2H), 1.60-1.72 (m, 6H), 1.26-1.39 (m, 2H), 1.18-1.25 (m, 3H).

### Example 76—Synthesis of Compound I-92

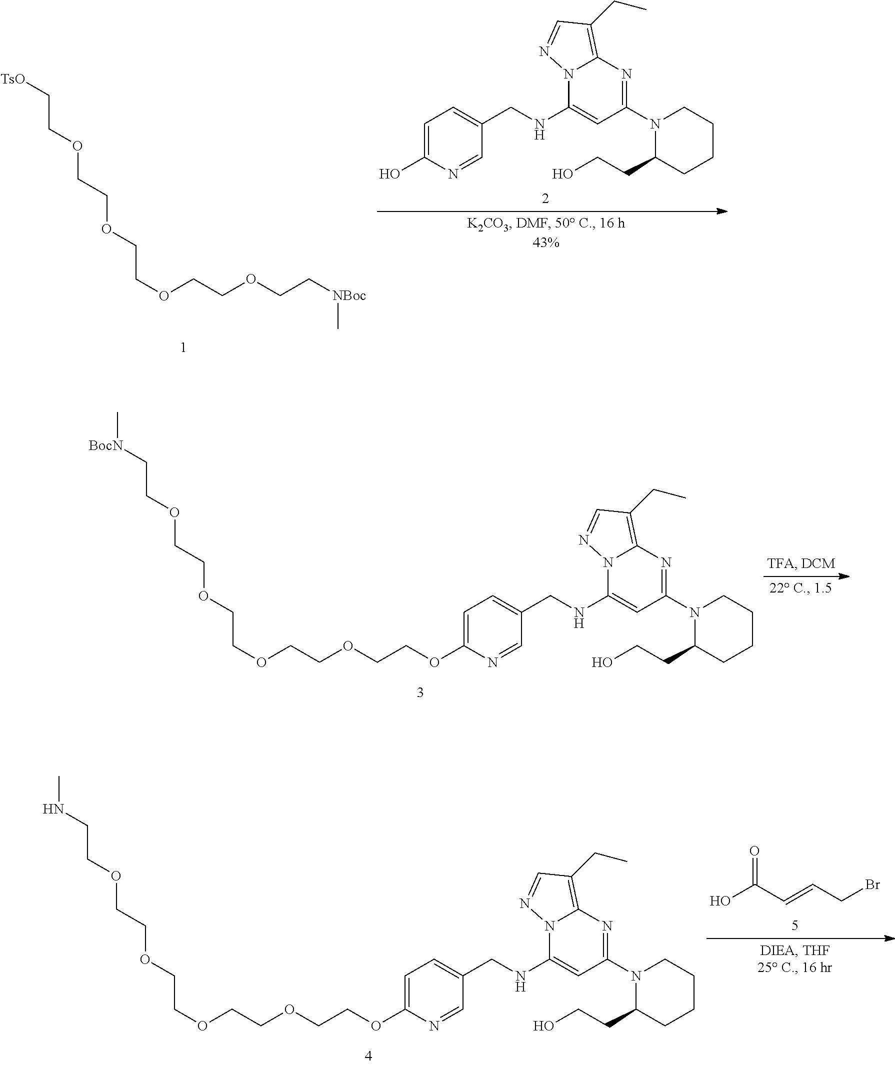 [SMILES: Cc1ccc(S(=O)(=O)OCCOCCOCCOCCOCCN(C)C(=O)OC(C)(C)C)cc1 ; CCc1cnn2c(NCc3ccc(O)nc3)cc(N3CCCC[C@H]3CCO)nc12 ; CCc1cnn2c(NCc3ccc(OCCOCCOCCOCCOCCN(C)C(=O)OC(C)(C)C)nc3)cc(N3CCCC[C@H]3CCO)nc12 ; CCc1cnn2c(NCc3ccc(OCCOCCOCCOCCOCCNC)nc3)cc(N3CCCC[C@H]3CCO)nc12 ; O=C(O)/C=C/CBr]

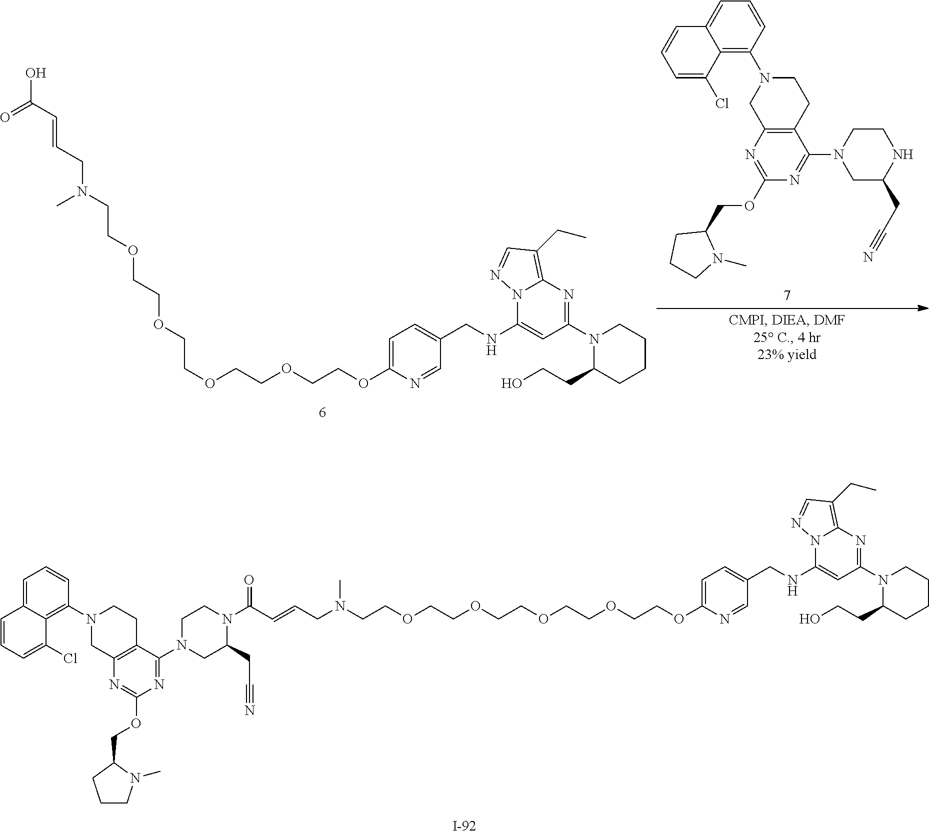

General Information: Synthetic route of compound 1 is reported in WO2021/178339. Synthetic route of compound 7 is reported in WO2021/108683.

Step 1. A solution of 2-[2-[2-[2-[2-[tert-butoxycarbonyl(methyl) amino]ethoxy]ethoxy]ethoxy]ethoxy]ethyl 4-methylbenzenesulfonate (383 mg, 757 μmol, 1.5 equiv), 5-[[[3-ethyl-5-[(2S)-2-(2-hydroxyethyl)-1-piperidyl]pyrazolo[1,5-a]pyrimidin-7-yl]amino]methyl]pyridin-2-ol (200 mg, 504 μmol, 1.0 equiv) and K₂CO₃ (139 mg, 1.01 mmol, 2.0 equiv) in DMF (15 mL) was stirred at 50° C. for 16 h. The residue was quenched with H₂O (30 mL) and extracted with EtOAc (30 mL×3). The combined organic layers were washed with brine (80 mL), dried over Na₂SO₄, filtered and concentrated under reduced pressure to give a residue. The residue was purified by prep-HPLC (column: Phenomenex C18 150×25 mm×10 μm; mobile phase: [water(NH₄HCO₃)-ACN]; B%: 45%-75%, 8 min) to give tert-butyl N-[2-[2-[2-[2-[2-[[5-[[[3-ethyl-5-[(2S)-2-(2-hydroxyethyl)-1-piperidyl]pyrazolo[ 1,5-a]pyrimidin-7-yl]amino]methyl]-2-pyridyl]oxy]ethoxy]ethoxy]ethoxy]ethoxy]ethyl]-N-methyl-carbamate (160 mg, 219 μmol, 43% yield) as brown oil. ¹H NMR: (400 MHz, CD₃C₁) δ =8.15 (d, J=2.0 Hz, 1H), 7.64 (s, 1H), 7.61 (d, J=2.4, 8.2 Hz, 1H), 6.99 (s, 1H), 6.81 (d, J=8.4 Hz, 1H), 6.29 (t, J=5.6 Hz, 1H), 5.29 (s, 1H), 5.16-5.07 (m, 1H), 4.50-4.43 (m, 4H), 3.89-3.83 (m, 2H), 3.75-3.54 (m, 18H), 3.42-3.30 (m, 3H), 3.11-2.99 (m, 1H), 2.91 (s, 3H), 2.60 (d, J=2.8, 7.2 Hz, 2H), 2.14-2.03 (m, 1H), 1.71 (d, J=2.8, 6.8 Hz, 2H), 1.45 (s, 9H), 1.28-1.23 (m, 6H).

Step 2. TFA (0.5 mL, 6.8 mmol, 31.0 equiv) was added to a solution of tert-butyl N-[221[2-[2-[2-2[[5-5[[[3-ethyl-5-1[(2S)-2-(2-hydroxyethyl)-1-piperidyl]pyrazolo[1,5-a]pyrimidin-7-yl]amino]methyl]-2 pyridyl]oxy]ethoxy]ethoxy]ethoxy]ethoxy]ethyl]-N-methyl-carbamate (160 mg, 219 μmol, 1.0 equiv) in DCM (3 mL) at 22° C. The mixture was stirred at 22° C. for 1.5 h. The reaction mixture was concentrated under reduced pressure to give 2-[(2S)-1-[3-ethyl-7-[[6-[2[2-[2-[2-[2-(methylamino)ethoxy]ethoxy]ethoxy]ethoxy]ethoxy]-3-pyridyl]methylamino]pyrazolo[1,5-a]pyrimidin-5-yl]-2-piperidyl]ethanol (170 mg, 159 μmol, TFA) as light yellow oil.

Step 3. To a solution of (S)-2-(1-(7-(((6-(5,8,11,14-tetraoxa-2-azahexadecan-16-yloxy)pyridin-3-yl)methyl)amino)-3-ethylpyrazolo[1,5-a]pyrimidin-5-yl)piperidin-2-yl)ethanol (130 mg, 206 μmol, 1.0 equiv) in THF (1 mL), was added DIEA (80 mg, 619 μmol, 108 μL, 3.0 equiv), (E)-4-bromobut-2-enoic acid (34 mg, 206 μmol, 1.0 equiv). The mixture was stirred at 25° C. for 16 h. The reaction mixture was concentrated under reduced pressure to give a residue. The crude product was purified by reversed-phase HPLC (column: Phenomenex C18 150*25 mm*10 μm; mobile phase: [water(NH₄HCO₃)-ACN]; B%: 22%-52%, 5 min) to give the (S,E)-1-((5-(((3-ethyl-5-(2-(2-hydroxyethyl)piperidin-1-yl)pyrazolo[1,5-a]pyrimidin-7-yl)amino)methyl)pyridin-2-yl)oxy)-15-methyl-3,6,9,12-tetraoxa-15-azanonadec-17-en-19-oic acid (26 mg, 36 μmol) as a yellow oil. ¹H NMR: (400 MHz, CD₃C₁) δ =8.21-8.15 (d, J=2.4 Hz, 1H), 7.68-7.61 (m, 2H), 7.01-6.91 (m, 1H), 6.81-6.79 (d, J=8.4 Hz 1H), 6.02-5.98 (d, J=16 Hz, 1H), 5.33 (s, 1H), 5.12-5.10 (d, J=11.2 Hz 1H), 4.51-4.45 (m, 4H), 3.89-3.88 (d, J=4.4 Hz, 2H), 3.74-3.72 (m, 2H), 3.70-3.66 (m, 8H), 3.65-3.62 (m, 4H), 3.61 (s, 1H), 3.39-3.32 (m, 1H), 3.26 (d, J=1.2, 6.2 Hz, 2H), 3.10-3.02 (m, 1H), 2.60 (s, 4H), 2.36-2.34 (s, 3H), 2.27-2.22 (m, 2H), 2.11-2.08 (m, 2H), 1.75-1.71 (m, 2H), 1.29-1.26 (m, 6H).

Step 4. To a solution of (S,E)-1-((5-(((3-ethyl-5-(2-(2-hydroxyethyl)piperidin-1-yl)pyrazolo[1,5-a]pyrimidin-7-yl)amino)methyl)pyridin-2-yl)oxy)-15-methyl-3,6,9,12-tetraoxa-15-azanonadec-17-en-19-oic acid (20 mg, 28 μmol, 1.0 equiv) in DMF (1.0 mL) was added CMPI (10 mg, 42. μmol, 1.5 equiv) and DIEA (11 mg, 84 μmol, 14 μL, 3.0 equiv), 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (15 mg, 28 μmol, 1.0 equiv). The mixture was stirred at 25° C. for 4 h to give a brown solution. The crude product was purified by reversed-phase HPLC (column: Phenomenex C18 150*25 mm*10 m; mobile phase: [water(FA)-ACN]; B%: 10%-40%, 5 min) to give the 2-((S)-4-(7-(8-chloronaphthalen-1-yl)-2-(((S)-1-methylpyrrolidin-2-yl)methoxy)-5,6,7,8-tetrahydropyrido [3,4-d]pyrimidin-4-yl)-1-((E)-1-((5-(((3-ethyl-5-((S)-2-(2-hydroxyethyl)piperidin-1-yl)pyrazolo[1,5-a]pyrimidin-7-yl)amino)methyl)pyridin-2-yl)oxy)-15-methyl-3,6,9,12-tetraoxa-15-azanonadec-17-en-19-oyl)piperazin-2-yl)acetonitrile (8 mg, 6 μmol, 23% yield) as a white solid. LC-MS: MS (ES⁺): RT=2.344 min, m/z=614.6 [M/2+H⁺]. LCMS Method: 05. ¹H NMR: (400 MHz, CD₃OD) 6=8.57-8.53 (m, 1H), 8.22-8.17 (d, J=2.0 Hz, 1H), 7.85-7.80 (d, J=8.4 Hz 1H), 7.78-7.73 (d, J=3.6 Hz 1H), 7.70-7.64 (m, 2H), 7.54-7.46 (m, 2H), 7.40-7.29 (m, 2H), 6.89-6.69 (m, 3H), 5.54 (s, 1H), 5.13-5.02 (m, 1H), 4.85-4.76 (m, 2H), 4.57-4.53 (m, 2H), 4.45-4.28 (m, 6H), 4.22-4.17 (m, 1H), 4.07-4.01 (m, 1H), 3.84-3.80 (m, 2H), 3.76-3.70 (m, 1H), 3.67-3.54 (m, 18H), 3.42-3.38 (m, 1H), 3.29-3.24 (m, 2H), 3.20-3.14 (m, 2H), 3.09-3.01 (m, 2H), 2.99-2.90 (m, 2H), 2.60-2.53 (m, 1H), 2.56 (d, J=7.6 Hz, 9H), 2.40-2.34 (m, 3H), 2.19-2.06 (m, 2H), 1.95-1.88 (m, 2H), 1.76-1.62 (m, 7H), 1.55-1.46 (m, 1H), 1.26-1.21 (m, 3H).

### Example 77—Synthesis of Compound I-93

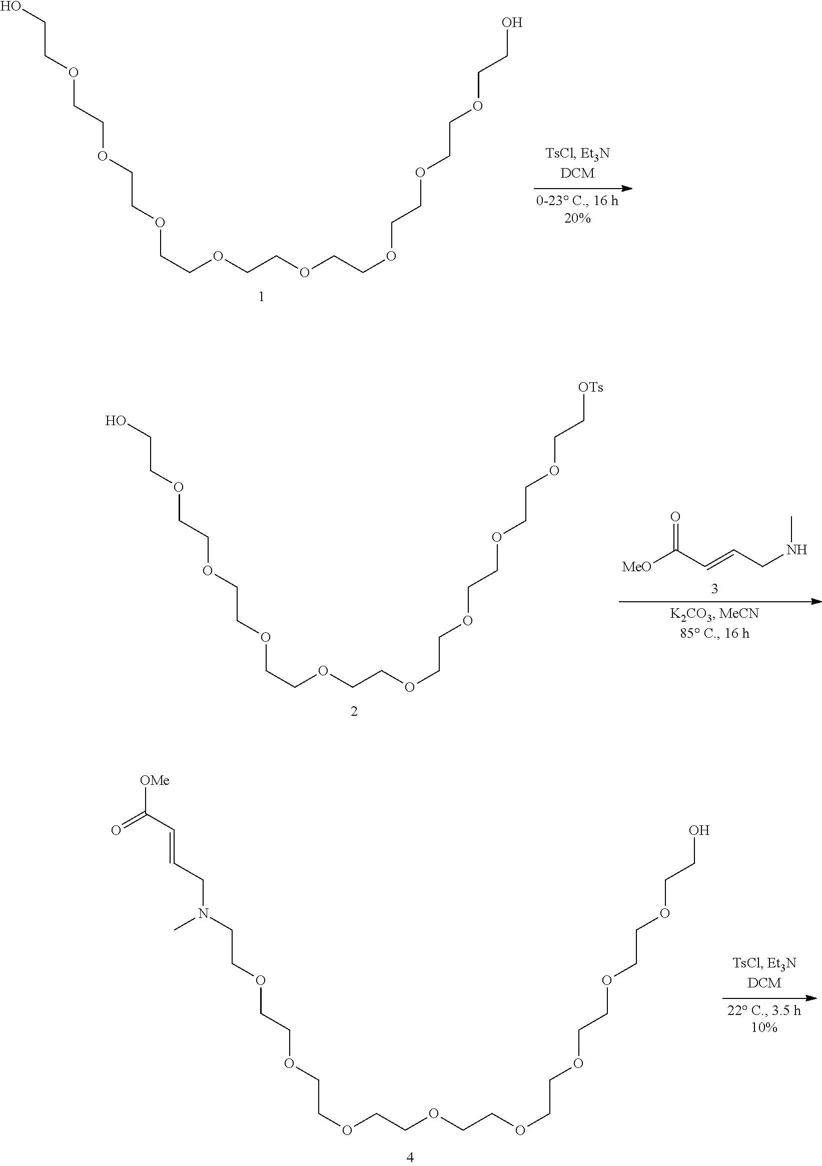

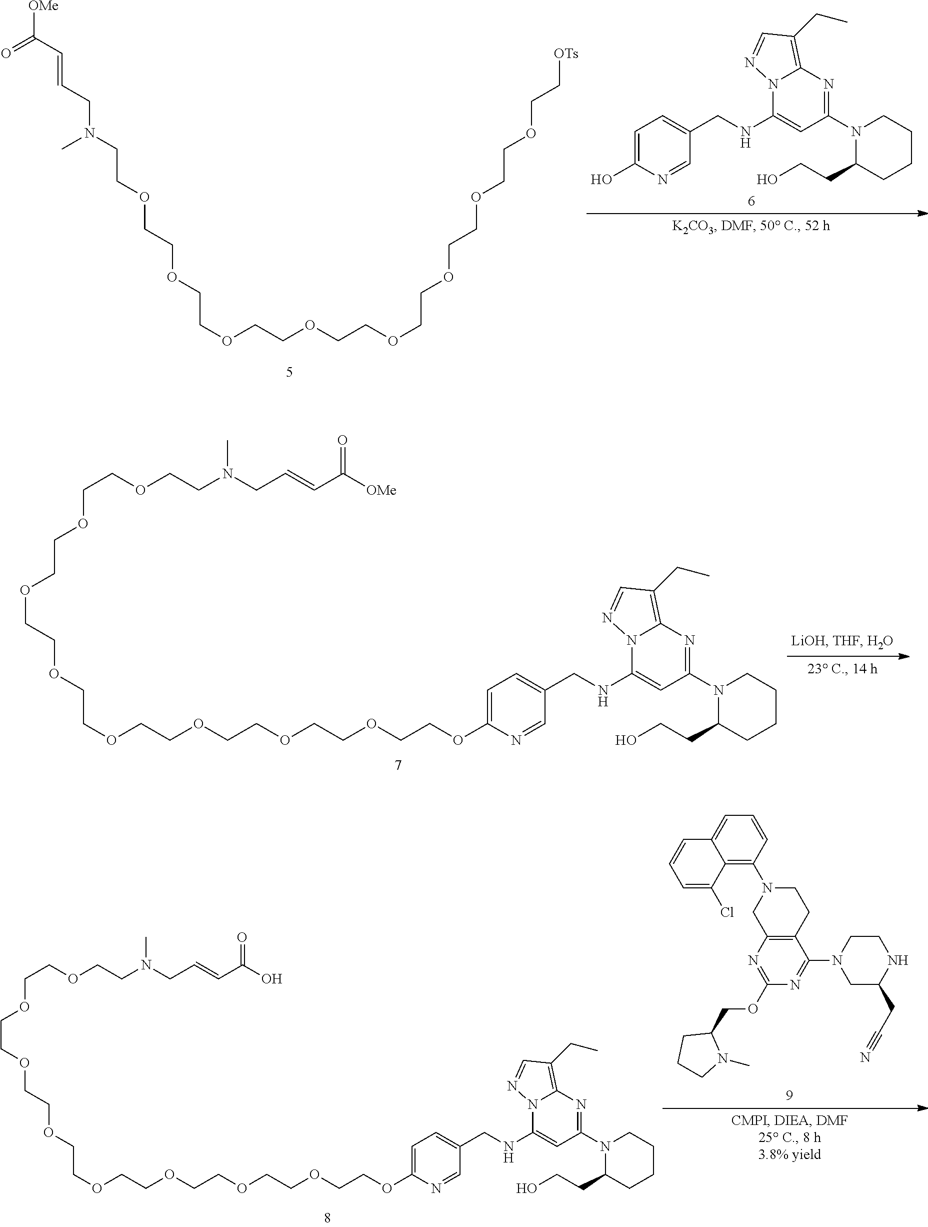 [SMILES: COC(=O)/C=C/CN(C)CCOCCOCCOCCOCCOCCOCCOCCOCCOS(=O)(=O)c1ccc(C)cc1 ; CCc1cnn2c(NCc3ccc(O)nc3)cc(N3CCCC[C@H]3CCO)nc12 ; CCc1cnn2c(NCc3ccc(OCCOCCOCCOCCOCCOCCOCCOCCOCCN(C)C/C=C/C(=O)OC)nc3)cc(N3CCCC[C@H]3CCO)nc12 ; CCc1cnn2c(NCc3ccc(OCCOCCOCCOCCOCCOCCOCCOCCOCCN(C)C/C=C/C(=O)O)nc3)cc(N3CCCC[C@H]3CCO)nc12 ; CN1CCC[C@H]1COc1nc2c(c(N3CCN[C@@H](CC#N)C3)n1)CCN(c1cccc3cccc(Cl)c13)C2]

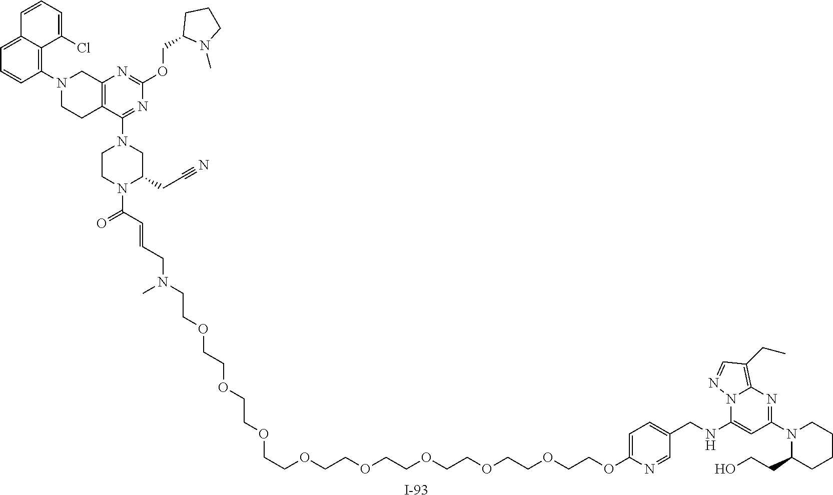

General Information: Synthetic route of compound 9 is reported in WO2021/108683, 2021, A1.

Step 1. To a solution of 2-[2-[2-[2-[2-[2-[2-[2-(2-hydroxyethoxy)ethoxy]ethoxy]ethoxy]ethoxy]ethoxy]ethoxy]ethoxy]ethanol (5.0 g, 12.0 mmol, 1.0 equiv) in DCM (50 mL) was added TsCl (935 mg, 13.2 mmol, 1.1 equiv) and Et₃N (3.7 g, 36.1 mmol, 3.0 equiv) in DCM(20 ml) at 0° C., then the mixture was stirred at 22° C. for 16 h. The reaction mixture was diluted with H₂O (100 mL) and extracted with DCM (100 mL×3). The combined organic layers were washed with brine (30 mL×3), dried over Na₂SO₄, filtered and concentrated under reduced pressure to give a residue. The residue was purified by prep-HPLC (column: YMC Triart C18 250×50 mm×7 m; mobile phase: [water(NH₄HCO₃)-ACN]; B%: 25%-55%, 20 min) to give 2-[22[2-[2-[[2-[2-[2-(2-hydroxyethoxy)ethoxy]ethoxy]ethoxy]ethoxy]ethoxy]ethoxy]ethoxy]ethyl 4-methylbenzenesulfonate (1.5 g, 7.2 mmol, 20% yield) as a colorless oil. ¹H NMR: ¹H NMR (400 MHz, CDCl₃) δ=7.81 (d, J=8.0 Hz, 2H), 7.35 (d, J=8.0 Hz, 2H), 4.17 (t, J=4.8 Hz, 2H), 3.73-3.60 (m, 34H), 2.45 (s, 3H).

Step 2. To a solution of 2-[2-[2-[2-[2-[2-[2-[2-(2-hydroxyethoxy)ethoxy]ethoxy]ethoxy]ethoxy]ethoxy]ethoxy]ethoxy]ethyl 4-methyl benzenesulfonate (3.5 g, 6.2 mmol, 1.0 equiv) in MeCN (50 mL) was added K₂CO₃ (2.6 g, 18.5 mmol, 3.0 equiv) and methyl (E)-4-(methylamino)but-2-enoate (1.5 g, 6.2 mmol, 1.0 equiv, TFA salt). The mixture was stirred at 85° C. for 16 h. The reaction mixture was concentrated under reduced pressure to remove solvent. The residue was purified by flash silica gel chromatography (ISCO@; 20 g SepaFlash@ Silica Flash Column, Eluent of 0-10% MeOH/DCM gradient @ 20 mL/min) to give methyl (E)-4-[2-[2[2-[2-[[2-[2-[2-(2-hydroxyethoxy)ethoxy]ethoxy]ethoxy]ethoxy]ethoxy]ethoxy]ethoxy]ethyl-methyl-amino]but-2-enoate (2.1 g, crude) as brown gum.

Step 3. To a solution of methyl (E)-4-[2-[2-[2-[2-[2-[2-[2-[2-(2-hydroxyethoxy)ethoxy]ethoxy]ethoxy]ethoxy]ethoxy]ethoxy]ethoxy]ethyl-methyl-amino]but-2-enoate (2.1 g, 4.1 mmol, 1.0 equiv) and Et₃N (2.5 g, 24.4 mmol, 3.4 mL, 6.0 equiv) in DCM (30 mL) was added TosCl (2.3 g, 12.2 mmol, 3.0 equiv) in DCM (20 mL). Then the mixture was stirred at 22° C. for 3.5 h. The reaction mixture was concentrated under reduced pressure to remove solvent. The residue was purified by prep-HPLC(column: Phenomenex luna C18 250×50 mm×10 m; mobile phase: [water(NH4HCO₃)-ACN]; B%: 15%-45%, 30 min) to give methyl (E)-4-1[methyl-1[[21[2-1[2-1[2-1[2-1[2-1[2-1[2-(p-tolylsulfonyloxy)ethoxy]ethoxy]ethoxy]ethoxy]ethoxy]ethoxy]ethoxy]ethoxy]ethyl]amino]but-2-enoate (396 mg, 402 μmol, 10% yield) obtained as brown oil. ¹H NMR: ¹H NMR (400 MHz, CD₃C₁) δ =7.81 (d, J=8.0 Hz, 2H), 7.35 (d, J=8.0 Hz, 2H), 7.04-6.90 (m, 1H), 6.15-6.03 (m, 1H), 4.21-4.10 (m, 2H), 3.76 (s, 2H), 3.74-3.68 (m, 5H), 3.67-3.61 (m, 28H), 3.59 (s, 5H), 2.51 (s, 2H), 2.46 (s, 3H).

Step 4. To a solution of methyl (E)-4-[methyl-[2-[2-[2-[2-[2-[2-[2-[2-[2-(p-tolylsulfonyloxy)ethoxy]ethoxy]ethoxy]ethoxy]ethoxy]ethoxy]ethoxy]ethoxy]ethyl]amino]but-2-enoate (252 mg, 370 μmol, 1.5 equiv) in DMF (2 mL) was added K₂CO₃ (102 mg, 741 μmol, 3.0 equiv) and 5-[[[3-ethyl-5-[(2S)-2-(2-hydroxyethyl)-1-piperidyl]pyrazolo[1,5-a]pyrimidin-7-yl]amino]methyl]pyridin-2-ol (100 mg, 247 μmol, 98% purity, 1.0 equiv). The mixture was stirred at 50° C. for 25 h. The mixture was filtered and concentrated to give a residue. The residue was purified by prep-HPLC (column: Phenomenex C18 150×25 mm×10 μm; mobile phase: [water(NH₄HCO₃)-ACN]; B%: 32%-62%, 5 min) to give methyl (E)-4-[2-[2-[2-[2-[2-[2-[2-[2-[2-[[5-1[[[3-ethyl-5-1[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethoxy]ethoxy]ethyl-methyl-amino]but-2-enoate (68 mg, 47 μmol, 19% yield) as a brown oil. ¹H NMR: ¹H NMR (400 MHz, CDCl₃) δ=8.16-8.13 (m, 1H), 7.64-7.63 (m, 1H), 7.62-7.59 (m, 1H), 7.44-7.30 (m, 1H), 6.98 (t, J=6.4, 15.6 Hz, 1H), 6.80 (d, J=8.8 Hz, 1H), 6.36-6.29 (m, 1H), 6.02 (d, J=15.6 Hz, 1H), 5.37-5.25 (m, 2H), 5.16-5.01 (m, 2H), 4.52-4.42 (m, 6H), 3.74 (s, 3H), 3.67-3.62 (m, 34H), 3.40-3.32 (m, 2H), 3.32-3.27 (m, 2H), 3.17-3.08 (m, 2H), 3.06 (d, J=15.2 Hz, 2H), 2.70 (t, J=4.8 Hz, 3H), 2.49-2.44 (m, 1H), 2.36 (s, 3H), 1.25 (s, 3H).

Step 5. To a solution of methyl (E)-4-[2-[2-[2-[2-[2-[2-[2-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethoxy]ethoxy]ethyl-methyl-amino]but-2-enoate (68 mg, 75 μmol, 1.0 equiv) in H₂O (0.3 mL) and THF (1 mL)was added LiOH·H₂O (6 mg, 150 μmol, 2.0 equiv). The mixture was stirred at 23° C. for 14 h. The reaction mixture was filtered and concentrated to give a residue. The crude compound was used to next step directly without purification to give (E)-4-[2-[2-[2-[2-[2-[2-[2-[2-[2-[[5-[[[3-ethyl-5-[(2S)-2-(2-hydroxyethyl)-1-piperidyl]pyrazolo1[1,5-a]pyrimidin-7-yl]amino]methyl]-2-pyridyl]oxy]ethoxy]ethoxy]ethoxy]ethoxy]ethoxy]ethoxy]ethoxy]ethoxy]ethyl-methyl-amino]but-2-enoic acid (66.0 mg, crude) as a yellow solid.

Step 6. To a solution of (S,E)-1-((5-(((3-ethyl-5-(2-(2-hydroxyethyl)piperidin-1-yl)pyrazolo[1,5-a]pyrimidin-7-yl)amino)methyl)pyridin-2-yl)oxy)-27-methyl-3,6,9,12,15,18,21,24-octaoxa-27-azahentriacont-29-en-31-oic acid (50.0 mg, 56 μmol, 1.0 equiv) in DMF (1 mL) was added CMPI (21 mg, 84 μmol, 1.5 equiv) and DIEA (21 mg, 169 μmol, 29 L, 3.0 equiv), 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (30 mg, 56 μmol, 1.0 equiv. The mixture was stirred at 25° C. for 8 h to give a brown solution. The mixture was filtered and was purified by the (HPLC purification. column: Phenomenex C18 150*25 mm*10 μm; mobile phase: [water(FA)-ACN]; B%: 12%-42%, 5 min) to give the 2-((S)-4-(7-(8-chloronaphthalen-1-yl)-2-(((S)-1-methylpyrrolidin-2-yl)methoxy)-5,6,7,8-tetrahydropyrido[3,4d]pyrimidin4-yl)-1-((E)-1-((5-(((3-ethyl-5-((S)-2-(2-hydroxyethyl)piperidin-1-yl)pyrazolo[1,5-a]pyrimidin-7-yl)amino)methyl)pyridin-2-yl)oxy)-27-methyl-3,6,9,12,15,18,21,24-octaoxa-27-azahentriacont-29-en-31-oyl)piperazin-2-yl)acetonitrile (4 mg, 2 μmol, 3% yield) as a white solid. LC-MS: MS (ES⁺): RT=2.370 min, m/z=702.7 [M/2+H⁺], LCMS method: 05. ¹H NMR: (400 MHz, CD₃OD) 6=8.54-8.49 (m, 1H), 8.24-8.19 (d, J=1.6 Hz, 1H), 7.87-7.82 (d, J=8.0 Hz, 1H), 7.80-7.75 (d, J=8.4 Hz, 1H), 7.72-7.63 (m, 2H), 7.56-7.48 (m, 2H), 7.31 (m, 2H), 6.91-6.79 (m, 3H), 5.55 (s, 1H), 4.84-4.78 (m, 3H), 4.68-4.60 (m, 2H), 4.56-4.54 (m, 2H), 4.53-4.44 (m, 2H), 4.44-4.40 (m, 2H), 4.38-4.32 (m, 1H), 4.25-4.19 (m, 1H), 4.15-4.10 (m, 1H), 4.08-4.01 (m, 1H), 3.86-3.82 (m, 2H), 3.71-3.55 (m, 38H), 3.05-2.96 (m, 3H), 2.95-2.81 (m, 7H), 2.61-2.43 (m, 6H), 2.35-2.26 (m, 1H), 2.13-2.02 (m, 3H), 2.00-1.93 (m, 1H), 1.76-1.64 (m, 6H), 1.56-1.46 (m, 1H), 1.35-1.30 (m, 1H), 1.26-1.21 (m, 3H).

### Example 78—Synthesis of Compound I-94

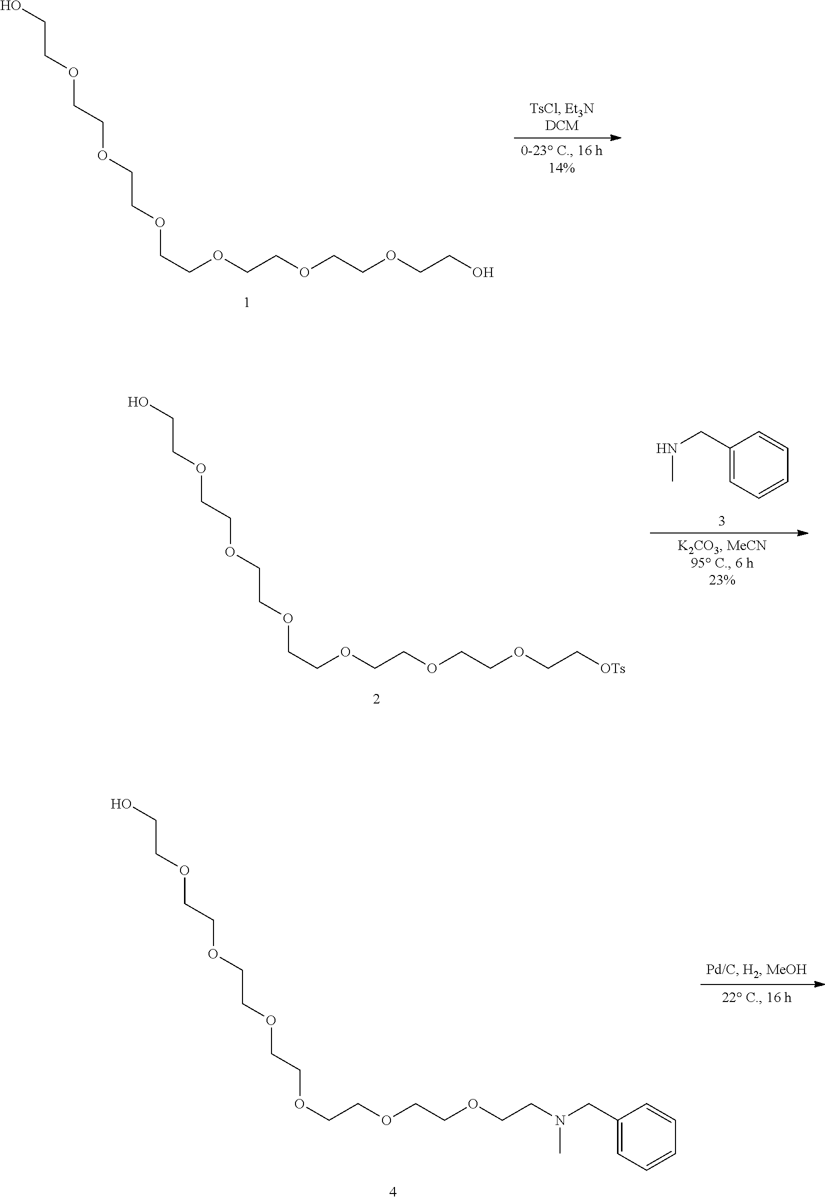

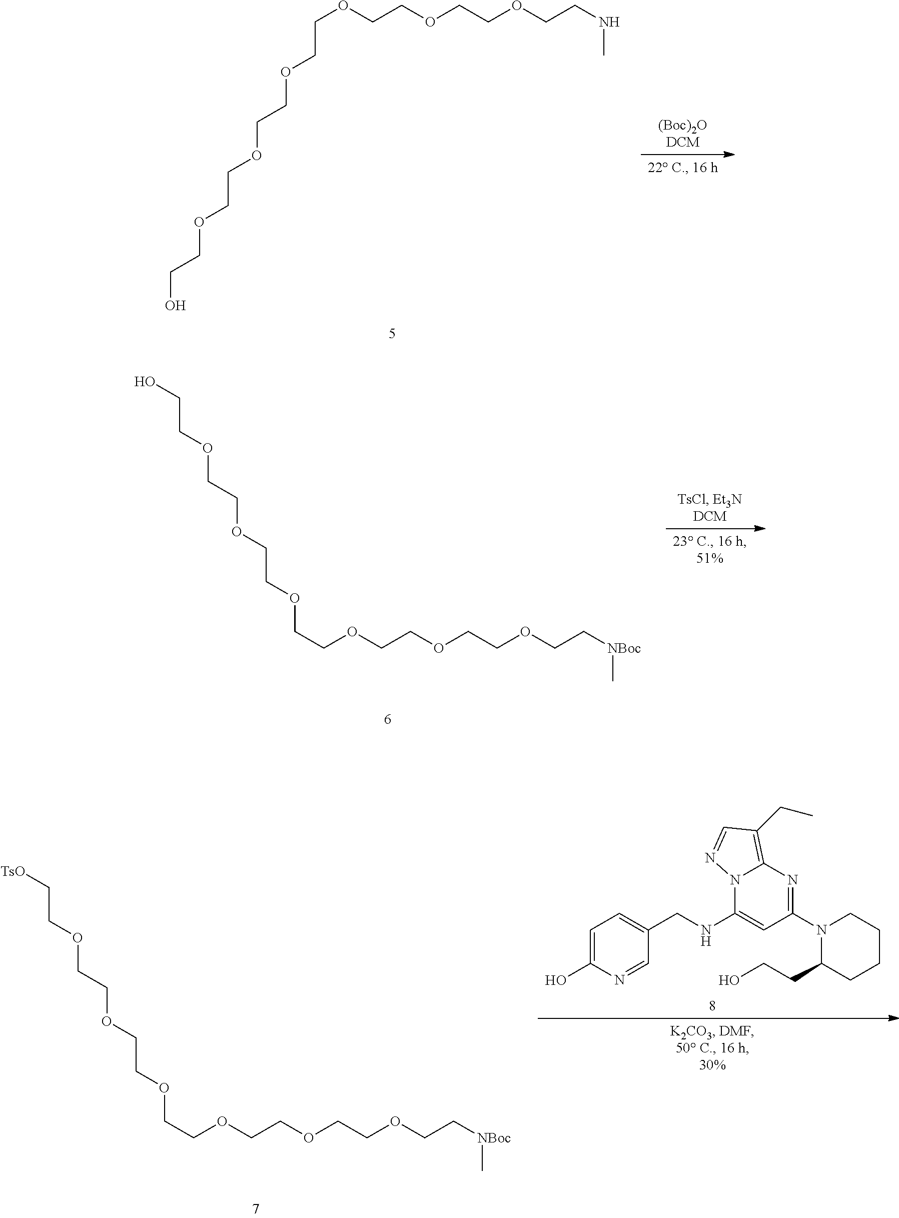

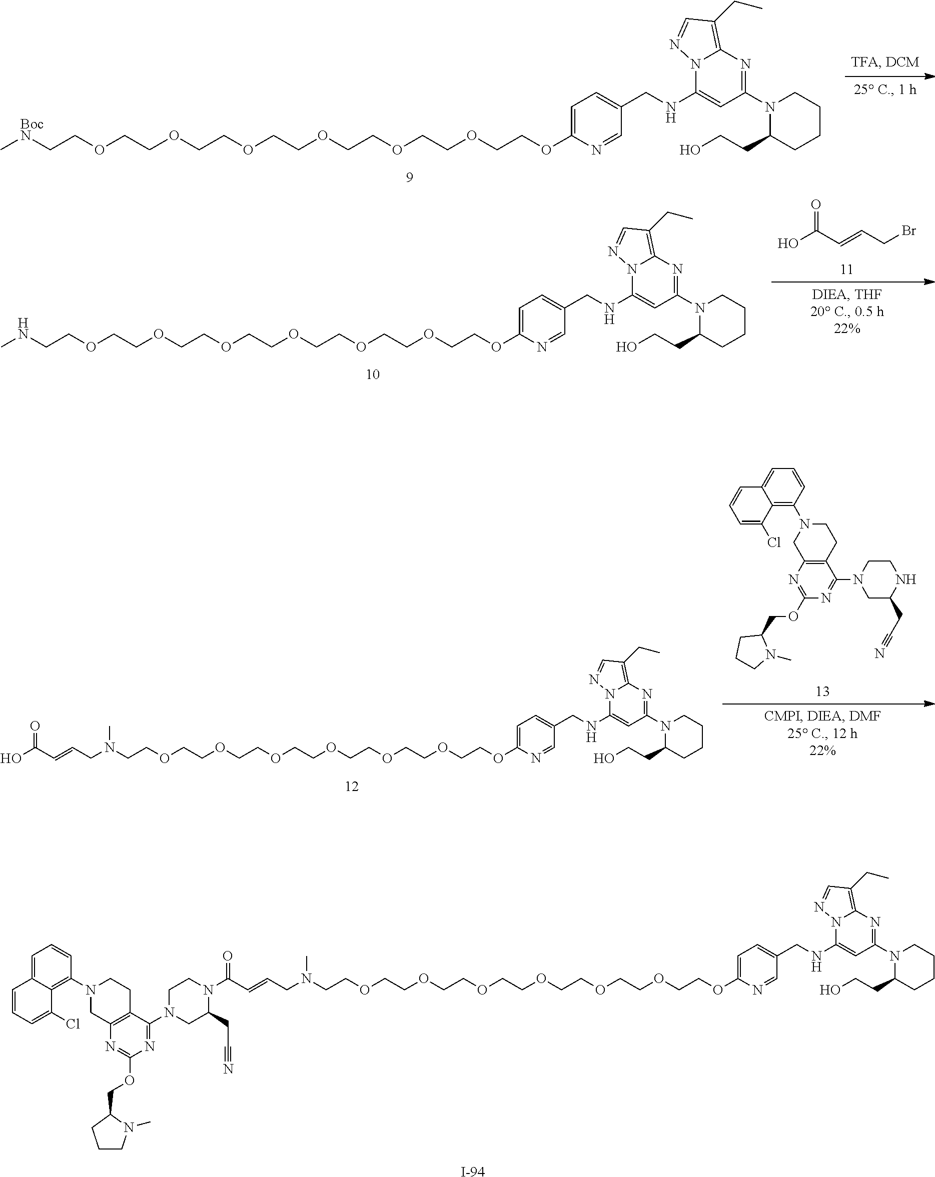

General Information: Synthetic route of compound 13 is reported in WO2021/108683, 2021, A1.

Step 1. To a solution of 3-[2-[2-[2-[2-[2-(2-hydroxyethoxy)ethoxy]ethoxy]ethoxy]ethoxy]ethoxy]propan-1-ol (5.00 g, 14.6 mmol, 1.0 equiv) in DCM (50 mL) was added Et₃N (4.46 g, 44.0 mmol, 6.13 mL, 3.0 equiv) and TsCl (1.14 g, 16.1 mmol, 1.1 equiv) in DCM(20 ml) at 0° C., then the mixture was stirred at 22° C. for 16 h. The reaction mixture was diluted with H₂O 30 mL and extracted with DCM (30 mL×3). The combined organic layers were washed with brine (30 mL), dried over Na₂SO₄, filtered and concentrated under reduced pressure to give a residue. Some of the residue was purified by flash silica gel chromatography (ISCO@; 40 g SepaFlash@ Silica Flash Column, Eluent of 0-10% Ethyl acetate/Petroleum ethergradient @ 40 m/min) to give 2-[2-[2-[2-[2-[2-(2-hydroxyethoxy) ethoxy]ethoxy]ethoxy]ethoxy]ethoxy]ethyl 4-methylbenzenesulfonate (1.2 g, 6.14 mmol, 14% yield, 82% purity) as a colorless oil. Some of the residue was purified by prep-HPLC (column: YMC Triart C18 70×250 mm×7 m; mobile phase: [water(NH₄HCO₃)-ACN]; B%: 25%-55%, 15 min) to give 2-[2-[2-[2-[2-[2-(2-hydroxyethoxy)ethoxy]ethoxy]ethoxy]ethoxy]ethoxy]ethyl 4-methylbenzenesulfonate (1.2 g, 7.14 mmol, 48% yield, 98% purity) as a colorless oil. ¹H NMR: ¹H NMR (400 MHz, CDCl₃) δ=7.79 (d, J=8.4 Hz, 2H), 7.34 (d, J=8.0 Hz, 2H), 4.17-4.13 (m, 2H), 3.72-3.57 (m, 26H), 2.44 (s, 3H).

Step 2. To a solution of 2-[2-[2-[2-[2-[2-(2-hydroxyethoxy)ethoxy]ethoxy]ethoxy]ethoxy]ethoxy]ethyl 4-methylbenzenesulfonate (2.00 g, 4.16 mmol, 1.0 equiv) in CH₃CN (10 mL) was added K₂CO₃ (1.15 g, 8.32 mmol, 2.0 equiv) and N-methyl-1-phenyl-methanamine (756 mg, 6.24 mmol, 805 μL, 1.5 equiv). The mixture was stirred at 95° C. for 6 h. The mixture was filtered and concentrated to give a residue. The residue was purified by flash silica gel chromatography (ISCO@; 20 g SepaFlash@ Silica Flash Column, Eluent of 0-10% Methanol/Dichloromethane @ 50 mL/min) to give 2-[2-[2-[2-[2-[2-[2-[benzyl(methyl) amino]ethoxy]ethoxy]ethoxy]ethoxy]ethoxy]ethoxy]ethanol (500 mg, 955 μmol, 23% yield, 82% purity) as a light yellow oil. ¹H NMR: ¹H NMR (400 MHz, CDCl₃) δ=7.39-7.31 (m, 5H), 3.83-3.78 (m, 1H), 3.77-3.73 (m, 2H), 3.73-3.61 (m, 23H), 3.59 (s, 2H), 2.66 (t, J=6.0 Hz, 2H), 2.50-2.49 (brs, 1H), 2.29 (s, 3H).

Step 3. To a mixture of 2-[2-[2-[2-[2-[2-[2-[benzyl(methyl)amino]ethoxy]ethoxy]ethoxy]ethoxy]ethoxy]ethoxy]ethanol (500 mg, 1.16 mmol, 1.0 equiv) in MeOH(15 mL) was added Pd/C (500 mg, 10% purity) in MeOH(10 mL) under N₂. The suspension was degassed under vacuum and purged with H₂ several times. The mixture was stirred under H₂ (15 psi) at 22° C. for 16 h. The reaction mixture was filtered and the filrate was concentrated to give 2-[2-[2-[2-[2-[2-[2-(methylamino) ethoxy]ethoxy]ethoxy]ethoxy]ethoxy]ethoxy]ethanol (395 mg, crude) as light yellow oil.

Step 4. To a solution of 2-[[2[2-[2-[2-[2-(methylamino)ethoxy]ethoxy]ethoxy]ethoxy]ethoxy]ethoxy]ethanol (395 mg, 1.16 mmol, 1.0 equiv) in DCM (4 mL) was added Boc₂0 (279 mg, 1.28 mmol, 294 μL, 1.1 equiv). The mixture was stirred at 23° C. for 16 hr. The reaction was filtered and concentrated to give a residue. The residue tert-butyl N-[2-[2-[2-[2[2-[2-2(2-hydroxyethoxy)ethoxy]ethoxy]ethoxy]ethoxy]ethoxy]ethyl]-N-methyl-carbamate (500 mg, crude) as a colorless oil was used into next step without purification.

Step 5. To a solution of tert-butyl N-[2-[2-[2-[2-[2-[2-(2-hydroxyethoxy)ethoxy]ethoxy]ethoxy]ethoxy]ethoxy]ethyl]-N-methyl-carbamate (500 mg, 1.14 mmol, 1.0 equiv) in DCM (4 mL) was added Et₃N (287 mg, 2.84 mmol, 395 μL, 2.5 equiv) and TosCl (260 mg, 1.37 mmol, 1.2 equiv). The mixture was stirred at 23° C. for 16 h. The reaction was filtered and concentrated to give a residue. The residue was purified by flash silica gel chromatography (ISCO®; 12 g SepaFlash® Silica Flash Column, Eluent of 0-10% Ethyl acetate/Petroleum ethergradient @ 30 mL/min) to give 2-[[2[2-[2-[2-[2-[tert-butoxycarbonyl(methyl)amino]ethoxy]ethoxy]ethoxy]ethoxy]ethoxy]ethoxy]ethyl 4-methylbenzenesulfonate (360 mg, 588 μmol, 51% yield, 97% purity) as a colorless oil. ¹H NMR: ¹H NMR (400 MHz, CDCl₃) δ=7.83-7.73 (m, 2H), 7.37-7.28 (m, 2H), 3.71-3.48 (m, 28H), 2.89 (m, 3H), 2.44 (m, 3H), 1.46-1.41 (m, 9H).

Step 6. To a solution of 2-[[2[2-[2-[2-[2-[tert-butoxycarbonyl(methyl)amino]ethoxy]ethoxy]ethoxy]ethoxy]ethoxy]ethoxy]ethyl 4-methylbenzenesulfonate (292 mg, 491 μmol, 1.3 equiv) in MeCN (5 mL) was added K₂CO₃ (156 mg, 1.13 mmol, 3.0 equiv) and 5-[[[3-ethyl-5-[(2S)-2-(2-hydroxyethyl)-1-piperidyl]pyrazolo [1,5-a]pyrimidin-7-yl]amino]methyl]pyridin-2-ol (150 mg, 378 μmol, 1.0 equiv). The mixture was stirred at 50° C. for 16 h. The reaction was filtered and concentrated to give a residue. The residue was purified by prep-HPLC (column: Phenomenex C18 150×25 mm×10%m; mobile phase: [water(NH₄HCO₃)-ACN]; B%: 43%-73%, 8 min) to give tert-butyl N-[2-[2-[2-[2-[2-[2-[2-[[5-[[[3-ethyl-5-[(2S)-2-(2-hydroxyethyl)-1-piperidyl]pyrazolo[ 1,5-a]pyrimidin-7-yl]amino]methyl]-2-pyridyl]oxy]ethoxy]ethoxy]ethoxy]ethoxy]ethoxy]ethoxy]ethyl]-N-methyl-carbamate (93 mg, 113 μmol, 30% yield, 99% purity)as a brown gum. ¹H NMR: ¹H NMR (400 MHz, CDCl₃) δ=8.17-8.13 (m, 1H), 7.64 (s, 1H), 7.61 (dd, J=2.8, 8.4 Hz, 1H), 6.81 (d, J=8.8 Hz, 1H), 6.46-6.20 (m, 1H), 5.30 (s, 1H), 5.09 (m, 1H), 4.50-4.46 (m, 2H), 4.45 (d, J=5.6 Hz, 2H), 3.86 (dd, J=4.0, 5.6 Hz, 2H), 3.74-3.59 (m, 26H), 3.41-3.31 (m, 3H), 3.10-3.00 (m, 1H), 2.91 (s, 3H), 2.61 (m, 2H), 1.71-1.60 (m, 1OH), 1.46 (s, 9H).

Step 7. To a solution of tert-butyl N-[2-[2-[2-[2-[2-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethyl]-N-methyl-carbamate (90 mg, 110 μmol, 1.0 equiv) in DCM (1 mL) was added TFA (1.54 g, 13.5 mmol, 1 mL, 123.0 equiv). The mixture was stirred at 25° C. for 1 h. The mixture was concentrated under vacuum. Compound 2-[(2S)-1-[3-ethyl-7-[[6-[1[2-[2-[2-[2-[2-[2-(methylamino)ethoxy]ethoxy]ethoxy]ethoxy]ethoxy]ethoxy]ethoxy]-3-pyridyl]methylamino]pyrazolo[1,5-a]pyrimidin-5-yl]-2-piperidyl]ethanol (90 mg, 108.18 μmol, 98.33% yield, TFA) was obtained as a yellow oil.

Step 8. To a solution of 2-[(2S)-1-[3-ethyl-7-[[6-[2-[2-[2-[2-[2-[2-[2-(methylamino)ethoxy]ethoxy]ethoxy]ethoxy]ethoxy]ethoxy]ethoxy]-3-pyridyl]methylamino]pyrazolo[1,5-a]pyrimidin-5-yl]-2-piperidyl]ethanol (60 mg, 83.58 μmol, 1.0 equiv) in THF (1 mL) was added DIPEA (32.4 mg, 251 μmol, 44 μL, 3.0 equiv).and stirred at 20° C. for 0.5 h. Then (E)-4-bromobut-2-enoic acid (13.8 mg, 83.6 μmol, 1.0 equiv) was added into the reaction mixture was stirred at 20° C. for 12 h. The reaction mixture was diluted with water (15 mL) and extracted with ethyl acetate (15 mL×2). The combined organic layers were washed with brine (10 mL×2), dried over anhydrous sodium sulfate, filtered and concentrated under reduced pressure to give a residue. The residue was purified by preparative-TLC (10% methanol in dichloromethane) (dichloromethane: methanol=5:1, Rf=0.2). Compound (S,E)-1-((5-(((3-ethyl-5-(2-(2-hydroxyethyl)piperidin-1-yl)pyrazolo [1,5-a]pyrimidin-7-yl)amino)methyl)pyridin-2-yl)oxy)-21-methyl-3,6,9,12,15,18-hexaoxa-21-azapentacos-23-en-25-oic acid (15 mg, 18.7 μmol, 22% yield) was obtained as a yellow oil.

Step 9. To a solution of 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]piperazin-2-yl]acetonitrile (12.08 mg, 18.70 μmol, 1.0 equiv, TFA), CMPI (9.56 mg, 37.41 μmol, 2.0 equiv) and DIPEA (12.09 mg, 93.52 μmol, 16.29 μL, 5.0 equiv) in DMF (0.5 mL) was added (S,E)-1-((5-(((3-ethyl-5-(2-(2-hydroxyethyl)piperidin-1-yl)pyrazolo[1,5-a]pyrimidin-7-yl)amino)methyl) pyridin-2-yl)oxy)-21-methyl-3,6,9,12,15,18-hexaoxa-21-azapentacos-23-en-25-oic acid (15 mg, 18.7 μmol, 1.0 equiv). The reaction mixture was stirred at 25° C. for 12 h. The mixture was purified by semi-preparative reverse phase HPLC (24-54% acetonitrile 0.225% formic acid in water, 10.5 min). Compound 2-((S)-4-(7-(8-chloronaphthalen-1-yl)-2-(((S)-1-methylpyrrolidin-2-yl)methoxy) -5,6,7,8-tetrahydropyrido[3,4-d]pyrimidin-4-yl)-1-((E)-1-((5-(((3-ethyl-5-((S)-2-(2-hydroxyethyl) piperidin-1-yl)pyrazolo[1,5-a]pyrimidin-7-yl)amino)methyl)pyridin-2-yl)oxy)-21-methyl-3,6,9,12,15,18-hexaoxa-21-azapentacos-23-en-25-oyl)piperazin-2-yl)acetonitrile (6.02 mg, 4.20 μmol, 22% yield, 95.1% purity, FA) was obtained as a yellow solid. LC-MS: MS (ES⁺): RT=2.370 min, m/z=658.2 [M/2+H]+. LCMS Method: 05. ¹H NMR: ¹H NMR (400 MHz, CD₃OD) 6=8.52 (brs, 1H), 8.18 (m, 1H), 7.82-7.80 (d, J=8.4 Hz, 1H), 7.68-7.66 (d, J=8.4 Hz, 1H), 7.63 (m, 2H), 7.52-7.48 (m, 2H), 7.38-7.30 (m, 2H), 6.87-6.77 (m, 3H), 5.52 (s, 1H), 5.08 (m, 1H), 4.78 (m, 4H), 4.52 (m, 2H), 4.50 (m, 4H), 4.49 (m, 2H), 4.45 (m, 1H), 4.41 (m, 1H), 4.40 (m, 2H), 3.81 (m, 2H), 3.66 (m, 1H), 3.65 (m, 5H), 3.60 (m, 25H), 3.50 (m, 2H), 2.98 (m, 1OH), 2.55 (m, 4H), 2.30 (m, 1H), 2.00 (m, 4H), 1.71 (m, 6H), 1.50 (m, 1H), 1.24-1.20 (t, J=8.0 Hz, 1H).

### Example 79—Synthesis of Compound I-95

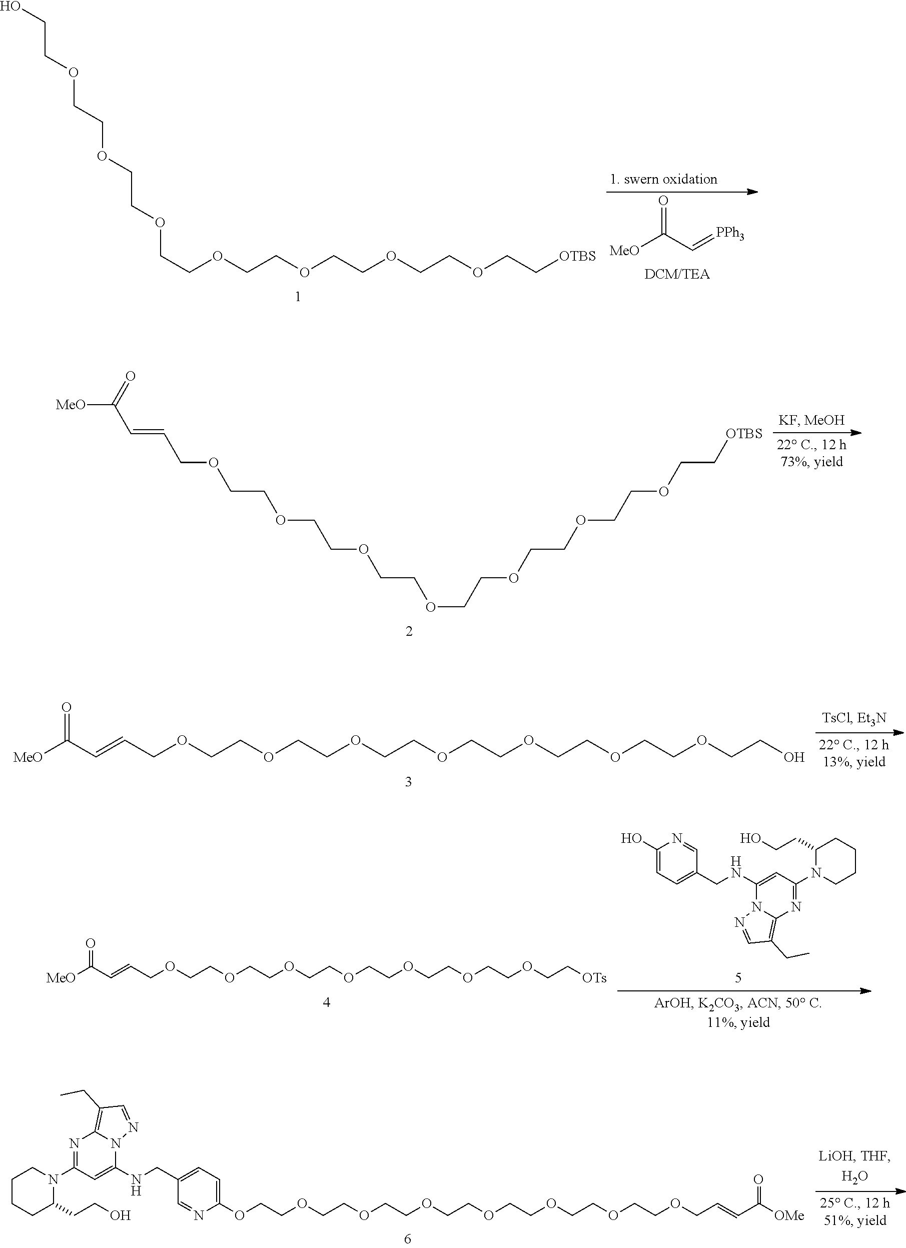

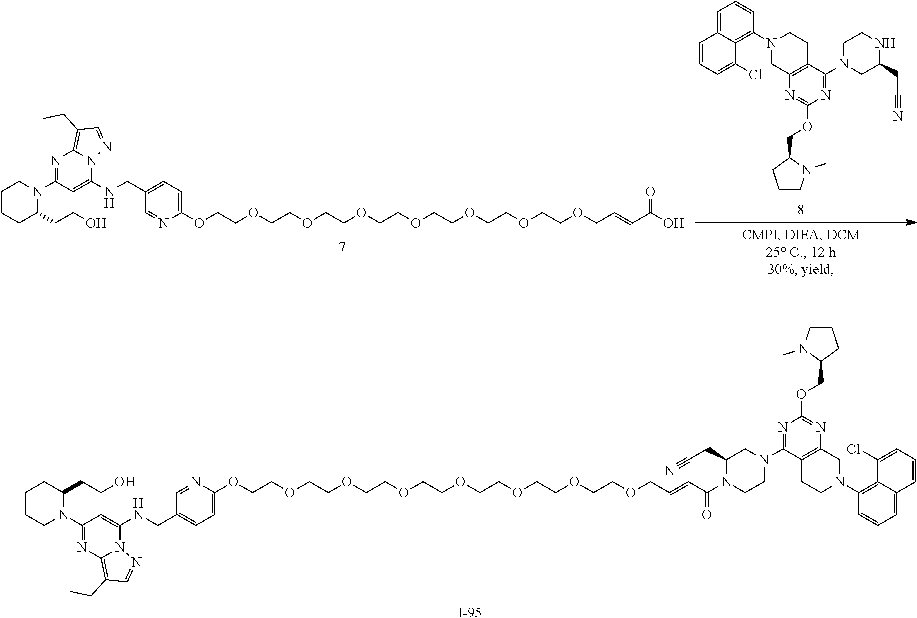

General Information: Synthetic route of compound 8 is reported in WO2021/108683, 2021, A1.

Step 1. To a solution of oxalyl dichloride (3.8 g, 29.7 mmol, 2.6 mL, 2.0 equiv) in DCM (90 mL) was added dropwise methylsulfinylmethane (4.6 g, 59 mmol, 4.6 mL, 4.0 equiv) at −78° C. The mixture was stirred at −78° C. for 0.5 h. Then to the reaction mixture was added dropwise a solution of 2-[[2[2-[2-[2-[2-[2-[tert-butyl(dimethyl)silyl]oxyethoxy]ethoxy]ethoxy]ethoxy]ethoxy]ethoxy]ethoxy]ethanol (7.2 g, 14.9 mmol, 1 equiv) in DCM (20 mL) and the mixture was stirred at −78° C. for 0.5 h. To the reaction mixture was added dropwise TEA (12.0 g, 118 mmol, 16.5 mL, 8.0 equiv) at −78° C. under N₂ for 0.5 h. Then the resulting mixture was stirred at 22° C. under N₂ for 2 h. The reaction mixture was added methyl 2-(triphenyl-k5-phosphanylidene)acetate (7.0 g, 21 mmol, 1.5 equiv) in DCM (80 mL). Then the resulting mixture was stirred at 23° C. under N₂ for 12 h. The reaction mixture was filtered and concentrated under reduced pressure to give a residue. The crude product was purified by reversed-phase HPLC(column: Phenomenex luna C18 250*50 mm*15 m; mobile phase: [water(NH₄HCO₃)-ACN]; B%: 50%-80%, 20 min) to give the methyl (E)-4-[2-[2-[2-[2-[2-[2-[2[tertbutyl(dimethyl)silyl]oxyethoxy]ethoxy]ethoxy]ethoxy]ethoxy]ethoxy]ethoxy]but-2-enoate (1.3 g, 1.9 mmol, 13% yield) as yellow oil. ¹H NMR: (400 MHz, CDCl₃) δ=6.95-6.55 (m, 1H), 6.11-6.07 (m, 1H), 4.30-4.31 (m, 2H), 3.86-3.76 (m, 5H), 3.66-3.65 (m, 24H), 3.50-3.48 (m, 2H), 1.03 (s, 9H), 0.60 (s, 6H).

Step 2. To a solution of methyl (E)-4-[2-[2-[2-[2-[2-[2-[2-[tert-butyl(dimethyl) silyl]oxyethoxy]ethoxy]ethoxy]ethoxy]ethoxy]ethoxy]ethoxy]but-2-enoate (1.3 g, 2.4 mmol, 1.0 equiv) in MeOH (10 mL) was added KF (700 mg, 12.1 mmol, 282 μL, 5.0 equiv) at 22° C. Then the resulting mixture was stirred at 22° C. under N₂ for 12 h. The crude reaction mixture was concentrated under reduced pressure to remove solvent. The residue was diluted with H₂O (50 mL) and extracted with DCM (30 mL×3). The combined organic layers were washed with brine (50 mL×2), dried over Na₂SO₄, filtered and concentrated under reduced pressure to give a residue and was purified by the flash silica gel chromatography (ISCO®; 24 g SepaFlash® Silica Flash Column, Eluent of 0-30% Ethyl acetate/Methanol gradient @ 80 mL/min) to give the methyl (E)-4-[2-[2-[2-[2-[2-[2-(2-hydroxyethoxy)ethoxy]ethoxy]ethoxy]ethoxy]ethoxy]ethoxy]but-2-enoate (1.0 g, 1.8 mmol, 73% yield) as yellow oil.

Step 3. To a solution of methyl (E)-4-[2-[2-[2-[2-[2-[2-(2-hydroxyethoxy) ethoxy]ethoxy]ethoxy]ethoxy]ethoxy]ethoxy]but-2-enoate (1 g, 2.36 mmol, 1.0 equiv) in DCM (5 mL) was added TEA (715 mg, 7.1 mmol, 983 μL, 3.0 equiv) and 4-methylbenzenesulfonyl chloride (494 mg, 2.6 mmol, 1.1 equiv) at 22° C. Then the resulting mixture was stirred at 22° C. under N₂ for 12 h. The reaction mixture was filtered and concentrated under reduced pressure to give a residue. The residue was purified by flash silica gel chromatography (column: YMC Triart C18 250*50 mm*7 m; mobile phase: [water(NH4HCO₃)-ACN]; B%: 30%-60%, 20 min) to give the methyl (E)-4-[21[2-[2-[2-[12-[2-(p-tolylsulfonyloxy)ethoxy]ethoxy]ethoxy]ethoxy]ethoxy]ethoxy ethoxy]but-2-enoate (180 mg, 305 μmol, 13% yield) as yellow oil.

Step 4. To a solution of 5-[[[3-ethyl-5-[(2S)-2-(2-hydroxyethyl)-1-piperidyl]pyrazolo [1,5-a]pyrimidin-7-yl]amino]methyl]pyridin-2-ol (123 mg, 311 μmol, 1.0 equiv) in ACN (1 mL) was added K₂CO₃ (257 mg, 1.9 mmol, 6.0 equiv) and methyl (E)-4-[2-[2-[2-[2-[2-[2-[2-(p-tolylsulfonyloxy)ethoxy]ethoxy]ethoxy]ethoxy]ethoxy]ethoxy]ethoxy]but-2-enoate (180 mg, 311 μmol, 1.0 equiv). Then the resulting mixture was stirred at 50° C. for 12 h. The combined reaction mixture was filtered and concentrated under reduced pressure to give a residue. The residue was purified by prep-HPLC (column: Phenomenex C18 150*25 mm*10 m; mobile phase: [water(NH₄HCO₃)-ACN]; B%: 36%-66%, 5 min) to give the methyl (E)-4-[2-[2-[2-[2-[2-[2-[2-[[5-1[[[3-ethyl-5-1[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethoxy]but-2-enoate (35 mg, 42 μmol, 11% yield) as yellow oil.

Step 5. A mixture of methyl (E)-4-[2-[2-[2-[2-[2-[2-[2-[[5-[[[3-ethyl-5-[(2S)-2-(2-hydroxyethyl)-1-piperidyl]pyrazolo[1,5-a]pyrimidin-7-yl]amino]methyl]-2pyridyl]oxy]ethoxy]ethoxy]ethoxy]ethoxy]ethoxy]ethoxy]ethoxy]but-2-enoate (60 mg, 74 μmol, 1.0 equiv), LiOH·H₂O (6 mg, 149 μmol, 2.0 equiv) in THF (3 mL), H₂O (1 mL) was degassed and purged with N₂ for 3×, and then the mixture was stirred at 25° C. for 12 h under N₂ atmosphere. The residue was purified by the prep-HPLC (column: Phenomenex C18 150*25 mm*10 μm; mobile phase: [water(NH₄HCO₃)-ACN]; B%: 22%-52%, 8 min) to give the (E)-4-[2-[2-[2-[2-[2-[2-[2-[[5-[[[3-ethyl-5-[(2S)-2-(2-hydroxyethyl)-1-piperidyl]pyrazolo[1,5a]pyrimidin7-yl]amino]methyl]-2-pyridyl]oxy]ethoxy]ethoxy]ethoxy]ethoxy]ethoxy]ethoxy]ethoxy]but-2-enoic acid (30 mg, 38 μmol, 51% yield) as a colorless oil.

Step 6. A mixture of (E)-4-[2-[2-[2-[2-[2-[2-[2-[[5-[[[3-ethyl-5-[(2S)-2-(2-hydroxyethyl)-1-piperidyl]pyrazolo[ 1,5-a]pyrimidin-7-yl]amino]methyl]-2-pyridyl]oxy]ethoxy]ethoxy]ethoxy]ethoxy]ethoxy]ethoxy]ethoxy]but-2-enoic acid (25 mg, 31 μmol, 1.0 equiv), 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]piperazin-2-yl]acetonitrile (17 mg, 31 μmol, 1.0 equiv), DIPEA (12 mg, 95 μmol, 3.0 equiv), CMPI (10 mg, 38 μmol, 1.2 equiv) in DCM (1 mL) was degassed and purged with N₂ for 3×, and then the mixture was stirred at 25° C. for 2 h under N₂ atmosphere. The mixture was filtered and concentrated to get the residue. The residue was purified by the prep-HPLC(column: Phenomenex C18 150*25 mm*10 m; mobile phase: [water(NH₄HCO₃)-ACN]; B%: 57%-87%, 8 min) to give the 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-[(E)-4-[2-[12[2-[2-[2-[2-2-[[5-[[[3-ethyl-5-[(2S)-2-(2-hydroxyethyl)-1-piperidyl]pyrazolo[1,5a]pyrimidin7-yl]amino]methyl]2pyridyl]oxy]ethoxy]ethoxy]ethoxy]ethoxy]ethoxy]ethoxy]ethoxy]but-2-enoyl]piperazin-2-yl]acetonitrile (13 mg, 10 μmol, 30% yield, 95% purity) as a white solid. LC-MS: MS (ES⁺): RT=2.576 min, m/z=652.1 [1/2M+H⁺]. LCMS Method: 10. ¹H NMR: (400 MHz, CDCl₃) δ=8.14 (d, J=2.0 Hz, 1H), 7.75 (d, J=8.0 Hz, 1H), 7.68-7.58 (m, 3H), 7.55-7.36 (m, 3H), 7.34 (d, J=8.0 Hz, 1H), 7.27-7.18 (m, 1H), 6.80 (d, J=8.4 Hz, 1H), 6.34-6.25 (m, 1H), 6.23-6.11 (m, 1H), 5.29 (s, 1H), 5.17-4.95 (m, 2H), 4.63-4.53 (m, 2H), 4.49-4.40 (m, 5H), 4.17-3.84 (m, 1OH), 3.72-3.59 (m, 22H), 3.42-3.01 (m, 1OH), 2.87-2.47 (m, 1OH), 2.34-2.28 (m, 1H), 2.15-2.03 (m, 3H), 1.72-1.49 (m, 8H), 1.28-1.23 (m, 3H).

### Example 80—Synthesis of Compound I-96

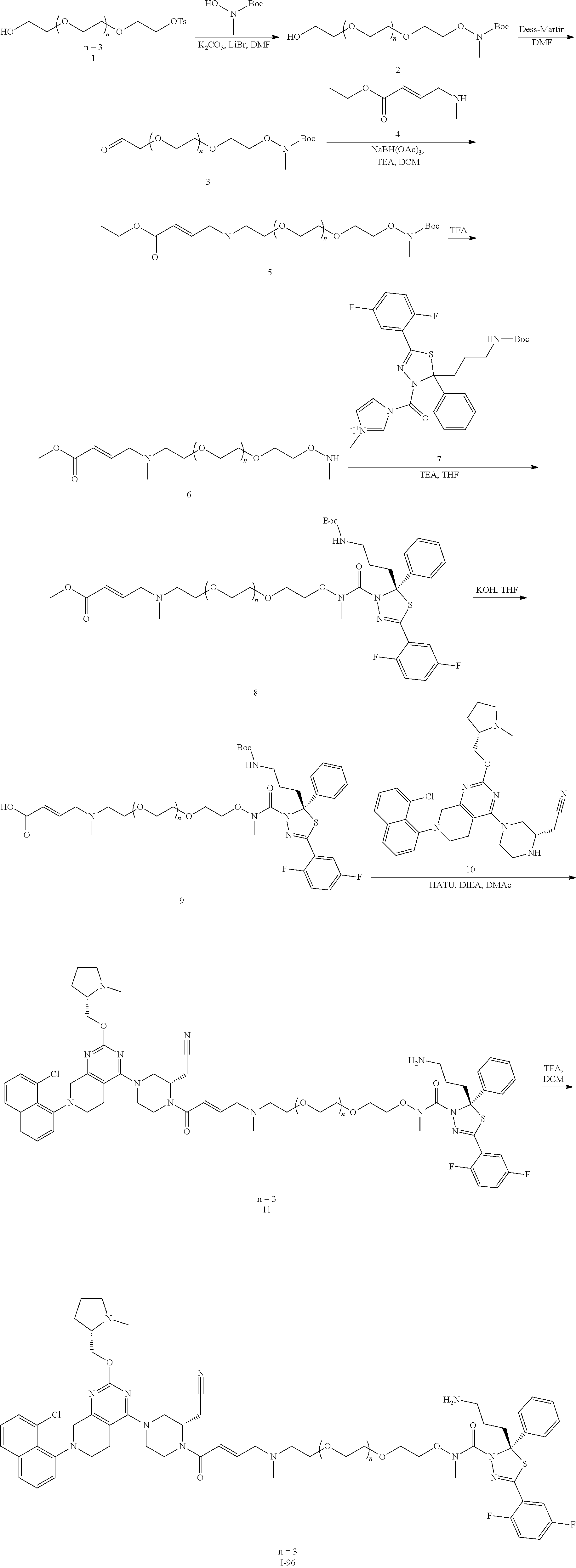

General Information: Compound 10 is a known compound from WO2021/108683, 2021, A1. Compound 7 is a known compound from US2006/247178.

Step 1. To a solution of 2-[2-[2-[2-(2-hydroxyethoxy)ethoxy]ethoxy]ethoxy]ethyl 4-methylbenzenesulfonate (2.8 g, 7.13 mmol, leq) and tert-butyl N-hydroxy-N-methyl-carbamate (1.57 g, 10.7 mmol, 1.5 equiv) in DMF (12 mL) was added K₂CO₃ (2.96 g, 21.4 mmol, 3 equiv) and LiBr (13 mg, 143 μmol, 0.02 equiv). The mixture was stirred at 100° C. for 2 h and concentrated. The residue was purified by prep-HPLC (column: Waters Xbridge 150*25 mm*5 m; mobile phase: [water(NH₄HCO₃)-ACN]; B%: 10%-40%, 10 min) to give tert-butyl N-[2-[2-[2-[2-(2-hydroxyethoxy)ethoxy]ethoxy]ethoxy]ethoxy]-N-methyl-carbamate (600 mg, 23% yield). ¹H NMR (400 MHz, CD₃OD): δ 3.99-3.97 (m, 2H), 3.69-3.63 (m, 16H), 3.60-3.55 (m, 2H), 3.10 (s, 3H), 1.49 (s, 9H).

Step 2. To a solution of tert-butyl N-[2-[2-[2-[2-(2-hydroxyethoxy)ethoxy]ethoxy]ethoxy]ethoxy]-N-methyl-carbamate (300 mg, 816 μmol, 1 equiv) in DMF (4 mL) was added Dess-Martin (866 mg, 2.04 mmol, 2.5 equiv). The mixture was stirred at 25° C. for 1 h and concentrated. The crude product was used directly for next step.

Step 3. To a solution of tert-butyl N-methyl-N-[2-[2-[2-[2-(2-oxoethoxy)ethoxy]ethoxy]ethoxy]ethoxy]carbamate (290 mg, 794 μmol, 1 equiv) and ethyl (E)-4-(methylamino)but-2-enoate (204 mg, 794 μmol, 1 equiv, TFA salt) in DCM (20 mL) was added NaBH(OAc)₃ (841 mg, 4 mmol, 5 eq) and TEA (402 mg, 4 mmol, 5 equiv). The mixture was stirred at 25° C. for 0.5 h and concentrated. The residue was purified by prep-HPLC (column: Waters Xbridge 150*25 mm*5 m; mobile phase: [water(NH₄HCO₃)-ACN]; B%: 32%-62%, 10 min) to give ethyl (E)-4-[2-[2-[2-[2-[2-[tert-butoxycarbonyl(methyl)amino]oxyethoxy]ethoxy]ethoxy]ethoxy]ethyl-methylamino]but-2-enoate (200 mg, 51% yield).

Step 4. To a solution of ethyl (E)-4-[2-[2-[2-[2-[2-[tert-butoxycarbonyl(methyl)amino]oxyethoxy]ethoxy]ethoxy]ethoxy]ethyl-methylamino]but-2-enoate (200 mg, 406 μmol, 1 equiv) in DCM (5 mL) was added TFA (0.5 mL). The mixture was stirred at 25° C. for 0.5 h and concentrated. The residue was used directly for next step.

Step 5. To a solution of ethyl (E)-4-[methyl-[2-[2-[2-[2-[2-(methylaminooxy) ethoxy]ethoxy]ethoxy]ethoxy]ethyl]amino]but-2-enoate (200 mg, 395 μmol, 1 equiv, TFA salt) in THF (2 mL) was added TEA (240 mg, 2.37 mmol, 6 equiv), then tert-butyl N-[3-[5-(2,5-difluorophenyl)-3-(3-BLAH-3-methyl-imidazole-1-carbonyl)-2-phenyl-1,3,4-thiadiazol-2-yl]propyl]carbamate (317 mg, 474 μmol, 1.2 equiv) was added. The mixture was stirred at 25° C. for 12 h and concentrated. The residue was purified by prep-TLC (SiO₂, DCM: MeOH=10:1) to give ethyl (E)-4-[2-[2-[2-[2-[2-[[2-[3-(tert-butoxycarbonylamino)propyl]-5-(2,5-difluorophenyl)-2-phenyl-1,3,4-thiadiazole-3-carbonyl]-methyl-amino]oxyethoxy]ethoxy]ethoxy]ethoxy]ethyl-methyl-amino]but-2-enoate (240 mg, 72% yield).

Step 6. To a solution of ethyl (E)-4-[2-[2-[2-[2-[2-[[2-[3-(tert-butoxycarbonylamino) propyl]-5-(2,5-difluorophenyl)-2-phenyl-1,3,4-thiadiazole-3-carbonyl]-methyl-amino]oxyethoxy]ethoxy]ethoxy]ethoxy]ethyl-methyl-amino]but-2-enoate (230 mg, 270 μmol, 1 equiv) in THF (2 mL) was added KOH (0.5 M, 1.62 mL, 3 equiv). The mixture was stirred at 25° C. for 12 h. The mixture was adjusted pH to 6 with HCl (0.5 M, 3 mL) and concentrated. The crude product was triturated with DCM at 25° C. for 1 0 min to give (E)-4-[2-[2-[2-[2-[2-[[2-[3-(tert-butoxycarbonylamino)propyl]-5-(2,5-difluorophenyl)-2-phenyl-1,3,4-thiadiazole-3-carbonyl]-methyl-amino]oxyethoxy]ethoxy]ethoxy]ethoxy]ethyl-methyl-amino]but-2-enoic acid (210 mg, 94% yield).

Step 7. To a solution of (E)-4-[2-[2-[2-[2-[2-[[2-[3-(tert-butoxycarbonylamino)propyl]-5-(2,5-difluorophenyl)-2-phenyl-1,3,4-thiadiazole-3-carbonyl]-methylamino]oxyethoxy]ethoxy]ethoxy]ethoxy]ethyl-methyl-amino]but-2-enoic acid (163 mg, 198 μmol, 1 equiv) and 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]piperazin-2-yl]acetonitrile (143 mg, 198 μmol, 1 equiv) in DMAc (2 mL) was added HATU (165 mg, 435 μmol, 2.2 equiv) and DIEA (128 mg, 989 μmol, 5 equiv). The mixture was stirred at 25° C. for 1 h and concentrated. The residue was purified by prep-HPLC (column: Phenomenex luna C18 250*50 mm*15 m; mobile phase: [water(FA)-ACN]; B%: 27%-57%, 10 min) to give tert-butyl N-[3-[3-[2-[2-[2-[2-[2-[[(E)-4-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-2-(cyanomethyl)piperazin-1-yl]-4-oxo-but-2-enyl]-methylamino]ethoxy]ethoxy]ethoxy]ethoxy]ethoxy-methyl-carbamoyl]-5-(2,5-difluorophenyl)-2-phenyl-1,3,4-thiadiazol-2-yl]propyl]carbamate (40 mg, 15% yield).

Step 8. To a solution of tert-butyl N-[3-[3-[2-[2-[2-[2-[2-[[(E)-4-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-2-(cyanomethyl)piperazin-1-yl]-4-oxo-but-2-enyl]-methylamino]ethoxy]ethoxy]ethoxy]ethoxy]ethoxy-methyl-carbamoyl]-5-(2,5-difluorophenyl)-2-phenyl-1,3,4-thiadiazol-2-yl]propyl]carbamate (35 mg, 26 μmol, 1 equiv) in DCM (2 mL) was added TFA (0.2 mL). The mixture was stirred at 25° C. for 0.5 h and concentrated. The residue was purified by prep-HPLC (column: Phenomenex luna C18 250*50 mm*15 m; mobile phase: [water(FA)-ACN]; B%: 17%-47%, 10 min) to give 2-(3-aminopropyl)-N-[2-[2-[2-[2-[2-[[(E)-4-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-2-(cyanomethyl)piperazin-1-yl]-4-oxo-but-2-enyl]-methyl amino]ethoxy]ethoxy]ethoxy]ethoxy]ethoxy]-5-(2,5-difluorophenyl)-N-methyl-2-phenyl-1,3,4-thiadiazole-3-carboxamide (19 mg, 54% yield). ¹H NMR (400 MHz, MeOD): δ8.58-8.45 (m, 2H), 7.84-7.82 (m, 1H), 7.70-7.68 (m, 1H), 7.55-7.50 (m, 4H), 7.35-7.30 (m, 3H), 7.30-7.28 (m, 3H), 6.86-6.82 (m, 2H), 5.10-5.02 (m, 2H), 4.88-4.50 (m, 3H), 4.32-4.05 (m, 6H), 3.66-3.49 (m, 23H), 3.55-3.05 (m, 10H), 3.00-2.85 (m, 6H), 2.75-2.45 (m, 4H), 2.45-1.75 (m, 6H). LC-MS: MS (ES′): RT=1.834 min, m/z=1237.4 [M+H⁺]; LCMS method: 25.

### Example 81—Synthesis of Compound I-97

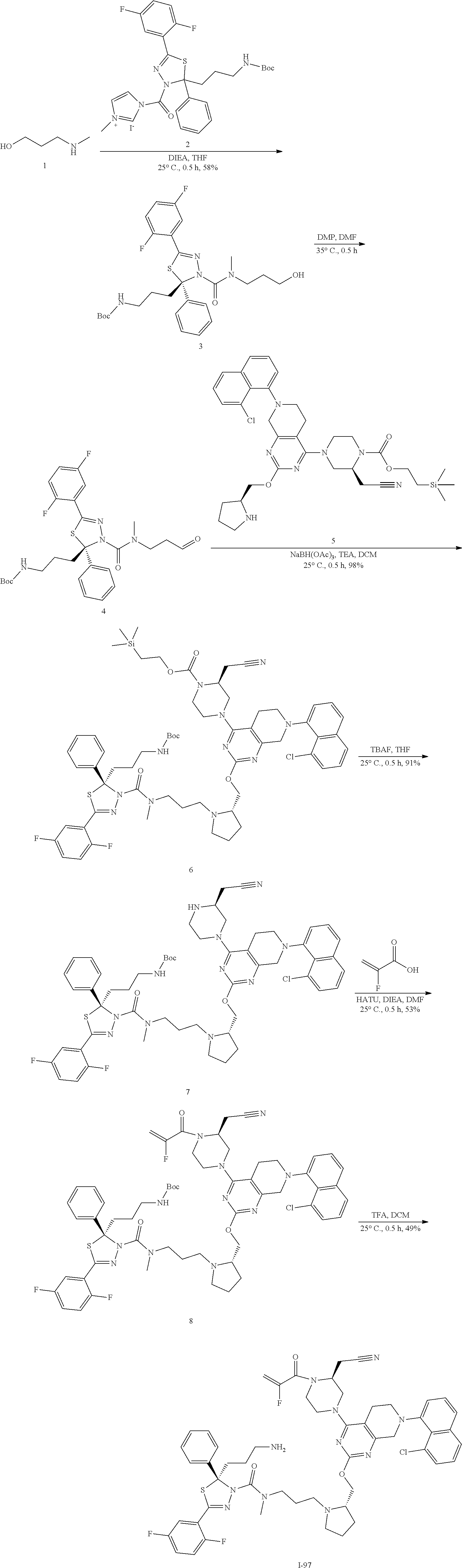

Step 1. To a solution of tert-butyl N-[3-[5-(2,5-difluorophenyl)-3-(3-BLAH-3-methyl-imidazole-1-carbonyl)-2-phenyl-1,3,4-thiadiazol-2-yl]propyl]carbamate (375.5 mg, 560.9 μmol, 1.0 equiv) and 3-(methylamino)propan-1-ol (50 mg, 560.9 μmol, 1.0 equiv) in THF (2 mL) was added DIEA (217.5 mg, 1.8 mmol, 293.1 μL, 3.0 equiv), and then it was stirred at 25° C. for 0.5 h. The reaction mixture was concentrated to afford crude product. The residue was purified by prep-HPLC (column: Waters Xbridge 150*25 mm*5 m; mobile phase: [water (NH₄HCO₃)-ACN]; B%: 54%-84%, 10 min) to give compound tert-butyl N-[3-[(2S)-5-(2,5-difluorophenyl)-3-[3-hydroxypropyl(methyl)carbamoyl]-2-phenyl-1,3,4-thiadiazol-2-yl]propyl]carbamate (180 mg, 58% yield) as a white solid.

Step 2. To a solution of tert-butyl N-[3-[(2S)-5-(2,5-difluorophenyl)-3-[3-hydroxypropyl (methyl) carbamoyl]-2-phenyl-1, 3, 4-thiadiazol-2-yl]propyl]carbamate (40 mg, 72.9 μmol, 1.0 equiv) in DMF (1 mL) was added Dess-Martin (61.8 mg, 145.8 μmol, 45.4 μL, 2.0 equiv). The mixture was stirred at 35° C. for 0.5 h. tert-butyl N-[3-[(2S)-5-(2, 5-difluorophenyl)-3-[methyl (3-oxopropyl) carbamoyl]-2-phenyl-1, 3, 4-thiadiazol-2-yl]propyl]carbamate (39 mg, 98% yield) was obtained as a yellow liquid and used for the next step directly.

Step 3. To a solution of 2-trimethylsilylethyl (2S)-4-[7-(8-chloro-1-naphthyl)-2-[[(2S)-pyrrolidin-2-yl]methoxy]-6,8-dihydro-SH-pyrido[3,4-d]pyrimidin-4-yl]-2(cyanomethyl) piperazine-1-carboxylate (55.4 mg, 71.3 μmol, 1.0 equiv, TFA salt) in DCM (5 mL) was added NaBH(OAc)₃ (151.2 mg, 713.5 μmol, 10.0 equiv) and Et₃N (50.5 mg, 499.4 μmol, 69.5 μL, 7.0 equiv), and then tert-butyl N-[3-[(2S)-5-(2, 5-difluorophenyl)-3-[methyl (3-oxopropyl) carbamoyl]-2-phenyl-1, 3, 4-thiadiazol-2-yl]propyl]carbamate (39 mg, 71.3 μmol, 1.0 equiv) was added. The mixture was stirred at 25° C. for 0.5 h. The reaction mixture was concentrated to afford crude product. The residue was purified by prep-HPLC (column: Phenomenex Synergi C18 150*25 mm*10 m; mobile phase: [water (FA)-ACN]; B%: 49%-79%, 10 min) to give compound 2-trimethylsilylethyl (2S)-4-[2-[[(2S)-1-[3-[[(2S)-2-[3-(tert-butoxycarbonylamino)propyl]-5-(2,5-difluorophenyl)-2-phenyl-1,3,4-thiadiazole-3-carbonyl]-methyl-amino]propyl]pyrrolidin-2-yl]methoxy]-7-(8-chloro-1-naphthyl)-6,8-dihydro-SH-pyrido[3,4-d]pyrimidin-4-yl]-2-(cyanomethyl)piperazine-1-carboxylate (84 mg, 98% yield) as a white solid.

Step 4. To a solution of 2-trimethylsilylethyl (2S)-4-[2-[[(2S)-1-[3-[[(2S)-2-[3-(tert-butoxycarbonylamino)propyl]-5-(2,5-difluorophenyl)-2-phenyl-1,3,4-thiadiazole-3-carbonyl]-methyl-amino]propyl]pyrrolidin-2-yl]methoxy]-7-(8-chloro-1-naphthyl)-6,8-dihydro-SH-pyrido[3,4-d]pyrimidin-4-yl]-2-(cyanomethyl) piperazine-1-carboxylate (90 mg, 75.4 μmol, 1.0 equiv) in THF (1 mL) was added TBAF (1 M, 226.3 μL, 3.0 equiv). The mixture was stirred at 25° C. for 12 h. The residue was diluted with H₂O (10 mL) and extracted with DCM (10 mL×4), dried over Na₂SO₄, filtered and concentrated under reduced pressure to give compound tert-butyl N-[3-[(2S)-3-[3-[(2S)-2-[[7-(8-chloro-1-naphthyl)-4-[(3S)-3-(cyanomethyl)piperazin-1-yl]-6,8-dihydro-5H-pyridol[3,4-d]pyrimidin-2-yl]oxymethyl]pyrrolidin-1-yl]propyl-methyl-carbamoyl]-5-(2,5-difluorophenyl)-2-phenyl-1,3,4-thiadiazol-2-yl]propyl]carbamate (72 mg, 91% yield) as a white solid. LC-MS: MS (ES⁺): RT=0.935 min, m/z=1048.8 [M+H⁺];

Step 5. To a solution of tert-butyl N-[3-[(2S)-3-[3-[(2S)-2-[[7-(8-chloro-1-naphthyl)-4-[(3S)-3-(cyanomethyl)piperazin-1-yl]-6,8-dihydro-5H-pyrido [3,4-d]pyrimidin-2-yl]oxymethyl]pyrrolidin-1-yl]propyl-methyl-carbamoyl]-5-(2,5-difluorophenyl)-2-phenyl-1,3,4-thiadiazol-2-yl]propyl]carbamate (35 mg, 33.8 μmol, 1.0 equiv) and 2-fluoroprop-2-enoic acid (6.0 mg, 66.7 μmol, 2.0 equiv) in DMF (0.5 mL) was added DIEA (17.2 mg, 133.5 μmol, 23.5 μL, 4.0 equiv) and HATU (25.8 mg, 66.7 μmol, 2.0 equiv). The mixture was stirred at 25° C. for 0.5 h. The residue was purified by prep-HPLC (column: Phenomenex C18 75*30 mm*3 m; mobile phase: [water (FA)-ACN]; B%: 38%-68%, 7 min) to give compound tert-butyl N-[3-[(2S)-3-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]propyl-methyl-carbamoyl]-5-(2,5-difluorophenyl)-2-phenyl-1,3,4-thiadiazol-2-yl]propyl]carbamate (20 mg, 53% yield) as a yellow solid.

Step 6. To a solution of tert-butyl N-[3-[(2S)-3-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-SH-pyrido[3,4-d]pyrimidin-2-yl]oxymethyl]pyrrolidin-1-yl]propyl-methyl-carbamoyl]-5-(2,5-difluorophenyl)-2-phenyl-1,3,4-thiadiazol-2-yl]propyl]carbamate (20 mg, 17.8 μmol, 1.0 equiv) in DCM (0.5 mL) was added TFA (0.2 mL). The mixture was stirred at 25° C. for 0.5 h. The reaction mixture was concentrated under reduced pressure to remove solvent at 0° C. The residue was purified by prep-HPLC (column: Phenomenex C18 75*30 mm*3 m; mobile phase: [water (FA)-ACN]; B%: 20%-50%, 7 min) to give compound (2S)-2-(3-aminopropyl)-N-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-SH-pyrido[3,4-d]pyrimidin-2-yl]oxymethyl]pyrrolidin-1-yl]propyl]-5-(2,5-difluorophenyl)-N-methyl-2-phenyl-1,3,4-thiadiazole-3-carboxamide (9.5 mg, 49% yield, 97% purity, FA salt) as a yellow solid. ¹H NMR (400 MHz, CD₃OD): δ8.54 (s, 1H), 7.79-7.87 (m, 1H), 7.64-7.72 (m, 1H), 7.42-7.56 (m, 5H), 7.27-7.40 (m, 4H), 7.14-7.25 (m, 3H), 5.22-5.40 (m, 2H), 3.95-4.43 (m, 7H), 3.39-3.75 (m, 5H), 2.78-3.21 (m, 16H), 2.20-2.70 (m, 5H), 1.99-2.18 (m, 2H), 1.68-1.91 (m, 6H). LC-MS: MS (ES⁺): RT=2.13 min, m/z=1020.3 [M+H⁺]; LCMS method: 25.

### Example 82—Synthesis of Compound I-98

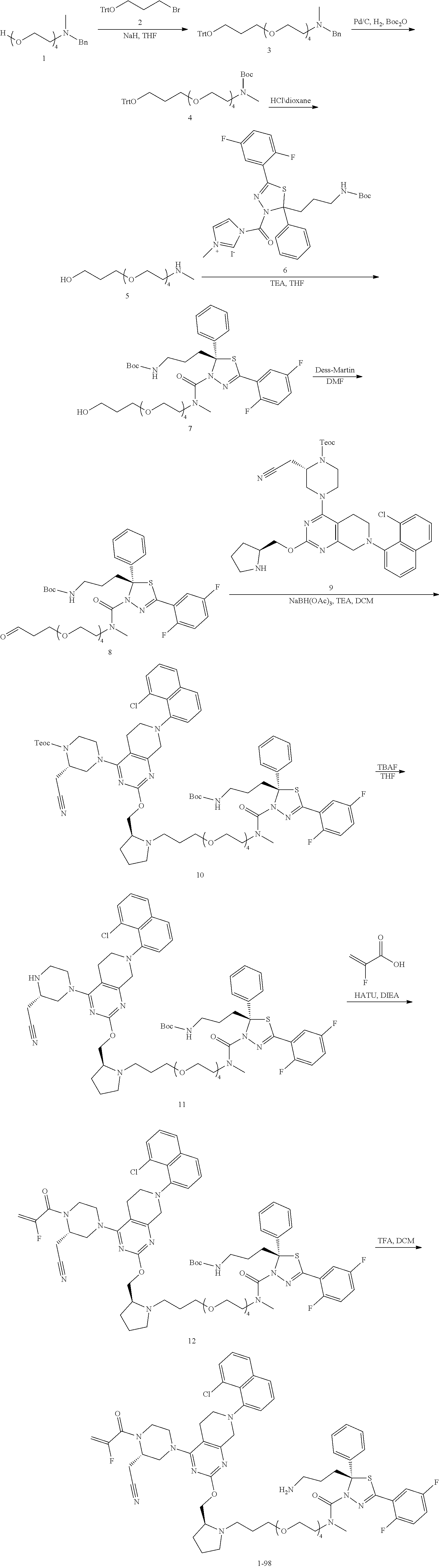

General Information: Compound 6 is a known compound from US2006/247178, 2006, A1.

Step 1. To a solution of 2-[2-[2-[2-[benzyl(methyl)amino]ethoxy]ethoxy]ethoxy]ethanol (3.2 g, 11 mmol, 1 equiv) in THF (5 mL) was added NaH (516 mg, 13 mmol, 60% purity, 1.2 equiv) at 0° C.,. The mixture was stirred at 20° C. for 0.5 h. Then [3-bromopropoxy(diphenyl)methyl]benzene (4.92 g, 13 mmol, 1.2 equiv) was added, The mixture was stirred at 20° C. for 11.5 h. The mixture was poured into sat.NH₄Cl solution (150 mL) and extracted with EtOAc (3×100 mL).The combined organic layers were washed with brine (50 mL), dried over Na₂SO₄, filtered and concentrated. The residue was purified by prep-HPLC (column: Phenomenex luna C18 150*40 mm*15 m; mobile phase: [water(FA)-ACN]; B%: 30%-60%, 10 min, column: Phenomenex luna C18 150*40 mm* 15 m; mobile phase: [water(FA)-ACN]; B%: 30%-60%, 10 min) to give N-benzyl-N-methyl-2-[2-[2-[2-(3-trityloxypropoxy)ethoxy]ethoxy]ethoxy]ethanamine (1.4 g, 22%yield). ¹H NMR (400 MHz, MeOD): δ7.42-7.20 (m, 20H), 3.65-3.50 (m, 18H), 3.27-3.22 (m, 2H), 2.70-2.65 (m, 2H), 2.29 (s, 3H), 1.85-1.81 (m, 2H).

Step 2. To a solution of N-benzyl-N-methyl-2-[2-[2-[2-(3-trityloxypropoxy) ethoxy]ethoxy]ethoxy]ethanamine (1.3 g, 2.17 mmol, leq) and tert-butoxycarbonyl tert-butyl carbonate (2.37 g, 11 mmol, 5 eq) in THF (20 mL) was added Pd/C (500 mg, 10% purity) under N₂ atmosphere. The mixture was stirred under H₂ (15 Psi) at 25° C. for 12 h. The mixture was filtered and concentrated. The residue was purified by prep-HPLC (column: Waters Xbridge C18 150*50 mm*10 m; mobile phase: [water(NH₄HCO₃)-ACN]; B%: 58%-88%, 11 min) to give tert-butyl N-methyl-N-[2-[2-[2-[2-(3-trityloxypropoxy)ethoxy]ethoxy]ethoxy]ethyl]carbamate (710 mg, 54% yield).

Step 3. To a solution of tert-butyl N-methyl-N-[2-[2-[2-[2-(3-trityloxypropoxy) ethoxy]ethoxy]ethoxy]ethyl]carbamate (325 mg, 535 μmol, 1 eq) in dioxane (1 mL) was added HCl/dioxane (4 M, 134 μL, 1 equiv). The mixture was stirred at 25° C. for 0.5 h and concentrated. The residue was used directly for next step.

Step 4. To a solution of 3-[2-[2-[2-[2-(methylamino)ethoxy]ethoxy]ethoxy]ethoxy]propan-1-ol (160 mg, 530 μmol, 1 equiv, HCl) in THF (2 mL) was added TEA (268 mg, 2.65 mmol, 5 eq) and tert-butyl N-[3-[5-(2,5-difluorophenyl)-3-(3-BLAH-3-methyl-imidazole-1-carbonyl)-2-phenyl-1,3,4-thiadiazol-2-yl]propyl]carbamate (426 mg, 636 μmol, 1.2 equiv). The mixture was stirred at 25° C. for 2 h. The residue was purified by prep-HPLC (column: Waters Xbridge 150*25 mm*5 m; mobile phase: [water(NH₄HCO₃)-ACN]; B%: 53%-83%, 10 min) to give tert-butyl (S)-(3-(5-(2,5-difluorophenyl)-3-((15-hydroxy-3,6,9,12-tetraoxapentadecyl)(methyl) carbamoyl)-2-phenyl-2,3-dihydro-1,3,4-thiadiazol-2-yl)propyl)carbamate (204 mg, 53%yield).

### ¹H NMR (400 MHz, MeOD): δ7.54-7.52 (m, 3H), 7.38-7.25 (m, 5H), 3.64-3.63 (m, 2H), 3.61-3.58 (m, 18H), 3.25-3.00 (m, 6H), 2.50-2.40 (m, 1H), 2.01-1.88 (m, 1H), 1.78-1.74 (m, 2H), 1.70-1.60 (m, 1H), 1.42 (s, 9H).

Step 5. To a solution of tert-butyl N-[3-[5-(2,5-difluorophenyl)-3-[2-[2-[2-[2-(3-hydroxypropoxy)ethoxy]ethoxy]ethoxy]ethyl-methylcarbamoyl]-2-phenyl-1,3,4-thiadiazol-2-yl]propyl]carbamate (200 mg, 276 μmol, 1 eq) in DMF (3 mL) was added Dess-Martin (293 mg, 690 μmol, 2.5 eq). The mixture was stirred at 25° C. for 0.5 h. The mixture was filtered and concentrated. The residue was used directly for next step.

Step 6. To a solution of tert-butyl N-[3-[5-(2,5-difluorophenyl)-3-[methyl-[2-[2-[2-[2-(3-oxopropoxy)ethoxy]ethoxy]ethoxy]ethyl]carbamoyl]-2-phenyl-1,3,4-thiadiazol-2-yl]propyl]carbamate (190 mg, 263 μmol, leq) and 2-trimethylsilylethyl (2S)-4-[7-(8-chloro-1-naphthyl)-2-[[(2S)-pyrrolidin-2-yl]methoxy]-6,8-dihydro-5H-pyrido[3,4-d]pyrimidin-4-yl]-2-(cyanomethyl)piperazine-1-carboxylate (204 mg, 263 μmol, 1.0 equiv, TFA salt) in DCM (4 mL) was added NaBH(OAc)3 (557 mg, 2.63 mmol, 10 equiv) and TEA (133 mg, 1.31 mmol, 5 eq). The mixture was stirred at 25° C. for 0.5 h. The mixture was filtered and concentrated. The residue was purified by prep-HPLC (column: Phenomenex luna C18 150*25 mm*10 μm; mobile phase: [water(FA)-ACN]; B%: 57%-87%, 10 min) to give 2-trimethylsilylethyl (2S)-4-[2-[[(2S)-1-[3-[2[2[2-[2-[[2-[3-(tert-butoxycarbonylamino)propyl]-5-(2,5-difluorophenyl)-2-phenyl-1,3,4-thiadiazole-3-carbonyl]-methyl-amino]ethoxy]ethoxy]ethoxy]ethoxy]propyl]pyrrolidin-2-yl]methoxy]-7-(8-chloro-1-naphthyl)-6,8-dihydro-5H-pyrido[3,4-d]pyrimidin-4-yl]-2-(cyanomethyl)piperazine-1-carboxylate (270 mg, 75% yield).

Step 7. To a solution of 2-trimethylsilylethyl (2S)-4-[2-[[(2S)-1-[3-[2-[2-[2-[2-[[2-[3-(tert-butoxycarbonylamino)propyl]-5-(2,5-difluorophenyl)-2-phenyl-1,3,4-thiadiazole-3-carbonyl]-methyl-amino]ethoxy]ethoxy]ethoxy]ethoxy]propyl]pyrrolidin-2-yl]methoxy]-7-(8-chloro-1-naphthyl)-6,8-dihydro-5H-pyrido[3,4-d]pyrimidin-4-yl]-2-(cyanomethyl)piperazine-1-carboxylate (130 mg, 95 μmol, 1 eq) in THF (1 mL) was added TBAF (1 M, 1.63 mL, 17 equiv). The mixture was stirred at 25° C. for 3 h. The mixture was diluted with water (30 mL) and extracted with EtOAc (30 mL*3). The combined organic layers were washed with brine (30 mL), dried over Na₂SO₄, filtered and concentrated to give tert-butyl N-[3-[3-[2-[2-[2-[2-[3-[(2S)-2-1[[7-(8-chloro-1-naphthyl)-4-[(3S)-3-(cyanomethyl)piperazin-1-yl]-6,8-dihydro-SH-pyrido[3,4-d]pyrimidin-2-yl]oxymethyl]pyrrolidin-1-yl]propoxy]ethoxy]ethoxy]ethoxy]ethyl-methylcarbamoyl]-5-(2,5-difluorophenyl)-2-phenyl-1,3,4-thiadiazol-2-yl]propyl]carbamate (107 mg, crude).

Step 8. To a solution of 2-fluoroprop-2-enoic acid (16 mg, 175 μmol, 2 eq) and tert-butyl N-[3-[3-[[2[2-[2-2-[3-[(2S)-2-[[7-(8-chloro-1-naphthyl-4-[(3S)-3-(cyanomethyl) piperazin-1-yl]-6,8-dihydro-SH-pyrido[3,4-d]pyrimidin-2-yl]oxymethyl]pyrrolidin-1-yl]propoxy]ethoxy]ethoxy]ethoxy]ethyl-methyl-carbamoyl]-5-(2,5-difluorophenyl)-2-phenyl-1,3,4-thiadiazol-2-yl]propyl]carbamate (107 mg, 87 μmol, 1 equiv) in DMF (1 mL) was added HATU (40 mg, 105 μmol, 1.2 eq) and DIEA(34 mg, 262 μmol, 3 eq). The mixture was stirred at 25° C. for 1 h. The mixture was filtered and concentrated. The residue was purified by prep-HPLC (column: Phenomenex luna C18 250*50 mm*15 m; mobile phase: [water(FA)-ACN]; B%: 47%-77%, 10 min) to give tert-butyl N-[3-[3-[2-[2-[2-[2-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]propoxy]ethoxy]ethoxy]ethoxy]ethyl-methyl-carbamoyl]-5-(2,5-difluorophenyl)-2-phenyl-1,3,4-thiadiazol-2-yl]propyl]carbamate (40 mg, 35% yield).

Step 9. To a solution of tert-butyl N-[3-[3-[2-[2-[2-[2-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]propoxy]ethoxy]ethoxy]ethoxy]ethyl-methyl-carbamoyl]-5-(2,5-difluorophenyl)-2-phenyl-1,3,4-thiadiazol-2-yl]propyl]carbamate (30 mg, 23 μmol, 1 equiv) in DCM (3 mL) was added TFA (0.3 mL). The mixture was stirred at 25° C. for 0.5 h and concentrated. The residue was purified by prep-HPLC (column: Phenomenex luna C18 250*50 mm*15 m; mobile phase: [water(FA)-ACN]; B%: 20%-50%, 10 min, column: Phenomenex luna C18 250*50 mm*15 m; mobile phase: [water(FA)-ACN]; B%: 13%-43%, 10 min) to 2-(3-aminopropyl)-N-[2-[2-[2-[2-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]propoxy]ethoxy]ethoxy]ethoxy]ethyl]-5-(2,5-difluorophenyl)-N-methyl-2-phenyl-1,3,4-thiadiazole-3-carboxamide (3.04 mg, 9.52% yield). ¹H NMR (400 MHz, MeOD): δ8.61-8.47 (m, 2H), 7.82 (d, J=7.6 Hz, 1H), 7.70 (d, J=7.6 Hz, 1H), 7.53-7.49 (m, 5H), 7.40-7.25 (m, 6H), 5.38-5.25 (m, 2H), 4.50-4.40 (m, 2H), 4.20-4.10 (m, 4H), 3.70-3.40 (m, 24H), 3.30-3.25 (m, 2H), 3.22-3.00 (m, 1OH), 3.28-3.25 (m, 5H), 2.20-2.10 (m, 2H), 2.00-1.75 (m, 6H). LC-MS: MS (ES′): RT=2.03 min, m/z=1196.4 [M+H⁺]; LCMS method: 25.

### Example 83—Synthesis of Compound I-99

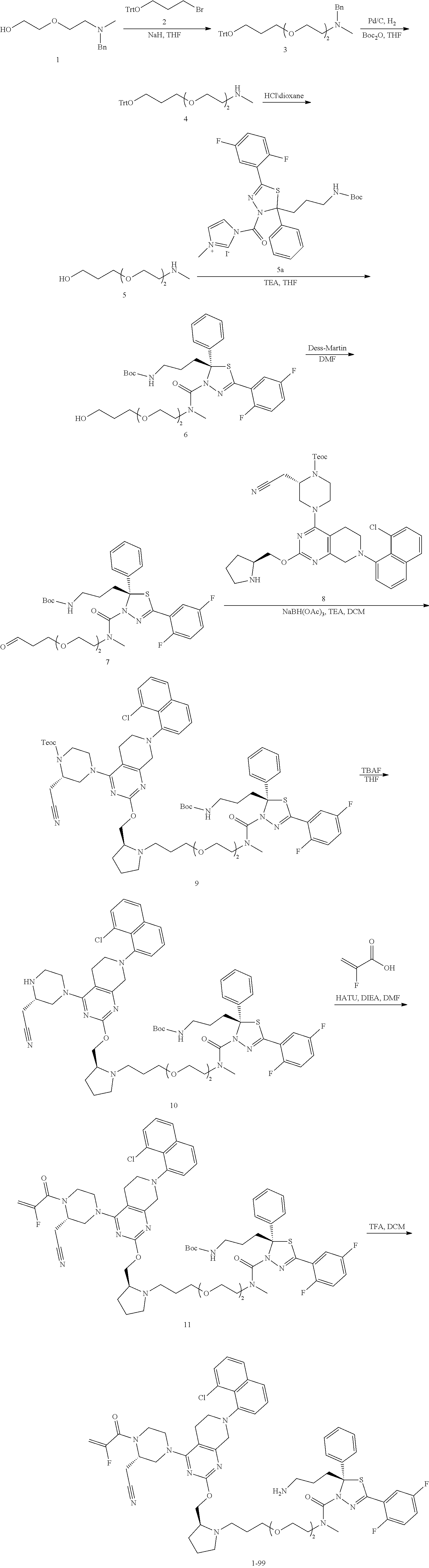

General Information: Compound 1 is a known compound from WO2016/118666, 2016, A1. Compound 2 is a known compound from US2008/96903, 2008, A1 Compound 5a is a known compound from US2006/247178, 2006, A1.

Step 1. To a solution of [3-bromopropoxy(diphenyl)methyl]benzene (8.75 g, 23 mmol, 1.2 equiv) in THF (40 mL) was added NaH (994 mg, 24.9 mmol, 60% purity, 1.3 equiv) at 0° C., The mixture was stirred at 20° C. for 0.5 h. Then 2-[2-[benzyl(methyl)amino]ethoxy]ethanol (4g, 19.1 mmol, 1 equiv) was added, The mixture was stirred at 20° C. for 11.5 h. The mixture was poured into sat.NH4Cl solution (150 mL) and extracted with EtOAc (3×200 mL), the combined organic layers were washed with brine (100 mL), dried over Na₂SO₄, filtered and concentrated. The residue was purified by prep-HPLC (column: Phenomenex luna C18 150*40 mm* 15 m; mobile phase: [water(FA)-ACN]; B%: 30%-60%, 10 min, column: Phenomenex luna C18 150*40 mm*15 m; mobile phase: [water(FA)-ACN]; B%: 30%-60%, 10 min) to give N-benzyl-N-methyl-2-[2-(3-trityloxypropoxy)ethoxy]ethanamine (3.2 g, 33% yield). ¹H NMR (400 MHz, MeOD): δ7.42-7.39 (m, 6H), 7.28-7.23 (m, 14H), 3.62-3.54 (m, 10H), 3.15-3.10 (m, 2H), 2.68-2.60 (m, 2H), 2.27 (s, 3H), 1.85-1.81 (m, 2H).

Step 2. To a solution of tert-butoxycarbonyl tert-butyl carbonate (6.85 g, 32 mmol, 5 equiv) and N-benzyl-N-methyl-2-[2-(3-trityloxypropoxy)ethoxy]ethanamine (3.2 g, 6.3 mmol, 1 equiv) in THF (1 mL) was added Pd/C (800 mg, 10% purity) under N₂. The mixture was stirred under H₂ (15 Psi) at 25° C. for 12 h. The mixture was filtered and concentrated. The residue was purified by prep-HPLC (column: Waters Xbridge C18 150*50 mm*10 m; mobile phase: [water(NH₄HCO₃)-ACN]11 min) to give tert-butyl N-methyl-N-[2-[2-(3-trityloxypropoxy) ethoxy]ethyl]carbamate (2.1 g, 64% yield). ¹H NMR (400 MHz, MeOD): δ7.42-7.39 (m, 6H), 7.28-7.23 (m, 9H), 3.62-3.54 (m, 8H), 3.35-3.16 (m, 2H), 2.85 (s, 2H), 1.85-1.81 (m, 2H), 1.44 (s, 9H).

Step 3. A solution of tert-butyl N-methyl-N-[2-[2-(3-trityloxypropoxy) ethoxy]ethyl]carbamate (450 mg, 866 μmol, 1 equiv) in HCl/dioxane (4 M, 4.50 mL) was stirred at 25° C. for 0.5 h. The mixture was concentrated. The residue was used directly for next step.

Step 4. To a solution of tert-butyl N-[3-[5-(2,5-difluorophenyl)-3-(3—N-3-methyl-imidazole-1-carbonyl)-2-phenyl-1,3,4-thiadiazol-2-yl]propyl]carbamate (563.94 mg, 842.30 μmol, 1 equiv) in THF (1 mL) was added TEA (426 mg, 4.2 mmol, 5 equiv) and 3-[2-[2-(methylamino)ethoxy]ethoxy]propan-1-ol (180 mg, 842 μmol, 1 equiv, HCl).The mixture was stirred at 25° C. for 2 h. The mixture was concentrated. The residue was purified by prep-HPLC (column: Waters Xbridge 150*25 mm*5 m; mobile phase: [water(NH₄HCO₃)-ACN]; B%: 54%-84%, 10 min) to give tert-butyl N-[3-[5-(2,5-difluorophenyl)-3-[2-[2-(3-hydroxypropoxy) ethoxy]ethyl-methyl-carbamoyl]-2-phenyl-1,3,4-thiadiazol-2-yl]propyl]carbamate (230 mg, 43% yield).

Step 5. To a solution of tert-butyl N-[3-[5-(2,5-difluorophenyl)-3-[2-[2-(3-hydroxypropoxy)ethoxy]ethyl-methyl-carbamoyl]-2-phenyl-1,3,4-thiadiazol-2-yl]propyl]carbamate (224 mg, 352 μmol, 1 equiv) in DMF (2 mL) was added Dess-Martin (448 mg, 1.06 mmol, 3 equiv). The mixture was stirred at 25° C. for 0.5 h. The mixture was concentrated. The residue was used directly for next step.

Step 6. To a solution of tert-butyl N-[3-[5-(2,5-difluorophenyl)-3-[methyl-[2-[2-(3-oxopropoxy)ethoxy]ethyl]carbamoyl]-2-phenyl-1,3,4-thiadiazol-2-yl]propyl]carbamate (223 mg, 351 μmol, 1 equiv) and 2-trimethylsilylethyl (2S)-4-[7-(8-chloro-1-naphthyl)-2-[[(2S)-pyrrolidin-2-yl]methoxy]-6,8-dihydro-5H-pyrido[3,4-d]pyrimidin-4-yl]-2-(cyanomethyl)piperazine-1-carboxylate (273 mg, 351 μmol, 1 equiv, TFA salt) in DCM (5 mL) was added NaBH(OAc)₃ (745 mg, 3.5 mmol, 10 equiv) and TEA (355 mg, 3.51 mmol, 489 L, 10 equiv). The mixture was stirred at 25° C. for 0.5 h and concentrated. The residue was purified by prep-HPLC (column: Phenomenex luna C18 250*50 mm*15 m; mobile phase: [water(FA)-ACN]; B%: 56%-86%, 10 min) to give 2-trimethylsilylethyl (2S)-4-[2-[[(2S)-1-[3-[2-[2-[[2-[3-(tert-butoxycarbonylamino) propyl]-5-(2,5-difluorophenyl)-2-phenyl-1,3,4-thiadiazole-3-carbonyl]-methyl-amino]ethoxy]ethoxy]propyl]pyrrolidin-2-yl]methoxy]-7-(8-chloro-1-naphthyl)-6,8-dihydro-SH-pyrido[3,4-d]pyrimidin-4-yl]-2-(cyanomethyl)piperazine-1-carboxylate (310 mg, 69% yield).

Step 7. To a solution of 2-trimethylsilylethyl (2S)-4-[2-[[(2S)-1-[3-[2-[2-[[2-[3-(tert-butoxycarbonylamino)propyl]-5-(2,5-difluorophenyl)-2-phenyl-1,3,4-thiadiazole-3-carbonyl]-methyl-amino]ethoxy]ethoxy]propyl]pyrrolidin-2-yl]methoxy]-7-(8-chloro-1-naphthyl)-6,8-dihydro-SH-pyrido[3,4-d]pyrimidin-4-yl]-2-(cyanomethyl)piperazine-1-carboxylate (270 mg, 211 μmol, 1 equiv) and tetrabutyl ammonium fluoride trihydrate (1 M, 6.7 mL, 32.03 equiv) in THF (1 mL). The mixture was stirred at 25° C. for 12 h. The mixture was diluted with water (10 mL) and extracted with DCM (30 mL×3). The combined organic layers were washed with brine (30 mL), dried over Na₂SO₄, filtered and concentrated. The residue was used directly for next step.

Step 8. To a solution of tert-butyl N-[3-[3-[2-[2-[3-[(2S)-2-[[7-(8-chloro-1-naphthyl)-4-[(3S)-3-(cyanomethyl)piperazin-1-yl]-6,8-dihydro-SH-pyrido[3,4-d]pyrimidin-2-yl]oxymethyl]pyrrolidin-1-yl]propoxy]ethoxy]ethyl-methyl-carbamoyl]-5-(2,5-difluorophenyl)-2-phenyl-1,3,4-thiadiazol-2-yl]propyl]carbamate (190 mg, 167 μmol, 1 equiv) and 2-fluoroprop-2-enoic acid (30 mg, 334 μmol, 2 equiv) in DMF (0.5 mL) was added HATU (76 mg, 200 μmol, 1.2 eq) and DIEA (65 mg, 501 μmol, 3 equiv). The mixture was stirred at 25° C. for 1 h and concentrated. The residue was purified by prep-HPLC (column: Phenomenex luna C18 250*50 mm*15 m; mobile phase: [water(FA)-ACN]; B%: 44%-74%, 10 min) to give tert-butyl N-[3-[3-[2-[2-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]propoxy]ethoxy]ethyl-methyl-carbamoyl]-5-(2,5-difluorophenyl)-2-phenyl-1,3,4-thiadiazol-2-yl]propyl]carbamate (50 mg, 25% yield).

Step 9. To a solution of tert-butyl N-[3-[3-[2-[2-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]propoxy]ethoxy]ethyl-methyl-carbamoyl]-5-(2,5-difluorophenyl)-2-phenyl-1,3,4-thiadiazol-2-yl]propyl]carbamate (30 mg, 25 μmol, 1 equiv) in DCM (2 mL) was added TFA (200.00 μL). The mixture was stirred at 25° C. for 0.5 h and concentrated. The residue was purified by prep-HPLC (column: Phenomenex luna C18 250*50 mm*15 m; mobile phase: [water(FA)-ACN]; B%: 24%-54%, 10 min) to give 2-(3-aminopropyl)-N-[2-[2-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]propoxy]ethoxy]ethyl]-5-(2,5-difluorophenyl)-N-methyl-2-phenyl-1,3,4-thiadiazole-3-carboxamide (2.5 mg, 7% yield). ¹H NMR (400 MHz, MeOD): δ8.54 (s, 1H), 7.82-7.80 (m, 1H), 7.55-7.52 (m, 1H), 7.51-7.45 (m, 5H), 7.40-7.22 (m, 7H), 5.34-5.25 (m, 2H), 4.40-4.15 (m, 2H), 3.68-3.49 (m, 15H), 3.25-3.00 (m, 14H), 2.50-2.01 (m, 5H), 1.85-1.55 (m, 6H), 1.35-1.30 (m, 5H). LC-MS: MS (ES⁺): RT=2.131 min, m/z=1108.4 [M+H⁺]; LCMS method: 25.

### Example 84—Synthesis of Compound I-100

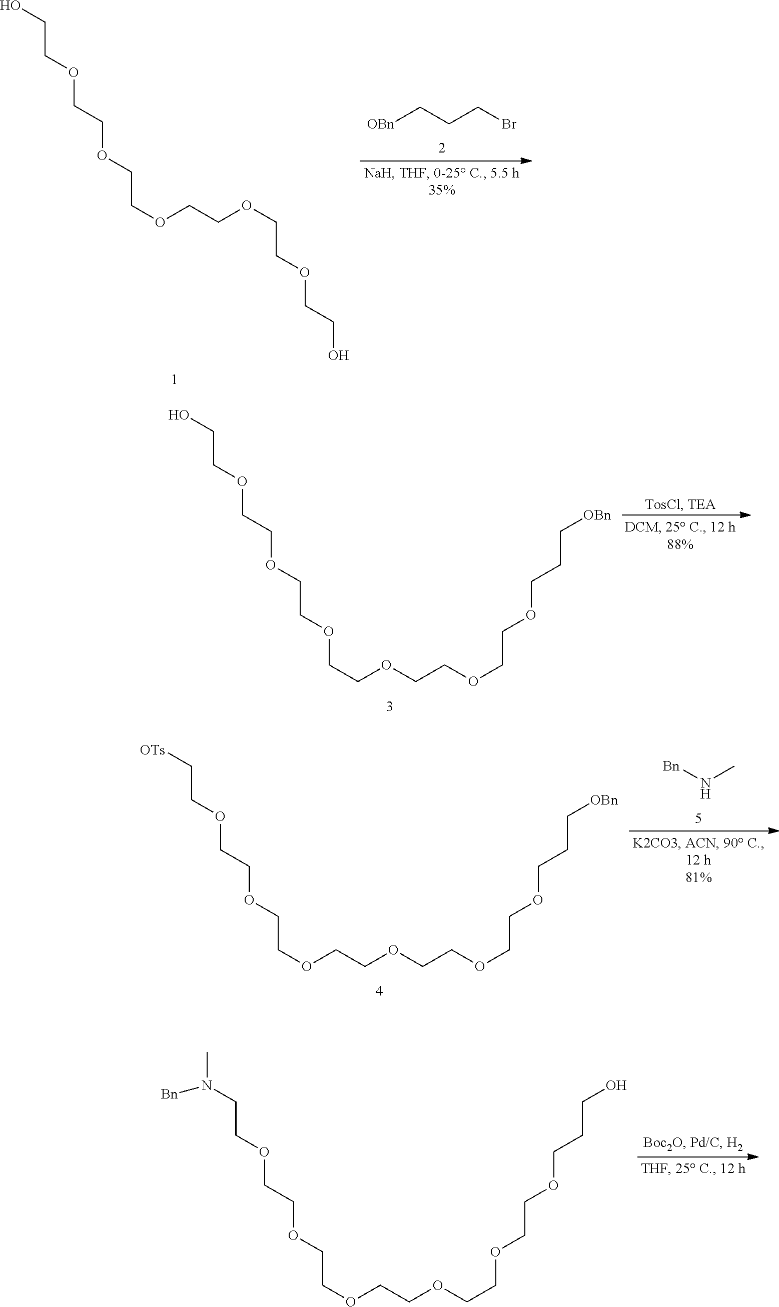

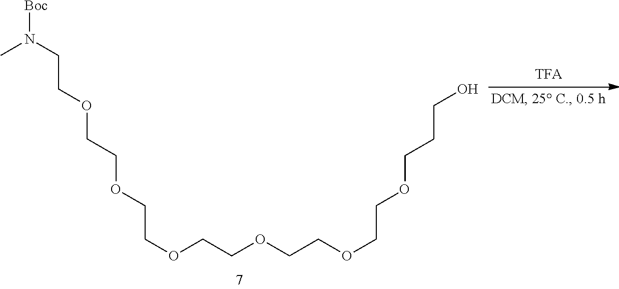 [SMILES: CN(CCOCCOCCOCCOCCOCCOCCCO)C(=O)OC(C)(C)C]

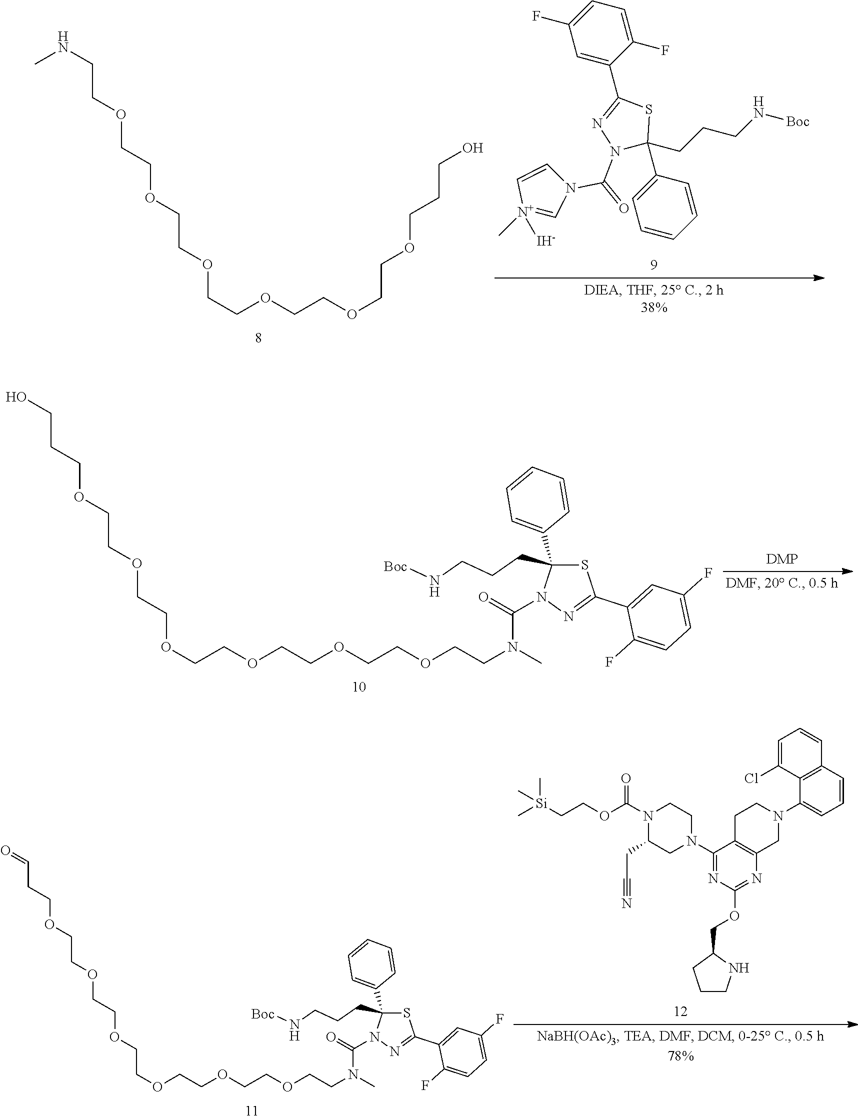

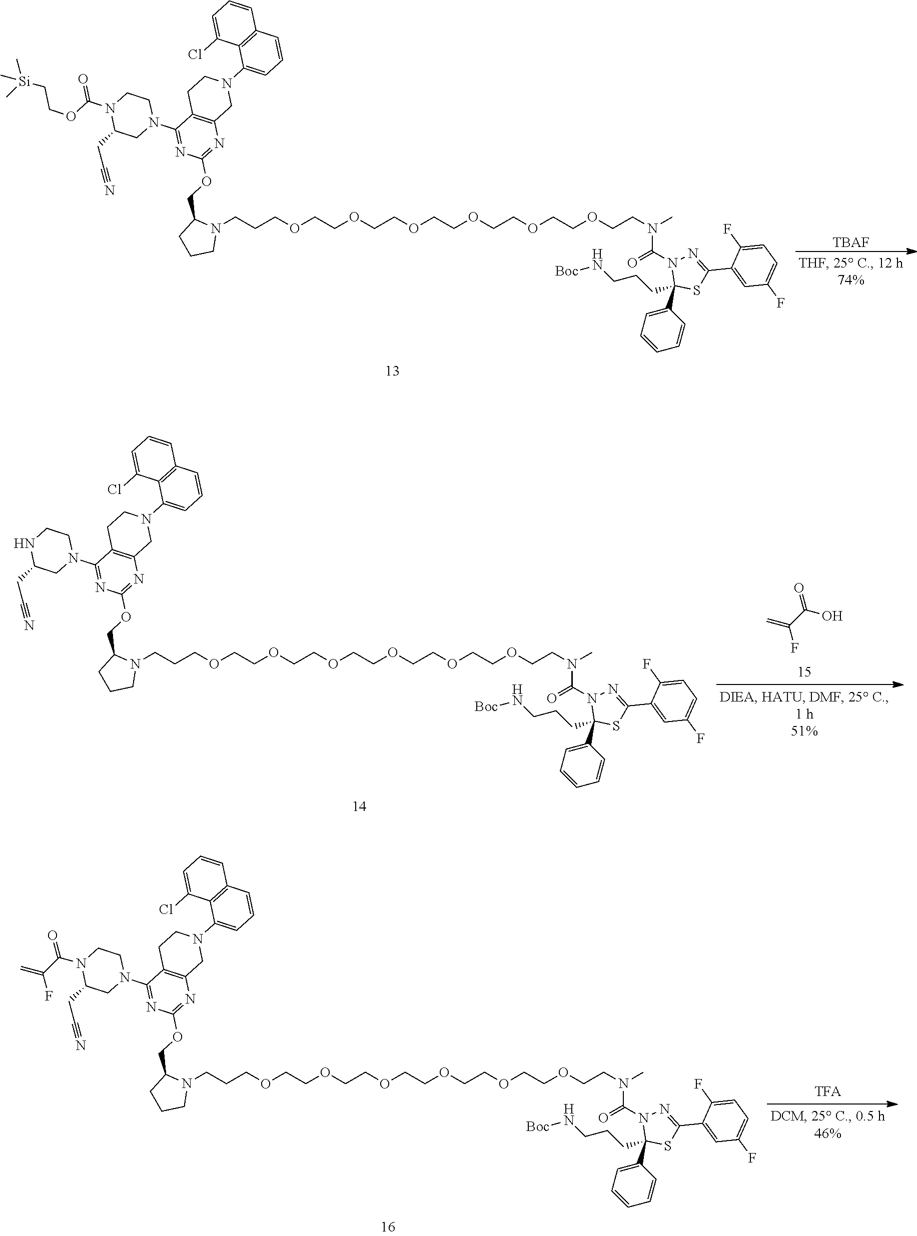

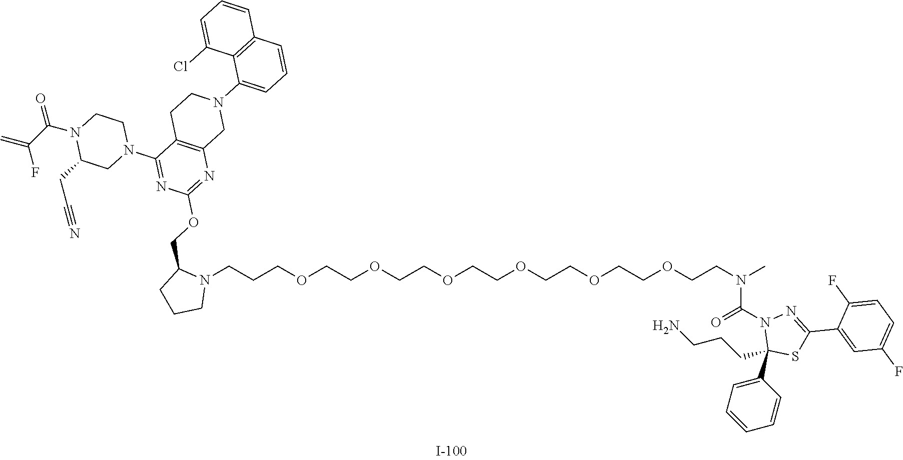 [SMILES: C=C(F)C(=O)N1CCN(c2nc(OC[C@@H]3CCCN3CCCOCCOCCOCCOCCOCCOCCN(C)C(=O)N3N=C(c4cc(F)ccc4F)S[C@@]3(CCCN)c3ccccc3)nc3c2CCN(c2cccc4cccc(Cl)c24)C3)C[C@@H]1CC#N]

Step 1. To a solution of 2-[2-[2-[2-[2-(2-hydroxyethoxy)ethoxy]ethoxy]ethoxy]ethoxy]ethanol (5.55 g, 19.6 mmol, 1.5 equiv) in THF (50 mL) was added NaH (785.5 mg, 19.64 mmol, 60% purity, 1.5 equiv) at 0° C., after stirring for 0.5 h, 3-bromopropoxymethylbenzene (3.00 g, 13.0 mmol, 2.31 mL, 1.0 equiv) was added and then it was stirred at 25° C. for 5 h. This reaction was quenched by aq. NH₄Cl (5 mL) and concentrated to afford crude product. The residue was purified by prep-HPLC (column: Kromasil Eternity XT 250*80 mm*10 m; mobile phase: [water(NH₄HCO₃)-ACN]; B%: 28ACN %-58ACN %, 14 min) to afford 2-[2-[2-[2-[2-[2-(3-benzyl oxypropoxy)ethoxy]ethoxy]ethoxy]ethoxy]ethoxy]ethanol (2.00 g, 4.65 mmol, 35% yield) as a yellow oil.

Step 2. To a solution of 2-[2-[2-[2-[2-[2-(3-benzyloxypropoxy)ethoxy]ethoxy]ethoxy]ethoxy]ethoxy]ethanol (2.00 g, 4.65 mmol, 1.0 equiv) in DCM (10 mL) was added TosCl (2.21 g, 11.6 mmol, 2.5 equiv) and TEA (1.41 g, 13.9 mmol, 1.94 mL, 3.0 equiv). The mixture was stirred at 25° C. for 12 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by prep-HPLC (column: Waters Xbridge C18 150*50 mm*10 m; mobile phase: [water(NH4HCO₃)-ACN]; B%: 43%-73%, 11 min) to afford 2-[2-[2-[2-[2-[2-(3-benzyloxypropoxy) ethoxy]ethoxy]ethoxy]ethoxy]ethoxy]ethyl 4-methylbenzenesulfonate (2.40 g, 4.10 mmol, 88% yield) as a yellow oil.

Step 3. To a solution of 2-[2-[2-[2-[2-[2-(3-benzyloxypropoxy)ethoxy]ethoxy]ethoxy]ethoxy]ethoxy]ethyl 4-methylbenzenesulfonate (2.3 g, 3.9 mmol, 1.0 equiv) and N-methyl-1-phenyl-methanamine (571 mg, 4.72 mmol, 609 μL, 1.2 equiv) inACN (30 mL) was added K₂CO₃ (1.09 g, 7.87 mmol, 2.0 equiv). The mixture was stirred at 90° C. for 12 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by prep-HPLC (column: Waters Xbridge C18 150*50 mm*10 m; mobile phase: [water(NH₄HCO₃)-ACN]; B%: 45%-75%, 11 min) to afford N-benzyl-2-[2-[2-[2-[2-[2-(3-benzyloxypropoxy)ethoxy]ethoxy]ethoxy]ethoxy]ethoxy]-N-methyl-ethanamine (1.7 g, 3.1 mmol, 81% yield) as a yellow oil.

Step 4. To a solution of N-benzyl-2-[2-[2-[2-[2-[2-(3-benzyloxypropoxy) ethoxy]ethoxy]ethoxy]ethoxy]ethoxy]-N-methyl-ethanamine (1.70 g, 3.19 mmol, 1.0 equiv) in THF (10 mL) was added Pd/C (200 mg, 10% purity,) and Boc₂0 (834 mg, 3.82 mmol, 878 μL, 1.2 equiv) under N₂ atmosphere. The suspension was degassed and purged with H₂ for 3 times. The mixture was stirred under H₂ (15 psi) at 25° C. for 12 h. After filtered, the filtrate was concentrated to give tert-butyl N-[2-[2-[2-[2-[2-[2-(3-hydroxypropoxy)ethoxy]ethoxy]ethoxy]ethoxy]ethoxy]ethyl]-N-methyl-carbamate (1.6 g, crude) as a colorless oil and used for next step directly.

Step 5. To a solution of tert-butyl N-[2-[2-[2-[2-[2-[2-(3-hydroxypropoxy)ethoxy]ethoxy]ethoxy]ethoxy]ethoxy]ethyl]-N-methyl-carbamate (210 mg, 463 μmol, 1.0 equiv) in DCM (1.5 mL) was added TFA (0.5 mL). The mixture was stirred at 25° C. for 0.5 h. The reaction mixture was concentrated to afford 3-[[2[2-[2-[2-[2-(methylamino)ethoxy]ethoxy]ethoxy]ethoxy]ethoxy]ethoxy]propan-1-ol (210 mg, 449 μmol, crude, TFA salt) as a yellow oil and it was used by next step directly.

Step 6. To a solution of 3-[2-[2-[2-[2-[2-[2-(methylamino)ethoxy]ethoxy]ethoxy]ethoxy]ethoxy]ethoxy]propan-1-ol (210 mg, 449 μmol, 1.0 equiv, TFA salt) and DIEA (348 mg, 2.70 mmol, 469 μL, 6.0 equiv) in THF (4 mL) was added tert-butyl N-[3-[5-(2,5-difluorophenyl)-3-(3-BLAH-3-methyl-imidazole-1-carbonyl)-2-phenyl-1,3,4-thiadiazol-2-yl]propyl]carbamate (300 mg, 449 μmol, 1.0 equiv), and then it was stirred at 25° C. for 2 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by prep-HPLC (column: Waters Xbridge C18 150*50 mm*10 m; mobile phase: [water(NH4HCO₃)-ACN]; B%: 41%-71%, 11 min) and (column: Waters Xbridge C18 150*50 mm*10 m; mobile phase: [water(NH₄HCO₃)-ACN]; B%: 47%-77%, 11 min) to afford tert-butyl N-[3-[(2S)-5-(2,5-difluorophenyl)-3-[2-[2-[2-[2-[2-[2-(3-hydroxypropoxy)ethoxy]ethoxy]ethoxy]ethoxy]ethoxy]ethyl-methyl-carbamoyl]-2-phenyl-1,3,4-thiadiazol-2-yl]propyl]carbamate (140 mg, 172 μmol, 38% yield) as a yellow oil.

Step 7. To a solution of tert-butyl N-[3-[(2S)-5-(2,5-difluorophenyl)-3-[2-[2-[2-[2-[2-[2-(3-hydroxypropoxy)ethoxy]ethoxy]ethoxy]ethoxy]ethoxy]ethyl-methyl-carbamoyl]-2-phenyl-1,3,4-thiadiazol-2-yl]propyl]carbamate (130 mg, 159 μmol, 1.0 equiv) in DMF (0.5 mL) was added DMP (271 mg, 639 μmol, 198 μL, 4.0 equiv). The mixture was stirred at 20° C. for 0.5 h. tert-butyl N-[3-[(2S)-5-(2,5-difluorophenyl)-3-[methyl-[2-[2-[2-[2-[2-[2-(3-oxopropoxy)ethoxy]ethoxy]ethoxy]ethoxy]ethoxy]ethyl]carbamoyl]-2-phenyl-1,3,4-thiadiazol-2-yl]propyl]carbamate (129 mg, 159 μmol) was in yellow liquid and it was used by next step directly.

Step 8. To a solution of 2-trimethylsilylethyl (2S)-4-[7-(8-chloro-1-naphthyl)-2-[[(2S)-pyrrolidin-2-yl]methoxy]-6,8-dihydro-5H-pyrido[3,4-d]pyrimidin-4-yl]-2-(cyanomethyl) piperazine-1-carboxylate (123 mg, 159 μmol, 1.0 equiv, TFA salt) in DCM (3 mL) was added TEA (160 mg, 1.59 mmol, 221 μL, 10.0 equiv), AfterpH was adjusted to 7-8, NaBH(OAc)₃ (337 mg, 1.59 mmol, 10.0 equiv) was added. Then tert-butyl N-[3-[(2S)-5-(2,5-difluorophenyl)-3-[methyl-[2-[2-[2-[2-[2-[2-(3-oxopropoxy)ethoxy]ethoxy]ethoxy]ethoxy]ethoxy]ethyl]carbamoyl]-2-phenyl-1,3,4-thiadiazol-2-yl]propyl]carbamate (129 mg, 159 μmol, 1.0 equiv) in DMF (1 mL) was dropwise added at 0° C. The reaction mixture was stirred at 20° C. for 0.5 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by prep-HPLC (column: Phenomenex luna C18 250*50 mm*15 m; mobile phase: [water(FA)-ACN]; B%: 56%-86%, 10 min) to afford 2-trimethylsilylethyl (2S)-4-[2-[[(2S)-1-[3-[2-[2-[2-[2-[2-[2-[[(2S)-2-[3-(tert-butoxycarbonyl-amino) propyl]-5-(2,5-difluorophenyl)-2-phenyl-1,3,4-thiadiazole-3-carbonyl]-methyl-amino]ethoxy]ethoxy]ethoxy]ethoxy]ethoxy]ethoxy]propyl]pyrrolidin-2-yl]methoxy]-7-(8-chloro-1-naphthyl)-6,8-dihydro-5H-pyrido[3,4-d]pyrimidin-4-yl]-2-(cyanomethyl)piperazine-1-carboxylate (180 mg, 123 μmol, 77% yield) as a yellow oil.

Step 9. To a solution of 2-trimethylsilylethyl (2S)-4-[2-[[(2S)-1-[3-[2-[2-[2-[2-[2-[2-[[(2S)-2-[3-(tert-butoxycarbonylamino)propyl]-5-(2,5-difluorophenyl)-2-phenyl-1,3,4-thiadiazole-3-carbonyl]-methylamino]ethoxy]ethoxy]ethoxy]ethoxy]ethoxy]ethoxy]propyl]pyrrolidin-2-yl]methoxy]-7-(8-chloro-1-naphthyl)-6,8-dihydro-5H-pyrido[3,4-d]pyrimidin-4-yl]-2-(cyanomethyl)piperazine-1-carboxylate (150 mg, 102 μmol, 1.0 equiv) in THF (1 mL) was added TBAF (1 M, 1.03 mL, 10.0 equiv). The mixture was stirred at 25° C. for 12 h. To the reaction mixture was added water (50 mL) and the mixture was extracted with EtOAc (50 mL). The combined organic phase was washed with brine (50 mL×3), dried over anhydrous Na₂SO₄, filtered and concentrated to afford tert-butyl N-[3-[(2S)-3-[2-[2-[2-[2-[2-[2-[3-[(2S)-2-[[7-(8-chloro-1-naphthyl)-4-[(3S)-3-(cyanomethyl)piperazin-1-yl]-6,8-dihydro-5H-pyrido[3,4-d]pyrimidin-2-yl]oxymethyl]pyrrolidin-1-yl]propoxy]ethoxy]ethoxy]ethoxy]ethoxy]ethoxy]ethyl-methyl-carbamoyl]-5-(2,5-difluorophenyl)-2-phenyl-1,3,4-thiadiazol-2-yl]propyl]carbamate (100 mg, 76.1 μmol, 74% yield) as a yellow oil and it was used by next step directly.

Step 10. To a solution of tert-butyl N-[3-[(2S)-3-[2-[2-[2-[2-[2-[2-[3-[(2S)-2-[[7-(8-chloro-1-naphthyl)-4-[(3S)-3-(cyanomethyl)piperazin-1-yl]-6,8-dihydro-5H-pyrido[3,4-d]pyrimidin-2-yl]oxymethyl]pyrrolidin-1-yl]propoxy]ethoxy]ethoxy]ethoxy]ethoxy]ethoxy]ethyl-methyl-carbamoyl]-5-(2,5-difluorophenyl)-2-phenyl-1,3,4-thiadiazol-2-yl]propyl]carbamate (70 mg, 53 μmol, 1.0 equiv) and 2-fluoroprop-2-enoic acid (48.01 mg, 533.1 μmol, 10.0 equiv) in DMF (2 mL) was added DIEA (34.45 mg, 266.5 μmol, 46.43 μL, 5.0 equiv) and HATU (40.54 mg, 106.6 μmol, 2.0 equiv). The mixture was stirred at 25° C. for 1 h. The reaction mixture was diluted with 20 mL water, extracted with EA (2×10 mL), dried over anhydrous Na₂SO₄, filtered and concentrated to afford tert-butyl N-[3-[(2S)-3-[2-[2-[2-[2-[2-[2-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]propoxy]ethoxy]ethoxy]ethoxy]ethoxy]ethoxy]ethyl-methyl-carbamoyl]-5-(2,5-difluorophenyl)-2-phenyl-1,3,4-thiadiazol-2-yl]propyl]carbamate (38 mg, 27 μmol, 51% yield) as a yellow solid and it was used by next step directly.

Step 11. To a solution of tert-butyl N-[3-[(2S)-3-[2-[2-[2-[2-[2-[2-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-SH-pyrido[3,4-d]pyrimidin-2-yl]oxymethyl]pyrrolidin-1-yl]propoxy]ethoxy]ethoxy]ethoxy]ethoxy]ethoxy]ethyl-methyl-carbamoyl]-5-(2,5-difluorophenyl)-2-phenyl-1,3,4-thiadiazol-2-yl]propyl]carbamate (35 mg, 25 μmol, 1.0 equiv) in DCM (1 mL) was added TFA (0.2 mL). The mixture was stirred at 25° C. for 0.5 h. The reaction mixture was concentrated in vacuum. The residue was purified by prep-HPLC (column: Phenomenex C18 75*30 mm*3 m; mobile phase: [water(FA)-ACN]; B%: 25%-55%, 7 min) to give (2S)-2-(3-aminopropyl)-N-[2-[22[2-[2-[2-[2-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]propoxy]ethoxy]ethoxy]ethoxy]ethoxy]ethoxy]ethyl]-5-(2,5-difluorophenyl)-N-methyl-2-phenyl-1,3,4-thiadiazole-3-carboxamide (15 mg, 11 μmol, 46% yield, 99.9% purity) as a yellow solid. ¹H NMR (400 MHz, CD₃OD) δ8.55 (s, 1H), 7.86-7.74 (m, 2H), 7.68 (m, 1H), 7.55-7.45 (m, 4H), 7.40-7.26 (m, 5H), 7.16-7.02 (m, 2H), 5.41-5.23 (m, 2H), 4.42-4.02 (m, 6H), 3.83-3.34 (m, 31H), 3.25-2.85 (m, 14H), 2.76-2.61 (m, 1H), 2.57-2.38 (m, 2H), 2.36-2.26 (m, 1H), 2.19-1.98 (m, 2H), 1.92-1.65 (m, 6H). LC-MS: MS (ES⁺): RT=2.253 min, m/z=642.8 [M/2+H⁺]; LCMS Method: 25.

### Example 85—Synthesis of Compound I-101

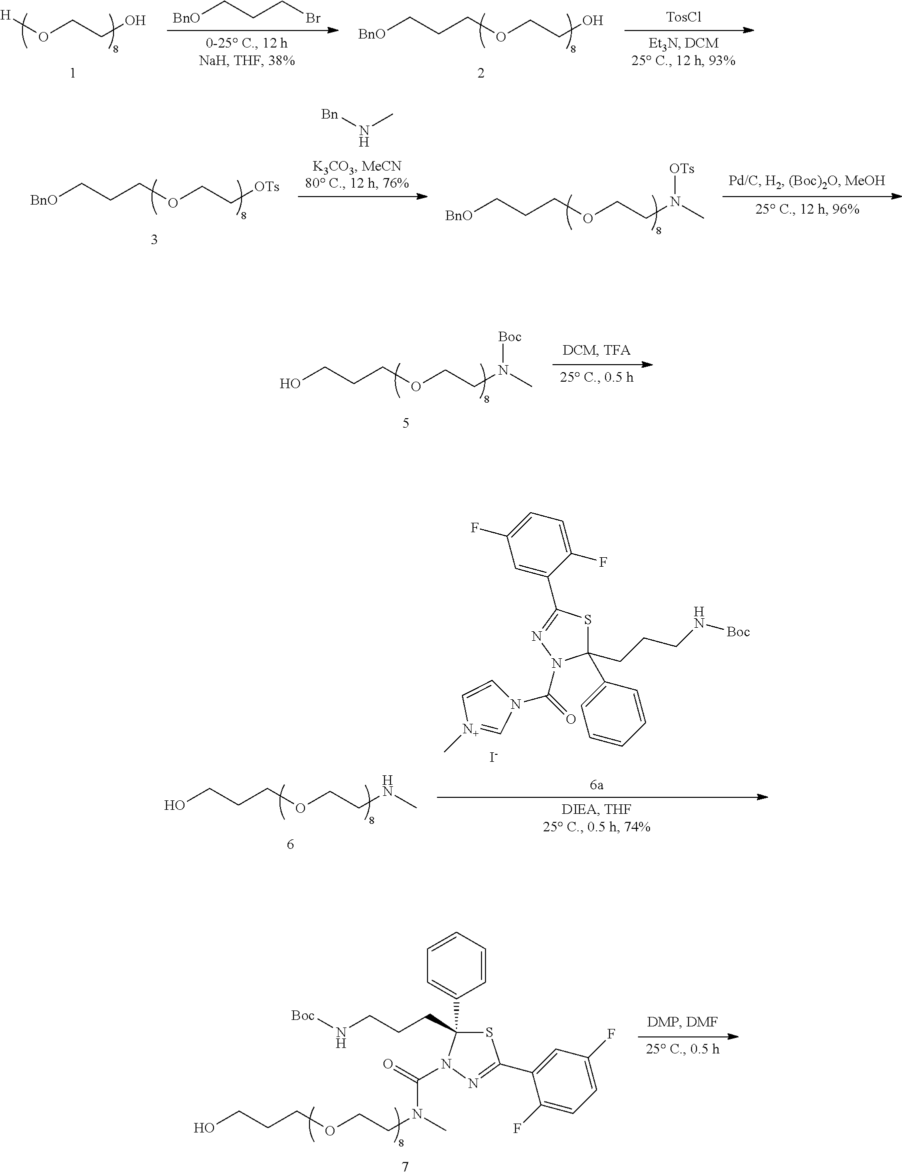

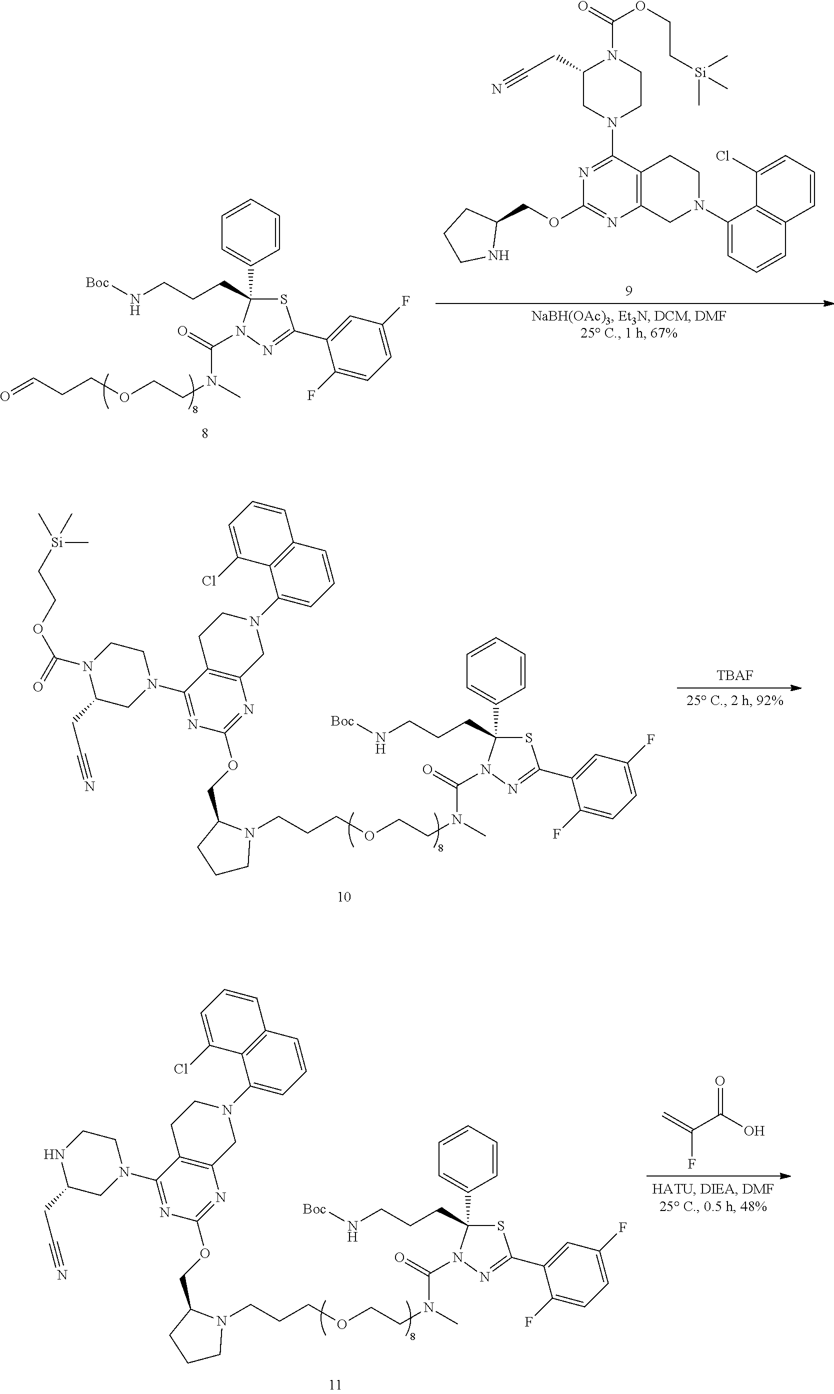

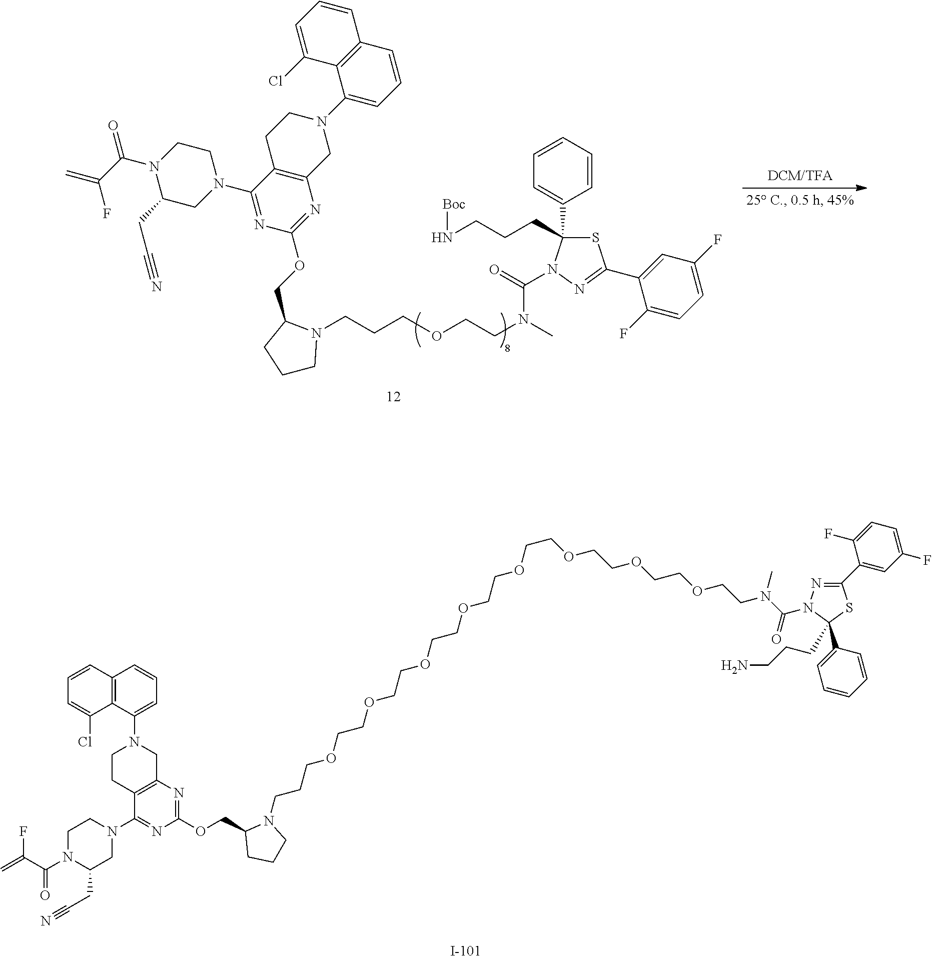

Step 1. To a solution of 2-[2-[2-[2-[2-[2-[2-(2-hydroxyethoxy)ethoxy]ethoxy]ethoxy]ethoxy]ethoxy]ethoxy]ethanol (9.70 g, 26.20 mmol, 1.5 equiv) in THF (30 mL) was added NaH (1.05 g, 26.20 mmol, 60% purity, 1.5 equiv) at 0° C. The mixture was stirred at 25° C. for 1 h. Then 3-bromopropoxymethylbenzene (4 g, 17.4 mmol, 3 mL, 1.0 equiv) was added. The mixture was stirred at 25° C. for 11 h. The mixture was added 20 mL sat.NH₄C₁. The residue was diluted with H₂O (50 mL) and extracted with EA (100 mL×3), dried over Na₂SO₄, filtered and concentrated under reduced pressure to give a residue. The residue was purified by prep-HPLC (column: Kromasil Eternity XT 250*80 mm*10 μm; mobile phase: [water(NH₄HCO₃)-ACN]; B%: 38ACN %-68ACN %, 21 min) to give compound 2-[2-[2-[2-[2-[2-[2-[2-(3-benzyloxypropoxy) ethoxy]ethoxy]ethoxy]ethoxy]ethoxy]ethoxy]ethoxy]ethanol (3.5 g, 38% yield) as a yellow oil.

Step 2. To a solution of 2-[2-[2-[2-[2-[2-[2-[2-(3-benzyloxypropoxy)ethoxy]ethoxy]ethoxy]ethoxy]ethoxy]ethoxy]ethoxy]ethanol (2 g, 3.8 mmol, 1.0 equiv) in DCM (20 mL) was added Et₃N (1.1 g, 11.5 mmol, 1.6 mL, 3.0 equiv) and TosCl (1.4 g, 7.7 mmol, 2.0 equiv). The mixture was stirred at 25° C. for 12 h. The reaction mixture was concentrated to afford crude product. The residue was purified by column chromatography (SiO₂, Petroleum ether/Ethyl acetate=3/1 to 0/1) to give compound 2-[2-[2-[2-[2-[2-[2-[2-(3-benzyloxypropoxy) ethoxy]ethoxy]ethoxy]ethoxy]ethoxy]ethoxy]ethoxy]ethyl 4-methylbenzenesulfonate (2.4 g, 93% yield) as a colorless oil.

Step 3. To a solution of N-methyl-1-phenyl-methanamine (270.1 mg, 2.2 mmol, 287.7 L, 1.5 equiv) and 2-[21[2-[2-[2-[2-[2-(3-benzyloxypropoxy)ethoxy]ethoxy]ethoxy]ethoxy]ethoxy]ethoxy]ethoxy]ethyl 4-methyl benzenesulfonate (1.0 g, 1.49 mmol, 1.0 equiv) in MeCN (5 mL) was added K₂CO₃ (205.42 mg, 1.49 mmol, 1 equiv). The mixture was stirred at 80° C. for 12 h. The residue was purified by prep-HPLC (column: Waters Xbridge C18 150*50 mm*10 μm; mobile phase: [water (NH₄HCO₃)-ACN]; B%: 37%-67%, 11 min) to give compound N-benzyl-2[2-[2-[2-[12-[2-[2-(3-benzyloxypropoxy)ethoxy]ethoxy]ethoxy]ethoxy]ethoxy]ethoxy]ethoxy]-N-methyl-ethanamine (0.7 g, 76% yield) as a yellow oil.

Step 4. To a solution of N-benzyl-2-[2-[2-[2-[2-[2-[2-[2-(3-benzyloxypropoxy)ethoxy]ethoxy]ethoxy]ethoxy]ethoxy]ethoxy]ethoxy]-N-methyl-ethanamine (0.6 g, 964.94 μmol, 1.0 equiv) in MeOH (3 mL) was added (Boc)₂O (421.19 mg, 1.93 mmol, 443.0 μL, 2.0 equiv), Pd/C (0.2 g, 10% purity), and then it was stirred at 25° C. for 12 h under H₂ (15 psi). The reaction mixture was filtered and concentrated under reduced pressure to give compound tert-butyl N-[2-[12[2-[2-[12-[2-[2-(3-hydroxypropoxy)ethoxy]ethoxy]ethoxy]ethoxy]ethoxy]ethoxy]ethoxy]ethyl]-N-methyl-carbamate (0.5 g, 96% yield) as a colorless oil.

Step 5. To a solution of tert-butyl N-[2-[2-[2-[2-[2-[2-[2-[2-(3-hydroxypropoxy) ethoxy]ethoxy]ethoxy]ethoxy]ethoxy]ethoxy]ethoxy]ethyl]-N-methyl-carbamate (140 mg, 258.46 μmol, 1.0 equiv) in DCM (2 mL) was added TFA (1 mL). The mixture was stirred at 25° C. for 0.5 h. The reaction mixture was concentrated under reduced pressure to afford 3-[2-[2-[2-[2-[2-[2-[2-[2-(methylamino)ethoxy]ethoxy]ethoxy]ethoxy]ethoxy]ethoxy]ethoxy]ethoxy]propan-1-ol (143 mg, 99% yield, TFA salt) as a colorless oil.

Step 6. To a solution of tert-butyl N-[3-[5-(2,5-difluorophenyl)-3-(3-BLAH-3-methyl-imidazole-1-carbonyl)-2-phenyl-1,3,4-thiadiazol-2-yl]propyl]carbamate (60 mg, 89.62 μmol, 1.0 equiv) and 3-[2-[2-[2-[2-[2-[2-[2-[2-(methylamino)ethoxy]ethoxy]ethoxy]ethoxy]ethoxy]ethoxy]ethoxy]ethoxy]propan-1-ol (49.79 mg, 89.62 μmol, 1.0 equiv, TFA salt) in THF (2 mL) was added DIEA (34.75 mg, 268.85 μmol, 46.83 μL, 3.0 equiv). The mixture was stirred at 25° C. for 0.5 h. The reaction mixture was concentrated under reduced pressure to afford crude product. The residue was purified by column chromatography (SiO₂, Petroleum ether/Ethyl acetate=5/1 to 0/1) to afford tert-butyl N-[3-[(2S)-5-(2,5-difluorophenyl)-3-[2-[2-[2-[2-[2-[2-[2-[2-(3-hydroxypropoxy)ethoxy]ethoxy]ethoxy]ethoxy]ethoxy]ethoxy]ethoxy]ethyl-methyl-carbamoyl]-2-phenyl-1,3,4-thiadiazol-2-yl]propyl]carbamate (60 mg, 74% yield) as a colorless oil.

Step 7. To a solution of tert-butyl N-[3-[(2S)-5-(2,5-difluorophenyl)-3-[2-[2-[2-[2-[2-[2-[2-1[2-(3-hydroxypropoxy)ethoxy]ethoxy]ethoxy]ethoxy]ethoxy]ethoxy]ethoxy]ethyl-methyl-carbamoyl]-2-phenyl-1,3,4-thiadiazol-2-yl]propyl]carbamate (140 mg, 155.4 μmol, 1.0 equiv) in DMF (1.5 mL) was added Dess-Martin (263.60 mg, 192.41 μL, 4.0 equiv). The mixture was stirred at 25° C. for 3 h. tert-butyl N-[3-[(2S)-5-(2,5-difluorophenyl)-3-[methyl-[2-[2-[2-[2-[2-[2-[2-[2-(3-oxopropoxy)ethoxy]ethoxy]ethoxy]ethoxy]ethoxy]ethoxy]ethoxy]ethyl]carbamoyl]-2-phenyl-1,3,4-thiadiazol-2-yl]propyl]carbamate (135 mg, 96% yield) was obtained as a colorless liquid and used directly for next step.

Step 8. To a solution of tert-butyl N-[3-[(2S)-5-(2,5-difluorophenyl)-3-[methyl-[2-[2-[12[2-[2-[2-[2-(3-oxopropoxy)ethoxy]ethoxy]ethoxy]ethoxy]ethoxy]ethoxy]ethoxy]ethyl]carbamoyl]-2-phenyl-1,3,4-thiadiazol-2-yl]propyl]carbamate (130 mg, 144.60 μmol, 1.0 equiv) in DCM (1 mL) was added Et₃N (146.32 mg, 1.45 mmol, 201.26 μL, 10.0 equiv) and NaBH(OAc)₃ (306.46 mg, 1.45 mmol, 10.0 equiv). Then 2-trimethylsilylethyl(2S)-4-[7-(8-chloro-1-naphthyl)-2-[[(2S)-pyrrolidin-2-yl]methoxy]-6,8-dihydro-5H-pyrido [3, 4-d]pyrimidin-4-yl]-2-(cyanomethyl) piperazine-1-carboxylate (112.3 mg, 144.60 μmol, 1.0 equiv, TFA salt) in DMF (0.5 mL) was added. The mixture was stirred at 25° C. for 1 h. The residue was purified by prep-HPLC (column: Phenomenex Synergi C18 150*25 mm*10 m; mobile phase: [water (FA)-ACN]; B%: 51%-75%, 8 min) to give compound 2-trimethylsilylethyl (2S)-4-[2-[[(2S)-1-[3-[1[2[2-[2-[2-[2-[2-[2-[[(2S)-2-[3-(tert-butoxycarbonylamino)propyl]-5-(2,5-difluorophenyl)-2-phenyl-1,3,4-thiadiazole-3-carbonyl]-methyl-amino]ethoxy]ethoxy]ethoxy]ethoxy]ethoxy]ethoxy]ethoxy]ethoxy]propyl]pyrrolidin-2-yl]methoxy]-7-(8-chloro-1-naphthyl)-6,8-dihydro-5H-pyrido[3,4-d]pyrimidin-4-yl]-2-(cyanomethyl) piperazine-1-carboxylate (150 mg, 67% yield) as a yellow oil.

Step 9. To a solution of 2-trimethylsilylethyl (2S)-4-[2-[[(2S)-1-[3-[2-[2-[2-[2-[2-[2-[2-[2-[[(2S)-2-[3-(tert-butoxycarbonylamino)propyl]-5-(2,5-difluorophenyl)-2-phenyl-1,3,4-thiadiazole-3-carbonyl]-methyl-amino]ethoxy]ethoxy]ethoxy]ethoxy]ethoxy]ethoxy]ethoxy]ethoxy]propyl]pyrrolidin-2-yl]methoxy]-7-(8-chloro-1-naphthyl)-6,8-dihydro-5H-pyrido[3,4-d]pyrimidin-4-yl]-2-(cyanomethyl)piperazine-1-carboxylate (90 mg, 58.24 μmol, 1.0 equiv) in THF (0.5 mL) was added TBAF (0.5 M, 1.29 mL, 11.0 equiv). The mixture was stirred at 25° C. for 2 h. The residue was diluted with H₂O (20 mL) and extracted with EA (20 mL×3). The combined organic layers were dried over anhydrous Na₂SO₄, filtered and concentrated under reduced pressure to afford tert-butyl N-[3-[(2S)-3-[2-[2-[2-[2-[2-[2-[2-[2-[3-[(2S)-2-[[7-(8-chloro-1-naphthyl)-4-[(3S)-3-(cyanomethyl)piperazin-1-yl]-6,8-dihydro-SH-pyrido[3,4-d]pyrimidin-2-yl]oxymethyl]pyrrolidin-1-yl]propoxy]ethoxy]ethoxy]ethoxy]ethoxy]ethoxy]ethoxy]ethoxy]ethyl-methyl-carbamoyl]-5-(2,5-difluorophenyl)-2-phenyl-1,3,4-thiadiazol-2-yl]propyl]carbamate (75 mg, 92% yield) as a yellow oil.

Step 10. To a solution of 2-fluoroprop-2-enoic acid (38.56 mg, 428.24 μmol, 10.0 equiv), tert-butyl N-[3-[(2S)-3-[2-[2-[2-[2-[2-[2-[2-[2-[3-[(2S)-2-[[7-(8-chloro-1-naphthyl)-4-[(3S)-3-(cyanomethyl)piperazin-1-yl]-6,8-dihydro-5H-pyrido[3,4-d]pyrimidin-2-yl]oxymethyl]pyrrolidin-lyl]propoxy]ethoxy]ethoxy]ethoxy]ethoxy]ethoxy]ethoxy]ethoxy]ethyl-methyl-carbamoyl]-5-(2,5-difluorophenyl)-2-phenyl-1,3,4-thiadiazol-2-yl]propyl]carbamate (60 mg, 42.82 μmol, 1.0 equiv) in DMF (0.5 mL) was added DIEA (22.14 mg, 171.29 μmol, 29.84 μL, 4.0 equiv) and HATU (81.41 mg, 214.12 μmol, 5.0 equiv). The mixture was stirred at 25° C. for 0.5 h. The residue was purified by prep-HPLC (column: Phenomenex C18 75*30 mm*3 m; mobile phase: [water (FA)-ACN]; B%: 40%-70%, 7 min) to give compound tert-butyl N-[3-[(2S)-3-[2-[2-[2-[2-[2-[2-[2-[2-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]propoxy]ethoxy]ethoxy]ethoxy]ethoxy]ethoxy]ethoxy]ethoxy]ethyl-methyl-carbamoyl]-5-(2,5-difluorophenyl)-2-phenyl-1,3,4-thiadiazol-2-yl]propyl]carbamate (30 mg, 48% yield) as a yellow solid.

Step 11. To a solution of tert-butyl N-[3-[(2S)-3-[2-[2-[2-[2-[2-[2-[2-[2-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-SH-pyrido1[3,4-d]pyrimidin-2-yl]oxymethyl]pyrrolidin-1-yl]propoxy]ethoxy]ethoxy]ethoxy]ethoxy]ethoxy]ethoxy]ethoxy]ethyl-methyl-carbamoyl]-5-(2,5-difluorophenyl)-2-phenyl-1,3,4-thiadiazol-2-yl]propyl]carbamate (23 mg, 15.6 μmol, 1.0 equiv) in DCM (0.5 mL) was added TFA (0.1 mL), and then it was stirred at 25° C. for 0.5 h. The reaction mixture was concentrated under reduced pressure to remove solvent at 0° C. The residue was purified by prep-HPLC (column: Phenomenex C18 75*30 mm*3 m; mobile phase: [water (FA)-ACN]; B%: 25%-55%, 7 min) to give compound (2S)-2-(3-aminopropyl)-N-[2-[2-[2-2-[2-2-[2-[2-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido [3,4-d]pyrimidin-2-yl]oxymethyl]pyrrolidin-1-yl]propoxy]ethoxy]ethoxy]ethoxy]ethoxy]ethoxy]ethoxy]ethoxy]ethyl]-5-(2,5-difluorophenyl)-N-methyl-2-phenyl-1,3,4-thiadiazole-3-carboxamide (9.99 mg, 45% yield) as a yellow gum. ¹H NMR (400 MHz, CD₃OD): δ8.53 (s, 1H), 7.76-7.89 (m, 2H), 7.69 (m, 1H), 7.46-7.59 (m, 4H), 7.27-7.42 (m, 5H), 7.05-7.18 (m, 2H), 5.22-5.42 (m, 2H), 4.60 (s, 2H), 4.38-4.47 (m, 1H), 4.27-4.37 (m, 3H), 4.18 (d, J=14.13 Hz, 1H), 4.07 (d, J=11.51 Hz, 1H), 3.42-3.86 (m, 39H), 2.96-3.23 (m, 12H), 2.45-2.76 (m, 4H), 2.07-2.19 (m, 2H), 1.76-1.95 (m, 6H). LC-MS: MS (ES′): RT=2.44 min, m/z=687.8 [M/2H⁺]; LCMS method: 25.

### Example 86—Synthesis of Compound I-102

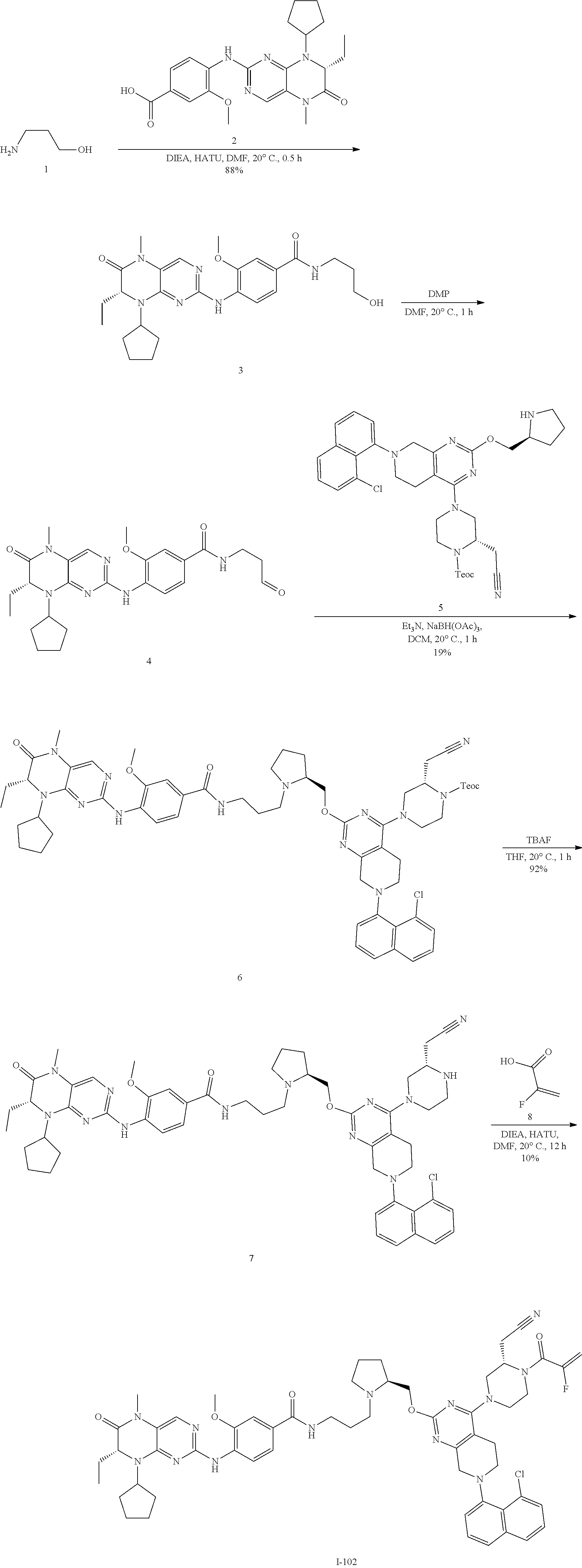

General Information: Compound 2 is a known compound from WO2018/148443, 2018, A1.

Step 1. To a solution of 3-aminopropan-1-ol (70 mg, 940 μmol, 72 μL, 1.0 equiv), 4-[[(7R)-8-cyclopentyl-7-ethyl-5-methyl-6-oxo-7H-pteridin-2-yl]amino]-3-methoxy-benzoic acid (400 mg, 940 μmol, 1.0 equiv) and 4-[[(7R)-8-cyclopentyl-7-ethyl-5-methyl-6-oxo-7H-pteridin-2-yl]amino]-3-methoxy-benzoic acid (400 mg, 940 μmol, 1.0 equiv) in DMF (8 mL) was added HATU (428 mg, 1.13 mmol, 1.2 equiv) and DIEA (364 mg, 2.82 mmol, 491 μL, 3.0 equiv). The mixture was stirred at 20° C. for 0.5 h. The solution was purified by prep-HPLC (column: Waters Xbridge C18 150*50 mm*10%μm; mobile phase: [water(NH₄HCO₃)-ACN]; B%:15%-45%, 11 min) to give 4-[[(7R)-8-cyclopentyl-7-ethyl-5-methyl-6-oxo-7H-pteridin-2-yl]amino]-N-(3-hydroxypropyl)-3-methoxy-benzamide (400 mg, 88% yield).

Step 2. To a solution of 4-[[(7R)-8-cyclopentyl-7-ethyl-5-methyl-6-oxo-7H-pteridin-2-yl]amino]-N-(3-hydroxypropyl)-3-methoxybenzamide (200 mg, 414 μmol, 1.0 equiv) in DMF (1 mL) was added Dess-Martin (439 mg, 1.04 mmol, 320 μL, 2.5 equiv). The mixture was stirred at 20° C. for 1 h. The product was used directly for next step.

Step 3. To a solution of 2-trimethylsilylethyl (2S)-4-[7-(8-chloro-1-naphthyl)-2-[[(2S)-pyrrolidin-2-yl]methoxy]-6,8-dihydro-5Hpyrido [3,4-d]pyrimidin-4-yl]-2-(cyanomethyl) piperazine-1-carboxylate (190 mg, 286 μmol, 1.0 equiv) in DCM (5 mL) was added TEA (290 mg, 2.87 mmol, 399 μL, 10.0 equiv) and NaBH(OAc)₃ (608 mg, 2.87 mmol, 10.0 equiv), then 4-[[(7R)-8-cyclopentyl-7-ethyl-5-methyl-6-oxo-7H-pteridin-2-yl]amino]-3-methoxy-N-(3-oxopropyl)benzamide (137 mg, 286 μmol, 1.0 equiv) was added, the mixture was stirred at 20° C. for 1 h. The solution was purified by prep-HPLC (column: Phenomenex luna C18 150*40 mm* 15 m; mobile phase: [water(FA)-ACN]; B%: 25%-55%, 10 min) to give 2-trimethylsilylethyl (2S)-4-[7-(8-chloro-1-naphthyl)-2-[[(2S)-1-[3-[[4-[[(7R)-8-cyclopentyl-7-ethyl-5-methyl-6-oxo-7Hpteridin-2-yl]amino]-3-methoxy-benzoyl]amino]propyl]pyrrolidin-2-yl]methoxy]-6,8-dihydro-5H-pyrido[3,4-d]pyrimidin-4-yl]-2-(cyanomethyl)piperazine-1-carboxylate (60 mg, 19% yield) as a yellow solid.

Step 4. To a solution of 2-trimethylsilylethyl (2S)-4-[7-(8-chloro-1-naphthyl)-2-[[(2S)-1-[3-[[4-[[(7R)-8-cyclopentyl-7-ethyl-5-methyl-6-oxo-7H-pteridin-2-yl]amino]-3-methoxy-benzoyl]amino]propyl]pyrrolidin-2-yl]methoxy]-6,8-dihydro-5H-pyrido [3,4-d]pyrimidin-4-yl]-2-(cyanomethyl)piperazine-1-carboxylate (50 mg, 44 μmol, 1.0 equiv) in THF (1 mL) was added TBAF (1 M, 133 μL, 3.0 equiv). The mixture was stirred at 20° C. for 12 h. The solution was purified by prep-TLC(DCM/MeOH=10/1) to give N-[3-[(2S)-2-[[7-(8-chloro-1-naphthyl)-4-[(3S)-3-(cyanomethyl)piperazin-1-yl]-6,8-dihydro-5H-pyrido[3,4-d]pyrimidin-2-yl]oxymethyl]pyrrolidin-1-yl]propyl]-4-[[(7R)-8-cyclopentyl-7-ethyl-5-methyl-6-oxo-7H-pteridin-2-yl]amino]-3-methoxy-benzamide (40 mg, 92% yield).

Step 5. To a solution of N-[3-[(2S)-2-[[7-(8-chloro-1-naphthyl)-4-[(3S)-3-(cyanomethyl)piperazin-1-yl]-6,8-dihydro-5H-pyrido[3,4-d]pyrimidin-2-yl]oxymethyl]pyrrolidin-1-yl]propyl]-4-[[(7R)-8-cyclopentyl-7-ethyl-5-methyl-6-oxo-7H-pteridin-2-yl]amino]-3-methoxy-benzamide (40 mg, 40 μmol, 1.0 equiv), 2-fluoroprop-2-enoic acid (4 mg, 41 μmol, 1.0 equiv) in DMF (2 mL) was added HATU (18 mg, 48 μmol, 1.2 equiv) and DIEA (15 mg, 122 μmol, 21 μL, 3.0 equiv). The mixture was stirred at 20° C. for 12 h. The solution was purified by prep-HPLC (column: Phenomenex luna C18 250*50 mm*15 m; mobile phase: [water(FA)-ACN]; B%: 25%-55%, 10 min) to give N-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]propyl]-4-[[(7R)-8-cyclopentyl-7-ethyl-5-methyl-6-oxo-7H-pteridin-2-yl]amino]-3-methoxy-benzamide (5 mg, 10% yield). ¹H NMR (400 MHz, DMSO-d₆): δ8.38 (dd, J=8.0, 4.4 Hz, 1H), 8.27 (s, 1H), 7.90 (d, J=7.6 Hz, 1H), 7.84 (d, J=4.8 Hz, 1H), 7.72 (d, J=8.0 Hz, 1H), 7.61-7.54 (m, 2H), 7.51-7.49 (m, 1H), 7.47-7.41 (m, 2H), 7.40-7.33 (m, 1H), 7.26 (d, J=7.6 Hz, 1H), 5.34 (d, J=7.2 Hz, 2H), 4.22 (d, J=4.0 Hz, 2H), 3.97-3.88 (m, 3H), 3.18 (d, J=7.6 Hz, 4H), 2.78 (s, 2H), 2.19 (s, 1H), 2.01 (d, J=8.0 Hz, 1H), 1.90-1.88 (m, 1H), 1.80-1.73 (m, 4H), 1.61-1.54 (m, 8H), 1.35-1.29 (m, 8H), 1.28-1.24 (m, 4H), 0.94 (t, J=7.2 Hz, 12H). LC-MS: MS (ES′): RT=1.992 min, m/z=1054.4 [M+H⁺]; LCMS method: 25.

### Example 87—Synthesis of Compound I-103

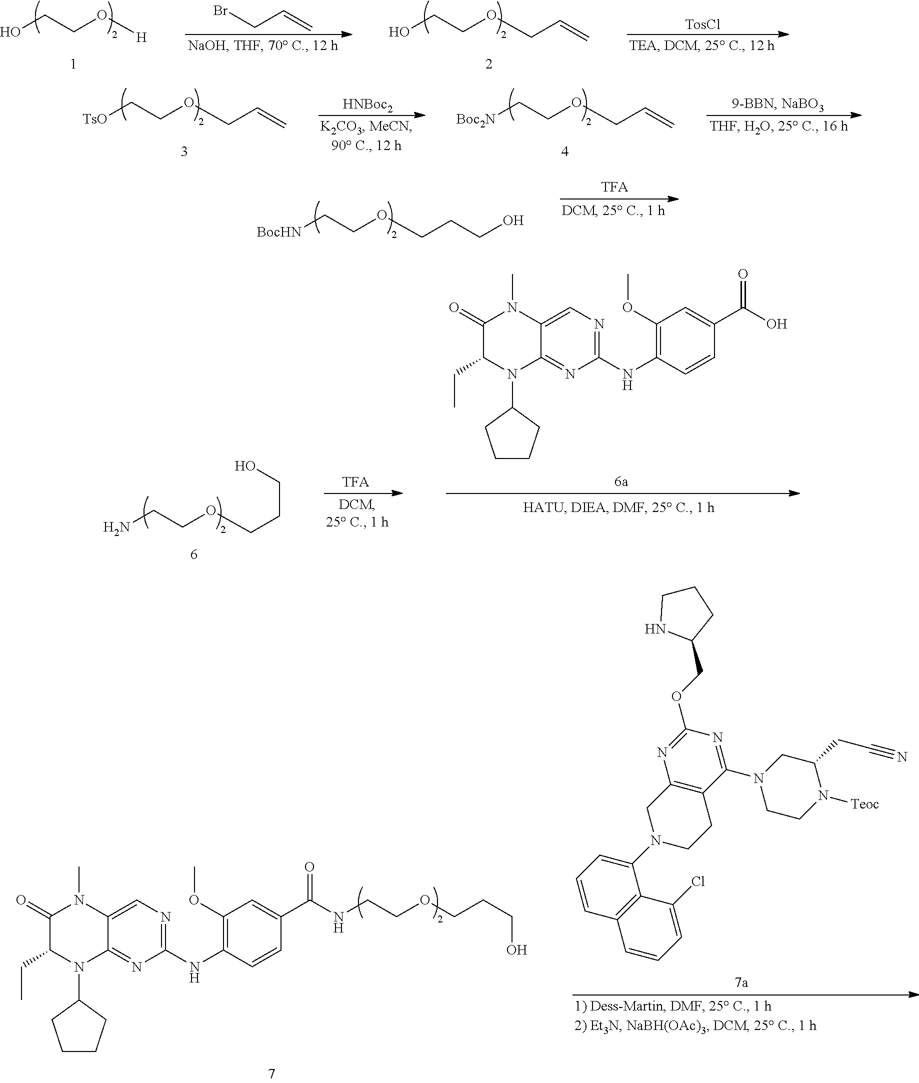

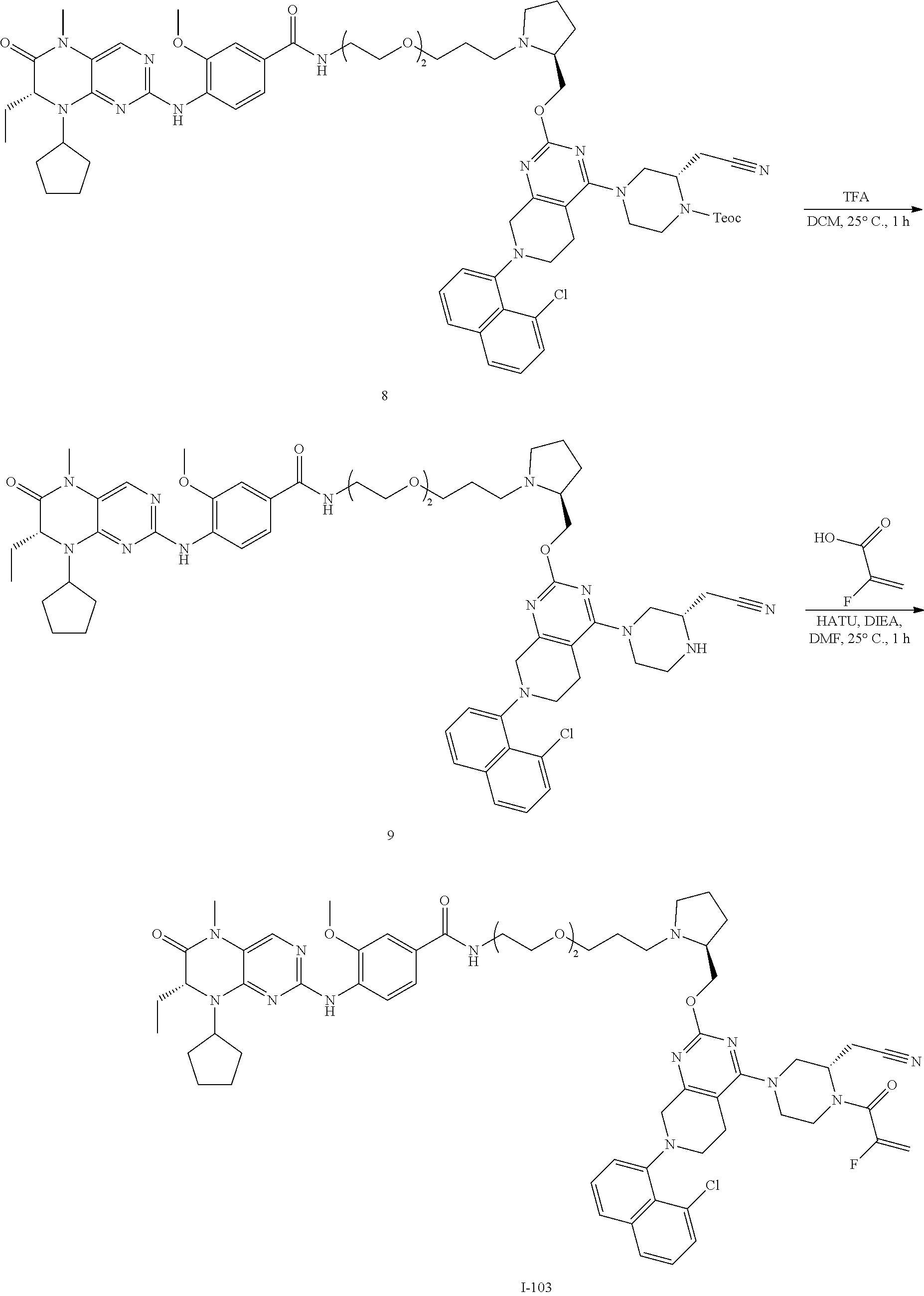

General Information: Compound 6a is a known compound from WO2017/24317.

Step 1. A mixture of 2-(2-hydroxyethoxy)ethanol (15.0 g, 141 mmol, 13.4 mL, 1.0 equiv) and NaOH (5.65 g, 141 mmol, 1.0 equiv) in THF (200 mL). Then 3-bromoprop-1-ene (17.1 g, 141 mmol, 1.0 equiv) was added and the mixture was stirred at 70° C. for 12 h under N₂ atmosphere. The mixture was diluted with water (100 mL) and extracted with EtOAc (3×100 mL). The combined organic layers were washed with brine (100 mL), dried over sodium sulfate, filtered and concentrated. The residue was purified by silica column chromatography on silica gel (Petroleum ether: Ethyl acetate from 20/1 to 3/1) to give 2-(2-allyloxyethoxy)ethanol (11.6 g, 54% yield) as a yellow oil.

Step 2. To a solution of 2-(2-allyloxyethoxy)ethanol (11.6 g, 79.4 mmol, 1.0 equiv) and TEA (24.1 g, 238 mmol, 33.1 mL, 3.0 equiv) in DCM (200 mL) was added 4-methylbenzenesulfonyl chloride (30.3 g, 159 mmol, 2.0 equiv) at 0° C. The mixture was stirred at 25° C. for 12 h. The mixture was diluted with water (100 mL) and extracted with DCM (3×50 mL). The combined organic layers were washed with brine (100 mL), dried over sodium sulfate, filtered and concentrated. The residue was purified by silica column chromatography on silica gel (Petroleum ether: Ethyl acetate from 15/1 to 3/1) to give 2-(2-allyloxyethoxy)ethyl 4-methylbenzenesulfonate (20.0 g, 84% yield) as a colorless oil.

Step 3. To a solution of 2-(2-allyloxyethoxy)ethyl 4-methylbenzenesulfonate (20 g, 66.58 mmol, 1.0 equiv) and tert-butyl N-tert-butoxycarbonylcarbamate (21.7 g, 99.9 mmol, 1.5 equiv) in MeCN (150 mL) was added K₂CO₃ (18.4 g, 133 mmol, 2.0 equiv). The mixture was stirred at 90° C. for 12 h. The mixture was filtered and concentrated under reduced pressure to give a residue. The residue was purified by silica column chromatography (Petroleum ether: Ethyl acetate from 10/1 to 3/1) to give tert-butyl N-[2-(2-allyloxyethoxy)ethyl]-N-tert-butoxycarbonyl-carbamate (13.4 g, 58% yield) as a colorless oil.

Step 4. 9—BBN (0.5 M, 86.8 mL, 3.0 equiv) in THF (30 mL) was added to tert-butyl N-[2-(2-allyloxyethoxy)ethyl]-N-tert-butoxycarbonyl-carbamate (5.0 g, 14.5 mmol, 1.0 equiv) under the atmosphere of N₂ at 0° C. and the mixture was stirred at 25° C. for 4 h. Then sodium; 3-oxidodioxaborirane tetrahydrate (8.9 g, 57.9 mmol, 11.2 mL, 4.0 equiv) in H₂O (20 mL) was added and the mixture was stirred at 25° C. for 12 h. The mixture was diluted with water (50 mL) and extracted with EtOAc (3×50 mL). The combined organic layers were washed with brine (50 mL), dried over sodium sulfate, filtered and concentrated. The residue was purified by prep-HPLC (column: Kromasil Eternity XT 250*80 mm*10 m; mobile phase: [water(NH₄HCO₃)-ACN]; B%: 35ACN %-65ACN %, 18 min) to give tert-butyl N-tert-butoxycarbonyl-N-[2-[2-(3-hydroxypropoxy) ethoxy]ethyl]carbamate (5.0 g, 95% yield) as a yellow oil.

Step 5. To a solution of tert-butyl N-tert-butoxycarbonyl-N-[2-[2-(3-hydroxypropoxy) ethoxy]ethyl]carbamate (650 mg, 1.79 mmol, 1.0 equiv) in DCM (4 mL) was added TFA (1.0 mL). The mixture was stirred at 25° C. for 1 h. The reaction mixture was concentrated under reduced pressure to give 3-[2-(2-aminoethoxy)ethoxy]propan-1-ol (290 mg, crude) as a colorless oil without purification.

Step 6. To a solution of 3-[2-(2-aminoethoxy)ethoxy]propan-1-ol (172 mg, 1.06 mmol, 1.5 equiv) and 4-[[(7R)-8-cyclopentyl-7-ethyl-5-methyl-6-oxo-7H-pteridin-2-yl]amino]-3-methoxy-benzoic acid (300 mg, 705 μmol, 1.0 equiv) in DMF (5 mL) was added DIEA (365 mg, 2.82 mmol, 4.0 equiv) and HATU (322 mg, 846 μmol, 1.2 equiv). The mixture was stirred at 25° C. for 1 h. The residue was purified by prep-HPLC (column: Phenomenex luna C18 250*50 mm*15 m; mobile phase: [water(FA)-ACN]; B%: 17%-47%, 10 min) to give 4-[[(7R)-8-cyclopentyl-7-ethyl-5-methyl-6-oxo-7H-pteridin-2-yl]amino]-N—[2-[2-(3-hydroxypropoxy) ethoxy]ethyl]-3-methoxy-benzamide (340 mg, 85% yield) was obtained as a colorless oil.

Step 7. To a solution of 4-[[(7R)-8-cyclopentyl-7-ethyl-5-methyl-6-oxo-7H-pteridin-2-yl]amino]-N-[2-[2-(3-hydroxypropoxy)ethoxy]ethyl]-3-methoxy-benzamide (147 mg, 258 μmol, 2.0 equiv) in DMF (3 mL) was added Dess-Martin (219 mg, 515 μmol, 4.0 equiv) and stirred at 25° C. for 1 h. Then the solution was added to a solution of 2-trimethylsilylethyl (2S)-4-[7-(8-chloro-1-naphthyl)-2-[ [(2S)-pyrrolidin-2-yl]methoxy]-6,8-dihydro-SH-pyrido[3,4-d]pyrimidin-4-yl]-2-(cyanomethyl)piperazine-1-carboxylate (100 mg, 129 μmol, 1.0 equiv), TEA (130 mg, 1.29 mmol, 10.0 equiv) and NaBH(OAc)₃ (410 mg, 1.93 mmol, 15.0 equiv) in DCM (10 mL) at 0° C. The mixture was stirred at 25° C. for 1 h. The mixture was diluted with water (10 mL) and extracted with DCM (3×10 mL). The combined organic layers were washed with brine (10 mL), dried over sodium sulfate, filtered and concentrated. The residue was purified by prep-HPLC (column: Unisil 3-100 C18 Ultra 150*50 mm*3 m; mobile phase: [water(FA)-ACN]; B%: 30%-60%, 10 min) to give 2-trimethylsilylethyl (2S)-4-[7-(8-chloro-1-naphthyl)-2-[[(2S)-1-[3-[[22-[[4-[[(7R)-8-cyclopentyl-7-ethyl-5-methyl-6-oxo-7H-pteridin-2-yl]amino]-3-methoxybenzoyl]amino]ethoxy]ethoxy]propyl]pyrrolidin-2-yl]methoxy]-6,8-dihydro-5H-pyrido[3,4-d]pyrimidin-4-yl]-2-(cyanomethyl)piperazine-1-carboxylate (55 mg, 35% yield) as a brown solid.

Step 8. To a solution of 2-trimethylsilylethyl (2S)-4-[7-(8-chloro-1-naphthyl)-2-[[(2S)-1-[3-[[22-[[4-[[(7R)-8-cyclopentyl-7-ethyl-5-methyl-6-oxo-7H-pteridin-2-yl]amino]-3-methoxy-benzoyl]amino]ethoxy]ethoxy]propyl]pyrrolidin-2-yl]methoxy]-6,8-dihydro-5H-pyrido[3,4-d]pyrimidin-4-yl]-2-(cyanomethyl)piperazine-1-carboxylate (40 mg, 33 μmol, 1.0 equiv) in DCM (1 mL) was added TFA (0.2 mL). The mixture was stirred at 25° C. for 1 h and concentrated under reduced pressure to give N-[2-[2-[3-[(2S)-2-[[7-(8-chloro-1-naphthyl)-4-[(3S)-3-(cyanomethyl)piperazin-1-yl]-6,8-dihydro-5H-pyrido[3,4-d]pyrimidin-2-yl]oxymethyl]pyrrolidin-1-yl]propoxy]ethoxy]ethyl]-4-[[(7R)-8-cyclopentyl-7-ethyl-5-methyl-6-oxo-7H-pteridin-2-yl]amino]-3-methoxy-benzamide (35 mg, TFA salt) was obtained as a colorless oil without purification for the next step.

Step 9. To a solution of N-[2-[2-[3-[(2S)-2-[[7-(8-chloro-1-naphthyl)-4-[(3S)-3-(cyanomethyl)piperazin-1-yl]-6,8-dihydro-5H-pyrido[3,4-d]pyrimidin-2-yl]oxymethyl]pyrrolidin-1-yl]propoxy]ethoxy]ethyl]-4-[[(7R)-8-cyclopentyl-7-ethyl-5-methyl-6-oxo-7H-pteridin-2-yl]amino]-3-methoxy-benzamide (35 mg, 33 μmol, 1.0 equiv, TFA salt) and 2-fluoroprop-2-enoic acid (5.9 mg, 66 μmol, 2.0 equiv) in DMF (1 mL)was added DIEA (17 mg, 132 μmol, 4.0 equiv) and HATU (14 mg, 36 μmol, 1.1 equiv). The mixture was stirred at 25° C. for 1 h. The residue was purified by prep-HPLC (column: Unisil 3-100 C18 Ultra 150*50 mm*3 m; mobile phase: [water(FA)-ACN]; B%: 17%-47%, 10 min) to give N-[2-[2-[3-[(2S)-2-1[[7-(8-chloro-1-naphthyl)-4-[(3S)-3-(cyanomethyl)-4-(2-fluoroprop-2-enoyl)piperazin-1-yl]-6,8-dihydro-5H-pyrido1[3,4-d]pyrimidin-2-yl]oxymethyl]pyrrolidin-1-yl]propoxy]ethoxy]ethyl]-4-[1[(7R)-8-cyclopentyl-7-ethyl-5-methyl-6-oxo-7H-pteridin-2-yl]amino]-3-methoxy-benzamide (14 mg, 35% yield) was obtained as a yellow solid. ¹H NMR (400 MHz, CD₃OD): δ8.51-8.41 (m, 2H), 7.84-7.76 (m, 2H), 7.66 (d, J=8.2 Hz, 1H), 7.53-7.41 (m, 4H), 7.39-7.21 (m, 2H), 5.40-5.24 (m, 4H), 4.59 (s, 15H), 4.52-4.40 (m, 1H), 4.33-4.24 (m, 1H), 3.96 (d, J=1.2 Hz, 2H), 3.68-3.52 (m, 12H), 3.25-3.02 (m, 8H), 2.74-2.61 (m, 1H), 2.05 (s, 3H), 2.01-1.63 (m, 12H), 0.83 (t, J=3.6 Hz, 3H). LC-MS: MS (ES′): RT=2.057 min, m/z=1142.4 [M+H⁺]; LCMS method: 25.

### Example 88—Synthesis of Compound I-104

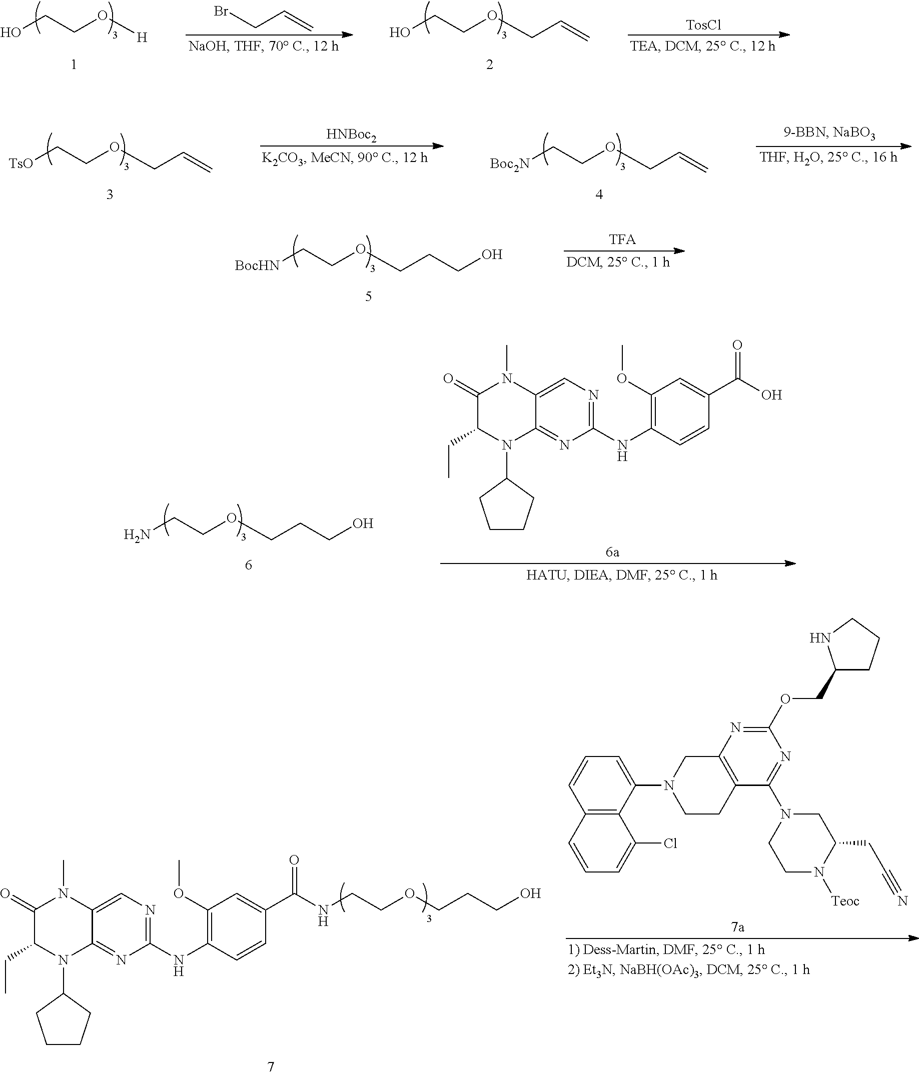

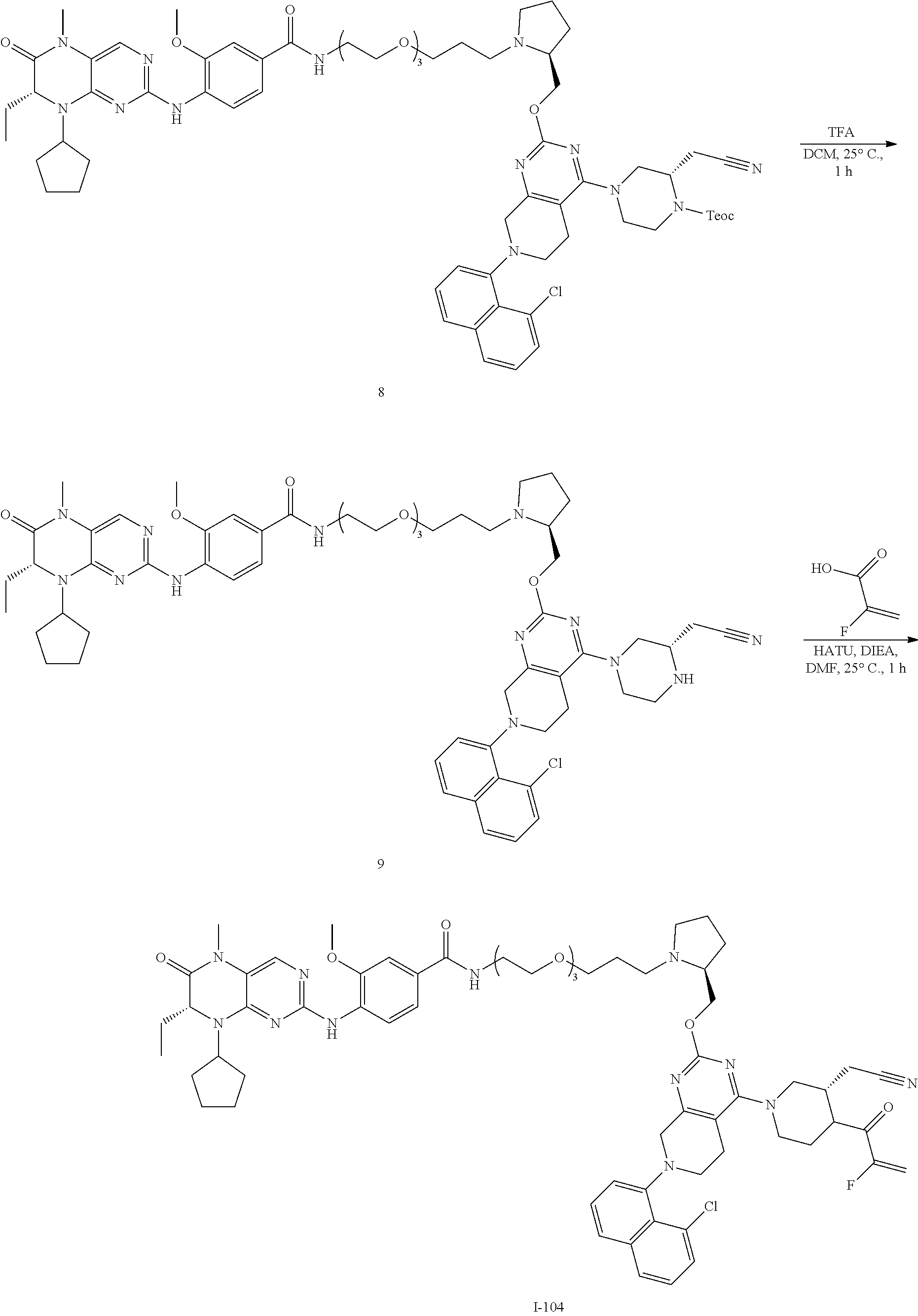

General Information: Compound 6a is a known compound from WO2017/24317, 2017, A2.

Step 1. A mixture of 2-[2-(2-hydroxyethoxy)ethoxy]ethanol (15.0 g, 100 mmol, 13.4 mL, 1.0 equiv) and NaOH (4.00 g, 100 mmol, 1.0 equiv) in THF (200 mL) was added 3-bromoprop-1-ene (12.1 g, 100 mmol, 1.0 equiv), and then it was stirred at 70° C. for 12 h under N₂ atmosphere. The mixture was diluted with water (100 mL) and extracted with EtOAc (3×100 mL). The combined organic layers were washed with brine (100 mL), dried over sodium sulfate, filtered and concentrated. The residue was purified by silica column chromatography on silica gel (Petroleum ether: Ethyl acetate from 20/1 to 3/1) to give 2-[2-(2-allyloxyethoxy)ethoxy]ethanol (11.5 g, 60% yield) as a yellow oil.

Step 2. To a solution of 2-[2-(2-allyloxyethoxy)ethoxy]ethanol (11.5 g, 60.5 mmol, 1.0 equiv) and TEA (18.4 g, 181 mmol, 25.2 mL, 3.0 equiv) in DCM (200 mL) was added 4-methylbenzenesulfonyl chloride (23.1 g, 121 mmol, 2.0 equiv) at 0° C. The mixture was stirred at 25° C. for 12 h. The mixture was diluted with water (100 mL) and extracted with DCM (3×50 mL). The combined organic layers were washed with brine (100 mL), dried over sodium sulfate, filtered and concentrated. The residue was purified by silica column chromatography (Petroleum ether: Ethyl acetate from 15/1 to 3/1) to give 2-[2-(2-allyloxyethoxy)ethoxy]ethyl 4-methylbenzenesulfonate (19.0 g, 91% yield) as a colorless oil.

Step 3. To a solution of 2-[2-(2-allyloxyethoxy)ethoxy]ethyl 4-methylbenzenesulfonate (19.0 μg, 55.2 μmmol, 1.0 equiv) and tert-butyl N-tert-butoxycarbonylcarbamate (18.0 g, 82.7 mmol, 1.5 equiv) in MeCN (150 mL) was added K₂CO₃ (22.9 g, 166 mmol, 3.0 equiv). The mixture was stirred at 90° C. for 12 h. The mixture was filtered and concentrated under reduced pressure to give a residue. The residue was purified by silica column chromatography on silica gel (Petroleum ether: Ethyl acetate from 20/1 to 5/1) to give tert-butyl N—[2-[2-(2-allyloxyethoxy)ethoxy]ethyl]-N-tert-butoxycarbonyl-carbamate (13.4 g, 62% yield) as a colorless oil.

Step 4. 9—BBN (0.5 M, 77.1 mL, 3.0 equiv) in THF (30 mL) was added to tert-butyl N-[2-[2-(2-allyloxyethoxy)ethoxy]ethyl]-N-tert-butoxycarbonyl-carbamate (5.0 g, 12.8 mmol, 1.0 equiv) under the atmosphere of N₂ at 0° C. and the mixture was stirred at 25° C. for 4 h. Then sodium; 3-oxidodioxaborirane; tetrahydrate (7.9 g, 51.4 mmol, 9.88 mL, 4.0 equiv) in H₂O (20 mL) was added and the mixture was stirred at 25° C. for 12 h. The mixture was diluted with water (50 mL) and extracted with EtOAc (3×50 mL). The combined organic layers were washed with brine (50 mL), dried over sodium sulfate, filtered and concentrated. The residue was purified by prep-HPLC (column: Kromasil Eternity XT 250*80 mm*10 m; mobile phase: [water(NH4HCO₃)-ACN]; B%: 30ACN %-60ACN %, 18 min) to give tert-butyl N-tert-butoxycarbonyl-N-[2-[2-[2-(3-hydroxypropoxy)ethoxy]ethoxy]ethyl]carbamate (4.0 g, 76% yield) as a colorless oil.

Step 5. To a solution of tert-butyl N-tert-butoxycarbonyl-N-[2-[2-[2-(3-hydroxypropoxy)ethoxy]ethoxy]ethyl]carbamate (500 mg, 1.23 mmol, 1.0 equiv) in DCM (4 mL) was added TFA (1.0 mL). The mixture was stirred at 25° C. for 1 h. The reaction mixture was concentrated under reduced pressure to give 3-[2-[2-(2-aminoethoxy)ethoxy]ethoxy]propan-1-ol (250 mg, TFA salt) as a colorless oil without further purification.

Step 6. To a solution of 3-[2-[2-(2-aminoethoxy)ethoxy]ethoxy]propan-1-ol (220 mg, 1.06 mmol, 1.5 equiv) and 4-[[(7R)-8-cyclopentyl-7-ethyl-5-methyl-6-oxo-7H-pteridin-2-yl]amino]-3-methoxy-benzoic acid (300 mg, 705 μmol, 1.0 equiv) inDMF (5 mL) was added DIEA (365 mg, 2.82 mmol, 4.0 equiv) and HATU (322 mg, 846 μmol, 1.2 equiv), and then it was stirred at 25° C. for 1 h. The reaction mixture was concentrated under reduced pressure to give a residue. The residue was purified by prep-HPLC (column: Phenomenex luna C18 150*40 mm* 15 m; mobile phase: [water(FA)-ACN]; B%: 13%-43%, 10 min) to give 4-[[(7R)-8-cyclopentyl-7-ethyl-5-methyl-6-oxo-7H-pteridin-2-yl]amino]-N-[2-[2-1[2-(3-hydroxypropoxy)ethoxy]ethoxy]ethyl]-3-methoxy-benzamide (300 mg, 69% yield) as a colorless oil.

Step 7. To a solution of 4-[[(7R)-8-cyclopentyl-7-ethyl-5-methyl-6-oxo-7H-pteridin-2-yl]amino]-N-[2-[2-(3-hydroxypropoxy)ethoxy]ethyl]-3-methoxy-benzamide (158 mg, 258 μmol, 2.0 equiv) in DMF (3 mL) was added Dess-Martin (219 mg, 515 μmol, 4.0 equiv) and stirred at 25° C. for 1 h. Then the solution was added to a solution of 2-trimethylsilylethyl (2S)-4-[7-(8-chloro-1-naphthyl)-2-[ [(2S)-pyrrolidin-2-yl]methoxy]-6,8-dihydro-5H-pyrido[3,4-d]pyrimidin-4-yl]-2-(cyanomethyl)piperazine-1-carboxylate (100 mg, 129 μmol, 1.0 equiv), TEA (130 mg, 1.29 mmol, 10.0 equiv) and NaBH(OAc)₃ (410 mg, 1.93 mmol, 15.0 equiv) in DCM (10 mL) at 0° C. The mixture was stirred at 25° C. for 1 h. The mixture was diluted with water (10 mL) and extracted with DCM (3×10 mL). The combined organic layers were washed with brine (10 mL), dried over sodium sulfate, filtered and concentrated. The residue was purified by prep-HPLC (column: Unisil 3-100 C18 Ultra 150*50 mm*3 m; mobile phase: [water(FA)-ACN]; B%: 30%-60%, 10 min) to give 2-trimethylsilylethyl (2S)-4-[7-(8-chloro-1-naphthyl)-2-[[(2S)-1-[3-[1[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]propyl]pyrrolidin-2-yl]methoxy]-6,8-dihydro-5H-pyrido[3,4-d]pyrimidin-4-yl]-2-(cyanomethyl)piperazine-1-carboxylate (35 mg, 22% yield) as a brown solid.

Step 8. To a solution of 2-trimethylsilylethyl (2S)-4-[7-(8-chloro-1-naphthyl)-2-[[(2S)-1-[3-[1[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]propyl]pyrrolidin-2-yl]methoxy]-6,8-dihydro-5H-pyrido[3,4-d]pyrimidin-4-yl]-2-(cyanomethyl)piperazine-1-carboxylate (35 mg, 28 μmol, 1.0 equiv) in DCM (1 mL) was added TFA (0.2 mL). The mixture was stirred at 25° C. for 1 h and concentrated under reduced pressure to give N-[2-[2-[2-[3-[(2S)-2-[[7-(8-chloro-1-naphthyl)-4-[(3S)-3-(cyanomethyl)piperazin-1-yl]-6,8-dihydro-5H-pyrido[3,4-d]pyrimidin-2-yl]oxymethyl]pyrrolidin-1-yl]propoxy]ethoxy]ethoxy]ethyl]-4-[[(7R)-8-cyclopentyl-7-ethyl-5-methyl-6-oxo-7H-pteridin-2-yl]amino]-3-methoxy-benzamide (30 mg, TFA salt) as a colorless oil without purification for the next step.

Step 9. To a solution of N-[2-[2-[2-[3-[(2S)-2-[[7-(8-chloro-1-naphthyl)-4-[(3S)-3-(cyanomethyl)piperazin-1-yl]-6,8-dihydro-SH-pyrido[3,4-d]pyrimidin-2-yl]oxymethyl]pyrrolidin-1-yl]propoxy]ethoxy]ethoxy]ethyl]-4-[[(7R)-8-cyclopentyl-7-ethyl-5-methyl-6-oxo-7H-pteridin-2-yl]amino]-3-methoxy-benzamide (30 mg, 37 μmol, 1.0 equiv) and 2-fluoroprop-2-enoic acid (4.8 mg, 54 μmol, 2.0 equiv) in DMF (1 mL) was added DIEA (14 mg, 108 μmol, 4.0 equiv) and HATU (11 mg, 30 μmol, 1.1 equiv). The mixture was stirred at 25° C. for 1 h. The residue was purified by prep-HPLC (column: Unisil 3-100 C18 Ultra 150*50 mm*3 m; mobile phase: [water(FA)-ACN]; B%: 15%-45%, 10 min) to give N-[2-[2-[2-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]propoxy]ethoxy]ethoxy]ethyl]-4-[[(7R)-8-cyclopentyl-7-ethyl-5-methyl-6-oxo-7H-pteridin-2-yl]amino]-3-methoxy-benzamide (7.5 mg, 20% yield) was obtained as a yellow solid. ¹H NMR (400 MHz, CDCl₃): δ8.54 (d, J=8.4 Hz, 1H), 7.78-7.73 (m, 1H), 7.68 (s, 1H), 7.66-7.57 (m, 2H), 7.52 (d, J=7.5 Hz, 1H), 7.48-7.40 (m, 2H), 7.40-7.31 (m, 2H), 7.26-7.19 (m, 1H), 6.96-6.88 (m, 1H), 5.48-5.32 (m, 1H), 5.28-5.20 (m, 1H), 4.55-4.33 (m, 4H), 4.30-4.02 (m, 4H), 3.97 (s, 3H), 3.69-3.51 (m, 18H), 3.33 (s, 3H), 3.28-3.19 (m, 2H), 3.11-3.06 (m, 1H), 3.01-2.76 (m, 4H), 2.65-2.57 (m, 1H), 2.20-2.12 (m, 2H), 1.98 (d, J=8.6 Hz, 4H), 1.88-1.79 (m, 12H), 0.88 (t, J=7.6 Hz, 3H) LC-MS: MS (ES′): RT=2.045 min, m/z=1186.4 [M+H⁺]; LCMS method: 25

### Example 89—Synthesis of Compound I-105

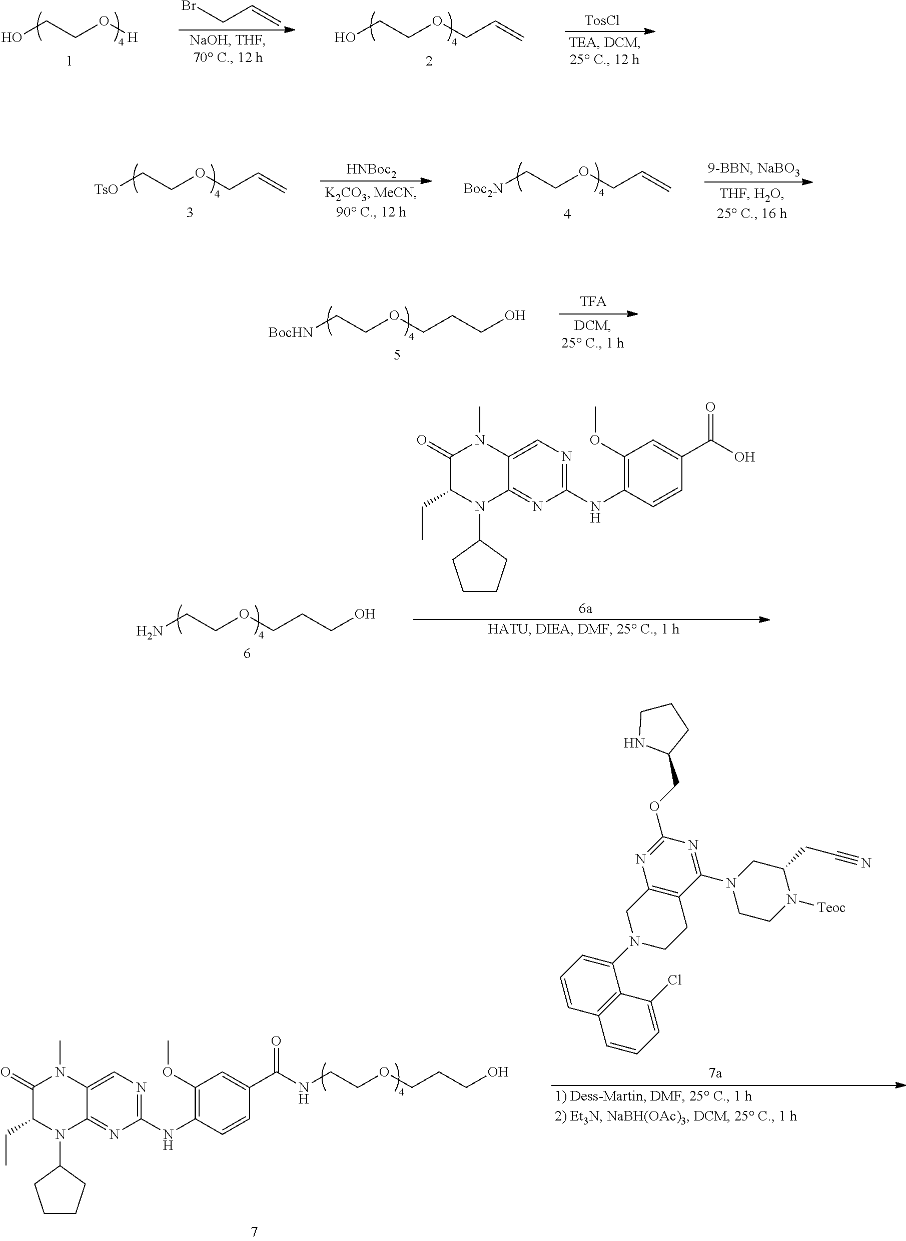

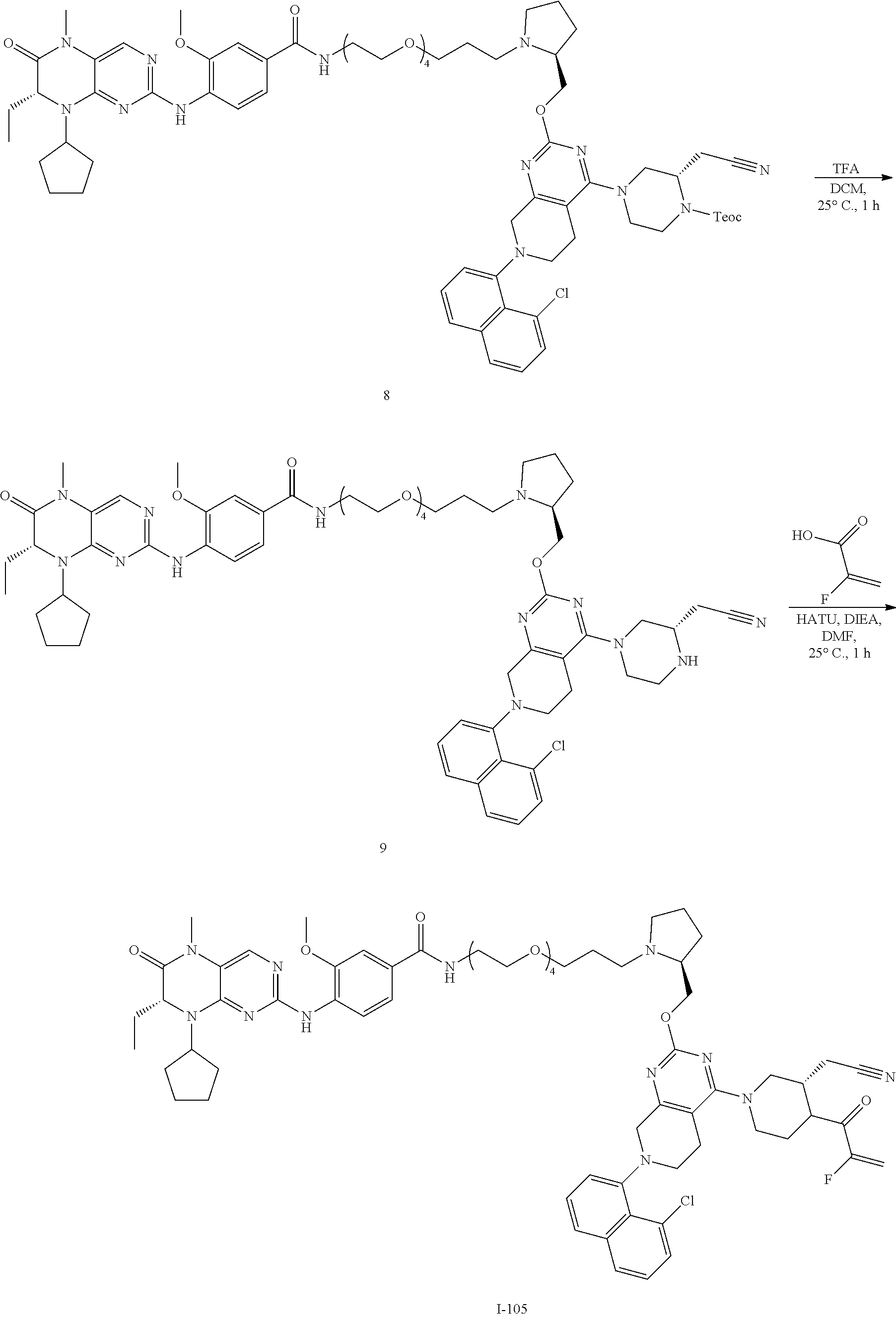

General Information: Compound 6a is a known compound from WO2017/24317, 2017, A2.

Step 1. To a mixture of 2-[12-[2-(2-hydroxyethoxy)ethoxy]ethoxy]ethanol (15.0 g, 77.2 mmol, 13.3 mL, 1.0 equiv) and NaOH (3.10 g, 77.2 mmol, 1.0 equiv) in THF (200 mL) was added 3-bromoprop-1-ene (9.34 g, 77.2 mmol, 1.0 equiv), and then it was stirred at 70° C. for 12 h under N₂ atmosphere. The mixture was diluted with water (100 mL) and extracted with EtOAc (3×100 mL). The combined organic layers were washed with brine (100 mL), dried over sodium sulfate, filtered and concentrated. The residue was purified by silica column chromatography on silica gel (Petroleum ether: Ethyl acetate from 20/1 to 3/1) to give 2-[12-[12-(2-allyloxyethoxy)ethoxy]ethoxy]ethanol (10.2 g, 56% yield) as a yellow oil.

Step 2. To a solution of 2-[2-[2-(2-allyloxyethoxy)ethoxy]ethoxy]ethanol (10.2 g, 43.5 mmol, 1.0 equiv) and TEA (13.2 g, 131 mmol, 18.2 mL, 3.0 equiv) in DCM (200 mL)was added 4-methylbenzenesulfonyl chloride (16.6 g, 87.1 mmol, 2.0 equiv) at 0° C. The mixture was stirred at 25° C. for 12 h. The mixture was diluted with water (100 mL) and extracted with DCM (3×50 mL). The combined organic layers were washed with brine (100 mL), dried over sodium sulfate, filtered and concentrated. The residue was purified by silica column chromatography on silica gel (Petroleum ether: Ethyl acetate from 10/1 to 3/1) to give 2-[2-(2-allyloxyethoxy)ethoxy]ethyl 4-methylbenzenesulfonate (14.0 g, 83% yield) as a colorless oil.

Step 3. To a solution of 2-[2-[2-(2-allyloxyethoxy)ethoxy]ethoxy]ethyl 4-methylbenzenesulfonate (14.0 μg, 36.1 μmmol, 1 equiv) and tert-butyl N-tert-butoxycarbonylcarbamate (11.7 g, 54.1 mmol, 1.5 equiv) in MeCN (150 mL) was added K₂CO₃ (14.9 g, 108 mmol, 3.0 equiv). The mixture was stirred at 90° C. for 12 h. The mixture was filtered and concentrated under reduced pressure to give a residue. The residue was purified by silica column chromatography on silica gel (Petroleum ether: Ethyl acetate from 30/1 to 5/1) to give tert-butyl N-[2-[2-[2-(2-allyloxyethoxy)ethoxy]ethoxy]ethyl]-N-tert-butoxycarbonyl-carbamate (14.5 g, 93% yield) as a colorless oil.

Step 4. 9—BBN (0.5 M, 69.2 mL, 3.0 equiv) in THF (30 mL) was added to tert-butyl N—[2-[2-[2-(2-allyloxyethoxy)ethoxy]ethoxy]ethyl]-N-tert-butoxycarbonyl-carbamate (5.0 g, 11.5 mmol, 1.0 equiv) under the atmosphere of N₂ at 0° C. and the mixture was stirred at 25° C. for 4 h, and then sodium; 3-oxidodioxaborirane; tetrahydrate (7.1 g, 46.1 mmol, 8.87 mL, 4.0 equiv) in H₂O (20 mL) was added and the mixture was stirred at 25° C. for 12 h. The mixture was diluted with water (50 mL) and extracted with EtOAc (3×50 mL). The combined organic layers were washed with brine (50 mL), dried over sodium sulfate, filtered and concentrated. The residue was purified by prep-HPLC (column: Kromasil Eternity XT 250*80 mm*10 μm; mobile phase: [water(NH4HCO₃)-ACN]; B%: 36ACN %-66ACN %, 15 min) to give tert-butyl N-tert-butoxycarbonyl-N-[2-[2-[2-[2-(3-hydroxypropoxy)ethoxy]ethoxy]ethoxy]ethyl]carbamate (5.0 g, 96% yield) as a colorless oil.

Step 5. To a solution of tert-butyl N-tert-butoxycarbonyl-N-[2-[2-[2-[2-(3-hydroxypropoxy)ethoxy]ethoxy]ethoxy]ethyl]carbamate (500 mg, 1.11 mmol, 1.0 equiv) in DCM (4 mL)was added TFA (1.0 mL). The mixture was stirred at 25° C. for 1 h. The reaction mixture was concentrated under reduced pressure to give 3-[2-[2-[2-(2-aminoethoxy)ethoxy]ethoxy]ethoxy]propan-1-ol (270 mg, TFA salt) as a colorless oil without further purification.

Step 6. To a solution of 3-[2-[2-[2-(2-aminoethoxy)ethoxy]ethoxy]ethoxy]propan-1-ol (266 mg, 1.06 mmol, 1.5 equiv, TFA salt) and 4-[[(7R)-8-cyclopentyl-7-ethyl-5-methyl-6-oxo-7H-pteridin-2-yl]amino]-3-methoxy-benzoic acid (300 mg, 705 μmol, 1.0 equiv) in DMF (5 mL) was added DIEA (365 mg, 2.82 mmol, 4.0 equiv) and HATU (322 mg, 846 μmol, 1.2 equiv). The mixture was stirred at 25° C. for 1 h. The reaction mixture was concentrated under reduced pressure to give a residue. The residue was purified by prep-HPLC (column: Phenomenex luna C18 150*40 mm*15 m; mobile phase: [water(FA)-ACN]; B%: 13%-43%, 10 min) to give 4-[[(7R)-8-cyclopentyl-7-ethyl-5-methyl-6-oxo-7H-pteridin-2-yl]amino]-N-[2-[2-[2-[2-(3-hydroxypropoxy)ethoxy]ethoxy]ethoxy]ethyl]-3-methoxy-benzamide (300 mg, 65% yield) as a colorless oil.

Step 7. To a solution of 4-[[(7R)-8-cyclopentyl-7-ethyl-5-methyl-6-oxo-7H-pteridin-2-yl]amino]-N-[2-[2-[2-2-(3-hydroxypropoxy)ethoxy]ethoxy]ethoxy]ethyl]-3-methoxy-benzamide (170 mg, 258 μmol, 2.0 equiv) in DMF (3 mL) was added Dess-Martin (219 mg, 515 μmol, 4.0 equiv) and stirred at 25° C. for 1 h. Then the solution was added to a solution of 2-trimethylsilylethyl (2S)-4-[7-(8-chloro-1-naphthyl)-2-[[(2S)-pyrrolidin-2-yl]methoxy]-6,8-dihydro-5H-pyrido[3,4-d]pyrimidin-4-yl]-2-(cyanomethyl)piperazine-1-carboxylate (100 mg, 129 μmol, 1.0 equiv), TEA (130 mg, 1.29 mmol, 10.0 equiv) and NaBH(OAc)₃ (410 mg, 1.93 mmol, 15.0 equiv) in DCM (10 mL) at 0° C. The mixture was stirred at 25° C. for 1 h. The mixture was diluted with water (10 mL) and extracted with DCM (3×10 mL). The combined organic layers were washed with brine (10 mL), dried over sodium sulfate, filtered and concentrated. The residue was purified by prep-HPLC (column: Unisil 3-100 C18 Ultra 150*50 mm*3 m; mobile phase: [water(FA)-ACN]; B%: 28%-58%, 10 min) to give 2-trimethylsilylethyl (2S)-4-[7-(8-chloro-1-naphthyl)-2-[[(2S)-1-[3-[2-[2-[2-1[2-1[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]propyl]pyrrolidin-2-yl]methoxy]-6,8-dihydro-5H-pyrido[3,4-d]pyrimidin-4-yl]-2-(cyanomethyl)piperazine-1-carboxylate (30 mg, 18% yield) was obtained as a brown solid.

Step 8. To a solution of 2-trimethylsilylethyl (2S)-4-[7-(8-chloro-1-naphthyl)-2-[[(2S)-1-[3-[-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]propyl]pyrrolidin-2-yl]methoxy]-6,8-dihydro-SH-pyrido[3,4-d]pyrimidin-4-yl]-2-(cyanomethyl)piperazine-1-carboxylate (30 mg, 23 μmol, 1.0 equiv) in DCM (1 mL) was added TFA (0.2 mL). The mixture was stirred at 25° C. for 1 h and concentrated under reduced pressure to give N-[2-[2-[2-[2-[3-[(2S)-2-1[[7-(8-chloro-1-naphthyl)-4-[(3S)-3-(cyanomethyl)piperazin-1-yl]-6,8-dihydro-SH-pyrido[3,4-d]pyrimidin-2-yl]oxymethyl]pyrrolidin-1-yl]propoxy]ethoxy]ethoxy]ethoxy]ethyl]-4-[[(7R)-8-cyclopentyl-7-ethyl-5-methyl-6-oxo-7H-pteridin-2-yl]amino]-3-methoxy-benzamide (26 mg, TFA salt) as a colorless oil without purification for the next step.

Step 9. To a solution of N-[2-[2-[2-[2-[3-[(2S)-2-[[7-(8-chloro-1-naphthyl)-4-[(3S)-3-(cyanomethyl)piperazin-1-yl]-6,8-dihydro-5H-pyrido [3,4-d]pyrimidin-2-yl]oxymethyl]pyrrolidin-1-yl]propoxy]ethoxy]ethoxy]ethoxy]ethyl]-4-[[(7R)-8-cyclopentyl-7-ethyl-5-methyl-6-oxo-7H-pteridin-2-yl]amino]-3-methoxy-benzamide (26 mg, 23 μmol, 1.0 equiv, TFA salt) and 2-fluoroprop-2-enoic acid (4.1 mg, 46 μmol, 2.0 equiv) in DMF (1 mL) was added DIEA (12 mg, 90 μmol, 4.0 equiv) and HATU (9.4 mg, 25 μmol, 1.1 equiv). The residue was purified by prep-HPLC (column: Unisil 3-100 C18 Ultra 150*50 mm*3 m; mobile phase: [water(FA)-ACN]; B%: 15%-45%, 10 min) to give N-[2-[2-[2-[2-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]propoxy]ethoxy]ethoxy]ethoxy]ethyl]-4-[[(7R)-8-cyclopentyl-7-ethyl-5-methyl-6-oxo-7H-pteridin-2-yl]amino]-3-methoxy-benzamide (8.7 mg, 26% yield) as a white solid. ¹H NMR (400 MHz, CDCl₃): δ8.53 (d, J=8.4 Hz, 1H), 8.02 (s, 1H), 7.75 (d, J=8.2 Hz, 1H), 7.67 (s, 1H), 7.65-7.56 (m, 2H), 7.51 (d, J=7.4 Hz, 1H), 7.48-7.42 (m, 2H), 7.39 (d, J=9.6 Hz, 1H), 7.33 (t, J=7.8 Hz, 1H), 7.26-7.18 (m, 1H), 7.12 (s, 1H), 5.48-5.32 (m, 1H), 5.24 (d, J=3.6 Hz, 1H), 4.53-4.45 (m, 1H), 4.43-4.33 (m, 2H), 4.22 (d, J=3.6 Hz, 1H), 4.16-4.10 (m, 1H), 4.05 (d, J=13.2 Hz, 1H), 3.96 (s, 3H), 3.75 (s, 1H), 3.73 (s, 2H), 3.71-3.69 (m, 2H), 3.63 (d, J=13.6 Hz, 6H), 3.60-3.52 (m, 12H), 3.32 (s, 3H), 3.23-3.12 (m, 4H), 2.96 (s, 2H), 2.88 (s, 2H), 2.63-2.54 (m, 2H), 2.17-2.07 (m, 4H), 1.97 (s, 2H), 1.75-1.67 (m, 4H), 1.24 (t, J=7.2 Hz, 8H), 0.87 (t, J=7.6 Hz, 3H). LC-MS: MS (ES′): RT=2.050 min, m/z=1231.0 [M+H⁺]; LCMS method: 25

### Example 90—Synthesis of Compound I-106

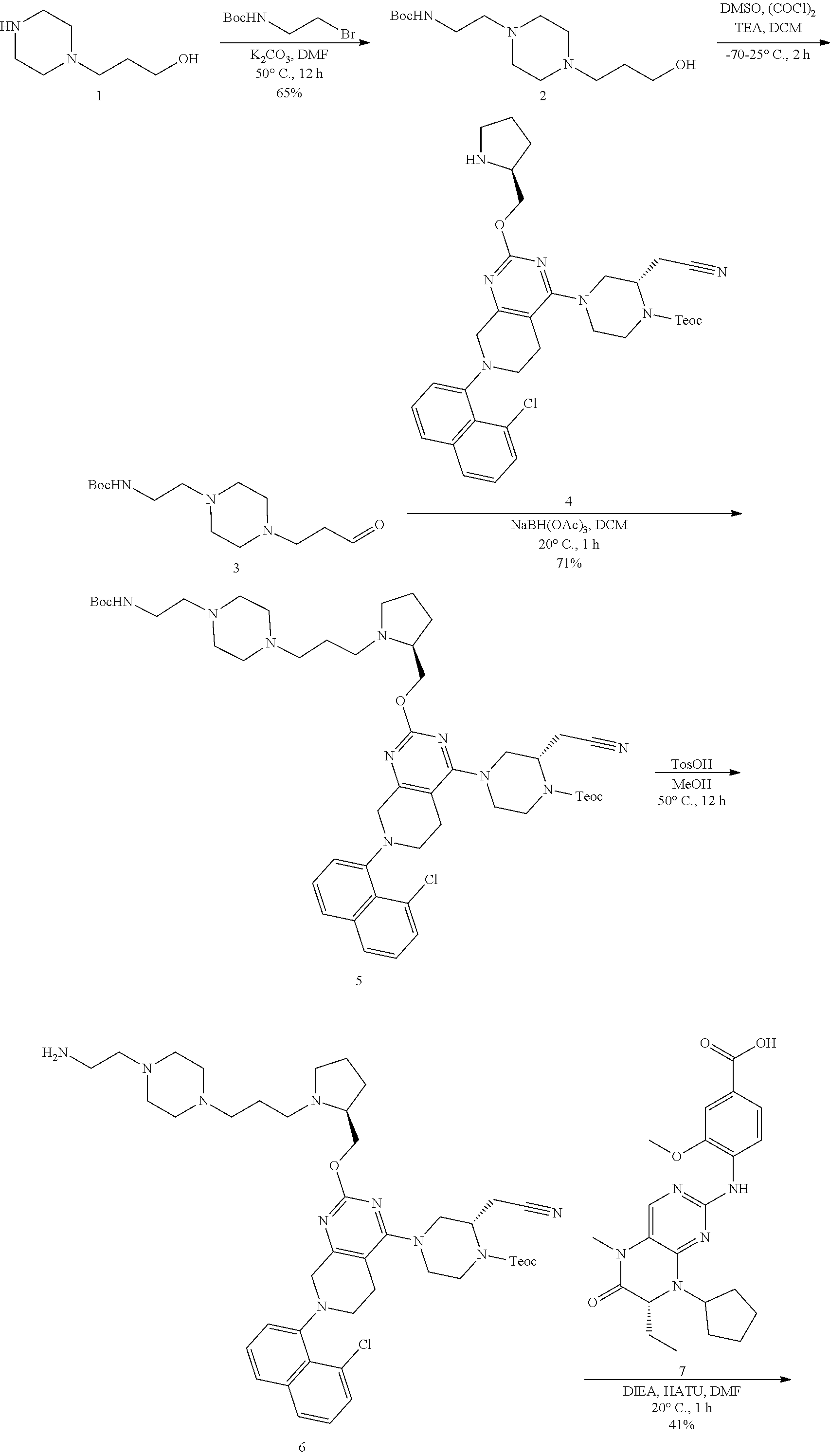

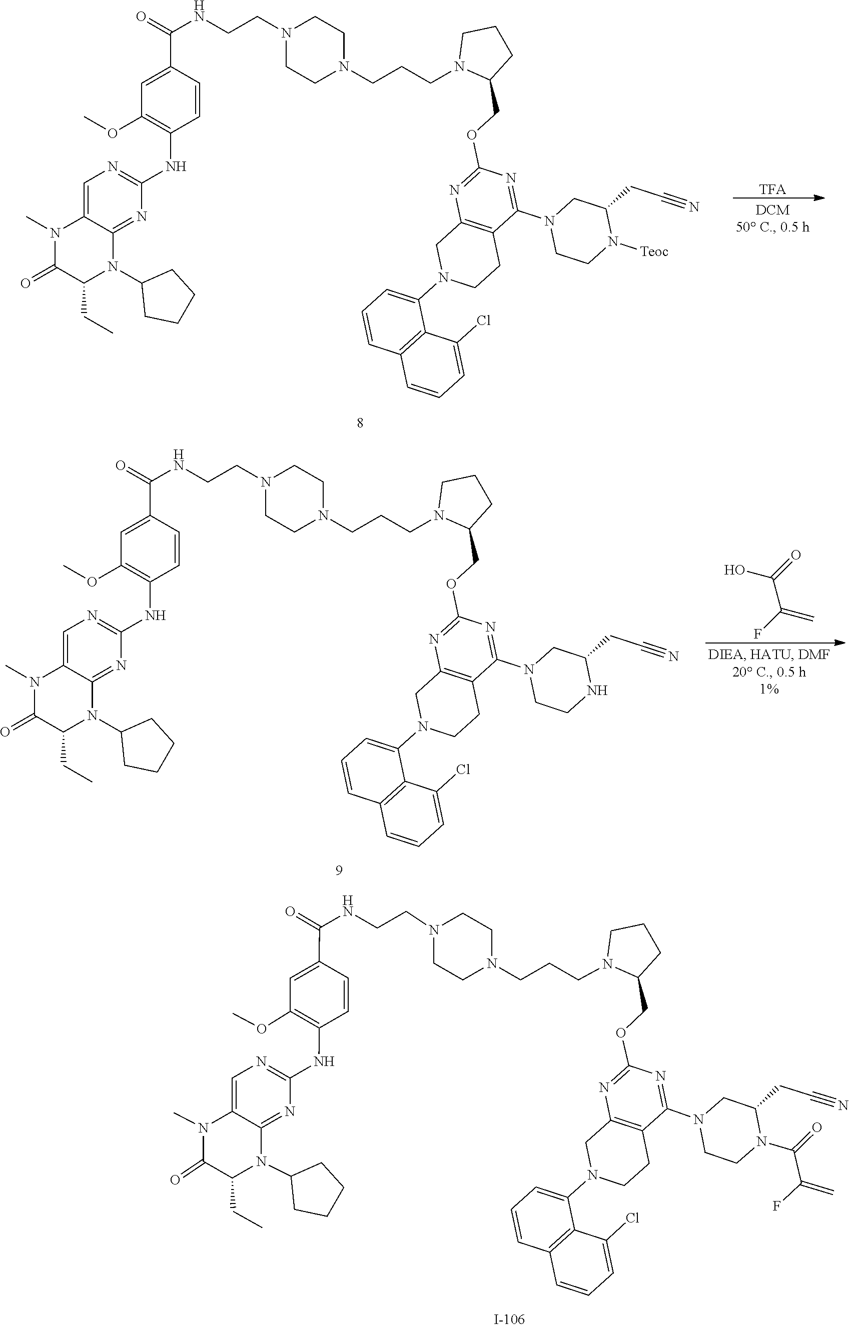

Step 1. The solution of 3-piperazin-1-ylpropan-1-ol (1 g, 6.9 mmol, 1.0 equiv) and tert-butyl N-(2-bromoethyl)carbamate (1.7 g, 7.6 mmol, 1.1 equiv) and K₂CO₃ (2.88 g, 20.8 mmol, 3.0 equiv) in DMF (5 mL) was stirred at 50° C. for 12 h. The mixture was filtered to get the filtrate, which was purified by prep-HPLC (column: Waters Xbridge 150*25 mm*5 m; mobile phase: [water(NH₄HCO₃)-ACN]; B%: 5%-35%, 10 min) to give tert-butyl N-[2-[4-(3-hydroxypropyl)piperazin-1-yl]ethyl]carbamate (1.3 g, 65% yield) as brown oil. ¹H NMR (400 MHz, CDCl₃): δ 4.96-4.79 (m, 1H), 4.58-4.56 (m, 1H), 3.80-3.67 (m, 2H), 3.23-3.05 (m, 2H), 2.72-2.11 (m, 12H), 1.66-1.62 (m, 2H), 1.38 (s, 10H).

Step 2. To a solution of DMSO (489 mg, 6.26 mmol, 489 μL, 10.0 equiv) in DCM (3 mL) was added (COCl)₂ (319 mg, 2.5 mmol, 0.2 mL, 4.0 equiv) in DCM (3 mL) at −70° C. and stirred for 0.5 h. Then tert-butyl N-[2-[4-(3-hydroxypropyl)piperazin-1-yl]ethyl]carbamate (180 mg, 626 μmol, 1.0 equiv) in DCM (1 mL) was added and stirred at −70° C. for 0.5 h. Then TEA (950 mg, 9.39 mmol, 1.3 mL, 15.0 equiv) in DCM (1 mL) was added to the solution and stirred at −70° C. for 0.5 h. The mixture was slowly warmed to 25° C. and stirred at 25° C. for 0.5 h. The solution was used for next step directly.

Step 3. To the solution of tert-butyl N-[2-[4-(3-oxopropyl)piperazin-1-yl]ethyl]carbamate (150 mg, 525 μmol, 2.0 equiv) in DCM (3 mL) was added 2-trimethylsilylethyl (2S)-4-[7-(8-chloro-1-naphthyl)-2-[[(2S)-pyrrolidin-2-yl]methoxy]-6,8-dihydro-5H-pyrido[3,4-d]pyrimidin-4-yl]-2-(cyanomethyl)piperazine-1-carboxylate (200 mg, 257 μmol, 1.0 equiv, TFA salt) and NaBH(OAc)₃ (1.09 g, 5.15 mmol, 20.0 equiv), then the solution was stirred at 20° C. for 1 h. The mixture was concentrated and purified by prep-HPLC (column: Phenomenex luna C18 150*40 mm*15 m; mobile phase: [water(FA)-ACN]; B%: 22%-52%, 10 min) to give 2-trimethylsilylethyl (2S)-4-[2-[[(2S)-1-[3-[4-[2-(tert-butoxycarbonylamino)ethyl]piperazin-1-yl]propyl]pyrrolidin-2-yl]methoxy]-7-(8-chloro-1-naphthyl)-6,8-dihydro-SH-pyrido[3,4-d]pyrimidin-4-yl]-2-(cyanomethyl)piperazine-1-carboxylate (170 mg, 71% yield). ¹H NMR (400 MHz, CDCl₃): δ7.74-7.66 (m, 1H), 7.60-7.53 (m, 1H), 7.50-7.33 (m, 2H), 7.30-7.25 (m, 1H), 7.19-7.11 (m, 1H), 5.13-4.92 (m, 1H), 4.64-3.70 (m, 16H), 3.58-2.89 (m, 12H), 2.81-2.34 (m, 16H), 1.38 (s, 9H), 1.05-0.87 (m, 2H), 0.00 (s, 9H).

Step 4. The solution of 2-trimethylsilylethyl (2S)-4-[2-[[(2S)-1-[3-[4-[2-(tert-butoxycarbonylamino)ethyl]piperazin-1-yl]propyl]pyrrolidin-2-yl]methoxy]-7-(8-chloro-1-naphthyl)-6,8-dihydro-SH-pyrido[3,4-d]pyrimidin-4-yl]-2-(cyanomethyl)piperazine-1-carboxylate (170 mg, 182 μmol, 1.0 equiv) and TosOH (157 mg, 912 μmol, 5.0 equiv) in MeOH (3 mL) was stirred at 50° C. for 12 h. The solution was concentrated under reduced pressure to give 2-trimethylsilylethyl (2S)-4-[2-[[(2S)-1-[3-[4-(2-aminoethyl)piperazin-1-yl]propyl]pyrrolidin-2-yl]methoxy]-7-(8-chloro-1-naphthyl)-6,8-dihydro-H-pyrido[3,4-d]pyrimidin-4-yl]-2-(cyanomethyl)piperazine-1-carboxylate (150 mg, crude) as brown oil.

Step 5. To the solution of 2-trimethylsilylethyl (2S)-4-[2-[[(2S)-1-[3-[4-(2-aminoethyl)piperazin-1-yl]propyl]pyrrolidin-2-yl]methoxy]-7-(8-chloro-1-naphthyl)-6,8-dihydro-5H-pyrido[3,4-d]pyrimidin-4-yl]-2-(cyanomethyl)piperazine-1-carboxylate (120 mg, 144 μmol, 1.0 equiv) and 4-[ [(7R)-8-cyclopentyl-7-ethyl-5-methyl-6-oxo-7H-pteridin-2-yl]amino]-3-methoxy-benzoic acid (74 mg, 174 μmol, 1.2 equiv) in DMF (1 mL) was added DIEA (148 mg, 1.1 mmol, 8.0 equiv) and HATU (82.3 mg, 216 μmol, 1.5 equiv), the solution was stirred at 20° C. for 1 h. The solution was concentrated and purified by prep-TLC(SiO₂, Ethyl acetate: Methanol=10:1) to give 2-trimethylsilylethyl (2S)-4-[7-(8-chloro-1-naphthyl)-2-[[(2S)-1-[3-[4-[2-[[4-[[(7R)-8-cyclopentyl-7-ethyl-5-methyl-6-oxo-7H-pteridin-2-yl]amino]-3-methoxy-benzoyl]amino]ethyl]piperazin-1-yl]propyl]pyrrolidin-2-yl]methoxy]-6,8-dihydro-5H-pyrido[3,4-d]pyrimidin-4-yl]-2-(cyanomethyl)piperazine-1-carboxylate (74 mg, 41% yield).

Step 6. The solution of 2-trimethylsilylethyl (2S)-4-[7-(8-chloro-1-naphthyl)-2-[[(2S)-1-[3-[4-[2-[[4-[[(7R)-8-cyclopentyl-7-ethyl-5-methyl-6-oxo-7H-pteridin-2-yl]amino]-3-methoxy-benzoyl]amino]ethyl]piperazin-1-yl]propyl]pyrrolidin-2-yl]methoxy]-6,8-dihydro-5H-pyrido[3,4-d]pyrimidin-4-yl]-2-(cyanomethyl)piperazine-1-carboxylate (74 mg, 60 μmol, 1.0 equiv) in TFA (1 mL) and DCM (10 mL) was stirred at 20° C. for 0.5 h. The mixture was concentrated to give N-[2-[4-[3-[(2S)-2-[[7-(8-chloro-1-naphthyl)-4-[(3S)-3-(cyanomethyl)piperazin-1-yl]-6,8-dihydro-5H-pyrido[3,4-d]pyrimidin-2-yl]oxymethyl]pyrrolidin-1-yl]propyl]piperazin-1-yl]ethyl]-4-[[(7R)-8-cyclopentyl-7-ethyl-5-methyl-6-oxo-7H-pteridin-2-yl]amino]-3-methoxy-benzamide (72 mg, crude, TFA salt).

Step 7. To the solution of N-[2-[4-[3-[(2S)-2-[[7-(8-chloro-1-naphthyl)-4-[(3S)-3-(cyanomethyl)piperazin-1-yl]-6,8-dihydro-5H-pyrido[3,4-d]pyrimidin-2-yl]oxymethyl]pyrrolidin-1-yl]propyl]piperazin-1-yl]ethyl]-4-[[(7R)-8-cyclopentyl-7-ethyl-5-methyl-6-oxo-7H-pteridin-2-yl]amino]-3-methoxy-benzamide (72 mg, 60 μmol, 1.0 equiv, TFA salt) and 2-fluoroprop-2-enoic acid (10 mg, 119 μmol, 2.0 equiv) in DMF (0.5 mL) was added DIEA (38 mg, 298 μmol, 5.0 equiv) and HATU (34 mg, 89 μmol, 1.5 equiv), then the solution was stirred at 20° C. for 0.5 h. The mixture was filtered and purified by prep-HPLC (column: Phenomenex C18 75*30 mm*3 m; mobile phase: [water(FA)-ACN]; B%: 15%-45%, 7 min) to give N-[2-[4-[3-[(2S)-2-[[7-(8-chloro-1-naphthyl)-4-[(3S)-3-(cyanomethyl)-4-(2-fluoroprop-2-enoyl)piperazin-1-yl]-6,8-dihydro-5H-pyrido[3,4-d]pyrimidin-2-yl]oxymethyl]pyrrolidin-1-yl]propyl]piperazin-1-yl]ethyl]-4-[[(7R)-8-cyclopentyl-7-ethyl-5-methyl-6-oxo-7H-pteridin-2-yl]amino]-3-methoxy-benzamide (1.11 mg, 1% yield). ¹H NMR (400 MHz, MeOD): δ8.61-8.44 (m, 1H), 8.36-8.27 (m, 2H), 7.87-7.78 (m, 2H), 7.73-7.64 (m, 1H), 7.55-7.28 (m, 5H), 5.37-5.28 (m, 2H), 4.57-3.47 (m, 12H), 3.41-3.35 (m, 9H), 3.27-3.12 (m, 8H), 3.01-2.64 (m, 12H), 2.42-2.28 (m, 1H), 2.41-2.09 (m, 2H), 2.03-1.65 (m, 12H), 1.42-1.14 (m, 3H), 0.94-0.81 (m, 3H). LC-MS: MS (ES′): RT=1.810 min, m/z=1166.6 [M+H⁺]; LCMS method: 25

### Example 91—Synthesis of Compound I-107

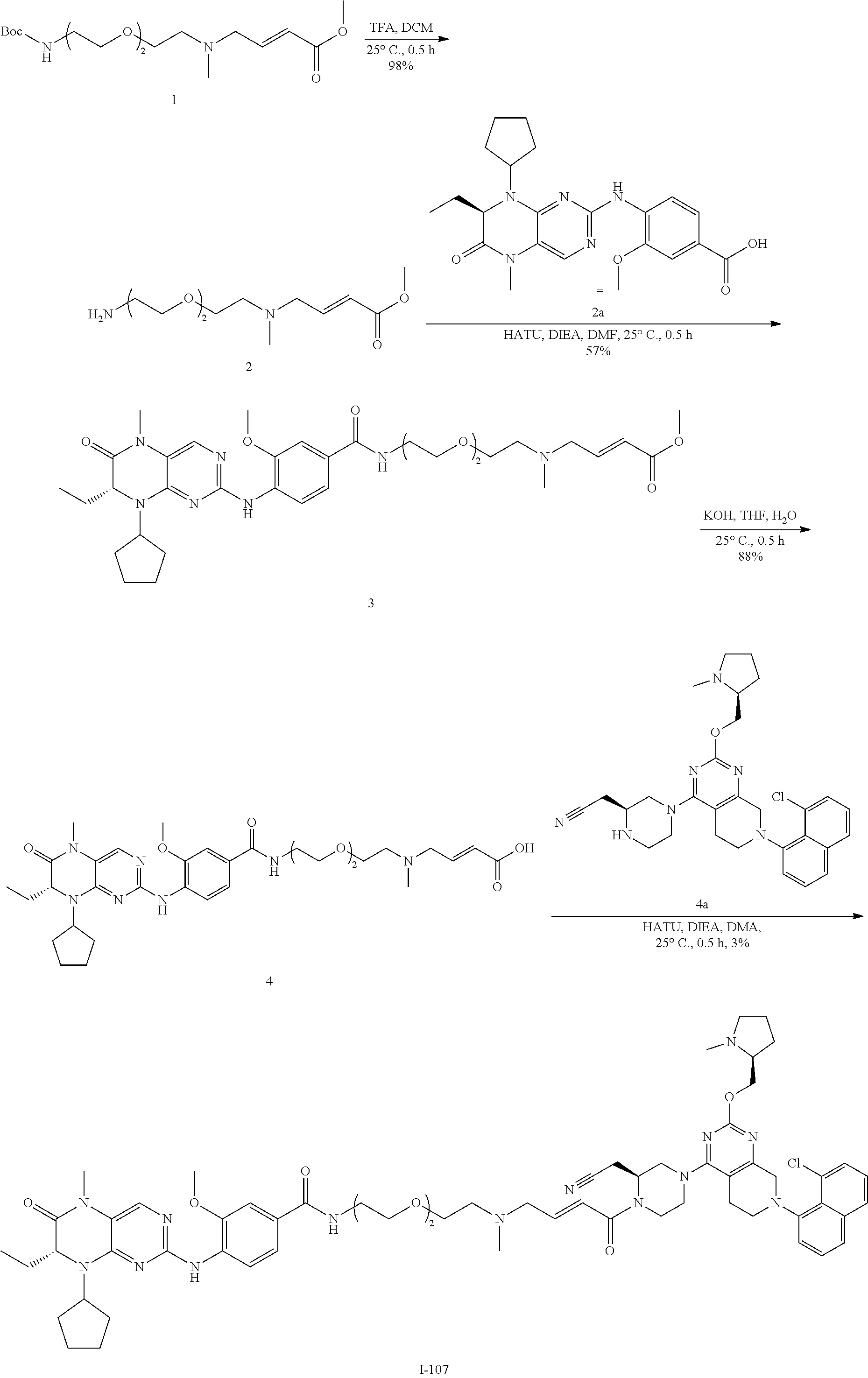

General Information: Compound 4a is a known compound from WO2021/108683, 2021, A1

Step 1. To a solution of methyl (E)-4-[2-[2-[2-(tert-butoxycarbonylamino) ethoxy]ethoxy]ethyl-methyl-amino]but-2-enoate (430 mg, 1.19 mmol, 1 equiv) in DCM (2 mL) was added TFA (1 mL). The mixture was stirred at 25° C. for 0.5 h and concentrated to give methyl (E)-4-[2-[2-(2-aminoethoxy)ethoxy]ethyl-methyl-amino]but-2-enoate (440 mg, 98% yield).

Step 2. To a solution of methyl (E)-4-[2-[2-(2-aminoethoxy)ethoxy]ethyl-methyl-amino]but-2-enoate (440 mg, 1.18 mmol, 1 equiv, TFA) in DMF (3 mL) was added 4-[[(7R)-8-cyclopentyl-7-ethyl-5-methyl-6-oxo-7H-pteridin-2-yl]amino]-3-methoxy-benzoic acid (500 mg, 1.18 mmol, 1 equiv), HATU (536 mg, 1.41 mmol, 1.2 equiv) and DIEA (759 mg, 5.88 mmol, 5 equiv). The mixture was stirred at 25° C. for 0.5 h. The reaction mixture was concentrated. The residue was purified by prep-HPLC (column: Waters Xbridge C₁₈ 150*50 mm*10 m; mobile phase: [water (NH₄HCO₃)-ACN]; B%: 37%-67%, 11 min) to give methyl (E)-4-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethyl-methyl-amino]but-2-enoate (450 mg, 57% yield) as a yellow oil.

Step 3. To a solution of methyl (E)-4-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethyl-methyl-amino]but-2-enoate (450 mg, 673 μmol, 1 equiv) in THF (1 mL) and H₂O (1 mL) was added KOH (113 mg, 2.02 mmol, 3 equiv). The mixture was stirred at 25° C. for 0.5 h. The reaction mixture was concentrated under reduced pressure to give a residue. The residue was purified by prep-HPLC (column: Waters Xbridge C18 150*50 mm*10 m; mobile phase: [water (NH₄HCO₃)-ACN]; B%: 17%-47%, 11 min) to give (E)-4-[2-[2-[2-[[4-[[(7R)-8-cyclopentyl-7-ethyl-5-methyl-6-oxo-7H-piperidin-2-yl]amino]-3-methoxy-benzoyl]amino]ethoxy]ethoxy]ethyl-methyl-amino]but-2-enoic acid (388 mg, 88% yield). ¹H NMR (400 MHz, METHANOL-d4): δ8.52 (d, J=8.4 Hz, 1H), 7.80 (s, 1H), 7.54-7.48 (m, 2H), 6.61-6.52 (m, 1H), 6.18 (d, J=15.6 Hz, 1H), 4.57-4.47 (m, 1H), 4.35-4.29 (m, 1H), 4.02 (s, 3H), 3.78 (t, J=5.2 Hz, 2H), 3.73-3.68 (m, 8H), 3.63-3.58 (m, 2H), 3.35 (s, 3H), 3.14 (t, J=5.0 Hz, 2H), 2.71 (s, 3H), 2.22-2.14 (m, 1H), 2.07-1.85 (m, 6H), 1.81-1.71 (m, 3H), 0.88 (t, J=7.5 Hz, 3H)

Step 4. To a solution of (E)-4-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethyl-methyl-amino]but-2-enoic acid (50 mg, 76 μmol, 1 equiv) in DMA (1 mL) was added HATU (34 mg, 91 μmol, 1.2 equiv) 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]piperazin-2-yl]acetonitrile (40 mg, 76 μmol, 1 equiv) and DIEA (49 mg, 382 μmol, 5 equiv). The mixture was stirred at 25° C. for 0.5 h and concentrated. The residue was purified by prep-HPLC (column: Unisil 3-100 Cis Ultra 150*50 mm*3 m; mobile phase: [water (FA)-ACN]; B%: 10%-30%, 10 min) to give N-[2-[2-[2-[[(E)-4-[(2S)-4-[7-(8-chloro-1-naphthyl)-2-[[(2S)-1-methylpyrrolidin-2-yl]methoxy]-6,8-dihydro-SH-pyrido[3,4-d]pyrimidin-4-yl]-2-(cyanomethyl)piperazin-1-yl]-4-oxo-but-2-enyl]-methyl-amino]ethoxy]ethoxy]ethyl]-4-[[(7R)-8-cyclopentyl-7-ethyl-5-methyl-6-oxo-7H-pteridin-2-yl]amino]-3-methoxy-benzamide (2 mg, 3% yield). ¹H NMR (400 MHz, METHANOL-d4) δ 8.60-8.45 (m, 3H), 7.91-7.63 (m, 2H), 7.55-7.45 (m, 4H), 7.41-7.36 (m, 1H), 7.31-7.24 (m, 1H), 6.89-6.78 (m, 1H), 4.67-4.42 (m, 11H), 4.36-4.18 (m, 3H), 4.07-3.91 (m, 4H), 3.81-3.42 (m, 16H), 3.25-2.99 (m, 5H), 2.95-2.77 (m, 5H), 2.75-2.58 (m, 3H), 2.39-2.35 (m, 3H), 2.06-1.65 (m, 13H), 1.33-1.30 (m, 1H), 0.84 (t, J=7.6 Hz, 3H). LC-MS: MS (ES′): RT=1.694 min, m/z=584.3 [M/2+H⁺]; LCMS method: 25

### Example 92—Synthesis of Compound I-108

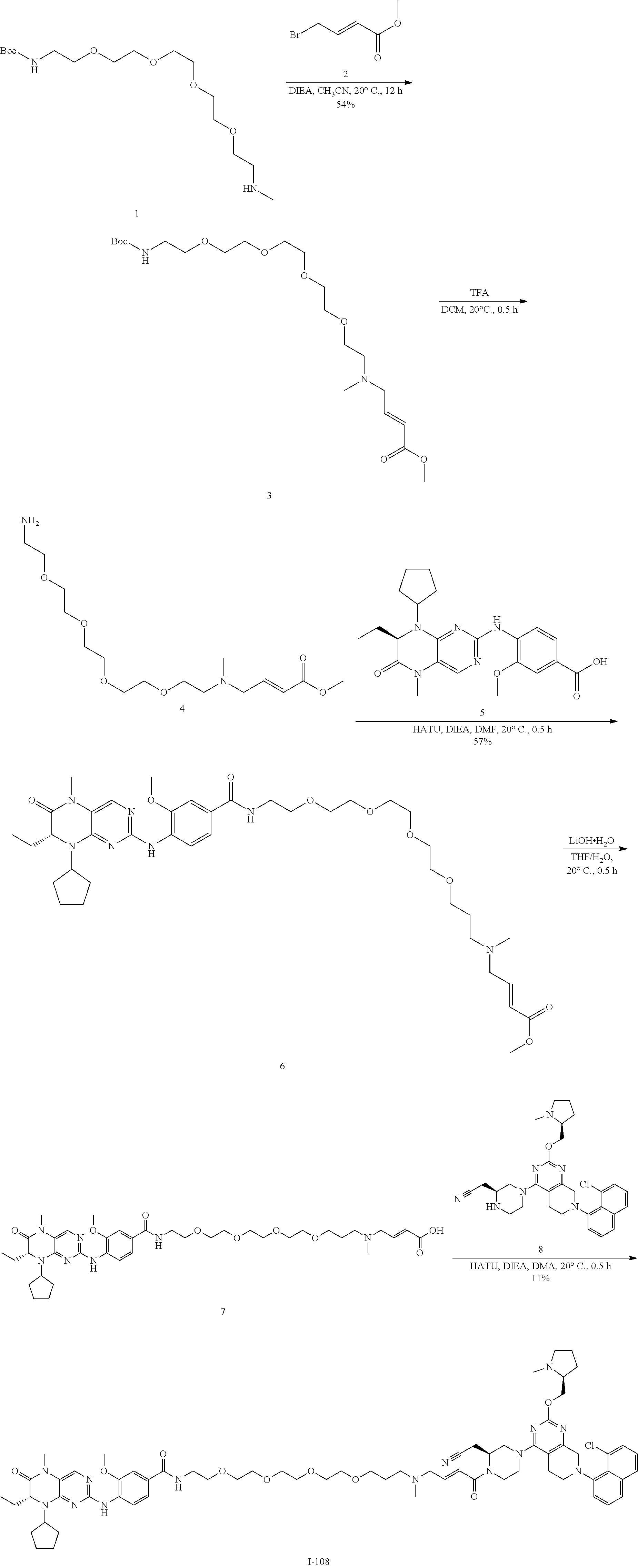

General Information: Compound 5 is a known compound from WO2017/24317, 2017, A2.

Step 1. To a solution of compound 1 (400 mg, 888 μmol, 1.0 equiv), compound 2 (175 mg, 977 μmol, 115 μL, 1.1 equiv) in CH₃CN (3.5 mL) was added DIEA (344 mg, 2.66 mmol, 464 μL, 3.0 equiv). The mixture was stirred at 20° C. for 12 h. The reaction mixture was concentrated under reduced pressure to give a residue. The residue was purified by prep-HPLC (column: Waters XbridgeC18 150*50 mm*10%m; mobile phase: [water (NH₄HCO₃)-ACN]; B%: 44%-74%, 11 min) to afford compound 3 (225 mg, 339 μmol, 38% yield,) as a yellow oil.

Step 2. To a solution of compound 3 (225 mg, 410 μmol, 1.0 equiv) in DCM (2 mL) was added TFA (46.8 mg, 0.41 mmol, 30 μL, 1.0 equiv). The mixture was stirred at 20° C. for 0.5 h. The reaction mixture was concentrated under reduced pressure to give compound 4 (189 mg, crude, TFA salt) as a yellow oil and used into next step directly.

Step 3. To a solution of compound 4 (189 mg, 409 μmol, 1.0 equiv, TFA salt), compound 5 (209 mg, 490 μmol, 1.2 equiv) in DMF (5 mL) was added HATU (233 mg, 613 μmol, 1.5 equiv) and DIEA (158 mg, 1.23 mmol, 214 μL, 3.0 equiv). The mixture was stirred at 20° C. for 0.5 h. The reaction mixture was quenched by addition H₂O 0.3 mL at 20° C. and concentrated under reduced pressure to remove solvent. The residue was purified by prep-HPLC (column: Phenomenex Synergi Polar-RP 100*25 mm*4 m; mobile phase: [water (TFA)-ACN]; B%: 26%-46%, 7 min) to afford compound 6 (280 mg, 370 μmol, 91% yield) as a yellow oil. ¹H NMR: (400 MHz, CDCl3) δ 7.76 (d, J=8.1 Hz, 1H), 7.50-7.41 (m, 2H), 7.35 (s, 1H), 6.93-6.81 (m, 1H), 6.20 (d, J=15.6 Hz, 1H), 4.35 (d, J=2.9, 6.7 Hz, 1H), 4.18-4.27 (m, 1H), 3.98-3.86 (m, 5H), 3.82-3.73 (m, 5H), 3.72-3.57 (m, 17H), 3.34-3.22 (m, 5H), 2.82 (s, 3H), 2.10-1.97 (m, 3H), 1.91-1.77 (m, 3H), 1.75-1.65 (m, 1H), 1.61 (s, 1H), 1.26 (t, J=7.2 Hz, 1H), 0.87 (t, J=7.4 Hz, 3H)

Step 4. To a solution of compound 6 (280 mg, 370 μmol, 1.0 equiv) in THF (1 mL) was added LiOH.H₂O (1 M, 1 mL, 3.0 equiv) in H₂O (1 mL). The mixture was stirred at 20° C. for 12 h. The reaction mixture was used HCl (1M) to adjusted the pH to 3-4 and concentrated under reduced pressure to give a residue. The residue was purified by prep-HPLC (column: 3_Phenomenex Luna C18 75*30 mm*3 m; mobile phase: [water (TFA)-ACN]; B%: 16%-46%, 7 min) to afford compound 7 (270 mg, 335 μmol, 90% yield) as a yellow solid.

Step 5. To a solution of compound 7 (121 mg, 164 μmol, 1.5 equiv) and compound 8 (58.0 mg, 109 μmol, 1.0 equiv) in DMA (1 mL) was added DIEA (42 mg, 0.33 mmol, 57 μL, 3.0 equiv) and HATU (62.0 mg, 165 μmol, 1.5 equiv). The mixture was stirred at 20° C. for 0.5 h. The reaction mixture was concentrated under reduced pressure to give a residue. The residue was purified by prep-HPLC (column: 3_Phenomenex Luna C18 75*30 mm*3 m; mobile phase:[water(TFA)-ACN]; B%: 25%-55%, 7 min), prep-HPLC (column: Phenomenex luna C18 250*50 mm*15 m; mobile phase: [water(FA)-ACN]; B%: 17%-47%, 10 min) compound I-108 (15 mg, 12 μmol, 11% yield) was obtained as a white solid. LC-MS: MS (ES′): RT=1.841 min, m/z=635.3 [M/2+H]+; LCMS method: 25. ¹H NMR: (400 MHz, CD3OD) δ 7.89-7.80 (m, 2H), 7.73-7.68 (m, 1H), 7.62-7.57 (m, 2H), 7.56-7.47 (m, 3H), 7.39-7.31 (m, 2H), 7.16-6.96 (m, 1H), 6.87-6.76 (m, 1H), 5.14-4.97 (m, 1H), 4.84-4.82 (m, 1H), 4.68-4.58 (m, 2H), 4.47-4.43 (m, 1H), 4.36-4.22 (m, 3H), 4.13-4.03 (m, 2H), 3.97 (d, J=2.2 Hz, 3H), 3.87-3.75 (m, 4H), 3.70-3.64 (m, 14H), 3.62-3.54 (m, 4H), 3.38 (s, 3H), 3.32-3.31 (m, 1H), 3.31-3.29 (m, 2H), 3.28 (d, J=2.1 Hz, 3H), 3.21 (d, J=6.8 Hz, 2H), 3.09-3.01 (m, 4H), 2.98-2.90 (m, 4H), 2.79-2.67 (m, 1H), 2.46-2.35 (m, 1H), 2.28-1.97 (m, 7H), 1.93-1.81 (m, 3H), 1.67-1.52 (m, 4H), 0.83 (t, J=2.9, 7.4 Hz, 3H)

### Example 93—Synthesis of Compound I-109

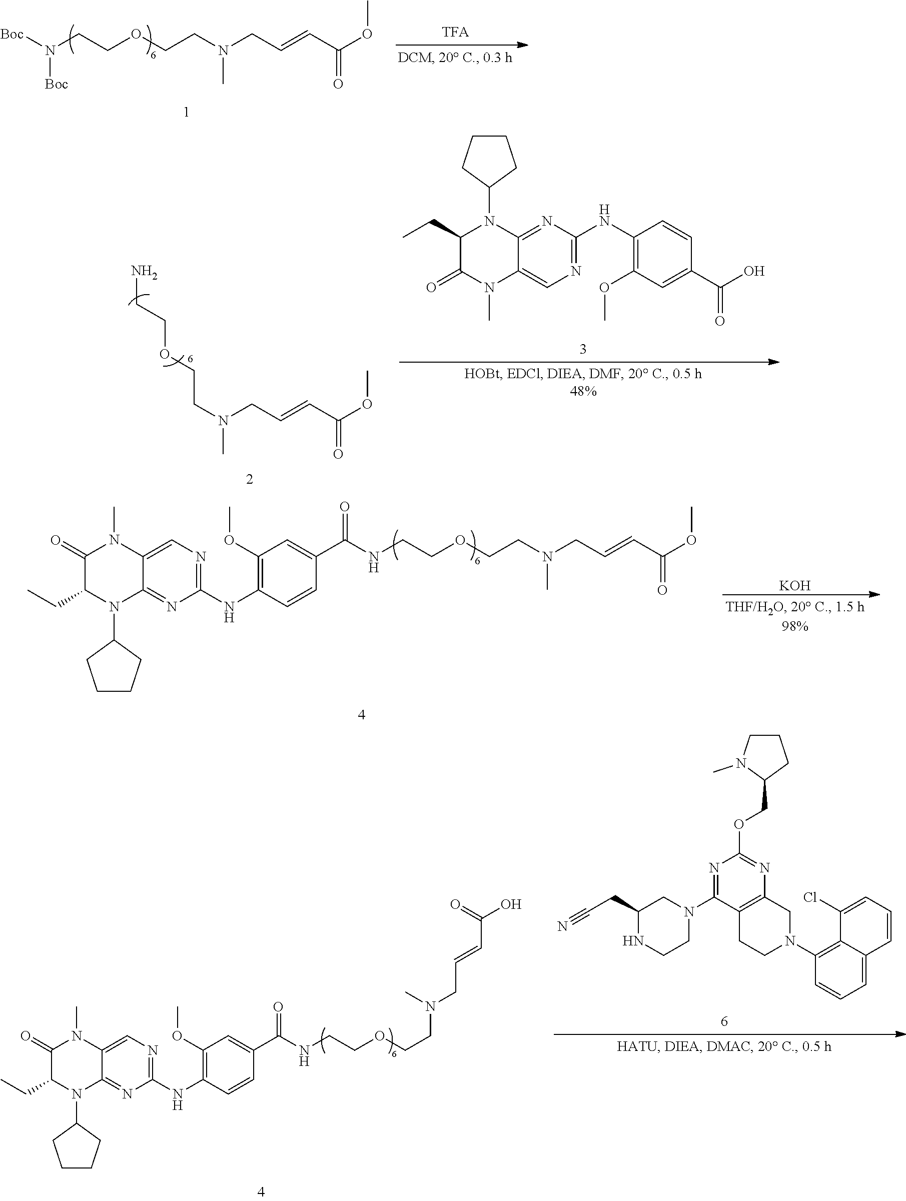

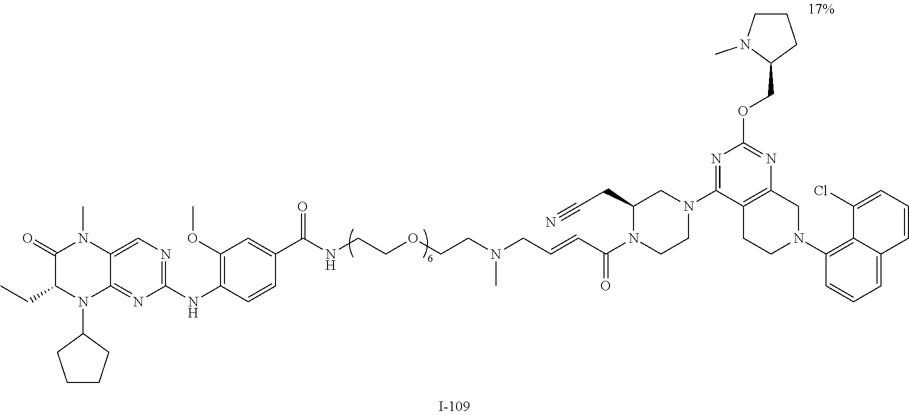

General Information: Compound 3 is a known compound from WO2018/148443. Compound 6 is a known compound from CN113045565

Step 1. To a solution of methyl (E)-4-[2-[2-[2-[2-[2-[2-[2-(tertbutoxycarbonylamino) ethoxy]ethoxy]ethoxy]ethoxy]ethoxy]ethoxy]ethyl-methyl-amino]but-2-enoate (400 mg, 745 μmol, 1.0 equiv) in DCM (5 mL) was added TFA (1 mL), the mixture was stirred at 20° C. for 0.3 h. The reaction mixture was concentrated to give methyl (E)-4-[2-[2-[2-[2-[2-[2-(2-aminoethoxy)ethoxy]ethoxy]ethoxy]ethoxy]ethoxy]ethyl-methyl-amino]but-2-enoate (410 mg, TFA salt) as a yellow oil.

Step 2. To a solution of 4-[[(7R)-8-cyclopentyl-7-ethyl-5-methyl-6-oxo-7H-pteridin-2-yl]amino]-3-methoxy-benzoic acid (316 mg, 744 μmol, 1.0 equiv), methyl (E)-4-[2-[2-[2-[2-[2-[2-(2-aminoethoxy)ethoxy]ethoxy]ethoxy]ethoxy]ethoxy]ethyl-methylamino]but-2-enoate (410 mg, 744 μmol, 1.0 equiv, TFA salt) in DMF (8 mL) was added EDCI (214 mg, 1.12 mmol, 1.5 equiv) and DIEA (288 mg, 2.23 mmol, 3.0 equiv), HOBt (150 mg, 1.12 mmol, 1.5 equiv). The mixture was stirred at 20° C. for 0.5 h. The solution was purified by prep-HPLC (column: Phenomenex luna C18 150*40 mm*15 m; mobile phase: [water(FA)-ACN]; B%: 8%-38%, 10 min) to give methyl (E)-4-[2-[2-[2-[2-[2-[2-[2-[[4-[[(7R)-8-cyclopentyl-7-ethyl-5-methyl-6-oxo-7H-pteridin-2-yl]amino]-3-methoxybenzoyl]amino]ethoxy]ethoxy]ethoxy]ethoxy]ethoxy]ethoxy]ethyl-methyl-amino]but-2-enoate (300 mg, 48% yield).

Step 3. To a solution of methyl (E)-4-[2-[2-[2-[2-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethoxy]ethoxy]ethyl-methyl-amino]but-2-enoate (300 mg, 355 μmol, 1.0 equiv) in THF (5 mL) and H₂O (1 mL) was added KOH (0.5 M, 1.5 mL, 2.0 equiv). The mixture was stirred at 20° C. for 1.5 h. The residue was dissolved in water (20 mL) and the mixture was extracted with EtOAc (30 mL*2). Then the aqueous phase was collected and acidized by 2N HCl to adjust the PH=7, and the mixture was extracted with EtOAc (50 mL×3), the combined organic layers were washed by brine (30 mL), dried over Na₂SO₄, filtered and concentrated to give (E)-4-[22-[2-[2-[2-[[2-[2-[2[4-[ [(7 R)(−78-cyclopentyl-7-ethyl-5-methyl-6-oxo-7H-pteridin-2-yl]amino]-3-methoxybenzoyl]amino]ethoxy]ethoxy]ethoxy]ethoxy]ethoxy]ethoxy]ethyl-methyl-amino]but-2-enoic acid (290 mg, 98% yield).

Step 4. To a solution of 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]piperazin-2-yl]acetonitrile (53 mg, 101 μmol, 1.2 equiv), (E)-4-[2-[2-[2-[2-[2-[2-[2-[[4-[[(7R)-8-cyclopentyl-7-ethyl-5-methyl-6-oxo-7H-pteridin-2-yl]amino]-3-methoxybenzoyl]amino]ethoxy]ethoxy]ethoxy]ethoxy]ethoxy]ethoxy]ethyl-methyl-amino]but-2-enoic acid (70 mg, 84 μmol, 1.0 equiv) in DMAC (3 mL) was added HATU (38 mg, 101 μmol, 1.2 equiv) and DIEA (32 mg, 253 μmol, 3.0 equiv). The mixture was stirred at 20° C. for 0.5h. The solution was purified by prep-HPLC (column: Phenomenex Synergi Polar-RP 100*25 mm*4 m; mobile phase: [water(TFA)-ACN]; B%: 32%-52%, 7 min and column: Waters Xbridge 150*25 mm*5 m; mobile phase: [water(NH₄HCO₃)-ACN]; B%:54%-84%, 8 min) to give N-[2-[2-[2-[2-[2-[2-[2-[[(E)-4-1[(2S)-4-1[7-(8-chloro-1-naphthyl)-2-[[(2S)-1-methylpyrrolidin-2-yl]methoxy]-6,8-dihydro-5Hpyrido1[3,4-d]pyrimidin-4-yl]-2-(cyanomethyl)piperazin-1-yl]-4-oxo-but-2-enyl]-methylamino]ethoxy]ethoxy]ethoxy]ethoxy]ethoxy]ethoxy]ethyl]-4-[[(7R)-8-cyclopentyl-7-ethyl-5-methyl-6-oxo-7H-pteridin-2-yl]amino]-3-methoxy-benzamide (20 mg, 17% yield). ¹H NMR (400 MHz, CD₃OD): δ8.56-8.48 (m, 1H), 7.87-7.75 (m, 2H), 7.73-7.63 (m, 1H), 7.58-7.44 (m, 4H), 7.43-7.35 (m, 1H), 7.32-7.25 (m, 1H), 6.95-6.66 (m, 2H), 4.64 (s, 4H), 4.61-4.58 (m, 1H), 4.55-4.28 (m, 4H), 4.27-3.90 (m, 5H), 3.89-3.36 (m, 32H), 3.26-3.03 (m, 5H), 2.99-2.86 (m, 1H), 2.82-2.58 (m, 4H), 2.55-2.47 (m, 3H), 2.43-2.28 (m, 4H), 2.25-1.65 (m, 15H), 0.89-0.82 (m, 3H). LC-MS: MS (ES⁺): RT=1.776 min, m/z=672.7 [M+H⁺]/2; LCMS method: 25.

### Example 94—Synthesis of Compound I-110

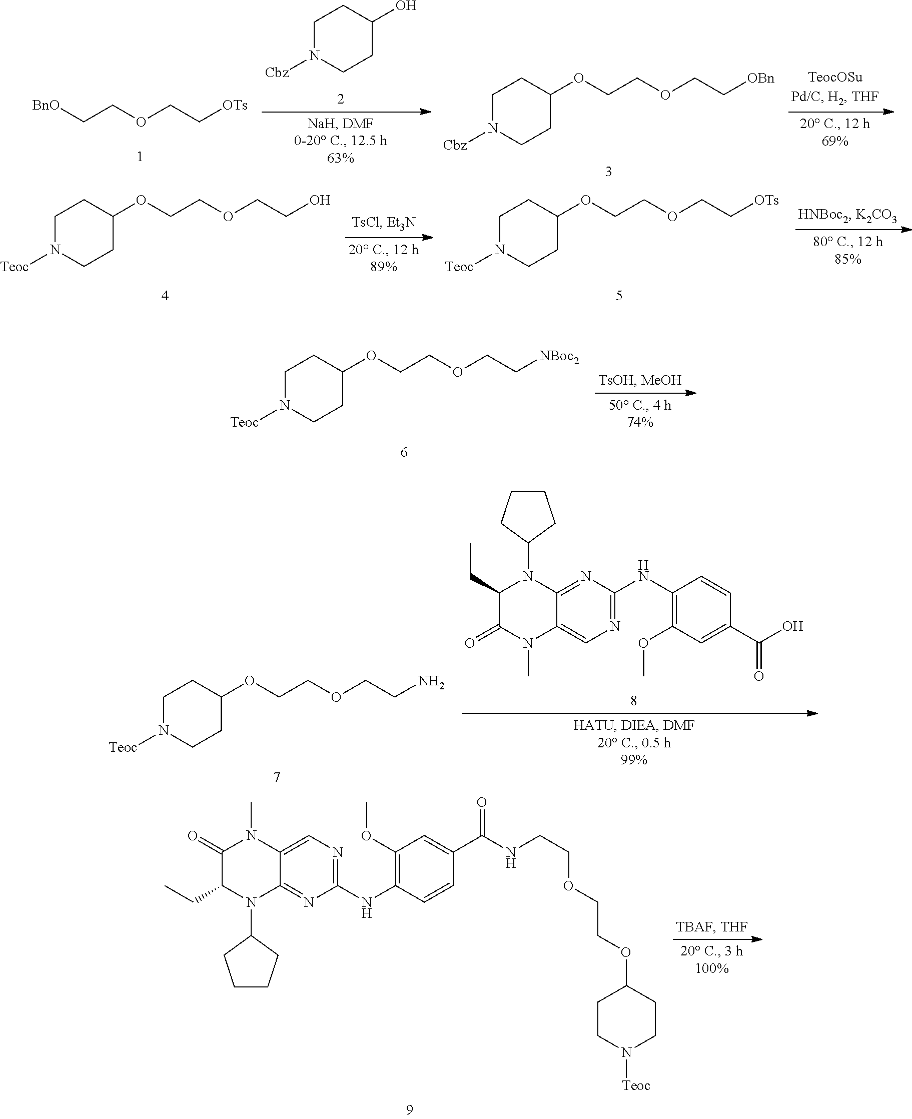

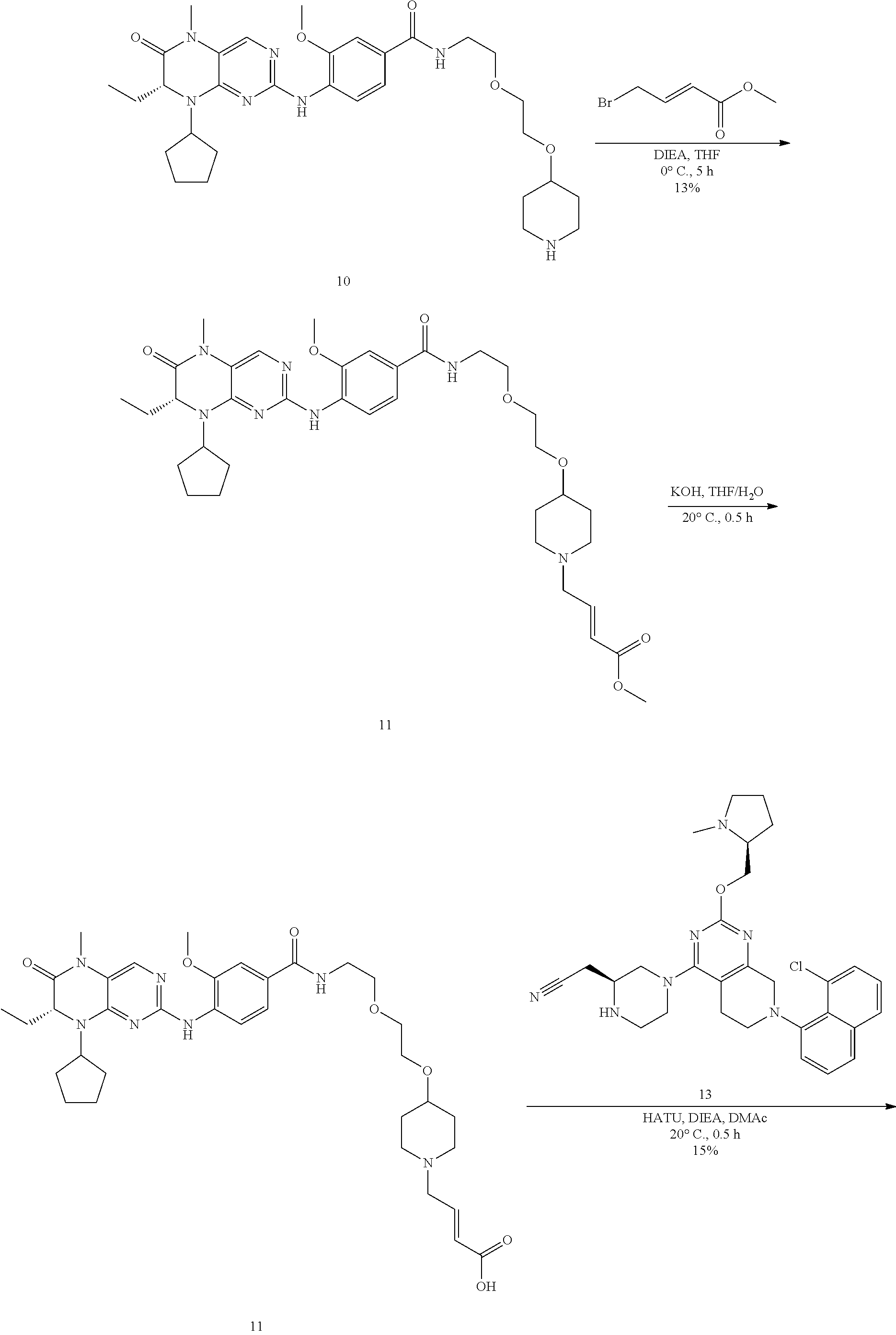

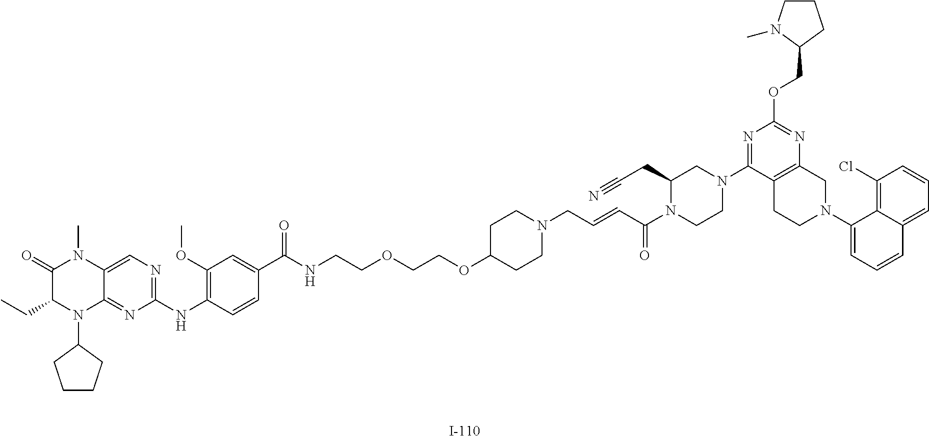 [SMILES: CC[C@@H]1C(=O)N(C)c2cnc(Nc3ccc(C(=O)NCCOCCOC4CCN(C/C=C/C(=O)N5CCN(c6nc(OC[C@@H]7CCCN7C)nc7c6CCN(c6cccc8cccc(Cl)c68)C7)C[C@@H]5CC#N)CC4)cc3OC)nc2N1C1CCCC1]

Step 1. To the solution of benzyl 4-hydroxypiperidine-1-carboxylate (5 g, 21 mmol, 1.0 equiv) in THF (50 mL) was added NaH (1.02 g, 26 mmol, 60% purity, 1.2 equiv) at 0° C., the mixture was stirred at 0° C. for 0.5 h, and 2-(2-benzyloxyethoxy)ethyl 4-methylbenzenesulfonate (7.45 g, 21.2 mmol, 1.0 equiv) was added to the mixture and the mixture was stirred at 20° C. for 12 h. The mixture was diluted with 100 mL H₂O and extracted with EA (100 mL*3). The combined organic layer was concentrated and purified by column chromatography (SiO₂, Petroleum ether/Ethyl acetate=10/1 to 3/1) to give benzyl 4-[2-(2-benzyloxyethoxy)ethoxy]piperidine-1-carboxylate (5.5 g, 63% yield). ¹H NMR (400 MHz, CDCl₃): δ7.29 (s, 10H), 5.15 (s, 2H), 4.59 (s, 2H), 3.95-3.78 (m, 2H), 3.75-3.62 (m, 8H), 3.58-3.49 (m, 1H), 3.26-3.15 (m, 2H), 1.93-1.79 (m, 2H), 1.62-1.49 (m, 2H)

Step 2. To a solution of benzyl 4-[2-(2-benzyloxyethoxy)ethoxy]piperidine-1-carboxylate (10.2 g, 24.7 mmol, 1.0 equiv) and (2,5-dioxopyrrolidin-1-yl) 2-trimethylsilylethyl carbonate (7.04 g, 27.1 mmol, 1.1 equiv) in THF (110 mL) was added Pd/C (2 g, 10% purity) under N₂. The mixture was stirred under H₂ (50 psi) at 20° C. for 12 h. The mixture was filtered and concentrated. The residue was purified by prep-HPLC (column: Waters Xbridge 150*25 mm*5 m; mobile phase: [water(NH₄HCO₃)-ACN]; B%: 30%-60%, 10 min) to give 2-trimethylsilylethyl 4-[2-(2-hydroxyethoxy)ethoxy]piperidine-1-carboxylate (5.7 g, 69% yield). ¹H NMR (400 MHz, CDCl₃): δ 4.25-4.14 (m, 2H), 3.93-3.60 (m, 10H), 3.58-3.46 (m, 1H), 3.23-3.02 (m, 2H), 1.95-1.83 (m, 3H), 1.61-1.50 (m, 2H), 1.11-0.93 (m, 2H), 0.17-0.05 (m, 9H).

Step 3. To the solution of 2-trimethylsilylethyl 4-[2-(2-hydroxyethoxy)ethoxy]piperidine-1-carboxylate (1 g, 3 mmol, 1.0 equiv) and TEA (1.52 g, 15.0 mmol, 2.09 mL, 5.0 equiv) in DCM (5 mL) was added TosCl (1.71 g, 9.0 mmol, 3.0 equiv), then the solution was stirred at 20° C. for 12 h. The solution was filtered and purified by column chromatography (SiO₂, Petroleum ether/Ethyl acetate=10/1 to 3/1) to give 2-trimethylsilylethyl 4-[2-[2-(p-tolylsulfonyloxy)ethoxy]ethoxy]piperidine-1-carboxylate (1.30 g, 89% yield). ¹H NMR (400 MHz, CDCl₃): δ7.76 (d, J=8.4 Hz, 2H), 7.30 (d, J=8.4 Hz, 2H), 4.27-4.10 (m, 4H), 3.80-3.62 (m, 4H), 3.57-3.39 (m, 5H), 3.14-3.03 (m, 2H), 2.41 (s, 3H), 1.84-1.71 (m, 2H), 1.51-1.40 (m, 2H), 1.08-0.88 (m, 2H), 0.00 (s, 9H).

Step 4. The solution of 2-trimethylsilylethyl 4-[2-[2-(p-tolylsulfonyloxy) ethoxy]ethoxy]piperidine-1-carboxylate (1.30 g, 2.67 mmol, 1.0 equiv) and tert-butyl N-tert-butoxycarbonylcarbamate (1.16 g, 5.33 mmol, 2.0 equiv) and K₂CO₃ (1.11 g, 8.0 mmol, 3.0 equiv) in ACN (10 mL) was stirred at 80° C. for 12 h. The mixture was filtered and concentrated. The residue was purified by prep-TLC(SiO₂, Petroleum ether: Ethyl acetate=5:1) to give 2-trimethylsilylethyl 4-[2-1[2-[bis(tert-butoxycarbonyl)amino]ethoxy]ethoxy]piperidine-1-carboxylate (1.20 g, 85% yield).

Step 5. The solution of 2-trimethylsilylethyl 4-[2-[2-[bis(tert-butoxycarbonyl)amino]ethoxy]ethoxy]piperidine-1-carboxylate (0.6 g, 1.13 mmol, 1.0 equiv) and TosOH (213 mg, 1.24 mmol, 1.1 equiv) in MeOH (4 mL) was stirred at 50° C. for 4 h. The solution was filtered and purified by prep-HPLC(column: Waters Xbridge 150*25 mm*5 m; mobile phase: [water(NH₄HCO₃)-ACN]; B%: 15%-45%, 10 min) to give 2-trimethylsilylethyl 4-[2-(2-aminoethoxy)ethoxy]piperidine-1-carboxylate (277 mg, 74% yield). ¹H NMR (400 MHz, CDCl₃): δ 4.19-4.08 (m, 2H), 3.77-3.76 (m, 2H), 3.58 (s, 4H), 3.54-3.41 (m, 3H), 3.08-3.07 (m, 2H), 2.86 (t, J=5.2 Hz, 2H), 1.80-1.79 (m 2H), 1.58-1.43 (m, 2H), 1.03-0.91 (m, 2H), 0.13-0 (s, 9H)

Step 6. To a solution of 2-trimethylsilylethyl 4-[2-(2-aminoethoxy)ethoxy]piperidine-1-carboxylate (327 mg, 983 μmol, 1.0 equiv) in DMF (5 mL) was added HATU (561 mg, 1.48 mmol, 1.5 equiv) and DIEA (381 mg, 2.95 mmol, 3.0 equiv) and 4-[[(7R)-8-cyclopentyl-7-ethyl-5-methyl-6-oxo-7H-pteridin-2-yl]amino]-3-methoxy-benzoic acid (502 mg, 1.18 mmol, 1.2 equiv). The mixture was stirred at 20° C. for 0.5 h. The mixture was diluted with H₂O and extracted with Ethyl acetate 45 mL. The combined organic layers were dried over Na₂SO₄ and concentrated. The residue was purified by column chromatography (SiO₂, Petroleum ether/Ethyl acetate=20/1 to 0/1) to give 2-trimethylsilylethyl 4-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]piperidine-1-carboxylate (717 mg, 99% yield).

Step 7. To a solution of 2-trimethylsilylethyl 4-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]piperidine-1-carboxylate (417 mg, 564 μmol, 1.0 equiv) in THF (5 mL) was added TBAF (1 M, 5 mL, 8.9 equiv). The mixture was stirred at 20° C. for 3 h. The mixture was concentrated and purified by pre-HPLC(column: Waters Xbridge C18 150*50 mm*10 m; mobile phase: [water(NH₄H)-ACN]; B%: 34%-64%, 11 min) to give 4-[[(7R)-8-cyclopentyl-7-ethyl-5-methyl-6-oxo-7H-pteridin-2-yl]amino]-3-methoxy-N-[2-[2-(4-piperidyloxy)ethoxy]ethyl]benzamide (336 mg, 100% yield).

Step 8. To a solution of 4-[[(7R)-8-cyclopentyl-7-ethyl-5-methyl-6-oxo-7H-pteridin-2-yl]amino]-3-methoxy-N-[2-[2-(4-piperidyloxy)ethoxy]ethyl]benzamide (336 mg, 563 μmol, 1.0 equiv) in THF (3 mL) was added DIEA (218 mg, 1.69 mmol, 294 μL, 3.0 equiv) and methyl (E)-4-bromobut-2-enoate (101 mg, 563 μmol, 66 μL, 1.0 equiv). The mixture was stirred at 0° C. for 5 hr. The reaction mixture was diluted with H₂O and extracted with Ethyl acetate 45 mL. The combined organic layers were dried over Na₂SO₄ and concentrated. The residue was purified by prep-TLC (SiO₂, EA: MeOH=20:1) to give methyl (E)-4-[4-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]-1-piperidyl]but-2-enoate (50 mg, 13% yield).

Step 9. To a solution of methyl (E)-4-[4-[2-[2-[ [4-[ [(7R)-8-cyclopentyl-7-ethyl-5-methyl-6-oxo-7H-pteridin-2-yl]amino]-3-methoxy-benzoyl]amino]ethoxy]ethoxy]-1-piperidyl]but-2-enoate (50 mg, 72 μmol, 1.0 equiv) in THF (0.3 mL) was added KOH (20 mg, 360 μmol, 5.0 equiv) and H₂O (0.3 mL).The mixture was stirred at 20° C. for 4 h. The mixture was adjuted the pH to 6-7 with 1M HCl aqueous, then concentrated. The residue was triturated with 50 mL DCM and 5 mL IPA at 25° C. for 0.5 h to give (E)-4-[4-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]-1-piperidyl]but-2-enoic acid (26 mg, 53% yield).

Step 10. To the solution of (E)-4-[4-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]-1-piperidyl]but-2-enoic acid (26 mg, 38 μmol, 1.0 equiv) and 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]piperazin-2-yl]acetonitrile (21 mg, 39 μmol, 1.0 equiv) in DMAC (0.5 mL) was added DIEA (36 mg, 280 μmol, 7.3 equiv) and HATU (19 mg, 50 μmol, 1.3 equiv), then the solution was stirred at 20° C. for 0.5 h. The mixture was purified by prep-HPLC(column: Phenomenex Synergi Polar-RP 100*25 mm*4 m; mobile phase: [water(TFA)-ACN]; B%: 30%-50%, 7 min and column: Waters Xbridge 150*25 mm*5 m; mobile phase: [water(NH₄HCO₃)-ACN]; B%: 68%-98%, 9 min and column: Waters Xbridge 150*25 mm*5 m; mobile phase: [water(NH₄HCO₃)-ACN]; B%: 67%-97%, 9 min) to give N-[2-[2-[[1-[(E)-4-[(2S)-4-[7-(8-chloro-1-naphthyl)-2-[[(2S)-1-methyl pyrrolidin-2-yl]methoxy]-6,8-dihydro-5H-pyrido[3,4-d]pyrimidin-4-yl]-2-(cyanomethyl) piperazin-1-yl]-4-oxo-but-2-enyl]-4-piperidyl]oxy]ethoxy]ethyl]-4-[[(7R)-8-cyclopentyl-7-ethyl-5-methyl-6-oxo-7H-pteridin-2-yl]amino]-3-methoxy-benzamide (7 mg, 15% yield). ¹H NMR (400 MHz, MeOD): δ8.52-8.48 (m, 1H), 7.84-7.82 (m, 1H), 7.78 (s, 1H), 7.69-7.67 (m, 1H), 7.57-7.44 (m, 4H), 7.40-7.28 (m, 3H), 6.88-6.78 (m, 1H), 6.75-6.55 (m, 1H), 4.73-3.88 (m, 14H), 3.79-3.41 (m, 12H), 3.25-3.03 (m, 6H), 2.96-2.59 (m, 5H), 2.52-2.51 (m, 3H), 2.42-1.57 (m, 21H), 1.40-1.21 (m, 3H), 0.93-0.82 (m, 3H). LC-MS: MS (ES⁺): RT=1.702 min, m/z=1193.4 [M+H⁺]; LCMS method: 25.

### Example 95—Synthesis of Compound I-111

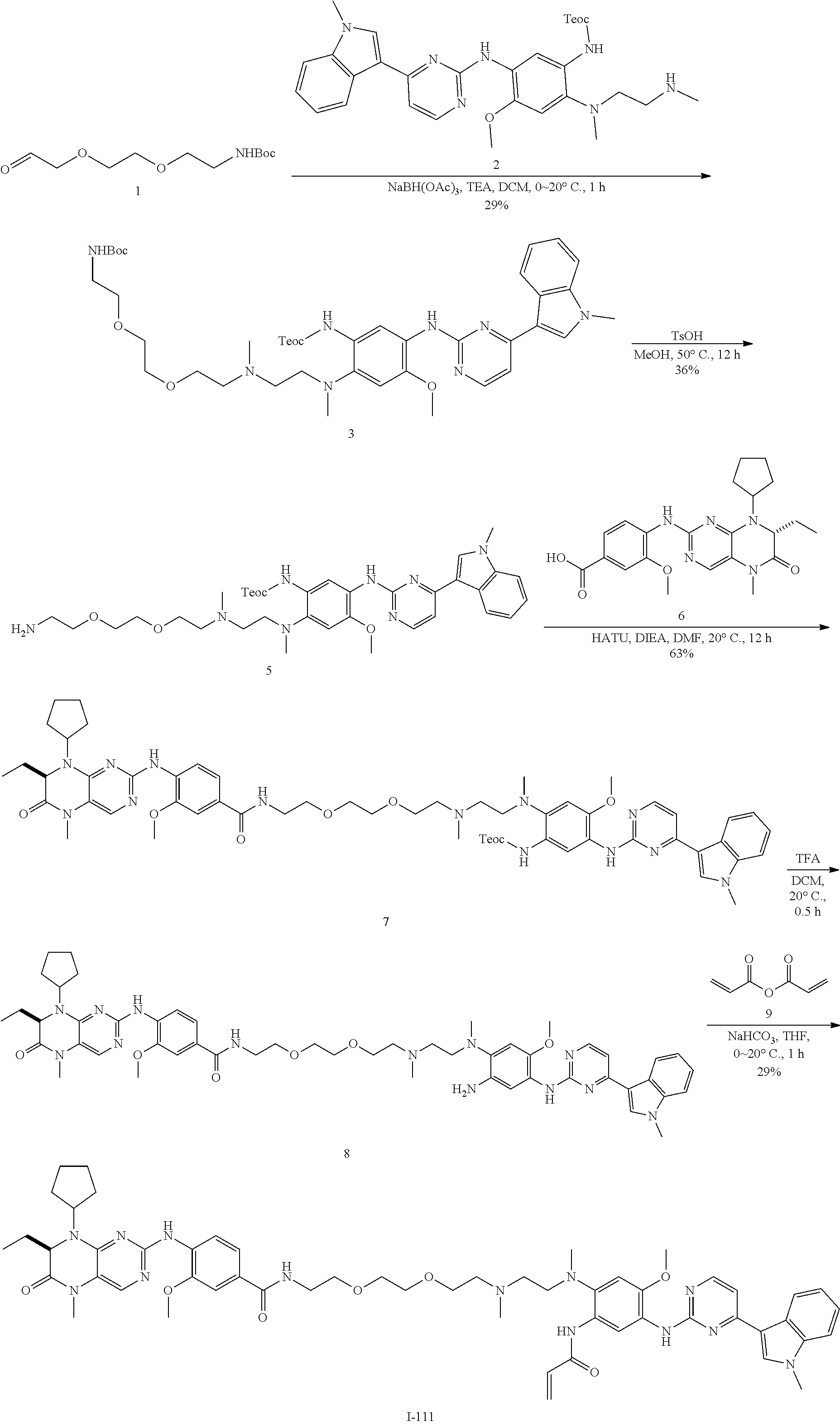

General Information: Compound 1 is a known compound from US2019/192668, 2019, A1. Compound 6 is a known compound from WO2017/24317, 2017, A2.

Step 1. To a solution of compound 1 (215 mg, 868 μmol, 1.0 equiv), NaBH(OAc)₃ (1.84 g, 8.68 mmol, 10 equiv) and TEA (1.05 g, 10.4 mmol, 1.45 mL, 12 equiv) in DCM (5 mL) was added compound 2 (500 mg, 868 μmol, 1.0 equiv) at 0° C. The mixture was stirred at 20° C. for 1 h. The reaction mixture was diluted with NaHCO₃ (50 mL) and extracted with DCM (50 mL×2). The combined organic layers were dried over Na₂SO₄, filtered and concentrated under reduced pressure to give a residue. The residue was purified by prep-HPLC (column: Phenomenex luna C18 150*40 mm*15 m; mobile phase: [water (TFA)-ACN]; B%: 31%-61%, 11 min) to afford compound 3 (202 mg, 250 μmol, 29% yield) as a brown oil. ¹H NMR: (400 MHz, CDCl₃): (8.88-8.72 (m, 1H), 8.13-7.94 (m, 4H), 7.42 (d, J=7.9 Hz, 1H), 7.39-7.32 (m, 1H), 7.21-7.18 (m, 1H), 6.81-6.72 (m, 1H), 4.26-4.18 (m, 2H), 3.96 (s, 2H), 3.90-3.77 (m, 5H), 3.61-3.45 (m, 6H), 3.43-3.13 (m, 8H), 2.96-2.83 (m, 4H), 2.79-2.62 (m, 6H), 1.48-1.40 (m, 9H), 1.28-1.24 (m, 2H), 0.98-0.98 (m, 1H), 0.04 (s, 8H)

Step 2. To a solution of compound 3 (200 mg, 247 μmol, 1.0 equiv) in MeOH (2 mL) was added TsOH (85.0 mg, 496 μmol, 2.0 equiv). The mixture was stirred at 50° C. for 12 h. The reaction mixture was filtered and concentrated under reduced pressure to give a residue. The residue was diluted with DCM 5 mL and used Na₂CO₃ to adjust the pH to 9-10, and was diluted with H₂O 10 mL extracted with DCM 10 mL (5 mL*2). The combined organic layers were dried over Na₂SO₄, filtered and concentrated under reduced pressure to give a residue. The residue was purified by prep-TLC (SiO₂, DCM: MeOH=10:1) to afford compound 5 (73 mg, 89 μmol, 36% yield) as a white solid.

Step 3. To a solution of compound 5 (63 mg, 89 μmol, 1.0 equiv), compound 6 (41 mg, 98 μmol, 1.1 equiv) in DMF (1.5 mL) was added DIEA (34.6 mg, 267 μmol, 47 μL, 3.0 equiv) and HATU (50.8 mg, 134 μmol, 1.5 equiv). The mixture was stirred at 20° C. for 12 h. The reaction mixture was filtered and concentrated under reduced pressure to give a residue. The residue was purified by prep-HPLC (column: 3_Phenomenex Luna C18 75*30 mm*3 m; mobile phase: [water (TFA)-ACN]; B%: 33%-63%, 7 min) to give compound 7 (65 mg, 56 μmol, 63% yield) as a yellow solid.

Step 4. To a solution of compound 7 (65 mg, 58 μmol, 1.0 equiv) in DCM (2.0 mL) was added TFA (1.0 mL). The mixture was stirred at 20° C. for 0.5 h. The reaction mixture was concentrated under reduced pressure to give compound 8 (62 mg, crude, TFA salt) as a yellow solid and used into the next step directly.

Step 5. To a solution of compound 8 (62 mg, 57 μmol, 1.0 equiv, TFA salt) in THF (1 mL) was added NaHCO₃ (216 mg, 2.57 mmol, 44.2 equiv) in H₂O (0.5 mL) was added compound 9 (8.7 mg, 68 μmol, 1.2 equiv) at 0° C. The mixture was stirred at 20° C. for 1 h. The reaction mixture was diluted with water (20 mL) and extracted with EA (10 mL×2). The combined organic layers were dried over Na₂SO₄, filtered and concentrated under reduced pressure to give a residue. The residue was purified by prep-HPLC (column: Phenomenex Synergi C18 150*25 mm*10% m; mobile phase: [water(FA)-ACN]; B%: 10%-40%, 10 min) to give compound I-111 (19 mg, 16 μmol, 29% yield, FA salt) as a yellow solid. LC-MS: MS (ES′): RT=1.601 min, m/z=1024.5 [M+H]+, LCMS method: 25. ¹H NMR: (400 MHz, CD₃OD): (8.86-8.79 (m, 1H), 8.54-8.44 (m, 2H), 8.41 (d, J=8.4 Hz, 1H), 8.30-8.20 (m, 3H), 7.76-7.68 (m, 1H), 7.44-7.35 (m, 3H), 7.24-7.12 (m, 3H), 7.00-6.90 (m, 1H), 6.60-6.37 (m, 2H), 5.88-5.78 (m, 1H), 4.44-4.36 (m, 1H), 4.20 (d, J=2.1 Hz, 1H), 3.99-3.85 (m, 9H), 3.74-3.64 (m, 2H), 3.58-3.46 (m, 8H), 3.28-3.13 (m, 9H), 2.77-2.67 (m, 6H), 2.18-2.07 (m, 1H), 1.88-1.62 (m, 9H), 0.84-0.77 (m, 3H).

### Example 96—Synthesis of Compound I-112

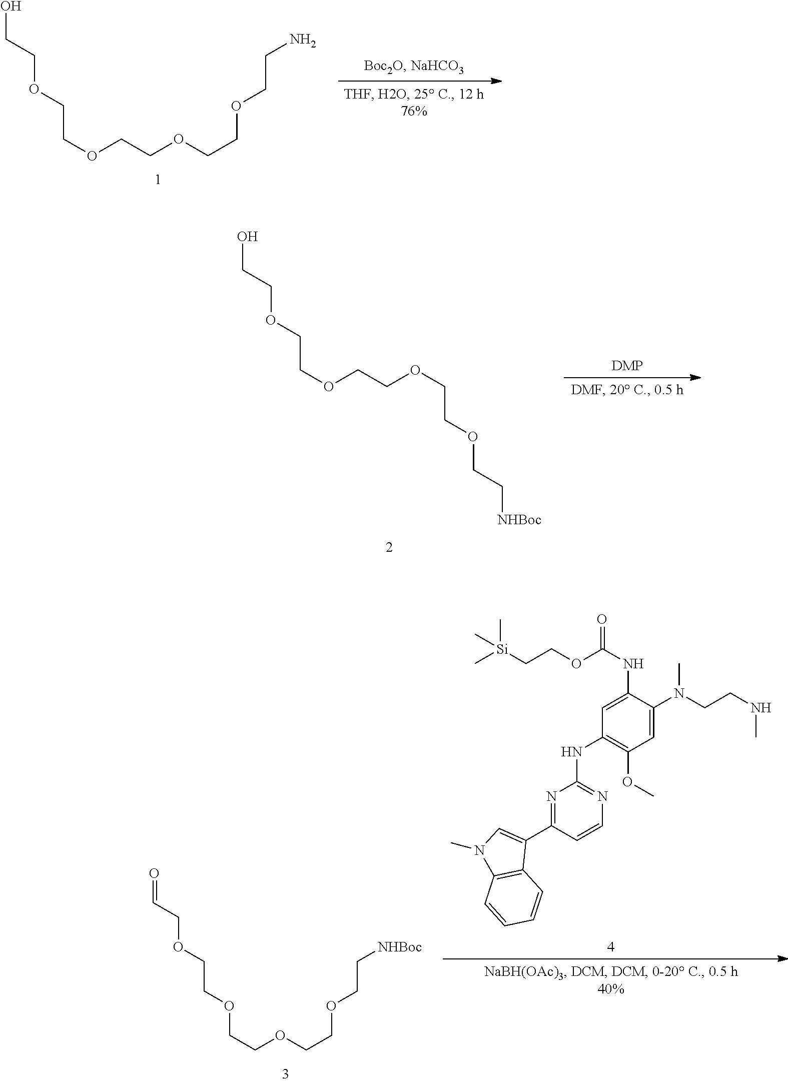

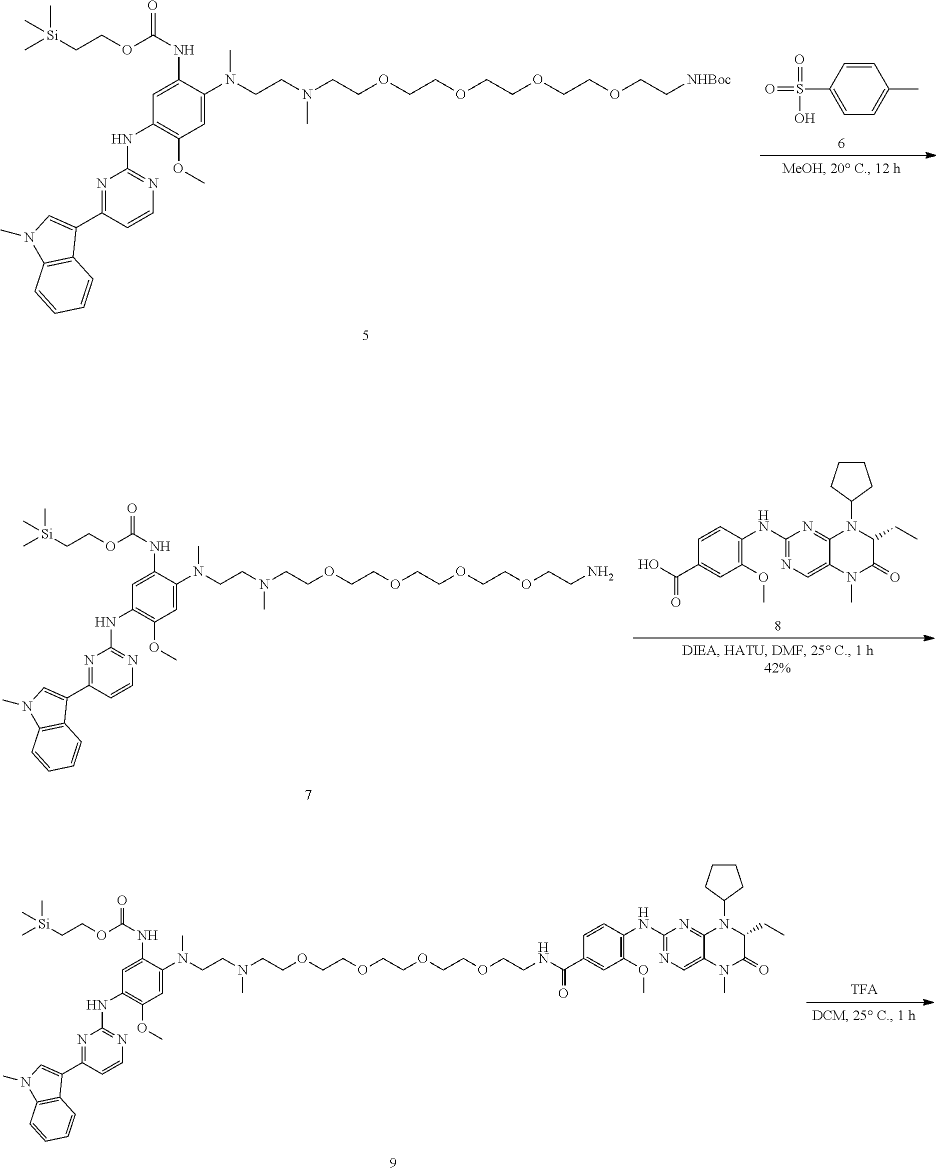

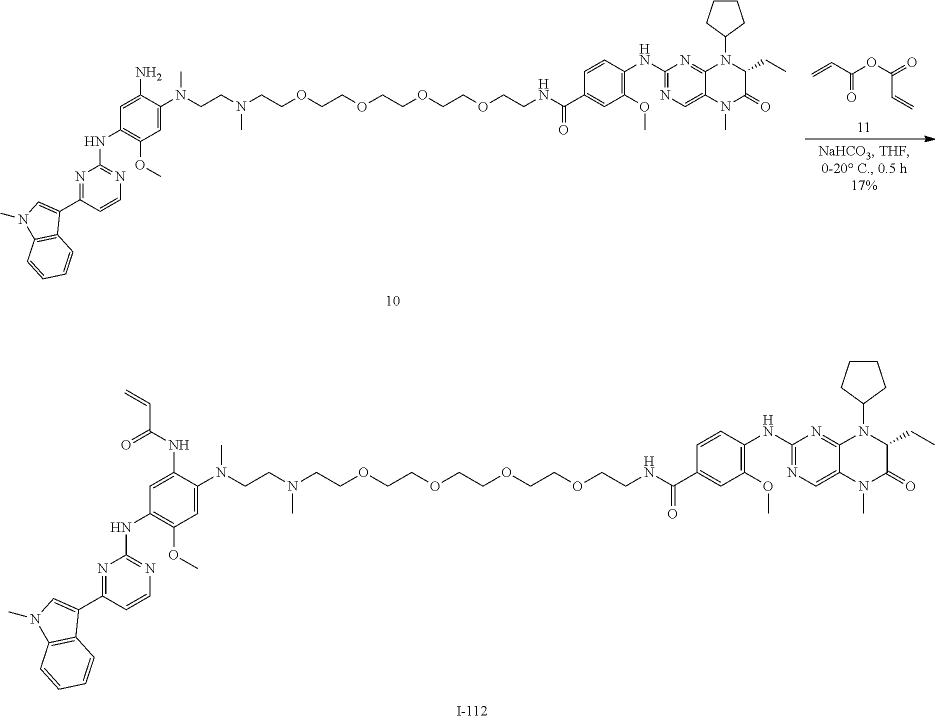

General Information: Compound 8 is a known compound from WO2017/24317, 2017, A2.

Step 1. To a solution of 2-[2-[2-[2-(2-aminoethoxy)ethoxy]ethoxy]ethoxy]ethanol (1.00 g, 4.21 mmol, 1.0 equiv) in THF (10 mL) and H₂O (10 mL) was added Boc₂O (1.84 g, 8.43 mmol, 1.94 mL, 2.0 equiv) and NaHCO₃ (3.54 g, 42.1 mmol, 1.64 mL, 10.0 equiv). The mixture was stirred at 25° C. for 12 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The crude product was purified by column chromatography on silica gel (petroleum ether: ethyl acetate=10:1 to 0:1) to give tert-butyl N-[2-[2-[2-[2-(2-hydroxyethoxy)ethoxy]ethoxy]ethoxy]ethyl]carbamate (1.08 g, 3.20 mmol, 76% yield) as a colorless oil. ¹H NMR (400 MHz, CDCl₃) δ3.90-3.43 (m, 19H), 3.43-2.87 (m, 3H), 1.52-1.38 (m, 9H).

Step 2. To a solution of tert-butyl N-[2-[2-[2-[2-(2-hydroxyethoxy)ethoxy]ethoxy]ethoxy]ethyl]carbamate (235 mg, 696 μmol, 1.0 equiv in DMF (2 mL) was added DMP (1.18 g, 2.79 mmol, 862 μL, 4.0 equiv). The mixture was stirred at 20° C. for 0.5 h. tert-butyl N-[2-[2-[2-[2-(2-oxoethoxy)ethoxy]ethoxy]ethoxy]ethyl]carbamate (233 mg, 694 μmol) was obtained in yellow liquid and it was used by next step directly.

Step 3. To a solution of 2-trimethylsilylethyl N-[4-methoxy-5-[[4-(1-methylindol-3-yl)pyrimidin-2-yl]amino]-2-[methyl-[2-(methylamino)ethyl]amino]phenyl]carbamate (400 mg, 694 μmol, 1.0 equiv) in DCM (10 mL) was added TEA (210 mg, 2.08 mmol, 290 μL, 3.0 equiv), After pH was adjusted to 7-8, NaBH(OAc)₃ (1.47 g, 6.95 mmol, 10.0 equiv) was added. Then tert-butyl N-[2-[2-[2-[2-(2-oxoethoxy)ethoxy]ethoxy]ethoxy]ethyl]carbamate (233 mg, 694 μmol, 1.0 equiv) in DMF (4 mL) was dropwise added at 0° C. The reaction mixture was stirred at 20° C. for 0.5 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by prep-HPLC (column: Waters Xbridge C18 150*50 mm*10 m; mobile phase: [water(NH₄HCO₃)-ACN]; B%: 90%-100%, 11 min) (column: Phenomenex luna C18 150*40 mm*15 m; mobile phase: [water(FA)-ACN]; B%: 27%-57%, 10 min) and (column: Phenomenex luna C18 150*25 mm*10 m; mobile phase: [water(FA)-ACN]; B%: 30%-60%, 7 min) to afford 2-trimethylsilylethyl N—[[2[2-[2-[2-[2-[2-(tert-butoxycarbonylamino)ethoxy]ethoxy]ethoxy]ethoxy]ethyl-methyl-amino]ethyl-methyl-amino]-4-methoxy-5-[[4-(1-methylindol-3-yl)pyrimidin-2-yl]amino]phenyl]carbamate (250 mg, 279 μmol, 40% yield) as a yellow oil.

Step 4. To a solution of 2-trimethylsilylethyl N-[2-[2-[2-[2-[2-[2-[2-(tert-butoxycarbonylamino)ethoxy]ethoxy]ethoxy]ethoxy]ethyl-methyl-amino]ethyl-methyl-amino]-4-methoxy-5-[[4-(1-methylindol-3-yl)pyrimidin-2-yl]amino]phenyl]carbamate (250 mg, 279 μmol, 1.0 equiv) in MeOH (17 mL) was added 4-methylbenzenesulfonic acid (144 mg, 837 μmol, 3.0 equiv). The mixture was stirred at 50° C. for 12 h. The reaction mixture was concentrated in vacuum to afford 2-trimethylsilylethyl N-[2-[2-[2-[2-[2-[2-(2-aminoethoxy)ethoxy]ethoxy]ethoxy]ethyl-methyl-amino]ethyl-methyl-amino]-4-methoxy-5-[[4-(1-methylindol-3-yl)pyrimidin-2-yl]amino]phenyl]carbamate (220 mg, 276 μmol) as a yellow oil and it was used by next step.

Step 5. To a solution of 2-trimethylsilylethyl N-[2-[2-[2-[2-[2-[2-(2-aminoethoxy)ethoxy]ethoxy]ethoxy]ethyl-methyl-amino]ethyl-methyl-amino]-4-methoxy-5-[[4-(1-methylindol-3-yl)pyrimidin-2-yl]amino]phenyl]carbamate (187 mg, 235 μmol, 1.0 equiv) and 4-[(8-cyclopentyl-7-ethyl-5-methyl-6-oxo-7H-pteridin-2-yl)amino]-3-methoxy-benzoic acid (100 mg, 235 μmol, 1.0 equiv) in DMF (3 mL) was added DIEA (151 mg, 1.18 mmol, 204 L, 5.0 equiv) and HATU (134 mg, 352 μmol, 1.5 equiv). The mixture was stirred at 25° C. for 1 h. The residue was purified by prep-HPLC (column: Phenomenex Synergi Polar-RP 100*25 mm*4 μm; mobile phase: [water(TFA)-ACN]; B%: 38%-58%, 7 min) to afford 2-trimethylsilylethyl N—[[2[2-[2-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethyl-methyl-amino]ethyl-methyl-amino]-4-methoxy-5-[[4-(1-methylindol-3-yl)pyrimidin-2-yl]amino]phenyl]carbamate (120 mg, 99.7 μmol, 42% yield) as a white solid.

Step 6. To a solution of 2-trimethylsilylethyl N-[2-[2-[2-[2-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethyl-methyl-amino]ethyl-methyl-amino]-4-methoxy-5-[[4-(1-methylindol-3-yl)pyrimidin-2-yl]amino]phenyl]carbamate (120 mg, 99.7 μmol, 1.0 equiv) in DCM (5 mL) was added TFA (1 mL). The mixture was stirred at 25° C. for 1 h. The reaction mixture was concentrated in vacuum to afford N-[2-[2-[2-[2-[2-[2-[2-amino-5-methoxy-N-methyl-4-[[4-(1-methylindol-3-yl)pyrimidin-2-yl]amino]anilino]ethyl-methyl-amino]ethoxy]ethoxy]ethoxy]ethoxy]ethyl]-4-[[(7R)-8-cyclopentyl-7-ethyl-5-methyl-6-oxo-7H-pteridin-2-yl]amino]-3-methoxy-benzamide (115 mg, 98.1 μmol, crude, TFAsalt) as a yellow oil and it was used by next step directly.

Step 7. To a solution of N-[2-[2-[2-[2-[2-[2-[2-amino-5-methoxy-N-methyl-4-[[4-(1-methylindol-3-yl)pyrimidin-2-yl]amino]anilino]ethyl-methyl-amino]ethoxy]ethoxy]ethoxy]ethoxy]ethyl]-4-[[(7R)-8-cyclopentyl-7-ethyl-5-methyl-6-oxo-7H-pteridin-2-yl]amino]-3-methoxy-benzamide (115 mg, 98.1 μmol, 1.0 equiv, TFA salt) in THF (2 mL) was added NaHCO₃ (82.4 mg, 980 μmol, 38.1 μL, 10.0 equiv) at 0° C., then prop-2-enoyl prop-2-enoate (13.6 mg, 107 μmol, 1.1 equiv) was added at 0° C., and it was stirred at 25° C. for 0.5 h. To the reaction mixture was added water (50 mL) and the mixture was extracted with EtOAc (50 mL). The combined organic phase was washed with brine (50 mL×3), dried over anhydrous Na₂SO₄, filtered and concentrated in vacuum. The residue was purified by prep-HPLC (column: Phenomenex Synergi C18 150*25 mm*10 μm; mobile phase: [water(FA)-ACN]; B%: 13%-43%, 10 min) to afford 4-[[(7R)-8-cyclopentyl-7-ethyl-5-methyl-6-oxo-7H-pteridin-2-yl]amino]-3-methoxy-N-[2-[2-[2-1[2-[2-1[2-1[5-methoxy-N-methyl-4-1[[4-(1-methylindol-3-yl)pyrimidin-2-yl]amino]-2-(prop-2-enoylamino)anilino]ethyl-methyl-amino]ethoxy]ethoxy]ethoxy]ethoxy]ethyl]benzamide (20.28 mg, 17.14 μmol, 17% yield, 94% purity) as a light yellow solid. ¹H NMR (400 MHz, CD₃OD) δ8.96-8.76 (m, 1H), 8.57-8.48 (m, 1H), 8.42 (d, J=8.3 Hz, 1H), 8.36-8.20 (m, 3H), 7.72 (s, 1H), 7.53-7.36 (m, 3H), 7.28-7.12 (m, 3H), 6.93 (s, 1H), 6.64-6.37 (m, 2H), 5.84 (m, 1H), 4.41 (m, 1H), 4.23 (m, 1H), 3.96 (d, J=13.6 Hz, 6H), 3.91-3.85 (m, 3H), 3.67-3.36 (m, 21H), 3.28 (s, 3H), 3.23-3.14 (m, 3H), 2.83-2.67 (m, 5H), 2.17-1.63 (m, 1OH). LC-MS: MS (ES⁺): RT=1.631 min, m/z=1112.5 [M+H⁺]; LCMS Method: 25.

### Example 97—Synthesis of Compound I-113

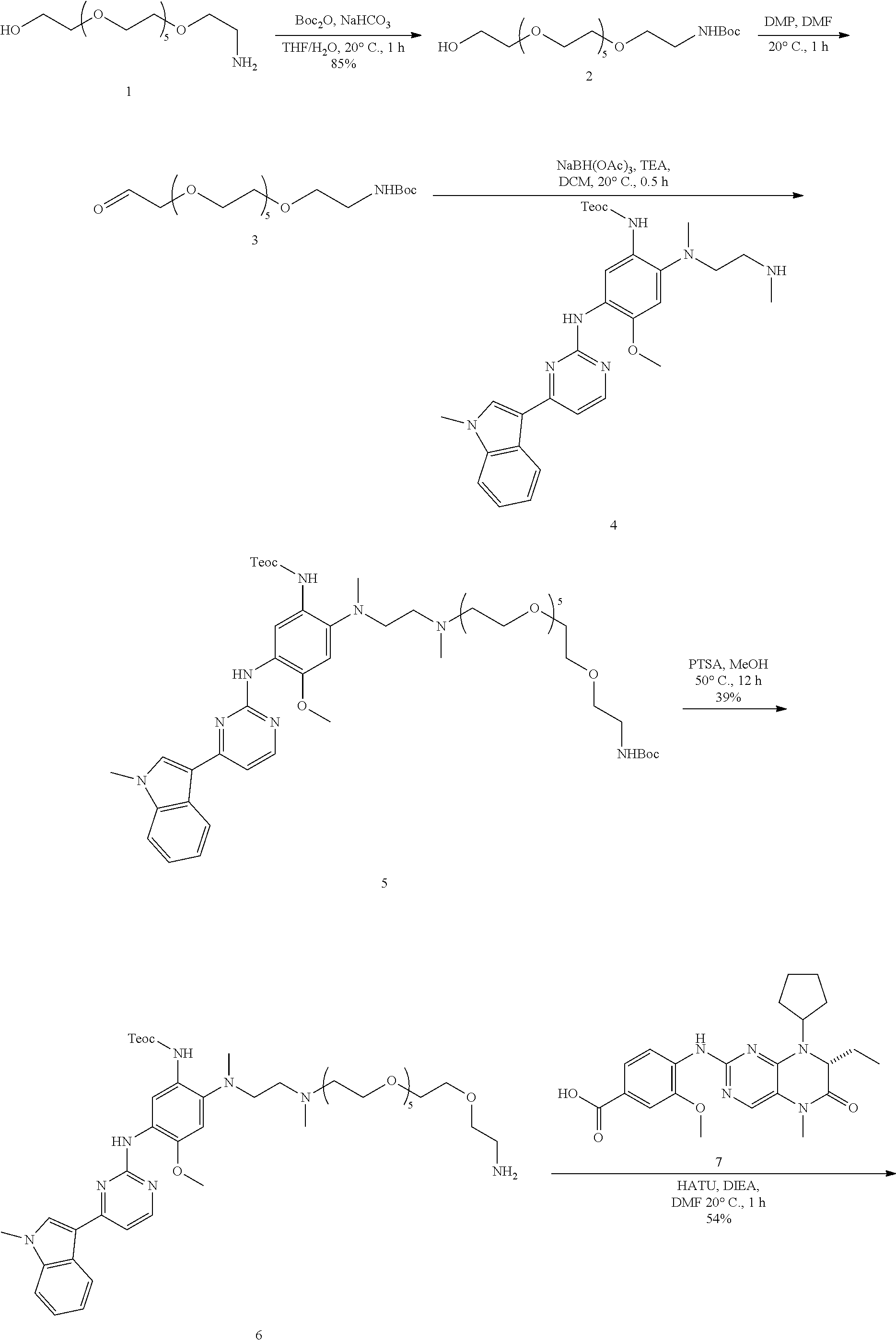

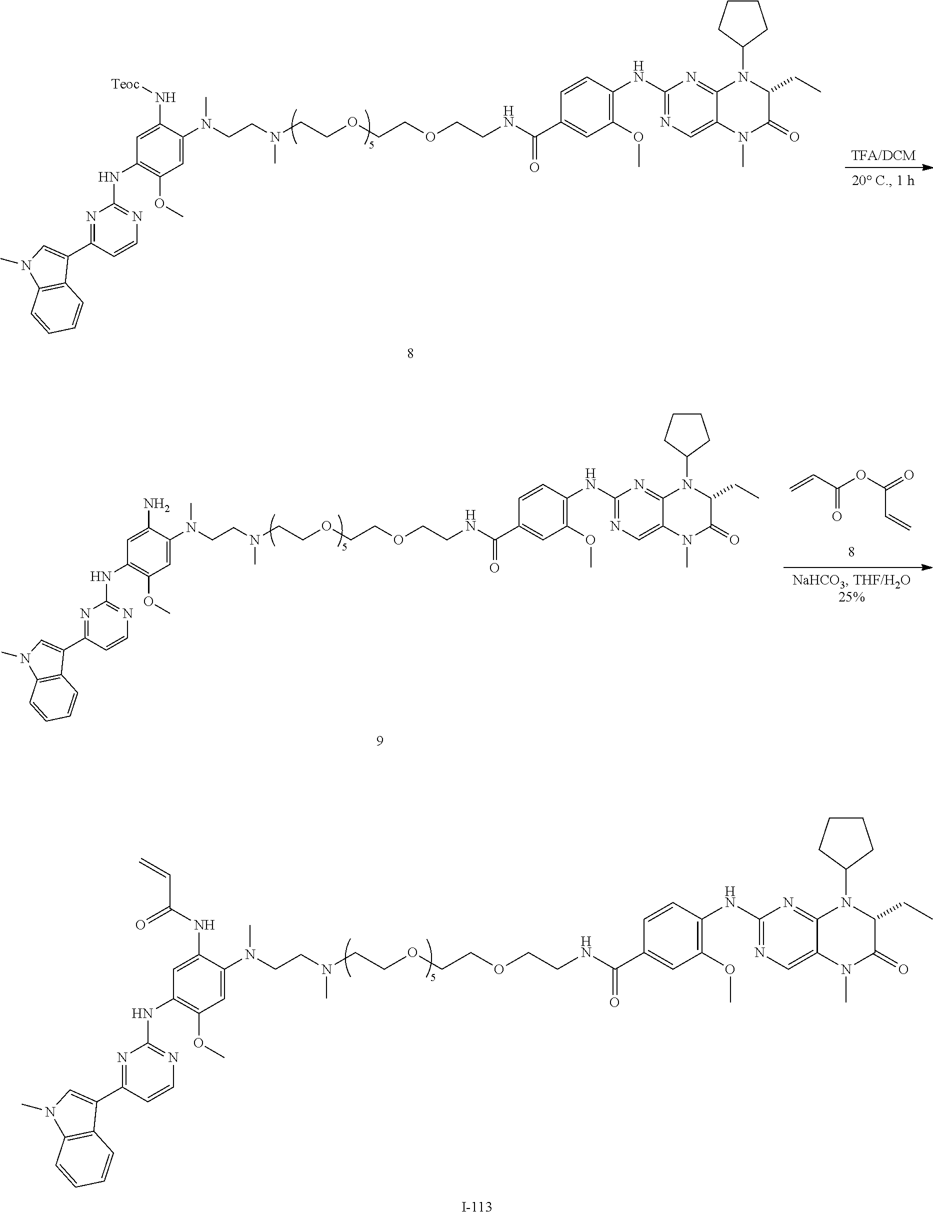

General Information: Compound 7 is a known compound from WO2017/24317, 2017, A2.

Step 1. To a solution of compound 1 (500 mg, 1.54 mmol, 1.0 equiv) and in THF (1 mL) and NaHCO₃ (1.29 g, 15.37 mmol, 597 μL, 10 equiv) in H₂O (1 mL) was added (Boc)₂O (670 mg, 3.07 mmol, 706 μL, 2.0 equiv). The mixture was stirred at 20° C. for 1 h. The reaction mixture was partitioned between H₂O (30 mL) and EtOAc (3×50 mL). The organic phase was separated, washed with brine (5 mL), dried over Na₂SO₄, filtered and concentrated under reduced pressure to give a residue. The residue was purified by column chromatography (SiO₂, Petroleum ether/Ethyl acetate=1/1 to 0/1). Compound 2 (560 mg, 1.32 mmol, 85% yield) was obtained as a yellow oil. ¹HNMR (400 MHz, CDCl₃): δ 9.78 (t, J=1.6 Hz, 1H), 4.03 (s, 2H), 3.71 (d, J=3.6 Hz, 2H), 3.68-3.63 (m, 17H), 3.61-3.55 (m, 2H), 3.48 (t, J=6.2 Hz, 2H), 2.50-2.46 (m, 1H), 1.71 (m 2H), 1.48 (s, 9H)

Step 2. To a solution of compound 2 (300 mg, 705 μmol, 1.0 equiv) in DCM (10 mL) was added DMP (897 mg, 2.12 mmol, 654 μL, 3.0 equiv), and then it was stirred at 20° C. for 1 h. Compound 3 (300 mg, crude) was used into the next step without further purification.

Step 3. To a solution of compound 4 (407 mg, 708 μmol, 1.0 equiv) in DCM (10 mL) was added NaBH(OAc)₃ (750 mg, 3.54 mmol, 5.0 equiv), Et₃N (752 mg, 7.43 mmol, 1.0 mL, 10 equiv) and compound 3 (300 mg, 708 μmol, 1.0 equiv). The mixture was stirred at 20° C. for 0.5 h. The reaction mixture was partitioned between DCM (10 mL) and NaHCO₃ (10 mL). The organic phase was separated, washed with brine (10 mL), dried over Na₂SO₄, filtered and concentrated under reduced pressure to give a residue. The residue was purified by prep-HPLC (column: Phenomenex luna C18 150*40 mm*15 m; mobile phase: [water (TFA)-ACN]; B%: 33%-63%, 11 min). Compound 5 (300 mg, crude) was obtained as a yellow solid.

Step 4. To a solution of compound 5 (250 mg, 254 μmol, 1.0 equiv) in MeOH (5 mL) was added PTSA (87 mg, 508 μmol, 2.0 equiv). The mixture was stirred at 50° C. for 12 h. The reaction mixture was concentrated under reduced pressure to give a residue. The residue was purified by prep-HPLC (column: Phenomenex luna C18 150*40 mm*15 m; mobile phase: [water (TFA)-ACN]; B%: 20%-50%, 11 min). Compound 6 (100 mg, 100 μmol, 39% yield, TFA salt) was obtained as a yellow solid.

Step 5. To a solution of compound 6 (90 mg, 90 μmol, 1.0 equiv, TFA salt) and compound 7 (42 mg, 99 μmol, 1.1 equiv) in DMF (1 mL) was added DIEA (34 mg, 270 μmol, 47 μL, 3.0 equiv) and HATU (51 mg, 135 μmol, 1.5 equiv). The mixture was stirred at 20° C. for 1 h. The reaction mixture filtered and concentrated under reduced pressure to give a residue. The residue was purified by prep-HPLC (column: 3_Phenomenex Luna C18 75*30 mm*3 m; mobile phase: [water (TFA)-ACN]; B%: 33%-63%, 7 min) to afford compound 8 (70 mg, 48 μmol, 54% yield, 90% purity) as a yellow solid.

Step 6. To a solution of compound 8 (56 mg, 30 μmol, 70% purity, 1.0 equiv) in DCM (1 mL) was added TFA (0.5 mL). The mixture was stirred at 20° C. for 1 h. The reaction mixture was concentrated under reduced pressure to give compound 9 (50 mg, 27 μmol, 70% purity, TFA salt) as a yellow oil.

Step 7. To a solution of compound 9 (50 mg, 27 μmol, 70% purity, 1.0 equiv, TFA salt) in THF (1 mL) was added NaHCO₃ (23 mg, 277 μmol, 10 equiv) in H₂O (1 mL) and compound 8 (3.5 mg, 27 μmol, 1.0 equiv). The mixture was stirred at 20° C. for 0.5 h. The reaction mixture was partitioned between NaHCO₃ (10 mL) and EtOAc (10 mL). The organic phase was separated, washed with brine (10 mL), dried over Na₂SO₄, filtered and concentrated under reduced pressure to give a residue. The residue was purified by prep-HPLC (column: Phenomenex Synergi C18 150*25 mm*10 μm; mobile phase: [water(FA)-ACN]; B%: 13%-40%, 9 min) and further purified by prep-HPLC (column: Waters Xbridge 150*25 mm*5 m; mobile phase: [water(NH₄HCO₃)-ACN]; B%: 60%-90%, 8 min) to afford (R)—N-(2-(2-acrylamido-5-methoxy-4-((4-(1-methyl-1H-indol-3-yl)pyrimidin-2-yl)amino)phenyl)-5-methyl-8,11,14,17,20,23-hexaoxa-2,5-diazapentacosan-25-yl)-4-((8-cyclopentyl-7-ethyl-5-methyl-6-oxo-5,6,7,8-tetrahydropteridin-2-yl)amino)-3-methoxybenzamide (8 mg, 7 μmol, 25% yield, 99% purity) as a yellow gum. ¹HNMR (400 MHz, CD₃OD): 69.56-9.44 m, 1H), 8.68 (s, 1H), 8.44 (d, J=8.4 Hz, 1H), 8.27 (d, J=5.5 Hz, 1H), 8.13 (d, J=7.8 Hz, 1H), 7.72 (s, 1H), 7.51-7.40 (m, 3H), 7.28-7.15 (m, 3H), 6.97 (s, 1H), 6.66 (m, 1H), 6.40 (m 1H), 5.82-5.74 (m, 1H), 4.46-4.36 (m, 1H), 4.26-4.19 (m, 1H), 4.00-3.88 (m, 9H), 3.66-3.57 (m, 6H), 3.56-3.51 (m, 6H), 3.50-3.47 (m, 2H), 3.45-3.37 (m, 12H), 3.28 (s, 4H), 3.01 (s, 2H), 2.70 (s, 3H), 2.67-2.59 (m, 2H), 2.56-2.46 (m, 2H), 2.33 (s, 3H), 2.18-2.08 (m, 1H), 1.96-1.64 (m, 9H), 0.81 (t, J=7.5 Hz, 3H). LC-MS: MS (ES′): RT=1.687 min, m/z=600.9 [M/2+H⁺]; LCMS method: 25.

### Example 98—Synthesis of Compound I-114

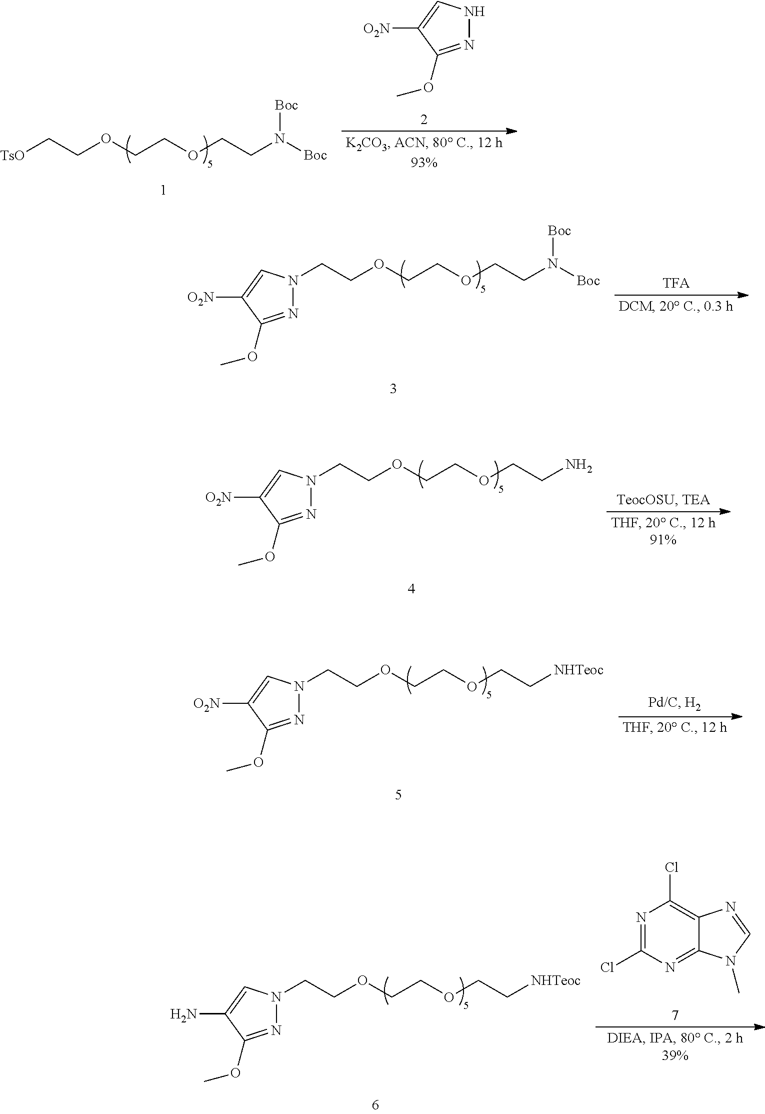

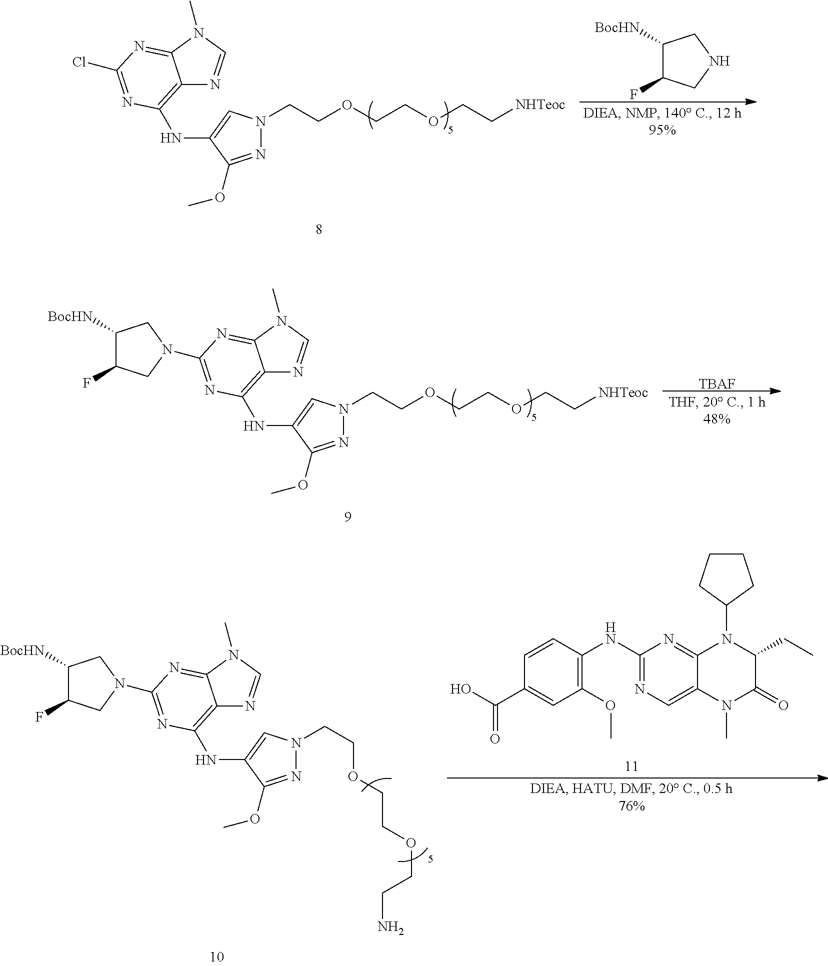

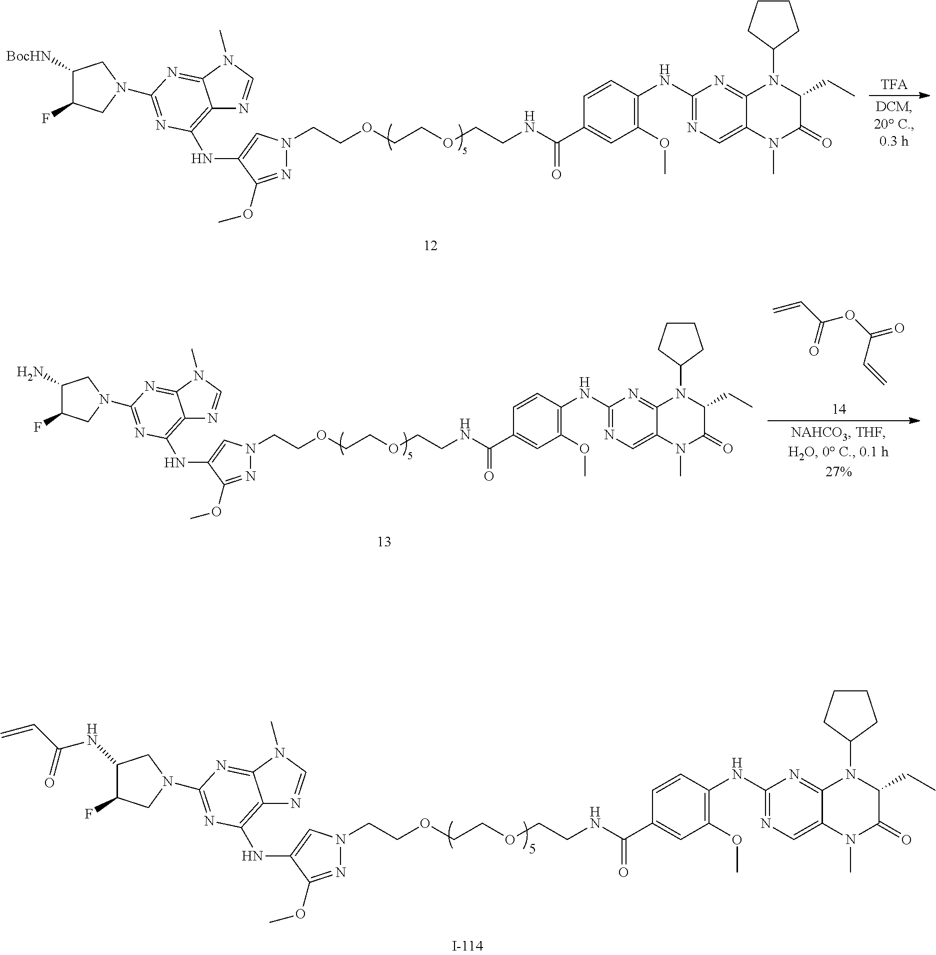

General Information: Compound 11 is a known compound from WO2018/148443, 2018, A1.

Step 1. To a solution of 2-[[2[2-[2-[2-[2-[bis(tert-butoxycarbonyl)amino]ethoxy]ethoxy]ethoxy]ethoxy]ethoxy]ethoxy]ethyl 4-methylbenzenesulfonate (2.7 g, 3.97 mmol, 1.0 equiv) in ACN (30 mL) was added K₂CO₃ (1.7 g, 11.9 mmol, 3.0 equiv) and 3-methoxy-4-nitro-1H-pyrazole (568 mg, 3.97 mmol, 1.0 equiv). The mixture was stirred at 80° C. for 12 h. The residue was purified by column chromatography (SiO₂, Petroleum ether/Ethyl acetate=1/1 to 0/1) to give tert-butyl N-tert-butoxycarbonyl-N-[2-[2-[2-[2-[2-[2-[2-(3-methoxy-4-nitro-pyrazol-1-yl)ethoxy]ethoxy]ethoxy]ethoxy]ethoxy]ethoxy]ethyl]carbamate (2.4 g, 93% yield).

Step 2. To a solution of tert-butyl N-tert-butoxycarbonyl-N-[2-[2-[2-[2-[2-[2-[2-(3-methoxy-4-nitro-pyrazol-1-yl)ethoxy]ethoxy]ethoxy]ethoxy]ethoxy]ethoxy]ethyl]carbamate (2.4 g, 3.69 mmol, 1.0 equiv) in DCM (20 mL) was added TFA (4 mL). The mixture was stirred at 20° C. for 0.3 h and concentrated to give 2-[2-[2-[2-[2-[2-[2-(3-methoxy-4-nitro-pyrazol-1-yl)ethoxy]ethoxy]ethoxy]ethoxy]ethoxy]ethoxy]ethanamine (2.0 g, TFA salt) as a yellow oil.

Step 3. To a solution of 2-[2-[2-[2-[2-[2-[2-(3-methoxy-4-nitro-pyrazol-1-yl)ethoxy]ethoxy]ethoxy]ethoxy]ethoxy]ethoxy]ethanamine (2.0 g, 3.68 mmol, 1.0 equiv, TFA salt) in THF (15 mL) was added and TEA (1.86 g, 18.4 mmol, 5.0 equiv) and (2,5-dioxopyrrolidin-1-yl) 2-trimethylsilylethyl carbonate (1.43 g, 5.53 mmol, 1.5 equiv). The mixture was stirred at 20° C. for 12 h. The residue was purified by column chromatography (SiO₂, Petroleum ether/Ethyl acetate=1/1 to 0/1) to give 2-trimethylsilylethyl N-[2-[2-[2-[2-[2-[2-[2-(3-methoxy-4-nitro-pyrazol-1-yl)ethoxy]ethoxy]ethoxy]ethoxy]ethoxy]ethoxy]ethyl]carbamate (2.0 g, 91% yield). ¹H NMR (400 MHz, CDCl₃): δ8.24 (s, 1H), 5.29 (s, 1H), 4.19-4.15 (m, 4H), 4.06 (s, 3H), 3.88-3.84(m, 2H), 3.69-3.63 (m, 20H), 3.57 (t, J=5.2 Hz, 2H), 3.40-3.38 (m, 2H), 1.04-0.95 (m, 2H), 0.06 (s, 9H)

Step 4. To a solution of 2-trimethylsilylethyl N-[2-[2-[2-[2-[2-[2-[2-(3-methoxy-4-nitro-pyrazol-1-yl)ethoxy]ethoxy]ethoxy]ethoxy]ethoxy]ethoxy]ethyl]carbamate (2.0 g, 3.36 mmol, 1.0 equiv) in THF (60 mL) was added Pd/C (0.5 g, 10% purity). The mixture was stirred under H₂ (15 psi) at 20° C. for 12 h. The mixture was filtered and concentrated to give 2-trimethylsilylethylN-[2-[2-[2-[2-[2-[2-[2-(4-amino-3-methoxy-pyrazol-1-yl)ethoxy]ethoxy]ethoxy]ethoxy]ethoxy]ethoxy]ethyl]carbamate (1.9 g, crude).

Step 5. To a solution of 2,6-dichloro-9-methyl-purine (575 mg, 2.83 mmol, 1.0 equiv) in IPA (10 mL) was added DIEA (1.1 g, 8.5 mmol, 1.5 mL, 3.0 equiv) and 2-trimethylsilylethyl N-[22[2-1[2-[2-[2-1[2-(4-amino-3-methoxy-pyrazol-1-yl)ethoxy]ethoxy]ethoxy]ethoxy]ethoxy]ethoxy]ethyl]carbamate (1.6 g, 2.83 mmol, 1.0 equiv). The mixture was stirred at 80° C. for 2 h and purified by prep-HPLC (column: Waters Xbridge C18 150*50 mm*10 μm; mobile phase: [water(NH₄HCO₃)-ACN]; B%:36%-66%, 11 min) to give 2-trimethylsilylethyl N-[2-[2-[12[2-1[2-1[2-[4-[(2-chloro-9-methyl-purin-6-yl)amino]-3-methoxy-pyrazol-1-yl]ethoxy]ethoxy]ethoxy]ethoxy]ethoxy]ethoxy]ethyl]carbamate (0.8 g, 39% yield).

Step 6. To a solution of tert-butyl N-[(3R,4R)-4-fluoropyrrolidin-3-yl]carbamate (167 mg, 820 μmol, 1.0 equiv) in NMP (4 mL) was added DIEA (318 mg, 2.46 mmol, 3.0 equiv) and 2-trimethylsilylethyl N-[2[2-[2-[2-2-[2-[2-[4-[(2-chloro-9-methylpurin-6-yl)amino]-3-methoxy-pyrazol-1-yl]ethoxy]ethoxy]ethoxy]ethoxy]ethoxy]ethoxy]ethyl]carbamate (600 mg, 820 μmol, 1.0 equiv). The mixture was stirred at 140° C. for 12 h and purified by prep-HPLC (column: Waters Xbridge C18 150*50 mm*10 μm; mobile phase: [water(NH4HCO₃)-ACN]; B%: 44%-74%, 11 min) to give the desired product 2-trimethylsilylethyl N-[2-[2-[2-[2-[2-[2-[2-[4-[[2-[(3R,4R)-3-(tert-butoxycarbonylamino)-4-fluoro-pyrrolidin-1-yl]-9-methyl-purin-6-yl]amino]-3-methoxy-pyrazol-1-yl]ethoxy]ethoxy]ethoxy]ethoxy]ethoxy]ethoxy]ethyl]carbamate (700 mg, 95% yield) as a yellow oil.

Step 7. To a solution of 2-trimethylsilylethyl N-[2-[2-[2-[2-[2-[2-[2-[4-[[2-[(4R)-3-(tert-butoxycarbonylamino)-4-fluoro-pyrrolidin-1-yl]-9-methyl-purin-6-yl]amino]-3-methoxy-pyrazol-1-yl]ethoxy]ethoxy]ethoxy]ethoxy]ethoxy]ethoxy]ethyl]carbamate (300 mg, 333 μmol, 1.0 equiv) in THF (5 mL) was added TBAF (1 M, 1.7 mL, 5.0 equiv). The mixture was stirred at 20° C. for 1 h. The residue was purified by prep-TLC (DCM/MeOH=8/1) to give tert-butyl N-[(4R)-1-[6-[[1-[2-[2-1[2-[2-1[2-[2-(2-aminoethoxy)ethoxy]ethoxy]ethoxy]ethoxy]ethoxy]ethyl]-3-methoxy-pyrazol-4-yl]amino]-9-methyl-purin-2-yl]-4-fluoro-pyrrolidin-3-yl]carbamate (120 mg, 48% yield).

Step 8. To a solution of 4-[[(7R)-8-cyclopentyl-7-ethyl-5-methyl-6-oxo-7H-pteridin-2-yl]amino]-3-methoxy-benzoic acid (67 mg, 158 μmol, 1.0 equiv), tert-butyl N-[(4R)-1-[6-[[1-[12[2-[2-[2-[2-(2-aminoethoxy)ethoxy]ethoxy]ethoxy]ethoxy]ethoxy]ethyl]-3-methoxy-pyrazol-4-yl]amino]-9-methyl-purin-2-yl]-4-fluoro-pyrrolidin-3-yl]carbamate (120 mg, 158 μmol, 1.0 equiv) in DMF (3 mL) was added HATU (72 mg, 190 μmol, 1.2 equiv) and DIEA (61 mg, 476 μmol, 83 μL, 3.0 equiv). The mixture was stirred at 20° C. for 0.5 h. The residue was purified by prep-HPLC (column: Phenomenex luna C18 250*50 mm*15 m; mobile phase: [water(FA)-ACN]; B%: 31%-61%, 10 min) to give tert-butylN-[(3R,4R)-1-[6-[[1-[2-[2-[2-[2-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethoxy]ethoxy]ethyl]-3-methoxy-pyrazol-4-yl]amino]-9-methyl-purin-2-yl]-4-fluoro-pyrrolidin-3-yl]carbamate (140 mg, 76% yield).

Step 9. To a solution of tert-butyl N-[(4R)-1-[6-[[1-[2-[2-[2-[2-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethoxy]ethoxy]ethyl]-3-methoxy-pyrazol-4-yl]amino]-9-methyl-purin-2-yl]-4-fluoro-pyrrolidin-3-yl]carbamate (70 mg, 60 μmol, 1.0 equiv) in DCM (3 mL) was added TFA (0.5 mL). The mixture was stirred at 20° C. for 0.3 h and concentrated to give N-[2-[121[2-1[2-1[2-[2-[4-[[2-[(4R)-3-amino-4-fluoro-pyrrolidin-1-yl]-9-methyl-purin-6-yl]amino]-3-methoxy-pyrazol-1-yl]ethoxy]ethoxy]ethoxy]ethoxy]ethoxy]ethoxy]ethyl]-4-[[(7R)-8-cyclopentyl-7-ethyl-5-methyl-6-oxo-7H-pteridin-2-yl]amino]-3-methoxy-benzamide (70 mg, TFA salt).

Step 10. To a solution of N-[2-[2-[2-[2-[2-[2-[2-[4-[[2-[(4R)-3-amino-4-fluoro-pyrrolidin-1-yl]-9-methyl-purin-6-yl]amino]-3-methoxypyrazol-1-yl]ethoxy]ethoxy]ethoxy]ethoxy]ethoxy]ethoxy]ethyl]-4-[ [(7R)-8-cyclopentyl-7-ethyl-5-methyl-6-oxo-7H-pteridin-2-yl]amino]-3-methoxy-benzamide (70 mg, 59 μmol, 1.0 equiv, TFA salt) in THF (1.5 mL) was added NaHCO₃ (0.5 mL) and prop-2-enoyl prop-2-enoate (8 mg, 60 μmol, 1.0 equiv) at 0° C. The mixture was stirred at 0° C. for 0.1 h and purified by prep-HPLC (column: Phenomenex luna C18 250*50 mm*15 m; mobile phase: [water(FA)-ACN]; B%: 24%-54%, 10 min) to give 4-[[(7R)-8-cyclopentyl-7-ethyl-5-methyl-6-oxo-7H-pteridin-2-yl]amino]-N-[2-[2-[2-[2-[2-[2-[2-[4-[[2-[(3R)-3-fluoro-4-(prop-2-enoylamino)pyrrolidin-1-yl]-9-methyl-purin-6-yl]amino]-3-methoxy-pyrazol-1-yl]ethoxy]ethoxy]ethoxy]ethoxy]ethoxy]ethoxy]ethyl]-3-methoxy-benzamide (20 mg, 27% yield). ¹H NMR (400 MHz, CD₃OD): δ8.43-8.36 (m, 1H), 8.33 (d, J=8.6 Hz, 1H), 8.14 (s, 1H), 7.73 (d, J=7.2 Hz, 2H), 7.52-7.44 (m, 2H), 6.30 (d, J=6.0 Hz, 2H), 5.69 (t, J=6.0 Hz, 1H), 5.29-5.11 (m, 1H), 4.66-4.62 (m, 1H), 4.48-4.42 (m, 1H), 4.32 (dd, J=7.2, 3.6 Hz, 1H), 4.13 (t, J=4.8 Hz, 2H), 3.99 (d, J=3.6 Hz, 8H), 3.87-3.91 (m, 2H), 3.79 (t, J=4.8 Hz, 2H), 3.71-3.61 (m, 1OH), 3.60-3.55 (m, 8H), 3.53-3.46 (m, 6H), 3.44-3.40 (m, 4H), 2.20-1.28 (m, 12H), 0.85 (t, J=7.6 Hz, 3H). LC-MS: MS (ES′): RT=2.209 min, m/z=1116.5 [M+H⁺]; LCMS method: 10.

### Example 99—Synthesis of Compound I-115

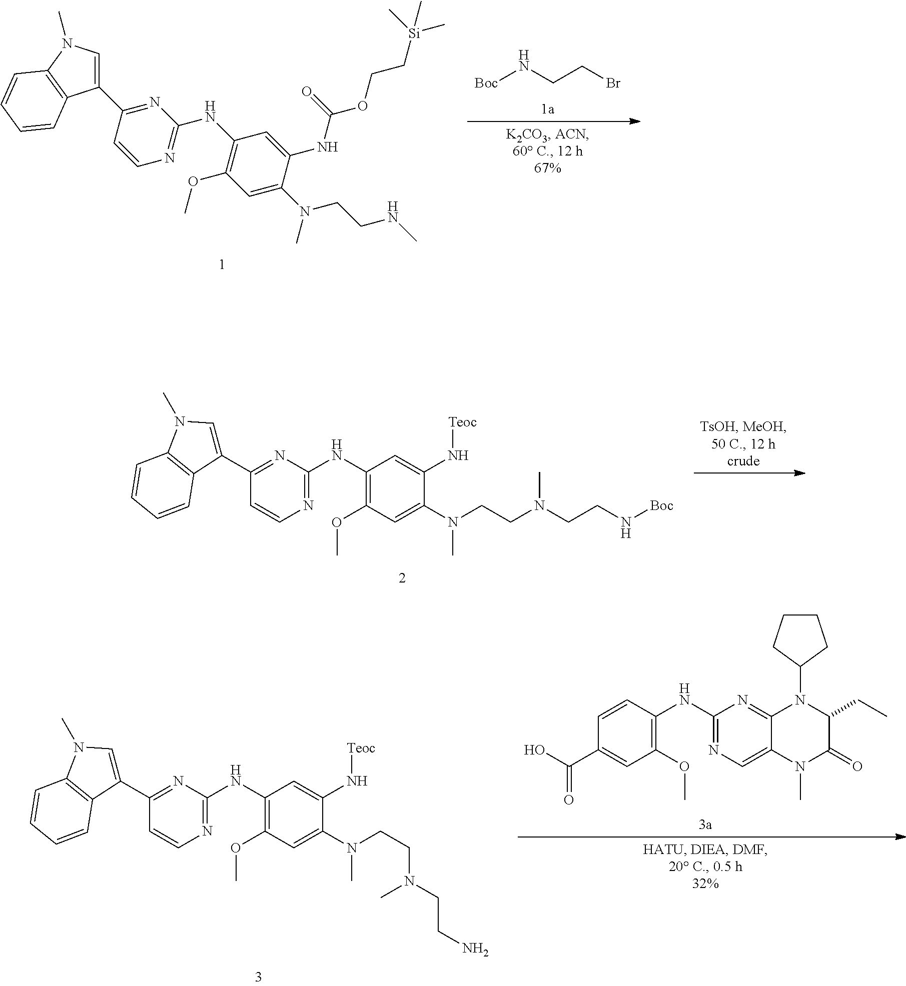

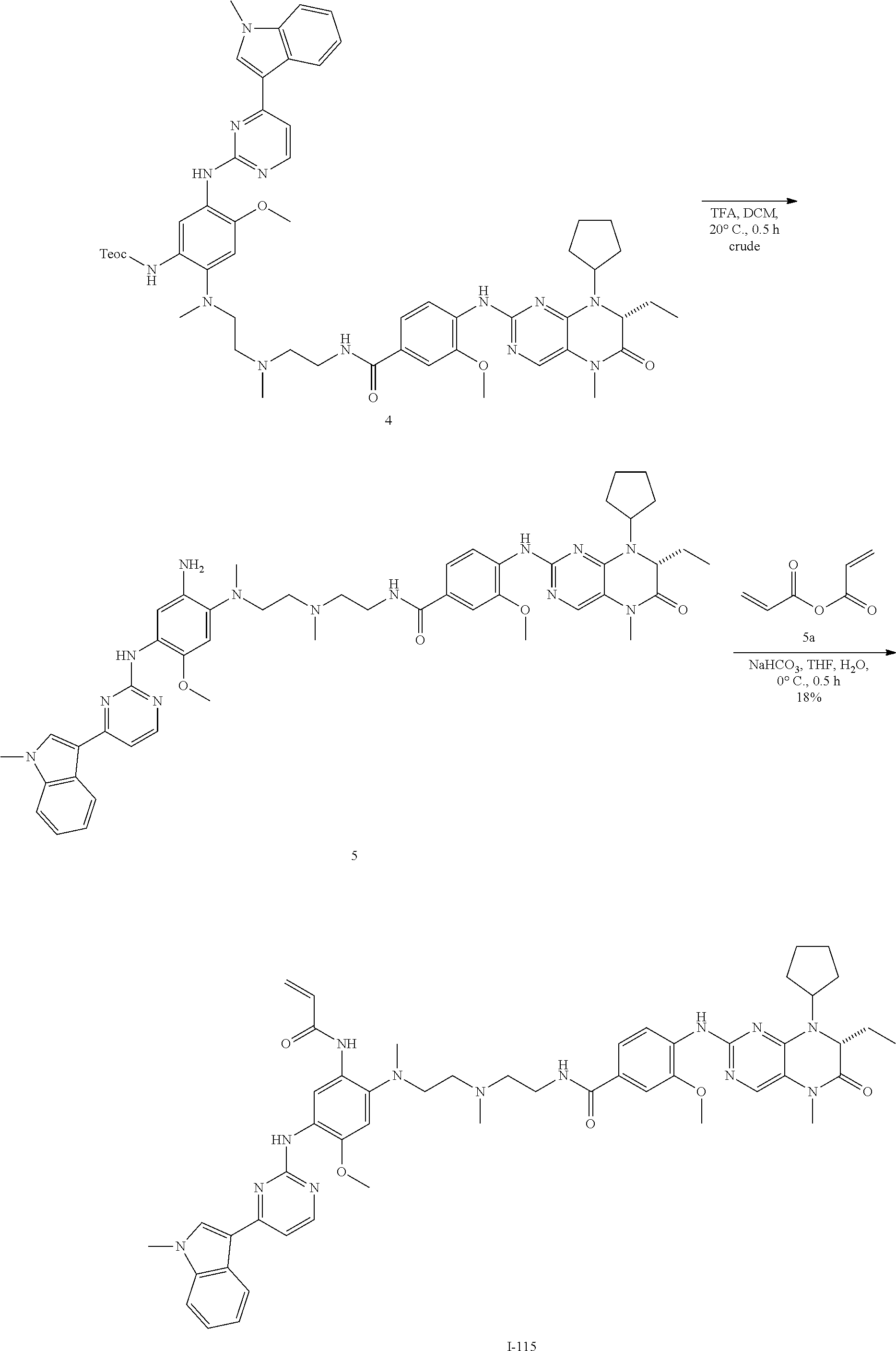

Step 1. To a solution of 2-trimethylsilylethyl N-[4-methoxy-5-[[4-(1-methylindol-3-yl)pyrimidin-2-yl]amino]-2-[methyl-[2-(methylamino)ethyl]amino]phenyl]carbamate (300 mg, 521 μmol, 1 equiv) and tert-butyl N-(2-bromoethyl)carbamate (467 mg, 2.08 mmol, 4 equiv) in ACN (20 mL) was added K₂CO₃ (216 mg, 1.56 mmol, 3 equiv). The mixture was stirred at 60° C. for 12 h and concentrated. The residue was purified by column chromatography (SiO₂, Petroleum ether/Ethyl acetate=20/1 to 1/1) to give 2-trimethylsilylethyl N-[2-[2-[2-(tert-butoxycarbonylamino)ethyl-methyl-amino]ethyl-methyl-amino]-4-methoxy-5-[[4-(1-methylindol-3-yl)pyrimidin-2-yl]amino]phenyl]carbamate (250 mg, 67% yield). ¹H NMR (400 MHz, DMSO) δ 8.80-8.94 (m, 1H), 8.43-8.63 (m, 1H), 8.16-8.36 (m, 2H), 7.74-7.96 (m, 1H), 7.17-7.26 (m, 2H), 7.08-7.15 (m, 1H), 6.91-7.01 (m, 1H), 4.04-4.15 (m, 2H), 3.77-3.92 (m, 7H), 3.51-3.57 (m, 2H), 3.03-3.17 (m, 2H), 2.75-2.87 (m, 2H), 2.61-2.69 (m, 3H), 2.40 (s, 2H), 2.18-2.28 (m, 3H), 1.25-1.39 (m, 10H), 0.93-1.04 (m, 2H), 0.00 (s, 9H).

Step 2. To a solution of 2-trimethylsilylethyl N-[2-[2-[2-(tert-butoxycarbonylamino)ethyl-methyl-amino]ethyl-methyl-amino]-4-methoxy-5-[[4-(1-methylindol-3-yl)pyrimidin-2-yl]amino]phenyl]carbamate (250 mg, 348 μmol, 1 equiv) in MeOH (4 mL) was added TosOH (120 mg, 695 μmol, 2 equiv). The mixture was stirred at 50° C. The mixture was filtered and concentrated to give crude product 2-trimethylsilylethyl N-[2-[2-[2-aminoethyl(methyl)amino]ethyl-methyl-amino]-4-methoxy-5-[[4-(1-methylindol-3-yl)pyrimidin-2-yl]amino]phenyl]carbamate (210 mg) was a yellow oil.

Step 3. To a solution of 2-trimethylsilylethyl N—[2-[2-[2-aminoethyl(methyl)amino]ethyl-methyl-amino]-4-methoxy-5-[ [4-(1-methylindol-3-yl)pyrimidin-2-yl]amino]phenyl]carbamate (190 mg, 307 μmol, 1 equiv) in DMF (2.5 mL) and 4-[[(7R)-8-cyclopentyl-7-ethyl-5-methyl-6-oxo-7H-pteridin-2-yl]amino]-3-methoxy-benzoic acid (131 mg, 307 μmol, 1 equiv) was added DIEA (198 mg, 1.54 mmol, 5 equiv) and HATU (140 mg, 368 μmol, 1.2 equiv). The mixture was stirred at 20° C. for 0.5 h and concentrated. The residue was purified by prep-HPLC (column: Waters Xbridge C18 150*50 mm*10 m; mobile phase: [water(NH4HCO₃)-ACN]; B %: 70%-100%, 11 min) to give 2-trimethylsilylethyl N-[2-[2-[2-[[4-[[(7R)-8-cyclopentyl-7-ethyl-5-methyl-6-oxo-7H-pteridin-2-yl]amino]-3-methoxy-benzoyl]amino]ethyl-methyl-amino]ethyl-methyl-amino]-4-methoxy-5-[ [4-(1-methylindol-3-yl)pyrimidin-2-yl]amino]phenyl]carbamate (100 mg, 32% yield).

Step 5. To a solution of 2-trimethylsilylethyl N-[2-[2-[2-[[4-[[(7R)-8-cyclopentyl-7-ethyl-5-methyl-6-oxo-7H-pteridin-2-yl]amino]-3-methoxy-benzoyl]amino]ethyl-methyl-amino]ethyl-methyl-amino]-4-methoxy-5-[ [4-(1-methylindol-3-yl)pyrimidin-2-yl]amino]phenyl]carbamate (50 mg, 49 μmol, 1 equiv) in CH₂Cl₂ (2 mL) was added TFA (1 mL). The mixture was stirred at 20° C. for 0.5 h. The mixture was filtered and concentrated to give crude product N-[2-[2-[2-amino-5-methoxy-N-methyl-4-[[4-(1-methylindol-3-yl)pyrimidin-2-yl]amino]anilino]ethyl-methyl-amino]ethyl]-4-[[(7R)-8-cyclopentyl-7-ethyl-5-methyl-6-oxo-7H-pteridin-2-yl]amino]-3-methoxy-benzamide (48 mg, TFA salt).

Step 6. To a solution of N-[2-[2-[2-amino-5-methoxy-N-methyl-4-[[4-(1-methylindol-3-yl)pyrimidin-2-yl]amino]anilino]ethyl-methyl-amino]ethyl]-4-[[(7R)-8-cyclopentyl-7-ethyl-5-methyl-6-oxo-7H-pteridin-2-yl]amino]-3-methoxy-benzamide (48 mg, 48 μmol, 1 equiv, TFA salt) in THF (1 mL) was added NaHCO₃ (4 mg, 48 μmol, 1 equiv) and H₂O (0.5 mL). Then prop-2-enoyl prop-2-enoate (6 mg, 48 μmol, 1 equiv) was added at 0° C. The mixture was stirred at 0° C. for 0.5 h. The residue was purified by prep-HPLC (column: Phenomenex luna C18 150*25 mm*10 m; mobile phase: [water(FA)-ACN]; B %: 12%-42%, 10 min) to give 4-[[(7R)-8-cyclopentyl-7-ethyl-5-methyl-6-oxo-7H-pteridin-2-yl]amino]-3-methoxy-N-[2-[2-[5-methoxy-N-methyl-4-[1[4-(1-methylindol-3-yl)pyrimidin-2-yl]amino]-2-(prop-2-enoylamino)anilino]ethyl-methyl-amino]ethyl]benzamide (8.61 mg, 18% yield). ¹H NMR (400 MHz, MeOD) δ8.44-8.59 (m, 1H), 8.22-8.32 (m, 2H), 8.02-8.14 (m, 1H), 7.09-7.49 (m, 8H), 6.84-6.94 (m, 1H), 6.33-6.57 (m, 2H), 5.74-5.89 (m, 1H), 4.03-4.16 (m, 2H), 3.84-4.01 (m, 10H), 3.50-3.69 (m, 2H), 3.16-3.25 (m, 5H), 2.83-3.04 (m, 3H), 2.50-2.77 (m, 7H), 2.06-2.16 (m, 1H), 1.51-1.85 (m, 10H), 0.72-0.75 (m, J=7.46 Hz, 3H). LC-MS: MS (ES′): RT=1.553 min, m/z=936.4 [M+H⁺]; LCMS method: 25.

### Example 100—Synthesis of Compound I-116

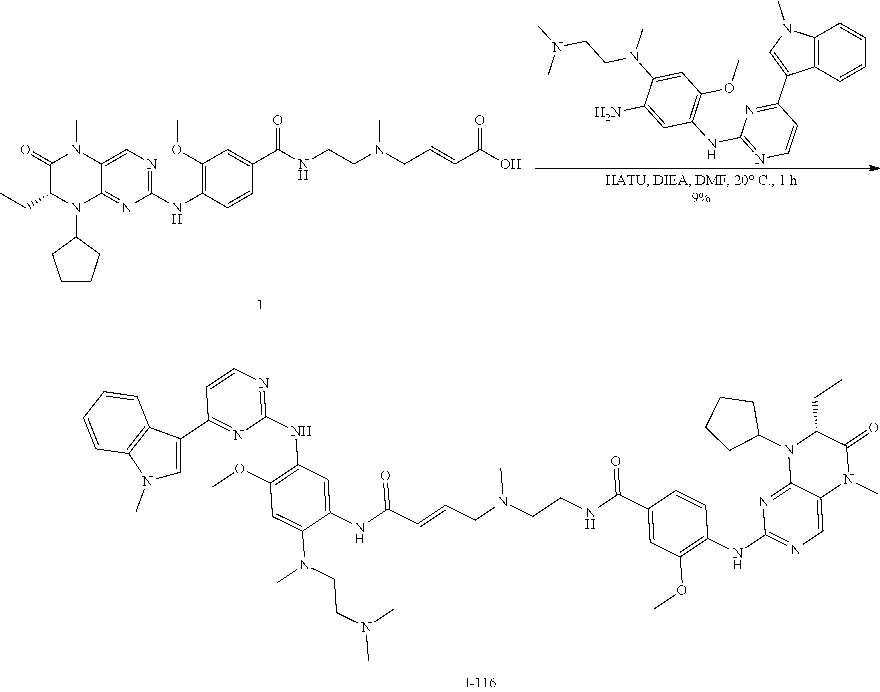 [SMILES: CC[C@@H]1C(=O)N(C)c2cnc(Nc3ccc(C(=O)NCCN(C)C/C=C/C(=O)O)cc3OC)nc2N1C1CCCC1 ; COc1cc(N(C)CCN(C)C)c(N)cc1Nc1nccc(-c2cn(C)c3ccccc23)n1 ; CC[C@@H]1C(=O)N(C)c2cnc(Nc3ccc(C(=O)NCCN(C)C/C=C/C(=O)Nc4cc(Nc5nccc(-c6cn(C)c7ccccc67)n5)c(OC)cc4N(C)CCN(C)C)cc3OC)nc2N1C1CCCC1]

General Information: Synthetic route of compound 2 was reported in Catalysis Letters, 2019, vol. 149(9), p. 2607-2613

To a solution of N₁-[2-(dimethylamino)ethyl]-5-methoxy-N1-methyl-N4-[4-(1-methylindol-3-yl)pyrimidin-2-yl]benzene-1,2,4-triamine (50 mg, 112 μmol, 1 equiv) and (E)-4-[2-[[4-[[(7R)-8-cyclopentyl-7-ethyl-5-methyl-6-oxo-7H-pteridin-2-yl]amino]-3-methoxy-benzoyl]amino]ethyl-methyl-amino]but-2-enoic acid (63 mg, 112 μmol, 1 equiv) in DMF (1 mL) was added DIEA (43 mg, 336 μmol, 3 equiv) and HATU (51 mg, 134 μmol, 1.2 equiv). The mixture was stirred at 20° C. for 1 h and concentrated. The residue was purified by prep-HPLC (column: Waters Xbridge 150*25 mm*5 m; mobile phase: [water (NH₄HCO₃)-ACN]; B%: 53%-83%, 8 min) to give 4-[[(7R)-8-cyclopentyl-7-ethyl-5-methyl-6-oxo-7H-pteridin-2-yl]amino]-N-[2-[[(E)-4-1[2-[2-(dimethylamino)ethyl-methyl-amino]-4-methoxy-5-[[4-(1-methylindol-3-yl)pyrimidin-2-yl]amino]anilino]-4-oxo-but-2-enyl]-methyl-amino]ethyl]-3-methoxy-benzamide (10 mg, 9% yield). ¹H NMR (400 MHz, MeOD): δ 9.41 (s, 1H), 8.8-8.6 (m, 1H), 8.30 (d, 1H, J=8.4 Hz), 8.17 (d, 1H, J=5.2 Hz), 7.96 (s, 1H), 7.48 (s, 1H), 7.4-7.1 (m, 4H), 7.0-6.8 (m, 3H), 4.2-3.8 (m, 6H), 3.78 (s, 3H), 3.50 (s, 4H), 3.2-2.9 (m, 7H), 2.73 (s, 3H), 2.6-2.0 (m, 15H), 1.9-1.2 (m, 10H), 0.74 (t, 3H, J=7.2 Hz). LC-MS: MS (ES′): RT=1.791 min, m/z=993.4 [M+H⁺]; LCMS method: 10.

### Example 101—Synthesis of Compound I-117

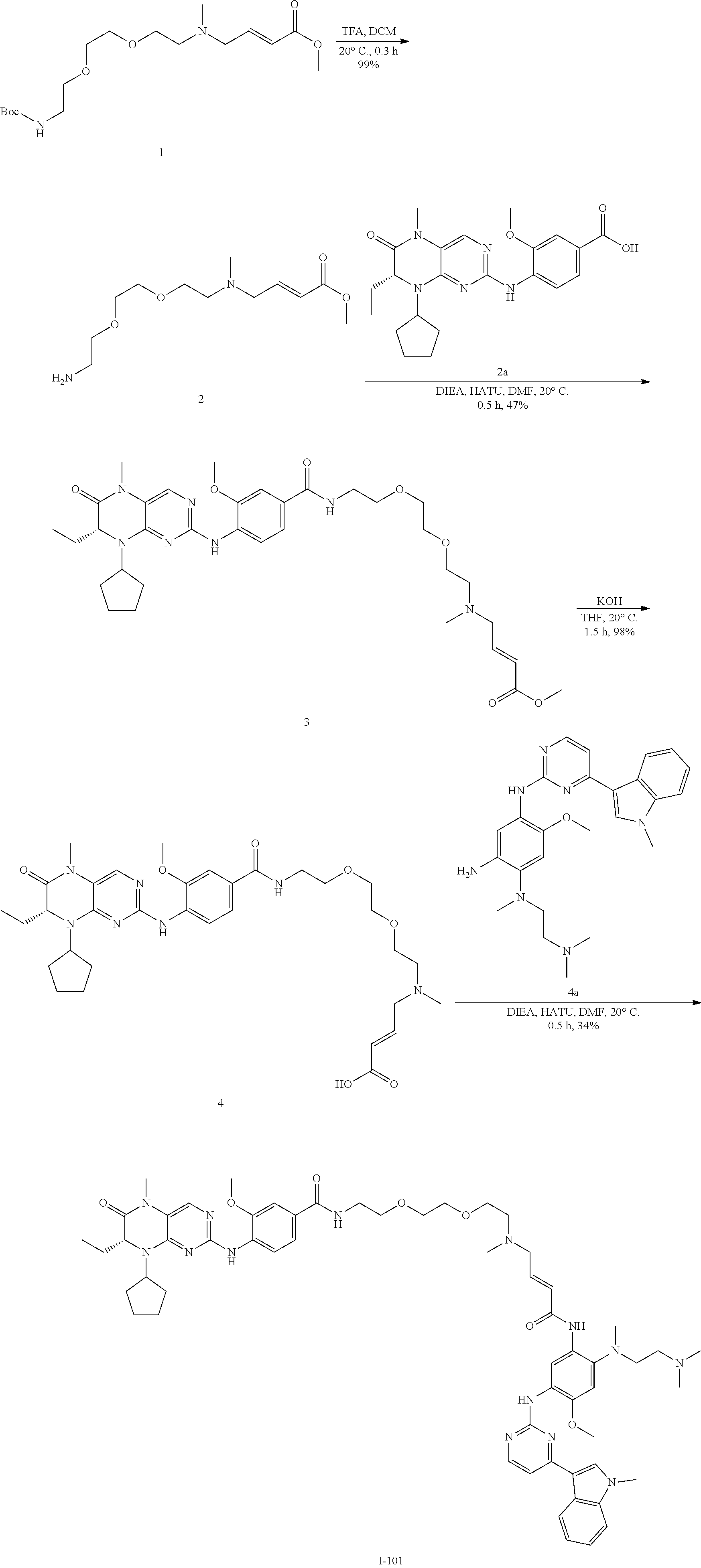

General Information: Synthetic route of compound 2a is a known compound from WO2017/24317, 2017, A2. Synthetic route of compound 4a is a known compound from Catalysis Letters, 2019, vol. 149, # 9, p. 2607-2613.

Step 1. To a solution of methyl (E)-4-[2-[2-[2-(tert-butoxycarbonylamino) ethoxy]ethoxy]ethyl-methyl-amino]but-2-enoate (300 mg, 832 μmol, 1.0 equiv) in DCM (5 mL) was added TFA (4 mL), the mixture was stirred at 20° C. for 0.3 h and concentrated. The product was used directly for next step.

Step 2. To a solution of methyl (E)-4-[2-[2-(2-aminoethoxy) ethoxy]ethyl-methyl-amino]but-2-enoate (290 mg, 776 μmol, 1.1 equiv, TFA salt), 4-[[(7R)-8-cyclopentyl-7-ethyl-5-methyl-6-oxo-7H-pteridin-2-yl]amino]-3-methoxy-benzoic acid (300 mg, 705 μmol, 1.0 equiv) in DMF (4 mL) was added HATU (321 mg, 846 μmol, 1.2 equiv) and DIEA (273 mg, 2.1 mmol, 3.0 equiv). The mixture was stirred at 20° C. for 0.5 h and concentrated. It was purified by prep-HPLC(column: Waters Xbridge C18 150*50 mm*10%m; mobile phase: [water(NH₄HCO₃)-ACN]; B%: 36%-66%, 11 min) to give methyl (E)-4-[2-[2-[2-[[4-[[(7R)-8-cyclopentyl-7-ethyl-5-methyl-6-oxo-7H-pteridin-2-yl]amino]-3-methoxybenzoyl]amino]ethoxy]ethoxy]ethyl-methyl-amino]but-2-enoate (220 mg, 47% yield).

Step 3. To a solution of methyl (E)-4-[2-[2-[2-[ [4-[[(7R)-8-cyclopentyl-7-ethyl-5-methyl-6-oxo-7H-pteridin-2-yl]amino]-3-methoxybenzoyl]amino]ethoxy]ethoxy]ethyl-methyl-amino]but-2-enoate (220 mg, 329 μmol, 1.0 equiv) in THF (5 mL) was added KOH (0.5 M, 1.5 mL, 2.3 equiv). The mixture was stirred at 20° C. for 1.5 h and purified by prep-HPLC(column: Waters Xbridge BEH C18 150*25 mm*5 m; mobile phase: [water(NH₄HCO₃)—ACN]; B%: 19%-49%, 9 min) to give (E)-4-[2-[2-[2-[[4-[[(7R)-8-cyclopentyl-7-ethyl-5-methyl-6-oxo-7H-pteridin-2-yl]amino]-3-methoxybenzoyl]amino]ethoxy]ethoxy]ethyl-methyl-amino]but-2-enoic acid (210 mg, 97% yield).

Step 4. To a solution of(E)-4-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethyl-methyl-amino]but-2-enoic acid (70 mg, 107 μmol, 1.0 equiv),N1-[2-(dimethylamino)ethyl]-5-methoxy-N1-methyl-N4-[4-(1-methylindol-3-yl)pyrimidin-2-yl]benzene-1,2,4-triamine (48 mg, 107 μmol, 1.0 equiv) in DMF (2 mL) was added DIEA (42 mg, 321 μmol, 3.0 equiv) and HATU (49 mg, 128 μmol, 1.2 equiv). The mixture was stirred at 20° C. for 0.5 h and concentrated. The residue was purified by prep-HPLC (column: Unisil 3-100 C18 Ultra 150*50 mm*3 m; mobile phase: [water(FA)-ACN]; B%: 1%-30%, 10 min) to give 4-[[(7R)-8-cyclopentyl-7-ethyl-5-methyl-6-oxo-7H-pteridin-2-yl]amino]-N-[2-[2-[2-1[[(E)-4-1[2-[2-(dimethylamino)ethyl-methyl-amino]-4-methoxy-5-1[[4-(1-methylindol-3-yl)pyrimidin-2-yl]amino]anilino]-4-oxo-but-2-enyl]-methyl-amino]ethoxy]ethoxy]ethyl]-3-methoxy-benzamide (40.3 mg, 34% yield). ¹H NMR (400 MHz, MeOD) δ8.90-8.81 (m, 1H), 8.58-8.49 (m, 1H), 8.41-8.34 (m, 1H), 8.29-8.17 (m, 3H), 7.72-7.61 (m, 1H), 7.44-7.32 (m, 3H), 7.25-7.19 (m, 1H), 7.18-7.12 (m, 2H), 7.06-6.94 (m, 2H), 6.50-6.37 (m, 1H), 4.40-4.33 (m, 1H), 4.23-4.19 (m, 1H), 4.01 (s, 3H), 3.93-3.90 (m, 3H), 3.88-3.84 (m, 3H), 3.74-3.66 (m, 8H), 3.61-3.55 (m, 2H), 3.39 (s, 4H), 3.30-3.27 (m, 3H), 3.13-3.02 (m, 2H), 2.80-2.69 (m, 11H), 2.43-2.35 (m, 3H), 2.20-2.10 (m, 1H), 1.94-1.77 (m, 5H), 1.75-1.62 (m, 4H), 0.85-0.75 (m, 3H). LC-MS: MS (ES⁺): RT=1.932 min, m/z=1081.5 [M+H⁺]; LCMS method: 25.

### Example 102—Synthesis of Compound I-118

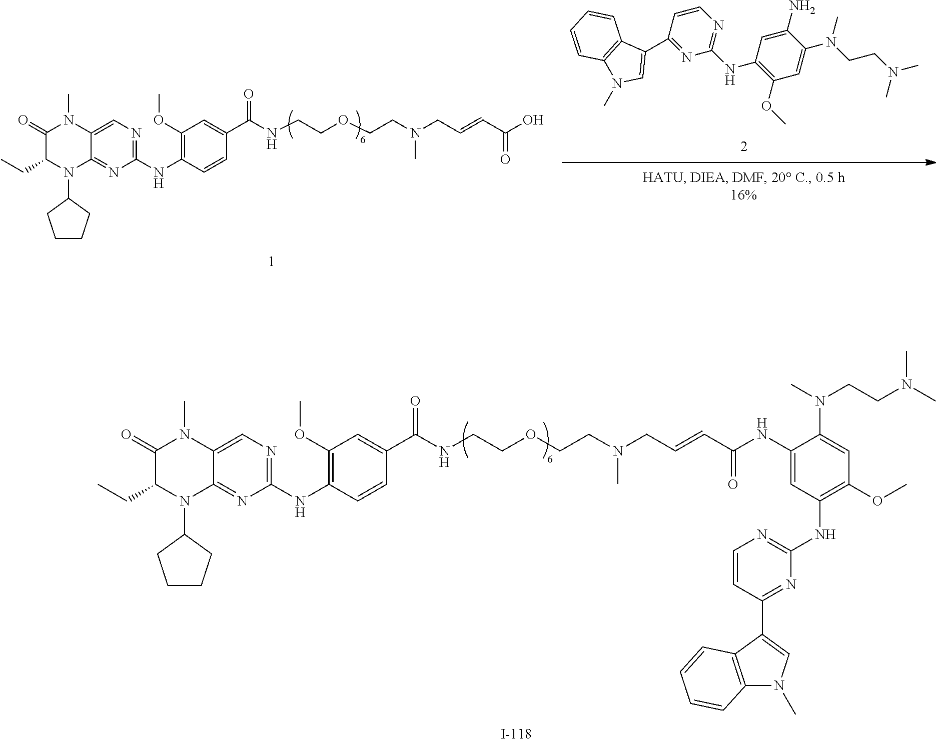

General Information: Compound 2 is a known compound from Catalysis Letters, 2019, vol. 149, # 9, p. 2607-2613.

Step 1. To a solution of (E)-4-[2-[2-[2-[2-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethoxy]ethoxy]ethyl-methyl-amino]but-2-enoic acid (50 mg, 60 μmol, 1.0 equiv), N1-[2-(dimethylamino)ethyl]-5-methoxy-N1-methyl-N4-[4-(1-methylindol-3-yl)pyrimidin-2-yl]benzene-1,2,4-triamine (26 mg, 60 μmol, 1.0 equiv) in DMF (2 mL) was added HATU (27 mg, 72 μmol, 1.2 equiv) and DIEA (23 mg, 180 μmol, 3.0 equiv). The mixture was stirred at 20° C. for 0.5h. The solution was purified by prep-HPLC (column: Unisil 3-100 C18 Ultra 150*50 mm*3 m; mobile phase: [water(FA)-ACN]; B%: 2%-32%, 10 min) to give 4-[[(7R)-8-cyclopentyl-7-ethyl-5-methyl-6-oxo-7H-pteridin-2-yl]amino]-N-[2-[2-[2-[2-[2-[2-[2-[[(E)-4-[2-[2-(dimethylamino)ethyl-methyl-amino]-4-methoxy-5-[[4-(1-methylindol-3-yl)pyrimidin-2-yl]amino]anilino]-4-oxo-but-2-enyl]-methyl-amino]ethoxy]ethoxy]ethoxy]ethoxy]ethoxy]ethoxy]ethyl]-3-methoxy-benzamide (12.4 mg, 16% yield). ¹H NMR (400 MHz, CD₃OD): δ8.73 (s, 1H), 8.50-8.42 (m, 3H), 8.33 (d, J=8.0 Hz, 1H), 8.25 (d, J=5.6 Hz, 1H), 8.18 (s, 1H), 7.74 (s, 1H), 7.48-7.43 (m, 3H), 7.27-7.22 (m, 1H), 7.20-7.15 (m, 2H), 7.08-6.95 (m, 2H), 6.45 (d, J=15.2 Hz, 1H), 4.02 (s, 3H), 3.97 (s, 3H), 3.90 (s, 3H), 3.70-3.44 (m, 32H), 3.24-3.08 (m, 3H), 2.88-2.82 (m, 8H), 2.74 (s, 3H), 2.46 (s, 3H), 2.17-2.14 (m, 1H), 1.98-1.96 (m, 1H), 1.90-1.68 (m, 8H), 0.84 (t, J=7.6 Hz, 3H). LC-MS: MS (ES⁺): RT=1.999 min, m/z=629.4 [M+H⁺]/2; LCMS method: 10

### Example 103—Synthesis of Compound I-119

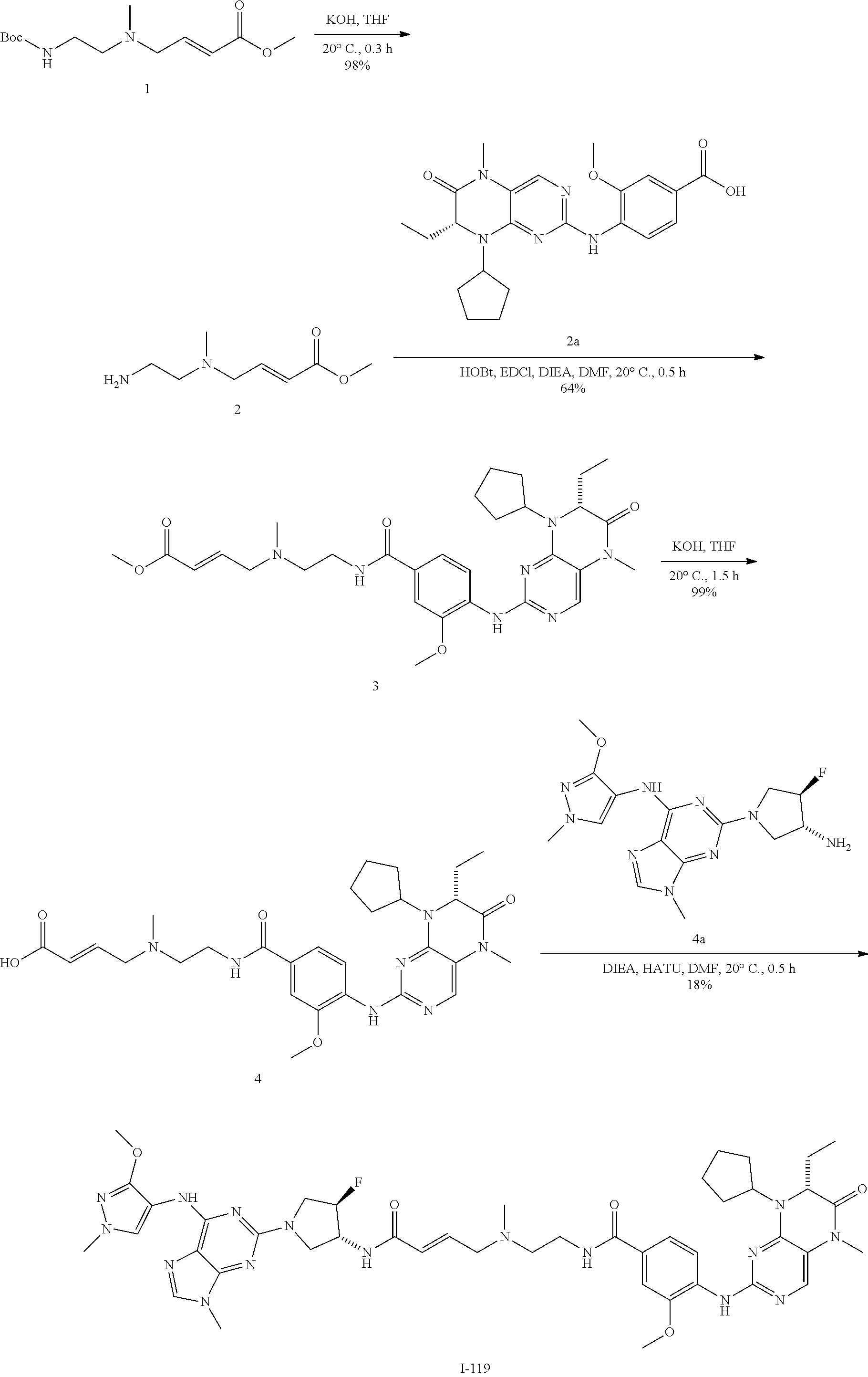

Step 1. To a solution of methyl (E)-4-[2-(tert-butoxycarbonylamino)ethyl-methyl-amino]but-2-enoate (300 mg, 1.1 mmol, 1 equiv)in DCM (5 mL) was added TFA (3.08 g, 27. mmol, 24.5 equiv), the mixture was stirred at 20° C. for 0.3 h. The mixture was concentrated to give methyl (E)-4-[2-aminoethyl(methyl)amino]but-2-enoate (310 mg, 98% yield).

Step 2. To a solution of 4-[[(7R)-8-cyclopentyl-7-ethyl-5-methyl-6-oxo-7H-pteridin-2-yl]amino]-3-methoxy-benzoic acid (460 mg, 1.08 mmol, 1 equiv),methyl (E)-4-[2-aminoethyl(methyl)amino]but-2-enoate (310 mg, 1.08 mmol, 1 equiv, TFA salt)in DMF (8 mL) was added EDCI (311 mg, 1.62 mmol, 1.5 equiv) and DIEA (419 mg, 3.25 mmol, 3 equiv), HOBt (219 mg, 1.62 mmol, 1.5 equiv). The mixture was stirred at 20° C. for 0.5 h and concentrated. It was purified by prep-HPLC(column: Phenomenex luna C18 150*40 mm*15 m; mobile phase: [water (FA)-ACN]; B%: 3%-33%, 10 min) to give methyl (E)-4-[2-[[4-[[(7R)-8-cyclopentyl-7-ethyl-5-methyl-6-oxo-7H-pteridin-2-yl]amino]-3-methoxy-benzoyl]amino]ethylmethyl-amino]but-2-enoate (400 mg, 63% yield) as a yellow solid.

Step 3. To a solution of methyl (E)-4-[2-[[4-[[(7R)-8-cyclopentyl-7-ethyl-5-methyl-6-oxo-7H-pteridin-2-yl]amino]-3-methoxybenzoyl]amino]ethyl-methyl-amino]but-2-enoate (350 mg, 603 μmol, 1 equiv) in THF (5 mL) was added KOH (0.5 M, 1.5 mL, 1.24 equiv). The mixture was stirred at 20° C. for 1.5 h. The solvent was removed to give a residue. The residue was dissolved in water (20 mL) and the mixture was extracted with EtOAc (30 mL×2). Then the aqueous phase was collected and acidized by 2N HCl to adjust the PH to 7. And the mixture was extracted with EtOAc (50 mL×3). The combined organic layers were washed by brine (30 mL), dried over Na₂SO₄, filtered and concentrated to give the desired product. (E)-4-[2-[[4-[[(7R)-8-cyclopentyl-7-ethyl-5-methyl-6-oxo-7H-pteridin-2-yl]amino]-3-methoxy-benzoyl]amino]ethyl-methylamino]but-2-enoic acid (340 mg, 99% yield) as a yellow solid.

Step 4. To a solution of (E)-4-[2-[[4-[[(7R)-8-cyclopentyl-7-ethyl-5-methyl-6-oxo-7H-pteridin-2-yl]amino]-3-methoxy-benzoyl]amino]ethyl-methyl-amino]but-2-enoic acid (70 mg, 123 μmol, 1 equiv) in DMF (3 mL) was added DIEA (79 mg, 618 μmol, 5 equiv), HATU (56 mg, 148 μmol, 1.2 equiv) and 2-[(3R,4R)-3-amino-4-fluoro-pyrrolidin-1-yl]-N-(3-methoxy-1-methyl-pyrazol-4-yl)-9-methyl-purin-6-amine (58 mg, 123 μmol, 1.0 equiv, TFA salt). The mixture was stirred at 20° C. for 0.5 h. The mixture was concentrated and purified by prep-HPLC(column: Phenomenex Synergi C₁₈ 150*25 mm*10 μm; mobile phase: [water (FA)-ACN]; B%: 5%-35%, 10 min) to give 4-[l[(7R)-8-cyclopentyl-7-ethyl-5-methyl-6-oxo-7H-pteridin-2-yl]amino]-N—[2-[ [(E)-4-[ [(3R,4R)-4-fluoro-1-[6-[(3-methoxy-1-methyl-pyrazol-4-yl)amino]-9-methyl-purin-2-yl]pyrrolidin-3-yl]amino]-4-oxo-but-2-enyl]-methyl-amino]ethyl]-3-methoxy-benzamide (20 mg, 18% yield). ¹H NMR (400 MHz, MeOD): δ=8.33-8.25 (m, 1H), 8.34-8.22 (m, 1H), 7.96 (s, 1H), 7.74 (s, 1H), 7.68 (s, 1H), 7.13-6.84 (m, 2H), 5.23-5.06 (m, 1H), 4.60-4.21 (m, 6H), 3.96-3.64 (m, 15H), 3.34 (d, J=5.2 Hz, 3H), 2.96-2.72 (m, 3H), 2.65-2.44 (m, 1H), 2.37-2.21 (m, 4H), 2.19-1.68 (m, 1OH), 0.90-0.80 (m, 3H). LC-MS: MS (ES′): RT=1.934 min, m/z=909.4 [M+H⁺]; LCMS method: 10.

### Example 104—Synthesis of Compound I-120

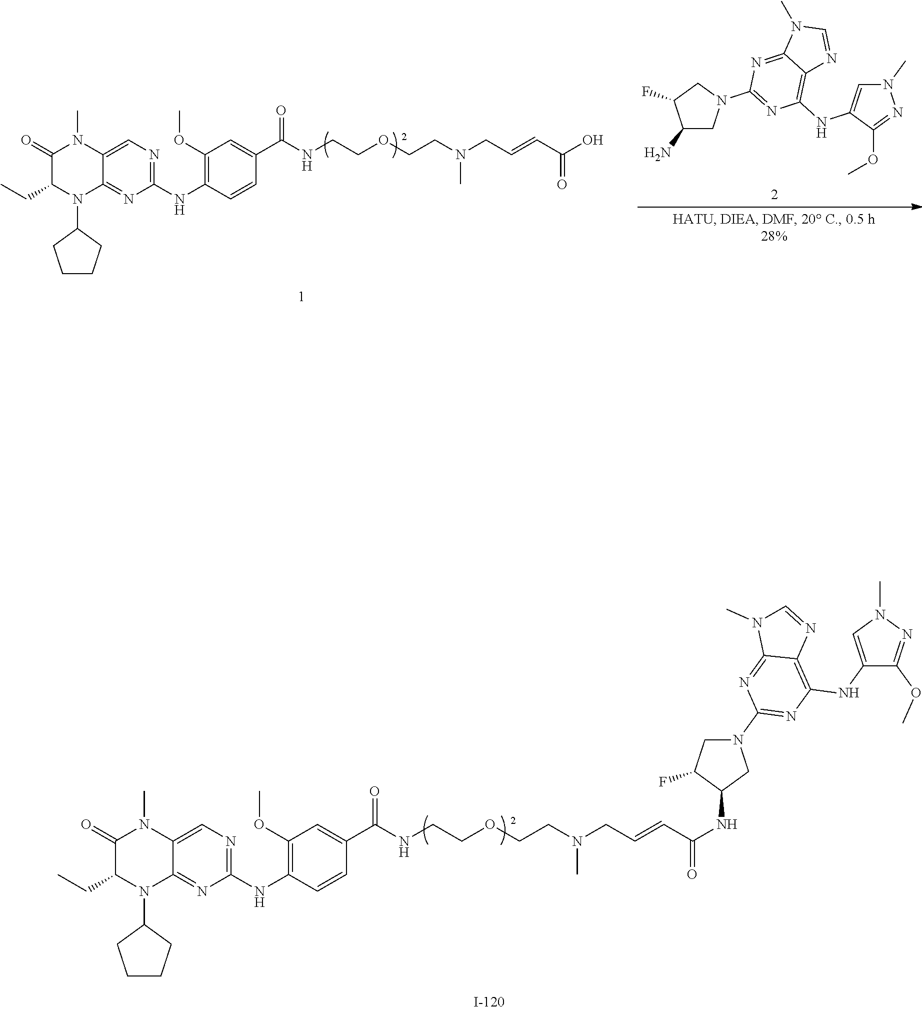

To a solution of 2-[(3R,4R)-3-amino-4-fluoro-pyrrolidin-1-yl]-N-(3-methoxy-1-methyl-pyrazol-4-yl)-9-methyl-purin-6-amine (50 mg, 107 μmol, 1.0 equiv, TFA salt), (E)-4-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethyl-methyl-amino]but-2-enoic acid (70 mg, 107 μmol, 1.0 equiv) in DMF (3 mL) was added HATU (49 mg, 128 μmol, 1.2 equiv) and DIEA (41 mg, 321 μmol, 3.0 equiv). The mixture was stirred at 20° C. for 0.5h. The solution was purified by prep-HPLC (column: Unisil 3-100 C18 Ultra 150*50 mm*3 m; mobile phase: [water(FA)-ACN]; B%: 5%-35%, 10 min) to give 4-[[(7R)-8-cyclopentyl-7-ethyl-5-methyl-6-oxo-7H-pteridin-2-yl]amino]-N-[2-[2-[2-[[(E)-4-1[[(3R,4R)-4-fluoro-1-[6-1[(3-methoxy-1-methyl-pyrazol-4-yl)amino]-9-methyl-purin-2-yl]pyrrolidin-3-yl]amino]-4-oxo-but-2-enyl]-methylamino]ethoxy]ethoxy]ethyl]-3-methoxy-benzamide (31 mg, 28% yield). ¹H NMR (400 MHz, CD₃OD): δ8.43 (d, J=8.8 Hz, 1H), 7.96 (s, 1H), 7.84-7.63 (m, 2H), 7.51-7.35 (m, 2H), 6.91-6.78 (m, 1H), 6.26 (d, J=14.8 Hz, 1H), 5.33-4.94 (m, 3H), 4.51-4.43 (m, 1H), 4.30-4.27 (m, 1H), 3.97 (d, J=1.6 Hz, 8H), 3.85 (t, J=11.6 Hz, 2H), 3.74 (s, 3H), 3.71-3.35 (m, 17H), 2.86-2.82 (m, 2H), 2.45 (s, 3H), 2.17 (d,J=9.2 Hz, 1H), 2.03-2.00 (m, 1H), 1.95-1.72 (m, 8H), 0.86 (t, J=7.6 Hz, 3H). LC-MS: MS (ES′): RT=1.998 min, m/z=997.4 [M+H⁺]; LCMS method: 10.

### Example 105—Synthesis of Compound I-121

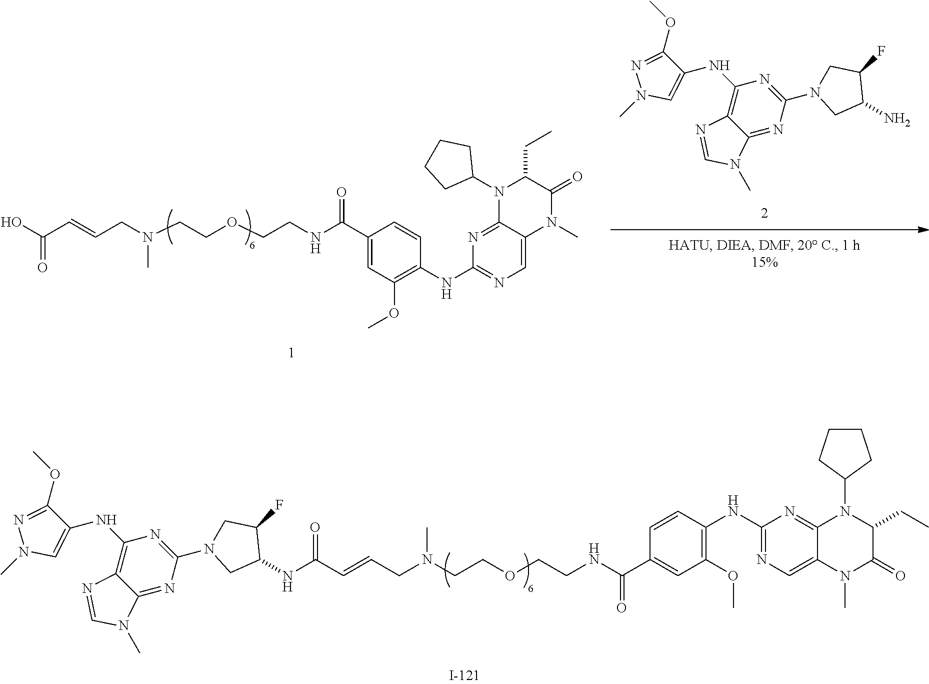

To a solution of (E)-4-[2-[2-[2-[2-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethoxy]ethoxy]ethyl-methyl-amino]but-2-enoic acid (60 mg, 72 μmol, 1 equiv) and 2-[(3R,4R)-3-amino-4-fluoro-pyrrolidin-1-yl]-N-(3-methoxy-1-methyl-pyrazol-4-yl)-9-methyl-purin-6-amine (34 mg, 72 μmol, 1 equiv, TFA salt) in DMF (1 mL) was added DIEA (46 mg, 361 μmol, 5 equiv) and HATU (32 mg, 86 μmol, 1.2 equiv). The mixture was stirred at 20° C. for 1 h and concentrated. The residue was purified by prep-HPLC (column: Phenomenex luna C18 250*50 mm*15 m; mobile phase: [water(FA)-ACN]; B %: 16%-46%, 10 min) to give 4-[[(7R)-8-cyclopentyl-7-ethyl-5-methyl-6-oxo-7H-pteridin-2-yl]amino]-N-[2-[2-[2-[2-[2-[2-[2-[[(E)-4-[1[(3R,4R)-4-fluoro-1-1[6-1[(3-methoxy-1-methyl-pyrazol-4-yl)amino]-9-methyl-purin-2-yl]pyrrolidin-3-yl]amino]-4-oxo-but-2-enyl]-methyl-amino]ethoxy]ethoxy]ethoxy]ethoxy]ethoxy]ethoxy]ethyl]-3-methoxy-benzamide (13 mg, 15% yield). ¹H NMR (400 MHz, MeOD): δ8.43 (d, 1H, J=8.4 Hz), 8.36 (s, 1H), 7.95 (s, 1H), 7.74 (d, 2H, J=17.2 Hz), 7.6-7.3 (m, 2H), 6.9-6.7 (m, 1H), 6.38 (d, 1H, J=14.8 Hz), 5.3-5.1 (m, 1H), 4.7-4.4 (m, 5H), 4.29 (dd, 1H, J=3.6, 7.6 Hz), 4.1-3.4 (m, 42H), 3.35 (s, 3H), 3.15 (s, 2H), 2.75 (s, 3H), 2.3-1.7 (m, 10H), 0.8-0.9 (m, 3H). LC-MS: MS (ES⁺): RT=1.791 min, m/z=587.3 [M/2+H⁺]; LCMS method: 10.

### Example 106—Synthesis of Compound I-122

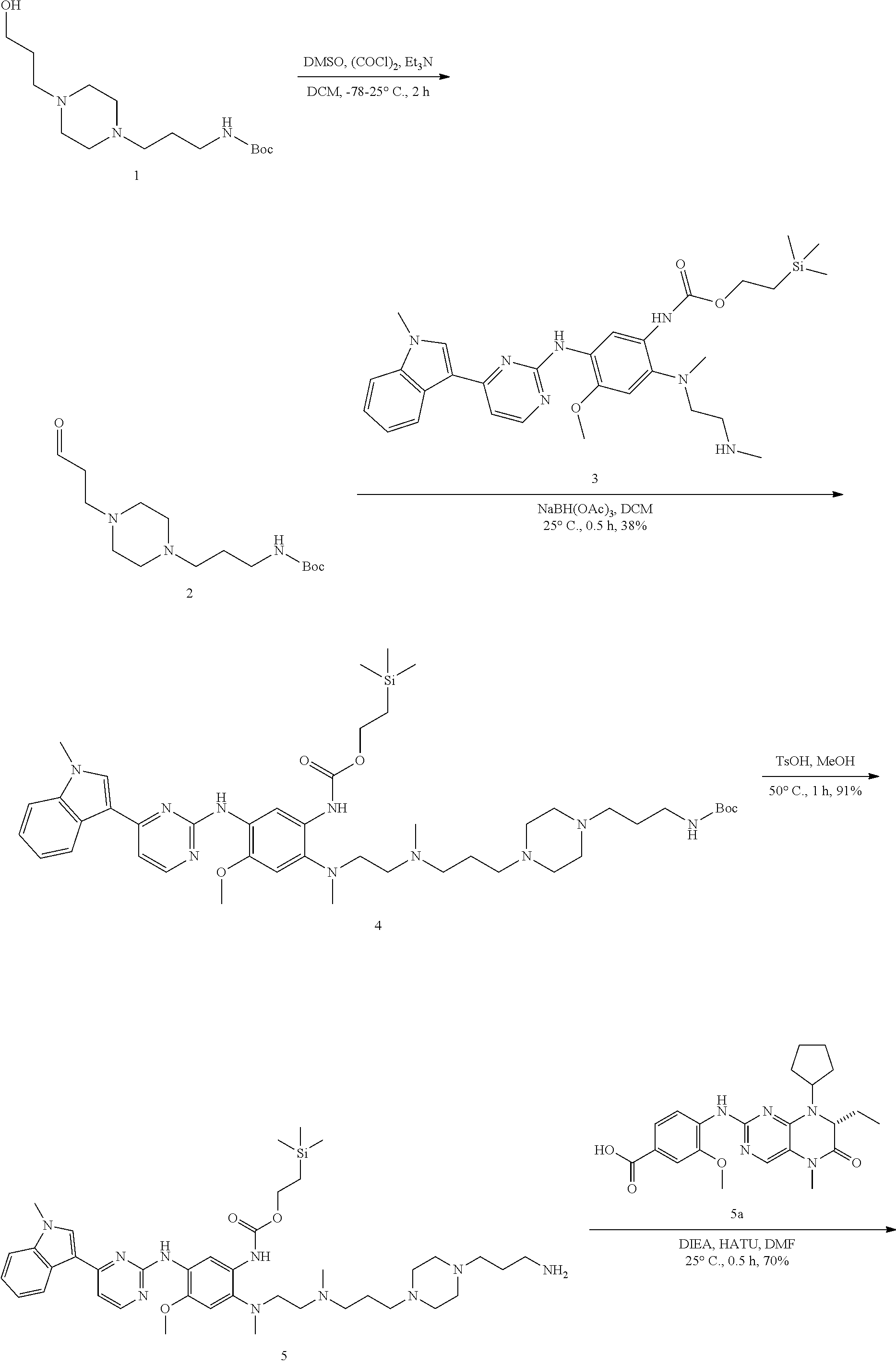 [SMILES: CC(C)(C)OC(=O)NCCCN1CCN(CCCO)CC1 ; CC(C)(C)OC(=O)NCCCN1CCN(CCC=O)CC1 ; CNCCN(C)c1cc(OC)c(Nc2nccc(-c3cn(C)c4ccccc34)n2)cc1NC(=O)OCC[Si](C)(C)C ; COc1cc(N(C)CCN(C)CCCN2CCN(CCCNC(=O)OC(C)(C)C)CC2)c(NC(=O)OCC[Si](C)(C)C)cc1Nc1nccc(-c2cn(C)c3ccccc23)n1 ; COc1cc(N(C)CCN(C)CCCN2CCN(CCCN)CC2)c(NC(=O)OCC[Si](C)(C)C)cc1Nc1nccc(-c2cn(C)c3ccccc23)n1 ; CC[C@@H]1C(=O)N(C)c2cnc(Nc3ccc(C(=O)O)cc3OC)nc2N1C1CCCC1]

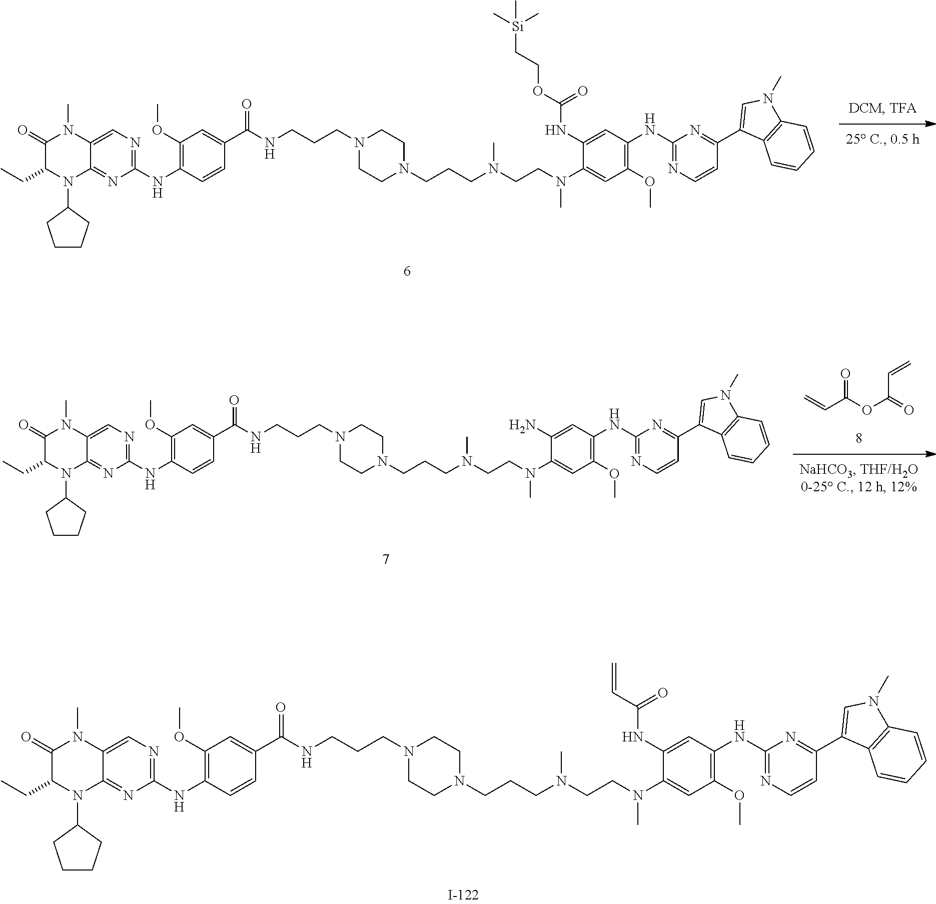

General Information: Compound 5a is a known compound from WO2017/24317, 2017, A2.

Step 1. To a solution of DMSO (518.4 mg, 6.6 mmol, 518.3 μL, 10.0 equiv) in DCM (4 mL) was added (COCl)₂ (336.8 mg, 2.6 mmol, 232.3 μL, 4.0 equiv) in DCM (1 mL) at −70° C. and stirred for 0.5 h. Then tert-butyl N-[3-[4-(3-hydroxypropyl)piperazin-1-yl]propyl]carbamate (0.2 g, 663.2 μmol, 1.0 equiv) in DCM (1 mL) was added and stirred at −70° C. for 0.5 h. Et₃N (1.1 g, 9.9 mmol, 1.9 mL, 15.0 equiv) in DCM (1 mL) was added to the solution and stirred at -70° C. for 0.5 h. The mixture was slowly warmed to 25° C. and stirred at 25° C. for 0.5 h. Compound tert-butyl N-[3-[4-(3-oxopropyl) piperazin-1-yl]propyl]carbamate (198 mg, 99% yield) was obtained as a colorless liquid in DCM and used directly for the next step.

Step 2. To a solution of 2-trimethylsilylethyl N-[4-methoxy-5-[[4-(1-methylindol-3-yl) pyrimidin-2-yl]amino]-2-[methyl-[2-(methylamino) ethyl]amino]phenyl]carbamate (242.3 mg, 420.8 μmol, 0.7 equiv) in DCM (5 mL) was added NaBH(OAc)₃ (2.5 g, 12.2 mmol, 20.0 equiv) and tert-butyl N-[3-[4-(3-oxopropyl) piperazin-1-yl]propyl]carbamate (0.18 g, 601.9 μmol, 1.0 equiv), and then it was stirred at 25° C. for 0.5 h. The residue was diluted with H₂O (20 mL) and extracted with DCM (30 mL×3), dried over Na₂SO₄, filtered and concentrated under reduced pressure to give a residue. The residue was purified by prep-HPLC (column: Phenomenex luna C18 250*50 mm*15 m; mobile phase: [water (FA)-ACN]; B%: 20%-50%, 10 min) to give compound 2-trimethylsilylethyl N-[2-[2-[3-[4-[3-(tert-butoxycarbonylamino)propyl]piperazin-1-yl]propyl-methyl-amino]ethyl-methyl-amino]-4-methoxy-5-[[4-(1-methylindol-3-yl)pyrimidin-2-yl]amino]phenyl]carbamate (0.2 g, 38% yield) as a yellow oil.

Step 3. To a solution of 2-trimethylsilylethyl N-[2-[2-[3-[4-[3-(tert-butoxy carbonylamino)propyl]piperazin-1-yl]propyl-methyl-amino]ethyl-methyl-amino]-4-methoxy-5-[[4-(1-methylindol-3-yl)pyrimidin-2-yl]amino]phenyl]carbamate (150 mg, 174.8 μmol, 1.0 equiv) in MeOH (2 mL) was added TsOH (135.9 mg, 785.6 μmol, 4.5 equiv). The mixture was stirred at 50° C. for 1 h. The reaction mixture was concentrated under reduced pressure to give compound 2-trimethylsilyl ethyl N-[2-[2-[3-[4-(3-aminopropyl)piperazin-1-yl]propyl-methyl-amino]ethyl-methyl-amino]-4-methoxy-5-[[4-(1-methylindol-3-yl)pyrimidin-2-yl]amino]phenyl]carbamate (0.12 g, 91% yield) as a yellow oil.

Step 4. To a solution of 2-trimethylsilylethyl N-[2-[2-[3-[4-(3-aminopropyl)piperazin-1-yl]propyl-methyl-amino]ethyl-methyl-amino]-4-methoxy-5-[[4-(1-methylindol-3-yl)pyrimidin-2-yl]amino]phenyl]carbamate (140 mg, 184.4 μmol, 1.0 equiv), 4-[(8-cyclopentyl-7-ethyl-5-methyl-6-oxo-7H-pteridin-2-yl)amino]-3-methoxy-benzoic acid (156.9 mg, 368.8 μmol, 2.0 equiv) in DMF (0.5 mL) was added DIEA (71.5 mg, 553.3 μmol, 96.8 μL, 3.0 equiv) and HATU (77.4 mg, 202.8 μmol, 1.1 equiv). The mixture was stirred at 25° C. for 0.5 h. The residue was purified by prep-HPLC (column:Phenomenex C18 75*30 mm*3 m; mobile phase: [water(FA)-ACN]; B%: 18%-48%, 7 min) to give compound 2-trimethylsilylethyl N-[2-[2-[3-[4-[33-[ [4-[(8-cyclopentyl-7-ethyl-5-methyl-6-oxo-7H-pteridin-2-yl)amino]-3-methoxy-benzoyl]amino]propyl]piperazin-1-yl]propyl-methyl-amino]ethyl-methyl-amino]-4-methoxy-5-[[4-(1-methylindol-3-yl)pyrimidin-2-yl]amino]phenyl]carbamate (150 mg, 70% yield) as a yellow solid. LC-MS: MS (ES⁺): RT=0.743 min, m/z=1165.4 [M+H]+.

Step 5. To a solution of 2-trimethylsilylethyl N-[2-[2-[3-[4-[3-[[4-[(8-cyclopentyl-7-ethyl-5-methyl-6-oxo-7H-pteridin-2-yl)amino]-3-methoxy-benzoyl]amino]propyl]piperazin-1-yl]propyl-methyl-amino]ethyl-methyl-amino]-4-methoxy-5-[[4-(1-methylindol-3-yl)pyrimidin-2-yl]amino]phenyl]carbamate (0.15 g, 128.9 μmol, 1.0 equiv) in DCM (1 mL) was added TFA (0.5 mL), and then it was stirred at 25° C. for 0.5 h. The reaction mixture was concentrated under reduced pressure to give compound N-[3-[4-[3-[2-[2-amino-5-methoxy-N-methyl-4-[[4-(1-methylindol-3-yl)pyrimidin-2-yl]amino]anilino]ethyl-methyl-amino]propyl]piperazin-1-yl]propyl]-4-[(8-cyclopentyl-7-ethyl-5-methyl-6-oxo-7H-pteridin-2-yl)amino]-3-methoxy-benzamide (0.12 g, 95% yield, TFA) as a yellow solid.

Step 6. To a solution of N-[3-[4-[3-[2-[2-amino-5-methoxy-N-methyl-4-[[4-(1-methylindol-3-yl)pyrimidin-2-yl]amino]anilino]ethyl-methyl-amino]propyl]piperazin-1-yl]propyl]-4-[(8-cyclopentyl-7-ethyl-5-methyl-6-oxo-7H-pteridin-2-yl)amino]-3-methoxy-benzamide (0.12 g, 117.8 μmol, 1.0 equiv) in THF (1 mL) was added NaHCO₃ (190 mg, 2.37 mmol, 20.0 equiv) in H₂O (0.5 mL) and prop-2-enoyl prop-2-enoate (16.8 mg, 129.2 μmol, 1.1 equiv), and then it was stirred at 25° C. for 0.5 h. The residue was diluted with H₂O (30 mL) and extracted with DCM (10 mL×3), dried over Na₂SO₄, filtered and concentrated under reduced pressure to give a residue. The residue was purified by prep-HPLC (column: Phenomenex Synergi C18 150*25 mm*10 m; mobile phase: [water (FA)-ACN]; B%: 5%-35%, 10 min) to give compound 4-[(8-cyclopentyl-7-ethyl-5-methyl-6-oxo-7H-pteridin-2-yl)amino]-3-methoxy-N-[3-[4-[3-[2-[5-methoxy-N-methyl-4-[[4-(1-methylindol-3-yl)pyrimidin-2-yl]amino]-2-(prop-2-enoylamino)anilino]ethyl-methyl-amino]propyl]piperazin-1-yl]propyl]benzamide (16 mg, 12% yield, 91% purity) as a yellow solid. ¹H NMR (400 MHz, CD₃OD): δ8.72 (s, 1H), 8.44-8.53 (m, 2H), 8.33-8.38 (m, 1H), 8.24 (d, J=5.38 Hz, 1H), 8.06-8.16 (m, 1H), 7.80 (s, 1H), 7.39-7.48 (m, 3H), 7.11-7.25 (m, 3H), 6.96 (s, 1H), 6.46-6.55 (m, 2H), 5.84-5.92 (m, 1H), 4.28 (m, 1H), 4.03 (s, 3H), 3.97 (s, 3H), 3.85 (s, 3H), 3.47-3.56 (m, 2H), 3.34 (s, 3H), 3.22-3.25 (m, 4H), 3.00-3.09 (m, 2H), 2.82-2.89 (m, 3H), 2.75 (s, 3H), 2.10-2.43 (m, 12H), 1.51-2.01 (m, 15H), 0.84-0.88 (m, 3H). LC-MS: MS (ES⁺): RT=1.39 min, m/z=1076.6 [M+H⁺]; LCMS method: 25.

### Example 107—Synthesis of Compound I-123

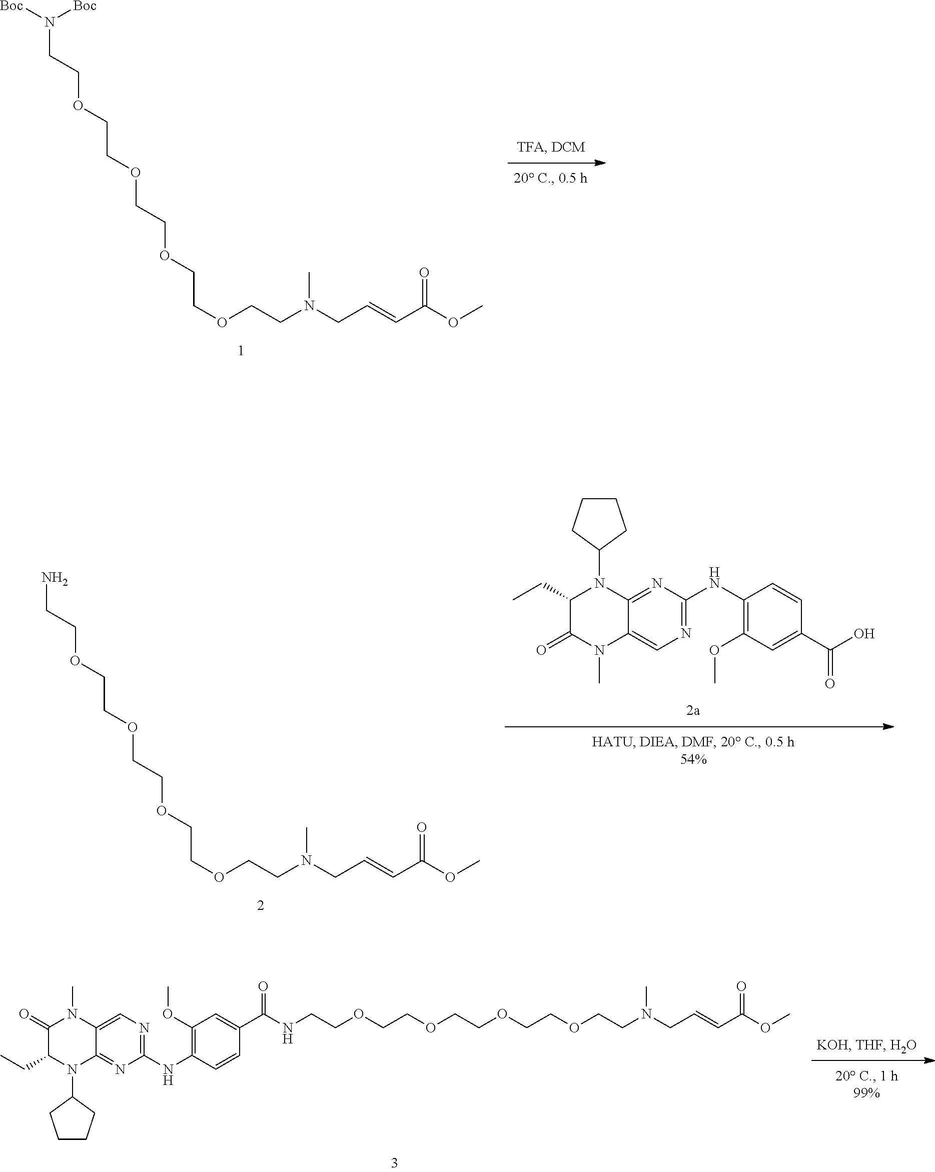 [SMILES: COC(=O)/C=C/CN(C)CCOCCOCCOCCOCCN(C(=O)OC(C)(C)C)C(=O)OC(C)(C)C ; COC(=O)/C=C/CN(C)CCOCCOCCOCCOCCN ; CC[C@H]1C(=O)N(C)c2cnc(Nc3ccc(C(=O)O)cc3OC)nc2N1C1CCCC1 ; CC[C@@H]1C(=O)N(C)c2cnc(Nc3ccc(C(=O)NCCOCCOCCOCCOCCN(C)C/C=C/C(=O)OC)cc3OC)nc2N1C1CCCC1]

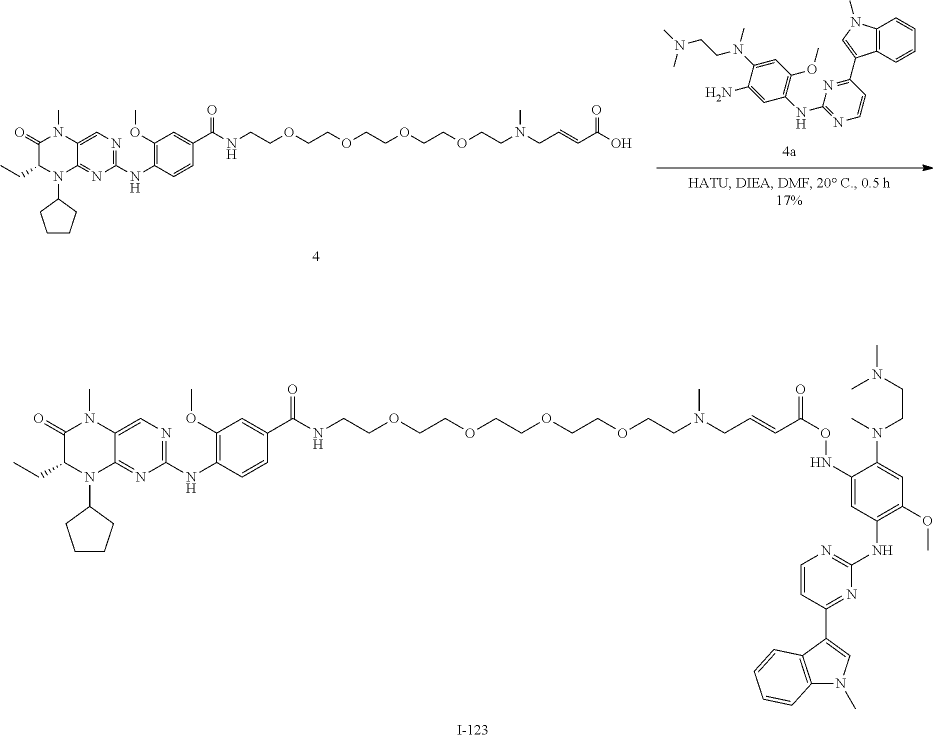 [SMILES: CC[C@@H]1C(=O)N(C)c2cnc(Nc3ccc(C(=O)NCCOCCOCCOCCOCCN(C)C/C=C/C(=O)O)cc3OC)nc2N1C1CCCC1 ; COc1cc(N(C)CCN(C)C)c(N)cc1Nc1nccc(-c2cn(C)c3ccccc23)n1 ; CC[C@@H]1C(=O)N(C)c2cnc(Nc3ccc(C(=O)NCCOCCOCCOCCOCCN(C)C/C=C/C(=O)ONc4cc(Nc5nccc(-c6cn(C)c7ccccc67)n5)c(OC)cc4N(C)CCN(C)C)cc3OC)nc2N1C1CCCC1]

General Information: Compound 4a is a known compound from WO2013/14448, 2013, A1.

Step 1. To a solution of methyl (E)-4-[2-[2-[2-[2-[2-[bis(tert-butoxycarbonyl)amino]ethoxy]ethoxy]ethoxy]ethoxy]ethyl-methyl-amino]but-2-enoate (200 mg, 364 μmol, 1.0 equiv) in DCM (6 mL) was added TFA (2 mL). The mixture was stirred at 20° C. for 0.5 h. The solution was concentrated under reduced pressure to give methyl (E)-4-[2-[2-[2-[2-(2-aminoethoxy) ethoxy]ethoxy]ethoxy]ethyl-methyl-amino]but-2-enoate (168 mg, crude, TFA salt).

Step 2. To a solution of methyl (E)-4-[2-[2-[2-[2-(2-aminoethoxy)ethoxy]ethoxy]ethoxy]ethyl-methyl-amino]but-2-enoate (168 mg, 363 μmol, 1.0 equiv, TFA salt) in DMF (4 mL) was added DIEA (235 mg, 1.82 mmol, 5.0 equiv) and 4-[[(7R)-8-cyclopentyl-7-ethyl-5-methyl-6-oxo-7H-pteridin-2-yl]amino]-3-methoxy-benzoic acid (186 mg, 436 μmol, 1.2 equiv) and HATU (207 mg, 545 μmol, 1.5 equiv). The mixture was stirred at 20° C. for 0.5 h. The mixture was diluted with water (10 mL) and extracted with DCM (3×20 mL). The combined organic layers were dried over sodium sulfate, filtered and concentrated. The residue was purified by prep-TLC (SiO₂, DCM: MeOH=10:1) to give methyl (E)-4-[2-[2-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethyl-methyl-amino]but-2-enoate (150 mg, 54% yield). ¹H NMR (400 MHz, CDCl₃): δ8.78-8.73 (m, 1H), 8.53-8.44 (m, 1H), 8.02-7.97 (m, 1H), 7.78-7.64 (m, 2H), 7.51-7.44 (m, 1H), 4.60-4.49 (m, 1H), 4.33-4.25 (m, 1H), 4.18-3.97 (m, 6H), 3.40-3.32 (m, 3H), 2.25-1.81 (m, 20H), 1.80-1.56 (m, 10H), 1.35-1.21 (m, 6H), 0.93-0.88 (m, 3H).

Step 3. To the solution methyl (E)-4-[2-[2-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethyl-methyl-amino]but-2-enoate (100 mg, 132 μmol, 1.0 equiv) in THF (1 mL) and H₂O (1 mL) was added KOH (37 mg, 661 μmol, 5.0 equiv). The mixture was stirred at 20° C. for 1 h. Adjusted the pH to 5-6 with 1M HCl aqueous at 0° C., and then filtered and concentrated. The residue was triturated with 50 mL DCM:IPA(10:1) at 20° C. for 5 min to give (E)-4-[2-[2-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethyl-methyl-amino]but-2-enoic acid (98 mg, 99% yield).

Step 4. To a solution of (E)-4-[2-1[2-[2-[2-1[2-[1[4-[1[(7R)—-8-cyclopentyl-7-ethyl-5-methyl-6-oxo-7H-pteridin-2-yl]amino]-3-methoxy-benzoyl]amino]ethoxy]ethoxy]ethoxy]ethoxy]ethyl-methyl-amino]but-2-enoic acid (49 mg, 66 μmol, 1.0 equiv) in DMF (1 mL) was added N1-[2-(dimethylamino)ethyl]-5-methoxy-N1-methyl-N4-[4-(1-methylindol-3-yl)pyrim idin-2-yl]benzene-1,2,4-triamine (29 mg, 66 μmol, 1.0 equiv) DIEA (45 mg, 345 μmol, 5.2 equiv) and HATU (33 mg, 86 μmol, 1.3 eq). The mixture was stirred at 20° C. for 0.5 h and concentrated. The residue was purified by prep-HPLC (column: Phenomenex C18 75*30 mm*3 m; mobile phase: [water(FA)-ACN]; B%: 8%-38%, 7 min) to give 4-[[(7R)-8-cyclopentyl-7-ethyl-5-methyl-6-oxo-7H-pteridin-2-yl]amino]-N-[2-[2-1[2-[2-[2-[[(E)-4-[2-1[2-(dimethylamino)ethyl-methyl-amino]-4-methoxy-5-[1[4-(1-methylindol-3-yl)pyrimidin-2-yl]amino]anilino]-4-oxo-but-2-enyl]-methyl-amino]ethoxy]ethoxy]ethoxy]ethoxy]ethyl]-3-methoxy-benzamide (14 mg, 17% yield). ¹H NMR (400 MHz, MeOD) δ8.81 (s, 1H), 8.41-8.34 (m, 1H), 8.32-8.25 (m, 1H), 8.23-8.14 (m, 2H), 7.69 (s, 1H), 7.46-7.36 (m, 3H), 7.28-7.10 (m, 3H), 7.01-6.81 (m, 3H), 4.42-4.32 (m, 1H), 4.27-4.19 (m, 1H), 4.03-3.78 (m, 13H), 3.71-3.43 (m, 20H), 3.28 (s, 3H), 3.25-3.19 (m, 2H), 2.92-2.78 (m, 9H), 2.72 (s, 3H), 2.19-2.07 (m, 1H), 1.88-1.57 (m, 9H), 0.86-0.75 (m, 3H). LC-MS: MS (ES⁺): RT=1.401 min, m/z=1169.6 [M+H⁺]; LCMS Method: 25.

### Example 108—Synthesis of Compound I-124

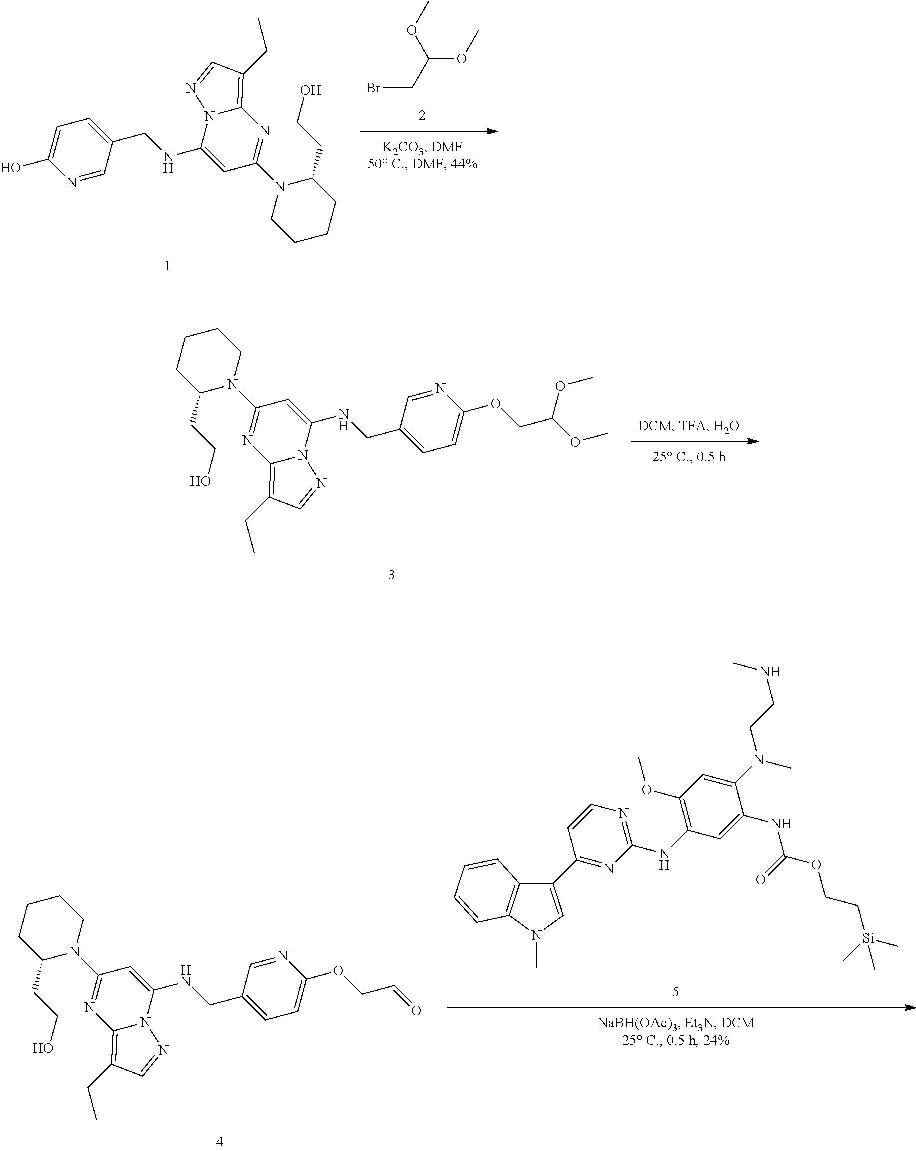

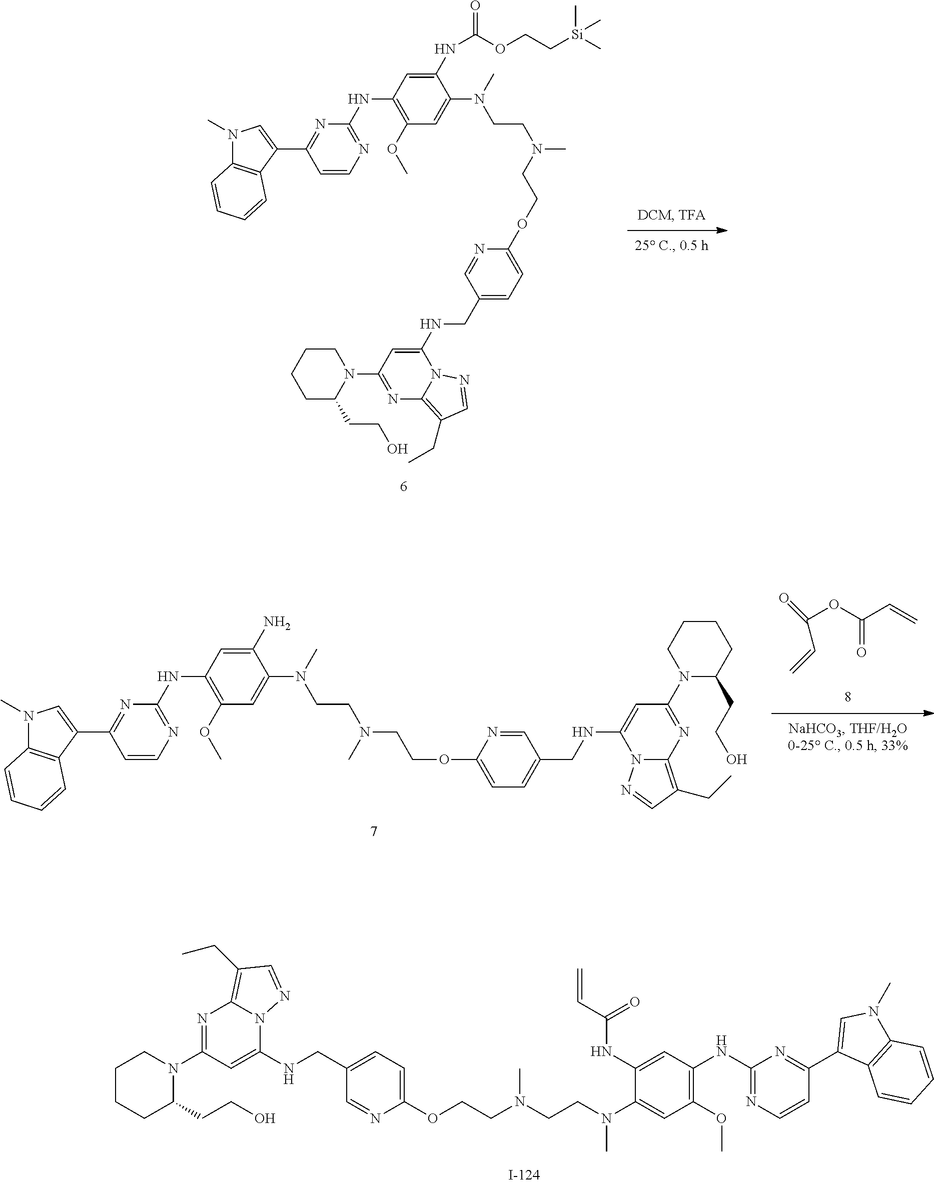

Step 1. To a solution of 2-bromo-1,1-dimethoxy-ethane (70.3 mg, 416.6 μmol, 48.8 L, 1.1 equiv) and 5-[[[3-ethyl-5-[(2S)-2-(2-hydroxyethyl)-1-piperidyl]pyrazolo[1,5-a]pyrimidin-7-yl]amino]methyl]pyridin-2-ol (150 mg, 378.3 μmol, 1 equiv) in DMF (2 mL) was added K₂CO₃ (156.8 mg, 1.3 mmol, 3 equiv). The mixture was stirred at 50° C. for 12 h. The residue was purified by prep-HPLC (column: Waters xbridge 150*25 mm*10 μm; mobile phase: [water (NH₄HCO₃)-ACN]; B%: 35%-65%, 10 min) to give compound 2-[(2S)-1-[7-[[6-(2,2-dimethoxyethoxy)-3-pyridyl]methylamino]-3-ethyl-pyrazolo [1,5-a]pyrimidin-5-yl]-2-piperidyl]ethanol (80 mg, 44% yield) as a colorless oil.

Step 2. A solution of 2-[(2S)-1-[7-[[6-(2,2-dimethoxyethoxy)-3-pyridyl]methylamino]-3-ethyl-pyrazolo[1,5-a]pyrimidin-5-yl]-2-piperidyl]ethanol (80 mg, 165.9 μmol, 1.0 equiv) in DCM/TFA/H₂O (1 mL, 10/1/0.2) was stirred at 25° C. for 0.5 h. Compound 2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]acetaldehyde (72 mg, 99% yield) was obtained as a colorless liquid and used for the next step directly.

Step 3. To a solution of 2-trimethylsilylethyl N-[4-methoxy-5-[[4-(1-methylindol-3-yl)pyrimidin-2-yl]amino]-2-[methyl-[2-(methylamino)ethyl]amino]phenyl]carbamate (94.5 mg, 164.9 μmol, 1.0 equiv) in DCM (2 mL) was added Et₃N (72.7 mg, 718.6 μmol, 0.1 mL, 4.8 equiv) and NaBH(OAc)₃ (347.9 mg, 1.6 mmol, 10.0 equiv), and then 2-[[5-[[[3-ethyl-5-[(2S)-2-(2-hydroxyethyl)-1-piperidyl]pyrazolo[ 1,5-a]pyrimidin-7-yl]amino]methyl]-2-pyridyl]oxy]acetaldehyde (72 mg, 164.9 μmol, 1.0 equiv) was added. The mixture was stirred at 25° C. for 0.5 h. The reaction mixture was concentrated to afford crude product. The residue was purified by prep-HPLC (column: Phenomenex C18 75*30 mm*3 m; mobile phase: [water (FA)-ACN]; B%: 22%-52%, 7 min) to give compound 2-trimethylsilylethyl N-[2-[2-[2-1[5-[[[3-ethyl-5-[(2S)-2-(2-hydroxyethyl)-1-piperidyl]pyrazolo[ 1,5-a]pyrimidin-7-yl]amino]methyl]-2-oxo-1-pyridyl]ethyl-methyl-amino]ethyl-methyl-amino]-4-methoxy-5-[[4-(1-methylindol-3-yl)pyrimidin-2-yl]amino]phenyl]carbamate (40 mg, 24% yield) as a colorless oil.

Step 4. To a solution of 2-trimethylsilylethyl N-[2-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethyl-methyl-amino]ethyl-methyl-amino]-4-methoxy-5-[[4-(1-methylindol-3-yl)pyrimidin-2-yl]amino]phenyl]carbamate (20.0 mg, 20.03 μmol, 1.0 equiv) in DCM (0.5 mL) was added TFA (0.2 mL), and then it was stirred at 25° C. for 0.5 h. 1-[2-[2-[2-amino-5-methoxy-N-methyl-4-[[4-(1-methylindol-3-yl)pyrimidin-2-yl]amino]anilino]ethyl-methyl-amino]ethyl]-5-1[[[3-ethyl-5-[(2S)-2-(2-hydroxyethyl)-1-piperidyl]pyrazolo[ 1,5-a]pyrimidin-7-yl]amino]methyl]pyridin-2-one (17 mg, 99% yield) as a colorless liquid and it was used for the next step directly.

Step 5. To a solution of 1-[2-[2-[2-amino-5-methoxy-N-methyl-4-[[4-(1-methylindol-3-yl)pyrimidin-2-yl]amino]anilino]ethyl-methyl-amino]ethyl]-5-[[[3-ethyl-5-[(2S)-2-(2-hydroxyethyl)-1-piperidyl]pyrazolo[1,5-a]pyrimidin-7-yl]amino]methyl]pyridin-2-one (17 mg, 19.9 μmol, 1.0 equiv), NaHCO₃ (90 mg, 0.64 mmol) in THF (1 mL) and H₂O (0.5 mL) was added prop-2-enoyl prop-2-enoate (3.1 mg, 23.9 μmol, 1.2 equiv) at 0° C., and then it was stirred at 25° C. for 0.5 h. The reaction mixture was concentrated to afford crude product. The residue was purified by prep-HPLC (column: Waters Xbridge 150*25 mm*5 m; mobile phase: [water (NH₄HCO₃)-ACN]; B%: 48%-78%, 10 min) to give compound N-[2-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethyl-methyl-amino]ethyl-methyl-amino]-4-methoxy-5-[[4-(1-methylindol-3-yl)pyrimidin-2-yl]amino]phenyl]prop-2-enamide (5.99 mg, 33% yield, 100% purity) as a white solid. ¹H NMR (400 MHz, CD₃OD): δ 9.51 (s, 1H), 8.64-8.73 (m, 1H), 8.28 (d, J=5.50 Hz, 1H), 8.11-8.20 (m, 1H), 7.64 (s, 1H), 7.57 (s, 1H), 7.46 (s, 2H), 7.18-7.28 (m, 3H), 6.92 (s, 1H), 6.51 (d, J=9.26 Hz, 1H), 6.30-6.40 (m, 2H), 5.73 (m, 1H), 5.28 (s, 1H), 4.14 (s, 2H), 4.06-4.09 (m, 3H), 3.91 (d, J=4.63 Hz, 7H), 3.45-3.49 (m, 1H), 2.91-2.99 (m, 2H), 2.71-2.88 (m, 7H), 2.49-2.61 (m, 4H), 2.35 (s, 3H), 1.88-2.02 (m, 1H), 1.47-1.71 (m, 7H), 1.33-1.44 (m, 1H), 1.16-1.22 (m, 3H). LC-MS: MS (ES′): RT=1.54 min, m/z=908.4 [M+H⁺]; LCMS method: 25.

### Example 109—Synthesis of Compound I-125

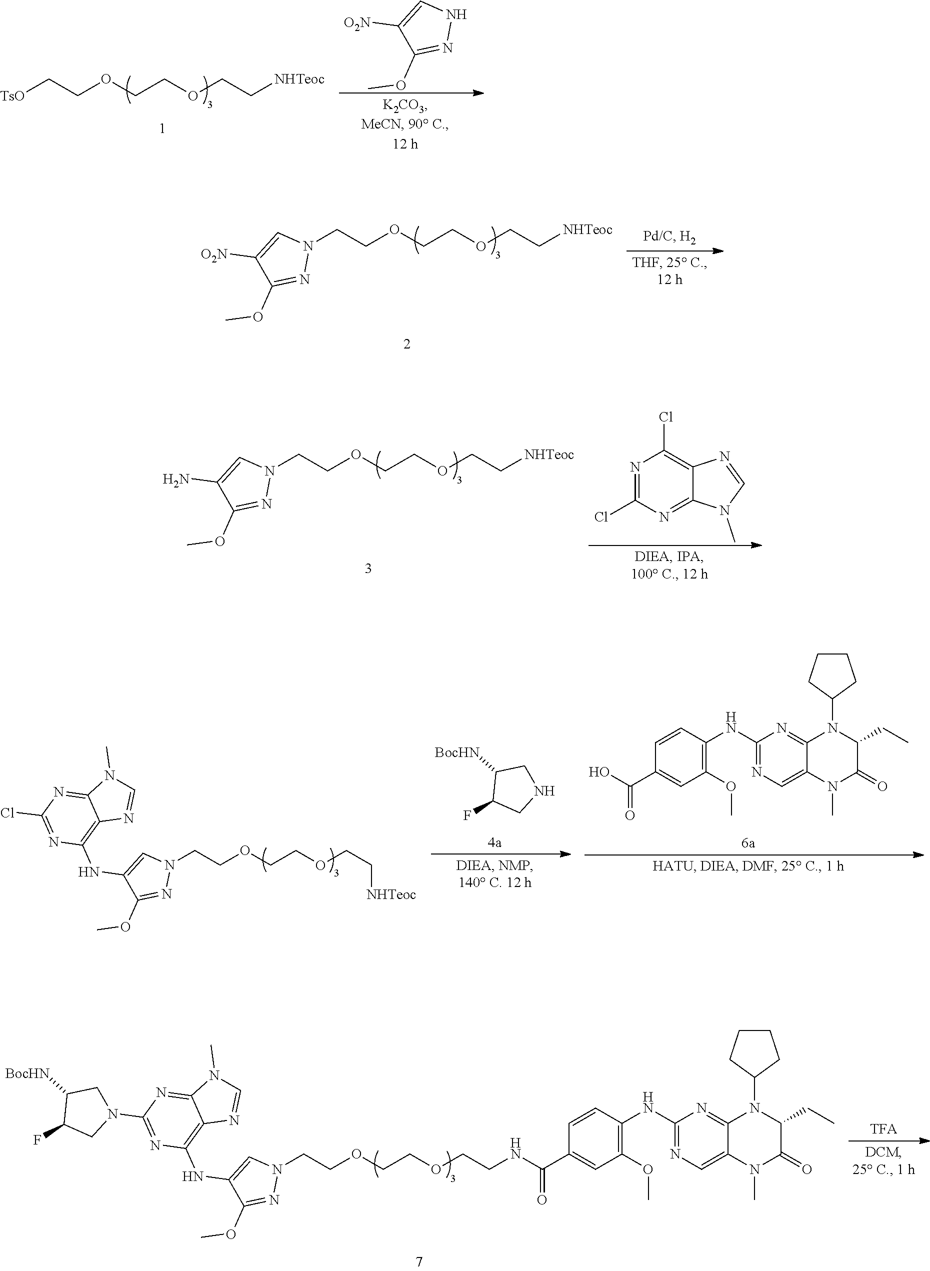

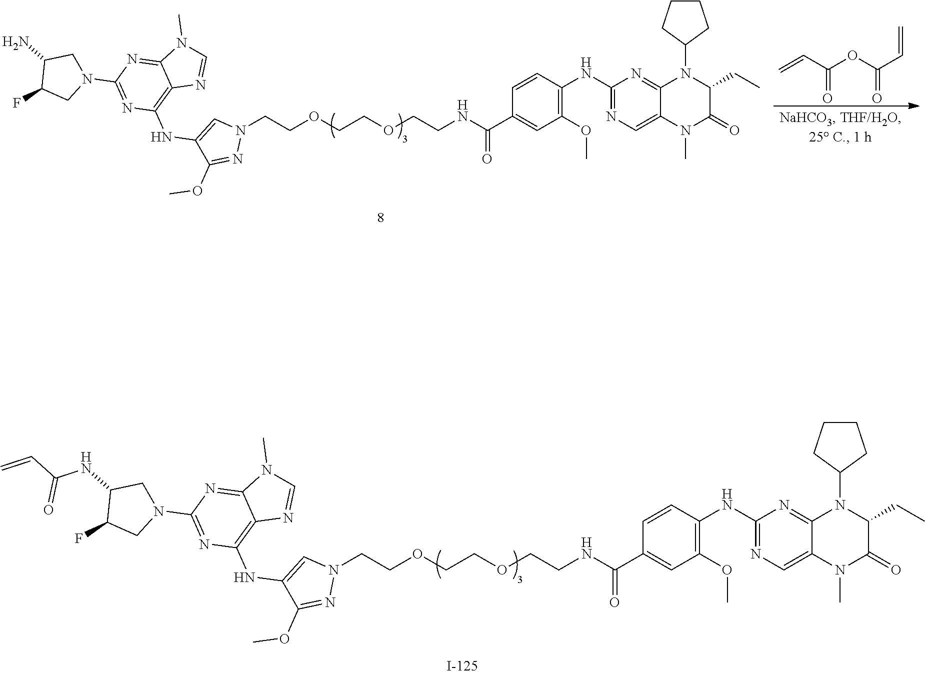

General Information: Compound 6a is a known compound from WO2017/24317.

Step 1. To a solution of 3-methoxy-4-nitro-1H-pyrazole (187 mg, 1.31 mmol, 1.0 equiv) and 2-[2-[2-[2-[2-(2-trimethylsilylethoxycarbonylamino)ethoxy]ethoxy]ethoxy]ethoxy]ethyl 4-methylbenzenesulfonate (700 mg, 1.31 mmol, 1.0 equiv) in MeCN (2 mL) was added K₂CO₃ (542 mg, 3.93 mmol, 3.0 equiv). The mixture was stirred at 90° C. for 12 h. The mixture was filtered and concentrated to give a residue. The residue was purified by prep-HPLC (column: Phenomenex luna C18 150*40 mm*15 m; mobile phase: [water(FA)-ACN]; B%: 42%-72%, 10 min) to give 2-trimethylsilylethyl N-[2-[2-[2-[2-[2-(3-methoxy-4-nitro-pyrazol-1-yl)ethoxy]ethoxy]ethoxy]ethoxy]ethyl]carbamate (430 mg, 65% yield) as a yellow oil.

Step 2. To a solution of 2-trimethylsilylethyl N-[2-[2-[2-[2-[2-(3-methoxy-4-nitro-pyrazol-1-yl)ethoxy]ethoxy]ethoxy]ethoxy]ethyl]carbamate (430 mg, 4.3 mmol, 1.0 equiv) in THF (10 mL) was added Pd/C (100 mg, 10% purity) under N2. The suspension was degassed under vacuum and purged with H₂ for three times. The mixture was stirred under H₂ (15 psi) at 25° C. for 12 h. The mixture was filtered and concentrated under reduced pressure to give 2-trimethylsilylethyl N-[2-[2-[2-[2-[2-(4-amino-3-methoxy-pyrazol-1-yl)ethoxy]ethoxy]ethoxy]ethoxy]ethyl]carbamate (400 mg, 98% yield) as a yellow oil.

Step 3. To a solution of 2,6-dichloro-9-methyl-purine (150 mg, 734 μmol, 1.0 equiv) and 2-trimethylsilylethyl N—[2-[2-[2-[2-[22-(4-amino-3-methoxy-pyrazol-1-yl)ethoxy]ethoxy]ethoxy]ethoxy]ethyl]carbamate (350 mg, 734 μmol, 1.0 equiv) in IPA (6 mL) was added DIEA (380 mg, 2.94 mmol, 4.0 equiv). The mixture was stirred at 100° C. for 12 h. The mixture was purified by prep-HPLC (column: Waters Xbridge C18 150*50 mm*10 m; mobile phase: [water(NH4HCO₃)-ACN]; B%: 36%-66%, 11 min) to give 2-trimethylsilylethyl N-[2-[2-[2-[2-[2-[4-[(2-chloro-9-methyl-purin-6-yl)amino]-3-methoxy-pyrazol-1-yl]ethoxy]ethoxy]ethoxy]ethoxy]ethyl]carbamate (470 g, 99% yield) as a yellow oil.

Step 4. To a solution of 2-trimethylsilylethyl N-[2-[2-[2-[2-[2-[4-[(2-chloro-9-methyl-purin-6-yl)amino]-3-methoxy-pyrazol-1-yl]ethoxy]ethoxy]ethoxy]ethoxy]ethyl]carbamate (380 mg, 590 μmol, 1.0 equiv) and tert-butyl N-[(3R,4R)-4-fluoropyrrolidin-3-yl]carbamate (169 mg, 827 μmol, 1.4 equiv) in NMP (5 mL) was added DIEA (382 mg, 2.95 mmol, 5.0 equiv). The mixture was stirred at 140° C. for 12 h. The mixture was purified by prep-HPLC (column: Waters Xbridge C18 150*50 mm*10 m; mobile phase: [water(NH₄HCO₃)-ACN]; B%: 44%-74%, 11 min) to give 2-trimethylsilylethyl N-[2-[2-[2-[2-[2-[4-[[2-[(3R,4R)-3-(tert-butoxycarbonylamino)-4-fluoro-pyrrolidin-1-yl]-9-methyl-purin-6-yl]amino]-3-methoxy-pyrazol-1-yl]ethoxy]ethoxy]ethoxy]ethoxy]ethyl]carbamate (210 mg, 44% yield) as a yellow oil.

Step 5. To a solution of 2-trimethylsilylethyl N-[2-[2-[2-[2-[2-[4-[[2-[(3R,4R)-3-(tert-butoxycarbonylamino)-4-fluoro-pyrrolidin-1-yl]-9-methyl-purin-6-yl]amino]-3-methoxy-pyrazol-1-yl]ethoxy]ethoxy]ethoxy]ethoxy]ethyl]carbamate (210 mg, 259 μmol, 1.0 equiv) in THF (1 mL) was added TBAF (1 M, 0.52 mL, 2.0 equiv). The mixture was stirred at 25° C. for 12 h. The reaction mixture was concentrated under reduced pressure to give a residue. The residue was purified by prep-HPLC (column: Waters Xbridge 150*25 mm*5 m; mobile phase: [water(NH₄HCO₃)-ACN]; B%: 31%-61%, 10 min) to afford tert-butyl N-[(3R,4R)-1-[6-[[1-[2-[2-[2-1[2-(2-aminoethoxy)ethoxy]ethoxy]ethoxy]ethyl]-3-methoxy-pyrazol-4-yl]amino]-9-methyl-purin-2-yl]-4-fluoro-pyrrolidin-3-yl]carbamate (50 mg, 29% yield) as a colorless oil.

Step 6. To a solution of tert-butyl N-[(3R,4R)-1-[6-[[1-[2-[2-[2-[2-(2-aminoethoxy)ethoxy]ethoxy]ethoxy]ethyl]-3-methoxy-pyrazol-4-yl]amino]-9-methyl-purin-2-yl]-4-fluoro-pyrrolidin-3-yl]carbamate (40 mg, 60 μmol, 1.0 equiv) and 4-[[(7R)-8-cyclopentyl-7-ethyl-5-methyl-6-oxo-7H-pteridin-2-yl]amino]-3-methoxy-benzoic acid (26 mg, 60 μmol, 1.0 equiv) in DMF (2 mL) was added DIEA (39 mg, 300 μmol, 5.0 equiv) and HATU (27 mg, 72 μmol, 1.2 equiv) and. The mixture was stirred at 25° C. for 1 h. The residue was purified by prep-HPLC (column: Unisil 3-100 C18 Ultra 150*50 mm*3 m; mobile phase: [water(FA)-ACN]; B%: 23%-53%, 10 min) to give tert-butyl N-[(3R,4R)-1-[6-[[1-[2-[2-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethyl]-3-methoxy-pyrazol-4-yl]amino]-9-methyl-purin-2-yl]-4-fluoro-pyrrolidin-3-yl]carbamate (55 mg, 85% yield) as a brown solid.

Step 7. To a solution of tert-butyl N-[(3R,4R)-1-[6-[[1-[2-[2-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethyl]-3-methoxy-pyrazol-4-yl]amino]-9-methyl-purin-2-yl]-4-fluoro-pyrrolidin-3-yl]carbamate (45 mg, 42 μmol, 1.0 equiv) in DCM (1 mL) was added TFA (0.5 mL). The mixture was stirred at 25° C. for 1 h. The mixture was concentrated under reduced pressure to give N—[[2[2-[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy]ethoxy]ethoxy]ethyl]-4-[[(7R)-8-cyclopentyl-7-ethyl-5-methyl-6-oxo-7H-pteridin-2-yl]amino]-3-methoxy-benzamide (40 mg, crude) as a colorless oil without purification for the next step.

Step 8. To a solution of N-[2-[2-[2-[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy]ethoxy]ethoxy]ethyl]-4-[[(7R)-8-cyclopentyl-7-ethyl-5-methyl-6-oxo-7H-pteridin-2-yl]amino]-3-methoxy-benzamide (40 mg, 41 μmol, 1.0 equiv) in THF (2 mL) and NaHCO₃ (1 M, 1 mL, 24.4 equiv) in H₂O (1 mL) was added prop-2-enoyl prop-2-enoate (6.7 mg, 54 μmol, 1.3 equiv) at 0° C. The mixture was stirred at 25° C. for 1 h. The mixture was diluted with water (20 mL) and extracted with EtOAc (3×10 mL). The combined organic layers were washed with brine (20 mL), dried over sodium sulfate, filtered and concentrated. The residue was purified by prep-HPLC (column: Unisil 3-100 C18 Ultra 150*50 mm*3 m; mobile phase: [water(FA)-ACN]; B%: 17%-47%, 10 min) to give 4-[[(7R)-8-cyclopentyl-7-ethyl-5-methyl-6-oxo-7H-pteridin-2-yl]amino]-N-[2-2-[2-[2-[2-[14-[[2-[(3R,4R)-3-fluoro-4-(prop-2-enoylamino)pyrrolidin-1-yl]-9-methyl-purin-6-yl]amino]-3-methoxy-pyrazol-1-yl]ethoxy]ethoxy]ethoxy]ethoxy]ethyl]-3-methoxy-benzamide (9.7 mg, 22.% yield) as a white solid. ¹H NMR (400 MHz, DMSO-d₆): δ8.40-8.30 (m, 1H), 8.02-7.91 (m, 1H), 7.61-7.53 (m, 2H), 7.46-7.39 (m, 2H), 7.30 (d, J=9.0 Hz, 2H), 7.19-7.12 (m, 1H), 6.43-6.36 (m, 1H), 6.33-6.23 (m, 1H), 5.64 (d, J=9.8, 1H), 5.31 (s, 1H), 4.72 (d, J=3.6 Hz, 1H), 4.51-4.39 (m, 1H), 4.24-4.15 (m, 1H), 3.94 (d, J=16.0 Hz, 10H), 3.77-3.71 (m, 2H), 3.64 (s, 3H), 3.62-3.48 (m, 18H), 3.30 (s, 3H), 2.21-2.11 (m, 1H), 2.05-1.98 (m, 1H), 1.73-1.63 (m, 6H), 1.29-1.23 (m, 3H), 0.84 (t, J=7.4 Hz, 3H). LC-MS: MS (ES′): RT=2.211 min, m/z=1028.4 [M+H⁺]; LCMS method: 10.

### Example 110—Synthesis of Compound I-126

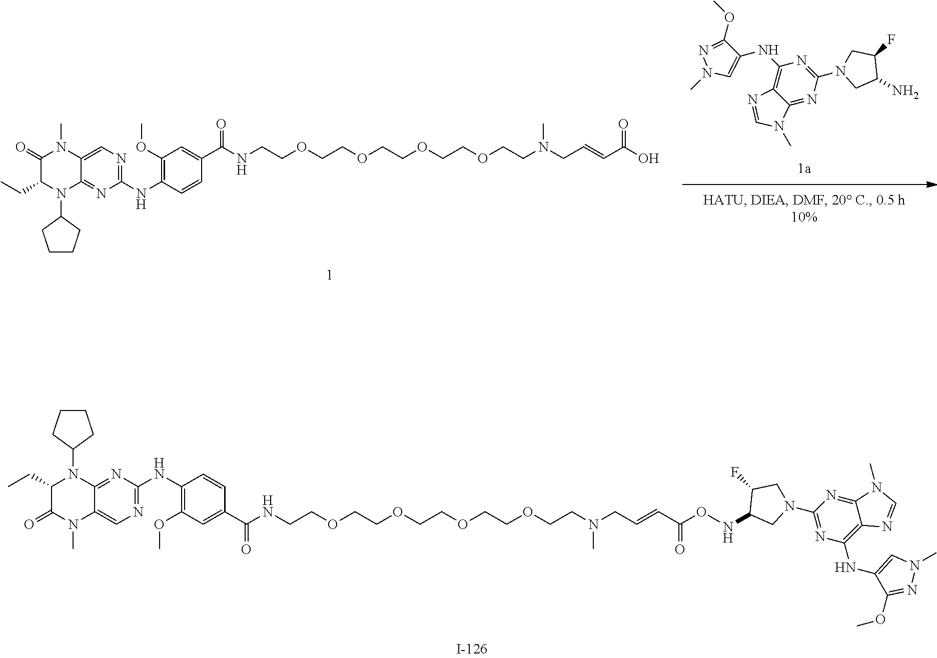

Step 1. To a solution of (E)-4-[2-[2-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethyl-methyl-amino]but-2-enoic acid (35 mg, 47 μmol, 1.0 equiv) in DMF (1 mL) was added 2-[(3R,4R)-3-amino-4-fluoro-pyrrolidin-1-yl]-N-(3-methoxy-1-methyl-pyrazol-4-yl)-9-methyl-purin-6-amine (19 mg, 47 μmol, 1.0 equiv, HCl) and DIEA (31 mg, 236 μmol, 5.0 equiv) and HATU (23 mg, 61 μmol, 1.3 equiv).The mixture was stirred at 20° C. for 0.5 h and concentrated. The residue was purified by prep-HPLC (column: Phenomenex C18 75*30 mm*3 m; mobile phase: [water(FA)-ACN]; B%: 12%-42%, 7 min) to give 4-[[(7R)-8-cyclopentyl-7-ethyl-5-methyl-6-oxo-7H-pteridin-2-yl]amino]-N-[2-[2-[2-[2-[2-[[(E)-4-[ [(3R,4R)-4-fluoro-1-1[6-1[(3-methoxy-1-methyl-pyrazol-4-yl)amino]-9-methyl-purin-2-yl]pyrrolidin-3-yl]amino]-4-oxo-but-2-enyl]-methyl-amino]ethoxy]ethoxy]ethoxy]ethoxy]ethyl]-3-methoxy-benzamide (5 mg, 10% yield). ¹H NMR (400 MHz, MeOD) δ8.43-8.39 (m, 1H), 7.94 (s, 1H), 7.76-7.69 (m, 2H), 7.48-7.42 (m, 2H), 6.90-6.82 (m, 1H), 6.26-6.20 (m, 1H), 5.29-5.13 (m, 1H), 4.76-4.58 (m, 3H), 4.51-4.41 (m, 1H), 4.31-4.26 (m, 1H), 4.04-3.86 (m, 9H), 3.78-3.43 (m, 24H), 3.31 (s, 4H), 2.68-2.58 (m, 2H), 2.34 (s, 3H), 2.23-2.15 (m, 1H), 2.10-1.99 (m, 1H), 1.97-1.84 (m, 4H), 1.80-1.68 (m, 4H), 0.88-0.81 (m, 3H). LC-MS: MS (ES′): RT=2.040 min, m/z=1085.5 [M+H⁺]; LCMS Method: 10.

### Example 111—Synthesis of Compound I-127

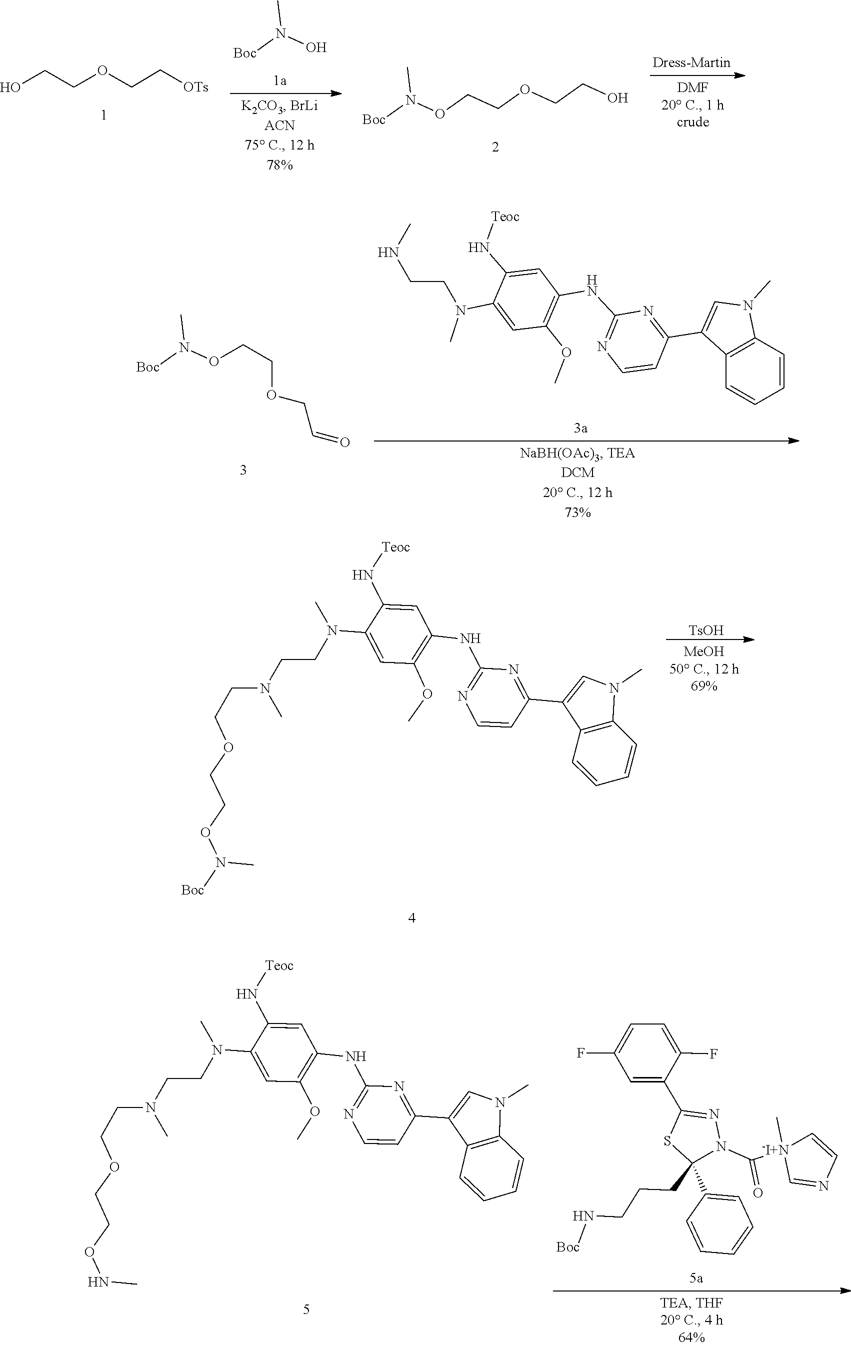

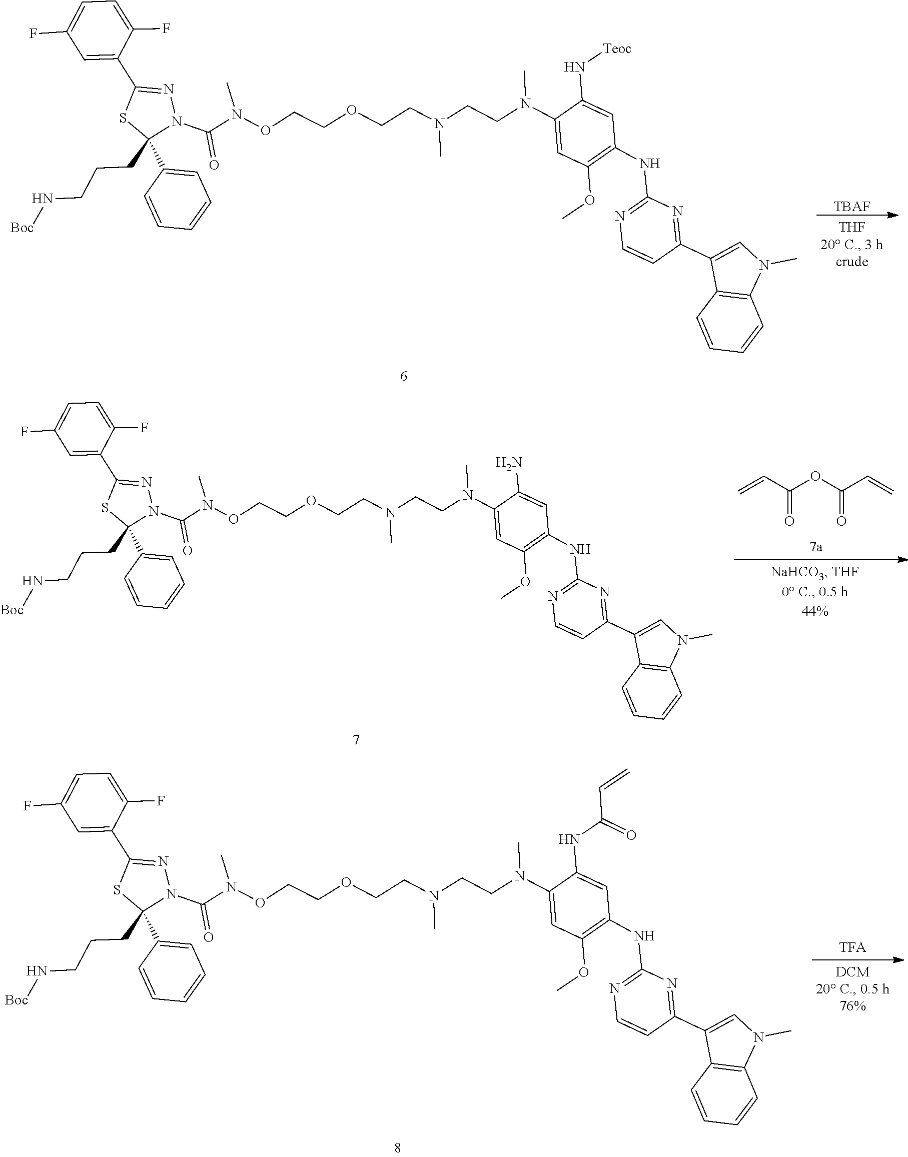

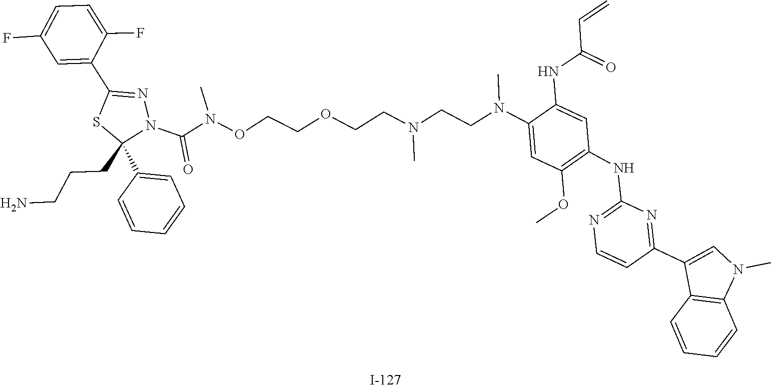 [SMILES: C=CC(=O)Nc1cc(Nc2nccc(-c3cn(C)c4ccccc34)n2)c(OC)cc1N(C)CCN(C)CCOCCON(C)C(=O)N1N=C(c2cc(F)ccc2F)S[C@@]1(CCCN)c1ccccc1]

General Information: Compound 5a is a known compound from WO2006/44825, 2006, A2.

Step 1. To a solution of 2-(2-hydroxyethoxy)ethyl 4-methylbenzenesulfonate (1 g, 4 mmol, 1.0 equiv) and tert-butyl N-hydroxy-N-methyl-carbamate (565 mg, 4 mmol, 1.0 equiv) in ACN (10 mL) was added K₂CO₃ (1 g, 8 mmol, 2.0 equiv) and LiBr (7 mg, 77 μmol, 0.02 equiv). The mixture was stirred at 75° C. for 12 h and concentrated. The residue was purified by column chromatography (SiO₂, Petroleum ether/Ethyl acetate=20/1 to 3/1) to give tert-butyl N-[2-(2-hydroxyethoxy)ethoxy]-N-methyl-carbamate (704 mg, 78% yield). ¹H NMR (400 MHz, CDCl₃): δ 4.01-4.03 (m, 2H), 3.69-3.71 (m, 2H), 3.11 (s, 3H), 1.49 (s, 9H).

Step 2. To a solution of tert-butyl N-[2-(2-hydroxyethoxy)ethoxy]-N-methyl-carbamate (150 mg, 638 μmol, 1.0 equiv) in DMF (2 mL) was added Dess-Martin (676 mg, 1.6 mmol, 2.5 equiv). The mixture was stirred at 20° C. for 1 h. The solution was used for next step directly.

Step 3. To a solution of tert-butyl N-methyl-N-[2-(2-oxoethoxy)ethoxy]carbamate (148 mg, 634 μmol, 1.0 equiv) in DCM (5 mL) was added TEA (449 mg, 4.4 mmol, 7.0 equiv) and NaBH(OAc)₃ (1.4 g, 6.4 mmol, 10.0 equiv), and then 2-trimethylsilylethyl N-[4-methoxy-5-[ [4-(1-methylindol-3-yl)pyrimidin-2-yl]amino]-2-[methyl-[2-(methylamino)ethyl]amino]phenyl]carbamate (365.32 mg, 635 μmol, 1.0 equiv) was added. The mixture was stirred at 20° C. for 12 h and concentrated. The residue was purified by column chromatography (SiO₂, Petroleum ether/Ethyl acetate=20/1 to 0/1) to give tert-butyl N-[2-[2-[2-[5-methoxy-N-methyl-4-[[4-(1-methylindol-3-yl)pyrimidin-2-yl]amino]-2-(2-trimethylsilylethoxycarbonylamino)anilino]ethyl-methyl-amino]ethoxy]ethoxy]-N-methyl-carbamate (365 mg, 73% yield) as a brown oil. ¹H NMR (400 MHz, CDCl₃): δ 9.43 (s, 1H), 8.39 (d, J=5.4 Hz, 1H), 8.07-8.16 (m, 1H), 7.93 (d, J=7.4 Hz, 1H), 7.75-7.85 (m, 2H), 7.20 (d, J=5.4 Hz, 1H), 7.04-7.08 (m, 1H), 6.80 (s, 1H), 4.22-4.28 (m, 2H), 3.87 (s, 3H), 3.79 (t, J=5.2 Hz, 2H), 3.61-3.64 (m, 2H), 3.50 (s, 6H), 3.20 (t, J=6.8 Hz, 2H), 3.07 (s, 3H), 3.03 (t, J=5.4 Hz, 2H), 2.86 (t, J=6.8 Hz, 3H), 2.68 (s, 3H), 2.62 (s, 3H), 1.47 (s, 9H), 1.01-1.07 (m, 2H), 0.06 (s, 9H).

Step 4. To a solution of tert-butyl N-[2-[2-[2-[5-methoxy-N-methyl-4-[[4-(1-methylindol-3-yl)pyrimidin-2-yl]amino]-2-(2-trimethylsilylethoxycarbonylamino)anilino]ethyl-methyl-amino]ethoxy]ethoxy]-N-methyl-carbamate (365 mg, 460 μmol, 1.0 equiv) in MeOH (3.8 mL) was added TosOH (198 mg, 1.2 mmol, 2.5 equiv).The mixture was stirred at 50° C. for 12 h and concentrated. The residue was purified by prep-HPLC (column: Waters Xbridge C18 150*50 mm*10 m; mobile phase:[water(NH₄HCO₃)-ACN]; B%: 68%-98%, 11 min) to give 2-trimethylsilylethylN-[4-methoxy-5-[[4-(1-methylindol-3-yl)pyrimidin-2-yl]amino]-2-[methyl-[2-[methyl-[2-[2-(methylaminooxy)ethoxy]ethyl]amino]ethyl]amino]phenyl]carbamate (219 mg, 69% yield). ¹H NMR (400 MHz, CDCl₃): δ 9.44 (s, 1H), 8.73-8.84 (m, 1H), 8.39 (d, J=5.4 Hz, 1H), 8.13 (d, J=6.8 Hz, 1H), 7.73-7.83 (m, 2H), 7.38-7.43 (m, 1H), 7.17-7.23 (m, 2H), 6.79 (s, 1H), 4.24-4.30 (m, 2H), 3.96 (s, 3H), 3.87 (s, 3H), 3.77-3.80 (m, 2H), 3.66 (d, J=2.2 Hz, 1H), 3.58-3.61 (m, 2H), 3.17 (d, J=6.8 Hz, 2H), 2.96-3.04 (m, 2H), 2.80-2.88 (m, 2H), 2.65-2.68 (m, 6H), 2.62 (s, 3H), 2.36 (s, 1H), 1.27 (t, J=7.2 Hz, 2H), 1.04-1.11 (m, 2H), 0.05-0.09 (m, 9H).

Step 5. To a solution of 2-trimethylsilylethyl N-[4-methoxy-5-[[4-(1-methylindol-3-yl)pyrimidin-2-yl]amino]-2-[methyl-[2-[methyl-[2-[2-(methylaminooxy)ethoxy]ethyl]amino]ethyl]amino]phenyl]carbamate (119 mg, 172 μmol, 1.0 equiv) and tert-butyl N-[3-[(2S)-5-(2,5-difluorophenyl)-3-(1-methylimidazol-1-ium-1-carbonyl)-2-phenyl-1,3,4-thiadiazol-2-yl]propyl]carbamate; iodide (173 mg, 258 μmol, 1.5 equiv) in THF (3 mL) was added TEA (56 mg, 555 μmol, 3.2 equiv). The mixture was stirred at 20° C. for 4 h and concentrated. The residue was purified by prep-TLC (SiO₂, EtOAc: MeOH=10:1) to give 2-trimethylsilylethyl N-[2-[2-[2-[2-[[(2S)-2-[3-(tert-butoxycarbonylamino)propyl]-5-(2,5-difluorophenyl)-2-phenyl-1,3,4-thiadiazole-3-carbonyl]-methyl-amino]oxyethoxy]ethyl-methyl-amino]ethyl-methyl-amino]-4-methoxy-5-[[4-(1-methylindol-3-yl)pyrimidin-2-yl]amino]phenyl]carbamate (127 mg, 64% yield). ¹H NMR (400 MHz, CDCl₃): δ8.74-8.87 (m, 1H), 8.39 (d, J=5.2 Hz, 1H), 8.12 (d, J=6.6 Hz, 1H), 7.73 (s, 1H), 7.46 (d, J=7.2 Hz, 2H), 7.39-7.41 (m, 1H), 7.28-7.37 (m, 5H), 7.19 (d, J=5.2 Hz, 1H), 6.83-7.01 (m, 3H), 6.79 (s, 1H), 4.25-4.33 (m, 2H), 3.96 (s, 3H), 3.89 (s, 3H), 3.57 (s, 3H), 3.15-3.31 (m, 7H), 3.04-3.14 (m, 2H), 2.65 (s, 3H), 2.35-2.43 (m, 2H), 1.90-2.03 (m, 2H), 1.57-1.65 (m, 2H), 1.43 (s, 11H), 1.05-1.13 (m, 4H), 0.85-0.88 (m, 2H), 0.07 (s, 9H).

Step 6. To a solution of 2-trimethylsilylethyl N-[2-[2-[2-[2-[[(2S)-2-[3-(tert-butoxycarbonylamino)propyl]-5-(2,5-difluorophenyl)-2-phenyl-1,3,4-thiadiazole-3-carbonyl]-methyl-amino]oxyethoxy]ethyl-methyl-amino]ethyl-methyl-amino]-4-methoxy-5-[[4-(1-methylindol-3-yl)pyrimidin-2-yl]amino]phenyl]carbamate (127 mg, 110 μmol, 1.0 equiv) in THF (0.5 mL) was added TBAF (1 M, 0.5 mL, 4.5 equiv). The mixture was stirred at 20° C. for 3 h. The solution was used for next step directly.

Step 7. To a solution of tert-butyl N-[3-[(2S)-3-[2-[2-[2-[2-amino-5-methoxy-N-methyl-4-[[4-(1-methylindol-3-yl)pyrimidin-2-yl]amino]anilino]ethyl-methyl-amino]ethoxy]ethoxy-methyl-carbamoyl]-5-(2,5-difluorophenyl)-2-phenyl-1,3,4-thiadiazol-2-yl]propyl]carbamate (111 mg, 110 μmol, 1.0 equiv) in THF (1 mL) and NaHCO₃ (1.0 mL, 26 mmol, 234 equiv) was added prop-2-enoyl prop-2-enoate (14 mg, 110 μmol, 1.0 equiv). The mixture was stirred at 0° C. for 0.5 h and concentrated. The residue was purified by prep-TLC (SiO₂, EtOAc: MeOH=10:1) to give tert-butylN-[3-[(2S)-5-(2,5-difluorophenyl)-3-[2-[2-[2-[5-methoxy-N-methyl-4-[[4-(1-methylindol-3-yl)pyrimidin-2-yl]amino]-2-(prop-2-enoylamino)anilino]ethyl-methyl-amino]ethoxy]ethoxy-methyl-carbamoyl]-2-phenyl-1,3,4-thiadiazol-2-yl]propyl]carbamate (52 mg, 44% yield). ¹H NMR (400 MHz, CDCl₃): δ 9.87 (s, 1H), 9.69-9.80 (m, 1H), 9.10 (s, 1H), 8.39 (d, J=5.2 Hz, 1H), 8.04-8.09 (m, 1H), 7.64-7.76 (m, 2H), 7.47 (d, J=7.6 Hz, 2H), 7.38-7.42 (m, 1H), 7.31-7.37 (m, 2H), 7.28-7.30 (m, 1H), 7.21 (d, J=5.4 Hz, 1H), 6.81-6.97 (m, 2H), 6.79 (s, 1H), 6.46 (d, J=5.8 Hz, 2H), 5.70 (t, J=6.0 Hz, 1H), 4.48-4.80 (m, 1H), 4.06-4.12 (m, 2H), 4.00 (s, 3H), 3.89 (s, 3H), 3.50-3.64 (m, 4H), 3.22-3.30 (m, 2H), 2.96-3.22 (m, 5H), 2.85-2.95 (m, 2H), 2.68 (s, 3H), 2.63 (t, J=5.2 Hz, 2H), 2.38-2.44 (m, 2H), 2.29 (s, 2H) 2.05 (s, 3H), 1.58-1.63 (m, 2H), 1.44 (s, 9H).

Step 8. To a solution of tert-butyl N-[3-[(2S)-5-(2,5-difluorophenyl)-3-[2-[2-[2-[5-methoxy-N-methyl-4-[[4-(1-methylindol-3-yl)pyrimidin-2-yl]amino]-2-(prop-2-enoylamino) anilino]ethyl-methyl-amino]ethoxy]ethoxy-methyl-carbamoyl]-2-phenyl-1,3,4-thiadiazol-2-yl]propyl]carbamate (60 mg, 57 μmol, 1.0 equiv) in DCM (5 mL) was added TFA (0.5 mL). The mixture was stirred at 20° C. for 0.5 h and concentrated. The residue was purified by pre-HPLC (column: Phenomenex C18 75*30 mm*3 m; mobile phase: [water(FA)-ACN]; B%: 15%-45%, 7 min) to give (2S)-2-(3-aminopropyl)-5-(2,5-difluorophenyl)-N-[2-[2-[2-[5-methoxy-N-methyl-4-[[4-(1-methylindol-3-yl)pyrimidin-2-yl]amino]-2-(prop-2-enoylamino)anilino]ethyl-methyl-amino]ethoxy]ethoxy]-N-methyl-2-phenyl-1,3,4-thiadiazole-3-carboxamide (41 mg, 76% yield). ¹H NMR (400 MHz, MeOD): 9.41 (s, 1H), 8.75 (s, 1H), 8.68 (s, 1H), 8.49-8.50 (m, 1 H), 8.43 (d, J=7.8 Hz, 1H), 7.89-7.95 (m, 1H), 7.66 (d, J=7.8 Hz, 3H), 7.56 (t, J=7.6 Hz, 2H), 7.38-7.52 (m, 4H), 7.16-7.28 (m, 3H), 6.77-6.86 (m, 1H), 6.60-6.67 (m, 1H), 6.00-6.04 (m, 1H), 4.21-4.29 (m, 1H), 4.17 (s, 3H), 4.12 (d, J=0.8 Hz, 2H), 3.73-3.80 (m, 2H), 3.69 (t, J=4.4 Hz, 2H), 3.17-3.47 (m, 9H), 3.04 (s, 4H), 2.91 (s, 3H), 2.56-2.80 (m, 4H), 2.18-2.40 (m, 1H), 1.87-1.98 (m, 1H). LC-MS: MS (ES′): RT=1.719 min, m/z=962.3 [M+H]+; LCMS method: 25.

### Example 112—Synthesis of Compound I-128

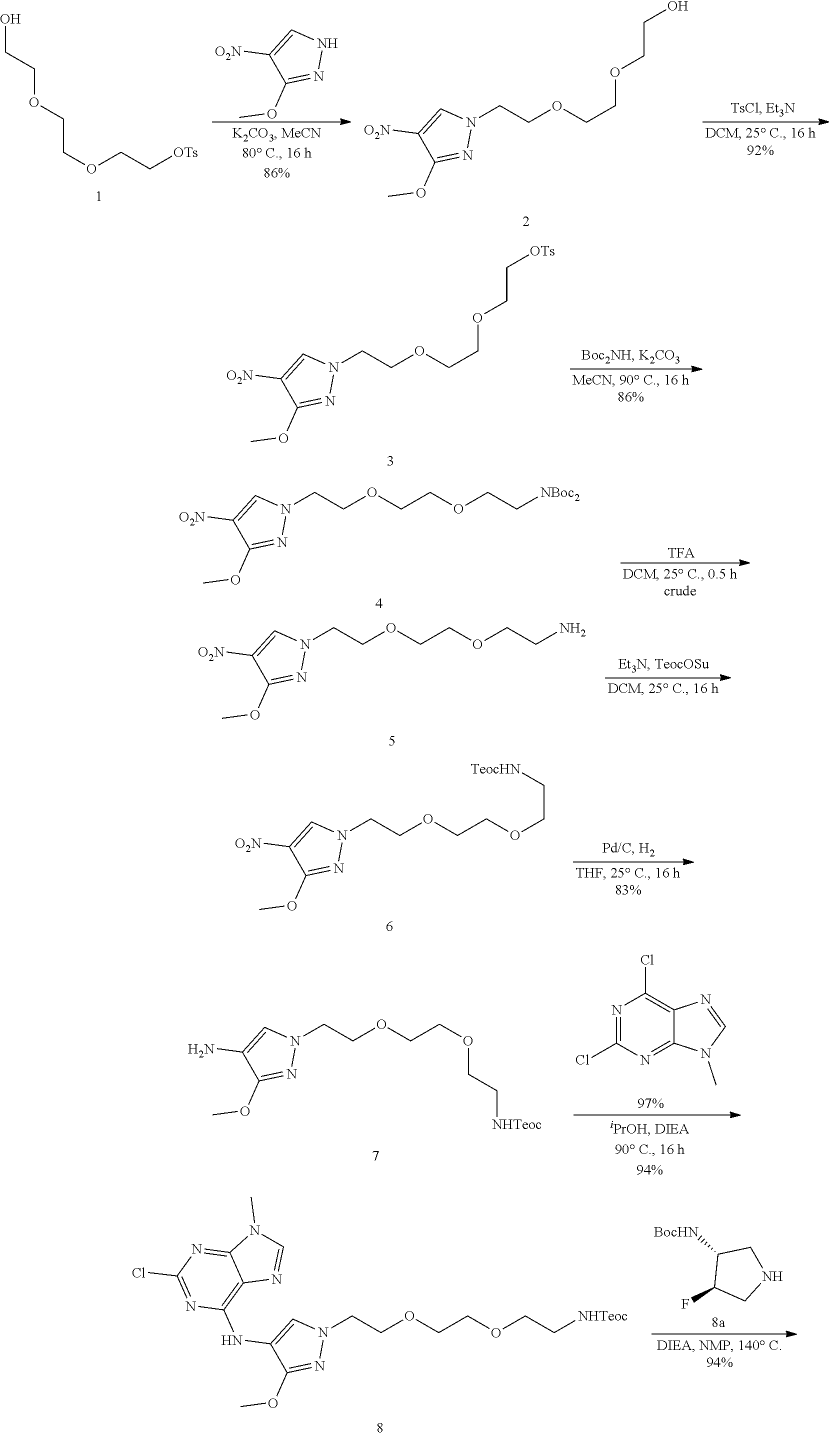

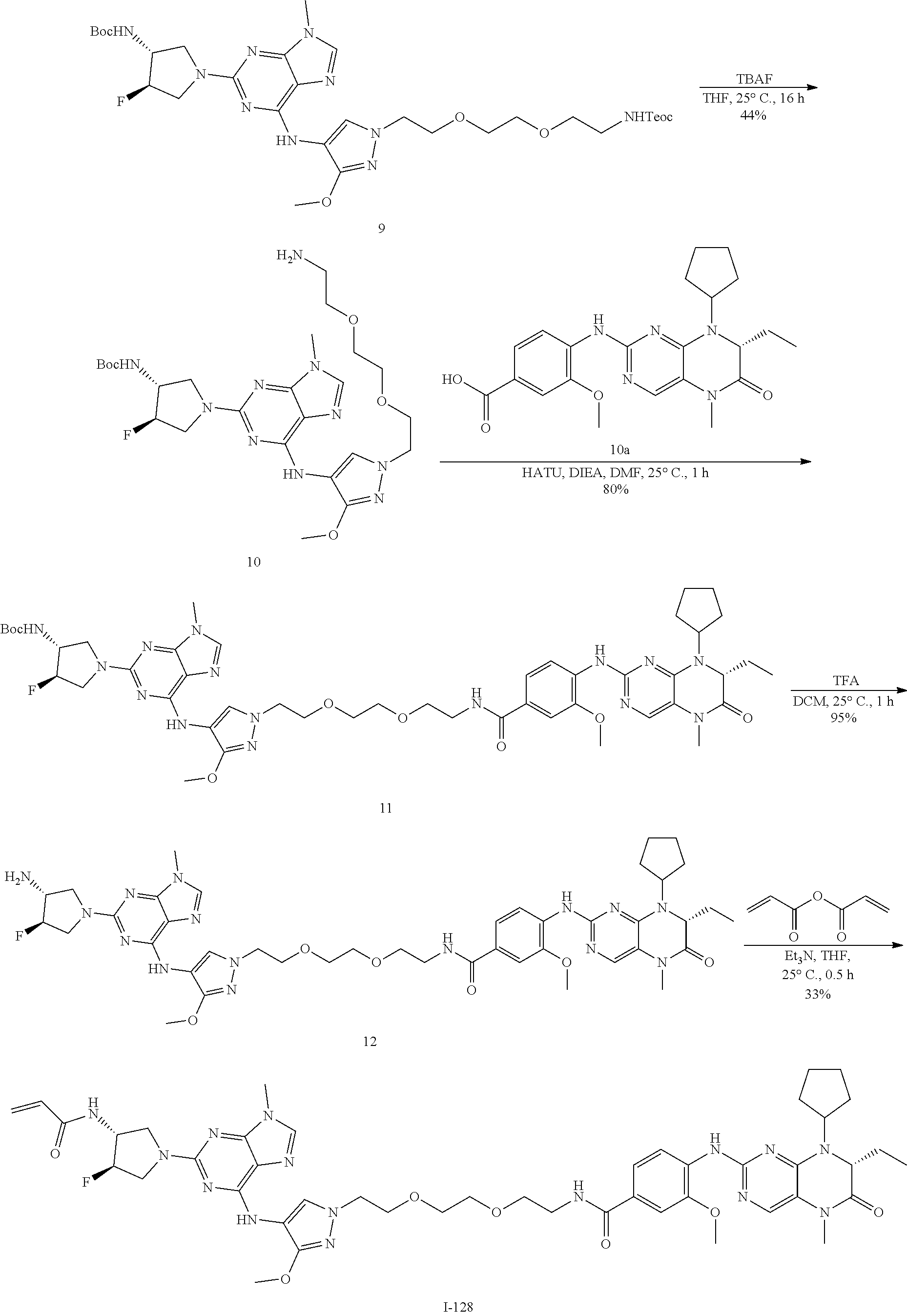

General Information: Synthetic route of compound 1 was reported by Angewandte Chemie International Edition, 2015, 10327. Synthetic route of compound 8a was reported by Journal of Medicinal Chemistry, 2017, 3002. Synthetic route of compound 10a was reported by ACS Medicinal Chemistry Letters, 2019, 1443.

Step 1. To a solution of 2-[2-(2-hydroxyethoxy)ethoxy]ethyl 4-methylbenzenesulfonate (6.00 g, 19.7 mmol, 2.2 equiv) and 3-methoxy-4-nitro-1H-pyrazole (1.30 g, 9.08 mmol, 1.0 equiv) in MeCN (40 mL) was added K₂CO₃ (2.51 g, 18.2 mmol, 2.0 equiv). The mixture solution was heated to 80° C. and stirred for 16 h. The solution was filtered by celite and the filtrate was concentrated to give a residue. The residue was purified by prep-HPLC (column: Phenomenex Synergi Max-RP 250*50 mm*10%μm; mobile phase: [water (FA)-ACN]; B%: 5%-35%, 21 min) to give 2-[2-[2-(3-methoxy-4-nitro-pyrazol-1-yl)ethoxy]ethoxy]ethanol (2.16 g, 86% yield) as a yellow oil.

Step 2. To a solution of 2-[2-[2-(3-methoxy-4-nitro-pyrazol-1-yl)ethoxy]ethoxy]ethanol (2.16 g, 7.85 mmol, 1.0 equiv) and Et₃N (2.38 g, 23.5 mmol, 3.0 equiv) in DCM (20 mL) was added TosCl (2.99 g, 15.7 mmol, 2.0 equiv). The mixture solution was stirred at 25° C. for 16 h. The solution was concentrated to give a residue. The residue was purified by silica gel chromatography (Petroleum ether: Ethyl acetate=20:1 to 2:1) to give 2-[2-[2-(3-methoxy-4-nitro-pyrazol-1-yl)ethoxy]ethoxy]ethyl 4-methylbenzenesulfonate (3.10 g, 92% yield) as a yellow oil.

Step 3. To a solution of 2-[2-[2-(3-methoxy-4-nitro-pyrazol-1-yl)ethoxy]ethoxy]ethyl 4-methylbenzenesulfonate (3.10 μg, 7.22 μmmol, 1.0 equiv) and tert-butyl N-tert-butoxycarbonylcarbamate (3.14 g, 14.4 mmol, 2.0 equiv) in MeCN (40 mL) was added K₂CO₃ (2.99 g, 21.7 mmol, 3.0 equiv). The solution was heated to 90° C. and stirred for 16 h. The solution was filtered and the filtrate was concentrated to give a residue. The residue was purified by silica gel chromatography (Petroleum ether: Ethyl acetate=1:1) to give tert-butyl N-tert-butoxycarbonyl-N-[2-[2-[2-(3-methoxy-4-nitro-pyrazol-1-yl)ethoxy]ethoxy]ethyl]carbamate (2.95 g, 86% yield) as a yellow oil. ¹H NMR (400 MHz, CDCl₃): 6 ppm 8.17 (s, 1H), 4.14 (t, J=4.8 Hz, 2H), 4.04 (s, 3H), 3.76-3.88 (m, 4H), 3.53-3.69 (m, 6H), 1.50 (s, 18H).

Step 4. To a solution of tert-butyl N-tert-butoxycarbonyl-N-[2-[2-[2-(3-methoxy-4-nitro-pyrazol-1-yl)ethoxy]ethoxy]ethyl]carbamate (2.95 g, 6.22 mmol, 1.0 equiv) in DCM (30 mL) was added TFA (10 mL). The mixture solution was stirred at 25° C. for 0.5 h. The solution was concentrated to give 2-[2-[2-(3-methoxy-4-nitro-pyrazol-1-yl)ethoxy]ethoxy]ethanamine (1.75 g, crude) as a yellow oil.

Step 5. To a solution of 2-[2-[2-(3-methoxy-4-nitro-pyrazol-1-yl)ethoxy]ethoxy]ethanamine (1.75 g, 6.38 mmol, 1.0 equiv) and Et₃N (3.23 g, 31.9 mmol, 5.0 equiv) in DCM (20 mL) was added (2,5-dioxopyrrolidin-1-yl) 2-trimethylsilylethyl carbonate (3.31 g, 12.8 mmol, 2.0 equiv). The mixture solution was stirred at 25° C. for 16 h. The solution was concentrated and purified by silica gel chromatography (Petroleum ether: Ethyl acetate=20:1 to 1:1) to give 2-trimethylsilylethyl N-[2-[2-[2-(3-methoxy-4-nitro-pyrazol-1-yl)ethoxy]ethoxy]ethyl]carbamate (2.60 g, 97% yield).

Step 6. To a solution of 2-trimethylsilylethyl N-[2-[2-[2-(3-methoxy-4-nitro-pyrazol-1-yl)ethoxy]ethoxy]ethyl]carbamate (1.30 g, 3.11 mmol, 1.0 equiv) in THF (20 mL) was added Pd/C (400 mg, 10% purity). The mixture solution was stirred at 25° C. under H₂ (15 psi, balloon) for 16 h. The solution was filtered and the filtrate was concentrated to give 2-trimethylsilylethyl N-[2-[2-[2-(4-amino-3-methoxy-pyrazol-1-yl)ethoxy]ethoxy]ethyl]carbamate (1.0 g, 83% yield) as a yellow oil.

Step 7. To a solution of 2-trimethylsilylethyl N-1[2-[2-1[2-(4-amino-3-methoxy-pyrazol-1-yl)ethoxy]ethoxy]ethyl]carbamate (1.00 g, 2.57 mmol, 1.0 equiv) and 2,6-dichloro-9-methyl-purine (678 mg, 3.34 mmol, 1.3 equiv) in i-PrOH (20 mL) was added DIEA (1.66 g, 12.8 mmol, 5.0 equiv). The mixture solution was heated to 90° C. for 16 h. The solution was concentrated and purified by silica gel chromatography (Petroleum ether: Ethyl acetate=20:1 to 1:2) to give 2-trimethylsilylethyl N-[2-[2-[2-[4-[(2-chloro-9-methyl-purin-6-yl)amino]-3-methoxy-pyrazol-1-yl]ethoxy]ethoxy]ethyl]carbamate (1.2 g, 84% yield) as a purple solid.

Step 8. To a solution of 2-trimethylsilylethyl N-[2-[2-[2-[4-[(2-chloro-9-methyl-purin-6-yl)amino]-3-methoxy-pyrazol-1-yl]ethoxy]ethoxy]ethyl]carbamate (900 mg, 1.62 mmol, 1.0 equiv) and tert-butyl N-[(3R,4R)-4-fluoropyrrolidin-3-yl]carbamate (496 mg, 2.43 mmol, 1.5 equiv) in NMP (7 mL) was added DIEA (1.05 g, 8.11 mmol, 5.0 equiv). The mixture solution was heated to 140° C. and stirred for 16 h. The solution was poured into ethyl acetate (200 mL), washed with water (200 mL×2), brine (200 mL), dried over by Na₂SO₄, filtered and the filtrate was concentrated to give a residue. The residue was purified by silica gel chromatography (Petroleum ether: Ethyl acetate=10:1 to 0:1) to give 2-trimethylsilylethyl N-[2-[2-[2-[4-[[2-[(3R,4R)-3-(tert-butoxycarbonylamino)-4-fluoro-pyrrolidin-1-yl]-9-methyl-purin-6-yl]amino]-3-methoxy-pyrazol-1-yl]ethoxy]ethoxy]ethyl]carbamate (1.10 g, 94% yield).

Step 9. To a solution of 2-trimethylsilylethyl N-[2-[2-[2-[4-[[2-[(3R,4R)-3-(tert-butoxycarbonylamino)-4-fluoro-pyrrolidin-1-yl]-9-methyl-purin-6-yl]amino]-3-methoxy-pyrazol-1-yl]ethoxy]ethoxy]ethyl]carbamate (400 mg, 553 μmol, 1.0 equiv) in THF (4 mL) was added TBAF (1 M, 1.9 mL, 3.5 equiv). The mixture solution was stirred at 25° C. for 16 h. The solution was poured into water (30 mL), extracted with ethyl acetate (30 mL×5). The combined organic layer was dried over by Na₂SO₄, filtered and the filtrate was concentrated to give a residue. The residue was purified by prep-HPLC (column: 3_Phenomenex Luna C18 75*30 mm*3 m; mobile phase: [water(TFA)-ACN]; B%: 18%-48%, 7 min) to give tert-butyl N-[(3R,4R)-1-[6-1[[1-1[2-1[2-(2-aminoethoxy)ethoxy]ethyl]-3-methoxy-pyrazol-4-yl]amino]-9-methyl-purin-2-yl]-4-fluoro-pyrrolidin-3-yl]carbamate (140 mg, 44% yield) as a yellow solid.

Step 10. To a solution of tert-butyl N-[(3R,4R)-1-[6-[[1-[2-[2-(2-aminoethoxy) ethoxy]ethyl]-3-methoxy-pyrazol-4-yl]amino]-9-methyl-purin-2-yl]-4-fluoro-pyrrolidin-3-yl]carbamate (140 mg, 241 μmol, 1.0 equiv), DIEA (156 mg, 1.21 mmol, 5.0 equiv) and 4-[[(7R)-8-cyclopentyl-7-ethyl-5-methyl-6-oxo-7H-pteridin-2-yl]amino]-3-methoxy-benzoic acid (102 mg, 241 μmol, 1.0 equiv) in DMF (3 mL) was added HATU (110 mg, 290 μmol, 1.2 equiv). The mixture solution was stirred at 25° C. for 1 h. The solution was concentrated to give a residue. The residue was purified by prep-HPLC (column: 3_Phenomenex Luna C18 75*30 mm*3 m; mobile phase: [water(TFA)-ACN]; B%: 31%-61%, 7 min) to give tert-butyl N-[(3R,4R)-1-[6-[[1-[1[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethyl]-3-methoxy-pyrazol-4-yl]amino]-9-methyl-purin-2-yl]-4-fluoro-pyrrolidin-3-yl]carbamate (190 mg, 80% yield) as a yellow solid.

Step 11. To a solution of tert-butyl N-[(3R,4R)-1-[6-[[1-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethyl]-3-methoxy-pyrazol-4-yl]amino]-9-methyl-purin-2-yl]-4-fluoro-pyrrolidin-3-yl]carbamate (70 mg, 70 μmol, 1.0 equiv) in DCM (1 mL) was added TFA (646 mg, 5.67 mmol, 0.4 mL, 80.0 equiv). The mixture solution was stirred at 25° C. for 1 h and concentrated to give N-[2-[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy]ethyl]-4-[[(7R)-8-cyclopentyl-7-ethyl-5-methyl-6-oxo-7H-pteridin-2-yl]amino]-3-methoxy-benzamide (60 mg, 95% yield).

Step 12. To a solution of N-[2-[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy]ethyl]-4-[[(7R)-8-cyclopentyl-7-ethyl-5-methyl-6-oxo-7H-pteridin-2-yl]amino]-3-methoxy-benzamide (60 mg, 67 μmol, 1.0 equiv) and Et₃N (34 mg, 0.34 mmol, 5.0 equiv) in THF (2 mL) was added prop-2-enoyl prop-2-enoate (10 mg, 75 μmol, 1.1 equiv). The mixture solution was stirred at 25° C. for 0.5 h. The solution was concentrated and purified by prep-HPLC (column: Unisil 3-100 C18 Ultra 150*50 mm*3 m; mobile phase: [water(FA)-ACN]; B%: 15%-45%, 10 min) to give 4-[[(7R)-8-cyclopentyl-7-ethyl-5-methyl-6-oxo-7H-pteridin-2-yl]amino]-N-[2[22-[2-[4-[[2-[(3R,4R)-3-fluoro-4-(prop-2-enoylamino)pyrrolidin-1-yl]-9-methyl-purin-6-yl]amino]-3-methoxy-pyrazol-1-yl]ethoxy]ethoxy]ethyl]-3-methoxy-benzamide (21 mg, 33% yield) as a white solid. ¹H NMR (400 MHz, DMSO-d₆): δ8.49 (d, J=6.4 Hz, 1H), 8.43-8.37 (m, 2H), 8.02 (s, 1H), 7.89 (s, 1H), 7.84 (s, 1H), 7.78 (s, 1H), 7.59 (s, 1H), 7.51-7.46 (m, 2H), 6.34-6.05 (m, 2H), 5.61 (m, J=2.4, 9.6 Hz, 1H), 5.22-4.99 (m, 1H), 4.52-4.41 (m, 1H), 4.37-4.29 (m, 1H), 4.23 (m, J=3.6, 7.6 Hz, 1H), 4.09 (t, J=5.2 Hz, 2H), 3.92 (s, 3H), 3.87-3.69 (m, 9H), 3.68-3.60 (m, 4H), 3.55-3.47 (m, 7H), 3.24 (s, 3H), 2.06-1.97 (m, 1H), 1.88 (s, 2H), 1.81-1.72 (m, 4H), 1.68-1.57 (m, 3H), 0.76 (t, J=7.2 Hz, 3H). LC-MS: MS (ES⁺): RT=2.186 min, m/z=941.3 [M+H⁺]; LCMS method: 10.

### Example 113—Synthesis of Compound I-129

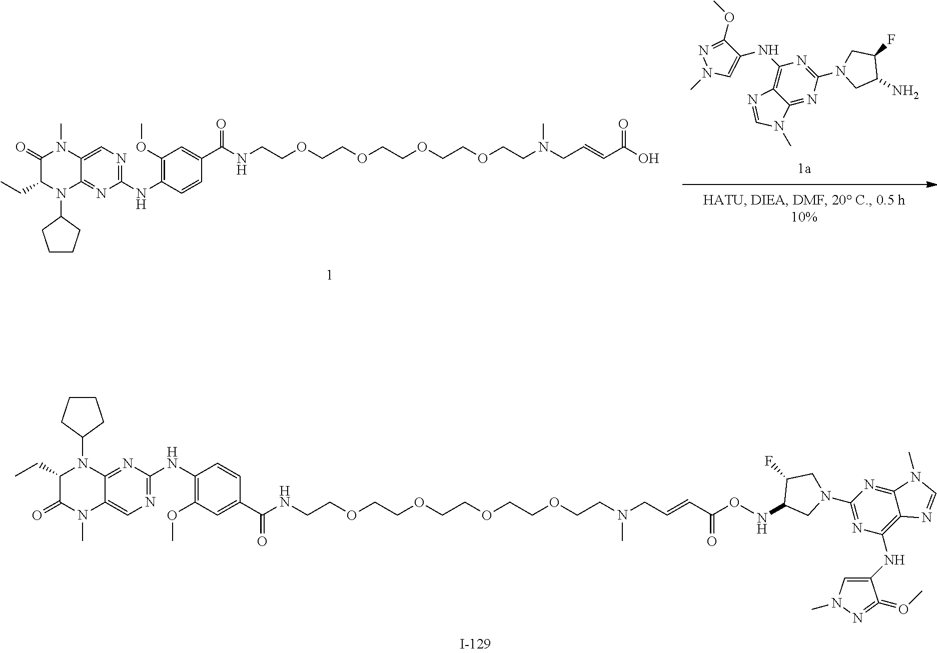

To a solution of (E)-4-[2-[2-[2-[2-[2-[[4-[[(7R)-8-cyclopentyl-7-ethyl-5-methyl-6-oxo-7H-pteridin-2-yl]amino]-3-methoxy-benzoyl]amino]ethoxy]ethoxy]ethoxy]ethoxy]ethyl-methyl-amino]but-2-enoic acid (35 mg, 47 μmol, 1.0 equiv) in DMF (1 mL) was added 2-[(3R,4R)-3-amino-4-fluoro-pyrrolidin-1-yl]-N-(3-methoxy-1-methyl-pyrazol-4-yl)-9-methyl-purin-6-amine (19 mg, 47 μmol, 1.0 equiv, HCl) and DIEA (31 mg, 236 μmol, 5.0 equiv) and HATU (23 mg, 61 μmol, 1.3 equiv).The mixture was stirred at 20° C. for 0.5 h and concentrated. The residue was purified by prep-HPLC (column: Phenomenex C18 75*30 mm*3 m; mobile phase: [water(FA)-ACN]; B%: 12%-42%, 7 min) to give 4-[[(7R)-8-cyclopentyl-7-ethyl-5-methyl-6-oxo-7H-pteridin-2-yl]amino]-N-[2-[2-[2-[2-[2-[[(E)-4-[[(3R,4R)-4-fluoro-1-[6-[(3-methoxy-1-methyl-pyrazol-4-yl)amino]-9-methyl-purin-2-yl]pyrrolidin-3-yl]amino]-4-oxo-but-2-enyl]-methyl-amino]ethoxy]ethoxy]ethoxy]ethoxy]ethyl]-3-methoxy-benzamide (5 mg, 10% yield). ¹H NMR (400 MHz, MeOD) δ8.43-8.39 (m, 1H), 7.94 (s, 1H), 7.76-7.69 (m, 2H), 7.48-7.42 (m, 2H), 6.90-6.82 (m, 1H), 6.26-6.20 (m, 1H), 5.29-5.13 (m, 1H), 4.76-4.58 (m, 3H), 4.51-4.41 (m, 1H), 4.31-4.26 (m, 1H), 4.04-3.86 (m, 9H), 3.78-3.43 (m, 24H), 3.31 (s, 4H), 2.68-2.58 (m, 2H), 2.34 (s, 3H), 2.23-2.15 (m, 1H), 2.10-1.99 (m, 1H), 1.97-1.84 (m, 4H), 1.80-1.68 (m, 4H), 0.88-0.81 (m, 3H). LC-MS: MS (ES⁺): RT=2.040 min, m/z=1085.5 [M+H⁺]; LCMS Method: 10.

### Example 114—Synthesis of Compound I-130

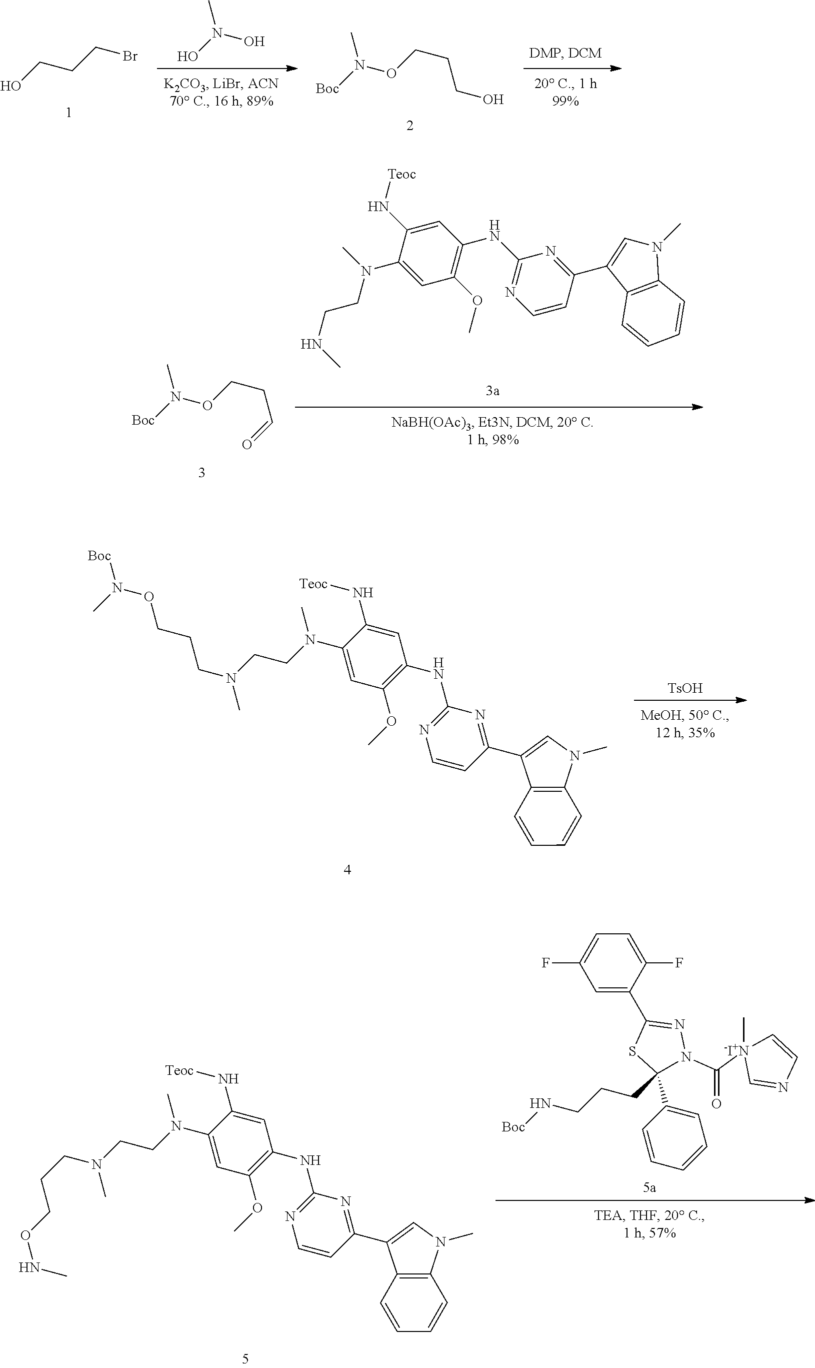

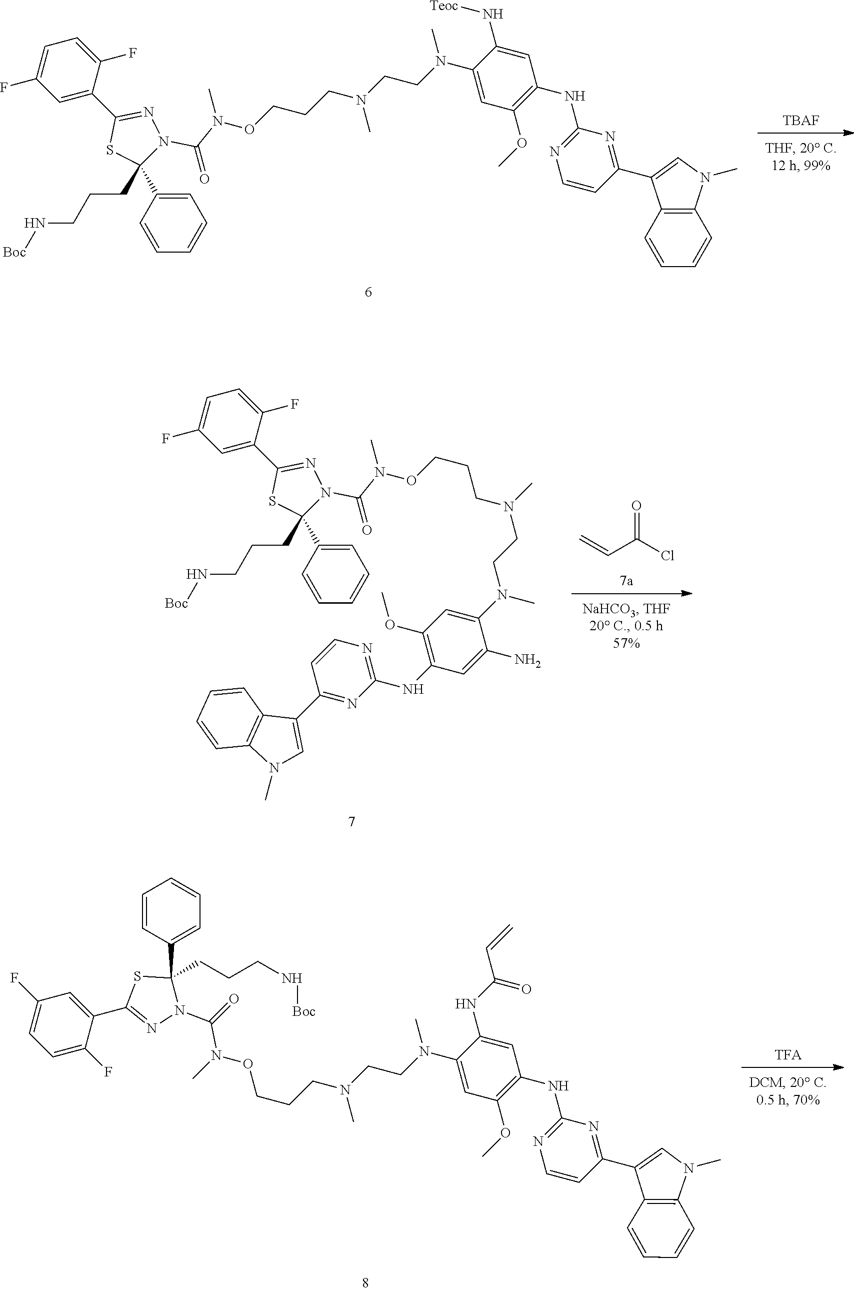

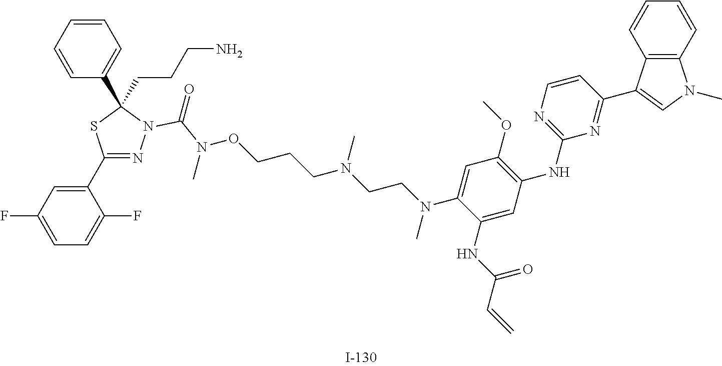 [SMILES: C=CC(=O)Nc1cc(Nc2nccc(-c3cn(C)c4ccccc34)n2)c(OC)cc1N(C)CCN(C)CCCON(C)C(=O)N1N=C(c2cc(F)ccc2F)S[C@@]1(CCCN)c1ccccc1]

General Information: Synthetic route of compound 5a is a known compound from WO2006/44825, 2006, A2.

Step 1. To a solution of 3-bromopropan-1-ol (519.4 mg, 3.7 mmol, 337.3 μL, 1.1 equiv) and tert-butyl N-hydroxy-N-methyl-carbamate (500 mg, 3.4 mmol, 1.0 equiv) in ACN (8 mL) was added K₂CO₃ (939 mg, 6.8 mmol, 2.0 equiv) and LiBr (5.9 mg, 68 μmol, 0.02 equiv). The mixture was stirred at 70° C. for 16 h. The reaction mixture was concentrated. The residue was purified by column chromatography (SiO₂, Petroleum ether/Ethyl acetate=10/1 to 2/1) to give tert-butyl N-(3-hydroxypropoxy)-N-methyl-carbamate (620 mg, 89% yield).

Step 2. To a solution of tert-butyl N-(3-hydroxypropoxy)-N-methyl-carbamate (50 mg, 244 μmol, 1.0 equiv) in DMF (0.5 mL) was added DMP (258 mg, 609 μmol, 2.5 equiv).The mixture was stirred at 20° C. for 1 h. The reaction mixture was concentrated. No purification. The product was used directly for next step.

Step 3. To a solution of 2-trimethylsilylethyl N-[4-methoxy-5-[[4-(1-methylindol-3-yl)pyrimidin-2-yl]amino]-2-[methyl-[2-(methylamino)ethyl]amino]phenyl]carbamate (140 mg, 243 μmol, 1.0 equiv) in DCM (5 mL) was added NaBH(OAc)₃ (516 mg, 2.44 mmol, 10.0 equiv) and Et3N (246 mg, 2.4 mmol, 10 equiv) then tert-butyl N-methyl-N-(3-oxopropoxy)carbamate (50 mg, 244 μmol, 1.0 equiv) was added. The mixture was stirred at 20° C. for 1 h and concentrated. The residue was purified by prep-HPLC (FA condition) to give tert-butyl N-[3-[2-[5-methoxy-N-methyl-4-[[4-(1-methylindol-3-yl)pyrimidin-2-yl]amino]-2-(2trimethylsilylethoxycarbonyl amino)anilino]ethyl-methyl-amino]propoxy]-N-methyl-carbamate (180 mg, 97% yield).

Step 4. To a solution of tert-butyl N-[3-[2-[5-methoxy-N-methyl-4-[[4-(1-methylindol-3-yl)pyrimidin-2-yl]amino]-2-(2-trimethylsilylethoxycarbonylamino)anilino]ethyl-methyl-amino]propoxy]-N-methyl-carbamate (200 mg, 262 μmol, 1.0 equiv) in MeOH (10 mL) was added TsOH (90 mg, 524 μmol, 2.0 equiv). The mixture was stirred at 50° C. for 12 h and concentrated. The residue was purified by prep-HPLC (column: Phenomenex C18 75*30 mm*3 m; mobile phase: [water(FA)-ACN]; B%: 18%-48%, 7 min) to give 2-trimethylsilylethyl N-[4-methoxy-5-[[4-(1-methylindol-3-yl)pyrimidin-2-yl]amino]-2-[methyl-[2-[methyl-[3-(methylaminooxy) propyl]amino]ethyl]amino]phenyl]carbamate (60 mg, 35% yield).

Step 5. To a solution of [4-methoxy-5-[[4-(1-methylindol-3-yl)pyrimidin-2-yl]amino]-2-[methyl-[2-[methyl-[3-(methylaminooxy)propyl]amino]ethyl]amino]anilino]3-trimethylsilyl propanoate (100 mg, 150 μmol, 1.0 equiv) in THF (4 mL) was added TEA (46 mg, 452 μmol, 3.0 equiv) and tert-butyl N-[3-[(2S)-5-(2,5-difluorophenyl)-3-(3-iodo-3-methyl-imidazole-1-carbonyl)-2-phenyl-1,3,4-thiadiazol-2-yl]propyl]carbamate (131 mg, 196 μmol, 1.3 equiv). The mixture was stirred at 20° C. for 0.5 h and concentrated. The residue was purified by prep-TLC (SiO₂, DCM: MeOH=30:1) to give [2-[2-[3-[[(2S)-2-[3-(tert-butoxycarbonylamino)propyl]-5-(2,5-difluorophenyl)-2-phenyl-1,3,4-thiadiazole-3-carbonyl]-methyl-amino]oxypropyl-methyl-amino]ethyl-methyl-amino]-4-methoxy-5-[[4-(1-methylindol-3-yl)pyrimidin-2-yl]amino]anilino]3-trimethylsilylpropanoate (100 mg, 57% yield).

Step 6. A mixture of [2-[2-[3-[[(2S)-2-[3-(tert-butoxycarbonylamino)propyl]-5-(2,5-difluorophenyl)-2-phenyl-1,3,4-thiadiazole-3-carbonyl]-methyl-amino]oxypropyl-methyl-amino]ethyl-methyl-amino]-4-methoxy-5-[[4-(1-methylindol-3-yl)pyrimidin-2-yl]amino]anilino]3-trimethylsilylpropanoate (100 mg, 90 μmol, 1.0 equiv) and TBAF (1.0 M, 267 μL, 3.0 equiv) in THF (3 mL) was degassed and purged with N₂ for 3 times, and then the mixture was stirred at 20° C. for 12 h under N₂ atmosphere. The mixture was concentrated. The product was used directly for next step.

Step 7. To a solution of tert-butyl N—[3-[(2S)-3-[3-[2-[2-amino-5-methoxy-N-methyl-4-[[4-(1-methylindol-3-yl)pyrimidin-2-yl]amino]anilino]ethyl-methyl-amino]propoxy-methyl-carbamoyl]-5-(2,5-difluorophenyl)-2-phenyl-1,3,4-thiadiazol-2-yl]propyl]carbamate (87 mg, 89 μmol, 1.0 equiv) in THF (4 mL) was added NaHCO₃ (22 mg, 267 μmol, 3.0 equiv) and prop-2-enoyl prop-2-enoate (11 mg, 89 μmol, 1.0 equiv). The mixture was stirred at 20° C. for 0.5 h. The reaction was quenched by addition of 10 mL of H₂O and then extracted by DCM (3×10 mL). The residue was purified by prep-TLC (SiO₂, DCM: MeOH=10:1) to give tert-butyl N-[3-[(2S)-5-(2,5-difluorophenyl)-3-[3-[2-[5-methoxy-N-methyl-4-1[[4-(1-methylindol-3-yl)pyrimidin-2-yl]amino]-2-(prop-2-enoylamino)anilino]ethyl-methyl-amino]propoxy-methyl-carbamoyl]-2-phenyl-1,3,4-thiadiazol-2-yl]propyl]carbamate (53 mg, 57% yield).

Step 8. To a solution of tert-butyl N-[3-[(2S)-5-(2,5-difluorophenyl)-3-[3-[2-[5-methoxy-N-methyl-4-[[4-(1-methylindol-3-yl)pyrimidin-2-yl]amino]-2-(prop-2-enoylamino)anilino]ethyl-methyl-amino]propoxy-methyl-carbamoyl]-2-phenyl-1,3,4-thiadiazol-2-yl]propyl]carbamate (50 mg, 48 μmol, 1.0 equiv) in DCM (3 mL) was added TFA (500 μL). The mixture was stirred at 20° C. for 0.5 h and concentrated. The residue was purified by prep-HPLC(column: Unisil 3-100 C18 Ultra 150*50 mm*3 m; mobile phase: [water(FA)-ACN]; B%: 10%-40%, 10 min) to give (2S)-2-(3-aminopropyl)-5-(2,5-difluorophenyl)-N-[3-[2-[5-methoxy-N-methyl-4-[[4-(1-methylindol-3-yl)pyrimidin-2-yl]amino]-2-(prop-2-enoylamino)anilino]ethyl-methyl-amino]propoxy]-N-methyl-2-phenyl-1,3,4-thiadiazole-3-carboxamide (33.2 mg, 70% yield). ¹H NMR (400 MHz, MeOD) δ8.93 (s, 1H), 8.52 (s, 1H), 8.37-8.28 (m, 3H), 7.75 (s, 1H), 7.47 (s, 3H), 7.36 (t, J=7.2 Hz, 2H), 7.31-7.17 (m, 4H), 7.14-7.01 (m, 2H), 6.96 (s, 1H), 6.59-6.39 (m, 2H), 5.83 (s, 1H), 4.04-3.97 (m, 4H), 3.96-3.89 (m, 4H), 3.30-3.26 (m, 2H), 3.14 (s, 3H), 3.10-2.92 (m, 7H), 2.70 (s, 3H), 2.63-2.49 (m, 4H), 2.16-2.04 (m, 1H), 1.87 (s, 2H), 1.77-1.68 (m, 1H). LC-MS: MS (ES⁺): RT=2.188 min, m/z=932.4 [M+H⁺]; LCMS method: 25.

### Example 115—Synthesis of Compound I-131

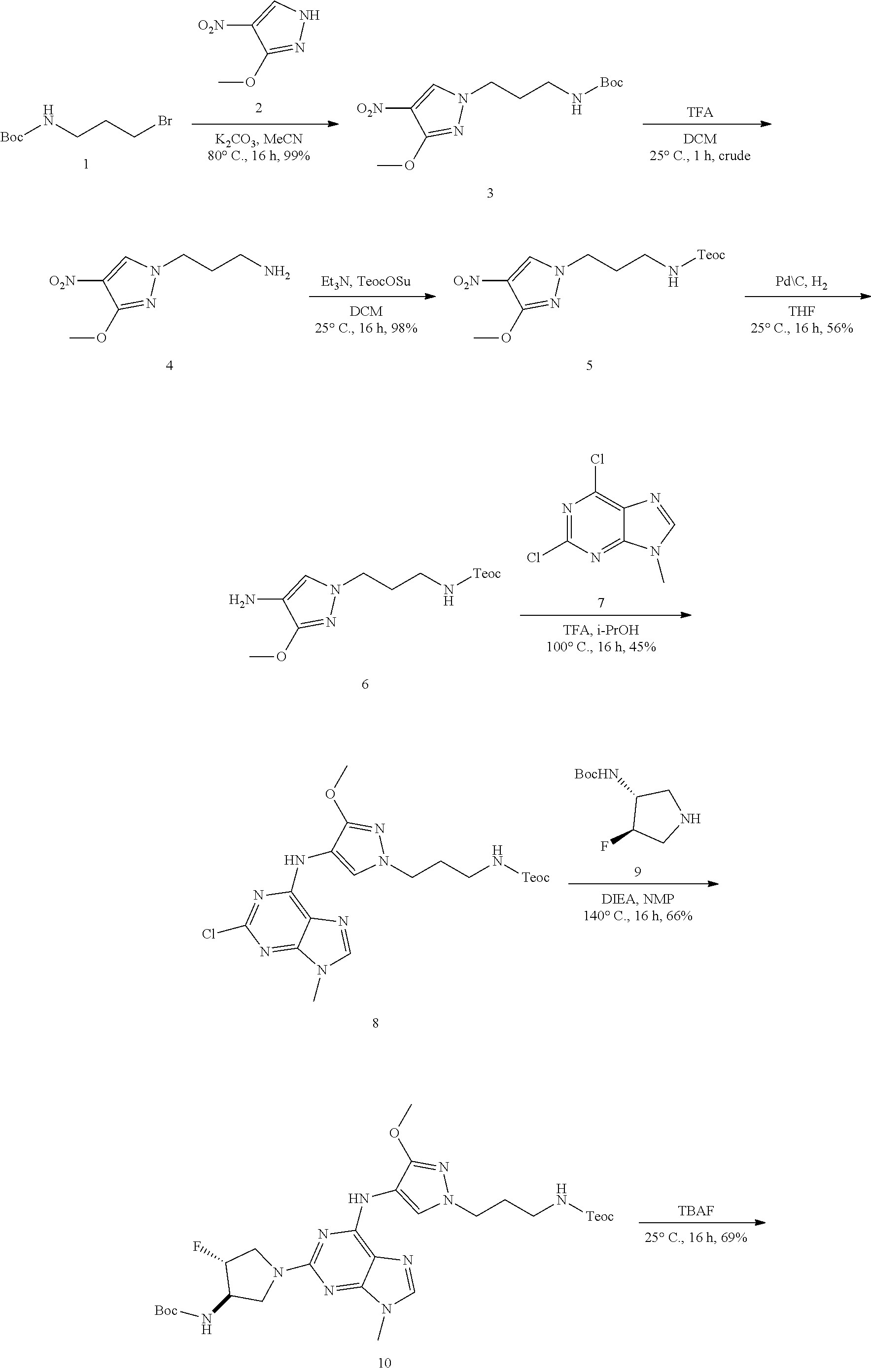

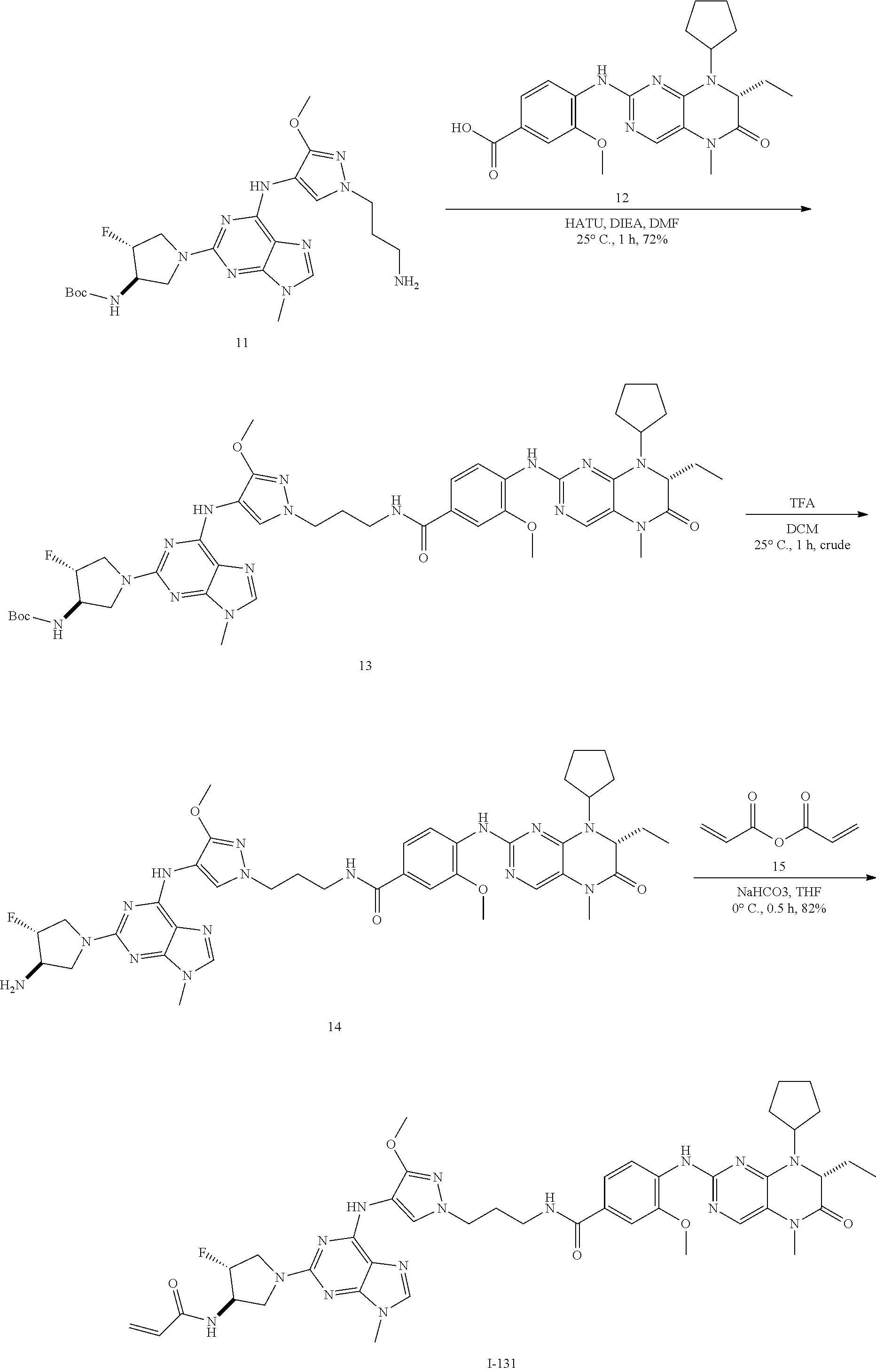

General Information: Compound 7 is a known compound from WO2014/189830. Compound 9 is a known compound from Journal of Medicinal Chemistry, 2017, vol. 60, # 7, p. 3002-3019. Compound 12 is a known compound from WO2017/24317.

Step 1. To a solution of tert-butyl N-(3-bromopropyl)carbamate (2.4 g, 10 mmol, 1.2 equiv) and 3-methoxy-4-nitro-1H-pyrazole (1.2 g, 8.39 mmol, 1 equiv) in MeCN (15 mL) was added K₂CO₃ (2.32 g, 16.77 mmol, 2 equiv), the mixture was stirred at 80° C. for 16 h. The mixture was filtered and concentrated. The residue was purified by column chromatography (SiO₂, Petroleum ether/Ethyl acetate=10/1 to 2/1) to give tert-butyl N-[3-(3-methoxy-4-nitro-pyrazol-1-yl)propyl]carbamate (2.5 g, 99% yield).

Step 2. To a solution of tert-butyl N-[3-(3-methoxy-4-nitro-pyrazol-1-yl)propyl]carbamate (2.4 g, 8 mmol, 1 equiv) in DCM (15 mL) was added TFA (3 mL), the mixture was stirred at 25° C. for 1 h. The crude product was used into the next step without further purification.

Step 3. To a solution of 3-(3-methoxy-4-nitro-pyrazol-1-yl)propan-1-amine (2.5 g, 7.96 mmol, 1 equiv, TFA) and TEA (4 g, 40 mmol, 6 mL, 5 equiv) in DCM (30 mL) was added (2,5-dioxopyrrolidin-1-yl) 2-trimethylsilylethyl carbonate (4.13 g, 15.91 mmol, 2 equiv), the mixture was stirred at 25° C. for 16 h. The mixture was concentrated to give a residue. The residue was purified by column chromatography (SiO₂, Petroleum ether/Ethyl acetate=10/1 to 1/1) to give 2-trimethylsilylethyl N-[3-(3-methoxy -4-nitro -pyrazol-1-yl)propyl]carbamate (2.7 g, 98% yield). ¹H NMR (400 MHz, CDCl₃): δ8.21-8.01 (m, 1H), 5.00-4.77 (m, 1H), 4.23-4.05 (m, 4H), 4.03 (s, 3H), 3.33-3.10 (m, 2H), 2.16-2.05 (m, 2H), 1.05-0.87 (m, 2H), 0.04 (s, 9H).

Step 4. To a solution of 2-trimethylsilylethyl N-[3-(3-methoxy-4-nitro-pyrazol-1-yl)propyl]carbamate (1.3 g, 3.77 mmol, 1 equiv) in THF (20 mL) was added Pd/C (300 mg, 10% purity) under N₂. The mixture was stirred under H₂ (15 psi) at 25° C. for 16 h. The mixture was filtered and concentrated. The residue was purified by column chromatography (SiO₂, Petroleum ether/Ethyl acetate=5/1 to 0/1) to give 2-trimethylsilylethyl N-[3-(4-amino-3-methoxy-pyrazol-1-yl)propyl]carbamate (660 mg, 56% yield).

Step 5. To a solution of 2-trimethylsilylethyl N-[3-(4-amino-3-methoxy-pyrazol-1-yl)propyl]carbamate (660 mg, 2.10 mmol, 1 equiv) and 2,6-dichloro-9-methyl-purine (426 mg, 2.10 mmol, 1 equiv) in i-PrOH (10 mL) was added TFA (478 mg, 4.20 mmol, 311 μL, 2 equiv). The mixture was stirred at 100° C. for 16 h. The mixture was concentrated. The residue was purified by prep-HPLC (column: Phenomenex luna C18 150*40 mm*15 m; mobile phase: [water(TFA)-ACN]; B%: 43%-73%, 10 min) to give 2-trimethylsilylethyl N-[3-[4-[(2-chloro-9-methyl-purin-6-yl) amino]-3-methoxy-pyrazol-1-yl]propyl]carbamate (460 mg, 46% yield).

Step 6. To a solution of 2-trimethylsilylethyl N-[3-[4-[(2-chloro-9-methyl-purin-6-yl)amino]-3-methoxy-pyrazol -1-yl]propyl]carbamate (460 mg, 956 μmol, 1 equiv) and tert-butyl N-[(3R,4R)-4-fluoropyrrolidin-3-yl]carbamate (293 mg, 1.43 mmol, 1.5 equiv) in NMP (3 mL) was added DIEA (618 mg, 4.78 mmol, 5 equiv). The mixture was stirred at 140° C. for 16 h. The mixture was concentrated. The residue was purified by prep-HPLC (column: Waters Xbridge C18 150*50 mm*10 m; mobile phase: [water(NH4HCO₃)—ACN]; B%: 45%-75%, 11 min) to give 2-trimethylsilylethyl N-[3-[4-[[2-[(3R,4R)-3-(tert-butoxycarbonylamino)-4-fluoro-pyrrolidin-1-yl]-9-methyl-purin-6-yl]amino]-3-methoxy-pyrazol-1-yl]propyl]carbamate (410 mg, 66% yield). ¹H NMR (400 MHz, DMSO-d₆): δ8.05-8.01 (m, 1H), 7.92-7.84 (m, 1H), 7.82-7.73 (m, 1H), 7.38-7.25 (m, 1H), 7.13-7.03 (m, 1H), 5.17-4.96 (m, 1H), 4.17-4.07 (m, 1H), 4.06-4.00 (m, 2H), 3.99-3.92 (m, 2H), 3.82 (s, 3H), 3.81-3.69 (m, 3H), 3.60 (s, 3H), 3.58-3.52 (m, 1H), 3.03-2.93 (m, 2H), 1.90-1.79 (m, 2H), 1.39 (s, 9H), 0.95-0.85 (m, 2H), 0.01 (s, 9H).

Step 7. A solution of 2-trimethylsilylethyl N-[3-[4-[[2-[(3R,4R)-3-(tert-butoxycarbonylamino)-4-fluoro-pyrrolidin-1-yl]-9-methyl-purin-6-yl]amino]-3-methoxy-pyrazol-1-yl]propyl]carbamate (410 mg, 632 μmol, 1 equiv) in TBAF (1 M, 2 mL, 3 equiv) was stirred at 25° C. for 16 h. The mixture was diluted with water (70 mL) and extracted with EtOAc (20 mL×8). The combined organic layers were concentrated. The residue was purified by prep-HPLC (column: Phenomenex luna C18 150*40 mm*15 m; mobile phase: [water(TFA)-ACN]; B%: 9%-39%, 10 min) to give tert-butyl N-[(3R,4R)-1-[6-[[1-(3-aminopropyl)-3-methoxy-pyrazol-4-yl]amino]-9-methyl-purin-2-yl]-4-fluoropyrrolidin-3-yl]carbamate (220 mg, 69% yield).

Step 8. To a solution of tert-butyl N-[(3R,4R)-1-[6-[[1-(3-aminopropyl)-3-methoxy-pyrazol-4-yl]amino]-9-methyl -purin-2-yl]-4-fluoropyrrolidin-3-yl]carbamate (100 mg, 198 μmol, 1 equiv) and 4-[(8-cyclopentyl-7-ethyl -5-methyl-6-oxo-7H-pteridin-2-yl)amino]-3-methoxy-benzoic acid (84 mg, 198 μmol, 1 equiv) in DMF (2 mL) was added DIEA (77 mg, 594 μmol, 3 equiv) and HATU (90 mg, 238 μmol, 1.2 equiv), the mixture was stirred at 25° C. for 1 h. The mixture was concentrated to give a residue. The residue was purified by prep-HPLC (column: Unisil 3-100 C18 Ultra 150*50 mm*3 m; mobile phase: [water(FA)-ACN]; B%: 25%-55%, 10 min) to give tert-butyl N-[(3R,4R)-1-[6-[[1-[3-[[4-[(8-cyclopentyl-7-ethyl-5-methyl-6-oxo-7H-pteridin-2-yl)amino]-3-methoxybenzoyl]amino]propyl]-3-methoxy-pyrazol-4-yl]amino]-9-methyl-purin-2-yl]-4-fluoro-pyrrolidin-3-yl]carbamate (130 mg, 72% yield).

Step 9. To a solution of tert-butyl N-[(3R, 4R)-1-[6-[[1-[3-[[4-[(8-cyclopentyl-7-ethyl-5-methyl-6-oxo-7H-pteridin-2-yl)amino]-3-methoxy-benzoyl]amino]propyl]-3-methoxy-pyrazol-4-yl]amino]-9-methyl-purin-2-yl]-4-fluoro-pyrrolidin-3-yl]carbamate (130 mg, 142 μmol, 1 equiv) in DCM (3 mL) was added TFA (500 μL), the mixture was stirred at 25° C. for 1 h. The crude product was used into the next step without further purification.

Step 10. To a solution of N-[3-[4-[[2-[(3R,4R)-3-amino-4-fluoro-pyrrolidin-1-yl]-9-methyl-purin-6-yl]amino]-3-methoxy-pyrazol-1-yl]propyl]-4-[(8-cyclopentyl-7-ethyl-5-methyl-6-oxo-7H-pteridin-2-yl)amino]-3-methoxy-benzamide (130 mg, 140 μmol, 1 equiv, TFA salt) and NaHCO₃ (1 M, 3 mL, 21 equiv) in THF (9 mL) was added prop-2-enoyl prop-2-enoate (18 mg, 140 μmol, 1 equiv) at 0° C., the mixture was stirred at 0° C. for 0.5 h. The mixture was diluted with water (20 mL) and extracted with EtOAc (30 mL×3). The combined organic layers were washed with brine (30 mL), dried over Na₂SO₄, filtered and concentrated. The residue was purified by prep-HPLC (column: Phenomenex luna C18 250*50 mm*15 m; mobile phase: [water(FA)-ACN]; B%: 2%-53%, 10 min) to give 4-[(8-cyclopentyl-7-ethyl-5-methyl-6-oxo-7H-pteridin-2-yl)amino]-N-[3-[4-[[2-[(3R,4R)-3-fluoro-4-(prop-2-enoylamino)pyrrolidin-1-yl]-9-methyl-purin-6-yl]amino]-3-methoxy-pyrazol-1-yl]propyl]-3-methoxy-benzamide (100 mg, 82% yield). ¹H NMR (400 MHz, MeOD): δ8.35 (d, J=8.4 Hz, 1H), 8.09 (s, 1H), 7.80 (s, 1H), 7.65 (s, 1H), 7.38-7.27 (m, 2H), 6.28 (d, J=6.0 Hz, 2H), 5.66 (t, J=6.0 Hz, 1H), 5.27-5.09 (m, 1H), 4.66-4.44 (m, 2H), 4.31 (m, 1H), 4.15 (t, J=6.4 Hz, 2H), 4.05-3.81 (m, 9H), 3.65 (s, 3H), 3.51 (t, J=6.0 Hz, 2H), 3.38-3.35 (m, 4H), 2.23-2.13 (m, 3H), 2.02-1.71 (m, 9H), 0.88 (t, J=7.6 Hz, 3H). LC-MS: MS (ES′): RT=2.181 min, m/z=866.4 [M+H⁺]; LCMS method: 10.

### Example 116—Synthesis of Compound I-132

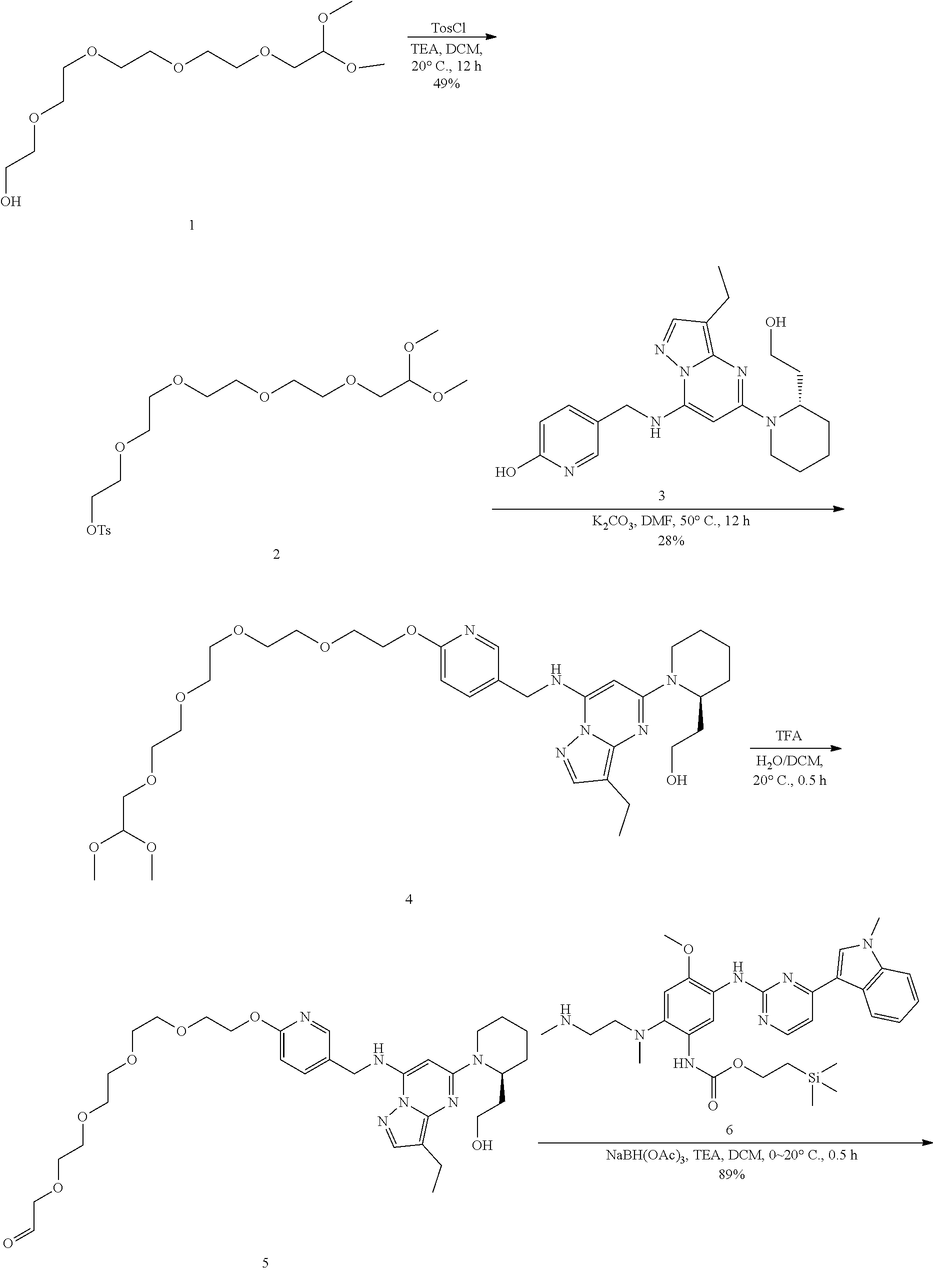

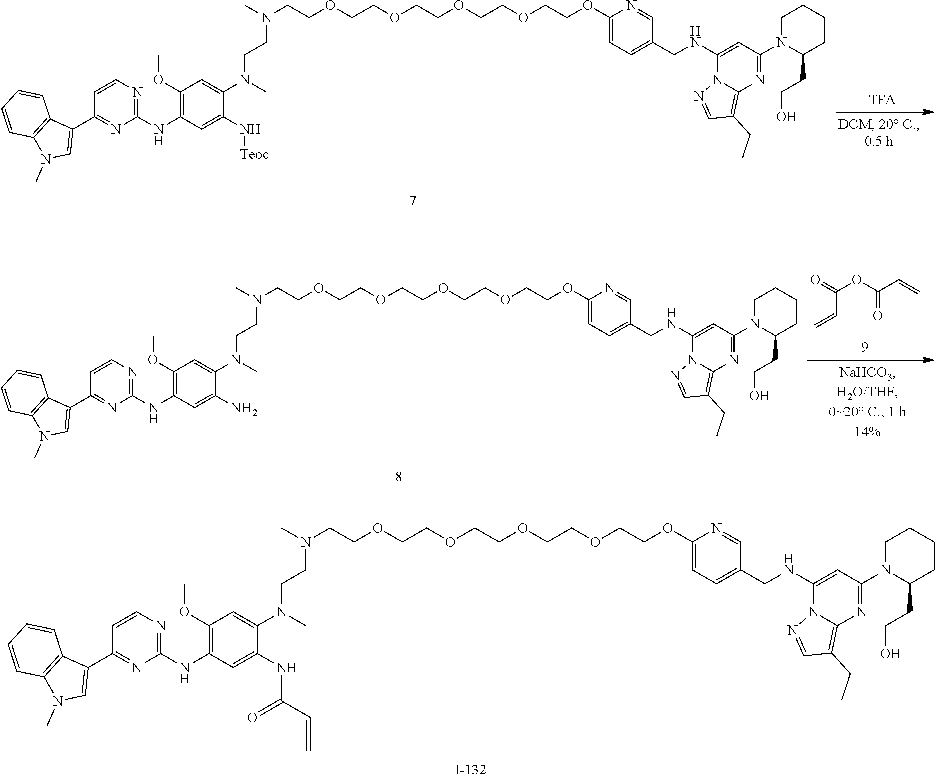

Step 1. To a solution of compound 1 (250 mg, 886 μmol, 1.0 equiv) in DCM (2 mL) was added TEA (314 mg, 3.10 mmol, 431 μL, 3.5 equiv) and TosCl (422 mg, 2.21 mmol, 2.5 equiv). The mixture was stirred at 20° C. for 12 h. The reaction mixture was concentrated under reduced pressure to give a residue. The residue was purified by prep-HPLC (column: Waters Xbridge 150*25 mm*5 m; mobile phase: [water (NH₄HCO₃)-ACN]; B%: 25%-55%, 10 min) to afford compound 2 (190 mg, 435 μmol, 49% yield) as a yellow oil. ¹H NMR: (400 MHz, CDCl₃) δ7.84-7.76 (m, 2H), 7.35 (d, J=8.0 Hz, 2H), 4.56-4.48 (m, 1H), 4.22-4.13 (m, 2H), 3.68-3.62 (m, 9H), 3.59 (s, 4H), 3.56-3.52 (m, 2H), 3.41-3.38 (m, 6H), 2.48-2.44 (m, 3H).

Step 2. To a solution of compound 2 (190 mg, 435 μmol, 1.0 equiv), compound 3 (173 mg, 435 μmol, 1.0 equiv) in DMF (3 mL) was added K₂CO₃ (60.1 mg, 435 μmol, 1.0 equiv). The mixture was stirred at 50° C. for 12 h. The reaction mixture was filtered and concentrated under reduced pressure to give a residue. The residue was purified by prep-HPLC (column: Waters Xbridge 150*25 mm*5 m; mobile phase: [water (NH₄HCO₃)-ACN]; B%: 36%-66%, 10 min) to afford compound 4 (80.0 mg, 120 μmol, 28% yield) as a yellow oil.

Step 3. To a solution of compound 4 (80.0 mg, 121 μmol, 1.0 equiv) in DCM (1 mL) was added TFA (0.02 mL) and H₂O (0.1 mL). The mixture was stirred at 20° C. for 0.5 h. The reaction mixture was filtered and concentrated under reduced pressure to give compound 5 (74.42 mg, crude) as a brown liquid and used into next step directly.

Step 4. To a solution of compound 5 (74.0 mg, 120 μmol, 1.0 equiv) and TEA (146 mg, 1.44 mmol, 201 μL, 12 equiv) in DCM (0.5 mL) was added NaBH(OAc)₃ (255 mg, 1.20 mmol, 10 equiv), compound 6 (69.3 mg, 120 μmol, 1.0 equiv) at 0° C. The mixture was stirred 20° C. for 0.5 h. The reaction mixture was diluted with 10 mL H₂O and extracted with DCM (10 mL×2). The combined organic layers were dried over Na₂SO₄, filtered and concentrated under reduced pressure to give a residue. The residue was purified by prep-HPLC (column: 3_Phenomenex Luna C18 75*30 mm*3 m; mobile phase: [water (TFA)-ACN]; B%: 32%-62%, 7 min) to afford compound 7 (128 mg, 107 μmol, 89% yield) as a yellow oil.

Step 5. To a solution of compound 7 (65 mg, 55 μmol, 1.0 equiv) in DCM (1 mL) was added TFA (0.5 mL). The mixture was stirred at 20° C. for 0.5 h. The reaction mixture was concentrated under reduced pressure to give a residue to afford compound 8 (63 mg, crude, TFA salt) as a yellow solid and used into next step directly.

Step 6. To a solution of compound 8 (63 mg, 55 μmol, 1.0 equiv, TFA salt) in THF (1 mL) and NaHCO₃ (216 mg, 2.57 mmol, 46.7 equiv) in H₂O (1 mL) was added compound 9 (6.9 mg, 55 μmol, 1.0 equiv) at 0° C. The mixture was stirred at 20° C. for 1 h. The reaction mixture was diluted with H₂O (10 mL) and extracted with EA (10 mL×2). The combined organic layers were dried over Na₂SO₄, filtered and concentrated under reduced pressure to give a residue. The residue was purified by prep-HPLC (column: Waters Xbridge 150*25 mm*5 m; mobile phase: [water (NH₄HCO₃)-ACN]; B%: 64%-94%, 8 min) to afford compound I-132 (9 mg, 8 μmol, 14% yield) as a yellow solid. LC-MS: MS (ES⁺): RT=1.788 min, m/z=542.8 [M/2+H]+; LCMS method: 25. ¹H NMR: (400 MHz, CD₃OD) (9.57-9.48 (m, 1H), 8.73-8.64 (m, 1H), 8.32-8.23 (m, 1H), 8.17-8.08 (m, 2H), 7.70-7.59 (m, 2H), 7.48-7.39 (m, 1H), 7.27-7.14 (m, 3H), 7.00-6.93 (m, 1H), 6.77-6.61 (m, 2H), 6.45-6.34 (m, 1H), 5.73 (s, 1H), 5.43 (s, 1H), 4.79-4.74 (m, 2H), 4.46 (s, 2H), 4.33-4.27 (m, 2H), 3.95-3.91 (m, 6H), 3.72-3.67 (m, 2H), 3.53-3.50 (m, 4H), 3.43 (d, J=8.9 Hz, 9H), 3.04-2.89 (m, 4H), 2.72-2.68 (m, 3H), 2.64-2.59 (m, 2H), 2.59-2.45 (m, 5H), 2.31 (s, 3H), 2.08-1.99 (m, 1H), 1.70-1.59 (m, 6H), 1.49-1.40 (m, 1H), 1.30-1.26 (m, 1H), 1.25-1.18 (m, 3H).

### Example 117—Synthesis of Compound I-133

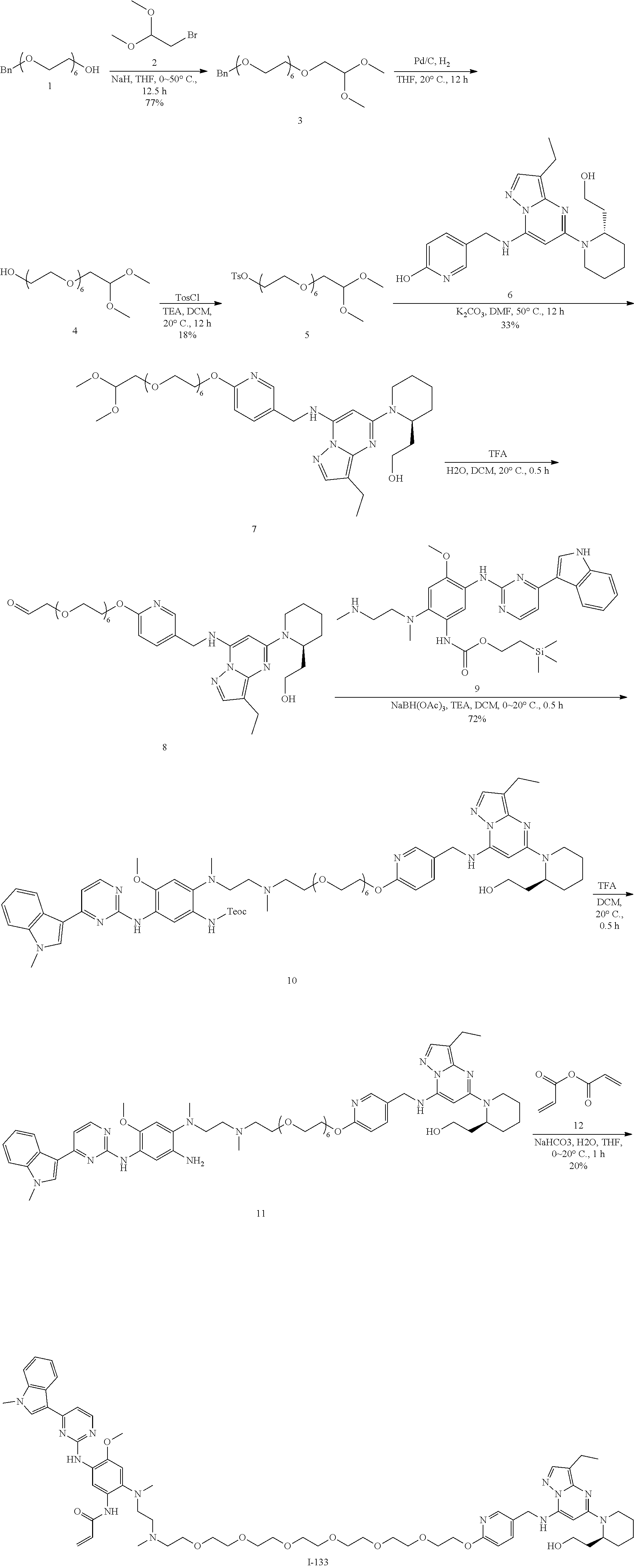

Step 1. To a solution of compound 1 (4.00 g, 10.7 mmol, 1.0 equiv) in THF (30 mL) was added NaH (2.58 g, 64.4 mmol, 60% purity, 6.0 equiv) at 0° C. and stirred for 0.5 h at 20° C. Compound 2 (7.26 g, 43.0 mmol, 5.04 mL, 4.0 equiv) was added at 0° C., and it was stirred at 50° C. for 12 h. This reaction was quenched by aq. NH₄Cl (3 mL), the reaction mixture was concentrated under reduced pressure to give a residue. The residue was purified by column chromatography (SiO₂, Petroleum ether/Ethyl acetate=1/1 to Dichloromethane: Methanol=10/1) to afford compound 2 (3.79 g, 8.23 mmol, 77% yield) as a yellow oil. ¹H NMR: (400 MHz, CDCl₃) δ7.39-7.29 (m, 5H), 4.57 (s, 2H), 4.52 (t, J=5.2 Hz, 1H), 3.68-3.64 (m, 24H), 3.55 (d, J=5.1 Hz, 2H), 3.40 (s, 6H).

Step 2. To a solution of compound 3 (3.79 g, 8.23 mmol, 1.0 equiv) in THF (50 mL) under N2 was added Pd/C (600 mg, 10% purity). The suspension was degassed under vacuum and purged with H₂ several times. The mixture was stirred under H₂ (15 psi) at 20° C. for 12 h. The reaction mixture filtered and concentrated under reduced pressure to give compound 4 (3.5 g, crude) as a yellow oil and used into next step directly. ¹H NMR: (400 MHz, CDCl₃) δ 4.52 (t, J=5.2 Hz, 1H), 3.74-3.71 (m, 2H), 3.67-3.65 (m, 18H), 3.63-3.58 (m, 2H), 3.54 (d, J=5.1 Hz, 2H), 3.48 (s, 3H), 3.40 (s, 6H).

Step 3. To a solution of compound 4 (600 mg, 1.62, 1.0 equiv) in DCM (6 mL) was added TEA (574 mg, 5.67 mmol, 789 μL, 3.5 equiv) and TosCl (772 mg, 4.05 mmol, 2.5 equiv). The mixture was stirred at 20° C. for 12 h. The reaction mixture was concentrated under reduced pressure to give a residue. The residue was purified by prep-HPLC (column: Waters Xbridge 150*25 mm*5 m; mobile phase: [water (NH₄HCO₃)-ACN]; B%: 25%-55%, 10 min) to afford compound 5 (153 mg, 292 μmol, 18% yield) as a yellow oil.

Step 4. To a solution of compound 2 (150 mg, 286 μmol, 1.4 equiv), compound 3 (80.9 mg, 204 μmol, 1.0 equiv) in DMF (3 mL) was added K₂CO₃ (4.68 mg, 613 μmol, 3 equiv). The mixture was stirred at 50° C. for 12 h. The reaction mixture was filtered and concentrated under reduced pressure to give a residue. The residue was purified by prep-HPLC (column: Waters Xbridge 150*25 mm*5 m; mobile phase: [water (NH₄HCO₃)-ACN]; B%: 37%-67%, 10 min) to afford compound 4 (60 mg, 80 μmol, 39% yield) as a yellow oil.

Step 5. To a solution of compound 4 (50 mg, 67 μmol, 1.0 equiv) in DCM (0.5 mL) was added TFA (0.05 mL) and H₂O (0.1 mL). The mixture was stirred at 20° C. for 0.5 h. compound 5 (46.9 mg, crude) was obtained as a brown liquid and used into next step directly.

Step 6. To a solution of compound 8 (46 mg, 65 μmol, 1.0 equiv) and TEA (79.5 mg, 785 μmol, 109 μL, 12 equiv) in DCM (0.5 mL) was added NaBH(OAc)₃ (139 mg, 654 μmol, 10 equiv), compound 6 (69.3 mg, 120 μmol, 1.0 equiv) at 0° C. The mixture was stirred 20° C. for 0.5 h. The reaction mixture was diluted with H₂O (20 mL) and extracted with DCM (10 mL×2). The combined organic layers were dried over Na₂SO₄, filtered and concentrated under reduced pressure to give a residue. The residue was purified by prep-HPLC (column: 3_Phenomenex Luna C18 75*30 mm*3 m; mobile phase: [water (TFA)-ACN]; B%: 32%-62%, 7 min) to afford compound 10 (60 mg, 47 μmol, 72% yield) as a yellow oil.

Step 7. To a solution of compound 10 (56 mg, 44 μmol, 1.0 equiv) in DCM (1 mL) was added TFA (0.5 mL). The mixture was stirred at 20° C. for 0.5 h. The reaction mixture was concentrated under reduced pressure to give a residue to afford compound 11 (55 mg, crude, TFA salt) as a yellow solid and used into next step directly.

Step 8. To a solution of compound 8 (55 mg, 44 μmol, 1.0 equiv, TFA salt) in THF (0.5 mL) and NaHCO₃ (1.08 g, 12.9 mmol, 0.5 mL, 263 equiv) in H₂O (0.5 mL) was added compound 9 (5.6 mg, 44 μmol, 1.0 equiv) at 0° C. The mixture was stirred at 20° C. for 1 h. The reaction mixture was diluted with H₂O (10 mL) and extracted with EA (10 mL×2). The combined organic layers were dried over Na₂SO₄, filtered and concentrated under reduced pressure to give a residue. The residue was purified by prep-HPLC (column: Waters Xbridge 150*25 mm*5 m; mobile phase: [water (NH₄HCO₃)-ACN]; B%: 64%-94%, 8 min) to afford compound I-133 (11 mg, 8 μmol, 20% yield) as a yellow solid. LC-MS: MS (ES⁺): RT=2.252 min, m/z=1173.7 [M+H]+; LCMS method: 25. ¹H NMR: (400 MHz, CD₃OD) 8 9.53 (s, 1H), 8.70 (s, 1H), 8.28 (d, J=5.4 Hz, 1H), 8.17-8.11 (m, 2H), 7.68 (d, J=2.5, 8.5 Hz, 1H), 7.62 (s, 1H), 7.44 (d, J=7.8 Hz, 1H), 7.26-7.17 (m, 3H), 6.98-6.96 (m, 1H), 6.74 (d, J=8.5 Hz, 1H), 6.67 (d, J=10.1, 17.0 Hz, 1H), 6.40 (d, J=1.7, 16.9 Hz, 1H), 5.82-5.76 (m, 1H), 5.46 (s, 1H), 4.79 (d, J=2.1 Hz, 2H), 4.47 (s, 2H), 4.34 (d, J=3.9, 5.4 Hz, 2H), 3.94 (s, 6H), 3.77-3.73 (m, 2H), 3.61-3.57 (m, 2H), 3.55-3.46 (m, 9H), 3.45-3.41 (m, 11H), 3.38-3.35 (m, 1H), 3.01-2.97 (m, 2H), 2.96-2.90 (m, 1H), 2.70 (s, 3H), 2.62 (t, J=5.4 Hz, 2H), 2.58-2.52 (m, 2H), 2.52-2.47 (m, 2H), 2.31 (s, 3H), 2.06-2.00 (m, 1H), 1.72-1.59 (m, 6H), 1.51-1.43 (m, 1H), 1.32-1.27 (m, 1H), 1.22 (t, J=7.6 Hz, 3H).

### Example 118—Synthesis of Compound I-134

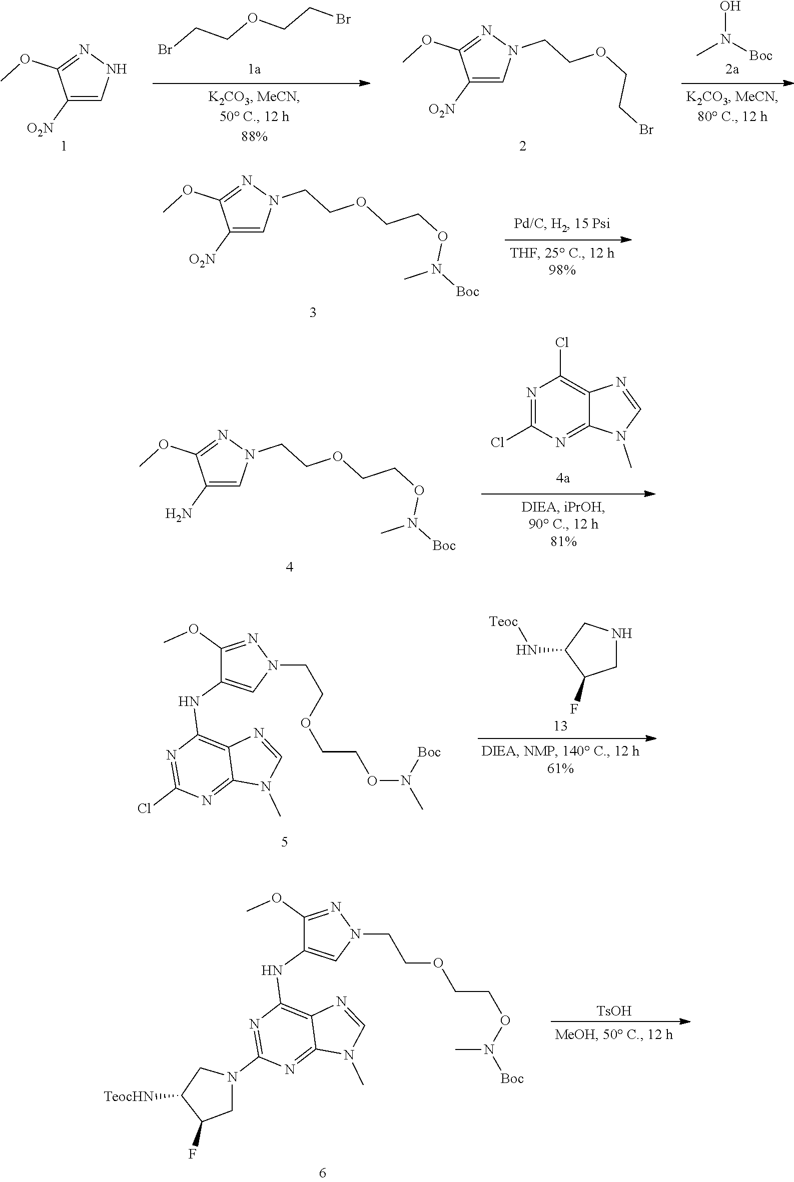

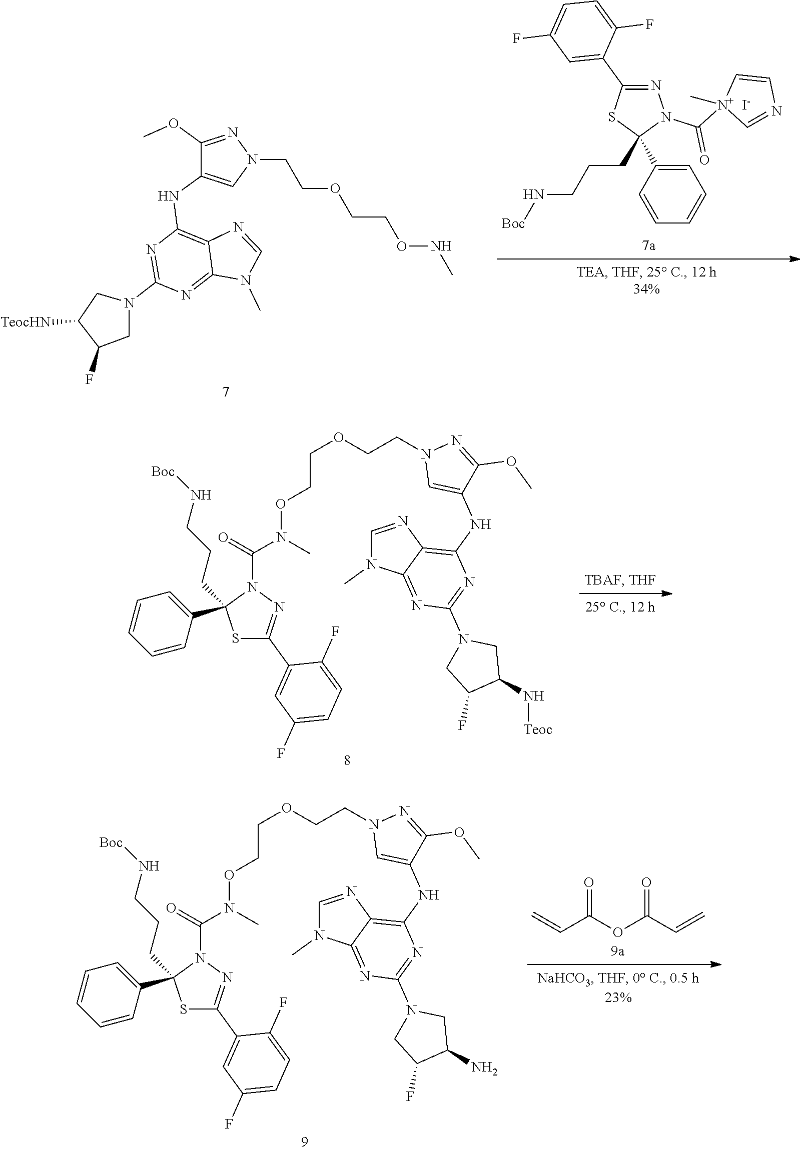

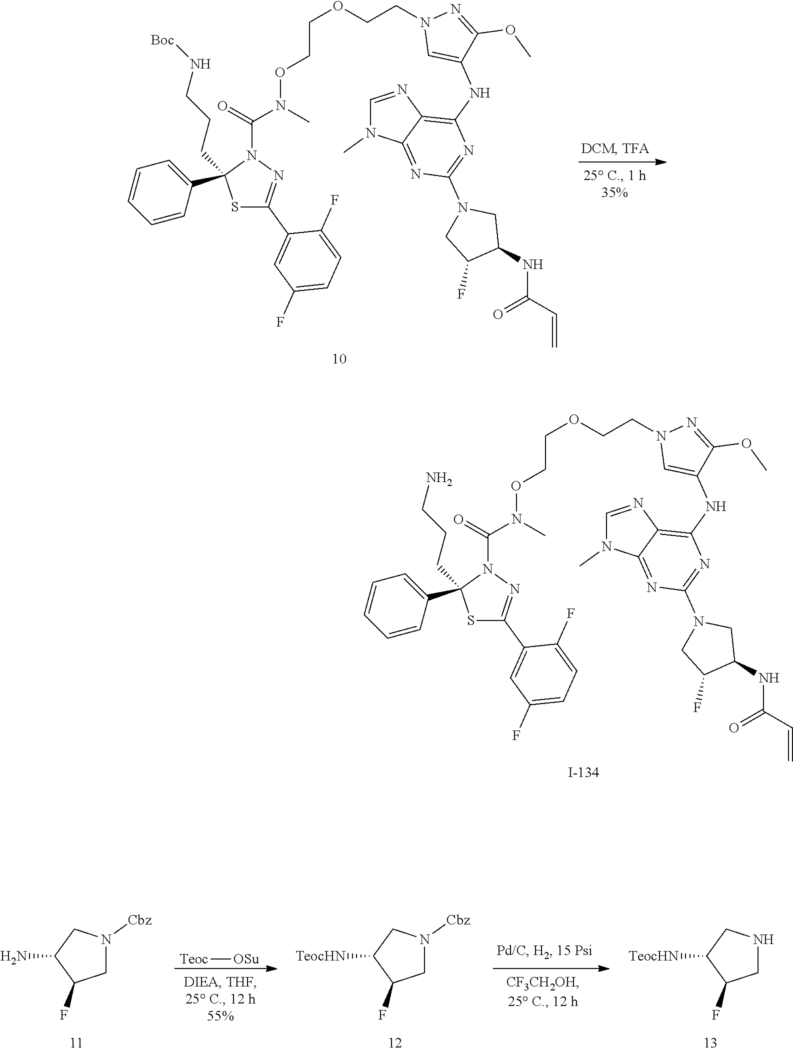

General Information: Compound 11 is a known compound from Organic Process Research and Development, 2019, p. 1970-1978. Compound 7a is a known compound from WO2006/44825.

Step 1. To a solution of 3-methoxy-4-nitro-1H-pyrazole (500 mg, 3.49 mmol, 1.0 equiv) in MeCN (10 mL) was added K₂CO₃ (724 mg, 5.24 mmol, 1.5 equiv) and 1-bromo-2-(2-bromoethoxy)ethane (2.43 g, 10.5 mmol, 3.0 equiv). The mixture was stirred at 50° C. for 12 h and concentrated. The residue was purified by silica column chromatography on silica gel (Petroleum ether: Ethyl acetate from 50/1 to 1/1) to give 1-[2-(2-bromoethoxy)ethyl]-3-methoxy-4-nitro-pyrazole (0.9 g, 88% yield).

Step 2. To a solution of 1-[2-(2-bromoethoxy)ethyl]-3-methoxy-4-nitro-pyrazole (850 mg, 2.89 mmol, 1.0 equiv) in MeCN (10 mL) was added K₂CO₃ (599 mg, 4.34 mmol, 1.5 equiv) and tert-butyl N-hydroxy-N-methyl-carbamate (510 mg, 3.47 mmol, 1.2 equiv). The mixture was stirred at 80° C. for 12 h and concentrated. The residue was purified by silica column chromatography on silica gel (Petroleum ether: Ethyl acetate from 50/1 to 1/1) to give tert-butyl N-[2-[2-(3-methoxy-4-nitro-pyrazol-1-yl)ethoxy]ethoxy]-N-methyl-carbamate (1.1 g, crude). ¹H NMR (400 MHz, DMSO-d₆): δ8.72-8.64 (m, 1H), 4.19 (t, J=5.2 Hz, 2H), 3.93 (s, 3H), 3.86 (m, J=3.6, 5.6 Hz, 2H), 3.79 (t, J=5.2 Hz, 2H), 3.57 (m, J=3.6, 5.6 Hz, 2H), 2.94 (s, 3H), 1.39 (s, 9H).

Step 3. To a solution of tert-butyl N-[2-[2-(3-methoxy-4-nitro-pyrazol-1-yl)ethoxy]ethoxy]-N-methyl-carbamate (1 g, 2.77 mmol, 1.0 equiv) in THF (15 mL) was added Pd/C (200 mg, 10% purity) under N₂. The mixture was stirred under H₂ (15 psi) at 25° C. for 12 h. The mixture was filtered and concentrated to give tert-butyl N-[2-[2-(4-amino-3-methoxy-pyrazol-1-yl)ethoxy]ethoxy]-N-methyl-carbamate (900 mg, 98% yield).

Step 4. To a solution of tert-butyl N-[2-[2-(4-amino-3-methoxy-pyrazol-1-yl)ethoxy]ethoxy]-N-methyl-carbamate (900 mg, 2.72 mmol, 1.0 equiv) in i-PrOH (20 mL) was added DIEA (1.06 g, 8.17 mmol, 3.0 equiv) and 2,6-dichloro-9-methyl-purine (719 mg, 3.54 mmol, 1.3 equiv). The mixture was stirred at 90° C. for 12 h. The mixture was concentrated. The residue was purified by prep-HPLC (column: Waters Xbridge Cis 150*50 mm*10 m; mobile phase: [water (NH₄HCO₃)-ACN]; B%: 30%-60%, 11 min) to give tert-butyl N-[2-[2-[4-[(2-chloro-9-methyl-purin-6-yl)amino]-3-methoxy-pyrazol-1-yl]ethoxy]ethoxy]-N-methyl-carbamate (1.1 g, 81% yield). ¹H NMR (400 MHz, DMSO-d₆): δ9.19 (s, 1H), 8.17 (s, 1H), 7.72 (s, 1H), 4.11 (t, J=5.2 Hz, 2H), 3.94-3.85 (m, 2H), 3.83-3.74 (m, 5H), 3.71 (s, 3H), 3.60-3.55 (m, 2H), 2.97 (s, 3H), 1.39 (s, 9H).

Step 5. To a solution of tert-butyl N-[2-[2-[4-[(2-chloro-9-methyl-purin-6-yl)amino]-3-methoxy-pyrazol-1-yl]ethoxy]ethoxy]-N-methylcarbamate (600 mg, 1.21 mmol, 1.0 equiv) in NMP (6 mL) was added DIEA (468 mg, 3.62 mmol, 3.0 equiv) and 2-trimethylsilylethyl N-[(3R,4R)-4-fluoropyrrolidin-3-yl]carbamate (450 mg, 1.81 mmol, 1.5 equiv). The mixture was stirred at 140° C. for 12 h and concentrated. The residue was purified by prep-HPLC (column: Waters Xbridge C18 150*50 mm*10 μm; mobile phase: [water (NH₄HCO₃)-ACN]; B%: 58%-88%, 11 min) to give tert-butyl N-[2-[2-[4-[[2-[(3R,4R)-3-fluoro-4-(2-trimethylsilylethoxycarbonyl amino)pyrrolidin-1-yl]-9-methyl-purin-6-yl]amino]-3-methoxy-pyrazol-1-yl]ethoxy]ethoxy]-N-methyl-carbamate (520 mg, 61% yield).

Step 6. To a solution of tert-butyl N-[2-[2-[4-[[2-[(3R,4R)-3-fluoro-4-(2-trimethylsilyl ethoxycarbonylamino)pyrrolidin-1-yl]-9-methylpurin-6-yl]amino]-3-methoxy-pyrazol-1-yl]ethoxy]ethoxy]-N-methyl-carbamate (300 mg, 423 μmol, 1.0 equiv) in MeOH (5 mL) was added TsOH.H₂O (201 mg, 1.06 mmol, 2.5 equiv). The mixture was stirred at 50° C. for 12 h. The mixture was concentrated to give 2-trimethylsilylethyl N-[(3R,4R)-4-fluoro-1-[6-[[3-methoxy-1-[2-[2-(methylaminooxy)ethoxy]ethyl]pyrazol-4-yl]amino]-9-methyl-purin-2-yl]pyrrolidin-3-yl]carbamate (300 mg, crude, TsOH salt).

Step 7. To a solution of 2-trimethylsilylethyl N-[(3R,4R)-4-fluoro-1-[6-[[3-methoxy-1-[2-[2-(methylaminooxy)ethoxy]ethyl]pyrazol-4-yl]amino]-9-methyl-purin-2-yl]pyrrolidin-3-yl]carbamate (300 mg, 384 μmol, 1.0 equiv, TsOH salt) in THF (8 mL) was added TEA (117 mg, 1.15 mmol, 3.0 equiv) and tert-butyl N-[3-[(2S)-5-(2,5-difluorophenyl)-3-(1-methylimidazol-1-ium-1-carbonyl)-2-phenyl-1,3,4-thiadiazol-2-yl]propyl]carbamate; iodide (386 mg, 576 μmol, 1.5 equiv). The mixture was stirred at 25° C. for 12 h and concentrated. The residue was purified by prep-HPLC (column: Unisil 3-100 C18 Ultra 150*50 mm*3 m; mobile phase: [water (FA)-ACN]; B%: 65%-85%, 10 min) to give 2-trimethylsilylethyl N-[(3R,4R)-1-[6-[[1-[2-[2-[[(2S)-2-[3-(tert-butoxycarbonylamino)propyl]-5-(2,5-difluorophenyl)-2-phenyl-1,3,4-thiadiazole-3-carbonyl]-methyl-amino]oxyethoxy]ethyl]-3-methoxy-pyrazol-4-yl]amino]-9-methyl-purin-2-yl]-4-fluoro-pyrrolidin-3-yl]carbamate (140 mg, 34% yield). ¹H NMR (400 MHz, DMSO-d₆): δ 7.99 (s, 1H), 7.88-7.75 (m, 3H), 7.54 (d, J=5.2 Hz, 1H), 7.46-7.35 (m, 5H), 7.30-7.24 (m, 1H), 7.22-7.14 (m, 1H), 6.89 (t, J=5.6 Hz, 1H), 5.22-4.97 (m, 1H), 4.22-4.11 (m, 1H), 4.09-3.96 (m, 6H), 3.83-3.66 (m, 8H), 3.63-3.51 (m, 6H), 3.02 (s, 5H), 2.85-2.76 (m, 1H), 1.78 (s, 1H), 1.59-1.19 (m, 11H), 0.90 (t, J=8.0 Hz, 2H), 0.00 (s, 9H).

Step 8. To a solution of 2-trimethylsilylethyl N-[(3R,4R)-1-[6-[[1-[2-[2-[[(2S)-2-[3-(tert-butoxycarbonylamino)propyl]-5-(2,5-difluorophenyl)-2-phenyl-1,3,4-thiadiazole-3-carbonyl]-methyl-amino]oxyethoxy]ethyl]-3-methoxy-pyrazol-4-yl]amino]-9-methyl-purin-2-yl]-4-fluoro-pyrrolidin-3-yl]carbamate (140 mg, 131 μmol, 1.0 equiv) in THF (1.5 mL) was added TBAF (1 M, 1.5 mL, 11.5 equiv). The mixture was stirred at 25° C. for 12 h. The reaction mixture was diluted with water (10 mL) and extracted with ethyl acetate (15 mL*3). The combined organic layers were washed with brine (20 mL), dried over sodium sulfate, filtered and concentrated to give tert-butyl N-[3-[(2S)-3-[2-[2-[4-[[2-[(3R,4R)-3-amino-4-fluoro-pyrrolidin-1-yl]-9-methyl-purin-6-yl]amino]-3-methoxypyrazol-1-yl]ethoxy]ethoxy-methyl-carbamoyl]-5-(2,5-difluorophenyl)-2-phenyl-1,3,4-thiadiazol-2-yl]propyl]carbamate (150 mg, crude).

Step 9. To a solution of tert-butyl N-[3-[(2S)-3-[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy-methyl-carbamoyl]-5-(2,5-difluorophenyl)-2-phenyl-1,3,4-thiadiazol-2-yl]propyl]carbamate (120 mg, 130 μmol, 1.0 equiv) in THF (4 mL) was added NaHCO₃ (1 M, 2.0 mL, 15.4 equiv) and prop-2-enoylprop-2-enoate (20 mg, 156 μmol, 1.2 equiv). The mixture was stirred at 0° C. for 0.5 h. The reaction mixture was diluted with water (15 mL) and extracted with ethyl acetate (20 mL*3). The combined organic layers were washed with brine (20 mL), dried over sodium sulfate, filtered and concentrated. The residue was purified by prep-HPLC (column: Waters Xbridge 150*25 mm* 5 m; mobile phase: [water (NH₄HCO₃)-ACN]; B%: 55%-85%, 9 min) to give tert-butyl N-[3-[(2S)-5-(2,5-difluorophenyl)-3-[2-[2-[4-[[2-[(3R,4R)-3-fluoro-4-(prop-2-enoylamino)pyrrolidin-1-yl]-9-methylpurin-6-yl]amino]-3-methoxy-pyrazol-1-yl]ethoxy]ethoxy-methyl-carbamoyl]-2-phenyl-1,3,4-thiadiazol-2-yl]propyl]carbamate (30 mg, 23% yield).

Step 10. To a solution of benzyl (3R,4R)-3-amino-4-fluoro-pyrrolidine-1-carboxylate (2 g, 5.68 mmol, 1.0 equiv, TFA salt) in THF (50 mL) was added (2,5-dioxopyrrolidin-1-yl) 2-trimethylsilylethyl carbonate (2.94 g, 11.4 mmol, 2.0 equiv) and DIEA (3.67 g, 28.4 mmol, 5.0 equiv). The mixture was stirred at 25° C. for 12 h and concentrated. The residue was purified by prep-HPLC (column: Waters Xbridge C18 150*50 mm*10 m; mobile phase: [water (NH₄HCO₃)-ACN]; B%: 47%-77%, 11 min) to give benzyl (3R,4R)-3-fluoro-4-(2-trimethylsilyl ethoxycarbonylamino)pyrrolidine-1-carboxylate (1.2 g, 55% yield).

Step 11. To a solution of benzyl (3R,4R)-3-fluoro-4-(2-trimethylsilylethoxycarbonylamino)pyrrolidine-1-carboxylate (1.20 g, 3.14 mmol, 1.0 equiv) in CF₃CH₂OH (15 mL) was added Pd/C (0.2 g, 10% purity) under N₂. The mixture was stirred under H₂ (15 psi) at 25° C. for 12 h. The mixture was filtered and concentrated to give 2-trimethylsilylethyl N-[(3R, 4R)-4-fluoropyrrolidin-3-yl]carbamate (790 mg, crude). ¹H NMR (400 MHz, CDCl₃): δ 5.15-4.79 (m, 2H), 4.16 (d, J=8.0 Hz, 3H), 3.54-3.38 (m, 1H), 3.25-2.98 (m, 2H), 2.71 (d, J=9.6 Hz, 1H), 1.74 (s, 1H), 0.99 (s, 2H), 0.04 (s, 9H).

Step 12. To a solution of tert-butyl N-[3-[(2S)-5-(2,5-difluorophenyl)-3-[-[2-[4-[[2-[(3R,4R)-3-fluoro-4-(prop-2-enoylamino)pyrrolidin-1-yl]-9-methyl-purin-6-yl]amino]-3-methoxy-pyrazol-1-yl]ethoxy]ethoxy-methyl-carbamoyl]-2-phenyl-1,3,4-thiadiazol-2-yl]propyl]carbamate (25 mg, 26 μmol, 1.0 equiv) in DCM (1 mL) was added TFA (0.1 mL). The mixture was stirred at 25° C. for 1 h and concentrated. The residue was purified by prep-HPLC (column: Unisil 3-100 C18 Ultra 150*50 mm*3 m; mobile phase: [water(FA)-ACN]; B%: 20%-40%, 10 min) and prep-HPLC (column: 3_Phenomenex Luna C18 75*30 mm*3 m; mobile phase: [water(TFA)-ACN]; B%: 28%-58%, 7 min) to give (2S)-2-(3-aminopropyl)-5-(2,5-difluorophenyl)-N—[2-[2-[4-[[2-[(3R,4R)-3-fluoro-4-(prop-2-enoylamino)pyrrolidin-1-yl]-9-methyl-purin-6-yl]amino]-3-methoxy-pyrazol-1-yl]ethoxy]ethoxy]-N-methyl-2-phenyl-1,3,4-thiadiazole-3-carboxamide (9 mg, 35% yield). ¹H NMR (400 MHz, DMSO-d₆): δ8.49 (d, J=6.4 Hz, 2H), 8.21 (s, 1H), 7.93 (s, 1H), 7.86-7.65 (m, 4H), 7.48-7.36 (m, 5H), 7.33-7.26 (m, 1H), 7.25-7.17 (m, 1H), 6.28-6.08 (m, 2H), 5.69-5.55 (m, 1H), 5.26-5.01 (m, 1H), 4.53-4.41 (m, 1H), 4.11-4.02 (m, 4H), 3.91-3.78 (m, 6H), 3.73-3.67 (m, 6H), 3.56-3.53 (m, 1H), 3.03 (s, 3H), 2.92 (d, J=6.8 Hz, 3H), 2.64-2.58 (m, 2H), 1.98 (s, 1H), 1.60 (m, J=6.8, 11.6 Hz, 1H). LC-MS: MS (ES′): RT=2.298 min, m/z=878.2 [M+H⁺]; LCMS method: 10.

### Example 119—Synthesis of Compound I-135

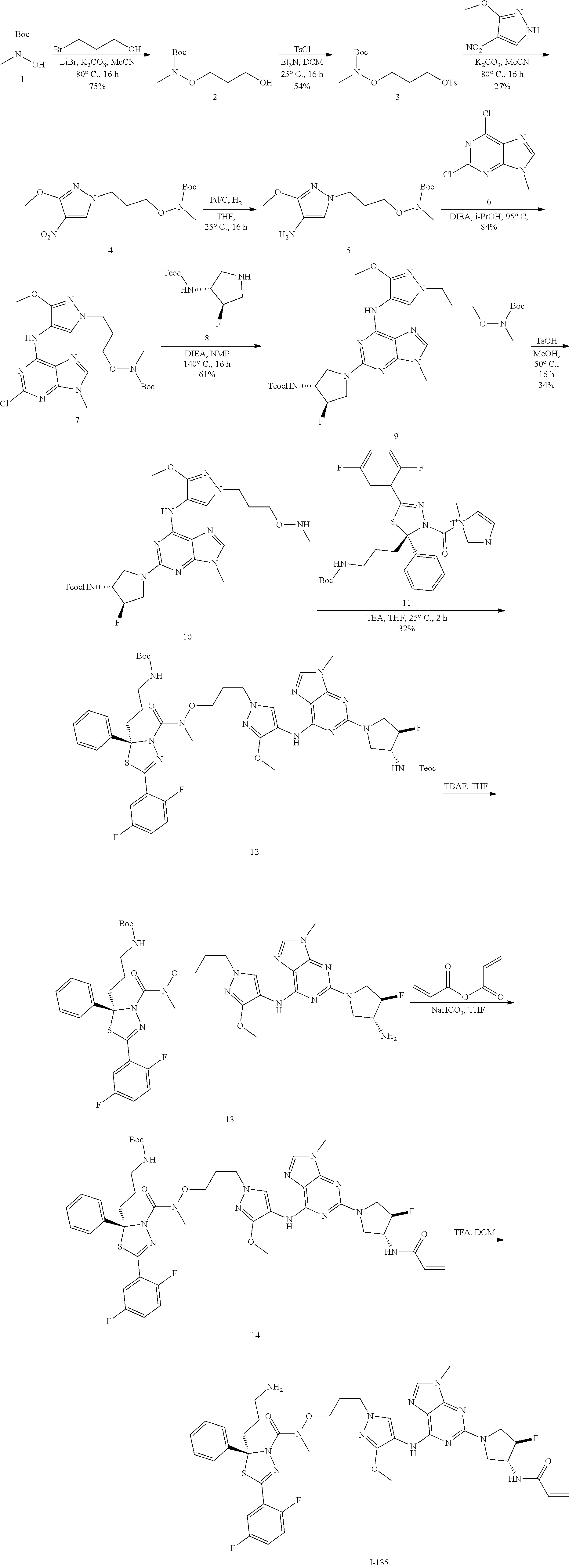

General Information: Compound 11 is a known compound from US2006/247178, 2006, A1.

Step 1. To a solution of tert-butyl N-hydroxy-N-methyl-carbamate (2.0 g, 13.5 mmol, 1.0 equiv) and 3-bromopropan-1-ol (3.78 g, 27.1 mmol, 2.0 equiv) in MeCN (20 mL) was added LiBr (23 mg, 271 μmol, 0.02 equiv) and K₂CO₃ (5.63 g, 40.7 mmol, 3.0 equiv), the mixture was stirred at 80° C. for 16 h. The mixture was filtered and concentrated. The residue was purified by prep-HPLC (column: Waters Xbridge C18 150*50 mm*10 m; mobile phase: [water (NH₄HCO₃)-ACN]; B%: 18%-48%, 11 min) to give tert-butyl N-(3-hydroxypropoxy)-N-methyl-carbamate (2.1 g, 75% yield). ¹H NMR (400 MHz, DMSO-d₆): δ4.51-4.41 (m, 1H), 3.88-3.75 (m, 2H), 3.52-3.43 (m, 2H), 3.04-2.91 (m, 3H), 1.72-1.58 (m, 2H), 1.41 (s, 9H).

Step 2. To a solution of tert-butyl N-(3-hydroxypropoxy)-N-methyl-carbamate (2.10 g, 10.2 mmol, 1.0 equiv) and TEA (3.11 g, 30.6 mmol, 4.2 mL, 3.0 equiv) in DCM (20 mL) was added TosCl (3.90 g, 20.4 mmol, 2.0 equiv) at 0° C., the mixture was stirred at 25° C. for 16 h. The reaction mixture was diluted with water (20 mL) and extracted with EtOAc (30 mL*3). The combined organic layers were washed with brine (50 mL), dried over Na₂SO₄, filtered and concentrated. The residue was purified by column chromatography (SiO₂, Petroleum ether/Ethyl acetate=50/1 to 5/1) to give 3-[tert-butoxycarbonyl(methyl)amino]oxypropyl 4-methylbenzenesulfonate (2.0 g, 54% yield).

Step 3. To a solution of 3-[tert-butoxycarbonyl(methyl)amino]oxypropyl 4-methylbenzenesulfonate (2.00 g, 5.56 mmol, 1.0 equiv) and 3-methoxy-4-nitro-1H-pyrazole (796 mg, 5.56 mmol, 1.0 equiv) in MeCN (20 mL) was added K₂CO₃ (1.54 g, 11.1 mmol, 2.0 equiv), the mixture was stirred at 80° C. for 16 h. The mixture was filtered and concentrated. The residue was purified by column chromatography (SiO₂, Petroleum ether/Ethyl acetate=20/1 to 2/1) to give tert-butyl N-[3-(3-methoxy-4-nitro-pyrazol-1-yl)propoxy]-N-methyl-carbamate (500 mg, 27% yield). ¹H NMR (400 MHz, DMSO-d₆): δ8.74 (s, 1H), 4.18-4.09 (m, 2H), 3.93 (s, 3H), 3.84-3.76 (m, 2H), 3.03-2.99 (m, 3H), 2.09-1.97 (m, 2H), 1.41 (s, 9H).

Step 4. To a solution of tert-butyl N-[3-(3-methoxy-4-nitro-pyrazol-1-yl)propoxy]-N-methyl-carbamate (400 mg, 1.21 mmol, 1.0 equiv) in THF (6 mL) was added Pd/C (80 mg, 10% purity) under N₂. The mixture was stirred under H₂ (15 psi) at 25° C. for 16 h. The mixture was filtered and concentrated to give crude tert-butyl N-[3-(4-amino-3-methoxy-pyrazol-1-yl)propoxy]-N-methyl-carbamate (360 mg) which was used into the next step without further purification.

Step 5. To a solution of tert-butyl N-[3-(4-amino-3-methoxy-pyrazol-1-yl)propoxy]-N-methyl-carbamate (440 mg, 1.46 mmol, 1.0 equiv) and 2,6-dichloro-9-methyl-purine (386 mg, 1.9 mmol, 1.3 equiv) in i-PrOH (6 mL) was added DIEA (946 mg, 7.3 mmol, 5.0 equiv). The mixture was stirred at 90° C. for 16 h. The mixture was concentrated and purified by prep-HPLC (column: Waters Xbridge C18 150*50 mm*10 m; mobile phase: [water (NH₄HCO₃)-ACN]; B%: 33%-63%, 11 min) to give tert-butyl N-[3-[4-[(2-chloro-9-methyl-purin-6-yl)amino]-3-methoxy-pyrazol-1-yl]propoxy]-N-methyl-carbamate (575 mg, 84% yield).

Step 6. To a solution of tert-butyl N-[3-[4-[(2-chloro-9-methyl-purin-6-yl)amino]-3-methoxy-pyrazol-1-yl]propoxy]-N-methyl-carbamate (575 mg, 1.23 mmol, 1.0 equiv) and 2-trimethylsilylethyl N-[(3R,4R)-4-fluoropyrrolidin-3-yl]carbamate (428 mg, 1.72 mmol, 1.4 equiv) in NMP (4 mL) was added DIEA (795 mg, 6.2 mmol, 5.0 equiv). The mixture was stirred at 140° C. for 16 h. The mixture was concentrated and purified by prep-HPLC (column: Waters Xbridge C18 150*50 mm*10 m; mobile phase: [water (NH₄HCO₃)-ACN]; B%: 49%-79%, 11 min) to give tert-butyl N-[3-[4-[[2-[(3R,4R)-3-fluoro-4-(2-trimethylsilylethoxycarbonylamino)pyrrolidin-1-yl]-9-methyl-purin-6-yl]amino]-3-methoxy-pyrazol-1-yl]propoxy]-N-methyl-carbamate (510 mg, 61% yield). ¹H NMR (400 MHz, DMSO-d₆): δ8.07-8.01 (m, 1H), 7.91-7.83 (m, 1H), 7.78 (s, 1H), 7.59-7.48 (m, 1H), 4.21-4.13 (m, 1H), 4.11-4.02 (m, 4H), 3.86-3.69 (m, 8H), 3.64-3.53 (m, 4H), 3.32-3.28 (m, 1H), 3.08-2.93 (m, 3H), 2.05-1.89 (m, 2H), 1.41 (s, 9H), 1.00-0.86 (m, 2H), 0.02 (s, 9H).

Step 7. To a solution of tert-butyl N-[3-[4-[[2-[(3R,4R)-3-fluoro-4-(2-trimethylsilyl ethoxycarbonylamino)pyrrolidin-1-yl]-9-methyl-purin-6-yl]amino]-3-methoxy-pyrazol-1-yl]propoxy]-N-methyl-carbamate (510 mg, 751 μmol, 1.0 equiv) in MeOH (6 mL) was added TsOH (357 mg, 1.88 mmol, 2.5 equiv). The mixture was stirred at 50° C. for 16 h. The mixture was concentrated and purified by prep-HPLC (column: Waters Xbridge C18 150*50 mm* 10 μm; mobile phase: [water (NH₄HCO₃)-ACN]; B%: 37%-67%, 11 min) to give 2-trimethylsilylethyl N-[(3R,4R)-4-fluoro-1-[6-[[3-methoxy-1-[3-(methylaminooxy)propyl]pyrazol -4-yl]amino]-9-methyl-purin-2-yl]pyrrolidin-3-yl]carbamate (150 mg, 34% yield).

Step 8. To a solution of tert-butyl N-[3-[5-(2,5-difluorophenyl)-3-(3-BLAH-3-methyl-imidazole-1-carbonyl)-2-phenyl-1,3,4-thiadiazol-2-yl]propyl]carbamate (278 mg, 415 μmol, 2 equiv) in THF (2 mL) was added TEA (105 mg, 1.04 mmol, 5 equiv) and 2-trimethylsilylethyl N-[(3R,4R)-4-fluoro-1-[6-[[3-methoxy-1-[3-(methylaminooxy)propyl]pyrazol-4-yl]amino]-9-methyl-purin-2-yl]pyrrolidin-3y1]carbamate (120 mg, 207 μmol, 1 equiv). The mixture was stirred at 25° C. for 2 h and concentrated. The residue was purified by prep-HPLC (column: WatersXbridge 150*25 mm*5 m; mobile phase: [water(NH₄HCO₃)-ACN]; B%: 70%-100%, 9 min) to give 2-trimethylsilylethyl N-[(3R,4R)-1-[6-[[1-[3-[[(2S)-2-[3-(tert-butoxycarbonylamino) propyl]-5-(2,5-difluorophenyl)-2-phenyl-1,3,4-thiadiazole-3-carbonyl]-methyl-amino]oxypropyl]-3-methoxy-pyrazol-4-yl]amino]-9-methyl-purin-2-yl]-4-fluoro-pyrrolidin-3-yl]carbamate (70 mg, 32% yield).

Step 9. A solution of tetrabutylammonium; fluoride; trihydrate (1 M, 1.54 mL, 32 equiv) and 2-trimethylsilylethyl N-[(3R,4R)-1-[6-[[1-[3-[[(2S)-2-[3-(tert-butoxycarbonylamino)propyl]-5-(2,5-difluorophenyl)-2-phenyl-1,3,4-thiadiazole-3-carbonyl]-methylamino]oxypropyl]-3-methoxy-pyrazol-4-yl]amino]-9-methyl-purin-2-yl]-4-fluoro-pyrrolidin-3-yl]carbamate (50 mg, 48.16 μmol, 1 equiv) in THF (1 mL) was stirred at 25° C. for 12 h. The mixture was diluted with water (10 mL) and extracted with DCM (30 mL*3). The combined organic layers were washed with brine (30 mL), dried over Na₂SO₄, filtered and concentrated.

Step 10. To a solution of tert-butyl N-[3-[(2S)-3-[3-[4-[[2-[(3R,4R)-3-amino-4-fluoro-pyrrolidin-1-yl]-9-methyl-purin-6-yl]amino]-3-methoxy-pyrazol-1-yl]propoxy-methyl-carbamoyl]-5-(2,5-difluorophenyl)-2-phenyl-1,3,4-thiadiazol-2-yl]propyl]carbamate (43 mg, 48 μmol, 1 equiv) in THF (0.5 mL) was added NaHCO₃ (1 mL), and prop-2-enoyl prop-2-enoate (6 mg, 48 μmol, 1 equiv) was added at 0° C. The mixture was stirred at 25° C. for 0.5 h. The mixture was diluted with water (5 mL) and extracted with DCM (5 mL*3). The combined organic layers were washed with brine (10 mL), dried over Na₂SO₄, filtered and concentrated. The residue was purified by prep-TLC (SiO₂, DCM: MeOH=10:1) to give tert-butyl N-[3-[(2S)-5-(2,5-difluorophenyl)-3-[3-[4-[[2-[(3R,4R)-3-fluoro-4-(prop-2-enoylamino)pyrrolidin-1-yl]-9-methyl-purin-6-yl]amino]-3-methoxy-pyrazol-1-yl]propoxy-methyl-carbamoyl]-2-phenyl-1,3,4-thiadiazol-2-yl]propyl]carbamate (20 mg, 44% yield).

Step 11. To a solution of tert-butyl N-[3-[(2S)-5-(2,5-difluorophenyl)-3-[3-[4-[[2-[(3R,4R)-3-fluoro-4-(prop-2-enoylamino)pyrrolidin-1-yl]-9-methyl-purin-6-yl]amino]-3-methoxy-pyrazol-1-yl]propoxy-methyl-carbamoyl]-2-phenyl-1,3,4-thiadiazol-2-yl]propyl]carbamate (15 mg, 16 μmol, 1 equiv) in DCM (2 mL) was added TFA (0.2 mL). The mixture was stirred at 25° C. for 0.5 h and concentrated. The residue was purified by prep-HPLC (column: 3_Phenomenex Luna C18 75*30 mm*3 m; mobile phase: [water(TFA)-ACN]; B%: 28%-58%, 7 min) to give (2S)-2-(3-aminopropyl)-5-(2,5-difluorophenyl)-N-[3-[4-[[2-[(3R,4R)-3-fluoro-4-(prop-2-enoylamino)pyrrolidin-1-yl]-9-methyl-purin-6-yl]amino]-3-methoxy-pyrazol-1-yl]propoxy]-N-methyl-2-phenyl-1,3,4-thiadiazole-3-carboxamide (8.58 mg, 56% yield). ¹H NMR (400 MHz, MeOD): δ8.27 (d, J=2.4 Hz, 1H), 8.02 (d, J=4.4 Hz, 1H), 7.80-7.75 (m, 1H), 7.50-7.43 (m, 2H), 7.35-7.18 (m, 3H), 7.15-7.05 (m, 2H), 6.27-6.23 (m, 2H), 5.69-5.66 (m, 1H), 5.20-5.05 (m, 1H), 4.56-4.48 (m, 1H), 4.10-4.05 (m, 2H), 4.00-3.90 (m, 6H), 3.85-3.82 (m, 3H), 3.81 (s, 3H), 3.31-3.25 (m, 3H), 3.15-2.99 (m, 3H), 2.70-2.50 (m, 1H), 2.25-2.20 (m, 3H), 1.85-1.65 (m, 1H). LC-MS: MS (ES′): RT=2.310 min, m/z=848.2 [M+H⁺]; LCMS method: 10.

### Example 120—Synthesis of Compound I-136

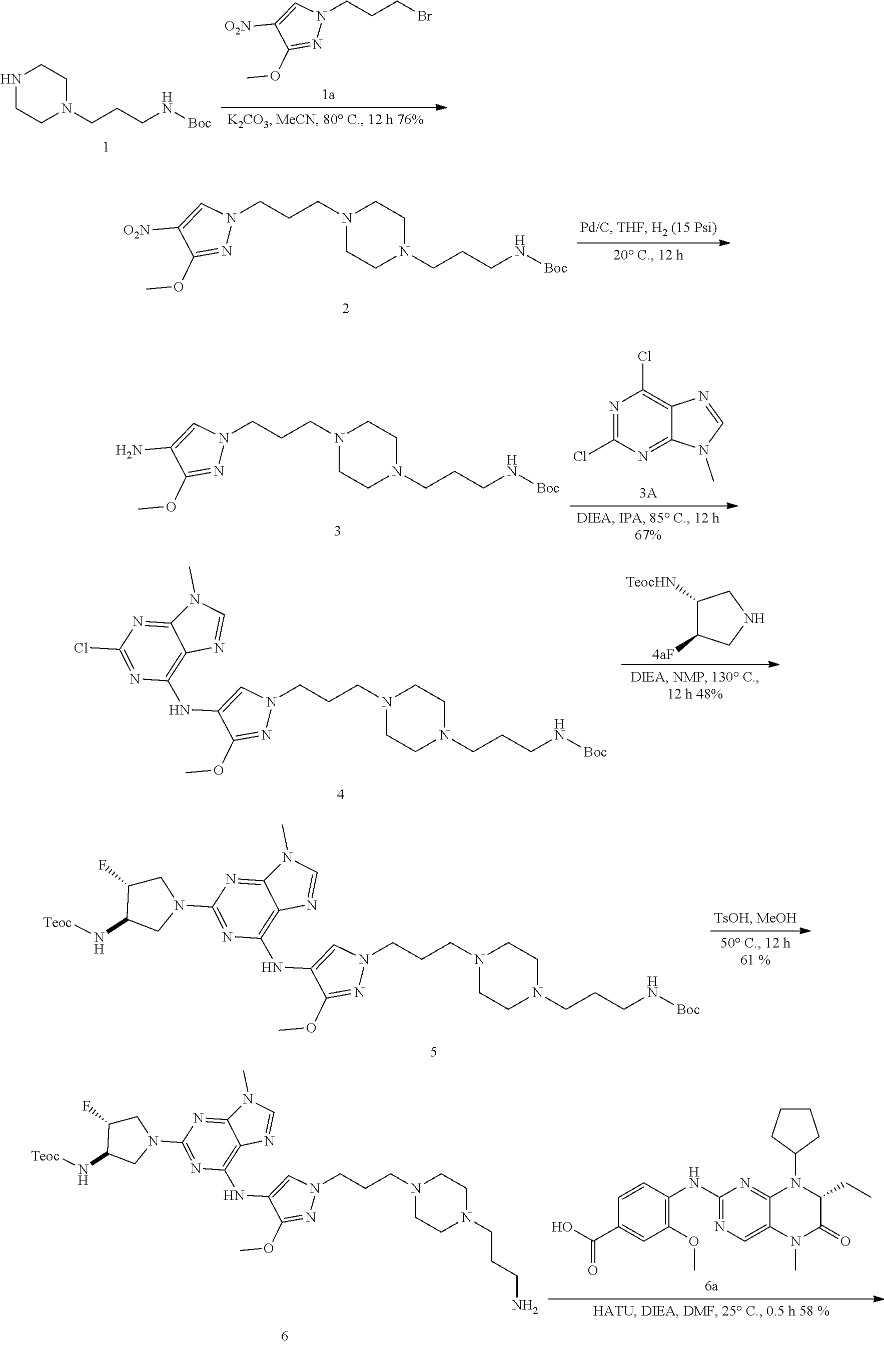

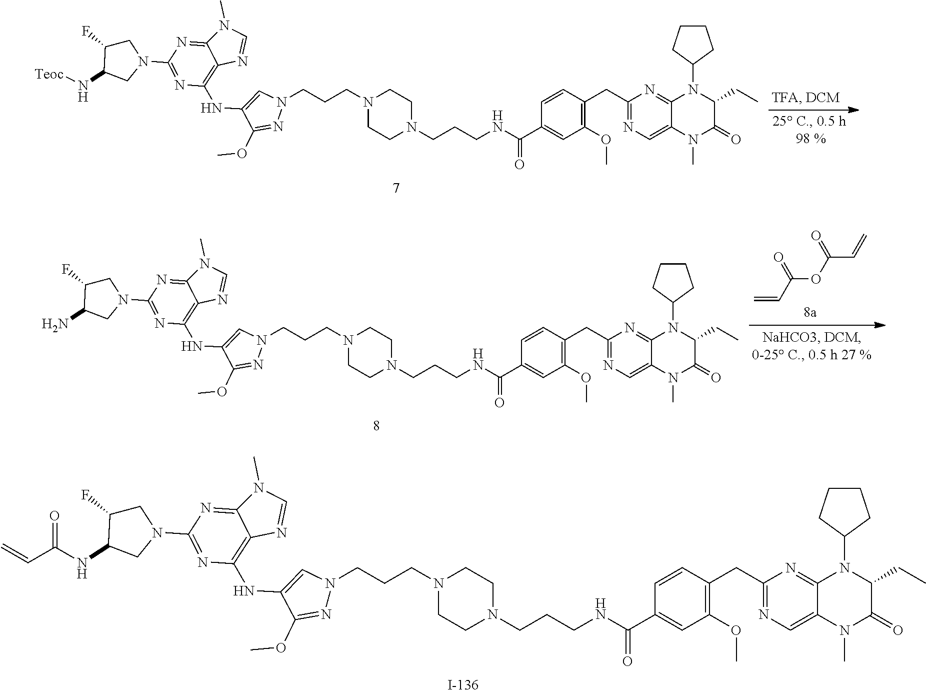

General Information: Synthetic route of compound 8a is a known compound from Journal of the American Chemical Society, 2015, vol. 137, # 8, p. 2836-2839.

Step 1. To a solution of 1-(3-bromopropyl)-3-methoxy-4-nitro-pyrazole (900 mg, 3.41 mmol, 1 equiv) in ACN (15 mL) was added K₂CO₃ (942 mg, 6.82 mmol, 2 equiv) and tert-butyl N-(3-piperazin-1-ylpropyl)carbamate (903 mg, 3.71 mmol, 1.09 equiv). The mixture was stirred at 80° C. for 12 h. The reaction mixture was filtered to remove K₂CO₃ and concentrated to afford crude product. The residue was purified by column chromatography (SiO₂, Dichloromethane: Methanol=10: 1) to give tert-butyl N-[3-[4-[3-(3-methoxy-4-nitro-pyrazol-1-yl)propyl]piperazin-1-yl]propyl]carbamate (1.1 g, 76% yield). ¹H NMR (400 MHz, CDCl₃): δ =7.95 (s, 1H), 4.11-3.92 (m, 7H), 3.15-3.10 (m, 2H), 2.45-2.40 (m, 8H), 2.26 (t, J=6.8 Hz, 2H), 1.99-1.93 (m, 4H), 1.37 (s, 9H).

Step 2. To a solution of tert-butyl N-[3-[4-[3-(3-methoxy-4-nitro-pyrazol-1-yl)propyl]piperazin-1-yl]propyl]carbamate (1.1 g, 2.58 mmol, 1 equiv) in THF (10 mL) was added Pd/C (50 mg, 1.52 mmol, 10% purity) under N₂ atmosphere. The suspension was degassed and purged with H₂ for 3 times. The mixture was stirred under H₂ (15 Psi.) at 20° C. for 12 h. The reaction mixture was filtered and concentrated under reduced pressure to give tert-butyl N-[3-[4-[3-(4-amino-3-methoxy-pyrazol-1-yl)propyl]piperazin-1-yl]propyl]carbamate (1 g, crude) as a yellow oil.

Step 3. To a solution of tert-butyl N-[3-[4-[3-(4-amino-3-methoxy-pyrazol-1-yl)propyl]piperazin-1-yl]propyl]carbamate (1 g, 2.52 mmol, 1 equiv) in IPA (8 mL) was added DIEA (977 mg, 7.57 mmol, 1.32 mL, 3 equiv) and 2,6-dichloro-9-methyl-purine (512 mg, 2.52 mmol, 1 equiv). The mixture was stirred at 85° C. for 12 h and concentrated. The residue was purified by prep-HPLC (column: Waters Xbridge C¹⁸ 150*50 mm*10 μm; mobile phase: [water (NH₄HCO₃)-ACN]; B%: 25%-55%, 10 min) to give tert-butyl N-[3-[4-[3-[4-[(2-chloro-9-methyl-purin-6-yl)amino]-3-methoxy-pyrazol-1-yl]propyl]piperazin-1-yl]propyl]carbamate (947 mg, 67% yield). ¹H NMR (400 MHz, CDCl₃): δ8.07 (s, 2H), 4.08 (t, J=6.4 Hz, 2H), 3.98 (s, 3H), 3.82 (s, 3H), 3.08 (t, J=6.8 Hz, 2H), 2.67-2.33 (m, 12H), 2.07-2.00 (m, 2H), 1.72-1.63 (m, 2H), 1.44 (s, 9H).

Step 4. To a solution of tert-butyl N-[3-[4-[3-[4-[(2-chloro-9-methyl-purin-6-yl)amino]-3-methoxy-pyrazol-1-yl]propyl]piperazin-1-yl]propyl]carbamate (900 mg, 1.60 mmol, 1 equiv) in NMP (5 mL) was added DIEA (1.03 g, 7.99 mmol, 1.39 mL, 5 equiv) and 2-trimethylsilylethyl N-[(3R,4R)-4-fluoropyrrolidin-3-yl]carbamate (793 mg, 3.20 mmol, 2 equiv). The mixture was stirred at 130° C. for 12 h and concentrated. The residue was purified by prep-HPLC (column: Waters Xbridge C18 150*50 mm*10 m; mobile phase: [water (NH₄HCO₃)-ACN]; B%: 44%-74%, 10 min) to give 2-trimethylsilylethyl N-[(3R,4R)-1-[6-[[1-[3-[4-[3-(tert-butoxycarbonylamino)propyl]piperazin-1-yl]propyl]-3-methoxy-pyrazol-4-yl]amino]-9-methyl-purin-2-yl]-4-fluoro-pyrrolidin-3-yl]carbamate (590 mg, 48% yield).

Step 5. To a solution of 2-trimethylsilylethyl N-[(3R,4R)-1-[6-[[1-[3-[4-[3-(tert-butoxycarbonylamino)propyl]piperazin-1-yl]propyl]-3-methoxy-pyrazol-4-yl]amino]-9-methyl-purin-2-yl]-4-fluoro-pyrrolidin-3-yl]carbamate (300 mg, 387 μmol, 1 equiv) in MeOH (5 mL) was added 4-methylbenzenesulfonic acid (199 mg, 1.16 mmol, 3 equiv). The mixture was stirred at 50° C. for 12 h and concentrated. The residue was purified by prep-HPLC (column: Waters Xbridge C18 150*50 mm*10 m; mobile phase: [water (NH₄HCO₃)-ACN]; B%: 42%-72%, 10 min) to give 2-trimethylsilylethyl-N-[(3R,4R)-1-[6-[[1-[3-[4-(3-aminopropyl)piperazin-1-yl]propyl]-3-methoxy-pyrazol-4-yl]amino]-9-methyl-purin-2-yl]-4-fluoro-pyrrolidin-3-yl]carbamate (160 mg, 61% yield). ¹H NMR (400 MHz, CDCl₃): δ=8.01 (s, 1H), 7.69 (s, 1H), 5.20-4.98 (m, 2H), 4.30-4.22 (m, 1H), 4.14-4.10 (m, 2H), 4.01 (t, J=6.4 Hz, 2H), 3.94-3.80 (m, 6H), 3.75-3.64 (m, 4H), 2.73 (t, J=7.2 Hz, 2H), 2.55-2.28 (m, 11H), 2.02-1.93 (m, 2H), 1.71-1.62 (m, 2H), 1.01-0.90 (m, 2H), 0.00 (s, 9H).

Step 6. To a solution of 2-trimethylsilylethyl N-[(3R,4R)-1-[6-[[1-[3-[4-(3-aminopropyl)piperazin-1-yl]propyl]-3-methoxy-pyrazol-4-yl]amino]-9-methyl-purin-2-yl]-4-fluoro-pyrrolidin-3-yl]carbamate (140 mg, 207 μmol, 1 equiv) in DMF (1 mL) was added 4-[[(7R)-8-cyclopentyl-7-ethyl-5-methyl-6-oxo-7H-pteridin-2-yl]amino]-3-methoxy-benzoic acid (88 mg, 207 μmol, 1 equiv), HATU (86 mg, 228 μmol, 1.1 equiv) and DIEA (80 mg, 622 μmol, 3 equiv). The mixture was stirred at 25° C. for 0.5 h and concentrated. The residue was purified by prep-HPLC (column: Waters Xbridge 150*25 mm*5 m; mobile phase: [water (NH₄HCO₃)-ACN]; B%: 58%-88%, 9 min) to give 2-trimethylsilylethyl N-[(3R,4R)-1-[6-[[1-[3-[4-[3-[[4-[[(7R)-8-cyclopentyl-7-ethyl-5-methyl-6-oxo-7H-pteridin-2-yl]amino]-3-methoxy-benzoyl]amino]propyl]piperazin-1-yl]propyl]-3-methoxy-pyrazol-4-yl]amino]-9-methyl-purin-2-yl]-4-fluoro-pyrrolidin-3-yl]carbamate (130 mg, 58% yield).

Step 7. To a solution of 2-trimethylsilylethyl N-[(3R,4R)-1-[6-[[1-[3-[4-[3-[[4-[[(7R)-8-cyclopentyl-7-ethyl-5-methyl-6-oxo-7H-pteridin-2-yl]amino]-3-methoxy-benzoyl]amino]propyl]piperazin-1-yl]propyl]-3-methoxy-pyrazol-4-yl]amino]-9-methyl-purin-2-yl]-4-fluoro-pyrrolidin-3-yl]carbamate (105 mg, 97 μmol, 1 equiv) in DCM (6 mL) was added TFA (3 mL). The mixture was stirred at 25° C. for 0.5 h and concentrated to give crude N-[3-[4-[3-[4-[[2-[(3R,4R)-3-amino-4-fluoro-pyrrolidin-1-yl]-9-methyl-purin-6-yl]amino]-3-methoxy-pyrazol-1-yl]propyl]piperazin-1-yl]propyl]-4-[[(7R)-8-cyclopentyl-7-ethyl-5-methyl-6-oxo-7H-pteridin-2-yl]amino]-3-methoxy-benzamide (100 mg, 98% yield).

Step 8. To a solution of N-[3-[4-[3-[4-[[2-[(3R,4R)-3-amino-4-fluoro-pyrrolidin-1-yl]-9-methyl-purin-6-yl]amino]-3-methoxy-pyrazol-1-yl]propyl]piperazin-1-yl]propyl]-4-[[(7R)-8-cyclopentyl-7-ethyl-5-methyl-6-oxo-7H-pteridin-2-yl]amino]-3-methoxy-benzamide (100 mg, 95 μmol, 1 equiv, TFA salt) in DCM (3 mL) was added NaHCO₃ (2.16 g, 25.71 mmol, 1 mL, 270 equiv) and prop-2-enoyl prop-2-enoate (11 mg, 95 μmol, 1 equiv) at 0° C. The mixture was stirred at 25° C. for 0.5 h and concentrated. The residue was purified by prep-HPLC (column: Unisil 3-100 C18 Ultra 150*50 mm*3 m; mobile phase: [water (FA)-ACN]; B%: 5%-35%, 10 min) to give 4-[[(7R)-8-cyclopentyl-7-ethyl-5-methyl-6-oxo-7H-pteridin-2-yl]amino]-N-[3-[4-[3-[4-[[2-[(3R,4R)-3-fluoro-4-(prop-2-enoylamino)pyrrolidin-1-yl]-9-methyl-purin-6-yl]amino]-3-methoxy-pyrazol-1-yl]propyl]piperazin-1-yl]propyl]-3-methoxy-benzamide (25 mg, 27% yield). ¹H NMR (400 MHz, METHANOL-d4) δ 8.60-8.47 (m, 2H), 8.07 (s, 1H), 7.77 (d, J=10.4 Hz, 2H), 7.53-7.45 (m, 2H), 6.34-6.23 (m, 2H), 5.69 (dd, J=4.0, 8.1 Hz, 1H), 5.27-5.11 (m, 1H), 4.65-4.60 (m, 1H), 4.55-4.50 (m, 1H), 4.29 (dd, J=3.6, 7.6 Hz, 1H), 4.08 (t, J=6.4 Hz, 2H), 4.00 (d, J=13.3 Hz, 1OH), 3.73 (s, 3H), 3.46 (t, J=6.6 Hz, 2H), 2.90-2.41 (m, 12H), 2.27-1.63 (m, 16H), 0.87 (t, J=7.6 Hz, 3H). LC-MS: MS (ES′): RT=1.954 min, m/z=992.4 [M+H⁺]; LCMS method: 10.

### Example 121—Synthesis of Compound I-137

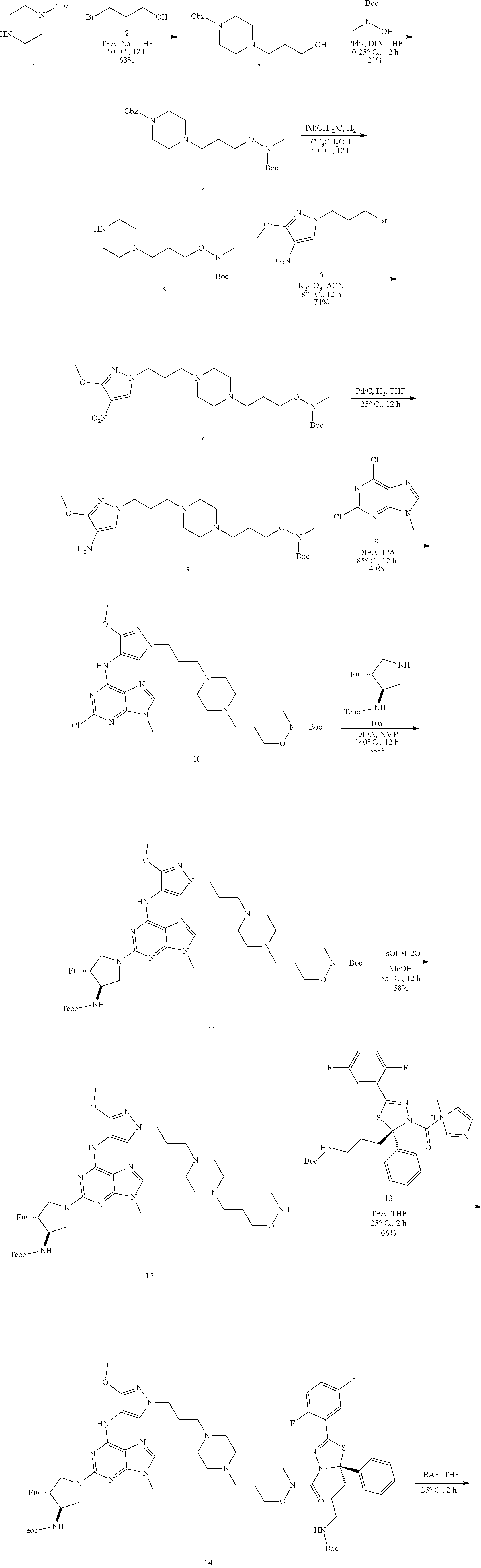

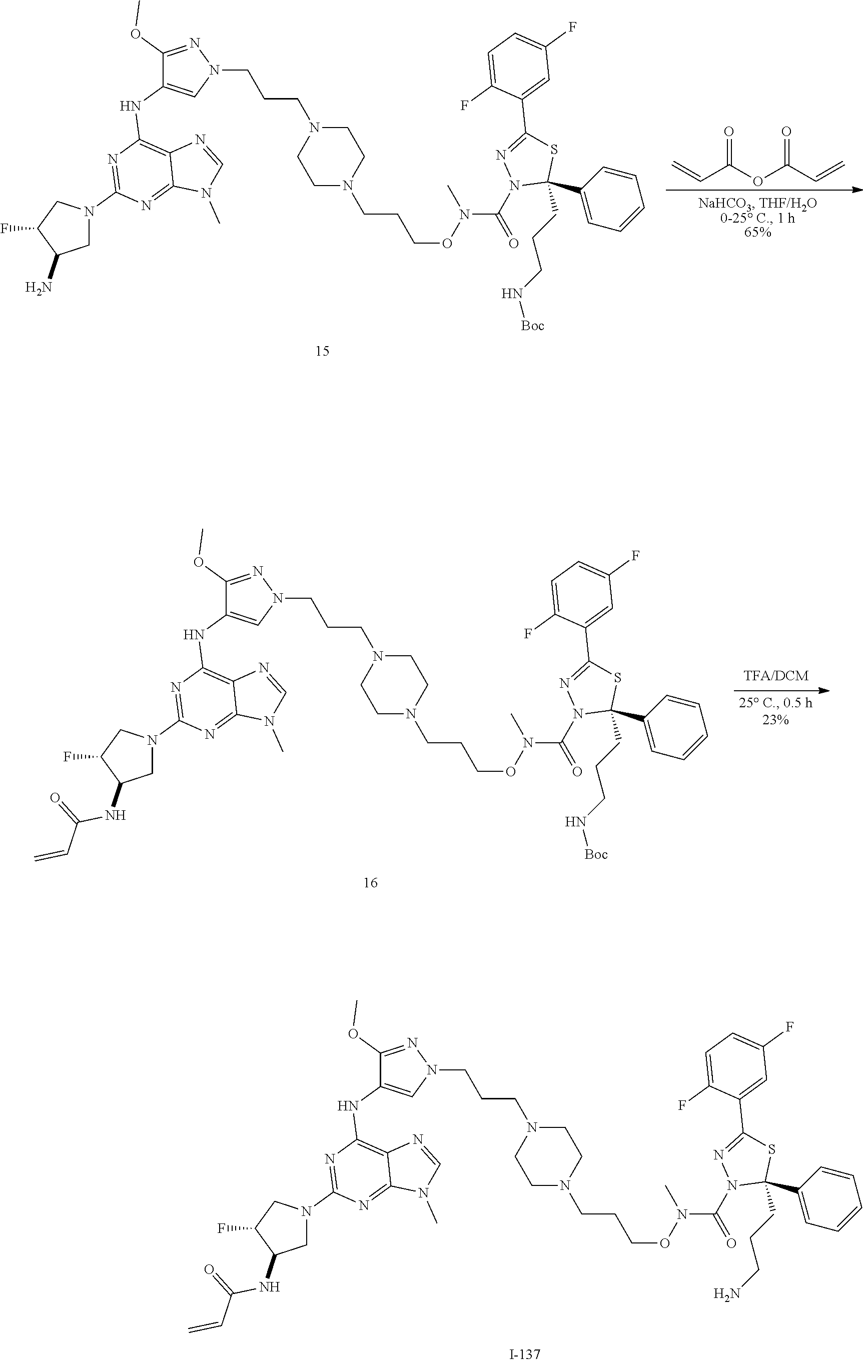

Step 1. The solution of benzyl piperazine-1-carboxylate (10 g, 45 mmol, 8.8 mL, 1.0 equiv) and 3-bromopropan-1-ol (6.3 g, 45.4 mmol, 4.1 mL, 1.0 equiv) and TEA (13.8 g, 136 mmol, 19 mL, 3.0 equiv) and NaI (0.68 g, 4.5 mmol, 0.1 equiv) in THF (200 mL) was stirred at 50° C. for 12 h. The mixture was concentrated under reduced pressure to get the residue, which was purified by prep-HPLC (column: Welch Ultimate XB—CN 250*70*10 μm; mobile phase: [Heptane-EtOH]; B%: 1%-40%, 15 min and column: Kromasil Eternity XT 250*80 mm*10 μm; mobile phase: [water (ammonia hydroxide v/v)-ACN]; B%: 20%-50%, 20 min) to give benzyl 4-(3-hydroxypropyl)piperazine-1-carboxylate (8 g, 63% yield) as a yellow solid. ¹H NMR (400 MHz, CDCl₃): δ7.39-7.21 (m, 5H), 5.16-5.00 (m, 2H), 3.80-3.70 (m, 2H), 3.53-3.37 (m, 4H), 2.62-2.31 (m, 6H), 1.77-1.62 (m, 2H).

Step 2. To the solution of PPh₃ (7.46 g, 28.5 mmol, 1.1 equiv) in THF (90 mL) was added DIAD (5.49 g, 27.2 mmol, 5.28 mL, 1.1 equiv) at 0° C., the solution of benzyl 4-(3-hydroxypropyl)piperazine-1-carboxylate (7.2 g, 25.9 mmol, 1.0 equiv) and tert-butyl N-hydroxy-N-methyl-carbamate (3.81 g, 25.9 mmol, 1.0 equiv) in THF (36 mL) was added to the mixture, then the mixture was stirred at 25° C. for 12 h. The solution was concentrated under reduced pressure to residue, which was purified by prep-HPLC (column: Kromasil Eternity XT 250*80 mm*10 m; mobile phase: [water (NH₄HCO₃)-ACN]; B%: 45%-75%, 20 min) to give benzyl 4-[3-[tert-butoxycarbonyl(methyl)amino]oxypropyl]piperazine-1-carboxylate (2.3 g, 21% yield) as light yellow oil. ¹H NMR (400 MHz, CDCl₃): δ7.47-7.29 (m, 5H), 5.15 (s, 2H), 3.97-3.85 (m, 2H), 3.67-3.46 (m, 4H), 3.11 (s, 3H), 2.58-2.33 (m, 6H), 1.89-1.76 (m, 2H), 1.50 (s, 9H)

Step 3. To a solution of benzyl 4-[3-[tert-butoxycarbonyl(methyl)amino]oxypropyl]piperazine-1-carboxylate (2.3 g, 5.6 mmol, 1.0 equiv) in CF₃CH₂OH (20 mL) was added Pd(OH)₂/C (0.5 g, 10% purity) under N₂. The suspension was degassed under vacuum and purged with H₂ several times. The mixture was stirred under H₂ (15 psi) at 25° C. for 12 h. The mixture was filtered and the filtrate was concentrated under reduced pressure to give tert-butyl N-methyl-N-(3-piperazin-1-ylpropoxy)carbamate (1.54 g, crude) as light yellow oil.

Step 4. The mixture of tert-butyl N-methyl-N-(3-piperazin-1-ylpropoxy)carbamate (1.5 g, 5.6 mmol, 1.0 equiv) and 1-(3-bromopropyl)-3-methoxy-4-nitro-pyrazole (1.5 g, 5.6 mmol, 1.0 equiv) and K₂CO₃ (2.3 g, 17 mmol, 3.0 equiv) in ACN (20 mL) was stirred at 80° C. for 12 h. The mixture was filtered and the filtrate was concentrated to get the residue, which was purified by column chromatography (SiO₂, Petroleum ether/Ethyl acetate=5/1 to Dichloromethane: Methanol=20:1) to give tert-butyl N-[3-[4-[3-(3-methoxy-4-nitro-pyrazol-1-yl)propyl]piperazin-1-yl]propoxy]-N-methyl-carbamate (1.9 g, 74% yield). ¹H NMR (400 MHz, CDCl₃): δ7.95 (s, 1H), 4.10-3.74 (m, 6H), 3.50-3.35 (m, 4H), 3.02 (s, 3H), 2.78-2.44 (m, 7H), 2.39-2.23 (m, 2H), 2.07-1.78 (m, 4H), 1.47-1.27 (m, 9H).

Step 5. To a solution of tert-butyl N-[3-[4-[3-(3-methoxy-4-nitro-pyrazol-1-yl)propyl]piperazin-1-yl]propoxy]-N-methyl-carbamate (2 g, 4.4 mmol, 1.0 equiv) in THF (20 mL) was added Pd/C (0.5 g, 10% purity) under N₂. The mixture was stirred under H₂ (15 psi) at 25° C. for 12 h. The mixture was filtered and concentrated to give tert-butyl N-[3-[4-[3-(4-amino-3-methoxy-pyrazol-1-yl)propyl]piperazin-1-yl]propoxy]-N-methyl-carbamate (1.6 g, crude).

Step 6. The mixture of tert-butyl N-[3-[4-[3-(4-amino-3-methoxy-pyrazol-1-yl) propyl]piperazin-1-yl]propoxy]-N-methyl-carbamate (1.6 g, 3.8 mmol, 1 equiv) and 2,6-dichloro-9-methyl-purine (761 mg, 3.75 mmol, 1.0 equiv) and DIEA (1.45 g, 11 mmol, 3.0 equiv) in IPA (20 mL) was stirred at 85° C. for 12 h. The solution was concentrated under reduced pressure to get the residue, which was purified by column chromatography (SiO₂, Petroleum ether/Ethyl acetate=1/1 to 0/1) to give tert-butyl N-[3-[4-[3-[4-[(2-chloro-9-methyl-purin-6-yl)amino]-3-methoxy-pyrazol-1-yl]propyl]piperazin-1-yl]propoxy]-N-methyl-carbamate (900 mg, 40% yield) as brown oil. ¹H NMR (400 MHz, CDCl₃): δ8.08-7.89 (m, 1H), 7.79-7.67 (m, 1H), 7.53-7.35 (m, 1H), 4.11-3.98 (m, 2H), 3.92-3.80 (m, 4H), 3.78-3.71 (m, 3H), 3.66-3.53 (m, 1H), 3.46-2.70 (m, 10H), 1.55-1.46 (m, 6H), 1.45-1.33 (m, 10H), 1.19 (t, J=7.2 Hz, 2H).

Step 7. The solution of tert-butyl N-[3-[4-[3-[4-[(2-chloro-9-methyl-purin-6-yl)amino]-3-methoxy-pyrazol-1-yl]propyl]piperazin-1-yl]propoxy]-N-methyl-carbamate (600 mg, 1.0 μmmol, 1.0 equiv) and 2-trimethylsilylethyl N-[(3R,4R)-4-fluoropyrrolidin-3-yl]carbamate (377 mg, 1.5 mmol, 1.5 equiv) and DIEA (523 mg, 4.0 mmol, 705 μL, 4.0 equiv) in NMP (6 mL) was stirred at 140° C. for 12 h. The mixture was filtered to get the filtrate, which was purified by prep-HPLC (column: Waters Xbridge C18 150*50 mm*10 μm; mobile phase: [water (NH₄HCO₃)-ACN]; B%: 48%-78%, 10 min) to give tert-butyl N-[3-[4-[3-[4-[[2-[(3R,4R)-3-fluoro-4-(2-trimethylsilyl ethoxycarbonylamino)pyrrolidin-1-yl]-9-methyl-purin-6-yl]amino]-3-methoxy-pyrazol-1-yl]propyl]piperazin-1-yl]propoxy]-N-methyl-carbamate (270 mg, 33% yield).

Step 8. The solution of tert-butyl N-[3-[4-[3-[4-[[2-[(3R,4R)-3-fluoro-4-(2-tri methylsilylethoxycarbonylamino)pyrrolidin-1-yl]-9-methyl-purin-6-yl]amino]-3-methoxy-pyrazol-1-yl]propyl]piperazin-1-yl]propoxy]-N-methyl-carbamate (270 mg, 335 μmol, 1.0 equiv) and TsOH.H₂O (159 mg, 838 μmol, 2.5 equiv) in MeOH (2.5 mL) was stirred at 50° C. for 12 h. The solution was concentrated and purified by prep-HPLC (column: Phenomenex C18 75*30 mm*3 m; mobile phase: [water (FA)-ACN]; B%: 15%-45%, 7 min) to give 2-trimethylsilylethyl N-[(3R,4R)-4-fluoro-1-[6-[[3-methoxy-1-[3-[4-[3-(methylaminooxy)propyl]piperazin-1-yl]propyl]pyrazol-4-yl]amino]-9-methyl-purin-2-yl]pyrrolidin-3-yl]carbamate (170 mg, 58% yield).

Step 9. The solution of 2-trimethylsilylethyl N-[(3R,4R)-4-fluoro-1-[6-[[3-methoxy-1-[3-[4-[3-(methylaminooxy)propyl]piperazin-1-yl]propyl]pyrazol-4-yl]amino]-9-methyl-purin-2-yl]pyrrolidin-3-yl]carbamate (170 mg, 194 μmol, 1.0 equiv, TsOH salt) and tert-butyl N-[3-[(2S)-5-(2,5-difluorophenyl)-3-(1-methylimidazol-1-ium-1-carbonyl)-2-phenyl-1,3,4-thiadiazol-2-yl]propyl]carbamate; iodide (195 mg, 291 μmol, 1.5 equiv) and TEA (87 mg, 862 μmol, 4.5 equiv) in THF (4 mL) was stirred at 25° C. for 2 h. The solution was concentrated and purified by prep-TLC (SiO₂, Dichloromethane: Methanol=10:1) to give 2-trimethylsilylethyl N-[(3R,4R)-1-[6-[[1-[3-[4-[3-[[(2S)-2-[3-(tert-butoxycarbonylamino)propyl]-5-(2,5-difluorophenyl)-2-phenyl-1,3,4-thiadiazole-3-carbonyl]-methyl-amino]oxypropyl]piperazin-1-yl]propyl]-3-methoxy-pyrazol-4-yl]amino]-9-methyl-purin-2-yl]-4-fluoro-pyrrolidin-3-yl]carbamate (150 mg, 66% yield).

Step 10. The solution of 2-trimethylsilylethyl N-[(3R,4R)-1-[6-[[1-[3-[4-[3-[[(2S)-2-[3-(tert-butoxycarbonylamino)propyl]-5-(2,5-difluorophenyl)-2-phenyl-1,3,4-thiadiazole-3-carbonyl]-methyl-amino]oxypropyl]piperazin-1-yl]propyl]-3-methoxy-pyrazol-4-yl]amino]-9-methyl-purin-2-yl]-4-fluoro-pyrrolidin-3-yl]carbamate (150 mg, 129 μmol, 1.0 equiv) in TBAF (1 M, 1 mL, 7.8 equiv) and THF (1 mL) was stirred at 25° C. for 2 h. The solution was used for next step directly.

Step 11. To the mixture of tert-butyl N-[3-[(2S)-3-[3-[4-[3-[4-[[2-[(3R,4R)-3-amino-4-fluoro-pyrrolidin-1-yl]-9-methyl-purin-6-yl]amino]-3-methoxy-pyrazol-1-yl]propyl]piperazin-1-yl]propoxy-methyl-carbamoyl]-5-(2,5-difluorophenyl)-2-phenyl-1,3,4-thiadiazol-2-yl]propyl]carbamate (131 mg, 128 μmol, 1.0 equiv) and NaHCO₃ (4.3 g, 51.4 mmol, 400 equiv) in THF (2 mL) was added prop-2-enoyl prop-2-enoate (81 mg, 642 μmol, 5.0 equiv) at 0° C., then the solution was stirred at 25° C. for 1 h. The solution was extracted with EA (10 mL*3), the combined organic layer was concentrated to get the residue, which was purified by prep-TLC (SiO₂, Dichloromethane: Methanol=10:1) to give tert-butyl N-[3-[(2S)-5-(2,5-difluorophenyl)-3-[3-[4-[3-[4-[[2-[(3R,4R)-3-fluoro-4-(prop-2-enoylamino)pyrrolidin-1-yl]-9-methyl-purin-6-yl]amino]-3-methoxy-pyrazol-1-yl]propyl]piperazin-1-yl]propoxy-methyl-carbamoyl]-2-phenyl-1,3,4-thiadiazol-2-yl]propyl]carbamate (90 mg, 65% yield).

Step 12. The solution of tert-butyl N-[3-[(2S)-5-(2,5-difluorophenyl)-3-[3-[4-[3-[4-[[2-[(3R,4R)-3-fluoro-4-(prop-2-enoylamino)pyrrolidin-1-yl]-9-methyl-purin-6-yl]amino]-3-methoxy-pyrazol-1-yl]propyl]piperazin-1-yl]propoxy-methyl-carbamoyl]-2-phenyl-1,3,4-thiadiazol-2-yl]propyl]carbamate (45 mg, 42 μmol, 1.0 equiv) in TFA (0.1 mL) and DCM (1 mL) was stirred at 25° C. for 0.5 h. The solution was concentrated to get the residue, which was purified by prep-HPLC (column: Phenomenex Luna C18 150*25 mm*10 m; mobile phase: [water(FA)-ACN]; B%: 10%-40%, 10 min) to give (2S)-2-(3-aminopropyl)-5-(2,5-difluorophenyl) —N-[3-[4-[3-[4-[[2-[(3R,4R)-3-fluoro-4-(prop-2-enoylamino)pyrrolidin-1-yl]-9-methyl-purin-6-yl]amino]-3-methoxy-pyrazol-1-yl]propyl]piperazin-1-yl]propoxy]-N-methyl-2-phenyl-1,3,4-thiadiazole-3-carboxamide (9.22 mg, 23% yield). ¹H NMR (400 MHz, MeOD): δ 8.50 (s, 2H), 8.10 (s, 1H), 7.92-7.83 (m, 1H), 7.78 (s, 1H), 7.59-7.51 (m, 2H), 7.43-7.37 (m, 2H), 7.35-7.28 (m, 1H), 7.25-7.10 (m, 2H), 6.34-6.24 (m, 2H), 5.74-5.66 (m, 1H), 5.30-5.11 (m, 1H), 4.23-4.15 (m, 1H), 4.13-3.84 (m, 1OH), 3.74 (s, 3H), 3.37 (s, 3H), 3.19-3.06 (m, 4H), 2.95-2.53 (m, 8H), 2.39-2.12 (m, 5H), 2.08-1.73 (m, 6H). LC-MS: MS (ES⁺): RT=2.131 min, m/z=974.3 [M+H⁺]; LCMS method: 10.

### Example 122—Synthesis of Compound I-138

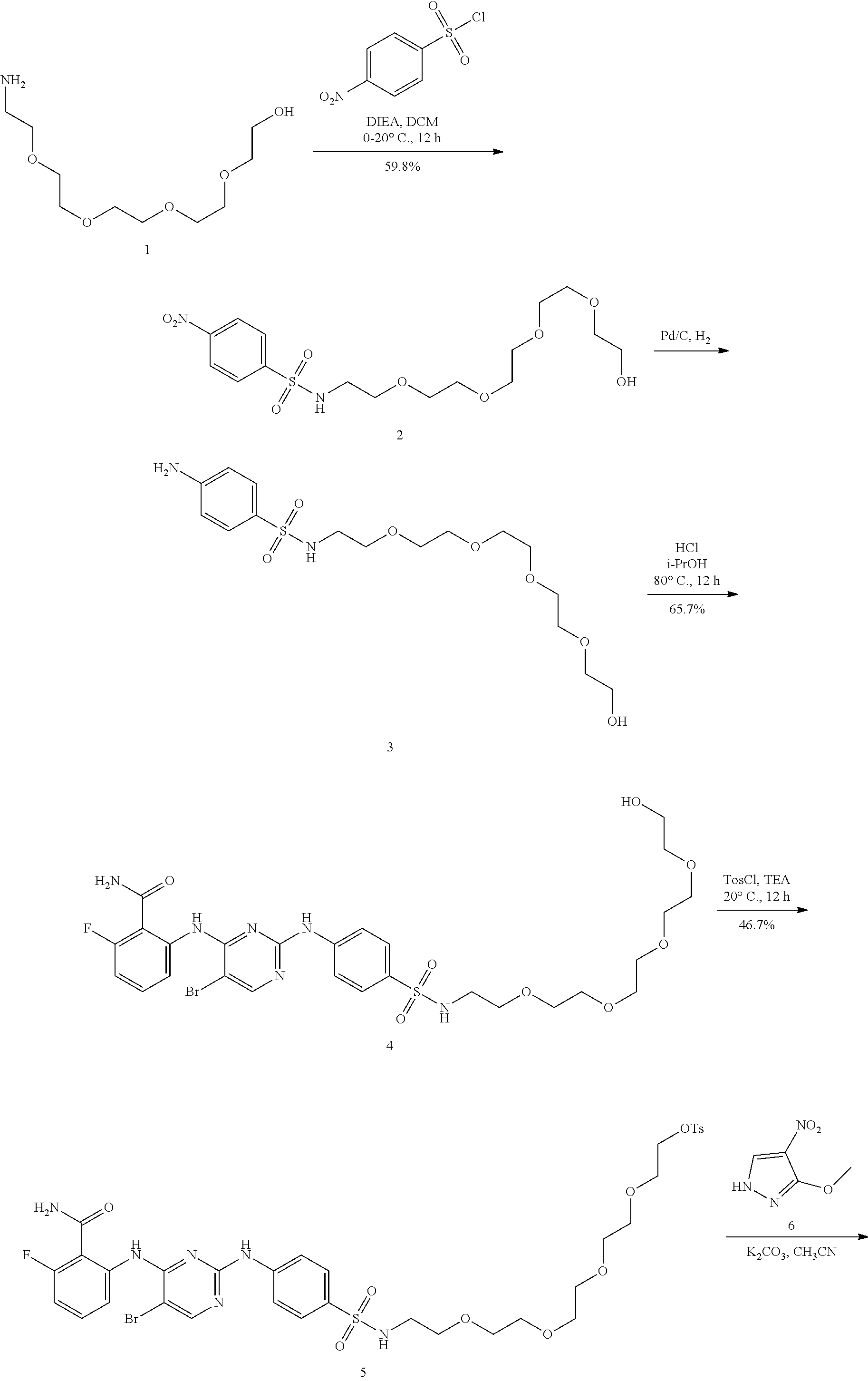

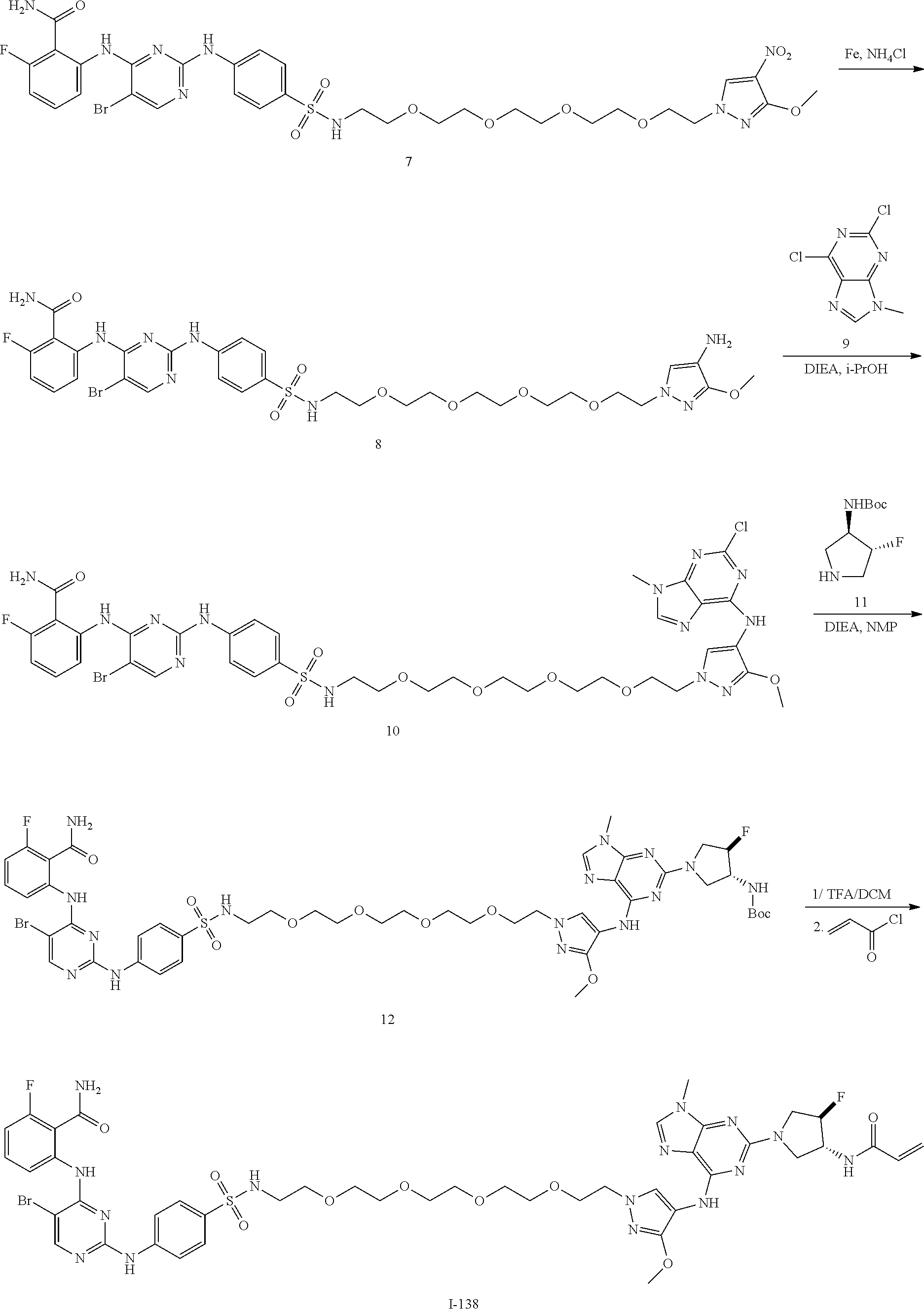

Step 1. To a solution of 2-[2-[2-[2-(2-aminoethoxy)ethoxy]ethoxy]ethoxy]ethanol (2 g, 8.43 mmol, 1 equiv), Et₃N (1.28 g, 12.64 mmol, 1.76 mL, 1.5 equiv) in DCM (30 mL) was added 4-nitrobenzenesulfonyl chloride (2.05 g, 9.27 mmol, 1.1 equiv) at 0° C. The mixture was stirred at 20° C. for 2 hours. TLC (Petroleum ether: Ethyl acetate=1:1) showed that there were two one new spots formed. The mixture was diluted with DCM (50 mL) and washed by water (50 mL), brine (50 mL), the organic layer was dried over Na₂SO₄, concentrated by vacuo. The residue was purified by column chromatography (SiO₂, Petroleum ether: Ethyl acetate=1:0-1:1-0:1). The product N-[2-[2-[2-[2-(2-hydroxyethoxy)ethoxy]ethoxy]ethoxy]ethyl]-4-nitro-benzenesulfon amide (2.1 g, 4.97 mmol, 58.98% yield) was obtained as a yellow oil, and its structure was confirmed by HNMR. ¹H NMR (400 MHz, CDCl₃) δ8.40-8.31 (m, 2H), 8.18-8.09 (m, 2H), 7.44 (brs, 1H), 3.82-3.77 (m, 2H), 3.73-3.63 (m, 11H), 3.61-3.57 (m, 2H), 3.54-3.49 (m, 2H), 3.49-3.44 (m, 2H), 3.23-3.15 (m, 2H).

Step 2. To a solution of Pd/C (200 mg, 497.10 μmol, 10% purity, 0.1 equiv) in MeOH (30 mL) was added N-[2-[2-[2-[2-(2-hydroxyethoxy)ethoxy]ethoxy]ethoxy]ethyl]-4-nitro-benzenesulfonamide (2.1 g, 4.97 mmol, 1 equiv) at 20° C. under N₂. The mixture was degassed and purged with H₂ for 3 times. The mixture was stirred at 20° C. for 12 hours under H₂ (15 Psi). TLC (Dichloromethane: Methanol=10:1) showed that there was one new spot formed. The mixture was filtered, and the filtrate was concentrated by vacuo. The residue was used for the next step without the further purification. The product 4-amino-N—[2-[2-[2-[2-(2-hydroxyethoxy) ethoxy]ethoxy]ethoxy]ethyl]benzenesulfonamide (2 g, crude) was obtained as a yellow oil, and its structure was confirmed by HNMR. ¹H NMR (400 MHz, CDCl₃) δ7.71-7.61 (m, 2H), 6.76-6.62 (m, 2H), 6.14-5.99 (m, 1H), 4.13 (brs, 2H), 3.79-3.74 (m, 2H), 3.71-3.66 (m, 6H), 3.66-3.61 (m, 5H), 3.57-3.45 (m, 5H), 3.32-3.15 (m, 1H), 3.14-3.06 (m, 2H).

Step 3. To a solution of 2-[(5-bromo-2-chloro-pyrimidin-4-yl)amino]-6-fluoro-benzamide (1.50 g, 4.34 mmol, 1 equiv), 4-amino-N-[2-[2-[2-[2-(2-hydroxyethoxy)ethoxy]ethoxy]ethoxy]ethyl]benzenesulfonamide (2.0 g, 5.10 mmol, 1.17 equiv) in i-PrOH (30 mL) was added HCl (12 M, 360 μL, 1 equiv) at 20° C. The mixture was stirred at 80° C. for 12 hours. LCMS and HPLC showed that there was one main peak with the desired Ms detected. The mixture was concentrated by vauco to give a residue. The residue was purified by column chromatography (column: Welch Ultimate XB—CN 250*70*10 m; mobile phase: [Hexane-EtOH (0.1% NH₃.H₂O]; B%: 45%-85%, 15 min). The product 2-[[5-bromo-2-[4-[2-[2-[2-[2-(2-hydroxyethoxy) ethoxy]ethoxy]ethoxy]ethylsulfamoyl]anilino]pyrimidin-4-yl]amino]-6-fluoro-benzamide (2 g, 2.85 mmol, 65.67% yield) was obtained as a yellow solid.

Step 4. To a solution of 2-[[5-bromo-2-[4-[2-[2-[2-[2-(2-hydroxyethoxy)ethoxy]ethoxy]ethoxy]ethylsulfamoyl]anilino]pyrimidin-4-yl]amino]-6-fluoro-benzamide (1 g, 1.43 mmol, 1 equiv), TEA (552 mg, 4.28 mmol, 750 μL, 3 equiv) in THF (20 mL) was added TosCl (326 mg, 1.71 mmol, 1.2 equiv) at 20° C. The mixture was stirred at 20° C. for 12 hours. LCMS showed that there was 48% of the desired MS detected. The mixture was diluted with EtOAc (60 mL) and washed by water (40 mL), brine (40 mL), the organic layer was dried over Na₂SO₄, concentrated by vacuo. The residue was purified by prep-HPLC (FA,column: Phenomenex luna C18 150*40 mm*15 m; mobile phase: [water(FA)-ACN]; B%: 44%-74%, 10 min). The product 2-[2-[2-[2-[2-[[4-[[5-bromo-4-(2-carbamoyl-3-fluoro-anilino)pyrimidin-2-yl]amino]phenyl]sulfonylamino]ethoxy]ethoxy]ethoxy]ethoxy]ethyl 4-methylbenzenesulfonate (570 mg, 666.09 μmol, 46.73% yield) was obtained as a yellow solid.

Step 5. To a solution of 2-[2-[2-[2-[2-[[4-[[5-bromo-4-(2-carbamoyl-3-fluoro-anilino)pyrimidin-2-yl]amino]phenyl]sulfonylamino]ethoxy]ethoxy]ethoxy]ethoxy]ethyl-4-methyl benzenesulfonate (520 mg, 607.66 μmol, 1 equiv), 3-methoxy-4-nitro-1H-pyrazole (94 mg, 656.88 μmol, 1.08 equiv) in DMF (5 mL) was added Cs₂CO₃ (257 mg, 789.95 μmol, 1.3 equiv) at 20° C. The mixture was stirred at 100° C. for 1 h. LCMS showed that there was one main peak with the desired MS detected. The mixture was diluted with EtOAc (50 mL) and washed by water (50 mL), brine (50 mL), the organic layer was dried over Na₂SO₄, concentrated by vacuo. The residue was purified by prep-HPLC (FA, column: Phenomenex luna C18 150*40 mm*15 m; mobile phase: [water(FA)-ACN]; B%: 37%-67%, 10 min). The product 2-[[5-bromo-2-[4-1[2-[2-1[2-[2-[2-(3-methoxy-4-nitro-pyrazol-1-yl)ethoxy]ethoxy]ethoxy]ethoxy]ethylsulfamoyl]anilino]pyrimidin-4-yl]amino]-6-fluoro-benzamide (400 mg, 483.88 μmol, 79.63% yield) was obtained as a yellow solid.

Step 6. 2-[[5-bromo-2-[4-[1[2[2-[12-[2-(3-methoxy-4-nitro-pyrazol-1-yl)ethoxy]ethoxy]ethoxy]ethoxy]ethylsulfamoyl]anilino]pyrimidin-4-yl]amino]-6-fluoro-benzamide (350 mg, 423.40 μmol, 1 equiv), Fe (47 mg, 846.80 μmol, 2 eq), NH₄Cl (113 mg, 2.12 mmol, 5 equiv) in EtOH (10 mL), H₂O (2 mL) was stirred at 80° C. for 12 h under N2. LCMS showed that there was one main peak with the desired MS detected. The mixture was filtered, and the filtrate was poured into NaHCO₃ (aq.) (50 mL), EtOAc (50 mL) were added and layers were separated. The aqueous phase was extracted with EtOAc (30 mL). Combined extracts were washed with brine (50 mL), dried over Na₂SO₄, filtered, and concentrated under vacuum to give a residue. The residue was purified by prep-HPLC (FA, column: Phenomenex Synergi C18 150*25 mm*10% m; mobile phase: [water(FA)-ACN]; B%: 15%-45%, 10 min). The product 2-[[2-[4-[2-1[2-[2-1[2-[2-(4-amino-3-methoxy-pyrazol-1-yl)ethoxy]ethoxy]ethoxy]ethoxy]ethylsulfamoyl]anilino]-5-bromo-pyrimidin-4-yl]amino]-6-fluoro-benzamide (130 mg, 163.18 μmol, 38.54% yield) was obtained as a yellow solid.

Step 7. To a solution of 2-[[2-[4-[2-[2-[2-[2-[2-(4-amino-3-methoxy-pyrazol-1-yl)ethoxy]ethoxy]ethoxy]ethoxy]ethylsulfamoyl]anilino]-5-bromo-pyrimidin-4-yl]amino]-6-fluoro-benzamide (120 mg, 150.63 μmol, 1 equiv), DIEA (58 mg, 451.89 μmol, 78.71 μL, 3 equiv) in DMA (1 mL) was added 2,6-dichloro-9-methyl-purine (36 mg, 180.75 μmol, 1.2 equiv) at 20° C. The mixture was stirred at 120° C. for 2 h. LCMS showed that there was one main new peak with the desired MS detected. The mixture was filtered, and the filtrate was concentrated to give a residue. The residue was purified by prep-HPLC (FA, column: Phenomenex Synergi C18 150*25 mm*10 m; mobile phase: [water(FA)-ACN]; B%: 40%-58%, 9 min). The product 2-[[5-bromo-2-[4-[2-[2-[2-[2-[2-[4-[(2-chloro-9-methyl-purin-6-yl)amino]-3-methoxy-pyrazol-1 yl]ethoxy]ethoxy]ethoxy]ethoxy]ethylsulfamoyl]anilino]pyrimidin-4-yl]amino]-6-fluoro-benzamide (100 mg, 103.82 μmol, 68.92% yield) was obtained as a yellow oil.

Step 8. To a solution of 2-[[5-bromo-2-[4-[2-[2-[2-[2-[2-[4-[(2-chloro-9-methyl-purin-6-yl)amino]-3-methoxy-pyrazol-1-yl]ethoxy]ethoxy]ethoxy]ethoxy]ethylsulfamoyl]anilino]pyrimidin-4-yl]amino]-6-fluoro-benzamide (90 mg, 93.44 μmol, 1 equiv) tert-butyl N-[(3R,4R)-4-fluoropyrrolidin-3-yl]carbamate (38 mg, 186.87 μmol, 2 equiv) in n-BuOH (2 mL) was added DIEA (27.00 mg, 208.91 μmol, 36.39 μL, 2.24 equiv) at 20° C. The mixture was stirred at 120° C. for 12 h. LCMS showed that there was 38% of the desired MS detected. The mixture was concentrated by vacuo to give a residue. The residue was purified by prep-HPLC (neutral, column: Waters Xbridge 150*25 mm*5 m; mobile phase: [water (NH₄HCO₃)-ACN]; B%: 38%-68%, 9 min). The product tert-butyl N-[(3R,4R)-1-[6-[[1-[2-[2-[2-[2-[2-[[4-[[5-bromo-4-(2-carbamoyl-3-fluoro-anilino)pyrimidin-2-yl]amino]phenyl]sulfonylamino]ethoxy]ethoxy]ethoxy]ethoxy]ethyl]-3-methoxy-pyrazol-4-yl]amino]-9-methyl-purin-2-yl]-4-fluoro-pyrrolidin-3-yl]carbamate (30 mg, 26.52 μmol, 28.39% yield) was obtained as a yellow solid.

Step 9. To a solution of tert-butyl N-[(3R,4R)-1-[6-[[1-[2-[2-[2-[2-[2-[[4-[[5-bromo-4-(2-carbamoyl-3-fluoro-anilino)pyrimidin-2-yl]amino]phenyl]sulfonylamino]ethoxy]ethoxy]ethoxy]ethoxy]ethyl]-3-methoxy-pyrazol-4-yl]amino]-9-methyl-purin-2-yl]-4-fluoro-pyrrolidin-3-yl]carbamate (20 mg, 17.68 μmol, 1 equiv) in DCM (2 mL) was added TFA (513 mg, 4.50 mmol, 330 μL, 254.59 equiv) at 20° C. The mixture was stirred at 20° C. for 1 h. LCMS showed that there was one main peak with the desired MS detected. The reaction mixture was concentrated by vacuo to give a residue. The residue was used for the next step without the further purification. The product 2-[[2-[4-[2[2[2-[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy]ethoxy]ethoxy]ethylsulfamoyl]anilino]-5-bromo-pyrimidin-4-yl]amino]-6-fluoro-benzamide (20 mg, crude, TFA) was obtained as a yellow oil. To a solution of 2-[[2-[4-[2-[2-[2-[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy]ethoxy]ethoxy]ethylsulfamoyl]anilino]-5-bromo-pyrimidin-4-yl]amino]-6-fluoro-benzamide (20 mg, 17.47 μmol, 1 eq, TFA), DIEA (7 mg, 52.41 μmol, 9.13 μL, 3 eq) in THF (1 mL) was added prop-2-enoyl prop-2-enoate (3 mg, 20.96 μmol, 1.2 eq) at 20° C. The mixture was stirred at 20° C. for 1 hour. LCMS showed that the reaction was too messy and no the desired Ms detected. The reaction mixture was concentrated by vacuo to give a residue. The residue was purified by prep-HPLC (FA, column: Unisil 3-100 C18 Ultra 150*50 mm*3 m; mobile phase: [water(FA)-ACN]; B%: 28%-58%, 10 min). The product 2-[[5-bromo-2-[4-[2-[2-[2-[2-[2-[4-[[2-[(3R,4R)-3-fluoro-4-(prop-2-enoylamino)pyrrolidin-1-yl]-9-methyl-purin-6-yl]amino]-3-methoxy-pyrazol-1-yl]ethoxy]ethoxy]ethoxy]ethoxy]ethylsulfamoyl]anilino]pyrimidin-4-yl]amino]-6-fluoro-benzamide (6.59 mg, 5.95 μmol, 34.04% yield, 97.9% purity) was obtained as a white solid, and its structure was confirmed by HNMR, Q-TOF, QC LCMS. ¹H NMR (400 MHz, CDCl₃) δ11.43 (s, 1H), 8.40-8.34 (m, 1H), 8.23 (s, 1H), 7.99 (s, 1H), 7.71-7.62 (m, 3H), 7.61-7.54 (m, 2H), 7.46-7.42 (m, 1H), 7.42-7.34 (m, 1H), 7.15 (s, 1H), 7.11-7.01 (m, 1H), 6.98-6.88 (m, 1H), 6.88-6.78 (m, 1H), 6.45-6.30 (m, 2H), 6.28-6.18 (m, 1H), 6.14-6.06 (m, 1H), 5.67-5.61 (m, 1H), 5.34-5.08 (m, 1H), 4.78-4.58 (m, 1H), 4.17-4.09 (m, 2H), 4.00-3.69 (m, 1OH), 3.64-3.48 (m, 1OH) 3.47-3.40 (m, 8H), 3.13-3.06 (m, 2H). LC-MS: MS (ES′): RT=2.338 min, m/z=1088.1 [M+H⁺].

### Example 123—Synthesis of Compound I-139

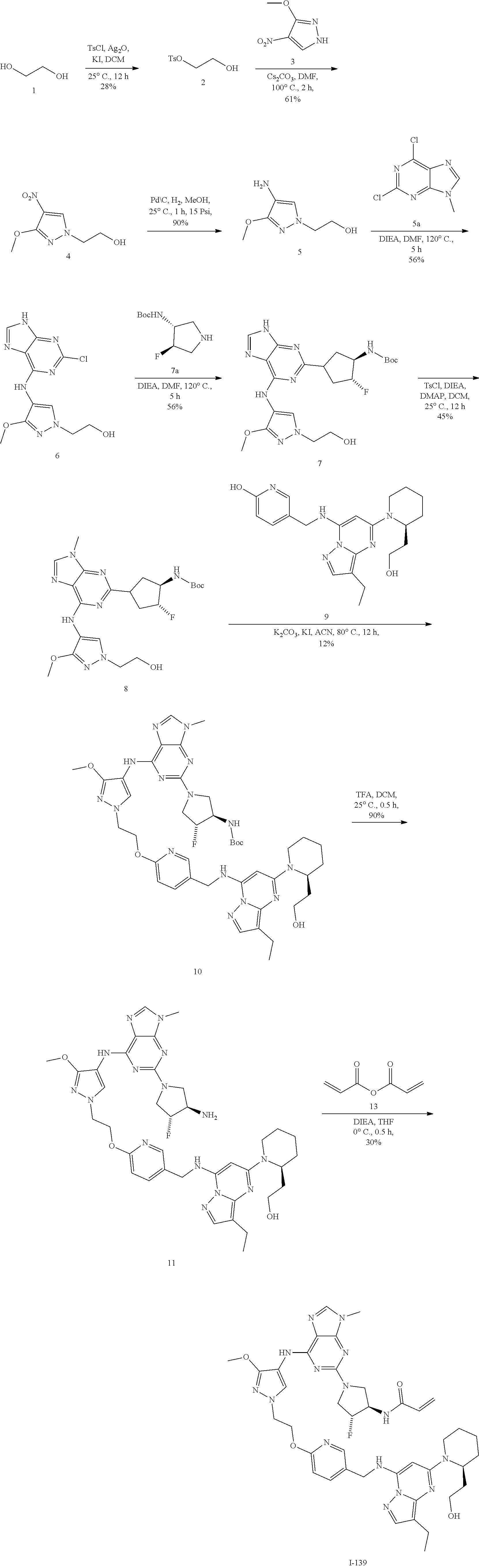

Step 1. To a solution of ethylene glycol (5 g, 80.56 mmol, 4.50 mL, 1.0 equiv) in DCM (100 mL) were added Ag₂0 (28 g, 120.84 mmol, 1.5 equiv), KI (2.7 g, 16.26 mmol, 2.02e⁻¹ equiv) and TosCl (17 g, 89.17 mmol, 1.1 equiv). The mixture was stirred at 25° C. for 12 hours. The reaction mixture was filtered and concentrated under reduced pressure to give a residue. The residue was purified by column chromatography (SiO₂, PE/EtOAc=I/O to 1/1). Compound 2-hydroxyethyl 4-methylbenzenesulfonate (5 g, 23.12 mmol, 28.70% yield) (Rf=0.25) was obtained as a colorless oil. ¹H-NMR confirmed the obtained product. ¹H NMR: (400 MHz, DMSO-d₆) δ7.80 (d, J=8.4 Hz, 2H), 7.49 (d, J=8.0 Hz, 2H), 4.98 (br s, 1H), 4.02-3.97 (m, 2H), 3.55 (t, J=4.8 Hz, 2H), 2.43 (s, 3H).

Step 2. To a solution of 2-hydroxyethyl 4-methylbenzenesulfonate (1.5 g, 6.94 mmol, 1 equiv) in DMF (10 mL) were added Cs₂CO₃ (2.7 g, 8.29 mmol, 1.2 equiv) and 3-methoxy-4-nitro-1H-pyrazole (825 mg, 5.77 mmol, 8.31e⁻¹ equiv). The mixture was stirred at 100° C. for 2 hours. The reaction mixture was quenched by addition H₂O (20.0 mL), and then extracted with EtOAc 40.0 mL (20.0 mL×2). The combined organic layers were washed with brine 60.0 mL (20.0 mL*3), dried over anhydrous Na₂SO₄, filtered and concentrated under reduced pressure to give a residue. The residue was purified by column chromatography (SiO₂, Petroleum ether/Ethyl acetate=I/O to 1/1). Compound 2-(3-methoxy-4-nitro-pyrazol-1-yl)ethanol (0.8 g, 4.27 mmol, 61.63% yield) (Rf=0.2) was obtained as a white solid. ¹H-NMR confirmed the obtained product. ¹H NMR: (400 MHz, DMSO-d₆): δ (ppm) 8.65 (s, 1H), 4.98 (t, J=5.4 Hz, 1H), 4.08-4.04 (m, 2H), 3.95 (s, 3H), 3.76-3.71 (m, 2H).

Step 3. To a solution of Pd/C (0.075 g, 10% purity) in MeOH (10 mL) was added 2-(3-methoxy-4-nitro-pyrazol-1-yl)ethanol (0.75 g, 4.01 mmol, 1 equiv) under N₂. The suspension was degassed under vacuum and purged with H₂ several times. The mixture was stirred under H₂ (15 psi) at 25° C. for 1 hour. The reaction mixture filtered and concentrated under reduced pressure to give a residue. Compound 2-(4-amino-3-methoxy-pyrazol-1-yl) ethanol (0.65 g, crude) (Rf=0.1) was obtained as a yellow solid. ¹H-NMR confirmed the obtained product. ¹H NMR: (400 MHz, DMSO-d₆): δ (ppm) 6.94 (s, 1H), 4.76 (t, J=5.2 Hz, 1H), 3.81-3.78 (m, 2H), 3.74 (s, 3H), 3.60 (q, J=5.6 Hz, 2H).

Step 4. To a solution of 2-(4-amino-3-methoxy-pyrazol-1-yl) ethanol (0.65 g, 4.14 mmol, 1 equiv) in DMF (3 mL) were added DIEA (1.5 g, 12.06 mmol, 2.1 mL, 2.9 equiv) and 2,6-dichloro-9-methyl-purine (0.78 g, 3.84 mmol, 9.29e⁻¹ equiv). The mixture was stirred at 120° C. for 5 hours. The reaction mixture was filtered and concentrated under reduced pressure to give a residue. The residue was purified by prep-HPLC (FA condition). (column: Phenomenex C18 75*30 mm*3 m; mobile phase: [water(FA)-ACN]; B%: 12%-42%, 7 min). Compound 2-[4-[(2-chloro-9-methyl-purin-6-yl) amino]-3-methoxy-pyrazol-1-yl]ethanol (0.75 g, 2.32 mmol, 56.02% yield) was obtained as a brown solid. ¹H-NMR confirmed the obtained product. ¹H NMR: (400 MHz, DMSO-d₆): δ (ppm) 9.24 (br s, 1H), 8.18 (br s, 1H), 7.71 (br s, 1H), 4.91 (br s, 1H), 3.99 (t, J=5.6 Hz, 2H), 3.79 (br s, 3H), 3.71 (br s, 5H).

Step 5. To a solution of 2-[4-[(2-chloro-9-methyl-purin-6-yl)amino]-3-methoxy-pyrazol-1-yl]ethanol (220 mg, 679.56 μmol, 1.0 equiv) in n-BuOH (2 mL) were added tert-butyl N-[(3R,4R)-4-fluoropyrrolidin-3-yl]carbamate (0.3 g, 1.47 mmol, 2.1 equiv) and DIEA (185 mg, 1.44 mmol, 0.25 mL, 2.1 equiv). The mixture was stirred at 120° C. for 12 h. The reaction mixture was concentrated under reduced pressure to give a residue. The residue was purified by prep-HPLC (FA condition). (column: Phenomenex luna C18 150*40 mm*15 m; mobile phase: [water(FA)-ACN]; B%: 20%-50%, 10 min). Compound tert-butyl N-[(3R,4R)-4-fluoro-1-[6-[[1-(2-hydroxyethyl)-3-methoxy-pyrazol-4-yl]amino]-9-methyl-purin-2-yl]pyrrolidin-3-yl]carbamate (0.19 g, 386.56 μmol, 56.88% yield) was obtained as a brown solid. ¹H-NMR confirmed the obtained product. ¹H NMR: (400 MHz, DMSO-d₆): δ (ppm) 7.98 (s, 1H), 7.92 (s, 1H), 7.79 (s, 1H), 7.37 (br d, J=5.6 Hz, 1H), 5.18-4.97 (m, 1H), 4.86 (br d, J=2.8 Hz, 1H), 4.20-4.08 (m, 1H), 4.00 (t, J=5.6 Hz, 2H), 3.91-3.65 (m, 8H), 3.60-3.52 (m, 1H), 3.61 (s, 4H), 1.40 (s, 9H).

Step 6. To a solution of tert-butyl N-[(3R,4R)-4-fluoro-1-[6-[[1-(2-hydroxyethyl)-3-methoxy-pyrazol-4-yl]amino]-9-methyl-purin-2-yl]pyrrolidin-3-yl]carbamate (170 mg, 345.87 μmol, 1 equiv) in DCM (3 mL) were added TosCl (140 mg, 734.34 μmol, 2.2 equiv) DMAP (10 mg, 81.85 μmol, 2.37e⁻¹ equiv) and DIEA (148 mg, 1.15 mmol, 0.2 mL, 3.3 equiv). The mixture was stirred at 25° C. for 12 h. The reaction mixture was concentrated under reduced pressure to give a residue. The residue was purified by prep-HPLC (FA condition). (column: Phenomenex C18 75*30 mm*3 m; mobile phase: [water(FA)-ACN]; B%: 40%-70%, 7 min) Compound 2-[4-[[2-[(3R,4R)-3-(tert-butoxycarbonylamino)-4-fluoro-pyrrolidin-1-yl]-9-methyl-purin-6-yl]amino]-3-methoxy-pyrazol-1-yl]ethyl 4-methylbenzenesulfonate (100 mg, 154.87 μmol, 44.78% yield) was obtained as a white solid. ¹H-NMR confirmed the obtained product. ¹H NMR: (400 MHz, DMSO-d₆): δ (ppm) 7.99 (s, 1H), 7.83 (d, J=14.4 Hz, 2H), 7.61 (d, J=8.4 Hz, 2H), 7.40-7.37 (m, 1H), 7.35 (d, J=8.0 Hz, 2H), 5.17-5.00 (m, 1H), 4.30-4.25 (m, 2H), 4.22 (br d, J=4.0 Hz, 2H), 4.18-4.11 (m, 1H), 3.83 (br s, 1H), 3.91-3.72 (m, 3H), 3.71 (s, 3H), 3.63 (s, 3H), 3.59 (br d, J=12.0 Hz, 1H), 2.34 (s, 3H), 1.39 (s, 9H).

Step 7. To a solution of 2-[4-[[2-[(3R,4R)-3-(tert-butoxycarbonylamino)-4-fluoro-pyrrolidin-1-yl]-9-methyl-purin-6-yl]amino]-3-methoxy-pyrazol-1-yl]ethyl4-methyl benzenesulfonate (90 mg, 139.38 μmol, 1.0 equiv) in CH₃CN (2 mL) were added 5-[[[3-ethyl-5-[(2S)-2-(2-hydroxyethyl)-1-piperidyl]pyrazolo[1,5-a]pyrimidin-7-yl]amino]methyl]pyridin-2-ol (63 mg, 158.90 μmol, 1.1 equiv), KI (27 mg, 162.65 μmol, 1.2 equiv) and K₂CO₃ (45 mg, 325.60 μmol, 2.3 equiv). The mixture was stirred at 80° C. for 12 h. The reaction mixture was filtered and concentrated under reduced pressure to give a residue. The residue was purified by prep-HPLC (FA condition). (column: Unisil 3-100 C18 Ultra 150*50 mm*3 m; mobile phase: [water(FA)-ACN]; B%: 22%-52%, 10 min). Compound tert-butyl N-[(3R,4R)-1-[6-[[1-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethyl]-3-methoxy-pyrazol-4-yl]amino]-9-methyl-purin-2-yl]-4-fluoro-pyrrolidin-3-yl]carbamate (15 mg, 17.24 μmol, 12.37% yield) was obtained as a white solid. ¹H NMR: (400 MHz, DMSO-d₆): δ (ppm) 8.25 (d, J=2.0 Hz, 1H), 8.00 (s, 1H), 7.93 (s, 1H), 7.84 (t, J=6.4 Hz, 1H), 7.80-7.72 (m, 2H), 7.65 (s, 1H), 7.36 (br d, J=5.2 Hz, 1H), 6.76 (d, J=8.4 Hz, 1H), 5.59 (s, 1H), 5.16-4.95 (m, 1H), 4.72 (br s, 1H), 4.61-4.52 (m, 3H), 4.47 (br d, J=6.4 Hz, 2H), 4.33 (br t, J=5.2 Hz, 2H), 4.30-4.21 (m, 1H), 4.16-4.08 (m, 1H), 3.84-3.71 (m, 6H), 3.61 (s, 3H), 3.55 (br d, J=11.2 Hz, 1H), 2.86-2.77 (m, 1H), 2.49-2.44 (m, 2H), 1.86-1.76 (m, 1H), 1.69-1.54 (m, 6H), 1.36 (s, 9H), 1.17 (t, J=7.6 Hz, 3H).

Step 8. To a solution of tert-butyl N-[(3R,4R)-1-[6-[[1-[2-[[5-[[[3-ethyl-5-[(2S)-2-(2-hydroxyethyl)-1-piperidyl]pyrazolo[ 1,5-a]pyrimidin-7-yl]amino]methyl]-2-pyridyl]oxy]ethyl]-3-methoxy-pyrazol-4-yl]amino]-9-methyl-purin-2-yl]-4-fluoro-pyrrolidin-3-yl]carbamate (10 mg, 11.49 μmol, 1.0 equiv) in DCM (2 mL) was added TFA (3 mg, 22.99 μmol, 1.70 μL, 2.0 equiv). The mixture was stirred at 25° C. for 0.5 h. The reaction mixture was concentrated under reduced pressure to give a residue. Compound 2-[(2S)-1-[7-[[6-[2-[4-[[2-[(3R,4R)-3-amino-4-fluoro-pyrrolidin-1-yl]-9-methyl-purin-6-yl]amino]-3-methoxy-pyrazol-1-yl]ethoxy]-3-pyridyl]methylamino]-3-ethyl pyrazolo [1, 5-a]pyrimidin-5-yl]-2-piperidyl]ethanol (10 mg, crude) was obtained as a colorless oil.

Step 9. To a solution of 2-[(2S)-1-[7-[[6-[2-[4-[[2-[(3R,4R)-3-amino-4-fluoro-pyrrolidin-1-yl]-9-methyl-purin-6-yl]amino]-3-methoxy-pyrazol-1-yl]ethoxy]-3-pyridyl]methylamino]-3-ethyl-pyrazolo[1,5-a]pyrimidin-5-yl]-2-piperidyl]ethanol (10 mg, 12.99 μmol, 1 equiv) and DIEA (5 mg, 38.69 μmol, 6.74 μL, 3.0 equiv) in THF (3 mL) was added prop-2-enoyl prop-2-enoate (3 mg, 19.82 μmol, 1.53 equiv). The mixture was stirred at 0° C. for 0.5 h. The reaction mixture was concentrated under reduced pressure to give a residue. The residue was purified by prep-HPLC (neutral condition). (column: Waters Xbridge 150*25 mm*5 m; mobile phase: [water(NH₄HCO₃)-ACN]; B%: 38%-68%, 8 min). Compound N-[(3R,4R)-1-[6-[[1-[2-[[5-1[[[3-ethyl-5-[(2S)-2-(2-hydroxyethyl)-1-piperidyl]pyrazolo[1,5-a]pyrimidin-7-yl]amino]methyl]-2-pyridyl]oxy]ethyl]-3-methoxy-pyrazol-4-yl]amino]-9-methyl-purin-2-yl]-4-fluoro-pyrrolidin-3-yl]prop-2-enamide (3.25 mg, 3.94 μmol, 30.37% yield, 100% purity) was obtained as a white solid. ¹H-NMR, QTOF, and QC LCMS data confirmed the obtained product. ¹H NMR: (400 MHz, CDCl₃):6(ppm) 8.06 (d, J=2.0 Hz, 1H), 7.87 (s, 1H), 7.55 (s, 1H), 7.51 (dd, J=8.8, 2.4 Hz, 1H), 7.37 (s, 1H), 7.07 (s, 1H), 6.65 (d, J=8.4 Hz, 1H), 6.46-6.36 (m, 1H), 6.33-6.25 (m, 2H), 6.09-6.00 (m, 1H), 5.73-5.61 (m, 1H), 5.59 (d, J=10.4 Hz, 1H), 5.27 (s, 1H), 5.14-4.98 (m, 2H), 4.57 (t, J=4.8 Hz, 2H), 4.53-4.45 (m, 1H), 4.38 (d, J=5.6 Hz, 2H), 4.29 (t, J=5.2 Hz, 2H), 3.90 (s, 3H), 3.81-3.75 (m, 2H), 3.73-3.64 (m, 3H), 3.56 (s, 3H), 3.55-3.50 (m, 1H), 3.27 (br t, J=11.2 Hz, 1H), 3.00-2.91 (m, 1H), 2.63-2.51 (m, 2H), 2.06-1.94 (m, 1H), 1.77-1.59 (m, 5H), 1.20-1.15 (m, 4H). LC-MS: MS (ES⁺): RT=2.298 min, m/z=824.3 [M+H⁺]; LCMS Method: 10.

### Example 124—Synthesis of Compound I-140

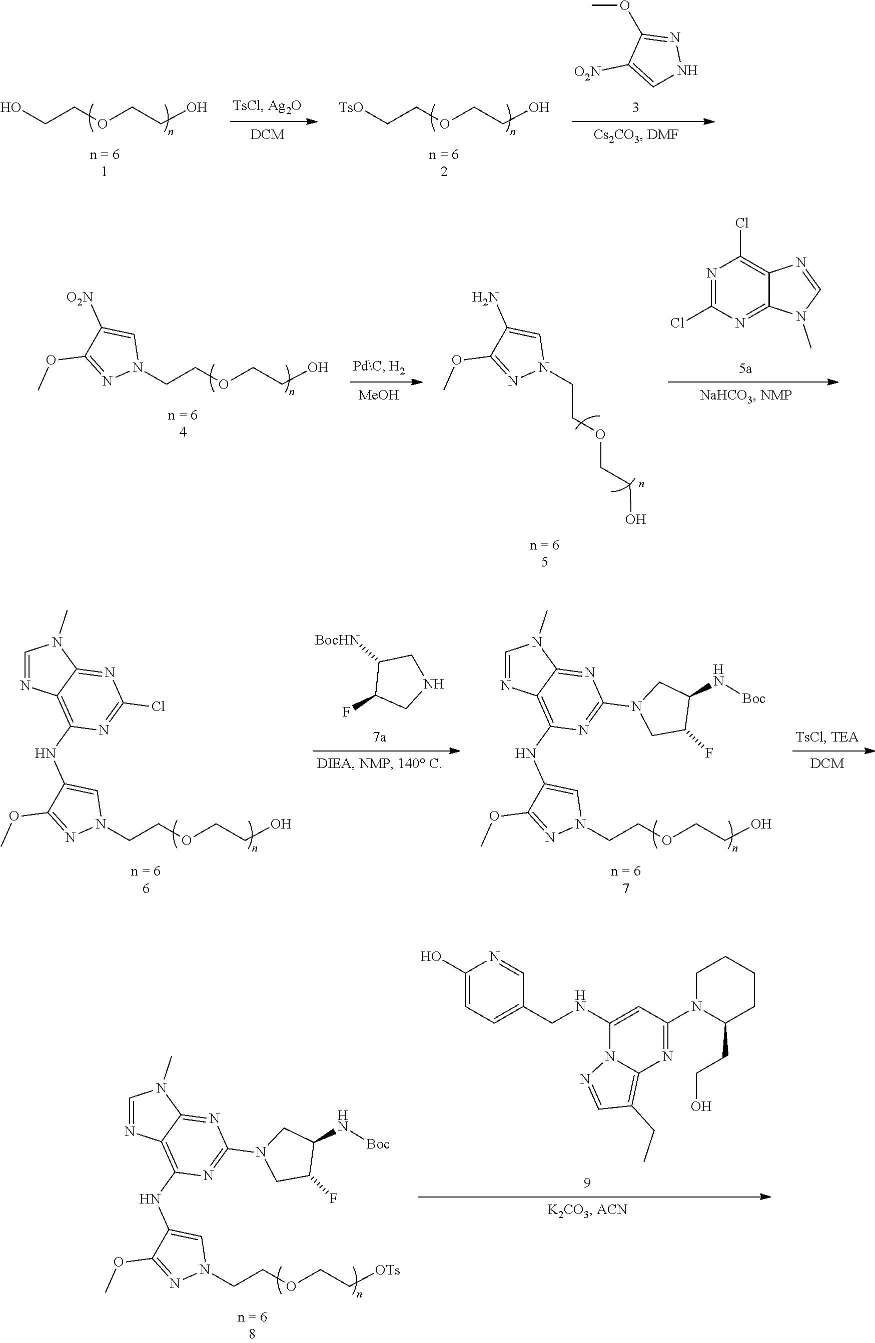

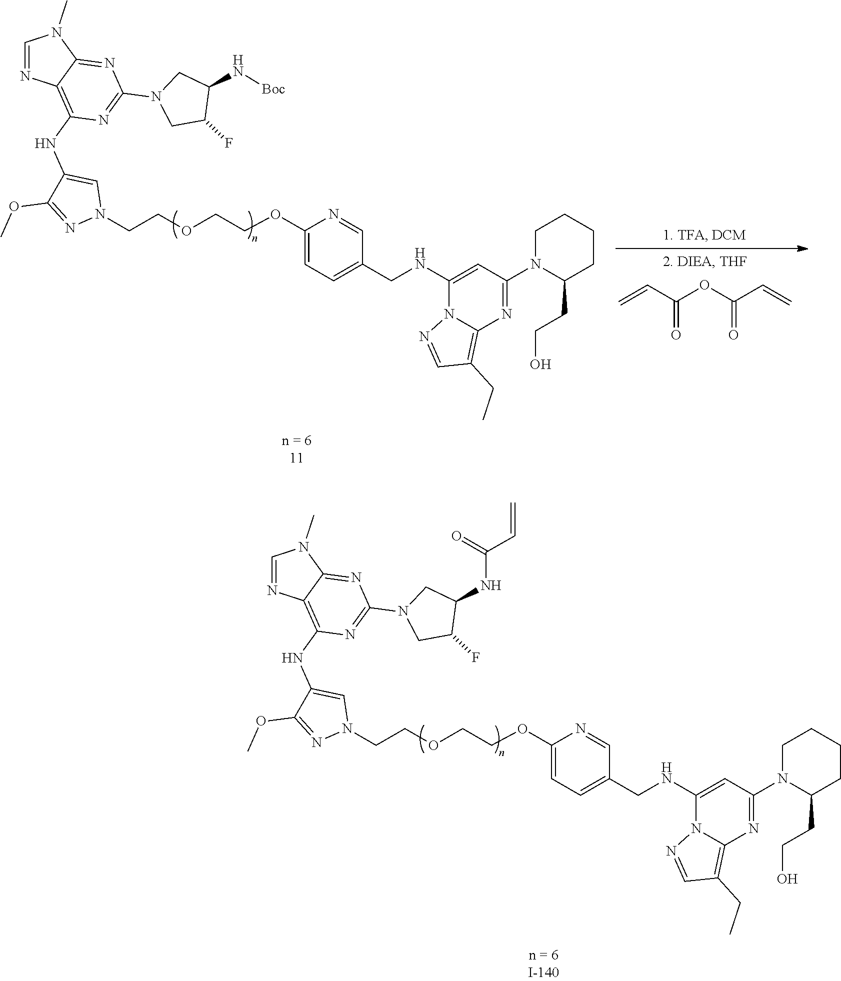

Step 1. To a solution of 2-[2-[2-[2-[2-[2-(2-hydroxyethoxy)ethoxy]ethoxy]ethoxy]ethoxy]ethoxy]ethanol (5 g, 15.32 mmol, 1 equiv) in DCM (80 mL) was added KI (3 g, 18.07 mmol, 1.18 equiv), Ag₂O (5.3 g, 22.87 mmol, 1.49 equiv) and 4-methylbenzenesulfonyl chloride (3 g, 15.74 mmol, 1.03 equiv). The mixture was stirred at 0-25° C. for 10 h. TLC (DCM /MeOH=10/1) showed the starting material was consumed completely and a main new spot with lower polarity was observed. The mixture was filtered and the filtrate was concentrated to give a residue. It was purified by silica gel chromatography column (Petroleum ether/EtOAc=1/1− DCM/MeOH=10/1). Compound 2-[2-[2-[2-[2-[2-(2-hydroxyethoxy)ethoxy]ethoxy]ethoxy]ethoxy]ethoxy]ethyl 4-methylbenzenesulfonate (6.2 g, 12.90 mmol, 84.22% yield) was obtained as a colorless oil. ¹H NMR (400 MHz, CDCl₃) δ6.93-6.81 (m, 1H), 6.73-6.58 (m, 2H), 1.36-1.27 (m, 3H), 1.13-1.12 (m, 6H), 1.11-1.10 (m, 6H), 1.06 (s, 6H).

Step 2. To a solution of 2-[2-[2-[2-[2-[2-(2-hydroxyethoxy)ethoxy]ethoxy]ethoxy]ethoxy]ethoxy]ethyl 4-methylbenzenesulfonate (200 mg, 416.17 μmol, 1 equiv) in DMF (5 mL) was added Cs₂CO₃ (280.00 mg, 859.37 μmol, 2.06 equiv) and 3-methoxy-4-nitro-1Hpyrazole (60.00 mg, 419.29 μmol, 1.01 equiv). The mixture was stirred at 100° C. for 2 h. LCMS showed the starting material was consumed completely and the desired MS was detected. The mixture was filtered and the filtrate was collected. It was purified with the batch of by prep-HPLC (column: Phenomenex luna C18 150*40 mm*15 m; mobile phase: [water(FA)-ACN]; B%: 12%-42%, 10 min). Compound 2-[2-[2-[2-[2-[2-[2-(3-methoxy-4-nitro-pyrazol-1-yl)ethoxy]ethoxy]ethoxy]ethoxy]ethoxy]ethoxy]ethanol (850 mg) as ayellow oil. ¹H NMR (400 MHz, CHLOROFORM-d) 6 ppm 3.59-3.53 (m, 23H), 3.66-3.64 (m, 2H), 3.78-3.76 (m, 2H), 3.96 (s, 3H), 4.10-4.08 (m, 2H), 8.17 (s, 1H).

Step 3. To a solution of Pd/C (0.05 g, 10% purity) in THF (10 mL) was added 2-[2-[2-[2-[2-[2-[2-(3-methoxy-4-nitro-pyrazol-1-yl)ethoxy]ethoxy]ethoxy]ethoxy]ethoxy]ethoxy]ethanol (750 mg, 1.66 mmol, 1 equiv) under N₂ atmosphere. The suspension was degassed and purged with H₂ for 3 times. The mixture was stirred under H₂ (15 Psi.) at 25° C. for 2 hr. LCMS showed the starting material was consumed completely and the desired MS was detected. The reaction mixture was filtered and the filtrate was concentrated to give the desired product. N/A. The crude product 2-[2-[2-[2-[2-[2-[2-(4-amino-3-methoxy-pyrazol-1-yl)ethoxy]ethoxy]ethoxy]ethoxy]ethoxy]ethoxy]ethanol (650 mg, 1.54 mmol, 92.83% yield) as a light yellow oil was used into the next step without further purification.

Step 4. To a solution of 2-[[2[2-[2-[2-[2-(4-amino-3-methoxy-pyrazol-1-yl)ethoxy]ethoxy]ethoxy]ethoxy]ethoxy]ethoxy]ethanol (650 mg, 1.54 mmol, 1 equiv) in DMF (5 mL) was added DIPEA (482.30 mg, 3.73 mmol, 650.00 μL, 2.42 equiv) and 2,6-dichloro-9-methyl-purine (500 mg, 2.46 mmol, 1.60 equiv). The mixture was stirred at 120° C. for 3 h. LCMS showed the starting material was consumed completely and the desired MS was detected. Solvent was removed to give a residue. It was purified by prep-HPLC (column: Phenomenex luna C18 150*40 mm*15 m; mobile phase: [water (FA)-ACN]; B%: 18%-48%, 10 min). Compound 2-[21[2-[2-[2-[2-[4-[(2-chloro-9-methyl-purin-6-yl)amino]-3-methoxy-pyrazol-1-yl]ethoxy]ethoxy]ethoxy]ethoxy]ethoxy]ethoxy]ethanol (520 mg, 884.28 μmol, 57.34% yield) was obtained as a yellow oil.

Step 5. To a solution of 2-[2-[2-[2-[2-[2-[2-[4-[(2-chloro-9-methyl-purin-6-yl)amino]-3-methoxy-pyrazol-1-yl]ethoxy]ethoxy]ethoxy]ethoxy]ethoxy]ethoxy]ethanol (520 mg, 884.28 μmol, 1 equiv) in n-BuOH (10 mL) was added DIPEA (371.00 mg, 2.87 mmol, 0.5 mL, 3.25 equiv) and tert-butyl N-[(3R,4R)-4-fluoropyrrolidin-3-yl]carbamate (304 mg, 1.49 mmol, 1.68 equiv). The mixture was stirred at 120° C. for 10 h. LCMS showed 14% starting material remained and about 82% desired MS was detected. Solvent was removed to give a residue. It was purified by prep-HPLC (column: Phenomenex luna C18 150*40 mm*15 m; mobile phase: [water (FA)-ACN]; B%: 23%-53%, 10 min). Compound tert-butyl N-[(3R, 4R)-4-fluoro-1-[6-[[1-1[2-1[[2-[2-1[2-1[2(2hydroxyethoxy)ethoxy]ethoxy]ethoxy]ethoxy]ethoxy]ethyl]-3-methoxy-pyrazol-4-yl]amino]-9-methyl-purin-2-yl]pyrrolidin-3-yl]carbamate (360 mg, 476.30 μmol, 53.86% yield) was obtained as a brown oil.

Step 6. To a solution of tert-butyl N-[(3R,4R)-4-fluoro-1-[6-[[1-[2-[2-[2-[2-[2-[2-(2-Hydroxyethoxy)ethoxy]ethoxy]ethoxy]ethoxy]ethoxy]ethyl]-3-methoxy-pyrazol-4-yl]amino]-9-methyl-purin-2-yl]pyrrolidin-3-yl]carbamate (340 mg, 449.84 μmol, 1 equiv) in DCM (8 mL) was added DIPEA (126.14 mg, 975.99 μmol, 170.00 μL, 2.17 equiv), DMAP (20 mg, 163.71 μmol, 3.64e⁻¹ eq) and 4-methylbenzenesulfonyl chloride (150 mg, 786.79 μmol, 1.75 equiv) at 0° C. The mixture was stirred at 0-25° C. for 10 h. LCMS showed the starting material was consumed and the desired MS was detected. Solvent was removed to give a residue. It was purified by prep-HPLC (column: Phenomenex luna C18 150*40 mm*15 m; mobile phase: [water (FA)-ACN]; B%: 44%-74%, 10 min). Compound 2-[2-[2-[2-[2-[2-[2-[4-[[2-[(3R,4R)-3-(tert-butoxycarbonylamino) -4-fluoro-pyrrolidin-1-yl]-9-methyl-purin-6-yl]amino]-3-methoxy-pyrazol-1-yl]ethoxy]ethoxy]ethoxy]ethoxy]ethoxy]ethoxy]ethyl 4-methylbenzenesulfonate (240 mg, 263.73 μmol, 58.63% yield) was obtained as a yellow oil. ¹H NMR (400 MHz, CHLOROFORM-d) 6 ppm 7.92 (s, 1H), 7.77-7.65 (m, 3H), 7.26 (d, 2H, J=8.13 Hz), 5.19-5.04 (m, 1H), 4.30 (s, 1H), 4.07 (t, 4H, J=4.82 Hz), 3.89 (s, 3H), 3.84-3.73 (m, 5H), 3.64 (s, 3H), 3.60 (d, 2H, J=4.88 Hz), 3.56-3.41 (m, 23H), 2.37 (s, 3H), 1.38 (s, 9H).

Step 7. To a solution of 2-[2-[2-[2-[2-[2-[2-[4-[[2-[(3R,4R)-3-(tert-butoxycarbonyl amino)-4-fluoro-pyrrolidin-1-yl]-9-methyl-purin-6-yl]amino]-3-methoxy-pyrazol-1-yl]ethoxy]ethoxy]ethoxy]ethoxy]ethoxy]ethoxy]ethyl 4-methylbenzenesulfonate (230 mg, 252.74 μmol, 1 equiv) in CH₃CN (5 mL) was added 5-[[[3-ethyl-5-[(2S)-2-(2-hydroxyethyl)-1-piperidyl]pyrazolo[1,5-a]pyrimidin-7-yl]amino]methyl]pyridin-2-ol (150 mg, 378.32 μmol, 1.50 equiv), K₂CO₃ (110 mg, 795.91 μmol, 3.15 equiv) and KI (50 mg, 301.20 μmol, 1.19 equiv). The mixture was stirred at 70° C. for 10 h. LCMS showed the starting material was consumed completely and the desired MS was detected. Solvent was removed to give a residue. It was purified by prep-HPLC (column: Unisil 3-100 C18 Ultra 150*50 mm*3 m; mobile phase: [water(FA)-ACN]; B%: 25%-55%, 10 min). Compound tert-butyl N-[(3R,4R)-1-[6-[[1-[2-[2-[2-[2-[2-[2-[2-[[5-[[[3-ethyl-5-[(2R)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethyl]-3-methoxy-pyrazol-4-yl]amino]-9-methyl-purin-2-yl]-4-fluoro-pyrrolidin-3-yl]carbamate (53 mg, 45.42 μmol, 17.97% yield, 97.2% purity) was obtained as a brown solid. ¹H NMR (400 MHz, CDCl₃) δ ppm 8.05 (d, H, J=2.4 Hz), 7.96 (s, 1H), 7.55 (s, 1H), 7.51 (dd, 1H, J=8.4, 2.4 Hz), 7.47 (s, 1H) 7.40-7.24(m, 1H), 6.71 (d, 1H, J=8.4 Hz), 6.30 (s, 1H), 5.22 (s, 1H) 5.20-5.05 (m, 1H), 5.03-4.91 (m, 2H,), 4.40-4.34 (m, 4H), 4.29 (d, 1H, J=3.2 Hz), 4.08 (t, 2H, J=5.2 Hz), 3.90-3.81 (m, 6H,), 3.77-3.73 (m, 4H), 3.62 (s, 5H), 3.60-3.43 (m, 23H), 3.30-3.24 (m, 1H), 3.00-2.93 (m, 1H), 2.57-2.50 (m, 2H), 2.01 (d, 1H, J=2.4 Hz), 1.66-1.60 (m, 4H), 1.46-1.35 (m, 11H), 1.17 (t, 3H, J=7.6 Hz).

Step 8. To a solution of tert-butyl N-[(3R, 4R)-1-[6-[[1-[2-[2-[2-[2-[2-[2-[2-[[5-[[[3-ethyl-5-[(2R)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethyl]-3-methoxy-pyrazol-4-yl]amino]-9-methyl-purin-2-yl]-4-fluoro-pyrrolidin-3-yl]carbamate (43 mg, 37.91 μmol, 1 equiv) in DCM (2 mL) was added TFA (616.00 mg, 5.40 mmol, 0.4 mL, 142.51 equiv). The mixture was stirred at 20° C. for 2 h. LCMS showed the starting material was consumed completely and the desired MS was detected. The crude product 2-[(2R)-1-[7-[[6-[2-[2-[2-[2-[2-[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy]ethoxy]ethoxy]ethoxy]ethoxy]ethoxy]-3-pyridyl]methylamino]-3-ethylpyrazolo[1,5-a]pyrimidin-5-yl]-2-piperidyl]ethanol (37 mg, 35.78 μmol, 94.38% yield) as a yellow oil was used into the next step without further purification. To a solution of 2-[(2R)-1-[7-[[6-[2-[2-[2-[2-[2-[2-[2-[4-[[2-[(3R,4R)-3-amino-4-fluoro- pyrrolidin-1-yl]-9-methyl-purin-6-yl]amino]-3-methoxy-pyrazol-1-yl]ethoxy]ethoxy]ethoxy]ethoxy]ethoxy]ethoxy]ethoxy]-3-pyridyl]methylamino]-3-ethylpyrazolo[ 1,5-a]pyrimidin-5-yl]-2-piperidyl]ethanol (37 mg, 35.78 μmol, 1 equiv) in THF (2 mL) was added DIPEA (14.84 mg, 114.82 μmol, 0.02 mL, 3.21 equiv) andprop-2-enoyl prop-2-enoate (0.1 M, 0.5 mL, 1.40 equiv). The mixture was stirred at 0° C. for 0.5 h. LCMS (showed the starting material was consumed completely and the desired MS was detected. Solvent was removed to give a residue. It was purified by prep-HPLC (column: Waters X bridge 150*25 mm*5 m; mobile phase: [water (NH₄HCO₃)-ACN]; B%: 37%-67%, 8 min). Compound N—[(3R,4R)-1-[6-[[1-[2-[2-[2-[2-[2-[2-[2-[[5-111[[[3-ethyl-5-[(2R)-2-(2-hydroxyethyl)-1-piperidyl]pyrazolo[ 1,5-a]pyrimidin-7-yl]amino]methyl]-2-pyridyl]oxy]ethoxy]ethoxy]ethoxy]ethoxy]ethoxy]ethoxy]ethyl]-3-methoxy-pyrazol-4-yl]amino]-9-methyl-purin-2-yl]-4-fluoro-pyrrolidin-3-yl]prop-2-enamide (15.98 mg, 14.68 μmol, 41.04% yield, 100% purity) was obtained as an off-white solid. ¹H NMR (400 MHz, CDCl₃) δ ppm 8.15 (s, 1H), 8.13 (d, 1H, J=2.45 Hz), 7.65 (s, 1H), 7.59 (dd, 1H, J=8.56, 2.57 Hz), 7.49 (s, 1H), 7.20 (s, 1H), 6.91 (dd, 1H, J=5.20, 1.77 Hz), 6.79 (d, 1H, J=8.44 Hz), 6.40-6.34 (m, 1H), 6.32 (t, 1H, J=5.50 Hz), 6.25-6.17 (m, 1H), 5.64 (dd, 1H, J=10.15, 1.71 Hz), 5.35-5.21 (m, 2H), 5.14-5.08 (m, 1H), 4.73-4.66 (m, 1H), 4.47 (dd, 2H, J=5.44, 4.10 Hz), 4.43 (d, 2H, J=5.50 Hz), 4.20-4.15 (m, 2H), 4.03-4.08 (m, 1H), 3.98-3.91 (m, 5H), 3.84 (dd, 2H, J=5.32, 4.22 Hz), 3.79 (t, 2H, J=4.95 Hz), 3.72-3.67 (m, 6H), 3.65-3.49 (m, 18H), 3.42-3.32 (m, 5H), 3.09-3.01 (m, 1H,), 2.62 (qd, 2H, J=7.48, 2.75 Hz), 2.13-2.06 (m, 1H), 1.89-1.71 (m, 6H), 1.27 (t, 3H, J=7.52 Hz). LC-MS: MS (ES⁺): RT=2.324 min, m/z=443.0 [M+H⁺].

### Example 125—Synthesis of Compound I-141

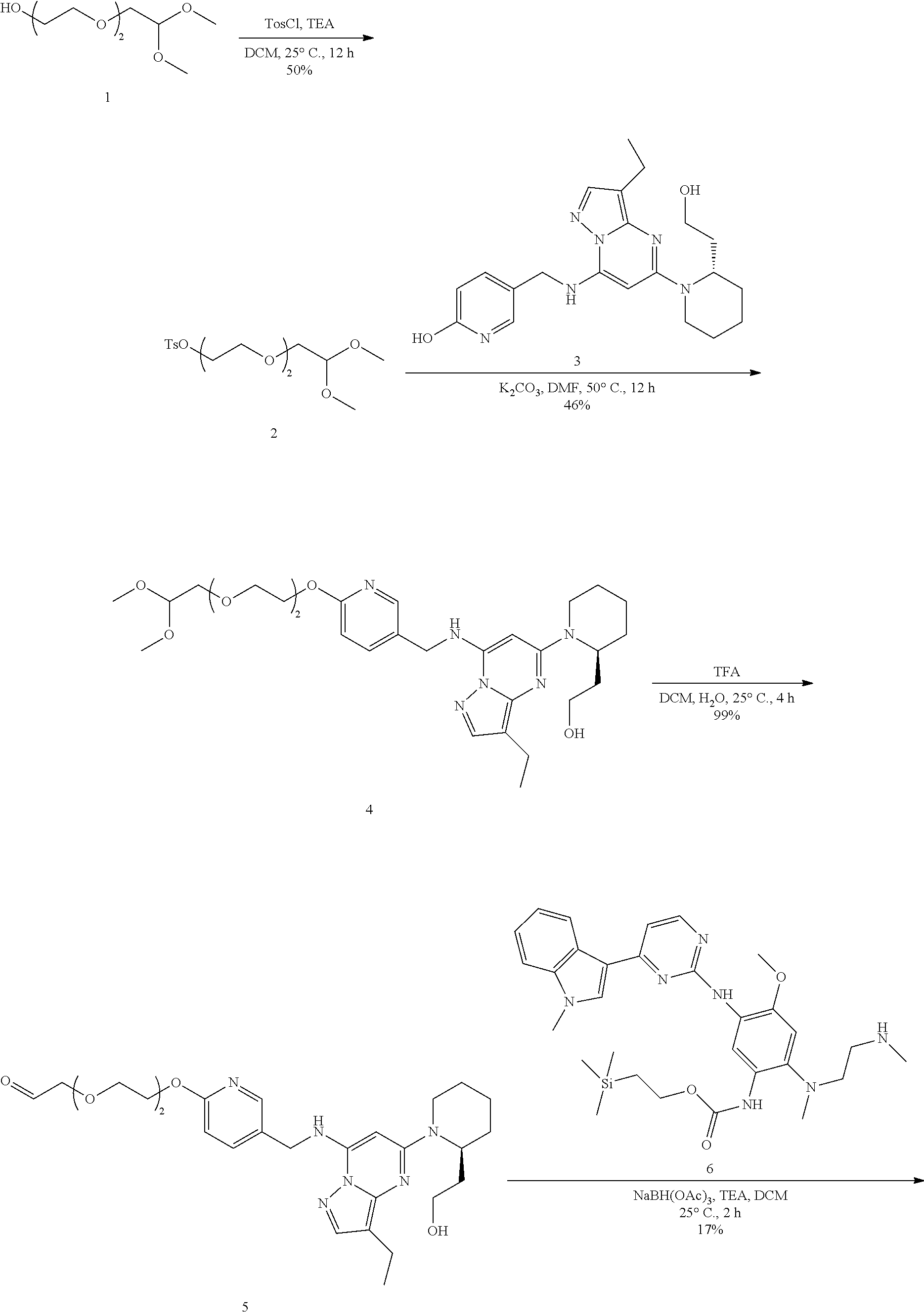

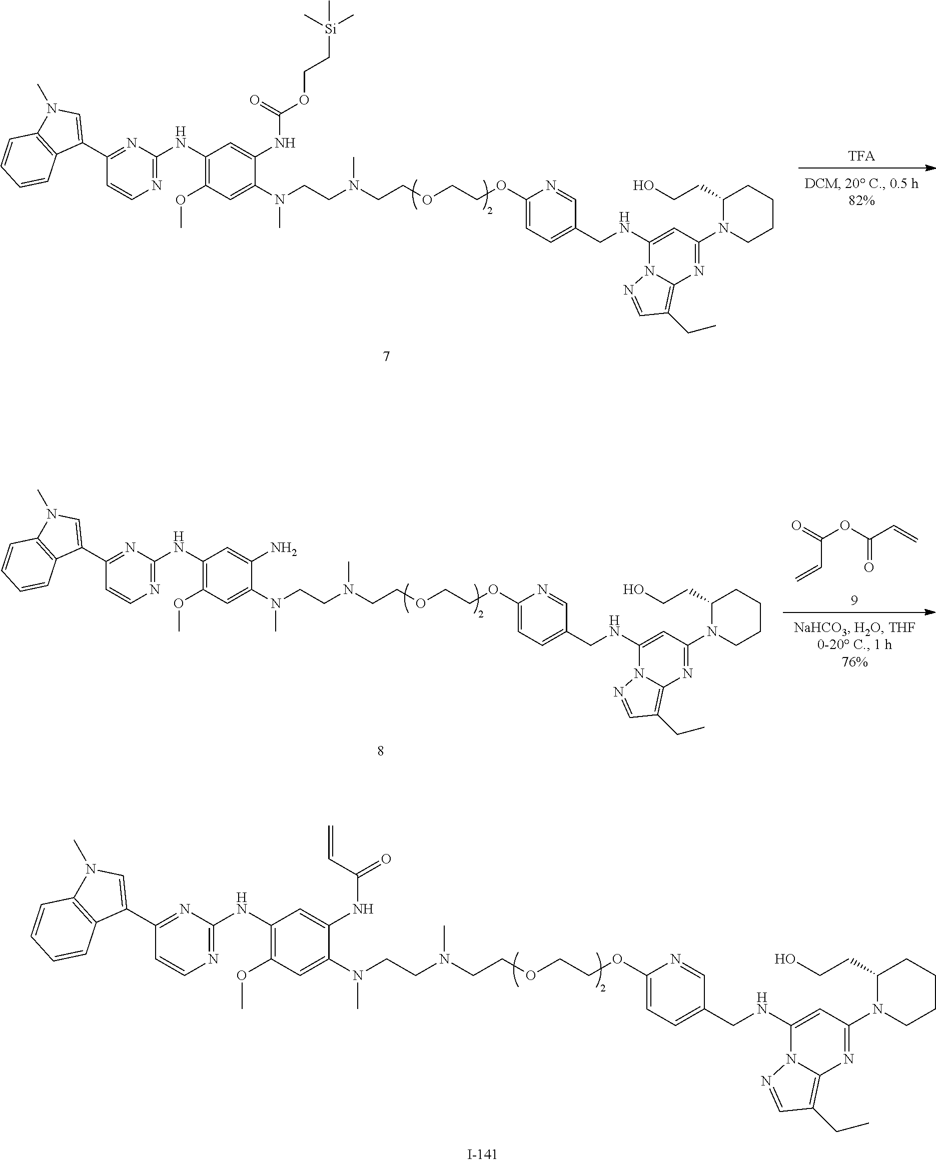

Step 1. To a solution of 2-[2-(2,2-dimethoxyethoxy)ethoxy]ethanol (1 g, 5.15 mmol, 1.0 equiv) in DCM (40 mL) was added TEA (1.56 g, 15.45 mmol, 2.15 mL, 3.0 equiv) and TosCl (726 mg, 10.30 mmol, 2.0 equiv). The mixture was stirred at 25° C. for 12 h. The reaction mixture was diluted with 50 mL water and extracted with DCM (30 mL×3). The combined organic layers were washed with brine (30 mL×3), dried over Na₂SO₄, filtered and concentrated under reduced pressure to give a residue. The residue was purified by column chromatography (SiO₂, Petroleum ether/Ethyl acetate=50/1 to 2/1) to give 2-[2-(2,2-dimethoxyethoxy)ethoxy]ethyl 4-methylbenzenesulfonate (900 mg, 50% yield) as a white solid.

Step 2. To a solution of 2-[2-(2,2-dimethoxyethoxy)ethoxy]ethyl 4-methylbenzenesulfonate (593 mg, 1.70 mmol, 1.5 equiv) in DMF (7 mL) and 5-[[[3-ethyl-5-[(2S)-2-(2-hydroxyethyl)-1-piperidyl]pyrazolo[ 1,5-a]pyrimidin-7-yl]amino]methyl]pyridin-2-ol (450 mg, 1.13 mmol, 1.0 equiv) was added K₂CO₃ (470 mg, 3.40 mmol, 3.0 equiv). The mixture was stirred at 50° C. for 12 h. After filtered, the residue was purified by prep-HPLC (column: Waters Xbridge 150*25 mm*5 m; mobile phase: [water(NH₄HCO₃)-ACN]; B%: 47%-77%, 10 min) to give 2-[(2S)-1-[7-[[6-[2-[2-(2,2-dimethoxyethoxy)ethoxy]ethoxy]-3-pyridyl]methylamino]-3-ethyl-pyrazolo[1,5-a]pyrimidin-5-yl]-2-piperidyl]ethanol (400 mg, 61% yield) as a colorless oil.

Step 3. To a solution of 2-[(2S)-1-[7-[[6-[2-[2-(2,2-dimethoxyethoxy)ethoxy]ethoxy]-3-pyridyl]methylamino]-3-ethyl-pyrazolo[1,5-a]pyrimidin-5-yl]-2-piperidyl]ethanol (230 mg, 401 μmol, 1.0 equiv) in DCM (10 mL) was added TFA (1 mL) and H₂O (200 mg, 0.2 mL). The mixture was stirred at 25° C. for 4 h. The mixture used for the next step as a DCM solution.

Step 4. To a solution of 2-trimethylsilylethyl N-[4-methoxy-5-[[4-(1-methylindol-3-yl)pyrimidin-2-yl]amino]-2-[methyl-[2-(methylamino)ethyl]amino]phenyl]carbamate (251 mg, 436 μmol, 1.0 equiv)in DCM (10 mL) was added NaBH(OAc)₃ (1.39 g, 6.55 mmol, 15.0 equiv), TEA (441 mg, 4.37 mmol, 607 μL, 10.0 equiv), and 2-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]acetaldehyde (230 mg, 436 μmol, 1.0 equiv) in DCM (1OmL) at 0° C. The mixture was stirred at 25° C. for 2 h. The reaction mixture was diluted with 20 mL water and extracted with DCM (10 mL×3). The combined organic layers were washed with brine (10 mL×3), dried over Na₂SO₄, filtered and concentrated under reduced pressure to give a residue. The residue was purified by prep-HPLC (column: 3_Phenomenex Luna C18 75*30 mm*3 m; mobile phase: [water(TFA)-ACN]; B%: 35%-55%, 7 min) to give 2-trimethylsilylethyl N-[2-[2-[2-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethyl-methyl-amino]ethyl-methyl-amino]-4-methoxy-5-[[4-(1-methylindol-3-yl)pyrimidin-2-yl]amino]phenyl]carbamate (80 mg, 17% yield) as a colorless solid.

Step 5. To a solution of 2-trimethylsilylethyl N-[2-[2-[2-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethyl-methyl-amino]ethyl-methyl-amino]-4-methoxy-5-[[4-(1-methylindol-3-yl)pyrimidin-2-yl]amino]phenyl]carbamate (70 mg, 64 μmol, 1.0 equiv) in DCM (2 mL) was added TFA (0.5 mL), and then it was stirred at 20° C. for 0.5 h. The reaction mixture was concentrated to afford crude product.

Step 6. To a solution of 2-[(2S)-1-[7-[[6-[2-[2-[2-[2-[2-amino-5-methoxy-N-methyl-4-[[4-(1-methylindol-3-yl)pyrimidin-2-yl]amino]anilino]ethyl-methyl-amino]ethoxy]ethoxy]ethoxy]-3-pyridyl]methylamino]-3-ethyl-pyrazolo[1,5-a]pyrimidin-5-yl]-2-piperidyl]ethanol (50 mg, 53 μmol, 1.0 equiv) in H₂O (0.5 mL) and THF (0.2 mL) was added NaHCO₃ (40 mg, 530 μmol, 10.0 equiv), The mixture was added prop-2-enoyl prop-2-enoate (6 mg, 53 μmol, 1.0 equiv) in THF (0.3 mL) at 0° C. The mixture was stirred at 20° C. for 1 h. After concentrated, the residue was purified by prep-HPLC (column: Unisil 3-100 C18 Ultra 150*50 mm*3 m; mobile phase: [water(FA)-ACN]; B%: 10%-40%, 10 min) to give N-[2-[2-[2-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethyl-methyl-amino]ethyl-methyl-amino]-4-methoxy-5-[[4-(1-methylindol-3-yl)pyrimidin-2-yl]amino]phenyl]prop-2-enamide (40 mg, 76% yield) was obtained as a white solid. ¹H NMR (400 MHz, CD₃OD) 6 9.20 (s, 1H), 8.46 (d, J=7.88, 6.00 Hz, 1H), 8.24 (d, J=5.60 Hz, 1H), 8.20 (d, J=8.08 Hz, 1H), 8.06 (d, J=2.08 Hz, 1H), 7.58-7.65 (m, 2H), 7.40 (d, J=8.08 Hz, 1H), 7.14-7.24 (m, 3H), 6.94 (s, 1H), 6.55-6.65 (m, 2H), 6.37-6.45 (m, 1H), 5.80 (d, J=12.00 Hz, 1H), 5.44 (s, 1H), 4.59 (s, 1H), 4.41 (s, 2H), 4.20-4.26 (m, 2H), 3.93 (s, 3H), 3.89 (s, 3H), 3.63-3.67 (m, 2H), 3.57 (d, J=4.40 Hz, 2H), 3.48-3.53 (m, 3H), 3.43-3.47 (m, 2H), 3.37 (d, J=10.24, 3.60 Hz, 1H), 3.05-3.20 (m, 2H), 2.68 (s, 3H), 2.53-2.58 (m, 2H), 2.46-2.52 (m, 2H), 1.97-2.22 (m, 2H), 1.59-1.69 (m, 6H), 1.37-1.50 (m, 2H), 1.29 (s, 6H), 1.22 (t, J=7.56 Hz, 3H), 0.84-0.93 (m, 2H). LC-MS: MS (ES′): RT=0.768 min, m/z=996.5 [M+H⁺]; LCMS Method: 25.

### Example 126—Synthesis of Compound I-142

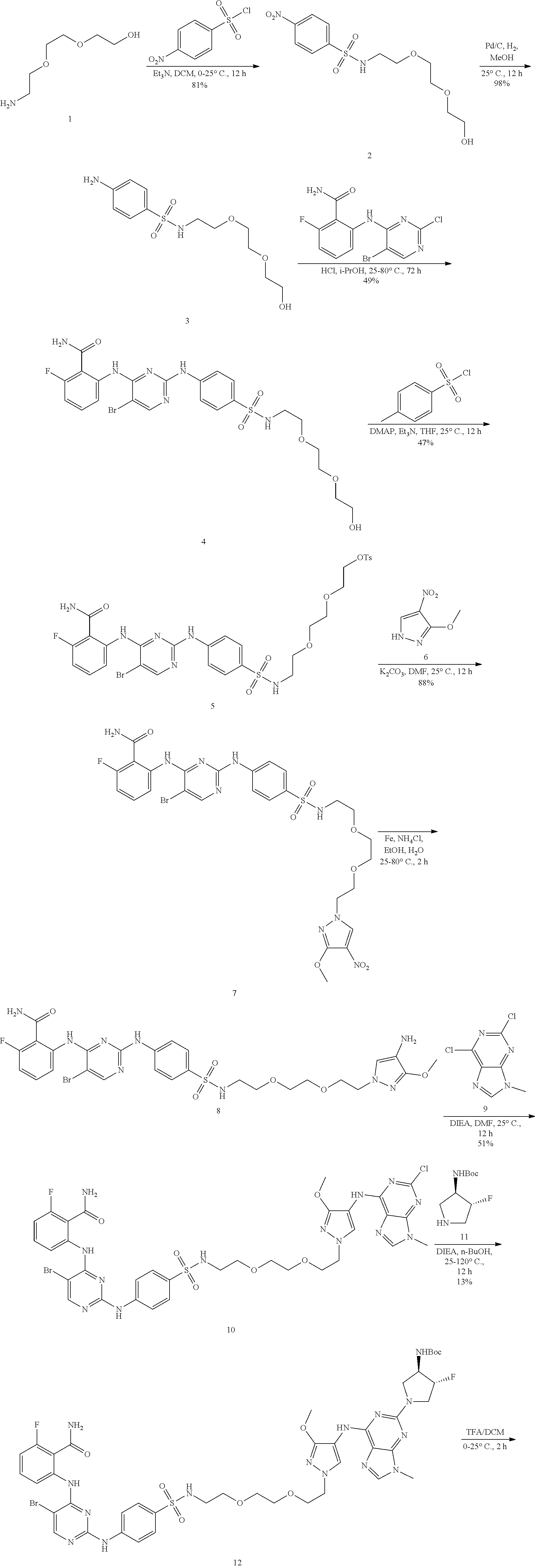

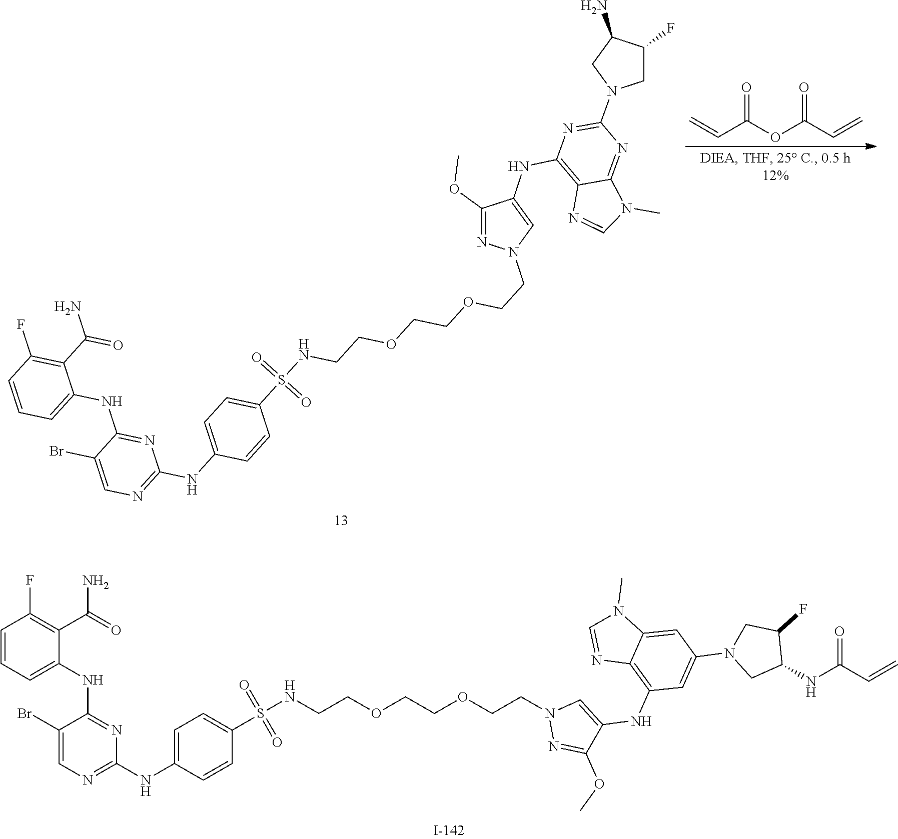

Step 1. To a solution of 2-[2-(2-aminoethoxy)ethoxy]ethanol (3.3 g, 22.12 mmol, 1.2 equiv) and Et₃N (2.91 g, 28.74 mmol, 4 mL, 1.5 equiv) in DCM (100 mL) was added 4-nitrobenzenesulfonyl chloride (4 g, 18.05 mmol, 1 equiv) at 0° C. Then the mixture was stirred at 25° C. for 12 hr. The reaction solution was concentrated to give the residue. The residue was purified by flash silica gel chromatography (ISCO®; 40 g SepaFlash® Silica Flash Column, Eluent of 0-60%Ethylacetate/Petroleum ether gradient at 50 mL/min). Compound N-[2-[2-(2-hydroxy ethoxy)ethoxy]ethyl]-4-nitro-benzenesulfonamide (4.9 g, 14.66 mmol, 81% yield) was obtained as a light yellow solid. ¹H NMR (400 MHz, CDCl₃) δ8.42-8.24 (m, 2H), 8.16-8.01 (m, 2H), 6.67 (br t, J=5.2 Hz, 1H), 3.80 (br d, J=4.0 Hz, 2H), 3.67-3.61 (m, 4H), 3.60-3.52 (m, 4H), 3.29 (br s, 1H), 3.18 (q, J=5.2 Hz, 2H).

Step 2. To a solution of N-[2-[2-(2-hydroxyethoxy)ethoxy]ethyl]-4-nitro-benzene sulfonamide (4.9 g, 14.66 mmol, 1 equiv) in MeOH (50 mL) was added Pd/C (300 mg, 10% purity) under N₂. The suspension was degassed under vacuum and purged with H₂ several times. The mixture was stirred under H₂ (50 psi) at 25° C. for 12 h. The reaction mixture was filtered and the filter was concentrated to give the product. Compound 4-amino-N-[2-[2-(2-hydroxyethoxy) ethoxy]ethyl]benzenesulfonamide (4.4 g, 14.46 mmol, 98% yield) was obtained as a yellow oil. ¹H NMR (400 MHz, CDCl₃) δ7.68-7.55 (m, 2H), 6.72-6.61 (m, 2H), 4.24 (s, 2H), 3.81-3.71 (m, 2H), 3.64-3.54 (m, 6H), 3.54-3.49 (m, 2H), 3.08 (t, J=5.2 Hz, 2H).

Step 3. To a solution of 4-amino-N-[2-[2-(2-hydroxyethoxy)ethoxy]ethyl]benzenesulfonamide (4.4 g, 14.59 mmol, 1.2 equiv) and HCl (12 M, 1 mL, 1.0 equiv) in i-PrOH (100 mL) was added 2-[(5-bromo-2-chloro-pyrimidin-4-yl)amino]-6-fluoro-benzamide (4 g, 11.58 mmol, 1.0 equiv) at 25° C. The mixture was stirred at 80° C. for 72 hours. The reaction mixture was concentrated to give a residue. The residue was purified by prep-HPLC (column: Kromasil Eternity XT 250*80 mm*10 m; mobile phase: [water (ammonia hydroxide v/v)-ACN]; B%: 25%-55%, 20 min) to give 2-[[5-bromo-2-[4-[2-[2-(2-hydroxyethoxy)ethoxy]ethylsulfamoyl]anilino]pyrimidin-4-yl]amino]-6-fluoro-benzamide (3.5 g, 10.48 mmol, 49% yield) was obtained as a white solid. ¹H NMR (400 MHz, DMSO-d₆) δ 10.38-10.14 (m, 2H), 8.43 (s, 1H), 8.14 (br d, J=14.8 Hz, 3H), 7.79 (br d, J=8.8 Hz, 2H), 7.65 (d, J=8.8 Hz, 2H), 7.62-7.43 (m, 2H,), 7.13 (t, J=9.2 Hz, 1H), 3.55-3.41 (m, 6H), 3.38 (t, J=5.2 Hz, 4H), 2.83-2.82 (m, 1H), 2.86 (br t, J=5.6 Hz, 1H).

Step 4. To a solution of 2-[[5-bromo-2-[4-[2-[2-(2-hydroxyethoxy)ethoxy]ethyl sulfamoyl]anilino]pyrimidin-4-yl]amino]-6-fluorobenzamide (2.7 g, 4.40 mmol, 1.0 equiv) and Et₃N (1.34 g, 13.20 mmol, 1.84 mL, 3.0 equiv) in THF (100 mL) was added DMAP (54 mg, 442 μmol, 0.1 equiv) at 25° C. Then the 4-methylbenzenesulfonyl chloride (1.7 g, 8.92 mmol, 2.0 equiv) was added to the mixture solution. The mixture was stirred at 25° C. for 12 h. The reaction mixture was concentrated under reduced pressure to remove the solvent. The residue was diluted with H₂O (100 mL) and extracted with DCM (100 mL*3). The combined organic layers were washed with brine (300 mL), dried over Na₂SO₄, filtered and concentrated under reduced pressure to give a residue. Compound 2-[2-[2-[[4-[[5-bromo-4-(2-carbamoyl-3-fluoro-anilino)pyrimidin-2-yl]amino]phenyl]sulfonylamino]ethoxy]ethoxy]ethyl 4-methylbenzenesulfonate (1.6 g, 2.08 mmol, 47% yield) was obtained as a white solid, and used for next step directly.

Step 5. To a solution of 2-[2-[2-[[4-[[5-bromo-4-(2-carbamoyl-3-fluoro-anilino)pyrimidin-2-yl]amino]phenyl]sulfonylamino]ethoxy]ethoxy]ethyl-4-methylbenzene sulfonate (2 g, 2.61 mmol, 1.0 equiv) and K₂CO₃ (540 mg, 3.91 mmol, 1.5 equiv) in DMF (20 mL) was added 3-methoxy-4-nitro-1H-pyrazole (410 mg, 2.87 mmol, 1.1 equiv) at 25° C. Then the mixture was stirred at 25° C. for 12 hours. The reaction mixture was filtered and the filter was concentrated to give a residue. The residue was purified by prep-HPLC (FA condition, column: Phenomenex Synergi Max-RP 250*50 mm*10 m; mobile phase:[water(FA)-ACN]; B%: 35%-65%, 20 min). Compound 2-[[5-bromo-2-[4-[2-[2-[2-(3-methoxy-4-nitro-pyrazol-1-yl) ethoxy]ethoxy]ethylsulfamoyl]anilino]pyrimidin-4-yl]amino]-6-fluoro-benzamide (1.7 g, 2.30 mmol, 88% yield) was obtained as a white solid. ¹H NMR (400 MHz, DMSO-d₆) δ10.11 (s, 1H), 9.93 (s, 1H), 8.63 (s, 1H), 8.38 (s, 1H), 8.23 (br d, J=8.4 Hz, 1H), 8.19-8.08 (m, 2H), 7.84 (d, J=8.8 Hz, 2H), 7.63 (d, J=8.8 Hz, 2H), 7.56-7.45 (m, 2H), 7.09 (t, J=9.2 Hz, 1H), 4.15 (t, J=5.2 Hz, 2H), 3.91 (s, 3H), 3.74 (t, J=5.2 Hz, 2H), 3.50-3.45 (m, 2H), 3.43-3.39 (m, 2H), 3.37-3.34 (m, 2H), 2.85 (q, J=6.0 Hz, 2H).

Step 6. To a solution of 2-[[5-bromo-2-[4-[2-[2-[2-(3-methoxy-4-nitro-pyrazol-1-yl)ethoxy]ethoxy]ethylsulfamoyl]anilino]pyrimidin-4-yl]amino]-6-fluoro-benzamide (1.7 μg, 2.30 mmol, 1.0 equiv) and Fe (1.29 g, 23.02 mmol, 10.0 equiv) in EtOH (150 mL) and H₂O (10 mL) was added NH₄Cl (1.23 g, 23.02 mmol, 10.0 equiv) at 25° C. Then the mixture was stirred at 80° C. for 2 h. The reaction mixture was concentrated under reduced pressure to remove EtOH. The residue was diluted with H₂O (100 mE), and extracted with EtOAc (100 mL×3). The combined organic layers were washed with brine mL (500 mL), dried over Na₂SO₄, filtered and concentrated under reduced pressure to give a crude product. Compound 2-[[2-[4-[2-[2-[2-(4-amino-3-methoxy-pyrazol-1-yl)ethoxy]ethoxy]ethylsulfamoyl]anilino]-5-bromo-pyrimidin-4-yl]amino]-6-fluoro-benzamide.

Step 7. To a solution of 2-[[2-[4-[2-[2-[2-(4-amino-3-methoxy-pyrazol-1-yl)ethoxy]ethoxy]ethylsulfamoyl]anilino]-5-bromo-pyrimidin-4-yl]amino]-6-fluoro-benzamide (1.9 g, 2.68 mmol, 1.0 equiv) and DIEA (693 mg, 5.36 mmol, 934 μL, 2.0 equiv) in DMF (20 mL) was added 2,6-dichloro-9-methyl-purine (811 mg, 4.00 mmol, 1.5 equiv) at 25° C. Then the mixture was stirred at 25° C. for 12 h. The residue was diluted with H₂O (100 mL), and extracted with EtOAc (70 mL×3). The combined organic layers were washed with brine (300 mL), dried over Na₂SO₄, filtered and concentrated under reduced pressure to give a residue. The residue was purified by prep-HPLC (column: Phenomenex luna C18 (250*70 mm*10 μm); mobile phase: [water(FA)-ACN]; B%: 30%-60%, 21 min). Compound 2-[[5-bromo-2-[4-[2-[2-[2-[4-[(2-chloro-9-methyl-purin-6-yl)amino]-3-methoxy-pyrazol-1-yl]ethoxy]ethoxy]ethylsulfamoyl]anilino]pyrimidin-4-yl]amino]-6-fluoro-benzamide (1.2 g, 1.37 mmol, 51% yield) was obtained as a gray solid. ¹H NMR (400 MHz, DMSO-d₆) δ10.11 (br s, 1H), 9.92 (s, 1H), 9.43-8.82 (m, 1H), 8.38 (s, 1H), 8.32-7.99 (m, 4H), 7.83 (d, J=8.8 Hz, 2H), 7.69 (br s, 1H), 7.62 (d, J=8.8 Hz, 2H), 7.57-7.42 (m, 2H), 7.08 (t, J=9.2 Hz, 1H), 4.07 (br t, J=5.2 Hz, 2H), 3.77 (br s, 3H), 3.73-3.69 (m, 4H), 3.50-3.46 (m, 2H), 3.45-3.42 (m, 2H), 3.41-3.38 (m, 3H), 2.85 (br d, J=3.2 Hz, 2H).

Step 8. To a solution of 2-[[5-bromo-2-[4-[2-[2-[2-[4-[(2-chloro-9-methyl-purin-6-yl)amino]-3-methoxy-pyrazol-1-yl]ethoxy]ethoxy]ethylsulfamoyl]anilino]pyrimidin-4-yl]amino]-6-fluoro-benzamide (150 mg, 171 μmol, 1.0 equiv) and DIEA (45 mg, 348 μmol, 60 L, 2.0 equiv) in n-BuOH (2 mL) was added tert-butyl N-[(3R,4R)-4-fluoropyrrolidin-3-yl]carbamate (42 mg, 205 μmol, 1.2 equiv) at 25° C. Then the mixture was stirred at 120° C. for 12 h. The residue was concentrated under reduced pressure to give a residue. The residue was purified by prep-HPLC (column: Waters Xbridge 150*25 mm*5 m; mobile phase: [water (ammonia hydroxidev/v)-ACN]; B%: 30%-60%, 9 min). Compound tert-butyl N-[(3R,4R)-1-[6-[[1-[1[2-[2-[[4-[[5-bromo-4-(2-carbamoyl-3-fluoro-anilino)pyrimidin-2-yl]amino]phenyl]sulfonylamino]ethoxy]ethoxy]ethyl]-3-methoxy-pyrazol-4-yl]amino]-9-methyl-purin-2-yl]-4-fluoro-pyrrolidin-3-yl]carbamate (25 mg, 23.97 μmol, 13% yield) was obtained as a yellow solid. ¹H NMR (400 MHz, DMSO-d₆) δ10.11 (br s, 1H), 9.92 (br s, 1H), 8.38 (s, 1H), 8.23 (br d, J=8.0 Hz, 1H), 8.13 (br d, J=18.8 Hz, 2H), 7.98 (br s, 1H), 7.91-7.80 (m, 3H), 7.77 (1H, br s), 7.62 (br d, J=8.8 Hz, 2H), 7.56-7.42 (m, 2H), 7.39-7.28 (m, 1H), 7.13-7.02 (m, 1H), 5.18-4.95 (m, 1H), 4.19-4.03 (m, 3H), 3.86-3.67 (m, 8H), 3.64-3.40 (m, 10H), 2.83 (br d, J=1.2 Hz, 10H), 1.37 (9H, br s).

Step 9. To a solution of tert-butyl N-[(3R,4R)-1-[6-[[1-[2-[2-[2-[[4-[[5-bromo-4-(2-carbamoyl-3-fluoro-anilino)pyrimidin-2-yl]amino]phenyl]sulfonylamino]ethoxy]ethoxy]ethyl]-3-methoxy-pyrazol-4-yl]amino]-9-methyl-purin-2-yl]-4-fluoro-pyrrolidin-3-yl]carbamate (25 mg, 23 μmol, 1.0 equiv) in DCM (8 mL) was added TFA (2 mL) at 0° C. Then the mixture was stirred at 0° C. for 2 h. The residue was concentrated under reduced pressure to give a residue. Compound 2-[[2-[4-[2[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy]ethylsulfamoyl]anilino]-5-bromo-pyrimidin-4-yl]amino]-6-fluoro-benzamide (23 mg, crude) was obtained as a yellow oil, and used for next step directly.

Step 10. To a solution of 2-[[2-[4-[2-[2-[2-[4-[[2-[(3R,4R)-3-amino-4-fluoro-pyrrolidin-1-yl]-9-methyl-purin-6-yl]amino]-3-methoxypyrazol-1-yl]ethoxy]ethoxy]ethylsulfamoyl]anilino]-5-bromo-pyrimidin-4-yl]amino]-6-fluoro-benzamide (23 mg, 24 μmol, 1.0 equiv) and DIEA (9.46 mg, 73 μmol, 12 μL, 3.0 equiv) in THF (3 mL) was added prop-2-enoyl prop-2-enoate (5 mg, 39 μmol, 1.6 equiv) in THF (1 mL) at 25° C. Then the mixture was stirred at 25° C. for 0.5 hours. The residue was concentrated under reduced pressure to give a residue. The residue was purified by prep-HPLC (column: Phenomenex C18 75*30 mm*3 m; mobile phase: [water(FA)-ACN]; B%: 30%-60%, 7 min). Compound 2-[[5-bromo-2-[4-[2-[2-[2-[4-[[2-1[(3R,4R)-3-fluoro-4-(prop-2-enoylamino)pyrrolidin-1-yl]-9-methyl-purin-6-yl]amino]-3-methoxy-pyrazol-1-yl]ethoxy]ethoxy]ethylsulfamoyl]anilino]pyrimidin-4-yl]amino]-6-fluoro-benzamide (3.11 mg, 3.00 μmol, 12% yield, 96.3% purity) was obtained as an off-white solid. ¹H NMR (400 MHz, DMSO-d₆) δ10.11 (s, 1H), 9.92 (s, 1H), 8.46 (d, J=6.8 Hz, 1H), 8.39-8.36 (m, 1H), 8.23 (br d, J=8.4 Hz, 1H), 8.14 (br d, J=17.6 Hz, 2H), 8.04 (s, 1H), 7.91-7.75 (m, 4H), 7.62 (d, J=8.8 Hz, 2H), 7.54-7.44 (m, 2H), 7.08 (t, J=9.2 Hz, 1H), 6.30-6.04 (m, 2H), 5.66-5.55 (m, 1H), 5.22-4.98 (m, 1H), 4.50-4.39 (m, 1H), 4.08 (t, J=5.2 Hz, 2H), 3.81 (s, 4H), 3.80-3.62 (m, 5H), 3.61 (s, 3H), 3.49-3.44 (m, 5H), 2.82 (q, J=5.6 Hz, 2H). LC-MS: MS (ES⁺): RT=2.340 min, m/z=998.1 [M+H⁺]; LCMS Method: 10.

### Example 127—Synthesis of Compound I-143

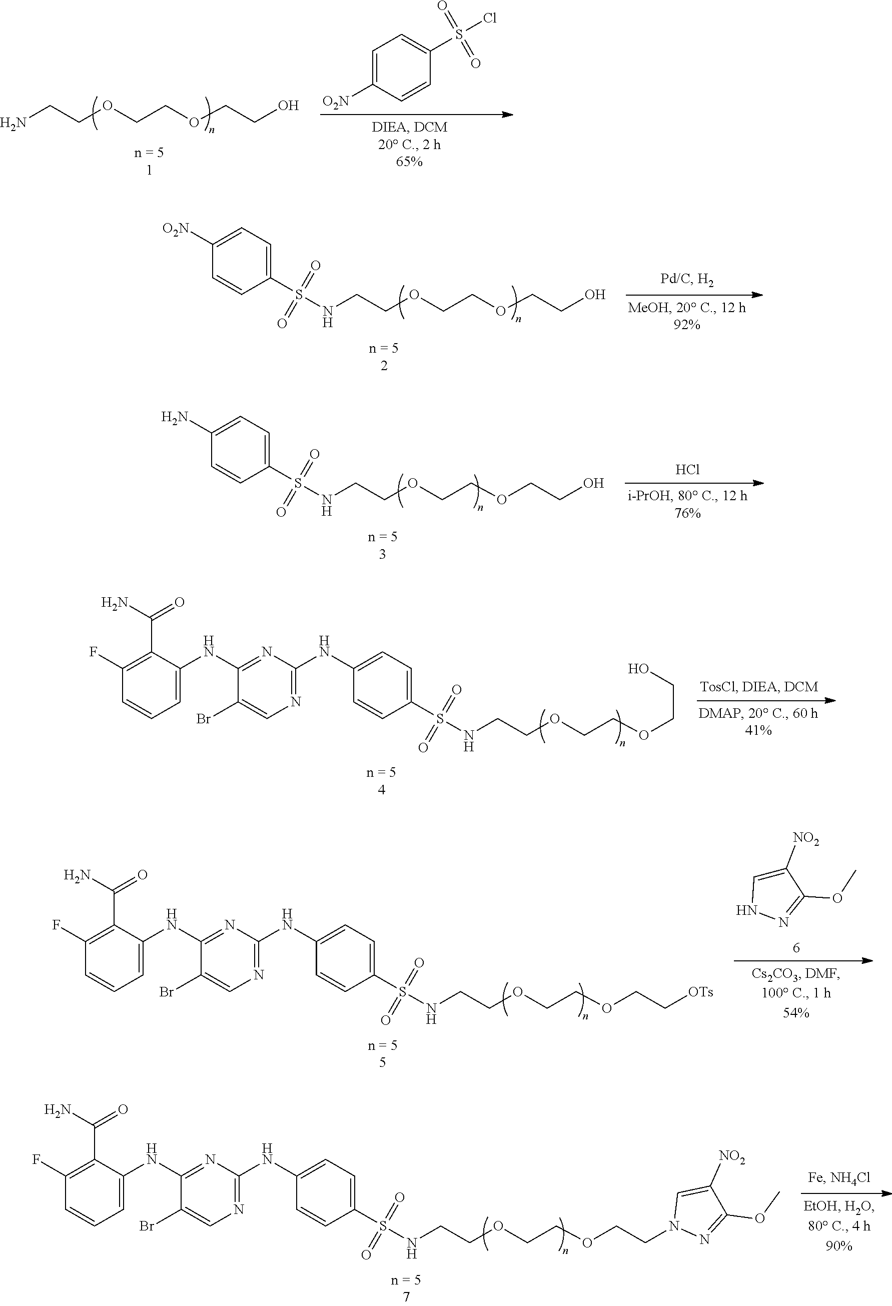

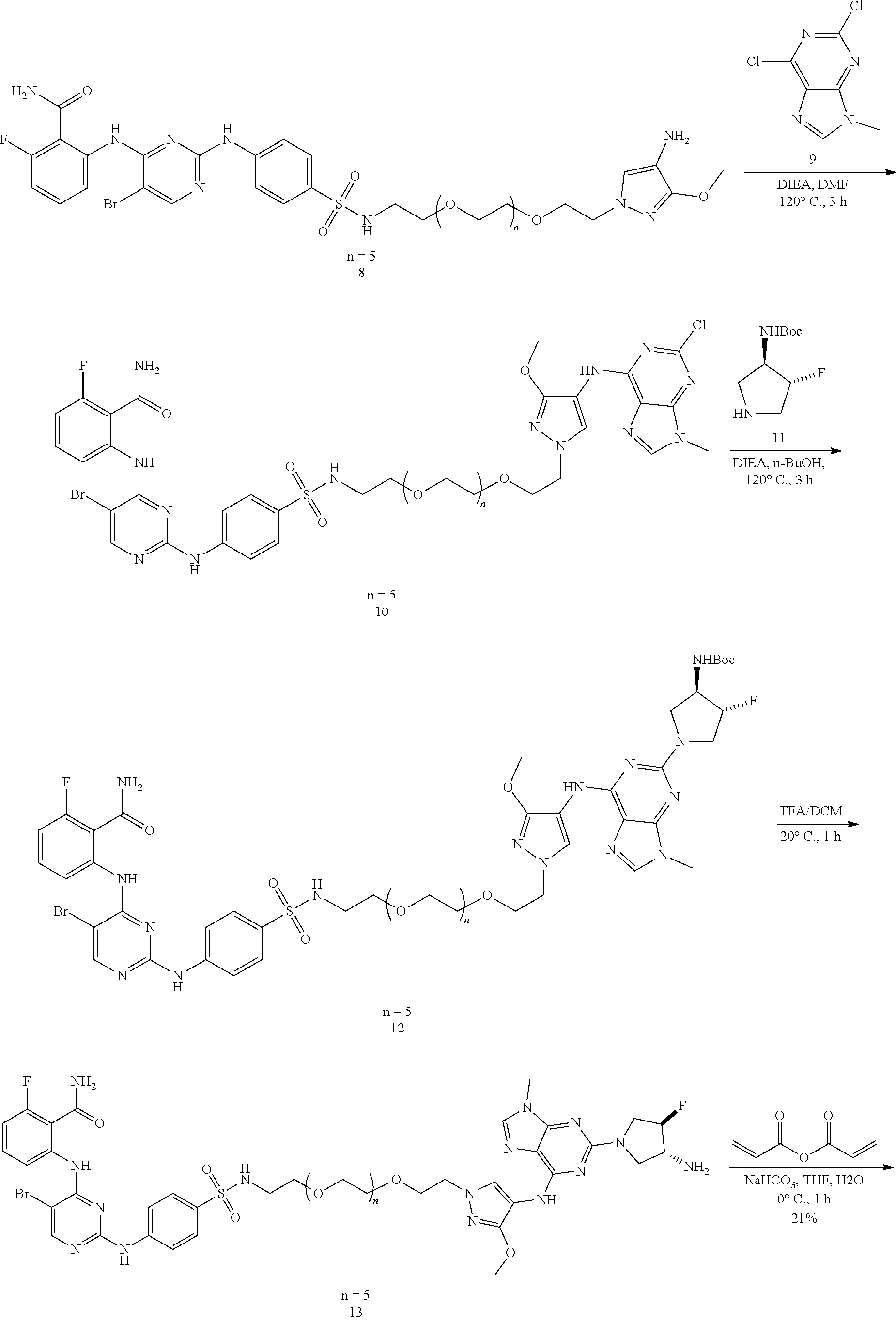

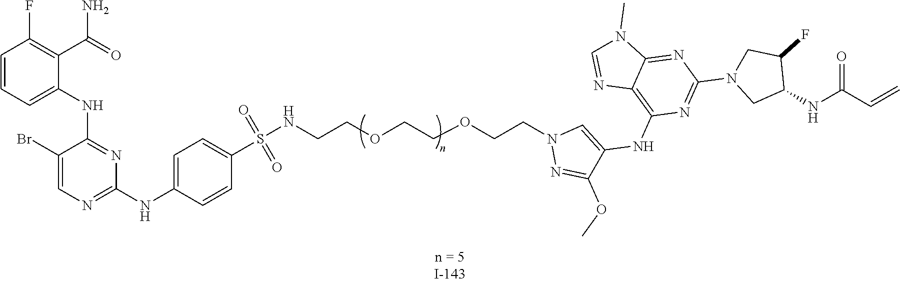

Step 1. To a solution of 2-[2-[2-[2-[2-[2-(2-aminoethoxy)ethoxy]ethoxy]ethoxy]ethoxy]ethoxy]ethanol (3 g, 9.22 mmol, 1 equiv) and DIEA (2.38 g, 18.44 mmol, 3.21 mL, 2 equiv) in DCM (40 mL) was added 4-nitrobenzenesulfonyl chloride (2.15 g, 9.68 mmol, 1.05 equiv). The mixture was stirred at 20° C. for 2 h. The mixture was evaporated. The residue was purified by column chromatography on silica gel (petroleum ether: ethyl acetate=1:1 to 0:1) to give the N-[22[2-[2-[2-[2-(2-hydroxyethoxy)ethoxy]ethoxy]ethoxy]ethoxy]ethoxy]ethyl]-4-nitro-benzenesulfonamide (3.1 g, 6.07 mmol, 65.86% yield) as a light yellow gum. ¹H NMR (400 MHz, DMSO-d₆) δ 8.44-8.39 (m, 2H), 8.16-8.10 (m, 1H), 8.08-8.03 (m, 2H), 4.59-4.54 (m, 1H), 3.51-3.46 (m, 17H), 3.43-3.35 (m, 9H), 3.03-2.95 (m, 2H).

Step 2. To a solution of Pd/C (0.3 g, 10% purity) in MeOH (40 mL) was added N-[2-[2-[2-[2-[2-[2-(2-hydroxyethoxy)ethoxy]ethoxy]ethoxy]ethoxy]ethoxy]ethyl]-4-nitro-benzenesulfonamide (3.1 g, 6.07 mmol, 1 equiv) under N₂. The suspension was degassed under vacuum and purged with H₂ several times. The mixture was stirred under H₂ (15 psi) at 20° C. for 12 h. The reaction mixture was filtered. The filtrate was concentrated. 4-amino-N-[2-[2-[2-[2-1[[2-(2-hydroxyethoxy)ethoxy]ethoxy]ethoxy]ethoxy]ethoxy]ethyl]benzenesulfonamide (2.7 g, 5.62 mmol, 92.53% yield) was obtained as a light yellow gum. ¹H NMR (400 MHz, DMSO-d₆) δ7.45-7.37 (m, 2H), 7.29-6.97 (m, 1H), 6.64-6.57 (m, 2H), 5.90 (s, 2H), 4.71-4.43 (m, 1H), 3.50-3.39 (m, 26H), 2.82-2.75 (m, 2H).

Step 3. To a solution of 2-[(5-bromo-2-chloro-pyrimidin-4-yl)amino]-6-fluoro-benzamide (1.6 g, 4.63 mmol, 1 equiv) and 4-amino-N-[2-[2-[2-[2-[2-[2-(2-hydroxyethoxy) ethoxy]ethoxy]ethoxy]ethoxy]ethoxy]ethyl]benzenesulfonamide (2.67 g, 5.56 mmol, 1.2 equiv) in i-PrOH (50 mL) was added HCl (12 M, 0.8 mL, 2.07 equiv). The mixture was stirred at 80° C. for 12 h. The mixture was evaporated. The residue was purified by prep-HPLC (column: Phenomenex Synergi Max-RP 250*50 mm*10 m; mobile phase: [water(FA)-ACN]; B%: 20%-50%, 21 min) to give the 2-[ [5-bromo-2-[4-[2-[2-[2-[2-[2-[2-(2-hydroxyethoxy)ethoxy]ethoxy]ethoxy]ethoxy]ethoxy]ethylsulfamoyl]anilino]pyrimidin-4-yl]amino]-6-fluoro-benzamide (2.8 g, 3.55 mmol, 76.58% yield) as a white solid. ¹H NMR (400 MHz, DMSO-d₆) δ10.11 (s, 1H), 9.93 (s, 1H), 8.39 (s, 1H), 8.24-8.20 (m, 1H), 8.18-8.09 (m, 2H), 7.89-7.80 (m, 2H), 7.68-7.60 (m, 2H), 7.56-7.46 (m, 2H), 7.14-7.04 (m, 1H), 4.60-4.55 (m, 1H), 3.50-3.45 (m, 20H), 3.44-3.38 (m, 6H), 2.90-2.82 (m, 2H).

Step 4. To a solution of 2-[[5-bromo-2-[4-[2-[2-[2-[2-[2-[2-(2-hydroxyethoxy) ethoxy]ethoxy]ethoxy]ethoxy]ethoxy]ethylsulfamoyl]anilino]pyrimidin-4-yl]amino]-6-fluoro-benzamide (2.6 g, 3.29 mmol, 1 equiv) and DIEA (965 mg, 7.46 mmol, 1.30 mL, 2.27 equiv) in DCM (30 mL) was added TosCl (780.00 mg, 4.09 mmol, 1.24 equiv) and DMAP (40.0 mg, 329.25 μmol, 0.1 equiv). The mixture was stirred at 20° C. for 60 h. The mixture was washed with H₂O (20 mL), and the DCM layer was evaporated. The residue was purified by prep-HPLC (column: Phenomenex luna C18 (250*70 mm*10 μm); mobile phase: [water(FA)-ACN]; B%: 40%-70%, 23 min) to give the 2-[2-[2-[2-[2-[2-[2-[[4-[[5-bromo-4-(2-carbamoyl-3-fluoro-anilino)pyrimidin-2-yl]amino]phenyl]sulfonylamino]ethoxy]ethoxy]ethoxy]ethoxy]ethoxy]ethoxy]ethyl4-methyl benzenesulfonate (1.3 g, 1.38 mmol, 41.83% yield) as a off-white solid. ¹H NMR (400 MHz, CDCl₃) δ11.49 (s, 1H), 8.46-8.39 (m, 1H), 8.26 (s, 1H), 7.83-7.68 (m, 7H), 7.50-7.42 (m, 1H), 7.40-7.36 (m, 2H), 6.93-6.86 (m, 1H), 6.85-6.75 (m, 1H), 6.22 (s, 1H), 5.85-5.77 (m, 1H), 4.19-4.10 (m, 2H), 3.68-3.61 (m, 13H), 3.57-3.53 (m, 5H), 3.52-3.48 (m, 6H), 3.17-3.09 (m, 2H), 2.44 (s, 3H).

Step 5. To a solution of 2-[2-[2-[2-[2-[2-[2-[[4-[[5-bromo-4-(2-carbamoyl-3-fluoro-anilino)pyrimidin-2-yl]amino]phenyl]sulfonylamino]ethoxy]ethoxy]ethoxy]ethoxy]ethoxy]ethoxy]ethyl 4-methylbenzenesulfonate (1.1 g, 1.17 mmol, 1 equiv) and 3-methoxy-4-nitro-1H-pyrazole (180 mg, 1.26 mmol, 1.08 equiv) in DMF (8 mL) was added Cs₂CO₃ (456.0 mg, 1.40 mmol, 1.2 equiv). The mixture was stirred at 100° C. for 1 h. The mixture was filtered and evaporated. The residue was purified by prep-HPLC (column: Welch Ultimate XB—SiOH 250*70*10 m; mobile phase: [Hexane-EtOH (0.1% NH₃.H₂O)]; B%: 25%-65%, 15 min) to give the 2-[[5-bromo-2-[4-[2-[2-[2-[2-[2-[2-[2-(3-methoxy-4-nitro-pyrazol-1-yl)ethoxy]ethoxy]ethoxy]ethoxy]ethoxy]ethoxy]ethylsulfamoyl]anilino]pyrimidin-4-yl]amino]-6-fluoro-benzamide (580 mg, 634.05 μmol, 54.40% yield) as a yellow gum. ¹H NMR (400 MHz, CDCl₃) δ 11.63 (s, 1H), 8.42-8.37 (m, 1H), 8.26-8.23 (m, 2H), 7.81-7.76 (m, 2H), 7.75-7.71 (m, 2H), 7.52-7.44 (m, 1H), 6.96-6.90 (m, 1H), 6.88-6.81 (m, 1H), 6.22 (s, 1H), 5.85-5.77 (m, 1H), 4.18-4.13 (m, 2H), 4.02 (s, 3H), 3.85-3.80 (m, 2H), 3.66-3.60 (m, 16H), 3.57-3.54 (m, 2H), 3.53-3.47 (m, 4H), 3.17-3.10 (m, 2H).

Step 6. To a solution of 2-[[5-bromo-2-[4-[2-[2-[2-[2-[2-[2-[2-(3-methoxy-4-nitro-pyrazol-1-yl)ethoxy]ethoxy]ethoxy]ethoxy]ethoxy]ethoxy]ethylsulfamoyl]anilino]pyrimidin-4-yl]amino]-6-fluoro-benzamide (480 mg, 524.73 μmol, 1 equiv) in EtOH (15 mL) and H₂O (3 mL) were added Fe (100 mg, 1.79 mmol, 3.41 equiv) and NH₄Cl (150 mg, 2.80 mmol, 5.34 equiv). The mixture was stirred at 80° C. for 4 h. The mixture was filtered. The filtrate was added H₂O (20 mL), and extracted with DCM (2×20 mL). The combined DCM layer was dried over Na₂SO₄, filtered and the filtrate was evaporated. 2-[[2-[4-[2-[[2[2-[2-[2-[2-(4-amino-3-methoxy-pyrazol-1-yl)ethoxy]ethoxy]ethoxy]ethoxy]ethoxy]ethoxy]ethylsulfamoyl]anilino]-5-bromo-pyrimidin-4-yl]amino]-6-fluoro-benzamide (450 mg, crude) was obtained as a yellow gum.

Step 7. To a solution of 2-[[2-[4-[2[2[2-[2-[2-[2-[2-(4-amino-3-methoxy-pyrazol-1-yl)ethoxy]ethoxy]ethoxy]ethoxy]ethoxy]ethoxy]ethylsulfamoyl]anilino]-5-bromo-pyrimidin-4-yl]amino]-6-fluoro-benzamide (450 mg, 508.61 μmol, 1 equiv) and DIEA (148 mg, 1.15 mmol, 0.2 mL, 2.26 equiv) in DMF (4 mL) was added 2,6-dichloro-9-methyl-purine (115 mg, 566.42 μmol, 1.11 equiv). The mixture was stirred at 120° C. for 3 h. The mixture was evaporated. The residue was purified by prep-HPLC (column: Phenomenex luna C18 150*25 mm*10 μm; mobile phase: [water(FA)-ACN]; B%: 38%-68%, 10 min) to give the 2-[[5-bromo-2-[4-[2-[2-[2-[2-[2-[2-[2-[4-[(2-chloro-9-methyl-purin-6-yl)amino]-3-methoxy-pyrazol-1-yl]ethoxy]ethoxy]ethoxy]ethoxy]ethoxy]ethoxy]ethylsulfamoyl]anilino]pyrimidin-4-yl]amino]-6-fluoro-benzamide (330 mg) as a brown solid. ¹H NMR (400 MHz, CDCl₃) δ11.89 (s, 1H), 9.29 (s, 1H), 8.33-8.27 (m, 1H), 8.10-8.05 (m, 1H), 7.82-7.69 (m, 5H), 7.65 (s, 1H), 7.49-7.39 (m, 1H), 7.01-6.88 (m, 2H), 6.26 (s, 1H), 5.81 (s, 1H), 4.20-4.12 (m, 1H), 3.94 (s, 3H), 3.85-3.79 (m, 5H), 3.64-3.54 (m, 18H), 3.52-3.48 (m, 4H), 3.17-3.12 (m, 2H).

Step 8. To a solution of 2-[[5-bromo-2-[4-[2-[2-[2-[2-[2-[2-[2-[4-[(2-chloro-9-methyl-purin-6-yl)amino]-3-methoxy-pyrazol-1-yl]ethoxy]ethoxy]ethoxy]ethoxy]ethoxy]ethoxy]ethylsulfamoyl]anilino]pyrimidin-4-yl]amino]-6-fluoro-benzamide (310 mg, 294.86 μmol, 1 equiv) and tert-butyl N-[(3R,4R)-4-fluoropyrrolidin-3-yl]carbamate (100 mg, 489.62 μmol, 1.66 equiv) in n-BuOH (4 mL) was added DIEA (78 mg, 599.75 μmol, 104.47 μL, 2.03 equiv). The mixture was stirred at 120° C. for 12 h. The mixture was evaporated. The residue was purified by prep-HPLC (column: Waters Xbridge 150*25 mm*5 m; mobile phase: [water(NH4HCO₃)-ACN]; B%: 38%-68%, 9 min) to give the tert-butyl N-[(3R,4R)-1-[6-[[1-[2-[121[2-[2-[12-[2-[[4-[[5-bromo-4-(2-carbamoyl-3-fluoro-anilino)pyrimidin-2-yl]amino]phenyl]sulfonylamino]ethoxy]ethoxy]ethoxy]ethoxy]ethoxy]ethoxy]ethyl]-3-methoxy-pyrazol-4-yl]amino]-9-methyl-purin-2-yl]-4-fluoro-pyrrolidin-3-yl]carbamate (80 mg) as a light yellow solid. ¹H NMR (400 MHz, CDCl₃) δ11.46 (s, 1H), 8.49-8.30 (m, 1H), 8.26-8.02 (m, 2H), 8.00-7.80 (m, 2H), 7.77-7.66 (m, 4H), 7.55-7.32 (m, 2H), 6.99-6.81 (m, 2H), 6.19-6.09 (m, 1H), 5.28-5.03 (m, 1H), 4.40-4.27 (m, 1H), 4.17-4.07 (m, 2H), 3.97-3.77 (m, 9H), 3.74-3.68 (m, 3H), 3.61-3.48 (m, 22H), 3.14-3.07 (m, 2H), 1.47-1.43 (m, 9H).

Step 9. To a solution of tert-butyl N-[(3R,4R)-1-[6-[[1-[2-[2-[2-[2-[2-[2-[2-[[4-[[5-bromo-4-(2-carbamoyl-3-fluoro-anilino)pyrimidin-2-yl]amino]phenyl]sulfonylamino]ethoxy]ethoxy]ethoxy]ethoxy]ethoxy]ethoxy]ethyl]-3-methoxy-pyrazol-4-yl]amino]-9-methyl-purin-2-yl]-4-fluoro-pyrrolidin-3-yl]carbamate (50 mg, 41.01 μmol, 1 equiv) in DCM (3 mL) was added TFA (924.0 mg, 8.10 mmol, 0.6 mL, 197.59 equiv). The mixture was stirred at 20° C. for 1 h. The mixture was evaporated. 2-[[2-[4-[2-[2-[2-[2-[2-[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy]ethoxy]ethoxy]ethoxy]ethoxy]ethylsulfamoyl]anilino]-5-bromo-pyrimidin-4-yl]amino]-6-fluoro-benzamide (51 mg, crude, TFA salt) was obtained as a yellow gum.

Step 10. To a solution of 2-[[2-[4-[2-[2-[2-[2-[2-[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy]ethoxy]ethoxy]ethoxy]ethoxy]ethylsulfamoyl]anilino]-5-bromo-pyrimidin-4-yl]amino]-6-fluoro-benzamide (51 mg, 41.36 μmol, 1 equiv, TFA salt) and NaHCO₃ (21 mg, 249.98 μmol, 9.72 μL, 6.04 equiv) in THF (5 mL) and H₂O (1 mL) was added prop-2-enoyl prop-2-enoate (6.52 mg, 51.70 μmol, 1.25 equiv). The mixture was stirred at 0° C. for 1 h. The mixture was evaporated. The residue was purified with prep-HPLC (column: Waters Xbridge 150*25 mm*5 m; mobile phase: [water(NH₄HCO₃)-ACN]; B%: 29%-59%, 8 min) to give the product. QC-LCMS showed the purity was about 90%. The product was re-purified by prep-HPLC (column: Unisil 3-100 C18 Ultra 150*50 mm*3 m; mobilephase: [water(FA)-ACN]; B%: 30%-60%, 10 min) to give the 2-[[5-bromo-2-[4-[2-[2-[2-[2-[2-[2-[2-[4-[[2-[(3R,4R)-3- fluoro-4-(prop-2-enoylamino)pyrrolidin-1-yl]-9-methyl-purin-6-yl]amino]-3-methoxy-pyrazol-1-yl]ethoxy]ethoxy]ethoxy]ethoxy]ethoxy]ethoxy]ethylsulfamoyl]anilino]pyrimidin-4-yl]amino]-6-fluoro-benzamide (10.64 mg, 8.64 μmol, 20.88% yield, 95.22% purity) as a white solid. ¹H NMR (400 MHz, CDCl₃) δ11.34 (s, 1H), 8.40-8.34 (m, 1H), 8.23 (s, 1H), 8.02 (s, 1H), 7.83 (s, 1H), 7.70-7.60 (m, 4H), 7.47 (s, 1H), 7.43-7.34 (m, 1H), 7.22 (s, 1H), 7.16-7.06 (m, 1H), 7.04-6.94 (m, 1H), 6.89-6.81 (m, 1H), 6.40-6.33 (m, 1H), 6.26-6.17 (m, 1H), 6.15-6.00 (m, 2H), 5.66-5.59 (m, 1H), 5.32-5.14 (m, 1H), 4.72-4.62 (m, 1H), 4.16-4.09 (m, 2H), 3.98-3.82 (m, 7H), 3.79-3.74 (m, 2H), 3.65 (s, 3H), 3.57-3.46 (m, 18H), 3.42-3.34 (m, 4H), 3.16-3.08 (m, 2H). LC-MS: MS (ES′): RT=2.409 min, m/z=1174.3 [M+H⁺]; LCMS Method: 10.

### Example 128—Synthesis of Compound I-144

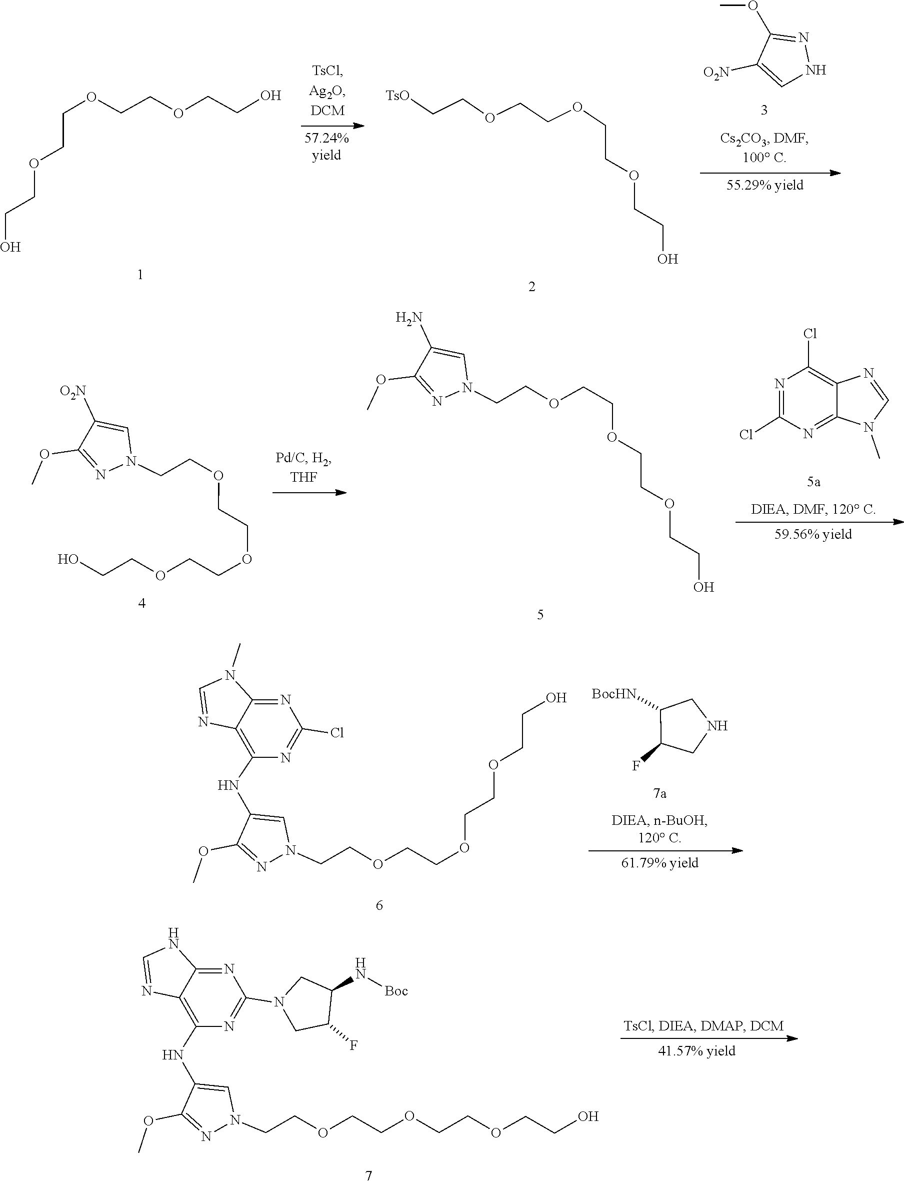

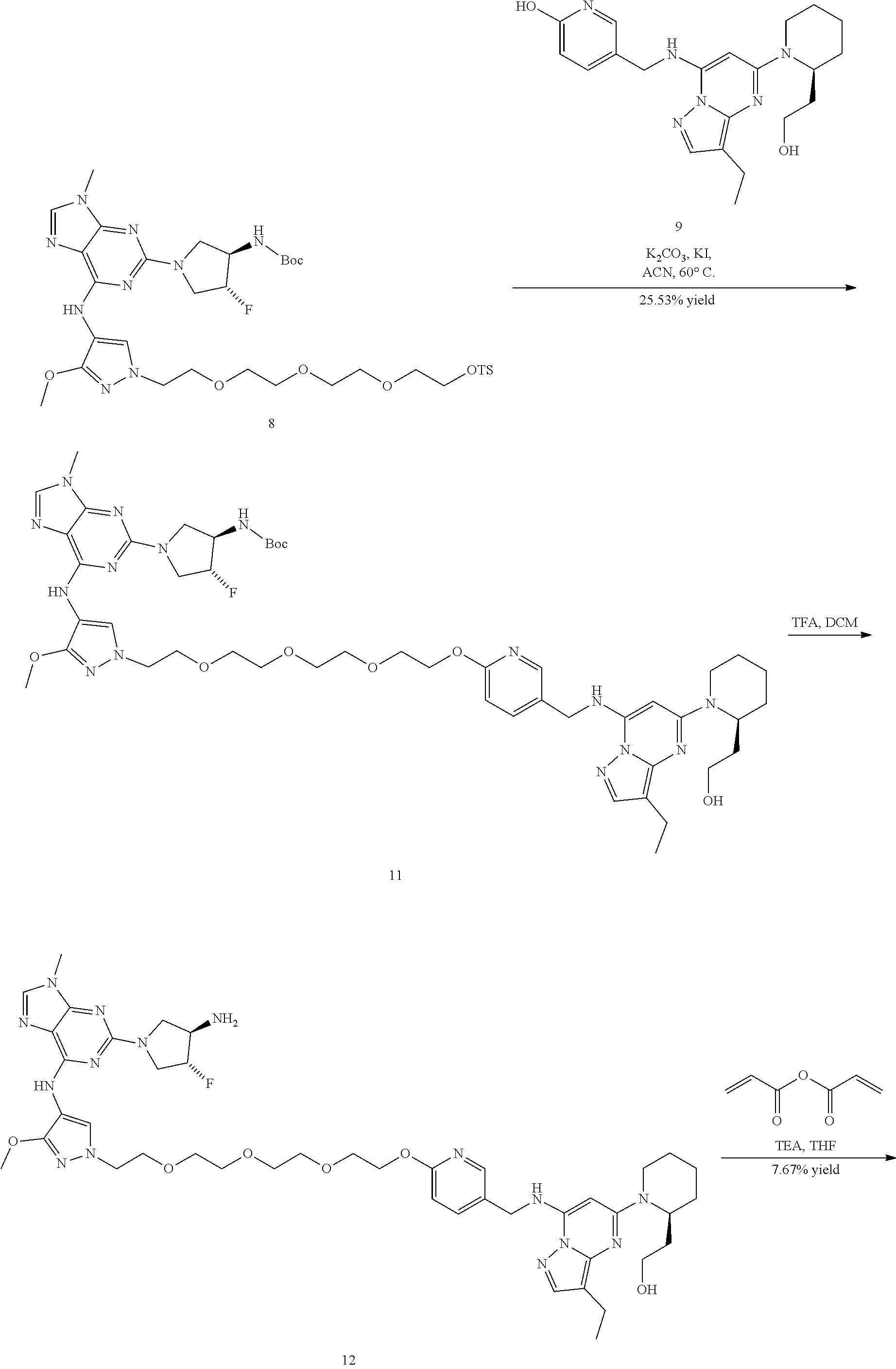

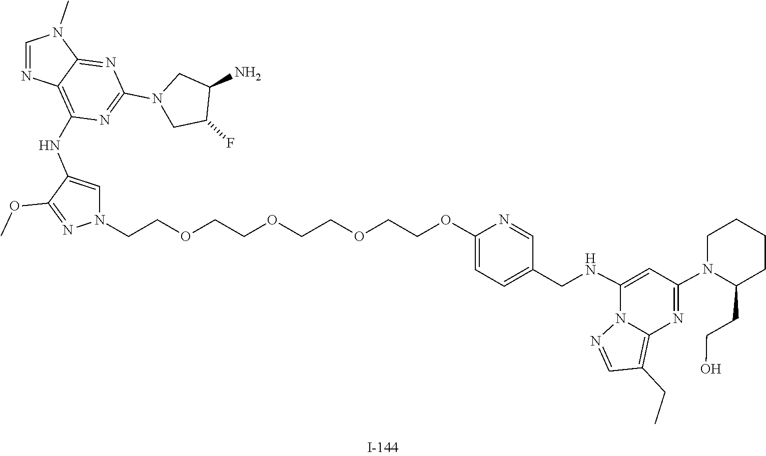 [SMILES: CCc1cnn2c(NCc3ccc(OCCOCCOCCOCCn4cc(Nc5nc(N6C[C@@H](N)[C@H](F)C6)nc6c5ncn6C)c(OC)n4)nc3)cc(N3CCCC[C@H]3CCO)nc12]

Step 1. To a stirred solution of 2-[2-(2-hydroxyethoxy) ethoxy]ethanol (5 g, 33.30 mmol, 4.46 mL, 1 equiv), Ag₂0 (11.57 g, 49.94 mmol, 1.5 equiv) and KI (1.11 g, 6.66 mmol, 0.2 equiv) in DCM (50 mL) was added TosCl (6.98 g, 36.62 mmol, 1.1 equiv) at 0° C. The mixture was stirred at 20° C. for 10 h. TLC (Petroleum ether/Ethyl acetate=1:1) indicated compound 1 was consumed completely and two new spots formed. The reaction mixture was filtered and concentrated under reduced pressure to give a residue. The residue was purified by column chromatography (SiO₂, Petroleum ether/Ethyl acetate=5/1 to 1/1) to give compound 2-[2-(2-hydroxyethoxy) ethoxy]ethyl 4-methylbenzenesulfonate (5.8 g, 19.06 mmol, 57.24% yield) as a colorless oil. ¹H NMR (400 MHz, CDCl₃) δ7.81 (2H, d, J=8.0 Hz), 7.35 (2H, d, J=8.0 Hz), 4.16-4.20 (2H, m), 3.68-3.76 (4H, m), 3.54-3.64 (6H, m), 2.45 (3H, s), 2.28 (1H, s).

Step 2. To a stirred solution of 2-[2-(2-hydroxyethoxy) ethoxy]ethyl 4-methyl benzenesulfonate (1 g, 3.29 mmol, 4.46 mL, 1 equiv), Cs₂CO₃ (1.20 g, 3.68 mmol, 1.12 equiv) in DMF (10 mL) was added 3-methoxy-4-nitro-1H-pyrazole (480.00 mg, 3.35 mmol, 1.02 equiv). The mixture was stirred at 100° C. for 1 h. TLC (Petroleum ether/Ethyl acetate=1:1) indicated compound 2 was consumed completely and one new spot formed. And LCMS showed one peak with desired Ms was detected. The mixture was diluted with EtOAc (30 mL), and washed by water (30 mL), brine (30 mL), the organic layer was dried over Na₂SO₄, concentrated by vacuo. The residue was purified by column chromatography (SiO₂, Petroleum ether/Ethyl acetate=2/1 to 1/1) to give compound 2-[2-[2-(3-methoxy-4-nitro-pyrazol-1-yl)ethoxy]ethoxy]ethanol (0.5 g, 1.82 mmol, 55.29% yield) as a colorless oil.

Step 3. To a solution of 2-[2-[2-(3-methoxy-4-nitro-pyrazol-1-yl) ethoxy]ethoxy]ethanol (0.3 g, 1.09 mmol, 1 equiv) in THF (5 mL) was added Pd/C (10 mg, 10% purity) under N₂. The suspension was degassed under vacuum and purged with H₂ several times. The mixture was stirred under H₂ (2.20 mg, 1.09 mmol, 1 equiv) (15 psi) at 20° C. for 2 h. TLC (Dichloromethane: Methanol=10:1) indicated compound 4 was consumed completely and one new spot formed. The reaction mixture was filtered and the filtrate was concentrated. The residue was purified by prep-TLC (SiO₂, DCM: MeOH=10:1) to give compound 2-[2-[2-(4-amino-3-methoxy-pyrazol-1-yl)ethoxy]ethoxy]ethanol (0.27 g, crude) as a yellow oil.

Step 4. To a stirred solution of 2-[2-[2-(4-amino-3-methoxy-pyrazol-1-yl)ethoxy]ethoxy]ethanol (0.27 g, 1.10 mmol, 1 equiv), DIEA (445.20 mg, 3.44 mmol, 0.6 mL, 3.13 equiv) in DMF (5 mL) was added 2,6-dichloro-9-methyl-purine (0.22 g, 1.08 mmol, 9.84e¹ equiv). The mixture was stirred at 120° C. for 3 h. LCMS showed one peak with desired mass was detected. The mixture was diluted with EtOAc (30 mL), and washed by water (30 mL), brine (30 mL), the organic layer was dried over Na₂SO₄, concentrated by vacuo. The residue was purified by prep-HPLC (FA condition: column: Phenomenex Synergi C18 150*25 mm*10 m; mobile phase:[water(FA)-ACN]; B%: 10%-40%, 10 min) to give compound 2-[2-[2-[4-[(2-chloro-9-methyl-purin-6-yl)amino]-3-methoxy-pyrazol-1-yl]ethoxy]ethoxy]ethanol (0.27 g, 655.59 μmol, 59.56% yield) as a yellow solid. ¹H NMR (400 MHz, CDCl₃) δ7.81 8.03 (1H, s), 7.67 (1H, s), 7.52 (1H, s), 4.10 (2H, t, J=5.2 Hz), 3.90 (3H, s), 3.78 (2H, t, J=5.2 Hz), 3.75 (3H, s), 3.62-3.66 (2H, m), 3.57-3.61 (2H, m), 3.51-3.56 (4H, m).

Step 5. To a stirred solution of 2-[2-[2-[4-[(2-chloro-9-methyl-purin-6-yl)amino]-3-methoxy-pyrazol-1-yl]ethoxy]ethoxy]ethanol (0.23 g, 558.47 μmol, 1 equiv), DIEA (225.86 mg, 1.75 mmol, 304.39 μL, 3.13 equiv) in n-BuOH (5 mL) was added tert-butyl N-[(3R,4R)-4-fluoropyrrolidin-3-yl]carbamate (0.23 g, 1.13 mmol, 2.02 equiv). The mixture was stirred at 120° C. for 10 h. LCMS showed one peak with desired mass was detected. The reaction mixture was concentrated under reduced pressure to give a residue. The residue was purified by prep-HPLC (FA condition: column: Phenomenex Synergi C18 150*25 mm*10 m; mobile phase: [water(FA)-ACN]; B%: 24%-54%, 10 min) to give compound tert-butyl N-[(3R,4R)-4-fluoro-1-[6-[[1-[2-[2-(2-hydroxyethoxy)ethoxy]ethyl]-3-methoxy-pyrazol-4-yl]amino]-9-methyl-purin-2-yl]pyrrolidin-3-yl]carbamate (0.2 g, 345.05 μmol, 61.79% yield) as a yellow solid.

Step 6. To a solution of tert-butyl N-[(3R,4R)-4-fluoro-1-[6-[[1-[2-[2-(2-hydroxyethoxy)ethoxy]ethyl]-3-methoxy-pyrazol-4-yl]amino]-9-methyl-purin-2-yl]pyrrolidin-3-yl]carbamate (0.19 g, 327.80 μmol, 1 equiv), DIEA (103.88 mg, 803.76 μmol, 140.00 μL, 2.45 equiv) and DMAP (10 mg, 81.85 μmol, 0.25 equiv) in DCM (5 mL) was added TosCl (0.12 g, 629.44 μmol, 1.92 equiv) at 20° C. and stirred at 20° C. for 10 hours. LC-MS showed compound 7 was remained and one peak with desired mass was detected. The reaction mixture was concentrated under reduced pressure to remove solvent. The residue was purified by prep-HPLC (neutral condition: column: Waters Xbridge 150*25 mm*5 m; mobile phase:[water(NH₄HCO₃)-ACN]; B%: 47%-77%, 10 min) to give compound 2-[2-[2-[4-[[2-[(3R,4R)-3-(tert-butoxycarbonylamino)-4-fluoro-pyrrolidin-1-yl]-9-methyl-purin-6-yl]amino]-3-methoxy-pyrazol-1-yl]ethoxy]ethoxy]ethyl 4-methylbenzenesulfonate (0.1 g, 136.28 μmol, 41.57% yield) as a yellow solid. ¹H NMR (400 MHz, CDCl₃) δ7.89 (1H, d, J 1.2 Hz), 7.56-7.75 (4H, m), 7.35-7.52 (2H, m), 4.81-5.45 (2H, m), 4.31 (1H, d, J=3.2 Hz), 4.06 (2H, t, J=5.2 Hz), 3.70-3.99 (11H, m), 3.65 (3H, s), 3.51-3.56 (2H, m), 3.48 (4H, s), 2.33 (3H, s), 1.38 (9H, s).

Step 7. To a solution of 2-[2-[2-[4-[[2-[(3R,4R)-3-(tert-butoxycarbonylamino)-4-fluoro-pyrrolidin-1-yl]-9-methyl-purin-6-yl]amino]-3-methoxy-pyrazol-1-yl]ethoxy]ethoxy]ethyl 4-methylbenzenesulfonate (90 mg, 122.65 μmol, 1 equiv), K₂CO₃ (50.85 mg, 367.94 μmol, 5.82 μL, 3 equiv) and KI (30.54 mg, 183.97 μmol, 1.5 equiv) in CH₃CN (3 mL) was added 5-[[[3-ethyl-5-[(2S)-2-(2-hydroxyethyl)-1-piperidyl]pyrazolo[1,5-a]pyrimidin-7-yl]amino]methyl]pyridin-2-ol (63.00 mg, 158.90 μmol, 1.30 equiv) at 20° C. and stirred at 80° C. for 10 h. LCMS showed one peak with desired mass was detected. The reaction mixture was concentrated under reduced pressure to remove solvent. The residue was purified by prep-HPLC (neutral condition:column: Unisil 3-100 C18 Ultra 150*50 mm*3 m; mobile phase: [water(FA)-ACN]; B%: 25%-55%, 10 min) to give compound tert-butyl N-[(3R,4R)-1-[6-[[1-[2-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethyl]-3-methoxy-pyrazol-4-yl]amino]-9-methyl-purin-2-yl]-4-fluoro-pyrrolidin-3-yl]carbamate (30 mg, 31.31 μmol, 25.53% yield) as a white solid. ¹H NMR (400 MHz, CDCl₃) δ7.93-8.01 (2H, m), 7.56 (1H, s), 7.38-7.47 (2H, m), 6.61 (1H, d, J=8.4 Hz), 6.31 (1H, s), 5.23 (1H, s), 4.92-5.20 (3H, m), 4.23-4.36 (5H, m), 4.07 (2H, t, J=5.2 Hz), 3.80-3.91 (6H, m), 3.66-3.78 (6H, m), 3.48-3.62 (9H, m), 3.24-3.32 (1H, m), 2.91-3.02 (1H, m), 2.53 (2H, m), 2.00 (2H, m), 1.73 (1H, m), 1.50-1.66 (6H, m), 1.36 (9H, s), 1.18 (3H, t, J=7.6 Hz).

Step 8. To a solution of tert-butyl N-[(3R,4R)-1-[6-[[1-[2-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethyl]-3-methoxy-pyrazol-4-yl]amino]-9-methyl-purin-2-yl]-4-fluoro-pyrrolidin-3-yl]carbamate (25 mg, 26.09 μmol, 1 equiv) in DCM (2 mL) was added TFA (3.85 g, 33.77 mmol, 2.50 mL, 1294.00 equiv) at 20° C. and stirred at 20° C. for 0.5 h. LC-MS showed one peak with desired mass was detected. The reaction mixture was concentrated under reduced pressure to remove solvent. The colorless oil crude product 2-[(2S)-1-[7-[[6-[2-[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy]ethoxy]-3-pyridyl]methylamino]-3-ethyl-pyrazolo [1,5-a]pyrimidin-5-yl]-2-piperidyl]ethanol (30 mg, crude, TFA) was used into the next step without further purification.

Step 9. To a solution of 2-[(2S)-1-[7-[[6-[2-[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy]ethoxy]-3-pyridyl]methylamino]-3-ethyl-pyrazolo[1,5-a]pyrimidin-5-yl]-2-piperidyl]ethanol (25 mg, 25.72 μmol, 1 equiv, TFA) and TEA (6.51 mg, 64.30 μmol, 8.95 μL, 2.5 equiv) in THF (5 mL) was added prop-2-enoyl prop-2-enoate (3.89 mg, 30.86 μmol, 1.2 equiv) at 20° C. and stirred at 20° C. for 0.5 h. LCMS showed one peak with desired mass was detected. The reaction mixture was concentrated under reduced pressure to remove solvent. The residue was purified by prep-HPLC (FA condition: column: Phenomenex Synergi C18 150*25 mm*10 m; mobile phase:[water(FA)-ACN]; B%: 24%-40%, 8 min) to give compound N-[(3R,4R)-1-[6-[[1-[2-[2-[2-[[5-1[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethyl]-3-methoxy-pyrazol-4-yl]amino]-9-methyl-purin-2-yl]-4-fluoro-pyrrolidin-3-yl]prop-2-enamide (1.89 mg, 1.97 μmol, 7.67% yield, 95.15% purity) as a white solid. ¹H NMR (400 MHz, DMSO-d₆) δ7.96-8.14 (2H, m), 7.65 (1H, s), 7.45-7.54 (2H, m), 7.24 (1H, s), 6.82 (1H, d, J=5.2 Hz), 6.63 (1H, d, J=8.4 Hz), 6.34-6.42 (2H, m), 6.09-6.20 (1H, m), 5.65 (1H, d, J=11.2 Hz), 5.20-5.38 (2H, m), 5.06-5.14 (1H, m), 4.66-4.75 (1H, m), 4.36 (2H, d, J=5.2 Hz), 4.30 (2H, t, J=4.4 Hz), 4.16 (2H, t, J=5.2 Hz), 3.97 (3H, s), 3.93 (3H, s), 3.82 (3H, t, J=4.8 Hz), 3.61-3.76 (11H, m), 3.33-3.41 (1H, m), 2.98-3.08 (1H, m), 2.60-2.67 (2H, m), 2.05-2.15 (2H, m), 1.51-1.66 (7H, m), 1.27 (4H, t, J=7.6 Hz). LC-MS: MS (ES′): RT=1.806 min, m/z=912.3 [M+H⁺]; LCMS Method: 25.

### Example 129—Synthesis of Compound I-145

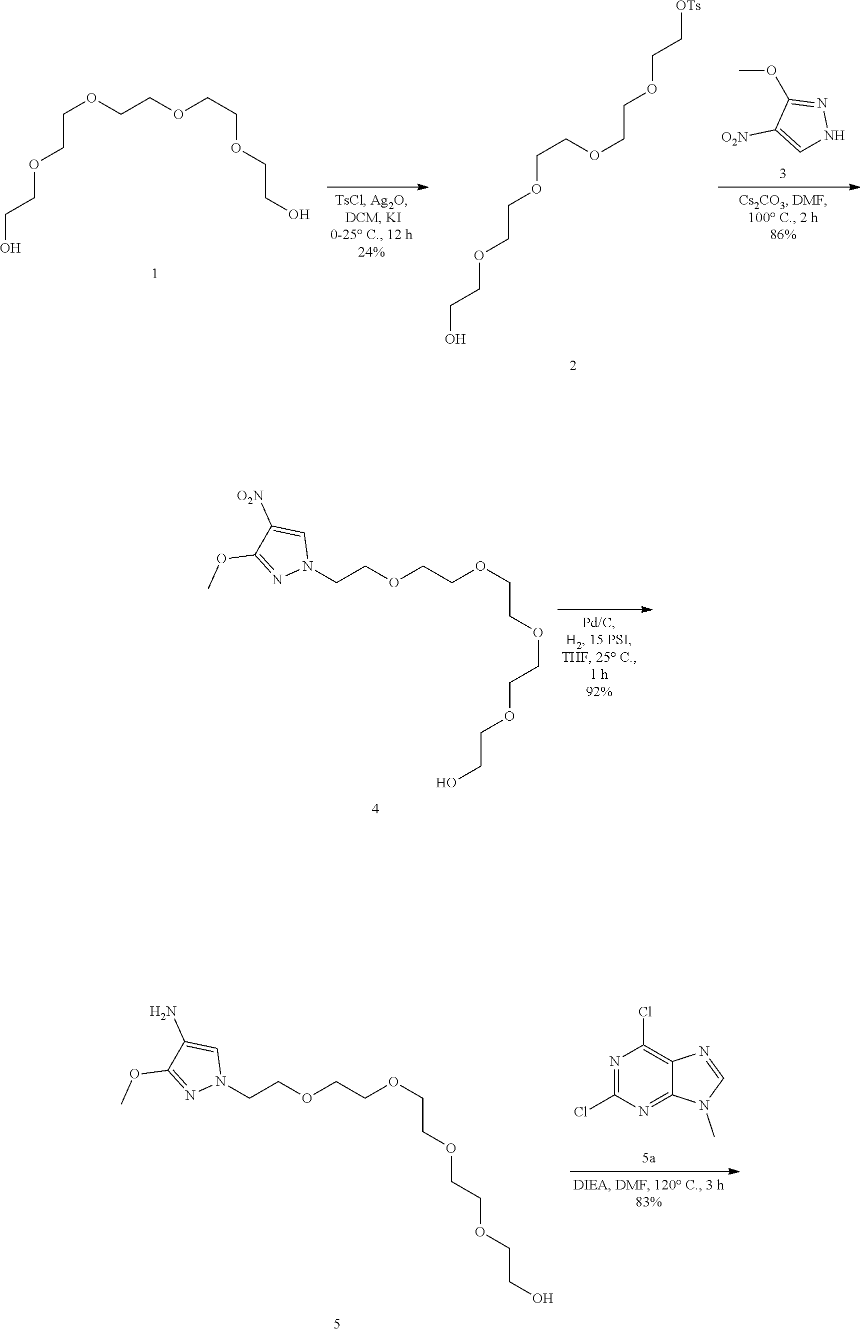

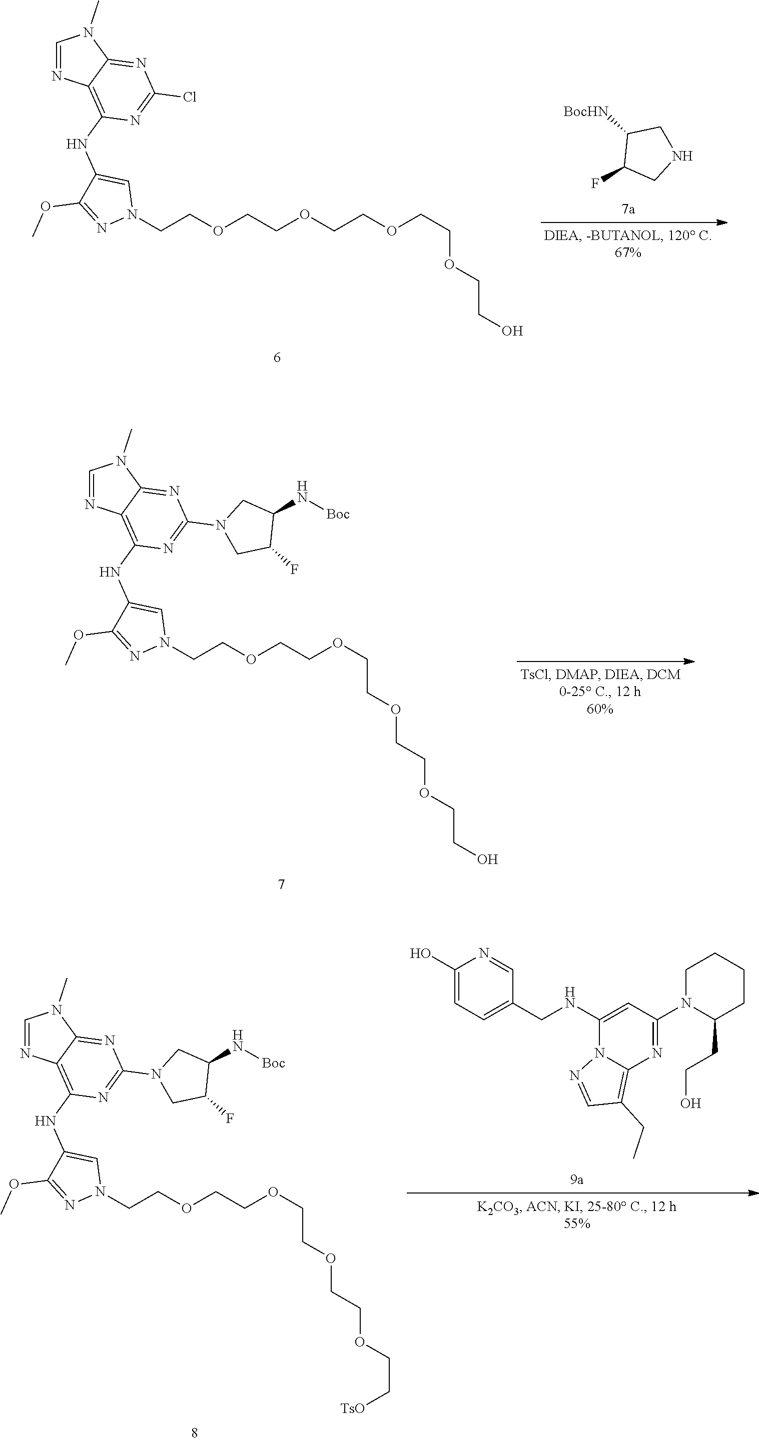 [SMILES: COc1nn(CCOCCOCCOCCOCCO)cc1Nc1nc(Cl)nc2c1ncn2C ; CC(C)(C)OC(=O)N[C@@H]1CNC[C@H]1F ; COc1nn(CCOCCOCCOCCOCCO)cc1Nc1nc(N2C[C@@H](F)[C@H](NC(=O)OC(C)(C)C)C2)nc2c1ncn2C ; COc1nn(CCOCCOCCOCCOCCOS(=O)(=O)c2ccc(C)cc2)cc1Nc1nc(N2C[C@@H](F)[C@H](NC(=O)OC(C)(C)C)C2)nc2c1ncn2C ; CCc1cnn2c(NCc3ccc(O)nc3)cc(N3CCCC[C@H]3CCO)nc12]

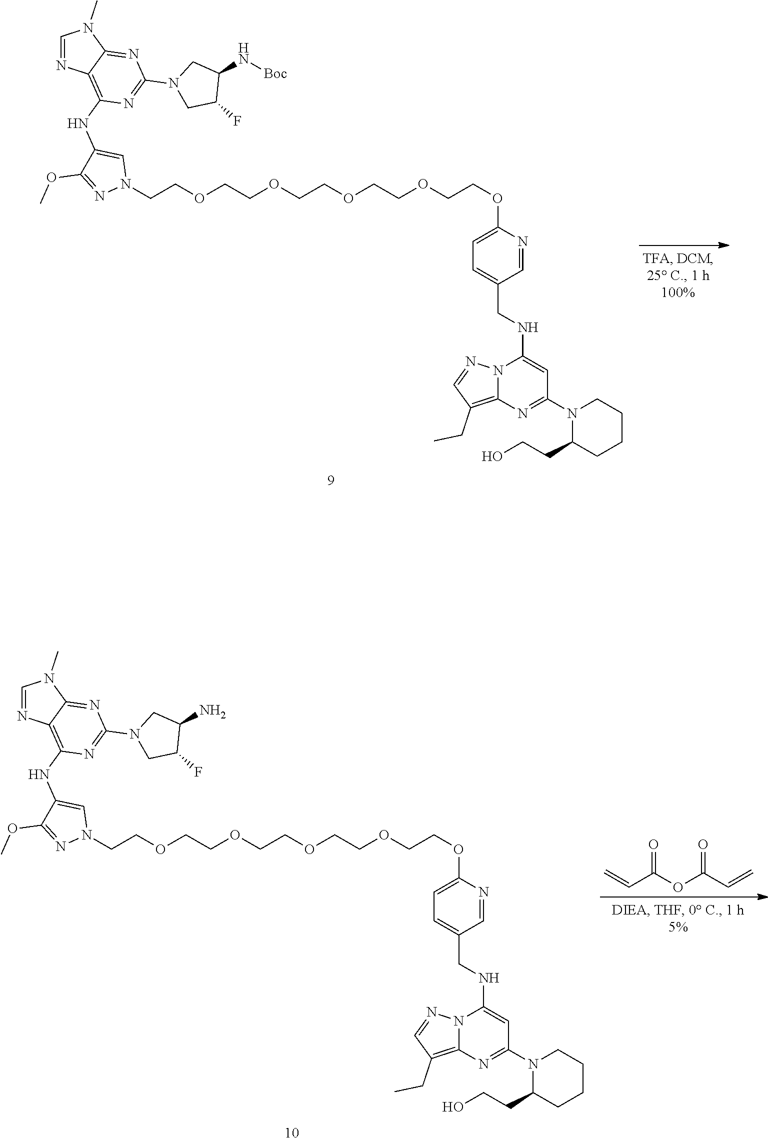

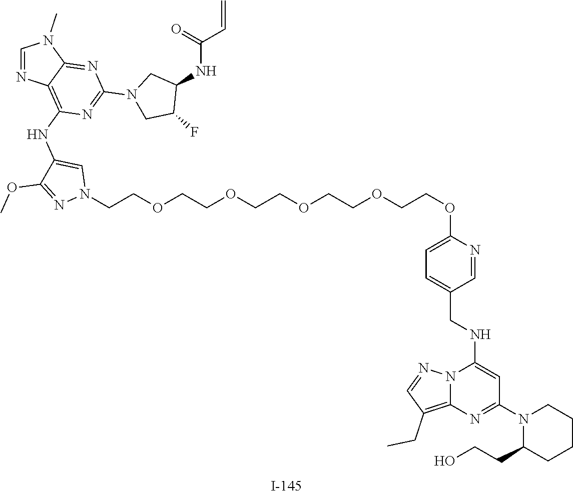 [SMILES: C=CC(=O)N[C@@H]1CN(c2nc(Nc3cn(CCOCCOCCOCCOCCOc4ccc(CNc5cc(N6CCCC[C@H]6CCO)nc6c(CC)cnn56)cn4)nc3OC)c3ncn(C)c3n2)C[C@H]1F]

Step 1. To a solution of 2-[2-[2-[2-(2-hydroxyethoxy)ethoxy]ethoxy]ethoxy]ethanol (5 g, 20.98 mmol, 1 equiv) in DCM (70 mL) were added Ag₂O (7.30 g, 31.50 mmol, 1.5 equiv), KI (3.80 g, 22.89 mmol, 1.09 equiv) and TosCl (4.00 g, 20.98 mmol, 1 equiv) at 0° C. The reaction mixture was stirred at 25° C. for 12 h. TLC (Petroleum ether: Ethyl acetate=1/1) showed that the starting material was consumed completely. The solution was filtered and the filtrate was washed with H₂O (30 ml*2). The combined organic layers were dried over Na₂SO₄, filtered and concentrated. The crude product was purified by column chromatography (Petroleum ether: Ethyl acetate=10/1-1/1) to give 2-[2-[2-[2-(2-hydroxyethoxy)ethoxy]ethoxy]ethoxy]ethyl 4-methyl benzenesulfonate (2 g, 5.10 mmol, 24.29% yield) as a yellow oil. ¹H NMR (400 MHz, CDCl₃) 7.72 (d, J=8.4 Hz, 2H), 7.34-7.24 (m, 2H), 4.11-4.06 (m, 2H), 3.67-3.52 (m, 18H), 2.37 (s, 3H).

Step 2. To a solution of 2-[2-[2-[2-(2-hydroxyethoxy)ethoxy]ethoxy]ethoxy]ethyl 4-methylbenzenesulfonate (1.5 g, 3.82 mmol, 1 equiv) in DMF (20 mL) were added 3-methoxy-4-nitro-1H-pyrazole (500 mg, 3.49 mmol, 9.14e⁻¹ equiv) and Cs₂CO₃ (1.6 g, 4.91 mmol, 1.28 equiv). The reaction mixture was stirred at 100° C. for 2 h. LCMS showed that the starting material was consumed completely and about 93% of the desired product's MS was detected. HPLC showed the purity of the desired product was about 76%. To the solution was added H₂O (30 ml) and extracted with EA (30 ml×5). The combined organic layers were washed with brine (10 ml×2), dried over Na₂SO₄, filtered and concentrated. The crude product was purified by prep-HPLC (column: Phenomenex Luna C8 250*50 mm*10 m; mobile phase: [water (FA)-ACN]; B%: 5%-35%, 20 min) to 2-[2-[2-[2-[2-(3-methoxy-4-nitro-pyrazol-1-yl)ethoxy]ethoxy]ethoxy]ethoxy]ethanol (1.2 g, 3.30 mmol, 86.41% yield) as a colorless oil. ¹H NMR (400 MHz, CDCl₃) 8.23 (s, 1H), 4.17-4.12 (m, 2H), 4.02 (s, 3H), 3.84-3.80 (m, 2H), 3.73-3.68 (m, 2H), 3.66-3.58 (m, 14H), 2.93 (s, 1H).

Step 3. To a solution of Pd/C (100 mg, 10% purity) in THF (20 mL) was added 2-[2-[2-[2-[2-(3-methoxy-4-nitro-pyrazol-1-yl)ethoxy]ethoxy]ethoxy]ethoxy]ethanol (1 g, 2.75 mmol, 1 equiv) under N2 atmosphere. The suspension was degassed under vacuum and purged with H₂ several times. The mixture was stirred under H₂ (15 psi) at 25° C. for 1 h. LCMS showed that the starting material was consumed completely and about 69% of the desired product's MS was detected. The solution was filtered and the filtrate was concentrated to 2-[2-[2-[2-[2-(4-amino-3-methoxy-pyrazol-1-yl) ethoxy]ethoxy]ethoxy]ethoxy]ethanol (850 mg, 2.55 mmol, 92.64% yield) as a colorless oil.

Step 4. To a solution of 2-[2-[2-[2-[2-(4-amino-3-methoxy-pyrazol-1-yl)ethoxy]ethoxy]ethoxy]ethoxy]ethanol (800 mg, 2.40 mmol, 1 equiv) in DMF (10 mL) were added 2,6-dichloro-9-methyl-purine (500 mg, 2.46 mmol, 1.03 equiv) and DIEA (593.60 mg, 4.59 mmol, 800.00 μL, 1.91 equiv). The reaction mixture was stirred at 120° C. for 3 hours. LCMS showed that the starting material was consumed completely and about 83% of the desired product's MS was detected. HPLC showed that the purity of the desired product was about 63%. To the solution was added H₂O (20 ml) and extracted with EA (20 ml*4). The combined organic layers were washed with brine (10 ml×2), dried over Na₂SO₄, filtered and concentrated. The crude product was purified by prep-HPLC (column: Waters Xbridge BEH C18 250*50 mm*10 m; mobile phase: [water (ammonia hydroxide v/v)-ACN]; B%: 15%-45%, 20 min) to give 2-[2[2-[2-2-[4-[(2-chloro-9-methyl-purin-6-yl)amino]-3-methoxy-pyrazol-1-yl]ethoxy]ethoxy]ethoxy]ethoxy]ethanol (1 g, 2.00 mmol, 83.35% yield) as a brown solid. ¹H NMR (400 MHz, CDCl₃) δ8.12 (s, 1H), 7.87 (br s, 1H), 7.77 (s, 1H), 4.18 (t, J=5.2 Hz, 2H), 3.98 (s, 3H), 3.88-3.84 (m, 2H), 3.83 (s, 3H), 3.78-3.74 (m, 2H), 3.65-3.60 (m, 10H), 3.49-3.55 (m, 4H).

Step 5. To a solution of 2-[2-[2-[2-[2-[4-[(2-chloro-9-methyl-purin-6-yl)amino]-3-methoxy-pyrazol-1-yl]ethoxy]ethoxy]ethoxy]ethoxy]ethanol (500 mg, 1.00 mmol, 1 equiv) in 1-butanol (10 mL) were added tert-butyl N-[(3R,4R)-4-fluoropyrrolidin-3-yl]carbamate (410.00 mg, 2.01 mmol, 2.01 equiv), DIEA (296.80 mg, 2.30 mmol, 400.00 μL, 2.30 equiv). The reaction mixture was stirred at 120° C. for 12 h. LCMS showed that about 37% of the starting material still remained and about 52% of the desired product's MS was detected. The solution was concentrated. The crude product was purified by prep-HPLC (column: Phenomenex luna C18 150*40 mm*15 m; mobile phase: [water (FA)-ACN]; B%: 20%-50%, 10 min) to give tert-butyl N-[(3R,4R)-4-fluoro-1-[6-[[1-1[2-[2-[2-1[2-(2-hydroxyethoxy)ethoxy]ethoxy]ethoxy]ethyl]-3-methoxy-pyrazol-4-yl]amino]-9-methyl-purin-2-yl]pyrrolidin-3-yl]carbamate (450 mg, 673.93 μmol, 67.39% yield) as a yellow oil. ¹H NMR (400 MHz, CDCl₃) δ8.04 (s, 1H), 7.80-7.68 (m, 1H), 7.67-7.52 (m, 1H), 5.49 (br s, 1H), 5.33-5.10 (m, 1H), 4.44-4.33 (m, 1H), 4.17 (t, J=5.0 Hz, 2H), 4.05-3.78 (m, 1OH), 3.75-3.68 (m, 5H), 3.62 (s, 3H), 3.59-3.47 (m, 1OH).

Step 6. To a solution of tert-butyl N-[(3R,4R)-4-fluoro-1-[6-[[1-[2-[2-[2-[2-(2-hydroxyethoxy)ethoxy]ethoxy]ethoxy]ethyl]-3-methoxy-pyrazol-4-yl]amino]-9-methyl-purin-2-yl]pyrrolidin-3-yl]carbamate (400 mg, 599.05 μmol, 1 equiv) in DCM (20 mL) were added DMAP (8.00 mg, 65.48 μmol, 1.09e⁻¹ equiv), TosCl (176.00 mg, 923.17 μmol, 1.54 equiv) and DIEA (148.40 mg, 1.15 mmol, 0.2 mL, 1.92 equiv) at 0° C. The reaction mixture was stirred at 25° C. for 12 h. LCMS showed that the starting material was consumed completely and about 77% of the desired product's MS was detected. The solution was concentrated. The crude product was purified by prep-HPLC (column: Phenomenex luna C18 150*40 mm*15 m; mobile phase: [water (FA)-ACN]; B%: 38%-68%, 10 min) to give 2-[2-[2-[2-[2-[4-[[2-[(3R,4R)-3-(tert-butoxycarbonylamino)-4-fluoro-pyrrolidin-1-yl]-9-methyl-purin-6-yl]amino]-3-methoxy-pyrazol-1-yl]ethoxy]ethoxy]ethoxy]ethoxy]ethyl 4-methylbenzenesulfonate (300 mg, 365.00 μmol, 60.93% yield) as a brown gum. ¹H NMR (400 MHz, CDCl₃) δ7.94 (s, 1H), 7.70 (d, J=8.2 Hz, 2H), 7.49 (s, 1H), 7.35 (br d, J=6.0 Hz, 1H), 7.25 (d, J=8.1 Hz, 2H), 5.21-4.95 (m, 2H), 4.29 (br s, 1H), 4.05 (dt, J=16.3, 5.0 Hz, 4H), 3.89 (s, 3H), 3.81 (br s, 2H), 3.74 (br t, J=5.3 Hz, 3H), 3.61 (s, 3H), 3.55-3.47 (m, 8H), 3.45-3.40 (m, 4H), 2.36 (s, 3H), 1.37 (br s, 9H).

Step 7. To a solution of 2-[2-[2-[2-[2-[4-[[2-[(3R,4R)-3-(tert-butoxycarbonylamino)-4-fluoro-pyrrolidin-1-yl]-9-methyl-purin-6-yl]amino]-3-methoxy-pyrazol-1-yl]ethoxy]ethoxy]ethoxy]ethoxy]ethyl 4-methylbenzenesulfonate (85 mg, 103.42 μmol, 1 equiv) in CH₃CN (10 mL) were added 5-[[[3-ethyl-5-[(2S)-2-(2-hydroxyethyl)-1-piperidyl]pyrazolo[1,5-a]pyrimidin-7-yl]amino]methyl]pyridin-2-ol (61 mg, 153.85 μmol, 1.49 equiv) and K₂CO₃ (43 mg, 311.13 μmol, 3.01 equiv), KI (309.01 mg, 1.86 mmol, 18 equiv) at 25° C. The reaction mixture was stirred at 80° C. for 12 h. LCMS showed that about 14% of the starting material still remained and about 54% of the desired product's MS was detected. The solution was filtered and the filtrate was concentrated. The crude product was purified by prep-HPLC (column: Unisil 3-100 C18 Ultra 150*50 mm*3 μm; mobile phase: [water(FA)-ACN]; B%: 20%-50%, 10 min) to give tert-butyl N-[(3R,4R)-1-[6-[[1-[2-[2-[2-[2-[2-[[5-[[[3-ethyl-5-[(2S)-2-(2-hydroxyethyl)-1-piperidyl]pyrazolo1[1,5-a]pyrimidin-7-yl]amino]methyl]-2-pyridyl]oxy]ethoxy]ethoxy]ethoxy]ethoxy]ethyl]-3-methoxy-pyrazol-4-yl]amino]-9-methyl-purin-2-yl]-4-fluoro-pyrrolidin-3-yl]carbamate (60 mg, 57.35 μmol, 55.46% yield) as a brown solid. ¹H NMR (400 MHz, CDCl₃) δ8.13 (d, J=2.4 Hz, 1H), 8.04 (br s, 1H), 7.65 (s, 1H), 7.59 (br dd, J=8.4, 2.4 Hz, 2H), 6.78 (d, J=8.4 Hz, 1H), 5.36-5.24 (m, 1H), 5.18-5.00 (m, 2H), 4.37-4.46 (m, 4H), 4.17 (t, J=5.2 Hz, 2H), 4.01-3.88 (m, 6H), 3.82 (dt, J=9.2, 4.8 Hz, 6H), 3.72 (s, 3H), 3.67-3.53 (m, 14H), 3.44-3.32 (m, 1H), 3.13-3.02 (m, 1H), 2.71-2.55 (m, 2H), 2.18-1.98 (m, 2H), 1.76-1.65 (m, 6H), 1.46 (br s, 9H), 1.26 (br t, J=7.6 Hz, 3H).

Step 8. A mixture of tert-butyl N-[(3R,4R)-1-[6-[[1-[2-[2-[2-[2-[2-[[5-[[[3-ethyl-5-[(2S)-2-(2-hydroxyethyl)-1-piperidyl]pyrazolo[ 1,5-a]pyrimidin-7-yl]amino]methyl]-2-pyridyl]oxy]ethoxy]ethoxy]ethoxy]ethoxy]ethyl]-3-methoxy-pyrazol-4-yl]amino]-9-methyl-purin-2-yl]-4-fluoro-pyrrolidin-3-yl]carbamate (40 mg, 38.23 μmol, 1 equiv) and TFA (616.00 mg, 5.40 mmol, 400.00 μL, 141.30 equiv) in DCM (6 mL) was stirred at 25° C. for 1 h. LCMS showed that the starting material was consumed completely and about 86% of the desired product's MS was detected. The solution was concentrated to give2-[(2S)-1-[7-[[6-[2-[2-[2-[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy]ethoxy]ethoxy]ethoxy]-3-pyridyl]methylamino]-3-ethyl-pyrazolo[1,5-a]pyrimidin-5-yl]-2-piperidyl]ethanol (40 mg, crude, TFA) as a brown oil.

Step 9. To a solution of 2-[(2S)-1-[7-[[6-[2-[2-[2-[2-[2-[4-[[2-[(3R,4R)-3-amino-4-fluoro-pyrrolidin-1-yl]-9-methyl-purin-6-yl]amino]-3-methoxy-pyrazol-1-yl]ethoxy]ethoxy]ethoxy]ethoxy]ethoxy]-3-pyridyl]methylamino]-3-ethyl-pyrazolo[1,5-a]pyrimidin-5-yl]-2-piperidyl]ethanol (40 mg, 37.73 μmol, 1 equiv, TFA) in THF (6 mL) were added prop-2-enoyl prop-2-enoate (8.00 mg, 63.44 μmol, 1.68 equiv), DIEA (105.95 mg, 819.78 μmol, 142.79 μL, 21.73 equiv) at 0° C. The reaction mixture was stirred at 0° C. for 1 h. LCMS showed that the starting material was consumed completely and about 86% of the desired product's MS was detected. The solution was concentrated. The crude product was purified by prep-HPLC (column: Waters Xbridge 150*25 mm*5 m; mobile phase: [water (NH₄HCO₃)-ACN]; B%: 42%-62%, 8 min) to give product N-[(3R,4R)-1-[6-[[1-[2-[2-[2-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethyl]-3-methoxy-pyrazol-4-yl]amino]-9-methyl-purin-2-yl]-4-fluoro-pyrrolidin-3-yl]prop-2-enamide (2.10 mg, 1.95 μmol, 5.18% yield, 93% purity) as a yellow solid. ¹H NMR (400 MHz, CDCl₃) δ8.13 (s, 1H), 8.12 (d, J=2.3 Hz, 1H), 7.65 (s, 1H), 7.58 (dd, J=8.6, 2.4 Hz, 1H), 7.48 (s, 1H), 6.95 (br d, J=6.1 Hz, 1H), 6.76 (d, J=8.6 Hz, 1H), 6.40-6.30 (m, 2H), 6.24-6.16 (m, 1H), 5.62 (dd, J=10.1, 1.7 Hz, 1H), 5.15-5.03 (m, 1H), 4.76-4.61 (m, 1H), 4.43-4.39 (m, 4H), 4.18 (t, J=4.9 Hz, 2H), 4.05 (br s, 1H), 3.98 (s, 3H), 3.94-3.88 (m, 2H), 3.77 (dt, J=12.7, 4.8 Hz, 5H), 3.68 (s, 3H), 3.59-3.56 (m, 6H), 3.54-3.51 (m, 2H), 3.45 (br d, J=5.5 Hz, 2H), 3.39 (d, J=7.1 Hz, 3H), 3.37-3.28 (m, 1H), 3.12-2.99 (m, 1H), 2.86 (s, 3H), 2.61 (qd, J=7.5, 2.9 Hz, 2H), 2.39 (t, J=8.1 Hz, 2H), 2.16-2.03 (m, 3H), 2.03-2.00 (m, 3H), 1.28-1.25 (m, 3H). LC-MS: MS (ES⁺): RT=2.298 min, m/z=1000.4 [M+H⁺]; LCMS Method: 10.

### Example 130—Synthesis of Compound I-146

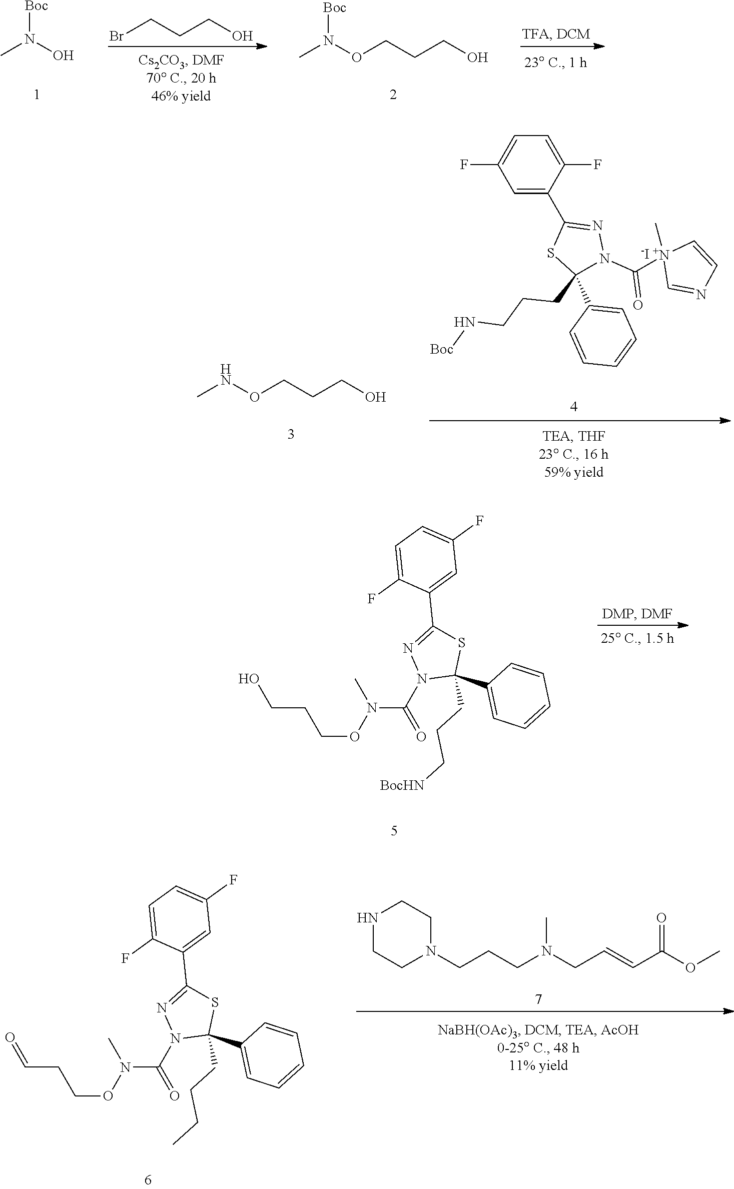

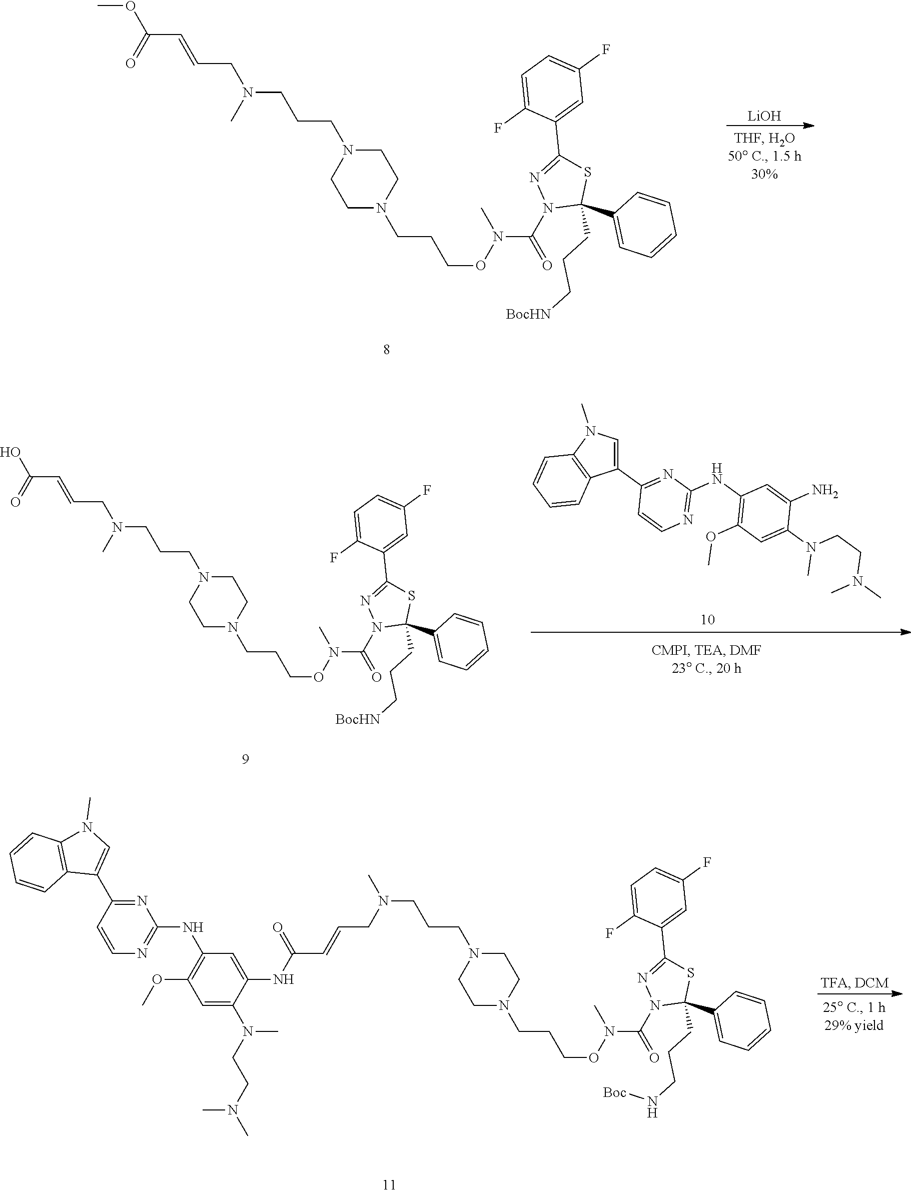 [SMILES: COC(=O)/C=C/CN(C)CCCN1CCN(CCCON(C)C(=O)N2N=C(c3cc(F)ccc3F)S[C@@]2(CCCNC(=O)OC(C)(C)C)c2ccccc2)CC1 ; "LiOH" ; CN(C/C=C/C(=O)O)CCCN1CCN(CCCON(C)C(=O)N2N=C(c3cc(F)ccc3F)S[C@@]2(CCCNC(=O)OC(C)(C)C)c2ccccc2)CC1 ; COc1cc(N(C)CCN(C)C)c(N)cc1Nc1nccc(-c2cn(C)c3ccccc23)n1 ; COc1cc(N(C)CCN(C)C)c(NC(=O)/C=C/CN(C)CCCN2CCN(CCCON(C)C(=O)N3N=C(c4cc(F)ccc4F)S[C@@]3(CCCNC(=O)OC(C)(C)C)c3ccccc3)CC2)cc1Nc1nccc(-c2cn(C)c3ccccc23)n1]

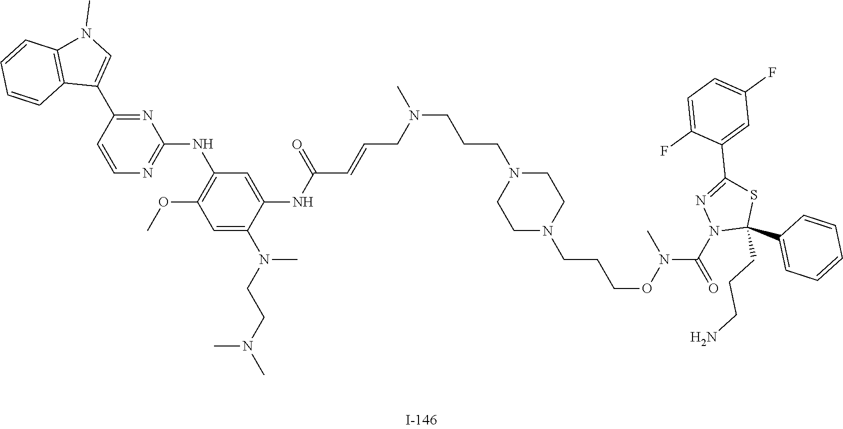 [SMILES: COc1cc(N(C)CCN(C)C)c(NC(=O)/C=C/CN(C)CCCN2CCN(CCCON(C)C(=O)N3N=C(c4cc(F)ccc4F)S[C@@]3(CCCN)c3ccccc3)CC2)cc1Nc1nccc(-c2cn(C)c3ccccc23)n1]

General Information: Synthetic route of compound 10 is reported in CN107216313, 2017, A.

Step 1. A mixture of tert-butyl hydroxy(methyl)carbamate (7.5 g, 51.0 mmol, 1.0 equiv), 3-bromopropan-1-ol (8.5 g, 61.1 mmol, 5.5 mL, 1.2 equiv), Cs₂CO₃ (33.2 g, 102 mmol, 2.0 equiv) in DMF (75.0 mL) was degassed and purged with N2 for 3 times, and then the mixture was stirred at 70° C. for 20 h under N₂ atmosphere. The mixture was diluted with H₂O (150 mL) and extracted with EtOAc (80.0 mL×3). The combined organic layers were washed with brine (100 mL×2), dried over Na₂SO₄, filtered and concentrated under reduced pressure to give a residue. The residue was purified by flash silica gel chromatography (from PE/EtOAc=1/1 to 3/7) to give tert-butyl 3-hydroxypropoxy(methyl)carbamate (10.8 g, 47.5 mmol, 46% yield) as yellow oil. ¹H NMR (400 MHz, CDCl₃) δ=4.0 (t, J=5.6 Hz, 2H), 3.78 (t, J=5.6 Hz, 2H), 3.10 (s, 3H), 1.80 (m, 2H), 1.47 (s, 9H).

Step 2. To a solution of tert-butyl 3-hydroxypropoxy(methyl)carbamate (1.0 g, 4.4 mmol, 1.0 equiv) in DCM (10.0 mL) was added TFA (5.1 g, 45.1 mmol, 3.4 mL) and was stirred at 23° C. for 1 h. The mixture was concentrated to give the residue and used for the next step without purification. Compound 3-((methylamino)oxy)propan-1-ol (480 mg, TFA) was obtained as yellow oil.

Step 3. A mixture of 3-((methylamino)oxy)propan-1-ol (480 mg, 2.19 mmol, 1.0 equiv) 1-((S)-2-(3-((tert-butoxycarbonyl)amino)propyl)-5-(2,5-difluorophenyl)-2-phenyl-2,3-dihydro-1,3,4-thiadiazole-3-carbonyl)-1-methyl-¹H-imidazol-1-ium iodide (1.5 g, 2.2 mmol, 1.0 equiv), TEA (1.1 g, 10.9 mmol, 1.5 mL, 5.0 equiv) in THF (10.0 mL) was degassed and purged with N2 for 3 times, and then the mixture was stirred at 23° C. for 16 h under N₂ atmosphere. The mixture was concentrated to get the residue the residue was washed with brine and extracted with DCM (100 mL×2) and dried over Na₂SO₄. The mixture was concentrated to give the residue and purified by flash silica gel chromatography (from PE/EtOAc=3/1 to 1/1) to give (S)-tert-butyl (3-(5-(2,5-difluorophenyl)-3-((3-hydroxypropoxy)(methyl)carbamoyl)-2-phenyl-2,3-dihydro-1,3,4-thiadiazol-2-yl)propyl)carbamate (822 mg, 1.3 mmol, 59% yield) as colorless oil. ¹H NMR (400 MHz, CDCl₃) δ=7.50-7.45 (m, 3H), 7.39-7.34 (m, 2H), 7.30 (d, J=6.8 Hz, 1H), 7.11 (t, J=1.6, 6.4 Hz, 2H), 4.68 (s, 1H), 4.08 (s, 2H), 3.75-3.70 (m, 2H), 3.33-3.24 (m, 5H), 3.21-3.10 (m, 1H), 2.45-2.35 (m, 1H), 2.05 (s, 1H), 1.86-1.76 (m, 3H), 1.45 (s, 8H), 1.46-1.44 (m, 1H)

Step 4. To a solution of (S)-tert-butyl (3-(5-(2,5-difluorophenyl)-3-((3-hydroxypropoxy)(methyl)carbamoyl)-2-phenyl-2,3-dihydro-1,3,4-thiadiazol-2-yl)propyl) carbamate (100 mg, 159 μmol, 1.0 equiv) in DCM (5.0 mL) was added DMP (169 mg, 398 μmol, 2.5 equiv) at 25° C. for 1.5 h. The reaction mixture was diluted with EtOAc (100 mL) and Na₂SO₃ (40.0 mL) and stirred at 25° C. for 5 min. Then the mixture was extracted with NaHCO₃ (20.0 mL×2). The combined organic layers were dried over Na₂SO₄, filtered and concentrated under reduced pressure to give a residue. Compound (S)-2-butyl-5-(2,5-difluorophenyl)-N-methyl-N-(3-oxopropoxy)-2-phenyl-1,3,4-thiadiazole-3(2H)-carboxamide (160 mg, crude) was obtained as a white solid.

Step 5. To a solution of (S)-2-butyl-5-(2,5-difluorophenyl)-N-methyl-N-(3-oxopropoxy)-2-phenyl-1,3,4-thiadiazole-3(2H)-carboxamide (432 mg, 1.2 mmol, 1.5 equiv, TFA) in DCM (10.0 mL) and the pH was adjusted to 8 by progressively adding TEA (727 mg, 7.2 mmol, 1.0 mL, 9.2 equiv), then the pH was adjusted to around 5 by progressively adding AcOH (522 mg, 8.7 mmol, 0.5 mL, 11.2 equiv). Then the (E)-methyl 4-(methyl(3-(piperazin-1-yl)propyl)amino)but-2-enoate (439 mg, 780 μmol, crude purity, 1.0 equiv) was added to the mixture at 0° C. Then the solution was added NaBH(OAc)₃ (1.7 g, 7.8 mmol, 10.0 equiv) and stirred at 25° C. for 4 h. The reaction mixture was diluted with NH₄Cl (10.0 mL) and extracted with EtOAc (20.0 mL×2). The combined organic layers were dried over Na₂SO₄, filtered and concentrated under reduced pressure to give a residue. The crude product was purified by prep-HPLC: column: Phenomenex C18 150×25mmx10%m; mobile phase: [water(NH₄HCO₃)-ACN]; B%: 54%-84%, 5 min) to give the (E)-methyl 8-(4-(3-(((S)-2-(3-((tert-butoxycarbonyl)amino) propyl)-5-(2,5-difluorophenyl)-N-methyl-2-phenyl-2,3-dihydro-1,3,4-thiadiazole-3-carboxamido)oxy)propyl)piperazin-1-yl)-5-methyloct-2-enoate (128 mg, 86 μmol, 11% yield,) as a yellow solid. ¹H NMR (400 MHz, CDCl₃) δ=7.48 (d, J=8.2 Hz, 3H), 7.39-7.29 (m, 3H), 7.16-7.07 (m, 2H), 7.02-6.92 (m, 1H), 5.98 (d, J=16.0 Hz, 1H), 4.69 (d, J=1.6 Hz, 1H), 4.02 (d, J=2.4 Hz, 2H), 3.75 (s, 2H), 3.30-3.11 (m, 7H), 2.52-2.33 (m, 9H), 2.23 (s, 1H), 2.11-1.56 (m, 13H), 1.45 (s, 9H).

Step 6. To a solution of (E)-methyl 8-(4-(3-(((S)-2-(3-((tert-butoxycarbonyl)amino) propyl)-5-(2,5-difluorophenyl)-N-methyl-2-phenyl-2,3-dihydro-1,3,4-thiadiazole-3-carboxamido)oxy)propyl)piperazin-1-yl)-5-methyloct-2-enoate (128 mg, 87 μmol, 1.0 equiv) in THF (1.0 mL) and H₂O (0.3 mL) was added LiOH·H₂O (11 mg, 260 μmol, 3.0 equiv). The mixture was stirred at 50° C. for 1.5 h. Evaporate the solution on a water bath under reduced pressure using a rotary evaporator. The crude product was purified by prep-HPLC:column: Phenomenex C18 150×25mmx10%m; mobile phase: [water(NH4HCO₃)-ACN]; B%: 28%-58%, 8 min) to give the (S,E)-4-((3-(4-(3-((2-(3-((tert-butoxycarbonyl)amino)propyl)-5-(2,5-difluorophenyl)-N-methyl-2-phenyl-2,3-dihydro-1,3,4-thiadiazole-3-carboxamido)oxy)propyl)piperazin-1-yl)propyl)(methyl)amino)but-2-enoic acid (24 mg, 26 μmol, 30% yield) was obtained as a white solid. ¹H NMR (400 MHz, CDCl₃) δ=7.49-7.45 (m, 3H), 7.38-7.34 (m, 2H), 7.31-7.28 (m, 2H), 7.16-7.09 (m, 2H), 4.07-3.93 (m, 2H), 3.31-3.10 (m, 8H), 2.89-2.17 (m, 19H), 2.07-1.91 (m, 4H), 1.49-1.42 (m, 11H).

Step 7. To a solution of (S,E)-4-((3-(4-(3-((2-(3-((tert-butoxycarbonyl)amino)propyl)-5-(2,5-difluorophenyl)-N-methyl-2-phenyl-2,3-dihydro-1,3,4-thiadiazole-3-carboxamido)oxy)propyl)piperazin-1-yl)propyl)(methyl)amino)but-2-enoic acid (30 mg, 33 μmol, 1.0 equiv) and N-(2-(dimethylamino)ethyl)-5-methoxy-N-methyl-N4-(4-(1-methyl-1H-indol-3-yl)pyrimidin-2-yl)benzene-1,2,4-triamine (15 mg, 33 μmol) in DMF (0.5 mL) was added CMPI (85 mg, 332 μmol, 10.0 equiv) and TEA (16.8 mg, 166 μmol, 23 μL, 5.0 equiv). The mixture was stirred at 23° C. for 20 h. The mixture was poured into H₂O (10 mL) and extracted with DCM (10 mL×2) and combined organic layer was washed with brine (10 mL), concentrated to give the residue. Compound (S,E)-tert-butyl-(3-(5-(2,5-difluorophenyl)-3-((3-(4-(3-((4-((2-((2-(dimethyl amino)ethyl)(methyl)amino)-4-methoxy-5-((4-(1-methyl-¹H-indol-3-yl)pyrimidin-2-yl)amino)phenyl)amino)-4-oxobut-2-en-1-yl)(methyl)amino)propyl)piperazin-1-yl)propoxy) (methyl)carbamoyl)-2-phenyl-2,3-dihydro-1,3,4-thiadiazol-2-yl)propyl)carbamate (30 mg, 3 μmol) as yellow oil.

Step 8. A mixture of tert-butyl N-[3-[(2S)-5-(2,5-difluorophenyl)-3-[3-[4-[3-[[(E)-4-[2-[2-(dimethylamino)ethyl-methyl-amino]-4-methoxy-5-[ [4-(1-methylindol-3-yl)pyrimidin-2-yl]amino]anilino]-4-oxo-but-2-enyl]-methyl-amino]propyl]piperazin-1-yl]propoxy-methyl-carbamoyl]-2-phenyl-1,3,4-thiadiazol-2-yl]propyl]carbamate (30 mg, 25 μmol, 1.0 equiv) in DCM (3 mL), TFA (1 mL) was degassed and purged with N₂ for 3 times, and then the mixture was stirred at 25° C. for 1 h under N₂ atmosphere. The mixture was concentrated to give the residue and was purified by the prep-HPLC (column: Phenomenex luna C18 150*25 mm*10 m; mobile phase: [water(HCl)-ACN]; B%: 9%-39%, 10 min) to give the (2S)-2-(3-aminopropyl)-5-(2,5-difluorophenyl)-N-[3-[4-1[3-[[(E)-4-1[2-[2-(dimethylamino)ethyl-methyl-amino]-4-methoxy-5-1[[4-(1-methylindol-3-yl)pyrimidin-2-yl]amino]anilino]-4-oxo-but-2-enyl]-methyl-amino]propyl]piperazin-1-yl]propoxy]-N-methyl-2-phenyl-1,3,4-thiadiazole-3-carboxamide (9.6 mg, 7.3 μmol, 29% yield, 98% purity, 5HCl) as a yellow solid. LC-MS: MS (ES′): RT=2.107 min, m/z=1115.9 [M+H⁺]; LCMS Method: 05.¹H NMR (400 MHz, MeOD) δ=8.59 (s, 1H), 8.51-8.12 (m, 2H), 8.06-7.95 (m, 1H), 7.68 (d, J=7.6 Hz, 2H), 7.61-7.48 (m, 4H), 7.46-7.26 (m, 6H), 7.19 (d, J=15.2 Hz, 1H), 7.10 (s, 1H), 7.06-6.95 (m, 1H), 4.36-4.07 (m, 5H), 4.02-3.93 (m, 7H), 3.87-3.66 (m, 5H), 3.61-3.52 (m, 6H), 3.48-3.38 (m, 4H), 3.27-3.12 (m, 5H), 3.05-2.91 (m, 11H), 2.82 (s, 3H), 2.60-2.48 (m, 1H), 2.42-2.27 (m, 2H), 2.24-1.84 (m, 5H).

### Example 131—Synthesis of Compound I-147

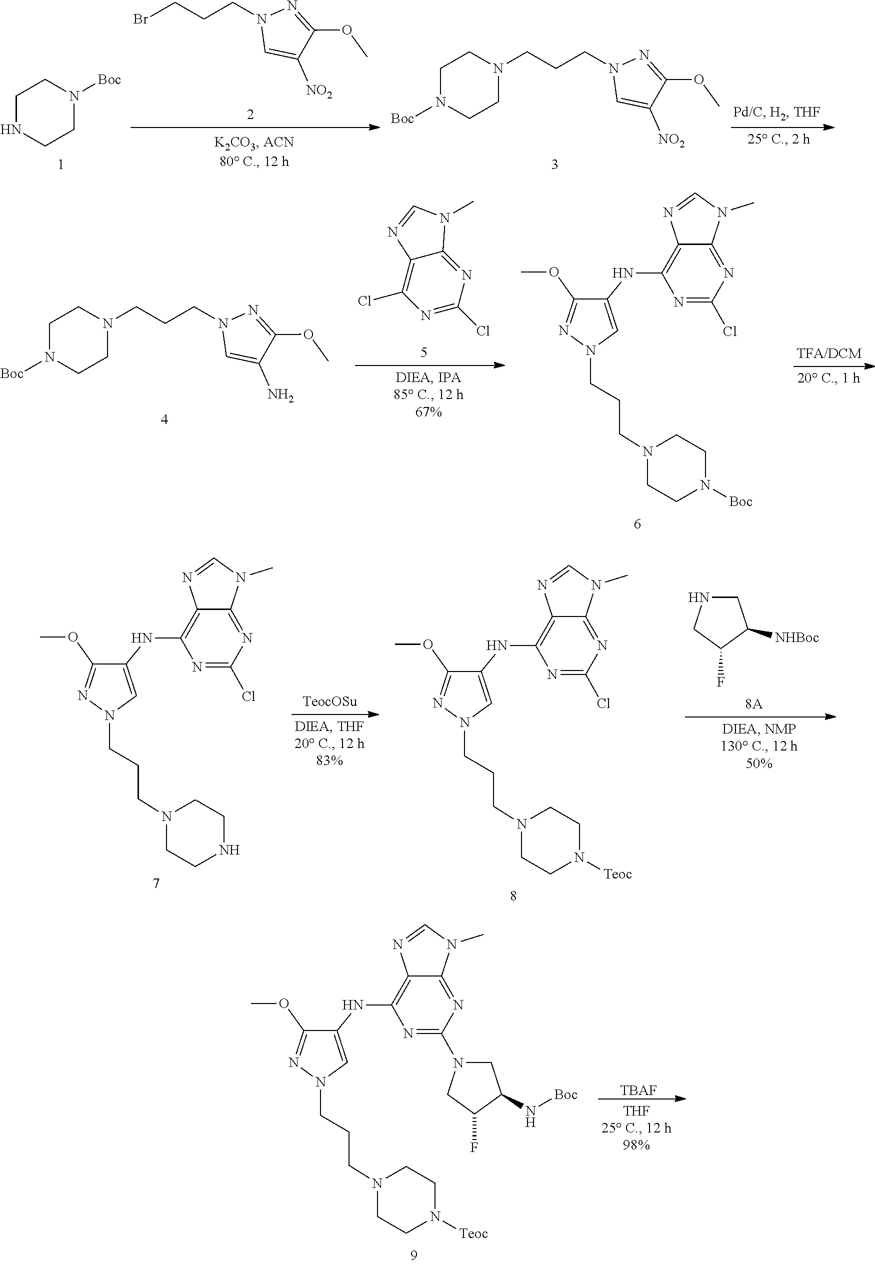

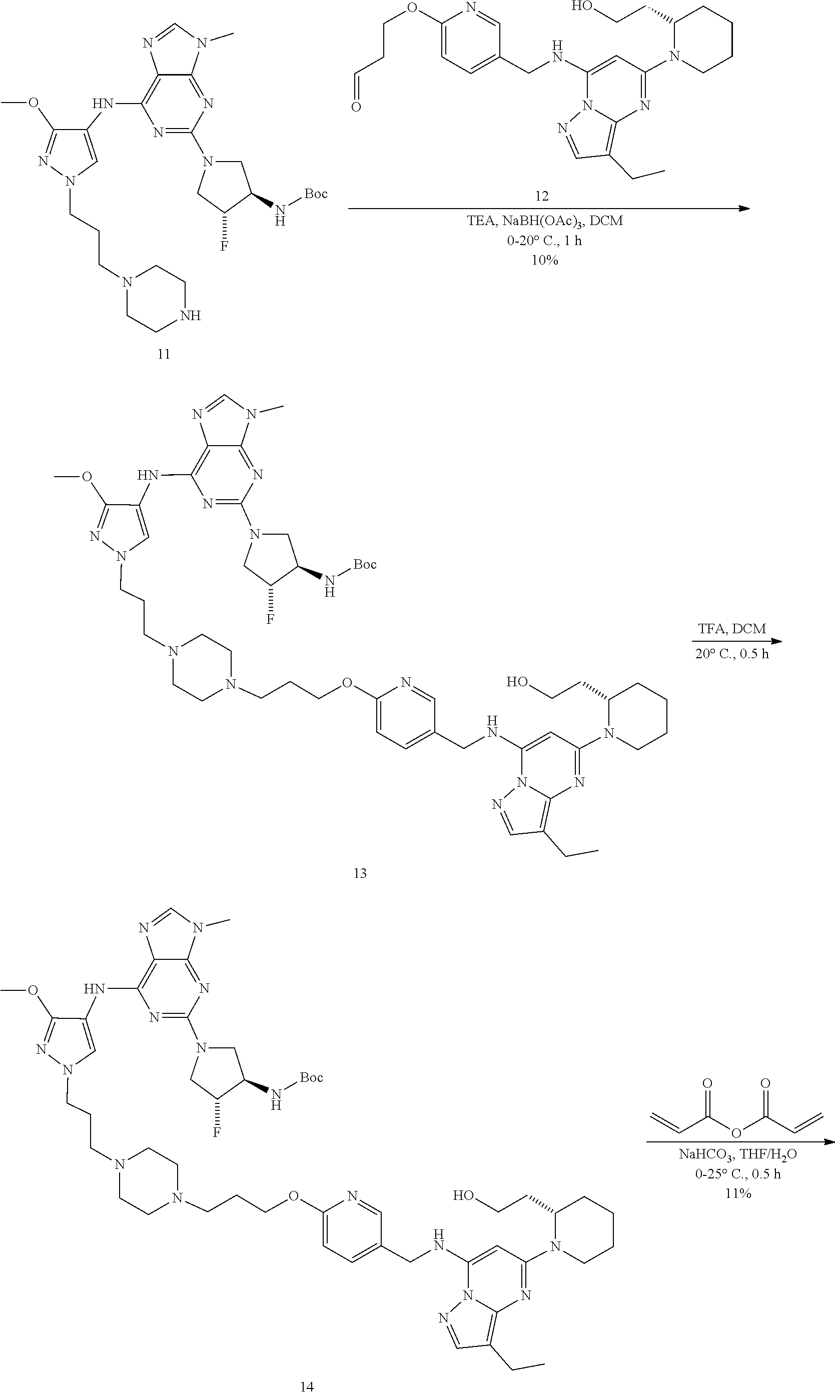

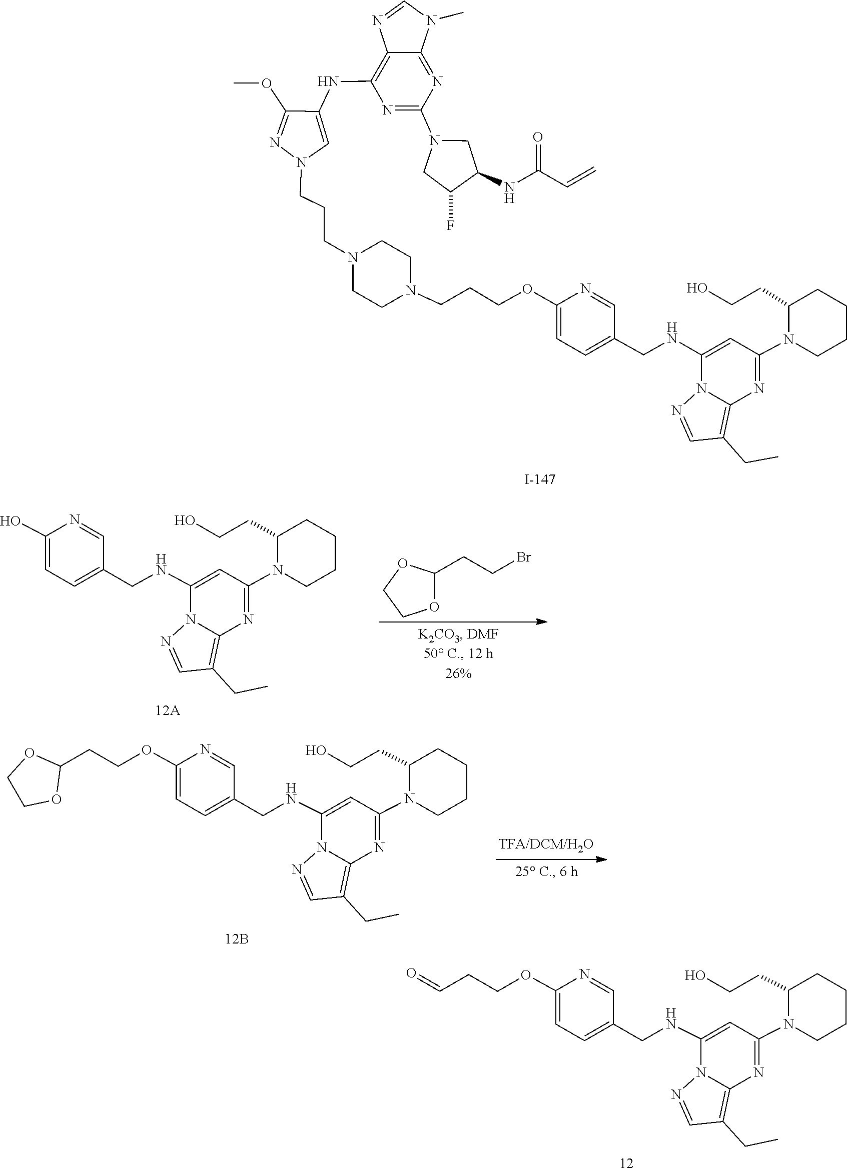 [SMILES: C=CC(=O)N[C@@H]1CN(c2nc(Nc3cn(CCCN4CCN(CCCOc5ccc(CNc6cc(N7CCCC[C@H]7CCO)nc7c(CC)cnn67)cn5)CC4)nc3OC)c3ncn(C)c3n2)C[C@H]1F ; CCc1cnn2c(NCc3ccc(O)nc3)cc(N3CCCC[C@H]3CCO)nc12 ; BrCCC1OCCO1 ; CCc1cnn2c(NCc3ccc(OCCC4OCCO4)nc3)cc(N3CCCC[C@H]3CCO)nc12 ; CCc1cnn2c(NCc3ccc(OCCC=O)nc3)cc(N3CCCC[C@H]3CCO)nc12]

Step 1. The mixture of 1-(3-bromopropyl)-3-methoxy-4-nitro-pyrazole (2.7 g, 10.2 mmol, 1.0 equiv) and K₂CO₃ (4.2 g, 30.7 mmol, 3.0 equiv) and tert-butyl piperazine-1-carboxylate (2.09 g, 11.2 mmol, 1.1 equiv) in ACN (40 mL) was stirred at 80° C. for 12 h. The mixture was concentrated and purified by column chromatography (SiO₂, Petroleum ether/Ethyl acetate=1/1 to 0/1) to give tert-butyl 4-[3-(3-methoxy-4-nitro-pyrazol-1-yl)propyl]piperazine-1-carboxylate (4 g, crude). ¹H NMR (400 MHz, CDCl₃): δ7.96 (s, 1H), 4.04-3.90 (m, 5H), 3.39 (s, 4H), 2.46-2.20 (m, 6H), 2.06-1.98 (m, 2H), 1.39 (s, 9H).

Step 2. To a solution of tert-butyl 4-[3-(3-methoxy-4-nitro-pyrazol-1-yl)propyl]piperazine-1-carboxylate (4 g, 11 mmol, 1.0 equiv) in THF (40 mL) was added Pd/C (0.5 g, 10% purity) under N2. The mixture was stirred under H₂ (15 psi) at 25° C. for 2 h. The mixture was filtered and concentrated to give tert-butyl 4-[3-(4-amino-3-methoxy-pyrazol-1-yl)propyl]piperazine-1-carboxylate (4 g, crude). ¹H NMR (400 MHz, CDCl₃): δ 6.91 (s, 1H), 3.98-3.88 (m, 4H), 3.83-3.71 (m, 1H), 3.51 (s, 4H), 2.56-2.29 (m, 6H), 2.10-1.95 (m, 2H), 1.48 (s, 9H).

Step 3. The mixture of tert-butyl 4-[3-(4-amino-3-methoxy-pyrazol-1-yl)propyl]piperazine-1-carboxylate (4 g, 12 mmol, 1.0 equiv) and 2,6-dichloro-9-methyl-purine (2.4 g, 11.8 mmol, 1.0 equiv) and DIEA (4.6 g, 35 mmol, 3.0 equiv) in IPA (50 mL) was stirred at 85° C. for 12 h. The solution was concentrated and purified by column chromatography (SiO₂, Petroleum ether/Ethyl acetate=1/1 to 0/1) to give tert-butyl 4-[3-[4-[(2-chloro-9-methyl-purin-6-yl)amino]-3-methoxy-pyrazol-1-yl]propyl]piperazine-1-carboxylate (4 g, 67% yield). ¹H NMR (400 MHz, CDCl₃): δ7.98 (s, 1H), 7.67 (s, 1H), 7.38 (s, 1H), 4.03-3.97 (m, 2H), 3.90 (s, 3H), 3.76 (s, 3H), 3.49-3.31 (m, 4H), 2.40-2.19 (m, 6H), 1.97-1.85 (m, 2H), 1.39 (s, 9H).

Step 4. The solution of tert-butyl 4-[3-[4-[(2-chloro-9-methyl-purin-6-yl)amino]-3-methoxy-pyrazol-1-yl]propyl]piperazine-1-carboxylate (2 g, 4.0 mmol, 1.0 equiv) in TFA (2 mL) and DCM (6 mL) was stirred at 20° C. for 1 h. The solution was concentrated to give 2-chloro-N-[3-methoxy-1-(3-piperazin-1-ylpropyl)pyrazol-4-yl]-9-methyl-purin-6-amine (2.05 g, crude, TFA salt) as brown oil.

Step 5. The solution of 2-chloro-N-[3-methoxy-1-(3-piperazin-1-ylpropyl)pyrazol-4-yl]-9-methyl-purin-6-amine (2.0 g, 3.9 mmol, 1.0 equiv, TFA salt) and (2,5-dioxopyrrolidin-1-yl) 2-trimethylsilylethyl carbonate (2.0 g, 7.8 mmol, 2.0 equiv) and DIEA (2.5 g, 19.7 mmol, 5.0 equiv) in THF (30 mL) was stirred at 20° C. for 12 h. The solution was concentrated and purified by column chromatography (SiO₂, Petroleum ether/Ethyl acetate=1/1 to 0/1) to give 2-trimethylsilylethyl 4-[3-1[4-[(2-chloro-9-methyl-purin-6-yl)amino]-3-methoxy-pyrazol-1-yl]propyl]piperazine-1-carboxylate (1.8 g, 83% yield) as a yellow oil. ¹H NMR (400 MHz, CDCl₃): δ 8.07 (s, 1H), 7.76 (s, 1H), 7.52-7.36 (m, 1H), 4.23-4.16 (m, 2H), 4.12-4.05 (m, 2H), 3.99 (s, 3H), 3.85 (s, 3H), 3.65-3.45 (m, 4H), 2.59-2.21 (m, 6H), 2.08-2.00 (m, 2H), 1.08-0.98 (m, 2H), 0.06 (s, 9H).

Step 6. The solution of 2-trimethylsilylethyl 4-[3-[4-[(2-chloro-9-methyl-purin-6-yl)amino]-3-methoxy-pyrazol-1-yl]propyl]piperazine-1-carboxylate (350 mg, 636 μmol, 1.0 equiv) and tert-butyl N-[(3R,4R)-4-fluoropyrrolidin-3-yl]carbamate (181 mg, 886 μmol, 1.4 equiv) and DIEA (371 mg, 2.87 mmol, 4.5 equiv) in NMP (4 mL) was stirred at 130° C. for 12 h. The solution was filtered and purified by prep-HPLC (column: Waters Xbridge C18 150*50 mm*10 m; mobile phase: [water(NH4HCO₃)-ACN]; B%: 47%-77%, 10 min) to give 2-trimethylsilylethyl 4-[3-1[4-1[[2-1[(3R,4R)-3-(tert-butoxycarbonylamino)-4-fluoro-pyrrolidin-1-yl]-9-methyl-purin-6-yl]amino]-3-methoxy-pyrazol-1-yl]propyl]piperazine-1-carboxylate (230 mg, 50% yield). ¹H NMR (400 MHz, CDCl₃): δ7.94 (s, 1H), 7.51 (s, 1H), 7.12 (s, 1H), 5.28-4.34 (m, 3H), 4.30-3.42 (m, 17H), 2.54-2.29 (m, 4H), 2.09-1.99 (m, 2H), 1.47 (s, 9H), 1.34-1.21 (m, 2H), 1.09-0.81 (m, 3H), 0.075-0.05 (m, 9H).

Step 7. The solution of 2-trimethylsilylethyl 4-[3-[4-[[2-[(3R,4R)-3-(tert-butoxy carbonylamino)-4-fluoro-pyrrolidin-1-yl]-9-methyl-purin-6-yl]amino]-3-methoxy-pyrazol-1-yl]propyl]piperazine-1-carboxylate (230 mg, 320 μmol, 1.0 equiv) in THF (2 mL) and TBAF (1 M, 2 mL, 6.2 equiv) was stirred at 25° C. for 12 h. The solution was concentrated and purified by prep-HPLC (column: Waters Xbridge 150*25 mm*5 m; mobile phase: [water(NH₄HCO₃)-ACN]; B%: 26%-56%, 8 min and column: Waters Xbridge 150*25 mm*5 m; mobile phase: [water(NH4HCO₃)-ACN]; B%: 28%-58%, 8 min) to give tert-butyl N-[(3R,4R)-4-fluoro-1-[6-[[3-methoxy-1-(3-piperazin-1-ylpropyl)pyrazol-4-yl]amino]-9-methyl-purin-2-yl]pyrrolidin-3-yl]carbamate (180 mg, 98% yield). ¹H NMR (400 MHz, CDCl₃): δ8.01-7.89 (m, 1H), 7.55-7.45 (m, 1H), 4.46-4.30 (m, 1H), 4.15-3.89 (m, 9H), 3.86-3.65 (m, 5H), 3.63-3.47 (m, 5H), 2.95-2.85 (m, 2H), 2.50-2.31 (m, 5H), 2.13-2.00 (m, 2H), 1.45 (s, 9H).

Step 8. The mixture of 2-(2-bromoethyl)-1,3-dioxolane (171 mg, 946 μmol, 113 μL, 1.5 equiv) and 5-[[[3-ethyl-5-[(2S)-2-(2-hydroxyethyl)-1-piperidyl]pyrazolo[1,5-a]pyrimidin-7-yl]amino]methyl]pyridin-2-ol (250 mg, 630 μmol, 1.0 equiv) and K₂CO₃ (261 mg, 1.9 mmol, 3.0 equiv) in DMF (2 mL) was stirred at 50° C. for 12 h. The mixture was filtered to get the filtrate, which was purified by prep-HPLC (column: Waters Xbridge C18 150*50 mm*10 m; mobile phase: [water(NH4HCO₃)-ACN]; B%: 39%-69%, 10 min) to give 2-[(2S)-1-[7-[[6-[2-(1,3-dioxolan-2-yl)ethoxy]-3-pyridyl]methylamino]-3-ethyl-pyrazolo[1,5-a]pyrimidin-5-yl]-2-piperidyl]ethanol (80 mg, 26% yield) as a white solid. ¹H NMR (400 MHz, CDCl₃): δ8.25-8.10 (m, 1H), 7.76-7.55 (m, 2H), 6.88-6.50 (m, 2H), 5.46-5.27 (m, 1H), 5.20-4.95 (m, 2H), 4.63-4.36 (m, 4H), 4.10-3.57 (m, 6H), 3.47-3.34 (m, 1H), 3.21-3.00 (m, 1H), 2.77-2.55 (m, 2H), 2.30-2.01 (m, 4H), 1.84-1.69 (m, 7H), 1.31-1.24 (m, 3H).

Step 9. The solution of 2-[(2S)-1-[7-[[6-[2-(1,3-dioxolan-2-yl)ethoxy]-3-pyridyl]methylamino]-3-ethyl-pyrazolo[1,5-a]pyrimidin-5-yl]-2-piperidyl]ethanol (50 mg, 101 μmol, 1.0 equiv) in DCM (0.5 mL) and TFA (100 μL) and H₂O (10 mg, 574 μmol, 5.7 equiv) was stirred at 25° C. for 6 h. The solution was used for next step directly.

Step 10. To the solution of tert-butyl N-[(3R,4R)-4-fluoro-1-[6-[[3-methoxy-1-(3-piperazin-1-ylpropyl)pyrazol-4-yl]amino]-9-methyl-purin-2-yl]pyrrolidin-3-yl]carbamate (70 mg, 122 μmol, 1.23 equiv) and TEA (218 mg, 2.16 mmol, 21.7 equiv) and NaBH(OAc)₃ (105 mg, 497 μmol, 5.0 equiv) in DCM (0.5 mL) was added the solution of 3-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]propanal (45 mg, 99 μmol, 1.0 equiv) in DCM (0.5 mL) at 0° C., then the solution was stirred at 20° C. for 1 h. The solution was concentrated and purified by prep-TLC(SiO₂, Dichloromethane: Methanol=10:1) to give tert-butyl N-[(3R,4R)-1-[6-[[1-[3-[4-[3-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]propyl]piperazin-1-yl]propyl]-3-methoxy-pyrazol-4-yl]amino]-9-methyl-purin-2-yl]-4-fluoro-pyrrolidin-3-yl]carbamate (10 mg, 10% yield).

Step 11. The solution of tert-butyl N-[(3R,4R)-1-[6-[[1-[3-[4-[3-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]propyl]piperazin-1-yl]propyl]-3-methoxy-pyrazol-4-yl]amino]-9-methyl-purin-2-yl]-4-fluoro-pyrrolidin-3-yl]carbamate (10 mg, 9.9 μmol, 1.0 equiv) in TFA (0.3 mL) and DCM (1.5 mL) was stirred at 20° C. for 0.5 h. The solution was concentrated to give 2-[(2S)-1-[7-[[6-[3-[4-[3-[4-[[2-[(3R,4R)-3-amino-4-fluoro-pyrrolidin-1-yl]-9-methyl-purin-6-yl]amino]-3-methoxy-pyrazol-1-yl]propyl]piperazin-1-yl]propoxy]-3-pyridyl]methylamino]-3-ethyl-pyrazolo[1,5-a]pyrimidin-5-yl]-2-piperidyl]ethanol (11 mg, crude, TFA salt) as colorless oil.

Step 12. To a solution of 2-[(2S)-1-[7-[[6-[3-[4-[3-[4-[[2-[(3R,4R)-3-amino-4-fluoro-pyrrolidin-1-yl]-9-methyl-purin-6-yl]amino]-3-methoxy-pyrazol-1-yl]propyl]piperazin-1-yl]propoxy]-3-pyridyl]methylamino]-3-ethyl-pyrazolo[1,5-a]pyrimidin-5-yl]-2-piperidyl]ethanol (11 mg, 11 μmol, 1.0 equiv, TFA salt) in THF (1 mL) and NaHCO₃ (2.16 g, 25.7 mmol, 2394 equiv) was added prop-2-enoyl prop-2-enoate (2.0 mg, 16 μmol, 1.5 equiv) at 0° C., then the solution was stirred at 25° C. for 0.5 h. The solution was concentrated and purified by prep-HPLC (column: Phenomenex C18 75*30 mm*3 m; mobile phase: [water(FA)-ACN]; B%: 10%-40%, 7 min) to give N-[(3R,4R)-1-[6-[[1-[3-[4-[3-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]propyl]piperazin-1-yl]propyl]-3-methoxy-pyrazol-4-yl]amino]-9-methyl-purin-2-yl]-4-fluoro-pyrrolidin-3-yl]prop-2-enamide (1.25 mg, 11% yield). ¹H NMR (400 MHz, MeOD): δ 8.60-8.45 (m, 1H), 8.23-8.12 (m, 1H), 8.10-8.05 (m, 1H), 7.82-7.73 (m, 2H), 7.64 (s, 1H), 6.85-6.75 (m, 1H), 6.33-6.19 (m, 2H), 5.70-5.63 (m, 1H), 5.51 (s, 1H), 5.38-5.09 (m, 2H), 4.54 (s, 2H), 4.37-4.27 (m, 2H), 4.11-3.80 (m, 9H), 3.71 (s, 3H), 3.58-3.49 (m, 1H), 3.08-2.93 (m, 1H), 2.76-2.34 (m, 12H), 2.15-1.92 (m, 6H), 1.77-1.60 (m, 6H), 1.56-1.41 (m, 1H), 1.34-1.27 (m, 4H), 1.25-1.19 (m, 3H) LC-MS: MS (ES′): RT=1.998 min, m/z=964.3 [M+H⁺]; LCMS method: 10.

### Example 132—Synthesis of Compound I-148

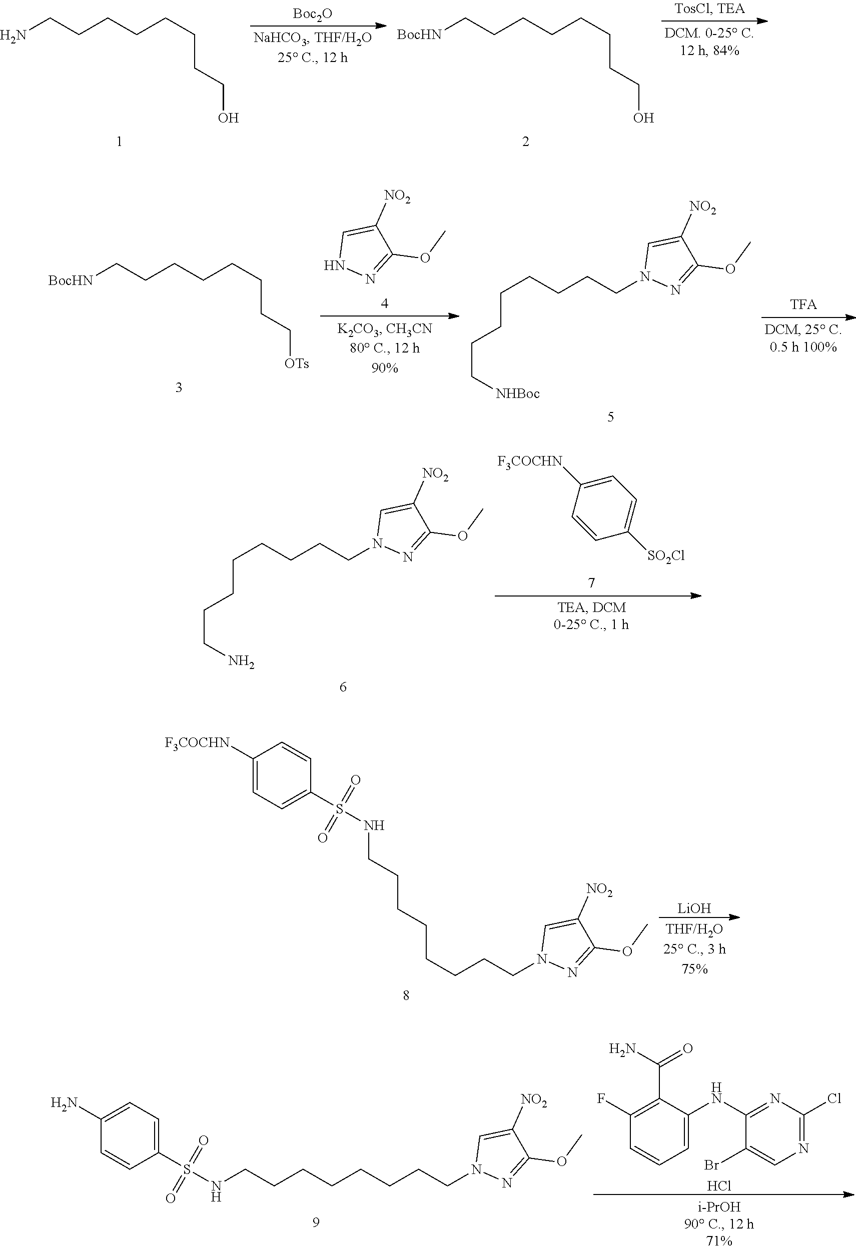

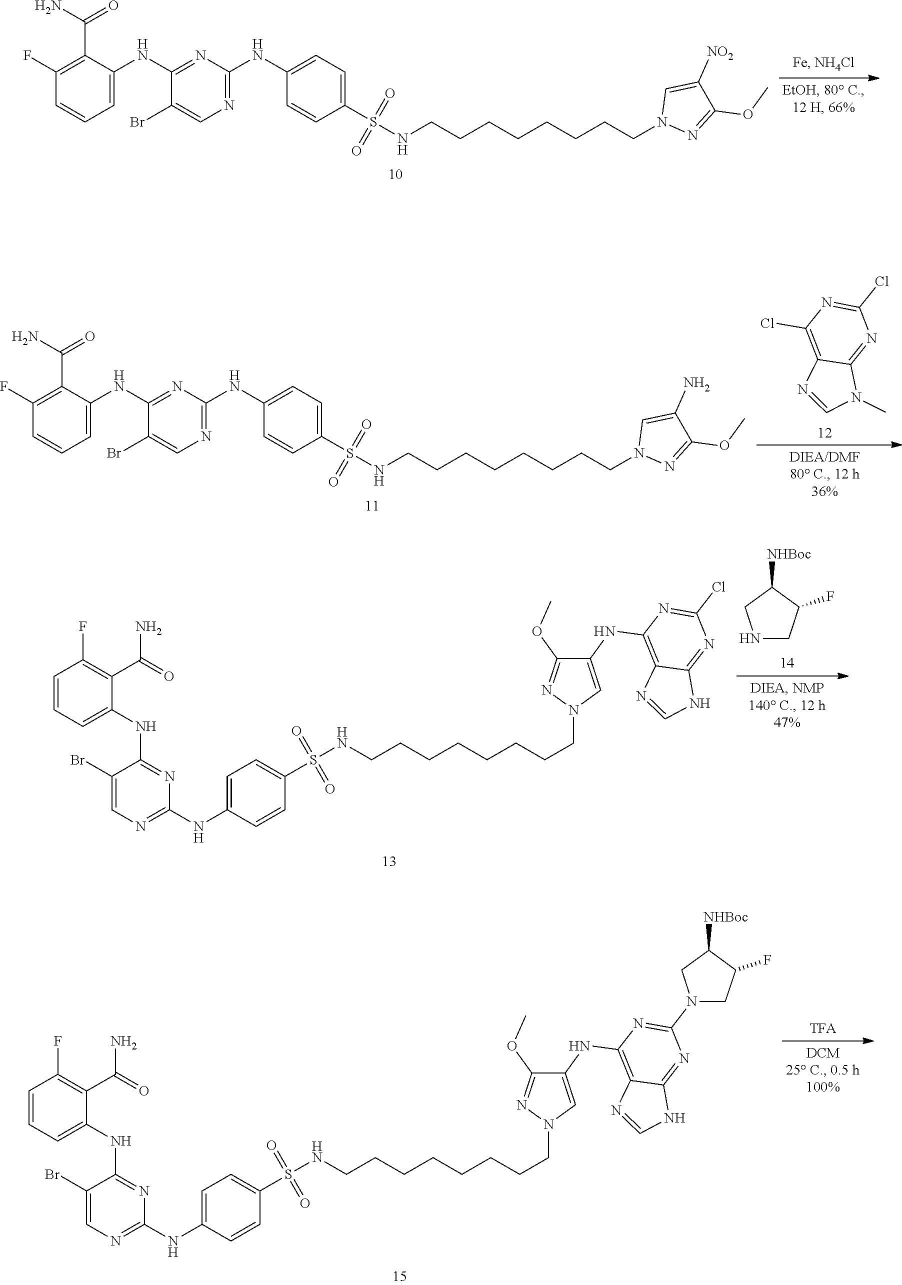

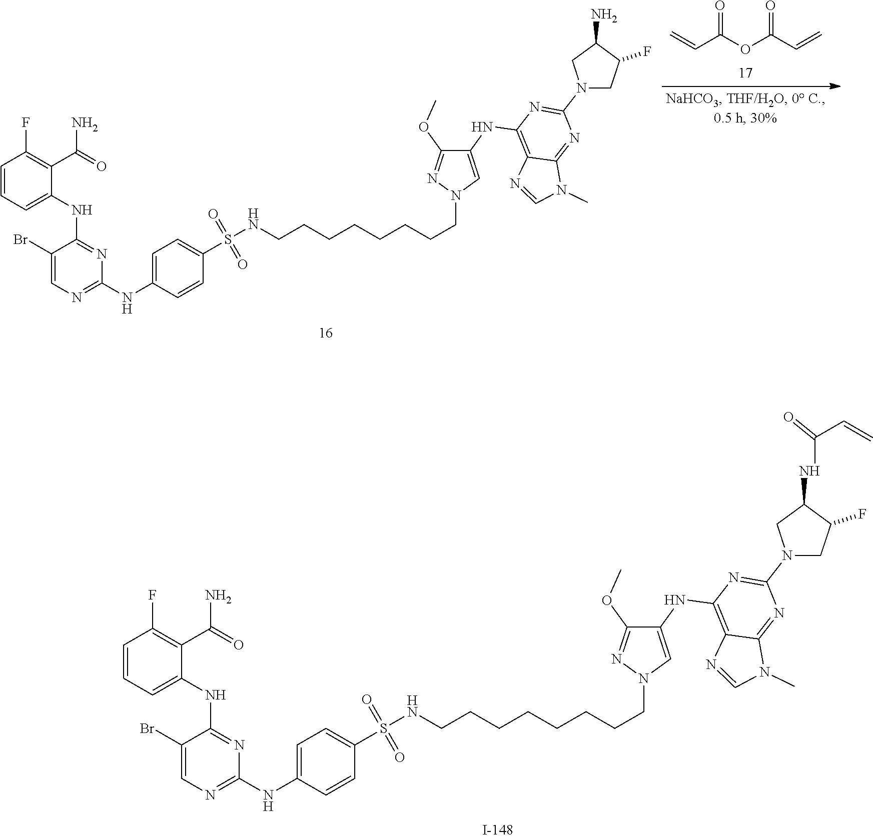

General Information: Compound 7 is a known compound from WO2020/206137, 2020, A1.

Step 1. To a solution of 8-aminooctan-1-ol (4 g, 27 mmol, 1.0 equiv) in THF (80 mL) and H₂O (40 mL) was added NaHCO₃ (3.0 g) and Boc₂0 (9.02 g, 41.3 mmol, 9.49 mL, 1.5 equiv), then it was stirred at 25° C. for 12 h The reaction mixture was diluted with 50 mL water and extracted with EA (100 mL×2). The combined organic layers was dried by anhydrous sodium sulfate, filtered and concentrated under reduced pressure to give a crude product. tert-butyl N-(8-hydroxyoctyl)carbamate (10.5 g, crude) was obtained as a colorless liquid and used for next step reaction

Step 2. To a solution of tert-butyl N-(8-hydroxyoctyl)carbamate (6.79 g, 27.6 mmol, 1.0 equiv) in DCM (150 mL) was added TEA (5.60 g, 55.3 mmol, 7.70 mL, 2.0 equiv) and 4-methylbenzenesulfonyl chloride (10.55 g, 55.35 mmol, 2.0 equiv) at 0° C., and then it was stirred at 25° C. for 12 h. The residue was diluted with 50 mL water and extracted with EtOAc (100 mL×2). The combined organic layers was dried by anhydrous Na₂SO₄, filtered and concentrated. The crude product was purified by column chromatography (SiO₂, PE:EA=10:1-5:1). 8-(tert-butoxycarbonylamino)octyl 4-methylbenzenesulfonate (9.32 g, 23.3 mmol, 84% yield) was obtained as a light yellow oil ¹H NMR (400 MHz, CDCl₃) δ7.80 (d, J=8.3 Hz, 2H), 7.35 (d, J=8.1 Hz, 2H), 4.49 (m, 1H), 4.02 (t, J=6.5 Hz, 2H), 3.20-2.97 (m, 2H), 2.46 (s, 3H), 1.68-1.59 (m, 2H), 1.57 (s, 2H), 1.46-1.45 (m, 6H), 1.35-1.14 (m, 9H).

Step 3. To a solution of 8-(tert-butoxycarbonylamino)octyl 4-methylbenzenesulfonate (3 g, 7 mmol, 1.0 equiv) in CH₃CN (40 mL) was added 3-methoxy-4-nitro-1H-pyrazole (1.07 g, 7.51 mmol, 1.0 equiv), K₂CO₃ (2.08 g, 15.0 mmol, 2.0 equiv), it was stirred at 80° C. for 12 h. The reaction mixture was filtered to remove K₂CO₃, the filtrate was added silica gel and concentrated. The crude product was purified by column chromatography (SiO₂, PE:EtOAc=3:1). tert-butyl N-[8-(3-methoxy-4-nitro-pyrazol-1-yl)octyl]carbamate (2.52 g, 6.80 mmol, 90% yield) was obtained as a light yellow oil. ¹H NMR (400 MHz, CDCl₃) δ7.99 (s, 1H), 4.56-4.43 (m, 1H), 4.05 (s, 3H), 3.96 (t, J=7.2 Hz, 2H), 3.18-3.03 (m, 2H), 1.90-1.80 (m, 2H), 1.45 (s, 9H), 1.37-1.22 (m, 1OH).

Step 4. To a solution of tert-butyl N-[8-(3-methoxy-4-nitro-pyrazol-1-yl) octyl]carbamate (1.5 g, 4.0 mmol, 1.0 equiv) in DCM (6 mL) was added TFA (3 mL), it was stirred at 25° C. for 0.5 h, The reaction mixture was concentrated to afford a TFA salt product. 8-(3-methoxy-4-nitro-pyrazol-1-yl)octan-1-amine (1.56 g, 4.06 mmol, 100% yield, TFA salt) was obtained as a light yellow liquid and used for next step reaction directly.

Step 5. To a solution of 8-(3-methoxy-4-nitro-pyrazol-1-yl)octan-1-amine (1.56 g, 4.06 mmol, 1.0 equiv, TFA salt) in DCM (10 mL) was added TEA (1.23 g, 12.1 mmol, 1.69 mL, 3.0 equiv) and 4-[(2,2,2-trifluoroacetyl)amino]benzenesulfonyl chloride (1.17 g, 4.06 mmol, 1.0 equiv) at 0° C., it was stirred at 25° C. for 1 h. The residue was diluted with 20 mL water and extracted with EA (50 mL×2). The combined organic layers were dried over Na₂SO₄, filtered and concentrated under reduced pressure to give a residue 2,2,2-trifluoro-N-[4-[8-(3-methoxy-4-nitro-pyrazol-1-yl)octylsulfamoyl]phenyl]acetamide (2.12 g, crude) was obtained as a light yellow oil and used for next step reaction directly.

Step 6. To a solution of 2,2,2-trifluoro-N-[4-[8-(3-methoxy-4-nitro-pyrazol-1-yl)octylsulfamoyl]phenyl]acetamide (2.12 g, 4.07 mmol, 1.0 equiv) in THF (6 mL) and H₂O (6 mL) was added LiOH.H₂O (341 mg, 8.13 mmol, 2.0 equiv), and then it was stirred at 25° C. for 3 h. The residue was diluted with 30 mL water and extracted with EtOAc (50 mL×2). The combined organic layers were dried over Na₂SO₄, filtered and concentrated under reduced pressure to give a residue. The residue was purified by column chromatography (SiO₂, Petroleum ether/Ethyl acetate=2/1 to 0/1). 4-amino-N-[8-(3-methoxy-4-nitro-pyrazol-1-yl)octyl]benzenesulfonamide (1.3 g, 3.0 mmol, 75% yield) was obtained as a yellow solid.

Step 7. To a solution of 4-amino-N-[8-(3-methoxy-4-nitro-pyrazol-1-yl)octyl]benzenesulfonamide (1 g, 2 mmol, 1.0 equiv) in i-PrOH (20 mL) was add HCl (59.0 mg, 2.35 μmol, 57.9 μL, 0.001 equiv) and 2-[(5-bromo-2-chloro-pyrimidin-4-yl)amino]-6-fluoro-benzamide (812 mg, 2.35 mmol, 1.0 equiv), and then it was stirred at 90° C. for 12 h. The reaction mixture was filtered to get a residue, and it was washed by i-PrOH (20 mL×3). 2-[[5-bromo-2-[4-[8-(3-methoxy-4-nitro-pyrazol-1-yl)octylsulfamoyl]anilino]pyrimidin-4-yl]amino]-6-fluoro-benzamide (1.23 g, 1.67 mmol, 71% yield) was obtained as a white solid and used for next step reaction directly. ¹H NMR (400 MHz, CD₃OD) δ8.34 (d, J=12.3 Hz, 2H), 7.99 (d, J=8.3 Hz, 1H), 7.79 (d, J=8.6 Hz, 2H), 7.64 (d, J=8.7 Hz, 2H), 7.51-7.41 (m, 1H), 7.25-7.09 (m, 1H), 4.04-3.85 (m, 5H), 2.84 (t, J=7.0 Hz, 2H), 1.89-1.74 (m, 2H), 1.49-1.38 (m, 2H), 1.27 (m, 8H).

Step 8. To a mixture of 2-[[5-bromo-2-[4-[8-(3-methoxy-4-nitro-pyrazol-1-yl)octylsulfamoyl]anilino]pyrimidin-4-yl]amino]-6-fluoro-benzamide (1.23 g, 1.67 mmol, 1.0 equiv) in NH₄Cl (6 mL) EtOH (12 mL) was added Fe (467 mg, 8.37 mmol, 5.0 equiv), and then it was stirred at 80° C. for 12 h. The reaction mixture was filtered and the residue was washed by THF (20 mL×3) to get a filtrate, the filtrate was diluted with 50 mL water and extracted with EtOAc (150 mL×2). The combined organic layers were dried over Na₂SO₄, filtered and concentrated under reduced pressure to give a residue. 2-[[2-[4-[8-(4-amino-3-methoxy-pyrazol-1-yl)octylsulfamoyl]anilino]-5-bromo-pyrimidin-4-yl]amino]-6-fluoro-benzamide (780 mg, 1.11 mmol, 66% yield) was obtained as a brown solid and used for next step reaction directly.

Step 9. To a solution of 2-[[2-[4-[8-(4-amino-3-methoxy-pyrazol-1-yl)octylsulfamoyl]anilino]-5-bromo-pyrimidin-4-yl]amino]-6-fluoro-benzamide (530 mg, 752 μmol, 1.0 equiv) and 2,6-dichloro-9-methyl-purine (152.72 mg, 752.19 μmol, 1.0 equiv) in DMF (6 mL) was added DIEA (291 mg, 2.26 mmol, 393 μL, 3.0 equiv), and then it was stirred at 80° C. for 12 h. The crude product was purified by prep-HPLC(column: Phenomenex luna C18 150*40 mm*15 m; mobile phase: [water(FA)-ACN]; B%: 48%-78%, 10 min). 2-[[5-bromo-2-[4-[8-[4-[(2-chloro-9-methyl-purin-6-yl)amino]-3-methoxy-pyrazol-1-yl]octylsulfamoyl]anilino]pyrimidin-4-yl]amino]-6-fluoro-benzamide (240 mg, 275 μmol, 36.% yield) was obtained as a brown solid.

Step 10. To a solution of 2-[[5-bromo-2-[4-[8-[4-[(2-chloro-9-methyl-purin-6-yl)amino]-3-methoxy-pyrazol-1-yl]octylsulfamoyl]anilino]pyrimidin-4-yl]amino]-6-fluoro-benzamide (142.18 mg, 163.21 μmol, 1.0 equiv) in NMP (1.5 mL) was added DIEA (63.28 mg, 489.6 μmol, 85.28 μL, 3.0 equiv) and tert-butyl N-[(3R,4R)-4-fluoropyrrolidin-3-yl]carbamate (50.0 mg, 244. μmol, 1.5 equiv), and then it was stirred at 140° C. for 12 h. The reaction mixture was purified by prep-HPLC (column: Waters Xbridge 150*25 mm*5 m; mobile phase: [water(NH4HCO₃)-ACN]; B%: 53%-83%, 8 min) to afford tert-butyl N-[(3R,4R)-1-[6-[[1-[8-[[4-[[5-bromo-4-(2-carbamoyl-3-fluoro-anilino)pyrimidin-2-yl]amino]phenyl]sulfonylamino]octyl]-3-methoxy-pyrazol-4-yl]amino]-9-methyl-purin-2-yl]-4-fluoro-pyrrolidin-3-yl]carbamate (80 mg, 77 μmol, 47% yield) as a yellow solid.

Step 11. To a solution of tert-butyl N-[(3R,4R)-1-[6-[[1-[8-[[4-[[5-bromo-4-(2-carbamoyl-3-fluoro-anilino)pyrimidin-2-yl]amino]phenyl]sulfonylamino]octyl]-3-methoxy-pyrazol-4-yl]amino]-9-methyl-purin-2-yl]-4-fluoro-pyrrolidin-3-yl]carbamate (80 mg, 77. μmol, 1.0 equiv) in DCM (1 mL) was added TFA (0.5 mL), and then it was stirred at 25° C. for 0.5 h. The reaction mixture was concentrated to afford crude product. 2-[[2-[4-[8-[4-[[2-[(3R,4R)-3-amino-4-fluoro-pyrrolidin-1-yl]-9-methyl-purin-6-yl]amino]-3-methoxy-pyrazol-1-yl]octylsulfamoyl]anilino]-5-bromo-pyrimidin-4-yl]amino]-6-fluoro-benzamide (81.07 mg, 77.00 μmol, 100% yield, TFA salt) was obtained as a light yellow oil and used for next step reaction directly.

Step 12. To a solution of 2-[[2-[4-[8-[4-[[2-[(3R,4R)-3-amino-4-fluoro-pyrrolidin-1-yl]-9-methyl-purin-6-yl]amino]-3-methoxy-pyrazol-1-yl]octylsulfamoyl]anilino]-5-bromo-pyrimidin-4-yl]amino]-6-fluoro-benzamide (80 mg, 75 μmol, 1.0 equiv, TFA salt) in THF (1 mL) and sat. NaHCO₃ (1 mL) was added prop-2-enoyl prop-2-enoate (9.58 mg, 75.9 μmol, 1.0 equiv) at 0° C., and then it was stirred at 0° C. for 0. 5 h. The reaction mixture was diluted with 10 mL water and extracted with EA (20 mL×3). The combined organic layers were dried over anhydrous Na₂SO₄, filtered and concentrated under reduced pressure to give a residue. The crude product was purified by prep-HPLC (column: Phenomenex Synergi C18 150*25 mm*10 μm; mobile phase: [water(FA)-ACN]; B%: 46%-76%, 10 min). 2-[[5-bromo-2-[4-[8-[4-[[2-[(3R,4R)-3-fluoro-4-(prop-2-enoylamino)pyrrolidin-1-yl]-9-methyl-purin-6-yl]amino]-3-methoxy-pyrazol-1-yl]octylsulfamoyl]anilino]pyrimidin-4-yl]amino]-6-fluoro-benzamide (23 mg, 23 μmol, 30% yield) was obtained as a white solid. ¹H NMR (400 MHz, DMSO-d₆) δ10.12 (s, 1H), 9.93 (s, 1H), 8.45 (m, 1H), 8.38 (s, 1H), 8.23 (m, J=8.3 Hz, 1H), 8.17-8.06 (m, 2H), 7.89-7.81 (m, 3H), 7.62 (d, J=8.8 Hz, 2H), 7.55-7.45 (m, 1H), 7.33 (t, J=5.9 Hz, 1H), 7.08 (t, J=9.4 Hz, 1H), 6.26-6.03 (m, 2H), 5.69-5.50 (m, 1H), 5.22-4.98 (m, 1H), 4.53-4.36 (m, 1H), 3.90 (m, J=6.7 Hz, 2H), 3.83-3.73 (m, 5H), 3.72-3.64 (m, 1H), 3.63-3.59 (m, 3H), 2.68-2.66 (m, 2H), 2.52 (m, 3H), 1.72-1.63 (m, 2H), 1.35-1.27 (m, 2H), 1.24-1.06 (m, 8H) LC-MS: MS (ES⁺): RT=0.886 min, m/z=994.3 [M+H⁺]; LCMS Method: 25.

### Example 133—Synthesis of Compound I-149

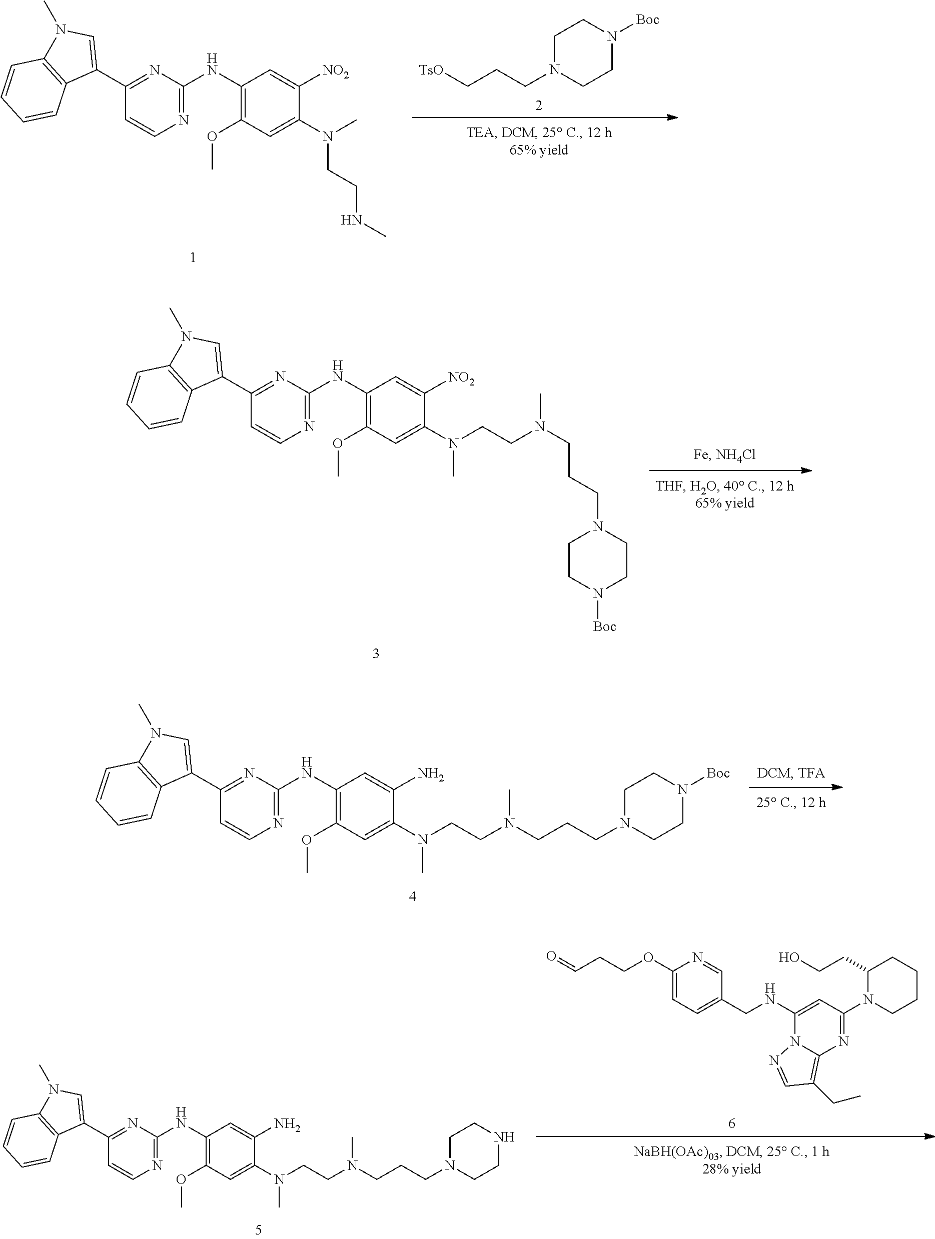

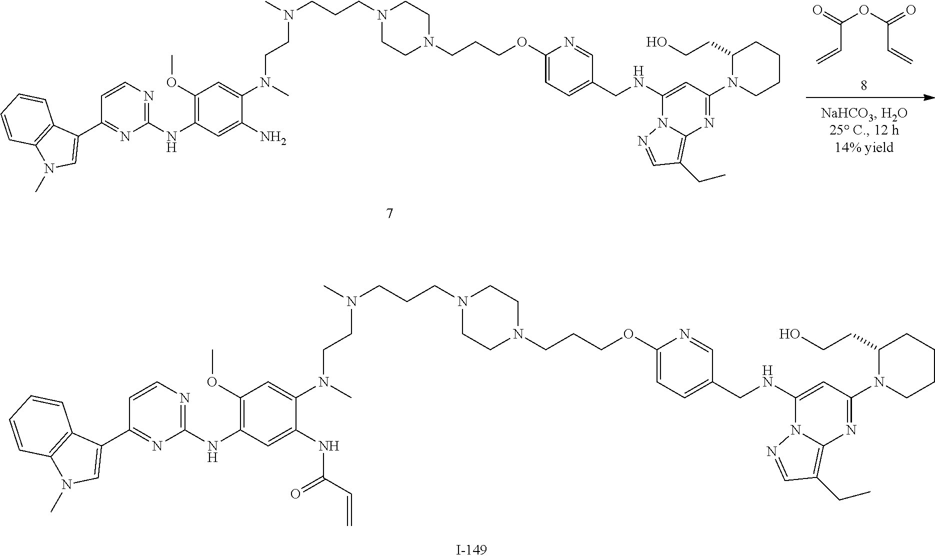

General Information: Synthetic route of compound 1 is reported in WO2018/119441, 2018, A1. Synthetic route of compound 2 is reported in WO2020/41331, 2020, A1.

Step 1. To a solution of 2-methoxy-N4-methyl-N4-[2-(methylamino)ethyl]-N1-[4-(1-methylindol-3-yl)pyrimidin-2-yl]-5-nitro-benzene-1,4-diamine (1.0 g, 2.2 mmol, 1.0 equiv) in DCM (10 mL) was added TEA (438 mg, 4.3 mmol, 2.0 equiv) and tert-butyl 4-[3-(p-tolylsulfonyloxy)propyl]piperazine-1-carboxylate (777 mg, 2.0 mmol, 0.9 equiv). The mixture was stirred at 25° C. for 12 h. The mixture was concentrated to give the residue. The residue was purified by the prep-HPLC (column: Phenomenex C18 250*50 mm*l0 m; mobile phase: [water(NH₄HCO₃)-ACN]; B%: 53%-83%, 8 min) to give the tert-butyl 4-[3-[2-[5-methoxy-N-methyl-4-[ [4-(1-methylindol-3-yl)pyrimidin-2-yl]amino]-2-nitro-anilino]ethylmethyl-amino]propyl]piperazine-1-carboxylate (971 mg, 1.4 mmol, 65% yield) as a white solid. ¹H NMR (400 MHz, DMSO-d₆) δ=8.65 (s, 1H), 8.36-8.33 (m, 1H), 8.33-8.32 (m, 1H), 8.36 (s, 1H), 8.06 (s, 1H), 3.96 (s, 3H), 3.88 (s, 3H), 3.29-3.28 (m, 2H), 3.25-3.24 (m, 2H), 3.20-3.16 (m, 2H), 2.80 (m, 3H), 2.68-2.67 (m, 2H), 2.21-2.15 (m, 11H), 1.36 (s, 9H).

Step 2. To a solution of tert-butyl 4-[3-[2-[5-methoxy-N-methyl-4-[[4-(1-methylindol-3-yl)pyrimidin-2-yl]amino]-2-nitro-anilino]ethylmethyl-amino]propyl]piperazine-1-carboxylate (490 mg, 712 μmol, 1.0 equiv) in EtOH (15 mL), H₂O (5 mL) and THF (1 mL) was added Fe (397 mg, 7.1 mmol, 10.0 equiv), NH₄Cl (381 mg, 7.1 mmol, 10.0 equiv). The mixture was stirred at 40° C. for 12 h. The mixture was filtered and concentrated to give the residue. The residue was purified by the prep-HPLC(column: Phenomenex luna C18 250*50 mm*10 m; mobile phase: [water(HCl)-ACN]; B%: 8%-38%, 15 min) to give the tert-butyl 4-[3-[2-[2-amino-5-methoxy-N-methyl-4-[[4-(1-methylindol-3-yl)pyrimidin-2-yl]amino]anilino]ethylmethyl-amino]propyl]piperazine-1-carboxylate (320 mg, 486 μmol, 65% yield) as a gray solid.

Step 3. A mixture of tert-butyl 4-[3-[2-[2-amino-5-methoxy-N-methyl-4-[[4-(1-methylindol-3-yl)pyrimidin-2-yl]amino]anilino]ethyl-methyl-amino]propyl]piperazine-1-carboxylate (110 mg, 167 μmol, 1.0 equiv) in DCM (3 mL), TFA (1 mL) was degassed and purged with N₂ for 3 times, and then the mixture was stirred at 25° C. for 1 h under N₂ atmosphere. The mixture was concentrated to get the residue. The residue was used for next step without further purification. The 5-methoxy-N1-methyl-N4-[4-(1-methylindol-3-yl)pyrimidin-2-yl]-N1-[2-[methyl(3-piperazin-1-ylpropyl)amino]ethyl]benzene-1,2,4-triamine (90 mg, 161 μmol) was obtained as a yellow oil.

Step 4. A mixture of 5-methoxy-N1-methyl-N4-[4-(1-methylindol-3-yl)pyrimidin-2-yl]-N1-[2-[methyl(3-piperazin-1-ylpropyl)amino]ethyl]benzene-1,2,4-triamine (90 mg, 161. μmol, 1.0 equiv), 3-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]propanal (87 mg, 193 μmol, 1.2 equiv), NaBH(OAc)₃ (171 mg, 806 μmol, 5.0 equiv) in DCM (5 mL) was degassed and purged with N2 for 3 times, and then the mixture was stirred at 25° C. for 1 h under N₂ atmosphere. The mixture was concentrated to give the residue. The residue was purified by the prep-HPLC (column: Welch Xtimate C18 150*25 mm*5 m; mobile phase: [water(TFA)-ACN]; B%: 10%-40%, 10 min) to give the 2-[(2S)-1-[7-[[6-[3-[4-[3-[2-[2-amino-5-methoxy-N-methyl-4-[[4-(1-methylindol-3-yl)pyrimidin-2-yl]amino]anilino]ethyl-methyl-amino]propyl]piperazin-1-yl]propoxy]-3-pyridyl]methylamino]-3-ethyl-pyrazolo[1,5-a]pyrimidin-5-yl]-2-piperidyl]ethanol (35 mg, 35 μmol, 28% yield) as a yellow solid. ¹H NMR (400 MHz, DMSO-d₆) δ =9.42-9.16 (m, 1H), 8.76-8.63 (m, 1H), 8.55 (s, 1H), 8.33-8.15 (m, 3H), 7.91-7.77 (m, 2H), 7.75-7.64 (m, 1H), 7.60-7.52 (m, 1H), 7.34-7.25 (m, 2H), 7.19-7.13 (m, 1H), 7.08 (s, 1H), 6.77 (d, J=8.4 Hz, 1H), 5.74 (s, 1H), 4.67-4.41 (m, 3H), 4.32-4.20 (m, 2H), 4.13 (d, J=12.8 Hz, 1H), 3.96-3.78 (m, 6H), 3.57-3.43 (m, 2H), 3.42 (s, 3H), 3.27-2.91 (m, 10H), 2.84 (s, 4H), 2.77-2.59 (m, 6H), 2.56-2.51 (m, 4H), 2.09-1.84 (m, 5H), 1.75-1.52 (m, 6H), 1.50-1.34 (m, 1H), 1.21-1.10 (m, 3H).

Step 5. A mixture of 2-[(2S)-1-[7-[[6-[3-[4-[3-[2-[2-amino-5-methoxy-N-methyl-4-[[4-(1-methylindol-3-yl)pyrimidin-2-yl]amino]anilino]ethyl-methyl-amino]propyl]piperazin-1-yl]propoxy]-3-pyridyl]methylamino]-3-ethyl-pyrazolo[1,5-a]pyrimidin-5-yl]-2-piperidyl]ethanol (30 mg, 30 μmol, 1.0 equiv), prop-2-enoyl prop-2-enoate (5 mg, 36 μmol, 1.2 equiv), NaHCO₃ (8 M, 6.0 mL) in THF (1 mL) was degassed and purged with N₂ for 3 times, and then the mixture was stirred at 25° C. for 12 h under N₂ atmosphere. The mixture was concentrated to give the residue and was purified by the prep-HPLC(column: Phenomenex Luna C18 150*25 mm*10 m; mobile phase: [water(FA)-ACN]; B%: 14%-44%, 15 min) to give the N-[2-[2-[3-[4-[3-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]propyl]piperazin-1-yl]propyl-methyl-amino]ethyl-methyl-amino]-4-methoxy-5-[[4-(1-methylindol-3-yl)pyrimidin-2-yl]amino]phenyl]prop-2-enamide (5 mg, 5 μmol, 14% yield, 93% purity, FA) as a yellow solid. LC-MS: MS (ES⁺): RT=2.147 min, m/z=1048.9 [M+H⁺]; LCMS Method: 05. ¹H NMR (400 MHz, MeOD) 6=8.78 (s, 1H), 8.48 (d, J=3.2 Hz, 3H), 8.34 (d, J=7.2 Hz, 1H), 8.22-8.05 (m, 3H), 7.78-7.61 (m, 2H), 7.35 (d, J=8.4 Hz, 1H), 7.21-7.03 (m, 3H), 6.95 (s, 1H), 6.65 (d, J=8.4 Hz, 1H), 6.55-6.44 (m, 2H), 5.96-5.76 (m, 1H), 5.54 (s, 1H), 4.55 (s, 2H), 4.02-3.97 (m, 5H), 3.79 (s, 3H), 3.53-3.45 (m, 3H), 3.41-3.34 (m, 2H), 3.23-3.16 (m, 2H), 3.04-2.94 (m, 3H), 2.82 (s, 3H), 2.74 (s, 3H), 2.60-2.49 (m, 3H), 2.32 (s, 8H), 2.13-2.00 (m, 2H), 1.75-1.57 (m, 1OH), 1.25-1.20 (m, 3H).

### Example 134—Synthesis of Compound I-150

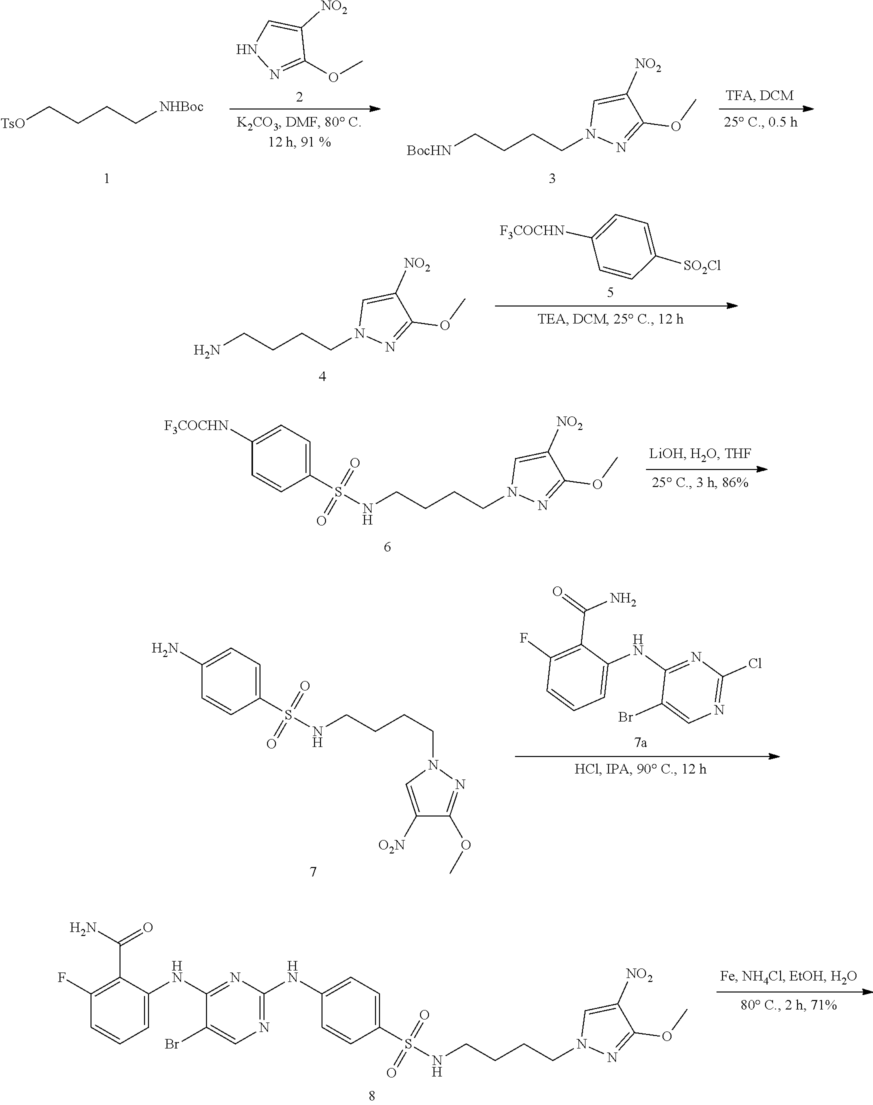

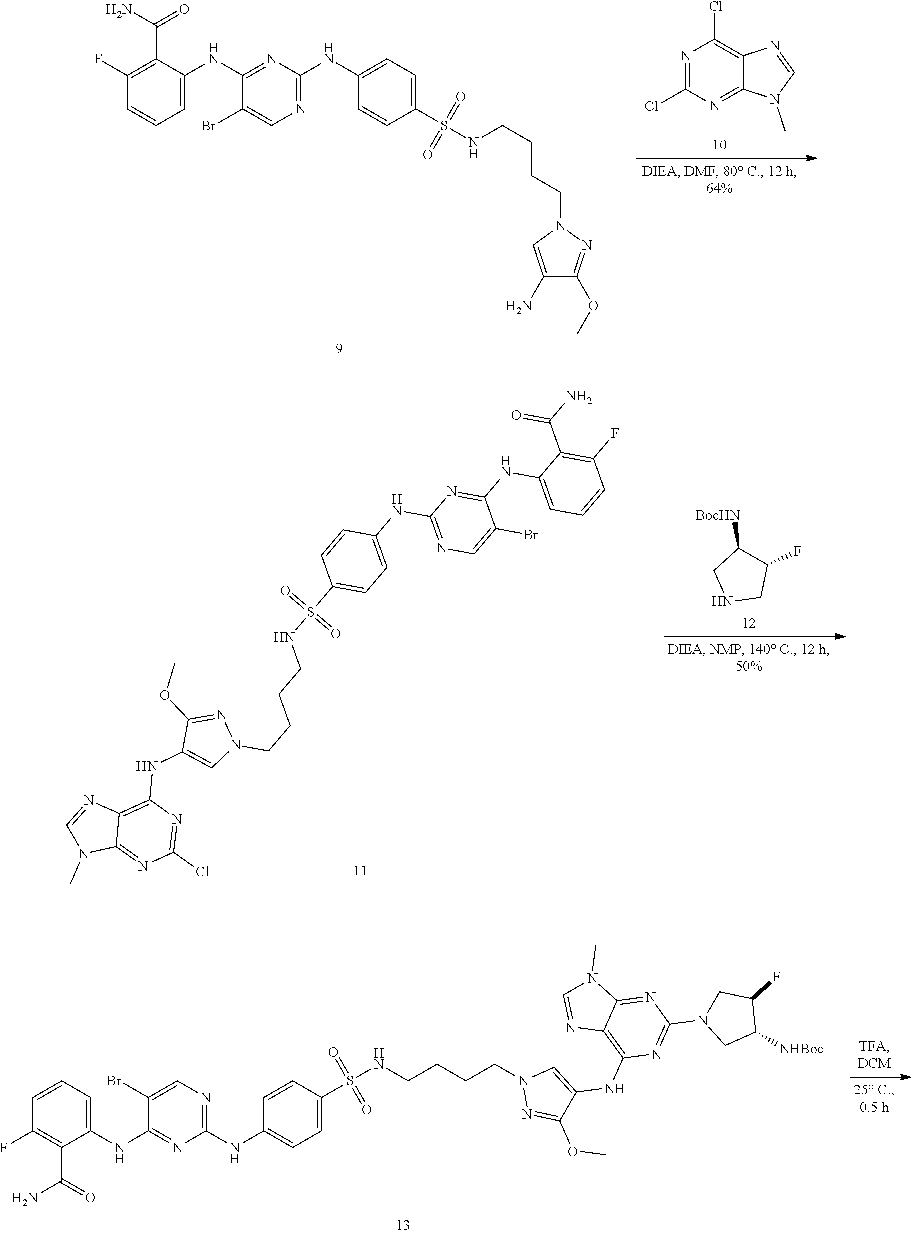 [SMILES: COc1nn(CCCCNS(=O)(=O)c2ccc(Nc3ncc(Br)c(Nc4cccc(F)c4C(N)=O)n3)cc2)cc1N ; Cn1cnc2c(Cl)nc(Cl)nc21 ; COc1nn(CCCCNS(=O)(=O)c2ccc(Nc3ncc(Br)c(Nc4cccc(F)c4C(N)=O)n3)cc2)cc1Nc1nc(Cl)nc2c1ncn2C ; CC(C)(C)OC(=O)N[C@@H]1CNC[C@H]1F ; COc1nn(CCCCNS(=O)(=O)c2ccc(Nc3ncc(Br)c(Nc4cccc(F)c4C(N)=O)n3)cc2)cc1Nc1nc(N2C[C@@H](F)[C@H](NC(=O)OC(C)(C)C)C2)nc2c1ncn2C]

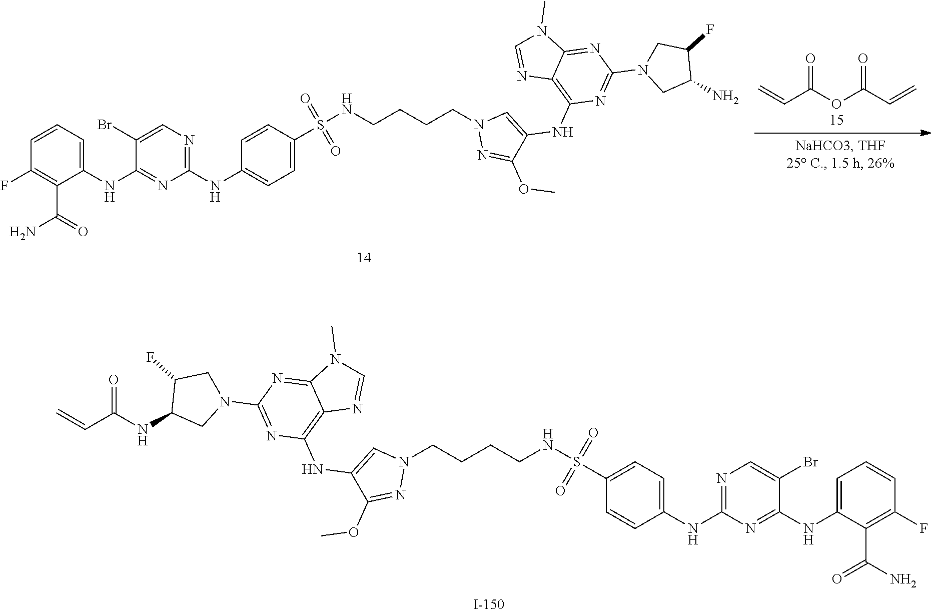

General Information: Compound 7a is a known compound from WO2020/206137, 2020, A1.

Step 1. To a solution of 4-(tert-butoxycarbonylamino) butyl 4-methylbenzenesulfonate (6 g, 17.4 mmol, 1.0 equiv) and 5-methoxy-4-nitro-¹H-pyrazole (2.75 g, 19.2 mmol, 1.1 equiv) in DMF (40 mL) was added K₂CO₃ (7.24 g, 52.41 mmol, 3.0 equiv). The mixture was stirred at 80° C. for 12 h. The reaction mixture was diluted with 500 mL water, and then it was extracted with EtOAc (3×100 mL). The organic layers were dried over anhydrous Na₂SO₄, filtered and concentrated to afford crude product. The residue was purified by column chromatography (SiO₂, Petroleum ether/Ethyl acetate=50/1:10/1) to give a compound tert-butyl N-[4-(3-methoxy-4-nitro-pyrazol-1-yl) butyl]carbamate (5.0 g, 91% yield) as a yellow oil.

Step 2. To a solution of tert-butyl N-[4-(3-methoxy-4-nitro-pyrazol-1-yl) butyl]carbamate (4 g, 12.7 mmol, 1.0 equiv) in DCM (1 mL) was added TFA (0.5 mL). The mixture was stirred at 25° C. for 0.5 h. The reaction mixture was concentrated under reduced pressure to give compound 4-(3-methoxy-4-nitro-pyrazol-1-yl) butan-1-amine (4 g, 96% yield, TFA salt) as a colorless oil.

Step 3. To a solution of 4-(3-methoxy-4-nitro-pyrazol-1-yl)butan-1-amine (4 g, 12.2 mmol, 1.0 equiv, TFA salt) in DCM (40 mL) was added TEA (3.70 g, 36.6 mmol, 5.0 mL, 3.0 equiv) and 4-[(2,2,2-trifluoroacetyl)amino]benzenesulfonyl chloride (4.2 g, 14.6 mmol, 1.2 equiv) at 0° C. The mixture was stirred at 25° C. for 12 h. The residue was diluted with H₂O (60 mL) and extracted with EtOAc (50 mL×3). The combined organic layers were filtered and concentrated under reduced pressure to give 2,2,2-trifluoro-N-[4-[4-(3-methoxy-4-nitro-pyrazol-1-yl)butylsulfamoyl]phenyl]acetamide (5.4 g, 95% yield) as a yellow oil. LC-MS: MS (ES⁺): RT=0.771 min, m/z=466.1 [M+H]+.

Step 4. To a solution of 2,2,2-trifluoro-N-[4-[4-(3-methoxy-4-nitro-pyrazol-1-yl) butylsulfamoyl]phenyl]acetamide (5.0 g, 10.7 mmol, 1.0 equiv) in THF (40 mL) and H₂O (10 mL) was added LiOH.H₂O (901.6 mg, 21.5 mmol, 2.0 equiv). The mixture was stirred at 25° C. for 3 h. The residue was diluted with H₂O (40 mL) and extracted with EtOAc (50 mL×3). The combined organic layers were filtered and concentrated under reduced pressure to give a compound 4-amino-N-[4-(3-methoxy-4-nitro-pyrazol-1-yl)butyl]benzenesulfonamide (3.4 g, 86% yield) as a yellow oil. ¹H NMR (400 MHz, CDCl₃): δ7.94 (s, 1H), 7.53-7.69 (m, 2H), 6.58-6.75 (m, 2H), 4.03 (s, 3H), 3.72-3.77 (m, 2H), 2.96 (s, 2H), 1.83-1.91 (m, 4H).

Step 5. To a solution of 2-[(5-bromo-2-chloro-pyrimidin-4-yl)amino]-6-fluoro-benzamide (2.2 g, 6.4 mmol, 1.0 equiv) and 4-amino-N-[4-(3-methoxy-4-nitro-pyrazol-1-yl) butyl]benzenesulfonamide (2.3 g, 6.3 mmol, 1.0 equiv) in IPA (20 mL) was added HCl (12 M, 530.5 μL, 1.0 equiv). The mixture was stirred at 90° C. for 12 h. The reaction mixture was filtered and concentrated under reduced pressure to give a compound 2-[[5-bromo-2-[4-[4-(3-methoxy-4-nitro-pyrazol-1-yl)butylsulfamoyl]anilino]pyrimidin-4-yl]amino]-6-fluoro-benzamide (4.2 g, 97% yield) as a yellow solid. ¹H NMR (400 MHz, DMSO): δ 10.03-10.26 (m, 2H), 8.65-8.77 (m, 1H), 8.41 (s, 1H), 8.09-8.26 (m, 3H), 7.82 (d, J=8.80 Hz, 2H), 7.62 (d, J=8.80 Hz, 2H), 7.42-7.59 (m, 2H), 7.08-7.13 (m, 1H), 6.78 (d, J=8.68 Hz, 1H), 3.95-3.99 (m, 2H), 3.88-3.93 (m, 4H), 2.69-2.77 (m, 2H), 1.70-1.78 (m, 2H), 1.31-1.46 (m, 2H).

Step 6. To a solution of 2-[[5-bromo-2-[4-[4-(3-methoxy-4-nitro-pyrazol-1-yl)butylsulfamoyl]anilino]pyrimidin-4-yl]amino]-6-fluoro-benzamide (2.2 g, 3.24 mmol, 1.0 equiv) in EtOH (300 mL) and H₂O (30 mL) was added Fe (1.81 g, 32.42 mmol, 10.0 equiv) and NH₄Cl (1.73 g, 32.4 mmol, 10.0 equiv). The mixture was stirred at 80° C. for 2 h. The reaction mixture was concentrated under reduced pressure to remove solvent. The residue was diluted with H₂O (100 mL) and extracted with EA (150 mL×3). The combined organic layers were filtered and concentrated under reduced pressure to give 2-[[2-[4-[4-(4-amino-3-methoxy-pyrazol-1-yl) butylsulfamoyl]anilino]-5-bromo-pyrimidin-4-yl]amino]-6-fluoro-benzamide (1.5 g, 71% yield) as a white solid.

Step 7. To a solution of 2-[[2-[4-[4-(4-amino-3-methoxy-pyrazol-1-yl)butyl sulfamoyl]anilino]-5-bromo-pyrimidin-4-yl]amino]-6-fluoro-benzamide (1.5 g, 2.3 mmol, 1.0 equiv), 2,6-dichloro-9-methyl-purine (469.6 mg, 2.3 mmol, 1.0 equiv) in DMF (20 mL) was added DIEA (597.8 mg, 4.6 mmol, 805.7 μL, 2.0 equiv). The mixture was stirred at 80° C. for 12 h. The residue was diluted with H₂O (100 mL) and extracted with EA (100 mL×3). The combined organic layers were filtered and concentrated under reduced pressure to give a residue. The residue was purified by column chromatography (SiO₂, Petroleum ether/Ethyl acetate=10/1 to 2/1) to give 2-[[5-bromo-2-[4-[4-[4-[(2-chloro-9-methyl-purin-6-yl)amino]-3-methoxy-pyrazol-1-yl]butylsulfamoyl]anilino]pyrimidin-4-yl]amino]-6-fluoro-benzamide (1.2 g, 64% yield) as a white solid.

Step 8. To a solution of 2-[[5-bromo-2-[4-[4-[4-[(2-chloro-9-methyl-purin-6-yl)amino]-3-methoxy-pyrazol-1-yl]butylsulfamoyl]anilino]pyrimidin-4-yl]amino]-6-fluoro-benzamide (240 mg, 294.5 μmol, 1.0 equiv) and tert-butyl N-[(3R)-4-fluoropyrrolidin-3-yl]carbamate (120.3 mg, 588.9 μmol, 2.0 equiv) in NMP (1 mL) was added DIEA (114.2 mg, 883.4 μmol, 153.8 μL, 3.0 equiv). The mixture was stirred at 140° C. for 12 h. The residue was purified by prep-HPLC (column: Phenomenex C18 75*30 mm*3 m; mobile phase: [water (FA)-ACN]; B%: 38%-68%, 7 min) to afford tert-butyl N-[(3R)-1-[6-[[1-[4-[[4-[[5-bromo-4-(2-carbamoyl-3-fluoro-anilino)pyrimidin-2-yl]amino]phenyl]sulfonylamino]butyl]-3-methoxy-pyrazol-4-yl]amino]-9-methyl-purin-2-yl]-4-fluoro-pyrrolidin-3-yl]carbamate (145 mg, 50% yield) as a gray solid.

Step 9. To a solution of tert-butyl N-[(3R)-1-[6-[[1-[4-[[4-[[5-bromo-4-(2-carbamoyl-3-fluoro-anilino)pyrimidin-2-yl]amino]phenyl]sulfonylamino]butyl]-3-methoxy-pyrazol-4-yl]amino]-9-methyl-purin-2-yl]-4-fluoro-pyrrolidin-3-yl]carbamate (80 mg, 81.4 μmol, 1.0 equiv) in DCM (0.5 mL) and TFA (0.2 mL), and then it was stirred at 25° C. for 0.5 h. The reaction mixture was concentrated to afford 2-[[2-[4-[4-[4-[[2-[(3R)-3-amino-4-fluoro-pyrrolidin-1-yl]-9-methyl-purin-6-yl]amino]-3-methoxy-pyrazol-1-yl]butylsulfamoyl]anilino]-5-bromo-pyrimidin-4-yl]amino]-6-fluoro-benzamide (81 mg, 99% yield, TFA salt) as a colorless oil.

Step 10. To a solution of 2-[[2-[4-[4-[4-[[2-[(3R)-3-amino-4-fluoro-pyrrolidin-1-yl]-9-methyl-purin-6-yl]amino]-3-methoxy-pyrazol-1-yl]butylsulfamoyl]anilino]-5-bromo-pyrimidin-4-yl]amino]-6-fluoro-benzamide (64 mg, 64.2 μmol, 1.0 equiv, TFA salt) in THF (0.5 mL) was added NaHCO₃ (205 mg, 2.43 mmol) in H₂O (0.5 mL) and prop-2-enoyl prop-2-enoate (8.9 mg, 70.6 μmol, 1.1 equiv). The mixture was stirred at 0° C. for 1.5 h. The reaction mixture was concentrated to afford crude product. The residue was purified by prep-HPLC (column: Waters Xbridge 150*25 mm*5 m; mobile phase: [water(NH4HCO₃)-ACN]; B%: 28%-58%, 9 min) to give compound 2-[[5-bromo-2-[4-[4-[4-[[2-[(4R)-3-fluoro-4-(prop-2-enoylamino)pyrrolidin-1-yl]-9-methyl-purin-6-yl]amino]-3-methoxy-pyrazol-1-yl]butylsulfamoyl]anilino]pyrimidin-4-yl]amino]-6-fluoro-benzamide (17 mg, 26% yield) as a white solid. ¹H NMR (400 MHz, DMSO-d₆): δ10.10 (s, 1H), 9.84-9.94 (m, 1H), 8.42-8.51 (m, 1H), 8.37 (s, 1H), 8.07-8.28 (m, 3H), 7.99 (s, 1H), 7.75-7.92 (m, 3H), 7.59-7.69 (m, 2H), 7.46-7.56 (m, 1H), 7.40-7.43 (m, 1H), 7.06-7.10 (m, 1H), 6.06-6.26 (m, 2H), 5.56-5.66 (m, 1H), 5.16-5.27 (m, 1H), 5.03-5.10 (m, 1H), 4.40-4.49 (m, 1H), 3.57-3.93 (m, 10H), 2.67 (d, J=1.71 Hz, 2H), 2.27-2.37 (m, 1H), 1.65-1.76 (m, 2H), 1.32-1.42 (m, 2H). LC-MS: MS (ES′): RT=2.27 min, m/z=938.0 [M+H⁺]; LCMS method: 25.

### Example 135—Synthesis of Compound I-151

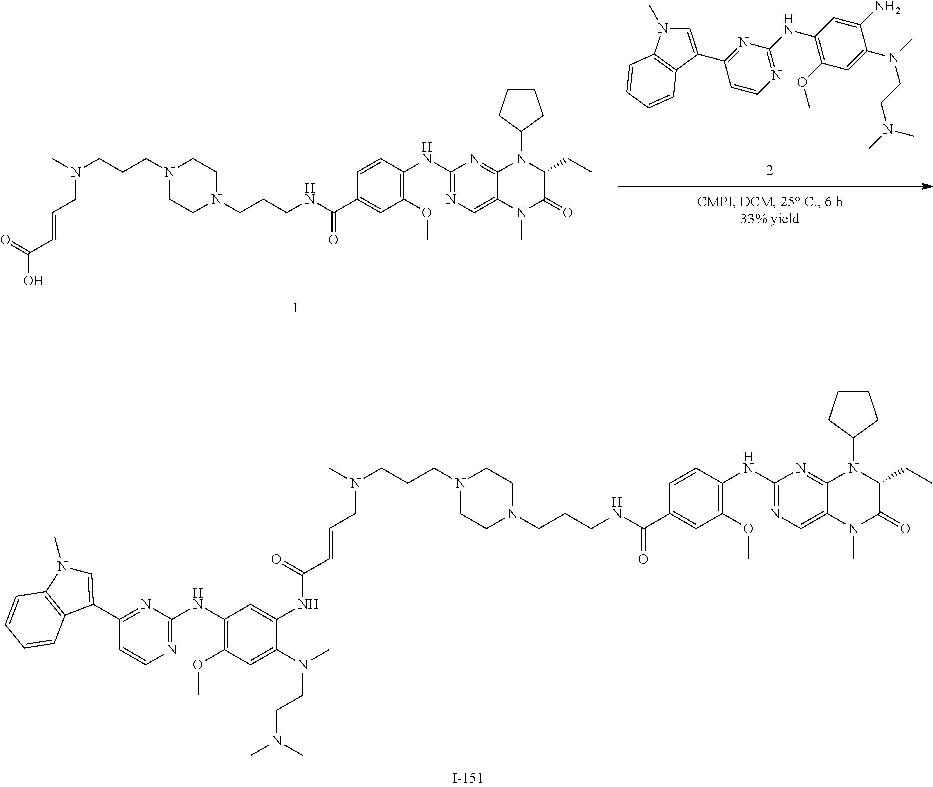 [SMILES: CC[C@@H]1C(=O)N(C)c2cnc(Nc3ccc(C(=O)NCCCN4CCN(CCCN(C)C/C=C/C(=O)O)CC4)cc3OC)nc2N1C1CCCC1 ; COc1cc(N(C)CCN(C)C)c(N)cc1Nc1nccc(-c2cn(C)c3ccccc23)n1 ; CC[C@@H]1C(=O)N(C)c2cnc(Nc3ccc(C(=O)NCCCN4CCN(CCCN(C)C/C=C/C(=O)Nc5cc(Nc6nccc(-c7cn(C)c8ccccc78)n6)c(OC)cc5N(C)CCN(C)C)CC4)cc3OC)nc2N1C1CCCC1]

General Information: Synthetic route of compound 2 is reported in CN107216313.

A mixture of (E)-4-[3-[4-[3-[[4-[[(7R)-8-cyclopentyl-7-ethyl-5-methyl-6-oxo-7H-pteridin-2-yl]amino]-3-methoxy-benzoyl]amino]propyl]piperazin-1-yl]propyl-methyl-amino]but-2-enoic acid (150 mg, 212 μmol, 1.0 equiv), N1-[2-(dimethylamino)ethyl]-5-methoxy-N1-methyl-N4-[4-(1-methylindol-3-yl)pyrimidin-2-yl]benzene-1,2,4-triamine (113 mg, 255 μmol, 1.2 equiv), CMPI (65 mg, 255 μmol, 1.2 equiv), TEA (64 mg, 637 μmol, 88 μL, 3.0 equiv) in DCM (3 mL) was degassed and purged with N₂ for 3 times, and then the mixture was stirred at 25° C. for 6 h under N₂ atmosphere. The mixture was purified by the column: YMC-Actus Triart C18 150*30 mm*7 m; mobile phase: [water (ammonia hydroxide v/v)-MeOH]; B%: 0%-0%, 11 min) to give the 4-[[(7R)-8-cyclopentyl-7-ethyl-5-methyl-6-oxo-7H-pteridin-2-yl]amino]-N-[3-[4-[3-[[(E)-4-[2-[2-(dimethylamino)ethyl-methyl-amino]-4-methoxy-5-[[4-(1-methylindol-3-yl)pyrimidin-2-yl]amino]anilino]-4-oxo-but-2-enyl]-methyl-amino]propyl]piperazin-1-yl]propyl]-3-methoxy-benzamide (7 mg, 6 μmol, 8% yield) as a yellow oil. LC-MS: MS (ES′): RT=1.985 min, m/z=1134.0 [M+H⁺]; LCMS Method: 10. ¹H NMR: (400 MHz, MeOD) δ=8.59 (s, 1H), 8.45-8.17 (m, 1H), 8.01 (d, J=6.0 Hz, 1H), 7.93-7.86 (m, 1H), 7.68-7.58 (m, 2H), 7.54 (d, J=8.2 Hz, 1H), 7.41 (d, J=7.2 Hz, 1H), 7.38-7.32 (m, 1H), 7.31-7.25 (m, 1H), 7.19 (d, J=15.2 Hz, 1H), 7.09 (s, 1H), 7.01-6.91 (m, 1H), 4.51-4.48 (m, 1H), 4.43-4.17 (m, 3H), 4.11 (d, J=5.2 Hz, 2H), 4.03-3.70 (m, 13H), 3.65-3.51 (m, 4H), 3.49-3.32 (m, 7H), 3.07-2.87 (m, 9H), 2.86-2.68 (m, 4H), 2.58-2.48 (m, 1H), 2.43-2.29 (m, 2H), 2.22-2.12 (m, 2H), 2.11-1.99 (m, 3H), 1.97-1.78 (m, 4H), 1.76-1.49 (m, 5H), 0.89-0.80 (m, 3H).

### Example 136—Synthesis of Compound I-152

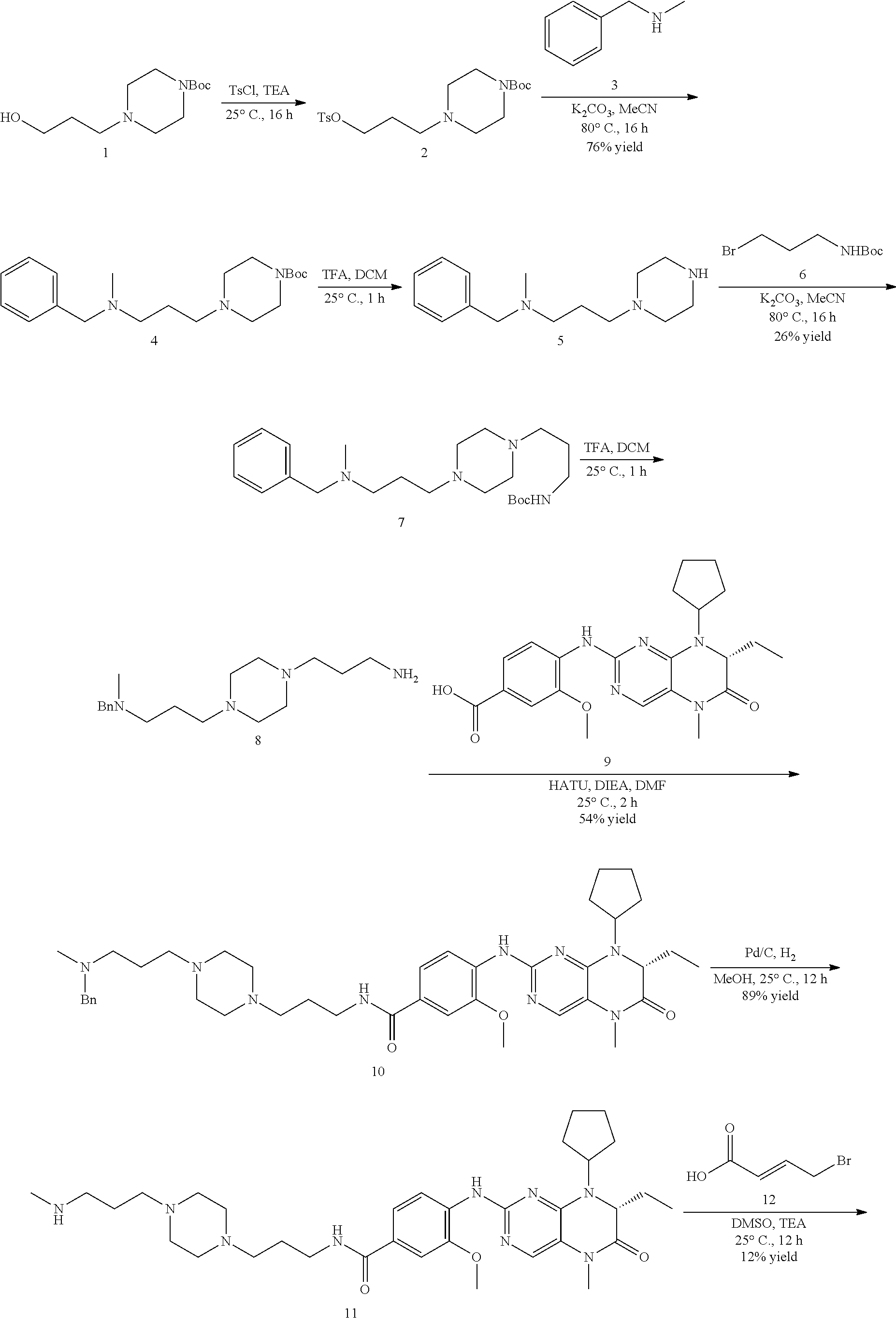

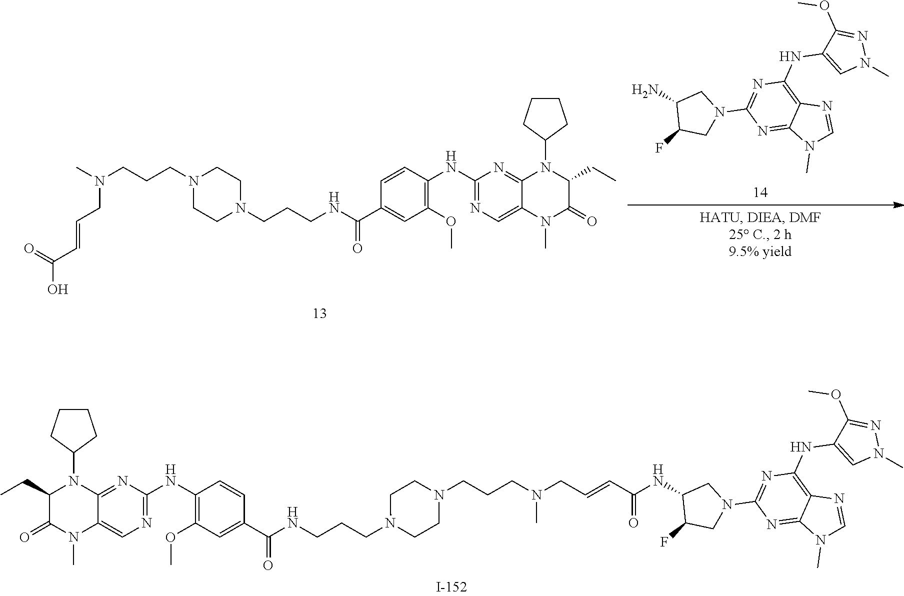

General Information: The compound 9 is reported in: WO2017/24317, 2017, A2.

Step 1. To a solution of tert-butyl 4-(3-hydroxypropyl)piperazine-1-carboxylate (10.0 g, 40.9 mmol, 1.0 equiv) in DCM (120 mL) was added TosCl (11.7 g, 61.4 mmol, 1.5 equiv) and TEA (8.3 g, 81.9 mmol, 11.4 mL, 2.0 equiv). The mixture was stirred at 25° C. for 16 h. The mixture was quenched by NaHCO₃ (100 ml), extracted with DCM (100 mL×2), the combined organic was washed with brine (100 mL×2), dried over Na₂SO₄, and the filtrate was concentrated to give a crude tert-butyl 4-[3-(p-tolylsulfonyloxy)propyl]piperazine-1-carboxylate (12.3 g, crude) as a brown oil, it was used to the next step without further purification.

Step 2. To a solution of tert-butyl 4-[3-(p-tolylsulfonyloxy)propyl]piperazine-1-carboxylate (12.3 g, 30.8 mmol, 1.0 equiv) and N-methyl-1-phenyl-methanamine (3.7 g, 30.8 mmol, 4.0 mL, 1.0 equiv) in MeCN (120 mL) was added K₂CO₃ (8.5 g, 61.7 mmol, 2.0 equiv) at 25° C. The mixture was stirred at 80° C. for 16 h. The mixture was filtered, the filtrate was concentrated to give a residue. The residue was purified by flash silica gel chromatography (eluent of 0˜10% MeOH/DCM) to give tert-butyl 4-[3-[benzyl(methyl)amino]propyl]piperazine-1-carboxylate (8.5 g, 24.0 mmol, 76% yield) as a light yellow oil. ¹H NMR: (400 MHz, CDCl₃) δ7.32-7.29 (m, 4H), 7.23 (s, 1H), 3.49 (s, 2H), 3.44-3.40 (m, 4H), 2.41-2.35 (m, 8H), 2.20 (s, 3H), 1.71 (t, J=7.2 Hz, 2H), 1.46 (s, 9H).

Step 3. To a solution of tert-butyl 4-[3-[benzyl(methyl)amino]propyl]piperazine-1-carboxylate (8.5 g, 24.4 mmol, 1.0 equiv) in DCM (50.0 mL) was added TFA (15.4 g, 135 mmol, 10.0 mL, 5.5 equiv) at 25° C. The mixture was stirred at 25° C. for 1 h. The mixture was concentrated to give a crude N-benzyl-N-methyl-3-piperazin-1-yl-propan-1-amine (12.0 g, crude, TFA salt) as a light yellow oil.

Step 4. To a solution of N-benzyl-N-methyl-3-piperazin-1-yl-propan-1-amine (12.0 g, 48.5 mmol, 1.0 equiv) in MeCN (150 mL) was added K₂CO₃ (20.1 g, 146 mmol, 3.0 equiv) and tert-butyl N-(3-bromopropyl)carbamate (11.6 g, 48.5 mmol, 1.0 equiv) at 25° C. The mixture was stirred at 80° C. for 16 h. The mixture was cooled to 25° C., filtered, the filtrate was concentrated to give a residue. The residue was purified by flash silica gel chromatography (eluent of 0˜10% MeOH/DCM) to give tert-butyl N-[3-[4-[3-[benzyl(methyl)amino]propyl]piperazin-1-yl]propyl]carbamate (5.1 g, 12.5 mmol, 26% yield,) as a light yellow oil. ¹H NMR: (400 MHz, CDCl₃) δ7.33-7.27 (m, 4H), 7.26-7.20 (m, 1H), 3.48 (s, 2H), 3.37 (t, J=2.0, 6.0 Hz, 1H), 3.18 (d, J=5.6 Hz, 2H), 2.47 (d, J=5.2 Hz, 4H), 2.44-2.33 (m, 8H), 2.19 (s, 3H), 2.03-1.93 (m, 1H), 1.76-1.69 (m, 2H), 1.67-1.60 (m, 2H), 1.43 (s, 9H).

Step 5. A mixture of tert-butyl N-[3-[4-[3-[benzyl(methyl)amino]propyl]piperazin-1-yl]propyl]carbamate (1.5 g, 3.7 mmol, 1.0 equiv) in DCM (15 mL), TFA (5 mL) was degassed and purged with N2 for 3 times, and then the mixture was stirred at 25° C. for 1 h under N2 atmosphere. The mixture was concentrated to give the residue. Compound 3-[4-[3-[benzyl(methyl) amino]propyl]piperazin-1-yl]propan-1-amine (1.6 g, 3.7 mmol, TFA) was obtained as a yellow oil.

Step 6. A mixture of 4-[[(7R)-8-cyclopentyl-7-ethyl-5-methyl-6-oxo-7H-pteridin-2-yl]amino]-3-methoxy-benzoic acid (1.2 μg, 2.8 μmmol, 1.0 equiv), 3-[4-[3-[benzyl (methyl)amino]propyl]piperazin-1-yl]propan-1-amine (1.4 g, 3.4 mmol, 1.2 equiv), HATU (1.1 g, 2.8 mmol, 1.1 equiv), DIEA (1.1 g, 8.5 mmol, 1.5 mL, 3.0 equiv) in DMF (15 mL) was degassed and purged with N₂ for 3 times, and then the mixture was stirred at 25° C. for 2 h under N₂ atmosphere. The mixture was concentrated to give the residue and was purified by the prep-HPLC (column: Phenomenex Luna C18 200*40 mm*10 m; mobile phase: [water(HCl)-ACN]; B%: 3%-33%, 10 min) to give the N-[3-[4-[3-[benzyl(methyl)amino]propyl]piperazin-1-yl]propyl]-4-[[(7R)-8-cyclopentyl-7-ethyl-5-methyl-6-oxo-7H-pteridin-2-yl]amino]-3-methoxy-benzamide (1.1 g, 1.6 mmol, 54% yield) as a yellow solid. ¹H NMR: (400 MHz, MeOD) 6=7.91 (d, J=8.4 Hz, 1H), 7.69-7.62 (m, 2H), 7.62-7.56 (m, 3H), 7.55-7.48 (m, 3H), 4.58-4.45 (m, 2H), 4.41-4.27 (m, 2H), 4.01 (s, 6H), 3.85-3.72 (m, 3H), 3.61-3.54 (m, 2H), 3.47-3.37 (m, 6H), 3.33-3.31 (m, 2H), 3.02 (s, 1H), 2.85 (s, 3H), 2.50-2.32 (m, 2H), 2.25-1.83 (m, 9H), 1.77-1.52 (m, 4H), 1.44-1.28 (m, 1H), 0.95-0.73 (m, 3H).

Step 7. A mixture of N-[3-[4-[3-[benzyl(methyl)amino]propyl]piperazin-1-yl]propyl]-4-[[(7R)-8-cyclopentyl-7-ethyl-5-methyl-6-oxo-7H-pteridin-2-yl]amino]-3-methoxy-benzamide (1.0 g, 1.4 mmol, 1.0 equiv), Pd/C (200 mg, 10% purity) in MeOH (10 mL) was degassed and purged with H₂ for 3 times, and then the mixture was stirred at 25° C. for 12 h under H₂ atmosphere. The mixture was filtered and concentrated to give 4-[[(7R)-8-cyclopentyl-7-ethyl-5-methyl-6-oxo-7H-pteridin-2-yl]amino]-3-methoxy-N-[3-[4-[3-(methylamino)propyl]piperazin-1-yl]propyl]benzamide (780 mg, 1.3 mmol, 89% yield) as a colorless oil.

Step 8. A mixture of 4-[[(7R)-8-cyclopentyl-7-ethyl-5-methyl-6-oxo-7H-pteridin-2-yl]amino]-3-methoxy-N-[3-[4-[3-(methylamino)propyl]piperazin-1-yl]propyl]benzamide (700 mg, 1.1 mmol, 1.0 equiv), (E)-4-bromobut-2-enoic acid (185 mg, 1.1 mmol, 1.0 equiv), TEA (227 mg, 2.3 mmol, 313 μL, 2.0 equiv) in DMSO (8 mL) was degassed and purged with N₂ for 3 times, and then the mixture was stirred at 25° C. for 12 h under N₂ atmosphere. The mixture was filtered and concentrated to give the residue and was purified by the prep-HPLC (column: Phenomenex luna C18 250*50 mm*10 m; mobile phase: [water(NH₄HCO₃)-ACN]; B%: 20%-50%, 10 min) to give the (E)-4-[3-[4-[3-[[4-[[(7R)-8-cyclopentyl-7-ethyl-5-methyl-6-oxo-7H-pteridin-2-yl]amino]-3-methoxy-benzoyl]amino]propyl]piperazin-1-yl]propyl-methyl-amino]but-2-enoic acid (100 mg, 141 μmol, 12% yield) as a white solid. ¹H NMR: (400 MHz,DMSO-d₆) δ =8.41 (d, J=8.4 Hz, 1H), 8.38-8.33 (m, 1H), 7.84 (s, 1H), 7.60 (s, 1H), 7.52-7.37 (m, 2H), 6.75-6.62 (m, 1H), 5.88 (d, J=15.6 Hz, 1H), 4.39-4.32 (m, 1H), 4.27-4.21 (m, 1H), 3.93 (s, 3H), 3.29-3.22 (m, 6H), 3.06 (d, J=5.6 Hz, 2H), 2.41-2.20 (m, 13H), 2.12 (s, 3H), 2.06-1.97 (m, 1H), 1.93-1.51 (m, 14H), 0.83-0.68 (m, 3H).

Step 9. A mixture of (E)-4-[3-[4-[3-[[4-[[(7R)-8-cyclopentyl-7-ethyl-5-methyl-6-oxo-7H-pteridin-2-yl]amino]-3-methoxy-benzoyl]amino]propyl]piperazin-1-yl]propyl-methyl-amino]but-2-enoic acid (40 mg, 57 μmol, 1.0 equiv), 2-[(3R,4R)-3-amino-4-fluoro-pyrrolidin-1-yl]-N-(3-methoxy-1-methyl-pyrazol-4-yl)-9-methyl-purin-6-amine (25 mg, 68 μmol, 1.2 equiv), HATU (26 mg, 68 μmol, 1.2 equiv), TEA (17 mg, 170 μmol, 24 μL, 3.0 equiv) in DMF (2 mL) was degassed and purged with N₂ for 3 times, and then the mixture was stirred at 25° C. for 2 h under N₂ atmosphere. The mixture was purified by the prep-HPLC(column: Phenomenex luna C18 150*25 mm*10 m; mobile phase: [water(FA)-ACN]; B%: 3%-33%, 15 min) to give the 4-[[(7R)-8-cyclopentyl-7-ethyl-5-methyl-6-oxo-7H-pteridin-2-yl]amino]-N-[3-[4-[3-[[(E)-4-[[(3R,4R)-4-fluoro-1-1[6-1[(3-methoxy-1-methyl-pyrazol-4-yl)amino]-9-methyl-purin-2-yl]pyrrolidin-3-yl]amino]-4-oxo-but-2-enyl]-methyl-amino]propyl]piperazin-1-yl]propyl]-3-methoxy-benzamide (6 mg, 5.4 μmol, 9.5% yield, 95% purity) as a white solid. LC-MS: MS (ES⁺): RT=1.983 min, m/z=1049.9 [M+H⁺]; LCMS Method: 05. ¹H NMR: (400 MHz, MeOD) δ=8.52-8.45 (m, 1H), 8.41 (s, 1H), 7.94 (s, 1H), 7.77-7.66 (m, 2H), 7.53-7.42 (m, 2H), 6.91-6.75 (m, 1H), 6.21 (d, J=15.2 Hz, 1H), 5.29-5.07 (m, 1H), 4.65-4.42 (m, 2H), 4.30-4.20 (m, 1H), 4.08-3.88 (m, 9H), 3.80-3.66 (m, 7H), 3.52-3.40 (m, 4H), 3.01-2.77 (m, 10H), 2.70 (d, J=5.5 Hz, 4H), 2.45 (s, 3H), 2.22-2.12 (m, 1H), 2.02-1.68 (m, 14H), 0.89-0.77 (m, 3H).

### Example 137—Cellular Growth Inhibition Assay for HEK293 Cells and HeLa Cells

Exemplary compounds were tested for ability to inhibit the proliferation of HEK293 cells or HeLa cells. Experimental procedures and results are provided below.

### Part I—Experimental Procedure

HEK293 and HeLa cells were cultured in DMEM medium supplemented with 10% fetal bovine serum and 1% Penn/Strep. Cells were seeded in white 384-well plates at 500 cells/well in 25 mL complete medium. Following seeding, plates were spun at 300×g for three minutes and cultured at 37° C. with 5% CO₂ in a humidified tissue culture incubator.

After 24 hours, compounds were titrated in 100% DMSO and diluted in complete cell culture medium. 25 mL of compound/media mixture was added to cells to bring total volume in well to 50 mL. DMSO alone was used as a negative control. P lates were then spun at 300×g for three minutes and stored at 37° C. with 5% CO₂ for three days.

On Day 0 and Day 3 of compound treatment, cell viability was quantified with CellTiter-Glo 2.0 reagent (Promega). After equilibrating microplates at room temperature for 30 minutes, 25 μL CellTiter-Glo 2.0 reagent was dispensed into each well to bring total volume to 75 mL. Plates were mixed on shaker for 2 minutes at 500 rpm, followed by a 10-minute incubation at room temperature. Following a quick spin, luminescence readings were measured with an EnVision Plate Reader. Data was normalized to DMSO treated Day 0 and Day 3 readings. A four-parameter non-linear regression curve fit was applied to dose-response data in GraphPad Prism data analysis software to determine the half maximal growth inhibitory concentration (GI₅₀) for each compound.

### Part II—Results

Results are provided in Table 3 below for exemplary compounds. The symbol “++++” indicates a GI₅₀ less than 0.5 mM. The symbol “+++” indicates an GI₅₀ in the range of 0.5 mM to 1.5 mM. The symbol “++” indicates a GI₅₀ in the range of greater than 1.5 mM to 5 mM. The symbol “+” indicates a GI₅₀ greater than 5 mM. The symbol “N/A” indicates that no data was available.

TABLE 3

| Compound | CTG HEK293 | CTG HELA |
|---|---|---|
| No. | GI₅₀ | GI₅₀ |
| I-67 | +++ | +++ |
| I-69 | +++ | ++++ |
| I-76 | + | + |
| I-78 | +++ | ++++ |
| I-80 | +++ | ++++ |
| I-82 | ++++ | ++++ |
| I-83 | ++ | + |
| I-97 | N/A | +++ |
| I-113 | ++ | N/A |
| I-114 | ++ | N/A |
| I-115 | +++ | N/A |
| I-116 | ++ | N/A |
| I-117 | +++ | N/A |
| I-118 | +++ | N/A |
| I-119 | ++ | N/A |
| I-120 | +++ | N/A |
| I-121 | +++ | N/A |
| I-102 | +++ | N/A |
| I-103 | +++ | N/A |
| I-104 | +++ | N/A |
| I-105 | +++ | N/A |
| I-122 | ++ | ++ |
| I-123 | +++ | +++ |
| I-86 | +++ | ++++ |
| I-87 | +++ | +++ |
| I-88 | +++ | +++ |
| I-98 | ++ | +++ |
| I-124 | ++++ | ++++ |
| I-125 | ++ | + |
| I-126 | ++++ | +++ |
| I-127 | +++ | ++ |
| I-89 | +++ | ++++ |
| I-106 | +++ | +++ |
| I-99 | ++ | +++ |
| I-128 | +++ | +++ |
| I-129 | +++ | +++ |
| I-130 | +++ | +++ |
| I-131 | +++ | +++ |
| I-107 | ++++ | ++++ |
| I-108 | ++ | +++ |
| I-109 | ++ | +++ |
| I-132 | ++++ | ++++ |
| I-133 | ++++ | ++++ |
| I-110 | +++ | +++ |
| I-100 | + | ++ |
| I-101 | ++ | ++ |
| I-134 | + | ++ |
| I-135 | + | + |
| I-90 | ++++ | ++++ |
| I-136 | +++ | ++++ |
| I-137 | + | ++ |
| I-138 | + | + |
| I-139 | ++++ | ++++ |
| I-140 | ++++ | ++++ |
| I-141 | ++++ | ++++ |
| I-91 | ++++ | ++++ |
| I-142 | + | ++ |
| I-143 | + | + |
| I-144 | ++++ | ++++ |
| I-145 | ++++ | ++++ |
| I-92 | +++ | ++++ |
| I-93 | ++ | ++++ |
| I-146 | | +++ |
| I-147 | ++++ | +++ |
| I-94 | ++ | +++ |
| I-95 | ++ | + |
| I-148 | ++++ | ++++ |
| I-149 | ++++ | ++++ |
| I-150 | +++ | + |
| I-151 | +++ | ++++ |
| I-152 | + | ++++ |

### Example 138—Assay for Inhibition of KRAS G12C

Exemplary compounds were tested for ability to inhibit KRas G12C. Experimental procedures and results are provided below.

### Part I—Experimental Procedure

KRAS target engagement (IC₅₀ determination) was performed using a KRAS G12C nucleotide exchange assay (Reaction Biology Corporation). Specifically, compounds were tested against KRAS G12C in 10-point concentration IC₅₀ mode with 3-fold serial dilution at a starting concentration of 10 μM. ARS1620 was used as a control. Compounds were pre-incubated with protein for 1 hr at RT. Parameters for the assay were:

| Protein: | Kras (aa 2-169), GST | RBC | |
|---|---|---|---|
| | SOS1 (aa 564-1049) | RBC | |
| Substrate: | Kras G12C | | |
| | Kras | 30 | nM |
| | SOS1 | 20 | nM |
| | GTP-DY-647P1 | 0.15 | mM |

| | |
|---|---|
| Detection: | SOS1 mediated exchange |
| | of GDP to GTP-DY-647P1 |
| | HTRF measurement |
| | (Ex/Em = (320-75/665-7.5; |
| | 615-8.5)) in Envision (Perkin Elmer) |

In the method, Kras was mixed with a-GST Tb antibody (1.5× solution) and 10 mL was delivered to reaction wells. Compounds were delivered using acoustic dispenser (Echo, Labcyte). Kras/aGST-Tb Ab/compound mixture was incubated for 1 hour at room temperature. A 3× solution of SOS1 and GTP* was prepared in reaction buffer. A 5 mL aliquot of SOS1/GTP* solution was added to reaction well to initiate the exchange reaction. No—SOS1 reaction or max compound concentration was used as blank. IC₅₀ determination was performed using a Sigmoidal dose response (variable slope) equation.

### Part II—Results

Results showing inhibition of KRas G12C are provided in Table 4 below. The symbol “++++” indicates a IC₅₀ less than 0.1 mM. The symbol “+++” indicates an IC₅₀ in the range of 0.1 mM to 1 mM. The symbol “++” indicates a IC₅₀ in the range of greater than 1 mM to 5 mM. The symbol “+” indicates a IC₅₀ greater than 5 mM. The symbol “N/A” indicates that no data was available.

TABLE 4

| Compound No. | IC₅₀ |
|---|---|
| I-21 | +++ |
| I-63 | ++++ |
| I-65 | +++ |
| I-77 | ++ |
| I-78 | ++++ |
| I-79 | ++ |
| I-80 | ++++ |
| I-82 | ++++ |
| I-96 | ++++ |
| I-97 | ++++ |
| I-102 | +++ |
| I-103 | ++++ |
| I-104 | ++++ |
| I-105 | ++++ |
| I-86 | +++ |
| I-87 | ++++ |
| I-88 | ++++ |
| I-98 | ++++ |
| I-89 | ++++ |
| I-106 | ++++ |
| I-99 | ++++ |
| I-107 | ++++ |
| I-108 | ++++ |
| I-109 | ++++ |
| I-110 | ++++ |
| I-100 | ++++ |
| I-101 | ++++ |
| I-90 | ++++ |
| I-91 | ++++ |
| I-92 | ++++ |
| I-93 | ++++ |
| I-94 | ++++ |
| I-95 | ++ |

### Example 139—Assay for Binding Affinity to EGFR

Exemplary compounds were tested for ability to bind to EGFR. Experimental procedures and results are provided below.

### Part I—Experimental Procedure

Compounds were tested using a KdELECT assay. Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kd values were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1× PBS, 0.05% Tween 20). The beads were then re-suspended in elution buffer (1× PBS, 0.05% Tween 20, 0.5 μM nonbiotinylated affinity ligand) and incubated at room temperature with shaking for 30 minutes. The kinase concentration in the eluates was measured by qPCR.

### Part II—Results

Results showing ability of exemplary compounds to bind to EGFR are provided in Table 5 below. The symbol “++++” indicates a Kd less than 0.05 mM. The symbol “+++” indicates an Kd in the range of 0.05 mM to 0.5 mM. The symbol “++” indicates a Kd in the range of greater than 0.5 mM to 2.5 mM. The symbol “+” indicates a Kd greater than 2.5 mM. The symbol “N/A” indicates that no data was available.

TABLE 5

| Compound No. | Kd |
|---|---|
| I-28 | ++++ |
| I-33 | ++++ |
| I-34 | ++++ |
| I-66 | ++++ |
| I-67 | ++++ |
| I-69 | ++++ |
| I-112 | ++ |
| I-113 | ++ |
| I-115 | + |
| I-117 | + |
| I-118 | + |
| I-120 | + |
| I-121 | ++ |
| I-122 | +++ |
| I-123 | + |
| I-124 | + |
| I-125 | +++ |
| I-126 | ++ |
| I-127 | +++ |
| I-128 | +++ |
| I-129 | ++ |
| I-130 | ++ |
| I-131 | ++ |
| I-132 | +++ |
| I-133 | ++ |
| I-134 | ++++ |
| I-135 | ++++ |
| I-136 | ++++ |
| I-137 | ++++ |
| I-138 | ++++ |
| I-139 | +++ |
| I-140 | +++ |
| I-141 | +++ |
| I-143 | ++++ |
| I-146 | ++ |
| I-149 | ++ |
| I-150 | ++++ |
| I-152 | ++ |

### Example 140—Assay for Binding Affinity to HER2

Exemplary compounds were tested for ability to bind to HER2. Experimental procedures and results are provided below.

### Part I—Experimental Procedure

Compounds were tested using a KdELECT assay. Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kd values were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1× PBS, 0.05% Tween 20). The beads were then re-suspended in elution buffer (1× PBS, 0.05% Tween 20, 0.5 μM nonbiotinylated affinity ligand) and incubated at room temperature with shaking for 30 minutes. The kinase concentration in the eluates was measured by qPCR.

### Part II—Results

Results showing ability of exemplary compounds to bind to HER2 are provided in Table 6 below. The symbol “++++” indicates a Kd less than 0.05 mM. The symbol “+++” indicates a Kd in the range of 0.05 mM to 0.5 mM. The symbol “++” indicates a Kd in the range of greater than 0.5 mM to 2.5 mM. The symbol “+” indicates a Kd greater than 2.5 mM. The symbol “N/A” indicates that no data was available.

TABLE 6

| Compound No. | Kd |
|---|---|
| I-2 | ++++ |
| I-3 | ++++ |
| I-5 | ++++ |
| I-6 | ++++ |
| I-7 | ++++ |
| I-8 | ++++ |
| I-24 | ++++ |
| I-25 | ++++ |
| I-45 | ++++ |
| I-46 | ++++ |
| I-57 | ++++ |
| I-58 | ++++ |
| I-59 | ++++ |
| I-70 | ++++ |
| I-71 | ++++ |
| I-72 | ++++ |
| I-73 | ++++ |
| I-74 | ++++ |
| I-75 | ++++ |
| I-83 | ++++ |

### Example 141—Assay for Binding Affinity to BRD4-BD1

Exemplary compounds were tested for ability to bind to BRD4-BD1. Experimental procedures and results are provided below.

### Part I—Experimental Procedure

Compounds were tested using a bromoKdELECT assay. T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 μm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05% Tween 20, 1 mM DTT) to remove unbound ligand and to reduce nonspecific phage binding. Binding reactions were assembled by combining bromodomains, liganded affinity beads, and test compounds in 1× binding buffer (17% SeaBlock, 0.33×PBS, 0.04% Tween 20, 0.02% BSA, 0.004% Sodium azide, 7.4 mM DTT). Test compounds were prepared as 1000× stocks in 100% DMSO. Kds were determined using an 11-point 3-fold compound dilution series with one DMSO control point. All compounds for Kd measurements are distributed by acoustic transfer (non-contact dispensing) in 100% DMSO. The compounds were then diluted directly into the assays such that the final concentration of DMSO was 0.09%. All reactions performed in polypropylene 384-well plates. Each was a final volume of 0.02 ml. The assay plates were incubated at room temperature with shaking for 1 hour and the affinity beads were washed with wash buffer (1× PBS, 0.05% Tween 20). The beads were then resuspended in elution buffer (1× PBS, 0.05% Tween 20, 2 μM non-biotinylated affinity ligand) and incubated at room temperature with shaking for 30 minutes. The bromodomain concentration in the eluates was measured by qPCR.

### Part II—Results

Results showing ability of exemplary compounds to bind to BRD4-BD1 are provided in Table 7 below. The symbol “++++” indicates a Kd less than 0.05 mM. The symbol “+++” indicates a Kd in the range of 0.05 mM to 0.5 mM. The symbol “++” indicates a Kd in the range of greater than 0.5 mM to 2.5 mM. The symbol “+” indicates a Kd greater than 2.5 mM. The symbol “N/A” indicates that no data was available.

TABLE 7

| Compound No. | Kd |
|---|---|
| I-45 | ++++ |
| I-46 | ++++ |
| I-48 | +++ |
| I-50 | +++ |
| I-52 | +++ |
| I-55 | ++++ |
| I-70 | ++++ |
| I-71 | + |
| I-72 | ++++ |

### Example 142—Assay for Binding Affinity to BRD4-BD2

Exemplary compounds were tested for ability to bind to BRD4-BD2. Experimental procedures and results are provided below.

### Part I—Experimental Procedure

Compounds were tested using a bromoKdELECT assay. T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 μm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05% Tween 20, 1 mM DTT) to remove unbound ligand and to reduce nonspecific phage binding. Binding reactions were assembled by combining bromodomains, liganded affinity beads, and test compounds in 1× binding buffer (17% SeaBlock, 0.33×PBS, 0.04% Tween 20, 0.02% BSA, 0.004% Sodium azide, 7.4 mM DTT). Test compounds were prepared as 1000× stocks in 100% DMSO. Kds were determined using an 11-point 3-fold compound dilution series with one DMSO control point. All compounds for Kd measurements are distributed by acoustic transfer (non-contact dispensing) in 100% DMSO. The compounds were then diluted directly into the assays such that the final concentration of DMSO was 0.09%. All reactions performed in polypropylene 384-well plates. Each was a final volume of 0.02 ml. The assay plates were incubated at room temperature with shaking for 1 hour and the affinity beads were washed with wash buffer (1× PBS, 0.05% Tween 20). The beads were then resuspended in elution buffer (1× PBS, 0.05% Tween 20, 2 μM non-biotinylated affinity ligand) and incubated at room temperature with shaking for 30 minutes. The bromodomain concentration in the eluates was measured by qPCR.

### Part II—Results

Results showing ability of exemplary compounds to bind to BRD4-BD2 are provided in Table 8 below. The symbol “++++” indicates a Kd less than 0.05 mM. The symbol “+++” indicates a Kd in the range of 0.05 mM to 0.5 mM. The symbol “++” indicates a Kd in the range of greater than 0.5 mM to 2.5 mM. The symbol “+” indicates a Kd greater than 2.5 mM. The symbol “N/A” indicates that no data was available.

TABLE 8

| Compound No. | Kd |
|---|---|
| I-45 | ++++ |
| I-46 | ++++ |
| I-48 | ++++ |
| I-50 | ++++ |
| I-52 | ++++ |
| I-53 | + |
| I-55 | +++ |
| I-70 | ++++ |
| I-71 | + |
| I-72 | ++++ |

### Example 143—Assay for Binding Affinity to CDK1

Exemplary compounds were tested for ability to bind to CDK1. Experimental procedures and results are provided below.

### Part I—Experimental Procedure

Compounds were tested using a KdELECT assay. Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kd values were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1× PBS, 0.05% Tween 20). The beads were then re-suspended in elution buffer (1× PBS, 0.05% Tween 20, 0.5 μM nonbiotinylated affinity ligand) and incubated at room temperature with shaking for 30 minutes. The kinase concentration in the eluates was measured by qPCR.

### Part II—Results

Results showing ability of exemplary compounds to bind to CDK1 are provided in Table 9 below. The symbol “++++” indicates a Kd less than 0.05 mM. The symbol “+++” indicates a Kd in the range of 0.05 mM to 0.5 mM. The symbol “++” indicates a Kd in the range of greater than 0.5 mM to 2.5 mM. The symbol “+” indicates a Kd greater than 2.5 mM. The symbol “N/A” indicates that no data was available.

TABLE 9

| Compound No. | Kd |
|---|---|
| I-24 | +++ |
| I-25 | ++++ |
| I-27 | ++++ |
| I-28 | +++ |
| I-30 | +++ |
| I-33 | ++++ |
| I-34 | ++++ |
| I-35 | +++ |
| 1-37 | +++ |
| I-38 | +++ |
| I-40 | +++ |
| I-41 | +++ |
| I-42 | +++ |
| I-74 | ++++ |

### Example 144—Assay for Inhibition of KIF11

Exemplary compounds were tested for ability to inhibit KIF11. Experimental procedures and results are provided below.

### Part I—Experimental Procedure

The ATPase rate for Kif11 was monitored using an enzyme-coupled assay. Porcine brain microtubules (Cat #MT002) were purchased from Cytoskeleton (Denver, CO) and polymerized as per manufacturer's instructions and stored at 1 mg/ml at −80° C. GST-tagged Kifll/Eg5 (Cat # EGO1-XL) and the Kinesin ELIPA Biochem Kit (Cat # BK060) was also purchased from Cytoskeleton. Taxol, microtubules, 7-methylthioguanosine (MESG), and purine nucleoside phosphorylase (PNP) were added to final concentrations of 15 mM, 0.05 mg/mL, 200 mM, lU/m, 1 mM in EPLIA Reaction Buffer and allowed to incubate at room temperature for 15 minutes under rocking. Kif11 was then added at a final concentration of 0.025 mM and allowed to incubate at room temperature for an additional 15 minutes. Master mix (18.5 mL) was added to a black 384-well plate (Corning 3575), to which 0.5 mL of DMSO or compound were added to appropriate wells. Plates were incubated at room temperature for 2 hours. ATPase reactions were initiated by addition of 1 mM ATP and monitored by OD340 every 1 minute over the course of 2 hours. The rate of the reaction was taken from the linear part of the resulting curve. IC₅₀ was determined by plotting inhibitor concentration v. ATPase rate.

### Part II—Results

Results showing inhibition of KIF11 by exemplary compounds are provided in Table 10 below. The symbol “++++” indicates a IC₅₀ less than 0.05 mM. The symbol “+++” indicates an IC₅₀ in the range of 0.05 mM to 0.5 mM. The symbol “++” indicates a IC₅₀ in the range of greater than 0.5 mM to 2.5 mM. The symbol “+” indicates a IC₅₀ greater than 2.5 mM. The symbol “N/A” indicates that no data was available.

TABLE 10

| Compound No. | IC₅₀ |
|---|---|
| I-77 | ++++ |
| I-79 | ++++ |

### Example 145—Assay for Binding Affinity to mTOR

Exemplary compounds were tested for ability to bind to mTOR. Experimental procedures and results are provided below.

### Part I—Experimental Procedure

Compounds were tested using a KdELECT assay. Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kd values were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1× PBS, 0.05% Tween 20). The beads were then re-suspended in elution buffer (1× PBS, 0.05% Tween 20, 0.5 μM nonbiotinylated affinity ligand) and incubated at room temperature with shaking for 30 minutes. The kinase concentration in the eluates was measured by qPCR.

### Part II—Results

Results showing ability of exemplary compounds to bind to mTOR are provided in Table 11 below. The symbol “++++” indicates a Kd less than 0.05 mM. The symbol “+++” indicates a Kd in the range of 0.05 mM to 0.5 mM. The symbol “++” indicates a Kd in the range of greater than 0.5 mM to 2.5 mM. The symbol “+” indicates a Kd greater than 2.5 mM. The symbol “N/A” indicates that no data was available.

TABLE 11

| Compound No. | Kd |
|---|---|
| I-2 | ++++ |
| I-3 | ++++ |
| I-5 | ++++ |
| I-6 | ++++ |
| I-7 | ++++ |
| I-8 | ++++ |
| I-11 | + |
| I-13 | +++ |
| I-14 | ++ |
| I-16 | +++ |
| I-18 | ++++ |
| I-19 | ++++ |
| I-22 | ++++ |
| I-75 | ++++ |
| I-83 | ++++ |

### Example 146—Additional Assay for Binding Affinity to EGFR

Exemplary compounds were tested for ability to bind to EGFR WT (amino acids 669-1011, Accession Number NP_005219.2) or EGFR T790M, L858R (amino acids 669-1011 Accession Number NP_005219.2) expressed from bacteria or mammalian cells, respectively. Compounds were tested using the KdELECT assay at Eurofins Discovery. Experimental procedures and results are provided below.

### Part I—Experimental Procedure

Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kd values were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1× PBS, 0.05% Tween 20). The beads were then re-suspended in elution buffer (1× PBS, 0.05% Tween 20, 0.5 μM nonbiotinylated affinity ligand) and incubated at room temperature with shaking for 30 minutes. The kinase concentration in the eluates was measured by qPCR.

### Part II—Results

Results showing compound binding to EGFR are provided in Table 12 below. The symbol “++++” indicates a Kd less than 0.05 μM. The symbol “+++” indicates an Kd in the range of 0.05 μM to 0.5 μM. The symbol “++” indicates a Kd in the range of greater than 0.5 M to 2.5 μM. The symbol “+” indicates a Kd greater than 2.5 μM. The symbol “N/A” indicates that no data was available.

TABLE 12

| Compound | EGFR | EGFR |
|---|---|---|
| No. | DM Kd | WT Kd |
| I-28 | ++++ | +++ |
| I-29 | ++++ | +++ |
| I-33 | ++++ | +++ |
| I-32 | ++++ | +++ |
| I-34 | ++++ | +++ |
| I-67 | ++++ | +++ |
| I-68 | ++++ | ++ |
| I-69 | ++++ | ++ |
| I-112 | N/A | ++ |
| I-113 | N/A | ++ |
| I-115 | +++ | + |
| I-117 | +++ | + |
| I-118 | +++ | + |
| I-120 | +++ | + |
| I-121 | +++ | ++ |
| I-122 | ++++ | ++ |
| I-123 | ++++ | + |
| I-124 | ++++ | + |
| I-125 | ++++ | +++ |
| I-126 | +++ | ++ |
| I-127 | ++++ | +++ |
| I-128 | ++++ | +++ |
| I-129 | +++ | ++ |
| I-130 | ++++ | ++ |
| I-131 | ++++ | ++ |
| I-132 | ++++ | +++ |
| I-133 | ++++ | ++ |
| I-134 | ++++ | ++++ |
| I-135 | ++++ | ++++ |
| I-136 | ++++ | ++++ |
| I-137 | ++++ | ++++ |
| I-138 | ++++ | ++++ |
| I-139 | ++++ | +++ |
| I-140 | ++++ | +++ |
| I-141 | ++++ | +++ |
| I-143 | ++++ | ++++ |
| I-146 | ++++ | ++ |
| I-149 | ++++ | ++ |
| I-150 | ++++ | ++++ |
| I-152 | ++++ | ++ |

### Example 147—Assay for Binding Affinity to CDK9

Exemplary compounds were tested for ability to bind to full-length CDK9 (amino acids 1-372; Accession Number NP_001252.1) expressed from bacteria. Compounds were tested using a KdELECT assay. Experimental procedures and results are provided below.

### Part I—Experimental Procedure

Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kd values were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM nonbiotinylated affinity ligand) and incubated at room temperature with shaking for 30 minutes. The kinase concentration in the eluates was measured by qPCR.

### Part II—Results

Results showing ability of exemplary compounds to bind CDK9 are provided in Table 13 below. The symbol “++++” indicates a Kd less than 0.05 μM. The symbol “+++” indicates an Kd in the range of 0.05 μM to 0.5 μM. The symbol “++” indicates a Kd in the range of greater than 0.5 μM to 2.5 μM. The symbol “+” indicates a Kd greater than 2.5 μM. The symbol “N/A” indicates that no data was available.

TABLE 13

| Compound No. | Kd |
|---|---|
| I-33 | ++ |
| I-86 | ++++ |
| I-132 | ++++ |
| I-90 | ++++ |
| I-138 | ++ |
| I-143 | ++ |
| I-92 | + |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I 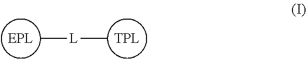
or a pharmaceutically acceptable salt thereof; wherein:
EPL is a moiety that binds to an effector protein selected from mTOR, PLK1, CDK1, CDK2, CDK9, BRD4, AURKA, AURKB, MEK, Src, c-KIT, KIF11, HSP90, tubulin, proteasome, topoisomerase, or HDAC;
L is a linker; and
TPL is a moiety that binds to a target protein selected from KRAS, HER2, and EGFR.

2. The compound of claim 1, wherein the compound is a compound of Formula I.

3. The compound of claim 1 or 2, wherein the EPL is a moiety that binds to mTOR.

4. The compound of claim 1 or 2, wherein the EPL has the following formula: 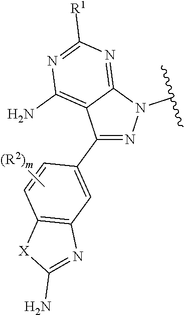
wherein:
R¹ and each R² represent independently for each occurrence halo, C₁-C₆ alkyl, C₁-C₆ haloalkyl, C₃-C₆ cycloalkyl, hydroxyl, C₁-C₆ alkoxy, or cyano;
R³ is hydrogen, C₁-C₆ alkyl, or C₃-C₆ cycloalkyl;
X is O, S, or N(R³); and
m is 0, 1, 2, or 3.

5. The compound of claim 1 or 2, wherein the EPL has the following formula: 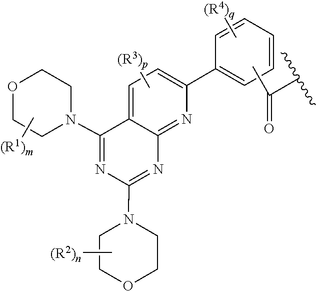
wherein:
R¹ and R² each represent independently for each occurrence halo, C₁-C₆ alkyl, C₁-C₆ haloalkyl, or C₃-C₆ cycloalkyl;
R³ and R⁴ each represent independently for each occurrence halo, C₁-C₆ alkyl, C₁-C₆ haloalkyl, C₃-C₆ cycloalkyl, hydroxyl, C₁-C₆ alkoxy, or cyano;
m, n, and q each represent independently 0, 1, 2, or 3; and
p is 0, 1, or 2.

6. The compound of claim 1 or 2, wherein the EPL is one of the following:

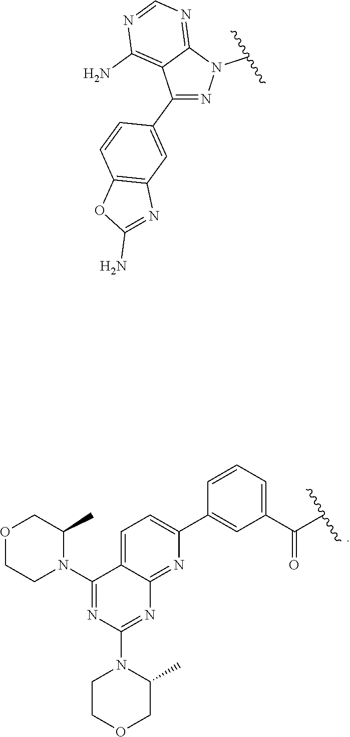 [SMILES: *n1nc(-c2ccc3oc(N)nc3c2)c2c(N)ncnc21 ; *C(=O)c1cccc(-c2ccc3c(N4CCOC[C@H]4C)nc(N4CCOC[C@H]4C)nc3n2)c1]

7. The compound of claim 1 or 2, wherein the EPL is a moiety that binds to PLK1.

8. The compound of claim 1 or 2, wherein the EPL has the following formula: 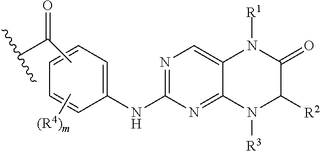
wherein:
R¹ and R² each represent independently C₁-C₆ alkyl, C₁-C₆ haloalkyl, or hydrogen;
R³ is C₃-C₆ cycloalkyl, C₁-C₆ alkyl, C₁-C₆ haloalkyl, or hydrogen;
R⁴ represents independently for each occurrence halo, C₁-C₆ alkyl, C₁-C₆ haloalkyl, C₃-C₆ cycloalkyl, hydroxyl, C₁-C₆ alkoxy, or cyano; and
m is 0, 1, 2, or 3.

9. The compound of claim 1 or 2, wherein the EPL is

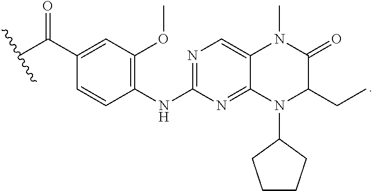 [SMILES: *C(=O)c1ccc(Nc2ncc3c(n2)N(C2CCCC2)C(CC)C(=O)N3C)c(OC)c1]

10. The compound of claim 1 or 2, wherein the EPL has the following formula: 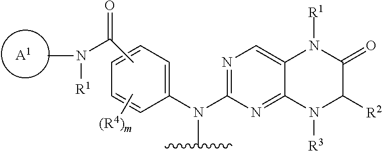
wherein:
R¹ and R² each represent independently for each occurrence C₁-C₆ alkyl, C₁-C₆ haloalkyl, or hydrogen;
R³ is C₃-C₆ cycloalkyl, C₁-C₆ alkyl, C₁-C₆ haloalkyl, or hydrogen;
R⁴ represents independently for each occurrence halo, C₁-C₆ alkyl, C₁-C₆ haloalkyl, C₃-C₆ cycloalkyl, hydroxyl, C₁-C₆ alkoxy, or cyano;
A¹ is a 3-7 membered saturated heterocyclyl containing 1, 2, or 3 heteroatoms independently selected from nitrogen, oxygen, and sulfur, wherein the heterocyclyl is optionally substituted with 1 or 2 occurrences of R⁴; and
m is 0, 1, 2, or 3.

11. The compound of claim 1 or 2, wherein the EPL has the following formula: 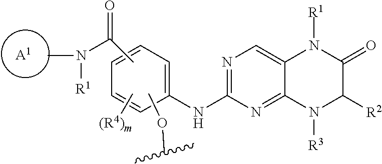
wherein:
R¹ and R² each represent independently for each occurrence C₁-C₆ alkyl, C₁-C₆ haloalkyl, or hydrogen;
R³ is C₃-C₆ cycloalkyl, C₁-C₆ alkyl, C₁-C₆ haloalkyl, or hydrogen;
R⁴ represents independently for each occurrence halo, C₁-C₆ alkyl, C₁-C₆ haloalkyl, C₃-C₆ cycloalkyl, hydroxyl, C₁-C₆ alkoxy, or cyano;
A¹ is a 3-7 membered saturated heterocyclyl containing 1, 2, or 3 heteroatoms independently selected from nitrogen, oxygen, and sulfur, wherein the heterocyclyl is optionally substituted with 1 or 2 occurrences of R⁴; and
m is 0, 1, 2, or 3.

12. The compound of claim 1 or 2, wherein the EPL is one of the following:

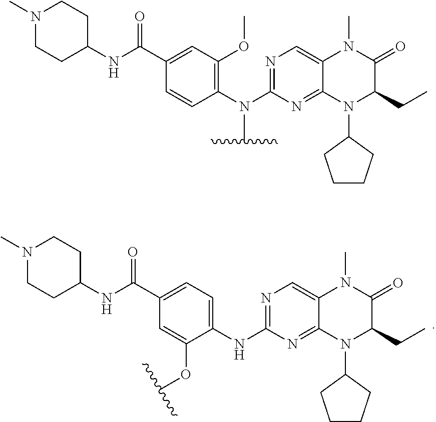

13. The compound of claim 1 or 2, wherein the EPL is a moiety that binds to CDK1.

14. The compound of claim 1 or 2, wherein the EPL has the following formula: 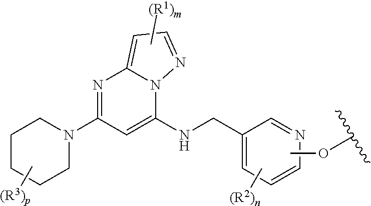
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

15. The compound of claim 1 or 2, wherein the EPL has the following formula: 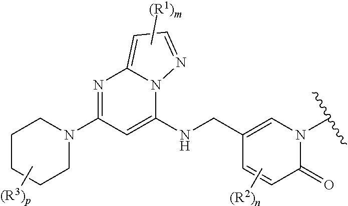
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

16. The compound of claim 1 or 2, wherein the EPL has the following formula: 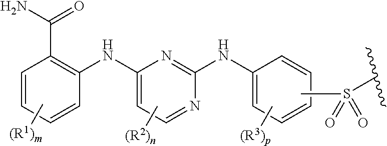
wherein:
R¹, R² and R³ each represent independently for each occurrence halo, C₁-C₆ alkyl, C₁-C₆ haloalkyl, C₃-C₆ cycloalkyl, hydroxyl, C₁-C₆ alkoxy, or cyano;
m and p each represent independently 0, 1, or 2; and
n is 0, 1 or 2.

17. The compound of claim 1 or 2, wherein the EPL is one of the following:

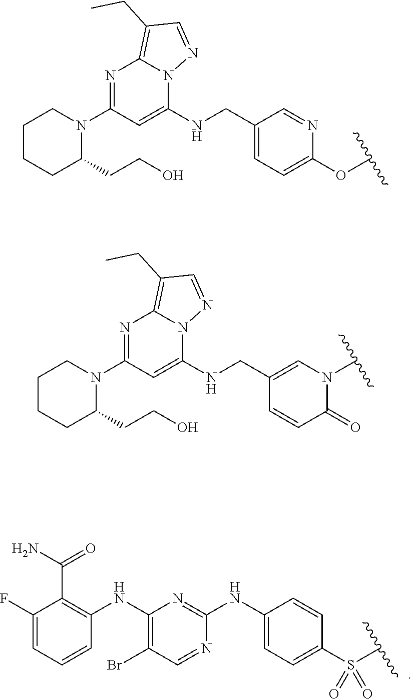 [SMILES: *Oc1ccc(CNc2cc(N3CCCC[C@H]3CCO)nc3c(CC)cnn23)cn1 ; *n1cc(CNc2cc(N3CCCC[C@H]3CCO)nc3c(CC)cnn23)ccc1=O ; *S(=O)(=O)c1ccc(Nc2ncc(Br)c(Nc3cccc(F)c3C(N)=O)n2)cc1]

18. The compound of claim 1 or 2, wherein the EPL is one of the following:

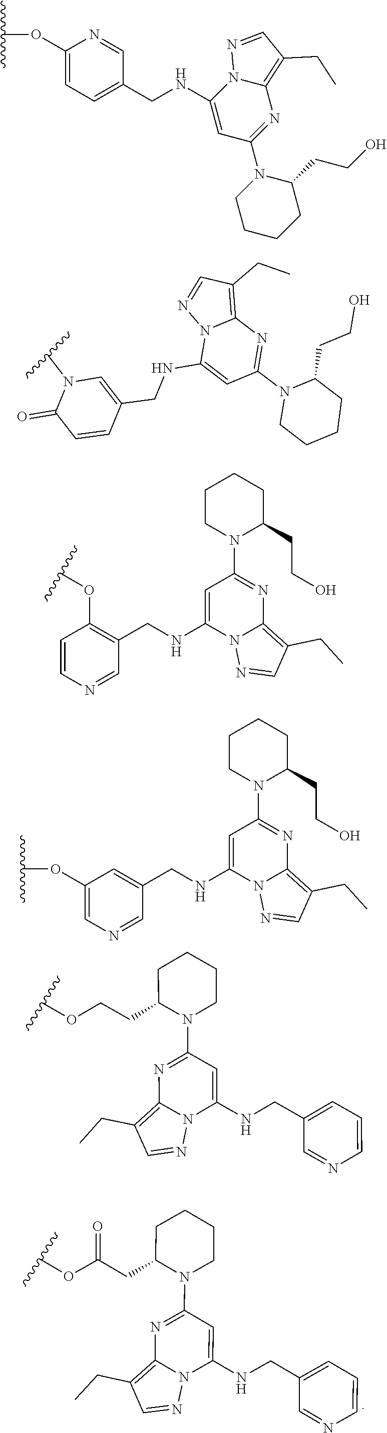

19. The compound of claim 1 or 2, wherein the EPL is a moiety that binds to CDK2.

20. The compound of claim 1 or 2, wherein the EPL has the following formula: 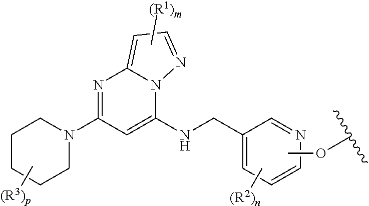
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

21. The compound of claim 1 or 2, wherein the EPL has the following formula: 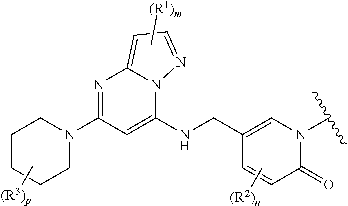
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

22. The compound of claim 1 or 2, wherein the EPL is one of the following:

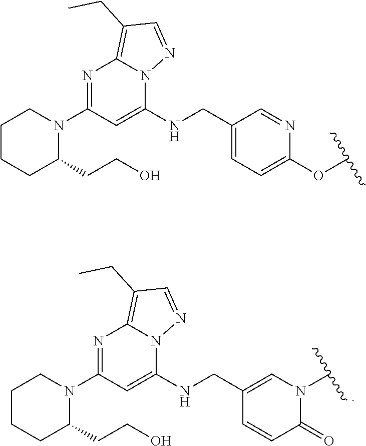 [SMILES: *Oc1ccc(CNc2cc(N3CCCC[C@H]3CCO)nc3c(CC)cnn23)cn1 ; *n1cc(CNc2cc(N3CCCC[C@H]3CCO)nc3c(CC)cnn23)ccc1=O]

23. The compound of claim 1 or 2, wherein the EPL is a moiety that binds to CDK9.

24. The compound of claim 1 or 2, wherein the EPL has the following formula: 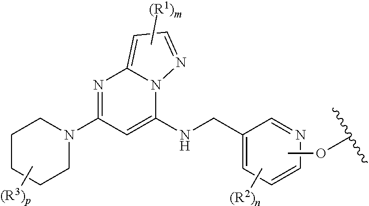
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

25. The compound of claim 1 or 2, wherein the EPL has the following formula: 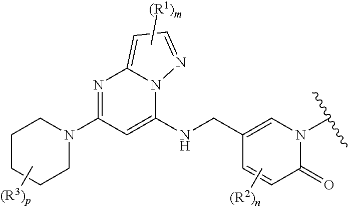
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

26. The compound of claim 1 or 2, wherein the EPL is one of the following:

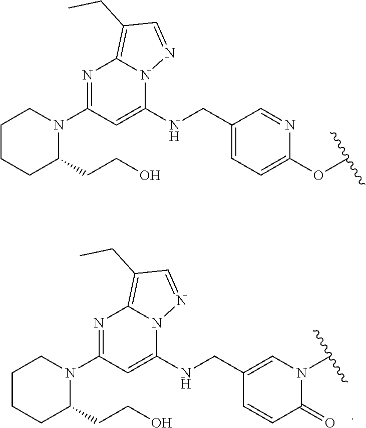 [SMILES: *Oc1ccc(CNc2cc(N3CCCC[C@H]3CCO)nc3c(CC)cnn23)cn1 ; *n1cc(CNc2cc(N3CCCC[C@H]3CCO)nc3c(CC)cnn23)ccc1=O]

27. The compound of claim 1 or 2, wherein the EPL has the following formula: 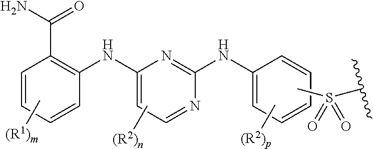
wherein:
R¹, R² and R³ each represent independently for each occurrence halo, C₁-C₆ alkyl, C₁-C₆ haloalkyl, C₃-C₆ cycloalkyl, hydroxyl, C₁-C₆ alkoxy, or cyano;
m and p each represent independently 0, 1, or 2; and
n is 0, 1 or 2.

28. The compound of claim 1 or 2, wherein EPL is one of the following:

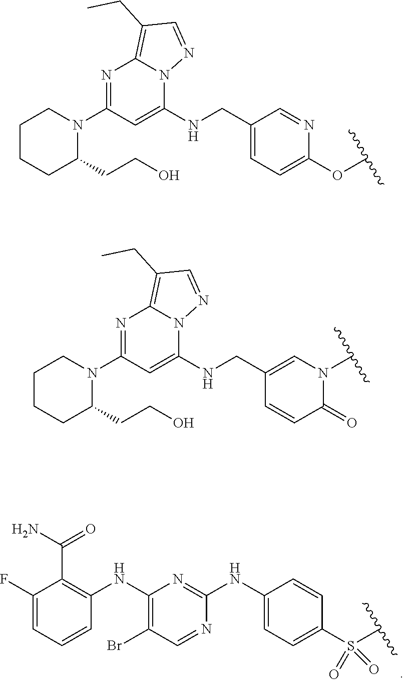 [SMILES: *Oc1ccc(CNc2cc(N3CCCC[C@H]3CCO)nc3c(CC)cnn23)cn1 ; *n1cc(CNc2cc(N3CCCC[C@H]3CCO)nc3c(CC)cnn23)ccc1=O ; *S(=O)(=O)c1ccc(Nc2ncc(Br)c(Nc3cccc(F)c3C(N)=O)n2)cc1]

29. The compound of claim 1 or 2, wherein the EPL is a moiety that binds to BRD4.

30. The compound of claim 1 or 2, wherein the EPL has the following formula: 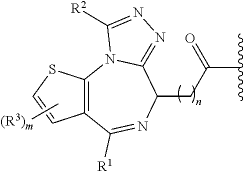
wherein:
R¹ is phenyl, C₃-C8 cycloalkyl, or 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano;
R² and each R³ represent independently for each occurrence halo, C₁-C₆ alkyl, C₁-C₆ haloalkyl, C₃-C₆ cycloalkyl, hydroxyl, C₁-C₆ alkoxy, or cyano;
m is 0, 1, or 2; and
n is 0, 1, 2, 3, or 4.

31. The compound of claim 1 or 2, wherein the EPL is

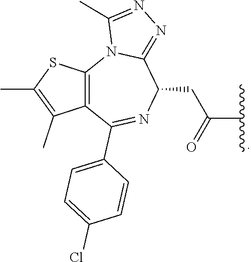 [SMILES: *C(=O)C[C@@H]1N=C(c2ccc(Cl)cc2)c2c(sc(C)c2C)-n2c(C)nnc21]

32. The compound of claim 1 or 2, wherein the EPL is a moiety that binds to AURKA.

33. The compound of claim 1 or 2, wherein the EPL has the following formula: 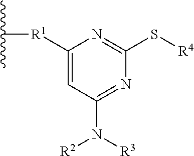
wherein:
R¹ is 4-7 membered, saturated heterocyclylene containing 1, 2, or 3 heteroatoms independently selected from nitrogen, oxygen, and sulfur;
R² is a 5-6membered heteroaryl containing 1, 2, or 3 heteroatoms independently selected from nitrogen, oxygen, and sulfur; wherein the heteroaryl is optionally substituted with 1 or 2 substituents independently selected from halo, C₁-C₆ alkyl, C₁-C₆ haloalkyl, C₃-C₆ cycloalkyl, hydroxyl, C₁-C₆ alkoxy, or cyano;
R³ represents independently for each occurrence H or C₁-C₆ alkyl; and
R⁴ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from —N(R³)C(O)—(C₃-C₆ cycloalkyl), —N(R³)C(O)—(C₁-C₆ alkyl), halo, C₁-C₆ alkyl, C₁-C₆ haloalkyl, C₃-C₆ cycloalkyl, hydroxyl, C₁-C₆ alkoxy, or cyano.

34. The compound of claim 1 or 2, wherein the EPL has the following formula: 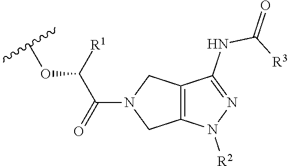
wherein:
R¹ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano,
R² is -(phenylene)-(4-7 membered, saturated heterocyclyl containing 1, 2, or 3 heteroatoms independently selected from nitrogen, oxygen, and sulfur); wherein the heterocyclyl is optionally substituted with 1 or 2 substituents independently selected from halo, C₁-C₆ alkyl, C₁-C₆ haloalkyl, C₃-C₆ cycloalkyl, hydroxyl, C₁-C₆ alkoxy, or cyano;
R³ represents independently for each occurrence H or C₁-C₆ alkyl.

35. The compound of claim 1 or 2, wherein the EPL is one of the following:

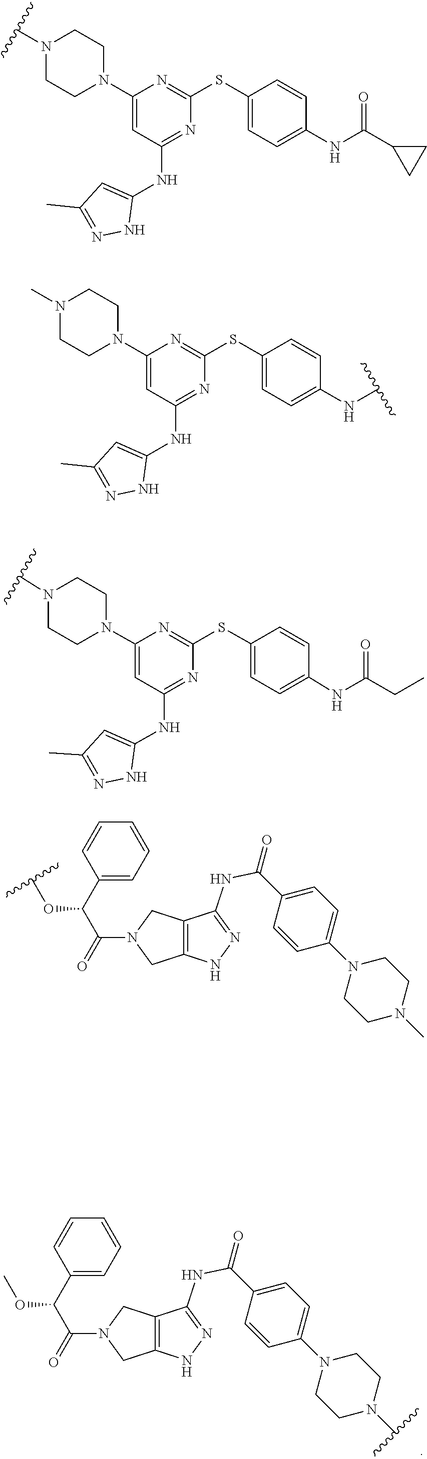

36. The compound of claim 1 or 2, wherein the EPL is a moiety that binds to AURKB.

37. The compound of claim 1 or 2, wherein the EPL has the following formula: 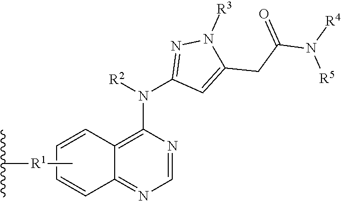
wherein:
R¹ is 4-10 membered heteroalkylene;
R², R³, and R⁵ are independently H or C₁-C₆ alkyl; and
R⁴ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano.

38. The compound of claim 1 or 2, wherein the EPL is one of the following:

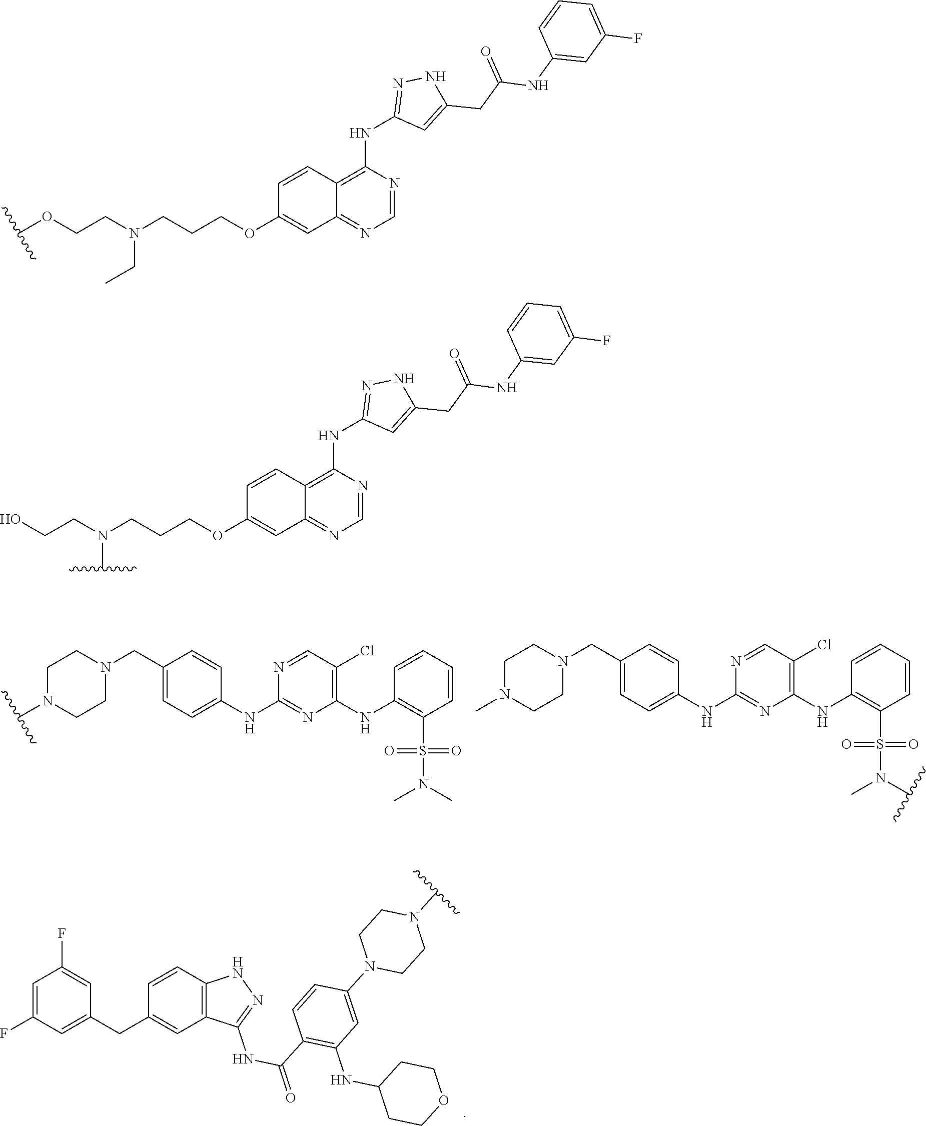

39. The compound of claim 1 or 2, wherein the EPL is a moiety that binds to MEK.

40. The compound of claim 1 or 2, wherein the EPL has the following formula: 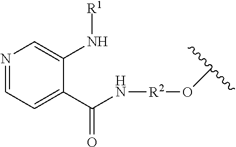
wherein:
R¹ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano; and
R² is —(C₂₋₆ alkylene optionally substituted by one hydroxyl).

41. The compound of claim 1 or 2, wherein the EPL is one of the following:

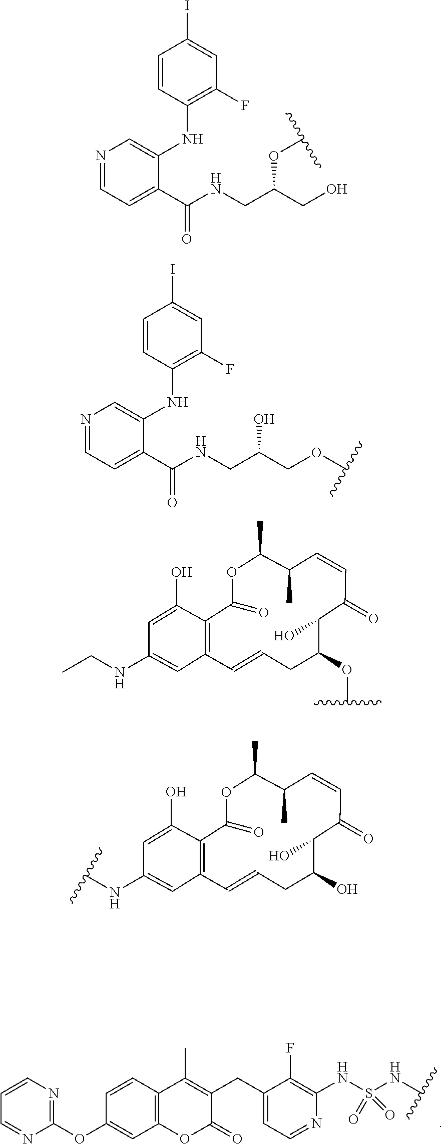

42. The compound of claim 1 or 2, wherein the EPL is a moiety that binds to Src.

43. The compound of claim 1 or 2, wherein the EPL is a moiety that binds to c-KIT.

44. The compound of claim 1 or 2, wherein the EPL is a moiety that binds to KIF11.

45. The compound of claim 1 or 2, wherein the EPL has the following formula: 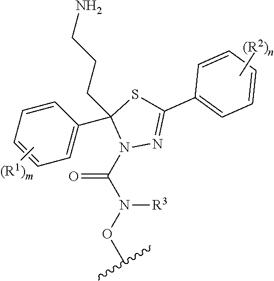
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ is H, C₁-C₆ alkyl, C₁-C₆ haloalkyl, or C₃-C₆ cycloalkyl; and
m and n each represent independently 0, 1, 2, or 3.

46. The compound of claim 1 or 2, wherein the EPL has the following formula: 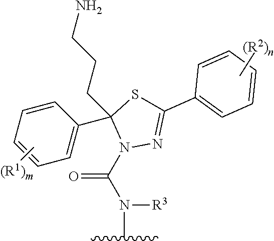
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ is H, C₁-C₆ alkyl, C₁-C₆ haloalkyl, or C₃-C₆ cycloalkyl; and
m and n each represent independently 0, 1, 2, or 3.

47. The compound of claim 1 or 2, wherein the EPL has the following formula: 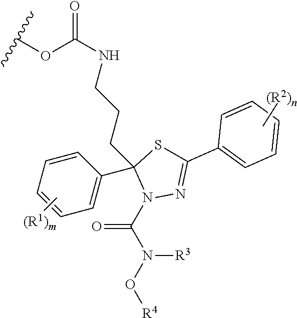
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ and R⁴ each represent independently H, C₁-C₆ alkyl, C₁-C₆ haloalkyl, or C₃-C₆ cycloalkyl; and
m and n each represent independently 0, 1, 2, or 3.

48. The compound of claim 1 or 2, wherein the EPL is one of the following:

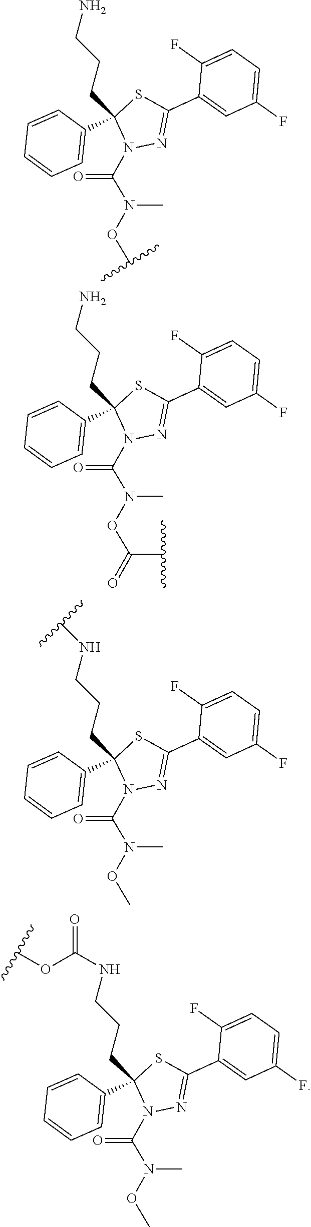 [SMILES: *ON(C)C(=O)N1N=C(c2cc(F)ccc2F)S[C@@]1(CCCN)c1ccccc1 ; *C(=O)ON(C)C(=O)N1N=C(c2cc(F)ccc2F)S[C@@]1(CCCN)c1ccccc1 ; *NCCC[C@@]1(c2ccccc2)SC(c2cc(F)ccc2F)=NN1C(=O)N(C)OC ; *OC(=O)NCCC[C@@]1(c2ccccc2)SC(c2cc(F)ccc2F)=NN1C(=O)N(C)OC]

49. The compound of claim 1 or 2, wherein the EPL is

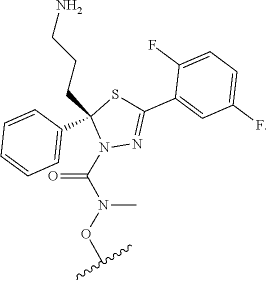 [SMILES: *ON(C)C(=O)N1N=C(c2cc(F)ccc2F)S[C@@]1(CCCN)c1ccccc1]

50. The compound of claim 1 or 2, wherein the EPL is or

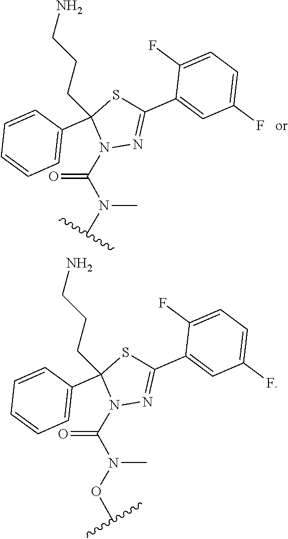

51. The compound of claim 1 or 2, wherein the EPL is a moiety that binds to HSP90.

52. The compound of claim 1 or 2, wherein the EPL is a moiety that binds to tubulin.

53. The compound of claim 1 or 2, wherein the EPL is a moiety that binds to proteasome.

54. The compound of claim 1 or 2, wherein the EPL is a moiety that binds to topoisomerase.

55. The compound of claim 1 or 2, wherein the EPL is a moiety that binds to HDAC.

56. The compound of any one of claims 1-55, wherein the TPL is a moiety that binds to KRAS.

57. The compound of any one of claims 1-55, wherein the TPL is one of the following: 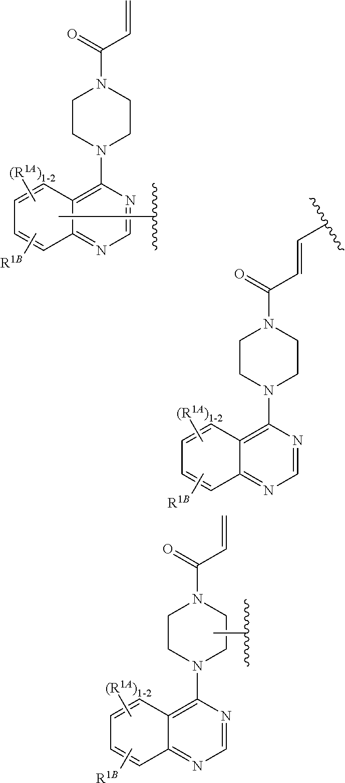
wherein:
R^{1A} represents independently for each occurrence hydrogen, halo, hydroxyl, C₁₋₄ alkyl, or C₁₋₄ alkoxyl; and
R^{1B} is C₆₋₁₂ aryl optionally substituted by 1, 2, or 3 substituents independently selected from halo, hydroxyl, C₁₋₄ alkyl, or C₁₋₄ alkoxyl.

58. The compound of any one of claims 1-55, wherein the TPL is one of the following:

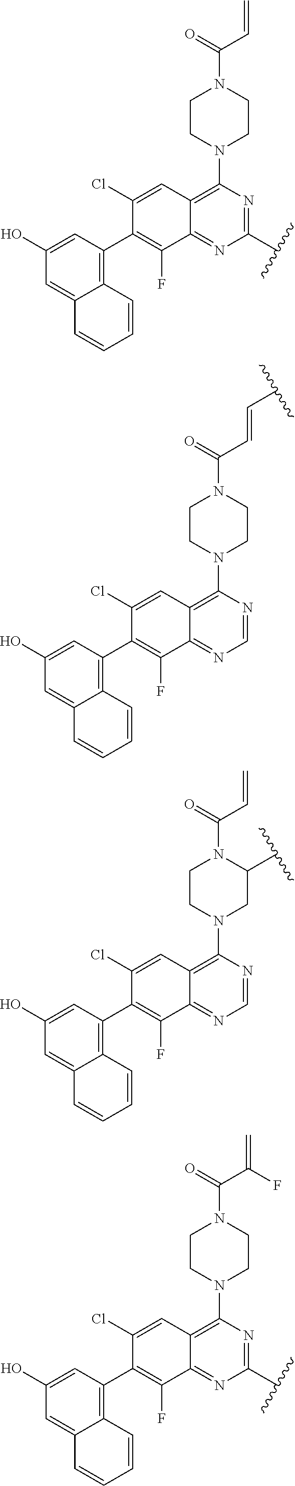 [SMILES: *c1nc(N2CCN(C(=O)C=C)CC2)c2cc(Cl)c(-c3cc(O)cc4ccccc34)c(F)c2n1 ; */C=C/C(=O)N1CCN(c2ncnc3c(F)c(-c4cc(O)cc5ccccc45)c(Cl)cc23)CC1 ; *C1CN(c2ncnc3c(F)c(-c4cc(O)cc5ccccc45)c(Cl)cc23)CCN1C(=O)C=C ; *c1nc(N2CCN(C(=O)C(=C)F)CC2)c2cc(Cl)c(-c3cc(O)cc4ccccc34)c(F)c2n1]

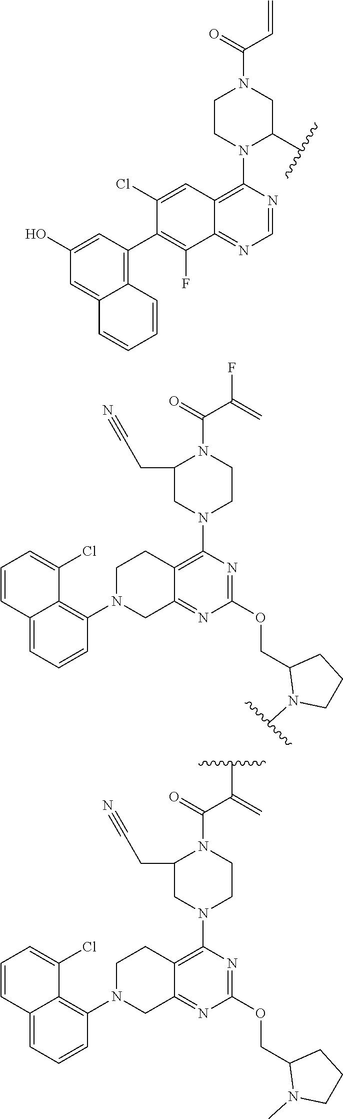

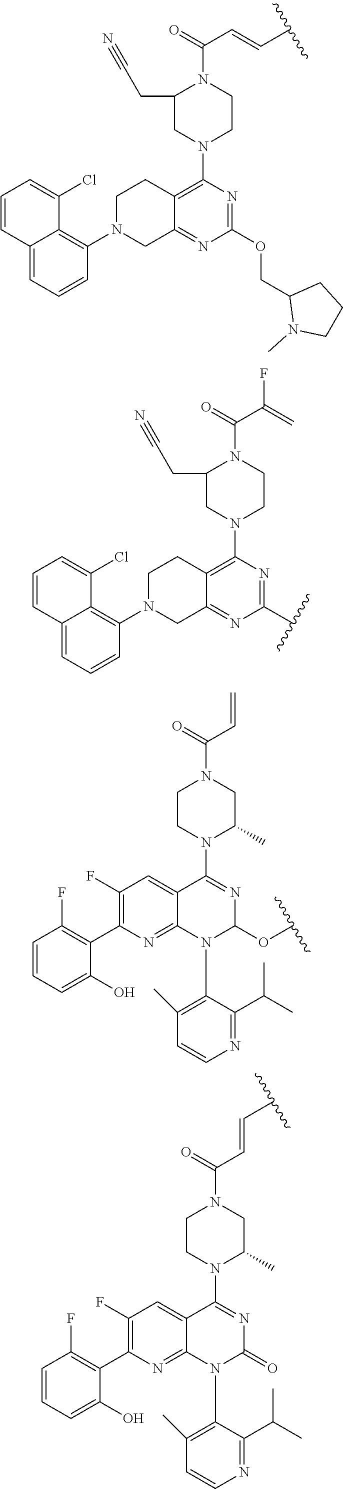

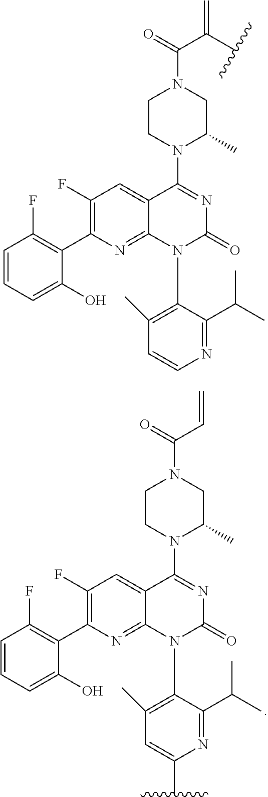

59. The compound of any one of claims 1-55, wherein the TPL is one of the following: 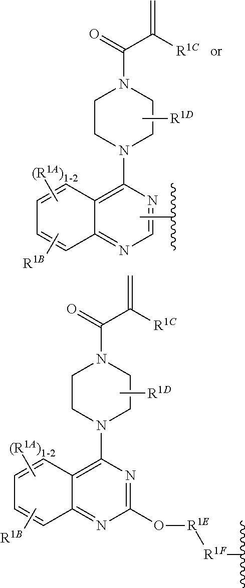
wherein:
R^{1A} represents independently for each occurrence hydrogen, halo, hydroxyl, C₁₋₄ alkyl, or C₁₋₄ alkoxyl;
R^{1B} is C₆₋₁₂ aryl optionally substituted by 1, 2, or 3 substituents independently selected from halo, hydroxyl, C₁₋₄ alkyl, or C₁₋₄ alkoxyl,
R^{1C} represents independently for each occurrence hydrogen, halo, or C₁₋₄ alkyl;
R^{1D} represents independently for each occurrence (C₁₋₆ alkylene)-CN;
R^{1E} is C₁₋₆ alkylene; and
R^{1F} is 3-6 membered saturated, monocyclic heterocyclylene containing 1, 2, or 3 heteroatoms independently selected from oxygen, nitrogen, and sulfur.

60. The compound of any one of claims 1-55, wherein the TPL is one of the following: 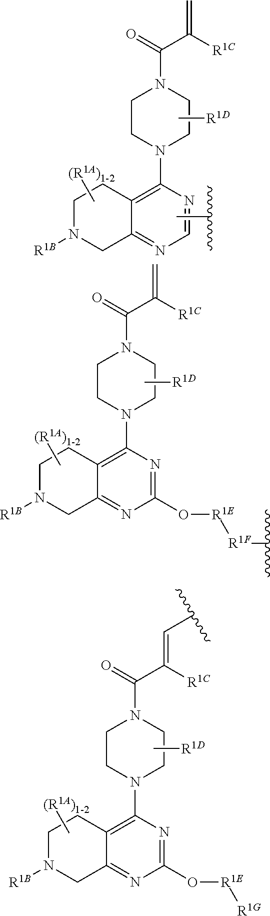
wherein:
R^{1A} represents independently for each occurrence hydrogen, halo, hydroxyl, C₁₋₄ alkyl, or C₁₋₄ alkoxyl;
R^{1B} is C₆₋₁₂ aryl optionally substituted by 1, 2, or 3 substituents independently selected from halo, hydroxyl, C₁₋₄ alkyl, or C₁₋₄ alkoxyl,
R^{1C} represents independently for each occurrence hydrogen, halo, or C₁₋₄ alkyl;
R^{1D} represents independently for each occurrence (C₁₋₆ alkylene)-CN;
R^{1E} is C₁₋₆ alkylene; and
R^{1F} is 3-6 membered saturated, monocyclic heterocyclylene containing 1, 2, or 3 heteroatoms independently selected from oxygen, nitrogen, and sulfur, wherein the heterocyclylene is substituted with 1 or 2 occurrences of R^{1A};
R^{1G} is 3-6 membered saturated, monocyclic heterocyclyl containing 1, 2, or 3 heteroatoms independently selected from oxygen, nitrogen, and sulfur, wherein the heterocyclyl is substituted with 1 or 2 occurrences of R^{1A}.

61. The compound of any one of claims 1-55, wherein the TPL is

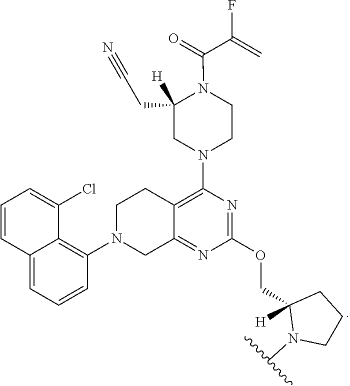 [SMILES: *N1CCC[C@H]1COc1nc2c(c(N3CCN(C(=O)C(=C)F)[C@@H](CC#N)C3)n1)CCN(c1cccc3cccc(Cl)c13)C2]

62. The compound of any one of claims 1-55, wherein the TPL has the following formula: 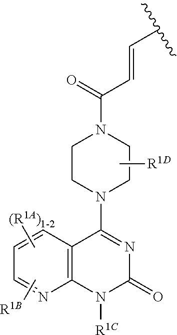
R^{1A} represents independently for each occurrence hydrogen, halo, hydroxyl, C₁₋₄ alkyl, or C₁₋₄ alkoxyl;
R^{1B} and R^{1C} are independently phenyl optionally substituted by 1, 2, or 3 substituents independently selected from halo, hydroxyl, C₁₋₄ alkyl, or C₁₋₄ alkoxyl;
R^{1D} is hydrogen or C₁₋₄ alkyl;

63. The compound of any one of claims 1-55, wherein the TPL is

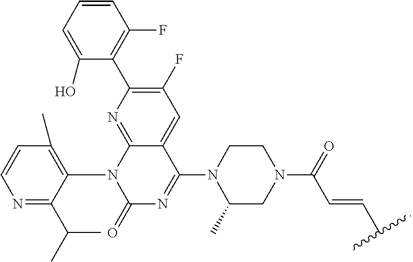 [SMILES: */C=C/C(=O)N1CCN(c2nc(=O)n(-c3c(C)ccnc3C(C)C)c3nc(-c4c(O)cccc4F)c(F)cc23)[C@@H](C)C1]

64. The compound of any one of claims 1-55, wherein the TPL is a moiety that binds to HER2.

65. The compound of any one of claims 1-55, wherein the TPL is one of the following 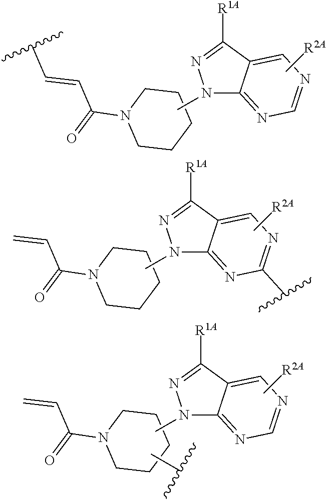
wherein:
R^{1A} is —C(O)(NR^{5A})-(phenyl optionally substituted with 1, 2, 3, 4, or 5 substituents independently selected from halo, hydroxyl, C₁₋₄ alkyl, C₁₋₄ alkoxyl, and —(C₁₋₄ alkylene-C(O)N(R^{5A})(R^{6A}));
R^{2A} is hydrogen, halo, hydroxyl, C₁₋₄ alkyl, C₁₋₄ alkoxyl, or —N(R^{5A})(R^{6A}); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to the same nitrogen atom are taken together with the nitrogen atom to which they are attached to form a 3-7 membered heterocyclic ring.

66. The compound of any one of claims 1-55, wherein the TPL is one of the following:

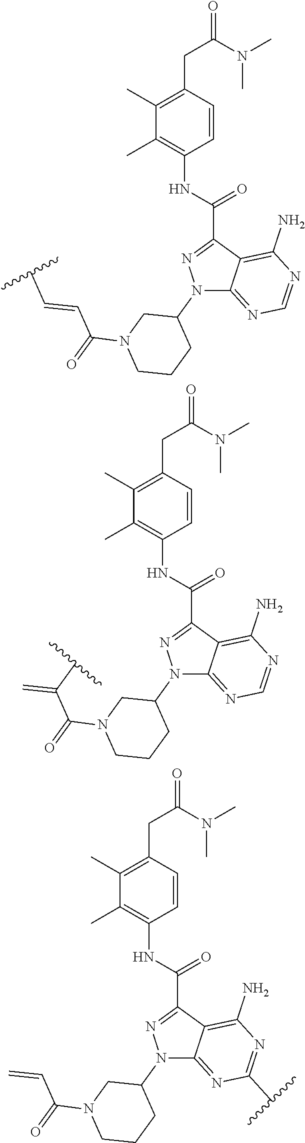

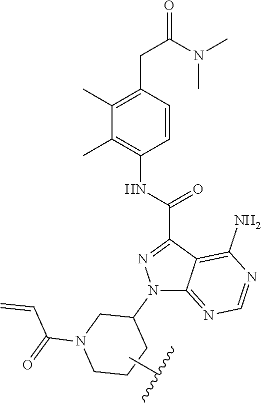

67. The compound of any one of claims 1-55, wherein the TPL is a moiety that binds to EGFR.

68. The compound of any one of claims 1-55, wherein the TPL is one of the following: 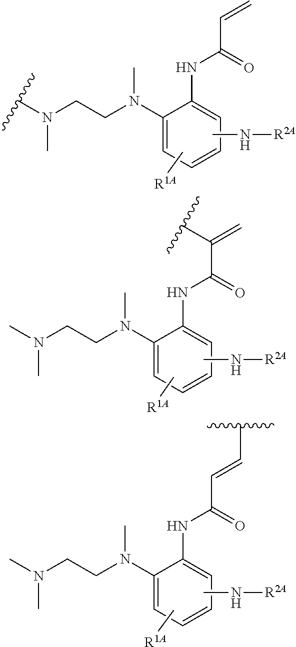
wherein:
R^{1A} is hydrogen, halo, hydroxyl, C₁₋₄ alkyl, C₁₋₄ alkoxyl, or N(R^{5A})(R^{6A}); and
R^{2A} is -(5-12 membered heteroaryl containing 1, 2, or 3 heteroatoms independently selected from nitrogen, oxygen and sulfur, wherein said heteroaryl is optionally substituted with 1, 2, 3, 4, or 5 substituents independently selected from halo, hydroxyl, C₁₋₄ alkyl, and C₁₋₄ alkoxyl)-(5-12 membered heteroaryl containing 1, 2, or 3 heteroatoms independently selected from nitrogen, oxygen and sulfur, wherein said heteroaryl is optionally substituted with 1, 2, 3, 4, or 5 substituents independently selected from halo, hydroxyl, C₁₋₄ alkyl, and C₁₋₄ alkoxyl); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to the same nitrogen atom are taken together with the nitrogen atom to which they are attached to form a 3-7 membered heterocyclic ring.

69. The compound of any one of claims 1-51, wherein the TPL is one of the following: 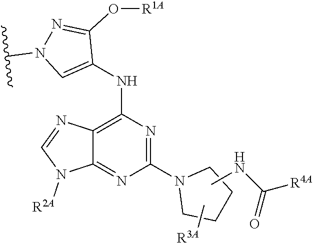
wherein R^{1A} is C₁₋₄ alkyl; R^{2A} is hydrogen or C₁₋₄ alkyl; R^{3A} is halo; and R^{4A} is C₂₋₆ alkenyl.

70. The compound of any one of claims 1-55, wherein the TPL is one of the following: 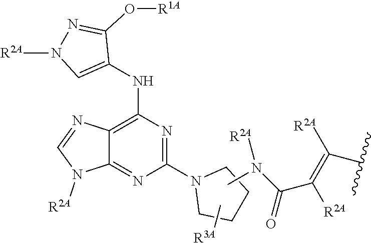
wherein R^{1A} is C₁₋₄ alkyl; R^{2A} represents independently for each occurrence hydrogen or C₁₋₄ alkyl; and R^{3A} is halo.

71. The compound of any one of claims 1-55, wherein the TPL is one of the following:

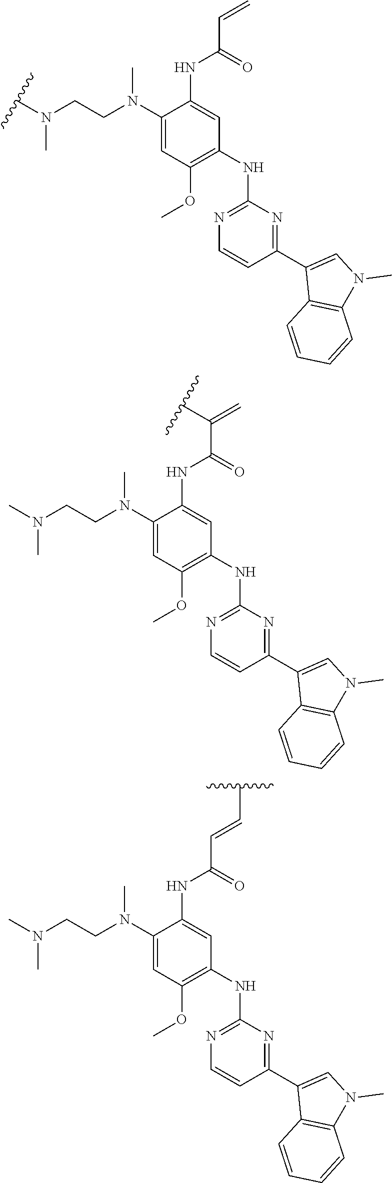

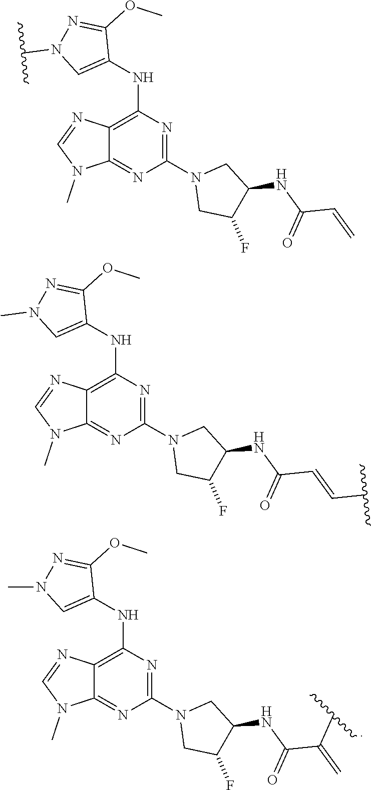

72. The compound of any one of claims 1-71, wherein L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

73. The compound of any one of claims 1-71, wherein L is a bivalent, saturated or unsaturated, straight or branched C₁₋₆₀ hydrocarbon chain, wherein (i) 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur, and (ii) 0-1 methylene units of the hydrocarbon are independently replaced with —C(O)—(C₂₋₆ alkenylene)-, —C(O)—(C₂₋₆ fluoroalkenylene)-, —C(O)—(C₂₋₆ alkynylene)-, —S(O)₂—(C₂₋₆ alkenylene)-, —S(O)₂—(C₂₋₆ fluoroalkenylene)-, —S(O)₂—(C₂₋₆ alkynylene)-, or —(C₁₋₆ alkylene substituted with one R^{WH})—, wherein R^{WH} is

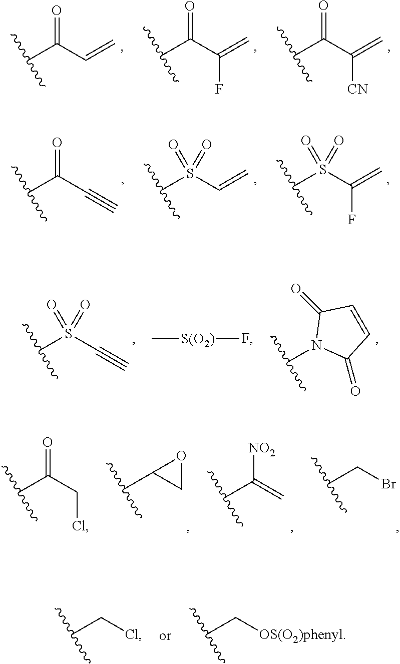

74. The compound of any one of claims 1-71, wherein L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, —C(O)N(C₁₋₆ alkyl)-, 3-10 membered carbocyclyl, or 3-10 membered heterocyclyl containing 1, 2, or 3 heteroatoms independently selected from nitrogen, oxygen, and sulfur.

75. The compound of any one of claims 1-71, wherein L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, or —C(O)N(C₁₋₆ alkyl)-.

76. The compound of any one of claims 1-71, wherein L has the formula —(C₀₋₁₂ alkylene)-(optionally substituted 3-40 membered heteroalkylene)-(C₀₋₁₂ alkylene)-.

77. The compound of any one of claims 1-71, wherein L is C₄₋₁₄ alkylene.

78. The compound of any one of claims 1-71, wherein L is —(CH₂)₆₋₁₀—.

79. The compound of any one of claims 1-71, wherein L is —CH₂CH₂(OCH₂CH₂)—***, —CH₂CH₂(OCH₂CH₂)₂—***, —CH₂CH₂(OCH₂CH₂)₃—***, —CH₂CH₂(OCH₂CH₂)₄—***, —CH₂CH₂(OCH₂CH₂)₅—***, —CH₂CH₂(OCH₂CH₂)₆—***, —CH₂CH₂(OCH₂CH₂)₇—***, —CH₂CH₂(OCH₂CH₂)₈—***, —CH₂CH₂(OCH₂CH₂)₉—***, —CH₂CH₂(OCH₂CH₂)₁₀—***, —CH₂CH₂(OCH₂CH₂)₁₁—***, —CH₂CH₂(OCH₂CH₂)₁₂—***, —CH₂CH₂(OCH₂CH₂)₁₃—***, —CH₂CH₂(OCH₂CH₂)₁₄—***, —CH₂CH₂(OCH₂CH₂)₁₅—***, or —CH₂CH₂(OCH₂CH₂)₁₆₋₂₀—***, where** * is a point of attachment to TPL.

80. The compound of any one of claims 1-71, wherein L is —(C₂₋₂₀ alkylene)-(OCH₂CH₂)₂₋₄—(C₀₋₄ alkylene)-***, —(C₂₋₂₀ alkylene)-(OCH₂CH₂)₅₋₇—(C₀₋₄ alkylene)-***, —(C₂₋₂₀ alkylene)-(OCH₂CH₂)₈₋₁₀—(C₀₋₄ alkylene)-***, —(C₂₋₂₀ alkylene)-(OCH₂CH₂)₁₁₋₁₃—(C₀₋₄ alkylene)-***, —(C₂₋₂₀ alkylene)-(OCH₂CH₂)₁₄₋₁₆—(C₀₋₄ alkylene)-***, —(C₂₋₂₀ alkylene)-(OCH₂CH₂)₁₇₋₂₀—(C₀₋₄ alkylene)-***, —(C₁₋₂₀ alkylene)-(OCH₂CH₂)₁₋₁₀—(C₀₋₄ alkylene)-C(O)—***, or —(C₁₋₂₀ alkylene)-(OCH₂CH₂)₁₁₋₂₀—(C₀₋₄ alkylene)-C(O)—***, where *** is a point of attachment to TPL.

81. The compound of any one of claims 1-71, wherein L is —O(CH₂CH₂O)_{2 -4}—(C₀₋₄ alkylene)-***, —O(CH₂CH₂O)₅₋₇—(C₀₋₄ alkylene)-***, —O(CH₂CH₂O)₈₋₁₀—(C₀₋₄ alkylene)-***, —O(CH₂CH₂O)₁₁₋₁₃—(C₀₋₄ alkylene)-***, —O(CH₂CH₂O)₁₄₋₁₆—(C₀₋₄ alkylene)-***, —O(CH₂CH₂O)₁₆₋₂₀—(C₀₋₄ alkylene)-***, —O(CH₂CH₂O)₂₋₁₀—(C₀₋₄ alkylene)C(O)—***, or —O(CH₂CH₂O)₁₁₋₂₀(C₀₋₄ alkylene)C(O)—***, where *** is a point of attachment to TPL.

82. The compound of any one of claims 1-71, wherein L is —(C₀₋₂₀ alkylene)-(OCH₂CH₂)₁₋₁₀—(N(C₁₋₄ alkyl))-***, —(C₀₋₂₀ alkylene)-(OCH₂CH₂)₁₁₋₂₀—(N(C₁₋₄ alkyl))-***, —(C₀₋₂₀ alkylene)-(CH₂CH₂O)₁₋₁₀—(C₂₋₁₀ alkylene)-(N(C₁₋₄ alkyl))—(C₀₋₁₀ alkylene)-***, or —(C₀₋₂₀ alkylene)-(CH₂CH₂O)₁₁₋₂₀—(C₂₋₁₀ alkylene)-(N(C₁₋₄ alkyl))—(C₀₋₁₀ alkylene)-***, where *** is a point of attachment to TPL.

83. The compound of any one of claims 1-71, wherein L is —N(H)—(C₂₋₉ alkylene)-O—(C₁₋₆ alkylene)-C(O)—***, —N(H)—(C₁₀₋₂₀ alkylene)-O—(C₁₋₆ alkylene)-C(O)—***, —N(H)—[(C₂₋₄ alkylene)-O—]₂₋₆—(C₁₋₆ alkylene)-C(O)—***, —N(H)—[(C₂₋₄ alkylene)-O—]₇₋₁₅—(C₁₋₆ alkylene)-C(O)—***, —N(H)—(C₁₋₆ alkylene)-C(O)—***, —N(H)—(C₇₋₁₅ alkylene)-C(O)—***, —N(H)—[(C₂₋₄ alkylene)-O—]₂₋₆—(C₁₋₆ alkylene)-***, —N(H)—[(C₂₋₄ alkylene)-O—]₇₋₁₅—(C₁₋₆ alkylene)-***, —N(H)—(C₂₋₉ alkylene)-O—(C₁₋₆ alkylene)-C(O)N(C₁₋₆ alkyl)-(C₁₋₆ alkylene)-***, —N(H)—(C₂₋₉ alkylene)-O—(C₁₋₆ alkylene)-C(O)N(H)—(C₁₋₆ alkylene)-***, —N(H)—[(C₂₋₄ alkylene)-O—]2-6-(C₁₋₆ alkylene)-N(H)—(C₁₋₆ alkylene)-***, —N(H)—[(C₂₋₄ alkylene)-O—]₇₋₁₅(C₁₋₆ alkylene)-N(H)—(C₁₋₆ alkylene)-***, —N(H)—[(C₂₋₄ alkylene)-O—]₂₋₆—(C₁₋₆ alkylene)-N(C₁₋₆ alkyl)-(C₁₋₆ alkylene)-***, or —N(H)—[(C₂₋₄ alkylene)-O—]₇₋₁₅—(C₁₋₆ alkylene)-N(C₁₋₆ alkyl)-(C₁₋₆ alkylene)-***, where *** is a point of attachment to TPL.

84. The compound of any one of claims 1-71, wherein L is —N(H)—(C₂₋₉ alkylene)-O—(C₁₋₆ alkylene)-C(O)—***, —N(H)—(C₁₀₋₂₀ alkylene)-O—(C₁₋₆ alkylene)-C(O)—***, —N(H)—[CH₂CH₂—O—]₂₋₆—(C₁₋₆ alkylene)-C(O)—***, —N(H)—[CH₂CH₂—O—]₇₋₁₅—(C₁₋₆ alkylene)-C(O)—***, —N(H)—(C₁₋₆ alkylene)-C(O)—***, —N(H)—(C₇₋₁₅ alkylene)-C(O)—***, —N(H)—[CH₂CH₂—O—]₂₋₆—(C₁₋₆ alkylene)-***, —N(H)—[CH₂CH₂—O—]₇₋₁₅—(C₁₋₆ alkylene)-***, —N(H)—(C₂₋₉ alkylene)-O—(C₁₋₆ alkylene)-C(O)N(C₁₋₆ alkyl)-(C₁₋₆ alkylene)-***, —N(H)—(C₂₋₉ alkylene)-O—(C₁₋₆ alkylene)-C(O)N(H)—(C₁₋₆ alkylene)-***, —N(H)—[CH₂CH₂—O—]₂₋₆—(C₁₋₆ alkylene)-N(H)—(C₁₋₆ alkylene)-***, —N(H)—[CH₂CH₂—O—]₇₋₁₅—(C₁₋₆ alkylene)-N(H)—(C₁₋₆ alkylene)-***, —N(H)—[CH₂CH₂—O—]₂₋₆—(C₁₋₆ alkylene)-N(C₁₋₆ alkyl)-(C₁₋₆ alkylene)-***, or —N(H)—[CH₂CH₂—O—]₇₋₁₅—(C₁₋₆ alkylene)-N(C₁₋₆ alkyl)-(C₁₋₆ alkylene)-***, where *** is a point of attachment to TPL.

85. The compound of any one of claims 1-71, wherein L is —N(H)—[(C₂₋₄ alkylene)-O—]₂₋₆—(C₁₋₆ alkylene)-C(O)—***, —N(H)—[(C₂₋₄ alkylene)-O—]₇₋₁₅—(C₁₋₆ alkylene)-C(O)—***, —N(H)—(C₁₋₆ alkylene)-N(C₁₋₆ alkyl)C(O)—(C₁₋₆ alkylene)***, —N(H)—(C₁₋₆ alkylene)-N(H)C(O)—(C₁₋₆ alkylene)***, —N(H)—(C₂₋₆ alkylene)-***, —N(H)—(C₇₋₁₅ alkylene)-***, —N(C₁₋₆ alkyl)-(C₂₋₆ alkylene)-***, —N(C₁₋₆ alkyl)-(C₇₋₁₅ alkylene)-***, —N(H)—[(C₂₋₄ alkylene)-O—]₂₋₆—(C₁₋₆ alkylene)-***, —N(H)—[(C₂₋₄ alkylene)-O—]₇₋₁₅—(C₁₋₆ alkylene)-***, —N(H)—(C₁₋₆ alkylene)-(3-6 membered heterocycloalkylene)-(C₁₋₆ alkylene)-N(C₁₋₆ alkyl)-(C₁₋₆ alkylene)-***, —N(H)—(C₁₋₆ alkylene)-(3-6 membered heterocycloalkylene)-(C₁₋₆ alkylene)-N(H)—(C₁₋₆ alkylene)-***, —N(H)—(C₂₋₆ alkylene)-N(H)—(C₁₋₆ alkylene)-***, or —N(H)—(C₂₋₆ alkylene)-N(C₁₋₆ alkyl)-(C₁₋₆ alkylene)-***, where *** is a point of attachment to TPL.

86. The compound of any one of claims 1-71, wherein L is —N(H)—[CH₂CH₂—O—]₂₋₆—(C₁₋₆ alkylene)-C(O)—***, —N(H)—[CH₂CH₂—O—]₇₋₁₅—(C₁₋₆ alkylene)-C(O)—***, —N(H)—(C₁₋₆ alkylene)-N(C₁₋₆ alkyl)C(O)—(C₁₋₆ alkylene)***, —N(H)—(C₁₋₆ alkylene)-N(H)C(O)—(C₁₋₆ alkylene)***, —N(H)—(C₂₋₆ alkylene)-***, —N(H)—(C₇₋₁₅ alkylene)-***, —N(C₁₋₆ alkyl)-(C₂₋₆ alkylene)-***, —N(C₁₋₆ alkyl)-(C₇₋₁₅ alkylene)-***, —N(H)—[CH₂CH₂—O—]₂₋₆—(C₁₋₆ alkylene)-***, —N(H)—[CH₂CH₂—O—]₇₋₁₅—(C₁₋₆ alkylene)-***, —N(H)—(C₁₋₆ alkylene)-(3-6 membered heterocycloalkylene)-(C₁₋₆ alkylene)-N(C₁₋₆ alkyl)-(C₁₋₆ alkylene)-***, —N(H)—(C₁₋₆ alkylene)-(3-6 membered heterocycloalkylene)-(C₁₋₆ alkylene)-N(H)—(C₁₋₆ alkylene)-***, —N(H)—(C₂₋₆ alkylene)-N(H)—(C₁₋₆ alkylene)-***, or —N(H)—(C₂₋₆ alkylene)-N(C₁₋₆ alkyl)-(C₁₋₆ alkylene)-***, where *** is a point of attachment to TPL.

87. The compound of any one of claims 1-71, wherein L is —[(C₂₄ alkylene)-O—]₂₋₆—(C₁₋₆ alkylene)-***, —[(C₂₋₄ alkylene)-O—]₇₋₁₅—(C₁₋₆ alkylene)-***, —[(C₂₋₄ alkylene)-O—]₂₋₆—(C₁₋₆ alkylene)-N(C₁₋₆ alkyl)(C₁₋₆ alkylene)-***, —[(C₂₋₄ alkylene)-O—]₇₋₁₅—(C₁₋₆ alkylene)-N(C₁₋₆ alkyl)(C₁₋₆ alkylene)-***, —[(C₂₋₄ alkylene)-O—]₂₋₆—(C₁₋₆ alkylene)-N(H)(C₁₋₆ alkylene)-***, —[(C₂₋₄ alkylene)-O—]₇₋₁₅—(C₁₋₆ alkylene)-N(H)(C₁₋₆ alkylene)-***, —(C₁₋₉ alkylene)-C(O)N(H)—(C₁₋₆ alkylene)-***, —(C₁₋₉ alkylene)-N(H)C(O)—(C₁₋₆ alkylene)-***, —(C₁₋₉ alkylene)-C(O)N(H)—[(C₂₋₄ alkylene)-O—]₂₋₆—(C₁₋₆ alkylene)-***, —(C₁₋₉ alkylene)-N(H)C(O)—[(C₂₋₄ alkylene)-O—]₂₋₆—(C₁₋₆ alkylene)-***, —(C₁₋₉ alkylene)-C(O)N(H)—[(C₂₋₄ alkylene)-O—]₇₋₁₅—(C₁₋₆ alkylene)-***, —(C₁₋₉ alkylene)-N(H)C(O)—[(C₂₋₄ alkylene)-O—]₇₋₁₅—(C₁₋₆ alkylene)-***, —(C₁₋₉ alkylene)-C(O)N(H)—[(C₂₋₄ alkylene)-O—]₂₋₆—(C₁₋₆ alkylene)-N(C₁₋₆ alkyl)-(C₁₋₆ alkylene)-***, —(C₁₋₉ alkylene)-N(H)C(O)—[(C₂₋₄ alkylene)-O—]₂₋₆—(C₁₋₆ alkylene)-N(C₁₋₆ alkyl)-(C₁₋₆ alkylene)-***, —(C₁₋₉ alkylene)-C(O)N(H)—[(C₂₋₄ alkylene)-O—]₇₋₁₅—(C₁₋₆ alkylene)-N(C₁₋₆ alkyl)-(C₁₋₆ alkylene)-***, or —(C₁₋₉ alkylene)-N(H)C(O)—[(C₂₋₄ alkylene)-O—]₇₋₁₅—(C₁₋₆ alkylene)-N(C₁₋₆ alkyl)-(C₁₋₆ alkylene)-***, where *** is a point of attachment to TPL.

88. The compound of any one of claims 1-71, wherein L is —[CH₂CH₂—O—]₂₋₆—(C₁₋₆ alkylene)-***, —[CH₂CH₂—O—]₇₋₁₅—(C₁₋₆ alkylene)-***, —[CH₂CH₂—O—]₂₋₆—(C₁₋₆ alkylene)-N(C₁₋₆ alkyl)(C₁₋₆ alkylene)-***, —[CH₂CH₂—O—]₇₋₁₅—(C₁₋₆ alkylene)-N(C₁₋₆ alkyl)(C₁₋₆ alkylene)-***, —[CH₂CH₂—O—]₂₋₆—(C₁₋₆ alkylene)-N(H)(C₁₋₆ alkylene)-***, —[CH₂CH₂—O—]₇₋₁₅—(C₁₋₆ alkylene)-N(H)(C₁₋₆ alkylene)-***, —(C₁₋₉ alkylene)-C(O)N(H)—(C₁₋₆ alkylene)-***, —(C₁₋₉ alkylene)-N(H)C(O)—(C₁₋₆ alkylene)-***, —(C₁₋₉ alkylene)-C(O)N(H)—[CH₂CH₂—O—]₂₋₆—(C₁₋₆ alkylene)-***, —(C₁₋₉ alkylene)-N(H)C(O)—[CH₂CH₂—O—]₂₋₆—(C₁₋₆ alkylene)-***, —(C₁₋₉ alkylene)-C(O)N(H)—[CH₂CH₂—O—]₇₋₁₅—(C₁₋₆ alkylene)-***, —(C₁₋₉ alkylene)-N(H)C(O)—[CH₂CH₂—O—]₇₋₁₅—(C₁₋₆ alkylene)-***, —(C₁₋₆ alkylene)-C(O)N(H)—[CH₂CH₂—O—]₂₋₆—(C₁₋₆ alkylene)-N(C₁₋₆ alkyl)-(C₁₋₆ alkylene)-***, —(C₁₋₉ alkylene)-N(H)C(O)—[CH₂CH₂—O—]₂₋₆—(C₁₋₆ alkylene)-N(C₁₋₆ alkyl)-(C₁₋₆ alkylene)-***, —(C₁₋₉ alkylene)-C(O)N(H)—[CH₂CH₂—O—]₇-15—(C₁₋₆ alkylene)-N(C₁₋₆ alkyl)-(C₁₋₆ alkylene)-***, or —(C₁₋₉ alkylene)-N(H)C(O)—[(CH₂CH₂—O—]₇₋₁₅—(C₁₋₆ alkylene)-N(C₁₋₆ alkyl)-(C₁₋₆ alkylene)-***, where *** is a point of attachment to TPL.

89. The compound of any one of claims 1-71, wherein L is —N(H)—[(C₂₋₄ alkylene)-O—]₂₋₆—(C₁₋₆ alkylene)-N(H)—***, —N(H)—[(C₂₋₄ alkylene)-O—]₇₋₁₅—(C₁₋₆ alkylene)-N(H)—***, —N(C₁₋₆ alkyl)-[(C₂₋₄ alkylene)-O—]₂₋₆—(C₁₋₆ alkylene)-N(H)—***, —N(C₁₋₆ alkyl)-[(C₂₋₄ alkylene)-O—]₇₋₁₅—(C₁₋₆ alkylene)-N(H)—***, —N(C₁₋₆ alkyl)-[(C₂₋₄ alkylene)-O—]₂₋₆—(C₁₋₆ alkylene)-N(C₁₋₆ alkyl)-***, or —N(C₁₋₆ alkyl)-[(C₂₋₄ alkylene)-O—]₇₋₁₅—(C₁₋₆ alkylene)-N(C₁₋₆ alkyl)-***, where** * is a point of attachment to TPL.

90. The compound of any one of claims 1-71, wherein L is —N(H)—[CH₂CH₂—O—]₂₋₆—(C₁₋₆ alkylene)-N(H)—***, —N(H)—[CH₂CH₂—O—]₇₋₁₅—(C₁₋₆ alkylene)-N(H)—***, —N(C₁₋₆ alkyl)-[CH₂CH₂—O—]₂₋₆—(C₁₋₆ alkylene)-N(H)—***, —N(C₁₋₆ alkyl)-[CH₂CH₂—O—]₇₋₁₅—(C₁₋₆ alkylene)-N(H)—***, —N(C₁₋₆ alkyl)-[CH₂CH₂—O—]₂₋₆—(C₁₋₆ alkylene)-N(C₁₋₆ alkyl)-***, or —N(C₁₋₆ alkyl)-[CH₂CH₂—O—]₇₋₁₅—(C₁₋₆ alkylene)-N(C₁₋₆ alkyl)-***, where *** is a point of attachment to TPL.

91. The compound of any one of claims 1-71, wherein L is —(C₂₋₁₀ alkylene)-(OCH₂CH₂)₂₋₄—O-(3-6 membered saturated heterocyclylene containing 1 or 2 heteroatoms independently selected from nitrogen and oxygen)-(C₁₋₆ alkylene)-***, —(C₂₋₁₀ alkylene)-(3-6 membered saturated heterocyclylene containing 1 or 2 heteroatoms independently selected from nitrogen and oxygen)-(C₁₋₅ alkylene)-***, —(C₂₋₁₀ alkylene)-N(H)—(C₁₋₅alkylene)-***, —(C₂₋₁₀ alkylene)-N(C₁₋₆ alkyl)-(C₁₋₅ alkylene)-***, —N(H)—(C₁₋₅ alkylene)-***, —(CH₂CH₂O)_{1 -4}—(C₁₋₄ alkylene)-***, —(CH₂CH₂O)_{1 -4}—(C₁₋₄ alkylene)-N(C₁₋₆ alkyl)-(C₁₋₆ alkylene)-***, —N(H)—(C₂₋₆ alkylene)-(3-6 membered saturated heterocyclylene containing 1 or 2 heteroatoms independently selected from nitrogen and oxygen)-(C₁₋₅ alkylene)-***, —N(H)—(C₂₋₆ alkylene)-(OCH₂CH₂)₁₋₄—O-(3-6 membered saturated heterocyclylene containing 1 or 2 heteroatoms independently selected from nitrogen and oxygen)-(C₁₋₅alkylene)-***, —N(H)—[—CH₂CH₂O—]₂₋₆—(C₁₋₆ alkylene)-N(C₁₋₆ alkyl)-(C₁₋₆ alkylene)-***, —(C₁₋₆ alkylene)-(3-6 membered saturated heterocyclylene containing 1 or 2 heteroatoms independently selected from nitrogen and oxygen)-(C₁₋₆ alkylene)-***, —N(H)—(C₂₋₁₀ alkylene)-***, —(C₁₋₆ alkylene)-(3-6 membered saturated heterocyclylene containing 1 or 2 heteroatoms independently selected from nitrogen and oxygen)-(C₁₋₆ alkylene)-N(C₁₋₆ alkyl)-(C₁₋₆ alkylene)-***, —N(H)—[CH₂CH₂—O—]₂₋₆—(C₁₋₆ alkylene)-N(C₁₋₆ alkyl)-(C₁₋₆ alkylene)-***, —N(H)—[CH₂CH₂—O—]₂₋₁₀—(C₁₋₆ alkylene)-***, —N(H)—[CH₂CH₂—O—]₂₋₆—(C₁₋₆ alkylene)-N(C₁₋₆ alkyl)C(O)—(C₁₋₆ alkylene)-***, —[CH₂CH₂—O—]₁₋₆—(C₁₋₆ alkylene)-N(C₁₋₆ alkyl)-***, —[CH₂CH₂—O—]₁₋₆—(C₁₋₆ alkylene)-N(H)—***, or —(C₂₋₁₀ alkylene)-(OCH₂CH₂)₂₋₆—(C₃₋₆ cycloalkylene)-***, where *** is a point of attachment to TPL.

92. A compound in Table 1 or 2, or a pharmaceutically acceptable salt thereof.

93. A compound in Table 1-A or 2-A, or a pharmaceutically acceptable salt thereof.

94. A pharmaceutical composition comprising a compound of any one of claims 1-93 and a pharmaceutically acceptable carrier.

95. A method of treating cancer, comprising administering to a patient in need thereof a therapeutically effective amount of a compound of any one of claims 1-93 to treat the cancer.

96. The method of claim 95, wherein the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, or leukemia.

97. A method of causing death of a cancer cell, comprising contacting a cancer cell with an effective amount of a compound of any one of claims 1-93 to cause death of the cancer cell.

98. The method of claim 97, wherein the cancer cell is selected from an ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, or leukemia cell.
